(19) United States
(12) Patent Application Publication
(10) Pub. No.: US 2023/0391786 A1
(43) Pub. Date: Dec. 7, 2023

(54) cGAS INHIBITORS

(71) Applicant: Eli Lilly and Company, Indianapolis, IN (US)

(72) Inventors: Adel AHMED, Fishers, IN (US); Christina MARTINEZ BROKAW, Zionsville, IN (US); Cheryl Ann CARSON, Indianapolis, IN (US); Scott E. CONNER, Carmel, IN (US); Kevin Charles FORTNER, Indianapolis, IN (US); Jeffry B. FRANCISKOVICH, Zionsville, IN (US); Douglas L. GERNERT, Fishers, IN (US); Steven J. GREEN, Indianapolis, IN (US); Charles W. LUGAR, III, McCordsville, IN (US); Jothirajah MARIMUTHU, Carmel, IN (US); Shanthi NAGARAJAN, San Diego, CA (US); Christodoulos NICOLAOU, Carmel, IN (US); Emmanuel ONOBUN, Indianapolis, IN (US); Stephanie Lange STOUT, Brownsburg, IN (US); Eric G. TROMICZAK, Indianapolis, IN (US); Thibault VARIN, Carmel, IN (US)

(21) Appl. No.: 18/327,647

(22) Filed: Jun. 1, 2023

Related U.S. Application Data
(60) Provisional application No. 63/348,147, filed on Jun. 2, 2022.

(51) Int. Cl. C07D 487/04; C07D 403/04; C07D 417/14; C07D 403/14; C07D 471/04; C07D 413/14

(52) U.S. Cl. CPC C07D 487/04; C07D 403/04; C07D 417/14; C07D 403/14; C07D 471/04; C07D 413/14

(57) ABSTRACT

The present invention relates to novel cGAS inhibitor compounds, to pharmaceutical compositions comprising the compounds, and to methods of using the compounds and compositions to treat certain pathological conditions.

The present invention relates to novel cGAS inhibitor compounds, to pharmaceutical compositions comprising the compounds, and to methods of using the compounds and compositions to treat certain pathological conditions.

Cyclic GAMP-AMP synthase (cGAS) is a critical cytosolic DNA sensor that catalyzes the formation of 2′3′-Cyclic GMP-AMP (cGAMP), a second messenger that binds to Stimulator of interferon genes (STING) to trigger downstream signaling resulting in the production of proinflammatory cytokines and type I interferons (Ablasser, A. et al., Nature, 2013, 498, 380-384, Sun, L., et al., Science, 2013, 339, 786-791). cGAS recognizes dsDNA and retroviral DNA intermediates (Gao, D., et al., Science, 2013, 341, 903-906) in a nonsequence-specific manner and dimerizes to activate its nucleotidyl transferase function (Civril, F., et al., Nature, 2013, 498, 332-337), triggering potent antiviral effector functions through the interferon signature genes induced by the type I interferons. Failure to degrade cytosolic DNA and mitochondrial DNA leakage from cellular stress have also been implicated in pathogenic cGAS activation in diseases such as Aicardi-Goutières syndrome (Gray, E. E., et al., J Immunol, 2015, 195, 1939-1943) and systemic lupus erythematosus (SLE) (Caielli, S., et al., Cell, 2021, 184, 4464-4479). cGAS deficiency rescues the autoimmune phenotype and fatality of TREX1 knockout mice that accumulate DNA in the cytosol, mimicking features of Aicardi-Goutières syndrome (Xiao, N., et al., J Autoimmun, 2019, 100, 84-94). SLE is a heterogenous disease characterize by the widespread loss of tolerance of nuclear antigens such as anti-dsDNA antibodies and a high interferon gene signature. Reduction of the interferon gene signature in clinical trials has been correlated to amelioration of symptoms and interferon signature gene panels has been used both as a biomarker and pharmacodynamic marker in SLE clinical trials (Furie, R, et al., J Clin Invest, 2019, 129, 1359-1371, Smith, M. A., et al., Sci Rep, 2020, 10, 4462, Tanaka, T., et al., Mod Rheumatol, 2022, 00, 1-11). UV light exposure in skin has been shown to activate cGAS (Skopelja-Gardner, S., et al., Sci Rep, 2020, 10, 7908) and elevations in cGAMP has been reported to be elevated in SLE (An, J., et al., Arthritis Rheumatol, 2017, 69, 800-807). In addition, mitochondrial dysfunction in SLE also promotes cGAS activation (Caielli, S., et al., Cell, 2021, 184, 4464-4479). WO 2019/153002 A1 discloses 2,3,4,5-tetrahydro-1H-pyrido[4,3-b]indole inhibitors of cGAS for treating autoinflammatory diseases. However, there is no currently approved cGAS inhibitor available. Nor is there any cGAS inhibitor in the clinic at this time. Various preclinical scaffolds face barriers such as poor cell activity, unsatisfactory potency, and/or unfavorable pharmacokinetic characteristics. There thus remains a need for novel, oral, selective, and/or potent cGAS inhibitors for the treatment of immune-mediated diseases, including cGAS-mediated immune disorders, such as cGAS-mediated aspects of SLE. The present invention provides novel cGAS inhibitors, and compositions thereof, for use in the treatment of such diseases.

The present invention provides novel cGAS inhibitors, and compositions thereof, for use in the treatment of immune-mediated diseases.

Accordingly, the present invention provides a compound of Formula I:

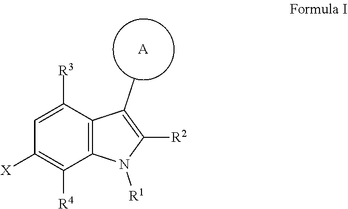

wherein
ring A is 5-membered heteroaryl containing 1-3 nitrogen atoms;
R¹ is H, C₁₋₃ alkyl, —(CH₂)ₙC(O)OH, —(CH₂)ₙNHS(O)₂CH₃ or a 5-membered heteroaryl containing 1-3 nitrogen atoms optionally substituted once with R^{m};
R² is H, C₁₋₃ alkyl, —CH₂NHRⁱ, —CH₂C(O)NHCH₂Rⁱ, —(CH₂)ₙC(O)OH or a 5- to 6-membered heteroaryl containing 1-3 heteroatoms selected from nitrogen and oxygen and optionally substituted with —NH₂;
or R¹ and R², together with the atoms to which they are attached, form a 5- to 7-membered heterocyclic ring containing 1-2 nitrogen atoms, optionally substituted with oxo and optionally substituted with R^{m};
R³ is H, —NH(CH₂)ₙOH, —NHC(O)(CH₂)ₙOH, —NH(CH₂)ₙCN, —NHC(O)CH₃, —NH(CH₂)ₙC(O)NH₂, —O(CH₂)ₙCN, —O(CH₂)ₙC(O)NH₂, —O(CH₂)ₙOH, —O(CH₂)ₙCH₃, —OCH(CH₃)CN, or —NH(C₃₋₆ cycloalkyl) wherein the C₃₋₆ cycloalkyl is optionally substituted with —OH;
X is halo;
R⁴ is halo or —CN;
Rⁱ is acetyl or a 5- to 6-membered heteroaryl containing 1-3 heteroatoms independently selected from nitrogen, oxygen and sulfur and optionally substituted with C₁₋₃ alkyl;
R^{m} is C₁₋₃ alkyl, —CH₂R^{q}, —(CH₂)ₙNH₂, —C(O)NHCH₃, —NHC(O)CH₃, —NHS(O)₂CH₃, —(CH₂)ₙOH, —CH₂C(O)OH, —C(O)CH₃, —C(O)OH, —C(O)(CH₂)ₙOH, —C(O)(CH₂)ₙOCH₃, —C(O)(CH₂)ₙC(O)N(CH₃)₂, —C(O)R^{q}, —C(O)CH₂R^{q}, —OH, —S(O)₂NH₂, —S(O)₂CH₃, tetrazole or pyrrolidin-2-one;
R^{q} is

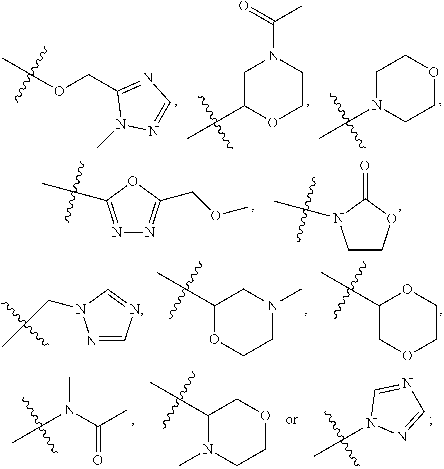

and
n is 1, 2 or 3. In an embodiment of the invention, n is 1 or 2. In a further embodiment, n is 1.

In an embodiment of the invention, ring A is a 5-membered heteroaryl containing 2-3 nitrogen atoms. In a further embodiment, ring A is imidazole, pyrazole or triazole. In another embodiment, ring A is

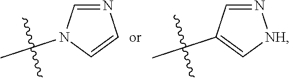 [SMILES: *n1ccnc1 ; *c1cn[nH]c1]

In a further embodiment, ring A is

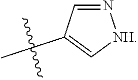 [SMILES: *c1cn[nH]c1]

In an embodiment of the invention, R¹ is H, —CH₃, —CH₂C(O)OH, —CH₂CH₂C(O)OH or a 5-membered heteroaryl containing 2-3 nitrogen atoms and optionally substituted with R^{m}. In a further embodiment, R¹ is

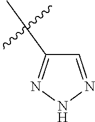 [SMILES: *c1cn[nH]n1]

or

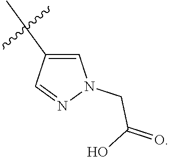 [SMILES: *c1cnn(CC(=O)O)c1]

In a further embodiment R¹ is H.

In an embodiment of the invention, R² is H, —CH₃, —CH₂NHRⁱ, CH₂C(O)NHCH₂Rⁱ, —CH₂CH₂C(O)OH or oxadiazole optionally substituted with —NH₂. In a further embodiment, R² is H.

In an embodiment, Rⁱ is acetyl,

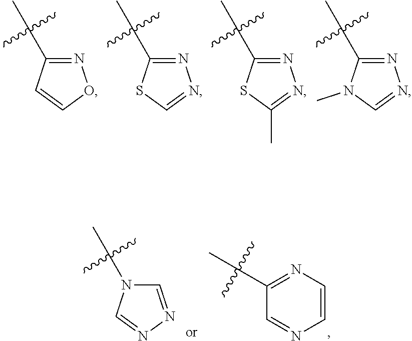

In an embodiment of the invention, R³ is H, —NHCH₂CH₂OH, —OCH₂CN, —NHCH₂C(O)NH₂, —NHCH₂CN, —NHC(O)CH₃, —OCH₂CH₂OH, —OCH₂C(O)NH₂, —OCH₂CH₃, —OCH(CH₃)CN or

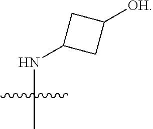 [SMILES: *NC1CC(O)C1]

In an embodiment X is Cl or Br. In a further embodiment X is Cl.

In an embodiment of the invention, R⁴ is F, Cl, Br or —CN. In a further embodiment R⁴ is Cl or —CN. In a further embodiment R⁴ is Cl.

In an embodiment of the invention, when R¹ and R², together with the atoms to which they are attached, form a 5- to 7-membered heterocyclic ring containing 1-2 nitrogen atoms, the 5- to 7-membered heterocyclic ring is

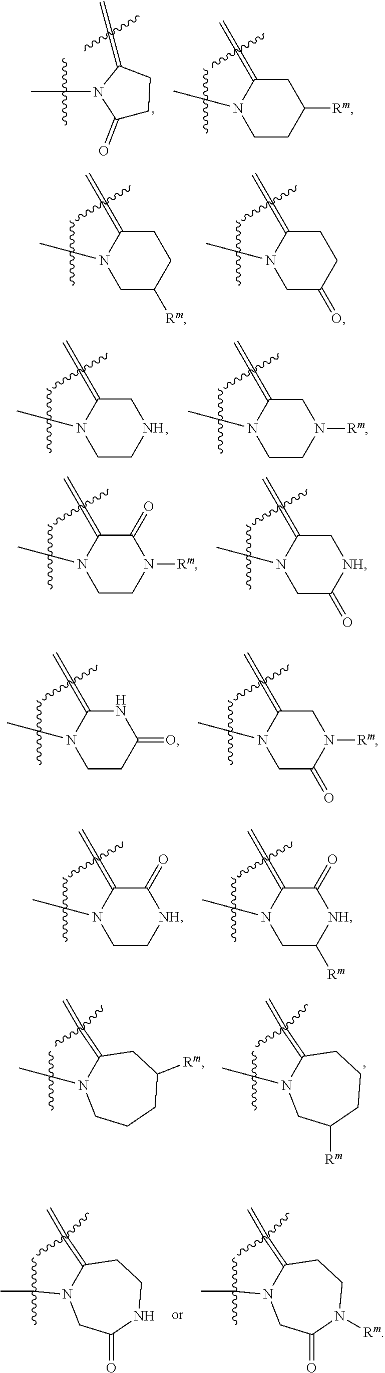

In a further embodiment, when R¹ and R², together with the atoms to which they are attached, form a 5- to 7-membered heterocyclic ring containing 1-2 nitrogen atoms, the 5- to 7-membered heterocyclic ring is

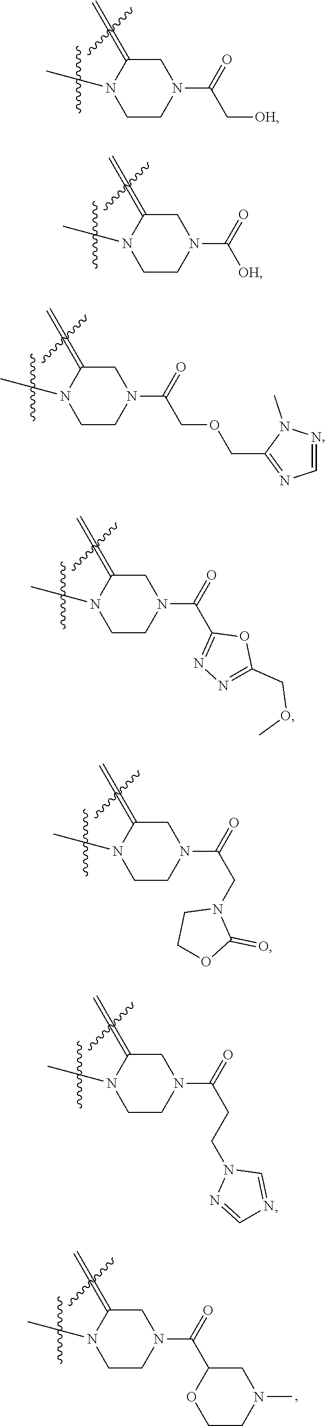

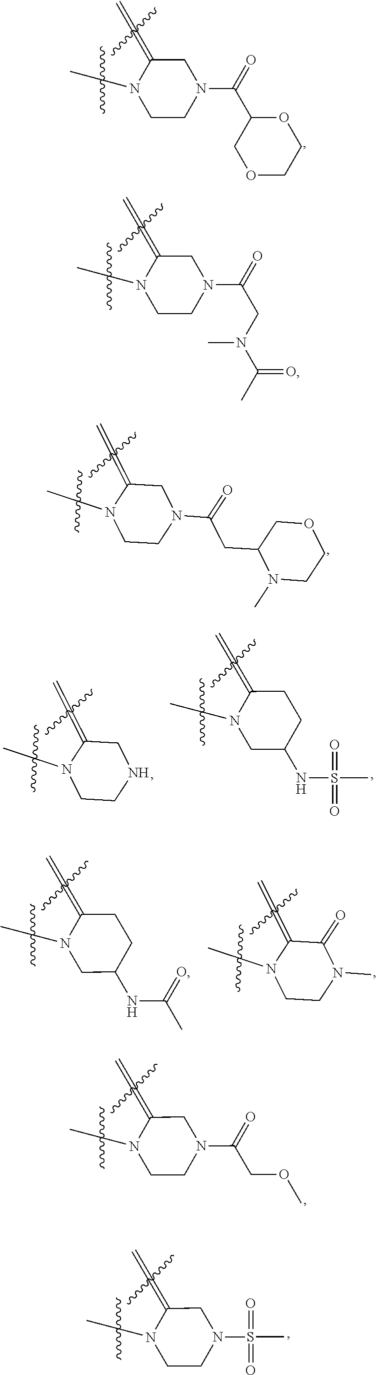

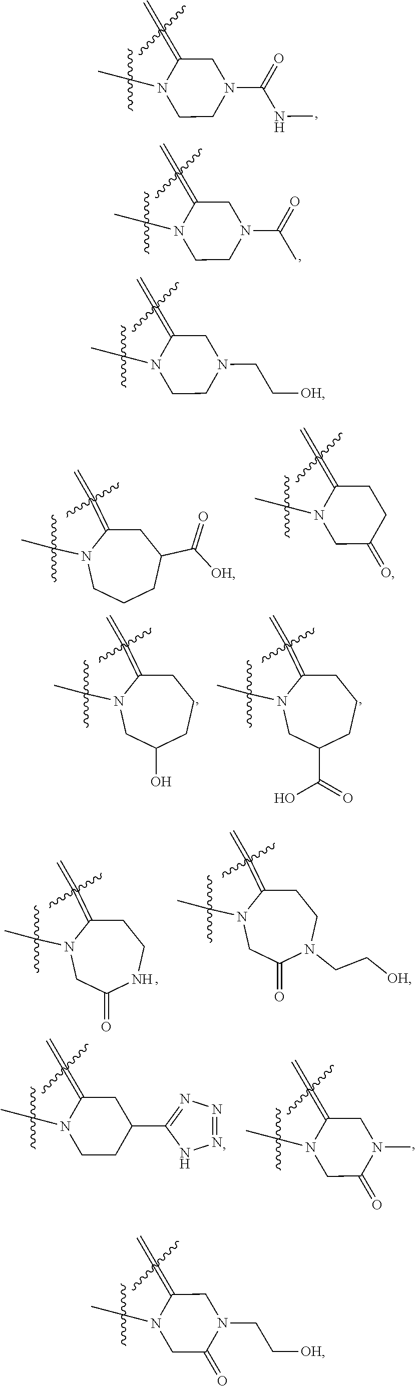

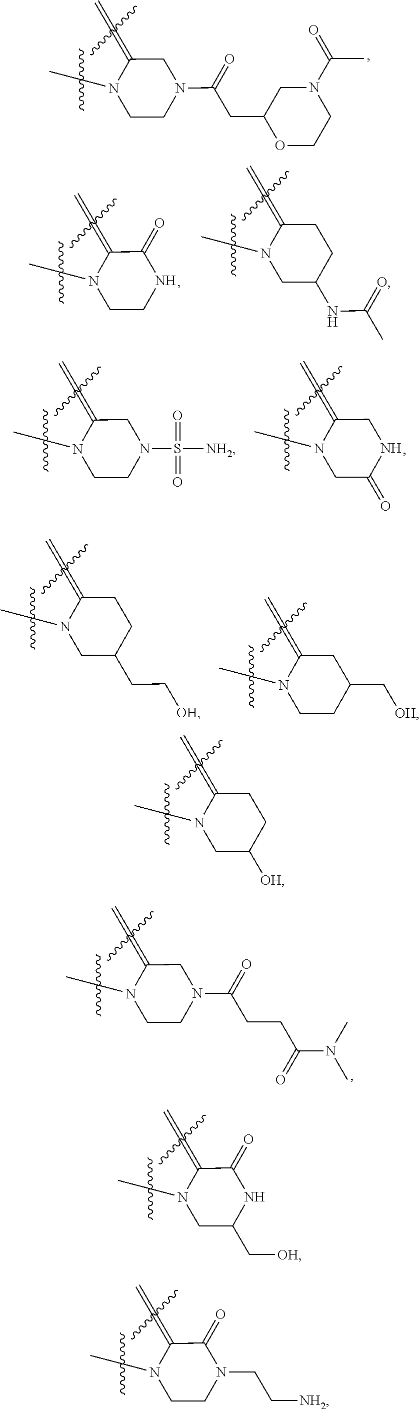

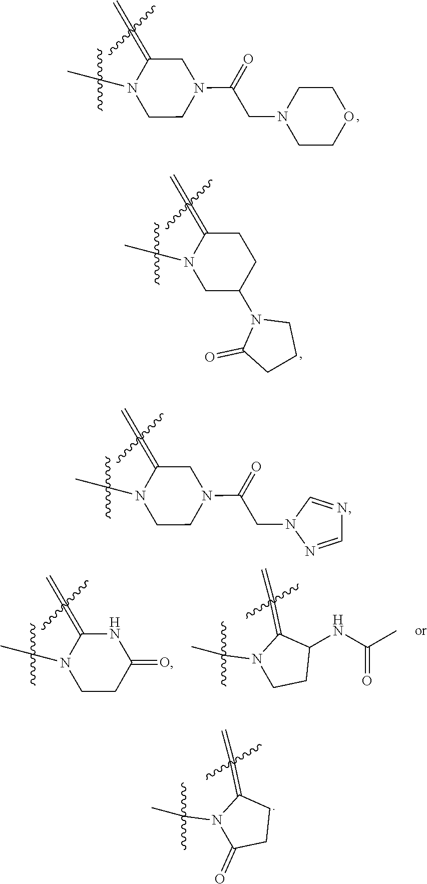 [SMILES: *N1CCN(C(=O)CN2CCOCC2)CC1=* ; *N1CC(N2CCCC2=O)CCC1=* ; *N1CCN(C(=O)Cn2cncn2)CC1=* ; *N1CCC(=O)NC1=* ; *N1CCC(NC(C)=O)C1=* ; *N1C(=*)CCC1=O]

In a further embodiment, when R¹ and R², together with the atoms to which they are attached, form a 5- to 7-membered heterocyclic ring containing 1-2 nitrogen atoms, the 5- to 7-membered heterocyclic ring is

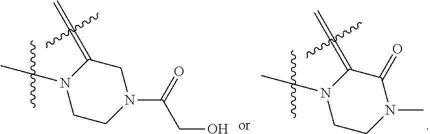

The present invention also provides a compound of Formula II:

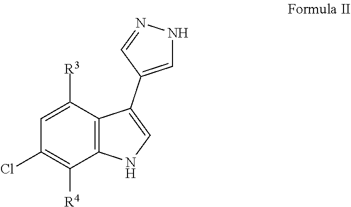

wherein
R³ is H, —NH(CH₂)ₙOH, —NHC(O)(CH₂)ₙOH, —NH(CH₂)ₙCN, —NHC(O)CH₃, —NH(CH₂)ₙC(O)NH₂, —NH(C₃₋₆ cycloalkyl) wherein the C₃₋₆ cycloalkyl is optionally substituted with —OH, —O(CH₂)ₙCN, —O(CH₂)ₙC(O)NH₂, —O(CH₂)ₙOH, —O(CH₂)ₙCH₃, or —OCH(CH₃)CN; and
R⁴ is halo;
or a pharmaceutically acceptable salt thereof.

The present invention also provides a compound of Formula III:

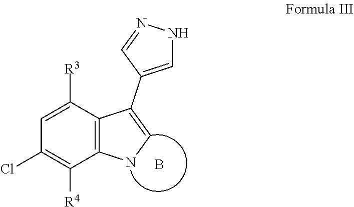

wherein
Ring B is

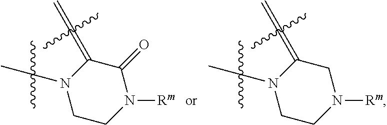

R^{m} is C₁₋₃ alkyl, —CH₂R^{q}, —(CH₂)ₙNH₂, —C(O)NHCH₃, —NHC(O)CH₃, —NHS(O)₂CH₃, —(CH₂)ₙOH, —CH₂C(O)OH, —C(O)CH₃, —C(O)OH, —C(O)(CH₂)ₙOH, —C(O)(CH₂)ₙOCH₃, —C(O)(CH₂)ₙC(O)N(CH₃)₂, —C(O)R^{q}, —C(O)CH₂R^{q}, —OH, —S(O)₂NH₂, —S(O)₂CH₃, tetrazole or pyrrolidin-2-one;
R³ is H, —NH(CH₂)ₙOH, —NHC(O)(CH₂)ₙOH, —NH(CH₂)ₙCN, —NHC(O)CH₃, —NH(CH₂)ₙC(O)NH₂, —NH(C₃₋₆ cycloalkyl) wherein the C₃₋₆ cycloalkyl is optionally substituted with —OH, —O(CH₂)ₙCN, —O(CH₂)ₙC(O)NH₂, —O(CH₂)ₙOH, —O(CH₂)ₙCH₃, or —OCH(CH₃)CN; and
R⁴ is halo;
or a pharmaceutically acceptable salt thereof.

In an embodiment of the invention, the compound is selected from:

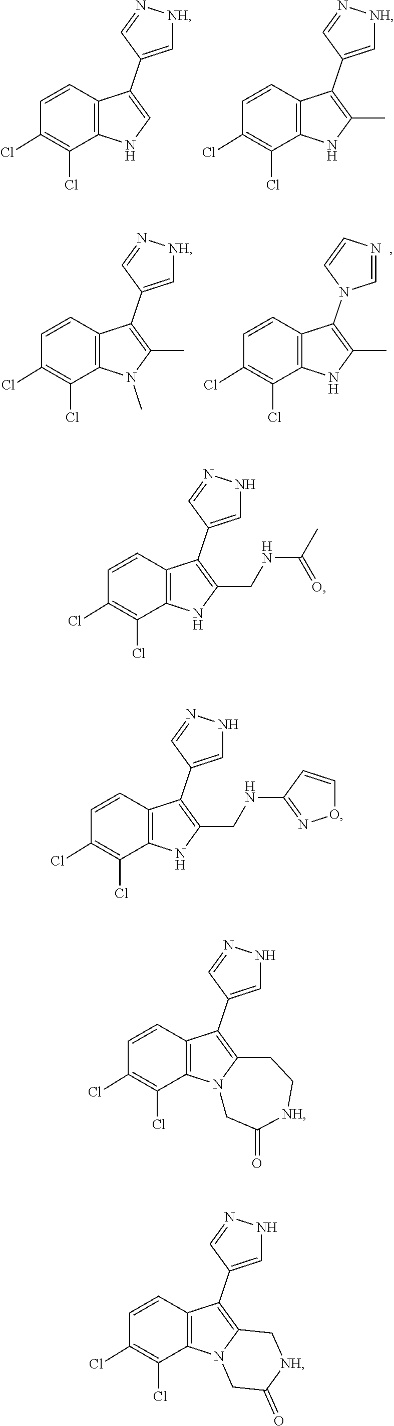

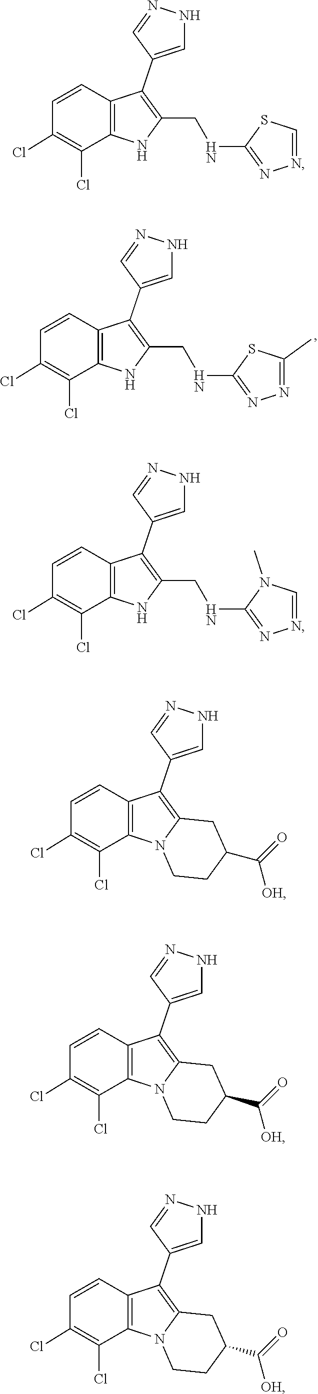 [SMILES: Clc1ccc2c(-c3cn[nH]c3)c(CNc3nncs3)[nH]c2c1Cl ; Cc1nnc(NCc2[nH]c3c(Cl)c(Cl)ccc3c2-c2cn[nH]c2)s1 ; Cn1cnnc1NCc1[nH]c2c(Cl)c(Cl)ccc2c1-c1cn[nH]c1 ; O=C(O)C1CCn2c(c(-c3cn[nH]c3)c3ccc(Cl)c(Cl)c32)C1 ; O=C(O)[C@H]1CCn2c(c(-c3cn[nH]c3)c3ccc(Cl)c(Cl)c32)C1 ; O=C(O)[C@@H]1CCn2c(c(-c3cn[nH]c3)c3ccc(Cl)c(Cl)c32)C1]

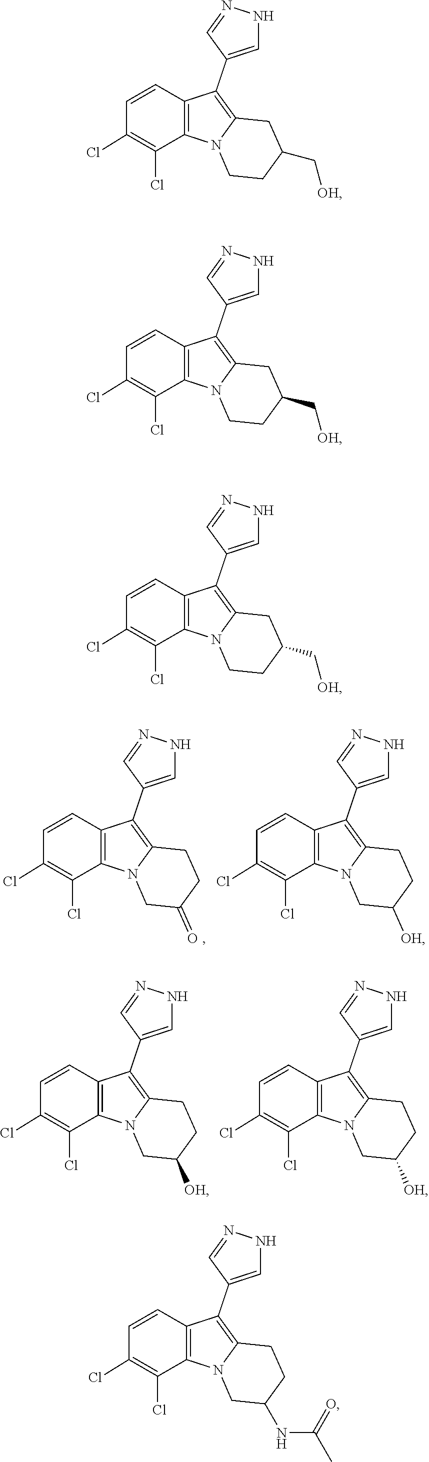

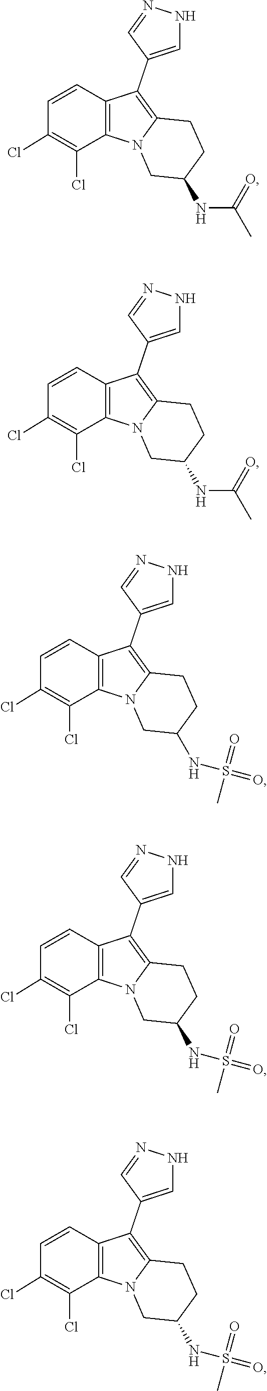 [SMILES: CC(=O)N[C@@H]1CCc2c(-c3cn[nH]c3)c3ccc(Cl)c(Cl)c3n2C1 ; CC(=O)N[C@H]1CCc2c(-c3cn[nH]c3)c3ccc(Cl)c(Cl)c3n2C1 ; CS(=O)(=O)NC1CCc2c(-c3cn[nH]c3)c3ccc(Cl)c(Cl)c3n2C1 ; CS(=O)(=O)N[C@@H]1CCc2c(-c3cn[nH]c3)c3ccc(Cl)c(Cl)c3n2C1 ; CS(=O)(=O)N[C@H]1CCc2c(-c3cn[nH]c3)c3ccc(Cl)c(Cl)c3n2C1]

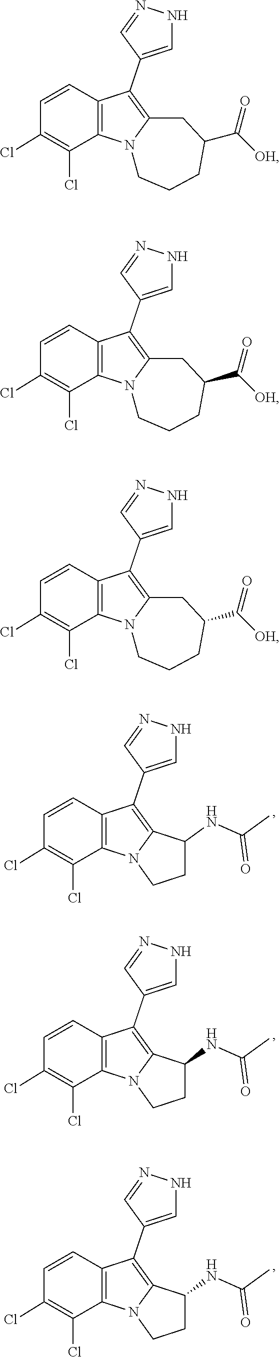 [SMILES: O=C(O)C1CCCn2c(c(-c3cn[nH]c3)c3ccc(Cl)c(Cl)c32)C1 ; O=C(O)[C@H]1CCCn2c(c(-c3cn[nH]c3)c3ccc(Cl)c(Cl)c32)C1 ; O=C(O)[C@@H]1CCCn2c(c(-c3cn[nH]c3)c3ccc(Cl)c(Cl)c32)C1 ; CC(=O)NC1CCn2c1c(-c1cn[nH]c1)c1ccc(Cl)c(Cl)c12 ; CC(=O)N[C@H]1CCn2c1c(-c1cn[nH]c1)c1ccc(Cl)c(Cl)c12 ; CC(=O)N[C@@H]1CCn2c1c(-c1cn[nH]c1)c1ccc(Cl)c(Cl)c12]

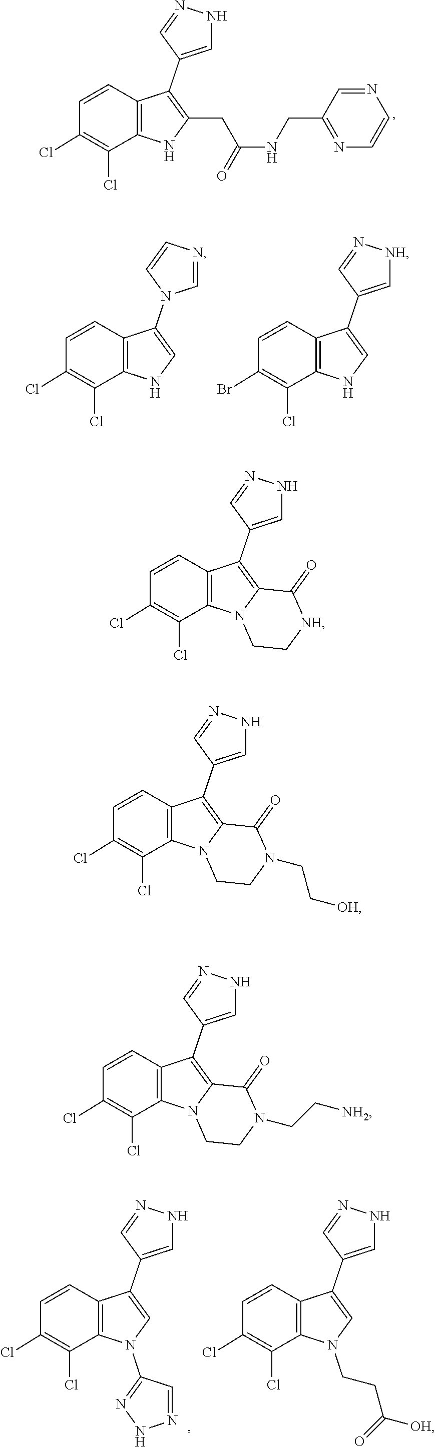

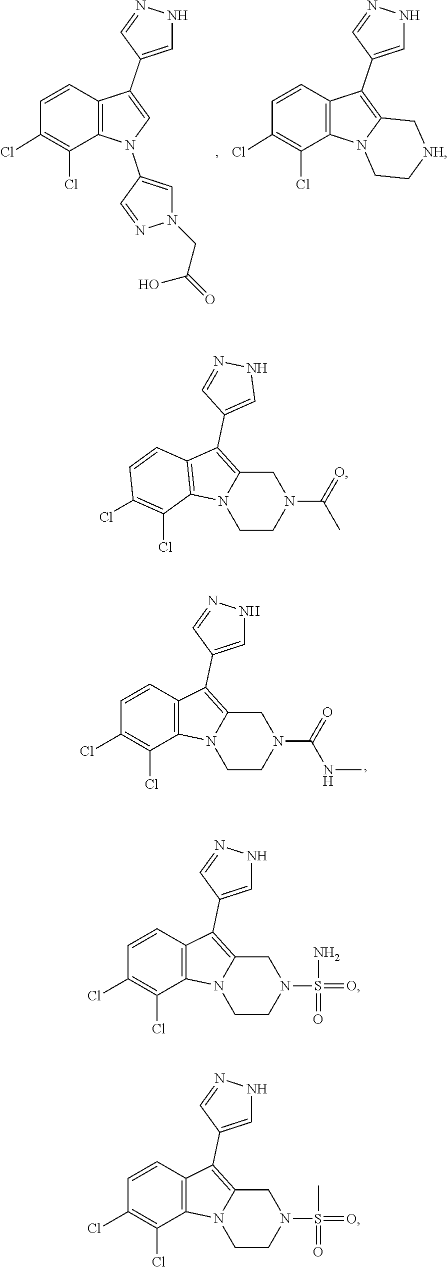 [SMILES: O=C(O)Cn1cc(-n2cc(-c3cn[nH]c3)c3ccc(Cl)c(Cl)c32)cn1 ; Clc1ccc2c(-c3cn[nH]c3)c3n(c2c1Cl)CCNC3 ; CC(=O)N1CCn2c(c(-c3cn[nH]c3)c3ccc(Cl)c(Cl)c32)C1 ; CNC(=O)N1CCn2c(c(-c3cn[nH]c3)c3ccc(Cl)c(Cl)c32)C1 ; NS(=O)(=O)N1CCn2c(c(-c3cn[nH]c3)c3ccc(Cl)c(Cl)c32)C1 ; CS(=O)(=O)N1CCn2c(c(-c3cn[nH]c3)c3ccc(Cl)c(Cl)c32)C1]

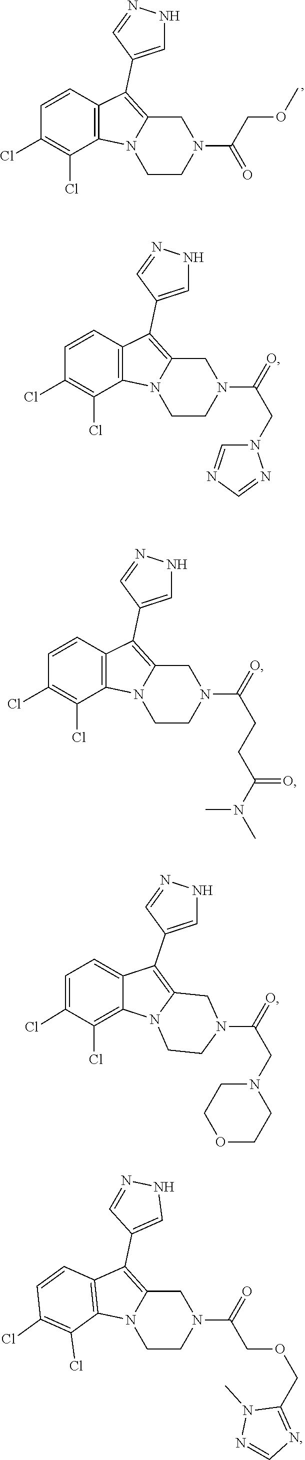 [SMILES: COCC(=O)N1CCn2c(c(-c3cn[nH]c3)c3ccc(Cl)c(Cl)c32)C1 ; O=C(Cn1cncn1)N1CCn2c(c(-c3cn[nH]c3)c3ccc(Cl)c(Cl)c32)C1 ; CN(C)C(=O)CCC(=O)N1CCn2c(c(-c3cn[nH]c3)c3ccc(Cl)c(Cl)c32)C1 ; O=C(CN1CCOCC1)N1CCn2c(c(-c3cn[nH]c3)c3ccc(Cl)c(Cl)c32)C1 ; Cn1ncnc1COCC(=O)N1CCn2c(c(-c3cn[nH]c3)c3ccc(Cl)c(Cl)c32)C1]

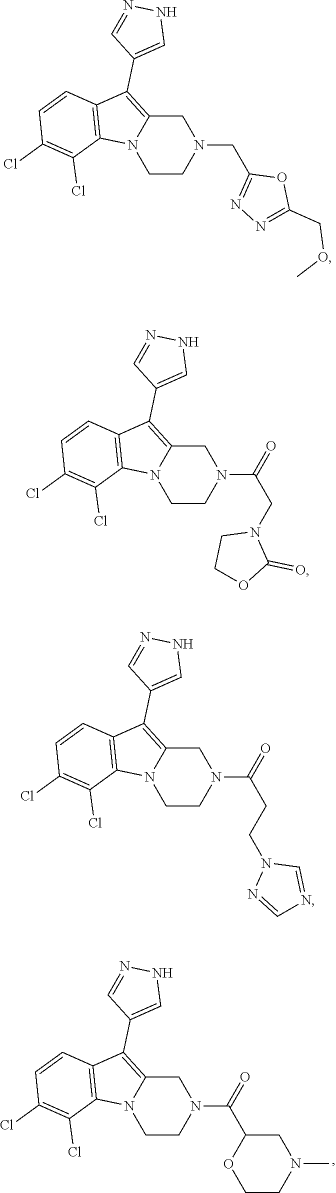 [SMILES: COCc1nnc(CN2CCn3c(c(-c4cn[nH]c4)c4ccc(Cl)c(Cl)c43)C2)o1 ; O=C(CN1CCOC1=O)N1CCn2c(c(-c3cn[nH]c3)c3ccc(Cl)c(Cl)c32)C1 ; O=C(CCn1cncn1)N1CCn2c(c(-c3cn[nH]c3)c3ccc(Cl)c(Cl)c32)C1 ; CN1CCOC(C(=O)N2CCn3c(c(-c4cn[nH]c4)c4ccc(Cl)c(Cl)c43)C2)C1]

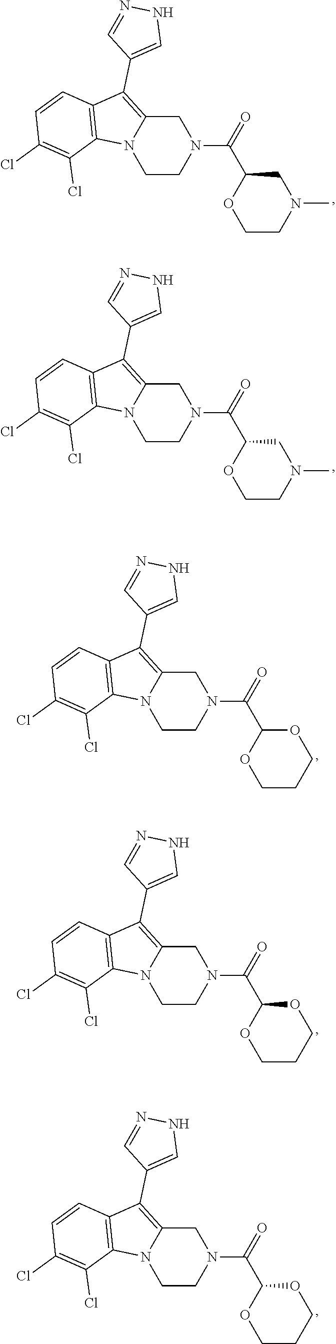

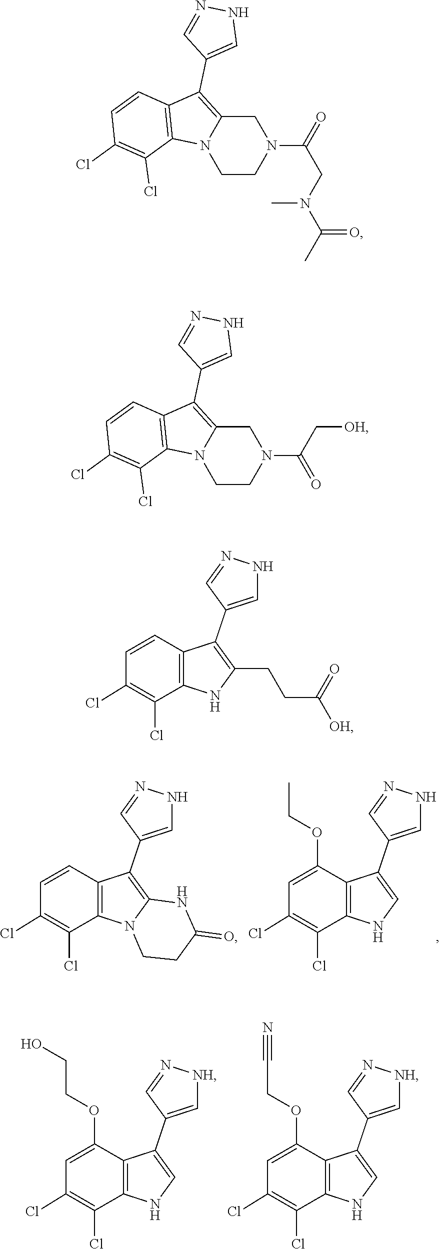

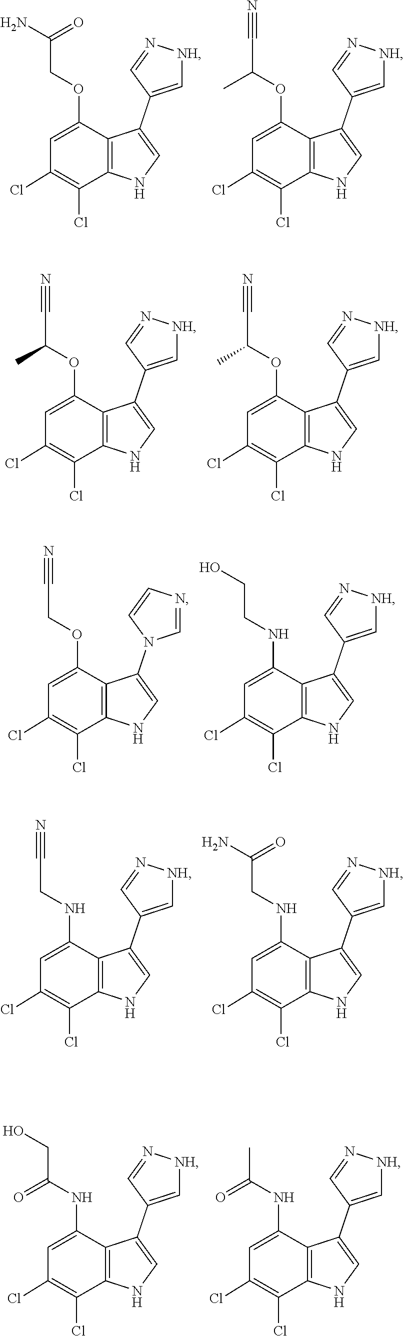

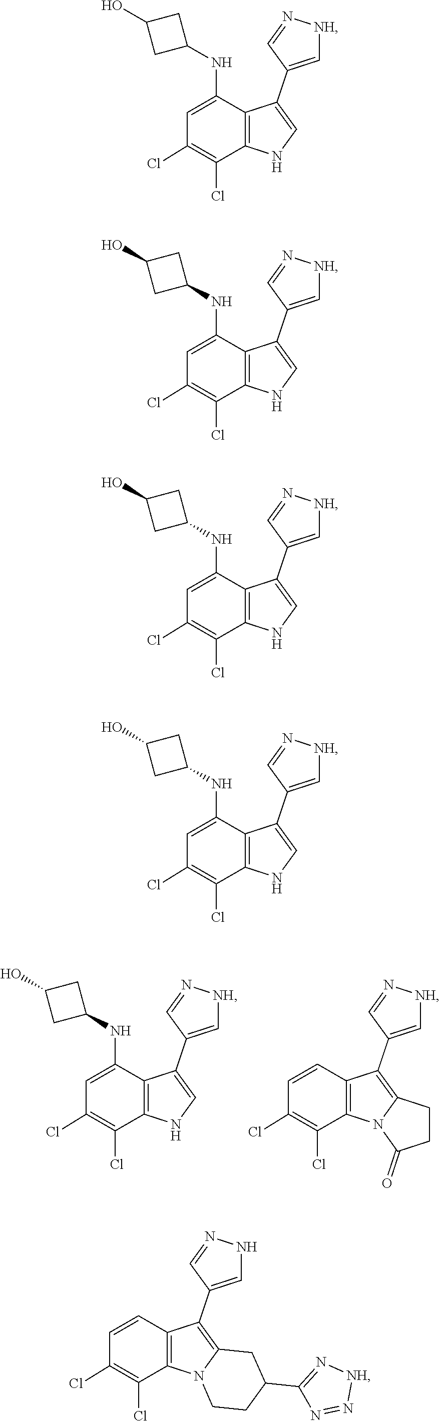

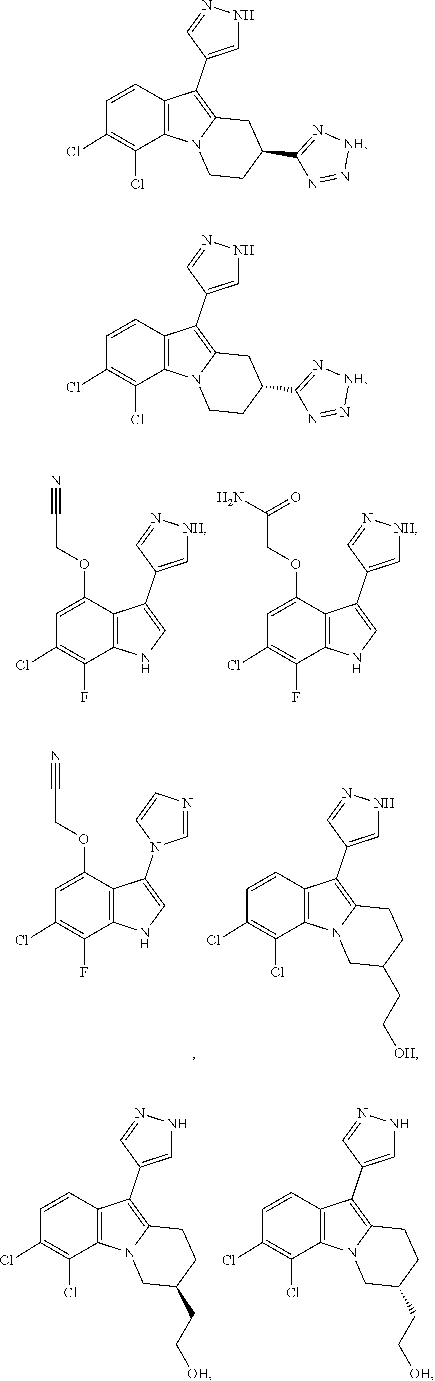

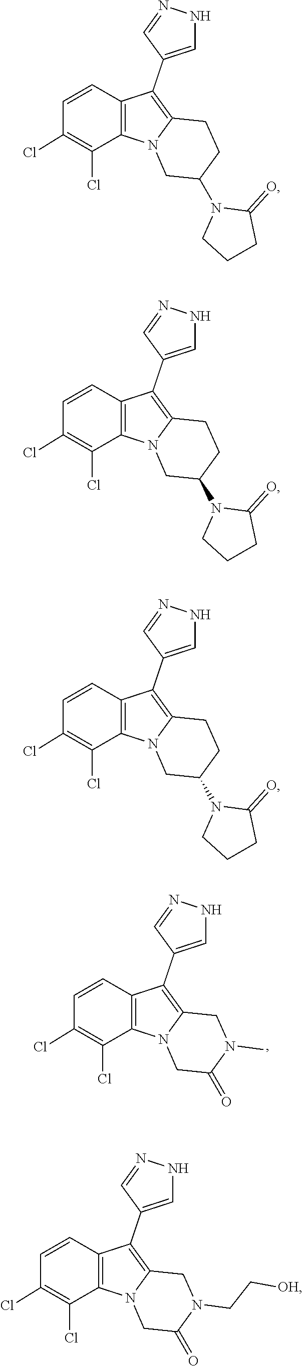 [SMILES: O=C1CCCN1C1CCc2c(-c3cn[nH]c3)c3ccc(Cl)c(Cl)c3n2C1 ; O=C1CCCN1[C@@H]1CCc2c(-c3cn[nH]c3)c3ccc(Cl)c(Cl)c3n2C1 ; O=C1CCCN1[C@H]1CCc2c(-c3cn[nH]c3)c3ccc(Cl)c(Cl)c3n2C1 ; CN1Cc2c(-c3cn[nH]c3)c3ccc(Cl)c(Cl)c3n2CC1=O ; O=C1Cn2c(c(-c3cn[nH]c3)c3ccc(Cl)c(Cl)c32)CN1CCO]

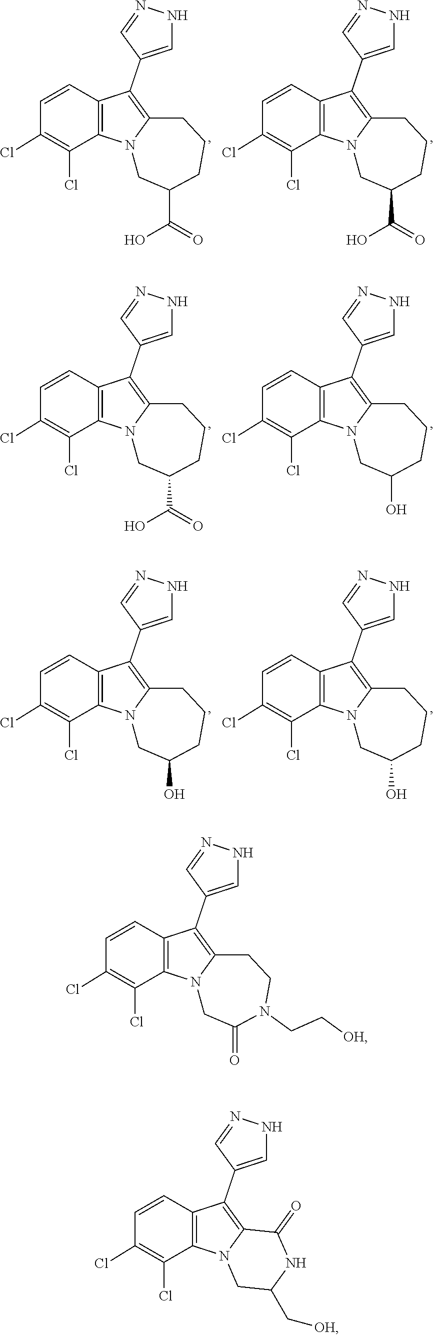

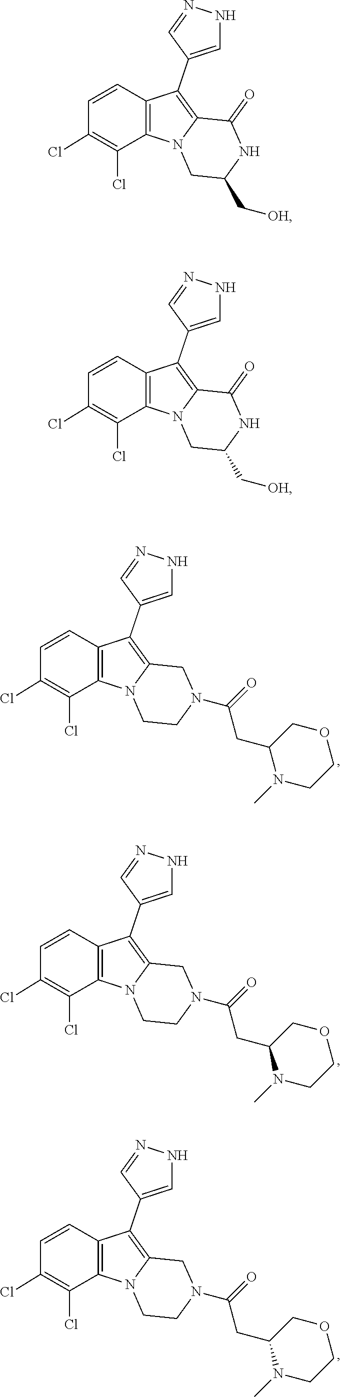 [SMILES: O=C1N[C@@H](CO)Cn2c1c(-c1cn[nH]c1)c1ccc(Cl)c(Cl)c12 ; O=C1N[C@H](CO)Cn2c1c(-c1cn[nH]c1)c1ccc(Cl)c(Cl)c12 ; CN1CCOCC1CC(=O)N1CCn2c(c(-c3cn[nH]c3)c3ccc(Cl)c(Cl)c32)C1 ; CN1CCOC[C@@H]1CC(=O)N1CCn2c(c(-c3cn[nH]c3)c3ccc(Cl)c(Cl)c32)C1 ; CN1CCOC[C@H]1CC(=O)N1CCn2c(c(-c3cn[nH]c3)c3ccc(Cl)c(Cl)c32)C1]

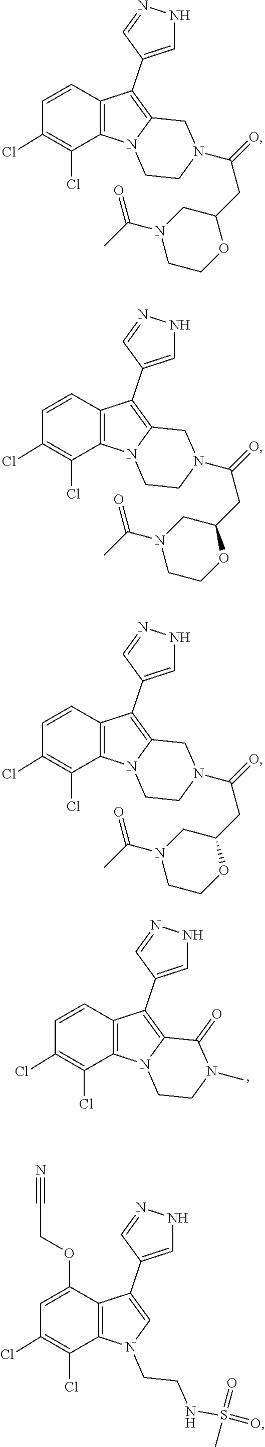

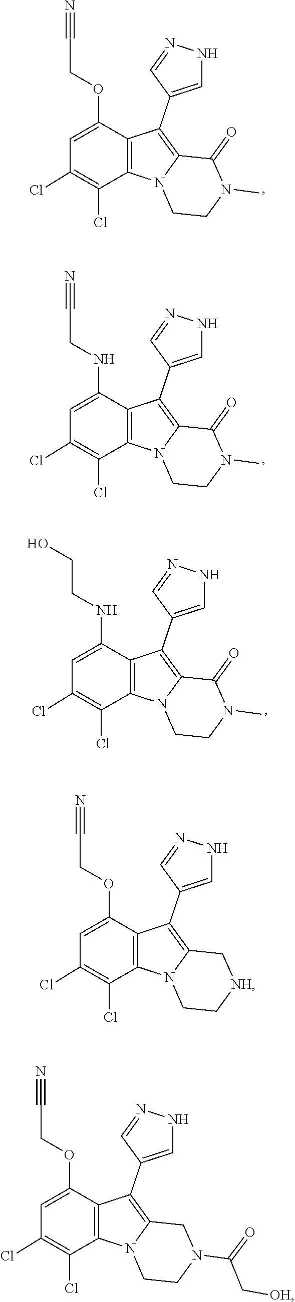 [SMILES: CN1CCn2c(c(-c3cn[nH]c3)c3c(OCC#N)cc(Cl)c(Cl)c32)C1=O ; CN1CCn2c(c(-c3cn[nH]c3)c3c(NCC#N)cc(Cl)c(Cl)c32)C1=O ; CN1CCn2c(c(-c3cn[nH]c3)c3c(NCCO)cc(Cl)c(Cl)c32)C1=O ; N#CCOc1cc(Cl)c(Cl)c2c1c(-c1cn[nH]c1)c1n2CCNC1 ; N#CCOc1cc(Cl)c(Cl)c2c1c(-c1cn[nH]c1)c1n2CCN(C(=O)CO)C1]

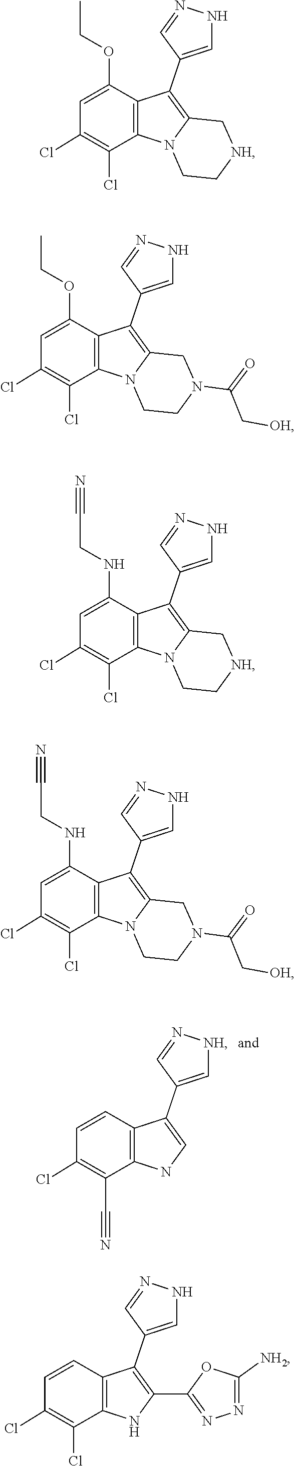

or a pharmaceutically acceptable salt thereof.

The present invention provides a pharmaceutical composition comprising a compound, or a pharmaceutically acceptable salt thereof, according to any of the above embodiments, with one or more pharmaceutically acceptable carriers, diluents, or excipients.

The present invention provides a method of treating an immune-mediated disease in a patient comprising administering to a patient in need of such treatment an effective amount of a compound or salt, or pharmaceutical composition thereof, according to any of the above embodiments.

The present invention also provides a method of treating systemic lupus erythematosus in a patient, comprising administering to a patient in need of such treatment an effective amount of a compound or salt, or pharmaceutical composition according to any of the above embodiments.

Furthermore, the present invention provides a method of treating lupus nephritis in a patient, comprising administering to a patient in need of such treatment an effective amount of a compound or salt, or pharmaceutically composition according to any of the above embodiments.

Additionally, the present invention provides a method of treating dermatomyositis in a patient, comprising administering to a patient in need of such treatment an effective amount of a compound or salt, or pharmaceutically composition according to any of the above embodiments.

The present invention also provides a method of treating Aicardi-Goutières syndrome in a patient, comprising administering to a patient in need of such treatment an effective amount of a compound or salt, or pharmaceutically composition according to any of the above embodiments.

Furthermore, the present invention provides a method of treating a disease associated with cGAS activation in a patient, comprising administering to a patient in need of such treatment an effective amount of a compound or salt, or pharmaceutically composition according to any of the above embodiments.

The present invention provides a compound, or a pharmaceutically acceptable salt thereof, according to any one of the above embodiments for use in therapy.

Furthermore, the present invention provides a compound, or a pharmaceutically acceptable salt thereof, according to any one of the above embodiments for use in the treatment of an immune-mediated disease.

In addition, the present invention provides a compound, or a pharmaceutically acceptable salt thereof, according to any one of the above embodiments for use in the treatment of systemic lupus erythematosus.

The present invention also provides a compound, or a pharmaceutically acceptable salt thereof, according to any one of the above embodiments for use in the treatment of lupus nephritis.

Furthermore, the present invention provides a compound, or a pharmaceutically acceptable salt thereof, according to any one of the above embodiments for use in the treatment of dermatomyositis.

Additionally, the present invention provides a compound, or a pharmaceutically acceptable salt thereof, according to any one of the above embodiments for use in the treatment of Aicardi-Goutières syndrome.

The present invention provides a compound, or pharmaceutically acceptable salt thereof, according to any one of the above embodiments, for the manufacture of a medicament for the treatment of an immune-mediated disease.

In addition, the present invention provides a compound, or pharmaceutically acceptable salt thereof, according to any one of the above embodiments, for the manufacture of a medicament for the treatment of systemic lupus erythematosus.

Furthermore, the present invention provides a compound, or pharmaceutically acceptable salt thereof, according to any one of the above embodiments, for the manufacture of a medicament for the treatment of lupus nephritis.

The present invention also provides a compound, or pharmaceutically acceptable salt thereof, according to any one of the above embodiments, for the manufacture of a medicament for the treatment of dermatomyositis.

Additionally, the present invention provides a compound, or pharmaceutically acceptable salt thereof, according to any one of the above embodiments, for the manufacture of a medicament for the treatment of Aicardi-Goutières syndrome.

Further embodiments as described below. When a later embodiment refers to a previous “embodiment X”, such reference also includes references to “embodiment X-1”, “embodiment X-2”, “embodiment X-3”, and so on, unless such later embodiment cannot be properly construed as a dependent embodiment (e.g. falling outside the scope of the referenced embodiment or having improper antecedent basis). For example, when “Embodiment 8” below refers to “embodiment 1, 2, 3, 6, or 7”, such reference generally also includes to reference to “embodiment 1-1”, “embodiment 1-2”, “embodiment 1-3”, etc. Likewise, when “Embodiment 8-1” below refers to “embodiment 1, 2, 3, 6, or 7”, same set of embodiments are generally referenced.

When a particular group (e.g. R^{a}, R^{b}, R^{c}, R^{d}, R^{f}, Rⁱ, R^{v}, R^{w}, L, n, q, t, v) appears in a formula more than once, it may assume different identity in each such appearance. For example, in formula -(L)ₜ-(R^{a})ₜ-(L)ₜ-(R^{a})ᵥ—R^{f}, each appearance of L may represent different groups. Likewise, each appearance of R^{a} (including those R^{a} as part of L or R^{f}) may represent different groups; and each appearance oft may represent different numbers. Such expression is not changed by the use of parenthesis. For example, formula —(R^{a}—O)_{q}—R^{f} does not imply that the R^{a} in the parenthesis is identical in each appearance. Rather, it is equivalent to (thus interpreted the same way as) formula —R^{a}—O—R^{a}—O—R^{f} when q is 2, and —R^{a}—O—R^{a}—O—R^{a}—O—R^{f} when q is 3.

Embodiment 1. A compound of Formula Ia:

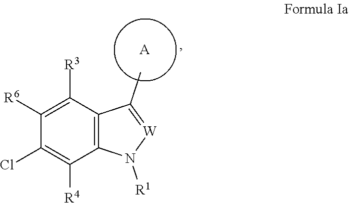

wherein:
ring A is 5-membered heteroaryl containing 1, 2, or 3 nitrogen atoms, wherein the heteroaryl is optionally substituted with 1, 2, or 3 C₁₋₃ alkyl;
X is halo;
W is CR² or N;
R² is H, —C₁₋₃ alkyl, —CR⁵R⁷R⁸, —C(O)—R^{d}, —CH═N—R^{v}, or R^{w};
R¹ is H, —C₁₋₃ alkyl, —R^{a}—C(O)OH, —R^{a}—NH—C(O)CH₃, —R^{a}—NHS(O)₂CH₃, or 5- or 6-membered heteroaryl containing 1, 2, or 3 nitrogen atoms optionally substituted with one or more R^{m};
R⁵ is —R^{a}—OH, —OR^{b}, —N(R^{b})—C(O)—R^{a}—H, —N(R^{b})—C(O)—(R^{a})ₜ—N(R^{b})₂, —N(R^{b})—C(O)—(R^{a})ₜ—N(R^{b})—Rⁱ, —N(R^{b})—C(O)—(R^{a})ₜ—N(R^{b})—(R^{a})ₜ—C(O)—R^{b}, —N(R^{b})—C(O)—R^{d}, —N(R^{b})—R^{a}—R^{d}, —N(R^{b})—(C(O))ₜ—(R^{a})ₜ—Rⁱ, —N(R^{b})—S(O)₂—R^{a}—H, —C(O)—N(R^{b})—R^{a}—Rⁱ, —N(R^{b})—C(S)—R^{a}—H, or —R^{a}—C(O)OH;
or R¹ and R⁵, together with the atoms to which they are immediately attached, form a 5-, 6-, or 7-membered heterocyclic ring containing 1 or 2 nitrogen atoms, wherein the heterocyclic ring is optionally substituted with one or more R^{m};
R³ is H, —YR^{b}, —YR^{c}, —YR^{d}, —(NR^{b})ₙ—R^{a}—H, —Y—R^{a}—CN, —Y—C(O)R^{a}—H, —Y—R^{a}—C(O)N(R^{b})₂, —Y—C(S)R^{a}—H, —Y—C(O)R^{a}—F, —Y—C(O)—R^{a}—CN, or —Y—R^{a}—CH═CH—R^{a}—OH, wherein when R³ is H, then R² is other than H, —C₁₋₃ alkyl, —OH, or —C₁₋₃ alkoxy;
R⁴ is H, halo, or —CN, wherein when R⁴ is H or F, then either (1) R¹ is 5-member heteroaryl containing three nitrogen that is optionally substituted with one of —R^{a}—H, —R^{a}—NH₂, and —R^{a}—C(O)NH₂, or (2) R₃ is —NH—C(O)—CF₂H or —NH—C(O)—CH₂F;
R⁶ is H, halo, or —O—R^{a}—H;
R⁷ and R⁸ are each R^{b}, or R⁷ and R⁸ collectively forms an oxo;
each occurrence of R^{a} is independently —C₁₋₃ alkylene- optionally substituted with one or more substituents selected from the group consisting of halo, —OH, —C₁₋₃ alkyl, —C₁₋₃ alkylene-OH, and —C₁₋₃ alkoxy;
each occurrence of R^{b} is independently —R^{a}—H or —H;
R^{c} is —C₃₋₆ cycloalkyl optionally substituted with one or more —OH, —R^{a}—H, or halo;
R^{d} is 4-8 membered heterocyclyl optionally substituted with oxo, —OH, —C₁₋₃ alkylcarbonyl, or —COOH;
each occurrence of L is selected from the group consisting of —C(O)—, —C(O)—N(R^{b})—, —C(O)—O—, —N(R^{b})—C(O)—, —O—C(O)—, —S(O)₂—, —N(R^{b})—S(O)₂—, and —N(R^{b})C(S)—;
each occurrence of Y is —O— or —N(R^{b})—;
each occurrence of R^{f} is selected from the group consisting of —R^{b}, —Rⁱ, —OR^{b}, —N(R^{b})₂, —(Y)ₜ—(R^{a})ₜ—R^{v}, and -L-R^{b};
each occurrence of Rⁱ is acyl or a 5- to 6-membered heteroaryl containing 1, 2, or 3 heteroatoms independently selected from nitrogen, oxygen, and sulfur and optionally substituted with —C₁₋₃ alkyl;
each occurrence of R^{w} is a 5- or 6-membered heteroaryl, wherein the heteroaryl includes three nitrogen or a combination of two nitrogen with a chalcogen, and wherein the heteroaryl is optionally substituted with —R^{b}, —R^{a}—OH, —CF₃, —N(R^{b})₂, —NHC(O)R^{a}—OH, —SR^{b}, or —R^{c}, wherein when the heteroaryl is a 5-membered heteroaryl with three nitrogen, it is substituted with —SR^{b};
each occurrence of R^{m} is each independently selected from the group consisting of -(L)ₜ-(R^{a})ₜ-(L)ₜ-(R^{a})ᵥ—R^{f}, -(L)ₜ-(R^{a}—O)_{q}—R^{a}—R^{f} and —Y—Rⁱ; or two of R^{m} collectively forms an oxo;
each occurrence of R^{v} is independently a 4-8 membered heterocycle or a 5-6 membered heteroaryl, each optionally substituted with one or more groups selected from C₁₋₃ alkyl, halo, oxo, acyl, —R^{a}—OR^{b}, —NR^{b}₂, —OR^{b}, —C(O)—R^{b}, —C(O)—OR^{b}, —C(O)—R^{d}, C₁₋₃ cycloalkyl, and —SR^{b};
each occurrence of n is independently 1, 2 or 3;
each occurrence oft is independently 0 or 1;
each occurrence of q is independently 1, 2, or 3; and
each occurrence of v is independently 0, 1, or 2,
or a pharmaceutically acceptable salt thereof.
   Embodiment 1-1. The compound of embodiment 1, wherein each occurrence of —N(R^{b})— is —NH—, or a pharmaceutically acceptable salt thereof.
   Embodiment 1-2. The compound of embodiment 1, wherein any halo appearing in R³ is fluoro, or a pharmaceutically acceptable salt thereof.
   Embodiment 1-3. The compound of embodiment 1, 2, 3, 6, or 7, wherein R¹ is selected from the group consisting of: H, —C₁₋₃ alkyl, —R^{a}—C(O)OH, —R^{a}—NH—C(O)CH₃, —R^{a}—NHS(O)₂CH₃,

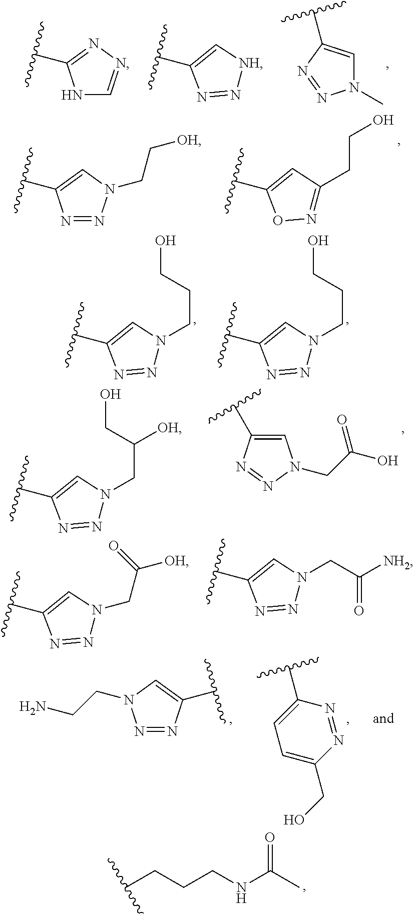

or a pharmaceutically acceptable salt thereof.
Embodiment 2. The compound of embodiment 1, wherein W is CR², or a pharmaceutically acceptable salt thereof.
Embodiment 2-1. The compound of embodiment 1, wherein W is CR², and R² is selected from the group consisting of H, C₁₋₃ alkyl, —C₁₋₃ alkylene-OH, C₁₋₃ alkoxy,

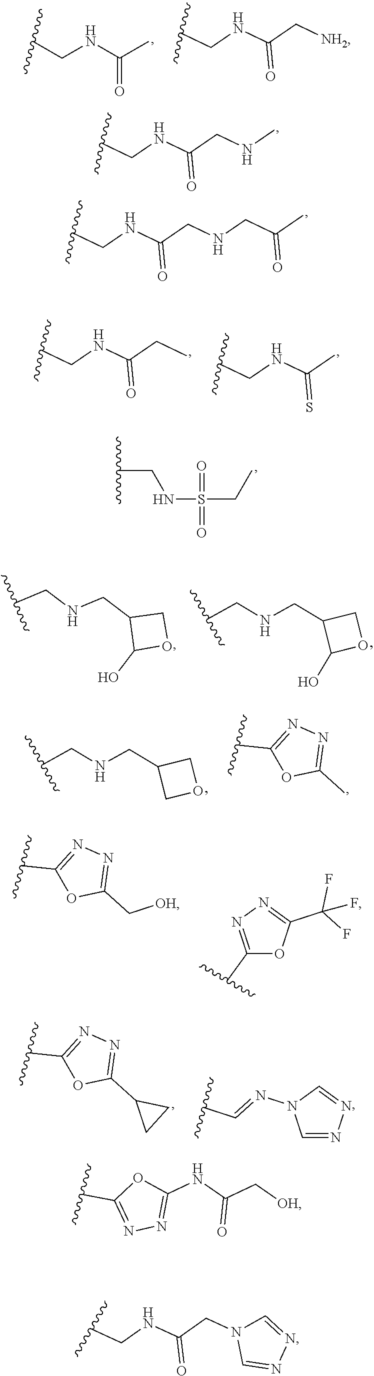

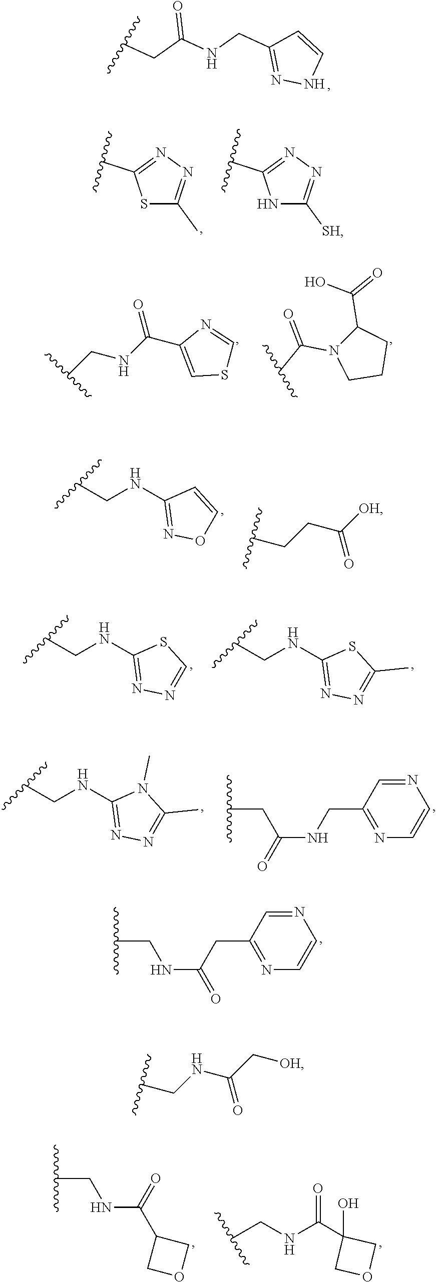

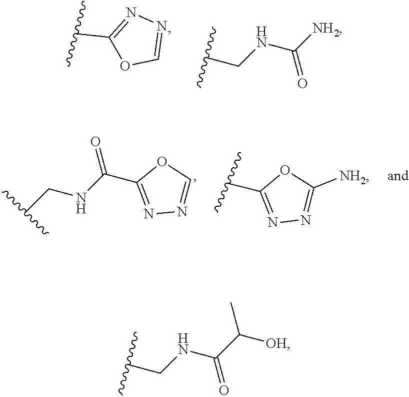

or a pharmaceutically acceptable salt thereof.
Embodiment 2-2. The compound of embodiment 1, wherein W is CR², and R² is selected from the group consisting of H, C₁₋₃ alkyl, —C₁₋₃ alkylene-OH, C₁₋₃ alkoxy,

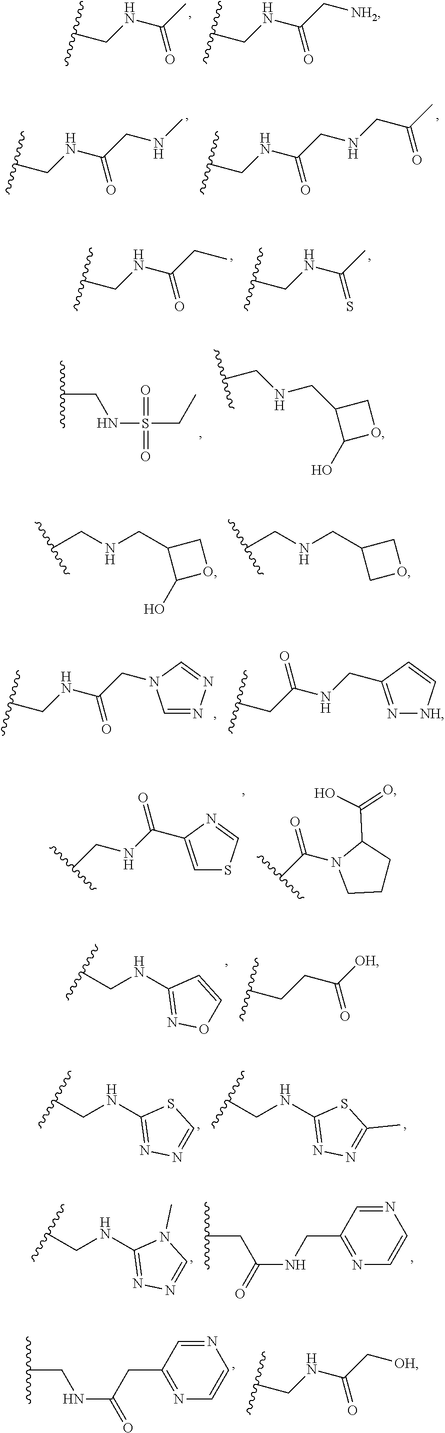

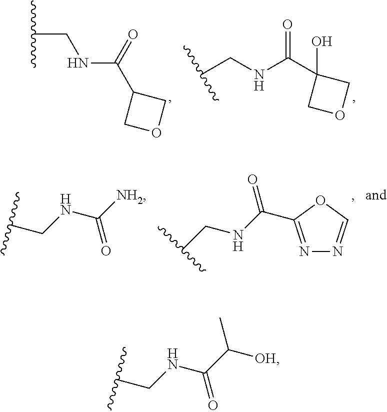

or a pharmaceutically acceptable salt thereof.
Embodiment 3. The compound of embodiment 1 or 2, wherein the compound is of Formula IIIa:

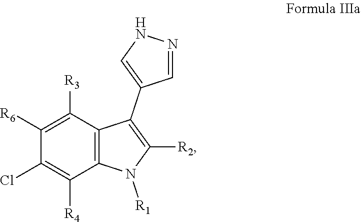

or a pharmaceutically acceptable salt thereof.
Embodiment 4. The compound of embodiment 1, 2, or 3, wherein R² is an optionally substituted 5-membered heteroaryl including two nitrogen and a chalcogen selected from oxygen and sulfur, or a pharmaceutically acceptable salt thereof.
Embodiment 4-1. The compound of embodiment 1, 2, or 3, wherein R² is a 5- or 6-membered heteroaryl, wherein the heteroaryl includes a combination of two nitrogen with a chalcogen selected from oxygen and sulfur, and wherein the heteroaryl is optionally substituted with —R^{b}, —R^{a}—OH, —NHC(O)R^{a}—OH, or —R^{c};
Embodiment 4-2. The compound of embodiment 1, 2, or 3, wherein R² is selected from the group consisting of:

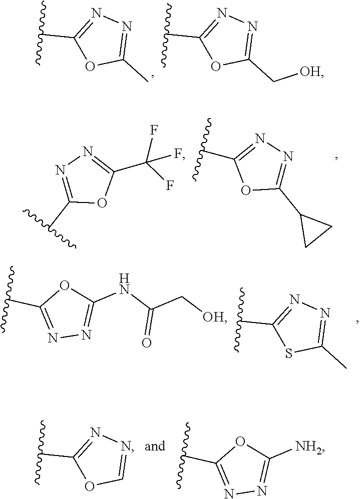

or a pharmaceutically acceptable salt thereof.
Embodiment 4-3. The compound of embodiment 1, 2, or 3, wherein R² is an optionally substituted 6-membered heteroaryl, or a pharmaceutically acceptable salt thereof.
Embodiment 4-4. The compound of embodiment 1, 2, or 3, wherein R² is an optionally substituted 5-membered heteroaryl with three nitrogen and substituted with —SH, or a pharmaceutically acceptable salt thereof.
Embodiment 4-5. The compound of embodiment 1, 2, or 3, wherein R² is

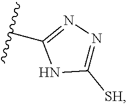 [SMILES: *c1nnc(S)[nH]1]

or a pharmaceutically acceptable salt thereof.
Embodiment 4-6. The compound of embodiment 1, 2, or 3, wherein R² is

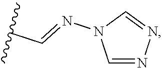 [SMILES: */C=N/n1cnnc1]

or a pharmaceutically acceptable salt thereof.
Embodiment 5. The compound of embodiment 1, 2, or 3, wherein R² is H, —CH₂OH, —CH₂—NH—C(O)—CH₃, or —CH₂—NH—C(O)—CH₂—OH, or a pharmaceutically acceptable salt thereof.
Embodiment 5-1. The compound of embodiment 1, 2, or 3, wherein R² is H or —CH₂NHRⁱ, or a pharmaceutically acceptable salt thereof.
Embodiment 6. The compound of embodiment 1, 2, or 3, wherein R² is —CH₂R⁵ or —C(O)R⁵, or a pharmaceutically acceptable salt thereof.
Embodiment 7. The compound of embodiment 1, having Formula IIa:

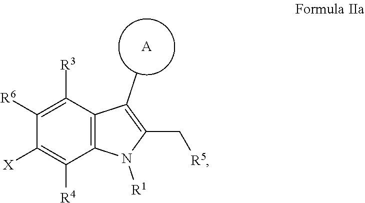

or a pharmaceutically acceptable salt thereof.
Embodiment 8. The compound of embodiment 1, 2, 3, 6, or 7, wherein R⁵ is —OH, —CH₂OH, —C₁₋₃ alkoxy, —NHRⁱ, —NHC(O)Rⁱ, —NHC(O)NH₂, —NH—C(O)—R^{a}—H, —NH—C(S)—R^{a}—H, —C(O)NHCH₂Rⁱ, —NHC(O)CH₂Rⁱ, —NHC(O)CH₂N(R^{b})₂, —NHC(O)CH₂NHC(O)—R^{b}, or —CH₂C(O)OH, or a pharmaceutically acceptable salt thereof.
Embodiment 8-1. The compound of embodiment 1, 2, 3, 6, or 7, wherein R⁵ is —NHRⁱ, or a pharmaceutically acceptable salt thereof.
Embodiment 8-2. The compound of embodiment 1, 2, 3, 6, or 7, wherein R⁵ is —NH—C(O)—CH₃, or a pharmaceutically acceptable salt thereof.
Embodiment 8-3. The compound of embodiment 1, 2, 3, 6, or 7, wherein R⁵ is selected from the group consisting of C₁₋₂ alkyl, —C₁₋₂ alkylene-OH, C₁₋₂ alkoxy,

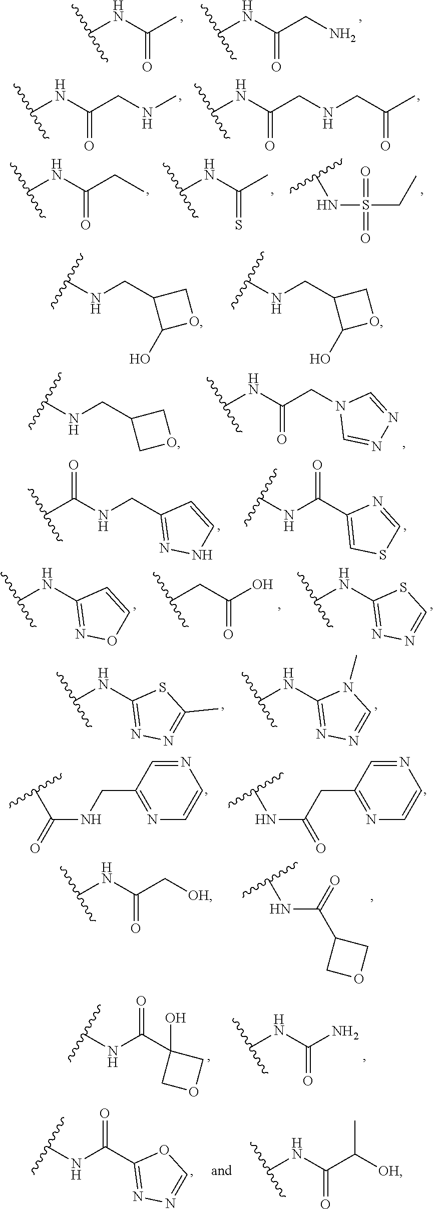

or a pharmaceutically acceptable salt thereof.
Embodiment 9. The compound of embodiment 6 or 7, wherein R¹ and R⁵, together with the atoms to which they are attached, form a 5-, 6-, or 7-membered heterocyclic ring containing 1 or 2 nitrogen atoms, optionally substituted with one or more R^{m}, or a pharmaceutically acceptable salt thereof.
Embodiment 11. The compound of embodiment 1, wherein the compound is of Formula IVa:

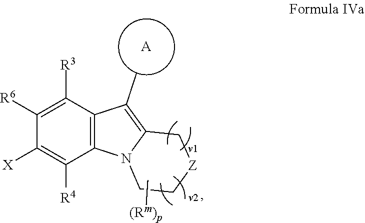

wherein:
Z is CR^{m} or N,
v1 and v2 are each 0, 1, or 2 and v1+v2 is 1, 2, or 3, and
p is 0, 1, 2, 3, or 4, and p is not more than v1+v2+1,
   or a pharmaceutically acceptable salt thereof.
   Embodiment 12. The compound of embodiment 1, wherein the compound is of a formula:

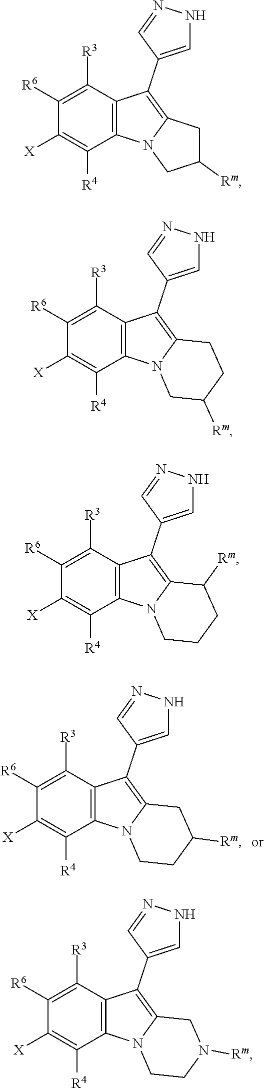

or a pharmaceutically acceptable salt thereof.
Embodiment 13. The compound of embodiment 1, wherein the compound is of a formula:

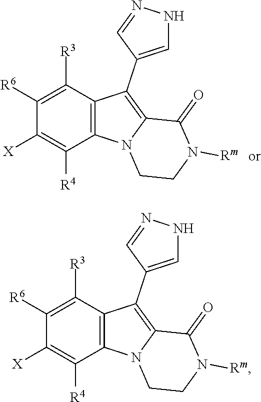

or a pharmaceutically acceptable salt thereof.
Embodiment 14. The compound of embodiment 1, wherein the compound is of Formula IIc:

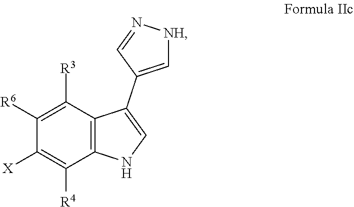

or a pharmaceutically acceptable salt thereof.
Embodiment 14-1. The compound of embodiment 1, wherein the compound is of formula

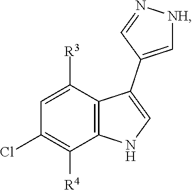

or a pharmaceutically acceptable salt thereof.
Embodiment 15. The compound of embodiment 1, wherein the compound is of Formula V:

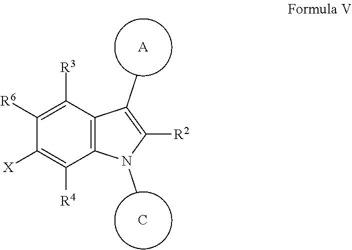

wherein ring C is 5-membered heteroaryl containing 1, 2, or 3 nitrogen and optionally substituted with one or more R^{m}, or a pharmaceutically acceptable salt thereof.
Embodiment 16. The compound of embodiment 1, 2, 3, 4, 5, 6, 7, 8, 9, 11, 12, 13, 14, or 15, wherein R³ is —YR^{b}, —YR^{c}, —YR^{d}, —(NR^{b})ₙ—R^{a}—H, —Y—R^{a}—CN, —Y—C(O)R^{a}—H, —Y—C(S)R^{a}—H, —Y—C(O)R^{a}—F, —Y—C(O)—R^{a}—CN, or —Y—R^{a}—CH═CH—R^{a}—OH, or a pharmaceutically acceptable salt thereof.
Embodiment 16-1. The compound of embodiment 1, 2, 3, 4, 5, 6, 7, 8, 9, 10, 11, 12, 13, 14, or 15, wherein R³ is —YR^{b}, —YR^{c}—YR^{d}, —Y—R^{a}—CN, —Y—C(O)R^{a}—H, —Y—C(S)R^{a}—H, —Y—C(O)R^{a}—F, or —Y—C(O)—R^{a}—CN, or a pharmaceutically acceptable salt thereof.
Embodiment 16-2. The compound of embodiment 1, 2, 3, 4, 5, 6, 7, 8, 9, 10, 11, 12, 13, 14, or 15, wherein R³ attaches to the rest of molecule with N or O, or a pharmaceutically acceptable salt thereof.
Embodiment 16-3. The compound of embodiment 1, 2, 3, 4, 5, 6, or 7, wherein R³ is —YR^{b}, —YR^{c}, —YR^{d}, —(NR^{b})ₙR^{a}—H, —Y—R^{a}—CN, —Y—C(O)R^{a}—H, —Y—C(S)R^{a}—H, —Y—C(O)R^{a}—F, —Y—C(O)—R^{a}—CN, or —Y—R^{a}—CH═CH—R^{a}—OH, or a pharmaceutically acceptable salt thereof.
Embodiment 17. The compound of embodiment 1, 2, 3, 4, 5, 6, 7, 8, 9, 10, 11, 12, 13, 14, or 15, wherein R³ is H, —NH—C(O)—CH₃, —NH—C(O)—CHF₂, —O—(CH₂)₃—OH, —O—CH₂—CN, or —NH—(CH₂)₃—OH, or a pharmaceutically acceptable salt thereof.
Embodiment 18. The compound of embodiment 1, 2, 3, 4, 5, 6, 7, 8, 9, 10, 11, 12, 13, 14, or 15, wherein R³ is
—(NH)ₙCH₃,
—NH—C(O)—R^{a}—CN,
—NH—R^{a}—CN,
—O—R^{a}—CN,
—NH—C(O)—CF₃,
—NHC(S)CH₃,
—NH—R^{a}—C(O)NH₂,
—O—R^{a}—C(O)NH₂,
—NH—R^{a}—CH═CH—R^{a}—OH,
—NH—C(O)—R^{a}—H wherein R^{a} is —C₁₋₃ alkylene- optionally substituted with one or two —OH, one or two F, —C₁₋₃ alkylene-OH, or combinations thereof,
—NH—R^{a}—H wherein R^{a} is —C₁₋₃ alkylene- optionally substituted with one or two —OH, one or two F, —C₁₋₃ alkylene-OH, or combinations thereof,
—O—R^{a}—H wherein R^{a} is —C₁₋₃ alkylene- optionally substituted with one or two —OH, one or two F, —C₁₋₃ alkylene-OH, or combinations thereof,
—OR^{c} wherein R^{c} is —C₃₋₆ cycloalkyl substituted with one or more —OH, —C₁₋₃ alkylene-OH, or halo,
—NHR^{c} wherein R^{c} is —C₃₋₆ cycloalkyl substituted with one or more —OH, —C₁₋₃ alkylene-OH, or halo,
—NHR^{d}, or
—OR^{d},
   or a pharmaceutically acceptable salt thereof.
   Embodiment 18-1. The compound of embodiment 1, 2, 3, 4, 5, 6, 7, 8, 9, 10, 11, 12, 13, 14, or 15, wherein R³ is
—NH—C(O)—CH₂—CN,
—NH—CH₂—CN,
—O—CH₂—CN,
—NH—C(O)—CF₃,
—NH—C(O)—R^{a}—H wherein R^{a} is —C₁₋₃ alkylene- optionally substituted with one or two —OH, one or two F, or combinations thereof,
—NH—R^{a}—H wherein R^{a} is —C₁₋₃ alkylene- optionally substituted with one or two —OH, one or two F, or combinations thereof,
—O—R^{a}—H wherein R^{a} is —C₁₋₃ alkylene- optionally substituted with one or two —OH, one or two F, or combinations thereof,
—NHC(S)CH₃,
—NH—CH₂—C(O)NH₂,
—O—CH₂—C(O)NH₂,
—NHR^{d}
—OR^{d},
—OR^{c} wherein R^{c} is —C₃₋₆ cycloalkyl substituted with —OH, —CH₂OH or F, or
—NHR^{c} wherein R^{c} is —C₃₋₆ cycloalkyl substituted with —OH, —CH₂OH or F, or a pharmaceutically acceptable salt thereof.
   Embodiment 18-2. The compound of embodiment 1, 2, 3, 4, 5, 6, 7, 8, 9, 10, 11, 12, 13, 14, or 15, wherein R³ is —NH—C(O)—R^{a}—H or —O—R^{a}—CN, or a pharmaceutically acceptable salt thereof.
   Embodiment 18-3. The compound of embodiment 1, 2, 3, 4, 5, 6, 7, 8, 9, 10, 11, 12, 13, 14, or 15, wherein R³ is —NHC(O)CH₃ or —OCH₂—CN, or a pharmaceutically acceptable salt thereof.
   Embodiment 18-4. The compound of embodiment 1, 2, 3, 4, 5, 6, 7, 8, 9, 10, 11, 12, 13, 14, or 15, wherein R³ is selected from the group consisting of: H, C₁₋₃ alkoxy,

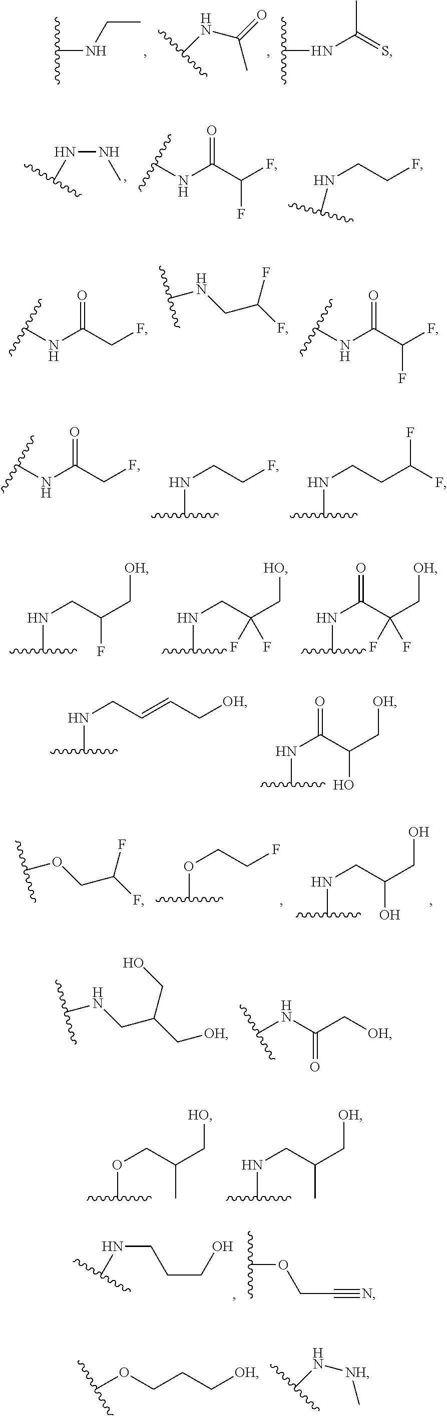

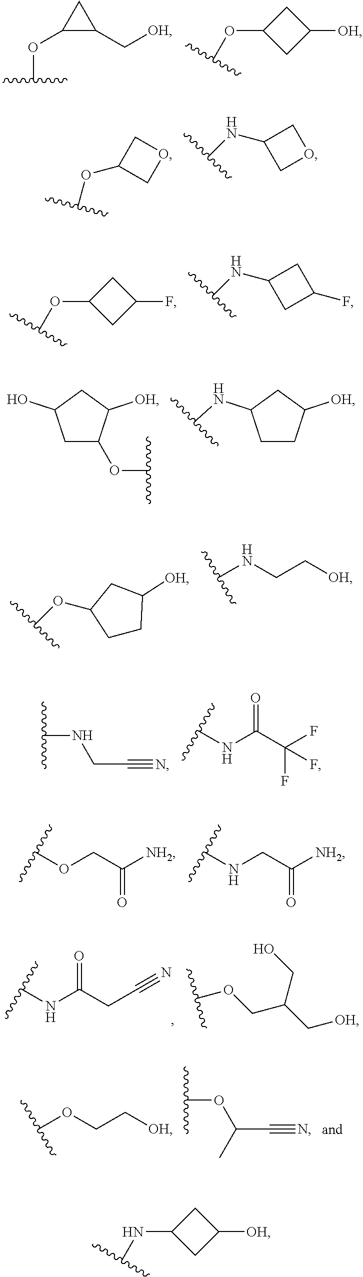

or a pharmaceutically acceptable salt thereof.
Embodiment 18-5. A compound of Formula Ia:

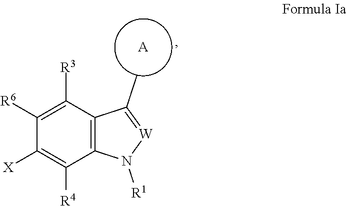

wherein:
ring A is 5-membered heteroaryl containing 1, 2, or 3 nitrogen atoms, wherein the heteroaryl is optionally substituted with 1, 2, or 3 C₁₋₃ alkyl;
X is halo;
W is CR² or N;
R² is H, —C₁₋₃ alkyl, —CR⁵R⁷R⁸, —C(O)—R^{d}, —CH═N—R^{v}, or R^{w};
R¹ is H, —C₁₋₃ alkyl, —R^{a}—C(O)OH, —R^{a}—NH—C(O)CH₃, —R^{a}—NHS(O)₂CH₃, or 5- or 6-membered heteroaryl containing 1, 2, or 3 nitrogen atoms optionally substituted with one or more R^{m};
R⁵ is —R^{a}—OH, —OR^{b}, —N(R^{b})—C(O)—R^{a}—H, —N(R^{b})—C(O)—(R^{a})ₜ—N(R^{b})₂, —N(R^{b})—C(O)—(R^{a})ₜ—N(R^{b})—Rⁱ, —N(R^{b})—C(O)—(R^{a})ₜ—N(R^{b})—(R^{a})ₜ—C(O)—R^{b}, —N(R^{b})—C(O)—R^{d}, —N(R^{b})—R^{a}—R^{d}, —N(R^{b})—(C(O))ₜ—(R^{a})ₜ—Rⁱ, —N(R^{b})—S(O)₂—R^{a}—H, —C(O)—N(R^{b})—R^{a}—Rⁱ, —N(R^{b})—C(S)—R^{a}—H, or —R^{a}—C(O)OH;
or R¹ and R⁵, together with the atoms to which they are immediately attached, form a 5-, 6-, or 7-membered heterocyclic ring containing 1 or 2 nitrogen atoms, wherein the heterocyclic ring is optionally substituted with one or more R^{m};
R³ is selected from the group consisting of: H, C₁₋₃ alkoxy,

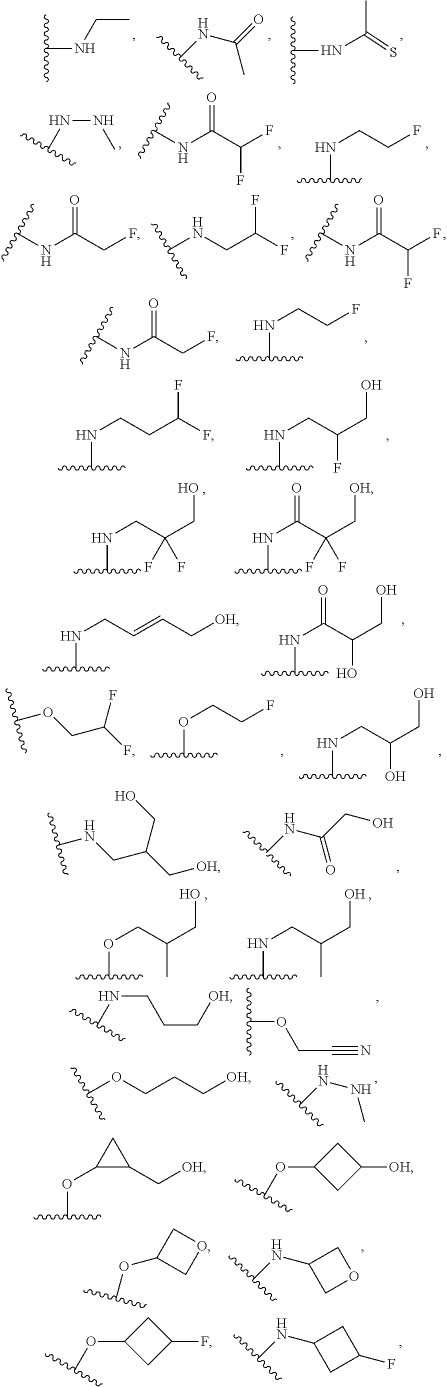

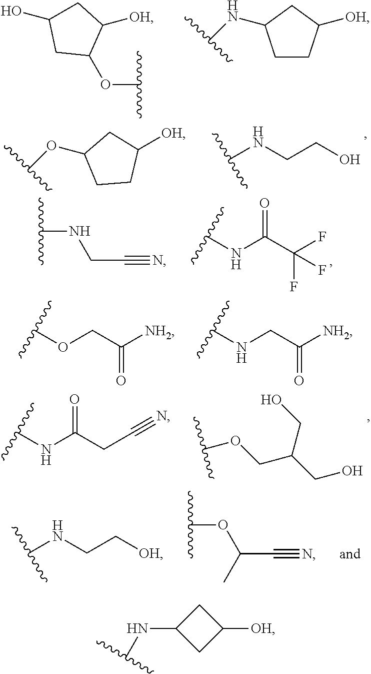

R⁴ is H, halo, or —CN, wherein when R⁴ is H or F, then either (1) R¹ is 5-member heteroaryl containing three nitrogen that is optionally substituted with one of —R^{a}—H, —R^{a}—NH₂, and —R^{a}—C(O)NH₂, or (2) R₃ is —NH—C(O)—CF₂H or —NH—C(O)—CH₂F;
R⁶ is H, halo, or —O—R^{a}—H;
R⁷ and R⁸ are each R^{b}, or R⁷ and R⁸ collectively forms an oxo;
each occurrence of R^{a} is independently —C₁₋₃ alkylene- optionally substituted with one or more substituents selected from the group consisting of halo, —OH, —C₁₋₃ alkyl, —C₁₋₃ alkylene-OH, and —C₁₋₃ alkoxy;
each occurrence of R^{b} is independently —R^{a}—H or —H;
R^{c} is —C₃₋₆ cycloalkyl optionally substituted with one or more —OH, —R^{a}—H, or halo;
R^{d} is 4-8 membered heterocyclyl optionally substituted with oxo, —OH, —C₁₋₃ alkylcarbonyl, or —COOH;
each occurrence of L is selected from the group consisting of —C(O)—, —C(O)—N(R^{b})—, —C(O)—O—, —N(R^{b})—C(O)—, —O—C(O)—, —S(O)₂—, —N(R^{b})—S(O)₂—, and —N(R^{b})C(S)—;
each occurrence of Y is —O— or —N(R^{b})—;
each occurrence of R^{f} is selected from the group consisting of —R^{b}, —Rⁱ, —OR^{b}, —N(R^{b})₂, —(Y)ₜ—(R^{a})ₜ—R^{v}, and -L-R^{b};
each occurrence of Rⁱ is acyl or a 5- to 6-membered heteroaryl containing 1, 2, or 3 heteroatoms independently selected from nitrogen, oxygen, and sulfur and optionally substituted with —C₁₋₃ alkyl;
each occurrence of R^{w} is a 5- or 6-membered heteroaryl, wherein the heteroaryl includes three nitrogen or a combination of two nitrogen with a chalcogen, and wherein the heteroaryl is optionally substituted with —R^{b}, —R^{a}—OH, —CF₃, —N(R^{b})₂, —NHC(O)R^{a}—OH, —SR^{b}, or —R^{c}, wherein when the heteroaryl is a 5-membered heteroaryl with three nitrogen, it is substituted with —SR^{b};
each occurrence of R^{m} is each independently selected from the group consisting of -(L)ₜ-(R^{a})ₜ-(L)ₜ-(R^{a})ᵥ—R^{f}, -(L)ₜ-(R^{a}—O)_{q}—R^{a}—R^{f} and —Y—Rⁱ; or two of R^{m} collectively forms an oxo;
each occurrence of R^{v} is independently a 4-8 membered heterocycle or a 5-6 membered heteroaryl, each optionally substituted with one or more groups selected from C₁₋₃ alkyl, halo, oxo, acyl, —R^{a}—OR^{b}, —NR^{b}₂, —OR^{b}, —C(O)—R^{b}, —C(O)—OR^{b}, —C(O)—R^{d}, C₁₋₃ cycloalkyl, and —SR^{b};
each occurrence of n is independently 1, 2 or 3;
each occurrence oft is independently 0 or 1;
each occurrence of q is independently 1, 2, or 3; and
each occurrence of v is independently 0, 1, or 2,
or a pharmaceutically acceptable salt thereof.
   Embodiment 19. The compound of embodiment 1, 2, 3, 4, 5, 6, 7, 8, 9, 10, 11, 12, 13, 14, or 15, wherein R³ is H, or a pharmaceutically acceptable salt thereof.
   Embodiment 20. The compound of embodiment 1, 2, 3, 4, 5, 6, 7, 8, 9, 10, 11, 12, 13, 14, 15, 16, 17, 18, or 19, wherein R⁴ is Cl, or a pharmaceutically acceptable salt thereof.
   Embodiment 20-1. The compound of embodiment 1, 2, 3, 4, 5, 6, 7, 8, 9, 10, 11, 12, 13, 14, 15, 16, 17, 18, or 19, having formula:

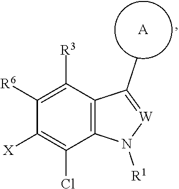

or a pharmaceutically acceptable salt thereof.
Embodiment 20-2. The compound of embodiment 1, 2, 3, 4, 5, 6, 7, 8, 9, 10, 11, 12, 13, 14, 15, 16, 17, 18, or 19, having formula:

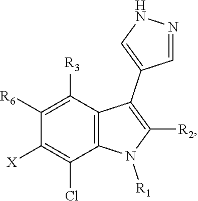

or a pharmaceutically acceptable salt thereof.
Embodiment 20-3. The compound of embodiment 1, 2, 3, 6, 7, 8, 9, 10, 11, 12, 13, 14, 15, 16, 17, 18, or 19, having formula:

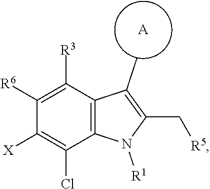

or a pharmaceutically acceptable salt thereof.
Embodiment 20-4. The compound of embodiment 1, 2, 3, 6, 7, 8, 9, 10, 11, 12, 13, 14, 15, 16, 17, 18, or 19, having formula:

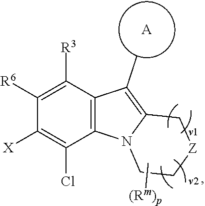

or a pharmaceutically acceptable salt thereof.
Embodiment 20-5. The compound of embodiment 1, 2, 3, 6, 7, 8, 9, 10, 11, 12, 13, 14, 15, 16, 17, 18, or 19, having formula:

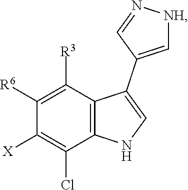

or a pharmaceutically acceptable salt thereof.
Embodiment 21. The compound of embodiment 1, 2, 3, 4, 5, 6, 7, 8, 15, 16, 17, 18, or 19, wherein R⁴ is F; and R¹ is 5-member heteroaryl containing three nitrogen that is optionally substituted with —R^{a}—H, or a pharmaceutically acceptable salt thereof.
Embodiment 22. The compound of embodiment 1, 2, 3, 4, 5, 6, 7, 8, 15, 16, 17, 18, or 19, wherein R⁴ is H; and R¹ is 5-member heteroaryl containing three nitrogen that is optionally substituted with —R^{a}—H, —R^{a}—NH₂, or —R^{a}—C(O)NH₂, or a pharmaceutically acceptable salt thereof.
Embodiment 23. The compound of embodiment 1, 2, 3, 4, 5, 6, 7, 8, 9, 10, 11, 12, 13, 14, 15, 16, 17, 18, 19, 20, 21, or 22, wherein R⁶ is H, or a pharmaceutically acceptable salt thereof.
Embodiment 23-1. The compound of embodiment 1, 2, 3, 4, 5, 6, 7, 8, 9, 10, 11, 12, 13, 14, 15, 16, 17, 18, 19, or 20, having formula:

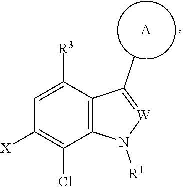

or a pharmaceutically acceptable salt thereof.
Embodiment 23-2. The compound of embodiment 1, 2, 3, 4, 5, 6, 7, 8, 9, 10, 11, 12, 13, 14, 15, 16, 17, 18, 19, or 20, having formula:

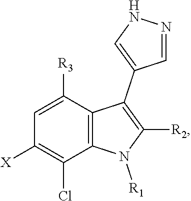

or a pharmaceutically acceptable salt thereof.
Embodiment 23-3. The compound of embodiment 1, 2, 3, 6, 7, 8, 9, 10, 11, 12, 13, 14, 15, 16, 17, 18, 19, or 20, having formula:

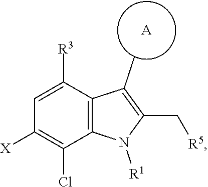

or a pharmaceutically acceptable salt thereof.
Embodiment 23-4. The compound of embodiment 1, 2, 3, 6, 7, 8, 9, 10, 11, 12, 13, 14, 15, 16, 17, 18, 19, or 20, having formula:

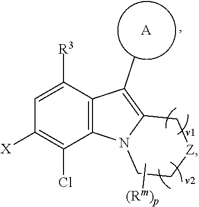

or a pharmaceutically acceptable salt thereof.
Embodiment 23-5. The compound of embodiment 1, 2, 3, 6, 7, 8, 9, 10, 11, 12, 13, 14, 15, 16, 17, 18, 19, or 20, having formula:

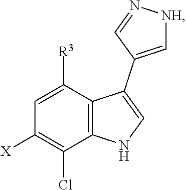

or a pharmaceutically acceptable salt thereof.
Embodiment 24. The compound of embodiment 1, 2, 3, 4, 5, 6, 7, 8, 9, 10, 11, 12, 13, 14, 15, 16, 17, 18, or 19, wherein R⁶ is —O—R^{a}—H, and R⁴ is H, or a pharmaceutically acceptable salt thereof.
Embodiment 26. The compound of embodiment 1, wherein W is N; R³ is —O—R^{a}—CN, —NH—R^{a}—H, or —NH—C(O)—R^{a}—H, wherein R^{a} is —C₁₋₃ alkylene- optionally with one or two —OH, one or two F, or combinations thereof, or a pharmaceutically acceptable salt thereof.
Embodiment 26-1. The compound of embodiment 1, wherein W is N; R³ is —O—CH₂—CN, —NH—(CH₂)₃—H, or —NH—C(O)—CH₂OH, or —NH—C(O)—CHF₂, or a pharmaceutically acceptable salt thereof.
Embodiment 27. The compound of embodiment 1, 2, 3, 4, 5, 6, 7, 8, 9, 10, 11, 12, 13, 16, 17, 18, 19, 20, 21, 22, 23, 24, 25, or 26, wherein R^{m} is each independently selected from H, —OH, —NH₂, —R^{a}—H, —R^{v}, —R^{a}—R^{v}, —C(O)R^{a}—H, —C(O)R^{v}, —C(O)—R^{a}—R^{v}, —R^{a}—NH₂, —C(O)NHCH₃, —NHC(O)—R^{a}—H, —NHC(O)—R^{a}—NHC(O)R^{b}, —NHC(O)R^{d}, —NHC(O)R^{a}—OR^{b}, —NHC(O)—(R^{a}—O)_{q}—R^{a}—NH₂, —NHC(O)—R^{a}—NH—C(O)—R^{a}—H, —NHC(O)—R^{a}—C(O)—NR^{b}₂, —NHC(O)—R^{a}—NH—C(O)—(R^{a})ᵥ—NH₂, —(R^{a}—O)_{q}—R^{a}—H, —R^{a}—OH, —R^{a}—O—R^{a}—H, —C(O)OH, —CH₂C(O)OH, —CH₂CONH₂, —C(O)R^{a}—H, —C(O)—R^{a}—OR^{b}, —C(O)—(R^{a}—O)—(R^{a}—O)—R^{b}, —C(O)—R^{a}—N(R^{b})—C(O)R^{a}—OR^{b}, —C(O)—(R^{a}—O)—R^{a}—R^{v}, —C(O)—R^{a}—N(R^{b})C(O)R^{a}—H, —C(O)—R^{a}—N(R^{b})C(O)R^{a}—OR^{b}, —C(O)—R^{a}—N(R^{b})C(O)R^{a}—N(R^{b})₂, —C(O)—R^{a}—O—R^{a}—C(O)N(R^{b})₂, —C(O)—CH(—C₁₋₃ alkoxy)-R^{a}—OR^{b}, —C(O)R^{a}—C(O)N(R^{b})₂, —C(O)—R^{a}—N(R^{b})C(O)—R^{a}—H, —S(O)₂NH₂, —S(O)₂CH₃, —NHS(O)₂CH₃, —NHS(O)₂R^{a}—R^{v}, —NHS(O)₂R^{a}—NH—C(O)—R^{a}—H, —NHS(O)₂R^{a}—C(O)—NH—R^{a}—H, and —NHS(O)₂R^{a}—O—R^{a}—H; or
two of R^{m} collectively forms an oxo;
or a pharmaceutically acceptable salt thereof.
   Embodiment 28. The compound of embodiment 1, 2, 3, 4, 5, 6, 7, 8, 9, 10, 11, 12, 13, 16, 17, 18, 19, 20, 21, 22, 23, 24, 25, or 26, wherein R^{m} is each independently selected from the group consisting of: H, C₁₋₃ alkyl, —OH, —C₁₋₃ alkylene-OH, —C₁₋₃ alkylene-NH₂, C₁₋₃ alkoxy, —C(O)—OH,

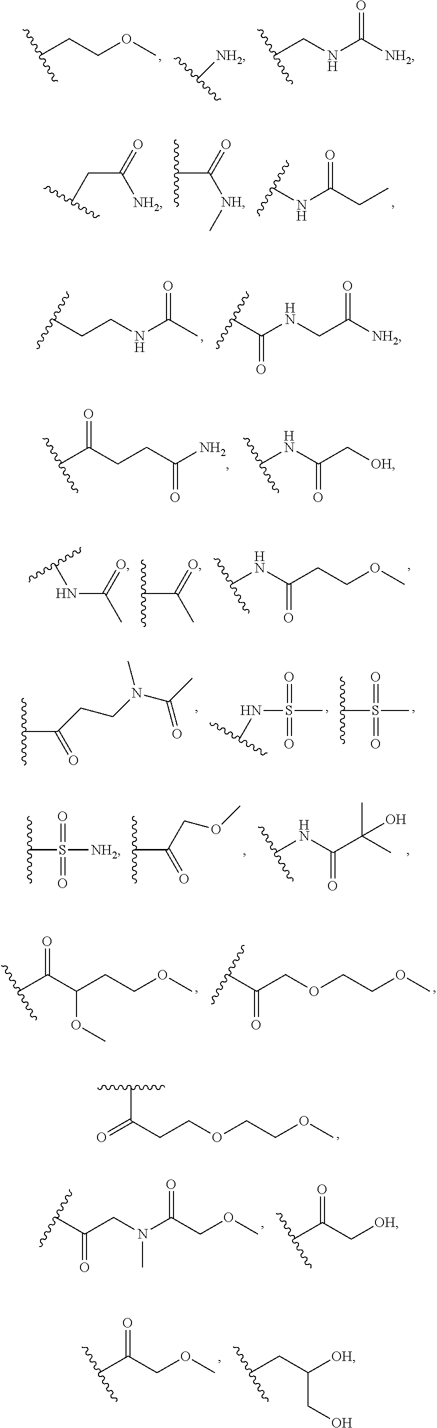

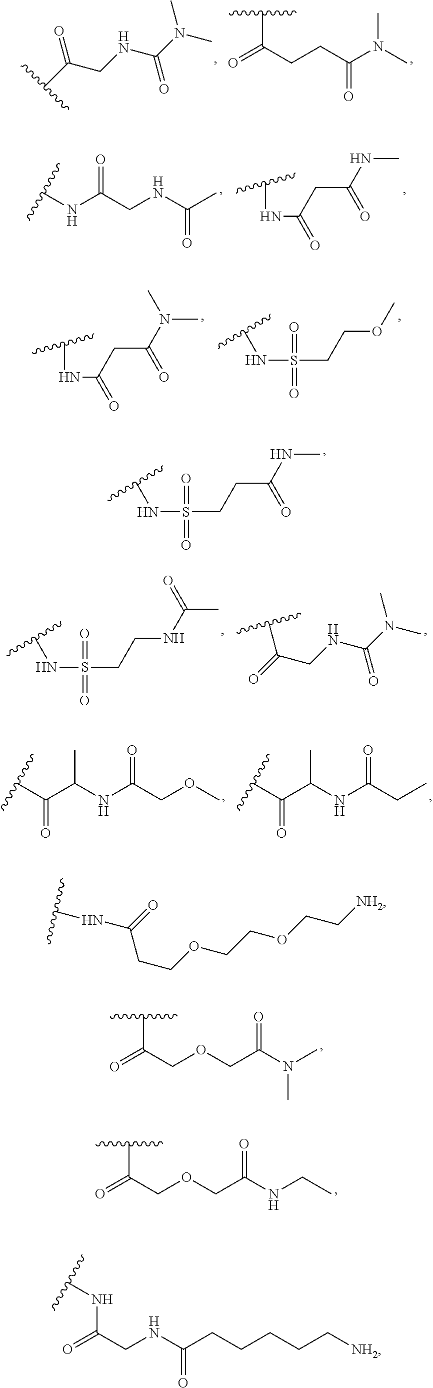

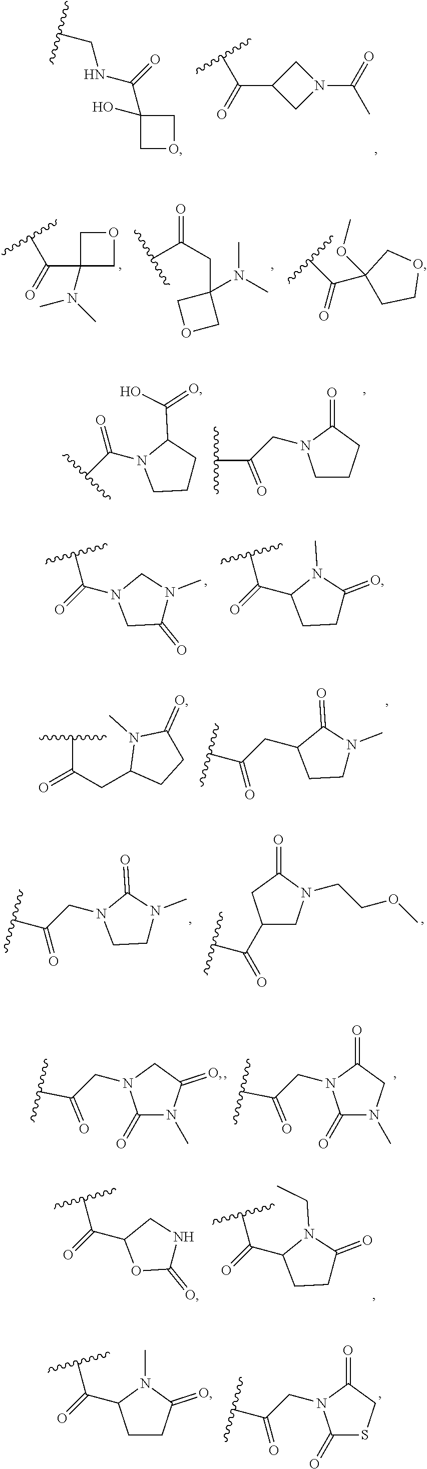

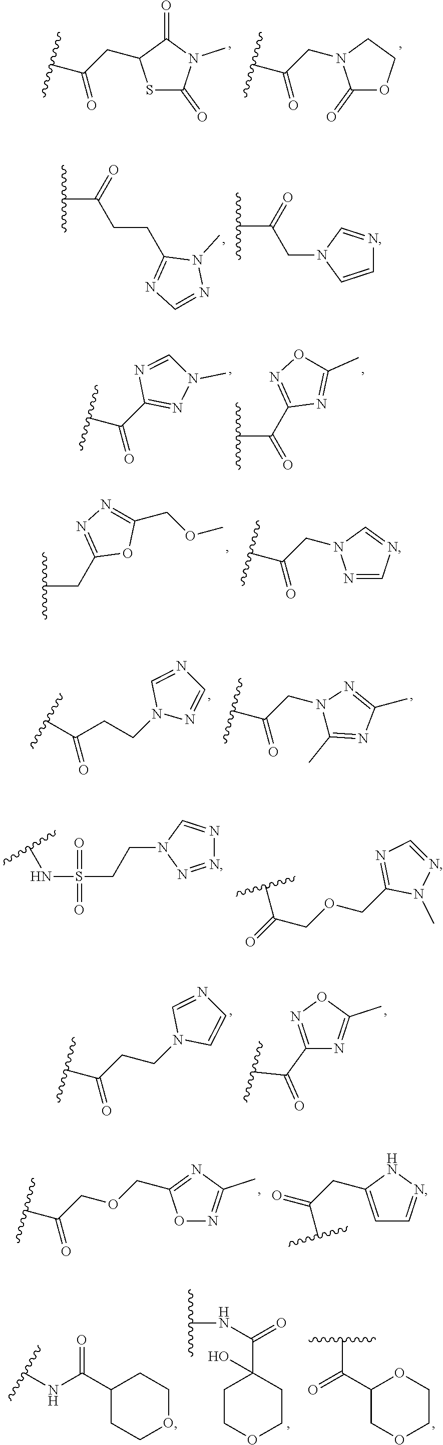

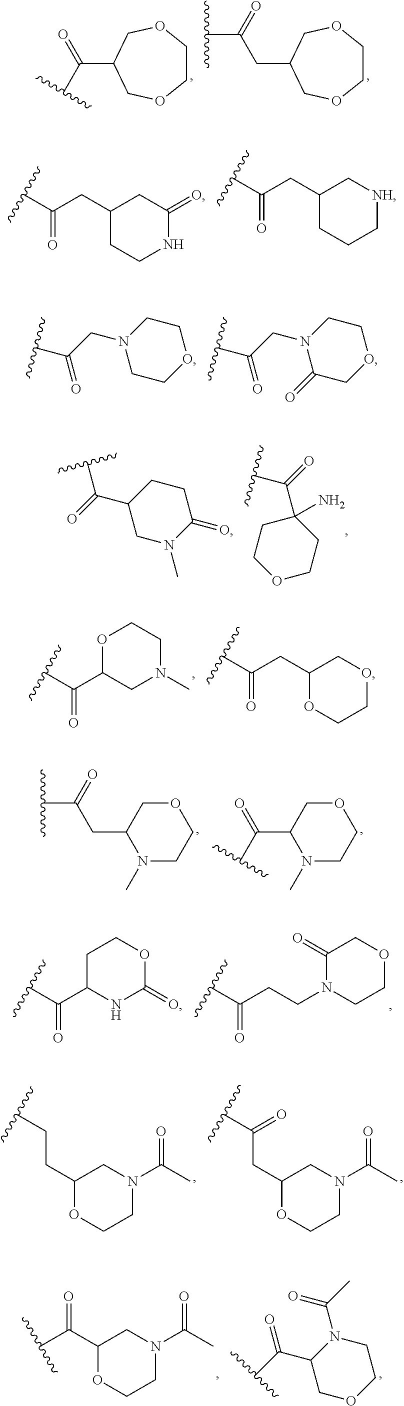

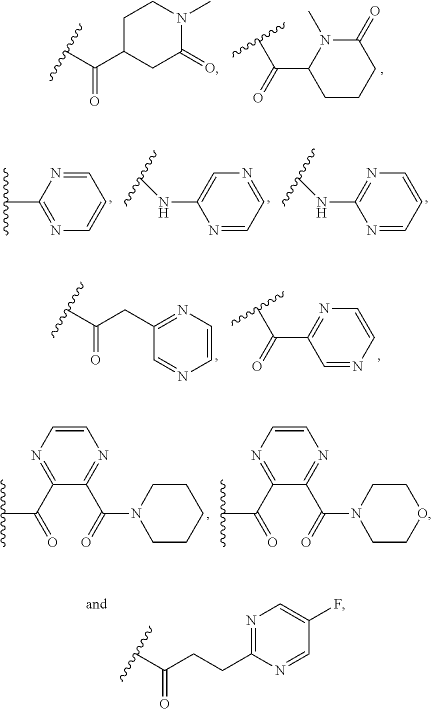

or two R^{m} collectively forms an oxo,
or a pharmaceutically acceptable salt thereof.
Embodiment 29. The compound of embodiment 1, 2, 3, 4, 5, 6, 7, 8, 16, 17, 18, 19, 20, 21, 22, 23, 24, 25, or 26, wherein R¹ is H or CH₃, or a pharmaceutically acceptable salt thereof.
Embodiment 30. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, or 29, wherein X is Cl, or a pharmaceutically acceptable salt thereof.
Embodiment 30-1. The compound of embodiment 1, 2, 3, 4, 5, 6, 7, 8, 9, 10, 11, 12, 13, 14, 15, 16, 17, 18, 19, 20, 23, 26, 27, 28, or 29, having formula:

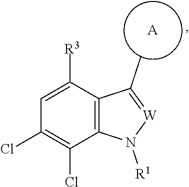

or a pharmaceutically acceptable salt thereof.
Embodiment 30-2. The compound of embodiment 1, 2, 3, 4, 5, 6, 7, 8, 9, 10, 11, 12, 13, 14, 15, 16, 17, 18, 19, 20, 23, 27, 28, or 29, having formula:

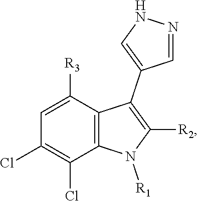

or a pharmaceutically acceptable salt thereof.
Embodiment 30-3. The compound of embodiment 1, 2, 3, 6, 7, 8, 9, 10, 11, 12, 13, 14, 15, 16, 17, 18, 19, 20, 23, 27, 28, or 29, having formula:

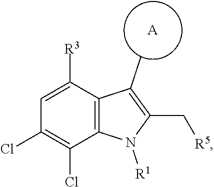

or a pharmaceutically acceptable salt thereof.
Embodiment 30-4. The compound of embodiment 1, 2, 3, 6, 7, 8, 9, 10, 11, 12, 13, 14, 15, 16, 17, 18, 19, 20, 23, 27, 28, or 29, having formula:

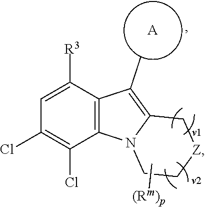

or a pharmaceutically acceptable salt thereof.
Embodiment 30-5. The compound of embodiment 1, 2, 3, 6, 7, 8, 9, 10, 11, 12, 13, 14, 15, 16, 17, 18, 19, 20, 23, 27, or 28, having formula:

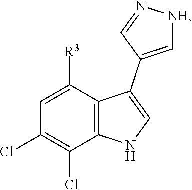

or a pharmaceutically acceptable salt thereof.
Embodiment 31. The compound of embodiment 1, wherein:
R⁴ is Cl;
X is Cl;
R⁶ is H;
W is CR²;
R² is H, —C₁₋₃ alkyl, —CR⁵R⁷R⁸, —C(O)—R^{d}, or R^{w};
R¹ is H, C₁₋₃ alkyl, —R^{a}—C(O)OH, —R^{a}—NH—C(O)CH₃, —R^{a}—NHS(O)₂CH₃, or 5-membered heteroaryl containing 1, 2, or 3 nitrogen atoms optionally substituted with one or more R^{m};
R⁵ is —R^{a}—OH, —OR^{b}, —N(R^{b})—C(O)—R^{a}—H, —N(R^{b})—C(O)—(R^{a})ₜ—N(R^{b})₂, —N(R^{b})—C(O)—(R^{a})ₜ—N(R^{b})—Rⁱ, —N(R^{b})—C(O)—(R^{a})ₜ—N(R^{b})—(R^{a})ₜ—C(O)—R^{b}, —N(R^{b})—C(O)—R^{d}, —N(R^{b})—R^{a}—R^{d}, —N(R^{b})—(C(O))ₜ—(R^{a})ₜ—Rⁱ, —C(O)—N(R^{b})—R^{a}—Rⁱ, or —R^{a}—C(O)OH;
or R¹ and R⁵, together with the atoms to which they are immediately attached, form a 5-, 6-, or 7-membered heterocyclic ring containing 1 or 2 nitrogen atoms, wherein the heterocyclic ring is optionally substituted with one or more R^{m};
R³ is H, —YR^{b}, —YR^{c}, —YR^{d}—(NR^{b})ₙ—R^{a}—H, —Y—R^{a}—CN, —Y—C(O)R^{a}—H, —Y—R^{a}—C(O)N(R^{b})₂, —Y—C(S)R^{a}—H, —Y—C(O)R^{a}—F, or —Y—C(O)—R^{a}—CN, wherein when R³ is —H, then R² is other than H, —C₁₋₃ alkyl, —OH, or —C₁₋₃ alkoxy; and
ring A is

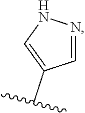 [SMILES: *c1cn[nH]c1]

or a pharmaceutically acceptable salt thereof.
   Embodiment 31-1. The compound of embodiment 1, wherein:
R⁴ is Cl;
X is Cl;
R⁶ is H;
W is CR²;
R² is H, —C₁₋₃ alkyl, —CR⁵R⁷R⁸, —C(O)—R^{d}, or R^{w};
R¹ is H, C₁₋₃ alkyl, —R^{a}—C(O)OH, —R^{a}—NH—C(O)CH₃, —R^{a}—NHS(O)₂CH₃, or 5-membered heteroaryl containing 1, 2, or 3 nitrogen atoms optionally substituted with one or more R^{m};
R⁵ is —R^{a}—OH, —OR^{b}, —N(R^{b})—C(O)—R^{a}—H, —N(R^{b})—C(O)—(R^{a})ₜ—N(R^{b})₂, —N(R^{b})—C(O)—(R^{a})ₜ—N(R^{b})—Rⁱ, —N(R^{b})—C(O)—(R^{a})ₜ—N(R^{b})—(R^{a})ₜ—C(O)—R^{b}, —N(R^{b})—C(O)—R^{d}, —N(R^{b})—R^{a}—R^{d}, —N(R^{b})—(C(O))ₜ—(R^{a})ₜ—Rⁱ, —C(O)—N(R^{b})—R^{a}—Rⁱ, or —R^{a}—C(O)OH;
or R¹ and R⁵, together with the atoms to which they are immediately attached, form a 5-, 6-, or 7-membered heterocyclic ring containing 1 or 2 nitrogen atoms, wherein the heterocyclic ring is optionally substituted with one or more R^{m};
R³ is —YR^{b}, —YR^{c}, —YR^{d}, —(NR^{b})ₙ—R^{a}—H, —Y—R^{a}—CN, —Y—C(O)R^{a}—H, —Y—R^{a}—C(O)N(R^{b})₂, —Y—C(S)R^{a}—H, —Y—C(O)R^{a}—F, or —Y—C(O)—R^{a}—CN; and
ring A is

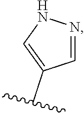 [SMILES: *c1cn[nH]c1]

or a pharmaceutically acceptable salt thereof.
   Embodiment 31-2. A compound having formula:

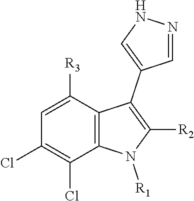

wherein:
R² is H, —C₁₋₃ alkyl, —CR⁵R⁷R⁸, —C(O)—R^{d}, —CH═N—R^{v}, or R^{w};
R¹ is H, —C₁₋₃ alkyl, —R^{a}—C(O)OH, —R^{a}—NH—C(O)CH₃, —R^{a}—NHS(O)₂CH₃, or 5- or 6-membered heteroaryl containing 1, 2, or 3 nitrogen atoms optionally substituted with one or more R^{m};
R⁵ is —R^{a}—OH, —OR^{b}, —N(R^{b})—C(O)—R^{a}—H, —N(R^{b})—C(O)—(R^{a})ₜ—N(R^{b})₂, —N(R^{b})—C(O)—(R^{a})ₜ—N(R^{b})—Rⁱ, —N(R^{b})—C(O)—(R^{a})ₜ—N(R^{b})—(R^{a})ₜ—C(O)—R^{b}, —N(R^{b})—C(O)—R^{d}, —N(R^{b})—R^{a}—R^{d}, —N(R^{b})—(C(O))ₜ—(Rₐ)ₜ—Rⁱ, —N(R^{b})—S(O)₂—R^{a}—H, —C(O)—N(R^{b})—R^{a}—Rⁱ, —N(R^{b})—C(S)—R^{a}—H, or —R^{a}—C(O)OH;
or R¹ and R⁵, together with the atoms to which they are immediately attached, form a 5-, 6-, or 7-membered heterocyclic ring containing 1 or 2 nitrogen atoms, wherein the heterocyclic ring is optionally substituted with one or more R^{m};
R³ is H, —YR^{b}, —YR^{c}, —YR^{d}, —(NR^{b})ₙ—R^{a}—H, —Y—R^{a}—CN, —Y—C(O)R^{a}—H, —Y—R^{a}—C(O)N(R^{b})₂, —Y—C(S)R^{a}—H, —Y—C(O)R^{a}—F, —Y—C(O)—R^{a}—CN, or —Y—R^{a}—CH═CH—R^{a}—OH, wherein when R³ is H, then R² is other than H, —C₁₋₃ alkyl, —OH, or —C₁₋₃ alkoxy;
R⁷ and R⁸ are each R^{b}, or R⁷ and R⁸ collectively forms an oxo;
each occurrence of R^{a} is independently —C₁₋₃ alkylene- optionally substituted with one or more substituents selected from the group consisting of halo, —OH, —C₁₋₃ alkyl, —C₁₋₃ alkylene-OH, and —C₁₋₃ alkoxy;
each occurrence of R^{b} is independently —R^{a}—H or —H;
R^{c} is —C₃₋₆ cycloalkyl optionally substituted with one or more —OH, —R^{a}—H, or halo;
R^{d} is 4-8 membered heterocyclyl optionally substituted with oxo, —OH, —C₁₋₃ alkylcarbonyl, or —COOH;
each occurrence of L is selected from the group consisting of —C(O)—, —C(O)—N(R^{b})—, —C(O)—O—, —N(R^{b})—C(O)—, —O—C(O)—, —S(O)₂—, —N(R^{b})—S(O)₂—, and —N(R^{b})C(S)—;
each occurrence of Y is —O— or —N(R^{b})—;
each occurrence of R^{f} is selected from the group consisting of —R^{b}, —Rⁱ, —OR^{b}, —N(R^{b})₂, —(Y)ₜ—(R^{a})ₜ—R^{v}, and -L-R^{b};
each occurrence of Rⁱ is acyl or a 5- to 6-membered heteroaryl containing 1, 2, or 3 heteroatoms independently selected from nitrogen, oxygen, and sulfur and optionally substituted with —C₁₋₃ alkyl;
each occurrence of R_{w} is a 5- or 6-membered heteroaryl, wherein the heteroaryl includes three nitrogen or a combination of two nitrogen with a chalcogen, and wherein the heteroaryl is optionally substituted with —R^{b}, —R^{a}—OH, —CF₃, —N(R^{b})₂, —NHC(O)R^{a}—OH, —SR^{b}, or —R^{c}, wherein when the heteroaryl is a 5-membered heteroaryl with three nitrogen, it is substituted with —SR^{b};
each occurrence of R^{m} is each independently selected from the group consisting of -(L)ₜ-(R^{a})ₜ-(L)ₜ-(R^{a})ᵥ—R^{f}, -(L)ₜ-(R^{a}—O)_{q}—R^{a}—R^{f} and —Y—Rⁱ; or two of R^{m} collectively forms an oxo;
each occurrence of R^{v} is independently a 4-8 membered heterocycle or a 5-6 membered heteroaryl, each optionally substituted with one or more groups selected from C₁₋₃ alkyl, halo, oxo, acyl, —R^{a}—OR^{b}, —NR^{b}₂, —OR^{b}, —C(O)—R^{b}, —C(O)—OR^{b}, —C(O)—R^{d}, C₁₋₃ cycloalkyl, and —SR^{b};
each occurrence of n is independently 1, 2 or 3;
each occurrence oft is independently 0 or 1;
each occurrence of q is independently 1, 2, or 3; and
each occurrence of v is independently 0, 1, or 2,
   or a pharmaceutically acceptable salt thereof.
   Embodiment 31-3. A compound having formula:

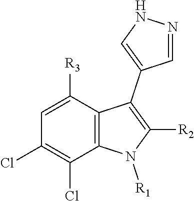

wherein:
R² is H, —C₁₋₃ alkyl, —CR⁵R⁷R⁸, —C(O)—R^{d}, —CH═N—R^{v}, or R^{w};
R¹ is H, —C₁₋₃ alkyl, —R^{a}—C(O)OH, —R^{a}—NH—C(O)CH₃, —R^{a}—NHS(O)₂CH₃, or 5- or 6-membered heteroaryl containing 1, 2, or 3 nitrogen atoms optionally substituted with one or more R^{m};
R⁵ is —R^{a}—OH, —OR^{b}, —N(R^{b})—C(O)—R^{a}—H, —N(R^{b})—C(O)—(R^{a})ₜ—N(R^{b})₂, —N(R^{b})—C(O)—(R^{a})ₜ—N(R^{b})—Rⁱ, —N(R^{b})—C(O)—(R^{a})ₜ—N(R^{b})—(R^{a})ₜ—C(O)—R^{b}, —N(R^{b})—C(O)—R^{d}, —N(R^{b})—R^{a}—R^{d}, —N(R^{b})—(C(O))ₜ—(R^{a})ₜ—Rⁱ, —N(R^{b})—S(O)₂—R^{a}—H, —C(O)—N(R^{b})—R^{a}—Rⁱ, —N(R^{b})—C(S)—R^{a}—H, or —R^{a}—C(O)OH;
—R³ is H, —YR^{b}, —YR^{c}, —YR^{d}—(NR^{b})ₙ—R^{a}—H, —Y—R^{a}—CN, —Y—C(O)R^{a}—H, —Y—R^{a}—C(O)N(R^{b})₂, —Y—C(S)R^{a}—H, —Y—C(O)R^{a}—F, —Y—C(O)—R^{a}—CN, or —Y—R^{a}—CH═CH—R^{a}—OH, wherein when R³ is H, then R² is other than H, —C₁₋₃ alkyl, —OH, or —C₁₋₃ alkoxy;
R⁷ and R⁸ are each R^{b}, or R⁷ and R⁸ collectively forms an oxo;
each occurrence of R^{a} is independently —C₁₋₃ alkylene- optionally substituted with one or more substituents selected from the group consisting of halo, —OH, —C₁₋₃ alkyl, —C₁₋₃ alkylene-OH, and —C₁₋₃ alkoxy;
each occurrence of R^{b} is independently —R^{a}—H or —H;
R^{c} is —C₃₋₆ cycloalkyl optionally substituted with one or more —OH, —R^{a}—H, or halo;
R^{d} is 4-8 membered heterocyclyl optionally substituted with oxo, —OH, —C₁₋₃ alkylcarbonyl, or —COOH;
each occurrence of L is selected from the group consisting of —C(O)—, —C(O)—N(R^{b})—, —C(O)—O—, —N(R^{b})—C(O)—, —O—C(O)—, —S(O)₂—, —N(R^{b})—S(O)₂—, and —N(R^{b})C(S)—;
each occurrence of Y is —O— or —N(R^{b})—;
each occurrence of R^{f} is selected from the group consisting of —R^{b}, —Rⁱ, —OR^{b}, —N(R^{b})₂, —(Y)ₜ—(R^{a})ₜ—R^{v}, and -L-R^{b};
each occurrence of Rⁱ is acyl or a 5- to 6-membered heteroaryl containing 1, 2, or 3 heteroatoms independently selected from nitrogen, oxygen, and sulfur and optionally substituted with —C₁₋₃ alkyl;
each occurrence of R^{w} is a 5- or 6-membered heteroaryl, wherein the heteroaryl includes three nitrogen or a combination of two nitrogen with a chalcogen, and wherein the heteroaryl is optionally substituted with —R^{b}, —R^{a}—OH, —CF₃, —N(R^{b})₂, —NHC(O)R^{a}—OH, —SR^{b}, or —R^{c}, wherein when the heteroaryl is a 5-membered heteroaryl with three nitrogen, it is substituted with —SR^{b};
each occurrence of R^{m} is each independently selected from the group consisting of -(L)ₜ-(R^{a})ₜ-(L)ₜ-(R^{a})ᵥ—R^{f}, -(L)ₜ-(R^{a}—O)_{q}—R^{a}—R^{f} and —Y—Rⁱ; or two of R^{m} collectively forms an oxo;
each occurrence of R^{v} is independently a 4-8 membered heterocycle or a 5-6 membered heteroaryl, each optionally substituted with one or more groups selected from C₁₋₃ alkyl, halo, oxo, acyl, —R^{a}—OR^{b}, —NR^{b}₂, —OR^{b}, —C(O)—R^{b}, —C(O)—OR^{b}, —C(O)—R^{d}, C₁₋₃ cycloalkyl, and —SR^{b};
each occurrence of n is independently 1, 2 or 3;
each occurrence oft is independently 0 or 1;
each occurrence of q is independently 1, 2, or 3; and
each occurrence of v is independently 0, 1, or 2,
   or a pharmaceutically acceptable salt thereof.
   Embodiment 31-4. A compound having formula:

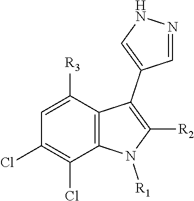

wherein:
R² is —CH₂R⁵;
R¹ and R⁵, together with the atoms to which they are immediately attached, form a 5-, 6-, or 7-membered heterocyclic ring containing 1 or 2 nitrogen atoms, wherein the heterocyclic ring is optionally substituted with one or more R^{m};
R³ is H, —YR^{b}, —YR^{c}, —YR^{d}, —(NR^{b})ₙ—R^{a}—H, —Y—R^{a}—CN, —Y—C(O)R^{a}—H, —Y—R^{a}—C(O)N(R^{b})₂, —Y—C(S)R^{a}—H, —Y—C(O)R^{a}—F, —Y—C(O)—R^{a}—CN, or —Y—R^{a}—CH═CH—R^{a}—OH, wherein when R³ is H, then R² is other than H, —C₁₋₃ alkyl, —OH, or —C₁₋₃ alkoxy;
each occurrence of R^{a} is independently —C₁₋₃ alkylene- optionally substituted with one or more substituents selected from the group consisting of halo, —OH, —C₁₋₃ alkyl, —C₁₋₃ alkylene-OH, and —C₁₋₃ alkoxy;
each occurrence of R^{b} is independently —R^{a}—H or —H;
R^{c} is —C₃₋₆ cycloalkyl optionally substituted with one or more —OH, —R^{a}—H, or halo;
R^{d} is 4-8 membered heterocyclyl optionally substituted with oxo, —OH, —C₁₋₃ alkylcarbonyl, or —COOH;
each occurrence of L is selected from the group consisting of —C(O)—, —C(O)—N(R^{b})—, —C(O)—O—, —N(R^{b})—C(O)—, —O—C(O)—, —S(O)₂—, —N(R^{b})—S(O)₂—, and —N(R^{b})C(S)—;
each occurrence of Y is —O— or —N(R^{b})—;
each occurrence of R^{f} is selected from the group consisting of —R^{b}, —Rⁱ, —OR^{b}, —N(R^{b})₂, —(Y)ₜ—(R^{a})ₜ—R^{v}, and -L-R^{b};
each occurrence of Rⁱ is acyl or a 5- to 6-membered heteroaryl containing 1, 2, or 3 heteroatoms independently selected from nitrogen, oxygen, and sulfur and optionally substituted with —C₁₋₃ alkyl;
each occurrence of R^{m} is each independently selected from the group consisting of -(L)ₜ-(R^{a})ₜ-(L)ₜ-(R^{a})ᵥ—R^{f}, -(L)ₜ-(R^{a}—O)_{q}—R^{a}—R^{f}, and —Y—Rⁱ; or two of R^{m} collectively forms an oxo;
each occurrence of R^{v} is independently a 4-8 membered heterocycle or a 5-6 membered heteroaryl, each optionally substituted with one or more groups selected from C₁₋₃ alkyl, halo, oxo, acyl, —R^{a}—OR^{b}, —NR^{b}₂, —OR^{b}, —C(O)—R^{b}, —C(O)—OR^{b}, —C(O)—R^{d}, C₁₋₃ cycloalkyl, and —SR^{b};
each occurrence of n is independently 1, 2 or 3;
each occurrence oft is independently 0 or 1;
each occurrence of q is independently 1, 2, or 3; and
each occurrence of v is independently 0, 1, or 2,
   or a pharmaceutically acceptable salt thereof.
   Embodiment 33. The compound of embodiment 1, wherein:
R⁴ is Cl;
X is Cl;
R⁶ is H;
W is CR²;
R¹ is selected from the group consisting of: H, —C₁₋₃ alkyl, —R^{a}—C(O)OH, —R^{a}—NH—C(O)CH₃, —R^{a}—NHS(O)₂CH₃,

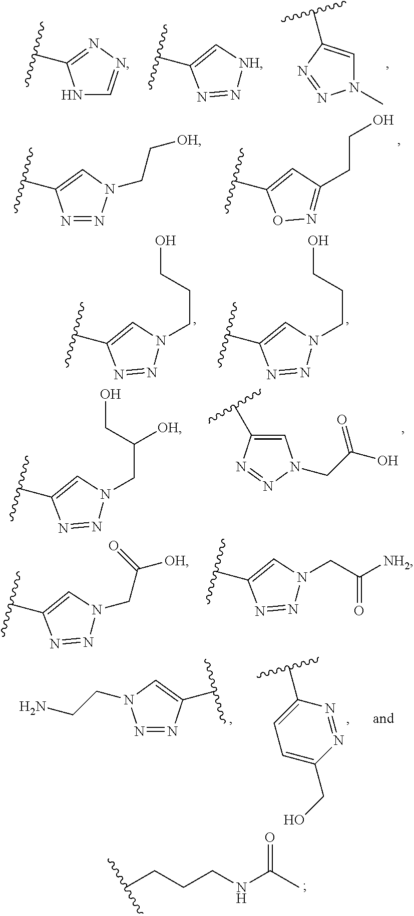

R² is selected from the group consisting of H, C₁₋₃ alkyl, —C₁₋₃ alkylene-OH, C₁₋₃ alkoxy,

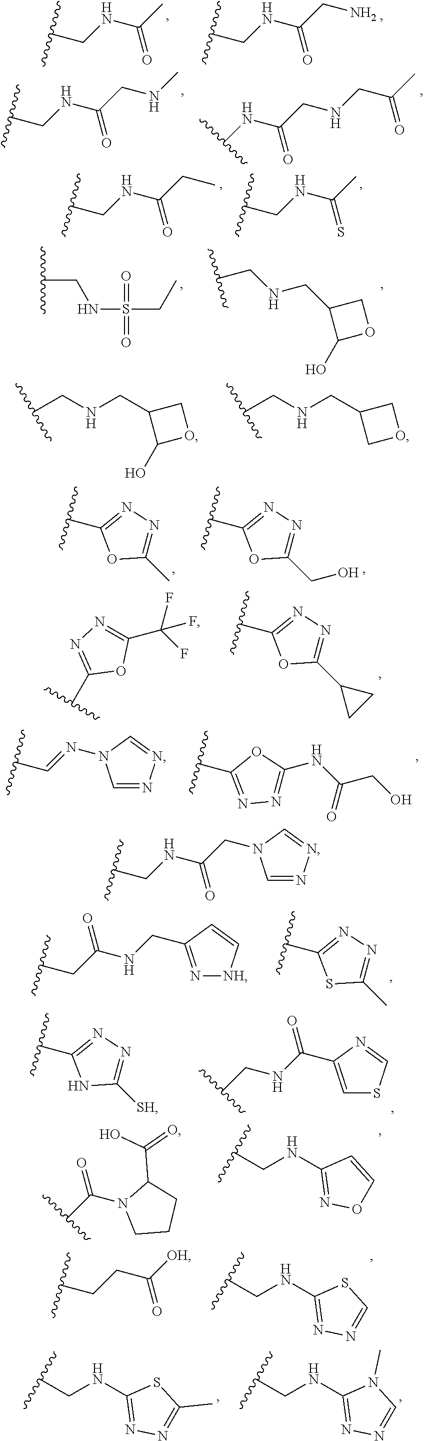

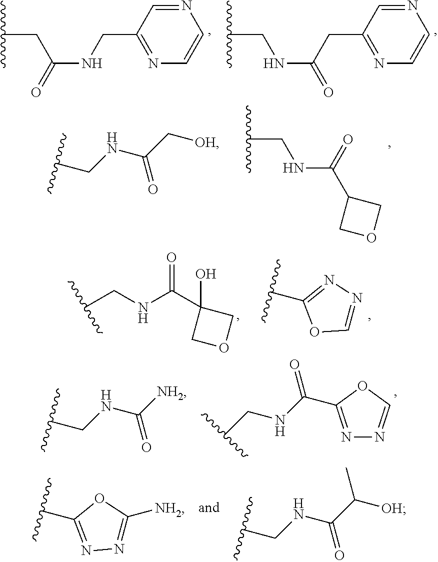

and
R³ is selected from the group consisting of H, C₁₋₃ alkoxy,

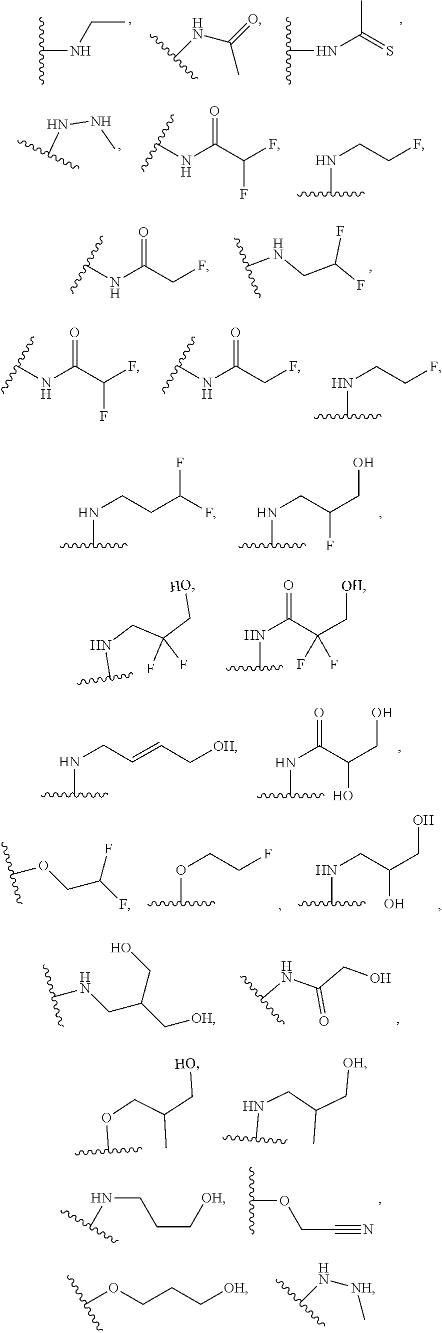

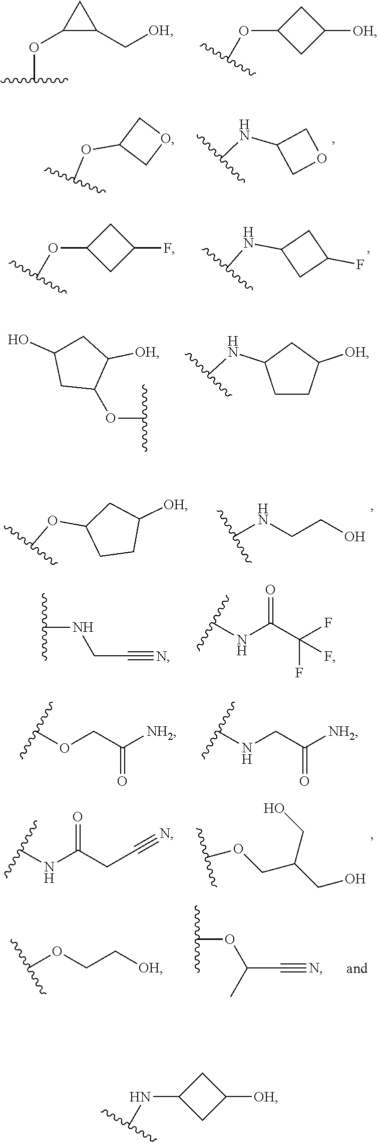

or a pharmaceutically acceptable salt thereof.
   Embodiment 33-1. A compound of formula:

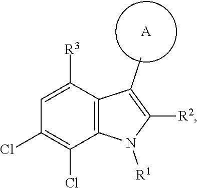

wherein
R¹ is selected from the group consisting of: H, —C₁₋₃ alkyl, —R^{a}—C(O)OH, —R^{a}—NH—C(O)CH₃, —R^{a}—NHS(O)₂CH₃,

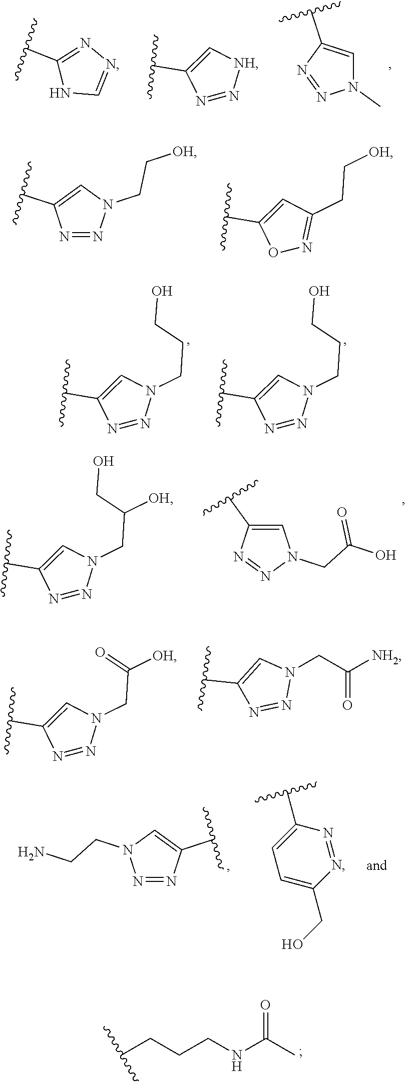

R² is selected from the group consisting of H, C₁₋₃ alkyl, —C₁₋₃ alkylene-OH, C₁₋₃ alkoxy,

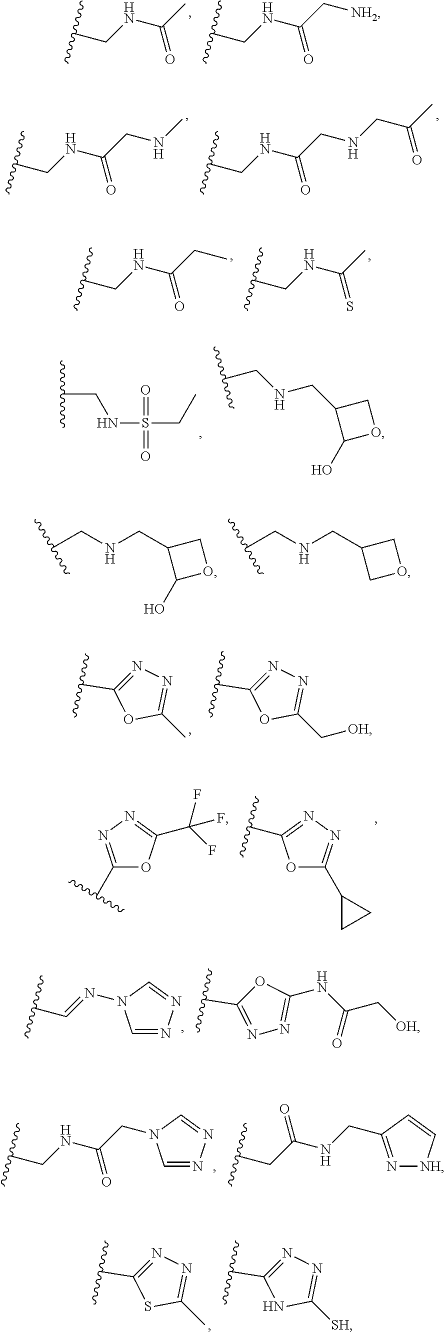

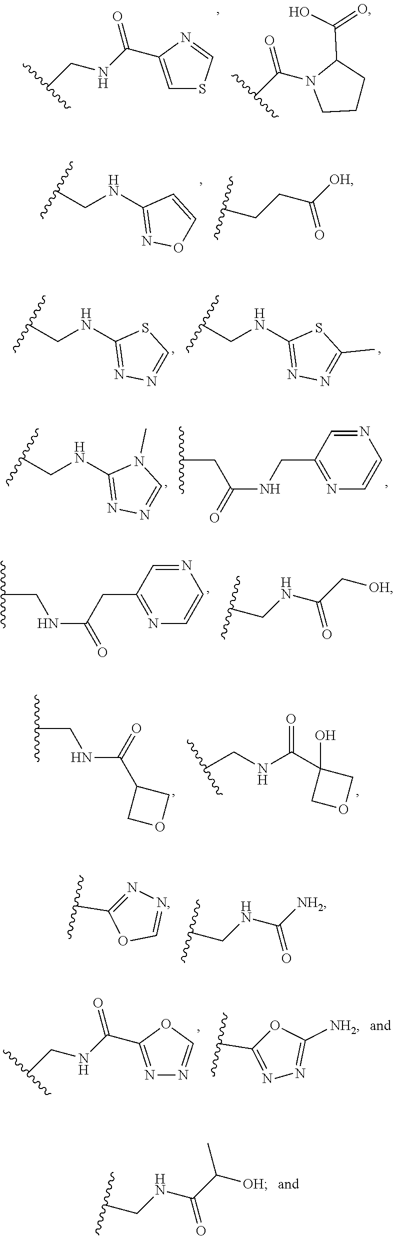

R³ is selected from the group consisting of H, C₁₋₃ alkoxy,

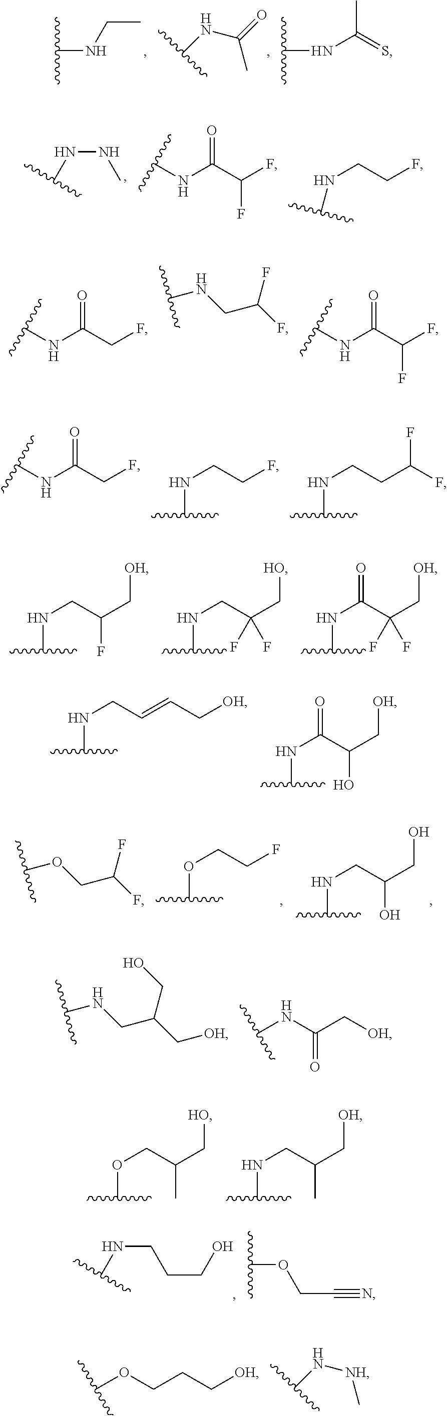

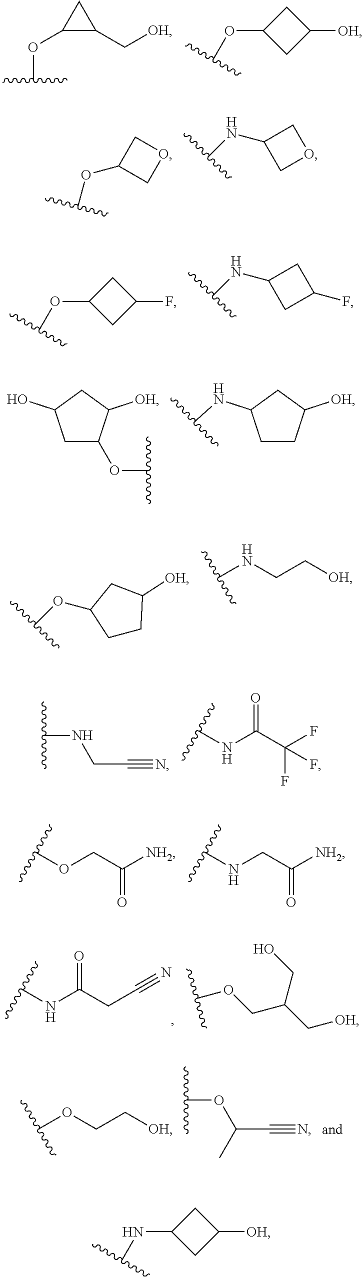

or a pharmaceutically acceptable salt thereof.
Embodiment 33-2. A compound of formula:

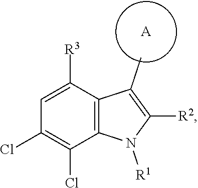

wherein:
R¹ is H or C₁₋₃ alkyl;
R² is selected from the group consisting of H, C₁₋₃ alkyl, —C₁₋₃ alkylene-OH, C₁₋₃ alkoxy,

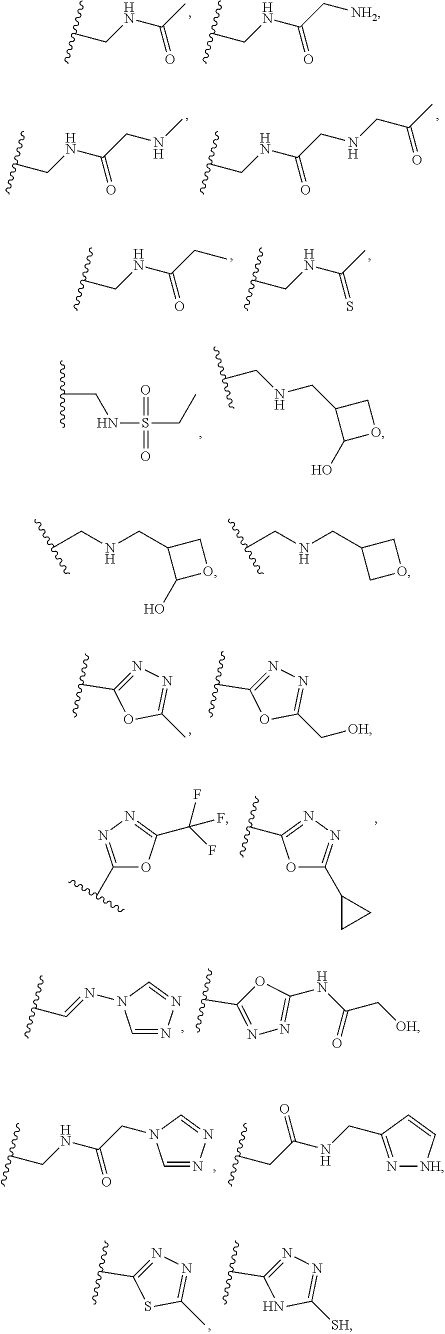

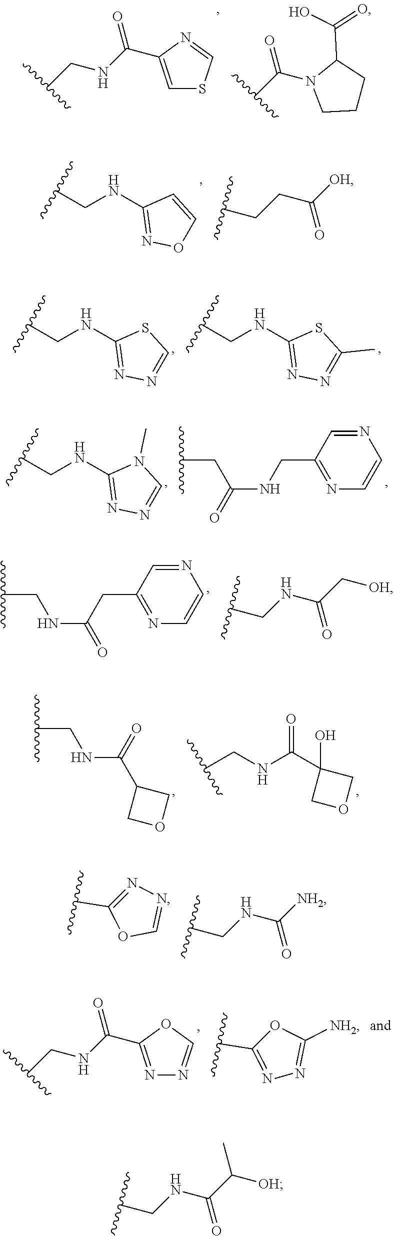

and
R³ is selected from the group consisting of H, C₁₋₃ alkoxy,

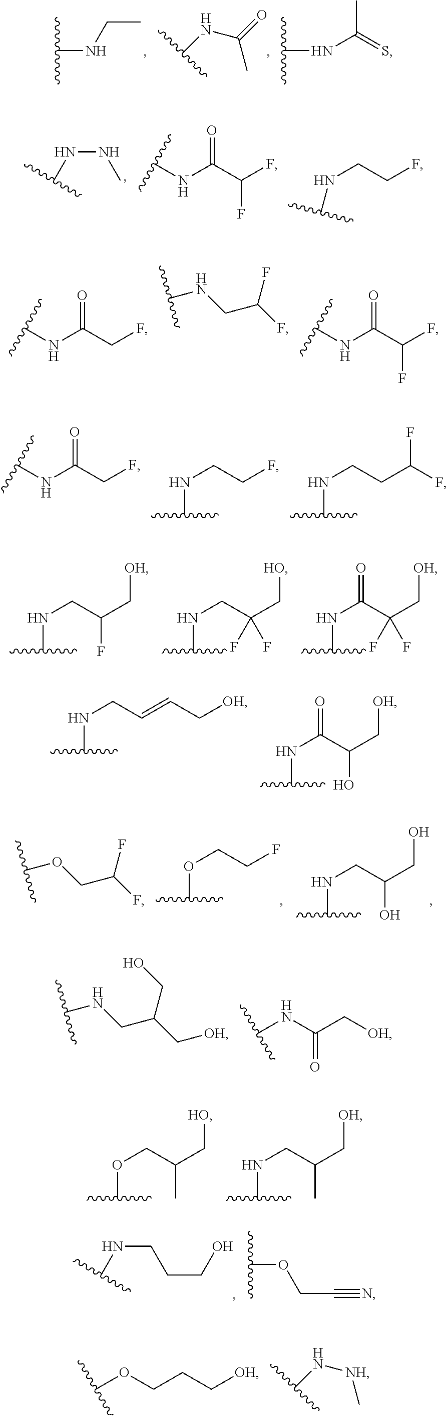

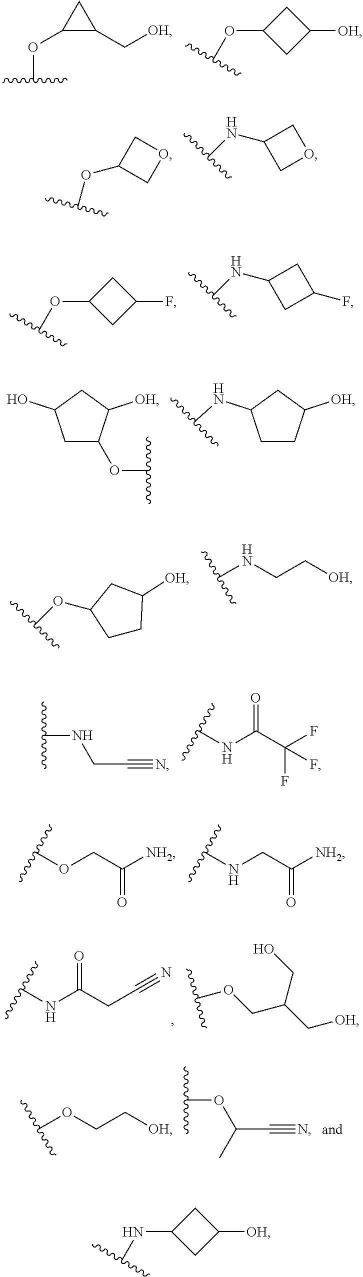

or a pharmaceutically acceptable salt thereof.
Embodiment 34. A compound having formula:

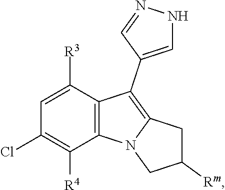

wherein:
R³ is

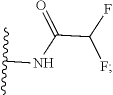 [SMILES: *NC(=O)C(F)F]

R⁴ is H or halo; and
R^{m} is selected from the group consisting of: —C₁₋₃ alkylene-OH,

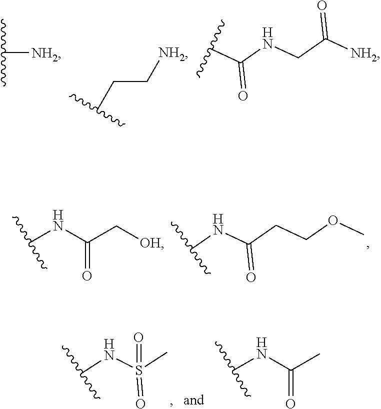

or a pharmaceutically acceptable salt thereof.
Embodiment 35. A compound having formula:

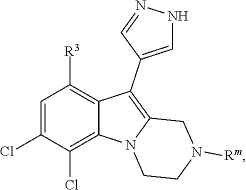

wherein:
R³ is selected from the group consisting of:

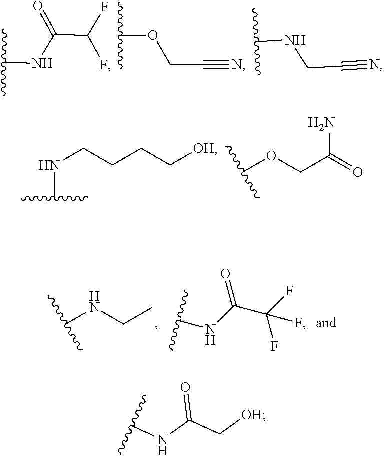

and
R^{m} is selected from the group consisting of: H,

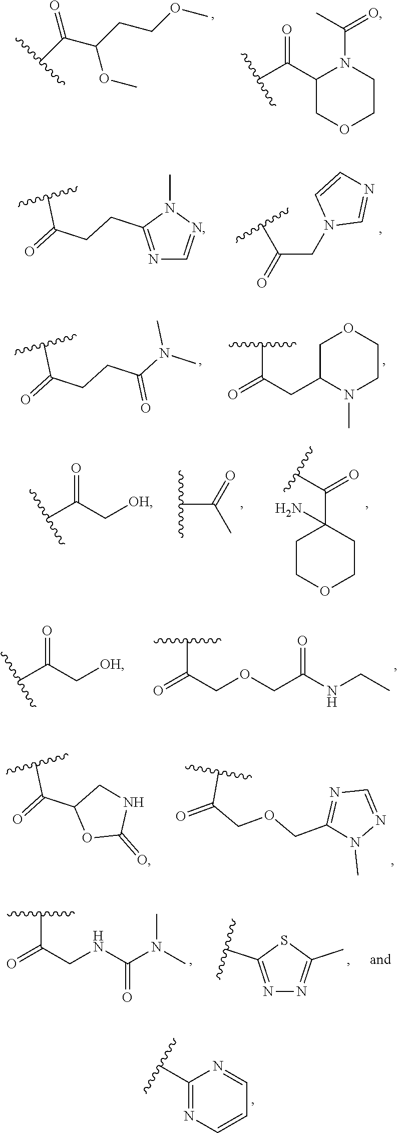

or a pharmaceutically acceptable salt thereof.
Embodiment 36. The compound of embodiment 1, 2, 3, 4, 5, 6, 7, 8, 9, 10, 11, 12, 13, 16, 17, 18, 19, 20, 21, 22, 23, 24, 25, 26, 29, or 30, wherein R^{m} is —C(O)—CH₂—O—CH₃, or a pharmaceutically acceptable salt thereof.
Embodiment 37. The compound of embodiment 1, having a formula of:

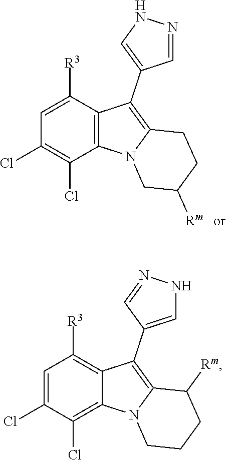

wherein:
R³ is selected from the group consisting of: H, C₁₋₃ alkoxy,

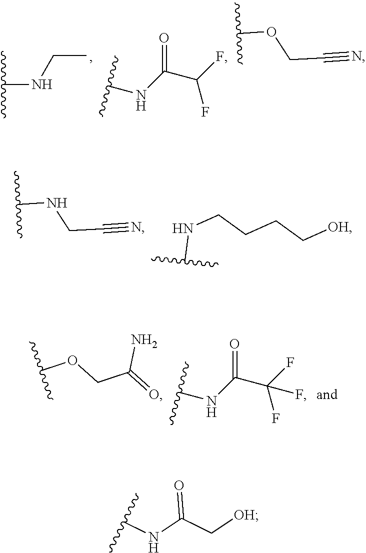

and
R^{m} is selected from the group consisting of H, —C₁₋₃ alkylene-OH,

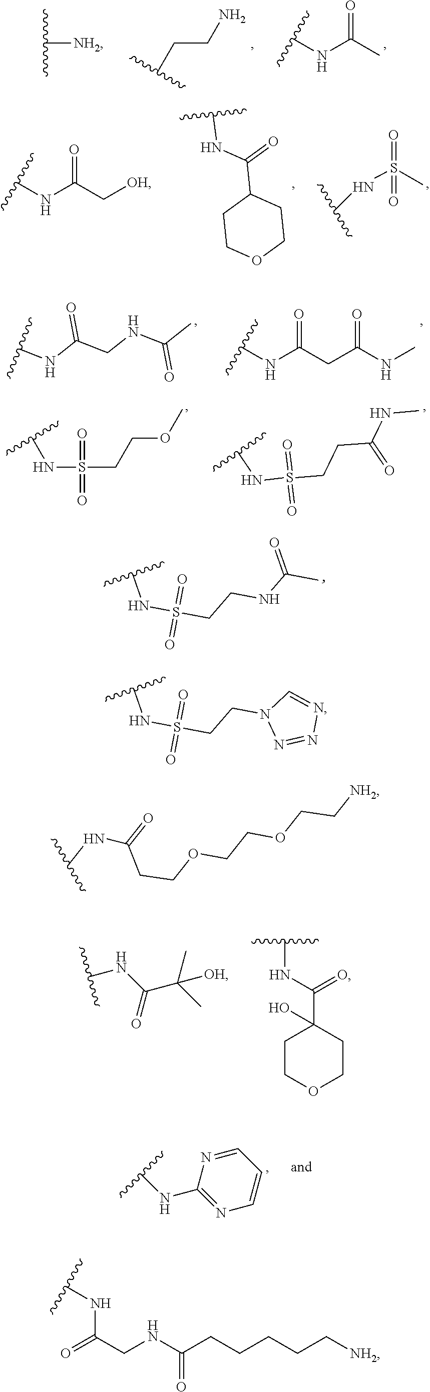

or a pharmaceutically acceptable salt thereof.
Embodiment 38. The compound of embodiment 1, having a formula of:

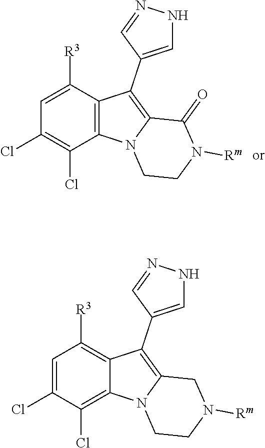

wherein:
R³ is selected from the group consisting of:

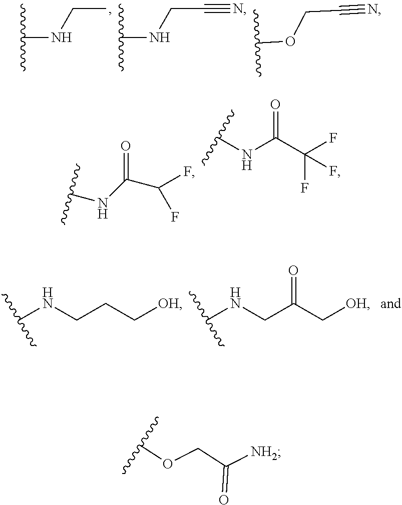

and
R^{m} is selected from the group consisting of H, —C₁₋₃ alkylene-OH, —C₁₋₃ alkoxy,

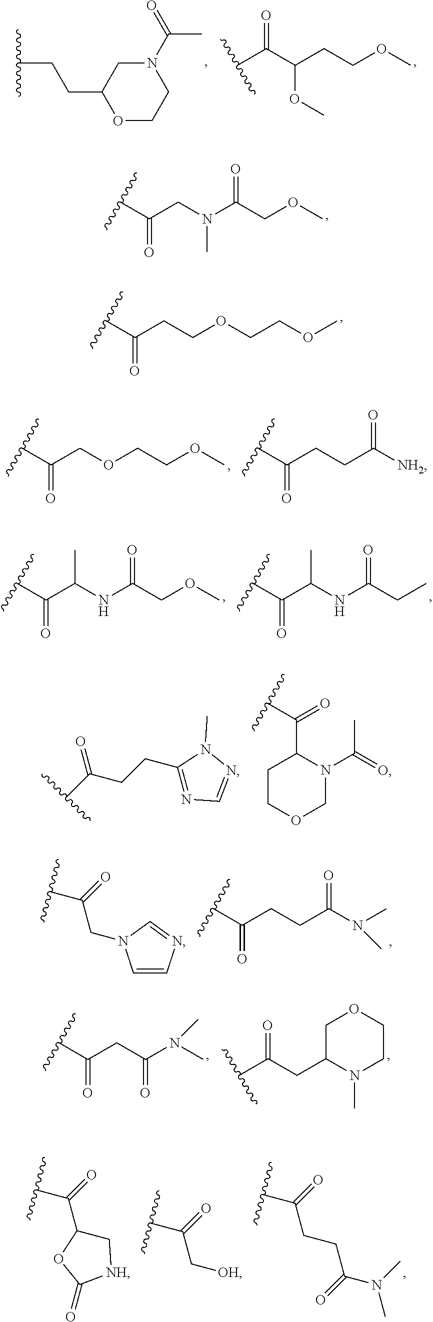

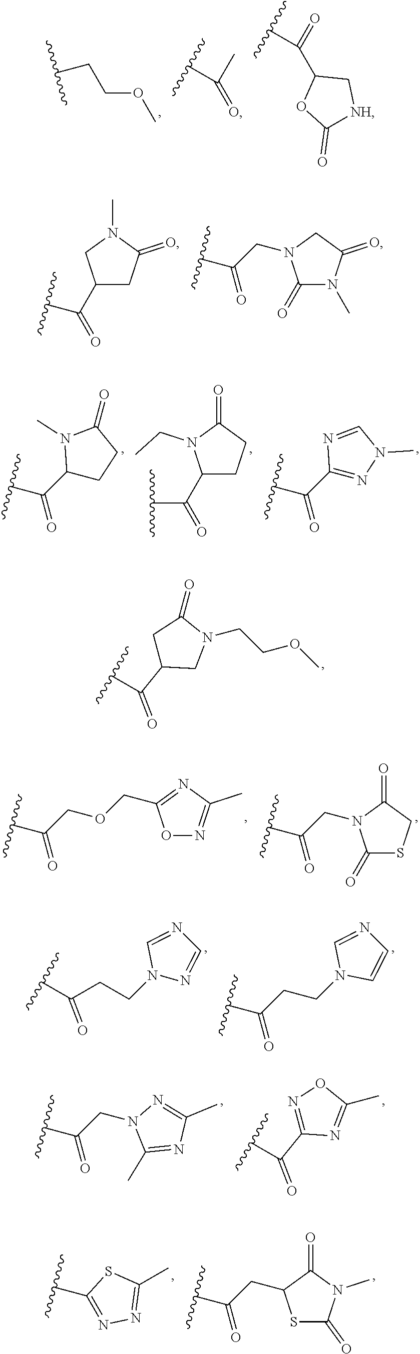

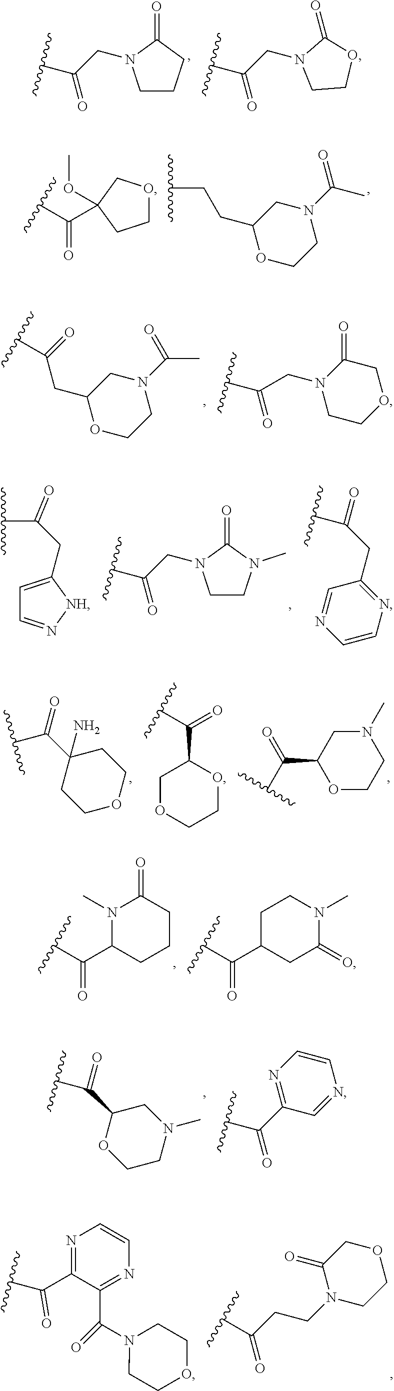

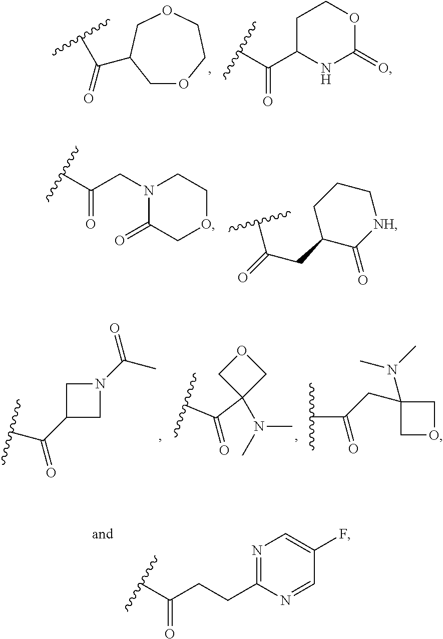

or a pharmaceutically acceptable salt thereof.
Embodiment 40. The compound of embodiment 1 or 2, wherein the ring A is

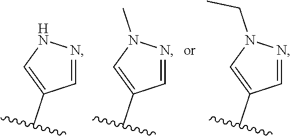

each optionally substituted with 1 or 2 C₁₋₃ alkyl, or a pharmaceutically acceptable salt thereof.
Embodiment 40-1. The compound of embodiment 1 or 2, wherein the ring A is

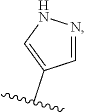 [SMILES: *c1cn[nH]c1]

or a pharmaceutically acceptable salt thereof.
Embodiment 40A. The compound of embodiment 1 or 2, wherein the ring A is optionally substituted

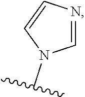 [SMILES: *n1ccnc1]

or a pharmaceutically acceptable salt thereof.
Embodiment 40B. The compound of embodiment 1 or 2, wherein the ring A is optionally substituted

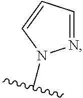 [SMILES: *n1cccn1]

or a pharmaceutically acceptable salt thereof.
Embodiment 41. The compound of embodiment 1, 2, 3, 4, 5, 6, 7, 8, 9, 10, 11, 12, 13, 16, 17, 18, or 19, wherein X is bromo, and R₄ is chloro, or a pharmaceutically acceptable salt thereof.
Embodiment 42. The compound of embodiment 1, 2, 3, 4, 5, 6, 7, 8, 9, 10, 11, 12, 13, 16, 17, 18, or 19, wherein X is chloro, and R₄ is chloro, or a pharmaceutically acceptable salt thereof.
Embodiment 42A. The compound of embodiment 1, 2, 3, 15, 20, 23, 27, 28, or 29, wherein R² is —CH₂NHRⁱ and R³ is —O—R^{a}—CN, or a pharmaceutically acceptable salt thereof.
Embodiment 42A-1. The compound of embodiment 1, 2, 3, 15, 20, 23, 27, 28, or 29, wherein R³ is —O—CH₂—CN, and R² is —CH₂NHRⁱ.
Embodiment 42A-2. The compound of embodiment 1, 2, 3, 15, 20, 23, 27, 28, or 29, wherein R² is H and R³ is —NH—C(O)—R^{a}—R^{b}, or a pharmaceutically acceptable salt thereof.
Embodiment 42A-3. The compound of embodiment 1, 2, 3, 15, 20, 23, 27, 28, or 29, wherein R³ is —NHC(O)CH₃ and R₂ is H, or a pharmaceutically acceptable salt thereof.
Embodiment 43. The compound according to embodiment 1, wherein the compound is selected from Table 3 and 6 (see below) or a pharmaceutically acceptable salt thereof.
Embodiment 43-1. A compound having formula:

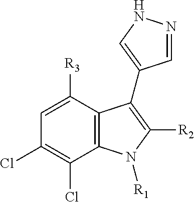

wherein:
R¹ is H or —C₁₋₃ alkyl;
R² is H, —C₁₋₃ alkyl, —CH₂R⁵;
R⁵ is —OR^{b}, —N(R^{b})—C(O)—R^{a}—H wherein R^{a} is —C₁₋₃ alkylene- optionally substituted with hydroxy, and R^{b} is R^{a}—H or H;
or
R¹ and R⁵, together with the atoms to which they are immediately attached, form a 5-, 6-, or 7-membered heterocyclic ring containing 1 or 2 nitrogen atoms optionally substituted with —C(O)—R^{a}—OR^{b}; and
R³ is selected from the group consisting of:
   H,
   —NH—C(O)—R^{a}—H wherein R^{a} is —C₁₋₃ alkylene- optionally substituted with one or two —OH, one or two F, or combinations thereof,
   —NH—R^{a}—H wherein R^{a} is —C₁₋₃ alkylene- optionally substituted with one or two —OH, one or two F, or combinations thereof,
   —O—R^{a}—H wherein R^{a} is —C₁₋₃ alkylene- optionally substituted with one or two —OH, one or two F, or combinations thereof, and
   O—CH₂—CN,
      or a pharmaceutically acceptable salt thereof.
      Embodiment 43-2. A compound having formula:

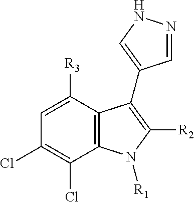

wherein:
R¹ is H or —CH₃;
R² is H, —CH₃, —CH₂OH, —CH₂—NHC(O)—CH₃, or —CH₂—NHC(O)—CH₂OH; or
R¹ and R², together with the atoms to which they are immediately attached, form

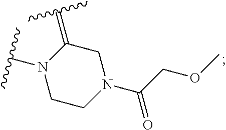 [SMILES: *N1CCN(C(=O)COC)CC1=*]

and
R³ is selected from the group consisting of H, —NH—C(O)—CH₃, —NH—C(O)—CF₂H, —NH—(CH₂)₃—OH, —O—(CH₂)₃—OH, and —O—CH₂—CN,
   or a pharmaceutically acceptable salt thereof.
   Embodiment 43-3. A compound, selected from the group consisting of:

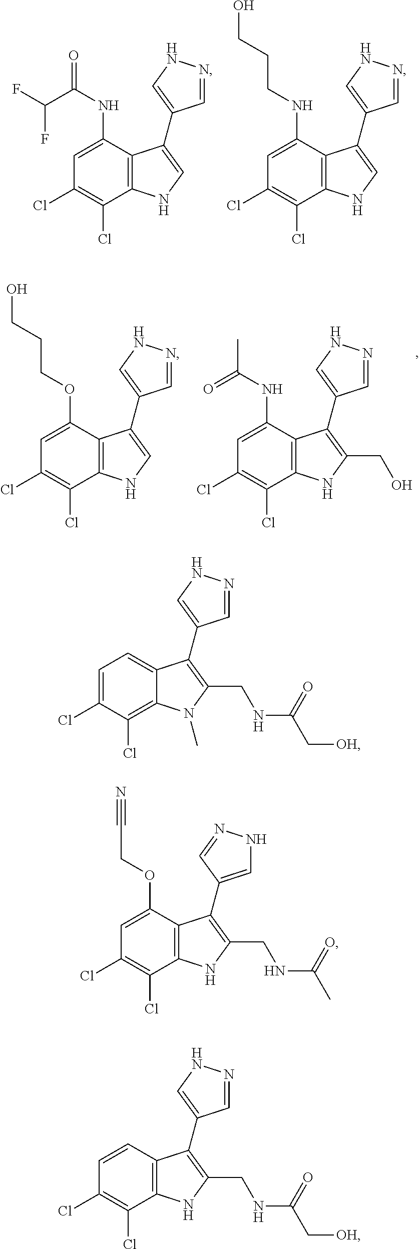

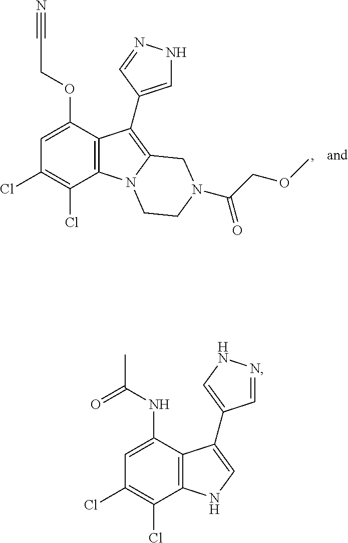 [SMILES: COCC(=O)N1CCn2c(c(-c3cn[nH]c3)c3c(OCC#N)cc(Cl)c(Cl)c32)C1 ; CC(=O)Nc1cc(Cl)c(Cl)c2[nH]cc(-c3cn[nH]c3)c12]

or a pharmaceutically acceptable salt thereof.
Embodiment 44. A pharmaceutical composition, comprising a compound according to any one of embodiments 1 to 43, or a pharmaceutically acceptable salt thereof, with one or more pharmaceutically acceptable carriers, diluents, or excipients.
Embodiment 44-1. A pharmaceutical composition, for use in the treatment of an immune-mediated disease in a patient, comprising a compound according to any one of embodiments 1 to 43, or a pharmaceutically acceptable salt thereof.
Embodiment 44-2. A pharmaceutical unit dosage composition, for use in the treatment of an immune-mediated disease in a patient, comprising a compound according to any one of embodiments 1 to 43, or a pharmaceutically acceptable salt thereof.
Embodiment 45. A method of treating an immune-mediated disease in a patient, comprising administering to a patient in need of such treatment an effective amount of a compound or salt according to any one of embodiments 1 to 43, or a pharmaceutically composition according to embodiment 44.
Embodiment 45-1. A method of treating an immune-mediated disease in a patient, comprising administering to a patient in need of such treatment an effective amount of a compound or salt according to any one of embodiments 1 to 43, a pharmaceutically composition according to embodiment 44-1, or a pharmaceutical unit dosage composition according to embodiment 44-2.
Embodiment 46. A method of treating systemic lupus erythematosus in a patient, comprising administering to a patient in need of such treatment an effective amount of a compound or salt according to any one of embodiments 1 to 43, or a pharmaceutically composition according to embodiment 44.
Embodiment 46-1. A method of treating systemic lupus erythematosus in a patient, comprising administering to a patient in need of such treatment an effective amount of a compound or salt according to any one of embodiments 1 to 43, a pharmaceutically composition according to embodiment 44-1, or a pharmaceutical unit dosage composition according to embodiment 44-2.
Embodiment 47. A method of treating lupus nephritis in a patient, comprising administering to a patient in need of such treatment an effective amount of a compound or salt according to any one of embodiments 1 to 43, or a pharmaceutically composition according to embodiment 44.
Embodiment 47-1. A method of treating lupus nephritis in a patient, comprising administering to a patient in need of such treatment an effective amount of a compound or salt according to any one of embodiments 1 to 43, a pharmaceutically composition according to embodiment 44-1, or a pharmaceutical unit dosage composition according to embodiment 44-2.
Embodiment 48. A method of treating dermatomyositis in a patient, comprising administering to a patient in need of such treatment an effective amount of a compound or salt according to any one of embodiments 1 to 43, or a pharmaceutically composition according to embodiment 44.
Embodiment 48-1. A method of treating dermatomyositis in a patient, comprising administering to a patient in need of such treatment an effective amount of a compound or salt according to any one of embodiments 1 to 43, a pharmaceutically composition according to embodiment 44-1, or a pharmaceutical unit dosage composition according to embodiment 44-2.
Embodiment 49. A method of treating Aicardi-Goutières syndrome in a patient, comprising administering to a patient in need of such treatment an effective amount of a compound or salt according to any one of embodiments 1 to 43, or a pharmaceutically composition according to embodiment 44.
Embodiment 49-1. A method of treating Aicardi-Goutières syndrome in a patient, comprising administering to a patient in need of such treatment an effective amount of a compound or salt according to any one of embodiments 1 to 43, a pharmaceutically composition according to embodiment 44-1, or a pharmaceutical unit dosage composition according to embodiment 44-2.
Embodiment 50. A method of treating a disease associated with cGAS activation in a patient, comprising administering to a patient in need of such treatment an effective amount of a compound or salt according to any one of embodiments 1 to 43, or a pharmaceutically composition according to embodiment 44.
Embodiment 50-1. A method of treating a disease associated with cGAS activation in a patient, comprising administering to a patient in need of such treatment an effective amount of a compound or salt according to any one of embodiments 1 to 43, a pharmaceutically composition according to embodiment 44-1, or a pharmaceutical unit dosage composition according to embodiment 44-2.
Embodiment 51. A compound, or a pharmaceutically acceptable salt thereof, according to any one of embodiments 1 to 43 for use in therapy.
Embodiment 52. A compound, or a pharmaceutically acceptable salt thereof, according to any one of embodiments 1 to 43 for use in the treatment of an immune-mediated disease.
Embodiment 53. A compound, or a pharmaceutically acceptable salt thereof, according to any one of embodiments 1 to 43 for use in the treatment of systemic lupus erythematosus.
Embodiment 54. A compound, or a pharmaceutically acceptable salt thereof, according to any one of embodiments 1 to 43 for use in the treatment of lupus nephritis.
Embodiment 55. A compound, or a pharmaceutically acceptable salt thereof, according to any one of embodiments 1 to 43 for use in the treatment of dermatomyositis.
Embodiment 56. A compound, or a pharmaceutically acceptable salt thereof, according to any one of embodiments 1 to 43 for use in the treatment of Aicardi-Goutières syndrome.
Embodiment 57. Use of a compound, or a pharmaceutically acceptable salt thereof, according to any one of embodiments 1 to 43, in the manufacture of a medicament for the treatment of an immune-mediated disease in a patient.

As used herein, the symbol “—” refers to an attachment point.

As used herein, a monoradical appearing in a parenthesis of a name of moiety (or substituent) designates a pendant branch attached to a segment of the moiety between two attachment points. The branch may be attached to the segment using a single covalent bond or a double bond. For example, —N(CH₃)—(CH₂)₂OH describes an amine diradical having two attachment points represented by “—” along with a pendant methyl branch. For another example, —C(O)— describes a carbon diradical having two attachment points represented by “-” with a pendant “oxo” branch. The term “oxo” refers to an oxygen atom as a substituent, and connected to the rest of the molecule with a double bond. The term “oxo” can also be denoted as “═O”. The oxo together with the carbon atom it attaches to forms a carbonyl group. In this instance, the pendant group attaches to the segment (i.e. the carbon atom) with a double bond.

As used herein, the term “alkyl”, used alone or as part of a larger moiety, refers to a saturated, straight, or branched chain hydrocarbon group containing one or more carbon atoms. Alkyl is generally monovalent. For example, “ethyl” can be represented as CH₃CH₂·, where the dot represents a radical or dangling bond. The term may be preceded with an indication of number of carbon atoms of the alkyl. Accordingly, the term “C₁₋₃ alkyl”, used alone or as part of a larger moiety, refers to a saturated, straight, or branched chain hydrocarbon group containing one, two, or three carbon atoms.

As used herein, the term “alkylene”, used alone or as part of a larger moiety, refers to a bivalent group that can be hypothetically constructed by removing from an alkyl group a terminal hydrogen atom that is remote from the attachment point of the alkyl group, thereby creating a second attachment point on the opposite end. For example, an “ethylene” can be represented as ·CH₂CH₂·, and can be regarded as derived from ethyl by removing a hydrogen atom from an end of the ethyl group. In other words, the term “alkylene” refers to a saturated, straight, or branched chain hydrocarbon group containing one or more carbon atoms as well as two attachment points on opposite ends (sometimes referred to as diradical). Accordingly, the term “C₁₋₃ alkylene” refers to a saturated, straight, or branched chain hydrocarbon group containing one, two, or three carbon atoms, as well as two attachment points on opposite ends. The term “alkylene” is typically used as part of a larger moiety, such as —C₁₋₃ alkylene-OH (referring to a modified C₁₋₃ alkyl with one terminal hydrogen atom replaced by a hydroxy), and —C₁₋₃ alkylene-NH₂ (referring to a modified C₁₋₃ alkyl with one terminal hydrogen atom replaced by an amino group).

As used herein, the term “alkylcarbonyl”, used alone or as part of a larger moiety, refers to a moiety having a carbonyl group directly attached to an alkyl group, where the attachment point of the moiety is on the carbonyl group. In other words, the term “alkylcarbonyl” refers to —C(O)-alkyl. The term may be preceded with an indication of number of carbon atoms of the alkyl. Accordingly, the term “—C₁₋₃ alkylcarbonyl” refers to —C(O)—C₁₋₃ alkyl.

As used herein, the term “alkoxy”, used alone or as part of a larger moiety, refers to a moiety having an oxygen directly attached to an alkyl group with the attachment point of the moiety on the oxygen atom. The term may be preceded with an indication of number of carbon atoms of the alkoxy. Accordingly, the term “C₁₋₃ alkoxy” refers to —O—C₁₋₃ alkyl. Representative alkoxyl groups include methoxy, ethoxy, propyloxy, tert-butoxy, and the like.

As used herein, the term “chalcogen” refers to an element of group 16 of the periodic table, such as oxygen (O) and sulfur (S).

As used herein, the term “cycloalkyl” refers to a saturated ring system containing at least three carbon atoms. The term may be preceded with an indication of number of carbon atoms of the cycloalkyl. Accordingly, the term “C₃₋₆ cycloalkyl” refers to a saturated ring system containing 3, 4, or 5 carbon atoms. Cycloalkyl can be monocyclic (having one ring), bicyclic (having two rings), or polycyclic (having two or more rings). Exemplary monocyclic cycloalkyl rings include cyclopropyl, cyclobutyl, cyclopentyl, cyclohexyl, and cycloheptyl.

As used herein, the term “halo” refers to halogen as a substituent, and specifically chloro, fluoro, bromo, or iodo.

As used herein, the term “heteroaryl”, unless specified, refers to groups having 5 to 10 ring atoms, preferably 5, 6, 9, or 10 ring atoms, having 6, 10, or 14 π-electrons shared in a cyclic array, and having heteroatoms. The term “heteroatom” refers to nitrogen, oxygen, or sulfur, and includes any oxidized form of nitrogen or sulfur, and any quaternized form of a basic nitrogen.

As used herein, the term “heterocyclic ring” refers to an optionally substituted saturated ring system containing at least two carbon atoms and at least one heteroatom. Exemplary heteroatoms are oxygen, nitrogen and sulfur. Exemplary heterocyclic rings include oxirane, aziridine, oxetane, oxolane, pyrrolidine, piperidine and morpholine. This term is used herein interchangeably with the terms “heterocyclyl” and “heterocycle”. These terms may be preceded with a designation of number of ring atoms. For example, “4-8 membered heterocyclyl” refers to such a saturated ring system having 4, 5, 6, 7, or 8 ring atoms, where the ring atoms include at least one heteroatom. Exemplary heteroatoms are oxygen, nitrogen, and sulfur. Exemplary heterocyclic rings (or heterocycles) include oxirane, aziridine, oxetane, oxolane, pyrrolidine, piperidine, and morpholine. Heterocycles can be monocycles (having one ring), bicycles (having two rings), or polycycles (having two or more rings) that may be, for example, fused with each other.

As used herein, the term “thiol” refers to “—SH” group, and the term “thioether” refers to an organosulfur moiety having a sulfur attached to an optionally substituted alkyl group, with the attachment point of the thioether on the sulfur atom.

As used herein, the term “stereoisomer” refers to an isomer made up of the same atoms bonded by the same bonds but having different and non-interchangeable structures in the three-dimensional space. The term of stereoisomer includes “enantiomer” which refers to two stereoisomers that are mirror images of one another and are not superimposable over one another. A one-to-one mixture of a pair of enantiomers is referred to as a “racemic” mixture. The term of stereoisomer also includes “diastereoisomers” (or “diastereomer”) which refers to two stereoisomers that have at least two asymmetric atoms, but which are not mirror-images of each other. The absolute stereochemistry of a stereoisomer may be specified according to the Cahn-Ingold Prelog R S system, where the stereochemistry at each chiral center is designated as either R or S. When stereoisomers are resolved yet whose absolute configuration is unknown, those stereocenters are designated (+) or (−) depending on the direction (dextro- or laevorotary) that they rotate the plane of polarization at the wavelength of the sodium D line. In structural illustrations, plain lines ( [symbol] ) depict bonds approximately in the plane of the drawing; bonds to atoms above the plane are shown with a bold wedge ( [symbol] ) starting from an atom in the plane of the drawing at the narrow end of the wedge; and bonds to atoms below the plane are shown with a hashed wedge of short parallel lines ( [symbol] ) starting from an atom in the plane of the drawing at the narrow end of the hashed wedge. For compounds having a stereocenter where all bonds connected to the stereocenter are structurally represented with plain lines ( [symbol] ), or the compounds are represented with chemical nomenclature without a stereoisomer designation, they shall be construed to mean any of the possible stereoisomers, a racemic mixture thereof, or a mixture with one or more stereoisomers enriched relative to others, unless explicitly stated otherwise. In other words, for any compound disclosed here that includes a stereocenter to which all bonds connected are illustrated in plain lines, the disclosure contemplates each and every possible stereoisomer thereof, as well as any mixture of those stereoisomers.

The term stereoisomer also includes “geometric isomers” such as “cis-trans isomer”. These are isomers where arrangements of groups around a double bond or a ring differ from one another despite the same molecular formula. The term of stereoisomer may further include “rotational isomers” or “rotamers” which is defined as stereoisomers that arise from hindered single-bond rotation. For simplicity, the term “stereoisomer” is used here interchangeable with the term “isomer”. Unless explicitly stated otherwise (such as by referencing the retention time for the specified separation condition), “isomer 1” refers to the stereoisomer that eludes out first from the column during separation (e.g. a chiral separation of a racemic mixture or a separation of a pair of cis-trans isomers) under a stated separation condition; and “isomer 2” refers to the stereoisomer that eludes out the second during the same separation. An asterisk (*) is used to denote the chiral center in the structures for “isomer 1” and “isomer 2”. Moreover, when the asterisk is used on a stereocenter to which a bold wedge ( [symbol] ) or a hashed wedge ( [symbol] ) is also attached, the solid wedge and hashed wedge represents relative stereochemistry only, and are not designations of absolute stereochemistry.

As used herein, the term “immune-mediated disease” encompasses a group of autoimmune or inflammatory disorders in which immunological pathways play an important etiological and/or pathogenetic role. Such diseases are sometimes characterized by an alteration in cellular homeostasis. Immune-mediated diseases may be triggered by environmental factors, dietary habits, infectious agents, and genetic predisposition. Immune-mediated disease includes, for example, systemic lupus erythematosus, lupus nephritis, dermatomyositis, and Aicardi-Goutières syndrome.

As used herein, the term “patient” refers to a human.

As used herein, the term “treating” includes restraining, slowing, stopping, or reversing the progression or severity of an existing symptom or disorder.

As used herein, the term “effective amount” refers to the amount or dose of compound of the invention, or a pharmaceutically acceptable salt thereof which, upon single or multiple dose administration to the patient, provides the desired effect in the patient under diagnosis or treatment. An effective amount can be readily determined by one skilled in the art by the use of known techniques. In determining the effective amount for a patient, a number of factors are considered, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances. The compounds of the present invention are generally effective over a wide dosage range. For example, dosages per day normally fall within the range of about 0.1 to about 15 mg/kg of body weight.

The compounds of the present invention are preferably formulated as pharmaceutical compositions administered by any route which makes the compound bioavailable, including oral and transdermal routes. Most preferably, such compositions are for oral administration. Such pharmaceutical compositions and processes for preparing same are well known in the art (See, e.g., Remington: The Science and Practice of Pharmacy, A. Adej are, Editor, 23rd Edition, Elsevier Academic Press, 2020).

The compounds of the present invention, or pharmaceutically acceptable salts thereof, may be prepared according to the following Preparations and Examples by methods well known and appreciated in the art. Suitable reaction conditions for the steps of these Preparations and Examples are well known in the art and appropriate substitutions of solvents and co-reagents are within the skill of the art. Likewise, it will be appreciated by those skilled in the art that synthetic intermediates may be isolated and/or purified by various well-known techniques as needed or desired, and that frequently, it will be possible to use various intermediates directly in subsequent synthetic steps with little or no purification. As an illustration, compounds of the preparations and examples can be isolated, for example, by silica gel purification, isolated directly by filtration, or crystallization. Furthermore, the skilled artisan will appreciate that in some circumstances, the order in which moieties are introduced is not critical. The particular order of steps required to produce the compounds of the present invention is dependent upon the particular compound being synthesized, the starting compound, and the relative liability of the substituted moieties, and is well appreciated by the skilled chemist. All substituents, unless otherwise indicated, are as previously defined, and all reagents are well known and appreciated in the art.

TABLE 1
Abbreviations and definitions

| Term | Definition |
|---|---|
| Ac₂O | Acetic anhydride |
| ACN | Acetonitrile |
| BOC/Boc | tert-Butyloxycarbonyl |
| Boc₂O | Di-tert-butyl dicarbonate |
| BFMO | N,N′-bis(furan-2-ylmethyl)oxamide |
| BrettPhos-Pd- | [(2-Di-cyclohexylphosphino-3,6-dimethoxy-2′,4′,6′- |
| G3 | triisopropyl-1,1′-biphenyl)-2-(2′-amino-1,1′-biphenyl)] |
| | palladium(II) methanesulfonate |
| tert- | [(2-Di-tert-butylphosphino-3,6-dimethoxy-2′,4′,6′- |
| BuBrettPhos- | triisopropyl-1,1′-biphenyl)-2-(2′-amino-1,1′-biphenyl)] |
| Pd-G3 | palladium(II) methanesulfonate |
| t-BuOK | Potassium tert-butoxide |
| t-BuONa | Sodium tert-butoxide |
| (t-Bu₃P)₂Pd | Bis(tri-tert-butylphosphine)palladium(0) |
| C18 | Octadecylsilane |
| DBU | 1,8-Diazabicyclo[5.4.0]undec-7-ene |
| DCM | Dichloromethane |
| DEA | Diethylamine |
| DIAD | Diisopropyl azodicarboxylate |
| DIBAL-H | Diisobutylaluminum hydride |
| DIPEA | N,N-Diisopropylethylamine |
| DMAP | 4-Dimethylaminopyridine |
| DME | Dimethoxyethane |
| DMEA | Dimethylethylamine |
| DMEDA | 1,2-Dimethylethylenediamine |
| DMF | N,N-Dimethylformamide |
| DMSO | Dimethyl sulfoxide |
| DPPA | Diphenylphosphoryl azide |
| dppf | 1,1′-Bis(diphenylphosphino)ferrocene |
| dtbpf | 1,1′-Bis(di-tert-butylphosphino)ferrocene |
| EDCI | 1-Ethyl-3-(3-dimethylaminopropyl)carbodiimide |
| ee | Enantiomeric excess |
| equiv | Equivalent(s) |
| Et₂O | Diethyl ether |
| ES/MS | Electrospray mass spectrometry |
| EtOAc | Ethyl acetate |
| EtOH | Ethanol |
| HATU | 1-[Bis(dimethylamino)methylene]-1H-1,2,3-triazolo |
| | [4,5-b]pyridinium 3-oxide hexafluorophosphate |
| HOBt | 1H-1,2,3-Benzotriazol-1-ol |
| HPLC | High performance liquid chromatography |
| Hr/hrs | Hour/hours |
| IPA | Isopropanol |
| IPAm | Isopropylamine |
| LAH | Lithium aluminium hydride |
| LC | Liquid chromatography |
| LCMS | Liquid chromatography mass spectrometry |
| LDA | Lithium diisopropylamide |
| MeOH | Methanol |
| min | Minute(s) |
| MS | Mass spectrometry |
| MsC1 | Methanesulfonyl chloride |
| MTBE | Methyl tert-butyl ether |
| MW | Molecular weight |
| NBS | N-Bromosuccinimide |
| NIS | N-Iodosuccinimide |
| NMP | N-Methylpyrrolidone |
| NMR | Nuclear magnetic resonance |
| PEG-400 | Polyethylene glycol 400 |
| Pd₂(dba)₃ | Tris(dibenzylideneacetone)dipalladium(0) |
| Pd(dtbpf)Cl₂ | [1,1′-Bis(di-tert-butylphosphino)ferrocene] |
| | dichloropalladium(II) |
| Pd(PhCN)₂Cl₂ | Bis(benzonitrile)palladium(II) chloride |
| Pet ether | Petroleum ether |
| Ph₃P | Triphenylphosphine |
| PPA | Polyphosphoric acid |
| ppm | Parts per million |
| RT | Room temperature |
| Rt | Retention time |
| SCX | Strong cation exchange |
| SEM | [2-(Trimethylsilyl)ethoxy]methyl |
| SFC | Supercritical fluid chromatography |
| TBAF | Tetrabutylammonium fluoride |
| TCDI | Thiocarbonyldiimidazole |
| TEA | Triethylamine |
| TFA | Trifluoroacetic acid |
| TFAA | Trifluoroacetic anhydride |
| THE | Tetrahydrofuran |
| THP | Tetrahydropyran |
| TLC | Thin-layer chromatography |
| TMSN₃ | Trimethylsilyl azide |
| TosMIC | Toluenesulfonylmethyl isocyanide |
| Ts | Tosyl |
| TsCl | 4-Toluenesulfonyl chloride |
| wt | Weight |
| Xantphos | (9,9-Dimethyl-9H-xanthene-4,5-diyl)bis |
| | (diphenylphosphane) |

The general syntheses for compounds of this disclosure are described in Schemes 1 to 3, where Q represents O or NH; X₂ represents bromo (Br) or iodo (I); Pg represents a suitable protecting group (such as SEM, Boc, Ts, and —SO₂Ph); R₁ represents Cl, F, CN, or H; R₂ represents an alkyl or amide substituent; and R₃ represents an amide, carbamate, urea, alkyl, or aryl substituent.

In schemes 1 to 3, Rxn 1 depicts protection of an indole nitrogen under conditions well known in the art using groups such as SEM, tosyl, benzene sulfonyl, or Boc to give compound 2A. A person of ordinary skill in the art will recognize typical protection employs a strong base such as NaH or LDA in a suitable solvent such as THF with the appropriate electrophilic protecting group reagent.

Rxn 2 depicts, where Q is NH, a Buchwald amination or amidation reaction employing a suitable catalyst, preferably a palladium catalyst, and a base, with an appropriate coupling partner, for example, primary amides or amines to give compounds such as 3A, 4A, 8, 15A, or 20A. Reactions may be run at elevated temperatures under an inert atmosphere.

Rxn 3 depicts the indole deprotection of the SEM, tosyl, benzene sulfonyl, or Boc protecting groups under conditions well known in the art to give compounds such as 4A, or 10A. A person of ordinary skill in the art will recognize typical deprotection conditions may include acidic (e.g., HCl, TFA), basic (e.g., NaOH) or with fluoride-based reagents (e.g., TBAF) in suitable solvents such as DCM, THF, and water.

Rxn 4 depicts halogenation of indole typically using NB S or NIS in DMF at room temperature to give compounds such as 4A, 5A, 11A, 13A, or 23A. The reaction is carried out in a suitable solvent such as DMF.

Rxn 5 depicts Suzuki cross-coupling of THP-protected pyrazole pinacol boronate to give compounds such as 6A, 12A, 14A, or 23B. The process may start with synthesis of pyrazolyl boronic ester. The boronic ester is then coupled with a variety of different heteroaryl halides under Suzuki cross-coupling type conditions well known in the art (such as use of palladium catalyst in dioxane). This allows for the reaction of substituted indole halides under mild conditions such as temperatures of between 90° C. and 100° C.

Rxn 6 depicts deprotection of pyrazole nitrogen by removing the THP group, and where appropriate, deprotection of the aniline nitrogen by removing the Boc protecting group, under acidic conditions (e.g., HCl, TFA) to give compounds such as 6B, 16A, or 24A.

Rxn 7 depicts a Pd-catalyzed cross-coupling reaction of tert-butyl carbamate with various heteroaryl halides to give compounds such as 7A, 17B, or 25A. The heteroaryl halide is contacted with a palladium catalyst such as XantPhos-Pd-G₂, a base such as Cs₂CO₃, and a solvent such as 1,4-dioxane in a suitable temperature (about 100° C.).

Rxn 8 depicts hydroxylation of aryl bromide. The hydroxylation process is promoted by a catalyst based on a biarylphosphine ligand tBuBrettPhos and its corresponding palladium precatalyst to give compounds such as 8A, 18A, or 26A. A person of ordinary skill in the art will recognize that such reaction takes place in conditions that includes a strong base such as sodium t-butoxide (t-BuONa), and solvents such as water and dioxane, under a suitable temperature such as 60° C. to 75° C.

Rxn 9 depicts Boc deprotection under conditions well known in the art such as acid hydrolysis with acids such as TFA and HCl to give compounds such as 7B, 16B, 21A, or 27A.

Rxn 10 depicts acylation using carboxyl acids and coupling reagents or acid chlorides/anhydrides well known in the art to give compounds such as 3A, 16A, 22A, or 28A. An amine can be reacted with an acylating compound, for example, acetic anhydride in a suitable base, such as triethylamine, and a suitable solvent (such as DCM), in a suitable temperature (preferably ambient temperature) to give an acylated compound.

Rxn 11 depicts alkylation using alkyl halides and base to give compounds such as 3A, 9A, 15A, 16A, 20A, 21A, or 22A. A person of ordinary skill in the art will recognize typical such reactions may include S_{N}2 reactions. The reactions may take place in the presence of a base (such as potassium carbonate), and a suitable solvent (such as DMF), at a suitable temperature, preferably at ambient temperature.

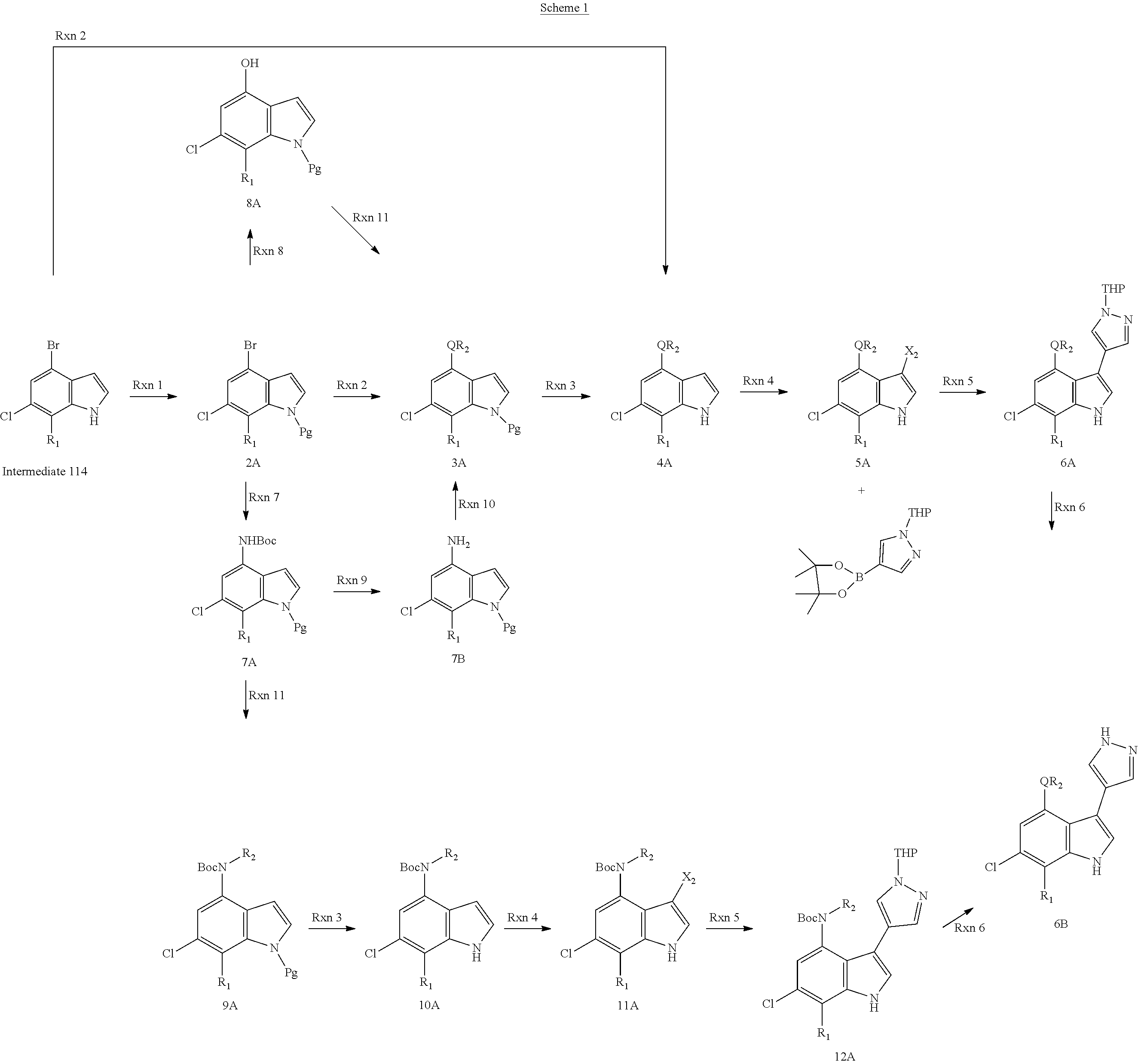

In some embodiments, Intermediate 114 is protected on its ring nitrogen with a suitable protecting group under conditions sufficient to form Formula 2A. Formula 2A is contacted with a palladium catalyst such as XantPhos-Pd-G₂, a base such Cs₂CO₃, and a solvent such as 1,4-dioxane under conditions sufficient to convert into Formula 3A. Subsequently, Formula 3A is deprotected to form Formula 4A. Formula 4A is then contacted with a halogenation reagent such as NB S or NIS in a solvent such as DMF to form Formula 5A with the pyrrole ring halogenated. Formula 5A is contacted with THP-protected pyrazole pinacol boronate via a coupling reaction under conditions sufficient to form Formula 6A. Compound 6A is THP deprotected in the presence of an acid (such as HCl or TFA) to give compound 6B.

In some embodiments, Formula 3A may alternatively be prepared by hydroxylation of aryl bromide. Formula 2A is contacted with a catalyst based on a biarylphosphine ligand tBuBrettPhos or its corresponding palladium precatalyst. The reaction may take place under conditions that include a strong base (such as sodium t-butoxide (t-BuONa)), and solvents (such as water and dioxane), to convert Formula 2A to 8A. Compound 8A then goes through an alkylation reaction using an alkyl halide to give Formula 3A.

In some embodiments, Formula 3A may also be alternatively prepared by cross-coupling Formula 2A with tert-butyl carbamate in the presence of a base such as Cs₂CO₃, a solvent such as 1,4-dioxane or DMF under suitable conditions (such as ambient temperature) to give Formula 7A. Formula 7A is then Boc-deprotected under conditions well known in the art such as acid hydrolysis with acids such as TFA and HCl to give Formula 7B. Formula 7B is then reacted with an acylating compound, for example acetic anhydride in a suitable base such as triethylamine, and a suitable solvent such as DCM to give compound 3A.

Alternatively, in some embodiments, Formula 2A may be contacted with tert-butyl carbamate in the presence of a base (such as Cs₂CO₃), a solvent (such as 1,4-dioxane or DMF) under conditions sufficient to form Formula 7A. Formula 7A has an aniline nitrogen bearing a hydrogen as well as a Boc protecting group. Subsequently, Formula 7A undergoes alkylation on the aniline nitrogen under suitable conditions to form Formula 9A. Formula 9A is contacted with a reagent such as TBAF under conditions sufficient to deprotect the ring nitrogen, but not the aniline nitrogen, to form Formula 10A. After that, Formula 10A is halogenated under conditions sufficient to form Formula 11A. Formula 11A then couples with THP-protected pyrazole pinacol boronate under conditions sufficient to form Formula 12A. Formula 12A is further deprotected on the aniline nitrogen to form Formula 6A.

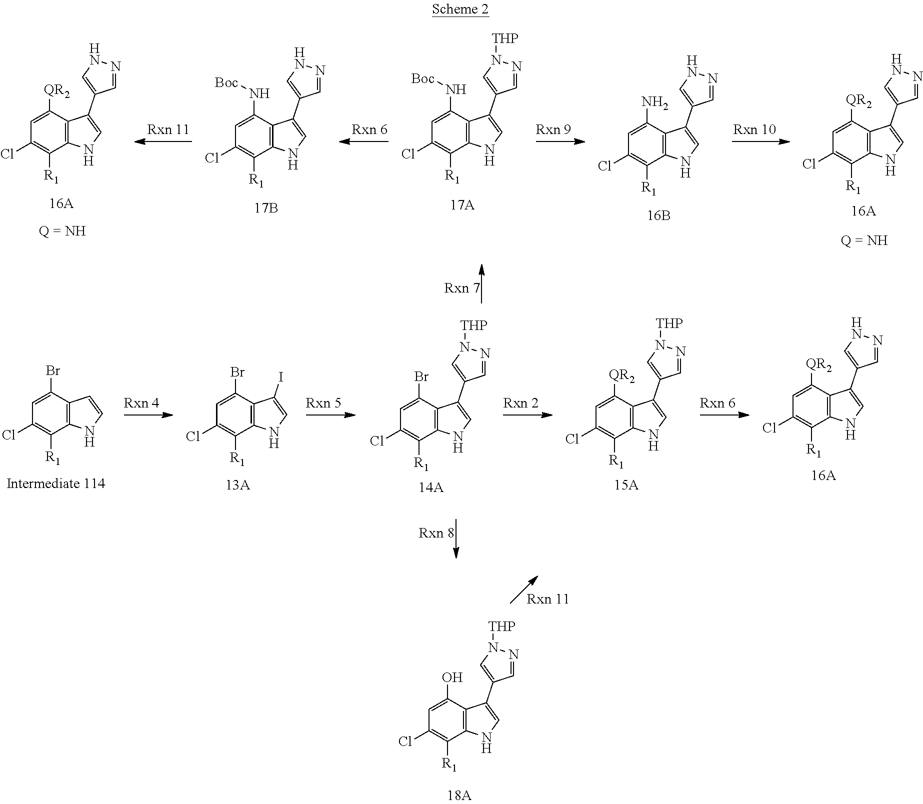

In some embodiments, Intermediate 114 is contacted with a halogenating reagent (such as NBS or NIS) in a solvent such as DMF to give Formula 13A with the pyrrole ring halogenated. Formula 13A is then contacted with THP-protected pyrazole pinacol boronate via a coupling reaction under conditions sufficient to give Formula 14A. Formula 14A is contacted with a palladium catalyst such as XantPhos-Pd-G₂, a base such as Cs₂CO₃, and a solvent such as 1,4-dioxane in a suitable temperature condition sufficient to convert into Formula 15A. Compound 15A is THP deprotected in acidic conditions in the presence of an acid such as HCl or TFA to give compound 16A.

In some embodiments, Formula 15A may alternatively be prepared by hydroxylation of aryl bromide. Formula 15A is contacted with a catalyst based on a biarylphosphine ligand tBuBrettPhos and its corresponding palladium precatalyst. The reaction takes place in a strong base such as sodium t-butoxide (t-BuONa), and solvents such as water and dioxane, under suitable temperature to convert compound 14A to 18A. Compound 18A then goes through an alkylation reaction using an alkyl halide to give compound 15A.

Alternatively, in some embodiments, Formula 14A may be contacted with tert-butyl carbamate in the presence of a base such as Cs₂CO₃, a solvent such as 1,4-dioxane or DMF under conditions sufficient to form Formula 17A. Formula 17A has an aniline nitrogen bearing a hydrogen as well as a Boc protecting group. In some embodiments, Formula 17A is contacted with an acid such as TFA under conditions sufficient to deprotect the ring nitrogen but not the aniline nitrogen to form Formula 17B. Subsequently, Formula 17B undergoes alkylation on the aniline nitrogen under suitable conditions to form Formula 16A.

In further embodiments, Formula 17A is Boc and THP deprotected under conditions well known in the art such as acid hydrolysis with acids such as TFA and HCl to give compound 16B. Formula 16B is then reacted with an acylating compounds, for example acetic anhydride in a suitable base such as triethylamine, and a suitable solvent such as DCM to give Formula 16A.

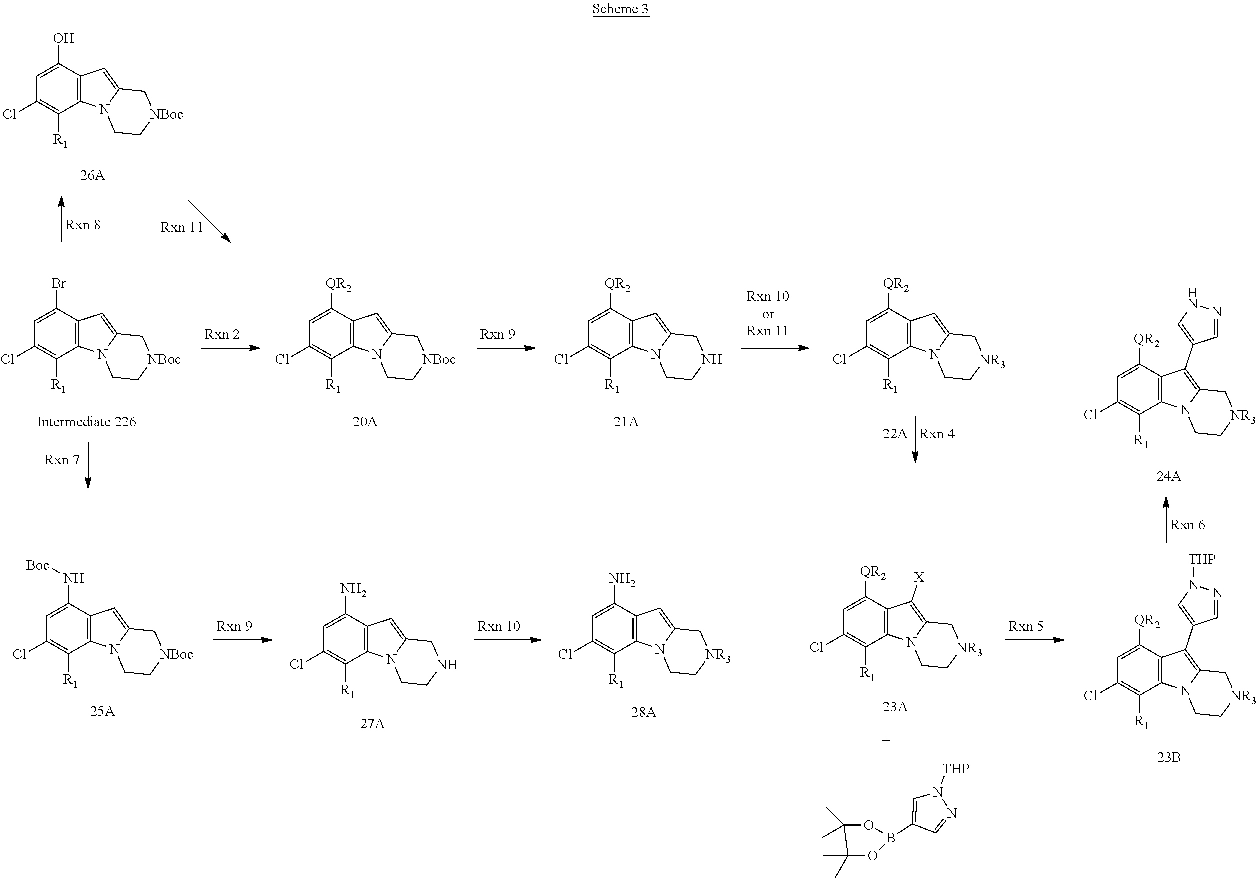

In Scheme 3, Intermediate 226 is contacted with a palladium catalyst such as XantPhos-Pd-G₂, a base such as Cs₂CO₃ and a solvent such as 1,4-dioxane in a suitable temperature condition sufficient to convert into Formula 20A. Formula 20A is then Boc deprotected under conditions well known in the art such as acid hydrolysis with acids such as TFA and HCl to give compound 21A.

In some embodiments, Formula 21A is reacted with an acylating compound, for example acetic anhydride in a suitable base such as triethylamine, and a suitable solvent such as DCM to give compound 22A. Alternatively, Formula 21A undergoes alkylation on the ring nitrogen under suitable conditions to form Formula 22A.

In some embodiments Intermediate 226 is contacted with a catalyst based on a biarylphosphine ligand tBuBrettPhos and its corresponding palladium precatalyst. The reaction takes place in conditions that includes a strong base such as sodium t-butoxide (t-BuONa), and solvents such as water and dioxane, under a suitable temperature to give Formula 26A.

In some other embodiments, Intermediate 226 is cross-coupled with tert-butyl carbamate in the presence of a base such as Cs₂CO₃, a solvent such as 1,4-dioxane or DMF under suitable conditions such as ambient temperature to give compound 25A. Compound 25A is then Boc deprotected under conditions well known in the art such as acid hydrolysis with acids such as TFA and HCl to give compound 27A. Formula 27A undergoes alkylation on the aniline nitrogen under suitable conditions to form Formula 21A. Alternatively, Formula 27A can be reacted with an acylating compounds, for example acetic anhydride in a suitable base such as triethylamine, and a suitable solvent such as DCM to give Formula 28A which is acylated on the ring nitrogen.

In other embodiments, Formula 22A is contacted with a halogenation compound such as NBS or NIS is a solvent such as DMF to form Formula 23A. Formula 23A is then contacted with THP-protected pyrazole pinacol boronate via a coupling reaction under conditions sufficient to form Formula 23B. Compound 23B is THP deprotected in the presence of an acid such as HCl or TFA to give compound 24A.

The compounds of the present disclosure, such as the compounds of Formula Ia, II, IIa, IIc, III, IIIa, IVa, and V, as well as those additional compounds listed in various embodiments, such as those embodiments 1-43, can be prepared according to the above general procedures, particularly in reference to the specific examples that follow. Moreover, a person skilled in the art understands that these general synthesis methods may be modified to adapt to any particular synthesis need. For example, in order to prepare a compound similar to that of Formula 16A but having a tertiary amine (rather than the secondary amine) at the R³ position (see e.g. Formula Ia, II, IIa, IIc, III, IIIa, IVa, and V above), an additional reductive amination step may be used to install the additional alkyl group on Formula 16A. Likewise, compounds having different 5-member ring at the ring A position (see e.g. Formula Ia, IIa, IVa, or V above) may be synthesized via Rxn 5 using a different boronate.

### SCHEME FOR EXAMPLE 1

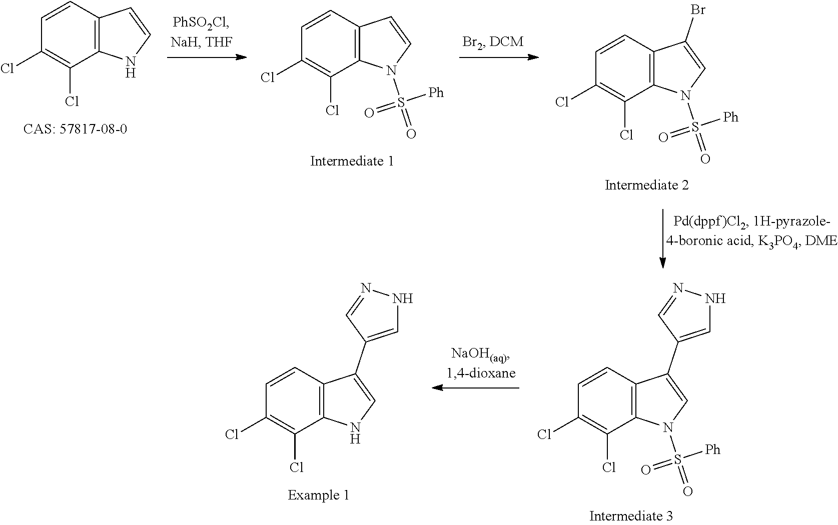 [SMILES: Clc1ccc2cc[nH]c2c1Cl ; O=S(=O)(c1ccccc1)n1ccc2ccc(Cl)c(Cl)c21 ; O=S(=O)(c1ccccc1)n1cc(Br)c2ccc(Cl)c(Cl)c21 ; Clc1ccc2c(-c3cn[nH]c3)c[nH]c2c1Cl ; O=S(=O)(c1ccccc1)n1cc(-c2cn[nH]c2)c2ccc(Cl)c(Cl)c21]

### Intermediate 1

### 1-(Benzenesulfonyl)-6,7-dichloro-indole

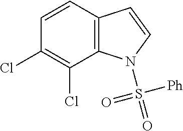 [SMILES: O=S(=O)(c1ccccc1)n1ccc2ccc(Cl)c(Cl)c21]

Dissolve 6,7-dichloro-1H-indole (3.63 g, 19.5 mmol) in THE (100 mL) and add sodium hydride (1.01 g, 25.3 mmol, 60% in mineral oil) portion wise. Stir 10 min (until gas evolution stops) and add benzene sulfonyl chloride (2.74 mL, 21.4 mmol) dropwise. Stir 90 minutes. Add additional sodium hydride (0.31 g, 7.8 mmol, 60% in mineral oil), stir 10 min, and then add more benzene sulfonyl chloride (1.00 mL, 7.82 mmol). Stir 1 hour. Quench with saturated aqueous sodium bicarbonate. Add EtOAc, wash sequentially with water and brine, dry over anhydrous sodium sulfate, filter, and concentrate. Purify by flash column chromatography (EtOAc/hexane) to yield 1-(benzenesulfonyl)-6,7-dichloro-indole (3.0 g, 9.2 mmol, 47% yield). ES-MS (m/z): 326.0/328.0 (M+1).

### Intermediate 2

### 1-(Benzenesulfonyl)-3-bromo-6,7-dichloro-indole

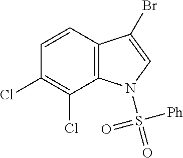 [SMILES: O=S(=O)(c1ccccc1)n1cc(Br)c2ccc(Cl)c(Cl)c21]

Dissolve 1-(benzenesulfonyl)-6,7-dichloro-indole (3.00 g, 9.20 mmol) in DCM (100 mL) and add a solution of bromine (0.52 mL, 10 mmol) in DCM (15 mL). Stir 1 hour at RT. Quench with saturated aqueous sodium thiosulfate and stir vigorously for 15 min (becomes colorless). Dilute with EtOAc, wash with brine, dry over saturated sodium sulfate, filter, and concentrate. Suspend solid in DCM/hexane (ca. 3:1, 20 mL), collect solid, rinsing with additional hexane to yield 1-(benzenesulfonyl)-3-bromo-6,7-dichloro-indole (1.4 g, 3.5 mmol, 38% yield). ES-MS (m/z): 421.0/423.0/425.0 (M+NH₄⁺).

### Intermediate 3

### 1-(Benzenesulfonyl)-6,7-dichloro-3-(1H-pyrazol-4-yl)indole

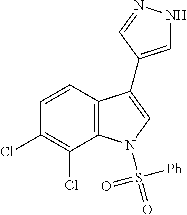 [SMILES: O=S(=O)(c1ccccc1)n1cc(-c2cn[nH]c2)c2ccc(Cl)c(Cl)c21]

Suspend 1-(benzenesulfonyl)-3-bromo-6,7-dichloro-indole (150 mg, 0.370 mmol), 1H-pyrazole-4-boronic acid (0.25 g, 2.2 mmol) and Pd(dppf)Cl₂ (71 mg, 0.092 mmol) in 1,2-dimethoxyethane (2.2 mL, 21 mmol) in a microwave vial. Add aqueous K₃PO₄ (1.1 mL, 1.1 mmol, 1 M), sparge with N₂, seal vial, and heat to 80° C. for 1 hour. Cool to RT, dilute with EtOAc, wash sequentially with saturated aqueous sodium bicarbonate and brine, dry over sodium sulfate, filter, and concentrate. Purify by flash column chromatography (EtOAc/hexane) to yield 1-(benzenesulfonyl)-6,7-dichloro-3-(1H-pyrazol-4-yl)indole (100 mg, 0.255 mmol, 69% yield). ES-MS (m/z): 392.2/394.2 (M+1).

### EXAMPLE 1

### 6,7-Dichloro-3-(1H-pyrazol-4-yl)-1H-indole

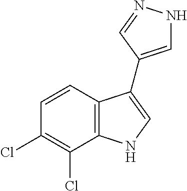 [SMILES: Clc1ccc2c(-c3cn[nH]c3)c[nH]c2c1Cl]

Suspend 1-(benzenesulfonyl)-6,7-dichloro-3-(1H-pyrazol-4-yl)indole (100 mg, 0.2549 mmol) in 1,4-dioxane (4 mL, 46.7 mmol). Add 2.5 M aqueous sodium hydroxide (1.7 mL, 4.3 mmol) and heat to 80° C. overnight. Cool to room temperature, dilute with EtOAc, wash 3 times with saturated aqueous sodium bicarbonate, once with brine, dry over anhydrous sodium sulfate, filter and concentrate. Purify by column chromatography (MeOH/DCM) to yield 6,7-dichloro-3-(1H-pyrazol-4-yl)-1H-indole (16.7 mg, 0.0662 mmol, 26.0% yield) as a light-yellow solid. ES-MS (m/z): 252.0/254.0 (M+1). ¹H NMR (400 MHz, DMSO): 12.94-12.88 (s, 1H), 11.67 (s, 1H), 8.15 (s, 1H), 7.93-7.89 (s, 1H), 7.80 (d, J=8.6 Hz, 1H), 7.70 (d, J=2.5 Hz, 1H), 7.24 (d, J=8.6 Hz, 1H).

### SCHEME FOR EXAMPLE 2

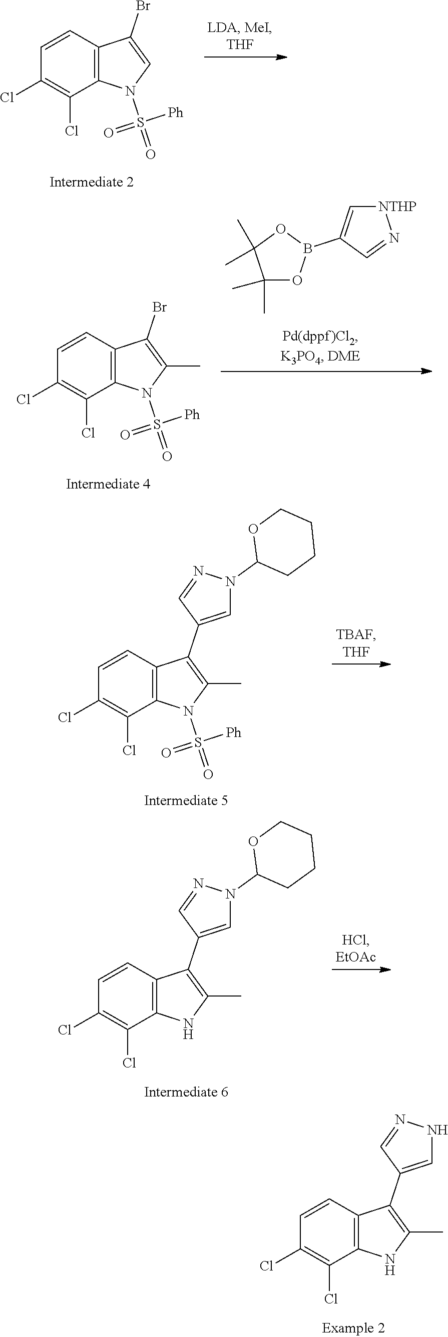

### Intermediate 4

### 1-(Benzenesulfonyl)-3-bromo-6,7-dichloro-2-methyl-indole

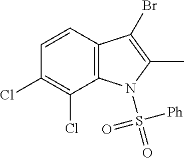 [SMILES: Cc1c(Br)c2ccc(Cl)c(Cl)c2n1S(=O)(=O)c1ccccc1]

Dissolve diisopropylamine (0.14 mL, 0.96 mmol) in anhydrous THF (5.0 mL), cool to −78° C., then add n-butyllithium (1.6 M in hexanes, 0.58 mL, 0.93 mmol) dropwise. Warm mixture to 0° C. In a separate flask, dissolve 1-(benzenesulfonyl)-3-bromo-6,7-dichloro-indole (300 mg, 0.74 mmol) in anhydrous THF (5.0 mL) and cool to −78° C. Add LDA solution dropwise to second flask, then warm to 0° C. Add iodomethane (0.10 mL, 1.7 mmol) and allow to warm to ambient temperature. Stir 90 minutes, then quench with saturated aqueous sodium bicarbonate. Extract with EtOAc. Wash once with brine, dry over anhydrous sodium sulfate, filter and concentrate. Purify by column chromatography (EtOAc/hexanes) to yield 1-(benzenesulfonyl)-3-bromo-6,7-dichloro-2-methyl-indole (0.27 g, 0.64 mmol, 87% yield) as a white solid. ¹H NMR (400 MHz, DMSO): 7.89-7.87 (m, 2H), 7.80-7.76 (m, 1H), 7.68-7.62 (m, 3H), 7.41 (d, J=8.4 Hz, 1H), 2.59 (s, 3H).

### Intermediate 5

### 1-(Benzenesulfonyl)-6,7-dichloro-2-methyl-3-(1-tetrahydropyran-2-ylpyrazol-4-yl)indole

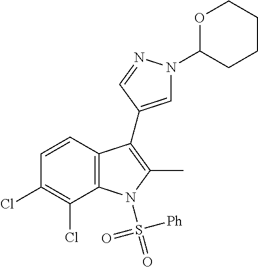 [SMILES: Cc1c(-c2cnn(C3CCCCO3)c2)c2ccc(Cl)c(Cl)c2n1S(=O)(=O)c1ccccc1]

Dissolve 1-(benzenesulfonyl)-3-bromo-6,7-dichloro-2-methyl-indole (140 mg, 0.33 mmol), 1-tetrahydropyran-2-yl-4-(4,4,5,5-tetramethyl-1,3,2-dioxaborolan-2-yl)pyrazole (0.28 g, 1.00 mmol) and Pd(dppf)Cl₂ (100 mg, 0.13 mmol, 95 mass %) in 1,2-dimethoxyethane (2.2 mL). Add aqueous K₃PO₄ (1.0 mL, 1.00 mmol, 1 M), sparge with N₂, seal vial and heat to 80° C. for 18 hours. Cool to RT, dilute with EtOAc, filter through diatomaceous earth. Wash filtrate sequentially with saturated aqueous sodium bicarbonate and brine, dry over sodium sulfate, filter and concentrate. Purify by flash column chromatography (acetone/hexane) to yield 1-(benzenesulfonyl)-6,7-dichloro-2-methyl-3-(1-tetrahydropyran-2-ylpyrazol-4-yl)indole (0.16 g, 0.33 mmol, 100% yield) as a colorless oil. ES-MS (m/z): 490.2/492.2 (M+1).

### Intermediate 6

### 6,7-Dichloro-2-methyl-3-(1-tetrahydropyran-2-ylpyrazol-4-yl)-1H-indole

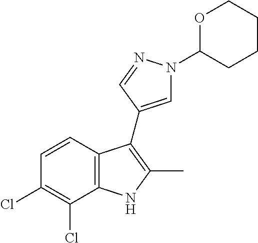 [SMILES: Cc1[nH]c2c(Cl)c(Cl)ccc2c1-c1cnn(C2CCCCO2)c1]

Dissolve 1-(benzenesulfonyl)-6,7-dichloro-2-methyl-3-(1-tetrahydropyran-2-ylpyrazol-4-yl)indole (0.16 g, 0.33 mmol) in anhydrous THF (5.0 mL). Add TBAF (1.0M in THF, 1.7 mL, 1.7 mmol) and stir at ambient temperature 18 hours. Dilute with ethyl acetate, wash sequentially with 1:1 water/saturated aqueous sodium bicarbonate and brine, dry over anhydrous sodium sulfate, filter and concentrate. Purify by column chromatography (acetone/hexanes) to yield 6,7-dichloro-2-methyl-3-(1-tetrahydropyran-2-ylpyrazol-4-yl)-1H-indole (80 mg, 0.23 mmol, 66% yield) as a colorless foam. ES-MS (m/z): 350.0/352.0 (M+1).

### EXAMPLE 2

### 6,7-Dichloro-2-methyl-3-(1H-pyrazol-4-yl)-1H-indole

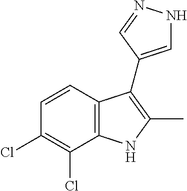 [SMILES: Cc1[nH]c2c(Cl)c(Cl)ccc2c1-c1cn[nH]c1]

Dissolve 6,7-dichloro-2-methyl-3-(1-tetrahydropyran-2-ylpyrazol-4-yl)-1H-indole (80 mg, 0.23 mmol) in EtOAc (5.0 mL). Add aqueous hydrochloric acid (37%, 0.5 mL) and stir at ambient temperature for 90 minutes. Dilute with EtOAc, wash sequentially with saturated aqueous sodium bicarbonate and brine, dry over anhydrous sodium sulfate, filter, and concentrate. Purify by column chromatography (acetone/hexanes) to yield. ES-MS (m/z): 266.0/268.0. (M+1). ¹H NMR (400 MHz, DMSO): 13.03-13.01 (s, 1H), 11.50 (s, 1H), 8.02-7.93 (s, 1H), 7.81-7.79 (s, 1H), 7.54 (d, J=8.4 Hz, 1H), 7.17 (d, J=8.5 Hz, 1H), 2.49 (s, 3H).

### SCHEME FOR EXAMPLE 3

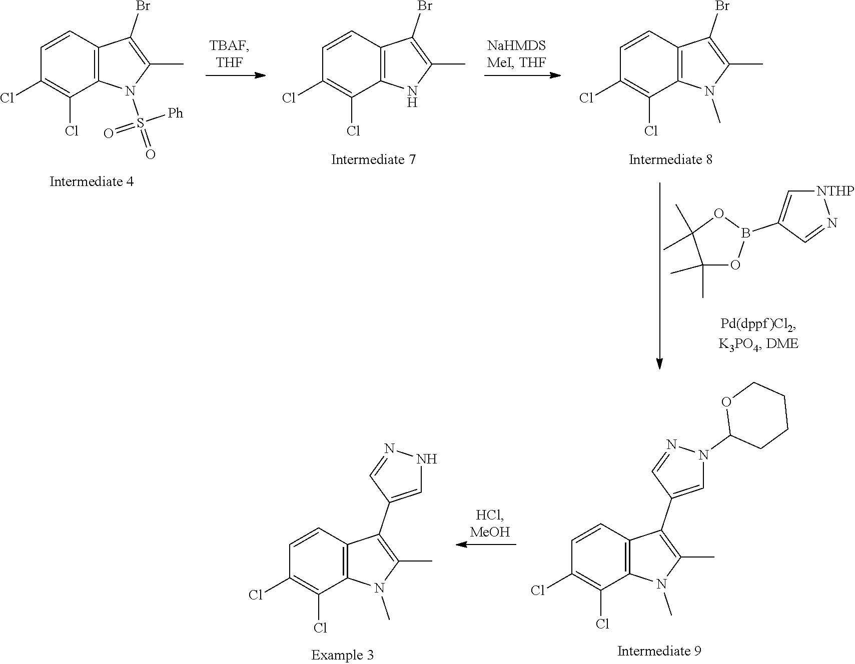

### Intermediate 7

### 3-Bromo-6,7-dichloro-2-methyl-1H-indole

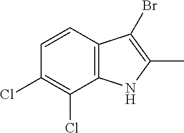

Dissolve 1-(benzenesulfonyl)-3-bromo-6,7-dichloro-2-methyl-indole (0.52 g, 1.2 mmol) in anhydrous THF (12 mL). Add TBAF (1.0M in THF, 6.2 mL, 6.2 mmol) and stir at ambient temperature 18 hours. Dilute with EtOAc, wash sequentially with 1:1 water/saturated aqueous sodium bicarbonate and brine, dry over anhydrous sodium sulfate, filter and concentrate. Purify by column chromatography (acetone/hexanes) to yield 3-bromo-6,7-dichloro-2-methyl-1H-indole (0.34 g, 0.12 mmol, 98% yield) as a tan solid. ES-MS (m/z): 275.8/277.8/279.8 (M−1, negative ionization mode).

### Intermediate 8

### 3-Bromo-6,7-dichloro-1,2-dimethyl-indole

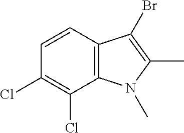

Dissolve 3-bromo-6,7-dichloro-2-methyl-1H-indole (0.34 g, 0.12 mmol) in anhydrous THF (6 mL). Cool to 0° C. and add sodium bis(trimethylsilyl)amide (1.0 M in THF, 1.3 mL, 1.3 mmol). Stir 15 minutes, add iodomethane (0.11 mL, 1.8 mmol) and stir at 0° C. for 1 hour. Quench with aqueous saturated sodium bicarbonate and warm to ambient temperature. Dilute with EtOAc, wash sequentially with saturated aqueous sodium bicarbonate and brine, dry over anhydrous sodium sulfate, filter, and concentrate. Purify by column chromatography (MTBE/hexanes) to yield 3-bromo-6,7-dichloro-1,2-dimethyl-indole (0.26 g, 0.89 mmol, 73% yield).

### Intermediate 9

### 6,7-Dichloro-1,2-dimethyl-3-(1-tetrahydropyran-2-ylpyrazol-4-yl)indole

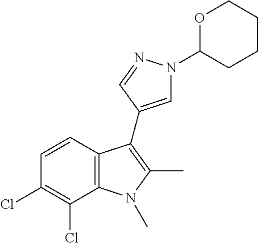 [SMILES: Cc1c(-c2cnn(C3CCCCO3)c2)c2ccc(Cl)c(Cl)c2n1C]

Dissolve 3-bromo-6,7-dichloro-1,2-dimethyl-indole (260 mg, 0.89 mmol), 1-tetrahydropyran-2-yl-4-(4,4,5,5-tetramethyl-1,3,2-dioxaborolan-2-yl)pyrazole (0.74 g, 2.66 mmol) and Pd(dppf)Cl₂ (270 mg, 0.35 mmol, 95 mass %) in 1,2-dimethoxyethane (6.0 mL). Add aqueous K₃PO₄ (2.6 mL, 2.60 mmol, 1 mol/L), sparge with N2, seal vial and heat to 80° C. for 18 hours. Cool to RT, dilute with EtOAc and filter through diatomaceous earth. Wash filtrate sequentially with saturated aqueous sodium bicarbonate and brine, dry over sodium sulfate, filter and concentrate. Purify by flash column chromatography (acetone/hexane) to yield 6,7-dichloro-1,2-dimethyl-3-(1-tetrahydropyran-2-ylpyrazol-4-yl)indole (41 mg, 0.11 mmol, 13% yield) as a colorless oil. ES-MS (m/z): 364.0/366.0 (M+1).

### EXAMPLE 3

### 6,7-Dichloro-1,2-dimethyl-3-(1H-pyrazol-4-yl)indole

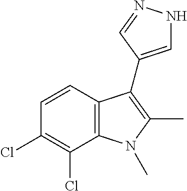 [SMILES: Cc1c(-c2cn[nH]c2)c2ccc(Cl)c(Cl)c2n1C]

Dissolve 6,7-dichloro-1,2-dimethyl-3-(1-tetrahydropyran-2-ylpyrazol-4-yl)indole (41 mg, 0.11 mmol) in anhydrous 1,4-dioxane (2 mL) and MeOH (1.0 mL). Add hydrochloric acid (4 N in 1,4-dioxane) and stir at ambient temperature for 18 hours. Dilute with EtOAc, wash sequentially with saturated aqueous sodium bicarbonate and brine, dry over anhydrous sodium sulfate, filter and concentrate. Purify by column chromatography (acetone/hexanes) to yield 6,7-dichloro-1,2-dimethyl-3-(1H-pyrazol-4-yl)indole (29 mg, 0.10 mmol, 92% yield) as a white solid. ES-MS (m/z): 279.6/281.6 (M+1). ¹H NMR (400 MHz, DMSO): 13.09-13.07 (s, 1H), 7.98-7.96 (s, 1H), 7.76-7.74 (s, 1H), 7.46 (d, J=8.4 Hz, 1H), 7.20 (d, J=8.4 Hz, 1H), 4.07 (s, 3H), 2.43 (s, 3H).

### SCHEME FOR EXAMPLE 4

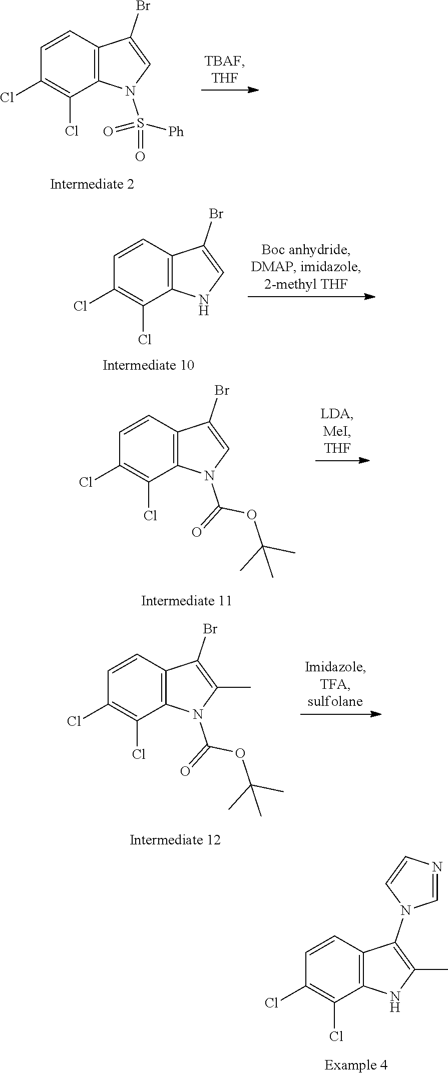 [SMILES: O=S(=O)(c1ccccc1)n1cc(Br)c2ccc(Cl)c(Cl)c21 ; Clc1ccc2c(Br)c[nH]c2c1Cl ; CC(C)(C)OC(=O)n1cc(Br)c2ccc(Cl)c(Cl)c21 ; Cc1c(Br)c2ccc(Cl)c(Cl)c2n1C(=O)OC(C)(C)C ; Cc1[nH]c2c(Cl)c(Cl)ccc2c1-n1ccnc1]

### Intermediate 10

### 3-Bromo-6,7-dichloro-1H-indole

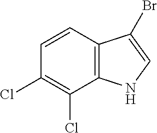 [SMILES: Clc1ccc2c(Br)c[nH]c2c1Cl]

Dissolve 1-(benzenesulfonyl)-3-bromo-6,7-dichloro-indole (0.77 g, 1.9 mmol) in anhydrous 1,4-dioxane (25 mL). Add TBAF (1.0 M in THF, 9.5 mL, 9.5 mmol) and stir at ambient temperature for 30 min. Dilute with EtOAc, wash sequentially with water, saturated aqueous sodium bicarbonate and brine, dry over anhydrous sodium sulfate, filter, and concentrate. Purify by column chromatography (acetone/hexanes) to yield 3-bromo-6,7-dichloro-1H-indole (0.50 g, 1.9 mmol, 100% yield) as a tan solid. ES-MS (m/z): 261.8/263.8/265.8 (M−1, negative ionization mode).

### Intermediate 11

### tert-Butyl 3-bromo-6,7-dichloro-indole-1-carboxylate

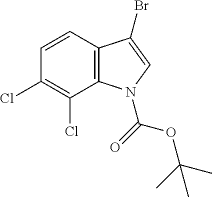 [SMILES: CC(C)(C)OC(=O)n1cc(Br)c2ccc(Cl)c(Cl)c21]

Dissolve 3-bromo-6,7-dichloro-1H-indole (0.50 g, 1.9 mmol) in anhydrous 2-methyltetrahydrofuran (10 mL). Add 2-dimethylaminopyridine (47 mg, 0.38 mmol) followed by tert-butoxycarbonyl tert-butyl carbonate (0.49 mL, 2.1 mmol). Stir at ambient temperature 18 hours. Add imidazole (50 mg, 0.73 mmol) and stir 10 min at ambient temperature. Dilute with EtOAc, wash twice with saturated aqueous ammonium chloride followed by brine, dry over anhydrous sodium sulfate, filter, and concentrate. Purify by column chromatography (MTBE/hexanes) to yield tert-butyl 3-bromo-6,7-dichloro-indole-1-carboxylate (0.56 g, 1.5 mmol, 81% yield) as a colorless oil. ¹H NMR (400 MHz, DMSO): 8.08 (s, 1H), 7.64 (d, J=8.4 Hz, 1H), 7.52 (d, J=8.5 Hz, 1H), 1.61 (s, 9H).

### Intermediate 12

### tert-Butyl 3-bromo-6,7-dichloro-2-methyl-indole-1-carboxylate

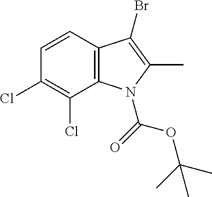 [SMILES: Cc1c(Br)c2ccc(Cl)c(Cl)c2n1C(=O)OC(C)(C)C]

Dissolve diisopropylamine (0.28 mL, 2.0 mmol) in anhydrous THF (5.0 mL) and cool to −78° C. Add n-butyllithium (2.5 M in hexanes, 0.53 mL, 1.9 mmol) dropwise. Warm mixture to 0° C. In a separate flask, dissolve tert-butyl 3-bromo-6,7-dichloro-indole-1-carboxylate (0.56 g, 1.5 mmol) in anhydrous THF (5.0 mL) and cool to −78° C. Add LDA solution dropwise to second flask. Stir 5 min, then add iodomethane (0.19 mL, 3.1 mmol) and allow to warm to ambient temperature. Stir 3 hours, quench with saturated aqueous sodium bicarbonate, add EtOAc, and extract. Wash once with brine, dry over anhydrous sodium sulfate, filter, and concentrate. Purify by column chromatography (ethyl acetate/hexanes) to yield tert-butyl 3-bromo-6,7-dichloro-2-methyl-indole-1-carboxylate (0.56 g, 1.5 mmol, 96% yield) as a colorless oil. ¹H NMR (400 MHz, DMSO): 7.54 (d, J=8.5 Hz, 1H), 7.42 (d, J=8.5 Hz, 1H), 2.48 (s, 3H), 1.62 (s, 9H).

### EXAMPLE 4

### 6,7-Dichloro-3-imidazol-1-yl-2-methyl-1H-indole

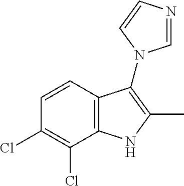 [SMILES: Cc1[nH]c2c(Cl)c(Cl)ccc2c1-n1ccnc1]

In a microwave vial, combine tert-butyl 3-bromo-6,7-dichloro-2-methyl-indole-1-carboxylate (0.12 g, 0.32 mmol), sulfolane (1.0 mL), imidazole (0.43 g, 6.3 mmol), and TFA (0.12 mL, 1.6 mmol). Seal the vial and heat to 160° C. for 18 hours. Cool to ambient temperature, vent with a needle and open the vial. Dilute with EtOAc, wash sequentially with water and brine, dry over anhydrous sodium sulfate, filter, and concentrate. Purify by column chromatography (acetone/hexanes). Suspend recovered product in dichloromethane (2 mL) and collect by suction filtration, rinsing with DCM (2 mL). Yields 6,7-dichloro-3-imidazol-1-yl-2-methyl-1H-indole (30 mg, 0.11 mmol, 35% yield) as a tan solid. ES-MS (m/z): 266.0/268.0 (M+1). ¹H NMR (400 MHz, DMSO): 11.95-11.90 (s, 1H), 7.86 (m, 1H), 7.42 (m, 1H), 7.26-7.21 (m, 2H), 7.15 (m, 1H), 2.36 (s, 3H).

### SCHEME FOR EXAMPLE 5

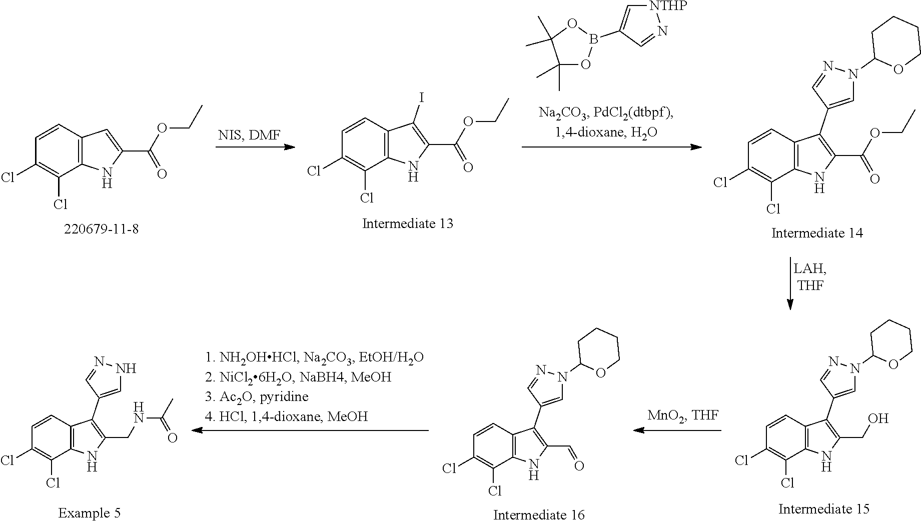

### Intermediate 13

### Ethyl 6,7-dichloro-3-iodo-1H-indole-2-carboxylate

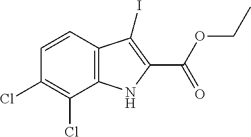 [SMILES: CCOC(=O)c1[nH]c2c(Cl)c(Cl)ccc2c1I]

Dissolve ethyl 6,7-dichloro-1H-indole-2-carboxylate (78 g, 0.30 mol) in DMF (0.78 L) and add a solution of N-iodosuccinimide (81.6 g, 0.36 mol) in DMF (0.8 L, 10 vol) at 0° C. Stir for 1 hour at RT. Monitor reaction by TLC. When complete, dilute with saturated sodium hyposulfite solution (500 mL) at 0° C. Filter the resulting solid and dry under vacuum to give crude ethyl 6,7-dichloro-3-iodo-1H-indole-2-carboxylate. Repeat reaction on identical scale and combine both crude lots. Triturate with deithylether:heptane (1:1, 200 mL). Collect the solid by filtration and dry under vacuum to give 6,7-dichloro-3-iodo-1H-indole-2-carboxylate (152 g, 65%) as a white solid. ES-MS (m/z): 384.00/386.00 (M+1).

### Intermediate 14

### Ethyl 6,7-dichloro-3-(1-tetrahydropyran-2-ylpyrazol-4-yl)-1H-indole-2-carboxylate

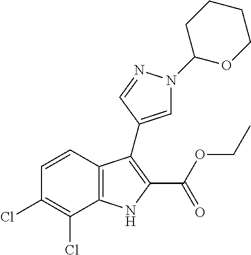 [SMILES: CCOC(=O)c1[nH]c2c(Cl)c(Cl)ccc2c1-c1cnn(C2CCCCO2)c1]

Dissolve ethyl 6,7-dichloro-3-iodo-1H-indole-2-carboxylate (78 g, 0.20 mol) in 1, 4-dioxane (1.17 L, 15 vol) and add 1-(tetrahydro-2H-pyran-2-yl)-4-(4,4,5,5-tetramethyl-1,3,2-dioxaborolan-2-yl)-1H-pyrazole (169.5 g, 0.61 mol) and sodium carbonate (64.6 g, 0.61 mol) at RT. Purge under an argon atmosphere for 10 min. Add PdCl₂(dtbpf) (26.47 g, 0.04 mol) at RT. Heat to 90° C. and stir for 2 hours. Monitor reaction by TLC. Once complete, dilute with water (500 mL) and extract with ethyl acetate (2×500 mL). Combine the organic extracts and wash with brine (250 mL), dry over anhydrous sodium sulfate, filter and concentrate under reduced pressure to obtain the crude ethyl 6,7-dichloro-3-(1-tetrahydropyran-2-ylpyrazol-4-yl)-1H-indole-2-carboxylate. Purify by chromatography using (EtOAc/heptane) and triturate with n-heptane (200 mL). Collect solids by filtration and dry under vacuum to give ethyl 6,7-dichloro-3-(1-tetrahydropyran-2-ylpyrazol-4-yl)-1H-indole-2-carboxylate (66 g, 80%) as an off-white solid. ES-MS (m/z): 408.20/410.20 (M+1).

### Intermediate 15

### [6,7-Dichloro-3-(1-tetrahydropyran-2-ylpyrazol-4-yl)-1H-indol-2-yl]methanol

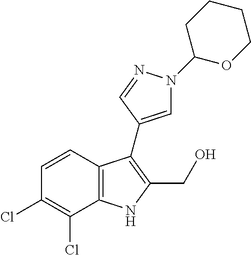 [SMILES: OCc1[nH]c2c(Cl)c(Cl)ccc2c1-c1cnn(C2CCCCO2)c1]

Dissolve ethyl 6,7-dichloro-3-(1-tetrahydropyran-2-ylpyrazol-4-yl)-1H-indole-2-carboxylate (10.0 g, 0.0245 mol) in anhydrous THF (150 mL). Cool the solution in an ice bath for 30 minutes. Treat the solution with lithium aluminum hydride (16.0 mL, 0.032 mol, 2 M in THF). Allow to slowly warm to RT and stir for 18 hours. Cool with an ice bath. Quench the reaction sequentially with water (1.50 mL), 5 N aqueous NaOH (3.0 mL) and water (10.0 mL). Filter solids through a pad of diatomaceous earth. Wash the filtrate sequentially with water (3×) and brine (1×), dry with magnesium sulfate, filter, and concentrate under reduced pressure to obtain [6,7-dichloro-3-(1-tetrahydropyran-2-ylpyrazol-4-yl)-1H-indol-2-yl]methanol (8.50 g, 95%) as a pale tan solid. ES-MS (m/z): 365.6, 367.6 (M+1).

### Intermediate 16

### 6,7-Dichloro-3-(1-tetrahydropyran-2-ylpyrazol-4-yl)-1H-indole-2-carbaldehyde

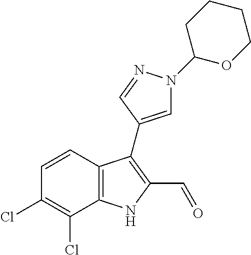 [SMILES: O=Cc1[nH]c2c(Cl)c(Cl)ccc2c1-c1cnn(C2CCCCO2)c1]

Dissolve [6,7-dichloro-3-(1-tetrahydropyran-2-ylpyrazol-4-yl)-1H-indol-2-yl]methanol (8.50 g, 0.0232 mol) in anhydrous THF (150 mL). Add manganese dioxide (30.0 g, 1.84 mol) and stir vigorously for 3 hours. Filter the mixture through a pad of diatomaceous earth. Concentrate the solution to give 6,7-dichloro-3-(1-tetrahydropyran-2-ylpyrazol-4-yl)-1H-indole-2-carbaldehyde (7.83 g, 93%) as a pale-yellow solid. ES-MS (m/z): 363.6/365.6 (M+1).

### EXAMPLE 5

### N-[[6,7-Dichloro-3-(1H-pyrazol-4-yl)-1H-indol-2-yl]methyl]acetamide

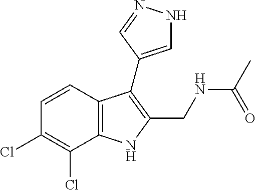 [SMILES: CC(=O)NCc1[nH]c2c(Cl)c(Cl)ccc2c1-c1cn[nH]c1]

Add 6,7-Dichloro-3-(1-tetrahydropyran-2-ylpyrazol-4-yl)-1H-indole-2-carbaldehyde (0.15 g, 0.41 mmol), hydroxylamine hydrochloride (0.057 g, 0.82 mmol) and sodium carbonate (0.065 g, 0.62 mmol) to ethanol (0.69 mL) and water (0.26 mL) in a screw cap vial at RT. Stir at 60° C. for 1 hour. Quench with water and extract with EtOAc (2×), dry combined organic layers with magnesium sulfate and concentrate under reduced pressure. Add methanol to the crude residue and cool to 0° C. Add nickel(II) chloride hexahydrate (0.053 g, 0.41 mmol) and sodium borohydride (0.093 g, 0.41 mmol) in three portions. Stir for one hour at RT. Quench with water and extract with EtOAc (2×). Wash the combined organic extracts with brine (1×), dry with magnesium sulfate and concentrate under reduced pressure. Add the residue, acetic anhydride (0.043 mL, 0.45 mmol) and pyridine (0.050 mL, 0.62 mmol) to DCM (4.0 mL) at 0° C. Stir the reaction for 1 hour. Dilute with toluene and concentrate under reduced pressure. Purify by flash chromatography (EtOAc/hexanes) to afford N-[[6,7-di chloro-3-(1-tetrahydropyran-2-ylpyrazol-4-yl)-1H-indol-2-yl]methyl]acetamide (96.8 mg, 59% yield). Add N-[[6,7-dichloro-3-(1-tetrahydropyran-2-ylpyrazol-4-yl)-1H-indol-2-yl]methyl]acetamide (67 mg, 0.164 mmol) and 4.0 M hydrochloric acid in 1,4-dioxane to methanol and 1,4-dioxane to a screw cap vial. Stir the reaction for 1 hour at RT. Concentrate the reaction under reduced pressure and dilute with ethyl acetate. Wash the organic layer with saturated aqueous sodium bicarbonate. Dry the organic layer with magnesium sulfate, filter and concentrate under vacuum. Purify by flash chromatography (EtOAc/MeOH) to give N-[[6,7-dichloro-3-(1H-pyrazol-4-yl)-1H-indol-2-yl]methyl]acetamide (39.8 mg, 73% yield): ES-MS (m/z) 322.8, 324.8 (M+1). ¹H NMR (400 MHz, DMSO): 12.98 (brs, 1H), 11.45 (s, 1H), 8.22 (s, 1H), 7.90 (brs, 2H), 7.60 (d, 1H, J=8.39 Hz), 7.23 (d, 1H, J=8.30 Hz), 4.47 (d, 2H, J=5.15 Hz), 1.87 (s, 3H).

### SCHEME FOR EXAMPLE 6

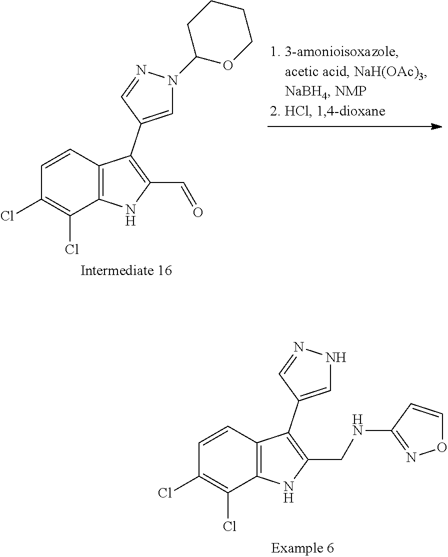 [SMILES: O=Cc1[nH]c2c(Cl)c(Cl)ccc2c1-c1cnn(C2CCCCO2)c1 ; Clc1ccc2c(-c3cn[nH]c3)c(CNc3ccon3)[nH]c2c1Cl]

### EXAMPLE 6

### N-[[6,7-Dichloro-3-(1H-pyrazol-4-yl)-1H-indol-2-yl]methyl]isoxazol-3-amine

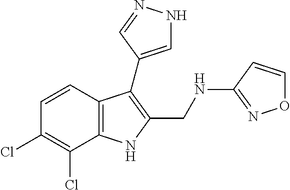 [SMILES: Clc1ccc2c(-c3cn[nH]c3)c(CNc3ccon3)[nH]c2c1Cl]

Add 6,7-dichloro-3-(1-tetrahydropyran-2-ylpyrazol-4-yl)-1H-indole-2-carbaldehyde (0.20 g, 0.55 mmol), 3-aminoisoxazole (0.14 g, 1.65 mmol), 1-methyl-2-pyrrolidinone (2.75 mL), and acetic acid (0.19 mL, 3.29 mmol) to a screw cap vial at room temperature for 16 hours. Treat with sodium triacetoxyborohydride (0.35 g, 1.65 mmol). Stir for 7 hours. Treat with more triacetoxyborohydride (0.35 g, 1.65 mmol). Stir for 16 hours. Treat with sodium borohydride (0.20 g, 0.529 mmol). Stir for 20 minutes. Dilute with water. Extract with EtOAc (2×) and concentrate under reduced pressure. Dissolve in 1,4-dioxane (1.50 mL) and add concentrated hydrochloric acid (0.13 mL). Stir for 16 hours. Concentrate under reduced pressure. Purify by reverse phase chromatography to give N-[[6,7-dichloro-3-(1H-pyrazol-4-yl)-1H-indol-2-yl]methyl]isoxazol-3-amine (72.6 mg, 38% yield). ES-MS (m/z): 348.0, 350.0 (M+1). ¹H NMR (400 MHz, DMSO): 12.91 (bs, 1H), 11.66 (bs, 1H), 8.42 (d, 1H, J=1.85 Hz), 7.88 (s, 2H), 7.61 (d, 1H, J=8.66 Hz), 7.22 (d, 1H, J=8.66 Hz), 6.54 (m, 1H), 5.98 (d, 1H, J=1.85 Hz), 4.44 (d, 2H, J=5.35 Hz).

### SCHEME FOR EXAMPLE 7

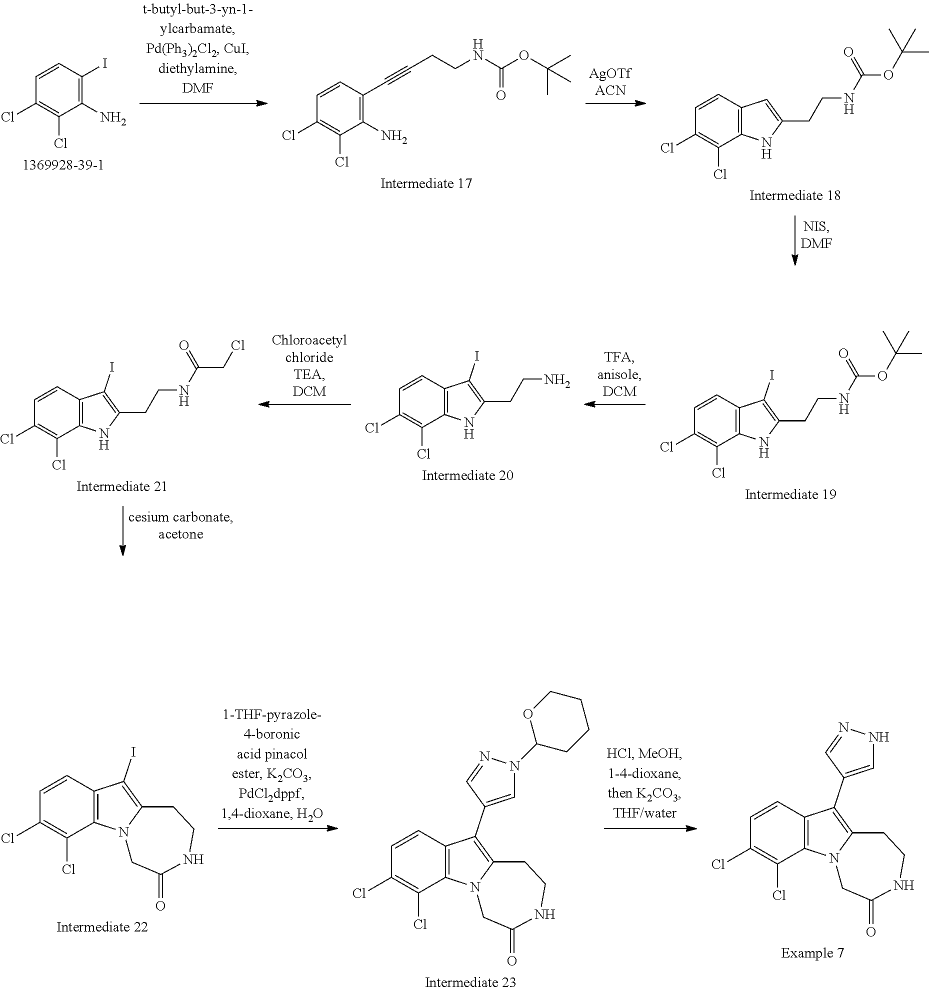

### Intermediate 17

### tert-Butyl N-[4-(2-amino-3,4-dichloro-phenyl)but-3-ynyl)]carbamate

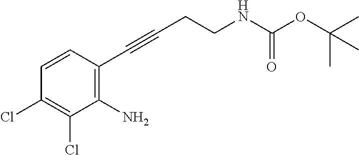 [SMILES: CC(C)(C)OC(=O)NCCC#Cc1ccc(Cl)c(Cl)c1N]

Add diethylamine (5.40 mL, 52.0 mmol) to 2,3-dichloro-6-iodoaniline (10.0 g, 34.7 mmol), bis(triphenylphosphine)palladium (11) dichloride (0.73 g, 1.04 mmol) and cuprous iodide (0.33 g, 1.73 mmol) in N, N-dimethylformamide (150 mL) with stirring, under nitrogen at RT. Sparge with a stream of nitrogen, add tert-butyl but-3-yn-1-ye carbamate (7.20 g, 41.0 mmol) and heat to 50° C. with stirring and under nitrogen for 18 hours. After 18 hours, cool the reaction and pour into water. Extract with EtOAc (×3) and wash the combined organic layers sequentially with 1N hydrochloric acid, water and brine. Dry over anhydrous magnesium sulfate, filter, and concentrate. Purify the crude by flash chromatography (EtOAc/Hexanes) to yield tert-butyl N-[4-(2-amino-3,4-dichloro-phenyl) but-3-ynyl]carbamate (10.7 g, 32.5 mmol, 94%). ES-MS (m/z): 228.6/230.6 (M+1, −Boc).

### Intermediate 18

### tert-Butyl N-[2-(6,7-dichloro-1H-indol-2-yl)ethyl]carbamate

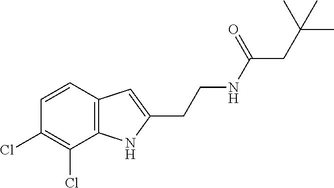 [SMILES: CC(C)(C)CC(=O)NCCc1cc2ccc(Cl)c(Cl)c2[nH]1]

Add silver triflate (0.80 g, 3.11 mmol) to tert-butyl N-[4-(2-amino-3,4-dichloro-phenyl)but-3-ynyl]carbamate (10 g, 30.4 mmol) in ACN (100 mL) with stirring, under nitrogen at RT. Heat the reaction to reflux overnight. Cool to RT and concentrate under reduced pressure. Pour into water and extract with EtOAc (×3). Wash combined organic extracts with brine, dry over anhydrous magnesium sulfate, filter, and concentrate. Purify by flash chromatography (EtOAc/Hex) to yield tert-butyl N-[2-(6,7-dichloro-1H-indol-2-yl) ethyl] carbamate (8.57 g, 26.0 mmol, 86%). ES-MS (m/z): 328.6/330.6 (M+1).

### Intermediate 19

### tert-Butyl N-[2-(6,7-dichloro-3-iodo-1H-indol-2-yl)ethyl]carbamate

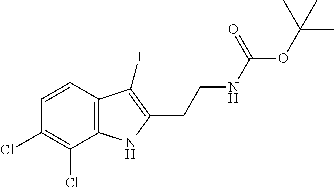 [SMILES: CC(C)(C)OC(=O)NCCc1[nH]c2c(Cl)c(Cl)ccc2c1I]

Add N-iodosuccinimide (0.93 g, 4.02 mmol) to tert-butyl N-[2-(6,7-dichloro-1H-indol-2-yl) ethyl] carbamate (1.26 g, 3.83 mmol) in DMF (8 mL) at 0° C. Stir reaction and allow to warm to RT overnight. Dilute with EtOAc, wash sequentially with water, saturated aqueous sodium bicarbonate and brine. Dry over anhydrous magnesium sulfate, filter, and concentrate. Purify by flash chromatography (EtOAc/Hexanes) to yield tert-butyl N-[2-(6,7-dichloro-3-iodo-1H-indol-2-yl) ethyl] carbamate (1.25 g, 2.75 mmol, 72%). ES-MS (m/z): 398.6/400.6 (M+1, -tBu).

### Intermediate 20

### 2-(6,7-Dichloro-3-iodo-1H-indol-2-yl)ethanamine

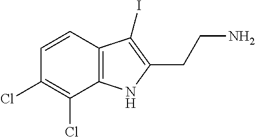 [SMILES: NCCc1[nH]c2c(Cl)c(Cl)ccc2c1I]

Add TFA (1 mL, 13.2 mmol) to tert-butyl N-[2-(6,7-dichloro-3-iodo-1H-indol-2-yl)ethyl]carbamate (1.26 g, 2.77 mmol) dissolved in DCM (10 mL) and anisole (3 mL, 27.5 mmol). Stir under nitrogen at RT. After 45 minutes, concentrate the reaction under reduced pressure and purify by SCX (rinse successively with CH2Cl2, 1:1 CH2Cl2:MeOH and MeOH; elute with 2 M NH₃-MeOH). Concentrate SCX eluent and resubject to same purification conditions to yield 2-(6,7-dichloro-3-iodo-1H-indol-2-yl) ethanamine (0.644 g, 1.84 mmol, 67%). ES-MS (m/z): 355.0/357.0 (M+1).

### Intermediate 21

### 2-Chloro-N-[2-(6,7-dichloro-3-iodo-1H-indol-2-yl)ethyl]acetamide

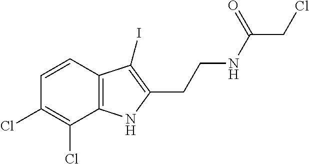 [SMILES: O=C(CCl)NCCc1[nH]c2c(Cl)c(Cl)ccc2c1I]

Dissolve 2-(6,7-dichloro-3-iodo-1H-indol-2-yl) ethanamine (0.59 g, 1.67 mmol) in DCM (16 mL) and add triethylamine (0.700 mL, 5.01 mmol). Add chloroacetyl chloride (0.200 mL, 2.52 mmol) in DCM 0° C. After 45 min, concentrate the reaction and purify by flash chromatography (EtOAc/Hexanes) to yield 2-chloro-N-[2-(6,7-dichloro-3-iodo-1H-indol-2-yl) ethyl] acetamide (0.30 g, 0.69 mmol, 41.5%). ES-MS (m/z): 431.0/433.0/435.0 (M+1).

### Intermediate 22

### 7,8-Dichloro-11-iodo-1,2,3,5-tetrahydro-[1,4]diazepino[1,7-a]indol-4-one

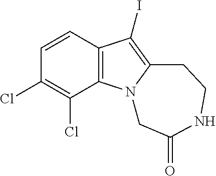 [SMILES: O=C1Cn2c(c(I)c3ccc(Cl)c(Cl)c32)CCN1]

In a microwave vial, dissolve 2-chloro-N-[2-(6,7-dichloro-3-iodo-1H-indol-2-yl)ethyl]acetamide (0.15 g, 0.35 mmol) in acetone (3.4 mL) and add cesium carbonate (0.11 g, 0.35 mmol)). Heat to 70° C. for 17 hrs. Dilute with EtOAc and filter through diatomaceous earth, rinsing with additional EtOAc. Concentrate to yield 7,8-dichloro-11-iodo-1,2,3,5-tetrahydro-[1,4]diazepino[1,7-a]indol-4-one (0.1 g, 0.24 mmol, 69.9%). ES-MS (m/z): 395.0/397.0 (M+1).

### Intermediate 23

### 7,8-Dichloro-11-(1-tetrahydropyran-2-ylpyrazol-4-yl)-1,2,3,5-tetrahydro-[1,4]diazepino[1,7-a]indol-4-one

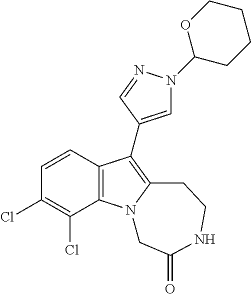 [SMILES: O=C1Cn2c(c(-c3cnn(C4CCCCO4)c3)c3ccc(Cl)c(Cl)c32)CCN1]

Add 7,8-dichloro-11-iodo-1,2,3,5-tetrahydro-[1,4]diazepino[1,7-a]indol-4-one (0.1 g, 0.24 mmol), 1-(tetrahydro-2H-pyran-2-yl)-4-(4,4,5,5-tetramethyl-1,3,2-dioxaborolan-2-yl)-1H-pyrazole (0.11 g, 0.36 mmol), potassium carbonate (0.10 g, 0.72 mmol), 1,4-dioxane (2 mL) and water (0.40 mL) to a microwave vial. Sparge with nitrogen. Add [1,1′-bis(diphenylphosphino) ferrocene]dichloropalladium(II) (0.02 g, 0.02 mmol) and heat to 80° C. for 3 hours. Filter the reaction through diatomaceous earth, rinsing with EtOAc. Purify by flash chromatography (EtOAc/Hexane/MeOH) yield 7,8-dichloro-11-(1-tetrahydropyran-2-ylpyrazol-4-yl)-1,2,3,5-tetrahydro-[1,4]diazepino[1,7-a]indol-4-one (0.08 g, 0.13 mmol, 53.6%). ES-MS (m/z): 335.0/337.0 (M+1, -THP).

### EXAMPLE 7

### 7,8-Dichloro-11-(1H-pyrazol-4-yl)-1,2,3,5-tetrahydro-[1,4]diazepino[1,7-a]indol-4-one

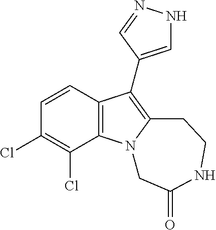 [SMILES: O=C1Cn2c(c(-c3cn[nH]c3)c3ccc(Cl)c(Cl)c32)CCN1]

Add hydrochloric acid (4.0 N in dioxane, 0.47 mL) to a solution of 7,8-dichloro-11-(1-tetrahydropyran-2-ylpyrazol-4-yl)-1,2,3,5-tetrahydro-[1,4]diazepino[1,7-a]indol-4-one (0.78 mg, 0.18 mmol) in 1,4-dioxane (2.4 mL) and methanol (0.58 mL) at 0° C. Allow to warm to RT. After 20 min, concentrate and redissolve in THF/water (4 mL, 1:1). Add potassium carbonate (130 mg, 0.93 mmol) and stir at 50° C. for 30 minutes. Concentrate and purify by HPLC. Yields 7,8-dichloro-11-(1H-pyrazol-4-yl)-1,2,3,5-tetrahydro-[1,4] diazepino[1,7-a]indol-4-one (0.02 mg, 0.047 mmol, 26%). ES-MS (m/z): 335.0/337.0 (M+1). ¹H NMR (400 MHz, DMSO): 7.86-7.84 (m, 3H), 7.55 (d, J=8.5 Hz, 1H), 7.29 (d, J=8.5 Hz, 1H), 5.50 (s, 2H), 3.46-3.41 (m, 2H), 3.36-3.30 (m, 2H).

### SCHEME FOR EXAMPLE 8

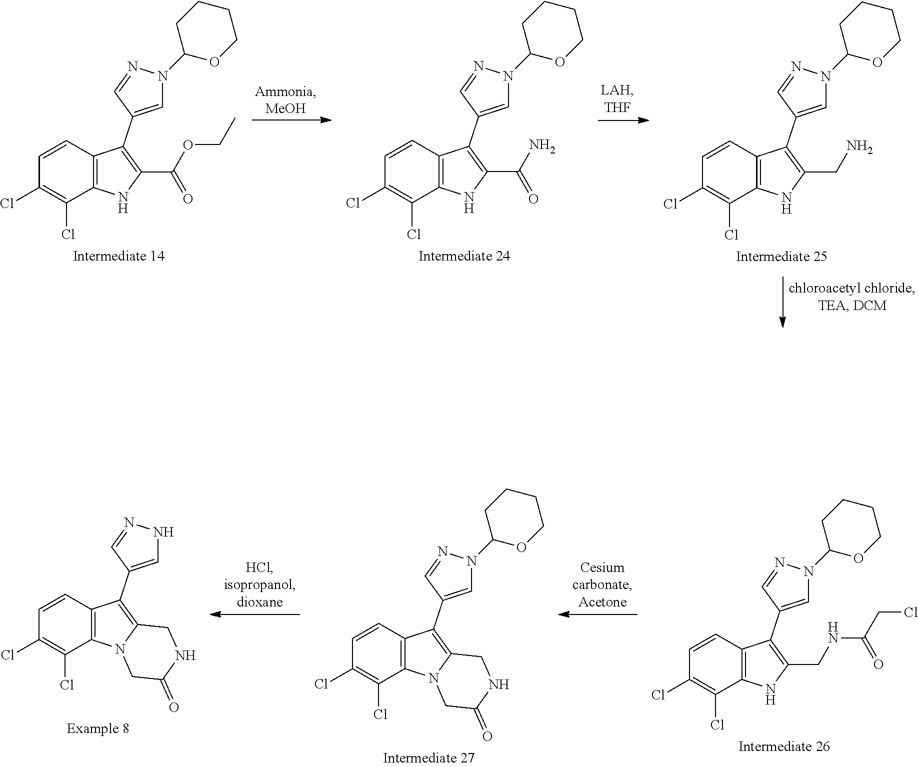

### Intermediate 24

### 6,7-Dichloro-3-(1-tetrahydropyran-2-ylpyrazol-4-yl)-1H-indole-2-carboxamide

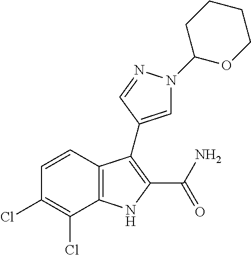 [SMILES: NC(=O)c1[nH]c2c(Cl)c(Cl)ccc2c1-c1cnn(C2CCCCO2)c1]

Dissolve ethyl 6,7-dichloro-3-(1-tetrahydropyran-2-ylpyrazol-4-yl)-1H-indole-2-carboxylate (0.68 mg, 1.65 mmol) in ammonia/methanol (10 mL, 2 M) and heat to 50° C. in a pressure vial for 2 days. After 48 hours, cool to RT and collect the solid by suction filtration, rinsing with DCM. Yields 6,7-dichloro-3-(1-tetrahydropyran-2-ylpyrazol-4-yl)-1H-indole-2-carboxamide (0.31 g, 0.80 mmol, 48.7%). ES-MS (m/z): 379.0/381.0 (M+1).

### Intermediate 25

### [6,7-Dichloro-3-(1-tetrahydropyran-2-ylpyrazol-4-yl)-1H-indol-2-yl]methanamine

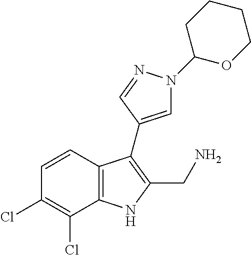 [SMILES: NCc1[nH]c2c(Cl)c(Cl)ccc2c1-c1cnn(C2CCCCO2)c1]

Add lithium aluminum hydride (0.070 g, 1.84 mmol) to a solution of 6,7-dichloro-3-(1 tetrahydropyran-2-ylpyrazol-4-yl)-1H-indole-2-carboxamide (0.31 g, 0.72 mmol) in THF (14 mL). Heat the reaction to 80° C. under N₂ for 3 hours. Monitor the reaction by LCMS and add more lithium aluminum hydride if needed to drive the reaction to completion. Cool to 0° C. and add water (1 ml), 2N sodium hydroxide (5 mL) and EtOAc. Stir at RT for 1 hour, decant the ethyl acetate layer. Extract with an additional portion of EtOAc and combine the organic layers. Dry over anhydrous magnesium sulfate, filter and concentrate to yield [6,7-dichloro-3-(1-tetrahydropyran-2-ylpyrazol-4-yl)-1H-indol-2-yl]methanamine (0.27 g, 0.59 mmol, 81.7%). ES-MS (m/z): 365.2/367.2 (M+1).

### Intermediate 26

### 2-Chloro-N-[[6,7-dichloro-3-(1-tetrahydropyran-2-ylpyrazol-4-yl)-1H-indol-2-yl]methyl]acetamide

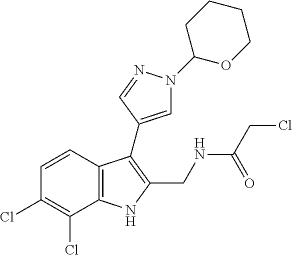 [SMILES: O=C(CCl)NCc1[nH]c2c(Cl)c(Cl)ccc2c1-c1cnn(C2CCCCO2)c1]

Dissolve [6,7-dichloro-3-(1-tetrahydropyran-2-ylpyrazol-4-yl)-1H-indol-2-yl]methanamine (0.27 g, 0.74 mmol) in DCM (7 mL) and add triethylamine (0.31 mL, 2.22 mmol). Add chloroacetyl chloride (0.088 mL, 1.10 mmol) as a solution in minimal DCM at 0° C. After 20 min, add water (0.2 mL) and concentrate. Purify by flash chromatography (EtOAc/Hexanes) to yield 2-chloro-N-[[6,7-dichloro-3-(1-tetrahydropyran-2-ylpyrazol-4-yl)-1H-indol-2-yl]methyl]acetamide (0.30 g, 0.67 mmol, 91.8%). ES-MS (m/z): 441.2/443.2.

### Intermediate 27

### 6,7-Dichloro-10-(1-tetrahydropyran-2-ylpyrazol-4-yl)-2,4-dihydro-1H-pyrazino[1,2-a]indol-3-one

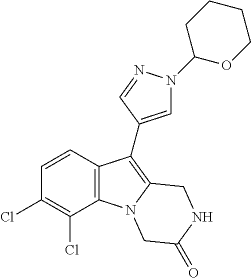 [SMILES: O=C1Cn2c(c(-c3cnn(C4CCCCO4)c3)c3ccc(Cl)c(Cl)c32)CN1]

Dissolve 2-chloro-N-[2-(6,7-dichloro-3-iodo-1H-indol-2-yl)ethyl]acetamide (0.13 g, 0.29 mmol) in acetone (3.0 mL) in a microwave vial and add cesium carbonate (0.09 g, 0.29 mmol). Heat to 70° C. for 20 min. Dilute with EtOAc, filter through diatomaceous earth rinsing with EtOAc/methanol and concentrate. Triturate with diethyl ether and decant. Dry the solid to yield 6,7-dichloro-10-(1-tetrahydropyran-2-ylpyrazol-4-yl)-2,4-dihydro-1H-pyrazino[1,2-a]indol-3-one (0.12 g, 0.30 mmol, 100%). ES-MS (m/z): 321.0/323.0 (M+1, -THP).

### EXAMPLE 8

### 6,7-Dichloro-10-(1H-pyrazol-4-yl)-2,4-dihydro-1H-pyrazino[1,2-a]indol-3-one

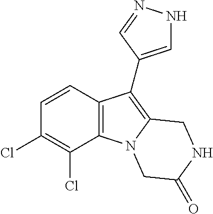 [SMILES: O=C1Cn2c(c(-c3cn[nH]c3)c3ccc(Cl)c(Cl)c32)CN1]

Add hydrochloric acid (4.0 N in dioxane, 0.51 mL) to a solution of 6,7-dichloro-10-(1-tetrahydropyran-2-ylpyrazol-4-yl)-2,4-dihydro-1H-pyrazino[1,2-a]indol-3-one (83 mg, 0.20 mmol) in 1,4-dioxane (2.58 mL) and IPA (0.64 mL) at 0° C. Remove ice bath and stir 90 min. Concentrate and purify by HPLC. Yields 6,7-dichloro-10-(1H-pyrazol-4-yl)-2,4-dihydro-1H-pyrazino[1,2-a]indol-3-one (0.01 mg, 0.03 mmol, 15.2%). ES-MS (m/z): 321.0/323.0 (M+1). ¹H NMR (400 MHz, DMSO): 7.91 (s, 2H), 7.67 (d, J=8.5 Hz, 1H), 7.30 (d, J=8.6 Hz, 1H), 5.23 (s, 2H), 4.66 (s, 2H), 3.33-3.30 (m, 16H), 2.52-2.50 (m, 35H), 2.00 (s, 2H), 1.24-1.16 (m, 2H).

### SCHEME FOR EXAMPLE 10

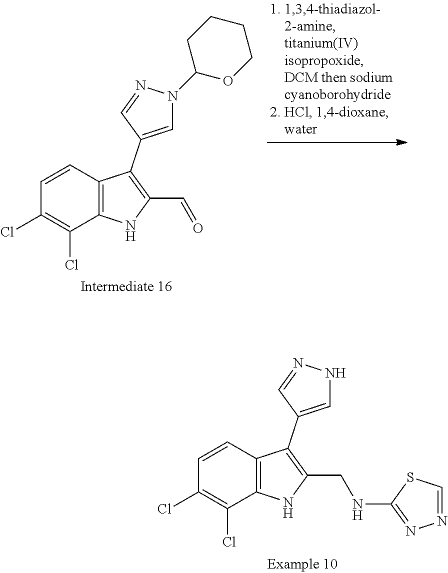 [SMILES: O=Cc1[nH]c2c(Cl)c(Cl)ccc2c1-c1cnn(C2CCCCO2)c1 ; Clc1ccc2c(-c3cn[nH]c3)c(CNc3nncs3)[nH]c2c1Cl]

### EXAMPLE 10

### N-[[6,7-Dichloro-3-(1H-pyrazol-4-yl)-1H-indol-2-yl]methyl]-1,3,4-thiadiazol-2-amine

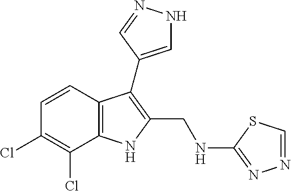 [SMILES: Clc1ccc2c(-c3cn[nH]c3)c(CNc3nncs3)[nH]c2c1Cl]

Dissolve 6,7-dichloro-3-(1-tetrahydropyran-2-ylpyrazol-4-yl)-1H-indole-2-carbaldehyde (100 mg, 0.275 mmol) in 1,2-dichloroethane (2 mL). Add 2-amino-1,3,4-thiadiazole (83 mg, 0.825 mmol) followed by titanium(IV) isopropoxide (0.240 mL, 0.81 mmol). Heat to 80° C. under nitrogen for 2 hours to form the imine then allow the reaction to cool to RT. Add sodium cyanoborohydride (70 mg, 1.05 mmol) and heat to 50° C. for 16 hours. Cooled the reaction to RT. Quench the reaction with 2 mL of 5% aqueous citric acid and stir open to the air for 1 hour. Dilute with DCM (5 mL) and filter to remove salts, rinsing with DCM. Separate the layers of the filtrate, stir with diatomaceous earth, filter and concentrate to give N-[[6,7-dichloro-3-(1-tetrahydropyran-2-ylpyrazol-4-yl)-1H-indol-2-yl]methyl]-1,3,4-thiadiazol-2-amine (159.5 mg, 0.21 mmol). Dissolve N-[[6,7-dichloro-3-(1-tetrahydropyran-2-ylpyrazol-4-yl)-1H-indol-2-yl]methyl]-1,3,4-thiadiazol-2-amine (159.5 mg, 0.21 mmol) in 1,4-dioxane (2 mL). Add hydrochloric acid (36.5% in water, 0.13 mL, 2 mmol) and stir at RT for 16 hours. Concentrate the reaction under a stream of nitrogen and purify by HPLC to yield N-[[6,7-dichloro-3-(1H-pyrazol-4-yl)-1H-indol-2-yl]methyl]-1,3,4-thiadiazol-2-amine (37.5 mg, 36% yield). ES-MS: 365.0/367.0 (M+1). ¹H NMR (400 MHz, DMSO): 13.09-13.04 (m, 1H), 11.74 (s, 1H), 8.69 (s, 1H), 8.09 (t, J=4.5 Hz, 1H), 8.02-7.99 (m, 1H), 7.78-7.73 (m, 1H), 7.63 (d, J=8.6 Hz, 1H), 7.24 (d, J=8.6 Hz, 1H), 4.69 (d, J=4.6 Hz, 2H).

### SCHEME FOR EXAMPLE 11

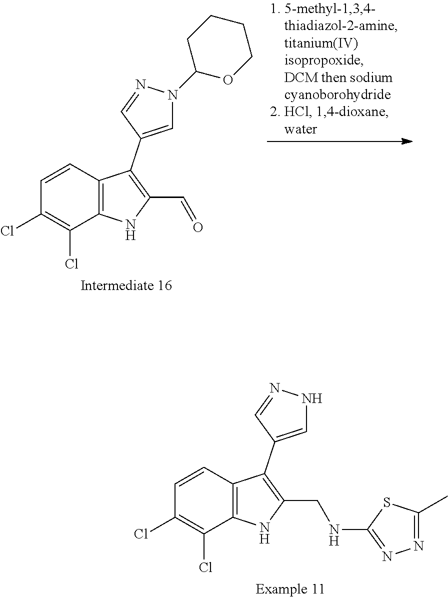 [SMILES: O=Cc1[nH]c2c(Cl)c(Cl)ccc2c1-c1cnn(C2CCCCO2)c1 ; Cc1nnc(NCc2[nH]c3c(Cl)c(Cl)ccc3c2-c2cn[nH]c2)s1]

### EXAMPLE 11

### N-[[6,7-Dichloro-3-(1H-pyrazol-4-yl)-1H-indol-2-yl]methyl]-5-methyl-1,3,4-thiadiazol-2-amine

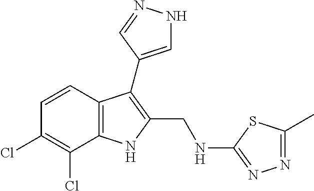 [SMILES: Cc1nnc(NCc2[nH]c3c(Cl)c(Cl)ccc3c2-c2cn[nH]c2)s1]

Dissolve 6,7-dichloro-3-(1-tetrahydropyran-2-ylpyrazol-4-yl)-1H-indole-2-carbaldehyde (100 mg, 0.275 mmol) in 1,2-dichloroethane (2 mL). Add 2-amino-5-methyl-1,3,4-thiadiazole (95 mg, 0.825 mmol) followed by titanium(IV) isopropoxide (0.240 mL, 0.81 mmol). Heat to 80° C. under nitrogen for 2 hours to form the imine then allow the reaction to return to RT. Add sodium cyanoborohydride (70 mg, 1.05 mmol) and heat to 50° C. for 16 hours. Cool the reaction to RT. Quench the reaction with 2 mL of 5% aqueous citric acid and stir open to the air for 1 hour. Dilute with DCM (5 mL) and filter to remove salts, rinsing with DCM. Separate the layers of the filtrate, stir the organic layer with diatomaceous earth, filter and concentrate to give N-[[6,7-dichloro-3-(1-tetrahydropyran-2-ylpyrazol-4-yl)-1H-indol-2-yl]methyl]-5-methyl-1,3,4-thiadiazol-2-amine (144 mg, 53%). Dissolve N-[[6,7-dichloro-3-(1-tetrahydropyran-2-ylpyrazol-4-yl)-1H-indol-2-yl]methyl]-5-methyl-1,3,4-thiadiazol-2-amine (144 mg, 0.14 mmol) in 1,4-dioxane (2 mL). Add hydrochloric acid (36.5% in water, 0.13 mL, 2 mmol) and stir at RT for 16 hours. Concentrate the reaction under a stream of nitrogen and purify by HPLC to yield N-[[6,7-dichloro-3-(1H-pyrazol-4-yl)-1H-indol-2-yl]methyl]-5-methyl-1,3,4-thiadiazol-2-amine (29 mg, 50% yield). ES-MS: 379.0/381.0 (M+1). ¹H NMR (400 MHz, DMSO): 13.06-12.99 (m, 1H), 11.71 (s, 1H), 8.01 (s, 1H), 7.89 (t, J=4.8 Hz, 1H), 7.77-7.75 (m, 1H), 7.63 (d, J=8.6 Hz, 1H), 7.24 (d, J=8.3 Hz, 1H), 4.63 (d, J=4.9 Hz, 2H).

### SCHEME FOR EXAMPLE 12

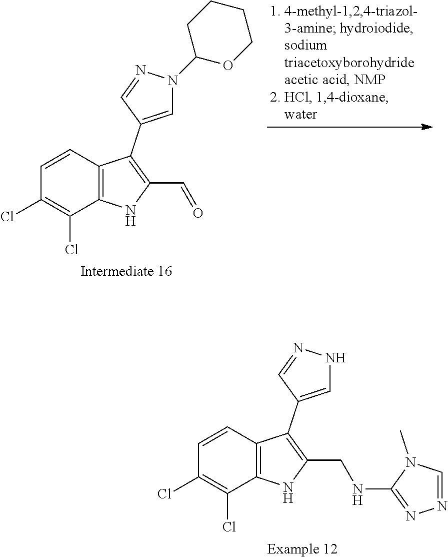 [SMILES: O=Cc1[nH]c2c(Cl)c(Cl)ccc2c1-c1cnn(C2CCCCO2)c1 ; Cn1cnnc1NCc1[nH]c2c(Cl)c(Cl)ccc2c1-c1cn[nH]c1]

### EXAMPLE 12

### N-[[6,7-Dichloro-3-(1H-pyrazol-4-yl)-1H-indol-2-yl]methyl]-4-methyl-1,2,4-triazol-3-amine

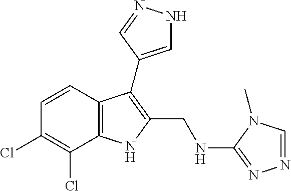 [SMILES: Cn1cnnc1NCc1[nH]c2c(Cl)c(Cl)ccc2c1-c1cn[nH]c1]

Dissolve 6,7-dichloro-3-(1-tetrahydropyran-2-ylpyrazol-4-yl)-1H-indole-2-carbaldehyde (150 mg, 0.411 mmol), 4-methyl-1,2,4-triazol-3-amine hydroiodide (278 mg, 1.23 mmol) and sodium triacetoxyborohydride (350 mg, 1.65 mmol) in 1-methyl-2-pyrrolidinone (2 mL). Add acetic acid (0.143 mL, 2.5 mmol). Stir at RT for 24 hours. Dilute with EtOAc (3 mL) and water (2 mL). Separate layers then extract the aqueous layer twice with EtOAc. Combine the organic extracts, stir with diatomaceous earth, filter and concentrate to give N-[[6,7-dichloro-3-(1-tetrahydropyran-2-ylpyrazol-4-yl)-1H-indol-2-yl]methyl]-4-methyl-1,2,4-triazol-3-amine hydroiodide (281 mg, 0.172 mmol). Dissolve this crude material in 1,4-dioxane (1.5 mL). Add hydrochloric acid (36.5% in water, 0.13 mL, 2 mmol) and stir at RT for 16 hours. Concentrate the reaction under a stream of nitrogen then purify by HPLC to yield N-[[6,7-dichloro-3-(1H-pyrazol-4-yl)-1H-indol-2-yl]methyl]-4-methyl-1,2,4-triazol-3-amine (25 mg, 17% yield). ES-MS: 362.2/364.2 (M+1). 1H NMR (400 MHz, DMSO): 13.08-12.99 (m, 1H), 11.63 (s, 1H), 8.10 (s, 1H), 8.02 (s, 1H), 7.83 (s, 1H), 7.60 (d, J=8.6 Hz, 1H), 7.23 (d, J=8.6 Hz, 1H), 6.66-6.61 (m, 1H), 4.59 (d, J=5.6 Hz, 2H), 3.39 (s, 3H).

### SCHEME FOR EXAMPLES 13 & 14

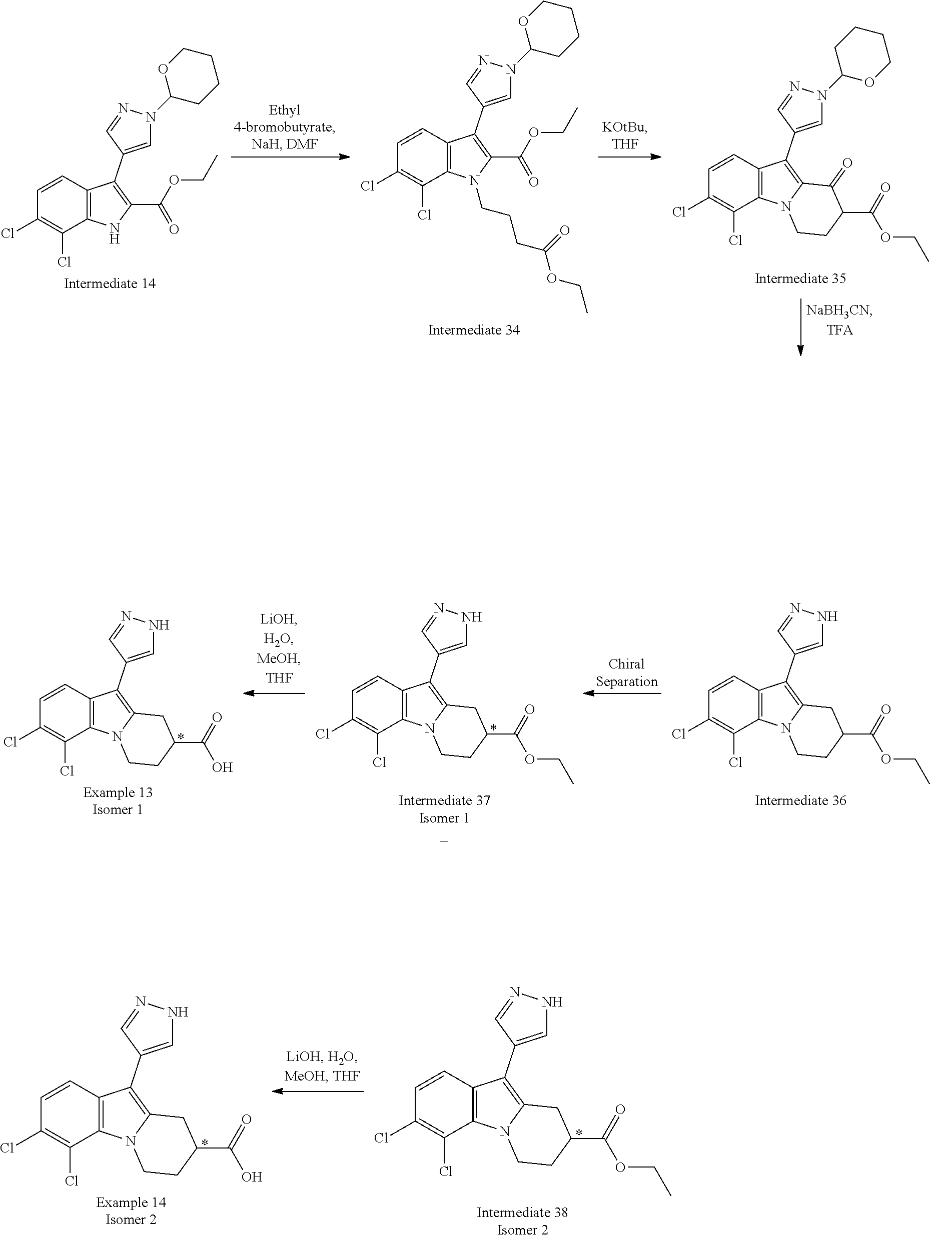

### Intermediate 34

### Ethyl 6,7-dichloro-1-(4-ethoxy-4-oxo-butyl)-3-(1-tetrahydropyran-2-ylpyrazol-4-yl)indole-2-carboxylate

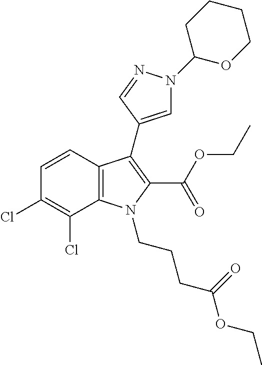 [SMILES: CCOC(=O)CCCn1c(C(=O)OCC)c(-c2cnn(C3CCCCO3)c2)c2ccc(Cl)c(Cl)c21]

Add sodium hydride (60%) in mineral oil (0.35 g, 8.7 mmol) portion wise to ethyl 6,7-dichloro-3-(1-tetrahydropyran-2-ylpyrazol-4-yl)-1H-indole-2-carboxylate (3.0 g, 7.1 mmol) in DMF (50 mL) while stirring, under nitrogen at RT. After 20 mins, add ethyl-4-bromobutyrate (1.5 mL, 10.8 mmol) and heat to 80° C. for 3 hours. Cool to RT, pour into water and extract with EtOAc (3×). Wash the combined organics with water and brine (2×), dry over anhydrous magnesium sulfate, filter and concentrate under reduced pressure. Purify by flash chromatography (EtOAc/hexanes) to yield ethyl 6,7-dichloro-1-(4-ethoxy-4-oxo-butyl)-3-(1-tetrahydropyran-2-ylpyrazol-4-yl)indole-2-carboxylate (3.9 g, 7.2 mmol 102%, purity>85%). ES-MS (m/z): 522.2/524.2 (M+H).

### Intermediate 35

### Ethyl 3,4-dichloro-9-oxo-10-(1-tetrahydropyran-2-ylpyrazol-4-yl)-7,8-dihydro-6H-pyrido[1,2-a] indole-8-carboxylate

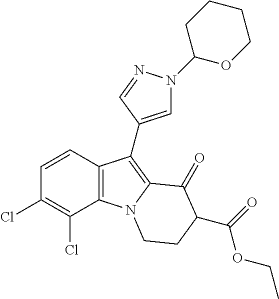 [SMILES: CCOC(=O)C1CCn2c(c(-c3cnn(C4CCCCO4)c3)c3ccc(Cl)c(Cl)c32)C1=O]

Add potassium tert-butoxide (0.76 g, 6.7 mmol) in THF (5 mL) to 6,7-dichloro-1-(4-ethoxy-4-oxo-butyl)-3-(1-tetrahydropyran-2-ylpyrazol-4-yl)indole-2-carboxylate (2.4 g, 4.5 mmol) in THF (10 mL) with stirring, under nitrogen at RT. Stir for 1.5 hrs. Pour into water and extract with EtOAc (3×). Wash the combined organics with water and brine, dry over anhydrous magnesium sulfate, filter, and concentrate. Purify by flash chromatography (EtOAc/DCM) to yield ethyl 3,4-dichloro-9-oxo-10-(1-tetrahydropyran-2-ylpyrazol-4-yl)-7,8-dihydro-6H-pyrido[1,2-a]indole-8-carboxylate (1.6 g, 3.2 mmol, 72% yield). ES-MS (m/z): 476.2/478.2 (M+1).

### Intermediate 36

### Ethyl 3,4-dichloro-10-(1H-pyrazol-4-yl)-6,7,8,9-tetrahydropyrido[1,2-a]indole-8-carboxylate

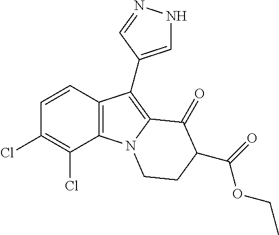 [SMILES: CCOC(=O)C1CCn2c(c(-c3cn[nH]c3)c3ccc(Cl)c(Cl)c32)C1=O]

Add sodium cyanoborohydride (1.5 g, 23 mmol) portion wise to ethyl 3,4-dichloro-9-oxo-10-(1-tetrahydropyran-2-yl] pyrazol-4-yl)-7,8-dihydro-6H-pyrido[1,2-a]indole-8-carboxylate (2.4 g, 4.9 mmol) in TFA (20 mL) with stirring, under nitrogen at RT. Stir for 1 hour and concentrate. Redissolve in DCM, carefully pour into water and extract with DCM (3×). Wash the combined organics with brine, dry over anhydrous magnesium sulfate, filter, and concentrate. Purify by HPLC to yield ethyl-3,4-dichloro-10-(1H-pyrazol-4-yl)-6,7,8,9-tetrahydropyrido[1,2-a]indole-8-carboxylate (0.58 g, 1.5 mmol, 31%). ES-MS (m/z): 378.0/380.0 (M+1).

### Intermediates 37 & 38

### Ethyl 3,4-dichloro-10-(1H-pyrazol-4-yl)-6,7,8,9-tetrahydropyrido[1,2-a]indole-8-carboxylate Isomer 1 and Isomer 2

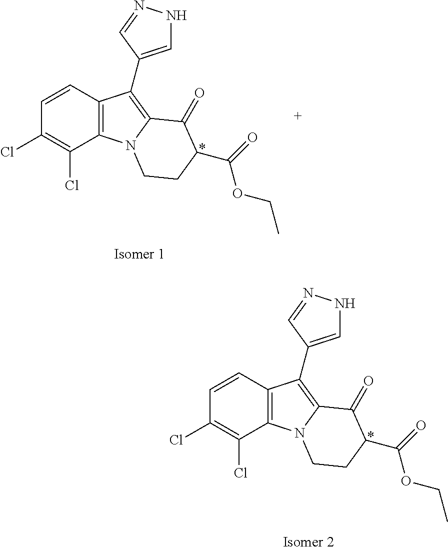 [SMILES: CCOC(=O)C1CCn2c(c(-c3cn[nH]c3)c3ccc(Cl)c(Cl)c32)C1=O ; CCOC(=O)C1CCn2c(c(-c3cn[nH]c3)c3ccc(Cl)c(Cl)c32)C1=O]

Purify ethyl-3,4-dichloro-10-(1H-pyrazol-4-yl)-6,7,8,9-tetrahydropyrido[1,2-a]indole-8-carboxylate (0.26 g, 0.67 mmol) by supercritical fluid chromatography to yield the titled compounds (Isomer 1-63 mg, 0.16 mmol, 24%; Isomer 2-67 mg, 0.17 mmol, 26%). Rₜ=1.58 (99% ee) and 1.85 (97% ee) minutes. Column: Chiralpak AD-H, 21×150 mm; Mobile Phase: 40% MeOH (w/0.5% DMEA): 60% CO₂; Flow Rate: 80 mL/min; Column temperature: 40° C.; Detection: 241 nM. ES-MS (m/z): 377.8/379.8 (M+1).

### EXAMPLE 13

### 3,4-Dichloro-10-(1H-pyrazol-4-yl)-6,7,8,9-tetrahydropyrido[1,2-a]indole-8-carboxylic acid Isomer 1

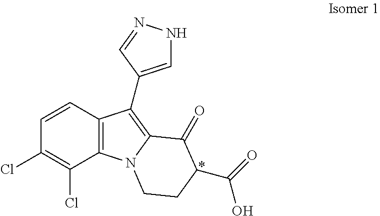 [SMILES: O=C(O)C1CCn2c(c(-c3cn[nH]c3)c3ccc(Cl)c(Cl)c32)C1=O]

Add lithium hydroxide (0.02 g, 0.84 mmol) to ethyl-3,4-dichloro-10-(1H-pyrazol-4-yl)-6,7,8,9-tetrahydropyrido[1,2-a]indole-8-carboxylate (Isomer 1-0.06 g, 0.15 mmol) in THF (1 mL), MeOH (0.66 mL) and water (0.33 mL) with stirring at RT. Heat to 50° C. for 1 hour. Cool to RT, neutralize via dropwise addition of 5 N HCl and concentrate under a stream of nitrogen gas. Triturate from acetonitrile/water, collect by suction filtration and dry in a vacuum oven to yield 3,4-dichloro-10-(1H-pyrazol-4-yl)-6,7,8,9-tetrahydropyrido[1,2-a]indole-8-carboxylic acid (Isomer 11—0.035 g, 0.1 mmol, 65%). ES-MS (m/z): 349.6/351.6 (M+1). ¹H NMR (400 MHz, DMSO): 13.12-13.09 (m, 1H), 7.85 (s, 2H), 7.56 (d, J=8.5 Hz, 1H), 7.23 (d, J=8.5 Hz, 1H), 4.94 (dt, J=11.9, 4.9 Hz, 1H), 4.47-4.40 (m, 1H), 3.42-3.40 (m, 2H), 2.87-2.81 (m, 1H), 2.39-2.33 (m, 1H), 2.14-2.06 (m, 1H).

### EXAMPLE 14

### 3,4-Dichloro-10-(1H-pyrazol-4-yl)-6,7,8,9-tetrahydropyrido[1,2-a]indole-8-carboxylic acid Isomer 2

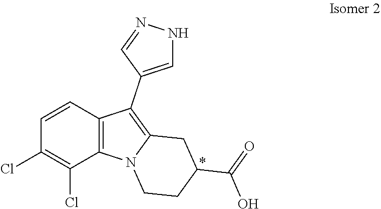 [SMILES: O=C(O)C1CCn2c(c(-c3cn[nH]c3)c3ccc(Cl)c(Cl)c32)C1]

Add lithium hydroxide (0.02 g, 0.84 mmol) to ethyl-3,4-dichloro-10-(1H-pyrazol-4-yl)-6,7,8,9-tetrahydropyrido[1,2-a]indole-8-carboxylate (Isomer 22—0.065 g, 0.17 mmol) in THF (1 mL), MeOH (0.66 mL) and water (0.33 mL) with stirring at RT. Heat to 50° C. for 1 hour. Cool to RT, neutralize via dropwise addition of 5 N HCl and concentrate under a stream of nitrogen gas. Triturate from acetonitrile/water, collect by suction filtration and dry in a vacuum oven to yield 3,4-dichloro-10-(1H-pyrazol-4-yl)-6,7,8,9-tetrahydropyrido[1,2-a]indole-8-carboxylic acid (Isomer 2-0.043 g, 0.1 mmol, 71%). ES-MS (m/z): 350.0/352.0 (M+1). ¹H NMR (400 MHz, DMSO): 13.12-13.09 (m, 1H), 7.85 (s, 2H), 7.56 (d, J=8.5 Hz, 1H), 7.23 (d, J=8.5 Hz, 1H), 4.94 (dt, J=11.9, 4.9 Hz, 1H), 4.47-4.40 (m, 1H), 3.42-3.40 (m, 2H), 2.87-2.81 (m, 1H), 2.39-2.33 (m, 1H), 2.14-2.06 (m, 1H).

### SCHEME FOR EXAMPLES 15 & 16

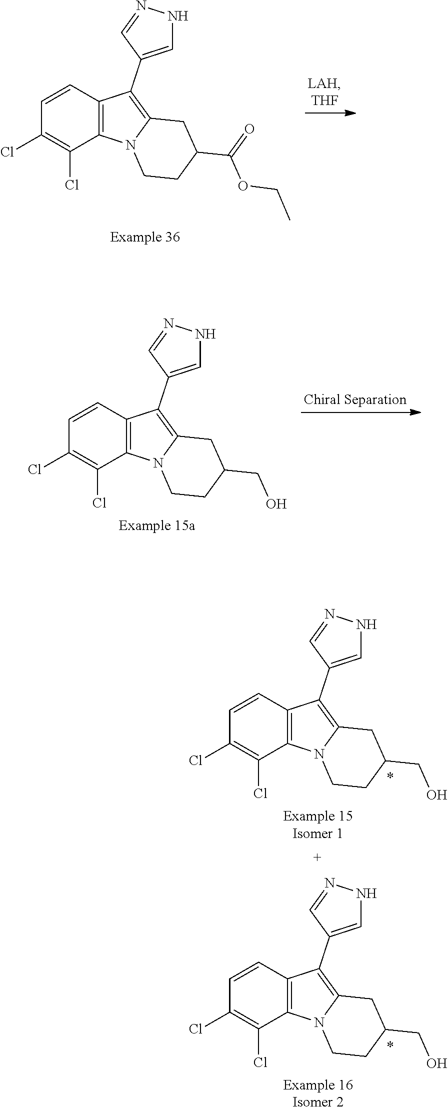 [SMILES: CCOC(=O)C1CCn2c(c(-c3cn[nH]c3)c3ccc(Cl)c(Cl)c32)C1 ; OCC1CCn2c(c(-c3cn[nH]c3)c3ccc(Cl)c(Cl)c32)C1 ; OCC1CCn2c(c(-c3cn[nH]c3)c3ccc(Cl)c(Cl)c32)C1 ; OCC1CCn2c(c(-c3cn[nH]c3)c3ccc(Cl)c(Cl)c32)C1]

### EXAMPLE 15A

### [3,4-Dichloro-10-(1H-pyrazol-4-yl)-6,7,8,9-tetrahydropyrido[1,2-a]indol-8-yl]methanol

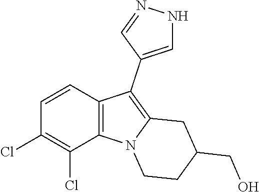 [SMILES: OCC1CCn2c(c(-c3cn[nH]c3)c3ccc(Cl)c(Cl)c32)C1]

Add lithium aluminum hydride (2.0 mol/L) in THF (0.3 mL, 0.6 mmol) to ethyl 3,4-dichloro-10-(1H-pyrazol-4-yl)-6,7,8,9-tetrahydropyrido[1,2-a]indole-8-carboxylate (0.2 g, 0.51 mmol) in THF (3 mL) with stirring, under nitrogen at −78° C. Gradually warm to 0° C. After 1 hour at 0° C., quench by dropwise addition of 20 mL of water, 20 mL of 15% aqueous NaOH and 60 mL of water successively with stirring at 0° C. Dilute with THF and warm to RT with vigorous stirring. Add anhydrous magnesium sulfate to the stirred solution. Filter through diatomaceous earth and concentrate. Purify by HPLC to yield [3,4-dichloro-10-(1H-pyrazol-4-yl)-6,7,8,9-tetrahydropyrido[1,2-a]indol-8-yl]methanol (0.051 g, 0.15 mmol, 29%). ES-MS (m/z): 335.6/337.6 (M+1).

### EXAMPLES 15 & 16

### [3,4-Dichloro-10-(1H-pyrazol-4-yl)-6,7,8,9-tetrahydropyrido[1,2-a]indol-8-yl]methanol Isomer 1 and Isomer 2

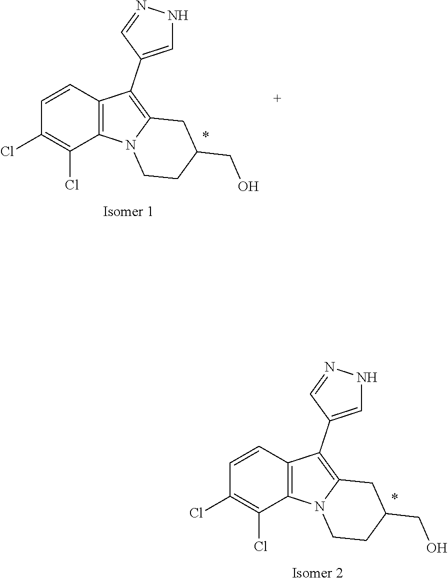 [SMILES: OCC1CCn2c(c(-c3cn[nH]c3)c3ccc(Cl)c(Cl)c32)C1 ; OCC1CCn2c(c(-c3cn[nH]c3)c3ccc(Cl)c(Cl)c32)C1]

Purify [3,4-dichloro-10-(1H-pyrazol-4-yl)-6,7,8,9-tetrahydropyrido[1,2-a]indol-8-yl]methanol (0.050 g, 0.14 mmol) by supercritical fluid chromatography to yield the titled compounds (Isomer 1-20 mg, 0.057 mmol, 40%; Isomer 2-13 mg, 0.038 mmol, 27%). Rₜ=2.68 (93% ee) and 3.01 (98% ee) minutes. Column: Chiralcel OD-H, 21×250 mm; Mobile Phase: 30% MeOH (w/0.5% DMEA): 70% CO₂; Flow Rate: 80 mL/min; Column temperature: 40° C.; Detection: 244 nM. ES-MS (m/z): 335.6/337.6 (M+1). ¹H NMR (400 MHz, DMSO): 13.05-13.00 (m, 1H), 7.99-7.92 (m, 2H), 7.56 (d, J=8.5 Hz, 1H), 7.22 (d, J=8.5 Hz, 1H), 5.04 (ddd, J=12.3, 5.4, 3.0 Hz, 1H), 4.73 (t, J=5.3 Hz, 1H), 4.33-4.26 (m, 1H), 3.50-3.43 (m, 2H), 3.18-3.05 (m, 1H) 2.67-2.63 (m, 1H), 2.19-2.15 (m, 1H), 1.96-1.93 (m, 1H), 1.75-1.64 (m, 1H).

### SCHEME FOR EXAMPLE 17

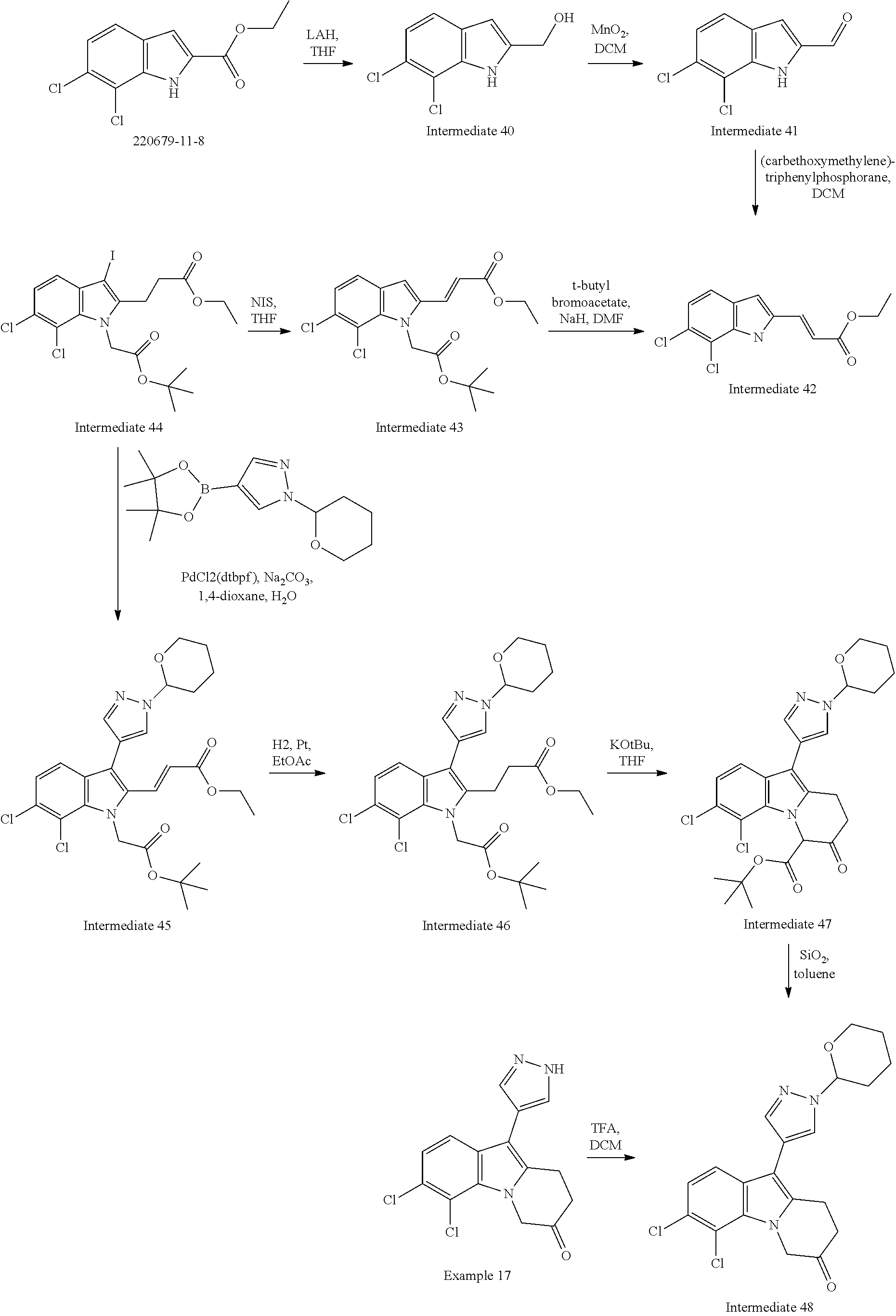

### Intermediate 40

### (6,7-Dichloro-1H-indol-2-yl)methanol

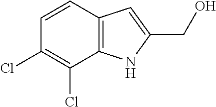 [SMILES: OCc1cc2ccc(Cl)c(Cl)c2[nH]1]

Add lithium aluminum hydride (2.0 mol/L) in THF (2.25 mL, 4.5 mmol) dropwise to ethyl 6,7-dichloro-1H-indole-2-carboxylate (1.0 g, 3.76 mmol) in THF (12 mL) with stirring, under nitrogen at 0° C. Gradually warm to RT and stir 16 hours. Dilute with diethyl ether and carefully quench with dropwise addition of 170 mL of water, 170 mL of 15% aqueous NaOH and 500 mL of water successively. Stir vigorously for 20 min. Add anhydrous magnesium sulfate to the stirred solution. Filter through diatomaceous earth and concentrate to yield (6,7-dichloro-1H-indol-2-yl)methanol (0.85 g, 3.8 mmol, 101%, purity>85%). ES-MS (m/z): 216.0/218.0 (M+1).

### Intermediate 41

### 6,7-Dichloro-1H-indole-2-carbaldehyde

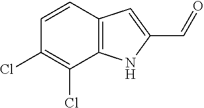 [SMILES: O=Cc1cc2ccc(Cl)c(Cl)c2[nH]1]

Add manganese dioxide (3.0 g, 34.5 mmol) to (6,7-dichloro-1H-indol-2-yl)methanol (0.847 g, 3.8 mmol) in DCM (15 mL) with stirring, under nitrogen at RT. Stir for 5 hours. Filter through diatomaceous earth and concentrate to a solution of 6,7-dichloro-1H-indole-2-carboxaldehyde in less than 20 mL of DCM that was carried forward without further purification. ES-MS (m/z): 211.4/213.4 (M−1, negative ionization mode) with purity>70%.

### Intermediate 42

### Ethyl (E)-3-(6,7-dichloro-1H-indol-2-yl)prop-2-enoate

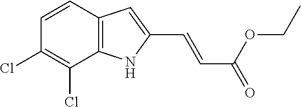 [SMILES: CCOC(=O)/C=C/c1cc2ccc(Cl)c(Cl)c2[nH]1]

Add (carbethoxymethylene)triphenylphosphorane (1.3 g, 3.8 mmol) to 6,7-dichloro-1H-indole-2-carboxaldehyde (0.84 g, 3.8 mmol) in DCM (15 mL) with stirring, under nitrogen at RT. Heat to reflux for 16 hours. Cool to RT, concentrate, and purify by flash chromatography (EtOAc/hexanes) to yield ethyl-3-(6,7-dichloro-1H-indol-2-yl)prop-2-enoate (0.51 g, 1.75 mmol, 46%). ES-MS (m/z): 284.0/286.0 (M+1).

### Intermediate 43

### Ethyl (E)-3-[1-(2-tert-butoxy-2-oxo-ethyl)-6,7-dichloro-indol-2-yl]prop-2-enoate

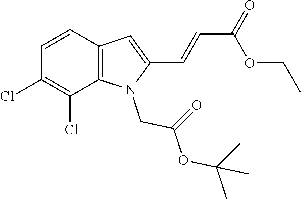 [SMILES: CCOC(=O)/C=C/c1cc2ccc(Cl)c(Cl)c2n1CC(=O)OC(C)(C)C]

Add sodium hydride (60 mass %) in mineral oil (0.085 g, 2.1 mmol) to ethyl-3-(6,7-dichloro-1H-indol-2-yl)prop-2-enoate (0.51 g, 1.7 mmol) in DMF (8 mL) with stirring, under nitrogen at RT. Stir 20 min and add tert-butyl bromoacetate (0.35 mL, 2.3 mmol). Stir for 3 hours. Pour into water and extract with EtOAc (3×). Wash the combined organics with water and brine. Dry over anhydrous magnesium sulfate, filter, and concentrate. Purify by flash chromatography (EtOAc/hexanes) to yield ethyl-3-[1-(2-tert-butoxy-2-oxo-ethyl)-6,7-dichloro-indol-2-yl]prop-2-enoate (0.68 g, 1.65 mmol, 95%). ES-MS (m/z): 397.8/399.8 (M+1).

### Intermediate 44

### Ethyl (E)-3-[1-(2-tert-butoxy-2-oxo-ethyl)-6,7-dichloro-3-iodo-indol-2-yl]prop-2-enoate

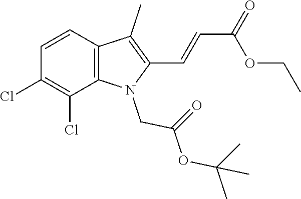 [SMILES: CCOC(=O)/C=C/c1c(C)c2ccc(Cl)c(Cl)c2n1CC(=O)OC(C)(C)C]

Add N-iodosuccinimide (0.335 g, 1.5 mmol) in DMF (1 mL) to ethyl-3-[1-(2-tert-butoxy-2-oxo-ethyl)-6,7-dichloro-indol-2-yl]prop-2-enoate (0.5 g, 1.2 mmol) in DMF (5 mL) with stirring, under nitrogen at RT. Stir for 16 hours. Pour into saturated aqueous sodium thiosulfate and extract with EtOAc (3×). Wash the combined organics with water and brine. Dry over anhydrous magnesium sulfate, filter, and concentrate. Purify by flash chromatography (EtOAc/hexanes) to yield ethyl-3-[1-(2-tert-butoxy-2-oxo-ethyl)-6,7-dichloro-3-iodo-indol-2-yl]prop-2-enoate (0.58 g, 1.08 mmol, 89%). ES-MS (m/z): 524.0/526.0 (M+1).

### Intermediate 45

### Ethyl (E)-3-[1-(2-tert-butoxy-2-oxo-ethyl)-6,7-dichloro-3-(1-tetrahydropyran-2-ylpyrazol-4-yl)indol-2-yl]prop-2-enoate

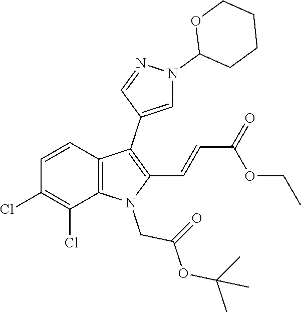 [SMILES: CCOC(=O)/C=C/c1c(-c2cnn(C3CCCCO3)c2)c2ccc(Cl)c(Cl)c2n1CC(=O)OC(C)(C)C]

Add 1,1′-Bis(di-tert-butylphosphino)ferrocene-palladium dichloride (0.14 g, 0.21 mmol) to a stirred solution of 1-(tetrahydro-2H-pyran-2-yl)-4-(4,4,5,5-tetramethyl-1,3,2-dioxaborolan-2-yl)-1H-pyrazole (0.615 g, 2.2 mmol), ethyl-3-[1-(2-tert-butoxy-2-oxo-ethyl)-6,7-dichloro-3-iodo-indol-2-yl]prop-2-enoate (0.585 g, 1.1 mmol) and sodium carbonate (0.34 g, 3.2 mmol) in 1,4-dioxane (6 mL) and water (1.5 mL) at RT. Degas with a stream of nitrogen gas for 10 mins. and heat to 90° C. for 2 hours. Cool to RT, pour into water and extract with EtOAc (3×). Wash the combined organics with brine, dry over anhydrous magnesium sulfate, filter, and concentrate. Purify by flash chromatography (EtOAc/hexanes) to yield ethyl-3-[1-(2-tert-butoxy-2-oxo-ethyl)-6,7-dichloro-3-(1-tetrahydropyran-2-ylpyrazol-4-yl)indol-2-yl]prop-2-enoate (0.42 g, 0.75 mmol, 69%). ES-MS (m/z): 547.9/549.9 (M+1).

### Intermediate 46

### Ethyl 3-[1-(2-tert-butoxy-2-oxo-ethyl)-6,7-dichloro-3-(1-tetrahydropyran-2-ylpyrazol-4-yl)indol-2-yl]propanoate

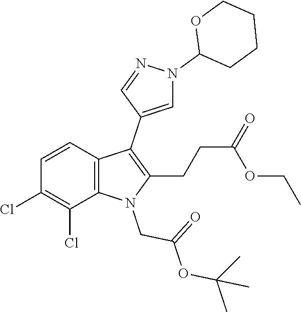 [SMILES: CCOC(=O)CCc1c(-c2cnn(C3CCCCO3)c2)c2ccc(Cl)c(Cl)c2n1CC(=O)OC(C)(C)C]

Add platinum (5 wt. %) on carbon (0.05 g, 0.25 mmol) to a pressure vessel with EtOAc (10 mL) under nitrogen at RT. Add Ethyl-3-[1-(2-tert-butoxy-2-oxo-ethyl)-6,7-dichloro-3-(1-tetrahydropyran-2-ylpyrazol-4-yl)indol-2-yl]prop-2-enoate (0.42 g, 0.74 mmol) in EtOAc (10 mL) and seal vessel. Purge with nitrogen and then pressurize with hydrogen gas to 60 PSI. Shake at RT for 24 hrs. Depressurize and purge with nitrogen. Filter through diatomaceous earth and concentrate. Purify by flash chromatography (EtOAc/hexanes) to yield ethyl -3-[1-(2-tert-butoxy -2-oxo-ethyl)-6,7-dichloro-3-(1-tetrahydropyran-2-ylpyrazol-4-yl)indol-2-yl]propanoate (0.38 g, 0.68 mmol, 92%). ES-MS (m/z): 550.0/552.0 (M+1).

### Intermediate 47

### tert-Butyl 3,4-dichloro-7-oxo-10-(1-tetrahydropyran-2-ylpyrazol -4-yl)-8,9-dihydro-6H-pyrido[1,2-a]indole-6-carboxylate

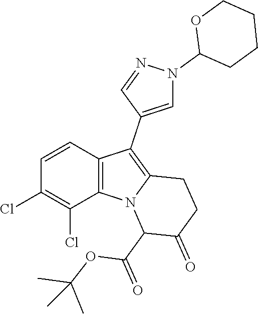 [SMILES: CC(C)(C)OC(=O)C1C(=O)CCc2c(-c3cnn(C4CCCCO4)c3)c3ccc(Cl)c(Cl)c3n21]

Add potassium tert-butoxide (0.12 g, 1.1 mmol) to ethyl-3-[1-(2-tert-butoxy-2-oxo-ethyl)-6,7-dichloro-3-(1-tetrahydropyran-2-ylpyrazol-4-yl)indol-2-yl]propanoate (0.385 g, 0.68 mmol) in THF (3 mL), with stirring, under nitrogen at RT. Stir for 2 hours. Pour into water and extract with EtOAc (3×). Wash the combined organic extracts with brine, dry over anhydrous magnesium sulfate, filter and concentrate to yield tert-butyl 3,4-dichloro-7-oxo-10-(1-tetrahydropyran-2-ylpyrazol-4-yl)-8,9-dihydro-6H-pyrido[1,2-a]indole-6-carboxylate (0.27 g, 0.77 mmol, 77%). ES-MS (m/z): 504.2/506.2 (M+1).

### Intermediate 48

### 3,4-Dichloro-10-(1-tetrahydropyran-2-ylpyrazol-4-yl)-8,9-dihydro-6H-pyrido[1,2-a]indol-7-one

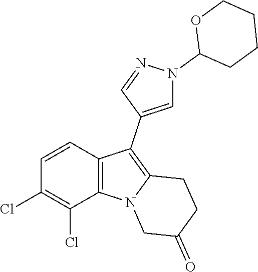 [SMILES: O=C1CCc2c(-c3cnn(C4CCCCO4)c3)c3ccc(Cl)c(Cl)c3n2C1]

Add silica gel (0.2 g, 3.3 mmol) to tert-butyl 3,4-dichloro-7-oxo-10-(1-tetrahydropyran-2-ylpyrazol-4-yl)-8,9-dihydro-6H-pyrido[1,2-a]indole-6-carboxylate (0.27 g, 0.52 mmol) in toluene (5 mL) with stirring, under nitrogen at RT. Heat to reflux for 3 hours. Filter and concentrate. Purify by flash chromatography (EtOAc/DCM) to yield 3,4-dichloro-10-(1-tetrahydropyran-2-ylpyrazol-4-yl)-8,9-dihydro-6H-pyrido[1,2-a]indol-7-one (0.14 g, 0.62 mmol, 62%). ES-MS (m/z): 404.0/406.0 (M+1).

### EXAMPLE 17

### 3,4-Dichloro-10-(1H-pyrazol-4-yl)-8,9-dihydro-6H-pyrido[1,2-a]indol-7-one

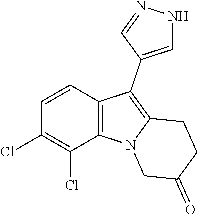 [SMILES: O=C1CCc2c(-c3cn[nH]c3)c3ccc(Cl)c(Cl)c3n2C1]

Treat 3,4-dichloro-10-(1-tetrahydropyran-2-ylpyrazol-4-yl)-8,9-dihydro-6H-pyrido[1,2-a]indol-7-one (50 mg, 0.11 mmol) in DCM (1.5 mL) with TFA (0.5 mL) and stir at RT for 2 hours. Concentrate, dilute with saturated aqueous sodium bicarbonate and extract with DCM (2×). Dry the combined organic layers with anhydrous sodium sulfate, filter, and concentrate. Purify by flash chromatography (EtOAc/petroleum ether) to yield 3,4-dichloro-10-(1H-pyrazol-4-yl)-8,9-dihydro-6H-pyrido[1,2-a]indol-7-one (32 mg, 0.099 mmol, 84%). ES-MS (m/z): 319.8/321.8 (M+1). ¹H NMR (400 MHz, DMSO): 7.91 (s, 2H), 7.61 (d, J=8.5 Hz, 1H), 7.27 (d, J=8.5 Hz, 1H), 5.30 (s, 2H), 3.29-3.25 (m, 2H), 2.73-2.69 (m, 2H).

### SCHEME FOR EXAMPLES 18 & 19

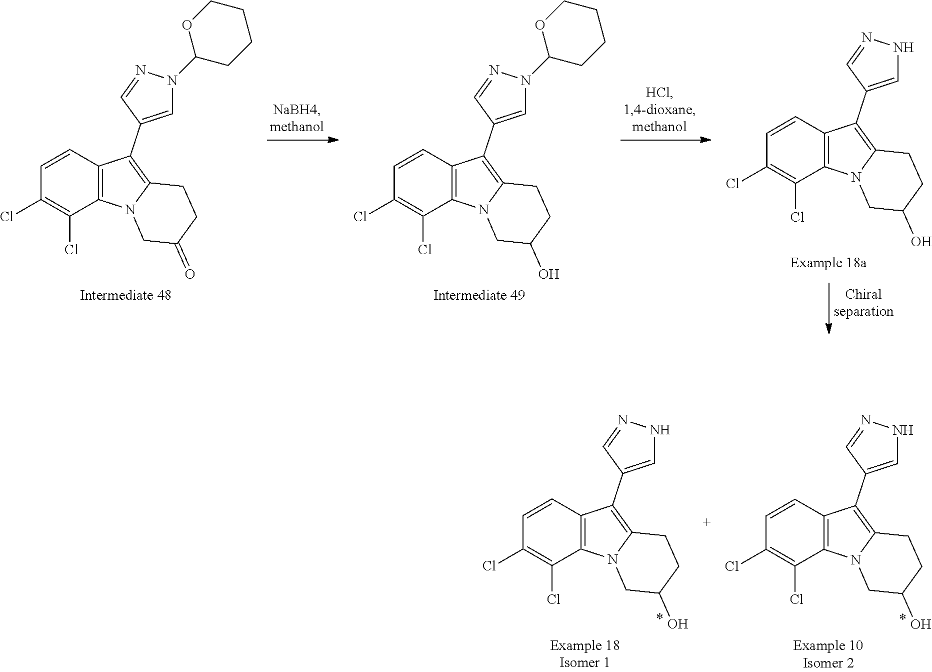 [SMILES: O=C1CCc2c(-c3cnn(C4CCCCO4)c3)c3ccc(Cl)c(Cl)c3n2C1 ; OC1CCc2c(-c3cnn(C4CCCCO4)c3)c3ccc(Cl)c(Cl)c3n2C1 ; OC1CCc2c(-c3cn[nH]c3)c3ccc(Cl)c(Cl)c3n2C1 ; OC1CCc2c(-c3cn[nH]c3)c3ccc(Cl)c(Cl)c3n2C1 ; OC1CCc2c(-c3cn[nH]c3)c3ccc(Cl)c(Cl)c3n2C1]

### Intermediate 49

### 3,4-Dichloro-10-(1-tetrahydropyran-2-ylpyrazol-4-yl)-6,7,8,9-tetrahydropyrido[1,2-a]indol-7-ol

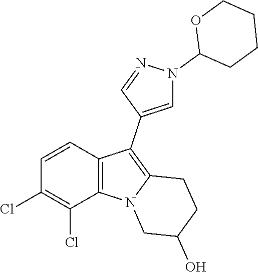 [SMILES: OC1CCc2c(-c3cnn(C4CCCCO4)c3)c3ccc(Cl)c(Cl)c3n2C1]

Add sodium borohydride (0.027 g, 0.71 mmol) to 3,4-dichloro-10-(1-tetrahydropyran-2-ylpyrazol-4-yl)-8,9-dihydro-6H-pyrido[1,2-a]indol-7-one (0.25 g, 0.6 mmol) in MeOH (4 mL) with stirring, under nitrogen at RT. Stir for 1 hour. Pour into water and extract with EtOAc (3×). Dry the combined organics over anhydrous magnesium sulfate, filter, and concentrate to yield 3,4-dichloro-10-(1-tetrahydropyran-2-ylpyrazol-4-yl)-6,7,8,9-tetrahydropyrido[1,2-a]indol-7-ol (0.250 g, 0.59 mmol, 99%). ES-MS (m/z): 406.0/408.0 (M+1).

### EXAMPLE 18A

### 3,4-Dichloro-10-(1H-pyrazol-4-yl)-6,7,8,9-tetrahydropyrido[1,2-a]indol-7-ol

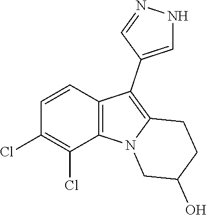 [SMILES: OC1CCc2c(-c3cn[nH]c3)c3ccc(Cl)c(Cl)c3n2C1]

Add hydrochloric acid (4.0 mol/L) in dioxane (1.5 mL, 6.0 mmol) to 3,4-dichloro-10-(1-tetrahydropyran-2-ylpyrazol-4-yl)-6,7,8,9-tetrahydropyrido[1,2-a]indol-7-ol (0.25 g, 0.59 mmol) in 1,4-dioxane (6 mL) and MeOH (2 mL) with stirring, under nitrogen at RT. The reaction was allowed to stir at RT for 2 hours. The reaction was concentrated to dryness via a stream of nitrogen gas. The material was purified via reverse phase flash chromatography (acetonitrile/water/0.1% formic acid) to yield 3,4-dichloro-10-(1H-pyrazol-4-yl)-6,7,8,9-tetrahydropyrido[1,2-a]indol-7-ol (0.14 g, 0.42 mmol, 71%). ES-MS (m/z): 321.6/323.6 (M+1).

### EXAMPLES 18 & 19

### 3,4-Dichloro-10-(1H-pyrazol-4-yl)-6,7,8,9-tetrahydropyrido[1,2-a]indol-7-ol Isomer 1 and Isomer 2

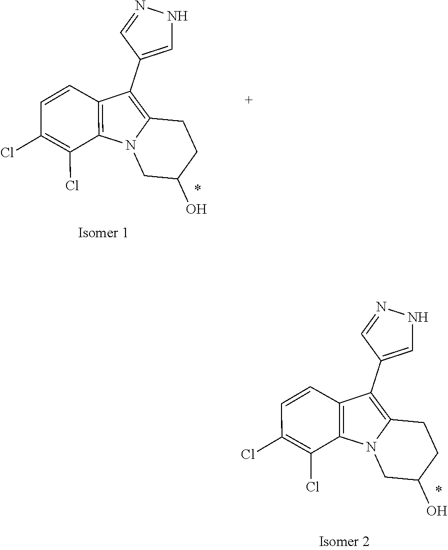 [SMILES: OC1CCc2c(-c3cn[nH]c3)c3ccc(Cl)c(Cl)c3n2C1 ; OC1CCc2c(-c3cn[nH]c3)c3ccc(Cl)c(Cl)c3n2C1]

Purify 3,4-dichloro-10-(1H-pyrazol-4-yl)-6,7,8,9-tetrahydropyrido[1,2-a]indol-7-ol (0.14 g, 0.42 mmol) by supercritical fluid chromatography to yield the titled compounds (Isomer 1-64 mg, 0.046 mmol, 46%; Isomer 2-65 mg, 0.047 mmol, 46%). Rₜ=1.79 (99% ee) and 2.97 (98% ee) minutes. Column: (S,S) Whelk-01, 21×250 mm; Mobile Phase: 40% EtOH (w/0.5% DMEA): 60% CO₂; Flow Rate: 80 mL/min; Column temperature: 40° C.; Detection: 242 nM. ES-MS (m/z): 321.6/323.6 (M+1). ¹H NMR (400 MHz, DMSO): 13.16-13.13 (m, 1H), 8.09-8.06 (m, 2H), 7.57 (d, J=8.5 Hz, 1H), 7.22 (d, J=8.5 Hz, 1H), 5.33-5.25 (m, 1H), 4.70 (dd, J=4.2, 12.2 Hz, 1H), 4.51 (dd, J=5.2, 12.3 Hz, 1H), 4.24 (d, J=4.1 Hz, 1H), 3.17-3.09 (m, 1H), 2.94 (dt, J=17.1, 6.1 Hz, 1.97-1.92 (m, 2H).

### SCHEME FOR EXAMPLES 20 & 21

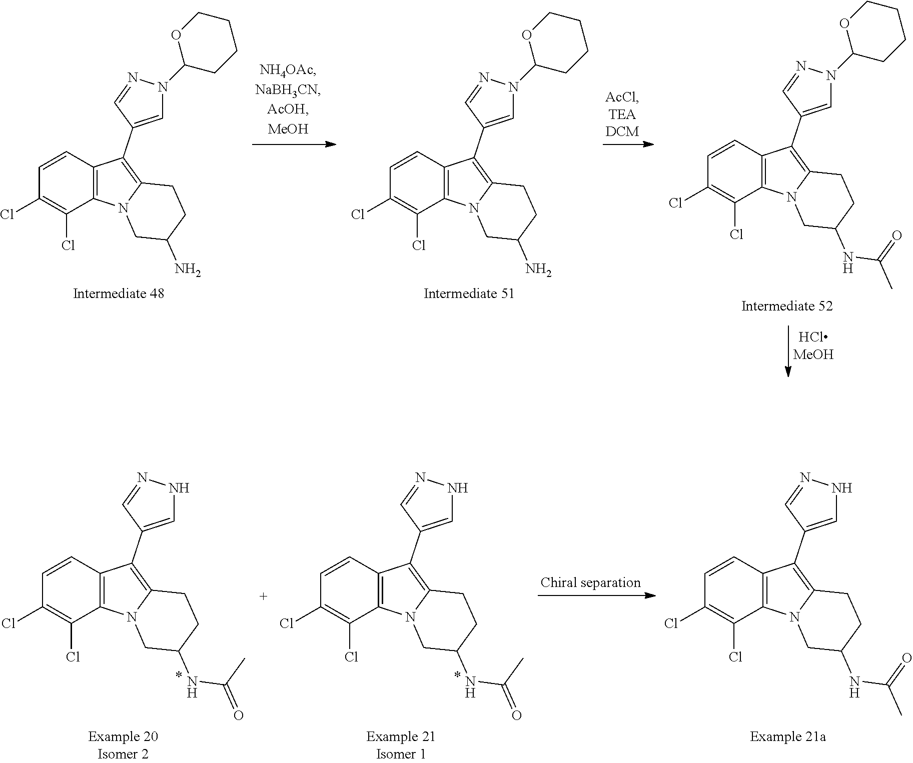 [SMILES: NC1CCc2c(-c3cnn(C4CCCCO4)c3)c3ccc(Cl)c(Cl)c3n2C1 ; NC1CCc2c(-c3cnn(C4CCCCO4)c3)c3ccc(Cl)c(Cl)c3n2C1 ; CC(=O)NC1CCc2c(-c3cnn(C4CCCCO4)c3)c3ccc(Cl)c(Cl)c3n2C1 ; CC(=O)NC1CCc2c(-c3cn[nH]c3)c3ccc(Cl)c(Cl)c3n2C1 ; CC(=O)NC1CCc2c(-c3cn[nH]c3)c3ccc(Cl)c(Cl)c3n2C1 ; CC(=O)NC1CCc2c(-c3cn[nH]c3)c3ccc(Cl)c(Cl)c3n2C1]

### Intermediate 51

### 3,4-Dichloro-10-(1-tetrahydropyran-2-ylpyrazol-4-yl)-6,7,8,9-tetrahydropyrido[1,2-a]indol-7-amine

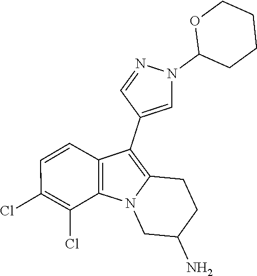 [SMILES: NC1CCc2c(-c3cnn(C4CCCCO4)c3)c3ccc(Cl)c(Cl)c3n2C1]

Add sodium cyanoborohydride (170 mg, 2.68 mmol) to 3,4-dichloro-10-(1-tetrahydropyran-2-ylpyrazol-4-yl)-8,9-dihydro-6H-pyrido[1,2-a]indol-7-one (600 mg, 1.34 mmol) ammonium acetate (1.54 g, 20.0 mmol) and acetic acid (804 mg, 13.4 mmol) in methanol (10 mL, 247 mmol) and stir for 2 hours. Neutralize with saturated aqueous sodium bicarbonate and extract with EtOAc (×2). Dry the combined organics over anhydrous sodium sulfate, filter, and concentrate. Purify by flash chromatography (MeOH/DCM) to yield 3,4-dichloro-10-(1-tetrahydropyran-2-ylpyrazol-4-yl)-6,7,8,9-tetrahydropyrido[1,2-a]indol-7-amine (300 mg, 0.633 mmol, 47% Yield, 85% purity).

### Intermediate 52

### N-[3,4-Dichloro-10-(1-tetrahydropyran-2-ylpyrazol-4-yl)-6,7,8,9-tetrahydropyrido[1,2-a]indol-7-yl]acetamide

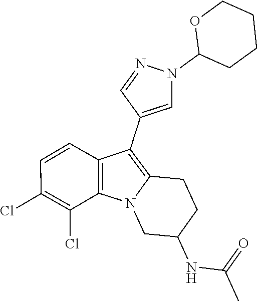 [SMILES: CC(=O)NC1CCc2c(-c3cnn(C4CCCCO4)c3)c3ccc(Cl)c(Cl)c3n2C1]

Dissolve 3,4-dichloro-10-(1-tetrahydropyran-2-ylpyrazol-4-yl)-6, 7, 8,9-tetrahydropyrido[1, 2-a] indol-7-amine (150 mg, 0.316 mmol) and triethylamine (0.17 mL, 1.20 mmol) in DCM (3 mL). Add acetyl chloride (0.04 mL, 0.600 mmol) at 0° C. Stir at 0° C. for 1 hour. Quench with saturated aqueous sodium bicarbonate and dilute with water, extract with ethyl acetate, dry over sodium sulfate, filter, and concentrate. Purify by flash column chromatography (EtOAc) to yield N-[3,4-dichloro-10-(1-tetrahydropyran-2-ylpyrazol-4-yl)-6,7,8,9-tetrahydropyrido[1,2-a]indol-7-yl]acetamide (85 mg, 0.16 mmol, 52% Yield) as a white solid.

### EXAMPLE 21A

### N-[3,4-Dichloro-10-(1H-pyrazol-4-yl)-6,7,8,9-tetrahydropyrido[1,2-a]indol-7-yl]acetamide

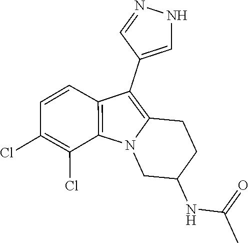 [SMILES: CC(=O)NC1CCc2c(-c3cn[nH]c3)c3ccc(Cl)c(Cl)c3n2C1]

Dissolve N-[3,4-dichloro-10-(1-tetrahydropyran-2-ylpyrazol-4-yl)-6,7,8,9-tetrahydropyrido[1,2-a]indol-7-yl]acetamide (85 mg, 0.17 mmol) in 4 N HCl/MeOH (3 mL). Stir at RT for 1 hour and concentrate. Purify by prep-HPLC to yield N-[3, 4-dichloro-10-(1H-pyrazol-4-yl)-6, 7, 8, 9-tetrahydropyrido[1, 2-a]indol-7-yl]acetamide (42 mg, 0.11 mmol, 69% Yield) as a white solid. ES-MS (m/z): 362.6/364.6 (M+1).

### EXAMPLES 20 & 21

### N-[3,4-Dichloro-10-(1H-pyrazol-4-yl)-6,7,8,9-tetrahydropyrido[1,2-a]indol-7-yl]acetamide Isomer 1 and Isomer 2

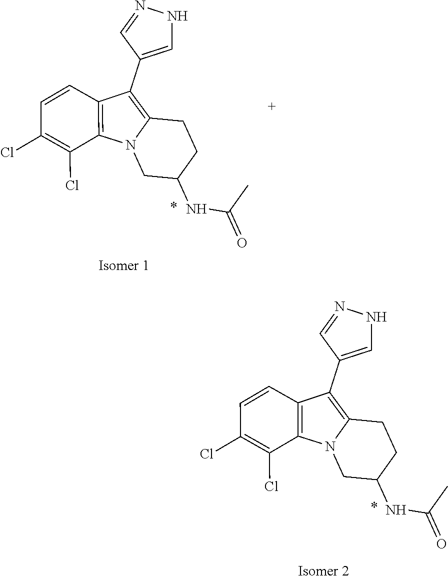 [SMILES: CC(=O)NC1CCc2c(-c3cn[nH]c3)c3ccc(Cl)c(Cl)c3n2C1 ; CC(=O)NC1CCc2c(-c3cn[nH]c3)c3ccc(Cl)c(Cl)c3n2C1]

Purify N-[3,4-dichloro-10-(1H-pyrazol-4-yl)-6,7,8,9-tetrahydropyrido[1,2-a]indol-7-yl]acetamide (0.039 g, 0.107 mmol) by supercritical fluid chromatography to yield the titled compounds (Isomer 1-16 mg, 0.044 mmol, 41%; Isomer 2-15 mg, 0.042 mmol, 39%). Rₜ=1.36 (95% ee) and 1.87 (95% ee) minutes. Column: Chiralpak AS-H, 21×150 mm; Mobile Phase: 25% MeOH: 75% CO₂; Flow Rate: 80 mL/min; Column temperature: 40° C.; Detection: 225 nM. ES-MS (m/z): 362.6/364.6 (M+1). 1H NMR (400 MHz, DMSO): 13.07-13.06 (m, 1H), 8.24-8.20 (m, 1H), 8.04-8.01 (m, 1H), 7.87-7.86 (m, 1H), 7.59 (d, J=8.5 Hz, 1H), 7.24 (d, J=8.5 Hz, 1H), 4.91-4.87 (m, 1H), 4.36-4.29 (m, 2H), 3.16-3.12 (m, 2H), 1.88 (s, 5H).

### SCHEMES FOR EXAMPLES 22 & 23

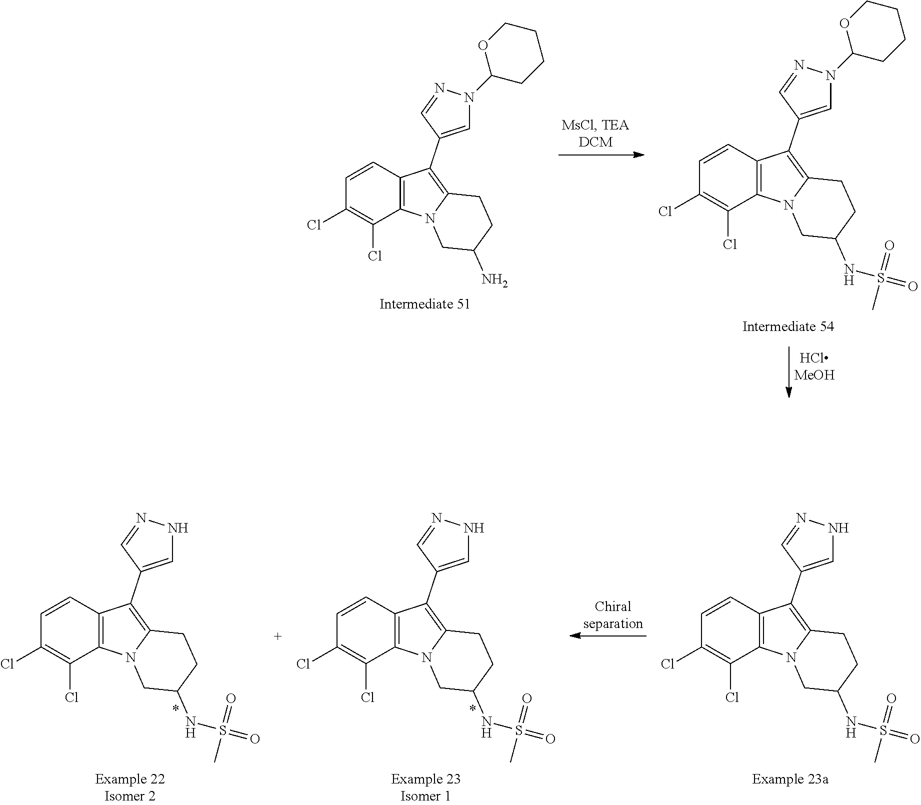 [SMILES: NC1CCc2c(-c3cnn(C4CCCCO4)c3)c3ccc(Cl)c(Cl)c3n2C1 ; CS(=O)(=O)NC1CCc2c(-c3cnn(C4CCCCO4)c3)c3ccc(Cl)c(Cl)c3n2C1 ; CS(=O)(=O)NC1CCc2c(-c3cn[nH]c3)c3ccc(Cl)c(Cl)c3n2C1 ; CS(=O)(=O)NC1CCc2c(-c3cn[nH]c3)c3ccc(Cl)c(Cl)c3n2C1 ; CS(=O)(=O)NC1CCc2c(-c3cn[nH]c3)c3ccc(Cl)c(Cl)c3n2C1]

### Intermediate 54

### N-[3,4-Dichloro-10-(1-tetrahydropyran-2-ylpyrazol-4-yl)-6,7,8,9-tetrahydropyrido[1,2-a]indol-7-yl]methanesulfonamide

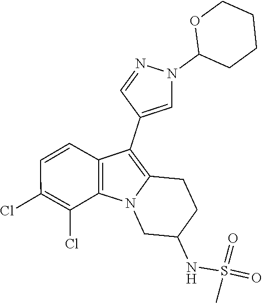 [SMILES: CS(=O)(=O)NC1CCc2c(-c3cnn(C4CCCCO4)c3)c3ccc(Cl)c(Cl)c3n2C1]

Dissolve 3, 4-dichloro-10-(1-tetrahydropyran-2-ylpyrazol-4-yl)-6, 7, 8, 9-tetrahydropyrido[1, 2-a] indol-7-amine (150 mg, 0.316 mmol) and triethylamine (0.17 mL, 1.2 mmol) in DCM (3 mL). Add methanesulfonyl chloride (140 mg, 1.21 mmol) at 0° C. Stir at RT for 1 hour. Quench with saturated aqueous sodium bicarbonate and dilute with water, extract with EtOAc, dry over sodium sulfate, filter, and concentrate. Purify by flash column chromatography (EtOAc/petroleum ether) to yield N-[3,4-dichloro-10-(1-tetrahydropyran-2-ylpyrazol-4-yl)-6,7,8,9-tetrahydropyrido[1,2-a]indol-7-yl]methanesulfonamide (100 mg, 0.184 mmol, 58% Yield) as a white solid.

### EXAMPLE 23A

### N-[3,4-Dichloro-10-(1H-pyrazol-4-yl)-6,7,8,9-tetrahydropyrido[1,2-a]indol-7-yl]methanesulfonamide

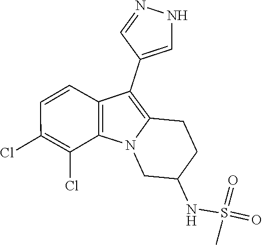 [SMILES: CS(=O)(=O)NC1CCc2c(-c3cn[nH]c3)c3ccc(Cl)c(Cl)c3n2C1]

Dissolve N-[3,4-dichloro-10-(1-tetrahydropyran-2-ylpyrazol-4-yl)-6,7,8,9-tetrahydropyrido[1,2-a]indol-7-yl]methanesulfonamide (100 mg, 0.184 mmol) in 4N HCl/MeOH (5 mL). Stir at RT for 1 hour and concentrate. Purify by prep-HPLC to yield N-[3,4-dichloro-10-(1H-pyrazol-4-yl)-6, 7, 8, 9-tetrahydropyrido[1,2-a]indol-7-yl]methanesulfonamide (41 mg, 0.10 mmol, 56% Yield).

### EXAMPLES 22 & 23

### N-[3,4-Dichloro-10-(1H-pyrazol-4-yl)-6,7,8,9-tetrahydropyrido[1,2-a]indol-7-yl]methanesulfonamide

### Isomer 1 and Isomer 2

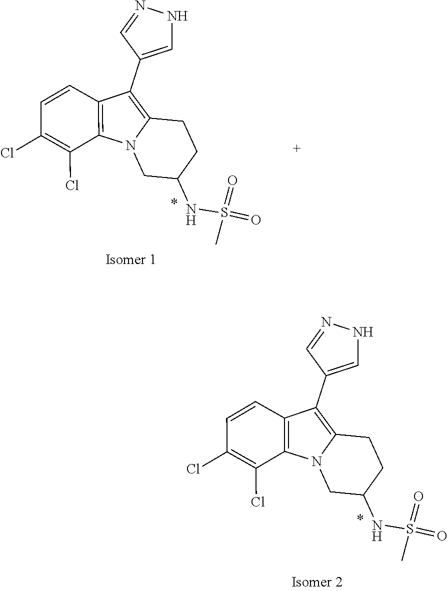 [SMILES: CS(=O)(=O)NC1CCc2c(-c3cn[nH]c3)c3ccc(Cl)c(Cl)c3n2C1 ; CS(=O)(=O)NC1CCc2c(-c3cn[nH]c3)c3ccc(Cl)c(Cl)c3n2C1]

Purify N-[3,4-dichloro-10-(1H-pyrazol-4-yl)-6,7,8,9-tetrahydropyrido[1,2-a]indol-7-yl]methanesulfonamide (0.039 g, 0.097 mmol) by supercritical fluid chromatography to yield the titled compounds (Isomer 1-14 mg, 0.037 mmol, 37%; Isomer 2-14 mg, 0.037 mmol, 37%). Rₜ=1.94 (99% ee) and 2.77 (97% ee) minutes; Column: Chiralcel OJ-H, 21×150 mm; Mobile Phase: 40% MeOH: 60% CO₂; Flow Rate: 80 mL/min; Column temperature: 40° C.; Detection: 225 nM. ES-MS (m/z): 396.4/398.4 (M−1, negative ionization mode). ¹H NMR (400.13 MHz, DMSO): 13.06-12.98 (m, 1H), 7.99-7.94 (m, 1H), 7.79-7.75 (m, 1H), 7.60-7.53 (m, 2H), 7.24 (d, J=8.4 Hz, 1H), 4.95 (dd, J=5.0, 12.1 Hz, 1H), 4.41 (dd, J=7.7, 12.2 Hz, 1H), 3.99-3.95 (m, 1H), 3.18-3.06 (m, 5H), 2.14-2.09 (m, 1H), 1.91-1.83 (m, 1H).

### SCHEME FOR EXAMPLES 24 & 25

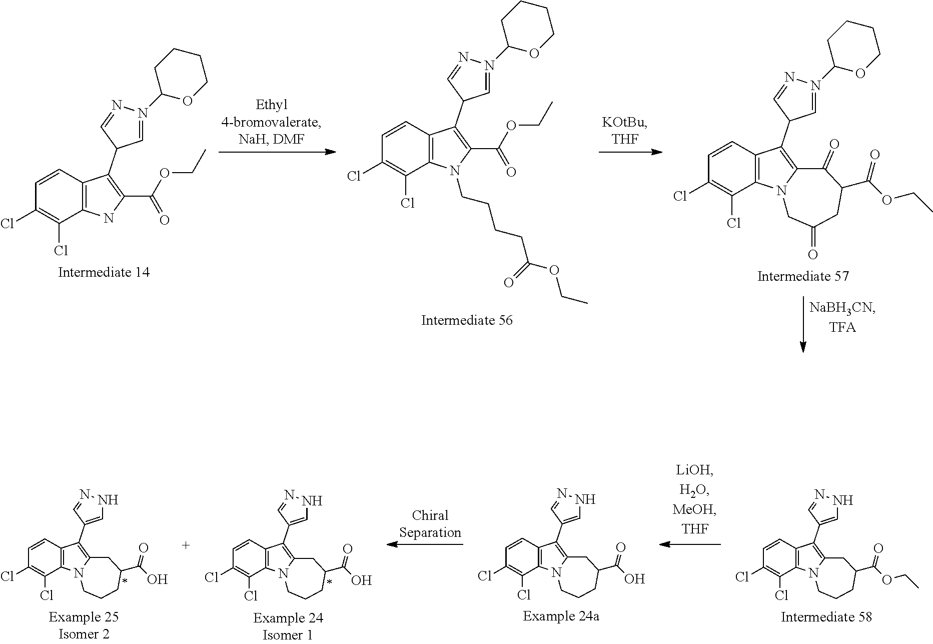

### Intermediate 56

### Ethyl 6,7-dichloro-1-(5-ethoxy-5-oxo-pentyl)-3-(1-tetrahydropyran-2-ylpyrazol-4-yl)indole-2-carboxylate

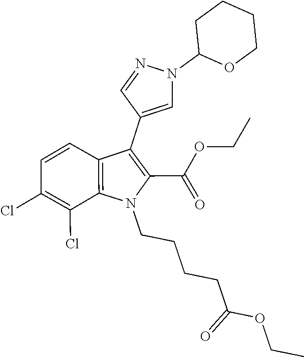 [SMILES: CCOC(=O)CCCCn1c(C(=O)OCC)c(-c2cnn(C3CCCCO3)c2)c2ccc(Cl)c(Cl)c21]

Add sodium hydride (60 mass %) in mineral oil (0.13 g, 3.25 mmol) ethyl 6,7-dichloro-3-(1-tetrahydropyran-2-ylpyrazol-4-yl)-1H-indole-2-carboxylate (1.1 g, 2.6 mmol) in DMF (15 mL) with stirring, under nitrogen at RT. Add ethyl 5-bromovalerate (0.62 mL, 3.9 mmol) after 20 min. Heat to 80° C. for 3 hours. Cool to RT, pour into water and extract with EtOAc (3×). Wash the combined organics with water and brine (2×), dry over anhydrous magnesium sulfate, filter, and concentrate. Purify by flash chromatography (EtOAc/hexanes) to yield ethyl 6,7-dichloro-1-(5-ethoxy-5-oxo-pentyl)-3-(1-tetrahydropyran-2-ylpyrazol-4-yl)indole-2-carboxylate (1.44 g, 2.6 mmol, 100%). ES-MS (m/z): 536.4/538.4 (M+1).

### Intermediate 57

### Ethyl 3,4-dichloro-10-oxo-11-(1-tetrahydropyran-2-ylpyrazol-4-yl)-6,7,8,9-tetrahydroazepino[1,2-a]indole-9-carboxylate

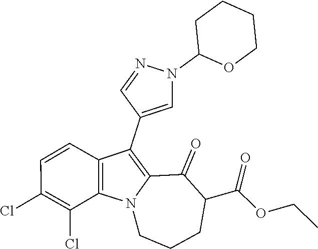 [SMILES: CCOC(=O)C1CCCn2c(c(-c3cnn(C4CCCCO4)c3)c3ccc(Cl)c(Cl)c32)C1=O]

Add potassium tert-butoxide (0.43 g, 3.8 mmol) to ethyl 6,7-dichloro-1-(5-ethoxy-5-oxo-pentyl)-3-(1-tetrahydropyran-2-ylpyrazol-4-yl)indole-2-carboxylate (1.4 g, 2.5 mmol) in THF (8 mL) with stirring, under nitrogen at 0° C. Allow to warm to RT and stir for 1-2 hrs. Pour into water and extract with EtOAc (3×). Wash the combined organics with brine, dry over anhydrous magnesium sulfate, filter, and concentrate. Purify by flash chromatography (EtOAc/hexanes) to yield ethyl 3,4-dichloro-10-oxo-11-(1-tetrahydropyran-2-ylpyrazol-4-yl)-6,7,8,9-tetrahydroazepino[1,2-a]indole-9-carboxylate (0.67 g, 1.3 mmol, 53%). ES-MS (m/z): 489.8/491.8 (M+1).

### Intermediate 58

### Ethyl 3,4-dichloro-11-(1H-pyrazol-4-yl)-7,8,9,10-tetrahydro-6H-azepino[1,2-a]indole-9-carboxylate

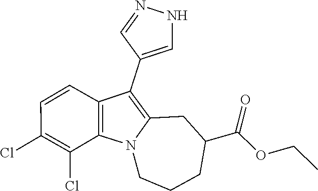 [SMILES: CCOC(=O)C1CCCn2c(c(-c3cn[nH]c3)c3ccc(Cl)c(Cl)c32)C1]

Add sodium cyanoborohydride (0.44 g, 6.6 mmol) portion wise to ethyl 3,4-dichloro-10-oxo-11-(1-tetrahydropyran-2-ylpyrazol-4-yl)-6,7,8,9-tetrahydroazepino[1,2-a]indole-9-carboxylate (0.67 g, 1.3 mmol) in TFA (10 mL) with stirring, under nitrogen at RT. Allow to warm to RT and stir for 1 hour. Concentrate and purify by reverse phase flash chromatography (acetonitrile/water/0.1% formic acid) to yield ethyl 3,4-dichloro-11-(1H-pyrazol-4-yl)-7,8,9,10-tetrahydro-6H-azepino[1,2-a]indole-9-carboxylate (0.13 g, 0.33 mmol, 25%). ES-MS (m/z): 391.8/393.8 (M+1).

### EXAMPLE 24A

### 3,4-Dichloro-11-(1H-pyrazol-4-yl)-7,8,9,10-tetrahydro-6H-azepino[1,2-a]indole-9-carboxylic acid

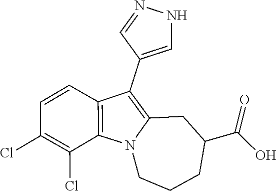 [SMILES: O=C(O)C1CCCn2c(c(-c3cn[nH]c3)c3ccc(Cl)c(Cl)c32)C1]

Add lithium hydroxide (0.04 g, 1.67 mmol) to ethyl 3,4-dichloro-11-(1H-pyrazol-4-yl)-7,8,9,10-tetrahydro-6H-azepino[1,2-a]indole-9-carboxylate (0.13 g, 0.33 mmol) in THF (1 mL), MeOH (0.66 mL) and water (0.33 mL) with stirring at RT. Heat to 50° C. for 1 hour. Neutralize via dropwise addition of 5 N aqueous HCl and concentrate under a stream of nitrogen gas. Triturate using ACN/water, collect by suction filtration and dry under vacuum to yield 3,4-dichloro-11-(1H-pyrazol-4-yl)-7,8,9,10-tetrahydro-6H-azepino[1,2-a]indole-9-carboxylic acid (0.75 g 0.2 mmol, 62%). ES-MS (m/z): 364.0/366.0 (M+1).

### EXAMPLES 24 & 25

### 3,4-Dichloro-11-(1H-pyrazol-4-yl)-7,8,9,10-tetrahydro-6H-azepino[1,2-a]indole-9-carboxylic acid

### Isomer 1 and Isomer 2

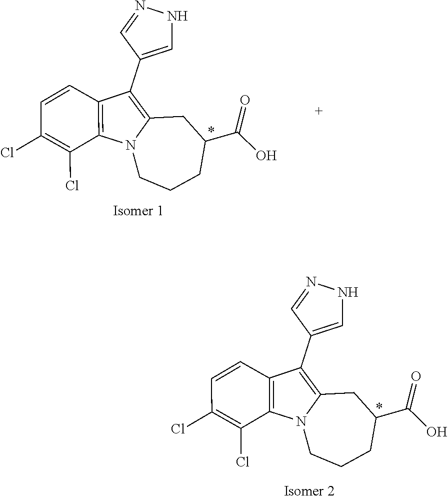 [SMILES: O=C(O)C1CCCn2c(c(-c3cn[nH]c3)c3ccc(Cl)c(Cl)c32)C1 ; O=C(O)C1CCCn2c(c(-c3cn[nH]c3)c3ccc(Cl)c(Cl)c32)C1]

Purify 3,4-dichloro-11-(1H-pyrazol-4-yl)-7,8,9,10-tetrahydro-6H-azepino[1,2-a]indole-9-carboxylic acid (0.075 g, 0.20 mmol) by supercritical fluid chromatography to yield the titled compounds (Isomer 1-26 mgs, 0.069 mmol, 35%; Isomer 2-28 mgs, 0.073 mmol, 37%). Rₜ=1.56 (99% ee) and 2.11 (99% ee) minutes. Column: Chiralcel OJ-H, 21×150 mm; Mobile Phase: 20% MeOH (w/0.5% DMEA): 80% CO₂; Flow Rate: 80 mL/min; Column temperature: 40° C.; Detection: 225 nM. ES-MS (m/z): 363.6/365.6 (M+1). ¹H NMR (400 MHz, DMSO): 13.06-12.98 (m, 1H), 7.79 (s, 2H), 7.46 (d, J=8.5 Hz, 1H), 7.22 (d, J=8.4 Hz, 1H), 5.42-5.33 (m, 1H), 4.34-4.28 (m, 1H), 3.46-3.43 (m, 2H), 3.07-2.84 (m, 1H), 2.34 (dt, J=7.2, 7.2 Hz, 1H), 2.18-2.09 (m, 2H), 1.88-1.79 (m, 1H), 1.64-1.56 (m, 1H).

### SCHEME FOR EXAMPLES 26 & 27

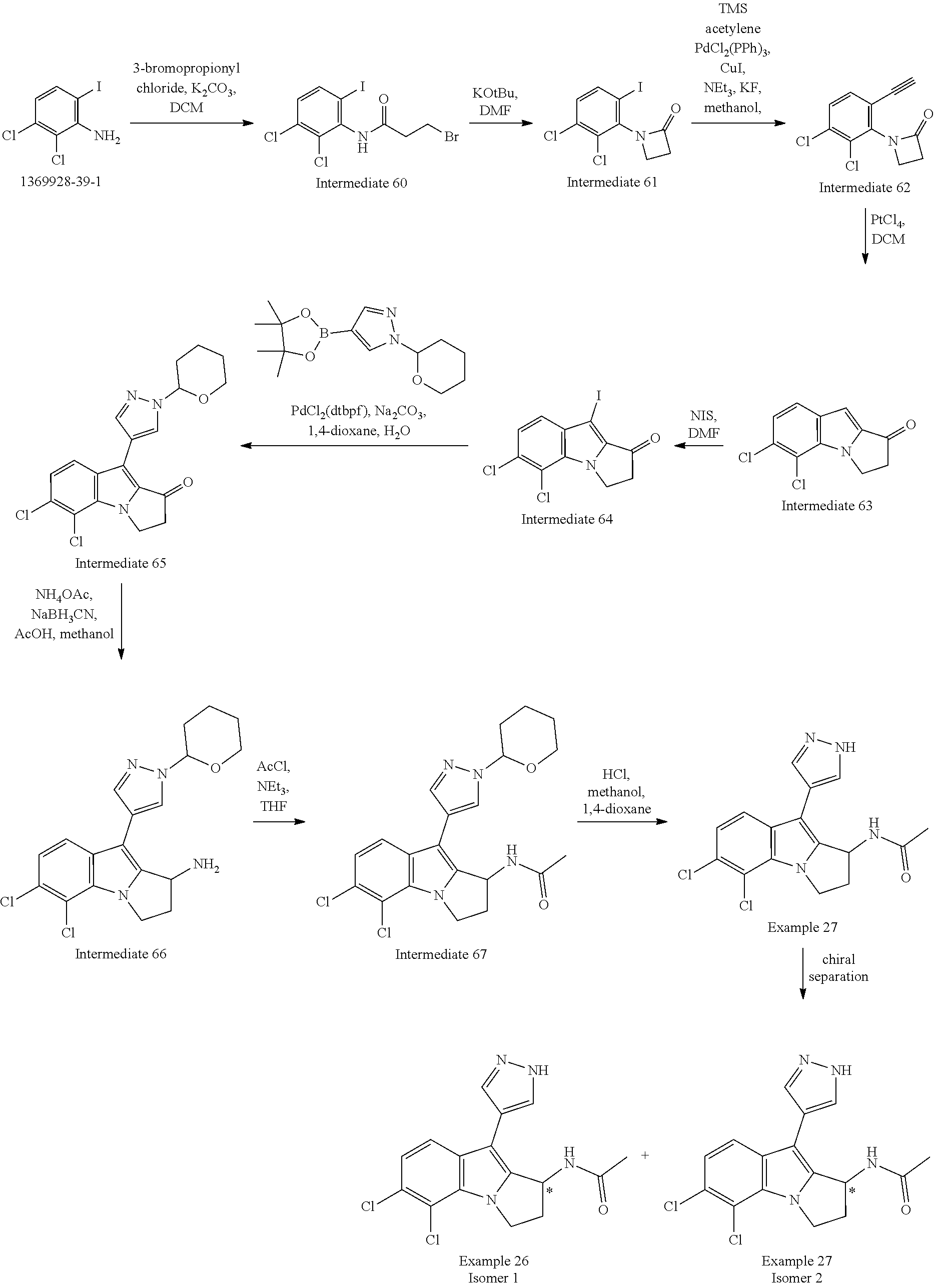

### Intermediate 60

### 3-Bromo-N-(2,3-dichloro-6-iodo-phenyl)propenamide

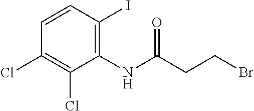 [SMILES: O=C(CCBr)Nc1c(I)ccc(Cl)c1Cl]

Add 3-Bromopropionyl chloride (8.8 mL, 87.5 mmol) to 2,3-dichloro-6-iodo-aniline (21 g, 73.0 mmol) and potassium carbonate (12 g, 87.5 mmol) in DCM (300 mL) with stirring, under nitrogen at 0° C. Allow to warm to RT and heat to 40° C. for 16 hours. Cool to RT, poured into water, and extract with DCM (3×). Dry combined organics over anhydrous magnesium sulfate, filter, and concentrate. Triturate from diethyl ether. Collect the solid by suction filtration and dry under vacuum to yield 3-bromo-N-(2,3-dichloro-6-iodo-phenyl)propanamide (18.3 g, 43.5 mmol, 59%). ¹H NMR (400 MHz, DMSO): 10.18 (s, 1H), 7.90 (d, J=8.6 Hz, 1H), 7.40 (d, J=8.6 Hz, 1H), 3.73 (t, J=6.4 Hz, 2H), 3.00 (td, J=6.4, 1.3 Hz, 2H).

### Intermediate 61

### 1-(2,3-Dichloro-6-iodo-phenyl)azetidin-2-one

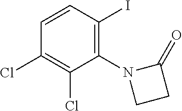 [SMILES: O=C1CCN1c1c(I)ccc(Cl)c1Cl]

Add sodium tert-butoxide (3.7 g, 38.4 mmol) to 3-bromo-N-(2,3-dichloro-6-iodo-phenyl)propanamide (14.75 g; 34.9 mmol) in DMF (100 mL) with stirring, under nitrogen at 0° C. Allow to warm to RT and stir for 3 hours. Pour into water and extract EtOAc (3×). Wash combined organic extracts with water and brine (2×), dry over anhydrous magnesium sulfate, filter, and concentrate. Triturate from diethyl ether/hexanes and collect the solid by suction filtration to yield 1-(2,3-dichloro-6-iodo-phenyl)azetidin-2-one (8.7 g, 25.7 mmol, 74%). ¹H NMR (400 MHz, DMSO): 7.93 (d, J=8.6 Hz, 1H), 7.49 (d, J=8.7 Hz, 1H), 3.72 (s, 2H), 3.18 (t, J=4.4 Hz, 2H).

### Intermediate 62

### 1-(2,3-Dichloro-6-ethynyl-phenyl)azetidin-2-one

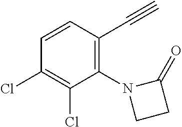

Add bis(triphenylphosphine)palladium(II) dichloride (0.80 g, 1.14 mmol) and cuprous iodide (0.43 g, 2.26 mmol) to 1-(2,3-dichloro-6-iodo-phenyl)azetidin-2-one (7.75 g, 22.7 mmol) and trimethylsilylacetylene (3.3 g, 34.0 mmol) in triethylamine (75 mL) with stirring, under nitrogen at RT. Heat to 50° C. for 5 hours. Concentrate and add MeOH (100 mL) and potassium fluoride dihydrate (6.5 g, 69.0 mmol). Stir at RT for 16 hours. Filter through diatomaceous earth and concentrate under reduced pressure. Purify by flash chromatography (EtOAc/hexanes) to yield 1-(2,3-dichloro-6-ethynyl-phenyl)azetidin-2-one (3.3 g, 14.0 mmol, 61%). ¹H NMR (400 MHz, DMSO): 7.70 (d, J=8.5 Hz, 1H), 7.58 (d, J=8.5 Hz, 1H), 4.69 (s, 1H), 3.78 (t, J=4.5 Hz, 2H), 3.18 (t, J=4.5 Hz, 2H).

### Intermediate 63

### 7,8-Dichloro-1,2-dihydropyrrolo[1,2-a]indol-3-one

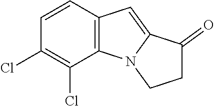 [SMILES: O=C1CCn2c1cc1ccc(Cl)c(Cl)c12]

Add platinum(IV) chloride (0.45 g, 1.33 mmol) to 1-(2,3-dichloro-6-ethynyl-phenyl)azetidin-2-one (3.3 g, 14.0 mmol) in 1,2-dichloroethane (700 mL) with stirring and under O₂. Heat to 85° C. for 16 hours. Concentrate and purify by flash chromatography (DCM) to yield 7,8-dichloro-1,2-dihydropyrrolo[1,2-a]indol-3-one (2.4 g 10.0 mmol, 73%). ¹H NMR (400 MHz, DMSO): 7.76 (d, J=8.7 Hz, 1H), 7.33 (d, J=8.7 Hz, 1H), 7.08 (s, 1H), 4.83 (t, J=6.2 Hz, 2H), 3.19 (t, J=6.2 Hz, 2H).

### Intermediate 64

### 7,8-Dichloro-4-iodo-1,2-dihydropyrrolo[1,2-a]indol-3-one

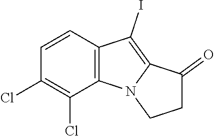 [SMILES: O=C1CCn2c1c(I)c1ccc(Cl)c(Cl)c12]

Add N-iodosuccinimide (2.8 g, 12.4 mmol) to 7,8-dichloro-1,2-dihydropyrrolo[1,2-a]indol-3-one (2.4 g, 10.0 mmol) in DMF (50 mL) with stirring, under nitrogen at RT. Heat to 60° C. for 16 hours. Pour into saturated aqueous sodium thiosulfate and extract with EtOAc (3×). Wash combined organic extracts with water and brine, dry over anhydrous magnesium sulfate, filter, and concentrate. Triturate from diethyl ether/hexanes and collect the solid by suction filtration to yield 7,8-dichloro-4-iodo-1,2-dihydropyrrolo[1,2-a]indol-3-one (3.3 g, 9.0 mmol, 90%). ES-MS (m/z): 365.5/367.5 (M+1).

### Intermediate 65

### 5,6-Dichloro-9-(1-(tetrahydro-2H-pyran-2-yl)-1H-pyrazol-4-yl)-2,3-dihydro-1H-pyrrolo[1,2-a]indol-1-one

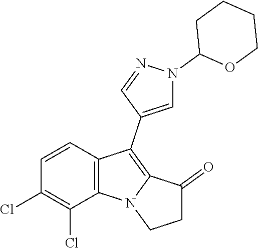 [SMILES: O=C1CCn2c1c(-c1cnn(C3CCCCO3)c1)c1ccc(Cl)c(Cl)c12]

Add 1,1′-Bis(di-tert-butylphosphino)ferrocene-palladium dichloride (1.2 g, 1.84 mmol) to a stirred solution of 1-(tetrahydro-2H-pyran-2-yl)-4-(4,4,5,5-tetramethyl-1,3,2-dioxaborolan-2-yl)-1H-pyrazole (5.1 g, 18.3 mmol), 7,8-dichloro-4-iodo-1,2-dihydropyrrolo[1,2-a]indol-3-one (3.3 g, 9.0 mmol) and sodium carbonate (2.9 g, 27.3 mmol) in 1,4-dioxane (50 mL) and water (10 mL) at RT. Degas with a stream of nitrogen gas for 10 min and heat to 90° C. for 3-4 hrs. Cool to RT, pour into water, and extract with EtOAc (3×). Wash the combined organics with brine, dry over anhydrous magnesium sulfate, filter, and concentrate. Purify by flash chromatography (EtOAc/dichloromethane) to yield 5,6-dichloro-9-(1-(tetrahydro-2H-pyran-2-yl)-1H-pyrazol-4-yl)-2,3-dihydro-1H-pyrrolo[1,2-a]indol-1-one (1.45 g, 3.7 mmol, 40%). ES-MS (m/z): 390.2/392.2 (M+1).

### Intermediate 66

### 5,6-Dichloro-9-(1-(tetrahydro-2H-pyran-2-yl)-1H-pyrazol-4-yl)-2,3-dihydro-1H-pyrrolo[1,2-a]indol-1-amine

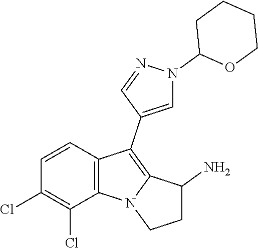 [SMILES: NC1CCn2c1c(-c1cnn(C3CCCCO3)c1)c1ccc(Cl)c(Cl)c12]

Add sodium cyanoborohydride (0.065 g, 1.03 mmol) to 5,6-dichloro-9-(1-(tetrahydro-2H-pyran-2-yl)-1H-pyrazol-4-yl)-2,3-dihydro-1H-pyrrolo[1,2-a]indol-1-one (0.2 g, 0.50 mmol), ammonium acetate (0.77 g, 10.0 mmol) and acetic acid (0.03 mL, 0.52 mmol) in MeOH (5 mL). Stir in a microwave vial under nitrogen at RT. Seal vial and heat to 120° C. in the microwave for 2 hours. Cool to RT, pour into saturated aqueous sodium bicarbonate, and extract with EtOAc (3×). Dry the combined organics over anhydrous magnesium sulfate, filter and concentrate to yield 5,6-dichloro-9-(1-(tetrahydro-2H-pyran-2-yl)-1H-pyrazol-4-yl)-2,3-dihydro-1H-pyrrolo[1,2-a]indol-1-amine (0.17 g, 0.423 mmol, 85%). ES-MS (m/z): 391.0/393.0 (M+1).

### Intermediate 67

### N-(5,6-Dichloro-9-(1-(tetrahydro-2H-pyran-2-yl)-1H-pyrazol-4-yl)-2,3-dihydro-1H-pyrrolo[1,2-a]indol-1-yl)acetamide

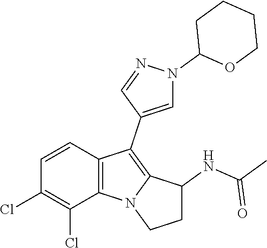 [SMILES: CC(=O)NC1CCn2c1c(-c1cnn(C3CCCCO3)c1)c1ccc(Cl)c(Cl)c12]

Add acetyl chloride (0.03 mL, 0.42 mmol) to 5,6-dichloro-9-(1-(tetrahydro-2H-pyran-2-yl)-1H-pyrazol-4-yl)-2,3-dihydro-1H-pyrrolo[1,2-a]indol-1-amine (0.17 g, 0.423 mmol) and triethylamine (0.09 mL, 1.49 mmol) in THF (4 mL) with stirring, under nitrogen at RT. Stir for 1 hour. Pour into water and extract with EtOAc (3×). Wash the combined organics with brine, dry over anhydrous magnesium sulfate, filter, and concentrate to yield N-(5,6-dichloro-9-(1-(tetrahydro-2H-pyran-2-yl)-1H-pyrazol-4-yl)-2,3-dihydro-1H-pyrrolo[1,2-a]indol-1-yl)acetamide (0.18 g, 0.40 mmol, 93%). ES-MS (m/z): 433.0/435.0 (M+1).

### EXAMPLE 27A

### N-(5,6-Dichloro-9-(1H-pyrazol-4-yl)-2,3-dihydro-1H-pyrrolo[1,2-a]indol-1-yl)acetamide

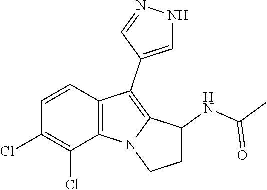 [SMILES: CC(=O)NC1CCn2c1c(-c1cn[nH]c1)c1ccc(Cl)c(Cl)c12]

Add HCl (4.0 mol/L) in dioxane (1.0 mL, 4.0 mmol) to N-(5,6-dichloro-9-(1-(tetrahydro-2H-pyran-2-yl)-1H-pyrazol-4-yl)-2,3-dihydro-1H-pyrrolo[1,2-a]indol-1-yl)acetamide (0.18 g, 0.40 mmol) in 1,4-dioxane (3 mL) and MeOH (1 mL) with stirring, under nitrogen at RT. Stir for 1 hour. Pour into saturated aqueous sodium bicarbonate and extract with EtOAc (3×). Dry the combined organics over anhydrous magnesium sulfate, filter, and concentrate. Purify by reverse phase flash chromatography (acetonitrile/water/0.1% formic acid) to yield N-(5,6-dichloro-9-(1H-pyrazol-4-yl)-2,3-dihydro-1H-pyrrolo[1,2-a]indol-1-yl)acetamide (0.066 g, 0.183 mmol, 46%). ES-MS (m/z): 349.0/351.0 (M+1).

### EXAMPLES 26 & 27

### N-(5,6-Dichloro-9-(1H-pyrazol-4-yl)-2,3-dihydro-1H-pyrrolo[1,2-a]indol-1-yl)acetamide

### Isomer 1 and Isomer 2

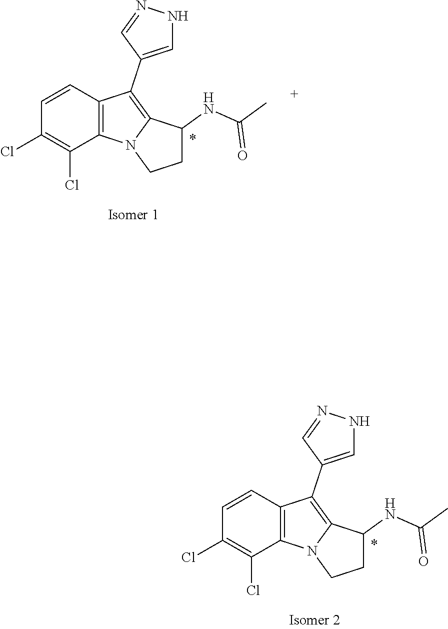 [SMILES: CC(=O)NC1CCn2c1c(-c1cn[nH]c1)c1ccc(Cl)c(Cl)c12 ; CC(=O)NC1CCn2c1c(-c1cn[nH]c1)c1ccc(Cl)c(Cl)c12]

Purify N-[7,8-dichloro-4-(1H-pyrazol-4-yl)-2,3-dihydro-1H-pyrrolo[1,2-a]indol-3-yl]acetamide (0.066 g, 0.183 mmol) by supercritical fluid chromatography to yield the titled compounds (Isomer 1-26 mg, 0.039 mmol, 39%; Isomer 2-45 mgs 0.073 mmol, 46%). Rₜ=1.52 (99% ee) and 2.20 (97% ee) minutes. Column: Chiralpak AD-H, 21×150 mm; Mobile Phase: 30% EtOH: 70% CO₂; Flow Rate: 80 mL/min; Column temperature: 40° C.; Detection: 240 nM. ES-MS (m/z): 349.2/351.2 (M+1). ¹H NMR (400 MHz, DMSO): 12.98-12.90 (m, 1H), 8.53 (d, J=8.7 Hz, 1H), 8.01-7.94 (m, 1H), 7.80-7.73 (m, 2H), 7.23 (d, J=8.6 Hz, 1H), 5.57 (td, J=8.2, 3.2 Hz, 1H), 4.56-4.53 (m, 2H), 2.96-2.87 (m, 1H), 2.34-2.30 (m, 1H), 1.81 (s, 3H).

### SCHEME FOR EXAMPLE 28

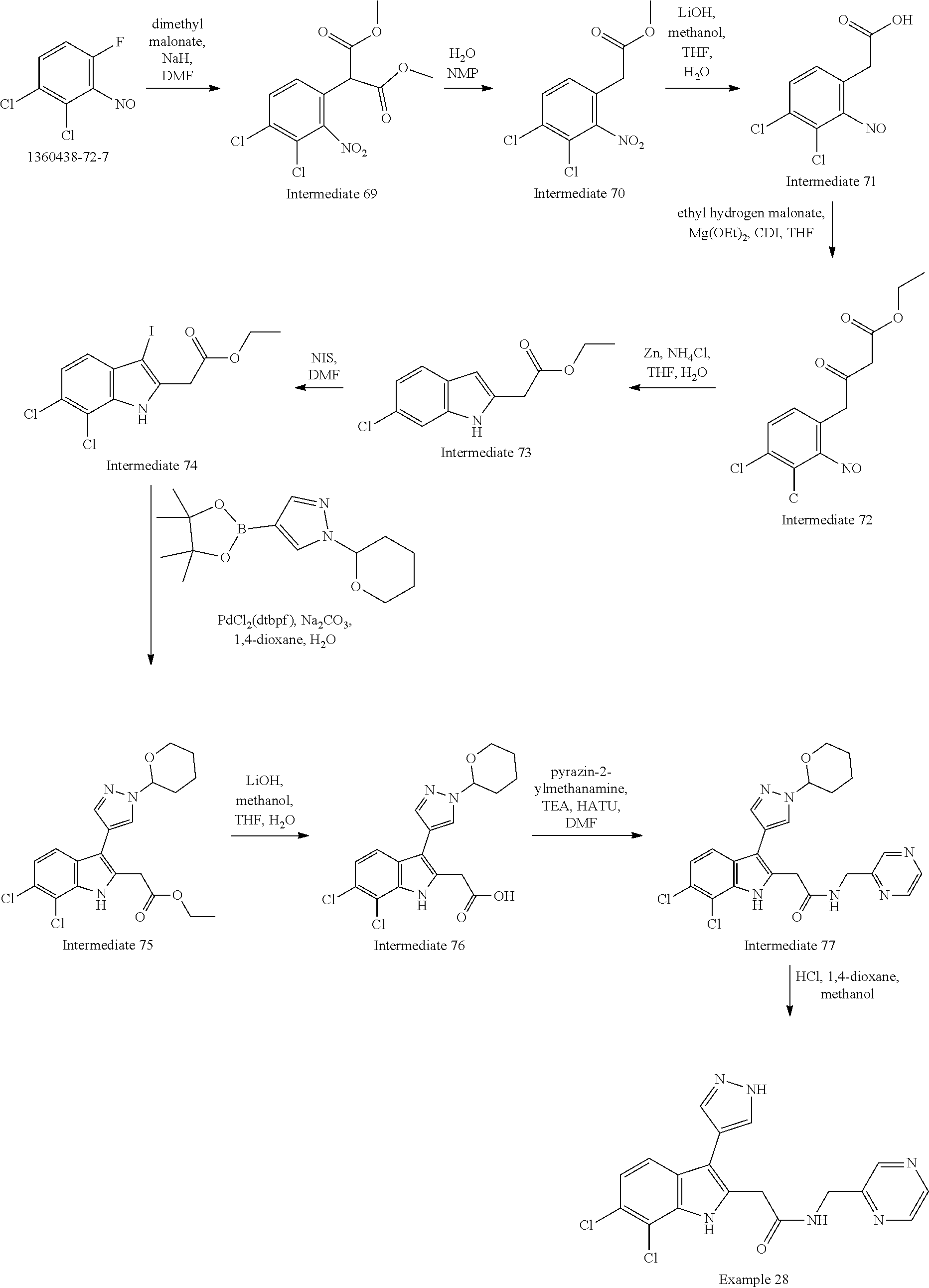

### Intermediate 69

### Dimethyl 2-(3,4-dichloro-2-nitro-phenyl)propanedioate

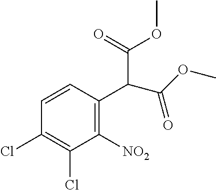 [SMILES: COC(=O)C(C(=O)OC)c1ccc(Cl)c(Cl)c1[N+](=O)[O-]]

Add sodium hydride (60 mass %) in mineral oil (2.4 g, 60.3 mmol) portion wise to dimethyl malonate (8.5 g, 65.3 mmol) in DMF (150 mL) with stirring, under nitrogen at 0° C. Stir 5 mins and remove the ice bath. Stir at RT for 30 min. Add 1,2-dichloro-4-fluoro-3-nitrobenzene (11.0 g, 50.3 mmol) in DMF (5 mL) and continue stirring at RT for 16 hours. Pour into ice/conc. HCl (25 mL) with stirring. Neutralize with 1 N NaOH and stir until a precipitate forms. Collect solids by suction filtration, rinse with water and dry under vacuum to yield dimethyl 2-(3,4-dichloro-2-nitro-phenyl)propanedioate (13.8 g, 41.1 mmol, 82%). ES-MS (m/z): 320.0/322.0 (M−1, negative ionization mode).

### Intermediate 70

### Methyl 2-(3,4-dichloro-2-nitro-phenyl)acetate

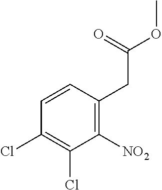 [SMILES: COC(=O)Cc1ccc(Cl)c(Cl)c1[N+](=O)[O-]]

Add water (20 mL) to dimethyl 2-(3,4-dichloro-2-nitro-phenyl)propanedioate (13.5 g, 40.2 mmol) in NMP (80 mL) with stirring at RT. Heat to reflux for 3 hours. Cool to RT, pour into water and extract with EtOAc (3×). Wash combined organics with water and brine, dry over anhydrous magnesium sulfate, filter, and concentrate. Purify by flash chromatography (EtOAc/hexanes) to yield methyl 2-(3,4-dichloro-2-nitro-phenyl)acetate (5.75 g, 21.8 mmol, 52%). ¹H NMR (400 MHz, DMSO): 7.97 (d, J=8.5 Hz, 1H), 7.63 (d, J=8.5 Hz, 1H), 3.85 (s, 2H), 3.63 (s, 3H).

### Intermediate 71

### 2-(3,4-Dichloro-2-nitro-phenyl)acetic acid

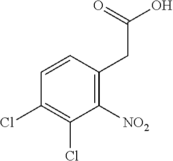 [SMILES: O=C(O)Cc1ccc(Cl)c(Cl)c1[N+](=O)[O-]]

Add lithium hydroxide (2.6 g, 110 mmol) to methyl 2-(3,4-dichloro-2-nitro-phenyl)acetate (5.7 g, 21.7 mmol) in THF (60 mL), methanol (40 mL) and water (20 mL) with stirring at RT. Heat to 60° C. for 2-3 hours. Cool to RT, pour into 1N aqueous HCl and extract with DCM (3×). Dry the combined organics over anhydrous magnesium sulfate, filter and concentrate to yield methyl 2-(3,4-dichloro-2-nitro-phenyl)acetate (5.35 g, 21.4 mmol, 95%). ¹H NMR (400 MHz, DMSO): 12.85 (s, 1H), 7.94 (d, J=8.4 Hz, 1H), 7.61 (d, J=8.5 Hz, 1H), 3.73 (s, 2H).

### Intermediate 72

### Ethyl 4-(3,4-dichloro-2-nitro-phenyl)-3-oxo-butanoate

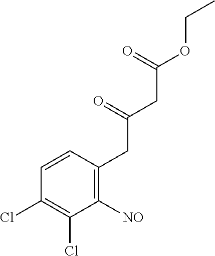

Add magnesium ethoxide (1.2 g, 10.0 mmol) to ethyl hydrogen malonate (2.8 g, 20 mmol) in THF (50 mL) with stirring, under nitrogen at RT. Stir at RT for 2.5 hours. In a separate flask, add 1,1′-carbonyldiimidazole (3.7 g, 22 mmol) to 3,4-dichloro-2-nitrophenylacetic acid (5.3 g, 20 mmol) in THF (100 mL) with stirring, under nitrogen at RT. Heat to 40° C. for 2 hours. Cool to RT and add the crude magnesium salt in one portion. Stir at RT for 16 hours. Concentrate and redissolve in DCM. Wash with 0.5 N aqueous HCl, water and brine. Dry over anhydrous magnesium sulfate, filter, and concentrate. Purify by flash chromatography (EtOAc/hexanes) to yield ethyl 4-(3,4-dichloro-2-nitro-phenyl)-3-oxo-butanoate (2.63 g, 7.89 mmol, 39%). ¹H NMR (400 MHz, DMSO): 7.94 (d, J=8.5 Hz, 1H), 7.49 (d, J=8.5 Hz, 1H), 4.14-4.08 (m, 4H), 3.71 (s, 2H), 1.20 (t, J=7.1 Hz, 3H).

### Intermediate 73

### Ethyl 2-(6,7-dichloro-1H-indol-2-yl)acetate

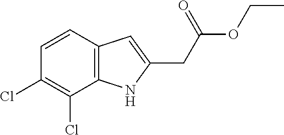 [SMILES: CCOC(=O)Cc1cc2ccc(Cl)c(Cl)c2[nH]1]

Add zinc powder (15 g) to a stirred solution of ethyl 4-(3,4-dichloro-2-nitro-phenyl)-3-oxo-butanoate (2 g, 6.0 mmol) in THF (15 mL) and saturated aqueous ammonium chloride (15 mL) at RT. Stir vigorously for 2 hours. Quench with 5 mL of saturated aqueous potassium bicarbonate and filter through diatomaceous earth. Dilute filtrate with water and extract with EtOAc (3×). Dry over anhydrous magnesium sulfate, filter, and concentrate. Purify by flash chromatography (EtOAc/hexanes) to yield ethyl 2-(6,7-dichloro-1H-indol-2-yl)acetate (1.57 g, 5.77 mmol, 96%). ¹H NMR (400 MHz, DMSO): 11.54 (s, 1H), 7.47 (d, J=8.4 Hz, 1H), 7.16 (d, J=8.4 Hz, 1H), 6.43-6.42 (m, 1H), 4.13 (q, J=7.1 Hz, 2H), 3.88 (s, 2H), 1.22 (t, J=7.1 Hz, 3H).

### Intermediate 74

### Ethyl 2-(6,7-dichloro-3-iodo-1H-indol-2-yl)acetate

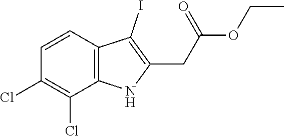 [SMILES: CCOC(=O)Cc1[nH]c2c(Cl)c(Cl)ccc2c1I]

Add N-iodosuccinimide (2.9 g, 8.7 mmol) to ethyl 2-(6,7-dichloro-1H-indol-2-yl)acetate (2.0 g, 7.35 mmol) in DMF (30 mL) with stirring, under nitrogen at RT. Stir for 16 hours. Pour into saturated aqueous sodium thiosulfate and extract with EtOAc (3×). Wash the combined organics with water and brine, dry over anhydrous magnesium sulfate, filter, and concentrate. Purify by flash chromatography (EtOAc/hexanes) to yield ethyl 2-(6,7-dichloro-3-iodo-1H-indol-2-yl)acetate (2.3 g, 5.78 mmol, 79%). ES-MS (m/z): 398.0/400.0 (M+1).

### Intermediate 75

### Ethyl 2-[6,7-dichloro-3-(1-tetrahydropyran-2-ylpyrazol-4-yl)-1H-indol-2-yl]acetate

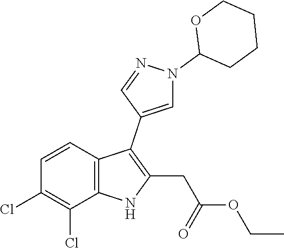 [SMILES: CCOC(=O)Cc1[nH]c2c(Cl)c(Cl)ccc2c1-c1cnn(C2CCCCO2)c1]

Add 1,1′-Bis(di-tert-butylphosphino)ferrocene-palladium dichloride (0.75 g, 1.13 mmol) to a stirred solution of 1-(tetrahydro-2H-pyran-2-yl)-4-(4,4,5,5-tetramethyl-1,3,2-dioxaborolan-2-yl)-1H-pyrazole (3.3 g, 11.8 mmol), 2-(6,7-dichloro-3-iodo-1H-indol-2-yl)acetate (2.3 g 5.78 mmol) and sodium carbonate (1.8 g, 17.0 mmol) in 1,4-dioxane (30 mL) and water (7 mL) at RT. Degas with a stream of nitrogen gas for 10 min and heat to 90° C. for 3-4 hours. Cool to RT, pour into water and extract with EtOAc (3×). Wash combined organics with brine, dry over anhydrous magnesium sulfate, filter, and concentrate. Purify by flash chromatography (EtOAc/dichloromethane) to yield ethyl 2-[6,7-dichloro-3-(1-tetrahydropyran-2-ylpyrazol-4-yl)-1H-indol-2-yl]acetate (2.07 g, 4.75 mmol, 82%). ES-MS (m/z): 422.2/424.2 (M+1).

### Intermediate 76

### 2-[6,7-Dichloro-3-(1-tetrahydropyran-2-ylpyrazol-4-yl)-1H-indol-2-yl]acetic acid

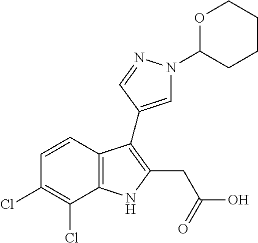 [SMILES: O=C(O)Cc1[nH]c2c(Cl)c(Cl)ccc2c1-c1cnn(C2CCCCO2)c1]

Add lithium hydroxide (0.16 g, 6.68 mmol) to 2-[6,7-dichloro-3-(1-tetrahydropyran-2-ylpyrazol-4-yl)-1H-indol-2-yl]acetate (0.58 g, 1.34 mmol) in THF (3 mL), methanol (2 mL) and water (1 mL) with stirring at RT. Stir for 2 hours. Neutralize to pH 6 with 1N aqueous HCl. Pour into water and extract with EtOAc (3×). Dry combined organics over magnesium sulfate, filter, and concentrate. Triturate from diethyl ether/hexanes, collect the solid by suction filtration and dry under vacuum to yield 2-[6,7-dichloro-3-(1-tetrahydropyran-2-ylpyrazol-4-yl)-1H-indol-2-yl]acetic acid (0.23 mg, 0.57 mmol, 43%). ES-MS (m/z): 393.6/395.6 (M+1).

### Intermediate 77

### 2-[6,7-Dichloro-3-(1-tetrahydropyran-2-ylpyrazol-4-yl)-1H-indol-2-yl]-N-(pyrazin-2-ylmethyl)acetamide

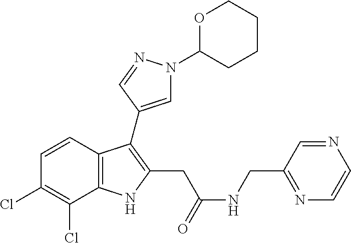 [SMILES: O=C(Cc1[nH]c2c(Cl)c(Cl)ccc2c1-c1cnn(C2CCCCO2)c1)NCc1cnccn1]

Add triethylamine (0.14 mL, 1.0 mmol) and HATU (0.092 g, 0.237 mmol) to 2-[6,7-dichloro-3-[1-(tetrahydropyran-2-ylpyrazol-4-yl)-1H-indol-2-yl]acetic acid (0.08 g, 0.196 mmol) and pyrazin-2-ylmethanamine (0.025 mL, 0.216 mmol) in DMF (3 mL) with stirring, under nitrogen at RT. Stir for 16 hours. Pour into water and extract with EtOAc (3×). Wash the combined organics with water and brine (2×), dry over anhydrous magnesium sulfate, filter, and concentrate. Purify by flash chromatography (MeOH/EtOAc/DCM) to yield 2-[6,7-dichloro-3-(1-tetrahydropyran-2-ylpyrazol-4-yl)-1H-indol-2-yl]-N-(pyrazin-2-ylmethyl)acetamide (0.078 g, 0.156 mmol, 79%). ES-MS (m/z): 484.8/486.8 (M+1).

### EXAMPLE 28

### 2-[6,7-Dichloro-3-(1H-pyrazol-4-yl)-1H-indol-2-yl]-N-(pyrazin-2-ylmethyl)acetamide

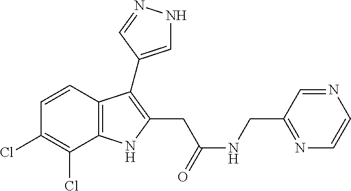 [SMILES: O=C(Cc1[nH]c2c(Cl)c(Cl)ccc2c1-c1cn[nH]c1)NCc1cnccn1]

Add HCl (4.0 mol/L) in dioxane (0.375 mL, 1.5 mmol) to 2-[6,7-dichloro-3-(1-tetrahydropyran-2-ylpyrazol-4-yl)-1H-indol-2-yl]-N-(pyrazin-2-ylmethyl)acetamide (0.075 g, 0.15 mmol) in 1,4-dioxane (3 mL) and MeOH (1 mL) with stirring, under nitrogen at RT. Stir for 1 hour. Pour into saturated aqueous sodium bicarbonate and extract with EtOAc (3×). Dry the combined organics over anhydrous magnesium sulfate, filter and concentrate to yield 2-[6,7-dichloro-3-(1H-pyrazol-4-yl)-1H-indol-2-yl]-N-(pyrazin-2-ylmethyl)acetamide (0.049 g, 0.118 mmol, 79%). ES-MS (m/z): 401.0/403.0 (M+1). ¹H NMR (400 MHz, DMSO): 13.00 (s, 1H), 11.54 (s, 1H), 8.66-8.58 (m, 4H), 8.06-8.00 (m, 1H), 7.88-7.87 (m, 1H), 7.58 (d, J=8.5 Hz, 1H), 7.21 (d, J=8.4 Hz, 1H), 4.48 (d, J=5.8 Hz, 2H), 3.81 (s, 2H).

### SCHEME FOR EXAMPLE 29

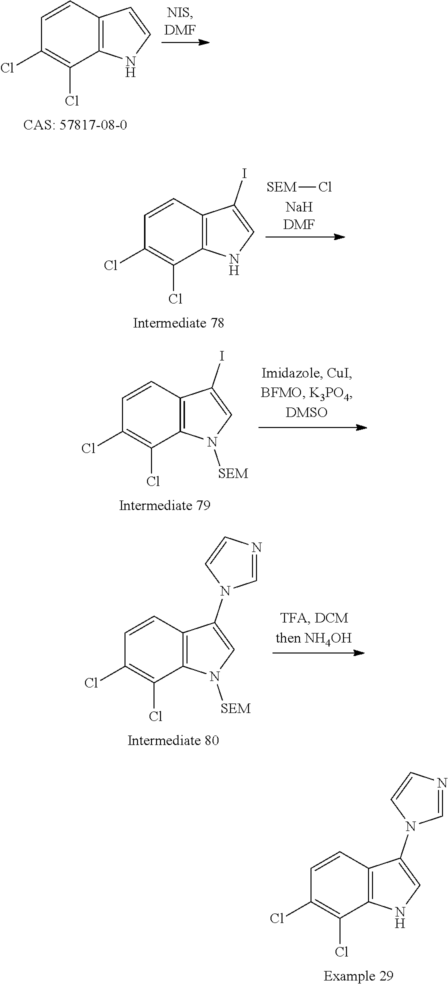 [SMILES: Clc1ccc2cc[nH]c2c1Cl ; Clc1ccc2c(I)c[nH]c2c1Cl ; C[Si](C)(C)CCOCCl ; C[Si](C)(C)CCOCn1cc(I)c2ccc(Cl)c(Cl)c21 ; C[Si](C)(C)CCOCn1cc(-n2ccnc2)c2ccc(Cl)c(Cl)c21 ; Clc1ccc2c(-n3ccnc3)c[nH]c2c1Cl]

### Intermediate 78

### 6,7-Dichloro-3-iodo-1H-indole

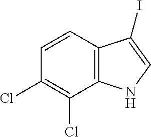 [SMILES: Clc1ccc2c(I)c[nH]c2c1Cl]

Dissolve 6,7-dichloro-1H-indole (500 mg, 2.55 mmol) in DMF (10 mL). Add NIS (710 mg, 3.06 mmol) and stir at 25° C. for 1 hour. Quench with saturated aqueous Na₂S₂O₃ and extract with EtOAc. Wash with saturated aqueous NaHCO₃, dry over anhydrous sodium sulfate, filter and concentrate to yield 6,7-dichloro-3-iodo-1H-indole (1.00 g, 2.40 mmol, 94% Yield) as yellow oil.

### Intermediate 79

### 2-[(6,7-Dichloro-3-iodo-indol-1-yl)methoxy]ethyl-trimethyl-silane

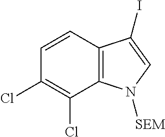 [SMILES: C[Si](C)(C)CCOCn1cc(I)c2ccc(Cl)c(Cl)c21]

Dissolve 6,7-dichloro-3-iodo-1H-indole (1.00 g, 2.40 mmol) in DMF (10 mL). Add NaH (145 mg, 3.63 mmol, 60% dispersion in mineral oil) in portions at 0° C. and stir for 1 hour. Add SEM-Cl (600 mg, 0.67 mL, 3.60 mmol) and stir at RT overnight. Quench with water, extract with EtOAc, wash with brine, dry over saturated sodium sulfate, filter, and concentrate. Purify by flash column chromatography (EtOAc/PE) to yield 2-[(6,7-dichloro-3-iodo-indol-1-yl)methoxy]ethyl-trimethyl-silane (1.12 g, 2.28 mmol, 95% Yield) as yellow oil.

### Intermediate 80

### 2-[(6,7-Dichloro-3-imidazol-1-yl-indol-1-yl)methoxy]ethyl-trimethyl-silane

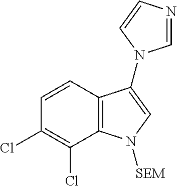 [SMILES: C[Si](C)(C)CCOCn1cc(-n2ccnc2)c2ccc(Cl)c(Cl)c21]

Suspend 2-[(6,7-dichloro-3-iodo-indol-1-yl)methoxy]ethyl-trimethyl-silane (550 mg, 1.12 mmol), imidazole (150 mg, 2.20 mmol), CuI (35 mg, 0.23 mmol), BFMO (60 mg, 0.23 mmol) and K₃PO₄ (500 mg, 2.31 mmol) in DMSO (12 mL). Degas and purge with N₂. Heat at 120° C. for 1.5 hours in the microwave. Cool to RT, dilute with water and extract with EtOAc. Wash with brine, dry over sodium sulfate, filter, and concentrate. Purify by flash column chromatography (EtOAc/PE) to yield 2-[(6,7-dichloro-3-imidazol-1-yl-indol-1-yl)methoxy]ethyl-trimethyl-silane (210 mg, 0.522 mmol, 47% Yield) as yellow gum. ES-MS (m/z): 382.1/384.0 (M+H).

### EXAMPLE 29

### 6,7-Dichloro-3-imidazol-1-yl-1H-indole

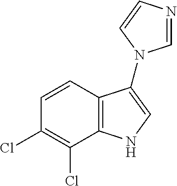 [SMILES: Clc1ccc2c(-n3ccnc3)c[nH]c2c1Cl]

Dissolve 2-[(6,7-dichloro-3-imidazol-1-yl-indol-1-yl)methoxy]ethyl-trimethyl-silane (210 mg, 0.549 mmol) in DCM (3 mL). Add TFA (1 mL) and stir at RT for 2 hours. Concentrate and redissolve in MeOH (1.5 mL). Add 28% aqueous ammonium hydroxide (1.5 mL) and stir 1 hour at RT. Filter and purify the filtrate by prep-HPLC to yield 6-bromo-7-6,7-dichloro-3-imidazol-1-yl-1H-indole (87 mg, 0.34 mmol, 66% Yield) as a white solid. ES-MS (m/z): 252.0/254.0 (M+H). ¹H NMR (400 MHz, DMSO): 12.26 (s, 1H), 8.77 (s, 1H), 8.01 (d, J=2.9 Hz, 1H), 7.87 (s, 1H), 7.62 (d, J=8.6 Hz, 1H), 7.53 (s, 1H), 7.36 (d, J=8.6 Hz, 1H).

### SCHEME FOR EXAMPLE 30

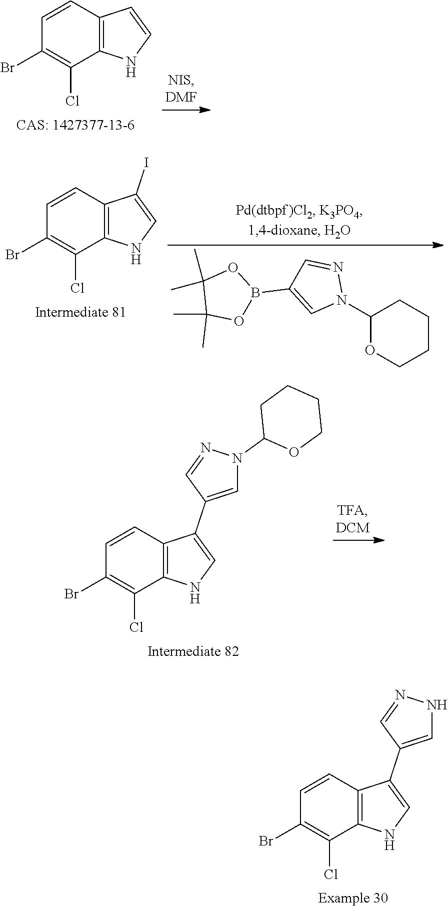 [SMILES: Clc1c(Br)ccc2cc[nH]c12 ; Clc1c(Br)ccc2c(I)c[nH]c12 ; CC1(C)OB(c2cnn(C3CCCCO3)c2)OC1(C)C ; Clc1c(Br)ccc2c(-c3cnn(C4CCCCO4)c3)c[nH]c12 ; Clc1c(Br)ccc2c(-c3cn[nH]c3)c[nH]c12]

### Intermediate 81

### 6-Bromo-7-chloro-3-iodo-1H-indole

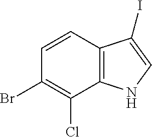 [SMILES: Clc1c(Br)ccc2c(I)c[nH]c12]

Dissolve 6-bromo-7-chloro-1H-indole (700 mg, 2.73 mmol) in DMF (50 mL) and add NIS (800 mg, 3.45 mmol). Stir 2 hours at RT. Quench with water and stir vigorously for 15 min. Dilute with EtOAc, wash with brine, dry over saturated sodium sulfate, filter, and concentrate to yield 6-bromo-7-chloro-3-iodo-1H-indole (600 mg, 1.60 mmol, 58% Yield).

### Intermediate 82

### 6-Bromo-7-chloro-3-(1-(tetrahydro-2H-pyran-2-yl)-1H-pyrazol-4-yl)-1H-indole

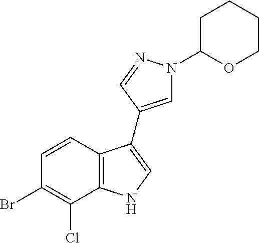 [SMILES: Clc1c(Br)ccc2c(-c3cnn(C4CCCCO4)c3)c[nH]c12]

Suspend 6-bromo-7-chloro-3-iodo-1H-indole (600 mg, 1.60 mmol), 1-(tetrahydro-2H-pyran-2-yl)-4-(4,4,5,5-tetramethyl-1,3,2-dioxaborolan-2-yl)-1H-pyrazole (460 mg, 1.60 mmol), Pd(dtbpf)Cl₂ (220 mg, 0.320 mmol) and K₃PO₄ (1.00 g, 4.80 mmol) in 1,4-dioxane (10 mL) and water (1 mL). Degas and purge with N₂ and heat at 90° C. for 1 hour. Cool to RT, dilute with EtOAc, wash sequentially with saturated aqueous sodium bicarbonate and brine, dry over sodium sulfate, filter, and concentrate. Purify by flash column chromatography (MeOH/DCM) to yield 6-bromo-7-chloro-3-(1-(tetrahydro-2H-pyran-2-yl)-1H-pyrazol-4-yl)-1H-indole (640 mg, 0.387 mmol, 24% Yield). ES-MS (m/z): 380.0/382.0/384.0 (M+H)

### EXAMPLE 30

### 6-bromo-7-chloro-3-(1H-pyrazol-4-yl)-1H-indole

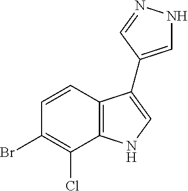 [SMILES: Clc1c(Br)ccc2c(-c3cn[nH]c3)c[nH]c12]

Suspend 6-bromo-7-chloro-3-(1-(tetrahydro-2H-pyran-2-yl)-1H-pyrazol-4-yl)-1H-indole (640 mg, 0.387 mmol) in DCM (5 mL). Add TFA (1.0 mL) and stir 2 hours at RT. Concentrate and dilute with EtOAc. Adjust to pH 8 with saturated aqueous NaHCO₃, wash with saturated aqueous sodium bicarbonate (×3), once with brine, dry over anhydrous sodium sulfate, filter, and concentrate. Purify by prep-HPLC to yield 6-bromo-7-chloro-3-(1H-pyrazol-4-yl)-1H-indole (Product, 72 mg, 0.24 mmol, 45% Yield) as a white solid. ES-MS (m/z): 295.7/297.7/299.6 (M+H). ¹H NMR (400 MHz, DMSO): 12.88 (br s, 1H), 11.65 (s, 1H), 8.00 (br s, 2H), 7.73 (d, J=8.5 Hz, 1H), 7.67 (d, J=2.1 Hz, 1H), 7.34 (d, J=8.5 Hz, 1H).

### SCHEME FOR EXAMPLE 31

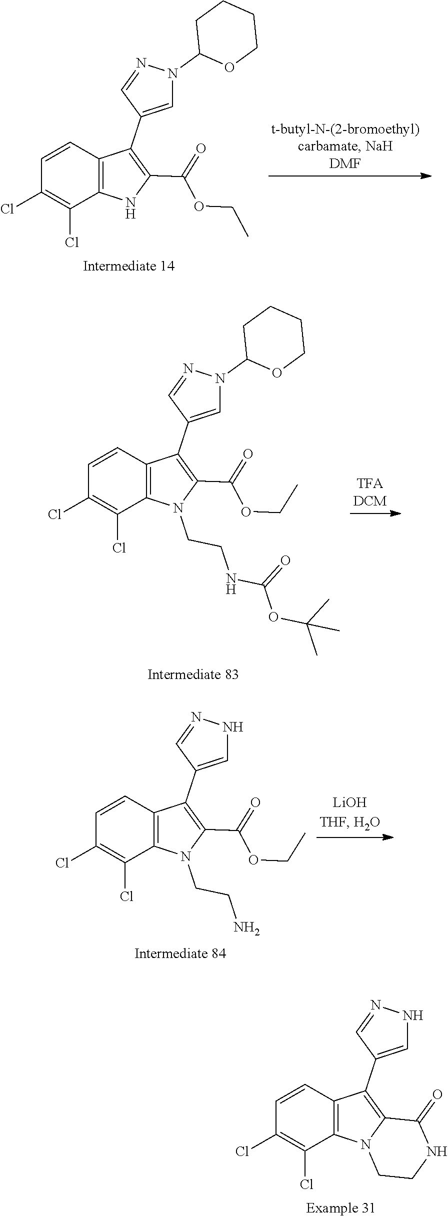 [SMILES: CCOC(=O)c1[nH]c2c(Cl)c(Cl)ccc2c1-c1cnn(C2CCCCO2)c1 ; CCOC(=O)c1c(-c2cnn(C3CCCCO3)c2)c2ccc(Cl)c(Cl)c2n1CCNC(=O)OC(C)(C)C ; CCOC(=O)c1c(-c2cn[nH]c2)c2ccc(Cl)c(Cl)c2n1CCN ; O=C1NCCn2c1c(-c1cn[nH]c1)c1ccc(Cl)c(Cl)c12]

### Intermediate 83

### Ethyl 1-[2-(tert-butoxycarbonylamino)ethyl]-6,7-dichloro-3-(1-tetrahydropyran-2-ylpyrazol-4-yl)indole-2-carboxylate

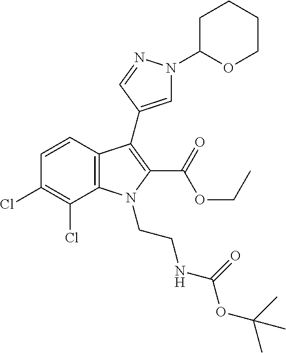 [SMILES: CCOC(=O)c1c(-c2cnn(C3CCCCO3)c2)c2ccc(Cl)c(Cl)c2n1CCNC(=O)OC(C)(C)C]

Dissolve ethyl 6,7-dichloro-3-(1-tetrahydropyran-2-ylpyrazol-4-yl)-1H-indole-2-carboxylate (300 mg, 0.514 mmol, 70% purity) in DMF (6 mL) and add NaH (10 mg, 0.25 mmol, 60% dispersion in mineral oil) at RT. Stir at RT for 0.5 hour, add tert-butyl N-(2-bromoethyl)carbamate (1.28 g, 5.14 mmol) and stir at 30° C. for 2 hours. Quench with 1N aqueous HCl, extract with EtOAc, wash with brine, dry over sodium sulfate, filter, and concentrate. Purify by prep-TLC (EtOAc/PE=1/1) to yield ethyl 1-[2-(tert-butoxycarbonylamino)ethyl]-6, 7-dichloro-3-(1-tetrahydropyran-2-ylpyrazol-4-yl)indole-2-carboxylate (330 mg, 92% Yield) as yellow oil. ES-MS (m/z): 551.1/553.0 (M+H).

### Intermediate 84

### Ethyl 1-(2-aminoethyl)-6,7-dichloro-3-(1H-pyrazol-4-yl)indole-2-carboxylate

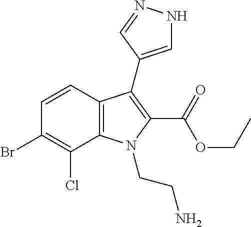 [SMILES: CCOC(=O)c1c(-c2cn[nH]c2)c2ccc(Br)c(Cl)c2n1CCN]

Dissolve ethyl 1-[2-(tert-butoxycarbonylamino)ethyl]-6, 7-dichloro-3-(1-tetrahydropyran-2-ylpyrazol-4-yl)indole-2-carboxylate (330 mg, 0.473 mmol) in DCM (0.5 mL). Add TFA (0.5 mL) and stir at RT for 2 hours. Concentrate to yield ethyl 1-(2-aminoethyl)-6,7-dichloro-3-(1H-pyrazol-4-yl)indole-2-carboxylate (200 mg, 0.430 mmol, 91% Yield, TFA salt) as yellow oil.

### EXAMPLE 31

### 6, 7-Dichloro-10-(1H-pyrazol-4-yl)-3, 4-dihydro-2H-pyrazino[1,2-a]indol-1-one

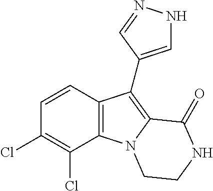 [SMILES: O=C1NCCn2c1c(-c1cn[nH]c1)c1ccc(Cl)c(Cl)c12]

Dissolve ethyl 1-(2-aminoethyl)-6, 7-dichloro-3-(1H-pyrazol-4-yl)indole-2-carboxylate (100 mg, 0.215 mmol) in THF (1 mL) and water (0.5 mL). Add LiOH·H₂O (52 mg, 2.1 mmol) and stir overnight at RT. Neutralize with 1N aqueous HCl and concentrate. Triturate the crude product with DMSO to yield 6, 7-dichloro-10-(1H-pyrazol-4-yl)-3, 4-dihydro-2H-pyrazino[1,2-a]indol-1-one (30 mg, 0.089 mmol, 42% Yield). ES-MS (m/z): 321.0/323.0 (M+H). ¹H NMR (400 MHz, DMSO): 12.93 (br s, 1H), 8.27 (s, 1H), 8.01 (br s, 2H), 7.74 (d, J=8.6 Hz, 1H), 7.32 (d, J=8.6 Hz, 1H), 4.81 (t, J=5.6 Hz, 2H), 3.64-3.56 (m, 2H).

### SCHEME FOR EXAMPLE 32

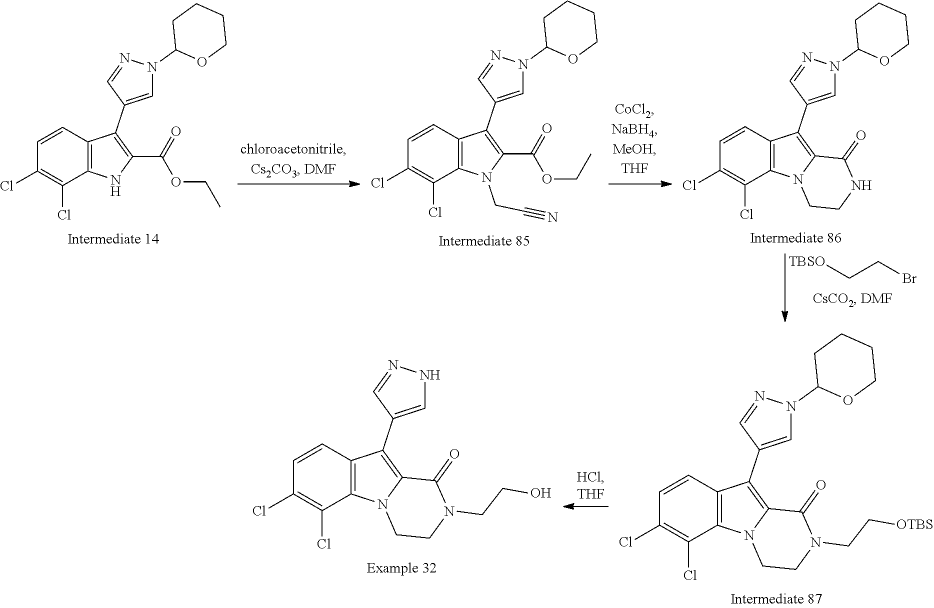 [SMILES: CCOC(=O)c1[nH]c2c(Cl)c(Cl)ccc2c1-c1cnn(C2CCCCO2)c1 ; CCOC(=O)c1c(-c2cnn(C3CCCCO3)c2)c2ccc(Cl)c(Cl)c2n1CC#N ; O=C1NCCn2c1c(-c1cnn(C3CCCCO3)c1)c1ccc(Cl)c(Cl)c12 ; CC(C)(C)[Si](C)(C)OCCBr ; O=C1c2c(-c3cn[nH]c3)c3ccc(Cl)c(Cl)c3n2CCN1CCO ; CC(C)(C)[Si](C)(C)OCCN1CCn2c(c(-c3cnn(C4CCCCO4)c3)c3ccc(Cl)c(Cl)c32)C1=O]

### Intermediate 85

### Ethyl 6, 7-dichloro-1-(cyanomethyl)-3-(1-tetrahydropyran-2-ylpyrazol-4-yl)indole-2-carboxylate

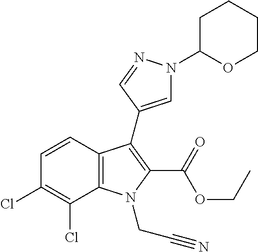 [SMILES: CCOC(=O)c1c(-c2cnn(C3CCCCO3)c2)c2ccc(Cl)c(Cl)c2n1CC#N]

Dissolve ethyl 6, 7-dichloro-3-(1-tetrahydropyran-2-ylpyrazol-4-yl)-1H-indole-2-carboxylate (1.00 g, 2.27 mmol) in DMF (10 mL). Add chloroacetonitrile (350 mg, 4.63 mmol) and Cs₂CO₃ (980 mg, 3.00 mmol). Stir 1.5 hours at RT. Dilute with water, extract with EtOAc, wash with brine, dry over anhydrous Na₂SO₄, filter and concentrate to yield ethyl 6,7-dichloro-1-(cyanomethyl)-3-(1-tetrahydropyran-2-ylpyrazol-4-yl)indole-2-carboxylate (660 mg, 1.43 mmol, 63% Yield). ES-MS (m/z): 447.0/448.8 (M+H).

### Intermediate 86

### 6, 7-Dichloro-10-(1-tetrahydropyran-2-ylpyrazol-4-yl)-3,4-dihydro-2H-pyrazino[1,2-a]indol-1-one

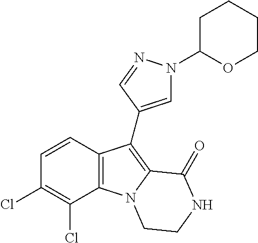 [SMILES: O=C1NCCn2c1c(-c1cnn(C3CCCCO3)c1)c1ccc(Cl)c(Cl)c12]

Suspend ethyl 6, 7-dichloro-1-(cyanomethyl)-3-(1-tetrahydropyran-2-ylpyrazol-4-yl)indole-2-carboxylate (660 mg, 1.37 mmol) and CoCl₂ (365 mg, 2.70 mmol) in MeOH (6 mL) and THF (3 mL). Add NaBH₄ (320 mg, 8.28 mmol) portion-wise at 0° C. Stir for 1 hour at 0° C. Quench with water, dilute with EtOAc, wash with brine, dry over anhydrous Na₂SO₄, filter, and concentrate. Purify by flash column chromatography (MeOH/DCM) to yield 6, 7-dichloro-10-(1-tetrahydropyran-2-ylpyrazol-4-yl)-3, 4-dihydro-2H-pyrazino[1, 2-a]indol-1-one (220 mg, 0.434 mmol, 32% Yield). ES-MS (m/z): 405.0/406.8 (M+H).

### Intermediate 87

### 2-[2-[tert-Butyl(dimethyl)silyl]oxyethyl]-6, 7-dichloro-10-(1-tetrahydropyran-2-ylpyrazol-4-yl)-3, 4-dihydropyrazino[1,2-a]indol-1-one

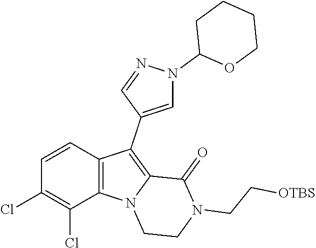 [SMILES: CC(C)(C)[Si](C)(C)OCCN1CCn2c(c(-c3cnn(C4CCCCO4)c3)c3ccc(Cl)c(Cl)c32)C1=O]

Suspend 6, 7-dichloro-10-(1-tetrahydropyran-2-ylpyrazol-4-yl)-3, 4-dihydro-2H-pyrazino[1, 2-a]indol-1-one (150 mg, 0.296 mmol) and Cs₂CO₃ (180 mg, 0.552 mmol) in DMF (2 mL). Add 2-bromoethoxy-tert-butyl-dimethyl-silane (135 mg, 0.564 mmol) and stir overnight at 60° C. Dilute with water, extract with EtOAc, wash with brine, dry over anhydrous Na₂SO₄, filter and concentrate. Purify by flash column chromatography (EtOAc/hexane) to yield 2-[2-[tert-butyl(dimethyl)silyl]oxyethyl]-6,7-dichloro-10-(1-tetrahydropyran-2-ylpyrazol-4-yl)-3,4-dihydro pyrazino[1,2-a]indol-1-one (170 mg, 0.265 mmol, 90% Yield) as colorless oil. ES-MS (m/z): 563.2/565.1 (M+H).

### EXAMPLE 32

### 6,7-Dichloro-2-(2-hydroxyethyl)-10-(1H-pyrazol-4-yl)-3,4-dihydropyrazino[1,2-a]indol-1-one

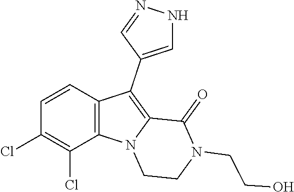 [SMILES: O=C1c2c(-c3cn[nH]c3)c3ccc(Cl)c(Cl)c3n2CCN1CCO]

Suspend 2-[2-[tert-butyl(dimethyl)silyl]oxyethyl]-6,7-dichloro-10-(1-tetrahydropyran-2-ylpyrazol-4-yl)-3, 4-dihydropyrazino[1,2-a]indol-1-one (170 mg, 0.265 mmol) in THF (2 mL) and add 6N aqueous HCl (2 mL). Stir for 1 hour at RT. Dilute with water, neutralize with saturate aqueous NaHCO₃, extract with EtOAc, wash with brine, dry over anhydrous sodium sulfate, filter, and concentrate. Purify by prep-HPLC to yield 6, 7-dichloro-2-(2-hydroxyethyl)-10-(1H-pyrazol-4-yl)-3,4-dihydro pyrazino[1,2-a]indol-1-one (56.25 mg, 0.15 mmol, 58% Yield). ES-MS (m/z): 365.0/367.0 (M+H). ¹H NMR (400 MHz, DMSO): 12.94 (br s, 1H), 8.21-7.78 (m, 2H), 7.72 (d, J=8.6 Hz, 1H), 7.31 (d, J=8.6 Hz, 1H), 4.85 (dd, J=6.4, 4.8 Hz, 2H), 4.82 (t, J=5.1 Hz, 1H), 3.84 (dd, J=6.4, 4.8 Hz, 2H), 3.62-3.53 (m, 4H).

### SCHEME FOR EXAMPLE 33

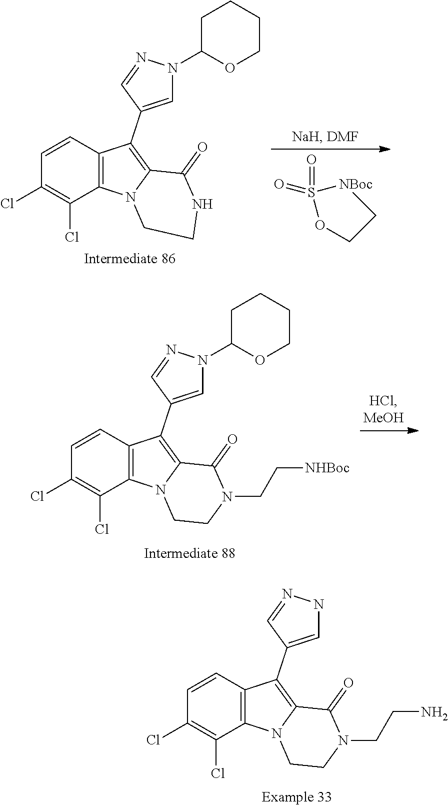 [SMILES: O=C1NCCn2c1c(-c1cnn(C3CCCCO3)c1)c1ccc(Cl)c(Cl)c12 ; CC(C)(C)OC(=O)N1CCOS1(=O)=O ; CC(C)(C)OC(=O)NCCN1CCn2c(c(-c3cnn(C4CCCCO4)c3)c3ccc(Cl)c(Cl)c32)C1=O ; NCCN1CCn2c(c(-c3cn[nH]c3)c3ccc(Cl)c(Cl)c32)C1=O]

### Intermediate 88

### tert-Butyl N-[2-[6,7-dichloro-1-oxo-10-(1-tetrahydropyran-2-ylpyrazol-4-yl)-3,4-dihydropyrazino[1,2-a]indol-2-yl]ethyl]carbamate

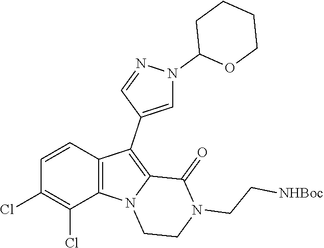 [SMILES: CC(C)(C)OC(=O)NCCN1CCn2c(c(-c3cnn(C4CCCCO4)c3)c3ccc(Cl)c(Cl)c32)C1=O]

Dissolve 6, 7-dichloro-10-(1-tetrahydropyran-2-ylpyrazol-4-yl)-3, 4-dihydro-2H-pyrazino[1,2-a] indol-1-one (130 mg, 0.304 mmol) in DMF (4 mL). Add NaH (27 mg, 0.68 mmol, 60% dispersion in mineral oil) and tert-butyl 1, 2, 3-oxathiazolidine-3-carboxylate 2, 2-dioxide (150 mg, 0.638 mmol) at 0° C. Stir overnight at RT. Quench with 1N aqueous HCl at 0° C., extract with EtOAc, wash with brine, dry over anhydrous Na₂SO₄, filter, and concentrate. Purify by flash column chromatography (EtOAc/petroleum ether) to yield tert-butyl N-[2-[6, 7-dichloro-1-oxo-10-(1-tetrahydropyran-2-ylpyrazol-4-yl)-3,4-dihydropyrazino[1,2-a]indol-2-yl]ethyl]carba mate (125 mg, 0.216 mmol, 71% Yield).

### EXAMPLE 33

### 2-(2-Aminoethyl)-6, 7-dichloro-10-(1H-pyrazol-4-yl)-3,4-dihydropyrazino[1,2-a]indol-1-one

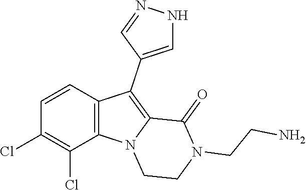 [SMILES: NCCN1CCn2c(c(-c3cn[nH]c3)c3ccc(Cl)c(Cl)c32)C1=O]

Suspend tert-butyl N-[2-[6, 7-dichloro-1-oxo-10-(1-tetrahydropyran-2-ylpyrazol-4-yl)-3, 4-dihydropyrazino[1,2-a]indol-2-yl]ethyl]carbamate (175 mg, 0.303 mmol) in 4N HCl/MeOH (4 mL) and stir 1 hour at RT. Concentrate and dilute with EtOAc and water, adjust to pH 8 with saturated aqueous NaHCO₃, extract with EtOAc, wash with brine, dry over anhydrous Na₂SO₄, filter and concentrate. Purify by prep-HPLC to yield 2-(2-aminoethyl)-6, 7-dichloro-10-(1H-pyrazol-4-yl)-3, 4-dihydropyrazino[1,2-a]indol-1-one (51 mg, 0.14 mmol, 46% Yield, HCl salt). ES-MS (m/z): 364.0/366.0 (M+H). ¹H NMR (400 MHz, DMSO): 8.10 (s, 2H), 8.07 (br s, 3H), 7.74 (d, J=8.6 Hz, 1H), 7.33 (d, J=8.6 Hz, 1H), 4.92 (dd, J=6.4, 4.8 Hz, 2H), 3.86 (dd, J=6.4, 4.8 Hz, 2H), 3.75 (t, J=5.8 Hz, 2H), 3.11-3.02 (m, 2H).

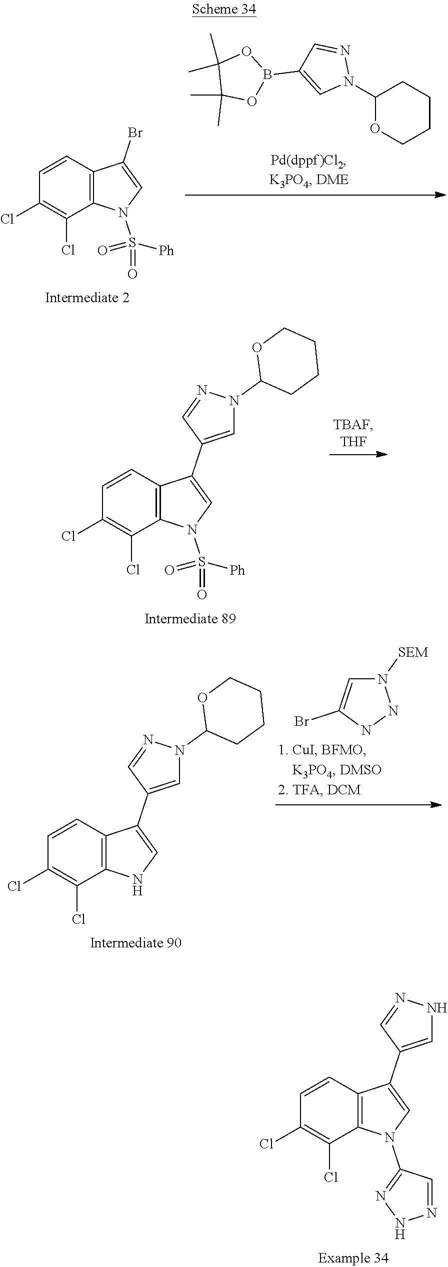

### Intermediate 89

### 1-(Benzenesulfonyl)-6,7-dichloro-3-(1-tetrahydropyran-2-ylpyrazol-4-yl)indole

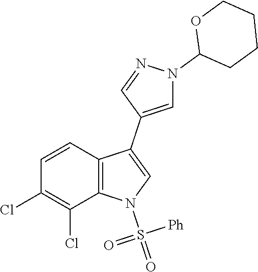 [SMILES: O=S(=O)(c1ccccc1)n1cc(-c2cnn(C3CCCCO3)c2)c2ccc(Cl)c(Cl)c21]

Combine 1-(benzenesulfonyl)-3-bromo-6,7-dichloro-indole (3.30 g, 8.15 mmol), 1-tetrahydropyran-2-yl-4-(4,4,5,5-tetramethyl-1,3,2-dioxaborolan-2-yl)pyrazole (4.54 g, 16.3 mmol), Pd(dppf)Cl₂ (2.50 g, 3.25 mmol) in DME (55.0 mL, 530 mmol, 100 mass %). Add aqueous K₃PO₄ (24.0 mL, 24.0 mmol, 1 mol/L), sparge with nitrogen and heat to 80° C. Concentrate and add EtOAc and water. Filter through diatomaceous earth into a separatory funnel and extract with EtOAc. Wash the organic layer with water (3×) and brine, dry over anhydrous magnesium sulfate, filter, and concentrate. Purify by flash chromatography (EtOAc/hexanes) to yield 1-(benzenesulfonyl)-6,7-dichloro-3-(1-tetrahydropyran-2-ylpyrazol-4-yl)indole (2.88 g, 6.05 mmol, 74%). ES-MS (m/z): 476.0/478.0 (M+H).

### Intermediate 90

### 6,7-Dichloro-3-(1-tetrahydropyran-2-ylpyrazol-4-yl)-1H-indole

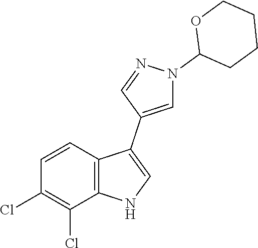 [SMILES: Clc1ccc2c(-c3cnn(C4CCCCO4)c3)c[nH]c2c1Cl]

Dissolve 1-(benzenesulfonyl)-6,7-dichloro-3-(1-tetrahydropyran-2-ylpyrazol-4-yl)indole (2.88 g, 6.05 mmol) in 2-methyltetrahydrofuran (60.0 mL) and add TBAF (30.0 mL, 30.0 mmol, 1 mol/L in THF) at RT. Stir 2 hours. Dilute with EtOAc and wash with saturated aqueous sodium bicarbonate (2×) and brine, dry over anhydrous magnesium sulfate, filter and concentrate. Purify by flash chromatography to yield 6,7-dichloro-3-(1-tetrahydropyran-2-ylpyrazol-4-yl)-1H-indole (1.80 g, 5.35 mmol, 89%). ES-MS (m/z): 335.8/337.8 (M+H).

### EXAMPLE 34

### 6,7-Dichloro-3-(1H-pyrazol-4-yl)-1-(2H-triazol-4-yl)indole

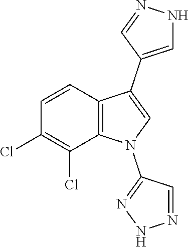 [SMILES: Clc1ccc2c(-c3cn[nH]c3)cn(-c3cn[nH]n3)c2c1Cl]

Suspend 6,7-dichloro-3-(1-tetrahydropyran-2-ylpyrazol-4-yl)-1H-indole (310 mg, 0.784 mmol), a mixture of 1-[(4-bromotriazol-2-yl)methoxy]ethyl-trimethyl-silane and 2-[(4-bromotriazol-2-yl)methoxy]ethyl-trimethyl-silane (333 mg, 1.17 mmol), CuI (24 mg, 0.16 mmol), BFMO (41 mg, 0.16 mmol) and K₃PO₄ (338 mg, 1.56 mmol) in DMSO (6 mL). Sparge with nitrogen and heat to 100° C. overnight. Cool to room temperature. Dilute with water, extract with DCM, wash with brine, dry over anhydrous sodium sulfate, filter, and concentrate. Purify by flash column chromatography (EtOAc/PE) to yield a mixture of 1-[[4-[6,7-dichloro-3-(1-tetrahydropyran-2-ylpyrazol-4-yl)indol-1-yl]triazol-2-yl]methoxy]ethyl-trimethyl-silane and 2-[[4-[6,7-dichloro-3-(1-tetrahydropyran-2-ylpyrazol-4-yl)indol-1-yl]triazol-2-yl]methoxy]ethyl-trimethyl-silane. Dissolve in DCM (3 mL). Add TFA (3 mL) and stir for 3 hours at RT. Concentrate and dilute with EtOAc and water, adjust to pH 8 by saturated aqueous sodium bicarbonate. Extract with EtOAc, wash with brine, dry over anhydrous sodium sulfate, filter, and concentrate. Purify by prep-HPLC to yield 6,7-dichloro-3-(1H-pyrazol-4-yl)-1-(2H-triazol-4-yl)indole (Product, 9.8 mg, 0.030 mmol, 21%). ES-MS (m/z): 319.2/321.2 (M+H). ¹H NMR (400 MHz, DMSO): 13.01 (br s, 1H), 8.30 (br s, 1H), 8.23 (br s, 1H), 7.93 (br s, 1H), 7.93 (d, J=8.5 Hz, 1H), 7.80 (s, 1H), 7.41 (d, J=8.5 Hz, 1H).

### SCHEME FOR EXAMPLE 35

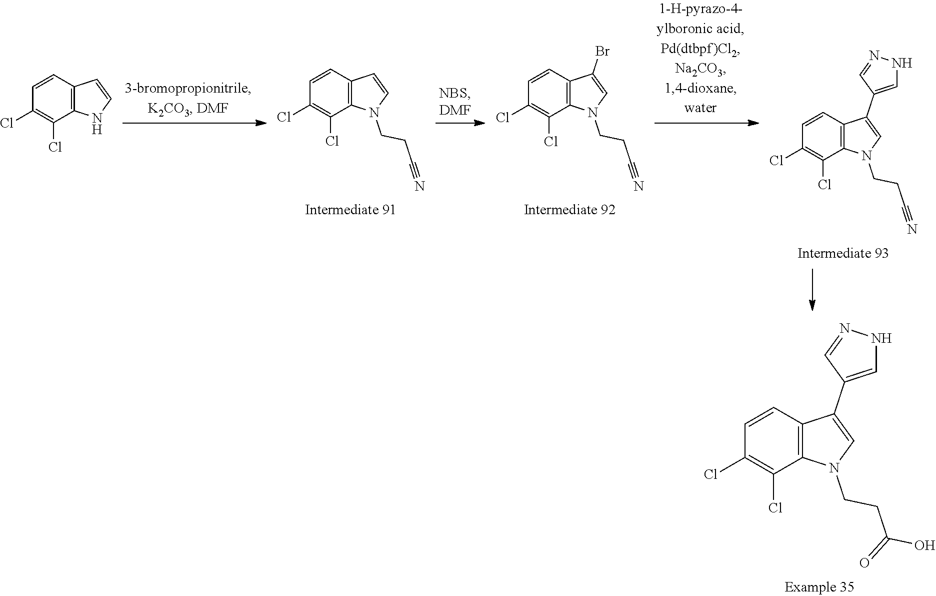 [SMILES: Clc1ccc2cc[nH]c2c1Cl ; N#CCCn1ccc2ccc(Cl)c(Cl)c21 ; N#CCCn1cc(Br)c2ccc(Cl)c(Cl)c21 ; N#CCCn1cc(-c2cn[nH]c2)c2ccc(Cl)c(Cl)c21 ; O=C(O)CCn1cc(-c2cn[nH]c2)c2ccc(Cl)c(Cl)c21]

### Intermediate 91

### 3-(6, 7-Dichloro-1H-indol-1-yl)propanenitrile

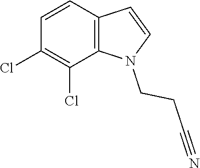 [SMILES: N#CCCn1ccc2ccc(Cl)c(Cl)c21]

Dissolve 6, 7-dichloro-1H-indole (1.20 g, 5.80 mmol) and 3-bromopropionitrile (1.20 g, 9.00 mmol) in DMF (20.0 mL). Add K₂CO₃ (1.21 g, 8.58 mmol) at room temperature. Stir for 12 hours at 60° C. Filter to remove K₂CO₃, then add water, extract with EtOAc, dry over anhydrous sodium sulfate, filter, and concentrate. Purify by flash column chromatography (EtOAc/petroleum ether) to yield 3-(6, 7-dichloro-1H-indol-1-yl)propanenitrile (530 mg, 2.17 mmol, 37% Yield).

### Intermediate 92

### 3-(3-Bromo-6, 7-dichloro-1H-indol-1-yl)propanenitrile

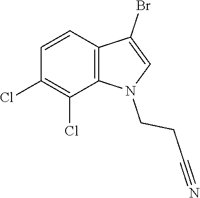 [SMILES: N#CCCn1cc(Br)c2ccc(Cl)c(Cl)c21]

Dissolve 3-(6, 7-dichloro-1H-indol-1-yl)propanenitrile (530 mg, 2.17 mmol) in DMF (15 mL) and add NBS (425 mg, 2.39 mmol). Stir for 2 hours at RT. Quench with water and stir vigorously for 15 minutes. Extract with EtOAc, wash with brine, dry over sodium sulfate, filter, and concentrate. Purify by flash column chromatography (EtOAc/petroleum ether) to yield 3-(3-bromo-6, 7-dichloro-indol-1-yl)propanenitrile (624 mg, 1.96 mmol, 90% Yield).

### Intermediate 93

### 3-[6, 7-Dichloro-3-(1H-pyrazol-4-yl)indol-1-yl]propanenitrile

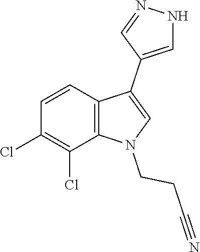 [SMILES: N#CCCn1cc(-c2cn[nH]c2)c2ccc(Cl)c(Cl)c21]

Suspend 3-(3-bromo-6, 7-dichloro-indol-1-yl)propanenitrile (620 mg, 1.95 mmol), 1H-pyrazol-4-ylboronic acid (322 mg, 2.88 mmol), Na₂CO₃ (413 mg, 3.82 mmol), Pd(dtbpf)Cl₂ (128 mg, 0.190 mmol) in 1,4-dioxane (15 mL) and water (2 mL). Sparge with nitrogen and heat to 100° C. for 16 hours. Cool to RT, dilute with EtOAc, wash sequentially with saturated aqueous sodium bicarbonate and brine, dry over sodium sulfate, filter, and concentrate. Purified by flash silica gel chromatography (EtOAc/petroleum ether) to yield 3-[6, 7-dichloro-3-(1H-pyrazol-4-yl)indol-1-yl]propanenitrile (110 mg, 0.320 mmol, 16% Yield). ES-MS (m/z): 305.1/307.1 (M+H)

### EXAMPLE 35

### 3-[6, 7-Dichloro-3-(1H-pyrazol-4-yl)indol-1-yl]propanoic acid

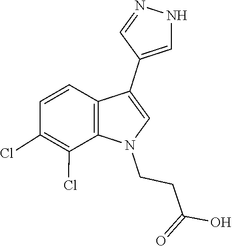 [SMILES: O=C(O)CCn1cc(-c2cn[nH]c2)c2ccc(Cl)c(Cl)c21]

Dissolve 3-[6, 7-dichloro-3-(1H-pyrazol-4-yl)indol-1-yl]propanenitrile (70 mg, 0.20 mmol) in concentrated HCl (3 mL) and stir at 100° C. for 16 hours. Cool to RT, add formic acid (2 mL) and filter the precipitate. Trituration with ACN yields 3-[6, 7-dichloro-3-(1H-pyrazol-4-yl)indol-1-yl]propanoic acid (Product, 32 mg, 0.098 mmol, 48% Yield). ES-MS (m/z): 324.0/326.0 (M+H). ¹H NMR (400 MHz, DMSO): 8.07 (s, 2H), 7.78 (d, J=8.5 Hz, 1H), 7.70 (s, 1H), 7.28 (d, J=8.5 Hz, 1H), 4.74 (t, J=7.1 Hz, 2H), 2.81 (t, J=7.1 Hz, 2H).

### SCHEME FOR EXAMPLE 36

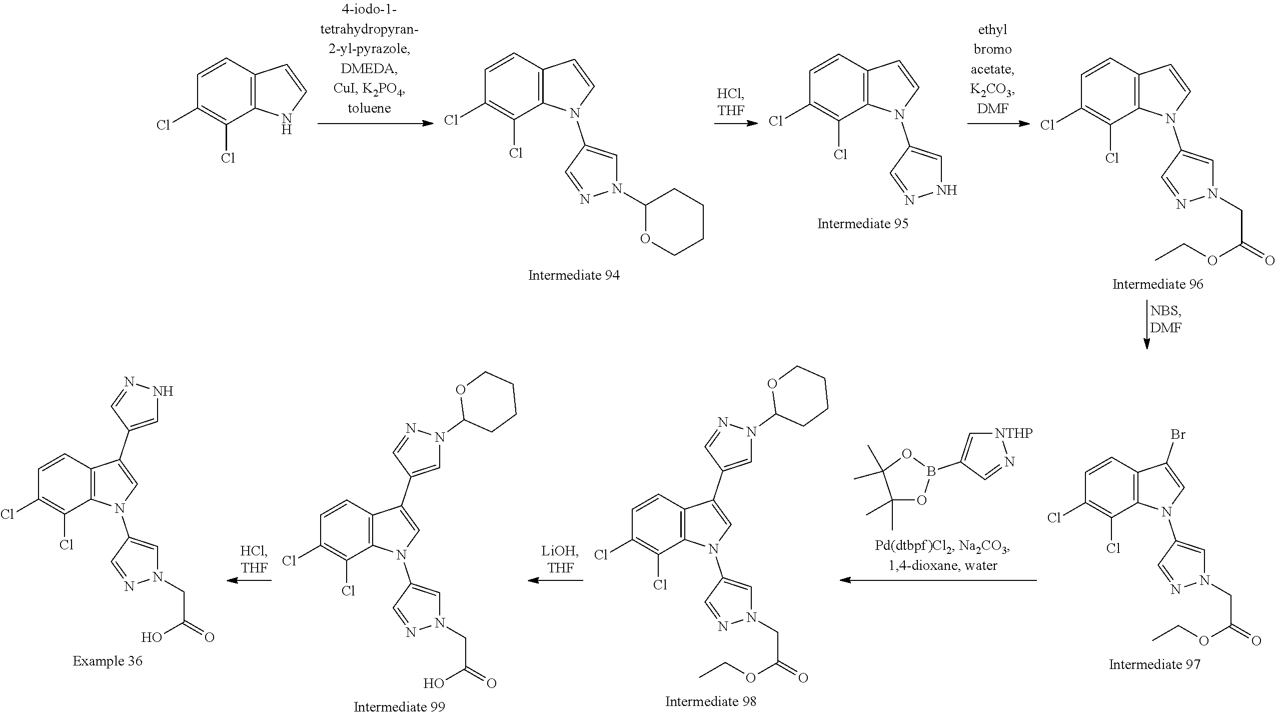

### Intermediate 94

### 6,7-Dichloro-1-(1-tetrahydropyran-2-ylpyrazol-4-yl)indole

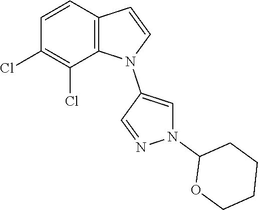 [SMILES: Clc1ccc2ccn(-c3cnn(C4CCCCO4)c3)c2c1Cl]

Suspend 6,7-dichloro-1H-indole (1.00 g, 4.84 mmol), 4-iodo-1-tetrahydropyran-2-yl-pyrazole (2.00 g, 7.19 mmol), CuI (90 mg, 0.47 mmol), DMEDA (175 mg, 1.95 mmol) and K₃PO₄ (3.14 g, 14.5 mmol) in toluene (10 mL). Sparge with nitrogen and heat to 140° C. for 3 hours in the microwave. Cool to RT and filter. Add water to the filtrate, extract with EtOAc, wash with brine, dry over sodium sulfate, filter, and concentrate. Purify by flash column chromatography (PE/EtOAc) to yield 6,7-dichloro-1-(1-tetrahydropyran-2-ylpyrazol-4-yl)indole (2.00 g, 3.57 mmol, 74% Yield). ES-MS (m/z): 336.2/338.2 (M+H).

### Intermediate 95

### 6,7-Dichloro-1-(1H-pyrazol-4-yl)indole

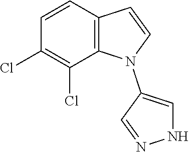 [SMILES: Clc1ccc2ccn(-c3cn[nH]c3)c2c1Cl]

Dissolve 6,7-dichloro-1-(1-tetrahydropyran-2-ylpyrazol-4-yl)indole (1.90 g, 3.40 mmol) in THF (3 mL) and add 4 N aqueous HCl (10 mL). Stir at 25° C. overnight. Dilute with water, extract with EtOAc, wash with brine, dry over sodium sulfate, filter, and concentrate. Purify by flash column chromatography (PE/EtOAc) to yield 6,7-dichloro-1-(1H-pyrazol-4-yl)indole (700 mg, 2.50 mmol, 74% Yield). ES-MS (m/z): 251.9/253.9 (M+H).

### Intermediate 96

### Ethyl 2-[4-(6,7-dichloroindol-1-yl)pyrazol-1-yl]acetate

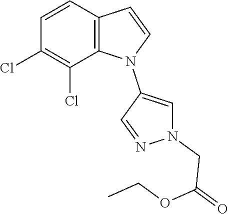 [SMILES: CCOC(=O)Cn1cc(-n2ccc3ccc(Cl)c(Cl)c32)cn1]

Dissolve 6,7-dichloro-1-(1H-pyrazol-4-yl)indole (700 mg, 2.50 mmol), ethyl bromoacetate (668 mg, 0.5 mL, 4.00 mmol) and K₂CO₃ (700 mg, 5.06 mmol) in DMF (5 mL). Stir at 50° C. for 3 hours. Cool to RT and filter. Add water to the filtrate, extract with EtOAc, wash with brine, dry over sodium sulfate, filter, and concentrate. Purify by flash column chromatography (PE/EtOAc) to yield ethyl 2-[4-(6,7-dichloroindol-1-yl)pyrazol-1-yl]acetate (600 mg, 1.42 mmol, 57% Yield).

### Intermediate 97

### Ethyl 2-[4-(3-bromo-6,7-dichloro-indol-1-yl)pyrazol-1-yl]acetate

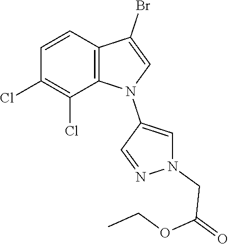 [SMILES: CCOC(=O)Cn1cc(-n2cc(Br)c3ccc(Cl)c(Cl)c32)cn1]

Dissolve ethyl 2-[4-(6,7-dichloroindol-1-yl)pyrazol-1-yl]acetate (500 mg, 1.33 mmol) in DMF (5 mL) and add NBS (350 mg, 1.93 mmol). Stir at 20° C. overnight. Dilute with water, extract with EtOAc, wash with brine, dry over sodium sulfate, filter, and concentrate. Purify by flash column chromatography (PE/EtOAc) to yield ethyl 2-[4-(3-bromo-6,7-dichloro-indol-1-yl)pyrazol-1-yl]acetate (140 mg, 0.302 mmol, 23% Yield). ES-MS (m/z): 415.9/417.9/420.0 (M+H).

### Intermediate 98

### Ethyl 2-[4-[6,7-dichloro-3-(1-tetrahydropyran-2-ylpyrazol -4-yl)indol -1-yl]pyrazol -1-yl]acetate

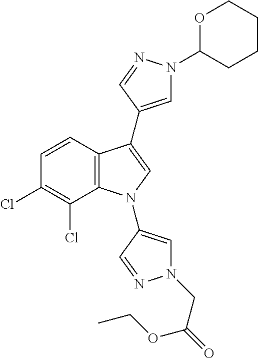 [SMILES: CCOC(=O)Cn1cc(-n2cc(-c3cnn(C4CCCCO4)c3)c3ccc(Cl)c(Cl)c32)cn1]

Suspend ethyl 2-[4-(3-bromo-6,7-dichloro-indol -1-yl)pyrazol -1-yl]acetate (120 mg, 0.230 mmol), (tetrahydro-2h-pyran-2-yl)-4-(4,4,5,5-tetramethyl -1,3,2-dioxaborolan-2-yl)-1h-pyrazole (120 mg, 0.410 mmol), Pd(dtbpf)Cl₂ (34 mg, 0.051 mmol) and Na₂CO₃ (80 mg, 0.75 mmol) in dioxane (3 mL) and water (0.5 mL). Sparge with nitrogen and heat to 90° C. for 1 hour. Dilute with water, extract with EtOAc, wash with brine, dry over sodium sulfate, filter, and concentrate. Purify by flash column chromatography (PE/EtOAc) to yield ethyl 2-[4-[6,7-dichloro-3-(1-tetrahydropyran-2-ylpyrazol-4-yl)indol-1-yl]pyrazol-1-yl]acetate (45 mg, 0.077 mmol, 34% Yield). ES-MS (m/z): 488.1/490.1 (M+H).

### Intermediate 99

### 2-[4-[6,7-Dichloro-3-(1-tetrahydropyran-2-ylpyrazol-4-yl)indol-1-yl]pyrazol-1-yl]acetic acid

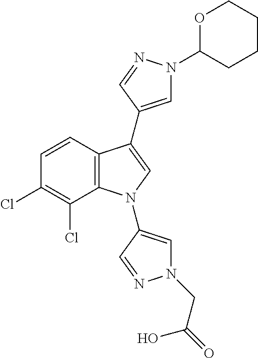 [SMILES: O=C(O)Cn1cc(-n2cc(-c3cnn(C4CCCCO4)c3)c3ccc(Cl)c(Cl)c32)cn1]

Dissolve ethyl 2-[4-[6,7-dichloro-3-(1-tetrahydropyran-2-ylpyrazol-4-yl)indol-1-yl]pyrazol-1-yl]acetate (45 mg, 0.077 mmol) in THF (2 mL) and water (0.5 mL). Add LiOH·H₂O (20 mg, 0.82 mmol). Stir at 40° C. overnight. Dilute with water and adjust to pH 2 with 1 N aqueous HCl. Extract with EtOAc, wash with brine, dry over sodium sulfate, filter, and concentrate to yield 2-[4-[6,7-dichloro-3-(1-tetrahydropyran-2-ylpyrazol-4-yl)indol-1-yl]pyrazol-1-yl]acetic acid (30 mg, 0.045 mmol, 59% Yield). ES-MS (m/z): 460.1/462.1 (M+H).

### EXAMPLE 36

### 2-[4-[6,7-Dichloro-3-(1H-pyrazol-4-yl)indol-1-yl]pyrazol-1-yl]acetic acid

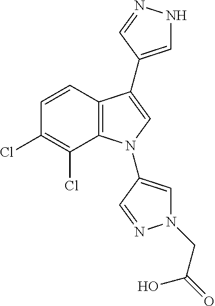 [SMILES: O=C(O)Cn1cc(-n2cc(-c3cn[nH]c3)c3ccc(Cl)c(Cl)c32)cn1]

Dissolve 2-[4-[6,7-dichloro-3-(1-tetrahydropyran-2-ylpyrazol-4-yl)indol-1-yl]pyrazol-1-yl]acetic acid (60 mg, 0.091 mmol) in THF (1 mL) and add 4 N aqueous HCl (3 mL). Stir at 25° C. for 4 hours. Dilute with water, extract with EtOAc, wash with brine, dry over sodium sulfate, filter, and concentrate. Purify by prep-HPLC to yield 2-[4-[6,7-dichloro-3-(1H-pyrazol-4-yl)indol-1-yl]pyrazol-1-yl]acetic acid (8.6 mg, 0.022 mmol, 24% Yield). ES-MS (m/z): 376.2/378.2 (M+H). ¹H NMR (400 MHz, DMSO): 13.01 (br s, 1H), 8.13 (s, 1H), 8.06 (br s, 2H), 7.88 (d, J=8.5 Hz, 1H), 7.72 (s, 1H), 7.67 (s, 1H), 7.35 (d, J=8.5 Hz, 1H), 4.97 (s, 2H).

### SCHEME FOR EXAMPLE 37

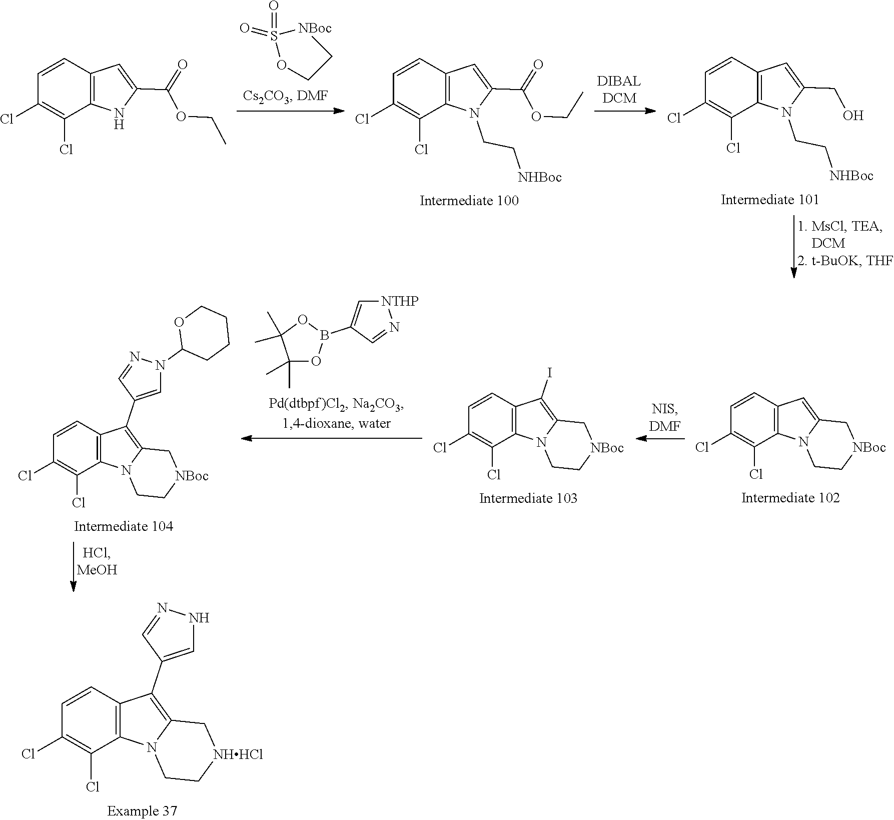

### Intermediate 100

### Ethyl 1-[2-(tert-butoxycarbonylamino)ethyl]-6,7-dichloro-indole-2-carboxylate

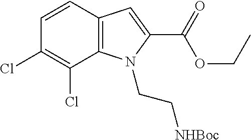 [SMILES: CCOC(=O)c1cc2ccc(Cl)c(Cl)c2n1CCNC(=O)OC(C)(C)C]

Suspend ethyl 6, 7-dichloro-1H-indole-2-carboxylate (10.0 g, 36.8 mmol) and Cs₂CO₃ (24.0 g, 73.6 mmol) in DMF (100 mL) and add tert-butyl 1, 2, 3-oxathiazolidine-3-carboxylate 2,2-dioxide (13.0 g, 55.3 mmol). Stir overnight at RT. Quench with 1 N aqueous HCl at 0° C. and extract with EtOAc, wash with brine, dry over anhydrous Na₂SO₄, filter and concentrate. Purify by flash column chromatography (EtOAc/petroleum ether) to yield ethyl 1-[2-(tert-butoxycarbonylamino) ethyl]-6, 7-dichloro-indole-2-carboxylate (12.6 g, 29.8 mmol, 81% Yield).

### Intermediate 101

### tert-Butyl (2-(6,7-dichloro-2-(hydroxymethyl)-1H-indol-1-yl)ethyl)carbamate

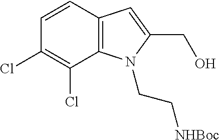 [SMILES: CC(C)(C)OC(=O)NCCn1c(CO)cc2ccc(Cl)c(Cl)c21]

Dissolve ethyl 1-[2-(tert-butoxycarbonylamino)ethyl]-6, 7-dichloro-indole-2-carboxylate (15.0 g, 59.7 mmol) in DCM (150 mL). Add DIBAL dropwise (140 mL, 140 mmol, 1M in toluene) at −78° C. under N₂ atmosphere. Stir for 1 hour at RT. Dilute with DCM, quench with saturated aqueous potassium sodium tartrate and stir overnight at RT. Extract with DCM, wash with brine, dry over anhydrous Na₂SO₄, filter and concentrate. Trituate (ethyl acetate/petroleum ether=10:1) to yield tert-butyl (2-(6, 7-dichloro-2-(hydroxymethyl)-1H-indol-1-yl)ethyl)carbamate (9.45 g, 25.0 mmol, 71% Yield).

### Intermediate 102

### tert-Butyl 6,7-dichloro-3,4-dihydropyrazino[1,2-a]indole-2(1H)-carboxylate

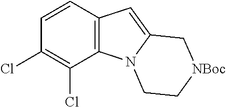 [SMILES: CC(C)(C)OC(=O)N1CCn2c(cc3ccc(Cl)c(Cl)c32)C1]

Dissolve tert-butyl (2-(6, 7-dichloro-2-(hydroxymethyl)-1H-indol-1-yl)ethyl)carbamate (27.0 g, 63.8 mmol) in DCM (250 mL). Add Et₃N (29.0 mL, 208 mmol) and MsCl (15.9 g, 137 mmol) at ° C. Stir overnight at RT. Quench with saturated aqueous NaHCO₃ and extract with DCM, wash with brine, dry over anhydrous Na₂SO₄, filter and concentrate to yield (1-(2-((tert-butoxycarbonyl)amino)ethyl)-6,7-dichloro-1H-indol-2-yl)methyl methanesulfonate. Resuspend in THF (250 mL), add t-BuOK (13.5 g, 119 mmol). Stir and heat at 60° C. for 2 hours. Cool to RT, quench with water, extract with EtOAc, wash with brine, dry over anhydrous Na₂SO₄, filter and concentrate. Purify by flash column chromatography (EtOAc/petroleum ether) to yield tert-butyl 6, 7-dichloro-3,4-dihydropyrazino[1, 2-a]indole-2(1H)-carboxylate (14.6 g, 41.9 mmol, 72% Yield).

### Intermediate 103

### tert-Butyl 6,7-dichloro-10-iodo-3,4-dihydropyrazino[1,2-a]indole-2(1H)-carboxylate

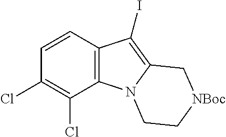 [SMILES: CC(C)(C)OC(=O)N1CCn2c(c(I)c3ccc(Cl)c(Cl)c32)C1]

Dissolve tert-butyl 6, 7-dichloro-3, 4-dihydropyrazino[1,2-a]indole-2(1H)-carboxylate (13.0 g, 36.2 mmol) in DMF (130 mL) and add NIS (12.7 mg, 54.8 mmol) at 0° C. Stir 1 hour at RT. Quench with saturated aqueous Na₂SO₃ and extract with EtOAc. Wash with brine, dry over anhydrous Na₂SO₄, filter and concentrate. Purify by flash column chromatography (EtOAc/petroleum ether) to yield tert-butyl 6, 7-dichloro-10-iodo-3, 4-dihydropyrazino[1,2-a]indole-2(1H)-carboxylate (15.0 g, 28.9 mmol, 80% Yield).

### Intermediate 104

### tert-Butyl 6,7-dichloro-10-(1-(tetrahydro-2H-pyran-2-yl)-1H-pyrazol-4-yl)-3,4-dihydropyrazino[1,2-a]indole-2(1H)-carboxylate

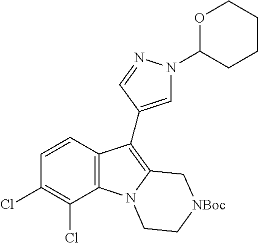 [SMILES: CC(C)(C)OC(=O)N1CCn2c(c(-c3cnn(C4CCCCO4)c3)c3ccc(Cl)c(Cl)c32)C1]

Suspend tert-butyl 6, 7-dichloro-10-iodo-3, 4-dihydropyrazino[1, 2-a]indole-2(1H)-carboxylate (10.0 g, 19.2 mmol), 1-(tetrahydro-2H-pyran-2-yl)-4-(4,4,5,5-tetramethyl-1,3,2-dioxaborolan-2-yl)-1H-pyrazole (8.10 g, 29.0 mmol), Pd(dtbpf)Cl₂ (1.30 g, 2.00 mmol) and Na₂CO₃ (6.30 g, 59.0 mmol) in 1,4-dioxane (80 mL) and water (20 mL). Sparge with nitrogen and heat at 90° C. for 1 hour. Cool to RT, dilute with water, extract with EtOAc, wash with brine, dry over anhydrous Na₂SO₄, filter and concentrate. Purify by flash column chromatography (EtOAc/petroleum ether) to yield tert-butyl 6, 7-dichloro-10-(1-(tetrahydro-2H-pyran-2-yl)-1H-pyrazol-4-yl)-3, 4-dihydropyrazino[1,2-a]indole-2(1H)-carboxylate (8.75 g, 16.9 mmol, 88% Yield).

### EXAMPLE 37

### 6, 7-Dichloro-10-(1H-pyrazol-4-yl)-1, 2, 3, 4-tetrahydropyrazino[1,2-a]indole hydrochloride

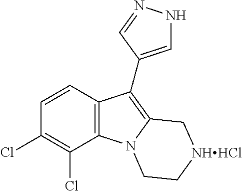

Dissolve tert-butyl 6, 7-dichloro-10-(1-(tetrahydro-2H-pyran-2-yl)-1H-pyrazol-4-yl)-3,4-dihydro pyrazino[1,2-a]indole-2(1H)-carboxylate (13.5 g, 26.9 mmol) in 4 N HCl/MeOH (60 mL) and stir 1 hour at RT. Concentrate and triturate with MeOH. Collect solid by suction filtration and dry to yield 6, 7-dichloro-10-(1H-pyrazol-4-yl)-1, 2, 3, 4-tetrahydropyrazino[1,2-a]indole hydrochloride (8.50 g, 24.0 mmol, 90% Yield, HCl salt). ES-MS (m/z): 307.0/309.0 (M+H). ¹H NMR (400 MHz, DMSO): 10.08 (br s, 2H), 7.96 (s, 2H), 7.68 (d, J=8.5 Hz, 1H), 7.33 (d, J=8.5 Hz, 1H), 4.86 (t, J=5.7 Hz, 2H), 4.62-4.54 (m, 2H), 3.70-3.61 (m, 2H).

### SCHEME FOR EXAMPLE 38

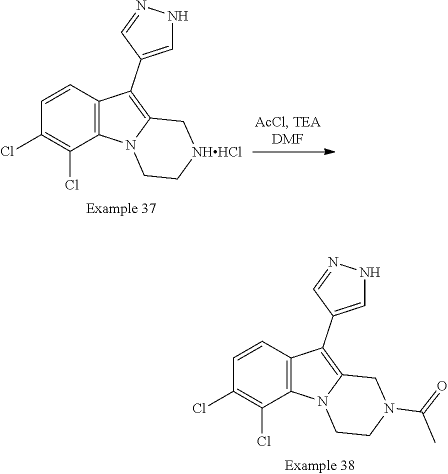

### EXAMPLE 38

### 1-[6, 7-Dichloro-10-(1H-pyrazol-4-yl)-3, 4-dihydro-1H-pyrazino[1,2-a]indol-2-yl]ethanone

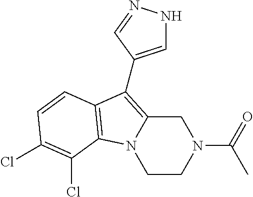 [SMILES: CC(=O)N1CCn2c(c(-c3cn[nH]c3)c3ccc(Cl)c(Cl)c32)C1]

Dissolve 6, 7-dichloro-10-(1H-pyrazol-4-yl)-1, 2, 3, 4-tetrahydropyrazino[1,2-a]indole (75 mg, 0.23 mmol, HCl salt) in DMF (3 mL). Add TEA (0.10 mL, 0.72 mmol) and acetyl chloride (0.030 mL, 0.41 mmol) at 0° C. Stir 2 hours at RT. Quench with saturated aqueous NaHCO₃ and extract with EtOAc, wash with brine 3 times, dry over anhydrous Na₂SO₄, filter, and concentrate. Purify by prep-HPLC to yield 1-[6, 7-dichloro-10-(1H-pyrazol-4-yl)-3, 4-dihydro-1H-pyrazino[1,2-a]indol-2-yl]ethanone (30.03 mg, 0.086 mmol, 37% Yield) as an off-white solid. ES-MS (m/z): 349.0/351.0 (M+H). ¹H NMR (400 MHz, DMSO): 13.09 (br s, 1H), 8.19-7.57 (m, 1H), 7.65 (d, J=8.5 Hz, 0.3H, minor rotamer), 7.63 (d, J=8.5 Hz, 0.7H, major rotamer), 7.27 (d, J=8.5 Hz, 1H), 4.96 (s, 0.6H, minor rotamer), 4.88 (s, 1.4H, major rotamer), 4.74 (t, J=5.6 Hz, 1.4H, major rotamer), 4.64 (t, J=5.6 Hz, 0.6H, minor rotamer), 3.98 (t, J=5.6 Hz, 1.4H, major rotamer), 3.93 (t, J=5.6 Hz, 0.6H, minor rotamer), 2.13 (s, 2.1H, major rotamer), 2.12 (s, 0.9H, minor rotamer).

Examples 39-53 (Table 2) were prepared by similar means from Example 38 using an appropriate electrophile in DMF or DCM in the presence of a base (eg. TEA, DIPEA). Carboxylic acids were activated using HATU.

TABLE 2

| Example | Chemical Name | Structure | Analytical Data |
|---|---|---|---|
| 39 | 6,7-dichloro-N- methyl-10-(1H- pyrazol-4-yl)-3, 4-dihydro-1H- pyrazino[1,2- a]indole-2- carboxamide | 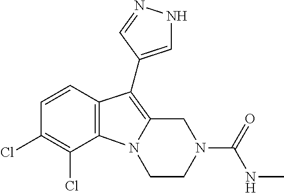 [SMILES: CNC(=O)N1CCn2c(c(-c3cn[nH]c3)c3ccc(Cl)c(Cl)c32)C1] | ES-MS (m/z): 364.0/366.0 (M + H) ¹H NMR (400 MHz, DMSO): 13.09 (br s, 1H), 8.02 (br s, 1H), 7.79 (br s, 1H), 7.64 (d, J = 8.5 Hz, 1H), 7.26 (d, J = 8.5 Hz, 1H), 6.82 (br q, J = 4.3 Hz, 1H), 4.78 (s, 2H), 4.63 (t, J = 5.4 Hz, 2H), 3.82 (t, J = 5.4 Hz, 2H), 2.60 (d, J = 4.3 Hz, 3H). |
| 40 | 6,7-dichloro-10- (1H-pyrazol-4- yl)-3, 4-dihydro- 1H-pyrazino[1,2- a]indole-2- sulfonamide | 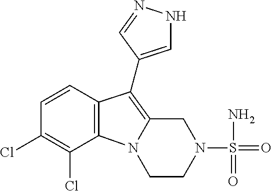 [SMILES: NS(=O)(=O)N1CCn2c(c(-c3cn[nH]c3)c3ccc(Cl)c(Cl)c32)C1] | ES-MS (m/z): 386.2/388.2 (M + H) ¹H NMR (400 MHz, DMSO): 13.14 (br s, 1H), 7.99 (br s, 1H), 7.73 (br s, 1H), 7.63 (d, J = 8.5 Hz, 1H), 7.28 (d, J = 8.5 Hz, 1H), 7.21 (s, 2H), 4.74 (t, J = 5.5 Hz, 2H), 4.43 (s, 2H), 3.55 (t, J = 5.5 Hz, 2H). |
| 41 | 6,7-dichloro-2- methylsulfonyl- 10-(1H-pyrazol-4- yl)-3,4-dihydro- 1H-pyrazino[1,2- a]indole | 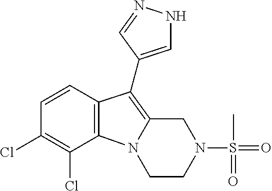 [SMILES: CS(=O)(=O)N1CCn2c(c(-c3cn[nH]c3)c3ccc(Cl)c(Cl)c32)C1] | ES-MS (m/z): 385.2/387.2 (M + H) ¹H NMR (400 MHz, DMSO): 13.14 (br s, 1H), 8.04 (br s, 1H), 7.78 (br s, 1H), 7.65 (d, J = 8.5 Hz, 1H), 7.30 (d, J = 8.5 Hz, 1H), 4.74 (t, J = 5.6 Hz, 2H), 4.61 (s, 2H), 3.73 (t, J = 5.6 Hz, 2H), 3.10 (s, 3H). |
| 42 | 1-[6,7-dichloro- 10-(1H-pyrazol-4- yl)-3, 4-dihydro- 1H-pyrazino[1,2- a]indol-2-yl]-2- methoxy- ethanone | 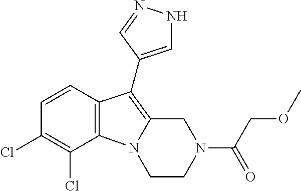 [SMILES: COCC(=O)N1CCn2c(c(-c3cn[nH]c3)c3ccc(Cl)c(Cl)c32)C1] | ES-MS (m/z): 379.0/381.0 (M + H) ¹H NMR (400 MHz, DMSO): 13.11 (br s, 1H), 8.03 (br s, 1H), 7.84-7.70 (m, 1H), 7.64 (d, J = 8.5 Hz, 1H), 7.28 (d, J = 8.5 Hz, 1H), 4.96-4.86 (m, 2H), 4.77-4.62 (m, 2H), 4.23 (s, 2H), 3.99-3.91 (m, 2H), 3.32-3.24 (m, 3H). |
| 43 | 1-[6,7-dichloro- 10-(1H-pyrazol-4- yl)-3, 4-dihydro- 1H-pyrazino[1,2- a]indol-2-yl]-2-(1, 2,4-triazol-1- yl)ethanone | 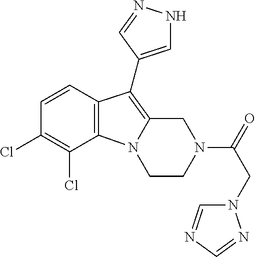 [SMILES: O=C(Cn1cncn1)N1CCn2c(c(-c3cn[nH]c3)c3ccc(Cl)c(Cl)c32)C1] | ES-MS (m/z): 416.0/418.0 (M + H) ¹H NMR (400 MHz, DMSO): 8.43 (s, 1H), 7.99 (s, 0.7H, minor rotamer), 7.96 (s, 1H), 7.87 (s, 1.3H, major rotamer), 7.69 (d, J = 8.5 Hz, 0.3H, minor rotamer), 7.64 (d, J = 8.5 Hz, 0.7H, major rotamer), 7.29 (d, J = 8.5 Hz, 1H), 5.50 (s, 0.7H, minor rotamer), 5.46 (s, 1.3H, major rotamer), 5.08 (s, 0.7H, minor rotamer), 4.92 (s, 1.3H, major rotamer), 4.82 (t, J = 5.4 Hz, 1.3H, major rotamer), 4.70 (t, J = 5.0 Hz, 0.7H, minor rotamer), 4.09 (t, J = 5.4 Hz, 1.3H, major rotamer), 3.97 (t, J = 5.0 Hz, 0.7H, minor |
| | | | rotamer). |
| 44 | 4-[6,7-dichloro- 10-(1H-pyrazol-4- yl)-3,4-dihydro- 1H-pyrazino[1,2- a]indol-2-yl]- N,N-dimethyl-4- oxo-butanamide | 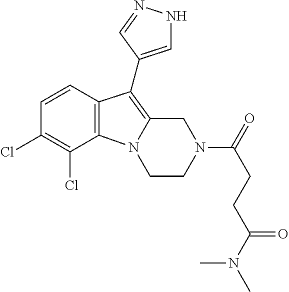 [SMILES: CN(C)C(=O)CCC(=O)N1CCn2c(c(-c3cn[nH]c3)c3ccc(Cl)c(Cl)c32)C1] | ES-MS (m/z): 434.0/436.0 (M + H) ¹H NMR (400 MHz, DMSO): 7.95 (br s, 0.6H, minor rotamer), 7.87 (br s, 1.4H, major rotamer), 7.67-7.60 (m, 1H), 7.27 (d, J = 8.5 Hz, 1H), 5.01 (br s, 0.6H, minor rotamer), 4.89 (br s, 1.4H, major rotamer), 4.75 (t, J = 5.2 Hz, 1.4H, major rotamer), 4.67-4.61 (m, 0.6H, minor rotamer), 4.05 (t, J = 5.2 Hz, 1.4H, major rotamer), 3.98-3.91 (m, 0.6H, minor rotamer), 2.97 (s, 3H), 2.78 (s, 3H), 2.70-2.60 (m, 2H), 2.57- 2.46 (m, 2H). |
| 45 | 1-[6,7-dichloro- 10-(1H-pyrazol-4- yl)-3,4-dihydro- 1H-pyrazino [1,2- a]indole-2-yl]-2- morpholino- ethanone | 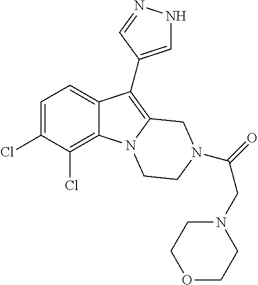 [SMILES: O=C(CN1CCOCC1)N1CCn2c(c(-c3cn[nH]c3)c3ccc(Cl)c(Cl)c32)C1] | ES-MS (m/z): 434.0/436.0 (M + H) ¹H NMR (400 MHz, DMSO): 13.09 (br s, 1H), 8.05 (br s, 0.3H, minor rotamer), 8.01 (br s, 0.7H, major rotamer), 7.80 (br s, 0.3H, minor rotamer), 7.74 (br s, 0.7H, major rotamer), 7.69 (d, J = 8.5 Hz, 0.3H, minor rotamer), 7.63 (d, J = 8.5 Hz, 0.7H, major rotamer), 7.27 (d, J = 8.5 Hz, 1H), 5.21 (s, 0.7H, minor rotamer), 4.89 (s, 1.3H, major rotamer), 4.76 (t, J = 5.3 Hz, 1.3H, major rotamer), 4.64 (t, J = 5.3 Hz, 0.7H, minor rotamer), 4.09 (t, J = 5.3 Hz, 1.3H, major rotamer), 3.95 (t, J = 5.3 Hz, 0.7H, minor rotamer), 3.63-3.51 (m, 2.6H, major rotamer), 3.34-3.26 (m, 1.4H, minor rotamer), |
| | | | 3.29 (s, 1.3H, major rotamer), 3.27 |
| | | | (s, 0.7H, minor rotamer), 2.46-2.38 |
| | | | (m, 2.6H, major rotamer), 2.32-2.26 |
| | | | (m, 1.4H, minor rotamer). |
| 46 | 1-[6,7-dichloro- 10-(1H-pyrazol-4- yl)-3,4-dihydro- 1H-pyrazino[1,2- a]indol-2-yl]-2- [(2-methyl-1,2,4- triazol-3- yl)methoxy]ethan one | 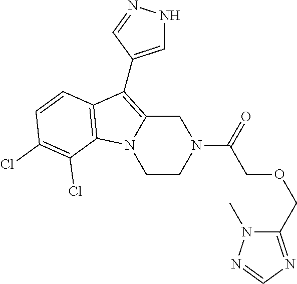 [SMILES: Cn1ncnc1COCC(=O)N1CCn2c(c(-c3cn[nH]c3)c3ccc(Cl)c(Cl)c32)C1] | ES-MS (m/z): 460.0/462.0 (M + H) ¹H NMR (400 MHz, DMSO): 7.95- 7.82 (m, 3H), 7.64 (d, J = 8.4 Hz, 1H), 7.28 (d, J = 8.5 Hz, 1H), 4.91 (s, 2H), 4.77-4.64 (m, 2H), 4.70 (s, 2H), 4.45 (s, 2H), 4.00-3.83 (m, 2H), 3.91 (s, 3H). |
| 47 | 2-[[6,7-dichloro- 10-(1H-pyrazol-4- yl)-3,4-dihydro- 1H-pyrazino[1,2- a]indol-2- yl]methyl]-5- (methoxymethyl)- 1,3,4-oxadiazole formic acid salt | 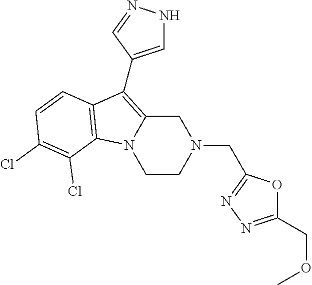 [SMILES: COCc1nnc(CN2CCn3c(c(-c4cn[nH]c4)c4ccc(Cl)c(Cl)c43)C2)o1] | ES-MS (m/z): 433.0/435.0 (M + H) ¹H NMR (400 MHz, DMSO): 13.05 (br s, 1H), 8.07-7.55 (m, 2H), 7.60 (d, J = 8.5 Hz, 1H), 7.25 (d, J = 8.5 Hz, 1H), 4.71-4.55 (m, 2H), 4.64 (s, 2H), 4.14 (s, 2H), 4.00 (s, 2H), 3.31 (s, 3H), 3.10 (t, J = 5.4 Hz, 2H). |
| 48 | 3-[2-[6,7- dichloro-10-(1H- pyrazol-4-yl)-3, 4-dihydro-1H- pyrazino[1,2- a]indol-2-oxo- ethyl]oxazolidin- 2-one | 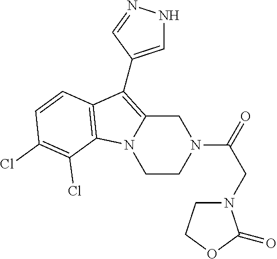 [SMILES: O=C(CN1CCOC1=O)N1CCn2c(c(-c3cn[nH]c3)c3ccc(Cl)c(Cl)c32)C1] | ES-MS (m/z): 434.0/436.0 (M + H) ¹H NMR (400 MHz, DMSO): 13.10 (br s, 1H), 8.18-7.60 (m, 2H), 7.67 (d, J = 8.5 Hz, 0.3H, minor rotamer), 7.64 (d, J = 8.5 Hz, 0.7H, major rotamer), 7.28 (d, J = 8.5 Hz, 1H), 4.98 (s, 0.7H, minor rotamer), 4.90 (s, 1.3H, major rotamer), 4.77 (t, J = 5.3 Hz, 1.3H, major rotamer), 4.72- 4.65 (m, 0.7H, minor rotamer), 4.33-4.23 (m, 4H), 4.03-3.93 (m, 2H), 3.55 (t, J = 8.0 Hz, 2H). |
| 49 | 1-[6,7-dichloro- 10-(1H-pyrazol-4- yl)-3,4-dihydro- 1H-pyrazino[1,2- a]indol-2-yl]-3- (1,2,4-triazol-1- yl)propan-1-one | 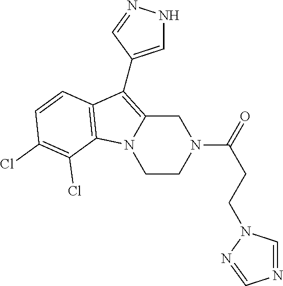 [SMILES: O=C(CCn1cncn1)N1CCn2c(c(-c3cn[nH]c3)c3ccc(Cl)c(Cl)c32)C1] | ES-MS (m/z): 434.0/436.0 (M + H) ¹H NMR (400 MHz, DMSO): 8.48 (s, 1H), 7.94 (br s, 0.7H, minor rotamer), 7.93 (s, 0.3H, minor rotamer), 7.88 (s, 0.7H, major rotamer), 7.87 (br s, 1.3H, major rotamer), 7.65 (d, J = 8.5 Hz, 0.3H, minor rotamer), 7.63 (d, J = 8.5 Hz, 0.7H, major rotamer), 7.27 (d, J = 8.5 Hz, 1H), 4.94 (s, 0.7H, minor rotamer), 4.89 (s, 1.3H, major rotamer), 4.71 (t, J = 5.4 Hz, 1.3H, major rotamer), 4.64 (t, J = 5.4 Hz, 0.7H, minor rotamer), 4.42 (t, J = 6.6 Hz, 2H), 3.98 (t, J = 5.4 Hz, 1.3H, major rotamer), 3.94 (t, J = 5.4 Hz, 0.7H, minor rotamer), 3.13-3.05 (m, 2H). |
| 50 | [6,7-dichloro-10- (1H-pyrazol-4- yl)-3,4-dihydro- 1H-pyrazino[1,2- a]indol-2-yl]-(4- methylmorpholin- 2-yl)methanone | 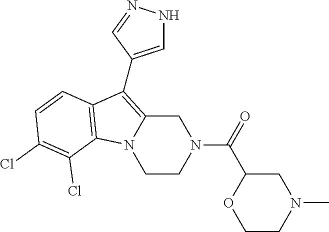 [SMILES: CN1CCOC(C(=O)N2CCn3c(c(-c4cn[nH]c4)c4ccc(Cl)c(Cl)c43)C2)C1] | ES-MS (m/z): 434.0/436.0 (M + H) ¹H NMR (400 MHz, DMSO): 12.94 (br s, 1H), 8.13 (s, 0.4H, formate), 7.88 (br s, 2H), 7.63 (d, J = 8.5 Hz, 1H), 7.28 (d, J = 8.5 Hz, 1H), 5.18- 4.83 (m, 2H), 4.82-4.58 (m, 2H), 4.51-4.35 (m, 1H), 4.14-3.67 (m, 3H), 3.63-3.43 (m, 1H), 2.96-2.62 (m, 2H), 2.46-2.00 (m, 5H). |
| 51 | [6,7-dichloro-10- (1H-pyrazol-4- yl)-3,4-dihydro- 1H-pyrazino[1,2- a]indol-2-yl]-(1,4- dioxan-2- yl)methanone | 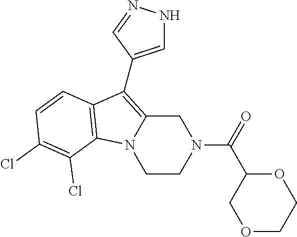 [SMILES: O=C(C1COCCO1)N1CCn2c(c(-c3cn[nH]c3)c3ccc(Cl)c(Cl)c32)C1] | ES-MS (m/z): 421.0/423.0 (M + H) ¹H NMR (400 MHz, DMSO): 7.88 (br s, 2H), 7.66-7.60 (m, 1H), 7.28 (d, J = 8.4 Hz, 1H), 5.19-4.56 (m, 4H), 4.54-4.45 (m, 1H), 4.16-3.87 (m, 2H), 3.82-3.74 (m, 2H), 3.71- 3.55 (m, 2H), 3.55-3.37 (m, 2H). |
| 52 | N-[2-[6,7- dichloro-10-(1H- pyrazol-4-yl)-3, 4-dihydro-1H- pyrazino[1,2- a]indol-2-yl]-2- oxo-ethyl]-N- methyl-acetamide | 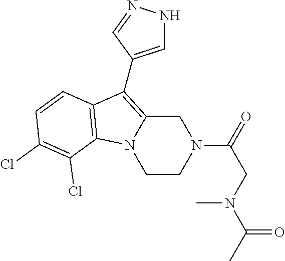 [SMILES: CC(=O)N(C)CC(=O)N1CCn2c(c(-c3cn[nH]c3)c3ccc(Cl)c(Cl)c32)C1] | ES-MS (m/z): 420.0/422.0 (M + H) ¹H NMR (400 MHz, DMSO): 7.96 (br s, 0.7H, rotamers 3 and 4), 7.88 (br s, 1.3H, rotamers 1 and 2), 7.70- 7.60 (m, 1H), 7.28 (d, J = 8.5 Hz, 0.45H, rotamers 2 and 4), 7.28 (d, J = 8.5 Hz, 0.55H, rotamers 1 and 3), 4.99 (s, 0.3H, rotamer 4), 4.96 (s, 0.4H, rotamer 3), 4.93 (s, 0.6H, rotamer 2), 4.89 (s, 0.7H, rotamer 1), 4.79 (t, J = 5.3 Hz, 0.7H, rotamer 2), 4.75 (t, J = 5.3 Hz, 0.6H, rotamer 1), 4.72-4.64 (m, 0.7H, rotamers 3 and 4), 4.48 (s, 0.3H, rotamer 4), 4.45 (s, 0.6H, rotamer 2), 4.30 (s, 1.1H, rotamers 1 and 3), 4.03-3.91 |
| | | | (m, 2H), 2.96 (s, 1.7H, rotamers 1 |
| | | | and 3), 2.76 (s, 0.5H, rotamer 4), |
| | | | 2.75 (s, 0.8H, rotamer 2), 2.01 (s, |
| | | | 1.7H, rotamers 1 and 3), 1.85 (s, |
| | | | 1.3H, rotamers 2 and 4). |
| 53 | 1-[6,7-dichloro- 10-(1H-pyrazol-4- yl)-3,4-dihydro- 1H-pyrazino[1,2- a]indol-2-yl]-2- hydroxy-ethanone | 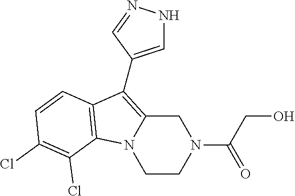 [SMILES: O=C(CO)N1CCn2c(c(-c3cn[nH]c3)c3ccc(Cl)c(Cl)c32)C1] | ES-MS (m/z): 365.0/367.0 (M + H) ¹H NMR (400 MHz, DMSO): 13.11 (br s, 1H), 8.10-7.94 (m, 1H), 7.85- 7.70 (m, 1H), 7.63 (d, J = 8.4 Hz, 1H), 7.27 (d, J = 8.5 Hz, 1H), 4.97- 4.85 (m, 3H), 4.77-4.63 (m, 2H), 4.23 (d, J = 5.4 Hz, 2H), 4.00-3.89 (m, 2H). |

### SCHEME FOR EXAMPLE 54

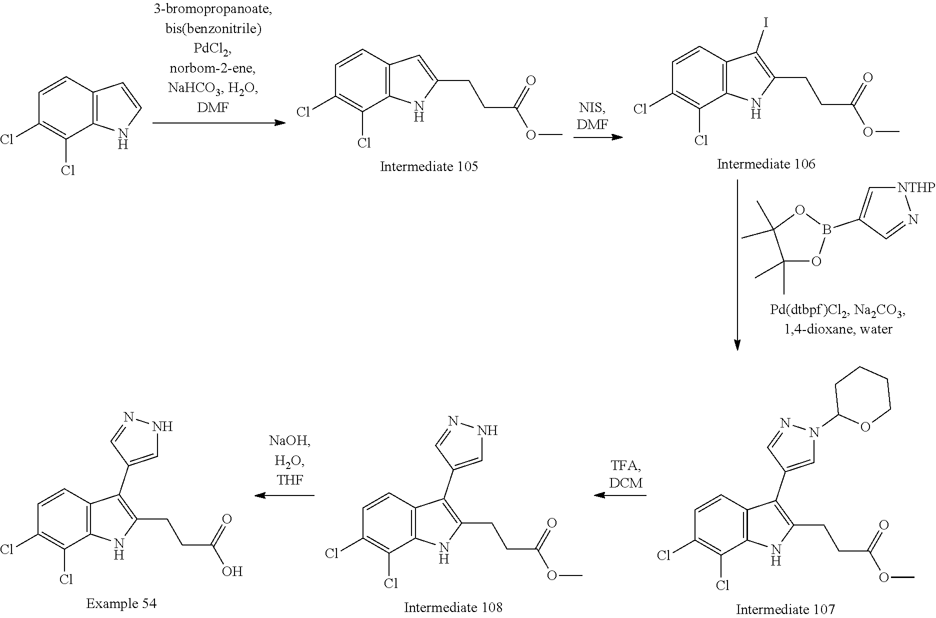

### Intermediate 105

### Methyl 3-(6,7-dichloro-1H-indol-2-yl)propanoate

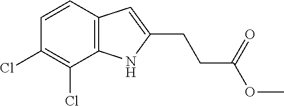 [SMILES: COC(=O)CCc1cc2ccc(Cl)c(Cl)c2[nH]1]

Suspend 6,7-dichloro-1H-indole (5.00 g, 25.5 mmol), methyl 3-bromopropanoate (5.22 g, 30.6 mmol), bis(benzonitrile)palladium chloride (1.55 g, 3.84 mmol), norborn-2-ene (9.71 g, 102 mmol) and sodium bicarbonate (8.60 g, 100 mmol) in water (690 mg, 38.3 mmol) and DMF (80 mL). Sparge with nitrogen and heat to 70° C. overnight. Cool to RT, concentrate, dilute with water and extract with EtOAc. Wash with water, brine, dry over anhydrous sodium sulfate, filter, and concentrate. Purify by flash column chromatography (EtOAc/petroleum ether) to yield methyl 3-(6, 7-dichloro-1H-indol-2-yl) propanoate (3.45 g, 12.0 mmol, 47% Yield).

### Intermediate 106

### Methyl 3-(6,7-dichloro-3-iodo-1H-indol-2-yl)propanoate

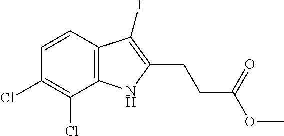 [SMILES: COC(=O)CCc1[nH]c2c(Cl)c(Cl)ccc2c1I]

Dissolve methyl 3-(6, 7-dichloro-1H-indol-2-yl) propanoate (230 mg, 0.715 mmol) in DMF (4 mL) and add NIS (181 mg, 0.788 mmol). Stir for 2 hours at RT. Dilute with EtOAc, wash with water and brine, dry over sodium sulfate, filter, and concentrate. Purify by flash column chromatography (EtOAc/petroleum ether) to yield methyl 3-(6, 7-dichloro-3-iodo-1H-indol-2-yl) propanoate (265 mg, 0.599 mmol, 83% Yield).

### Intermediate 107

### Methyl 3-(6,7-dichloro-3-(1-(tetrahydro-2H-pyran-2-yl)-1H-pyrazol-4-yl)-1H-indol-2-yl)propanoate

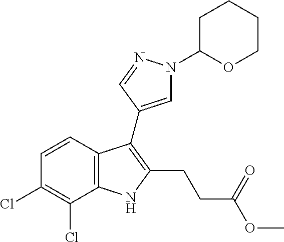 [SMILES: COC(=O)CCc1[nH]c2c(Cl)c(Cl)ccc2c1-c1cnn(C2CCCCO2)c1]

Suspend methyl 3-(6,7-dichloro-3-iodo-1H-indol-2-yl) propanoate (235 mg, 0.531 mmol), 1-(tetrahydro-2h-pyran-2-yl)-4-(4,4,5,5-tetramethyl-1,3,2-dioxaborolan-2-yl)-1h-pyrazole (226 mg, 0.796 mmol), Pd(dtbpf)Cl₂ (35 mg, 0.053 mmol) and sodium carbonate (113 mg, 1.06 mmol) in 1,4-dioxane (5 mL) and water (1 mL). Sparge with nitrogen and heat to 90° C. for 2 hours. Cool to RT, concentrate, dilute with water and extract with EtOAc. Dry over sodium sulfate, filter, and concentrate. Purify by flash column chromatography (EtOAc/petroleum ether) to yield methyl 3-[6, 7-dichloro-3-(1-tetrahydropyran-2-ylpyrazol-4-yl)-1H-indol-2-yl] propanoate (210 mg, 0.455 mmol, 86% Yield). ES-MS (m/z): 422.2/424.1 (M+H).

### Intermediate 108

### Methyl 3-(6,7-dichloro-3-(1H-pyrazol-4-yl)-1H-indol-2-yl)propanoate

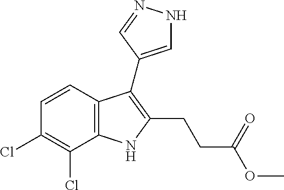 [SMILES: COC(=O)CCc1[nH]c2c(Cl)c(Cl)ccc2c1-c1cn[nH]c1]

Suspend methyl 3-[6, 7-dichloro-3-(1-tetrahydropyran-2-ylpyrazol-4-yl)-1H-indol-2-yl] propanoate (190 mg, 0.412 mmol) in DCM (2 mL) and add TFA (2 mL). Stir at RT for 2 hours and concentrate. Dilute with EtOAc, wash with saturated aqueous NaHCO₃ and water, dry over sodium sulfate, filter, and concentrate. Purify by flash silica column chromatography (EtOAc/petroleum ether) to yield methyl 3-[6, 7-dichloro-3-(1H-pyrazol-4-yl)-1H-indol-2-yl] propanoate (160 mg, 0.449 mmol, 98% Yield).

### EXAMPLE 54

### 3-(6,7-Dichloro-3-(1H-pyrazol-4-yl)-1H-indol-2-yl) propanoic acid

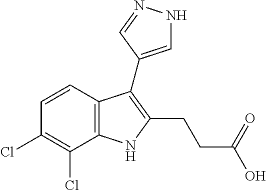 [SMILES: O=C(O)CCc1[nH]c2c(Cl)c(Cl)ccc2c1-c1cn[nH]c1]

Suspend methyl 3-[6,7-dichloro-3-(1H-pyrazol-4-yl)-1H-indol-2-yl] propanoate (140 mg, 0.393 mmol) in water (2 mL) and THF (2 mL). Add sodium hydroxide (157 mg, 3.92 mmol). Stir at RT for 2 hours. Acidify with 1N aqueous HCl to pH 4 and concentrate. Purify by prep-HPLC to yield methyl 3-(6, 7-dichloro-1H-indol-2-yl) propanoate (440 mg, 1.62 mmol, 14% Yield). ES-MS (m/z): 324.0/326.0 (M+H). ¹H NMR (400 MHz, DMSO): 12.67 (br s, 1H), 11.50 (s, 1H), 7.83 (s, 2H), 7.48 (d, J=8.5 Hz, 1H), 7.17 (d, J=8.5 Hz, 1H), 3.05 (dd, J=8.4, 7.5 Hz, 2H), 2.66 (dd, J=8.4, 7.5 Hz, 2H).

### SCHEME FOR EXAMPLE 55

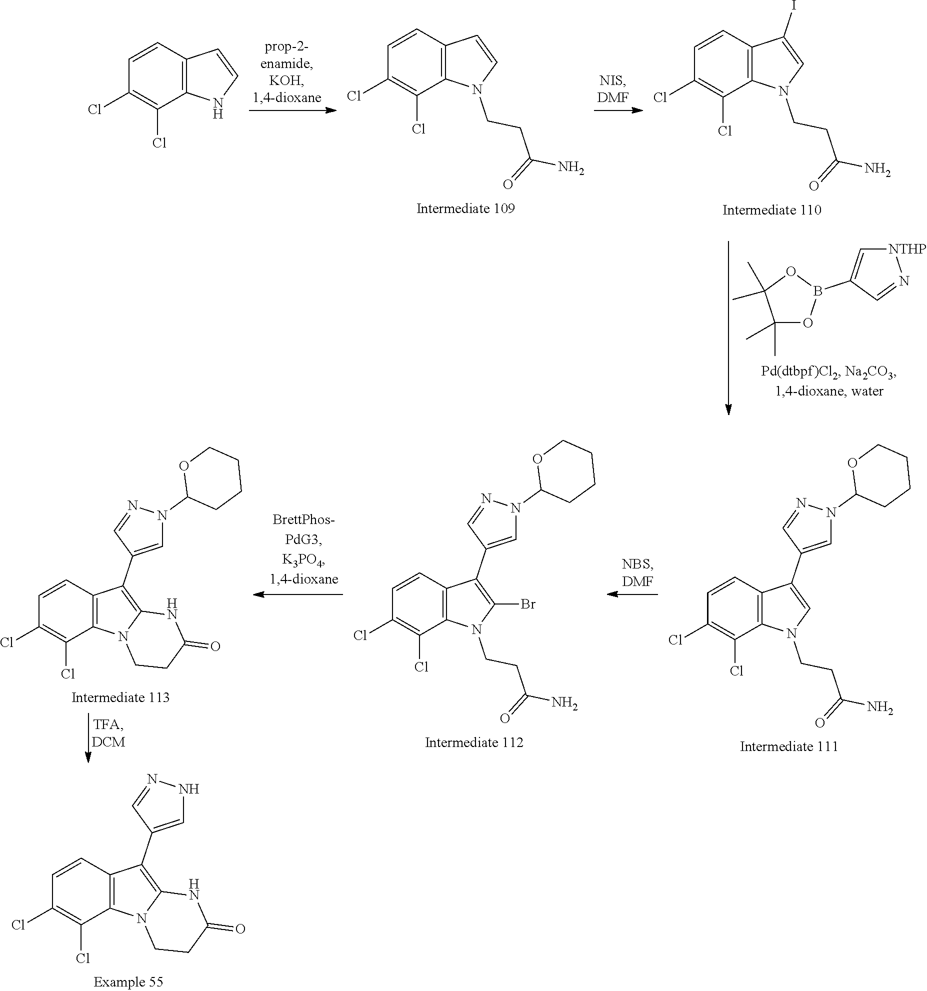

### Intermediate 109

### 3-(6, 7-Dichloroindol-1-yl)propanamide

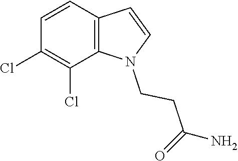 [SMILES: NC(=O)CCn1ccc2ccc(Cl)c(Cl)c21]

Dissolve 6, 7-dichloro-1H-indole (3.00 g, 14.5 mmol) in 1, 4-dioxane (120 mL). Add prop-2-enamide (1.50 g, 21.0 mmol) and KOH (960 mg, 14.5 mmol) at 0° C. Stir 12 hours at RT. Add water, extract with DCM, dry over anhydrous sodium sulfate, filter, and concentrate. Purify by flash column chromatography (EtOAc/petroleum ether) to yield 3-(6, 7-dichloroindol-1-yl)propanamide (2.50 g, 9.20 mmol, 64% Yield).

### Intermediate 110

### 3-(6, 7-Dichloro-3-iodo-indol-1-yl)propanamide

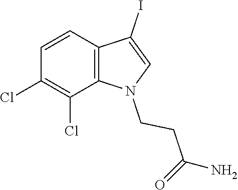 [SMILES: NC(=O)CCn1cc(I)c2ccc(Cl)c(Cl)c21]

Dissolve 3-(6, 7-dichloroindol-1-yl)propanamide (2.50 g, 9.20 mmol) in DMF (50 mL) and add NIS (2.30 g, 10.0 mmol). Stir for 2 hours at 80° C. Quench with water and stir vigorously for 15 minutes. Dilute with EtOAc, wash with brine, dry over anhydrous sodium sulfate, filter, and concentrate. Purify by flash column chromatography (EtOAc/petroleum ether) to yield 3-(6, 7-dichloro-3-iodo-indol-1-yl)propanamide (3.50 g, 8.20 mmol, 89% Yield).

### Intermediate 111

### 3-[6,7-Dichloro-3-(1-tetrahydropyran-2-ylpyrazol-4-yl)indol-1-yl]propanamide

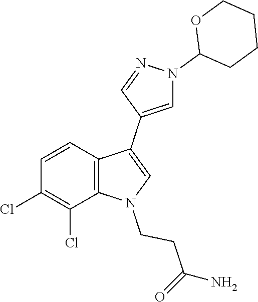 [SMILES: NC(=O)CCn1cc(-c2cnn(C3CCCCO3)c2)c2ccc(Cl)c(Cl)c21]

Suspend 3-(6,7-dichloro-3-iodo-indol-1-yl)propanamide (3.50 g, 8.20 mmol), 1-(tetrahydro-2H-pyran-2-yl)-4-(4,4,5,5-tetramethyl-1,3,2-dioxaborolan-2-yl)-1H-pyrazole (3.50 g, 12.0 mmol), PdCl₂(dtbpf) (550 mg, 0.830 mmol) and sodium carbonate (1.75 g, 16.4 mmol) in 1,4-dioxane (80 mL) and water (8 mL). Sparge with nitrogen and heat to 90° C. for 12 hours. Cool to RT, dilute with EtOAc, wash sequentially with saturated aqueous sodium bicarbonate and brine, dry over sodium sulfate, filter, and concentrate. Purify by flash column chromatography (EtOAc/petroleum ether) to yield 3-[6, 7-dichloro-3-(1-tetrahydropyran-2-ylpyrazol-4-yl)indol-1-yl]propanamide (1.57 g, 3.82 mmol, 46% Yield).

### Intermediate 112

### 3-[2-Bromo-6, 7-dichloro-3-(1-tetrahydropyran-2-ylpyrazol-4-yl)indol-1-yl]propanamide

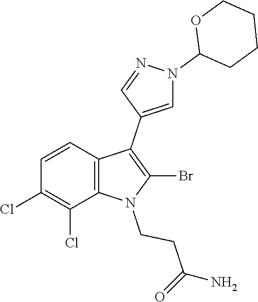 [SMILES: NC(=O)CCn1c(Br)c(-c2cnn(C3CCCCO3)c2)c2ccc(Cl)c(Cl)c21]

Dissolve 3-[6, 7-dichloro-3-(1-tetrahydropyran-2-ylpyrazol-4-yl)indol-1-yl]propanamide (1.57 g, 3.82 mmol) in DMF (30 mL) and add NBS (780 mg, 4.38 mmol). Stir at RT for 2 hours. Quench with water and stir vigorously for 15 min. Dilute with EtOAc, wash with brine, dry over anhydrous sodium sulfate, filter, and concentrate. Purify by flash column chromatography (EtOAc/petroleum ether) to yield 3-[2-bromo-6,7-dichloro-3-(1-tetrahydropyran-2-ylpyrazol-4-yl)indol-1-yl]propanamide (900 mg, 1.67 mmol, 44% Yield).

### Intermediate 113

### 6,7-Dichloro-10-(1-tetrahydropyran-2-ylpyrazol-4-yl)-3,4-dihydro-1H-pyrimido[1,2-a]indol-2-one

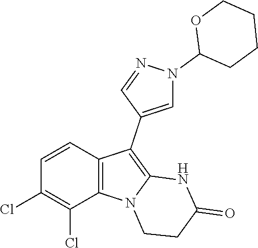 [SMILES: O=C1CCn2c(c(-c3cnn(C4CCCCO4)c3)c3ccc(Cl)c(Cl)c32)N1]

Suspend 3-[2-bromo-6,7-dichloro-3-(1-tetrahydropyran-2-ylpyrazol-4-yl)indol-1-yl]propanamide (900 mg, 1.76 mmol), potassium phosphate tribasic (2.30 g, 11.0 mmol) and BrettPhos-Pd-G₃ (490 mg, 0.53 mmol) in 1,4-dioxane (40 mL). Sparge with nitrogen and heat to 100° C. for 16 hours. Cool to RT, dilute with EtOAc, wash sequentially with saturated aqueous sodium bicarbonate and brine, dry over sodium sulfate, filter, and concentrate. Purify by prep-TLC (DCM/MeOH=10/1) to yield 6, 7-dichloro-10-(1-tetrahydropyran-2-ylpyrazol-4-yl)-3, 4-dihydro-1H-pyrimido[1,2-a]indol-2-one (90 mg, 0.080 mmol, 38% purity, 5% Yield). ES-MS (m/z): 405.0/407.0 (M+H).

### EXAMPLE 55

### 6,7-Dichloro-10-(1H-pyrazol-4-yl)-3,4-dihydro-1H-pyrimido[1,2-a]indol-2-one

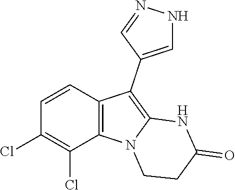 [SMILES: O=C1CCn2c(c(-c3cn[nH]c3)c3ccc(Cl)c(Cl)c32)N1]

Suspend 6, 7-dichloro-10-(1-tetrahydropyran-2-ylpyrazol-4-yl)-3, 4-dihydro-1H-pyrimido[1,2-a] indol-2-one (85 mg, 0.080 mmol, 38% purity) in DCM (2 mL). Add TFA (1 mL) and stir for 2 hours at RT. Concentrate and purify by prep-HPLC to yield 6, 7-dichloro-10-(1H-pyrazol-4-yl)-3,4-dihydro-1H-pyrimido[1,2-a]indol-2-one (4.01 mg, 0.011 mmol, 14% Yield) as a white solid. ES-MS (m/z): 321.2/323.2 (M+H). ¹H NMR (400 MHz, DMSO): 12.96 (br s, 1H), 10.49 (s, 1H), 7.98 (br s, 1H), 7.72 (br s, 1H), 7.44 (d, J=8.4 Hz, 1H), 7.22 (d, J=8.4 Hz, 1H), 4.81 (t, J=6.6 Hz, 2H), 2.83 (t, J=6.6 Hz, 2H).

### SCHEME FOR EXAMPLE 56

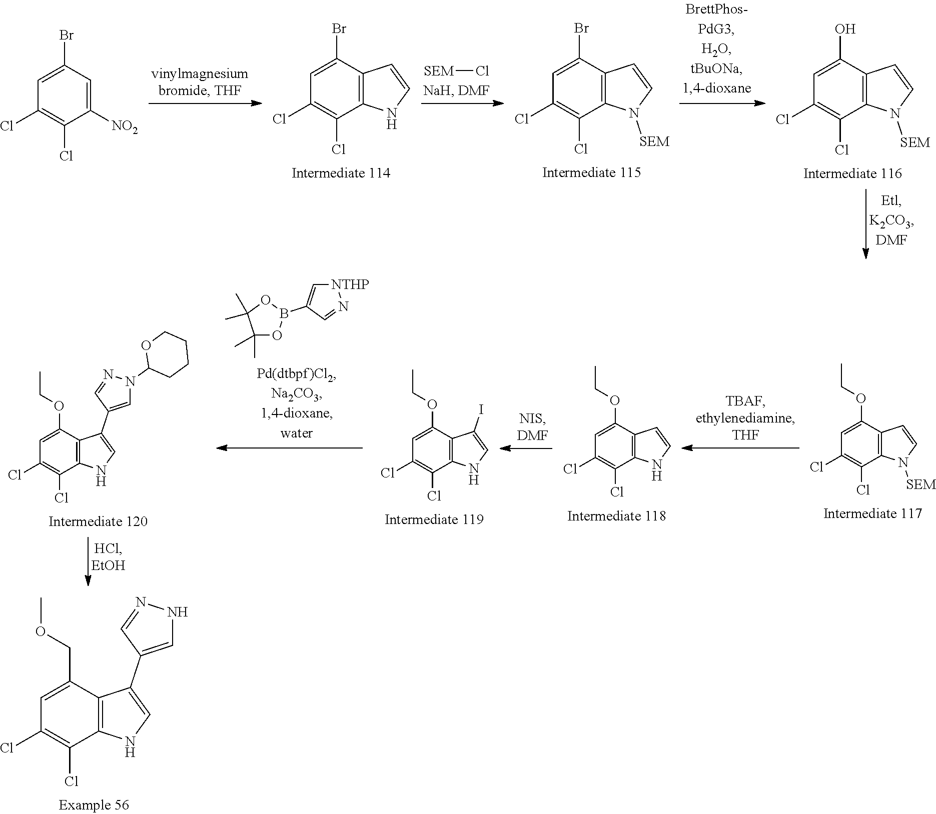

### Intermediate 114

### 4-Bromo-6,7-dichloro-1H-indole

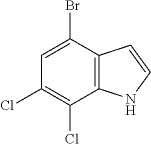 [SMILES: Clc1cc(Br)c2cc[nH]c2c1Cl]

Add vinylmagnesium bromide solution (1.0 mol/L) in THF (48.0 mL, 48.0 mmol) to 5-bromo-1,2-dichloro-3-nitro-benzene (4.33 g, 16.0 mmol, contains 50% undesired regioisomer 1-bromo-2,3-dichloro-4-nitro-benzene) in THF (47.0 g, 53 mL, 81.5, 53 mL) with stirring under nitrogen −40° C. Stir at −40° C. for 90 mins. Quench at −40° C. with saturated aqueous ammonium chloride. Extract with MTBE (2×). Wash the combined organics with water and brine, dry over anhydrous magnesium sulfate, filter, and concentrate. Purify by flash column chromatography (EtOAc/hexanes) to yield 4-bromo-6,7-dichloro-1H-indole (1.00 g, 3.70 mmol, 46%). ES-MS (m/z): 261.4/263.4/265.4 (M−H, negative ionization mode).

### Intermediate 115

### 2-[(4-Bromo-6,7-dichloro-indol-1-yl)methoxy]ethyl-trimethyl-silane

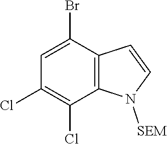 [SMILES: C[Si](C)(C)CCOCn1ccc2c(Br)cc(Cl)c(Cl)c21]

Dissolve 4-bromo-6,7-dichloro-1H-indole (5.00 g, 16.0 mmol, 85% purity) in DMF (100 mL) and add sodium hydride (1.00 g, 25.0 mmol, 60% in mineral oil) portion wise at 0° C. Stir for 30 min (until gas evolution stops) and add SEM-Cl (4.70 mL, 25.0 mmol). Stir at RT overnight. Quench with saturated aqueous ammonium chloride. Add EtOAc, wash sequentially with water and brine, dry over anhydrous sodium sulfate, filter, and concentrate. Purify by flash column chromatography (EtOAc/petroleum ether) to yield 2-[(4-bromo-6,7-dichloro-indol-1-yl)methoxy]ethyl-trimethyl-silane (2.40 g, 5.80 mmol, 36% Yield).

### Intermediate 116

### 6,7-Dichloro-1-(2-trimethylsilylethoxymethyl)indol-4-ol

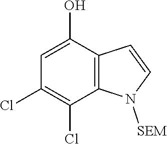 [SMILES: C[Si](C)(C)CCOCn1ccc2c(O)cc(Cl)c(Cl)c21]

Dissolve 2-[(4-bromo-6,7-dichloro-indol-1-yl)methoxy]ethyl-trimethyl-silane (1.40 g, 3.40 mmol) in 1,4-dioxane (20 mL) and add t-BuONa (830 mg, 8.38 mmol), H₂O (6 mL) and t-BuBrettPhos-Pd-G₃ (300 mg, 0.34 mmol) under N₂. Stir at 65° C. for 2.5 hours. Quench with saturated aqueous ammonium chloride. Add EtOAc, wash sequentially with water and brine, dry over anhydrous sodium sulfate, filter, and concentrate. Purify by flash column chromatography (EtOAc/petroleum ether) to yield 6,7-dichloro-1-(2-trimethylsilylethoxymethyl)indol-4-ol (1.02 g, 2.92 mmol, 87% Yield).

### Intermediate 117

### 2-[(6,7-Dichloro-4-ethoxy-indol-1-yl)methoxy]ethyl-trimethyl-silane

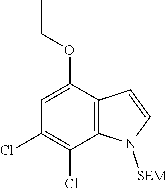 [SMILES: CCOc1cc(Cl)c(Cl)c2c1ccn2COCC[Si](C)(C)C]

Dissolve 6,7-dichloro-1-(2-trimethylsilylethoxymethyl)indol-4-ol (400 mg, 1.14 mmol) in DMF (8 mL). Add potassium carbonate (205 mg, 1.48 mmol) and iodoethane (0.18 mL, 2.20 mmol). Stir at RT for 1.5 hours under N₂. Quench with saturated lithium chloride. Add EtOAc, wash sequentially with water and brine, dry over anhydrous sodium sulfate, filter, and concentrate. Purify by flash column chromatography (EtOAc/petroleum ether) to yield 2-[(6,7-dichloro-4-ethoxy-indol-1-yl)methoxy]ethyl-trimethyl-silane (394 mg, 0.983 mmol, 86% Yield).

### Intermediate 118

### 6,7-Dichloro-4-ethoxy-1H-indole

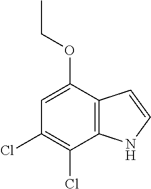

Dissolve 2-[(6,7-dichloro-4-ethoxy-indol-1-yl)methoxy]ethyl-trimethyl-silane (350 mg, 0.874 mmol) in THF (3.5 mL). Add ethylenediamine (352 μL, 5.25 mmol) and TBAF (9 mL, 9 mmol, 1.0 M in THF). Stir at 80° C. for 16 hours. Quench with saturated aqueous ammonium chloride. Add EtOAc, wash sequentially with water and brine, dry over anhydrous sodium sulfate, filter, and concentrate. Purify by flash column chromatography (EtOAc/petroleum ether) to yield 6,7-dichloro-4-ethoxy-1H-indole (102 mg, 0.400 mmol, 46% Yield).

### Intermediate 119

### 6,7-Dichloro-4-ethoxy-3-iodo-1H-indole

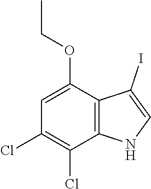

Dissolve 6,7-dichloro-4-ethoxy-1H-indole (102 mg, 0.446 mmol) in DMF (3 mL) and add NIS (133 mg, 0.579 mmol) at 0° C. Stir at RT for 1 hour. Add EtOAc, wash sequentially with water and brine, dry over anhydrous sodium sulfate, filter, and concentrate. Purify by flash column chromatography (EtOAc/petroleum ether) to yield 6,7-dichloro-4-ethoxy-3-iodo-1H-indole (76 mg, 0.21 mmol, 47% Yield).

### Intermediate 120

### 6,7-Dichloro-4-ethoxy-3-(1-tetrahydropyran-2-ylpyrazol-4-yl)-1H-indole

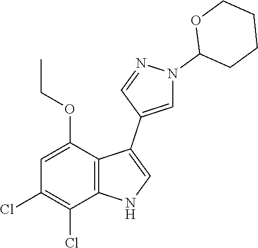 [SMILES: CCOc1cc(Cl)c(Cl)c2[nH]cc(-c3cnn(C4CCCCO4)c3)c12]

Dissolve 6,7-dichloro-4-ethoxy-3-iodo-1H-indole (A, 88 mg, 0.25 mmol) in 1,4-dioxane (1.5 mL) and add 1-(tetrahydro-2h-pyran-2-yL)-4-(4,4,5,5-tetramethyl-1,3,2-dioxaborolan-2-yl)-1H-pyrazole (70 mg, 0.25 mmol), sodium carbonate (79 mg, 0.74 mmol) in water (0.5 mL) and Pd(dtbpf)Cl₂ (33 mg, 0.050 mmol) at 25° C. under N₂. Sparge with nitrogen and heat to 90° C. for 0.5 hrs. Add EtOAc, wash sequentially with water and brine, dry over anhydrous sodium sulfate, filter, and concentrate. Purify by flash column chromatography (EtOAc/petroleum ether) to yield 6,7-dichloro-4-ethoxy-3-(1-tetrahydropyran-2-ylpyrazol-4-yl)-1H-indole (56 mg, 0.12 mmol, 48% Yield).

### EXAMPLE 56

### 6,7-Dichloro-4-ethoxy-3-(1H-pyrazol-4-yl)-1H-indole

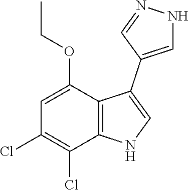 [SMILES: CCOc1cc(Cl)c(Cl)c2[nH]cc(-c3cn[nH]c3)c12]

Dissolve 6,7-dichloro-4-ethoxy-3-(1-tetrahydropyran-2-ylpyrazol-4-yl)-1H-indole (57 mg, 0.15 mmol) in 4 N HCl/EtOH (3 mL) and stir at RT for 1 hour. Concentrate and purify by prep-HPLC to yield 6,7-dichloro-4-ethoxy-3-(1H-pyrazol-4-yl)-1H-indole (11.25 mg, 0.037 mmol, 25% Yield). ES-MS (m/z): 295.9/297.9 (M+H). ¹H NMR (400 MHz, DMSO): 12.67 (br s, 1H), 11.63 (br s, 1H), 8.05-7.66 (m, 2H), 7.47 (d, J=2.5 Hz, 1H), 6.69 (s, 1H), 4.13 (q, J=6.9 Hz, 2H), 1.40 (t, J=6.9 Hz, 3H).

### SCHEME FOR EXAMPLE 57

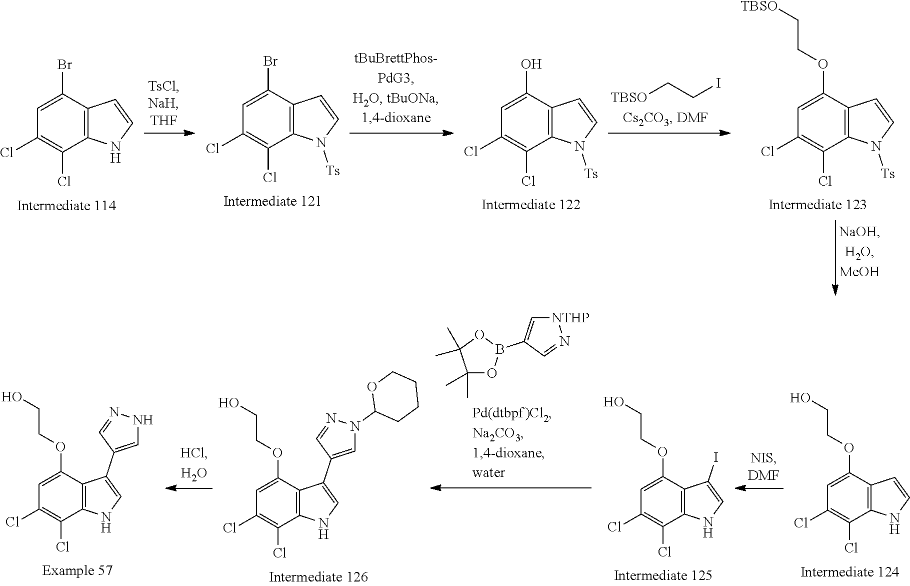

### Intermediate 121

### 4-Bromo-6,7-dichloro-1-(p-tolylsulfonyl)indole

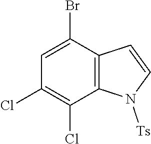 [SMILES: Cc1ccc(S(=O)(=O)n2ccc3c(Br)cc(Cl)c(Cl)c32)cc1]

Dissolve 4-bromo-6,7-dichloro-1H-indole (6.00 g, 20.4 mmol) in THF (50 mL). Add sodium hydride (1.7 g, 43 mmol, 60 mass % dispersion in oil) and stir 0.5 hour at RT. Add 4-methylbenzenesulfonyl chloride (5.83 g, 30.6 mmol) at 0° C. and allow to warm to RT overnight. Quench with water and extract with EtOAc (3×). Dry the combined organics over anhydrous sodium sulfate, filter, and concentrate. Purify by column chromatography (EtOAc/petroleum ether) to yield 4-bromo-6,7-dichloro-1-(p-tolylsulfonyl)indole (3.5 g, 7.9 mmol, 39% Yield).

### Intermediate 122

### 6,7-Dichloro-1-(p-tolylsulfonyl)indol-4-ol

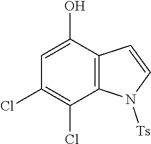 [SMILES: Cc1ccc(S(=O)(=O)n2ccc3c(O)cc(Cl)c(Cl)c32)cc1]

Dissolve 4-bromo-6,7-dichloro-1-(p-tolylsulfonyl)indole (3.5 g, 7.9 mmol) in 1,4-dioxane (25 mL). Add water (10 mL), [(2-di-tert-butylphosphino-3,6-dimethoxy-2′,4′,6′-triisopropyl-1,1′-biphenyl)-2-(2′-amino-1,1′-biphenyl)]palladium(II) methanesulfonate (0.71 g, 0.80 mmol) and sodium tert-butoxide (1.9 g, 20 mmol). Sparge with nitrogen and heat to 70° C. for 3 hours. Cool to RT, dilute with EtOAc and filter though diatomaceous earth. Dilute with water and extract with EtOAc (4×). Wash the combined organic extracts with brine, dry over anhydrous sodium sulfate, filter, and concentrate. Purify by flash column chromatography (EtOAc/petroleum ether) to yield 6,7-dichloro-1-(p-tolylsulfonyl)indol-4-ol (1.32 g, 3.34 mmol, 42% Yield).

### Intermediate 123

### 4-(2-((tert-Butyldimethylsilyl)oxy)ethoxy)-6,7-dichloro-1-tosyl-1H-indole

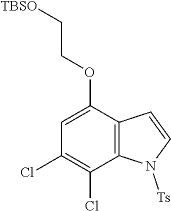 [SMILES: Cc1ccc(S(=O)(=O)n2ccc3c(OCCO[Si](C)(C)C(C)(C)C)cc(Cl)c(Cl)c32)cc1]

Dissolve 6,7-dichloro-1-tosyl-1H-indol-4-ol (500 mg, 1.26 mmol) in DMF (5 mL). Add cesium carbonate (620 mg, 1.90 mmol) and tert-butyl(2-iodoethoxy)dimethylsilane (760 mg, 2.52 mmol) at RT. Sparge with nitrogen and stir for 2 hours at 60° C. Add water, extract with EtOAc, dry over anhydrous sodium sulfate, filter and concentrate to yield 4-(2-((tert-butyldimethylsilyl)oxy)ethoxy)-6,7-dichloro-1-tosyl-1H-indole (926 mg, 1.26 mmol, 100% Yield).

### Intermediate 124

### 2-((6,7-Dichloro-1H-indol-4-yl)oxy)ethanol

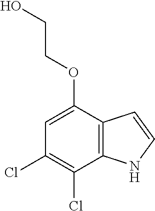 [SMILES: OCCOc1cc(Cl)c(Cl)c2[nH]ccc12]

Dissolve 4-(2-((tert-butyldimethylsilyl)oxy)ethoxy)-6, 7-dichloro-1-tosyl-1H-indole (920 mg, 1.25 mmol) in MeOH (12 mL) and water (4 mL). Add NaOH (506 mg, 12.5 mmol) at RT. Stir overnight at 60° C. Add water, extract with EtOAc, wash with 4% aqueous LiCl, dry over anhydrous sodium sulfate, filter, and concentrate. Purify by flash column chromatography (EtOAc/petroleum ether) to yield 2-((6,7-dichloro-1H-indol-4-yl)oxy)ethanol (240 mg, 0.926 mmol, 74% Yield).

### Intermediate 125

### 2-((6,7-Dichloro-3-iodo-1H-indol-4-yl)oxv)ethanol

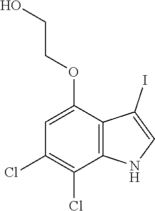 [SMILES: OCCOc1cc(Cl)c(Cl)c2[nH]cc(I)c12]

Dissolve 2-((6,7-dichloro-1H-indol-4-yl)oxy)ethanol (240 mg, 0.926 mmol) in DMF (5 mL) and add NIS (260 mg, 1.12 mmol) at 0° C. Stir for 1 hour at RT. Quench with saturated aqueous Na₂SO₃, extract with EtOAc, wash with 4% aqueous LiCl, dry over anhydrous sodium sulfate, filter, and concentrate to yield 2-((6,7-dichloro-3-iodo-1H-indol-4-yl)oxy)ethanol (330 mg, 0.843 mmol, 91% Yield).

### Intermediate 126

### 2-((6,7-Dichloro-3-(1-(tetrahydro-2H-pyran-2-yl)-1H-pyrazol-4-yl)-1H-indol-4-yl)oxy)ethanol

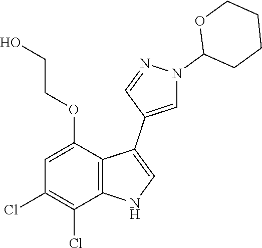 [SMILES: OCCOc1cc(Cl)c(Cl)c2[nH]cc(-c3cnn(C4CCCCO4)c3)c12]

Suspend 2-((6,7-dichloro-3-iodo-1H-indol-4-yl)oxy)ethanol (330 mg, 0.843 mmol), 1-(tetrahydro-2H-pyran-2-yl)-4-(4, 4, 5, 5-tetramethyl-1, 3, 2-dioxaborolan-2-yl)-1H pyrazole (380 mg, 1.34 mmol), Pd(dtbpf)Cl₂ (120 mg, 0.180 mmol) and Na₂CO₃ (290 mg, 2.74 mmol) in 1,4-Dioxane (6 mL) and water (2 mL). Sparge with nitrogen and heat to 90° C. for 30 min. Cool to RT, dilute with EtOAc, wash with brine, dry over sodium sulfate, filter, and concentrate. Purify by flash column chromatography (EtOAc/petroleum ether) to yield 2-((6,7-dichloro-3-(1-(tetrahydro-2H-pyran-2-yl)-1H-pyrazol-4-yl)-1H-indol-4-yl)oxy)ethanol (230 mg, 0.522 mmol, 62% Yield).

### EXAMPLE 57

### 2-((6,7-Dichloro-3-(1H-pyrazol-4-yl)-1H-indol-4-yl)oxy)ethanol

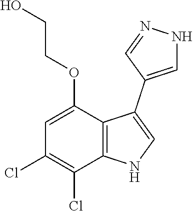 [SMILES: OCCOc1cc(Cl)c(Cl)c2[nH]cc(-c3cn[nH]c3)c12]

Suspend 2-((6, 7-dichloro-3-(1-(tetrahydro-2H-pyran-2-yl)-1H-pyrazol-4-yl-1H-indol-4-yl)oxy) ethanol (230 mg, 0.522 mmol) in 6 N aqueous HCl (4 mL) and stir for 1 hour at RT. Concentrate and purify by prep-HPLC to yield 2-((6,7-dichloro-3-(1H-pyrazol-4-yl)-1H-indol-4-yl)oxy)ethanol. (71 mg, 0.23 mmol, 43% Yield). ES-MS (m/z): 312.2/314.2 (M+H). ¹H NMR (400 MHz, DMSO): 12.63 (br s, 1H), 11.62 (s, 1H), 8.33-7.67 (m, 2H), 7.51 (s, 1H), 6.71 (s, 1H), 4.97 (t, J=5.2 Hz, 1H), 4.13 (t, J=4.7 Hz, 2H), 3.80 (q, J=4.6 Hz, 2H).

### SCHEME FOR EXAMPLE 58

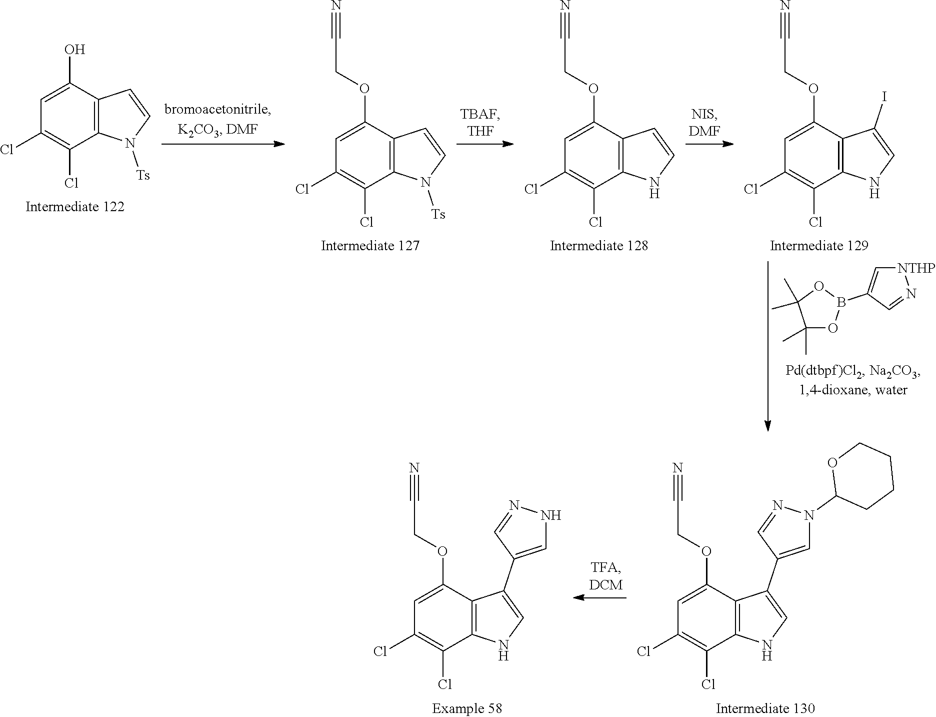

### Intermediate 127

### 2-[6, 7-Dichloro-1-(p-tolylsulfonyl)indol-4-yl]oxyacetonitrile

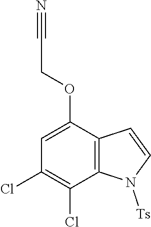 [SMILES: Cc1ccc(S(=O)(=O)n2ccc3c(OCC#N)cc(Cl)c(Cl)c32)cc1]

Dissolve 6, 7-dichloro-1-(p-tolylsulfonyl)indol-4-ol (500 mg, 1.26 mmol) in DMF (3 mL). Add bromoacetonitrile (313 mg, 2.53 mmol) and potassium carbonate (175 mg, 1.27 mmol) at 0° C. and stir for 2 hours. Add water, extract with EtOAc, dry over anhydrous sodium sulfate, filter, and concentrate. Purify by flash column chromatography (EtOAc/petroleum ether) to yield 2-[6, 7-dichloro-1-(p-tolylsulfonyl)indol-4-yl]oxyacetonitrile (1.05 g, 2.12 mmol, 80% purity).

### Intermediate 128

### 2-[(6, 7-Dichloro-1H-indol-4-yl)oxy]acetonitrile

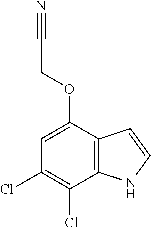 [SMILES: N#CCOc1cc(Cl)c(Cl)c2[nH]ccc12]

Dissolve 2-[6, 7-dichloro-1-(p-tolylsulfonyl)indol-4-yl]oxyacetonitrile (1.05 g, 2.12 mmol, 80% purity) in THF (5 mL) and add TBAF (10 mL, 10.0 mmol, 1M in THF). Stir at RT for 2 hours. Quench with saturated aqueous ammonium chloride, extract with EtOAc, dry over anhydrous sodium sulfate, filter, and concentrate to yield 2-[(6, 7-dichloro-1H-indol-4-yl)oxy]acetonitrile (801 mg, 2.69 mmol). ES-MS (m/z): 240.8/242.8 (M+H).

### Intermediate 129

### 2-[(6, 7-dichloro-3-iodo-1H-indol-4-yl)oxy]acetonitrile

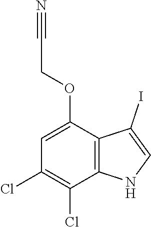 [SMILES: N#CCOc1cc(Cl)c(Cl)c2[nH]cc(I)c12]

Dissolve 2-[(6, 7-dichloro-1H-indol-4-yl)oxy]acetonitrile (801 mg, 2.69 mmol) in DMF (5 mL). Add NIS (742 mg, 3.23 mmol) and stir for 1.5 hours at RT. Add water, extract with EtOAc, dry over anhydrous sodium sulfate, filter, and concentrate. Purify by flash column chromatography (EtOAc/petroleum ether) to yield 2-[(6, 7-dichloro-3-iodo-1H-indol-4-yl)oxy]acetonitrile (923 mg, 1.89 mmol, 70% Yield).

### Intermediate 130

### 2-[[6, 7-Dichloro-3-(1-tetrahydropyran-2-ylpyrazol-4-yl)-1H-indol-4-yl]oxy]acetonitrile

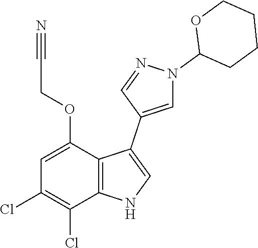 [SMILES: N#CCOc1cc(Cl)c(Cl)c2[nH]cc(-c3cnn(C4CCCCO4)c3)c12]

Suspend 2-[(6, 7-dichloro-3-iodo-1H-indol-4-yl)oxy]acetonitrile (923 mg, 2.01 mmol), 1-(tetrahydro-2H-pyran-2-yl)-4-(4, 4, 5, 5-tetramethyl-1, 3, 2-dioxaborolan-2-yl)1H-pyrazole (1.20 g, 4.10 mmol), Pd(dtbpf)Cl₂ (310 mg, 0.402 mmol) and sodium carbonate (640 mg, 6.04 mmol) in 1,4-dioxane (8 mL) and water (2 mL). Sparge with nitrogen and heat to 90° C. for 2 hours. Cool to RT and extract with EtOAc. Wash sequentially with saturated aqueous sodium bicarbonate and brine, dry over anhydrous sodium sulfate, filter, and concentrate. Purify by flash column chromatography (EtOAc/petroleum ether) to yield 2-[[6, 7-dichloro-3-(1-tetrahydropyran-2-ylpyrazol-4-yl)-1H-indol-4-yl]oxy]acetonitrile (297 mg, 0.235 mmol, 12% Yield). ES-MS (m/z): 391.1/393.0 (M+H).

### EXAMPLE 58

### 2-[[6, 7-Dichloro-3-(1H-pyrazol-4-yl)-1H-indol-4-yl]oxy]acetonitrile

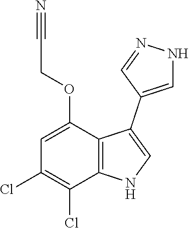 [SMILES: N#CCOc1cc(Cl)c(Cl)c2[nH]cc(-c3cn[nH]c3)c12]

Suspend 2-[[6, 7-dichloro-3-(1-tetrahydropyran-2-ylpyrazol-4-yl)-1H-indol-4-yl]oxy] acetonitrile (100 mg, 0.243 mmol) in DCM (3 mL). Add TFA (5 mL) and stir for 5 hours at RT. Concentrate and purify by prep-HPLC to yield 2-[[6, 7-dichloro-3-(1H-pyrazol-4-yl)-1H-indol-4-yl]oxy]acetonitrile (35 mg, 0.11 mmol, 46% Yield). ES-MS (m/z): 307.2/309.2 (M+H). ¹H NMR (400 MHz, DMSO): 12.75 (br s, 1H), 11.82 (br s, 1H), 7.97-7.95 (m, 2H), 7.55 (d, J=2.5 Hz, 1H), 6.94 (s, 1H), 5.31 (s, 2H).

### SCHEME FOR EXAMPLE 59

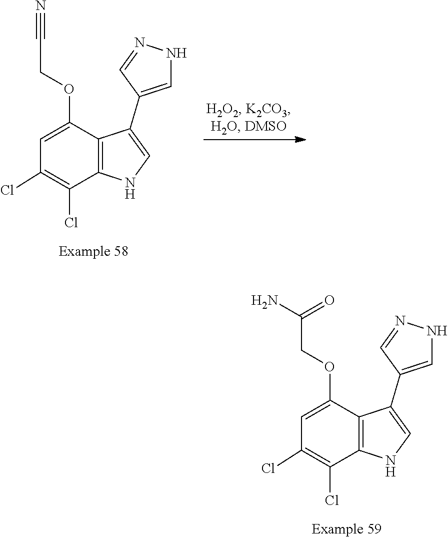 [SMILES: N#CCOc1cc(Cl)c(Cl)c2[nH]cc(-c3cn[nH]c3)c12 ; NC(=O)COc1cc(Cl)c(Cl)c2[nH]cc(-c3cn[nH]c3)c12]

### EXAMPLE 59

### 2-[[6,7-Dichloro-3-(1H-pyrazol-4-yl)-1H-indol-4-yl]oxy]acetamide

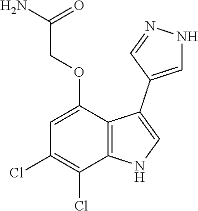 [SMILES: NC(=O)COc1cc(Cl)c(Cl)c2[nH]cc(-c3cn[nH]c3)c12]

Suspend 2-[[6,7-dichloro-3-(1H-pyrazol-4-yl)-1H-indol-4-yl]oxy]acetonitrile (170 mg, 0.443 mmol) and K₂CO₃ (160 mg, 1.16 mmol) in DMSO (3 mL). Add 30% aqueous H₂O₂ (200 mg, 1.76 mmol) at 0° C. and stir for 2 hours at RT. Extract with EtOAc, dry over anhydrous sodium sulfate, filter, and concentrate. Purify by HPLC to yield 2-[[6,7-dichloro-3-(1H-pyrazol-4-yl)-1H-indol-4-yl]oxy]acetamide (72 mg, 0.22 mmol, 50% Yield). ES-MS (m/z): 325.0/327.0 (M+H). ¹H NMR (400 MHz, DMSO): 12.69 (br s, 1H), 11.68 (d, J=1.9 Hz, 1H), 7.97 (br s, 2H), 7.49 (d, J=2.5 Hz, 1H), 7.40 (s, 1H), 7.27 (s, 1H), 6.63 (s, 1H), 4.59 (s, 2H).

### SCHEME FOR EXAMPLE 60

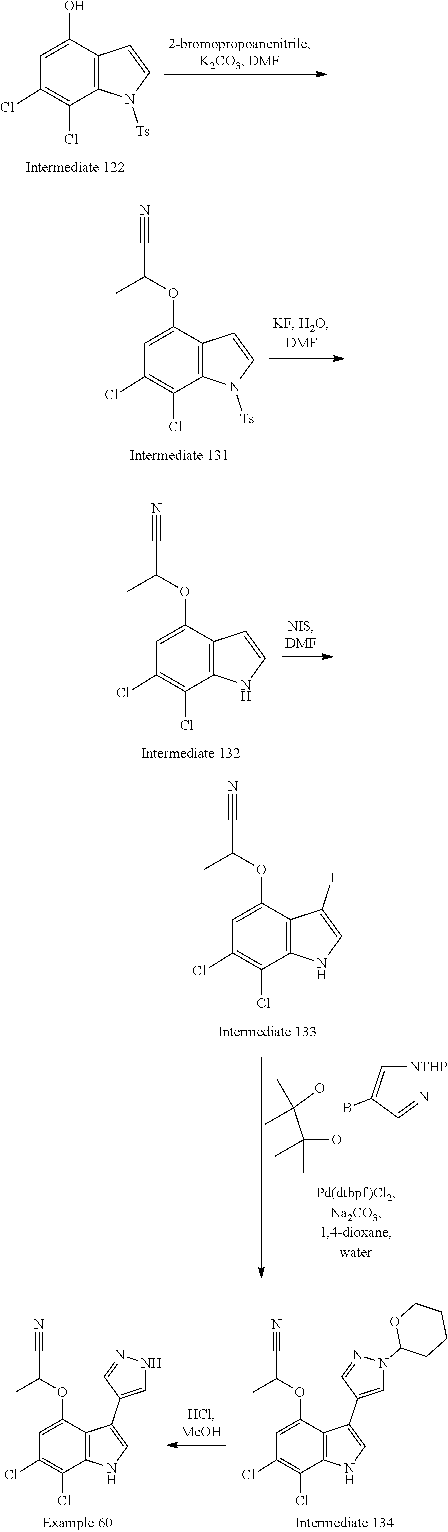

### Intermediate 131

### 2-[6, 7-Dichloro-1-(p-tolylsulfonyl)indol-4-yl]oxypropanenitrile

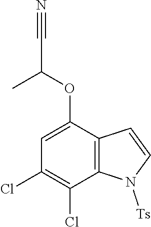 [SMILES: Cc1ccc(S(=O)(=O)n2ccc3c(OC(C)C#N)cc(Cl)c(Cl)c32)cc1]

Dissolve 6, 7-dichloro-1-(p-tolylsulfonyl)indol-4-ol (800 mg, 2.22 mmol) in DMF (15 mL). Add potassium carbonate (400 mg, 2.89 mmol) and 2-bromopropanenitrile (0.41 mL, 4.0 mmol). Stir for 1.5 hours at RT. Quench with water. Collect the precipitate by suction filtration, rinsing with acetonitrile, and dry under vacuum to yield 2-[6, 7-dichloro-1-(p-tolylsulfonyl)indol-4-yl]oxypropanenitrile (850 mg, 2.07 mmol, 93% Yield).

### Intermediate 132

### 2-[(6, 7-Dichloro-1H-indol-4-yl)oxy]propanenitrile

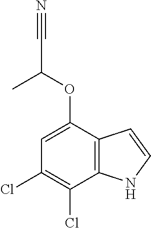 [SMILES: CC(C#N)Oc1cc(Cl)c(Cl)c2[nH]ccc12]

Dissolve 2-[6, 7-dichloro-1-(p-tolylsulfonyl)indol-4-yl]oxypropanenitrile (850 mg, 2.07 mmol) in DMF (8 mL) and add potassium fluoride (725 mg, 12.4 mmol) in water (8 mL). Stir overnight at 100° C. Quench with water and extract with EtOAc, wash with brine, dry over anhydrous sodium sulfate, filter, and concentrate. Purify by flash column chromatography (EtOAc/petroleum ether) to yield 2-[(6, 7-dichloro-1H-indol-4-yl)oxy]propanenitrile (500 mg, 1.47 mmol, 71% Yield).

### Intermediate 133

### 2-[(6, 7-Dichloro-3-iodo-1H-indol-4-yl)oxy]propanenitrile

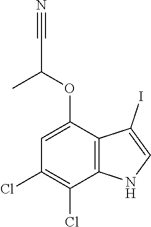 [SMILES: CC(C#N)Oc1cc(Cl)c(Cl)c2[nH]cc(I)c12]

Dissolve 2-[(6, 7-dichloro-1H-indol-4-yl)oxy]propanenitrile (500 mg, 1.47 mmol) in DMF (8 mL) and add NIS (410 mg, 1.76 mmol) at 0° C. Stir for 1 hour at RT. Quench with saturated aqueous Na₂S₂O₃ and extract with EtOAc. Wash with saturated aqueous sodium bicarbonate, dry over anhydrous sodium sulfate, filter, and concentrate. Purify by flash column chromatography (EtOAc/petroleum ether) to yield 2-[(6, 7-dichloro-3-iodo-1H-indol-4-yl)oxy]propanenitrile (480 mg, 0.944 mmol, 64% Yield).

### Intermediate 134

### 2-[[6,7-Dichloro-3-(1-tetrahydropyran-2-ylpyrazol-4-yl)-1H-indol-4-yl]oxy]propanenitrile

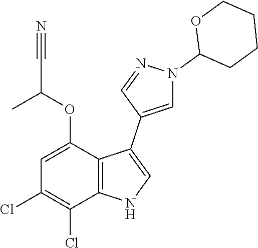 [SMILES: CC(C#N)Oc1cc(Cl)c(Cl)c2[nH]cc(-c3cnn(C4CCCCO4)c3)c12]

Suspend 2-[(6, 7-dichloro-3-iodo-1H-indol-4-yl)oxy]propanenitrile (480 mg, 0.944 mmol), 1-(tetrahydro-2h-pyran-2-yl)-4-(4,4,5,5-tetramethyl-1,3,2-dioxaborolan-2-yl)-1h-pyrazole (320 mg, 1.12 mmol), Pd(dtbpf)Cl₂ (125 mg, 0.188 mmol) and Na₂CO₃ (300 mg, 2.83 mmol) in 1,4-dioxane (9 mL) and water (3 mL). Sparge with nitrogen and heat to 90° C. for 20 min. Cool to RT, quench with water, dilute with EtOAc, wash with brine, dry over anhydrous sodium sulfate, filter, and concentrate. Purify by flash column chromatography (EtOAc/petroleum ether) to yield 2-[6, 7-dichloro-1-(p-tolylsulfonyl)indol-4-yl]oxypropanenitrile (150 mg, 0.183 mmol, 19% Yield) as a white solid. ES-MS (m/z): 405.0/407.0 (M+H).

### EXAMPLE 60

### 2-[[6, 7-Dichloro-3-(1H-pyrazol-4-yl)-1H-indol-4-yl]oxy]propanenitrile

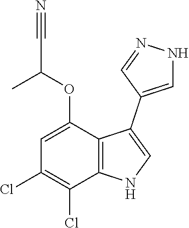 [SMILES: CC(C#N)Oc1cc(Cl)c(Cl)c2[nH]cc(-c3cn[nH]c3)c12]

Dissolve 2-[[6, 7-dichloro-3-(1-tetrahydropyran-2-ylpyrazol-4-yl)-1H-indol-4-yl]oxy] propane nitrile (100 mg, 0.234 mmol) in 4N HCl/MeOH (3 mL) and stir for 2 hours at RT. Concentrate and purify by prep-HPLC to yield 2-[[6,7-dichloro-3-(1H-pyrazol-4-yl)-1H-indol-4-yl]oxy]propanenitrile (5 mg, 0.016 mmol, 7% Yield). ES-MS (m/z): 321.2/323.2 (M+H). ¹H NMR (400 MHz, DMSO): 11.83 (br s, 1H), 7.80 (s, 2H), 7.51 (d, J=2.3 Hz, 1H), 7.00 (s, 1H), 5.59 (q, J=6.7 Hz, 1H), 1.70 (d, J=6.6 Hz, 3H).

### SCHEME FOR EXAMPLE 61

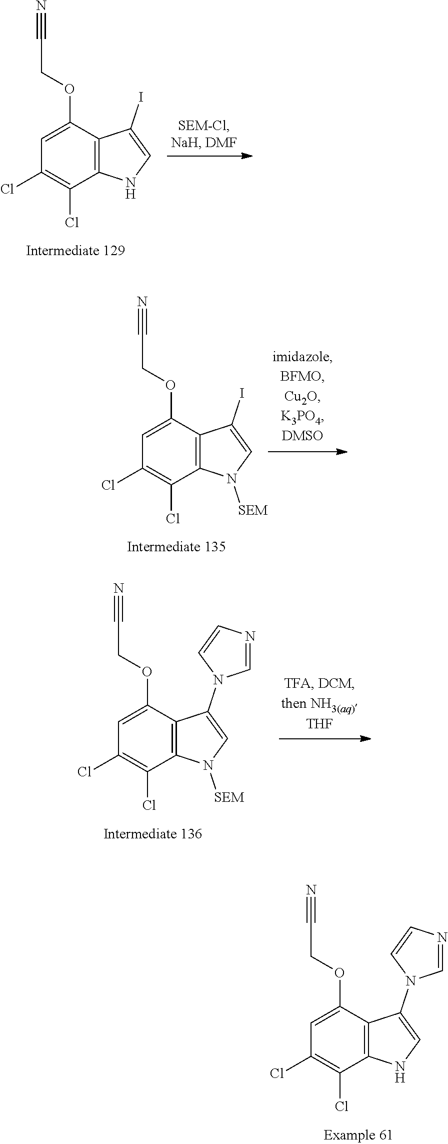 [SMILES: N#CCOc1cc(Cl)c(Cl)c2[nH]cc(I)c12 ; C[Si](C)(C)CCOCn1cc(I)c2c(OCC#N)cc(Cl)c(Cl)c21 ; C[Si](C)(C)CCOCn1cc(-n2ccnc2)c2c(OCC#N)cc(Cl)c(Cl)c21 ; N#CCOc1cc(Cl)c(Cl)c2[nH]cc(-n3ccnc3)c12]

### Intermediate 135

### 2-[6,7-Dichloro-3-iodo-1-(2-trimethylsilylethoxymethyl)indol-4-yl]oxyacetonitrile

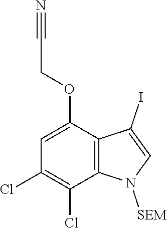 [SMILES: C[Si](C)(C)CCOCn1cc(I)c2c(OCC#N)cc(Cl)c(Cl)c21]

Suspend 2-[(6,7-dichloro-3-iodo-1H-indol-4-yl)oxy]acetonitrile (540 mg, 1.46 mmol) in DMF (8 mL). Add NaH (88 mg, 2.2 mmol, 60% dispersion in mineral oil) at 0° C. and stir for 1 hour. Add 2-(trimethylsilyl)ethoxymethyl chloride (372 mg, 2.19 mmol) at 0° C. and stir for 3 hours at RT. Quench with H₂O and extract with EtOAc. Wash with brine, dry over anhydrous sodium sulfate, filter, and concentrate. Purify by flash column chromatography (EtOAc/petroleum ether) to yield 2-[6, 7-dichloro-3-iodo-1-(2-trimethylsilylethoxymethyl)indol-4-yl]oxyacetonitrile (430 mg, 0.778 mmol, 53% Yield).

### Intermediate 136

### 2-[6,7-Dichloro-3-imidazol-1-yl-1-(2-trimethylsilylethoxymethyl)indol-4-yl]oxyacetonitrile

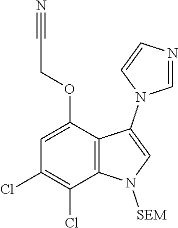 [SMILES: C[Si](C)(C)CCOCn1cc(-n2ccnc2)c2c(OCC#N)cc(Cl)c(Cl)c21]

Suspend 2-[6,7-dichloro-3-iodo-1-(2-trimethylsilylethoxymethyl)indol-4-yl]oxyacetonitrile (300 mg, 0.543 mmol), imidazole (75 mg, 1.1 mmol), Cu₂O (16 mg, 0.11 mmol), BFMO (56 mg, 0.22 mmol) and K₃PO₄ (233 mg, 1.09 mmol) in DMSO (6 mL). Sparge with nitrogen and stir at 120° C. for 1.5 hours under microwave. Cool to RT, dilute with EtOAc, wash sequentially with saturated aqueous sodium bicarbonate and brine, dry over sodium sulfate, filter, and concentrate. Purify by flash column chromatography (EtOAc/petroleum ether) to yield 2-[6,7-dichloro-3-imidazol-1-yl-1-(2-trimethylsilylethoxymethyl)indol-4-yl]oxyacetonitrile (50 mg, 0.096 mmol, 18% Yield). ES-MS (m/z): 437.0/439.1 (M+H).

### EXAMPLE 61

### 2-[(6,7-Dichloro-3-imidazol-1-yl-1H-indol-4-yl)oxy]acetonitrile

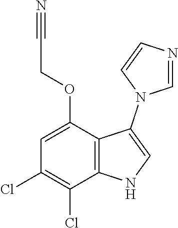 [SMILES: N#CCOc1cc(Cl)c(Cl)c2[nH]cc(-n3ccnc3)c12]

Suspend 2-[6, 7-dichloro-3-imidazol-1-yl-1-(2-trimethylsilylethoxymethyl)indol-4-yl]oxyaceto nitrile (40 mg, 0.077 mmol) in DCM (1.5 mL). Add TFA (0.75 mL) and stir for 1 hour at RT. Concentrate and dilute with THF (6 mL). Add 28% aqueous NH₃ (1 mL) and stir for 2 hours at RT. Concentrate and purify by prep-HPLC to yield 2-[(6,7-dichloro-3-imidazol-1-yl-1H-indol-4-yl)oxy]acetonitrile (13 mg, 0.044 mmol, 57% Yield). ES-MS (m/z): 307.0/309.0 (M+H). ¹H NMR (400 MHz, DMSO): 12.23 (s, 1H), 8.07 (br s, 1H), 7.78 (d, J=2.8 Hz, 1H), 7.49 (br s, 1H), 7.17 (br s, 1H), 7.05 (s, 1H), 5.18 (s, 2H).

### SCHEME FOR EXAMPLE 62

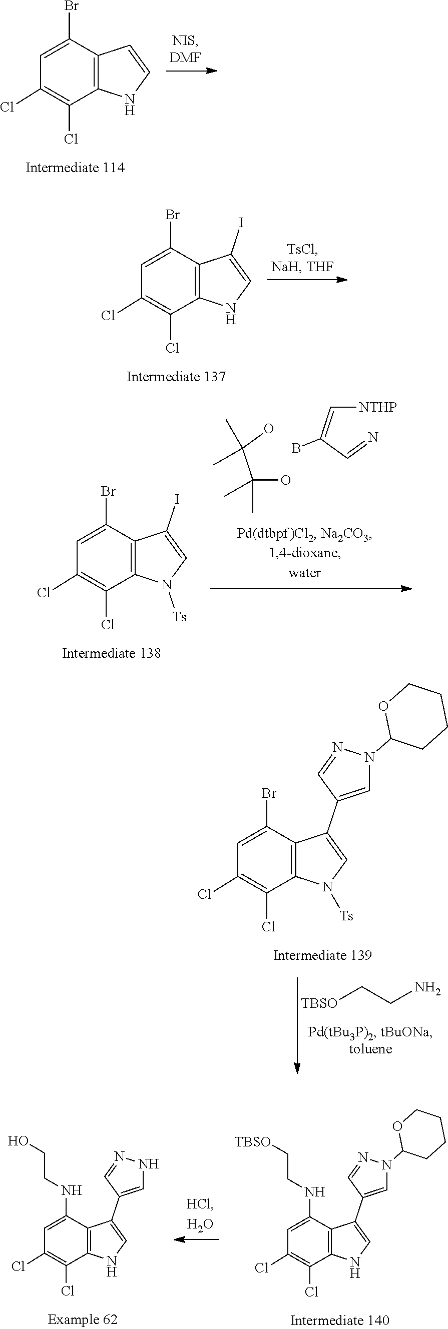

### Intermediate 137

### 4-Bromo-6, 7-dichloro-3-iodo-1H-indole

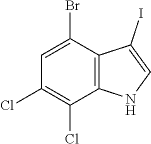 [SMILES: Clc1cc(Br)c2c(I)c[nH]c2c1Cl]

Dissolve 4-bromo-6, 7-dichloro-1H-indole (13.0 g, 36.8 mmol) in DMF (150 mL) and add NIS (9.30 g, 41.0 mmol). Stir for 4 hours at RT. Quench with water and stir vigorously for 15 minutes. Dilute with EtOAc, wash with brine, dry over sodium sulfate, filter, and concentrate. Purify by flash column chromatography (EtOAc/petroleum ether) to yield 4-bromo-6, 7-dichloro-3-iodo-1H-indole (13.2 g, 23.6 mmol, 64% Yield).

### Intermediate 138

### 4-Bromo-6, 7-dichloro-3-iodo-1-(p-tolylsulfonyl)indole

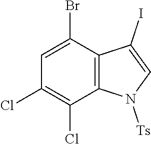 [SMILES: Cc1ccc(S(=O)(=O)n2cc(I)c3c(Br)cc(Cl)c(Cl)c32)cc1]

Dissolve 4-bromo-6, 7-dichloro-3-iodo-1H-indole (1.30 g, 23.3 mmol) in THF (200 mL) and add to a suspension of NaH (1.73 g, 43.3 mmol, 60% dispersion in mineral oil) in THF (100 mL) at 0° C. After stirring for 1 hour at 25° C., add TsCl (8.24 g, 43.2 mmol) at 0° C. Stir overnight at RT. Quench with water and stir vigorously for 15 min. Extract with EtOAc, wash with brine, dry over anhydrous sodium sulfate, filter, and concentrate. Triturate with EtOAc/petroleum ether (1:3) to yield 4-bromo-6, 7-dichloro-3-iodo-1-(p-tolylsulfonyl)indole (6.10 g, 10.0 mmol, 43% Yield).

### Intermediate 139

### 4-Bromo-6, 7-dichloro-1-(p-tolylsulfonyl)-3-(1-tetrahydropyran-2-ylpyrazol-4-yl)indole

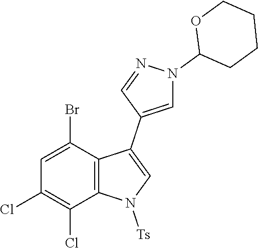 [SMILES: Cc1ccc(S(=O)(=O)n2cc(-c3cnn(C4CCCCO4)c3)c3c(Br)cc(Cl)c(Cl)c32)cc1]

Suspend 4-bromo-6, 7-dichloro-3-iodo-1-(p-tolylsulfonyl)indole (5.10 g, 8.42 mmol), 1-tetrahydropyran-2-yl-4-(4, 4, 5, 5-tetramethyl-1, 3, 2-dioxaborolan-2-yl)pyrazole (2.81 g, 10.1 mmol), Pd(dtbpf)Cl₂ (650 mg, 0.844 mmol), Na₂CO₃ (2.70 g, 25.0 mmol) in 1,4-dioxane (90. mL) and water (15 mL). Sparge with nitrogen and heat to 90° C. for 1 hour. Cool to RT, dilute with EtOAc, wash sequentially with saturated aqueous sodium bicarbonate and brine, dry over sodium sulfate, filter, and concentrate. Purify by flash column chromatography (EtOAc/petroleum ether) and triturate with CH₃CN to yield 4-bromo-6, 7-dichloro-1-(p-tolylsulfonyl)-3-(1-tetrahydropyran-2-ylpyrazol-4-yl)indole (3.00 g, 6.01 mmol, 59% Yield). ES-MS (m/z): 568.0/570.0/571.9 (M+H).

### Intermediate 140

### N-[2-[tert-Butyl(dimethyl)silyl]oxyethyl]-6, 7-dichloro-3-(1-tetrahydropyran-2-ylpyrazol-4-yl)-1H-indol-4-amine

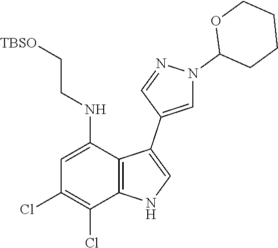 [SMILES: CC(C)(C)[Si](C)(C)OCCNc1cc(Cl)c(Cl)c2[nH]cc(-c3cnn(C4CCCCO4)c3)c12]

Suspend 4-bromo-6, 7-dichloro-1-(p-tolylsulfonyl)-3-(1-tetrahydropyran-2-ylpyrazol-4-yl)indole (500 mg, 0.834 mmol), 2-((tert-butyldimethylsilyl)oxy)ethanamine (465 mg, 2.52 mmol), (t-Bu₃P)₂Pd (130 mg, 0.249 mmol) and t-BuONa (290 mg, 2.93 mmol) in toluene (30 mL). Stir overnight at 100° C. under N₂. Cool to RT, dilute with EtOAc, wash sequentially with saturated aqueous sodium bicarbonate and brine, dry over sodium sulfate, filter, and concentrate. Purify by flash column chromatography (EtOAc/petroleum ether) to yield N-[2-[tert-butyl(dimethyl)silyl]oxyethyl]-6,7-dichloro-3-(1-tetrahydropyran-2-ylpyrazol-4-yl)-1H-indol-4-amine (160 mg, 0.273 mmol, 32% yield). ES-MS (m/z): 509.2/511.2 (M+H).

### EXAMPLE 62

### 2-[[6, 7-Dichloro-3-(1H-pyrazol-4-yl)-1H-indol-4-yl]amino]ethanol

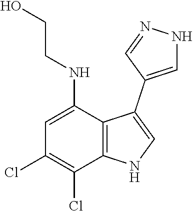 [SMILES: OCCNc1cc(Cl)c(Cl)c2[nH]cc(-c3cn[nH]c3)c12]

Dissolve N-[2-[tert-butyl(dimethyl)silyl]oxyethyl]-6,7-dichloro-3-(1-tetrahydropyran-2-ylpyrazol-4-yl)-1H-indol-4-amine (160 mg, 0.273 mmol) in 6 N aqueous HCl (10 mL). Stir for 2 hours at RT and concentrate. Purify by prep-HPLC to yield 2-[[6,7-dichloro-3-(1H-pyrazol-4-yl)-1H-indol-4-yl]amino]ethanol (62 mg, 0.19 mmol, 71% Yield). ES-MS (m/z): 311.0/313.0 (M+H). ¹H NMR (400 MHz, DMSO): 12.94 (br s, 1H), 11.42 (s, 1H), 7.72 (br s, 2H), 7.13 (d, J=2.3 Hz, 1H), 6.24 (s, 1H), 4.94 (br s, 1H), 4.71 (br s, 1H), 3.52 (t, J=5.4 Hz, 2H), 3.13 (t, J=5.0 Hz, 2H).

### SCHEME FOR EXAMPLES 63 & 64

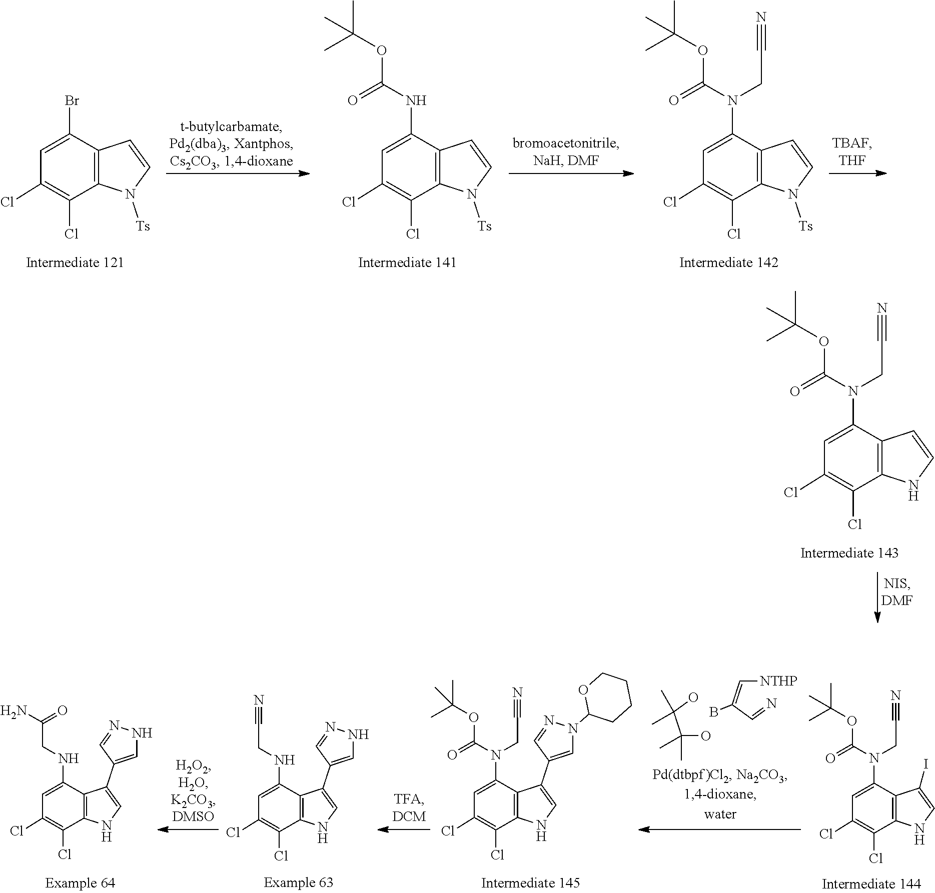

### Intermediate 141

### tert-Butyl N-[6, 7-dichloro-1-(p-tolylsulfonyl) indol-4-yl] carbamate

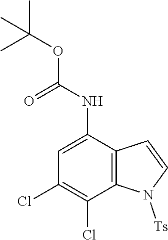 [SMILES: Cc1ccc(S(=O)(=O)n2ccc3c(NC(=O)OC(C)(C)C)cc(Cl)c(Cl)c32)cc1]

Dissolve 4-bromo-6, 7-dichloro-1-(p-tolylsulfonyl) indole (1.05 g, 2.25 mmol), tert-butyl carbamate (580 mg, 4.95 mmol), Pd₂(dba)₃ (200 mg, 0.218 mmol), Xantphos (250 mg, 0.432 mmol) and cesium carbonate (1.80 g, 5.52 mmol) in 1,4-dioxane (20 mL). Sparge with nitrogen and heat to 100° C. overnight. Dilute with water, extract with EtOAc, dry over anhydrous sodium sulfate, filter, and concentrate. Purify by flash column chromatography (EtOAc/petroleum ether) to yield tert-butyl N-[6, 7-dichloro-1-(p-tolylsulfonyl) indol-4-yl] carbamate (1.00 g, 1.98 mmol, 88% Yield).

### Intermediate 142

### tert-Butyl N-(cyanomethyl)-N-[6, 7-dichloro-1-(p-tolylsulfonyl) indol-4-yl] carbamate

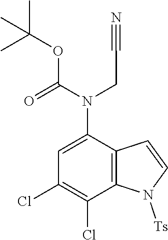 [SMILES: Cc1ccc(S(=O)(=O)n2ccc3c(N(CC#N)C(=O)OC(C)(C)C)cc(Cl)c(Cl)c32)cc1]

Dissolve tert-butyl N-[6, 7-dichloro-1-(p-tolylsulfonyl) indol-4-yl] carbamate (1.00 g, 1.98 mmol) in DMF (15 mL). Add NaH (400 mg, 10.0 mmol, 60% dispersion in mineral oil) at 0° C. and stir for 1 hour. Add bromoacetonitrile (1.20 g, 10.0 mmol) at 0° C. Stir at RT for 2 hours. Quench with water and extract with EtOAc, wash with brine, dry over anhydrous sodium sulfate, filter, and concentrate. Purify by flash column chromatography (EtOAc/petroleum ether) to yield tert-butyl N-(cyanomethyl)-N-[6, 7-dichloro-1-(p-tolylsulfonyl) indol-4-yl] carbamate (630 mg, 1.16 mmol, 59% Yield). ES-MS (m/z): 494.0/496.0 (M+H).

### Intermediate 143

### tert-Butyl N-(cyanomethyl)-N-(6, 7-dichloro-1H-indol-4-yl) carbamate

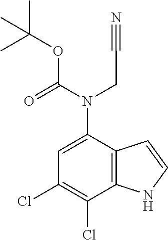 [SMILES: CC(C)(C)OC(=O)N(CC#N)c1cc(Cl)c(Cl)c2[nH]ccc12]

Dissolve tert-butyl N-(cyanomethyl)-N-[6, 7-dichloro-1-(p-tolylsulfonyl) indol-4-yl] carbamate (630 mg, 1.16 mmol) in THF (8 mL). Add TBAF (3.5 mL, 3.50 mmol, 1 M in THF) and stir at RT for 2 hours. Dilute with EtOAc, wash with saturated aqueous ammonium chloride, dry over anhydrous sodium sulfate, filter, and concentrate. Purify by flash column chromatography (EtOAc/petroleum ether) to yield tert-butyl N-(cyanomethyl)-N-(6, 7-dichloro-1H-indol-4-yl) carbamate (370 mg, 1.03 mmol, 89% Yield).

### Intermediate 144

### tert-Butyl N-(cyanomethyl)-N-(6, 7-dichloro-3-iodo-1H-indol-4-yl) carbamate

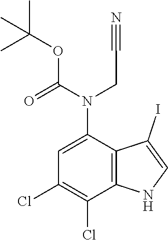 [SMILES: CC(C)(C)OC(=O)N(CC#N)c1cc(Cl)c(Cl)c2[nH]cc(I)c12]

Dissolve tert-butyl N-(cyanomethyl)-N-(6, 7-dichloro-1H-indol-4-yl) carbamate (370 mg, 1.03 mmol) in DMF (6 mL). Add NIS (262 mg, 1.14 mmol) and stir at RT for 2 hours. Dilute with EtOAc, wash with water, brine, dry over anhydrous sodium sulfate, filter and concentrate. Purify by flash column chromatography (EtOAc/petroleum ether) to yield tert-butyl N-(cyanomethyl)-N-(6, 7-dichloro-3-iodo-1H-indol-4-yl) carbamate (360 mg, 0.734 mmol, 71% Yield). ES-MS (m/z): 466.0/467.9 (M+H).

### Intermediate 145

### tert-Butyl N-(cyanomethyl)-N-[6, 7-dichloro-3-(1-tetrahydropyran-2-ylpyrazol-4-yl)-1H-indol-4-yl] carbamate

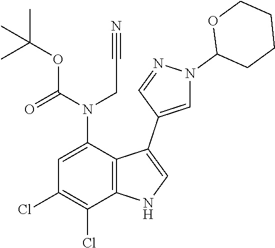 [SMILES: CC(C)(C)OC(=O)N(CC#N)c1cc(Cl)c(Cl)c2[nH]cc(-c3cnn(C4CCCCO4)c3)c12]

Dissolve tert-butyl N-(cyanomethyl)-N-(6,7-dichloro-3-iodo-1H-indol-4-yl)carbamate (330 mg, 0.673 mmol), 1-tetrahydropyran-2-yl-4-(4,4,5,5-tetramethyl-1,3,2-dioxaborolan-2-yl)pyrazole (281 mg, 1.01 mmol), Pd(dtbpf)Cl₂ (90 mg, 0.13 mmol) and Na₂CO₃ (215 mg, 2.02 mmol) in 1,4-dioxane (6 mL, 99.5 mass %) and water (1.5 mL). Sparge with nitrogen and heat to 90° C. for 2.5 hours. Concentrate and purify by flash column chromatography (EtOAc/petroleum ether) to yield tert-butyl N-(cyanomethyl)-N-[6, 7-dichloro-3-(1-tetrahydro pyran-2-ylpyrazol-4-yl)-1H-indol-4-yl] carbamate (230 mg, 0.399 mmol, 59% Yield). ES-MS (m/z): 490.2/492.2 (M+H).

### EXAMPLE 63

### 2-[[6, 7-Dichloro-3-(1H-pyrazol-4-yl)-1H-indol-4-yl]amino] acetonitrile

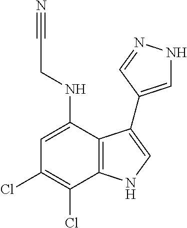 [SMILES: N#CCNc1cc(Cl)c(Cl)c2[nH]cc(-c3cn[nH]c3)c12]

Dissolve tert-butyl N-(cyanomethyl)-N-[6, 7-dichloro-3-(1-tetrahydropyran-2-ylpyrazol-4-yl)-1H-indol-4-yl] carbamate (230 mg, 0.399 mmol) in DCM (3 mL). Add TFA (1.5 mL) and stir at RT for 2 hours. Concentrate and neutralize with saturated aqueous sodium bicarbonate. Extract with EtOAc, dry over anhydrous sodium sulfate, filter, and concentrate. Purify by prep-HPLC to yield 2-[[6, 7-dichloro-3-(1H-pyrazol-4-yl)-1H-indol-4-yl]amino]acetonitrile (8 mg, 0.024 mmol) ES-MS (m/z): 306.2/308.2 (M+H). ¹H NMR (400 MHz, DMSO): 12.95 (br s, 1H), 11.61 (d, J=2.0 Hz, 1H), 7.69 (br s, 2H), 7.22 (d, J=2.6 Hz, 1H), 6.49 (s, 1H), 5.20 (t, J=6.8 Hz, 1H), 4.33 (d, J=6.8 Hz, 2H).

### EXAMPLE 64

### 2-[[6, 7-Dichloro-3-(1H-pyrazol-4-yl)-1H-indol-4-yl]amino] acetamide

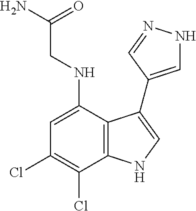 [SMILES: NC(=O)CNc1cc(Cl)c(Cl)c2[nH]cc(-c3cn[nH]c3)c12]

Dissolve 2-[[6, 7-dichloro-3-(1H-pyrazol-4-yl)-1H-indol-4-yl]amino] acetonitrile (140 mg, 0.220 mmol) and K₂CO₃ (170 mg, 1.23 mmol) in DMSO (3.5 mL). Add 30% aqueous H₂O₂ (140 mg) drop-wise at 0° C. Stir at RT for 2 hours. Dilute with water, extract with EtOAc, dry over anhydrous sodium sulfate, filter, and concentrate. Purify by prep-HPLC to yield 2-[[6, 7-dichloro-3-(1H-pyrazol-4-yl)-1H-indol-4-yl]amino]acetamide (30 mg, 0.093 mmol, 42% Yield). ES-MS (m/z): 324.0/326.0 (M+H). ¹H NMR (400 MHz, DMSO): 12.96 (br s, 1H), 11.47 (d, J=2.1 Hz, 1H), 7.96-7.54 (m, 2H), 7.51 (s, 1H), 7.16 (d, J=2.6 Hz, 1H), 7.14 (s, 1H), 6.05 (s, 1H), 5.28 (t, J=5.1 Hz, 1H), 3.67 (d, J=5.1 Hz, 2H).

### SCHEME FOR EXAMPLES 65 & 66

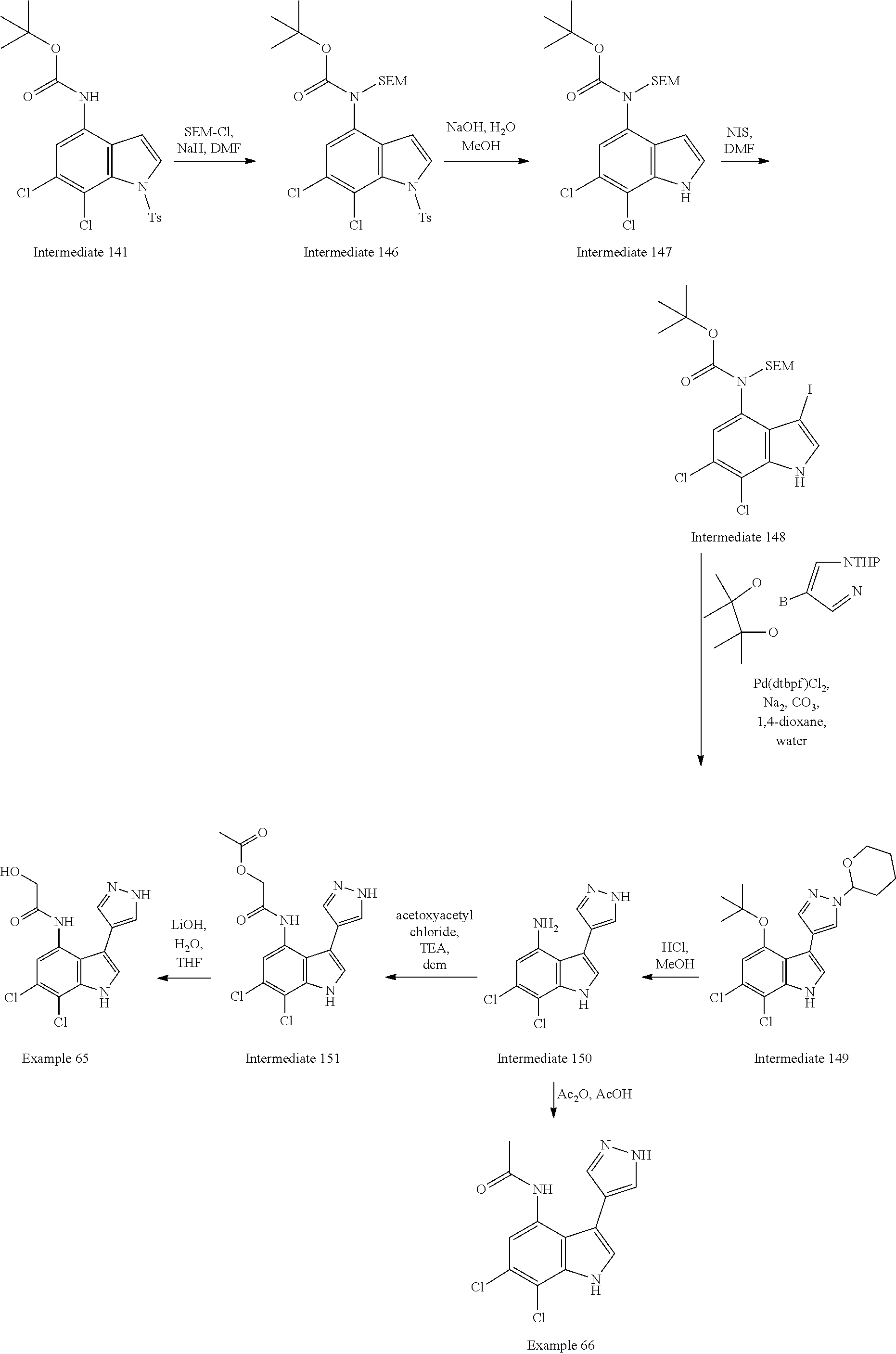

### Intermediate 146

### tert-Butyl N-[6,7-dichloro-1-(p-tolylsulfonyl)indol-4-yl]-N-(2-trimethylsilylethoxymethyl)carbamate

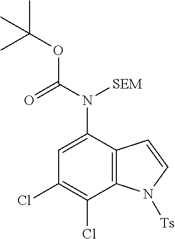 [SMILES: Cc1ccc(S(=O)(=O)n2ccc3c(N(COCC[Si](C)(C)C)C(=O)OC(C)(C)C)cc(Cl)c(Cl)c32)cc1]

Dissolve tert-butyl N-[6,7-dichloro-1-(p-tolylsulfonyl)indol-4-yl]carbamate (7.20 g, 14.0 mmol) in DMF (80 mL). Add NaH (1.70 g, 43.0 mmol, 60% dispersion in mineral oil) and stir for 1 hour at 0° C. Add SEM-Cl (3.80 mL, 21 mmol) and stir for 2 hours at 0° C. Quench with saturated aqueous ammonium chloride. Dilute with EtOAc, wash with brine, dry over anhydrous sodium sulfate, filter and concentrate to yield tert-butyl N-[6,7-dichloro-1-(p-tolyl sulfonyl)indol-4-yl]-N-(2-trimethylsilylethoxymethyl) carbamate (7.58 g, 12.6 mmol, 88% Yield).

### Intermediate 147

### tert-Butyl N-(6,7-dichloro-1H-indol-4-yl)-N-(2-trimethylsilylethoxymethyl)carbamate

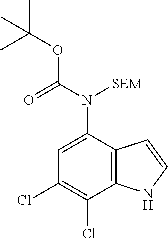 [SMILES: CC(C)(C)OC(=O)N(COCC[Si](C)(C)C)c1cc(Cl)c(Cl)c2[nH]ccc12]

Dissolve tert-butyl N-[6,7-dichloro-1-(p-tolylsulfonyl)indol-4-yl]-N-(2-trimethylsilylethoxy methyl)carbamate (7.58 g, 12.6 mmol) in water (18 mL) and MeOH (70 mL). Add NaOH (5.02 g, 126 mmol). Stir overnight at 60° C. Cool to RT, concentrate, and dilute with water. Extract with EtOAc, wash with brine, dry over sodium sulfate, filter, and concentrate to yield tert-butyl N-(6,7-dichloro-1H-indol-4-yl)-N-(2-trimethylsilylethoxymethyl)carbamate (4.48 g, 9.87 mmol, 79% Yield).

### Intermediate 148

### tert-Butyl N-(6,7-dichloro-3-iodo-1H-indol-4-yl)-N-(2-trimethylsilylethoxymethyl)carbamate

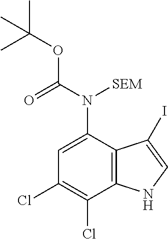 [SMILES: CC(C)(C)OC(=O)N(COCC[Si](C)(C)C)c1cc(Cl)c(Cl)c2[nH]cc(I)c12]

Dissolve tert-butyl N-(6,7-dichloro-1H-indol-4-yl)-N-(2-trimethylsilylethoxymethyl)carbamate (4.48 g, 9.87 mmol) in DMF (50 mL) and add NIS (2.49 g, 10.8 mmol). Stir for 2 hours at RT. Quench with water and stir for 5 min. Dilute with EtOAc, wash with brine, dry over anhydrous sodium sulfate, filter, and concentrate. Purify by flash column chromatography (EtOAc/petroleum) to yield tert-butyl N-(6,7-dichloro-3-iodo-1H-indol-4-yl)-N-(2-trimethylsilylethoxymethyl) carbamate (4.58 g, 7.81 mmol, 79% Yield).

### Intermediate 149

### tert-Butyl N-[6,7-dichloro-3-(1-tetrahydropyran-2-ylpyrazol-4-yl)-1H-indol-4-yl]-N-(2-trimethylsilylethoxymethyl)carbamate

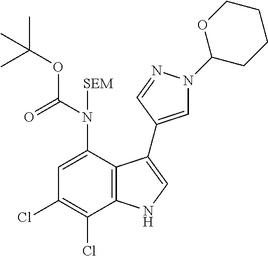 [SMILES: CC(C)(C)OC(=O)N(COCC[Si](C)(C)C)c1cc(Cl)c(Cl)c2[nH]cc(-c3cnn(C4CCCCO4)c3)c12]

Suspend tert-butyl N-(6,7-dichloro-3-iodo-1H-indol-4-yl)-N-(2-trimethylsilylethoxymethyl) carbamate (3.30 g, 5.60 mmol), 1-(tetrahydro-2H-pyran-2-yl)-4-(4, 4, 5, 5-tetramethyl-1, 3, 2-dioxaborolan-2-yl)-1H-pyrazole (460 mg, 1.60 mmol), Pd(dtbpf)Cl₂ (750 mg, 1.13 mmol)) and Na₂CO₃ (1.80 g, 17.0 mmol) in 1,4-dioxane (40 mL) and water (9 mL). Sparge with nitrogen and heat to 90° C. for 3 hours. Cool to RT, dilute with EtOAc, wash sequentially with saturated aqueous sodium bicarbonate and brine, dry over sodium sulfate, filter, and concentrate. Purify by flash column chromatography (EtOAc/petroleum ether) to yield tert-butyl N-[6,7-dichloro-3-(1-tetrahydropyran-2-ylpyrazol-4-yl)-1H-indol-4-yl]-N-(2-trimethylsilyl ethoxymethyl)carbamate (1.67 g, 2.73 mmol, 48% Yield). ES-MS (m/z): 524.8/526.8 (M+H, −tBu).

### Intermediate 150

### 6,7-Dichloro-3-(1H-pyrazol-4-yl)-1H-indol-4-amine hydrochloric acid salt

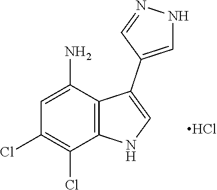 [SMILES: Nc1cc(Cl)c(Cl)c2[nH]cc(-c3cn[nH]c3)c12 ; Cl]

Suspend tert-butyl N-[6,7-dichloro-3-(1-tetrahydropyran-2-ylpyrazol-4-yl)-1H-indol-4-yl]-N-(2-trimethylsilylethoxymethyl)carbamate (1.67 g, 2.73 mmol) in MeOH (5 mL). Add 4 N HCl/MeOH (15 mL) and stir for 2.5 hours at RT. Concentrate and triturate with EtOAc, collecting by suction filtration, to yield 6,7-dichloro-3-(1H-pyrazol-4-yl)-1H-indol-4-amine (770 mg, 2.59 mmol, 95% Yield, HCl salt). ES-MS (m/z): 267.0/269.0 (M+H).

### Intermediate 151

### [2-[[6,7-Dichloro-3-(1H-pyrazol-4-yl)-1H-indol-4-yl]amino]-2-oxo-ethyl] acetate

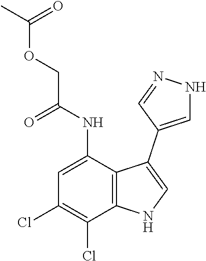 [SMILES: CC(=O)OCC(=O)Nc1cc(Cl)c(Cl)c2[nH]cc(-c3cn[nH]c3)c12]

Dissolve 6,7-dichloro-3-(1H-pyrazol-4-yl)-1H-indol-4-amine (200 mg, 0.670 mmol) and TEA (0.4 mL) in DCM (5 mL). Add acetoxyacetyl chloride (113 mg, 0.810 mmol). Stir for 2 hours at 0° C. Quench with water and stir vigorously for 15 min. Extract with DCM, wash with brine, dry over anhydrous sodium sulfate, filter, and concentrate to yield [2-[[6,7-dichloro-3-(1H-pyrazol-4-yl)-1H-indol-4-yl]amino]-2-oxo-ethyl] acetate (240 mg, 0.260 mmol, 39% Yield). ES-MS (m/z): 366.8/368.8 (M+H).

### EXAMPLE 65

### N-[6,7-Dichloro-3-(1H-pyrazol-4-yl)-1H-indol-4-yl]-2-hydroxy-acetamide

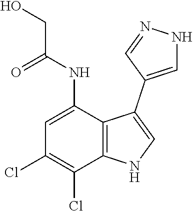 [SMILES: O=C(CO)Nc1cc(Cl)c(Cl)c2[nH]cc(-c3cn[nH]c3)c12]

Suspend [2-[[6,7-dichloro-3-(1H-pyrazol-4-yl)-1H-indol-4-yl]amino]-2-oxo-ethyl] acetate (180 mg, 0.240 mmol) in THF (5 mL) and water (1 mL). Add LiOH·H₂O (145, mg, 3.46 mmol) and stir for 2 hours at RT. Concentrate and dilute with EtOAc and water. Adjust to pH 8 with saturated aqueous NaHCO₃, extract with EtOAc, wash with brine, dry over anhydrous sodium sulfate, filter, and concentrate. Purify by prep-HPLC to yield N-[6,7-dichloro-3-(1H-pyrazol-4-yl)-1H-indol-4-yl]-2-hydroxy-acetamide (41.18 mg, 0.13 mmol, 53% Yield). ES-MS (m/z): 325.2/327.2 (M+H). ¹H NMR (400 MHz, DMSO): 12.91 (br s, 1H), 11.84 (br s, 1H), 9.28 (s, 1H), 8.15 (s, 1H), 7.96-7.48 (m, 2H), 7.35 (d, J=2.4 Hz, 1H), 5.56 (t, J=5.6 Hz, 1H), 3.82 (d, J=5.6 Hz, 2H).

### EXAMPLE 66

### N-[6, 7-Dichloro-3-(1H-pyrazol-4-yl)-1H-indol-4-yl]acetamide

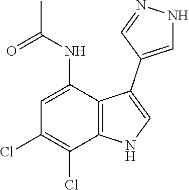 [SMILES: CC(=O)Nc1cc(Cl)c(Cl)c2[nH]cc(-c3cn[nH]c3)c12]

Dissolve 6, 7-dichloro-3-(1H-pyrazol-4-yl)-1H-indol-4-amine (20 mg, 0.071 mmol) in acetic acid (1 mL). Add acetic anhydride (0.01 mL, 0.1 mmol). Stir at RT for 2 hours. Concentrate and dilute with methanol (2 mL), then add K₂CO₃ (100 mg, 0.723 mmol). Stir at RT for 0.5 hour. Combine with another batch run at half scale for work up and purification. Dilute with water, extract EtOAc, wash with brine, dry over anhydrous sodium sulfate, filter and concentrate. Purify by prep-HPLC to yield N-[6,7-dichloro-3-(1H-pyrazol-4-yl)-1H-indol-4-yl]acetamide (19 mg, 0.060 mmol, 56% combined yield) as a white solid. ES-MS (m/z): 309.2/311.2 (M+H). ¹H NMR (400 MHz, DMSO): 12.90 (br s, 1H), 11.80 (s, 1H), 9.03 (s, 1H), 7.94-7.51 (m, 2H), 7.42-7.39 (m, 2H), 1.82 (s, 3H).

### SCHEME FOR EXAMPLE 67

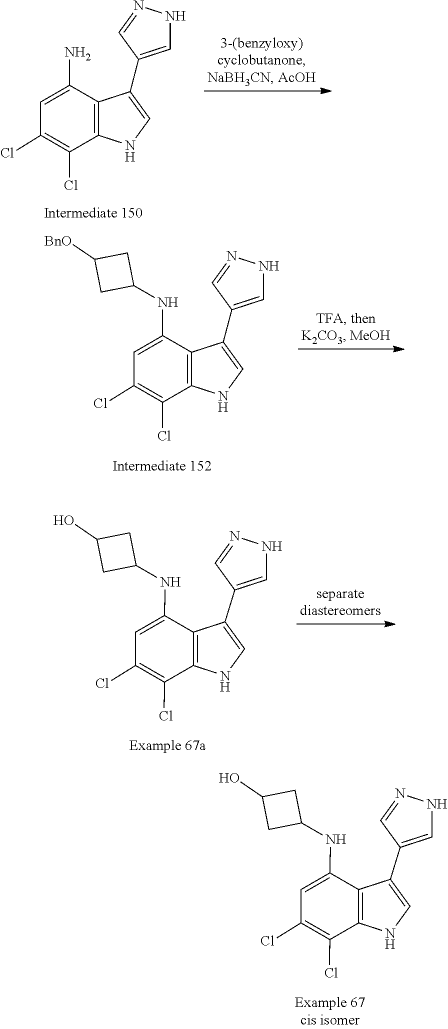 [SMILES: Nc1cc(Cl)c(Cl)c2[nH]cc(-c3cn[nH]c3)c12 ; Clc1cc(NC2CC(OCc3ccccc3)C2)c2c(-c3cn[nH]c3)c[nH]c2c1Cl ; OC1CC(Nc2cc(Cl)c(Cl)c3[nH]cc(-c4cn[nH]c4)c23)C1 ; OC1CC(Nc2cc(Cl)c(Cl)c3[nH]cc(-c4cn[nH]c4)c23)C1]

### Intermediate 152

### N-(3-Benzyloxycyclobutyl)-6, 7-dichloro-3-(1H-pyrazol-4-yl)-1H-indol-4-amine

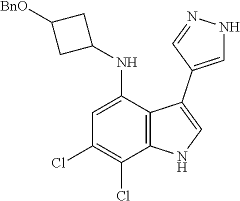 [SMILES: Clc1cc(NC2CC(OCc3ccccc3)C2)c2c(-c3cn[nH]c3)c[nH]c2c1Cl]

Dissolve 6, 7-dichloro-3-(1H-pyrazol-4-yl)-1H-indol-4-amine (145 mg, 0.489 mmol) in MeOH (6 mL). Add acetic acid (30 mg, 0.50 mmol) to adjust to pH 5 and stir 0.5 hour at RT. Add 3-(benzyloxy)cyclobutanone (267 mg, 1.47 mmol) to the mixture and stir 0.5 hour at RT. Add sodium cyanoborohydride (97 mg, 1.5 mmol) to the mixture and stir at RT overnight. Dilute with EtOAc, wash sequentially with saturated aqueous sodium bicarbonate and brine, dry over anhydrous sodium sulfate, filter, and concentrate. Purify by flash column chromatography (EtOAc/hexane) to yield N-(3-benzyloxycyclobutyl)-6, 7-dichloro-3-(1H-pyrazol-4-yl)-1H-indol-4-amine (100 mg, 0.206 mmol, 42% Yield). ES-MS (m/z): 427.0/429.1 (M+H).

### EXAMPLE 67A

### 3-((6, 7-Dichloro-3-(1H-pyrazol-4-yl)-1H-indol-4-yl)amino)cyclobutanol

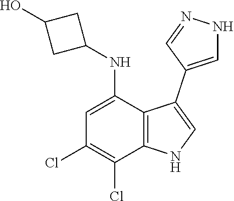 [SMILES: OC1CC(Nc2cc(Cl)c(Cl)c3[nH]cc(-c4cn[nH]c4)c23)C1]

Dissolve N-(3-benzyloxycyclobutyl)-6, 7-dichloro-3-(1H-pyrazol-4-yl)-1H-indol-4-amine (100 mg, 0.206 mmol) in TFA (2 mL) and stir at 90° C. overnight. Concentrate and dilute with MeOH (2 mL) and add potassium carbonate (57 mg, 0.41 mmol) and stir 2 hours at RT. Dilute with water, extract with EtOAc, dry over anhydrous sodium sulfate, filter and concentrate. Purify by prep-TLC (DCM:MeOH=10:1) to yield 3-((6, 7-dichloro-3-(1H-pyrazol-4-yl)-1H-indol-4-yl)amino)cyclobutanol (35 mg, 0.095 mmol, 46% Yield). ES-MS (m/z): 337.0/339.0 (M+H).

### EXAMPLE 67

### (cis)-3-((6, 7-Dichloro-3-(1H-pyrazol-4-yl)-1H-indol-4-yl)amino)cyclobutanol

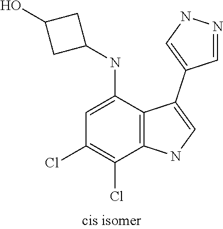

Purify the cis/trans mixture of 3-((6, 7-dichloro-3-(1H-pyrazol-4-yl)-1H-indol-4-yl)amino)cyclobutanol (55 mg, 0.15 mmol) by SFC (Instrument SFC-80, Method Column: DAICEL CHIRALPAK AD 250 mm*30 mm*10 um, Condition: 0.1% NH₄OH-EtOH. Begin B 30, End B 30, Flowrate: 70 ml/min, Injections 60) to yield (cis)-3-((6, 7-dichloro-3-(1H-pyrazol-4-yl)-1H-indol-4-yl)amino)cyclobutanol (14 mg, 0.041 mmol, 27% Yield). ES-MS (m/z): 337.0/339.0 (M+H). ¹H NMR (400 MHz, DMSO): 12.82 (br s, 1H), 11.46 (br s, 1H), 7.74 (s, 2H), 7.15 (s, 1H), 6.09 (s, 1H), 5.09 (br s, 1H), 4.67 (d, J=6.8 Hz, 1H), 3.85 (quintet, J=7.2 Hz, 1H), 3.42-3.34 (m, 1H), 2.75-2.64 (m, 2H), 1.49-1.39 (m, 2H).

### SCHEME FOR EXAMPLE 68

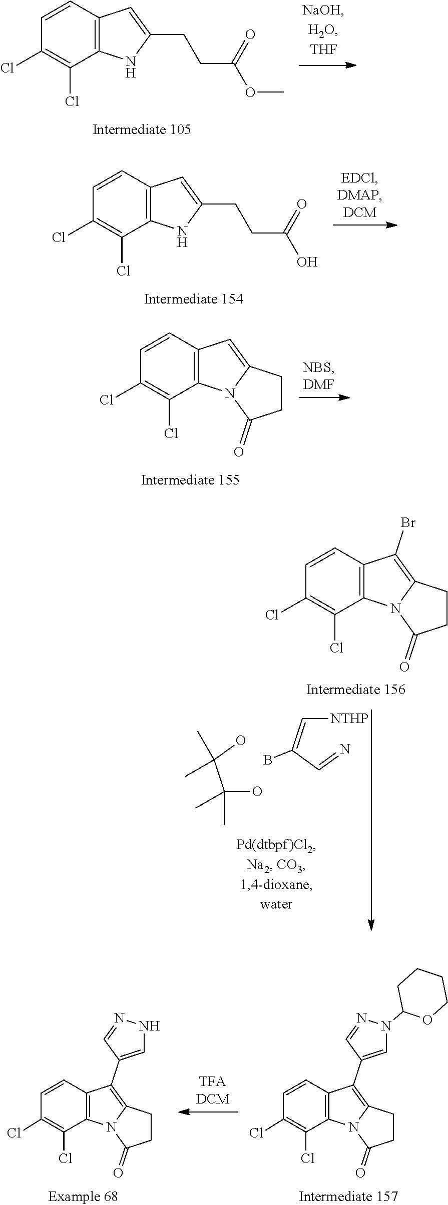

### Intermediate 154

### 3-(6, 7-Dichloro-1H-indol-2-yl) propanoic acid

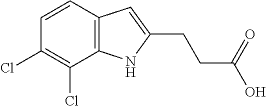 [SMILES: O=C(O)CCc1cc2ccc(Cl)c(Cl)c2[nH]1]

Dissolve methyl 3-(6, 7-dichloro-1H-indol-2-yl) propanoate (340 mg, 1.25 mmol) in water (5 mL) and THF (5 mL). Add NaOH (400 mg, 10.0 mmol). Stir at RT overnight. Acidify with 1N HCl to pH 4. Dilute with water, extract with EtOAc, dry over anhydrous sodium sulfate, filter and concentrate to yield 3-(6,7-dichloro-1H-indol-2-yl) propanoic acid (320 mg, 1.18 mmol, 94% Yield).

### Intermediate 155

### 7, 8-Dichloro-2, 3-dihydropyrrolo[1, 2-a] indol-1-one

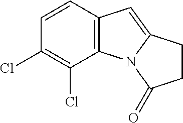 [SMILES: O=C1CCc2cc3ccc(Cl)c(Cl)c3n21]

Dissolve 3-(6, 7-dichloro-1H-indol-2-yl) propanoic acid (220 mg, 0.810 mmol) in DCM (5 mL) and add DMAP (198 mg, 1.62 mmol). Stir at RT for 10 minutes and add EDCI (314 mg, 1.62 mmol). Stir at 45° C. overnight. Dilute with water, extract with DCM, dry over anhydrous sodium sulfate, filter, and concentrate. Purify by flash column chromatography (EtOAc/petroleum ether) to yield 7, 8-dichloro-2, 3-dihydropyrrolo[1, 2-a] indol-1-one (220 mg, 0.916 mmol, 85% Yield).

### Intermediate 156

### 4-Bromo-7, 8-dichloro-2, 3-dihydropyrrolo[1, 2-a] indol-1-one

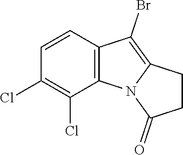 [SMILES: O=C1CCc2c(Br)c3ccc(Cl)c(Cl)c3n21]

Dissolve 7, 8-dichloro-2, 3-dihydropyrrolo[1, 2-a] indol-1-one (190 mg, 0.791 mmol) in DMF (3 mL) and add NBS (155 mg, 0.871 mmol). Stir at RT overnight. Dilute with EtOAc, wash with water and brine, dry over anhydrous sodium sulfate, filter, and concentrate. Purify by flash column chromatography (EtOAc/petroleum ether) to yield 4-bromo-7, 8-dichloro-2, 3-dihydropyrrolo[1, 2-a] indol-1-one (130 mg, 0.387 mmol, 49% Yield).

### Intermediate 157

### 7, 8-Dichloro-4-(1-tetrahydropyran-2-ylpyrazol-4-yl)-2,3-dihydropyrrolo[1, 2-a] indol-1-one

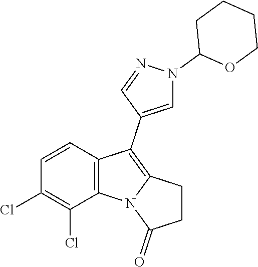 [SMILES: O=C1CCc2c(-c3cnn(C4CCCCO4)c3)c3ccc(Cl)c(Cl)c3n21]

Suspend 4-bromo-7,8-dichloro-2,3-dihydropyrrolo[1,2-a]indol-1-one (100 mg, 0.298 mmol), 1-(tetrahydro-2h-pyran-2-yl)-4-(4,4,5,5-tetramethyl-1,3,2-dioxaborolan-2-yl)-1h-pyrazole (97 mg, 0.34 mmol), Pd(dtbpf)Cl₂ (20 mg, 0.030 mmol) and sodium carbonate (63 mg, 0.59 mmol) in 1,4-dioxane (3 mL) and water (0.5 mL). Sparge with nitrogen and heat to 90° C. for 3 hours. Cool to RT and concentrate. Purify by flash column chromatography (EtOAc/petroleum ether) to yield 7, 8-dichloro-4-(1-tetrahydropyran-2-ylpyrazol-4-yl)-2,3-dihydropyrrolo[1, 2-a] indol-1-one (30 mg, 0.062 mmol, 16% Yield). ES-MS (m/z): 390.3/391.9 (M+H).

### EXAMPLE 68

### 7, 8-Dichloro-4-(1H-pyrazol-4-yl)-2,3-dihydropyrrolo[1, 2-a] indol-1-one

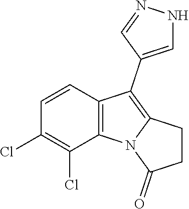 [SMILES: O=C1CCc2c(-c3cn[nH]c3)c3ccc(Cl)c(Cl)c3n21]

Dissolve 7, 8-dichloro-4-(1-tetrahydropyran-2-ylpyrazol-4-yl)-2,3-dihydropyrrolo [1, 2-a] indol-1-one (30 mg, 0.062 mmol) in DCM (1 mL) and add TFA (0.5 mL). Stir at RT for 2 hours and concentrate. Purify by prep-HPLC to yield 7, 8-dichloro-4-(1H-pyrazol-4-yl)-2,3-dihydropyrrolo[1,2-a]indol-1-one (6.4 mg, 0.021 mmol, 34% Yield). ES-MS (m/z): 306.2/308.2 (M+H). ¹H NMR (400 MHz, DMSO): 13.15 (br s, 1H), 8.22 (br s, 1H), 7.91 (br s, 1H), 7.80 (d, J=8.5 Hz, 1H), 7.54 (d, J=8.5 Hz, 1H), 3.29-3.22 (m, 2H), 3.21-3.13 (m, 2H).

### SCHEME FOR EXAMPLE 69

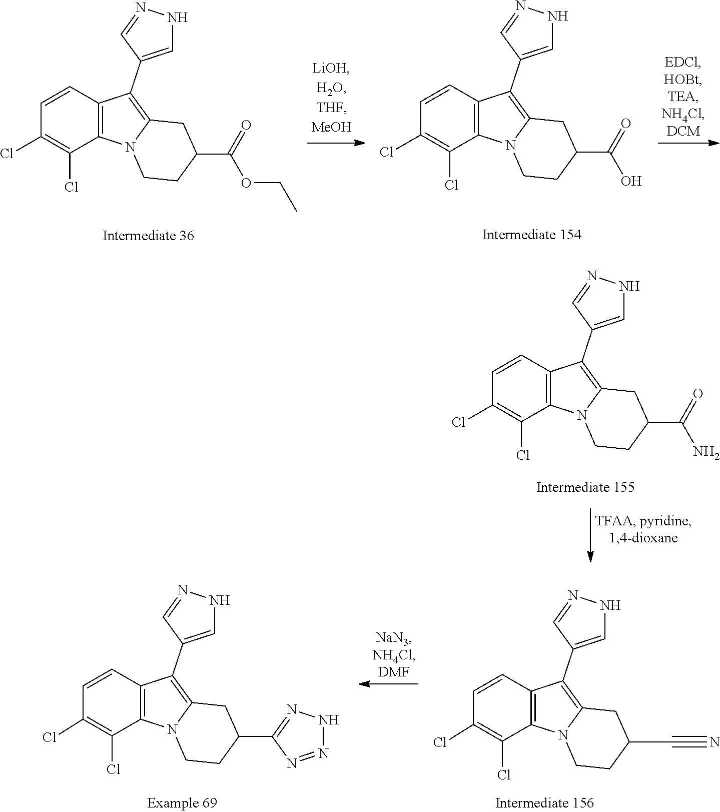 [SMILES: CCOC(=O)C1CCn2c(c(-c3cn[nH]c3)c3ccc(Cl)c(Cl)c32)C1 ; O=C(O)C1CCn2c(c(-c3cn[nH]c3)c3ccc(Cl)c(Cl)c32)C1 ; NC(=O)C1CCn2c(c(-c3cn[nH]c3)c3ccc(Cl)c(Cl)c32)C1 ; Clc1ccc2c(-c3cn[nH]c3)c3n(c2c1Cl)CCC(c1nn[nH]n1)C3 ; N#CC1CCn2c(c(-c3cn[nH]c3)c3ccc(Cl)c(Cl)c32)C1]

### Intermediate 154

### 3,4-Dichloro-10-(1H-pyrazol-4-yl)-6,7,8,9-tetrahydropyrido[1,2-a]indole-8-carboxylic acid

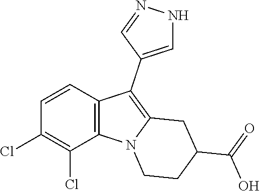 [SMILES: O=C(O)C1CCn2c(c(-c3cn[nH]c3)c3ccc(Cl)c(Cl)c32)C1]

Dissolve ethyl 3,4-dichloro-10-(1H-pyrazol-4-yl)-6,7,8,9-tetrahydropyrido[1,2-a] indole-8-carboxylate (1.48 g, 2.02 mmol, 33% purity) in THF (15 mL), water (5 mL) and methanol (10 mL) and add LiOH·H₂O (275 mg, 6.55 mmol) at RT. Stir for 1 hour at 50° C. Add water and wash with MTBE. Adjust the pH of aqueous layer to 3 with 1 N aqueous HCl and extract with EtOAc. Dry over anhydrous sodium sulfate, filter, and concentrate. Triturate with ACN to yield 3,4-dichloro-10-(1H-pyrazol-4-yl)-6,7,8,9-tetrahydropyrido[1,2-a]indole-8-carboxylic acid (174 mg, 0.432 mmol, 33% Yield). ES-MS (m/z): 349.6/351.6 (M+1).

### Intermediate 155

### 3,4-Dichloro-10-(1H-pyrazol-4-yl)-6,7,8,9-tetrahydropyrido[1,2-a]indole-8-carboxamide

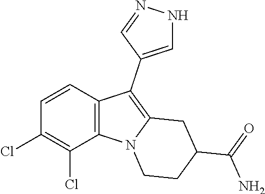 [SMILES: NC(=O)C1CCn2c(c(-c3cn[nH]c3)c3ccc(Cl)c(Cl)c32)C1]

Suspend 3,4-dichloro-10-(1H-pyrazol-4-yl)-6,7,8,9-tetrahydropyrido[1,2-a]indole-8-carboxylic acid (170 mg, 0.422 mmol), EDCI (100 mg, 0.511 mmol), HOBt (75 mg, 0.53 mmol) and TEA (0.3 mL, 2 mmol) in DMF (3 mL) and stir at RT for 15 minutes. Add NH₄Cl (45 mg, 0.84 mmol) and stir at RT overnight. Add water, extract with EtOAc, dry over anhydrous sodium sulfate, filter, and concentrate. Purify by trituration with ACN to yield 3,4-dichloro-10-(1H-pyrazol-4-yl)-6,7,8,9-tetrahydropyrido[1,2-a]indole-8-carboxamide (85 mg, 0.23 mmol, 52% Yield). ES-MS (m/z): 349.1/351.0 (M+H).

### Intermediate 156

### 3,4-Dichloro-10-(1H-pyrazol-4-yl)-6,7,8,9-tetrahydropyrido[1,2-a]indole-8-carbonitrile

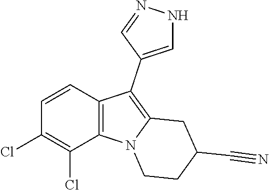 [SMILES: N#CC1CCn2c(c(-c3cn[nH]c3)c3ccc(Cl)c(Cl)c32)C1]

Suspend 3,4-dichloro-10-(1H-pyrazol-4-yl)-6,7,8,9-tetrahydropyrido[1,2-a]indole-8-carboxamide (85 mg, 0.23 mmol), TFAA (0.5 mL) and pyridine (0.050 mL) in 1,4-dioxane and stir at RT for 2 hours. Add 1 N aqueous HCl and extract with EtOAc. Dry over anhydrous sodium sulfate, filter, and concentrate. Purify by trituration with petroleum ether to yield 3,4-dichloro-10-(1H-pyrazol-4-yl)-6,7,8,9-tetrahydropyrido[1,2-a]indole-8-carbonitrile (88 mg, 0.23 mmol, 99% Yield). ES-MS (m/z): 331.1/333.1 (M+H).

### EXAMPLE 69

### 3,4-Dichloro-10-(1H-pyrazol-4-yl)-8-(1H-tetrazol-5-yl)-6,7,8,9-tetrahydropyrido[1,2-a]indole

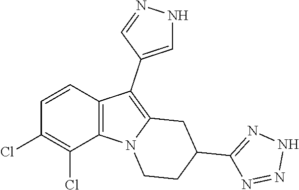 [SMILES: Clc1ccc2c(-c3cn[nH]c3)c3n(c2c1Cl)CCC(c1nn[nH]n1)C3]

Suspend 3, 4-dichloro-10-(1H-pyrazol-4-yl)-6, 7, 8, 9-tetrahydropyrido[1,2-a]indole-8-carbonitrile (88 mg, 0.23 mmol) in DMF (2 mL). Add NH₄Cl (40 mg, 0.75 mmol) and NaN₃ (130 mg, 1.98 mmol). Stir at 80° C. overnight. Dilute with water and extract with EtOAc. Wash with brine twice, dry over anhydrous sodium sulfate, filter, and concentrate. Purify by prep-HPLC to yield 3,4-dichloro-10-(1H-pyrazol-4-yl)-8-(1H-tetrazol-5-yl)-6, 7, 8, 9-tetrahydropyrido[1,2-a]indole (27 mg, 0.072 mmol, 32% Yield). ES-MS (m/z): 374.3/376.2 (M+H). ¹H NMR (400 MHz, DMSO): 13.04 (br s, 1H), 7.87 (br s, 2H), 7.59 (d, J=8.5 Hz, 1H), 7.25 (d, J=8.4 Hz, 1H), 5.05 (dt, J=12.2, 4.3 Hz, 1H), 4.51 (td, J=11.6, 4.8 Hz, 1H), 3.25 (dd, J=16.4, 10.5 Hz, 2H), 2.63-2.55 (m, 2H), 2.36-2.25 (m, 1H).

### SCHEME FOR EXAMPLE 71

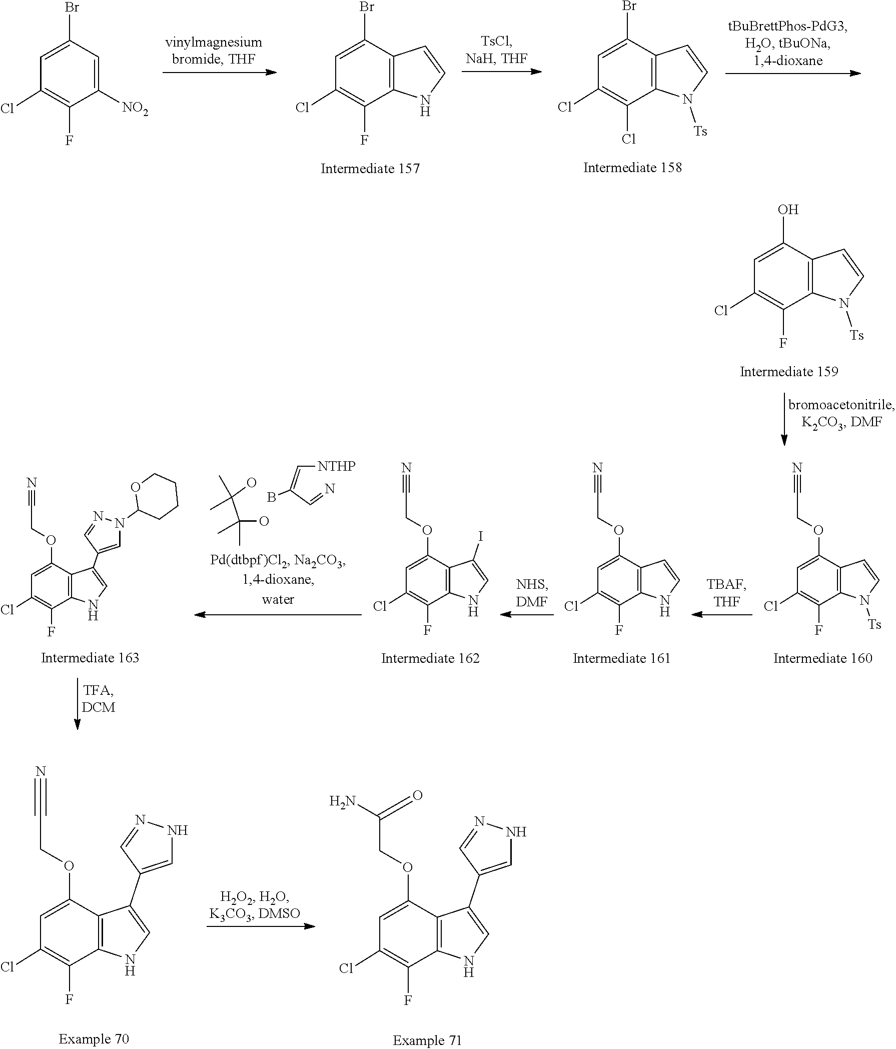

### Intermediate 157

### 4-Bromo-6-chloro-7-fluoro-1H-indole

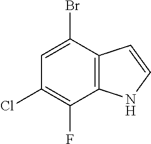 [SMILES: Fc1c(Cl)cc(Br)c2cc[nH]c12]

Dissolve 5-bromo-1-chloro-2-fluoro-3-nitro-benzene (19.1 g, 67.6 mmol) in THF (100 mL). Cool to −60° C. under nitrogen and add vinylmagnesium bromide solution (270 mL, 270 mmol, 1 M in THF) drop-wise via an addition funnel. Stir at low temperature for 2 hours. Slowly quench with saturated aqueous NH₄Cl at 0° C. Add water and extract with EtOAc, wash with brine, dry over anhydrous sodium sulfate, filter, and concentrate. Purify by flash column chromatography (EtOAc/petroleum ether) to yield 4-bromo-6-chloro-7-fluoro-1H-indole (2.10 g, 7.60 mmol, 11% Yield).

### Intermediate 158

### 4-Bromo-6-chloro-7-fluoro-1-tosyl-1H-indole

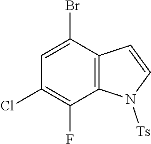 [SMILES: Cc1ccc(S(=O)(=O)n2ccc3c(Br)cc(Cl)c(F)c32)cc1]

Suspend NaH (920 mg, 23.0 mmol, 60% dispersion in mineral oil) in DMF (15 mL). Add a solution of 4-bromo-6-chloro-7-fluoro-1H-indole (2.10 g, 7.61 mmol) in DMF (10 mL) dropwise at 0° C. Stir at 0° C. for 1 hour, then add 4-methylbenzenesulfonyl chloride (3.63 g, 19.0 mmol) portion-wise at 0° C. Stir at RT for 3 hours. Quench with ice-water and extract with EtOAc. Wash with brine, dry over anhydrous sodium sulfate, filter, and concentrate. Purify by flash column chromatography (EtOAc/petroleum ether) to yield 4-bromo-6-chloro-7-fluoro-1-tosyl-1H-indole (2.70 g, 6.03 mmol, 80% Yield).

### Intermediate 159

### 6-Chloro-7-fluoro-1-(p-tolylsulfonyl) indol-4-ol

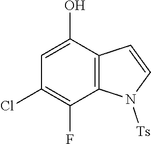 [SMILES: Cc1ccc(S(=O)(=O)n2ccc3c(O)cc(Cl)c(F)c32)cc1]

Suspend 4-bromo-6-chloro-7-fluoro-1-(p-tolylsulfonyl)indole (2.70 g, 6.03 mmol), sodium tert-butoxide (1.49 g, 15.0 mmol) and tert-BuBrettPhos-Pd-G₃ (530 mg, 0.608 mmol) in 1,4-dioxane (50 mL) and water (13 mL). Sparge with nitrogen and heat to 65° C. for 3 hours. Concentrate and acidify to pH 2 with 1 N aqueous HCl. Extract with EtOAc, dry over anhydrous sodium sulfate, filter, and concentrate. Purify by flash column chromatography (EtOAc/petroleum ether) to yield 6-chloro-7-fluoro-1-(p-tolyl sulfonyl) indol-4-ol (1.70 g, 4.75 mmol, 79% Yield).

### Intermediate 160

### 2-[6-Chloro-7-fluoro-1-(p-tolyl sulfonyl) indol-4-yl] oxyacetonitrile

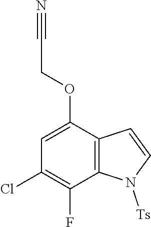 [SMILES: Cc1ccc(S(=O)(=O)n2ccc3c(OCC#N)cc(Cl)c(F)c32)cc1]

Dissolve 6-chloro-7-fluoro-1-(p-tolylsulfonyl) indol-4-ol (1.70 g, 4.75 mmol) in DMF (25 mL). Add bromoacetonitrile (1.14 g, 9.50 mmol) and potassium carbonate (860 mg, 6.22 mmol). Stir at RT for 2 hours. Dilute with water and extract with EtOAc. Dry over anhydrous sodium sulfate, filter, and concentrate. Purify by flash column chromatography (EtOAc/petroleum ether) to yield 2-[6-chloro-7-fluoro-1-(p-tolylsulfonyl) indol-4-yl] oxyacetonitrile (1.80 g, 4.51 mmol, 95% Yield). ES-MS (m/z): (M+H).

### Intermediate 161

### 2-[(6-Chloro-7-fluoro-1H-indol-4-yl) oxy] acetonitrile

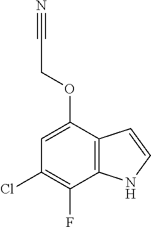 [SMILES: N#CCOc1cc(Cl)c(F)c2[nH]ccc12]

Dissolve 2-[6-chloro-7-fluoro-1-(p-tolyl sulfonyl) indol-4-yl] oxyacetonitrile (1.80 g, 4.50 mmol) in THF (25 mL). Add TBAF (14 mL, 1 M in THF) and stir at RT for 2 hours. Dilute with EtOAc, wash with saturated aqueous NH₄Cl, dry over anhydrous sodium sulfate, filter and concentrate to yield 2-[(6-chloro-7-fluoro-1H-indol-4-yl)oxy]acetonitrile (1.05 g, 4.44 mmol, 98% Yield). ES-MS (m/z): 225.0/226.9 (M+H).

### Intermediate 162

### 2-[(6-Chloro-7-fluoro-3-iodo-1H-indol-4-yl) oxy] acetonitrile

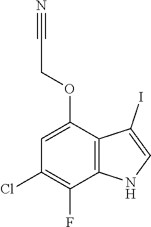 [SMILES: N#CCOc1cc(Cl)c(F)c2[nH]cc(I)c12]

Dissolve 2-[(6-chloro-7-fluoro-1H-indol-4-yl) oxy] acetonitrile (110 mg, 0.465 mmol) in DMF (3 mL). Add NIS (117 mg, 0.510 mmol) and stir at RT for 1 hour. Dilute with EtOAc, wash with saturated aqueous sodium sulfite, and brine. Dry over anhydrous sodium sulfate, filter, and concentrate. Purify by flash column chromatography (EtOAc/petroleum ether) to yield 2-[(6-chloro-7-fluoro-3-iodo-1H-indol-4-yl) oxy] acetonitrile (140 mg, 0.379 mmol, 82% Yield) as a yellow solid.

### Intermediate 163

### 2-[[6-Chloro-7-fluoro-3-(1-tetrahydropyran-2-ylpyrazol-4-yl)-1h-indol-4-yl] oxy] acetonitrile

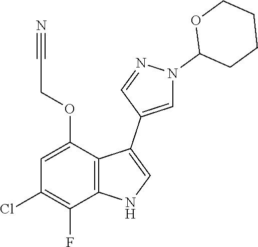 [SMILES: N#CCOc1cc(Cl)c(F)c2[nH]cc(-c3cnn(C4CCCCO4)c3)c12]

Dissolve 2-[(6-chloro-7-fluoro-3-iodo-1H-indol-4-yl)oxy]acetonitrile (140 mg, 0.379 mmol), 1-tetrahydropyran-2-yl-4-(4,4,5,5-tetramethyl-1,3,2-dioxaborolan-2-yl)pyrazole (158 mg, 0.569 mmol), Pd(dtbpf)Cl₂ (25 mg, 0.038 mmol) and Na₂CO₃ (121 mg, 1.14 mmol) in 1,4-dioxane (3 mL) and water (0.75 mL). Sparge with nitrogen and heat to 90° C. for 1 hour. Concentrate and purify by flash column chromatography (EtOAc/petroleum ether) to yield 2-[[6-chloro-7-fluoro-3-(1-tetrahydropyran-2-ylpyrazol-4-yl)-1H-indol-4-yl] oxy] acetonitrile (100 mg, 0.189 mmol, 50% Yield). ES-MS (m/z): 375.1/377.1 (M+H).

### EXAMPLE 70

### 2-[[6-Chloro-7-fluoro-3-(1H-pyrazol-4-yl)-1H-indol-4-yl] oxy] acetonitrile

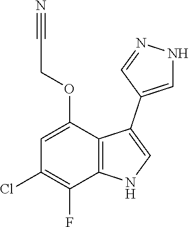 [SMILES: N#CCOc1cc(Cl)c(F)c2[nH]cc(-c3cn[nH]c3)c12]

Dissolve 2-[[6-chloro-7-fluoro-3-(1-tetrahydropyran-2-ylpyrazol-4-yl)-1H-indol-4-yl]oxy]aceto nitrile (100 mg, 0.189 mmol) in DCM (3 mL). Add TFA (1 mL) and stir at RT for 2 hours. Concentrate and purify by prep-HPLC to yield 2-[[6-chloro-7-fluoro-3-(1H-pyrazol-4-yl)-1H-indol-4-yl]oxy]acetonitrile (19 mg, 0.064 mmol, 34% Yield). ES-MS (m/z): 291.0/293.0 (M+H). ¹H NMR (400 MHz, DMSO): 12.75 (br s, 1H), 12.04 (d, J=1.6 Hz, 1H), 7.82 (s, 2H), 7.56 (d, J=2.4 Hz, 1H), 6.78 (d, J=5.0 Hz, 1H), 5.27 (s, 2H).

### EXAMPLE 71

### 2-[[6-Chloro-7-fluoro-3-(1H-pyrazol-4-yl)-1H-indol-4-yl] oxy] acetamide

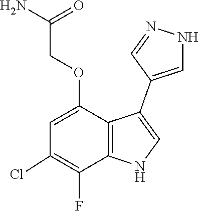 [SMILES: NC(=O)COc1cc(Cl)c(F)c2[nH]cc(-c3cn[nH]c3)c12]

Dissolve 2-[[6-chloro-7-fluoro-3-(1H-pyrazol-4-yl)-1H-indol-4-yl]oxy]acetonitrile (110 mg, 0.360 mmol) and K₂CO₃ (124 mg, 0.897 mmol) in DMSO (2.5 mL). Add 30% aqueous H₂O₂ (160 mg, 1.41 mmol) dropwise at 0° C. Stir at RT for 2 hours and filter. Purify the filtrate by prep-HPLC to yield 2-[[6-chloro-7-fluoro-3-(1H-pyrazol-4-yl)-1H-indol-4-yl]oxy]acetamide (54 mg, 0.17 mmol, 49% Yield). ES-MS (m/z): 309.0/311.0 (M+H). ¹H NMR (400 MHz, DMSO): 12.69 (br s, 1H), 11.90 (d, J=1.6 Hz, 1H), 8.26-7.65 (m, 2H), 7.50 (d, J=2.4 Hz, 1H), 7.40 (s, 1H), 7.23 (s, 1H), 6.46 (d, J=5.1 Hz, 1H), 4.54 (s, 2H).

### SCHEME FOR EXAMPLE 72

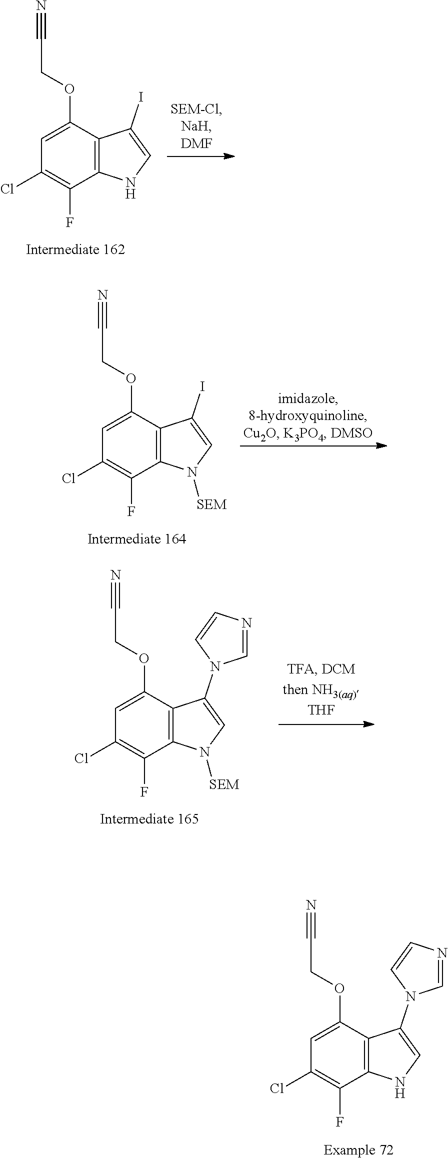 [SMILES: N#CCOc1cc(Cl)c(F)c2[nH]cc(I)c12 ; C[Si](C)(C)CCOCn1cc(I)c2c(OCC#N)cc(Cl)c(F)c21 ; C[Si](C)(C)CCOCn1cc(-n2ccnc2)c2c(OCC#N)cc(Cl)c(F)c21 ; N#CCOc1cc(Cl)c(F)c2[nH]cc(-n3ccnc3)c12]

### Intermediate 164

### 2-[6-Chloro-7-fluoro-3-iodo-1-(2-trimethylsilylethoxymethyl) indol-4-yl] oxyacetonitrile

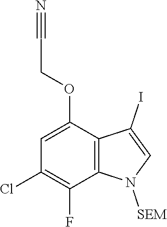 [SMILES: C[Si](C)(C)CCOCn1cc(I)c2c(OCC#N)cc(Cl)c(F)c21]

Dissolve 2-[(6-chloro-7-fluoro-3-iodo-1H-indol-4-yl) oxy]acetonitrile (300 mg, 0.813 mmol) in DMF (5 mL). Add NaH (50 mg, 1.3 mmol, 60% in mineral oil) at 0° C. Stir at 0° C. for 1 hour. Add SEM-Cl (210 mg, 1.23 mmol) dropwise at 0° C. Stir at RT for 2 hours. Quench with water, extract with EtOAc, dry over anhydrous sodium sulfate, filter, and concentrate. Purify by flash column chromatography (EtOAc/petroleum ether) to yield 2-[6-chloro-7-fluoro-3-iodo-1-(2-trimethylsilylethoxymethyl) indol-4-yl] oxyacetonitrile (270 mg, 0.561 mmol, 69% Yield).

### Intermediate 165

### 2-[6-Chloro-7-fluoro-3-imidazol-1-yl-1-(2-trimethylsilylethoxymethyl) indol-4-yl] oxyacetonitrile

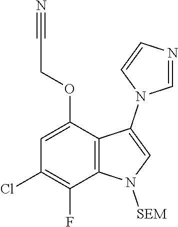 [SMILES: C[Si](C)(C)CCOCn1cc(-n2ccnc2)c2c(OCC#N)cc(Cl)c(F)c21]

Dissolve 2-[6-chloro-7-fluoro-3-iodo-1-(2-trimethylsilylethoxymethyl)indol-4-yl]oxyacetonitrile (30 mg, 0.059 mmol), imidazole (13 mg, 0.19 mmol), CuI (3 mg, 0.02 mmol), 8-hydroxyquinoline (4 mg, 0.03 mmol) and K₃PO₄ (41 mg, 0.19 mmol) in DMSO (1.5 mL). Sparge with nitrogen and heat to 100° C. overnight. Combine four parallel reaction mixtures of the same scale, dilute with water, extract with EtOAc, wash with brine, dry over sodium sulfate, filter, and concentrate. Purify by flash column chromatography (EtOAc/petroleum ether) to yield 2-[6-chloro-7-fluoro-3-imidazol-1-yl-1-(2-trimethylsilylethoxymethyl) indol-4-yl] oxyacetonitrile (55 mg, 0.11 mmol, 45% Yield). ES-MS (m/z): 420.9/422.8 (M+H).

### EXAMPLE 72

### 2-[(6-Chloro-7-fluoro-3-imidazol-1-yl-1H-indol-4-yl)oxy]acetonitrile

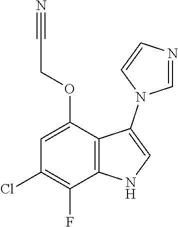 [SMILES: N#CCOc1cc(Cl)c(F)c2[nH]cc(-n3ccnc3)c12]

Dissolve 2-[6-chloro-7-fluoro-3-imidazol-1-yl-1-(2-trimethylsilylethoxymethyl)indol-4-yl]oxy acetonitrile (50 mg, 0.097 mmol) in DCM (3 mL). Add TFA (1 mL) and stir at RT for 1.5 hours. Concentrate and dilute with THF (3 mL), then add 28% aqueous ammonia (0.5 mL). Stir at RT for 2 hours. Concentrate and purify by prep-HPLC to yield 2-[(6-chloro-7-fluoro-3-imidazol-1-yl-1H-indol-4-yl)oxy]acetonitrile (15 mg, 0.051 mmol, 52% Yield). ES-MS (m/z): 291.0/293.0 (M+H). ¹H NMR (400 MHz, DMSO): 12.45 (s, 1H), 8.03 (br s, 1H), 7.80 (d, J=2.8 Hz, 1H), 7.47 (br s, 1H), 7.15 (br s, 1H), 6.91 (d, J=4.9 Hz, 1H), 5.14 (s, 2H).

### SCHEME FOR EXAMPLE 73

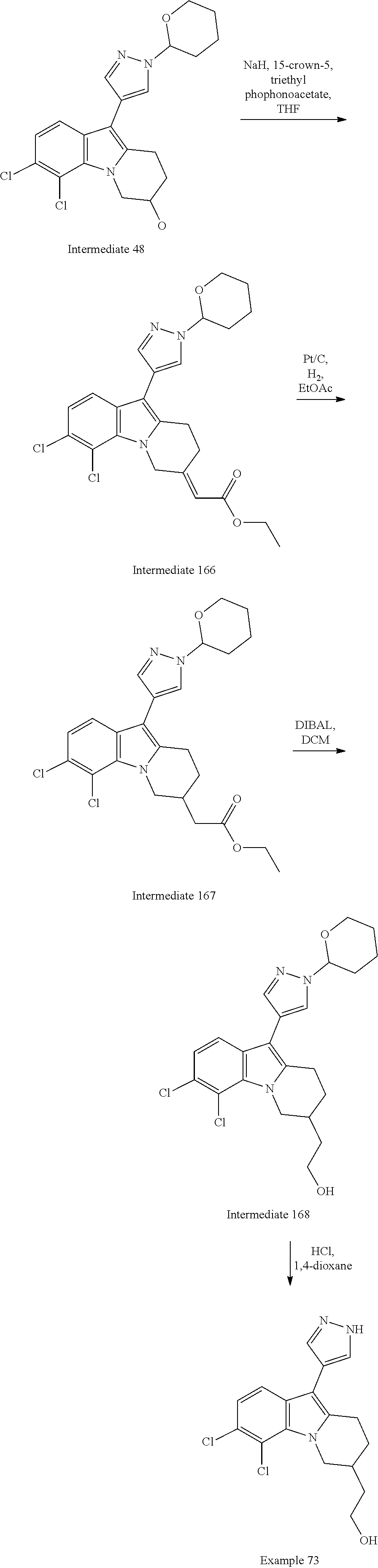 [SMILES: OC1CCc2c(-c3cnn(C4CCCCO4)c3)c3ccc(Cl)c(Cl)c3n2C1 ; CCOC(=O)/C=C1\CCc2c(-c3cnn(C4CCCCO4)c3)c3ccc(Cl)c(Cl)c3n2C1 ; CCOC(=O)CC1CCc2c(-c3cnn(C4CCCCO4)c3)c3ccc(Cl)c(Cl)c3n2C1 ; OCCC1CCc2c(-c3cnn(C4CCCCO4)c3)c3ccc(Cl)c(Cl)c3n2C1 ; OCCC1CCc2c(-c3cn[nH]c3)c3ccc(Cl)c(Cl)c3n2C1]

### Intermediate 166

### Ethyl (2E)-2-[3,4-dichloro-10-(1-tetrahydropyran-2-ylpyrazol-4-yl)-8, 9-dihydro-6H-pyrido[1, 2-a] indol-7-ylidene] acetate

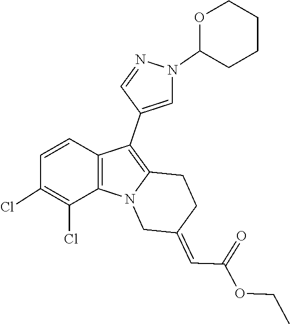 [SMILES: CCOC(=O)/C=C1\CCc2c(-c3cnn(C4CCCCO4)c3)c3ccc(Cl)c(Cl)c3n2C1]

Suspend NaH (32 mg, 0.80 mmol, 60% dispersion in mineral oil) and 15-crown-5 (20 mg, 0.091 mmol) in THF (2 mL). Add triethyl phosphonoacetate (181 mg, 0.799 mmol) in THF (1 mL) dropwise at 0° C. Stir at RT for 1 hour. Cool to 0° C. and add 3, 4-dichloro-10-(1-tetrahydropyran-2-ylpyrazol-4-yl)-8, 9-dihydro-6H-pyrido[1, 2-a]indol-7-one (200 mg, 0.445 mmol) in THF (1 mL) dropwise at 0° C. Stir at RT for 3 hours. Quench with water, extract with EtOAc, dry over anhydrous sodium sulfate, filter, and concentrate. Purify by flash column chromatography (EtOAc/petroleum ether) to yield ethyl (2E)-2-[3,4-dichloro-10-(1-tetrahydropyran-2-ylpyrazol-4-yl)-8, 9-dihydro-6H-pyrido[1, 2-a] indol-7-ylidene] acetate (190 mg, 0.380 mmol, 85% Yield).

### Intermediate 167

### Ethyl 2-[3,4-dichloro-10-(1-tetrahydropyran-2-ylpyrazol-4-yl)-6,7,8,9-tetrahydropyrido [1,2-a]indol-7-yl]acetate

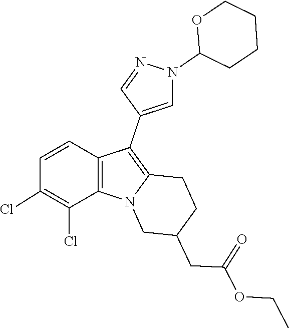 [SMILES: CCOC(=O)CC1CCc2c(-c3cnn(C4CCCCO4)c3)c3ccc(Cl)c(Cl)c3n2C1]

Dissolve ethyl (2E)-2-[3,4-dichloro-10-(1-tetrahydropyran-2-ylpyrazol-4-yl)-8, 9-dihydro-6H-pyrido[1, 2-a] indol-7-ylidene] acetate (190 mg, 0.380 mmol) in EtOAc (8 mL). Add 5% Pt/C (190 mg). Purge with hydrogen gas and stir at RT overnight (50 psi). Filter through a pad of diatomaceous earth. Add additional 5% Pt/C (190 mg) and stir at RT overnight under H₂ (50 psi). Concentrate and purify by flash column chromatography (EtOAc/petroleum ether) to yield ethyl 2-[3,4-dichloro-10-(1-tetrahydropyran-2-ylpyrazol-4-yl)-6, 7, 8, 9-tetrahydropyrido[1,2-a]indol-7-yl]acetate (110 mg, 0.219 mmol, 58% Yield). ES-MS (m/z): 475.8/477. (M+H).

### Intermediate 168

### 2-[3, 4-Dichloro-10-(1-tetrahydropyran-2-ylpyrazol-4-yl)-6, 7, 8, 9-tetrahydropyrido[1, 2-a] indol-7-yl] ethanol

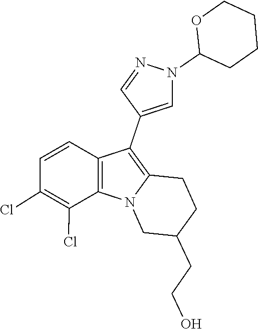 [SMILES: OCCC1CCc2c(-c3cnn(C4CCCCO4)c3)c3ccc(Cl)c(Cl)c3n2C1]

Dissolve ethyl 2-[3,4-dichloro-10-(1-tetrahydropyran-2-ylpyrazol-4-yl)-6,7,8,9-tetrahydropyrido[1,2-a]indol-7-yl]acetate (80 mg, 0.16 mmol) in DCM (3 mL) under nitrogen. Add DIBAL-H (0.80 mL, 1 mol/L in toluene) at −78° C. by syringe. Stir at RT for 3 hours. Quench with water, extract with EtOAc, dry over anhydrous sodium sulfate, filter, and concentrate. Purify by flash column chromatography (EtOAc/petroleum ether) to yield 2-[3,4-dichloro-10-(1-tetrahydropyran-2-ylpyrazol-4-yl)-6, 7, 8, 9-tetrahydropyrido[1, 2-a]indol-7-yl]ethanol (95 mg, 0.20 mmol, 92% Yield). ES-MS (m/z): 434.2/436.2 (M+H).

### EXAMPLE 73

### 2-[3, 4-Dichloro-10-(1H-pyrazol-4-yl)-6, 7, 8, 9-tetrahydropyrido[1, 2-a] indol-7-yl] ethanol

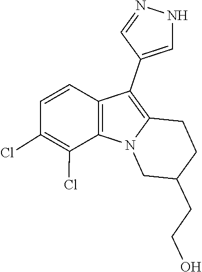 [SMILES: OCCC1CCc2c(-c3cn[nH]c3)c3ccc(Cl)c(Cl)c3n2C1]

Dissolve 2-[3,4-dichloro-10-(1-tetrahydropyran-2-ylpyrazol-4-yl)-6,7,8,9-tetrahydropyrido[1,2-a] indol-7-yl]ethanol (95 mg, 0.20 mmol) in 4N HCl/1,4-dioxane (3 mL). Stir at RT for 2 hours and concentrate. Purify by prep-HPLC to yield 2-[3,4-dichloro-10-(1H-pyrazol-4-yl)-6,7,8,9-tetrahydropyrido[1,2-a]indol-7-yl]ethanol (33 mg, 0.093 mmol, 46% Yield). ES-MS (m/z): 350.2/352.2 (M+H). ¹H NMR (400 MHz, DMSO): 12.95 (br s, 1H), 7.85 (s, 2H), 7.56 (d, J=8.5 Hz, 1H), 7.21 (d, J=8.5 Hz, 1H), 5.01 (dd, J=12.0, 5.0 Hz, 1H), 4.69-4.41 (m, 1H), 3.98 (dd, J=12.0, 10.4 Hz, 1H), 3.58 (t, J=6.4 Hz, 2H), 3.05 (ddd, J=17.0, 4.9, 3.5 Hz, 1H), 2.94 (ddd, J=17.0, 11.4, 5.6 Hz, 1H), 2.22-2.09 (m, 1H), 2.05-1.94 (m, 1H), 1.60 (q, J=6.4 Hz, 2H), 1.45 (qd, J=11.8, 5.4 Hz, 1H).

### SCHEME FOR EXAMPLE 74

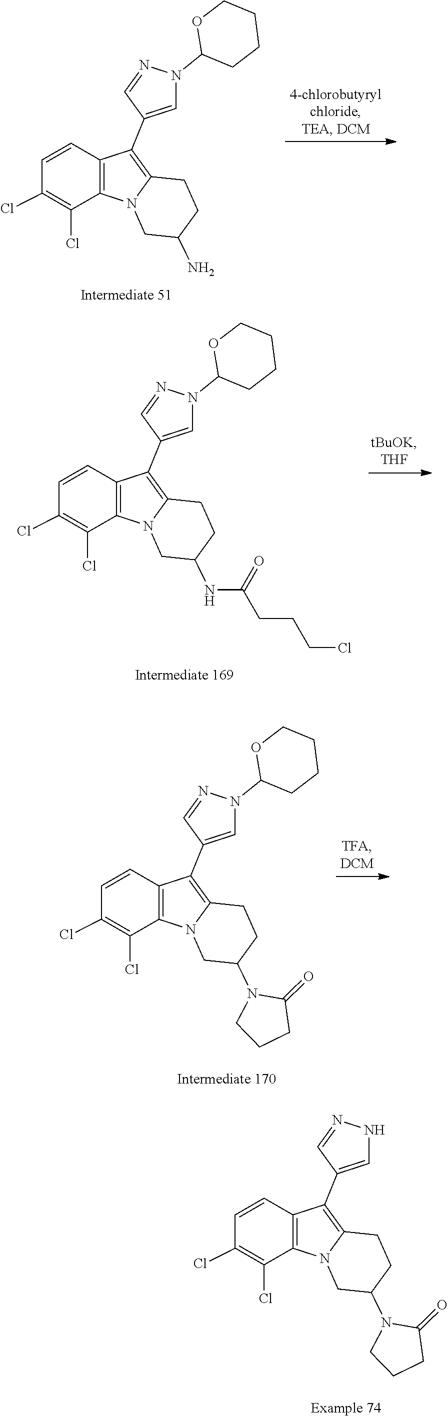 [SMILES: NC1CCc2c(-c3cnn(C4CCCCO4)c3)c3ccc(Cl)c(Cl)c3n2C1 ; O=C(CCCCl)NC1CCc2c(-c3cnn(C4CCCCO4)c3)c3ccc(Cl)c(Cl)c3n2C1 ; O=C1CCCN1C1CCc2c(-c3cnn(C4CCCCO4)c3)c3ccc(Cl)c(Cl)c3n2C1 ; O=C1CCCN1C1CCc2c(-c3cn[nH]c3)c3ccc(Cl)c(Cl)c3n2C1]

### Intermediate 169

### 4-Chloro-N-[3,4-dichloro-10-(1-tetrahydropyran-2-ylpyrazol-4-yl)-6,7,8,9-tetrahydropyrido[1,2-a]indol-7-yl]butanamide

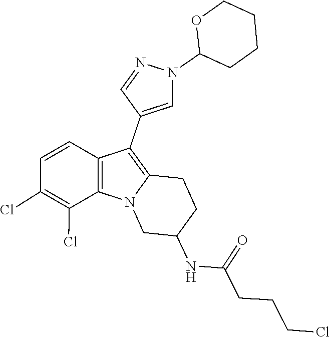 [SMILES: O=C(CCCCl)NC1CCc2c(-c3cnn(C4CCCCO4)c3)c3ccc(Cl)c(Cl)c3n2C1]

Suspend 3, 4-dichloro-10-(1-tetrahydropyran-2-ylpyrazol-4-yl)-6, 7, 8, 9-tetrahydropyrido[1,2-a]indol-7-amine (80 mg, 0.18 mmol) and TEA (0.2 mL) in DCM (3 mL). Add 4-chlorobutyryl chloride (38 mg, 0.27 mmol) at 0° C. and stir for 1 hour. Quench with saturated aqueous sodium bicarbonate and dilute with water. Extract with EtOAc, dry over anhydrous sodium sulfate, filter, and concentrate. Purify by flash column chromatography (EtOAc/petroleum ether) to yield 4-chloro-N-[3,4-dichloro-10-(1-tetrahydropyran-2-ylpyrazol-4-yl)-6,7,8,9-tetrahydropyrido[1,2-a]indol-7-yl]butanamide (65 mg, 0.11 mmol, 65% Yield). ES-MS (m/z): 509.2/511.2 (M+H).

### Intermediate 170

### 1-[3,4-Dichloro-10-(1-tetrahydropyran-2-ylpyrazol-4-yl)-6,7,8,9-tetrahydropyrido[1,2-a]indol-7-yl]pyrrolidin-2-one

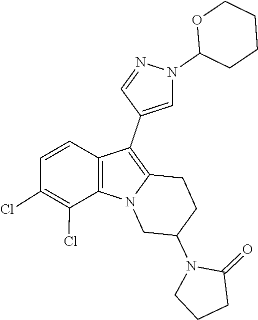 [SMILES: O=C1CCCN1C1CCc2c(-c3cnn(C4CCCCO4)c3)c3ccc(Cl)c(Cl)c3n2C1]

Suspend 4-chloro-N-[3,4-dichloro-10-(1-tetrahydropyran-2-ylpyrazol-4-yl)-6, 7, 8, 9-tetrahydropyrido[1, 2-a]indol-7-yl]butanamide (65 mg, 0.11 mmol) in THF (3 mL). Add t-BuOK (30 mg, 0.26 mmol) and stir 2 hours at RT. Quench with water and extract with EtOAc, concentrate the aqueous phase to yield 1-[3,4-dichloro-10-(1-tetrahydropyran-2-ylpyrazol-4-yl)-6, 7, 8, 9-tetrahydropyrido[1,2-a]indol-7-yl]pyrrolidin-2-one (45 mg, 0.086 mmol, 75% Yield).

### EXAMPLE 74

### 1-[3,4-Dichloro-10-(1H-pyrazol-4-yl)-6,7,8,9-tetrahydropyrido[1,2-a]indol-7-yl]pyrrolidin-2-one

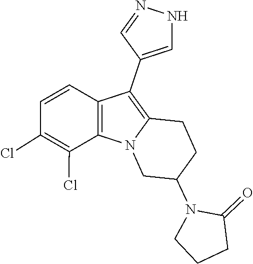 [SMILES: O=C1CCCN1C1CCc2c(-c3cn[nH]c3)c3ccc(Cl)c(Cl)c3n2C1]

Suspend 1-[3,4-dichloro-10-(1-tetrahydropyran-2-ylpyrazol-4-yl)-6, 7, 8, 9-tetrahydropyrido[1,2-a]indol-7-yl]pyrrolidin-2-one (45 mg, 0.086 mmol) in DCM (6 mL). Add TFA (2 mL) and stir for 2 hours at RT. Concentrate and purify by prep. HPLC to yield 1-[3,4-dichloro-10-(1H-pyrazol-4-yl)-6,7,8,9-tetrahydropyrido[1,2-a]indol-7-yl]pyrrolidin-2-one (2.01 mg, 0.0051 mmol, 6% Yield). ES-MS (m/z): 389.2/391.2 (M+H). ¹H NMR (400 MHz, DMSO): 13.02 (br s, 1H), 8.10-7.66 (m, 2H), 7.58 (d, J=8.5 Hz, 1H), 7.24 (d, J=8.5 Hz, 1H), 4.91 (dd, J=11.4, 5.4 Hz, 1H), 4.47-4.37 (m, 1H), 4.30 (dd, J=11.4, 10.5 Hz, 1H), 3.38-3.51 (m, 1H), 3.19-3.04 (m, 2H), 2.54-2.50 (m, 1H), 2.30 (dd, J=8.2, 7.4 Hz, 2H), 2.03-1.92 (m, 4H).

### SCHEME FOR EXAMPLE 75

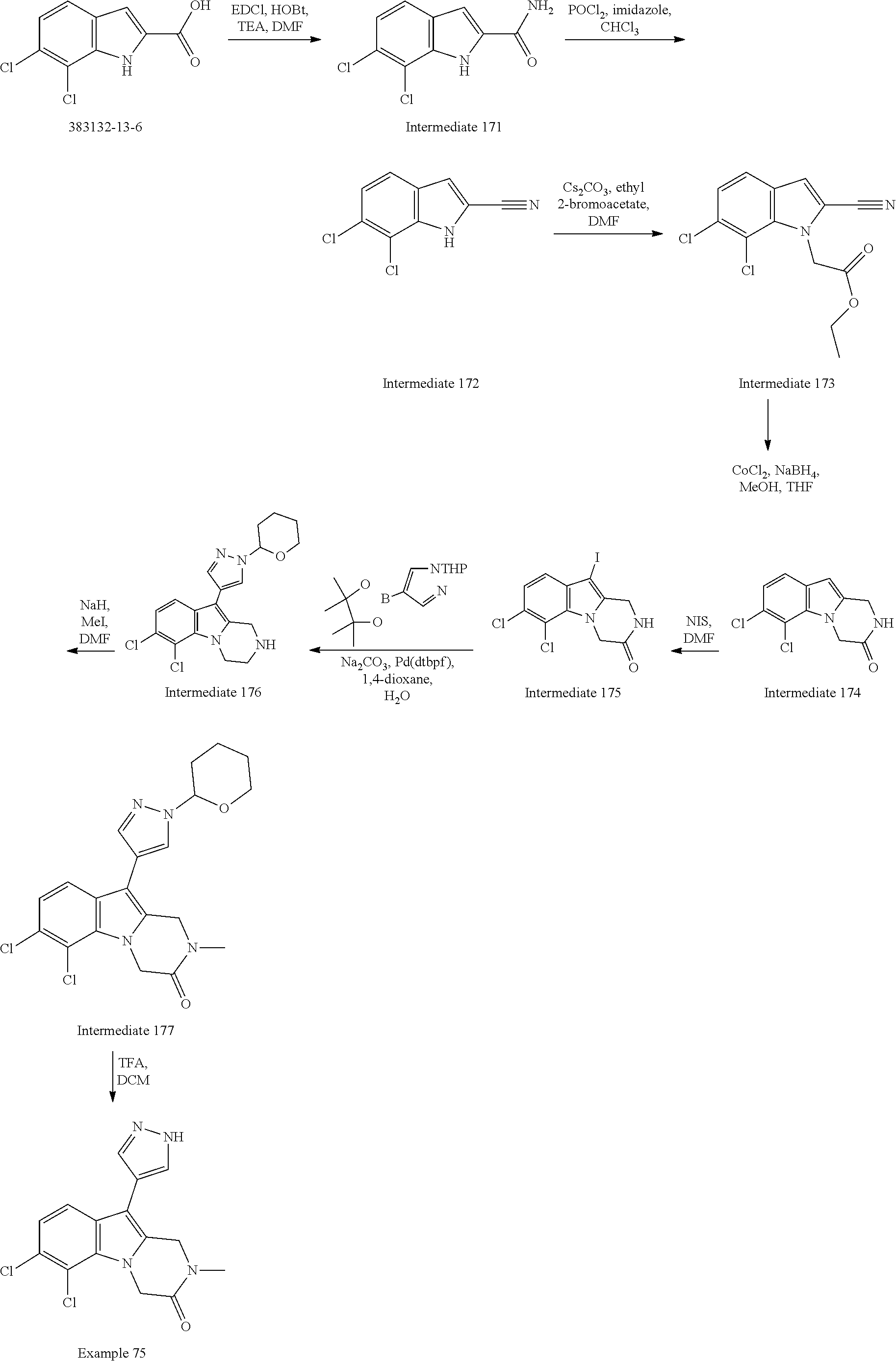

### Intermediate 171

### 6,7-Dichloro-1H-indole-2-carboxamide

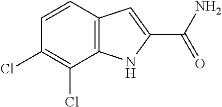 [SMILES: NC(=O)c1cc2ccc(Cl)c(Cl)c2[nH]1]

Suspend 6,7-dichloro-1H-indole-2-carboxylic acid (5.00 g, 17.4 mmol), EDCI (4.10 g, 21.0 mmol), HOBt (3.00 g, 21.1 mmol) and TEA (12 mL, 86.1 mmol) in DMF (50 mL) and stir 15 minutes at RT. Add NH₄Cl (1.90 g, 36.0 mmol) and stir overnight. Add water, extract with EtOAc, dry over anhydrous sodium sulfate, filter, and concentrate. Purify by flash column chromatography (EtOAc/petroleum ether) to yield 6,7-dichloro-1H-indole-2-carboxamide (1.84 g, 7.87 mmol, 45% Yield).

### Intermediate 172

### 6,7-Dichloro-1H-indole-2-carbonitrile

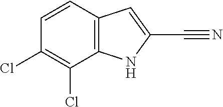 [SMILES: N#Cc1cc2ccc(Cl)c(Cl)c2[nH]1]

Suspend 3,4-dichloro-10-(1H-pyrazol-4-yl)-6,7,8,9-tetrahydropyrido[1,2-a]indole-8-carboxamide (85 mg, 0.23 mmol), POCl₃ (2.4 mL, 25 mmol) and imidazole (900 mg, 13.2 mmol) in chloroform and stir overnight at 65° C. Concentrate and quench with saturated aqueous sodium bicarbonate. Dilute water and extract with DCM. Wash with 1 N aqueous HCl. Dry over anhydrous sodium sulfate, filter and concentrate to yield 6,7-dichloro-1H-indole-2-carbonitrile (1.35 g, 6.40 mmol, 87% Yield).

### Intermediate 173

### Ethyl 2-(6,7-dichloro-2-cyano-1H-indol-1-yl)acetate

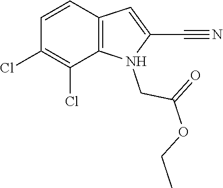 [SMILES: CCOC(=O)Cn1c(C#N)cc2ccc(Cl)c(Cl)c21]

Dissolve ethyl 6,7-dichloro-1H-indole-2-carbonitrile (1.35 g, 6.40 mmol) in DMF (15 mL). Add cesium carbonate (3.00 g, 9.20 mmol) and ethyl 2-bromoacetate (1.4 mL, 12 mmol) at RT. Stir 1.5 hours at 80° C. Add water and filter the precipitate, wash with water, then dry under vacuum to yield ethyl 2-(6,7-dichloro-2-cyano-1H-indol-1-yl)acetate (1.81 g, 6.09 mmol, 100% Yield).

### Intermediate 174

### 6,7-Dichloro-1,2-dihydropyrazino[1,2-a]indol-3(4H)-one

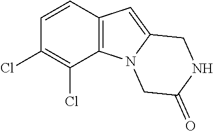 [SMILES: O=C1Cn2c(cc3ccc(Cl)c(Cl)c32)CN1]

Suspend ethyl 2-(6,7-dichloro-2-cyano-1H-indol-1-yl)acetate (1.81 g, 6.09 mmol) and CoCl₂ (1.61 g, 12.2 mmol) in methanol (20 mL) and THF (10 mL). Add NaBH₄ (1.41 g, 36.5 mmol) portion-wise at 0° C. Stir 3 hours at 0° C. Add water, extract with EtOAc, dry over anhydrous sodium sulfate, filter, and concentrate. Purify by flash column chromatography (MeOH/DCM) to yield 6,7-dichloro-1,2-dihydropyrazino[1,2-a]indol-3(4H)-one (580 mg, 2.16 mmol, 36% Yield).

### Intermediate 175

### 6,7-Dichloro-10-iodo-1,2-dihydropyrazino[1,2-a]indol-3 (4H)-one

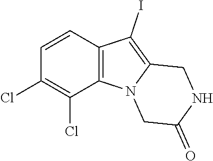 [SMILES: O=C1Cn2c(c(I)c3ccc(Cl)c(Cl)c32)CN1]

Dissolve 6,7-dichloro-1,2-dihydropyrazino[1,2-a]indol-3(4H)-one (580 mg, 2.16 mmol) in DMF (6 mL) and add NIS (600 mg, 2.59 mmol) at 0° C. Stir for 1 hour at RT. Quench with water, filter the precipitate, wash with water and MeCN to yield 6,7-dichloro-10-iodo-1,2-dihydropyrazino[1,2-a]indol-3(4H)-one (620 mg, 1.55 mmol, 95% Yield).

### Intermediate 176

### 6,7-Dichloro-10-(1-(tetrahydro-2H-pyran-2-yl)-1H-pyrazol-4-yl)-1,2-dihydropyrazin[1,2-a]indol-3 (4H)-one

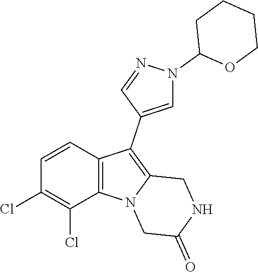 [SMILES: O=C1Cn2c(c(-c3cnn(C4CCCCO4)c3)c3ccc(Cl)c(Cl)c32)CN1]

Suspend 6,7-dichloro-10-iodo-1,2-dihydropyrazino[1,2-a]indol-3 (4H)-one (620 mg, 1.55 mmol), 1-(tetrahydro-2H-pyran-2-yl)-4-(4,4,5,5-tetramethyl-1,3,2-dioxaborolan-2-yl)-1H-pyrazole (475 mg, 1.71 mmol), Pd(dtbpf)Cl₂ (206 mg, 0.310 mmol) and Na₂CO₃ (492 mg, 4.64 mmol) in 1,4-Dioxane (6 mL) and water (3 mL). Sparge with nitrogen and heat to 90° C. for 20 min. Cool to RT, dilute with EtOAc, wash with brine, dry over anhydrous sodium sulfate, filter, and concentrate. Purify by flash column chromatography (MeOH/DCM) to yield 6,7-dichloro-10-(1-(tetrahydro-2H-pyran-2-yl)-1H-pyrazol-4-yl)-1,2-dihydropyrazin[1,2-a]indol-3(4H)-one (506 mg, 1.12 mmol, 73% Yield).

### Intermediate 177

### 6,7-Dichloro-2-methyl-10-(1-(tetrahydro-2H-pyran-2-yl)-1H-pyrazol-4-yl)-1,2-dihydropyrazino[1,2-a]indol-3(4H)-one

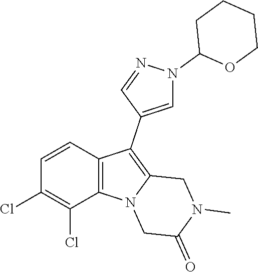 [SMILES: CN1Cc2c(-c3cnn(C4CCCCO4)c3)c3ccc(Cl)c(Cl)c3n2CC1=O]

Dissolve 6,7-dichloro-10-(1-(tetrahydro-2H-pyran-2-yl)-1H-pyrazol-4-yl)-1,2-dihydro pyrazin[1,2-a]indol-3(4H)-one (100 mg, 0.222 mmol) in DMF (2 mL). Add NaH (18 mg, 0.45 mmol, 60% dispersion in mineral oil) at 0° C. Stir 0.5 hour at 0° C. Add methyl iodide (50 mg, 0.35 mmol) at 0° C. Stir for 1 hour at RT. Add water, extract with EtOAc, wash with 4% aqueous LiCl, dry over anhydrous sodium sulfate, filter, and concentrate to yield 6,7-dichloro-2-methyl-10-(1-(tetrahydro-2H-pyran-2-yl)-1H-pyrazol-4-yl)-1,2-dihydropyrazino[1,2-a]indol-3(4H)-one (110 mg, 0.209 mmol, 95% Yield).

### EXAMPLE 75

### 6,7-Dichloro-2-methyl-10-(1H-pyrazol-4-yl)-1,2-dihydropyrazino[1,2-a]indol-3(4H)-one

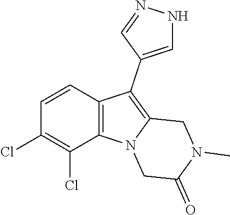 [SMILES: CN1Cc2c(-c3cn[nH]c3)c3ccc(Cl)c(Cl)c3n2CC1=O]

Suspend 6,7-dichloro-2-methyl-10-(1-(tetrahydro-2H-pyran-2-yl)-1H-pyrazol-4-yl)-1, 2-dihydropyrazino[1,2-a]indol-3(4H)-one (110 mg, 0.209 mmol) in DCM (3 mL). Add TFA (1 mL) and stir for 1 hour at RT. Concentrate and purify by prep-HPLC to yield 6,7-dichloro-2-methyl-10-(1H-pyrazol-4-yl)-1,2-dihydropyrazino[1,2-a]indol-3(4H)-one (16.9 mg, 0.050 mmol, 24% Yield). ES-MS (m/z): 335.2/337.2 (M+H). ¹H NMR (400 MHz, DMSO): 13.11 (br s, 1H), 8.07 (br s, 1H), 7.80 (br s, 1H), 7.68 (d, J=8.5 Hz, 1H), 7.30 (d, J=8.5 Hz, 1H), 5.27 (s, 2H), 4.81 (s, 2H), 3.06 (s, 3H).

### SCHEME FOR EXAMPLE 76

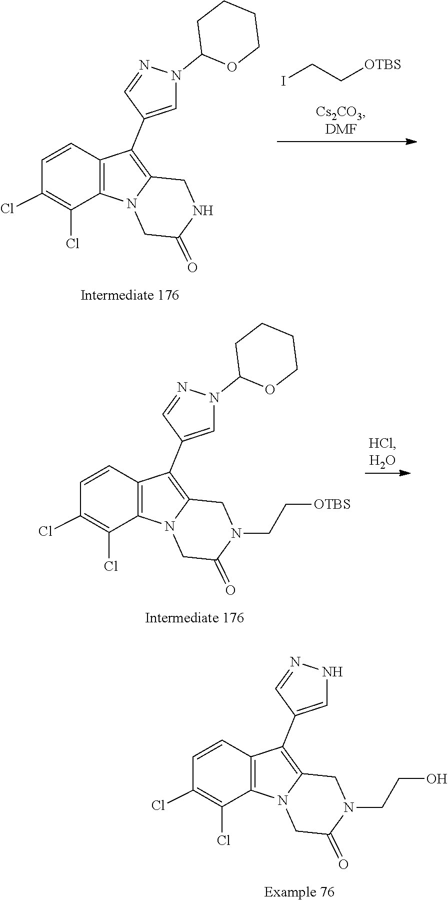

### Intermediate 178

### 2-(2-((tert-Butyldimethylsilyl)oxy)ethyl)-6,7-dichloro-10-(1-(tetrahydro-2H-pyran-2-yl)-1H-pyrazol-4-yl)-1,2-dihydropyrazino[1,2-a]indol-3(4H)-one

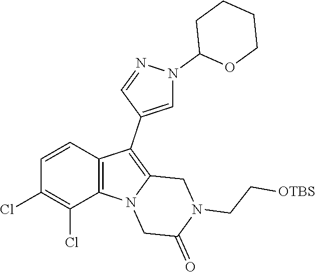 [SMILES: CC(C)(C)[Si](C)(C)OCCN1Cc2c(-c3cnn(C4CCCCO4)c3)c3ccc(Cl)c(Cl)c3n2CC1=O]

Dissolve 6,7-dichloro-10-(1-(tetrahydro-2H-pyran-2-yl)-1H-pyrazol-4-yl)-1,2-dihydro pyrazin[1,2-a]indol-3(4H)-one (100 mg, 0.222 mmol) in DMF (4 mL) and add cesium carbonate (50 mg, 0.15 mmol) and tert-butyl(2-iodoethoxy)dimethylsilane (140 mg, 0.465 mmol) at RT. Stir for 2 hours at 60° C. under nitrogen. Quench with water, extract with EtOAc, wash with 4% aqueous LiCl, dry over anhydrous sodium sulfate, filter and concentrate to yield 2-(2-((tert-butyldimethylsilyl)oxy)ethyl)-6,7-dichloro-10-(1-(tetrahydro-2H-pyran-2-yl)-1H-pyrazol-4-yl)-1,2-dihydropyrazino[1,2-a]indol-3 (4H)-one (150 mg, 0.212 mmol, 96% Yield).

### EXAMPLE 76

### 6,7-Dichloro-2-(2-hydroxyethyl)-10-(1H-pyrazol-4-yl)-1,2-dihydropyrazino[1,2-a]indol-3(4H)-one

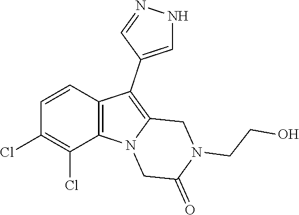 [SMILES: O=C1Cn2c(c(-c3cn[nH]c3)c3ccc(Cl)c(Cl)c32)CN1CCO]

Suspend 2-(2-((tert-butyldimethylsilyl)oxy)ethyl)-6,7-dichloro-10-(1-(tetrahydro-2H-pyran-2-yl)-1H-pyrazol-4-yl)-1,2-dihydropyrazino[1,2-a]indol-3(4H)-one (150 mg, 0.212 mmol) in 6 N aqueous HCl (4 mL) and stir for 1 hour at RT. Concentrate and purify by prep-HPLC to yield 6,7-dichloro-2-(2-hydroxyethyl)-10-(1H-pyrazol-4-yl)-1,2-dihydropyrazino[1,2-a]indol-3(4H)-one. (25 mg, 0.068 mmol, 32% Yield). ES-MS (m/z): 365.0/367.0 (M+H). ¹H NMR (400 MHz, DMSO): 7.93 (s, 2H), 7.68 (d, J=8.5 Hz, 1H), 7.30 (d, J=8.5 Hz, 1H), 5.29 (s, 2H), 4.90 (s, 2H), 3.64-3.56 (m, 4H).

### SCHEME FOR EXAMPLE 77

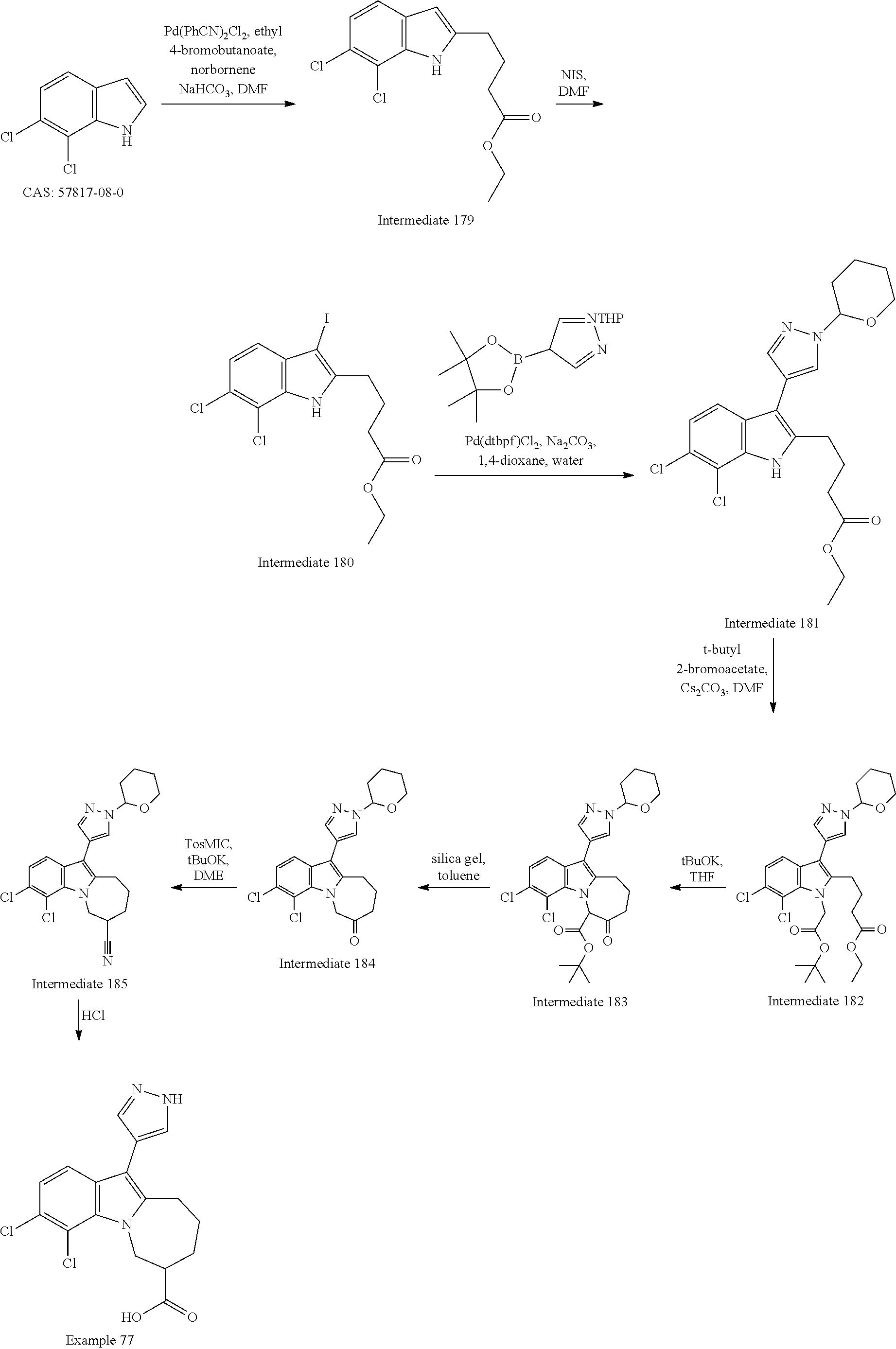

### Intermediate 179

### Ethyl 4-(6,7-dichloro-1H-indol-2-yl)butanoate

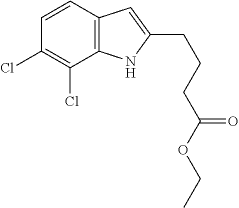 [SMILES: CCOC(=O)CCCc1cc2ccc(Cl)c(Cl)c2[nH]1]

Suspend 6,7-dichloro-1H-indole (5.00 g, 25.5 mmol), ethyl 4-bromobutanoate (6.30 g, 31.0 mmol), Pd(PhCN)₂Cl₂ (2.00 g, 5.16 mmol), norborn-2-ene (10.0 g, 105 mmol) and NaHCO₃ (8.70 g, 100 mmol) in DMF (50 mL) and water (0.73 mL). Sparge with nitrogen and heat to 70° C. overnight. Cool to RT, add water, extract with EtOAc, dry over anhydrous sodium sulfate, filter, and concentrate. Purify by flash column chromatography (EtOAc/petroleum) to yield ethyl 4-(6,7-dichloro-1H-indol-2-yl)butanoate (7.04 g, 23.0 mmol, 90% Yield).

### Intermediate 180

### Ethyl 4-(6,7-dichloro-3-iodo-1H-indol-2-yl)butanoate

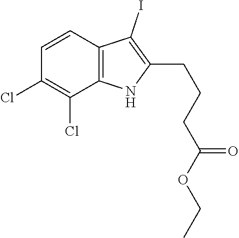 [SMILES: CCOC(=O)CCCc1[nH]c2c(Cl)c(Cl)ccc2c1I]

Dissolve ethyl 4-(6,7-dichloro-1H-indol-2-yl)butanoate (7.04 g, 23.0 mmol) in DMF (100 mL) and add NIS (6.40 g, 28.0 mmol). Stir overnight at RT. Quench with water and stir vigorously for 15 minutes. Add water, extract with EtOAc, dry over anhydrous sodium sulfate, filter and concentrate. Purify by flash column chromatography (EtOAc/petroleum) to yield ethyl 4-(6,7-dichloro-3-iodo-1H-indol-2-yl)butanoate (7.07 g, 16.3 mmol, 71% Yield).

### Intermediate 181

### Ethyl 4-(6,7-dichloro-3-(1-(tetrahydro-2H-pyran-2-yl)-1H-pyrazol-4-yl)-1H-indol-2-yl)butanoate

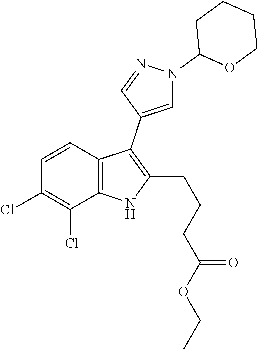 [SMILES: CCOC(=O)CCCc1[nH]c2c(Cl)c(Cl)ccc2c1-c1cnn(C2CCCCO2)c1]

Suspend ethyl 4-(6,7-dichloro-3-iodo-1H-indol-2-yl)butanoate (7.07 g, 16.3 mmol), 1-(tetrahydro-2H-pyran-2-yl)-4-(4,4,5,5-tetramethyl-1,3,2-dioxaborolan-2-yl)-1H-pyrazole (10.0 g, 34.2 mmol), Pd(dppf)Cl₂ (1.90 g, 2.50 mmol) and Na₂CO₃ (5.30 g, 50.0 mmol) in 1,4-dioxane (40 mL) and water (10 mL). Sparge with nitrogen and heat to 90° C. overnight. Cool to RT, add water, extract with EtOAc, dry over anhydrous sodium sulfate, filter, and concentrate. Purify by flash column chromatography (EtOAc/petroleum) to yield ethyl 4-(6,7-dichloro-3-(1-(tetrahydro-2H-pyran-2-yl)-1H-pyrazol-4-yl)-1H-indol-2-yl)butanoate (3.79 g, 7.74 mmol, 48% Yield) as a yellow solid. ES-MS (m/z): 450.0/452.0 (M+H).

### Intermediate 182

### Ethyl 4-(1-(2-(tert-butoxy)-2-oxoethyl)-6,7-dichloro-3-(1-(tetrahydro-2H-pyran-2-yl)-1H-pyrazol-4-yl)-1H-indol-2-yl)butanoate

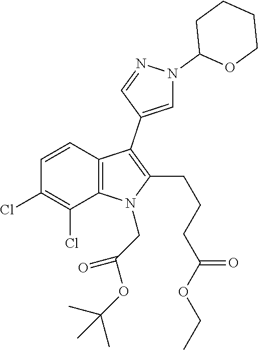 [SMILES: CCOC(=O)CCCc1c(-c2cnn(C3CCCCO3)c2)c2ccc(Cl)c(Cl)c2n1CC(=O)OC(C)(C)C]

Suspend ethyl 4-(6,7-dichloro-3-(1-(tetrahydro-2H-pyran-2-yl)-1H-pyrazol-4-yl)-1H-indol-2-yl) butanoate (3.79 g, 7.74 mmol) and Cs₂CO₃ (8.80 g, 27.0 mmol) in DMF (30 mL). Add tert-butyl 2-bromoacetate (3.00 g, 15.2 mmol) at 0° C. Stir overnight at RT. Add water, extract with EtOAc, dry over anhydrous sodium sulfate, filter, and concentrate. Purify by flash column chromatography (EtOAc/petroleum) to yield ethyl 4-(1-(2-(tert-butoxy)-2-oxoethyl)-6,7-dichloro-3-(1-(tetrahydro-2H-pyran-2-yl)-1H-pyrazol-4-yl)-1H-indol-2-yl)butanoate (2.81 g, 4.83 mmol, 62% Yield). ES-MS (m/z): 564.6/566.2 (M+H).

### Intermediate 183

### tert-Butyl 3,4-dichloro-7-oxo-11-(1-(tetrahydro-2H-pyran-2-yl)-1H-pyrazol-4-yl)-7,8,9,10-tetrahydro-6H-azepino[1,2-a]indole-6-carboxylate

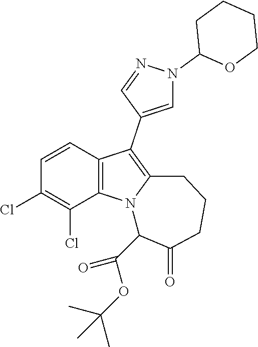 [SMILES: CC(C)(C)OC(=O)C1C(=O)CCCc2c(-c3cnn(C4CCCCO4)c3)c3ccc(Cl)c(Cl)c3n21]

Dissolve ethyl 4-(1-(2-(tert-butoxy)-2-oxoethyl)-6,7-dichloro-3-(1-(tetrahydro-2H-pyran-2-yl)-1H-pyrazol-4-yl)-1H-indol-2-yl)butanoate (2.81 g, 4.83 mmol) in THF (10 mL) and add t-BuOK (19 mL, 19 mmol, 1 mol/L in THF). Stir overnight at RT. Add water, extract with EtOAc, dry over anhydrous sodium sulfate, filter, and concentrate to yield tert-butyl 3,4-dichloro-7-oxo-11-(1-(tetrahydro-2H-pyran-2-yl)-1H-pyrazol-4-yl)-7,8,9,10-tetrahydro-6H-azepino[1,2-a]indole-6-carboxylate (2.27 g, 2.45 mmol, 51% Yield). ES-MS (m/z): 518.1/520.1 (M+H).

### Intermediate 184

### 3,4-Dichloro-11-(1-(tetrahydro-2H-pyran-2-yl)-1H-pyrazol-4-yl)-9,10-dihydro-6H-azepino[1,2-a]indol-7(8H)-one

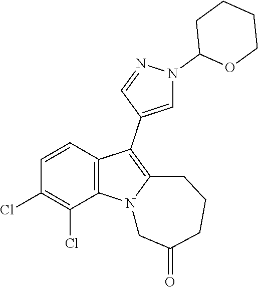 [SMILES: O=C1CCCc2c(-c3cnn(C4CCCCO4)c3)c3ccc(Cl)c(Cl)c3n2C1]

Dissolve tert-butyl 3,4-dichloro-7-oxo-11-(1-(tetrahydro-2H-pyran-2-yl)-1H-pyrazol-4-yl)-7, 8, 9, 10-tetrahydro-6H-azepino[1,2-a]indole-6-carboxylate (2.21 g, 2.39 mmol) in toluene (30 mL) and add silica gel (2.00 g). Heat to 120° C. under nitrogen overnight. Cool to RT and concentrate. Purify by flash column chromatography (EtOAc/petroleum) to yield 3,4-dichloro-11-(1-(tetrahydro-2H-pyran-2-yl)-1H-pyrazol-4-yl)-9,10-dihydro-6H-azepino [1,2-a]indol-7(8H)-one (770 mg, 1.75 mmol, 73% Yield). ES-MS (m/z): 418.0/420.0 (M+H).

### Intermediate 185

### 3,4-Dichloro-11-(1-(tetrahydro-2H-pyran-2-yl)-1H-pyrazol-4-yl)-7,8,9,10-tetrahydro-6H-azepino[1,2-a]indole-7-carbonitrile

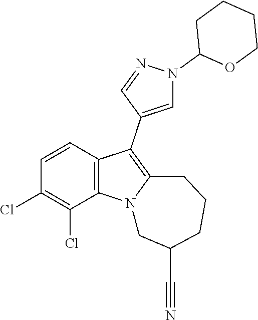 [SMILES: N#CC1CCCc2c(-c3cnn(C4CCCCO4)c3)c3ccc(Cl)c(Cl)c3n2C1]

Dissolve 3,4-dichloro-11-(1-(tetrahydro-2H-pyran-2-yl)-1H-pyrazol-4-yl)-9,10-dihydro-6H-azepino[1,2-a]indol-7(8H)-one (550 mg, 1.25 mmol) and TosMIC (330 mg, 1.64 mmol) in 1,2-dimethoxyethane (5 mL). Add t-BuOK (425 mg, 3.75 mmol) at 0° C. Stir overnight at RT. Add water, extract with EtOAc, dry over anhydrous sodium sulfate, filter, and concentrate. Purify by flash column chromatography (EtOAc/petroleum ether) to yield 3,4-dichloro-11-(1-(tetrahydro-2H-pyran-2-yl)-1H-pyrazol-4-yl)-7, 8,9,10-tetrahydro-6H-azepino[1,2-a]indole-7-carbonitrile (280 mg, 0.574 mmol, 46% Yield). ES-MS (m/z): 429.0/431.0 (M+H).

### EXAMPLE 77

### 3,4-Dichloro-11-(1H-pyrazol-4-yl)-7,8,9,10-tetrahydro-6H-azepino[1,2-a]indole-7-carboxylic acid

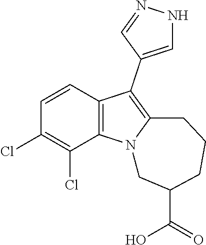 [SMILES: O=C(O)C1CCCc2c(-c3cn[nH]c3)c3ccc(Cl)c(Cl)c3n2C1]

Dissolve 3,4-dichloro-11-(1-(tetrahydro-2H-pyran-2-yl)-1H-pyrazol-4-yl)-7, 8, 9, 10-tetrahydro-6H-azepino[1,2-a]indole-7-carbonitrile (100 mg, 0.205 mmol) in conc. HCl (4 mL). Stir overnight at 100° C. Concentrate and purify by prep-HPLC to yield 3,4-dichloro-11-(1H-pyrazol-4-yl)-7,8,9,10-tetrahydro-6H-azepino[1,2-a]indole-7-carboxylic acid (32 mg, 0.43 mmol, 43% Yield). ES-MS (m/z): 364.0/366.0 (M+H). ¹H NMR (400 MHz, DMSO): 7.77 (s, 2H), 7.44 (d, J=8.4 Hz, 1H), 7.22 (d, J=8.4 Hz, 1H), 5.50 (d, J=15.0 Hz, 1H), 4.58 (dd, J=15.0, 8.6 Hz, 1H), 3.07 (dd, J=15.0, 7.5 Hz, 1H), 2.85 (dd, J=15.0, 10.5 Hz, 1H), 2.70-2.60 (m, 1H), 2.14-1.94 (m, 2H), 1.93-1.81 (m, 1H), 1.58-1.44 (m, 1H).

### SCHEME FOR EXAMPLE 78

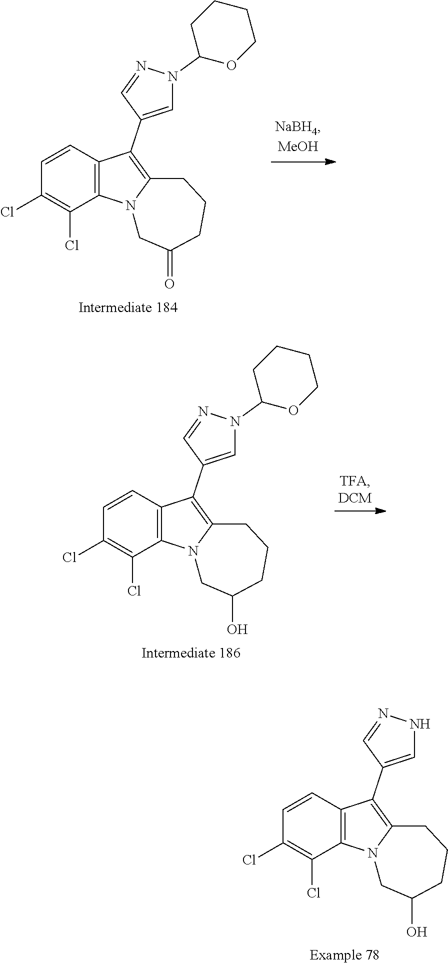 [SMILES: O=C1CCCc2c(-c3cnn(C4CCCCO4)c3)c3ccc(Cl)c(Cl)c3n2C1 ; OC1CCCc2c(-c3cnn(C4CCCCO4)c3)c3ccc(Cl)c(Cl)c3n2C1 ; OC1CCCc2c(-c3cn[nH]c3)c3ccc(Cl)c(Cl)c3n2C1]

### Intermediate 186

### 3,4-Dichloro-11-(1-(tetrahydro-2H-pyran-2-yl)-1H-pyrazol-4-yl)-7,8,9,10-tetrahydro-6H-azepino[1,2-a]indol-7-ol

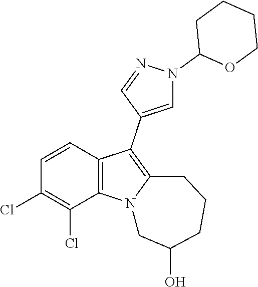 [SMILES: OC1CCCc2c(-c3cnn(C4CCCCO4)c3)c3ccc(Cl)c(Cl)c3n2C1]

Dissolve 3, 4-dichloro-11-(1-(tetrahydro-2H-pyran-2-yl)-1H-pyrazol-4-yl)-9, 10-dihydro-6H-azepino[1,2-a]indol-7(8H)-one (87 mg, 0.20 mmol) in MeOH (2 mL) and add NaBH₄ (230 mg, 5.84 mmol) at 0° C. Stir for 2 hours at RT. Quench with saturated NH₄Cl solution. Stir 30 min. Add water, extract with EtOAc, dry over anhydrous sodium sulfate, filter, and concentrate to yield 3,4-dichloro-11-(1-(tetrahydro-2H-pyran-2-yl)-1H-pyrazol-4-yl)-7, 8, 9, 10-tetrahydro-6H-azepino[1,2-a]indol-7-ol (60 mg, 0.13 mmol, 66% Yield). ES-MS (m/z): 420.0/422.0 (M+H).

### EXAMPLE 78

### 3,4-Dichloro-11-(1H-pyrazol-4-yl)-7,8,9,10-tetrahydro-6H-azepino[1,2-a]indol-7-ol

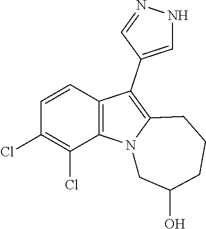 [SMILES: OC1CCCc2c(-c3cn[nH]c3)c3ccc(Cl)c(Cl)c3n2C1]

Suspend 3,4-dichloro-11-(1-(tetrahydro-2H-pyran-2-yl)-1H-pyrazol-4-yl)-7, 8, 9, 10-tetrahydro-6H-azepino[1,2-a]indol-7-ol (60 mg, 0.13 mmol) in DCM (2 mL). Add TFA (2 mL) and stir for 3 hours at RT. Concentrate and dilute with EtOAc and water, adjust to pH 8 with saturated aqueous NaHCO₃, extract with EtOAc, wash with saturated aqueous sodium bicarbonate (3×), brine, dry over anhydrous sodium sulfate, filter, and concentrate. Purify by prep-HPLC to yield 3,4-dichloro-11-(1H-pyrazol-4-yl)-7,8,9,10-tetrahydro-6H-azepino[1,2-a]indol-7-ol (32 mg, 0.0943 mmol, 72% Yield). ES-MS (m/z): 336.2/338.2 (M+H). ¹H NMR (400 MHz, DMSO): 7.74 (s, 2H), 7.43 (d, J=8.4 Hz, 1H), 7.20 (d, J=8.4 Hz, 1H), 5.10 (d, J=14.5 Hz, 1H), 4.51 (dd, J=14.5, 8.6 Hz, 1H), 3.77-3.70 (m, 1H), 3.00 (dd, J=15.0, 7.6 Hz, 1H), 2.85 (dd, J=15.0, 10.0 Hz, 1H), 2.01-1.81 (m, 2H), 1.73-1.61 (m, 1H), 1.54-1.41 (m, 1H).

### SCHEME FOR EXAMPLE 79

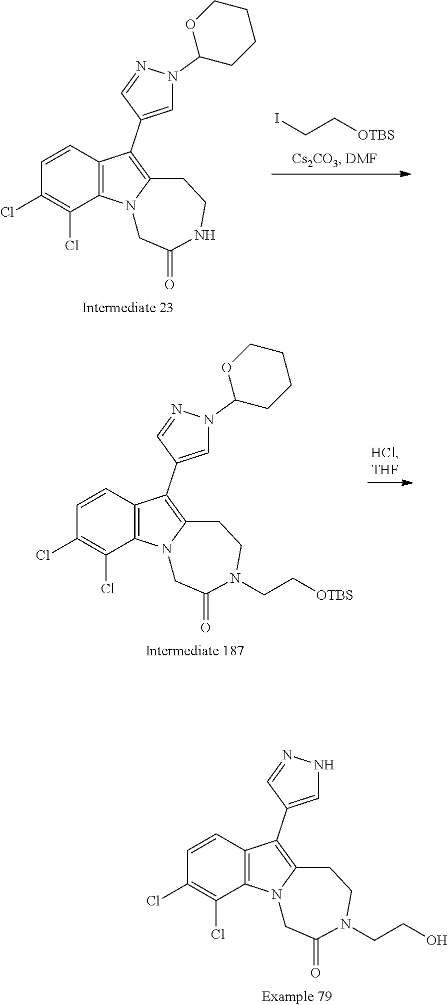 [SMILES: O=C1Cn2c(c(-c3cnn(C4CCCCO4)c3)c3ccc(Cl)c(Cl)c32)CCN1 ; CC(C)(C)[Si](C)(C)OCCI ; CC(C)(C)[Si](C)(C)OCCN1CCc2c(-c3cnn(C4CCCCO4)c3)c3ccc(Cl)c(Cl)c3n2CC1=O ; O=C1Cn2c(c(-c3cn[nH]c3)c3ccc(Cl)c(Cl)c32)CCN1CCO]

### Intermediate 187

### 3-(2-((tert-Butyldimethylsilyl)oxy)ethyl)-7,8-dichloro-11-(1-(tetrahydro-2H-pyran-2-yl)-1H-pyrazol-4-yl)-2,3-dihydro-1H-[1,4]diazepino[1,7-a]indol-4(5H)-one

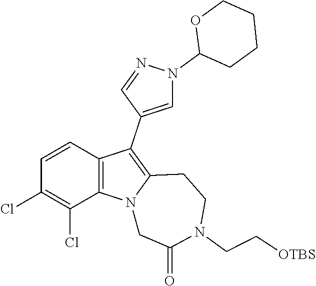 [SMILES: CC(C)(C)[Si](C)(C)OCCN1CCc2c(-c3cnn(C4CCCCO4)c3)c3ccc(Cl)c(Cl)c3n2CC1=O]

Suspend 7,8-dichloro-11-(1-(tetrahydro-2H-pyran-2-yl)-1H-pyrazol-4-yl)-2, 3-dihydro-1H-[1,4]diazepino[1,7-a]indol-4(5H)-one (200 mg, 0.467 mmol), Cs₂CO₃ (300 mg, 0.920 mmol) in DMF (3 mL) and add tert-butyl(2-iodoethoxy)dimethylsilane (280 mg, 0.958 mmol). Stir for 32 hours at 80° C. Add water, extract with EtOAc, dry over anhydrous sodium sulfate, filter, and concentrate. Purify by flash column chromatography (MeOH/DCM) to yield 3-(2-((tert-butyldimethylsilyl)oxy)ethyl)-7,8-dichloro-11-(1-(tetrahydro-2H-pyran-2-yl)-1H-pyrazol-4-yl)-2,3-dihydro-1H-[1,4]diazepino[1,7-a]indol-4(5H)-one (137 mg, 0.168 mmol, 36% Yield). ES-MS (m/z): 577.2/579.2 (M+H).

### EXAMPLE 79

### 7,8-Dichloro-3-(2-hydroxyethyl)-11-(1H-pyrazol-4-yl)-2,3-dihydro-1H-[1,4]diazepino[1,7-a]indol-4(5H)-one

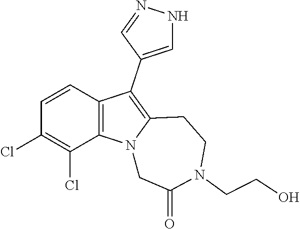 [SMILES: O=C1Cn2c(c(-c3cn[nH]c3)c3ccc(Cl)c(Cl)c32)CCN1CCO]

Suspend 3-(2-((tert-butyldimethylsilyl)oxy)ethyl)-7, 8-dichloro-11-(1-(tetrahydro-2H-pyran-2-yl)-1H-pyrazol-4-yl)-2,3-dihydro-1H-[1,4]diazepino[1,7-a]indol-4(5H)-one (137 mg, 0.168 mmol) in THF (2 mL). Add 6 N aqueous hydrogen chloride (1 mL) and stir for 2 hours at RT. Concentrate and dilute with EtOAc and water, adjust to pH 8 with saturated aqueous NaHCO₃, extract with EtOAc, wash with saturated aqueous sodium bicarbonate (3×), brine, dry over anhydrous sodium sulfate, filter, and concentrate. Purify by prep-HPLC to yield 7,8-dichloro-3-(2-hydroxyethyl)-11-(1H-pyrazol-4-yl)-2,3-dihydro-1H-[1,4]diazepino[1,7-a]indol-4(5H)-one (11 mg, 0.030 mmol, 18% Yield). ES-MS (m/z): 379.2/381.2 (M+H). ¹H NMR (400 MHz, DMSO): 13.00 (br s, 1H), 7.86 (br s, 2H), 7.54 (d, J=8.5 Hz, 1H), 7.28 (d, J=8.5 Hz, 1H), 5.60 (s, 2H), 4.74 (br s, 1H), 3.82 (t, J=6.1 Hz, 2H), 3.46-3.43 (m, 2H), 3.40-3.36 (m, 4H).

### SCHEME FOR EXAMPLE 80

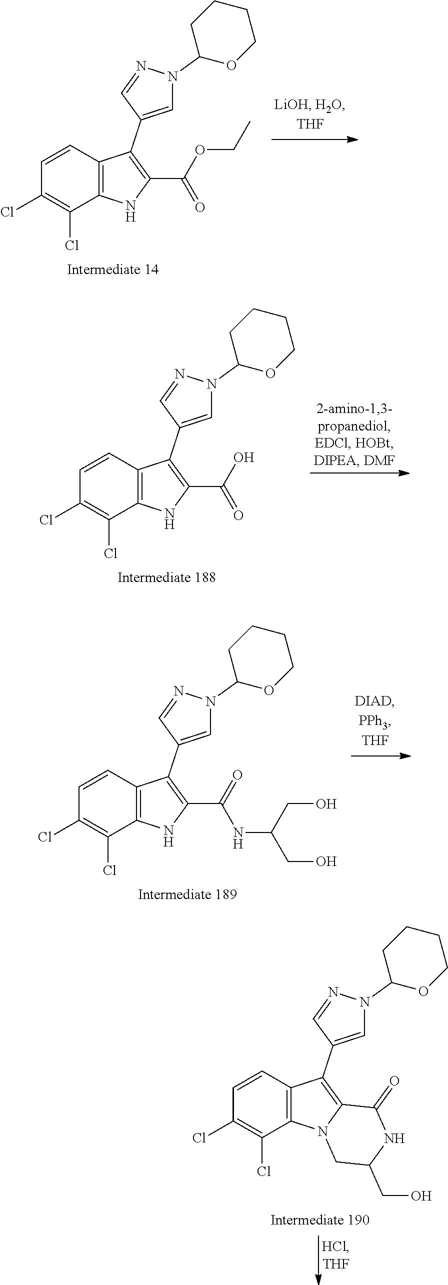 [SMILES: CCOC(=O)c1[nH]c2c(Cl)c(Cl)ccc2c1-c1cnn(C2CCCCO2)c1 ; O=C(O)c1[nH]c2c(Cl)c(Cl)ccc2c1-c1cnn(C2CCCCO2)c1 ; O=C(NC(CO)CO)c1[nH]c2c(Cl)c(Cl)ccc2c1-c1cnn(C2CCCCO2)c1 ; O=C1NC(CO)Cn2c1c(-c1cnn(C3CCCCO3)c1)c1ccc(Cl)c(Cl)c12]

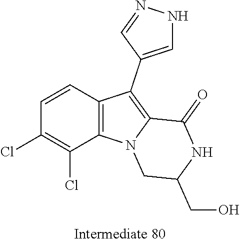 [SMILES: O=C1NC(CO)Cn2c1c(-c1cn[nH]c1)c1ccc(Cl)c(Cl)c12]

### Intermediate 188

### 6,7-Dichloro-3-(1-tetrahydropyran-2-ylpyrazol-4-yl)-1H-indole-2-carboxylic acid

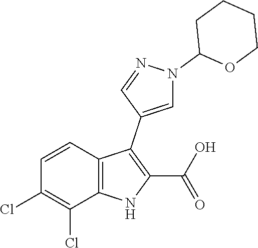 [SMILES: O=C(O)c1[nH]c2c(Cl)c(Cl)ccc2c1-c1cnn(C2CCCCO2)c1]

Dissolve ethyl 6, 7-dichloro-3-(1-tetrahydropyran-2-ylpyrazol-4-yl)-1H-indole-2-carboxylate (1.00 g, 1.96 mmol, 80% purity) in THF (12 mL) and H₂O (3 mL). Add LiOH·H₂O (700 mg, 28.6 mmol) and stir at 60° C. overnight. Acidify with 1N aqueous HCl to pH 3, extract with DCM, dry over anhydrous sodium sulfate, filter and concentrate to yield 6,7-dichloro-3-(1-tetrahydropyran-2-ylpyrazol-4-yl)-1H-indole-2-carboxylic acid (800 mg, 1.89 mmol, 97% Yield). ES-MS (m/z): 380.0/382.0 (M+H).

### Intermediate 189

### 6, 7-Dichloro-N-[2-hydroxy-1-(hydroxymethyl) ethyl]-3-(1-tetrahydropyran-2-ylpyrazol-4-yl)-1H-indole-2-carboxamide

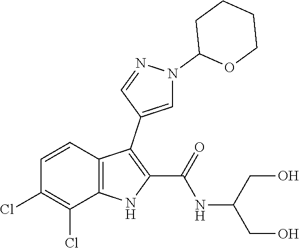 [SMILES: O=C(NC(CO)CO)c1[nH]c2c(Cl)c(Cl)ccc2c1-c1cnn(C2CCCCO2)c1]

Dissolve 6, 7-dichloro-3-(1-tetrahydropyran-2-ylpyrazol-4-yl)-1H-indole-2-carboxylic acid (800 mg, 2.04 mmol), 2-amino-1,3-propanediol (244 mg, 2.65 mmol) and DIPEA (1.80 mL, 10.0 mmol) in DMF (10 mL). Add EDCI (520 mg, 2.66 mmol) and HOBt (380 mg, 2.67 mmol). Stir at RT overnight. Concentrate and purify by flash column chromatography (MeOH/DCM) and then by prep-HPLC to yield 6, 7-dichloro-N-[2-hydroxy-1-(hydroxymethyl)ethyl]-3-(1-tetrahydropyran-2-ylpyrazol-4-yl)-1H-indole-2-carboxamide (650 mg, 1.43 mmol, 70% Yield). ES-MS (m/z): 453.0/454.9 (M+H).

### Intermediate 190

### 6, 7-Dichloro-3-(hydroxymethyl)-10-(1-tetrahydropyran-2-ylpyrazol-4-yl)-3, 4-dihydro-2H-pyrazino[1, 2-a] indol-1-one

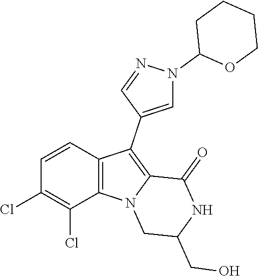 [SMILES: O=C1NC(CO)Cn2c1c(-c1cnn(C3CCCCO3)c1)c1ccc(Cl)c(Cl)c12]

Dissolve 6, 7-dichloro-N-[2-hydroxy-1-(hydroxymethyl)ethyl]-3-(1-tetrahydropyran-2-ylpyrazol-4-yl)-1H-indole-2-carboxamide (400 mg, 0.882 mmol) and triphenylphosphine (830 mg, 3.10 mmol) in THF (20 mL). Add diisopropyl azodicarboxylate (0.65 mL, 3.3 mmol) dropwise by syringe at 0° C. under nitrogen. Stir at RT for 3 days. Concentrate and purify by flash column chromatography (MeOH/DCM) to yield 6, 7-dichloro-3-(hydroxymethyl)-10-(1-tetrahydropyran-2-ylpyrazol-4-yl)-3, 4-dihydro-2H-pyrazino[1, 2-a] indol-1-one (130 mg, 0.173 mmol, 20% Yield). ES-MS (m/z): 435.0/437.0 (M+H).

### EXAMPLE 80

### 6, 7-Dichloro-3-(hydroxymethyl)-10-(1H-pyrazol-4-yl)-3, 4-dihydro-2H-pyrazino[1, 2-a] indol-1-one

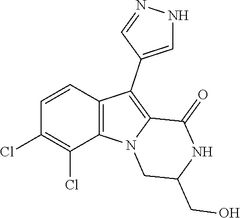 [SMILES: O=C1NC(CO)Cn2c1c(-c1cn[nH]c1)c1ccc(Cl)c(Cl)c12]

Dissolve 6, 7-dichloro-3-(hydroxymethyl)-10-(1-tetrahydropyran-2-ylpyrazol-4-yl)-3, 4-dihydro-2H-pyrazino[1, 2-a] indol-1-one (130 mg, 0.173 mmol) in THF (2 mL). Add 6 N aqueous HCl (2 mL), Stir at RT for 2 hours. Purify by prep-HPLC to yield 6, 7-dichloro-3-(hydroxymethyl)-10-(1H-pyrazol-4-yl)-3,4-dihydro-2H-pyrazino[1,2-a]indol-1-one (product, 17 mg, 0.047 mmol, 27% Yield). ES-MS (m/z): 351.0/353.0 (M+H). ¹H NMR (400 MHz, DMSO): 12.94 (br s, 1H), 8.24 (d, J=3.1 Hz, 1H), 8.15 (br s, 1H), 7.89 (br s, 1H), 7.74 (d, J=8.6 Hz, 1H), 7.32 (d, J=8.6 Hz, 1H), 5.14 (t, J=5.2 Hz, 1H), 4.93 (dd, J=12.7, 5.8 Hz, 1H), 4.81 (dd, J=12.7, 4.2 Hz, 1H), 3.80-3.71 (m, 1H), 3.58-3.50 (m, 1H), 3.36-3.27 (m, 1H).

### SCHEME FOR EXAMPLE 81

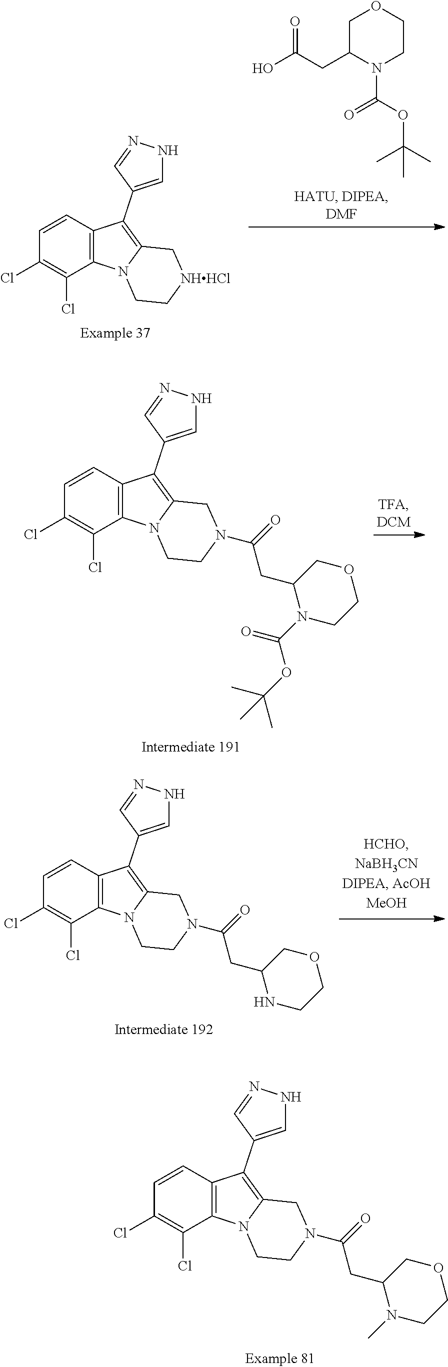

### Intermediate 191

### tert-Butyl 3-[2-[6,7-dichloro-10-(1H-pyrazol-4-yl)-3,4-dihydro-1H-pyrazino[1,2-a]indol-2-yl]-2-oxo-ethyl]morpholine-4-carboxylate

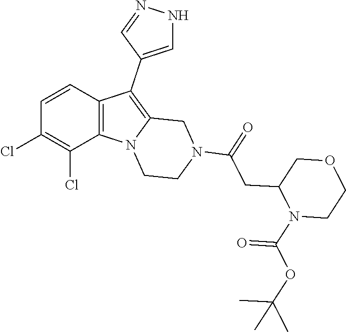 [SMILES: CC(C)(C)OC(=O)N1CCOCC1CC(=O)N1CCn2c(c(-c3cn[nH]c3)c3ccc(Cl)c(Cl)c32)C1]

Suspend 6, 7-dichloro-10-(1H-pyrazol-4-yl)-1,2,3,4-tetrahydropyrazino[1,2-a]indole (170 mg, 0.470 mmol, HCl salt) in DMF (8 mL). Add 2-(4-tert-butoxycarbonylmorpholin-3-yl)acetic acid (127 mg, 0.518 mmol), DIPEA (183 mg, 1.42 mmol) and HATU (220 mg, 0.567 mmol) at RT. Stir for 2 hours at RT. Dilute with H₂O, extract with EtOAc, wash with brine, dry over anhydrous sodium sulfate, filter, and concentrate. Add MeOH (4 mL) and K₂CO₃ (195 mg, 1.41 mmol) to the residue and stir overnight at RT. Dilute with H₂O, extract with EtOAc, wash with brine, dry over anhydrous sodium sulfate, filter and concentrate to yield tert-butyl 3-[2-[6,7-dichloro-10-(1H-pyrazol-4-yl)-3,4-dihydro-1H-pyrazino[1,2-a]indol-2-yl]-2-oxo-ethyl]morpholine-4-carboxylate (290 mg, 0.516 mmol, crude).

### Intermediate 192

### 1-[6,7-Dichloro-10-(1H-pyrazol-4-yl)-3,4-dihydro-1H-pyrazino[1,2-a]indol-2-yl]-2-morpholin-3-yl-ethanone

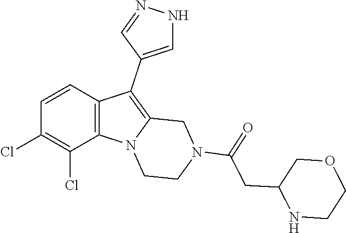 [SMILES: O=C(CC1COCCN1)N1CCn2c(c(-c3cn[nH]c3)c3ccc(Cl)c(Cl)c32)C1]

Suspend tert-butyl 3-[2-[6, 7-dichloro-10-(1H-pyrazol-4-yl)-3,4-dihydro-1H-pyrazino[1, 2-a]indol-2-yl]-2-oxo-ethyl]morpholine-4-carboxylate (290 mg, 0.516 mmol) in DCM (4 mL). Add TFA (2 mL) and stir for 1 hour at RT. Concentrate to yield 1-[6,7-dichloro-10-(1H-pyrazol-4-yl)-3,4-dihydro-1H-pyrazino[1,2-a]indol-2-yl]-2-morpholin-3-yl-ethanone (230 mg, 0.477 mmol, 92% Yield, TFA salt). ES-MS (m/z): 434.0/436.0 (M+H).

### EXAMPLE 81

### 1-[6,7-Dichloro-10-(1H-pyrazol-4-yl)-3,4-dihydro-1H-pyrazino[1,2-a]indol-2-yl]-2-(4-methylmorpholin-3-yl)ethanone

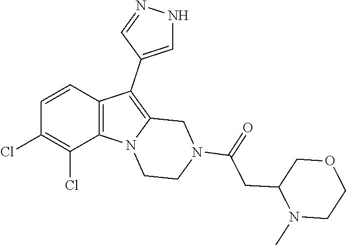 [SMILES: CN1CCOCC1CC(=O)N1CCn2c(c(-c3cn[nH]c3)c3ccc(Cl)c(Cl)c32)C1]

Suspend 1-[6, 7-dichloro-10-(1H-pyrazol-4-yl)-3, 4-dihydro-1H-pyrazino[1, 2-a]indol-2-yl]-2-morpholin-3-yl-ethanone (230 mg, 0.477 mmol) in MeOH (4 mL). Adjust to pH 8 with DIPEA and then adjust to pH 5 with AcOH. Add 37% aqueous formaldehyde (360 mg) and stir for 30 min at RT. Add NaBH₃CN (150 mg, 2.39 mmol) and stir for 2 hours at RT. Concentrate and purify by prep. HPLC to yield 1-[6,7-dichloro-10-(1H-pyrazol-4-yl)-3,4-dihydro-1H-pyrazino[1,2-a]indol-2-yl]-2-(4-methylmorpholin -3-yl)ethenone (73.71 mg, 0.16 mmol, 34% Yield, formic acid salt). ES-MS (m/z): 448.3/450.3 (M+H). ¹H NMR (400 MHz, DMSO): 8.14 (s, 0.3H, formic acid), 7.95 (br s, 0.7H, minor rotamer), 7.87 (br s, 1.3H, major rotamer), 7.66 (d, J=8.8 Hz, 0.3H, minor rotamer), 7.63 (d, J=8.6 Hz, 0.7H, major rotamer), 7.27 (d, J=8.5 Hz, 1H), 4.99 (s, 0.7H, minor rotamer), 4.90 (s, 1.3H, major rotamer), 4.75 (t, J=5.3 Hz, 1.3H, major rotamer), 4.71-4.59 (m, 0.7H, minor rotamer), 4.08-3.89 (m, 2H), 3.77-3.66 (m, 2H), 3.60-3.48 (m, 1H), 3.33-3.22 (m, 1H), 2.96-2.74 (m, 3H), 2.54-2.40 (m, 2H), 2.34 (br s, 3H).

### SCHEME FOR EXAMPLE 82

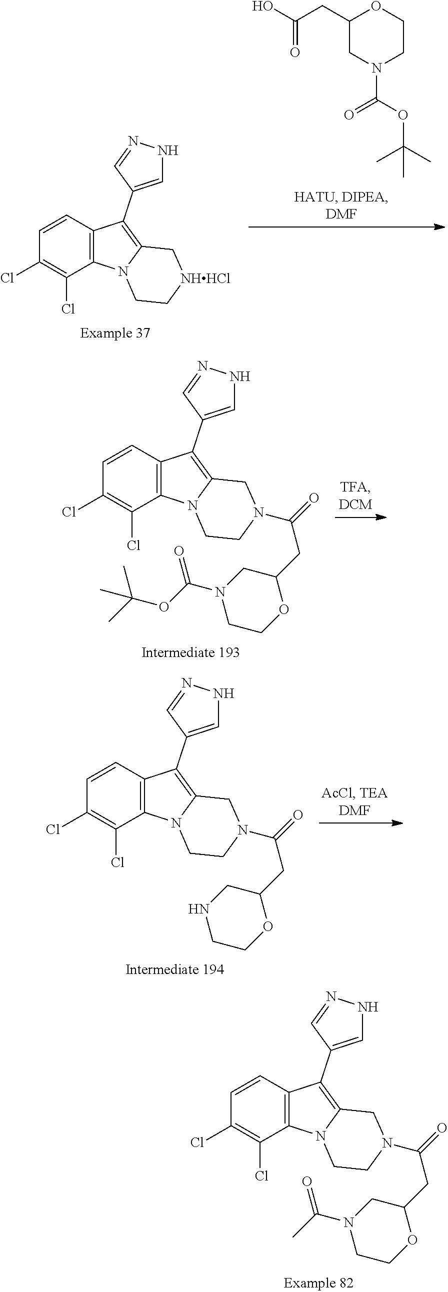

### Intermediate 193

### tert-Butyl 2-[2-[6,7-dichloro-10-(1H-pyrazol-4-yl)-3,4-dihydro-1H-pyrazino[1,2-a]indol-2-yl]-2-oxo-ethyl]morpholine-4-carboxylate

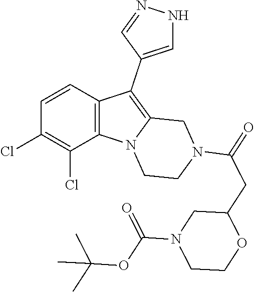 [SMILES: CC(C)(C)OC(=O)N1CCOC(CC(=O)N2CCn3c(c(-c4cn[nH]c4)c4ccc(Cl)c(Cl)c43)C2)C1]

Suspend 6, 7-dichloro-10-(1H-pyrazol-4-yl)-1, 2, 3, 4-tetrahydropyrazino[1,2-a]indole (200 mg, 0.553 mmol, HCl salt) in DMF (8 mL). Add DIPEA (215 mg, 1.66 mmol), 2-(4-tert-butoxycarbonylmorpholin-2-yl)acetic acid (150 mg, 0.612 mmol) and stir for 30 minutes at RT. Add HATU (258 mg, 0.665 mmol) and stir for 2 hours at RT. Dilute with H₂O, extract with EtOAc, wash with brine, dry over anhydrous sodium sulfate, filter, and concentrate. Add MeOH (6 mL) and K₂CO₃ (230 mg, 1.66421 mmol) to the residue and stir overnight at RT. Dilute with H₂O, extract with EtOAc, wash with brine, dry over anhydrous sodium sulfate, filter, and concentrate to yield tert-butyl 2-[2-[6, 7-dichloro-10-(1H-pyrazol-4-yl)-3, 4-dihydro-1H-pyrazino[1,2-a]indol-2-yl]-2-oxo-ethyl]morpholine-4-carboxylate (350 mg, 0.459 mmol, 83% Yield). ES-MS (m/z): 534.1/536.1 (M+H).

### Intermediate 194

### 1-[6,7-Dichloro-10-(1H-pyrazol-4-yl)-3,4-dihydro-1H-pyrazino[1,2-a]indol-2-yl]-2-morpholin-2-yl-ethanone

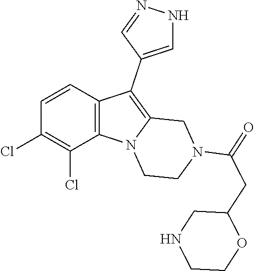 [SMILES: O=C(CC1CNCCO1)N1CCn2c(c(-c3cn[nH]c3)c3ccc(Cl)c(Cl)c32)C1]

Dissolve tert-butyl 2-[2-[6, 7-dichloro-10-(1H-pyrazol-4-yl)-3, 4-dihydro-1H-pyrazino[1, 2-a] indol-2-yl]-2-oxo-ethyl]morpholine-4-carboxylate (350 mg, 0.459 mmol) in DCM (8 mL). Add TFA (2 mL) and stir for 2 hours at RT. Concentrate to yield 1-[6,7-dichloro-10-(1H-pyrazol-4-yl)-3,4-dihydro-1H-pyrazino[1,2-a]indol-2-yl]-2-morpholin-2-yl-ethanone (220 mg, 0.4306 mmol, 94% Yield, TFA salt). ES-MS (m/z): 434.0/436.2 (M+H).

### EXAMPLE 82

### 2-(4-Acetylmorpholin-2-yl)-1-[6,7-dichloro-10-(1H-pyrazol-4-yl)-3,4-dihydro-1H-pyrazino[1,2-a]indol-2-yl]ethanone

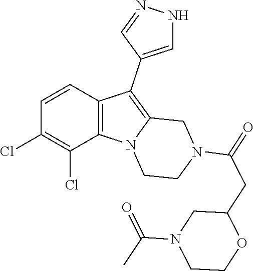 [SMILES: CC(=O)N1CCOC(CC(=O)N2CCn3c(c(-c4cn[nH]c4)c4ccc(Cl)c(Cl)c43)C2)C1]

Suspend 1-[6, 7-dichloro-10-(1H-pyrazol-4-yl)-3, 4-dihydro-1H-pyrazino[1, 2-a]indol-2-yl]-2-morpholin-2-yl-ethanone (220 mg, 0.431 mmol) in DCM (4 mL). Add Et₃N (131 mg, 1.29 mmol) and acetyl chloride (51 mg, 0.65 mmol) and stir for 2 hours at RT. Dilute with H₂O, extract with EtOAc, wash with brine, dry over anhydrous sodium sulfate, filter, and concentrate. Add MeOH (8 mL) and K₂CO₃ (179 mg, 1.30 mmol) to the residue and stir overnight at RT. Dilute with H₂O, extract with EtOAc, wash with brine, dry over anhydrous sodium sulfate, filter, and concentrate. Purify by prep. HPLC to yield 2-(4-acetylmorpholin-2-yl)-1-[6,7-dichloro-10-(1H-pyrazol-4-yl)-3,4-dihydro-1H-pyrazino[1,2-a]indol-2-yl]ethenone (77 mg, 0.16 mmol, 37% Yield). ES-MS (m/z): 476.0/478.0 (M+H). ¹H NMR (400 MHz, MeOD): 7.86 (s, 0.7H, one rotamer), 7.82 (s, 1.3 H, two overlapping rotamers), 7.57-7.45 (m, 1H), 7.25-7.13 (m, 1H), 5.13-4.91 (m, 2H), 4.85-4.65 (m, 2H), 4.49-4.18 (m, 1H), 4.15-3.99 (m, 2H), 3.99-3.33 (m, 4H), 3.30-2.93 (m, 1H), 2.92-2.47 (m, 3H), 2.11 (s, 1H, rotamer 1), 2.10 (s, 1H, rotamer 2), 2.06 (s, 1H, rotamer 3).

### SCHEME FOR EXAMPLE 83

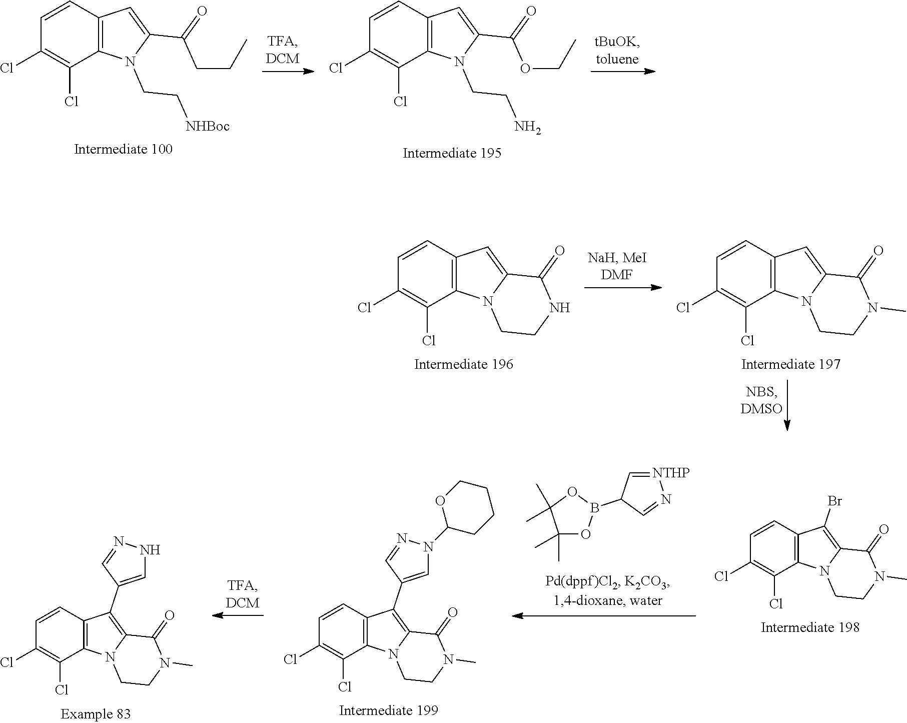

### Intermediate 195

### Ethyl 1-(2-aminoethyl)-6,7-dichloro-indole-2-carboxylate

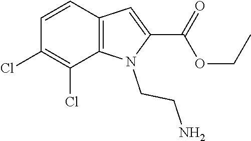 [SMILES: CCOC(=O)c1cc2ccc(Cl)c(Cl)c2n1CCN]

Dissolve ethyl 1-[2-(tert-butoxycarbonylamino)ethyl]-6,7-dichloro-indole-2-carboxylate (3.33 g, 7.88 mmol) in DCM (15 mL). Add TFA (15 mL) and stir at RT overnight. Dilute with saturated aqueous sodium bicarbonate and extract with EtOAc (3×). Wash the combined organics with brine, dry over anhydrous sodium sulfate, filter and concentrate to yield ethyl 1-(2-aminoethyl)-6,7-dichloro-indole-2-carboxylate (2.9 g, 7.7 mmol, 80 mass %, 98% Yield). ES-MS (m/z): 300.9/302.8 (M+H).

### Intermediate 196

### 6,7-Dichloro-3,4-dihydro-2H-pyrazino[1,2-a]indol-1-one

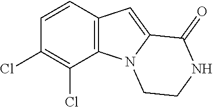 [SMILES: O=C1NCCn2c1cc1ccc(Cl)c(Cl)c12]

Dissolve ethyl 1-(2-aminoethyl)-6,7-dichloro-indole-2-carboxylate (2.9 g, 7.7 mmol, 80 mass %) in toluene (60 mL) and add t-BuOK (1.8 g, 15 mmol). Stir at 100° C. for 1 hour. Dilute with water and adjust the pH to 8 with 2 N aqueous NaOH. Extract with EtOAc, wash with brine, dry over anhydrous sodium sulfate, filter, and concentrate to yield 6,7-dichloro-3,4-dihydro-2H-pyrazino[1,2-a]indol-1-one (2.1 g, 7.4 mmol, 90 mass %, 96% Yield). ES-MS (m/z): 254.9/256.9 (M+H).

### Intermediate 197

### 6,7-Dichloro-2-methyl-3,4-dihydropyrazino[1,2-a]indol-1-one

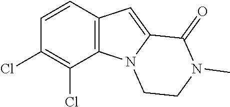 [SMILES: CN1CCn2c(cc3ccc(Cl)c(Cl)c32)C1=O]

Dissolve 6,7-dichloro-3,4-dihydro-2H-pyrazino[1,2-a]indol-1-one (300 mg, 1.06 mmol) in DMF (5 mL). Add NaH (65 mg, 1.62 mmol, 60% dispersion in mineral oil) at 0° C. and stir under nitrogen for 0.5 hour. Add methyl iodide (0.15 mL, 2.40 mmol) dropwise at 0° C. Stir 1 hour at RT. Add water, extract with EtOAc, wash with 4% aqueous LiCl, dry over anhydrous sodium sulfate, filter and concentrate to yield 6,7-dichloro-2-methyl-3,4-dihydropyrazino[1,2-a]indol-1-one (210 mg, 0.780 mmol, 74% Yield).

### Intermediate 198

### 10-Bromo-6,7-dichloro-2-methyl-3,4-dihydropyrazino[1,2-a]indol-1-one

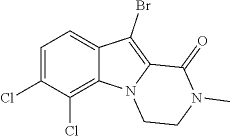 [SMILES: CN1CCn2c(c(Br)c3ccc(Cl)c(Cl)c32)C1=O]

Dissolve 6,7-dichloro-2-methyl-3,4-dihydropyrazino[1,2-a]indol-1-one (200 mg, 0.743 mmol) in DMSO (4 mL) and add NBS (200 mg, 1.10 mmol). Stir for 1 hour at RT. Filter and wash the solid with petroleum ether to yield 10-bromo-6,7-dichloro-2-methyl-3,4-dihydropyrazino[1,2-a]indol-1-one (192 mg, 0.551 mmol, 74% Yield).

### Intermediate 199

### 6,7-Dichloro-2-methyl-10-(1-tetrahydropyran-2-ylpyrazol-4-yl)-3,4-dihydropyrazino[1,2-a]indol-1-one

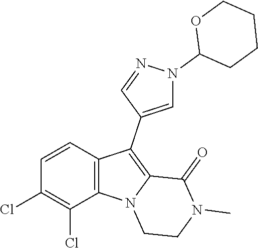 [SMILES: CN1CCn2c(c(-c3cnn(C4CCCCO4)c3)c3ccc(Cl)c(Cl)c32)C1=O]

Suspend 10-bromo-6,7-dichloro-2-methyl-3,4-dihydropyrazino[1,2-a]indol-1-one (140 mg, 0.40 mmol), 1-(tetrahydro-2H-pyran-2-yl)-4-(4,4,5,5-tetramethyl-1,3,2-dioxaborolan-2-yl)-1H-pyrazole (145 mg, 0.49 mmol), K₂CO₃ (170 mg, 1.23 mmol) and Pd(dppf)Cl₂ (30 mg, 0.04 mmol) in 1,4-dioxane (4 mL) and water (1 mL). Sparge with nitrogen and heat to 100° C. overnight. Cool to RT, dilute with EtOAc, wash sequentially with saturated aqueous sodium bicarbonate and brine, dry over anhydrous sodium sulfate, filter, and concentrate. Purify by flash column chromatography (MeOH/DCM) to yield 6,7-dichloro-2-methyl-10-(1-tetrahydropyran-2-ylpyrazol-4-yl)-3,4-dihydropyrazino[1,2-a]indol-1-one (290 mg, 0.484 mmol, 89% Yield).

### EXAMPLE 83

### 6,7-Dichloro-2-methyl-10-(1H-pyrazol-4-yl)-3,4-dihydropyrazino[1,2-a]indol-1-one

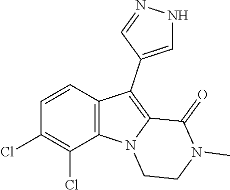 [SMILES: CN1CCn2c(c(-c3cn[nH]c3)c3ccc(Cl)c(Cl)c32)C1=O]

Suspend 6, 7-dichloro-2-methyl-10-(1-tetrahydropyran-2-ylpyrazol-4-yl)-3, 4-dihydro pyrazino[1,2-a]indol-1-one (290 mg, 0.48 mmol) in DCM (3 mL). Add TFA (1 mL) and stir for 1 hour at RT. Concentrate and dilute with water, extract with EtOAc, wash with saturated aqueous sodium bicarbonate (2×), dry over anhydrous sodium sulfate, filter, and concentrate. Purify by prep-HPLC to yield 6,7-dichloro-2-methyl-10-(1H-pyrazol-4-yl)-3,4-dihydropyrazino[1,2-a]indol-1-one (57 mg, 0.17 mmol, 34% Yield) as a white solid. ES-MS (m/z): 335.0/337.0 (M+H). ¹H NMR (400 MHz, DMSO): 12.94 (br s, 1H), 8.29-7.77 (m, 2H), 7.71 (d, J=8.6 Hz, 1H), 7.31 (d, J=8.6 Hz, 1H), 4.87 (dd, J=6.4, 5.0 Hz, 2H), 3.78 (dd, J=6.4, 5.0 Hz, 2H), 3.04 (s, 3H).

### SCHEME FOR EXAMPLE 84

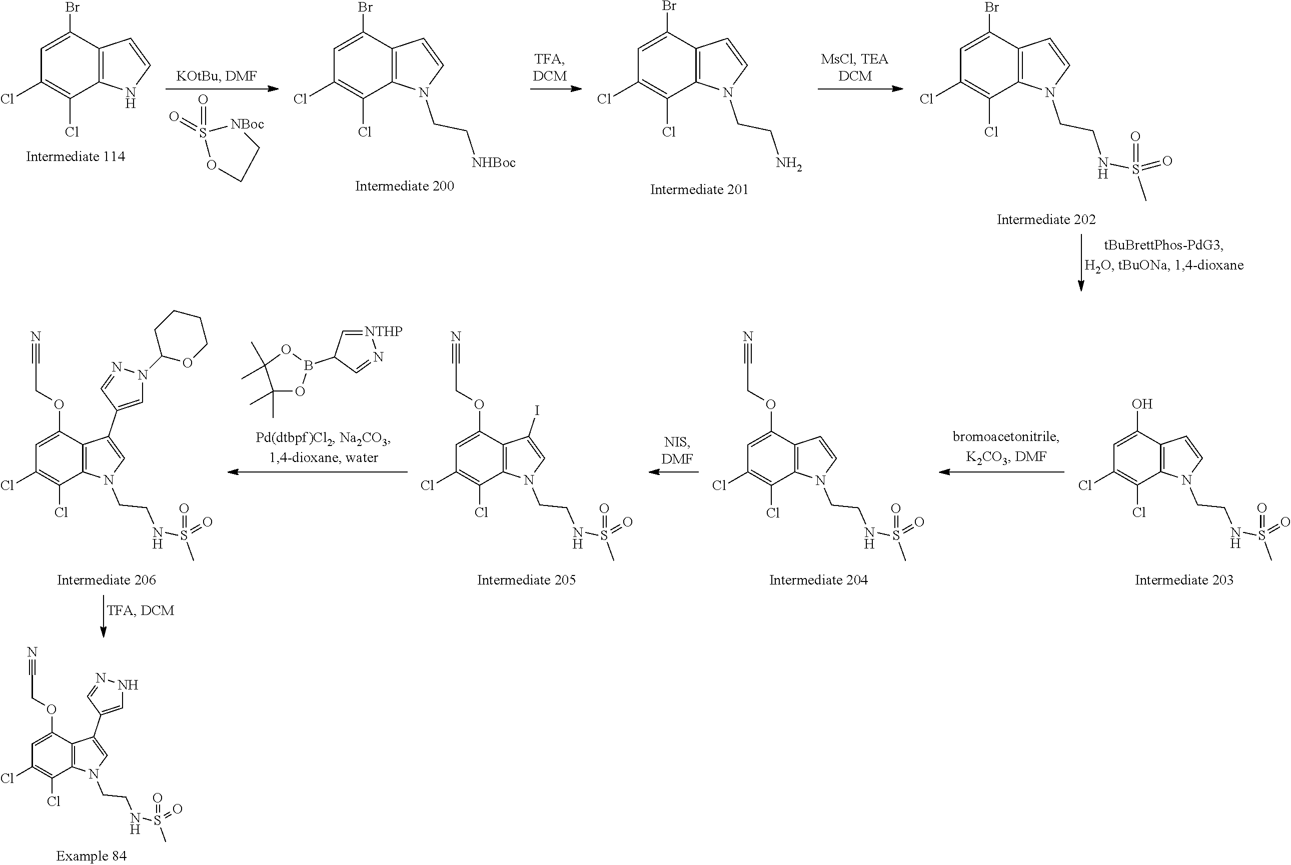

### Intermediate 200

### N-[2-(4-Bromo-6,7-dichloro-indol-1-yl)ethyl]carbamate

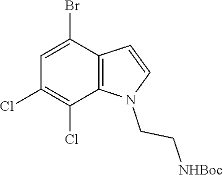 [SMILES: CC(C)(C)OC(=O)NCCn1ccc2c(Br)cc(Cl)c(Cl)c21]

Dissolve 4-bromo-6,7-dichloro-1H-indole (1.00 g, 3.59 mmol) in DMF (15 mL, 99 mass %) and add potassium tert-butoxide (1.1 g, 9.5 mmol) at 0° C. Stir for 1 hour then add tert-butyl 1,2,3-oxathiazolidine-3-carboxylate 2,2-dioxide (1.8 g, 7.7 mmol) at 0° C. Stir at RT for 3 hours. Dilute with water and extract with EtOAc. Wash sequentially with 4% LiCl and 1 M HCl, dry over anhydrous sodium sulfate, filter, and concentrate. Yields tert-butyl N-[2-(4-bromo-6,7-dichloro-indol-1-yl)ethyl]carbamate (1.2 g, 2.6 mmol, 90 mass %, 74% Yield).

### Intermediate 201

### 2-(4-Bromo-6,7-dichloro-indol-1-yl)ethanamine

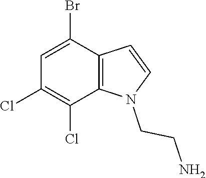 [SMILES: NCCn1ccc2c(Br)cc(Cl)c(Cl)c21]

Dissolve tert-butyl N-[2-(4-bromo-6,7-dichloro-indol-1-yl)ethyl]carbamate (1.2 g, 2.6 mmol, 90 mass %) in TFA (3 mL) and DCM (9 mL). Stir at RT for 1 hour. Quench with saturated aqueous sodium bicarbonate and extract with DCM. Dry over anhydrous sodium sulfate, filter, and concentrate. Yields 2-(4-bromo-6,7-dichloro-indol-1-yl)ethanamine (890 mg, 2.75 mmol, 95 mass %, 100% Yield).

### Intermediate 202

### N-[2-(4-Bromo-6, 7-dichloro-indol-1-yl)ethyl]methanesulfonamide

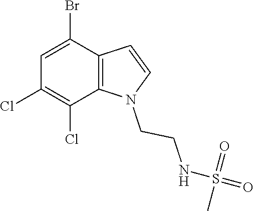 [SMILES: CS(=O)(=O)NCCn1ccc2c(Br)cc(Cl)c(Cl)c21]

Suspend 2-(4-bromo-6,7-dichloro-indol-1-yl)ethanamine (790 mg, 2.44 mmol) and Et₃N (740 mg, 7.31 mmol) in DCM (5 mL) at 0° C. Add methanesulfonyl chloride (400 mg, 3.49 mmol) and stir for 2 hours at 0° C. Quench with saturated aqueous NaHCO₃, extract with EtOAc, wash with brine, dry over anhydrous sodium sulfate, filter, and concentrate. Purify by flash column chromatography (EtOAc/petroleum ether) to yield N-[2-(4-bromo-6, 7-dichloro-indol-1-yl)ethyl]methane sulfonamide (600 mg, 1.48 mmol, 61% Yield).

### Intermediate 203

### N-[2-(6,7-Dichloro-4-hydroxy-indol-1-yl)ethyl]methanesulfonamide

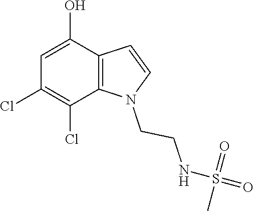 [SMILES: CS(=O)(=O)NCCn1ccc2c(O)cc(Cl)c(Cl)c21]

Suspend N-[2-(4-bromo-6,7-dichloro-indol-1-yl)ethyl]methanesulfonamide (570 mg, 1.40 mmol) in 1,4-dioxane (8 mL) and H₂O (2 mL). Add sodium tert-butoxide (350 mg, 3.53 mmol) and t-BuBrettPhos-Pd-G₃ (123 mg, 0.141 mmol). Sparge with nitrogen and stir for 3 hours at 65° C. Quench with saturated aqueous NH₄Cl and extract with EtOAc. Dry over anhydrous sodium sulfate, filter, and concentrate. Purify by flash column chromatography (EtOAc/PE) to yield N-[2-(6,7-dichloro-4-hydroxy-indol-1-yl)ethyl]methane sulfonamide (330 mg, 0.970 mmol, 69% Yield).

### Intermediate 204

### N-[2-[6,7-Dichloro-4-(cyanomethoxy)indol-1-yl]ethyl]methanesulfonamide

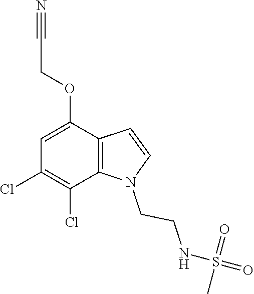 [SMILES: CS(=O)(=O)NCCn1ccc2c(OCC#N)cc(Cl)c(Cl)c21]

Suspend N-[2-(6,7-dichloro-4-hydroxy-indol-1-yl)ethyl]methanesulfonamide (300 mg, 0.882 mmol) in DMF (8 mL). Add K₂CO₃ (122 mg, 0.883 mmol), bromoacetonitrile (212 mg, 1.77 mmol) and stir for 3 hours at RT. Quench with H₂O, extract with EtOAc, wash with brine, dry over anhydrous sodium sulfate, filter, and concentrate. Purify by flash column chromatography (EtOAc/petroleum ether) to yield N-[2-[6,7-dichloro-4-(cyanomethoxy)indol-1-yl]ethyl]methanesulfonamide (300 mg, 0.580 mmol, 66% Yield). ES-MS (m/z): 362.0/364.0 (M+H).

### Intermediate 205

### N-[2-[6,7-Dichloro-4-(cyanomethoxy)-3-iodo-indol -1-yl]ethyl]methanesulfonamide

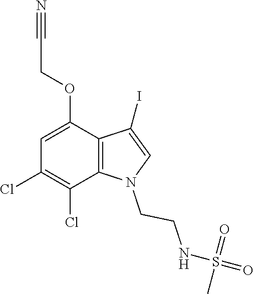 [SMILES: CS(=O)(=O)NCCn1cc(I)c2c(OCC#N)cc(Cl)c(Cl)c21]

Suspend N-[2-[6, 7-dichloro-4-(cyanomethoxy)indol-1-yl]ethyl]methanesulfonamide (270 mg, 0.522 mmol, 70% purity) in DMF (4 mL). Add NIS (144 mg, 0.627 mmol) and stir 1.5 hours at RT. Dilute with EtOAc, wash with saturated aqueous Na₂SO₃, brine, dry over anhydrous sodium sulfate, filter, and concentrate. Purify by flash column chromatography (EtOAc/petroleum ether) to yield N-[2-[6,7-dichloro-4-(cyanomethoxy)-3-iodo-indol-1-yl]ethyl]methanesulfonamide (270 mg, 0.498 mmol, 95% Yield).

### Intermediate 206

### N-[2-[6,7-Dichloro-4-(cyanomethoxy)-3-(1-tetrahydropyran-2-ylpyrazol-4-yl)indol-1-yl]ethyl]methanesulfonamide

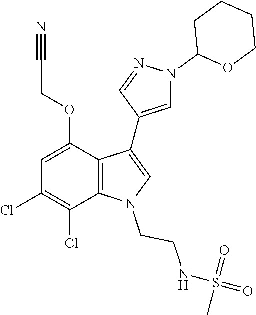 [SMILES: CS(=O)(=O)NCCn1cc(-c2cnn(C3CCCCO3)c2)c2c(OCC#N)cc(Cl)c(Cl)c21]

Suspend N-[2-[6, 7-dichloro-4-(cyanomethoxy)-3-iodo-indol-1-yl]ethyl]methanesulfonamide (250 mg, 0.461 mmol) in 1,4-dioxane (4 mL) and H₂O (1 mL). Add 1-(tetrahydro-2h-pyran-2-yl)-4-(4,4,5,5-tetramethyl-1,3,2-dioxaborolan-2-yl)-1h-pyrazole (203 mg, 0.693 mmol), Pd(dtbpf)Cl₂ (31 mg, 0.047 mmol), Na₂CO₃ (147 mg, 1.39 mmol). Sparge with nitrogen and stir 1 hour at 90° C. Dilute with H₂O and extract with EtOAc, wash with brine, dry over anhydrous sodium sulfate, filter, and concentrate. Purify by flash column chromatography (MeOH/DCM) to yield N-[2-[6, 7-dichloro-4-(cyanomethoxy)-3-(1-tetrahydropyran-2-ylpyrazol-4-yl)indol-1-yl]ethyl]methanesulfonamide (360 mg, 0.422 mmol, 91% Yield). ES-MS (m/z): 512.1/514.1 (M+H).

### EXAMPLE 84

### N-[2-[6,7-Dichloro-4-(cyanomethoxy)-3-(1H-pyrazol-4-yl)indol-1-yl]ethyl]methanesulfonamide

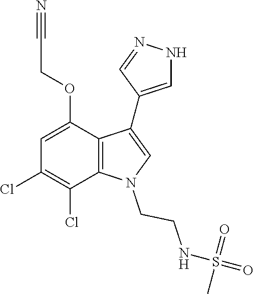 [SMILES: CS(=O)(=O)NCCn1cc(-c2cn[nH]c2)c2c(OCC#N)cc(Cl)c(Cl)c21]

Suspend N-[2-[6, 7-dichloro-4-(cyanomethoxy)-3-(1-tetrahydropyran-2-ylpyrazol-4-yl) indol-1-yl]ethyl]methanesulfonamide (360 mg, 0.422 mmol) in DCM (3 mL). Add TFA (3 mL) and stir for 1 hour at RT. Concentrate and purify by prep-HPLC to yield N-[2-[6, 7-dichloro-4-(cyanomethoxy)-3-(1H-pyrazol-4-yl)indol-1-yl]ethyl]methane sulfonamide (85.19 mg, 0.195 mmol, 46% Yield). ES-MS (m/z): 428.0/430.0 (M+H). ¹H NMR (400 MHz, DMSO): 7.77 (s, 2H), 7.51 (s, 1H), 7.30 (t, J=5.9 Hz, 1H), 7.02 (s, 1H), 5.29 (s, 2H), 4.60 (t, J=6.4 Hz, 2H), 3.38 (q, J=6.3 Hz, 2H), 2.83 (s, 3H).

### SCHEME FOR EXAMPLE 85

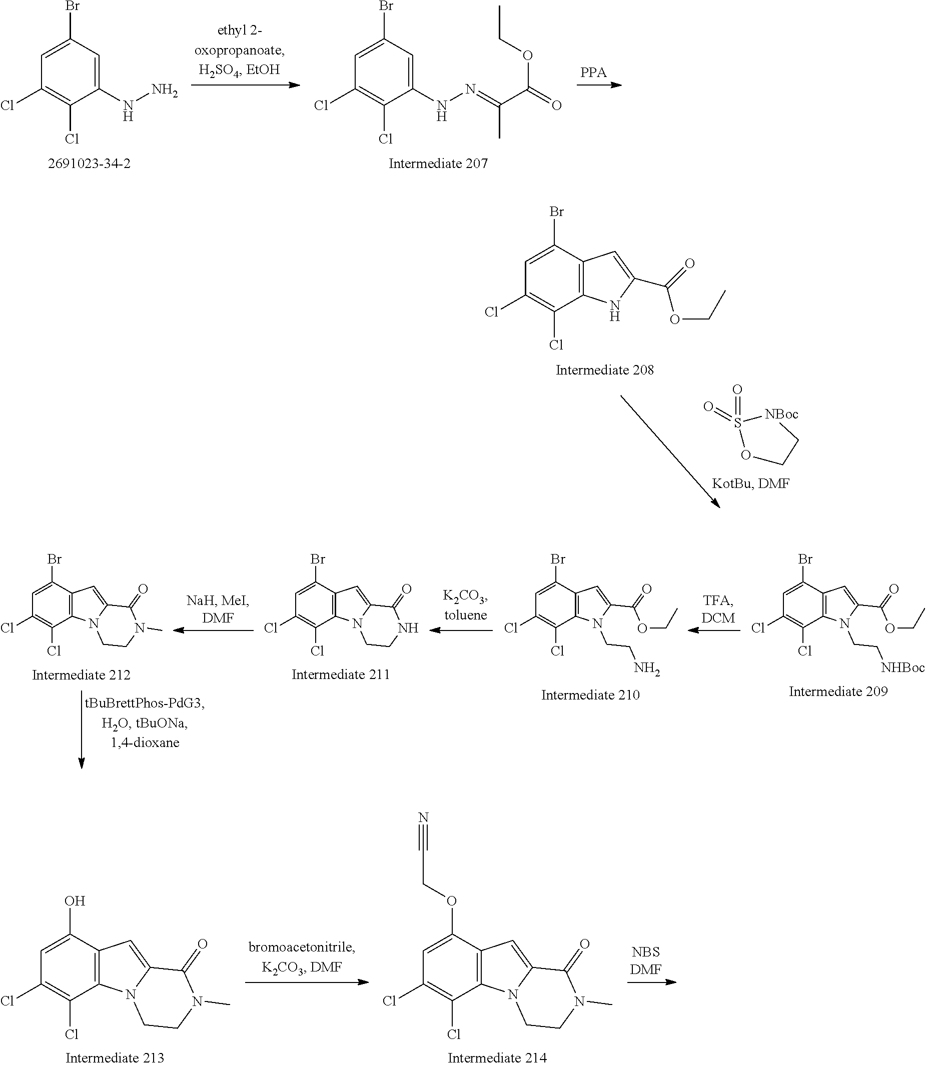

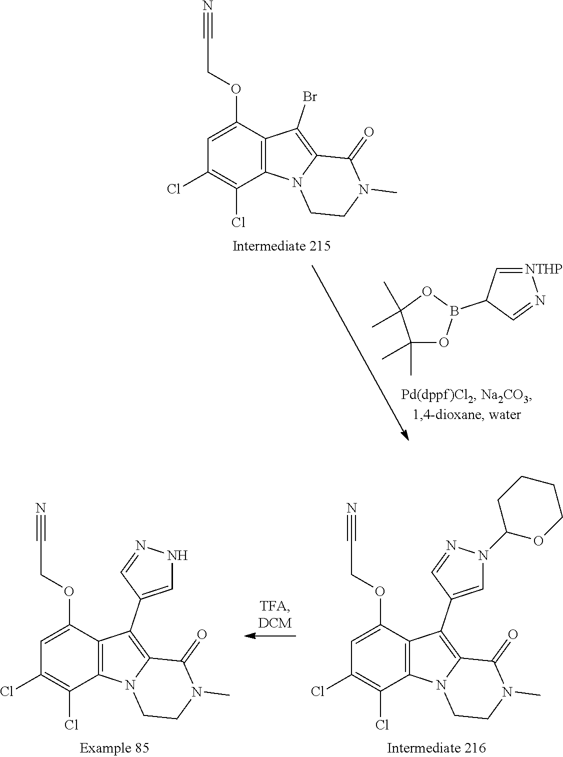

### Intermediate 207

### Ethyl (2E)-2-[(5-bromo-23-dichloro-phenyl)hydrazono]propanoate

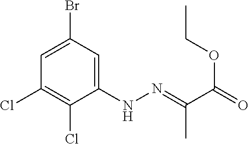 [SMILES: CCOC(=O)/C(C)=N/Nc1cc(Br)cc(Cl)c1Cl]

Dissolve (5-bromo-2, 3-dichlorophenyl) hydrazine (7.50 g, 29.0 mmol) in conc. sulfuric acid (6.4 mL) and ethanol (30 mL) at 0° C. Add ethyl 2-oxopropanoate (3.40 g, 29.0 mmol) in ethanol (11 mL) at 0° C. Stir for 2 hours at 0° C., then heat to 45° C. for 16 hours. Cool to RT and add cold EtOH, collect the precipitate by filtration and dry to yield ethyl (2E)-2-[(5-bromo-2,3-dichloro-phenyl)hydrazono]propanoate (8.30 g, 22.0 mmol, 76% Yield).

### Intermediate 208

### Ethyl 4-bromo-6,7-dichloro-1H-indole-2-carboxylate

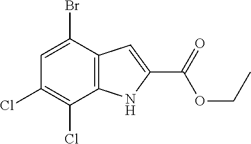 [SMILES: CCOC(=O)c1cc2c(Br)cc(Cl)c(Cl)c2[nH]1]

Dissolve ethyl-2-(2-(5-bromo-2, 3-dichlorophenyl)hydrazono)propanoate (8.30 g, 22 mmol) in PPA (80 mL). Stir for 2 hours at RT. Pour into ice water and stir vigorously for 15 min. Extract with EtOAc, wash with brine, dry over anhydrous sodium sulfate, filter, and concentrate. Purify by flash column chromatography (ethyl acetate/petroleum ether) to yield ethyl 4-bromo-6, 7-dichloro-1H-indole-2-carboxylate (1.80 g, 5.07 mmol, 23% Yield).

### Intermediate 209

### Ethyl 4-bromo-1-[2-(tert-butoxycarbonylamino)ethyl]-6, 7-dichloro-indole-2-carboxylate

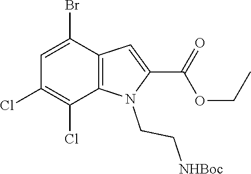 [SMILES: CCOC(=O)c1cc2c(Br)cc(Cl)c(Cl)c2n1CCNC(=O)OC(C)(C)C]

Suspend ethyl 4-bromo-6, 7-dichloro-1H-indole-2-carboxylate (2.30 g, 6.50 mmol) in DMF (50 mL). Add potassium tert-butoxide (980 mg, 8.47 mmol) at 0° C. and stir for 1 hour. Add tert-butyl 1, 2, 3-oxathiazolidine-3-carboxylate 2,2-dioxide (2.30 g, 9.80 mmol). Stir at RT overnight. Dilute with EtOAc, wash with brine, dry over anhydrous sodium sulfate, filter, and concentrate. Purify by flash column chromatography (EtOAc/petroleum ether) to yield ethyl 4-bromo-1-[2-(tert-butoxycarbonylamino) ethyl]-6, 7-dichloro-indole-2-carboxylate (1.70 g, 2.83 mmol, 44% Yield).

### Intermediate 210

### Ethyl 1-(2-aminoethyl)-4-bromo-6, 7-dichloro-1H-indole-2-carboxylate

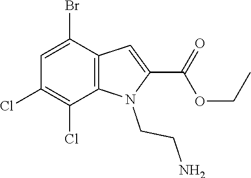 [SMILES: CCOC(=O)c1cc2c(Br)cc(Cl)c(Cl)c2n1CCN]

Dissolve ethyl 4-bromo-1-[2-(tert-butoxycarbonylamino)ethyl]-6, 7-dichloro-indole-2-carboxylate (1.70 g, 2.83 mmol) in TFA (10 mL) and DCM (10 mL). Stir for 1 hour at RT. Quench with saturated aqueous sodium bicarbonate and stir vigorously for 15 min. Extract with DCM, wash with brine, dry over anhydrous sodium sulfate, filter, and concentrate. Purify by flash column chromatography (EtOAc/petroleum ether) to yield ethyl 1-(2-aminoethyl)-4-bromo-6, 7-dichloro-1H-indole-2-carboxylate (1.40 g, 2.90 mmol, 100% Yield).

### Intermediate 211

### 9-Bromo-6, 7-dichloro-3, 4-dihydropyrazino[1, 2-a] indol-1(2H)-one

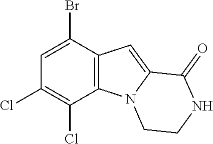 [SMILES: O=C1NCCn2c1cc1c(Br)cc(Cl)c(Cl)c12]

Dissolve ethyl 1-(2-aminoethyl)-4-bromo-6, 7-dichloro-1H-indole-2-carboxylate (1.40 g, 2.90 mmol) in toluene (20 mL). Add potassium carbonate (810 mg, 5.90 mmol). Stir for 16 hours at 100° C. Cool to RT, dilute with DCM, wash with brine, dry over sodium sulfate, filter, and concentrate. Purify by flash column chromatography (EtOAc/petroleum ether) to yield 9-bromo-6, 7-dichloro-3, 4-dihydropyrazino[1, 2-a] indol-1(2H)-one (1.10 g, 2.90 mmol, 100% Yield).

### Intermediate 212

### 9-Bromo-6, 7-dichloro-2-methyl-3, 4-dihydropyrazino[1, 2-a] indol-1(2H)-one

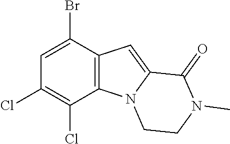 [SMILES: CN1CCn2c(cc3c(Br)cc(Cl)c(Cl)c32)C1=O]

Dissolve ethyl 9-bromo-6, 7-dichloro-3, 4-dihydropyrazino[1,2-a]indol-1(2H)-one (410 mg, 1.10 mmol) in DMF (10 mL). Add sodium hydride (133 mg, 3.30 mmol, 60% dispersion in mineral oil) and stir at 0° C. for 30 minutes. Then add methyl iodide (480 mg, 3.30 mmol). Stir at 0° C. for 1 hour. Quench with water and stir vigorously for 15 min. Dilute with EtOAc, wash with brine, dry over sodium sulfate, filter, and concentrate. Purify by flash column chromatography EtOAc/petroleum ether) to yield 9-bromo-6, 7-dichloro-2-methyl-3, 4-dihydropyrazino[1,2-a] indol-1(2H)-one (470 mg, 0.88 mmol, 80% Yield).

### Intermediate 213

### 6, 7-Dichloro-9-hydroxy-2-methyl-3,4-dihydropyrazino[1,2-a]indol-1(2H)-one

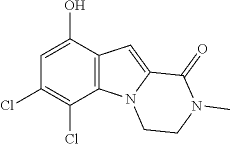 [SMILES: CN1CCn2c(cc3c(O)cc(Cl)c(Cl)c32)C1=O]

Dissolve 9-bromo-6, 7-dichloro-2-methyl-3, 4-dihydropyrazino[1, 2-a] indol-1(2H)-one (470 mg, 0.880 mmol) in 1, 4-dioxane (20 mL) and water (5 mL). Add sodium tert-butoxide (180 mg, 1.81 mmol) and t-BuBrettPhos-Pd-G₃ (63 mg, 0.070 mmol). Sparge with nitrogen and heat to 65° C. for 3 hours. Quench with water and extract with EtOAc. Wash with brine, dry over anhydrous sodium sulfate, filter and concentrate. Purify by flash column chromatography (EtOAc/petroleum ether) to yield 6, 7-dichloro-9-hydroxy-2-methyl-3,4-dihydropyrazino[1,2-a]indol-1(2H)-one (380 mg, 0.800 mmol, 91% Yield). ES-MS (m/z): 285.0/287.0 (M+H).

### Intermediate 214

### 2-((6, 7-Dichloro-2-methyl-1-oxo-1, 2, 3, 4-tetrahydropyrazino[1, 2-a] indol-9-yl) oxy) acetonitrile

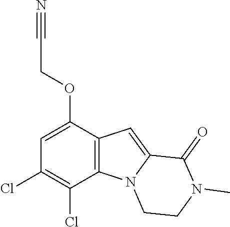 [SMILES: CN1CCn2c(cc3c(OCC#N)cc(Cl)c(Cl)c32)C1=O]

Dissolve 6, 7-dichloro-9-hydroxy-2-methyl-3, 4-dihydropyrazino[1,2-a]indol-1(2H)-one (380 mg, 1.20 mmol) in DMF (10 mL). Add bromoacetonitrile (290 mg, 2.42 mmol) and potassium carbonate (220 mg, 1.60 mmol). Stir for 4 hours at RT. Add water and extract with EtOAc. Wash with brine, dry over sodium sulfate, filter, and concentrate. Purify by flash column chromatography (EtOAc/petroleum ether) to yield 2-((6, 7-dichloro-2-methyl-1-oxo-1, 2, 3, 4-tetrahydropyrazino[1, 2-a] indol-9-yl) oxy) acetonitrile (220 mg, 0.61 mmol, 51% Yield). ES-MS (m/z): 324.0/326.0 (M+H).

### Intermediate 215

### 2-((10-Bromo-6, 7-dichloro-2-methyl-1-oxo-1, 2, 3, 4-tetrahydropyrazino[1, 2-a] indol-9-yl) oxy) acetonitrile

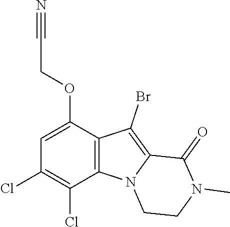 [SMILES: CN1CCn2c(c(Br)c3c(OCC#N)cc(Cl)c(Cl)c32)C1=O]

Dissolve 2-((6, 7-dichloro-2-methyl-1-oxo-1, 2, 3, 4-tetrahydropyrazino[1, 2-a] indol-9-yl) oxy) acetonitrile (220 mg, 0.610 mmol) in DMF (10 mL). Add NBS (130 mg, 0.730 mmol). Stir for 2 hours at RT. Quench with water and stir vigorously for 15 min. Collect the precipitate by suction filtration and dry to yield 2-((10-bromo-6,7-dichloro-2-methyl-1-oxo-1,2,3,4-tetrahydropyrazino[1,2-a]indol-9-yl)oxy)aceto nitrile (220 mg, 0.48 mmol, 79% Yield). ES-MS (m/z): 402.0/404.0/405.9 (M+H).

### Intermediate 216

### 2-((6, 7-Dichloro-2-methyl-1-oxo-10-(1-(tetrahydro-2H-pyran-2-yl)-1H-pyrazol-4-yl)-1, 2, 3, 4-tetrahydropyrazino[1, 2-a] indol-9-yl) oxy) acetonitrile

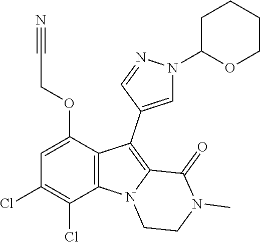 [SMILES: CN1CCn2c(c(-c3cnn(C4CCCCO4)c3)c3c(OCC#N)cc(Cl)c(Cl)c32)C1=O]

Dissolve 2-((10-bromo-6, 7-dichloro-2-methyl-1-oxo-1, 2, 3, 4-tetrahydropyrazino[1,2-a]indol-9-yl)oxy)acetonitrile (200 mg, 0.440 mmol) and 1-(tetrahydro-2H-pyran-2-yl)-4-(4, 4, 5, 5-tetramethyl-1, 3, 2-dioxaborolan-2-yl)-1H-pyrazole (154 mg, 0.530 mmol) in 1,4-dioxane (10 mL) and water (2.5 mL). Add Pd(dppf)Cl₂ (35 mg, 0.050 mmol) and sodium carbonate (93 mg, 0.88 mmol). Stir for 2 hours at 90° C. Concentrate and purify by flash column chromatography (EtOAc/petroleum ether) to yield 2-((6,7-dichloro-2-methyl-1-oxo-10-(1-(tetrahydro-2H-pyran-2-yl)-1H-pyrazol-4-yl)-1, 2, 3, 4-tetrahydropyrazino[1,2-a]indol-9-yl)oxy)acetonitrile (150 mg, 0.30 mmol, 67% Yield). ES-MS (m/z): 474.1/476.2 (M+H).

### EXAMPLE 85

### 2-((6, 7-Dichloro-2-methyl-1-oxo-10-(1H-pyrazol-4-yl)-1, 2, 3, 4-tetrahydropyrazino[1,2-a]indol-9-yl)oxy)acetonitrile

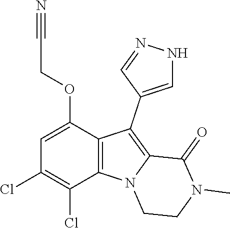 [SMILES: CN1CCn2c(c(-c3cn[nH]c3)c3c(OCC#N)cc(Cl)c(Cl)c32)C1=O]

Suspend 2-((6, 7-dichloro-2-methyl-1-oxo-10-(1-(tetrahydro-2H-pyran-2-yl)-1H-pyrazol-4-yl)-1, 2, 3, 4-tetrahydropyrazino[1,2-a]indol-9-yl)oxy)acetonitrile (130 mg, 0.250 mmol) in DCM (4 mL). Add TFA (2 mL) and stir for 1 hour at RT. Concentrate and purify by prep-HPLC to yield 2-((6, 7-dichloro-2-methyl-1-oxo-10-(1H-pyrazol-4-yl)-1, 2, 3, 4-tetrahydropyrazino[1,2-a]indol-9-yl)oxy)acetonitrile (17 mg, 0.043 mmol, 17% Yield) as a white solid. ES-MS (m/z): 390.0/392.0 (M+H). ¹H NMR (400 MHz, DMSO): 12.65 (br s, 1H), 7.84-7.35 (m, 2H), 7.02 (s, 1H), 5.13 (s, 2H), 4.86 (dd, J=6.4, 5.0 Hz, 2H), 3.74 (dd, J=6.4, 5.0 Hz, 2H), 2.99 (s, 3H).

### SCHEME FOR EXAMPLE 86

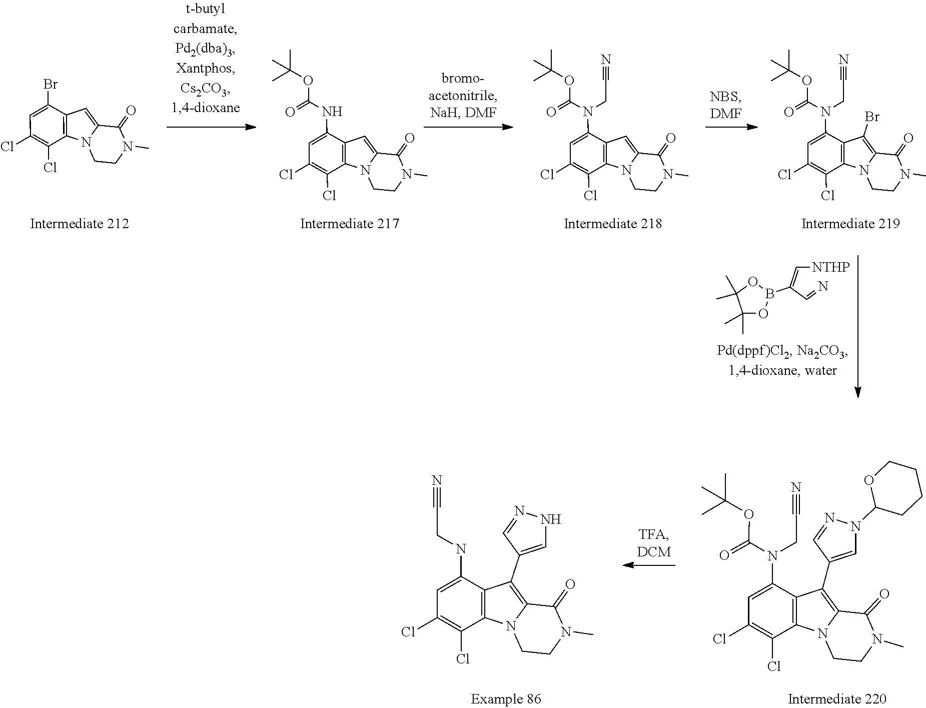

### Intermediate 217

### tert-Butyl N-(6,7-dichloro-2-methyl-1-oxo-3,4-dihydropyrazino[1,2-a]indol-9-yl)carbamate

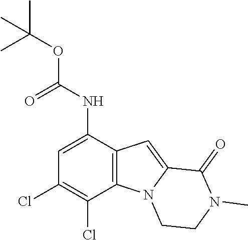 [SMILES: CN1CCn2c(cc3c(NC(=O)OC(C)(C)C)cc(Cl)c(Cl)c32)C1=O]

Dissolve 9-bromo-6, 7-dichloro-2-methyl-3,4-dihydropyrazino[1,2-a]indol-1-one (210 mg, 0.54 mmol), tert-butyl-carbamate (156 mg, 1.33 mmol), Pd₂(dba)₃ (56 mg, 0.050 mmol), Xantphos (70 mg, 0.13 mmol) and Cs₂CO₃ (500 mg, 1.53 mmol) in 1,4-dioxane (10 mL). Sparge with nitrogen and heat to 100° C. for 3 hours. Quench with water and extract with EtOAc, wash with brine, dry over anhydrous sodium sulfate, filter, and concentrate. Purify by flash column chromatography (EtOAc/petroleum ether) to yield tert-butyl N-(6, 7-dichloro-2-methyl-1-oxo-3, 4-dihydropyrazino[1,2-a]indol-9-yl)carbamate (240 mg, 80% purity, 91% Yield).

### Intermediate 218

### tert-Butyl N-(cyanomethyl)-N-(6,7-dichloro-2-methyl-1-oxo-3,4-dihydropyrazino[1,2-a]indol-9-yl)carbamate

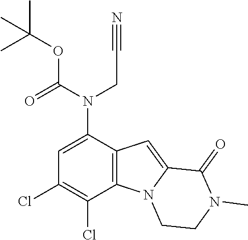 [SMILES: CN1CCn2c(cc3c(N(CC#N)C(=O)OC(C)(C)C)cc(Cl)c(Cl)c32)C1=O]

Dissolve tert-butyl-N-(6, 7-dichloro-2-methyl -1-oxo-3, 4-dihydropyrazino[1, 2-a]indol-9-yl) carbamate (240 mg, 0.620 mmol, 80% purity) in DMF (10 mL). Add NaH (40 mg, 1.0 mmol, 60% dispersion in mineral oil) and stir at 0° C. for 30 minutes. Add bromoacetonitrile (113 mg, 0.940 mmol) and stir at RT for 1 hour. Quench with water and extract with EtOAc, wash with brine, dry over anhydrous sodium sulfate, filter, and concentrate. Purify by flash column chromatography (EtOAc/petroleum ether) to yield tert-butyl N-(cyanomethyl)-N-(6, 7-dichloro-2-methyl-1-oxo-3, 4-dihydropyrazino[1,2-a]indol-9-yl)carbamate (200 mg, 0.430 mmol, 68% Yield).

### Intermediate 219

### tert-Butyl N-(10-bromo-6, 7-dichloro-2-methyl-1-oxo-3,4-dihydropyrazino[1,2-a]indol -9-yl)-N-(cyanomethyl)carbamate

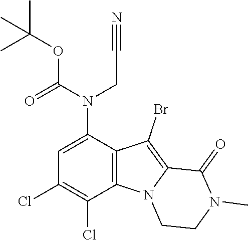 [SMILES: CN1CCn2c(c(Br)c3c(N(CC#N)C(=O)OC(C)(C)C)cc(Cl)c(Cl)c32)C1=O]

Dissolve tert-butyl N-(cyanomethyl)-N-(6, 7-dichloro-2-methyl-1-oxo-3, 4-dihydropyrazino[1,2-a]indol-9-yl)carbamate (140 mg, 0.290 mmol) in DMF (10 mL). Add NBS (92 mg, 0.50 mmol) and stir at RT for 1 hour. Dilute with H₂O and collect the precipitate by suction filtration to yield tert-butyl N-(10-bromo-6,7-dichloro-2-methyl-1-oxo-3,4-dihydropyrazino[1,2-a]indol-9-yl)-N-(cyanomethyl)carbamate (150 mg, 0.240 mmol, 80% Yield).

### Intermediate 220

### tert-Butyl N-(cyanomethyl)-N-[6,7-dichloro-2-methyl-1-oxo-10-(1-tetrahydropyran-2-ylpyrazol-4-yl)-3,4-dihydropyrazino[1,2-a]indol-9-yl]carbamate

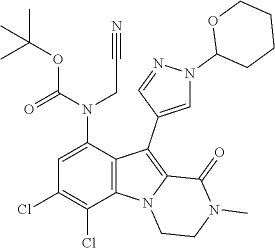 [SMILES: CN1CCn2c(c(-c3cnn(C4CCCCO4)c3)c3c(N(CC#N)C(=O)OC(C)(C)C)cc(Cl)c(Cl)c32)C1=O]

Dissolve tert-butyl N-(10-bromo-6,7-dichloro-2-methyl-1-oxo-3,4-dihydropyrazino[1,2-a]indol-9-yl)-N-(cyanomethyl)carbamate (100 mg, 0.180 mmol) and 1-(tetrahydro-2H-pyran-2-yl)-4-(4,4,5,5-tetramethyl-1,3,2-dioxaborolan-2-yl)-1H-pyrazole (120 mg, 0.410 mmol) in 1,4-dioxane (10 mL) and water (2.5 mL). Add Pd(dppf)Cl₂ (15 mg, 0.020 mmol) and sodium carbonate (42 mg, 0.40 mmol). Stir for 4 hours at 90° C. Concentrate and purify by flash column chromatography (EtOAc/petroleum ether) to yield 2-((6, 7-dichloro-2-methyl-1-oxo-10-(1-(tetrahydro-2H-pyran-2-yl)-1H-pyrazol-4-yl)-1, 2, 3, 4-tetrahydropyrazino[1,2-a]indol-9-yl)oxy)acetonitrile (60 mg, 0.090 mmol, 51% Yield). ES-MS (m/z): 573.2/575.1 (M+H).

### EXAMPLE 86

### 2-[[6,7-Dichloro-2-methyl-1-oxo-10-(1H-pyrazol-4-yl)-3,4-dihydropyrazino[1,2-a]indol-9-yl]amino]acetonitrile

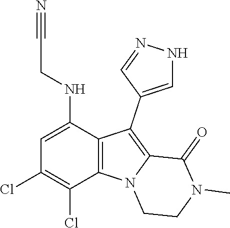 [SMILES: CN1CCn2c(c(-c3cn[nH]c3)c3c(NCC#N)cc(Cl)c(Cl)c32)C1=O]

Suspend tert-butyl N-(cyanomethyl)-N-[6, 7-dichloro-2-methyl-1-oxo-10-(1-tetrahydropyran-2-ylpyrazol-4-yl)-3, 4-dihydropyrazino[1,2-a]indol-9-yl]carbamate (60 mg, 0.10 mmol) in DCM (4 mL). Add TFA (2 mL) and stir for 2 hours at RT. Concentrate and purify by prep-HPLC to yield 2-[[6, 7-dichloro-2-methyl-1-oxo-10-(1H-pyrazol-4-yl)-3, 4-dihydropyrazino[1,2-a]indol-9-yl]amino]acetonitrile (5.50 mg, 0.014 mmol, 15% Yield). ¹H NMR (400 MHz, DMSO): 12.95 (br s, 1H), 7.75 (br s, 1H), 7.47 (br s, 1H), 6.55 (s, 1H), 5.02 (t, J=6.7 Hz, 1H), 4.88-4.78 (m, 2H), 4.32 (d, J=6.8 Hz, 2H), 3.77-3.69 (m, 2H), 2.94 (s, 3H).

### SCHEME FOR EXAMPLE 87

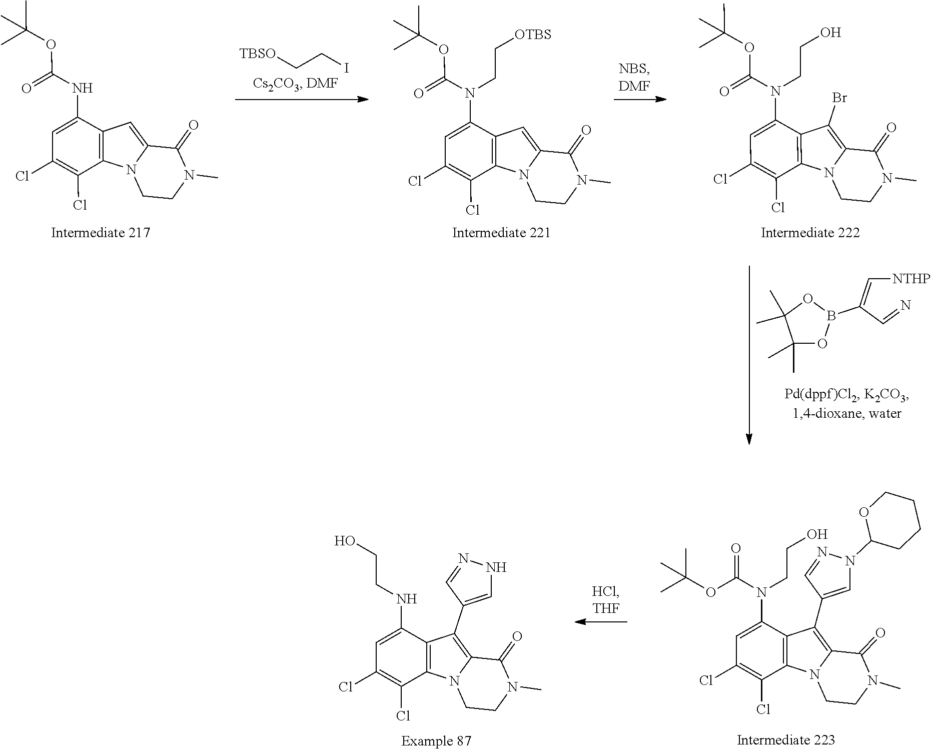

### Intermediate 221

### tert-Butyl (2-((tert-butyldimethylsilyl)oxy)ethyl)(6,7-dichloro-2-methyl-1-oxo-1,2,3,4-tetrahydropyrazino[1,2-a]indol-9-yl)carbamate

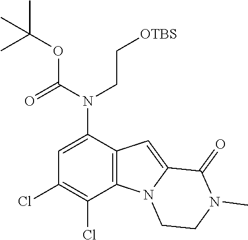 [SMILES: CN1CCn2c(cc3c(N(CCO[Si](C)(C)C(C)(C)C)C(=O)OC(C)(C)C)cc(Cl)c(Cl)c32)C1=O]

Dissolve tert-butyl (6, 7-dichloro-2-methyl-1-oxo-1, 2, 3, 4-tetrahydropyrazino[1,2-a] indol-9-yl) carbamate (350 mg, 0.865 mmol) in DMF (5 mL). Add cesium carbonate (425 mg, 1.30 mmol) and tert-butyl(2-iodoethoxy)dimethylsilane (520 mg, 1.73 mmol) at RT. Stir for 2 hours at 60° C. under nitrogen. Quench with water, wash with 4% aqueous LiCl, extract with EtOAc, dry over anhydrous sodium sulfate, filter, and concentrate. Purify by flash column chromatography (EtOAc/petroleum ether) to yield tert-butyl (2-((tert-butyldimethylsilyl)oxy)ethyl)(6, 7-dichloro-2-methyl-1-oxo-1, 2, 3, 4-tetrahydropyrazino[1,2-a]indol-9-yl)carbamate (367 mg, 0.642 mmol, 74% Yield).

### Intermediate 222

### tert-Butyl (10-bromo-6,7-dichloro-2-methyl-1-oxo-1,2,3,4-tetrahydropyrazino[1,2-a]indol-9-yl)(2-hydroxyethyl)carbamate

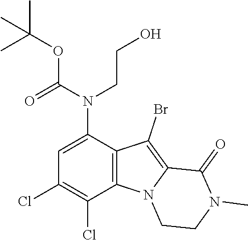 [SMILES: CN1CCn2c(c(Br)c3c(N(CCO)C(=O)OC(C)(C)C)cc(Cl)c(Cl)c32)C1=O]

Dissolve tert-butyl (2-((tert-butyldimethylsilyl)oxy)ethyl)(6, 7-dichloro-2-methyl-1-oxo-1, 2, 3, 4-tetrahydropyrazino[1,2-a]indol-9-yl)carbamate (367 mg, 0.642 mmol) in DMF (5 mL). Add NBS (175 mg, 0.983 mmol). Stir 1 hour at RT. Quench with water, wash with aqueous 4% LiCl, extract with EtOAc, dry over anhydrous sodium sulfate, filter, and concentrate. Purify by flash column chromatography (EtOAc/petroleum ether) to yield tert-butyl (10-bromo-6, 7-dichloro-2-methyl-1-oxo-1, 2, 3, 4-tetrahydropyrazino[1,2-a]indol-9-yl)(2-hydroxyethyl) carbamate (180 mg, 0.315 mmol, 49% Yield). ES-MS (m/z): 450.0/452.0/453.9 (M+H, -tBu).

### Intermediate 223

### tert-Butyl (6,7-dichloro-2-methyl-1-oxo-10-(1-(tetrahydro-2H-pyran-2-yl)-1H-pyrazol-4-yl)-1,2,3,4-tetrahydropyrazino[1,2-a]indol-9-yl)(2-hydroxyethyl)carbamate

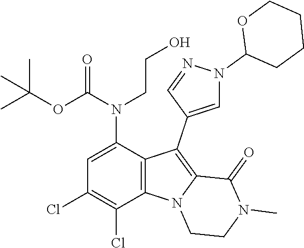 [SMILES: CN1CCn2c(c(-c3cnn(C4CCCCO4)c3)c3c(N(CCO)C(=O)OC(C)(C)C)cc(Cl)c(Cl)c32)C1=O]

Suspend tert-butyl (10-bromo-6, 7-dichloro-2-methyl-1-oxo-1, 2, 3, 4-tetrahydro pyrazino[1,2-a]indol-9-yl)(2-hydroxyethyl) carbamate (110 mg, 0.193 mmol), 1-(tetrahydro-2H-pyran-2-yl)-4-(4,4,5,5-tetramethyl-1,3,2-dioxaborolan-2-yl)-1H-pyrazole (115 mg, 0.393 mmol), Pd(dppf)Cl₂ (120 mg, 0.180 mmol) and K₂CO₃ (290 mg, 2.74 mmol) in 1,4-dioxane (4 mL) and water (1 mL). Sparge with nitrogen and heat to 90° C. for 2 hours. Cool to RT, dilute with EtOAc, wash with brine, dry over sodium sulfate, filter, and concentrate. Purify by flash column chromatography (EtOAc/petroleum ether) to yield tert-butyl (6,7-dichloro-2-methyl-1-oxo-10-(1-(tetrahydro-2H-pyran-2-yl)-1H-pyrazol-4-yl)-1,2,3,4-tetrahydropyrazino[1,2-a]indol-9-yl)(2-hydroxyethyl)carbamate (50 mg, 0.080 mmol, 41% Yield). ES-MS (m/z): 578.2/580.1 (M+H).

### EXAMPLE 87

### 6,7-Dichloro-9-((2-hydroxyethyl)amino)-2-methyl-10-(1H-pyrazol-4-yl)-3,4-dihydropyrazino[1,2-a]indol-1(2H)-one

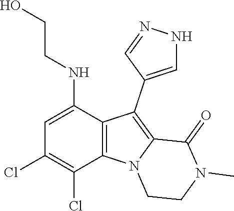 [SMILES: CN1CCn2c(c(-c3cn[nH]c3)c3c(NCCO)cc(Cl)c(Cl)c32)C1=O]

Suspend tert-butyl (6,7-dichloro-2-methyl-1-oxo-10-(1-(tetrahydro-2H-pyran-2-yl)-1H-pyrazol-4-yl)-1, 2, 3, 4-tetrahydropyrazino[1, 2-a]indol-9-yl)(2-hydroxyethyl) carbamate (50 mg, 0.080 mmol) in 6 N aqueous HCl (2 mL) and THF (2 mL) and stir for 1 hour at RT. Concentrate and purify by prep-HPLC to yield 6,7-dichloro-9-((2-hydroxyethyl)amino)-2-methyl-10-(1H-pyrazol-4-yl)-3,4-dihydropyrazino[1,2-a]indol-1(2H)-one (11 mg, 0.027 mmol, 36% Yield). ES-MS (m/z): 394.3/396.3 (M+H). ¹H NMR (400 MHz, DMSO): 7.62 (s, 2H), 6.27 (s, 1H), 4.81 (dd, J=6.4, 5.0 Hz, 2H), 3.72 (dd, J=6.4, 5.0 Hz, 2H), 3.38 (t, J=5.5 Hz, 2H), 3.03 (t, J=5.5 Hz, 2H), 2.93 (s, 3H).

### SCHEME FOR EXAMPLES 88 & 89

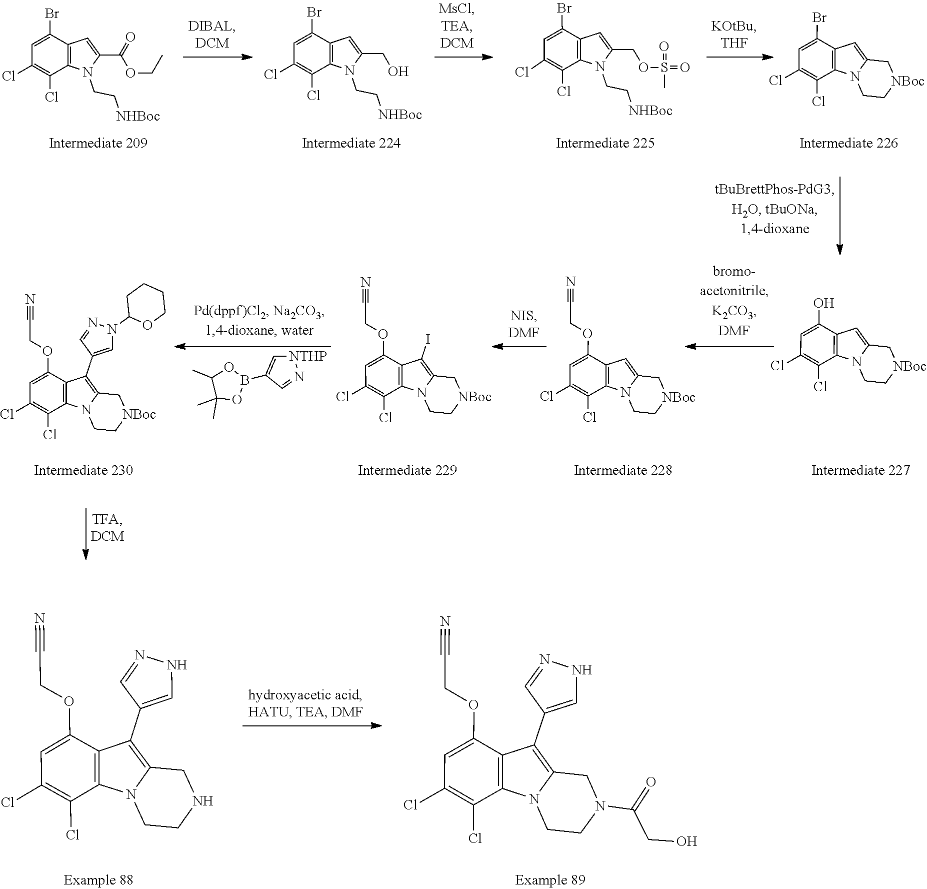

### Intermediate 224

### tert-Butyl (2-(4-bromo-6, 7-dichloro-2-(hydroxymethyl)-1H-indol-1-yl)ethyl)carbamate

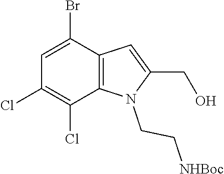 [SMILES: CC(C)(C)OC(=O)NCCn1c(CO)cc2c(Br)cc(Cl)c(Cl)c21]

Dissolve ethyl 4-bromo-1-(2-((tert-butoxycarbonyl)amino)ethyl)-6, 7-dichloro-1H-indole-2-carboxylate (6.00 g, 10.0 mmol) in DCM (120 mL). Add DIBAL (40 mL, 40 mmol, 1M in toluene) at −78° C. under nitrogen. Stir at −78° C. for 1 hour. Quench with saturated aqueous potassium sodium tartrate and stir at RT for 1 hour. Extract with DCM, wash with brine, dry over anhydrous sodium sulfate, filter, and concentrate. Purify by flash column chromatography (EtOAc/petroleum ether) to yield tert-butyl (2-(4-bromo-6, 7-dichloro-2-(hydroxymethyl)-1H-indol-1-yl)ethyl)carbamate (4.00 g, 8.20 mmol, 82% Yield).

### Intermediate 225

### (4-Bromo-1-(2-((tert-butoxycarbonyl)amino)ethyl)-6,7-dichloro-1H-indol-2-yl)methyl methanesulfonate

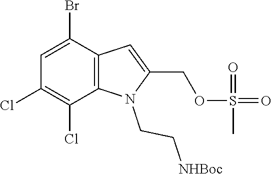 [SMILES: CC(C)(C)OC(=O)NCCn1c(COS(C)(=O)=O)cc2c(Br)cc(Cl)c(Cl)c21]

Dissolve tert-butyl (2-(4-bromo-6, 7-dichloro-2-(hydroxymethyl)-1H-indol-1-yl)ethyl)carbamate and Et₃N (3.6 mL, 26 mmol) in DCM (17 mL). Add MsCl (1.84 g, 15.9 mmol) at 0° C. Stir for 2 hours at RT. Quench with saturated aqueous sodium bicarbonate and stir vigorously for 15 minutes. Dilute with DCM, wash with brine, dry over sodium sulfate, filter, and concentrate. Purify by flash column chromatography (EtOAc/petroleum ether) to yield (4-bromo-1-(2-((tert-butoxycarbonyl)amino)ethyl)-6,7-dichloro-1H-indol-2-yl)methyl methanesulfonate (2.10 g, 3.90 mmol, 47% Yield).

### Intermediate 226

### tert-Butyl 9-bromo-6,7-dichloro-3,4-dihydropyrazino[1,2-a]indole-2 (1H)-carboxylate

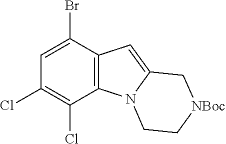 [SMILES: CC(C)(C)OC(=O)N1CCn2c(cc3c(Br)cc(Cl)c(Cl)c32)C1]

Dissolve (4-bromo-1-(2-((tert-butoxycarbonyl)amino)ethyl)-6, 7-dichloro-1H-indol-2-yl)methyl methanesulfonate (2.10 g, 3.90 mmol) in THF (40 mL). Add potassium tert-butoxide (1.20 g, 11.0 mmol). Stir for 2 hours at 60° C. Quench with water and stir vigorously for 15 minutes. Dilute with EtOAc, wash with brine, dry over sodium sulfate, filter, and concentrate. Purify by flash column chromatography (EtOAc/petroleum ether) to yield tert-butyl 9-bromo-6, 7-dichloro-3, 4-dihydropyrazino[1,2-a]indole-2(1H)-carboxylate (1.30 g, 2.94 mmol, 76% Yield).

### Intermediate 227

### tert-Butyl 6,7-dichloro-9-hydroxy-3,4-dihydropyrazino[1,2-a]indole-2(1H)-carboxylate

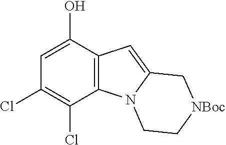 [SMILES: CC(C)(C)OC(=O)N1CCn2c(cc3c(O)cc(Cl)c(Cl)c32)C1]

Dissolve tert-butyl 9-bromo-6, 7-dichloro-3, 4-dihydropyrazino[1,2-a]indole-2(1H)-carboxylate (1.30 g, 3.09 mmol), t-BuONa (766 mg, 7.70 mmol) and t-BuBrettPhos-Pd-G₃ (270 mg, 0.310 mmol) in dioxane (40 mL) and water (4 mL). Sparge with nitrogen and heat to 65° C. for 3 hours. Quench with water and extracted with EtOAc, wash with brine, dry over sodium sulfate, filter, and concentrate. Purify by flash column chromatography (EtOAc/petroleum ether) to yield tert-butyl 6, 7-dichloro-9-hydroxy-3, 4-dihydropyrazino[1,2-a]indole-2(1H)-carboxylate (820 mg, 2.10 mmol, 67% Yield).

### Intermediate 228

### tert-Butyl 6,7-dichloro-9-(cyanomethoxy)-3,4-dihydropyrazino[1,2-a]indole-2(1H)-carboxylate

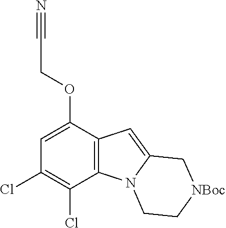 [SMILES: CC(C)(C)OC(=O)N1CCn2c(cc3c(OCC#N)cc(Cl)c(Cl)c32)C1]

Dissolve tert-butyl 6, 7-dichloro-9-hydroxy-3, 4-dihydropyrazino[1,2-a]indole-2(1H)-carboxylate (400 mg, 1.41 mmol) in DMF (10 mL). Add bromoacetonitrile (380 mg, 3.16 mmol) and potassium carbonate (280 mg, 2.02 mmol). Stir for 2 hours at RT. Add water and extract with EtOAc. Wash with brine, dry over anhydrous sodium sulfate, filter, and concentrate. Purify by flash column chromatography (EtOAc/petroleum ether) to yield tert-butyl 6, 7-dichloro-9-(cyano methoxy)-3, 4-dihydropyrazino[1,2-a]indole-2(1H)-carboxylate (340 mg, 0.780 mmol, 78% Yield).

### Intermediate 229

### tert-Butyl 6,7-dichloro-9-(cyanomethoxy)-10-iodo-3,4-dihydropyrazino[1,2-a]indole-2(1H)-carboxylate

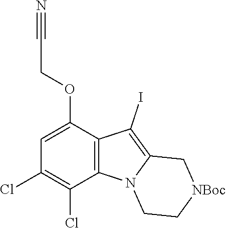 [SMILES: CC(C)(C)OC(=O)N1CCn2c(c(I)c3c(OCC#N)cc(Cl)c(Cl)c32)C1]

Dissolve tert-butyl 6, 7-dichloro-9-(cyanomethoxy)-3, 4-dihydropyrazino[1,2-a]indole-2(1H)-carboxylate (340 mg, 1.16 mmol) in DMF (10 mL). Add NIS (320 mg, 1.40 mmol). Stir for 2 hours at RT. Quench with saturated aqueous Na₂SO₃ and stir vigorously for 15 min. Extract with EtOAc, wash with brine, dry over sodium sulfate, filter, and concentrate. Purify by flash column chromatography (EtOAc/petroleum ether) to yield tert-butyl 6, 7-dichloro-9-(cyanomethoxy) -10-iodo-3, 4-dihydropyrazino[1,2-a]indole-2(1H)-carboxylate (380 mg, 0.71 mmol, 71% Yield).

### Intermediate 230

### tert-Butyl 6,7-dichloro-9-(cyanomethoxy)-10-(1-(tetrahydro-2H-pyran-2-yl)-1H-pyrazol-4-yl)-3,4-dihydropyrazino[1,2-a]indole-2(1H)-carboxylate

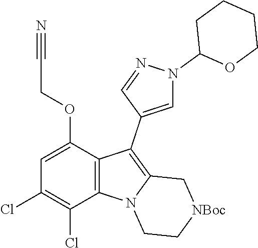 [SMILES: CC(C)(C)OC(=O)N1CCn2c(c(-c3cnn(C4CCCCO4)c3)c3c(OCC#N)cc(Cl)c(Cl)c32)C1]

Dissolve tert-butyl 6, 7-dichloro-9-(cyanomethoxy)-10-iodo-3, 4-dihydropyrazino[1,2-a]indole-2(1H)-carboxylate (320 mg, 0.613 mmol) and 1-(tetrahydro-2H-pyran-2-yl)-4-(4, 4, 5, 5-tetramethyl-1,3,2-dioxaborolan-2-yl)-1H-pyrazole (220 mg, 0.752 mmol) in 1,4-dioxane (4 mL) and water (1 mL). Add Pd(dppf)Cl₂ (48 mg, 0.063 mmol) and sodium carbonate (123 mg, 1.16 mmol). Sparge with nitrogen stir for 2 hours at 90° C. Concentrate and purify by flash column chromatography (EtOAc/petroleum ether) to yield tert-butyl 6,7-dichloro-9-(cyanomethoxy)-10-(1-(tetrahydro-2H-pyran-2-yl)-1H-pyrazol-4-yl)-3,4-dihydropyrazino[1,2-a]indole-2(1H)-carboxylate as a yellow solid (200 mg, 0.322 mmol, 53% Yield).

### EXAMPLE 88

### 2-((6,7-Dichloro-10-(1H-pyrazol-4-yl)-1,2,3,4-tetrahydropyrazino[1,2-a]indol-9-yl)oxy)acetonitrile

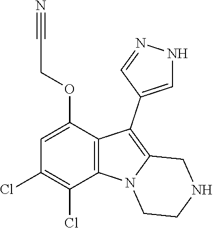 [SMILES: N#CCOc1cc(Cl)c(Cl)c2c1c(-c1cn[nH]c1)c1n2CCNC1]

Dissolve tert-butyl 6, 7-dichloro-9-(cyanomethoxy)-10-(1-(tetrahydro-2H-pyran-2-yl)-1H-pyrazol-4-yl)-3, 4-dihydropyrazino[1,2-a]indole-2(1H)-carboxylate (200 mg, 0.250 mmol) in DCM (4 mL). Add TFA (2 mL) and stir for 1 hour at RT. Concentrate and treat with saturated aqueous NaHCO₃ to pH 8 and extract with EtOAc. Wash with brine, dry over anhydrous sodium sulfate, filter and concentrate to yield the crude product (130 mg). Purify 50 mg of the crude product by prep-HPLC to yield 2-((6,7-dichloro-10-(1H-pyrazol-4-yl)-1,2,3,4-tetrahydropyrazino[1,2-a]indol-9-yl)oxy)acetonitrile (5.8 mg, 0.02 mmol, 4% Yield, formic acid salt). ES-MS (m/z): 362.2/364.3 (M+H). ¹H NMR (400 MHz, DMSO): 8.18 (s, 0.8H, formic acid), 7.60 (br s, 2H), 6.98 (s, 1H), 5.17 (s, 2H), 4.51 (t, J=5.5 Hz, 2H), 3.97 (s, 2H), 3.14 (t, J=5.5 Hz, 2H).

### EXAMPLE 89

### 2-((6,7-Dichloro-2-(2-hydroxyacetyl)-10-(1H-pyrazol-4-yl)-1,2,3,4-tetrahydropyrazino[1,2-a]indol-9-yl)oxy)acetonitrile

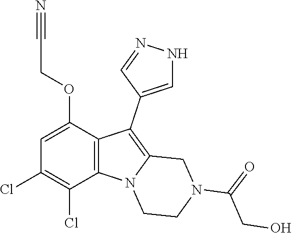 [SMILES: N#CCOc1cc(Cl)c(Cl)c2c1c(-c1cn[nH]c1)c1n2CCN(C(=O)CO)C1]

Dissolve 2-((6, 7-dichloro-10-(1H-pyrazol-4-yl)-1, 2, 3, 4-tetrahydropyrazino[1,2-a]indol-9-yl)oxy) acetonitrile (60 mg, 0.17 mmol) in DMF (2 mL). Add hydroxyacetic acid (18 mg, 0.23 mmol), Et₃N (50 mg, 0.47 mmol) and HATU (73 mg, 0.19 mmol). Stir for 1 hour at RT. Concentrate and purify by prep-HPLC to yield 2-((6,7-dichloro-2-(2-hydroxyacetyl)-10-(1H-pyrazol-4-yl)-1,2,3,4-tetrahydropyrazino[1,2-a]indol-9-yl)oxy) acetonitrile (16.01 mg, 0.040 mmol, 23% Yield). ES-MS (m/z): 420.0/422.0 (M+H). ¹H NMR (400 MHz, DMSO): 12.91 (br s, 1H), 7.90-7.45 (m, 2H), 7.02 (s, 1H), 5.18 (s, 2H), 4.88 (t, J=5.6 Hz, 1H), 4.83-4.62 (m, 4H), 4.24-4.10 (m, 2H), 3.96-3.85 (m, 2H).

### SCHEME FOR EXAMPLES 90 & 91

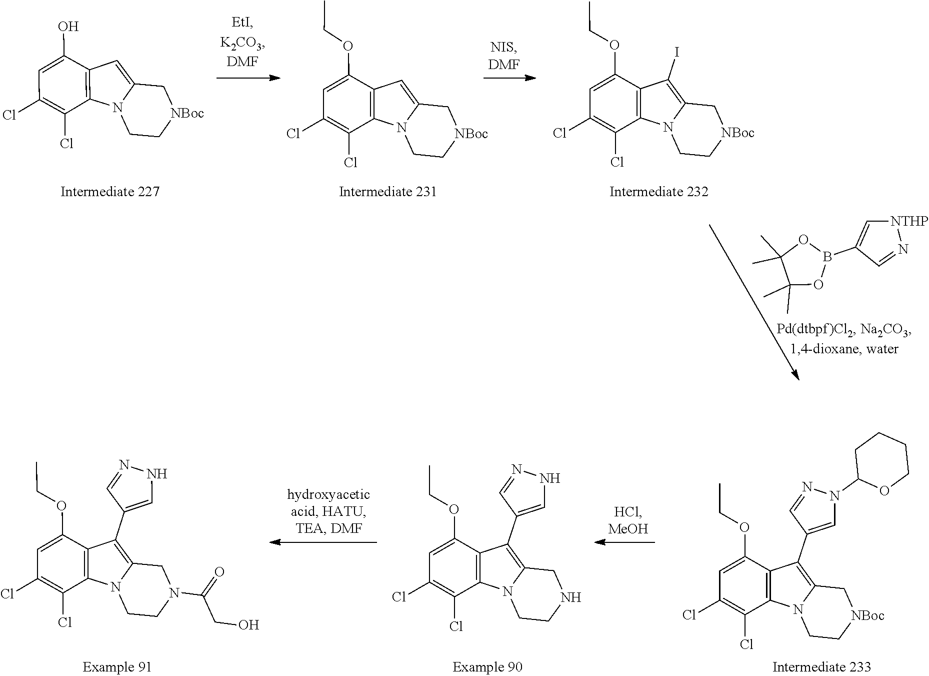

### Intermediate 231

### 3, 4-Dichloro-1-ethoxy-6, 7, 8, 9-tetrahydropyrido[1,2-a]indole

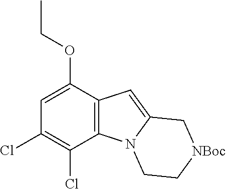 [SMILES: CCOc1cc(Cl)c(Cl)c2c1cc1n2CCN(C(=O)OC(C)(C)C)C1]

Dissolve 3, 4-dichloro-6, 7, 8, 9-tetrahydropyrido[1,2-a]indol-1-ol (280 mg, 0.994 mmol) in DMF (5 mL). Add EtI (235 mg, 1.50 mmol) and K₂CO₃ (180 mg, 1.30 mmol) and stir for 2 hours at RT. Dilute with water, extract with EtOAc, wash with brine, dry over anhydrous Na₂SO₄, filter, and concentrate. Purify by flash column chromatography (EtOAc/petroleum ether) to yield 3, 4-dichloro-1-ethoxy-6, 7, 8, 9-tetrahydropyrido[1,2-a]indole (217 mg, 0.748 mmol, 75% Yield).

### Intermediate 232

### tert-Butyl 6,7-dichloro-9-ethoxy-10-iodo-3, 4-dihydro-1H-pyrazino[1,2-a]indole-2-carboxylate

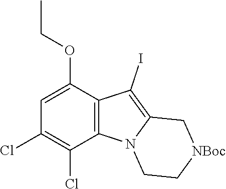 [SMILES: CCOc1cc(Cl)c(Cl)c2c1c(I)c1n2CCN(C(=O)OC(C)(C)C)C1]

Dissolve tert-butyl 6, 7-dichloro-9-ethoxy-3,4-dihydro-1H-pyrazino[1,2-a]indole-2-carboxylate (217 mg, 0.551 mmol) in DMF (5 mL) and add NIS (195 mg, 0.849 mmol). Stir for 1 hour at RT. Quench with saturated aqueous Na₂SO₃ and extract with EtOAc. Wash with brine, dry over anhydrous Na₂SO₄, filter, and concentrate to yield tert-butyl 6, 7-dichloro-9-ethoxy-10-iodo-3, 4-dihydro-1H-pyrazino[1,2-a]indole-2-carboxylate (250 mg, 0.391 mmol, 71% Yield).

### Intermediate 233

### tert-Butyl 6, 7-dichloro-9-ethoxy-10-(1-tetrahydropyran-2-ylpyrazol-4-yl)-3, 4-dihydro-1H-pyrazino[1,2-a]indole-2-carboxylate

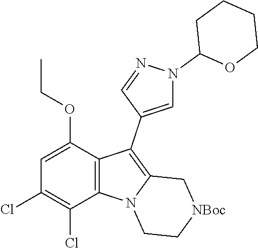 [SMILES: CCOc1cc(Cl)c(Cl)c2c1c(-c1cnn(C3CCCCO3)c1)c1n2CCN(C(=O)OC(C)(C)C)C1]

Suspend tert-butyl 6, 7-dichloro-9-ethoxy-10-iodo-3, 4-dihydro-1H-pyrazino[1,2-a]indole-2-carboxylate (250 mg, 0.391 mmol), 1-(tetrahydro-2h-pyran-2-yl)-4-(4,4,5,5-tetramethyl-1,3,2-dioxaborolan-2-yl)-1h-pyrazole (175 mg, 0.597 mmol), Pd(dtbpf)Cl₂ (30 mg, 0.045 mmol) and Na₂CO₃ (125 mg, 1.17 mmol) in 1,4-dioxane (4 mL) and water (1 mL). Sparge with nitrogen and heat to 90° C. for 1 hour. Cool to RT, dilute with EtOAc, wash with brine, dry over anhydrous Na₂SO₄, filter and concentrate. Purify by flash column chromatography (EtOAc/petroleum ether), then further purify by prep-HPLC to yield tert-butyl 6, 7-dichloro-9-ethoxy-10-(1-tetrahydropyran-2-ylpyrazol-4-yl)-3, 4-dihydro-1H-pyrazino[1, 2-a]indole-2-carboxylate (95 mg, 0.17 mmol, 44% Yield) as an off-white solid. ES-MS (m/z): 535.2/537.2 (M+H).

### EXAMPLE 90

### 6, 7-Dichloro-9-ethoxy-10-(1H-pyrazol-4-yl)-1, 2, 3, 4-tetrahydropyrazino[1,2-a]indole

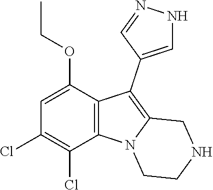 [SMILES: CCOc1cc(Cl)c(Cl)c2c1c(-c1cn[nH]c1)c1n2CCNC1]

Dissolve tert-butyl 6, 7-dichloro-9-ethoxy-10-(1-tetrahydropyran-2-ylpyrazol-4-yl)-3,4-dihydro-1H-pyrazino[1,2-a]indole-2-carboxylate (40 mg, 0.070 mmol) in 4N HCl/MeOH (2 mL). Stir 1 hour at RT. Concentrate to yield 6, 7-dichloro-9-ethoxy-10-(1H-pyrazol-4-yl)-1, 2, 3, 4-tetrahydro pyrazino[1,2-a]indole (28 mg, 0.070 mmol, 97% Yield, HCl salt). ES-MS (m/z): 351.0/353.0 (M+H). ¹H NMR (400 MHz, DMSO): 9.84 (br s, 2H), 7.75 (s, 2H), 6.83 (s, 1H), 4.82 (t, J=5.7 Hz, 2H), 4.44 (s, 2H), 4.06 (q, J=6.9 Hz, 2H), 3.67-3.58 (m, 2H), 1.25 (t, J=6.9 Hz, 3H).

### EXAMPLE 91

### 1-[6,7-Dichloro-9-ethoxy-10-(1H-pyrazol-4-yl)-3,4-dihydro-1H-pyrazino[1,2-a]indol-2-yl]-2-hydroxy-ethanone

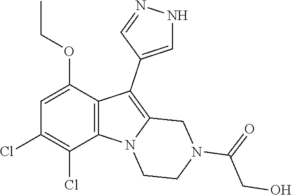 [SMILES: CCOc1cc(Cl)c(Cl)c2c1c(-c1cn[nH]c1)c1n2CCN(C(=O)CO)C1]

Dissolve 6, 7-dichloro-9-ethoxy-10-(1H-pyrazol-4-yl)-1, 2, 3, 4-tetrahydropyrazino[1,2-a]indole (30 mg, 0.070 mmol, HCl salt) in DMF (2 mL). Add hydroxyacetic acid (10 mg, 0.13 mmol), Et₃N (38 mg, 0.37 mmol) and HATU (36 mg, 0.10 mmol). Stir for 16 hours at RT. Concentrate and purify by prep-HPLC to yield 1-[6,7-dichloro-9-ethoxy-10-(1H-pyrazol-4-yl)-3,4-dihydro-1H-pyrazino[1,2-a]indol-2-yl]-2-hydroxy-ethanone (16 mg, 0.040 mmol, 52% Yield). ES-MS (m/z): 409.0/411.0 (M+H). ¹H NMR (400 MHz, DMSO): 7.74-7.62 (m, 2H), 6.77 (s, 1H), 4.82-4.74 (m, 2H), 4.74-4.60 (m, 2H), 4.20 (br s, 1.5H, major rotamer), 4.13 (br s, 0.5H, minor rotamer), 4.04 (q, J=6.9 Hz, 2H), 3.96-3.83 (m, 2H), 1.24 (t, J=6.9 Hz, 3H).

### SCHEME FOR EXAMPLE 92

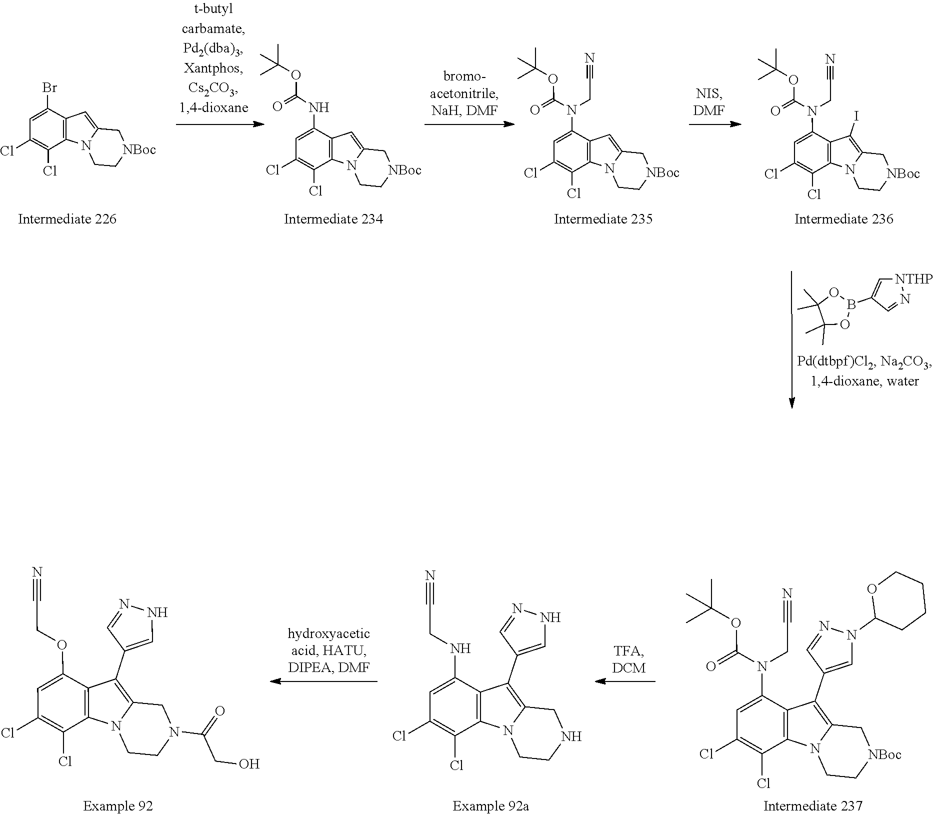

### Intermediate 234

### tert-Butyl 9-(tert-butoxycarbonylamino)-6, 7-dichloro-3,4-dihydro-1H-pyrazino[1,2-a]indole-2-carboxylate

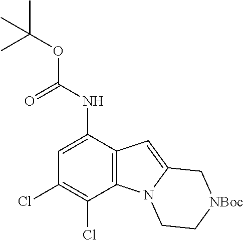 [SMILES: CC(C)(C)OC(=O)Nc1cc(Cl)c(Cl)c2c1cc1n2CCN(C(=O)OC(C)(C)C)C1]

Suspend tert-butyl 9-bromo-6, 7-dichloro-3, 4-dihydro-1H-pyrazino[1,2-a]indole-2-carboxylate (500 mg, 1.13 mmol), tert-butyl carbamate (293 mg, 2.50 mmol), Pd₂(dba)₃ (105 mg, 0.114 mmol), XantPhos (133 mg, 0.229 mmol) and Cs₂CO₃ (925 mg, 2.83 mmol) in 1,4-dioxane (8 mL). Sparge with nitrogen and heat to 100° C. overnight. Cool to RT and concentrate. Purify by flash column chromatography (EtOAc/hexane) to yield tert-butyl 9-(tert-butoxycarbonylamino)-6,7-dichloro-3,4-dihydro-1H-pyrazino[1,2-a]indole-2-carboxylate (385 mg, 0.801 mmol, 71% Yield).

### Intermediate 235

### tert-Butyl 9-[tert-butoxycarbonyl(cyanomethyl)amino]-6,7-dichloro-3,4-dihydro-1H-pyrazino[1,2-a]indole-2-carboxylate

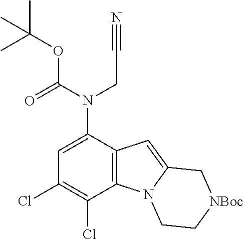 [SMILES: CC(C)(C)OC(=O)N1CCn2c(cc3c(N(CC#N)C(=O)OC(C)(C)C)cc(Cl)c(Cl)c32)C1]

Dissolve tert-butyl 9-(tert-butoxycarbonylamino)-6, 7-dichloro-3, 4-dihydro-1H-pyrazino[1,2-a]indole-2-carboxylate (385 mg, 0.801 mmol) in DMF (6 mL). Add NaH (50 mg, 1.3 mmol, 60% dispersion in mineral oil) and stir for 0.5 hour at RT. Add bromoacetonitrile (150 mg, 1.25 mmol) and stir for 5 hours at RT. Quench with H₂O at 0° C. and extract with EtOAc. Wash with brine, dry over anhydrous Na₂SO₄, filter and concentrate. Purify by flash column chromatography (EtOAc/petroleum ether) to yield tert-butyl 9-[tert-butoxycarbonyl(cyanomethyl)amino]-6, 7-dichloro-3, 4-dihydro-1H-pyrazino[1,2-a]indole-2-carboxylate (200 mg, 0.383 mmol, 48% Yield) as a light yellow solid. ES-MS (m/z): 383.0/384.9 (M+H, −2 tBu).

### Intermediate 236

### tert-Butyl 9-[tert-butoxycarbonyl(cyanomethyl)amino]-6,7-dichloro-10-iodo-3,4-dihydro-1H-pyrazino[1,2-a]indole-2-carboxylate

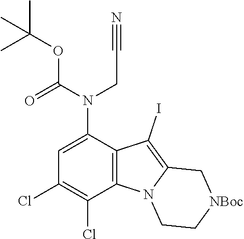 [SMILES: CC(C)(C)OC(=O)N1CCn2c(c(I)c3c(N(CC#N)C(=O)OC(C)(C)C)cc(Cl)c(Cl)c32)C1]

Dissolve tert-butyl 9-[tert-butoxycarbonyl(cyanomethyl)amino]-6, 7-dichloro-3, 4-dihydro-1H-pyrazino[1,2-a]indole-2-carboxylate (235 mg, 0.464 mmol) in DMF (5 mL) and add NIS (165 mg, 0.711 mmol). Stir for 1 hour at RT. Quench with saturated aqueous Na₂SO₃ and extract with EtOAc, wash with brine, dry over anhydrous Na₂SO₄, filter and concentrate to yield tert-butyl 9-[tert-butoxycarbonyl(cyanomethyl)amino]-6, 7-dichloro-10-iodo-3, 4-dihydro-1H-pyrazino[1,2-a]indole-2-carboxylate (246 mg, 0.388 mmol, 83% Yield) as a light yellow solid. ES-MS (m/z): 508.8/510.7 (M+H, −2 tBu).

### Intermediate 237

### tert-Butyl 9-[tert-butoxycarbonyl(cyanomethyl)amino]-6,7-dichloro-10-(1-tetrahydropyran-2-ylpyrazol-4-yl)-3,4-dihydro-1H-pyrazino[1,2-a]indole-2-carboxylate

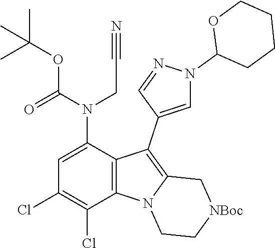 [SMILES: CC(C)(C)OC(=O)N1CCn2c(c(-c3cnn(C4CCCCO4)c3)c3c(N(CC#N)C(=O)OC(C)(C)C)cc(Cl)c(Cl)c32)C1]

Suspend tert-butyl 9-[tert-butoxycarbonyl(cyanomethyl)amino]-6,7-dichloro-10-iodo-3,4-dihydro -1H-pyrazino[1,2-a]indole-2-carboxylate (246 mg, 0.388 mmol), 1-(tetrahydro-2h-pyran-2-yl)-4-(4,4,5,5-tetramethyl-1,3,2-dioxaborolan-2-yl)-1h-pyrazole (175 mg, 0.597 mmol), Pd(dtbpf)Cl₂ (30 mg, 0.045 mmol) and Na₂CO₃ (125 mg, 1.17 mmol) in 1,4-dioxane (4 mL) and water (1 mL). Sparge with nitrogen and heat to 90° C. for 1 hour. Cool to RT, extract with EtOAc, wash with brine, dry over anhydrous Na₂SO₄, filter and concentrate. Purify by flash column chromatography (EtOAc/petroleum ether) to yield tert-butyl 9-[tert-butoxycarbonyl (cyanomethyl)amino]-6,7-dichloro-10-(1-tetrahydropyran-2-ylpyrazol-4-yl)-3,4-dihydro-1H-pyrazino indole-2-carboxylate (90 mg, 0.13 mmol, 34% Yield) as brown oil.

### EXAMPLE 92A

### 2-[[6, 7-Dichloro-10-(1H-pyrazol-4-yl)-1,2,3,4-tetrahydropyrazino[1,2-a]indol-9-yl]amino]acetonitrile

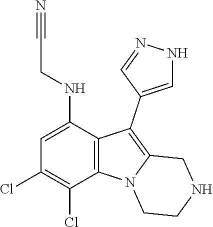 [SMILES: N#CCNc1cc(Cl)c(Cl)c2c1c(-c1cn[nH]c1)c1n2CCNC1]

Suspend tert-butyl 9-[tert-butoxycarbonyl(cyanomethyl)amino]-6, 7-dichloro-10-(1-tetrahydropyran-2-ylpyrazol-4-yl)-3, 4-dihydro-1H-pyrazino[1,2-a]indole-2-carboxylate (90 mg, 0.13 mmol) in DCM (2 mL). Add TFA (2 mL) and stir for 3 hours at RT. Dilute with water, adjust to pH 8 with 1N aqueous NaOH, extract with EtOAc, wash with brine (3×), dry over anhydrous Na₂SO₄, filter, and concentrate to yield 2-[[6, 7-dichloro-10-(1H-pyrazol-4-yl)-1, 2, 3, 4-tetrahydropyrazino[1,2-a]indol-9-yl]amino]acetonitrile (32 mg, 0.044 mmol, 33% Yield). ES-MS (m/z): 361.0/362.7 (M+H).

### EXAMPLE 92

### 2-[[6, 7-Dichloro-2-(2-hydroxyacetyl)-10-(1H-pyrazol-4-yl)-3,4-dihydro-1H-pyrazino[1,2-a]indol-9-yl]amino]acetonitrile

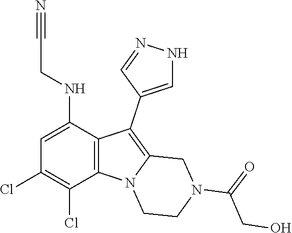 [SMILES: N#CCNc1cc(Cl)c(Cl)c2c1c(-c1cn[nH]c1)c1n2CCN(C(=O)CO)C1]

Suspend 2-[[6, 7-dichloro-10-(1H-pyrazol-4-yl)-1, 2, 3, 4-tetrahydropyrazino[1,2-a]indol-9-yl]amino]acetonitrile (32 mg, 0.044 mmol), hydroxyacetic acid (7 mg, 0.09 mmol), HATU (38 mg, 0.098 mmol) and DIPEA (32 mg, 0.25 mmol) in DMF (2 mL) and stir 1 hour at RT. Dilute with water, extract with EtOAc, wash with brine, dry over anhydrous Na₂SO₄, filter and concentrate. Purify by prep-HPLC to yield 2-[[6, 7-dichloro-2-(2-hydroxyacetyl)-10-(1H-pyrazol-4-yl)-3, 4-dihydro-1H-pyrazino[1,2-a]indol-9-yl]amino]acetonitrile (3.4 mg, 0.0077 mmol, 17% Yield). ES-MS (m/z): 419.2/421.3 (M+H). ¹H NMR (400 MHz, DMSO): 13.12 (br s, 1H), 7.98-7.46 (m, 2H), 6.56 (s, 1H), 5.05-4.93 (m, 1H), 4.92-4.81 (m, 1H), 4.76-4.51 (m, 4H), 4.30 (d, J=6.5 Hz, 2H), 4.24-4.04 (m, 2H), 3.95-3.81 (m, 2H).

### SCHEME FOR EXAMPLE 93

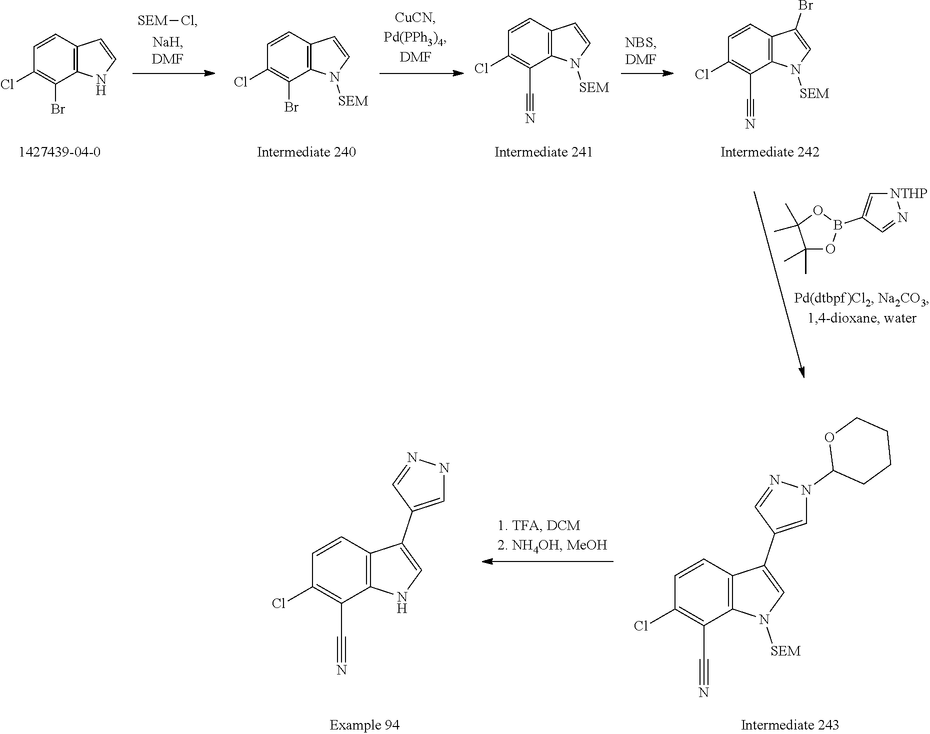

### Intermediate 239

### 2-[(7-Bromo-6-chloro-indol-1-yl)methoxy]ethyl-trimethyl-silane

Dissolve 7-bromo-6-chloro-1H-indole (3.52 g, 9.93 mmol, 65 mass %) in DMF (30 mL). Add sodium hydride (600 mg, 15.0 mmol, 60% dispersion in oil) slowly at 0° C. Stir at 0° C. for 30 min. Add 2-(trimethylsilyl)ethoxymethyl chloride (2.1 g, 12 mmol) and stir at 0° C. for 1 hour. Quench with water and extract with EtOAc. Wash with brine, dry over anhydrous sodium sulfate, filter, and concentrate. Purify by flash chromatography (EtOAc/petroleum ether) to yield 2-[(7-bromo-6-chloro-indol-1-yl)methoxy]ethyl-trimethyl-silane (3.04 g, 8.00 mmol, 80% Yield).

### Intermediate 240

### 6-Chloro-1-(2-trimethylsilylethoxymethyl)indole-7-carbonitrile

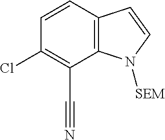 [SMILES: C[Si](C)(C)CCOCn1ccc2ccc(Cl)c(C#N)c21]

Dissolve 2-[(7-bromo-6-chloro-indol-1-yl)methoxy]ethyl-trimethyl-silane (1.5 g, 3.9 mmol), Copper(I) cyanide (0.9 g, 10 mmol) and Pd(PPh₃)₄ (0.46 g, 0.40 mmol, 100 mass %) in DMF (12 mL). Sparge with nitrogen and heat in the microwave at 175° C. for 1 hour. Combine with an additional reaction mixture run at the same scale. Dilute with water and extract with EtOAc (3×). Wash with brine, dry over anhydrous sodium sulfate, filter, and concentrate. Purify by flash chromatography to yield 6-chloro-1-(2-trimethylsilylethoxymethyl)indole-7-carbonitrile (1.71 g, 5.57 mmol, 70% yield for combined reactions).

### Intermediate 241

### 3-Bromo-6-chloro-1-(2-trimethylsilylethoxymethyl)indole-7-carbonitrile

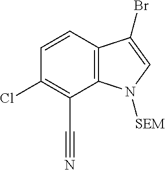 [SMILES: C[Si](C)(C)CCOCn1cc(Br)c2ccc(Cl)c(C#N)c21]

Dissolve 6-chloro-1-(2-trimethylsilylethoxymethyl)indole-7-carbonitrile (700 mg, 2.28 mmol) in DMSO (5 mL, 99.5 mass %) and add NBS (625 mg, 3.44130 mmol, 98 mass %). Stir for 1 hour. Dilute with water and extract with EtOAc (3×). Wash with brine, dry over anhydrous sodium sulfate, filter, and concentrate. Purify by flash chromatography (EtOAc/petroleum ether) to yield 3-bromo-6-chloro-1-(2-trimethylsilylethoxymethyl)indole-7-carbonitrile (738 mg, 1.91 mmol, 84% Yield).

### Intermediate 242

### 6-Chloro-3-(1-tetrahydropyran-2-ylpyrazol-4-yl)-1-(2-trimethylsilylethoxymethyl)indole-7-carbonitrile

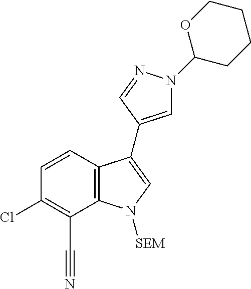 [SMILES: C[Si](C)(C)CCOCn1cc(-c2cnn(C3CCCCO3)c2)c2ccc(Cl)c(C#N)c21]

Dissolve 3-bromo-6-chloro-1-(2-trimethylsilylethoxymethyl)indole-7-carbonitrile (300 mg, 0.778 mmol), 1-(tetrahydro-2H-pyran-2-yl)-4-(4,4,5,5-tetramethyl-1,3,2-dioxaborolan-2-yl)-1H-pyrazole (460 mg, 1.57 mmol), Na₂CO₃ (250 mg, 2.36 mmol) and Pd(dtbpf)Cl₂ (52 mg, 0.078 mmol) in 1,4-dioxane (4 mL) and water (1 mL). Stir at 90° C. for 2 hours under nitrogen. Dilute with water and extract with EtOAc (3×). Wash with brine, dry over anhydrous sodium sulfate, filter, and concentrate. Purify by flash chromatography to yield 6-chloro-3-(1-tetrahydropyran-2-ylpyrazol-4-yl)-1-(2-trimethylsilylethoxymethyl)indole-7-carbonitrile (255 mg, 0.502 mmol, 65% Yield).

### EXAMPLE 93

### 6-Chloro-3-(1H-pyrazol-4-yl)-1H-indole-7-carbonitrile

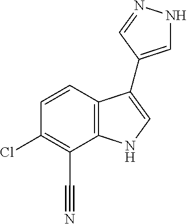 [SMILES: N#Cc1c(Cl)ccc2c(-c3cn[nH]c3)c[nH]c12]

Dissolve 6-chloro-3-(1-tetrahydropyran-2-ylpyrazol-4-yl)-1-(2-trimethylsilylethoxymethyl)indole-7-carbonitrile (200 mg, 0.394 mmol) in TFA (1 mL) and DCM (3 mL, 46.80 mmol). Stir for 1 hour. Concentrate and add aqueous ammonia (28%) and methanol (3 mL). Stir for 30 minutes. Concentrate and purify by prep-HPLC to yield 6-chloro-3-(1H-pyrazol-4-yl)-1H-indole-7-carbonitrile (43 mg, 0.18 mmol, 45% Yield). ES-MS (m/z): 243.2/245.2 (M+H). ¹H NMR (400 MHz, DMSO): 12.92 (br s, 1H), 12.21 (s, 1H), 8.17 (br s, 1H), 8.17 (d, J=8.5 Hz, 1H), 7.91 (br s, 1H), 7.77 (d, J=2.0 Hz, 1H), 7.32 (d, J=8.5 Hz, 1H).

### SCHEME FOR EXAMPLE 94

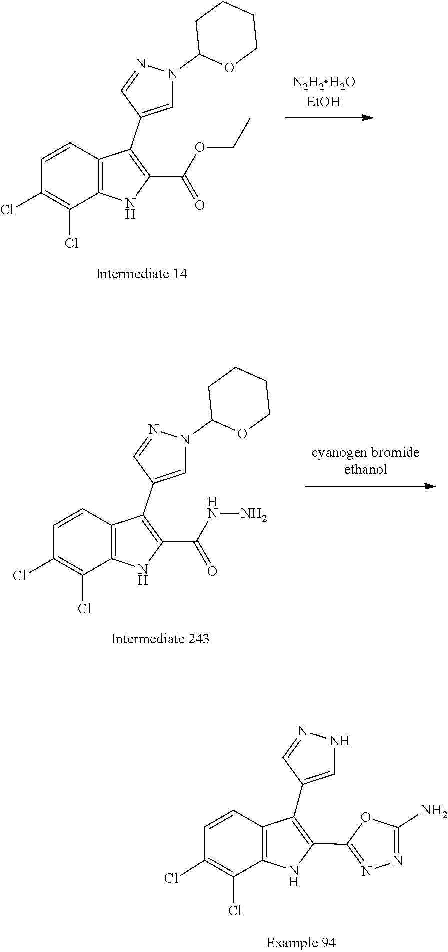 [SMILES: CCOC(=O)c1[nH]c2c(Cl)c(Cl)ccc2c1-c1cnn(C2CCCCO2)c1 ; NNC(=O)c1[nH]c2c(Cl)c(Cl)ccc2c1-c1cnn(C2CCCCO2)c1 ; Nc1nnc(-c2[nH]c3c(Cl)c(Cl)ccc3c2-c2cn[nH]c2)o1]

### Intermediate 243

### 6,7-Dichloro-3-(1-tetrahydropyran-2-ylpyrazol-4-yl)-1H-indole-2-carbohydrazide

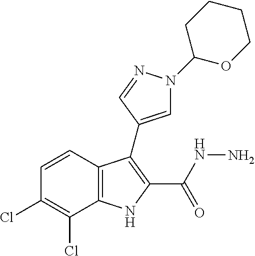 [SMILES: NNC(=O)c1[nH]c2c(Cl)c(Cl)ccc2c1-c1cnn(C2CCCCO2)c1]

Mix ethyl 6,7-dichloro-3-(1-tetrahydropyran-2-ylpyrazol-4-yl)-1H-indole-2-carboxylate (500 mg, 1.23 mmol) with hydrazine monohydrate (0.60 mL, 12.37 mmol) in ethanol (10 mL) in a microwave tube. Heat to 140° C. for 45 min. Stir overnight at RT. Collect precipitate by suction filtration to yield 6,7-dichloro-3-(1-tetrahydropyran-2-ylpyrazol-4-yl)-1H-indole-2-carbohydrazide (400 mg, 1.01 mmol, 83% Yield). ES-MS (m/z): 393.8/395.8 (M+H).

### EXAMPLE 94

### 5-[6,7-Dichloro-3-(1H-pyrazol-4-yl)-1H-indol-2-yl]-1,3,4-oxadiazol-2-amine

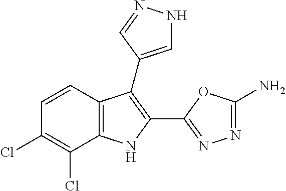 [SMILES: Nc1nnc(-c2[nH]c3c(Cl)c(Cl)ccc3c2-c2cn[nH]c2)o1]

Mix 6,7-dichloro-3-(1-tetrahydropyran-2-ylpyrazol-4-yl)-1H-indole-2-carbohydrazide (370 mg, 0.938 mmol) with cyanogen bromide (110 mg, 1.038 mmol) in ethanol (15 mL) and reflux for 30 minutes. Concentrate, dilute with saturated sodium bicarbonate and extract with an EtOAc/THF mixture. Dry over anhydrous sodium sulfate, filter, and concentrate. Purify by prep-HPLC to yield 5-[6,7-dichloro-3-(1H-pyrazol-4-yl)-1H-indol-2-yl]-1,3,4-oxadiazol-2-amine (101 mg, 0.301 mmol, 32% Yield). ES-MS (m/z): 334.8/336.8 (M+H). ¹H NMR (400 MHz, DMSO): 13.06 (s, 1H), 12.24 (s, 1H), 8.16 (s, 1H), 7.89 (s, 1H), 7.71 (d, J=8.4 Hz, 1H), 7.31 (d, J=8.8 Hz, 2H).

Compounds 1-94 are listed in Table 3 below.

TABLE 3

| Ex. No. | Compound |
|---|---|
| 1 | 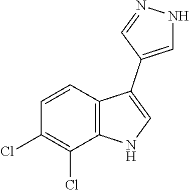 [SMILES: Clc1ccc2c(-c3cn[nH]c3)c[nH]c2c1Cl] |
| 2 | 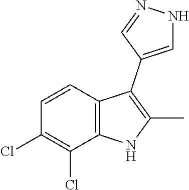 [SMILES: Cc1[nH]c2c(Cl)c(Cl)ccc2c1-c1cn[nH]c1] |
| 3 | 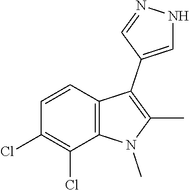 [SMILES: Cc1c(-c2cn[nH]c2)c2ccc(Cl)c(Cl)c2n1C] |
| 4 | 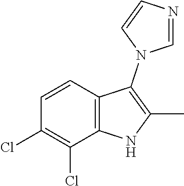 [SMILES: Cc1[nH]c2c(Cl)c(Cl)ccc2c1-n1ccnc1] |
| 5 | 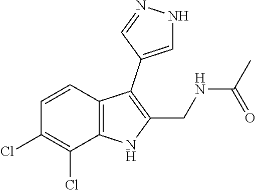 [SMILES: CC(=O)NCc1[nH]c2c(Cl)c(Cl)ccc2c1-c1cn[nH]c1] |
| 6 | 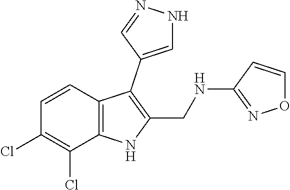 [SMILES: Clc1ccc2c(-c3cn[nH]c3)c(CNc3ccon3)[nH]c2c1Cl] |
| 7 | 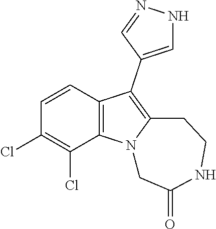 [SMILES: O=C1Cn2c(c(-c3cn[nH]c3)c3ccc(Cl)c(Cl)c32)CCN1] |
| 8 | 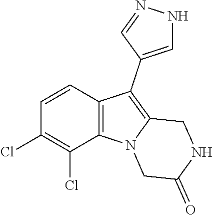 [SMILES: O=C1Cn2c(c(-c3cn[nH]c3)c3ccc(Cl)c(Cl)c32)CN1] |
| 10 | 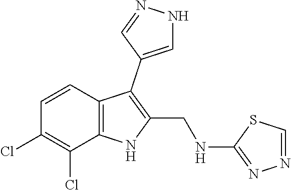 [SMILES: Clc1ccc2c(-c3cn[nH]c3)c(CNc3nncs3)[nH]c2c1Cl] |
| 11 | 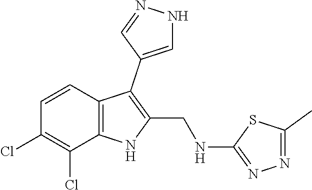 [SMILES: Cc1nnc(NCc2[nH]c3c(Cl)c(Cl)ccc3c2-c2cn[nH]c2)s1] |
| 12 | 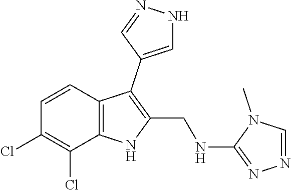 [SMILES: Cn1cnnc1NCc1[nH]c2c(Cl)c(Cl)ccc2c1-c1cn[nH]c1] |
| 13 | 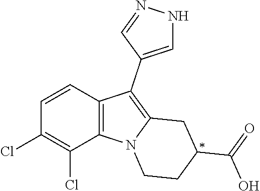 [SMILES: O=C(O)C1CCn2c(c(-c3cn[nH]c3)c3ccc(Cl)c(Cl)c32)C1] Isomer 1 |
| 14 | 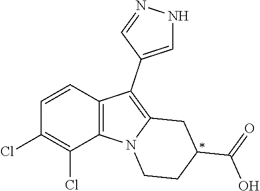 [SMILES: O=C(O)C1CCn2c(c(-c3cn[nH]c3)c3ccc(Cl)c(Cl)c32)C1] Isomer 2 |
| 15 | 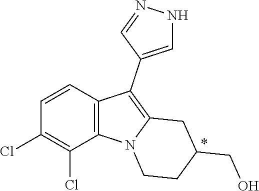 [SMILES: OCC1CCn2c(c(-c3cn[nH]c3)c3ccc(Cl)c(Cl)c32)C1] Isomer 1 |
| 16 | 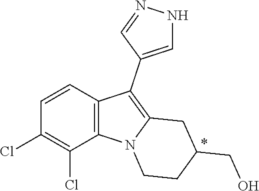 [SMILES: OCC1CCn2c(c(-c3cn[nH]c3)c3ccc(Cl)c(Cl)c32)C1] Isomer 2 |
| 17 | 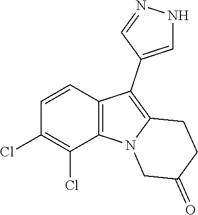 [SMILES: O=C1CCc2c(-c3cn[nH]c3)c3ccc(Cl)c(Cl)c3n2C1] |
| 18 | 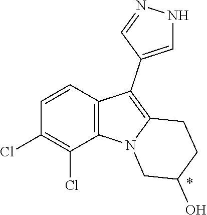 [SMILES: OC1CCc2c(-c3cn[nH]c3)c3ccc(Cl)c(Cl)c3n2C1] Isomer 1 |
| 19 | 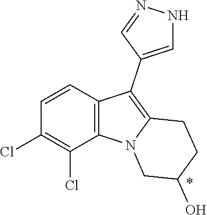 [SMILES: OC1CCc2c(-c3cn[nH]c3)c3ccc(Cl)c(Cl)c3n2C1] Isomer 2 |
| 20 | 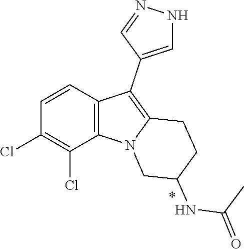 [SMILES: CC(=O)NC1CCc2c(-c3cn[nH]c3)c3ccc(Cl)c(Cl)c3n2C1] Isomer 1 |
| 21 | 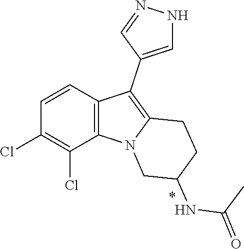 [SMILES: CC(=O)NC1CCc2c(-c3cn[nH]c3)c3ccc(Cl)c(Cl)c3n2C1] Isomer 2 |
| 22 | 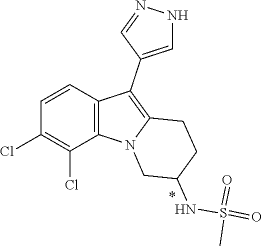 [SMILES: CS(=O)(=O)NC1CCc2c(-c3cn[nH]c3)c3ccc(Cl)c(Cl)c3n2C1] Isomer 1 |
| 23 | 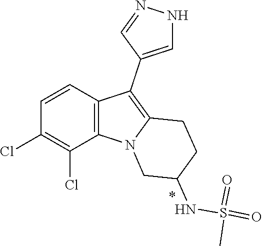 [SMILES: CS(=O)(=O)NC1CCc2c(-c3cn[nH]c3)c3ccc(Cl)c(Cl)c3n2C1] Isomer 2 |
| 24 | 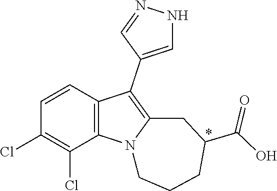 [SMILES: O=C(O)C1CCCn2c(c(-c3cn[nH]c3)c3ccc(Cl)c(Cl)c32)C1] Isomer 1 |
| 25 | 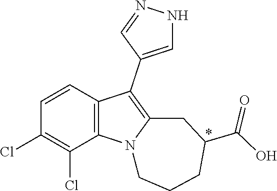 [SMILES: O=C(O)C1CCCn2c(c(-c3cn[nH]c3)c3ccc(Cl)c(Cl)c32)C1] Isomer 2 |
| 26 | 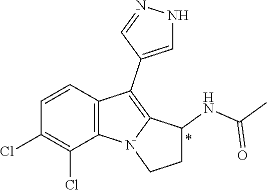 [SMILES: CC(=O)NC1CCn2c1c(-c1cn[nH]c1)c1ccc(Cl)c(Cl)c12] Isomer 1 |
| 27 | 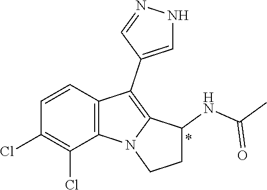 [SMILES: CC(=O)NC1CCn2c1c(-c1cn[nH]c1)c1ccc(Cl)c(Cl)c12] Isomer 2 |
| 28 | 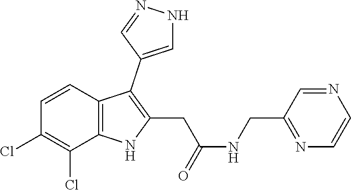 [SMILES: O=C(Cc1[nH]c2c(Cl)c(Cl)ccc2c1-c1cn[nH]c1)NCc1cnccn1] |
| 29 | 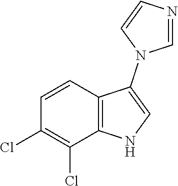 [SMILES: Clc1ccc2c(-n3ccnc3)c[nH]c2c1Cl] |
| 30 | 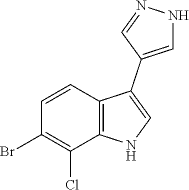 [SMILES: Clc1c(Br)ccc2c(-c3cn[nH]c3)c[nH]c12] |
| 31 | 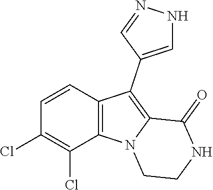 [SMILES: O=C1NCCn2c1c(-c1cn[nH]c1)c1ccc(Cl)c(Cl)c12] |
| 32 | 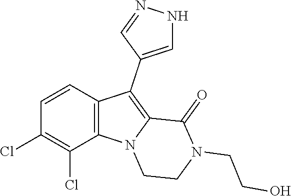 [SMILES: O=C1c2c(-c3cn[nH]c3)c3ccc(Cl)c(Cl)c3n2CCN1CCO] |
| 33 | 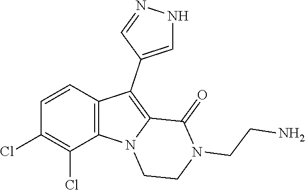 [SMILES: NCCN1CCn2c(c(-c3cn[nH]c3)c3ccc(Cl)c(Cl)c32)C1=O] |
| 34 | 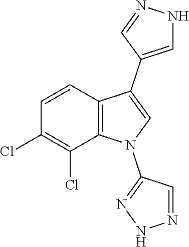 [SMILES: Clc1ccc2c(-c3cn[nH]c3)cn(-c3cn[nH]n3)c2c1Cl] |
| 35 | 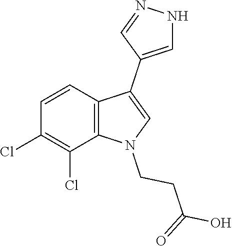 [SMILES: O=C(O)CCn1cc(-c2cn[nH]c2)c2ccc(Cl)c(Cl)c21] |
| 36 | 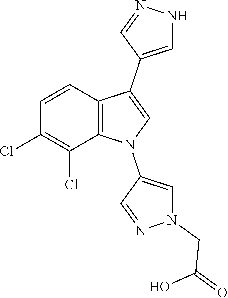 [SMILES: O=C(O)Cn1cc(-n2cc(-c3cn[nH]c3)c3ccc(Cl)c(Cl)c32)cn1] |
| 37 | 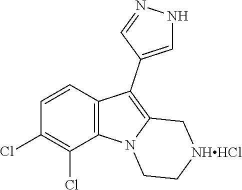 |
| 38 | 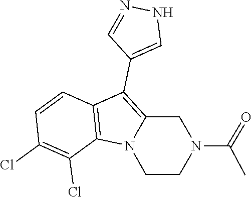 [SMILES: CC(=O)N1CCn2c(c(-c3cn[nH]c3)c3ccc(Cl)c(Cl)c32)C1] |
| 39 | 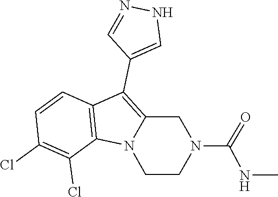 [SMILES: CNC(=O)N1CCn2c(c(-c3cn[nH]c3)c3ccc(Cl)c(Cl)c32)C1] |
| 40 | 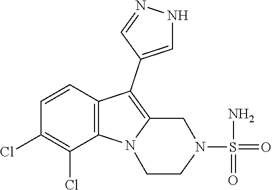 [SMILES: NS(=O)(=O)N1CCn2c(c(-c3cn[nH]c3)c3ccc(Cl)c(Cl)c32)C1] |
| 41 | 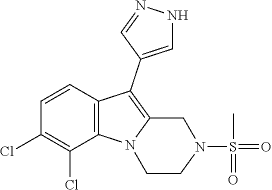 [SMILES: CS(=O)(=O)N1CCn2c(c(-c3cn[nH]c3)c3ccc(Cl)c(Cl)c32)C1] |
| 42 | 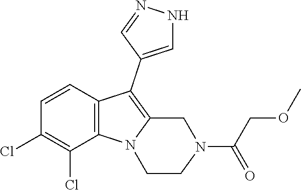 [SMILES: COCC(=O)N1CCn2c(c(-c3cn[nH]c3)c3ccc(Cl)c(Cl)c32)C1] |
| 43 | 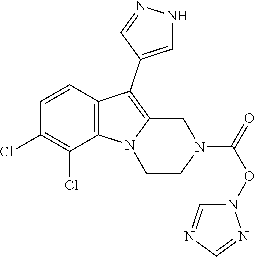 [SMILES: O=C(On1cncn1)N1CCn2c(c(-c3cn[nH]c3)c3ccc(Cl)c(Cl)c32)C1] |
| 44 | 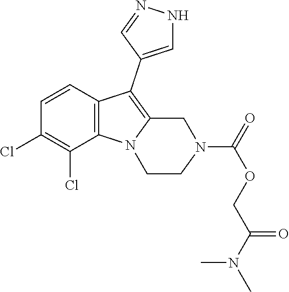 [SMILES: CN(C)C(=O)COC(=O)N1CCn2c(c(-c3cn[nH]c3)c3ccc(Cl)c(Cl)c32)C1] |
| 45 | 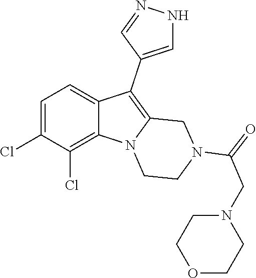 [SMILES: O=C(CN1CCOCC1)N1CCn2c(c(-c3cn[nH]c3)c3ccc(Cl)c(Cl)c32)C1] |
| 46 | 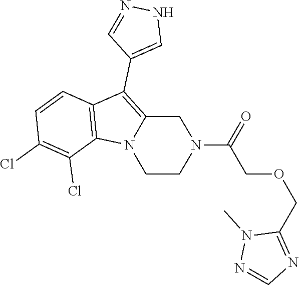 [SMILES: Cn1ncnc1COCC(=O)N1CCn2c(c(-c3cn[nH]c3)c3ccc(Cl)c(Cl)c32)C1] |
| 47 | 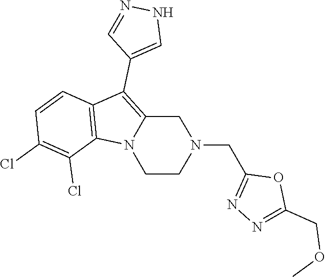 [SMILES: COCc1nnc(CN2CCn3c(c(-c4cn[nH]c4)c4ccc(Cl)c(Cl)c43)C2)o1] |
| 48 | 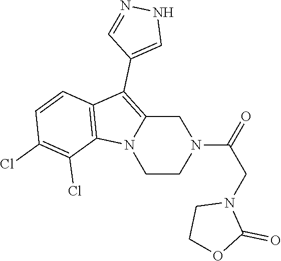 [SMILES: O=C(CN1CCOC1=O)N1CCn2c(c(-c3cn[nH]c3)c3ccc(Cl)c(Cl)c32)C1] |
| 49 | 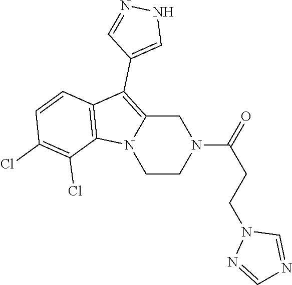 [SMILES: O=C(CCn1cncn1)N1CCn2c(c(-c3cn[nH]c3)c3ccc(Cl)c(Cl)c32)C1] |
| 50 | 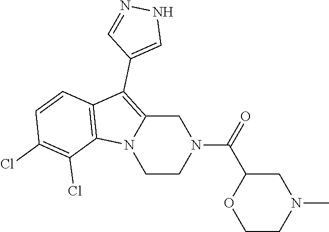 [SMILES: CN1CCOC(C(=O)N2CCn3c(c(-c4cn[nH]c4)c4ccc(Cl)c(Cl)c43)C2)C1] |
| 51 | 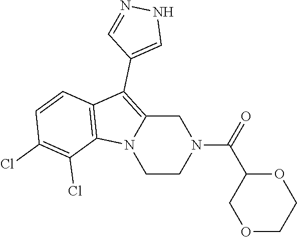 [SMILES: O=C(C1COCCO1)N1CCn2c(c(-c3cn[nH]c3)c3ccc(Cl)c(Cl)c32)C1] |
| 52 | 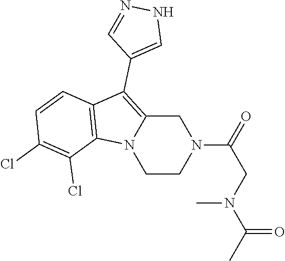 [SMILES: CC(=O)N(C)CC(=O)N1CCn2c(c(-c3cn[nH]c3)c3ccc(Cl)c(Cl)c32)C1] |
| 53 | 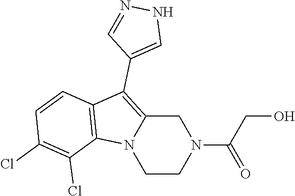 [SMILES: O=C(CO)N1CCn2c(c(-c3cn[nH]c3)c3ccc(Cl)c(Cl)c32)C1] |
| 54 | 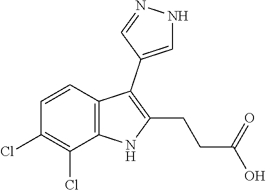 [SMILES: O=C(O)CCc1[nH]c2c(Cl)c(Cl)ccc2c1-c1cn[nH]c1] |
| 55 | 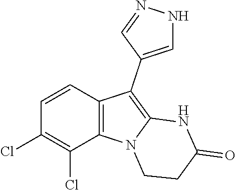 [SMILES: O=C1CCn2c(c(-c3cn[nH]c3)c3ccc(Cl)c(Cl)c32)N1] |
| 56 | 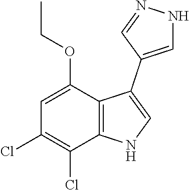 [SMILES: CCOc1cc(Cl)c(Cl)c2[nH]cc(-c3cn[nH]c3)c12] |
| 57 | 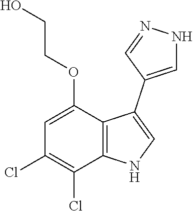 [SMILES: OCCOc1cc(Cl)c(Cl)c2[nH]cc(-c3cn[nH]c3)c12] |
| 58 | 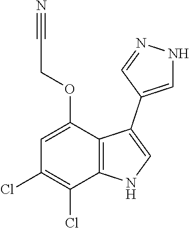 [SMILES: N#CCOc1cc(Cl)c(Cl)c2[nH]cc(-c3cn[nH]c3)c12] |
| 59 | 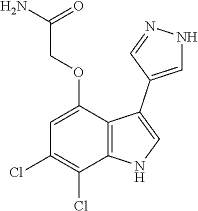 [SMILES: NC(=O)COc1cc(Cl)c(Cl)c2[nH]cc(-c3cn[nH]c3)c12] |
| 60 | 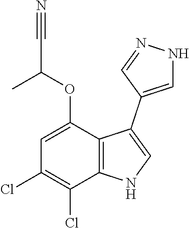 [SMILES: CC(C#N)Oc1cc(Cl)c(Cl)c2[nH]cc(-c3cn[nH]c3)c12] |
| 61 | 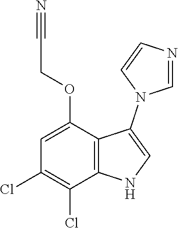 [SMILES: N#CCOc1cc(Cl)c(Cl)c2[nH]cc(-n3ccnc3)c12] |
| 62 | 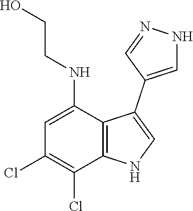 [SMILES: OCCNc1cc(Cl)c(Cl)c2[nH]cc(-c3cn[nH]c3)c12] |
| 63 | 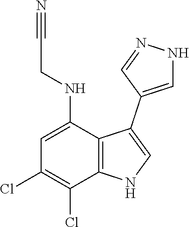 [SMILES: N#CCNc1cc(Cl)c(Cl)c2[nH]cc(-c3cn[nH]c3)c12] |
| 64 | 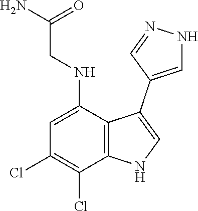 [SMILES: NC(=O)CNc1cc(Cl)c(Cl)c2[nH]cc(-c3cn[nH]c3)c12] |
| 65 | 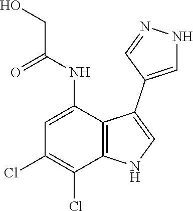 [SMILES: O=C(CO)Nc1cc(Cl)c(Cl)c2[nH]cc(-c3cn[nH]c3)c12] |
| 66 | 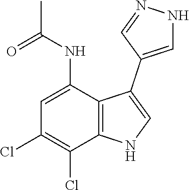 [SMILES: CC(=O)Nc1cc(Cl)c(Cl)c2[nH]cc(-c3cn[nH]c3)c12] |
| 67 | 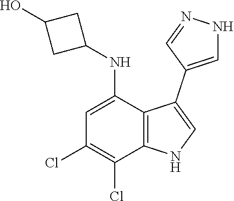 [SMILES: OC1CC(Nc2cc(Cl)c(Cl)c3[nH]cc(-c4cn[nH]c4)c23)C1] |
| 68 | 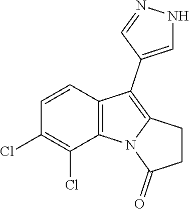 [SMILES: O=C1CCc2c(-c3cn[nH]c3)c3ccc(Cl)c(Cl)c3n21] |
| 69 | 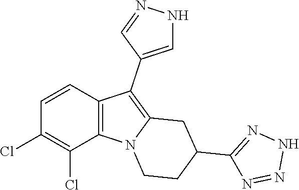 [SMILES: Clc1ccc2c(-c3cn[nH]c3)c3n(c2c1Cl)CCC(c1nn[nH]n1)C3] |
| 70 | 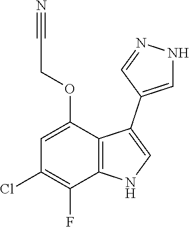 [SMILES: N#CCOc1cc(Cl)c(F)c2[nH]cc(-c3cn[nH]c3)c12] |
| 71 | 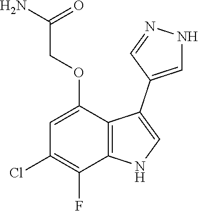 [SMILES: NC(=O)COc1cc(Cl)c(F)c2[nH]cc(-c3cn[nH]c3)c12] |
| 72 | 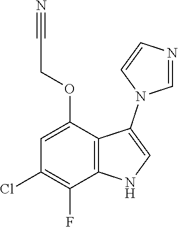 [SMILES: N#CCOc1cc(Cl)c(F)c2[nH]cc(-n3ccnc3)c12] |
| 73 | 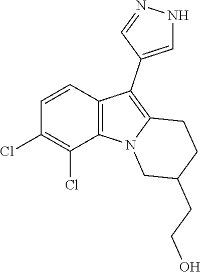 [SMILES: OCCC1CCc2c(-c3cn[nH]c3)c3ccc(Cl)c(Cl)c3n2C1] |
| 74 | 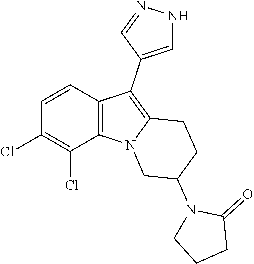 [SMILES: O=C1CCCN1C1CCc2c(-c3cn[nH]c3)c3ccc(Cl)c(Cl)c3n2C1] |
| 75 | 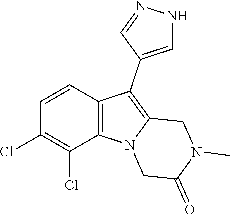 [SMILES: CN1Cc2c(-c3cn[nH]c3)c3ccc(Cl)c(Cl)c3n2CC1=O] |
| 76 | 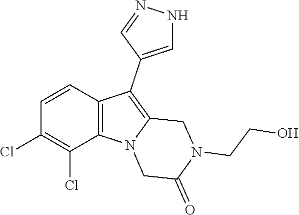 [SMILES: O=C1Cn2c(c(-c3cn[nH]c3)c3ccc(Cl)c(Cl)c32)CN1CCO] |
| 77 | 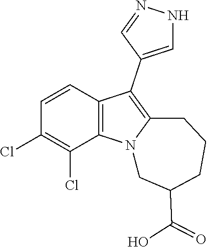 [SMILES: O=C(O)C1CCCc2c(-c3cn[nH]c3)c3ccc(Cl)c(Cl)c3n2C1] |
| 78 | 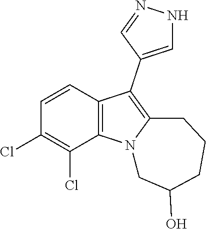 [SMILES: OC1CCCc2c(-c3cn[nH]c3)c3ccc(Cl)c(Cl)c3n2C1] |
| 79 | 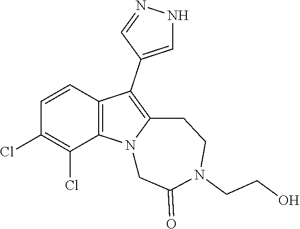 [SMILES: O=C1Cn2c(c(-c3cn[nH]c3)c3ccc(Cl)c(Cl)c32)CCN1CCO] |
| 80 | 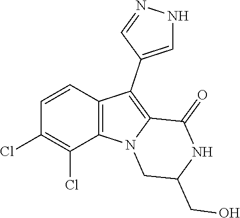 [SMILES: O=C1NC(CO)Cn2c1c(-c1cn[nH]c1)c1ccc(Cl)c(Cl)c12] |
| 81 | 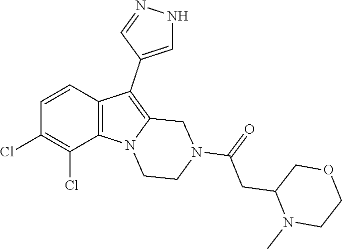 [SMILES: CN1CCOCC1CC(=O)N1CCn2c(c(-c3cn[nH]c3)c3ccc(Cl)c(Cl)c32)C1] |
| 82 | 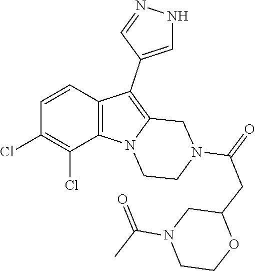 [SMILES: CC(=O)N1CCOC(CC(=O)N2CCn3c(c(-c4cn[nH]c4)c4ccc(Cl)c(Cl)c43)C2)C1] |
| 83 | 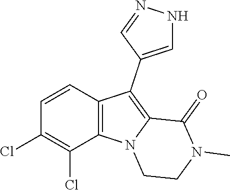 [SMILES: CN1CCn2c(c(-c3cn[nH]c3)c3ccc(Cl)c(Cl)c32)C1=O] |
| 84 | 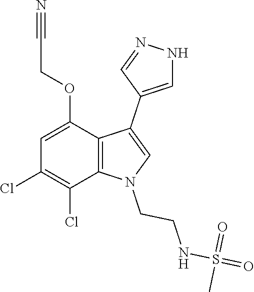 [SMILES: CS(=O)(=O)NCCn1cc(-c2cn[nH]c2)c2c(OCC#N)cc(Cl)c(Cl)c21] |
| 85 | 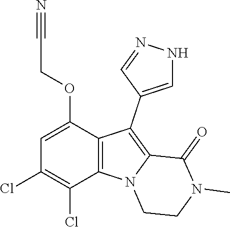 [SMILES: CN1CCn2c(c(-c3cn[nH]c3)c3c(OCC#N)cc(Cl)c(Cl)c32)C1=O] |
| 86 | 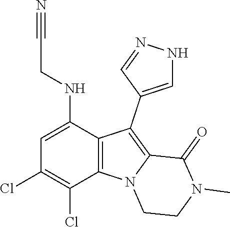 [SMILES: CN1CCn2c(c(-c3cn[nH]c3)c3c(NCC#N)cc(Cl)c(Cl)c32)C1=O] |
| 87 | 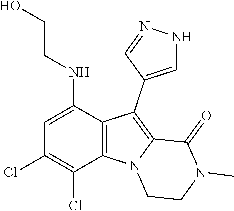 [SMILES: CN1CCn2c(c(-c3cn[nH]c3)c3c(NCCO)cc(Cl)c(Cl)c32)C1=O] |
| 88 | 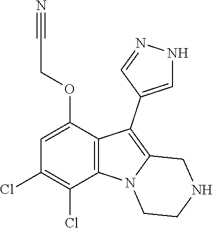 [SMILES: N#CCOc1cc(Cl)c(Cl)c2c1c(-c1cn[nH]c1)c1n2CCNC1] |
| 89 | 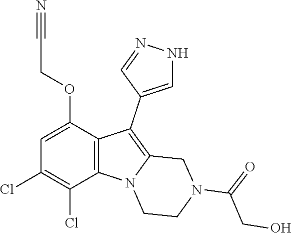 [SMILES: N#CCOc1cc(Cl)c(Cl)c2c1c(-c1cn[nH]c1)c1n2CCN(C(=O)CO)C1] |
| 90 | 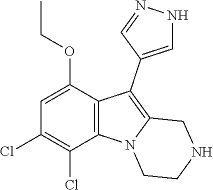 [SMILES: CCOc1cc(Cl)c(Cl)c2c1c(-c1cn[nH]c1)c1n2CCNC1] |
| 91 | 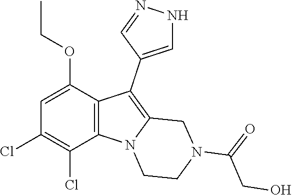 [SMILES: CCOc1cc(Cl)c(Cl)c2c1c(-c1cn[nH]c1)c1n2CCN(C(=O)CO)C1] |
| 92a | 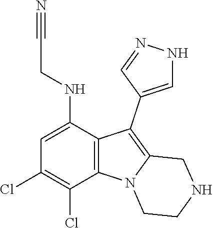 [SMILES: N#CCNc1cc(Cl)c(Cl)c2c1c(-c1cn[nH]c1)c1n2CCNC1] |
| 92 | 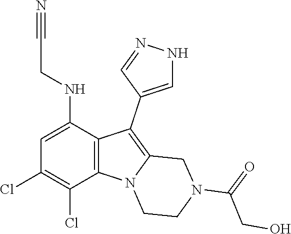 [SMILES: N#CCNc1cc(Cl)c(Cl)c2c1c(-c1cn[nH]c1)c1n2CCN(C(=O)CO)C1] |
| 93 | 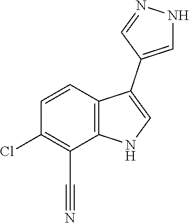 [SMILES: N#Cc1c(Cl)ccc2c(-c3cn[nH]c3)c[nH]c12] |
| 94 | 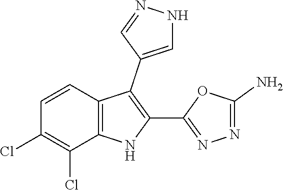 [SMILES: Nc1nnc(-c2[nH]c3c(Cl)c(Cl)ccc3c2-c2cn[nH]c2)o1] |

### Intermediate 244

### N-[3,4-Dichloro-10-(1-tetrahydropyran-2-ylpyrazol-4-yl)-6, 7,8, 9-tetrahydropyrido[1,2-a]indol-7-yl]-2-hydroxy-acetamide

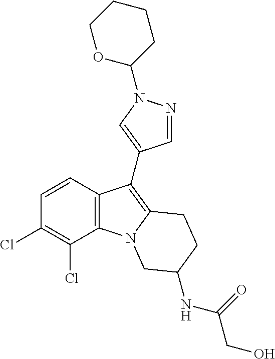 [SMILES: O=C(CO)NC1CCc2c(-c3cnn(C4CCCCO4)c3)c3ccc(Cl)c(Cl)c3n2C1]

Dissolve 3,4-dichloro-10-(1-tetrahydropyran-2-ylpyrazol-4-yl)-6,7,8,9-tetrahydropyrido[1,2-a]indol-7-amine (100 mg, 0.234 mmol), glycolic acid (38 mg, 0.47 mmol), DIPEA (0.21 mL, 1.2 mmol) and HATU (182 mg, 0.469 mmol) in DMF (3 mL), then stir at ambient temperature for 16-18 hrs. Concentrate under vacuum, dissolve the residue in EtOAc, wash with water, and extract the aqueous layer with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give N-[3,4-dichloro-10-(1-tetrahydropyran-2-ylpyrazol-4-yl)-6,7,8,9-tetrahydropyrido[1,2-a]indol-7-yl]-2-hydroxy-acetamide (96 mg, 66%) as a white solid. ES/MS (m/z): (³⁵Cl/³⁷Cl) 463.0/465.0 (M+H) and 378.9/381.0 (M+H-THP).

### EXAMPLE 95

### N-[3,4-Dichloro-10-(1H-pyrazol-4-yl)-6,7,8,9-tetrahydropyrido[1,2-a]indol-7-yl]-2-hydroxy-acetamide

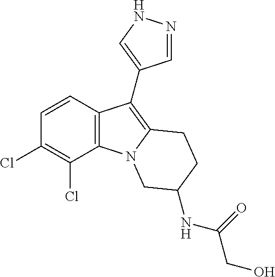 [SMILES: O=C(CO)NC1CCc2c(-c3cn[nH]c3)c3ccc(Cl)c(Cl)c3n2C1]

Dissolve N-[3,4-dichloro-10-(1-tetrahydropyran-2-ylpyrazol-4-yl)-6,7,8,9-tetrahydropyrido[1,2-a]indol-7-yl]-2-hydroxy-acetamide (96 mg, 0.16 mmol) in 4M HCl/dioxane (3 mL) and stir at ambient temperature for 1 hr. Concentrate under vacuum and purify by prep-HPLC to give N-[3,4-dichloro-10-(1H-pyrazol-4-yl)-6,7,8,9-tetrahydropyrido[1,2-a]indol-7-yl]-2-hydroxy-acetamide (22.99 mg, 39%) as a white solid. ES/MS (m/z): (³⁵Cl/³⁷Cl) 379.3/381.3 (M+H).

### Intermediate 245

### N-[3,4-Dichloro-10-(1-tetrahydropyran-2-ylpyrazol-4-yl)-6, 7,8, 9-tetrahydropyrido[1,2-a]indol-7-yl]tetrahydropyran-4-carboxamide

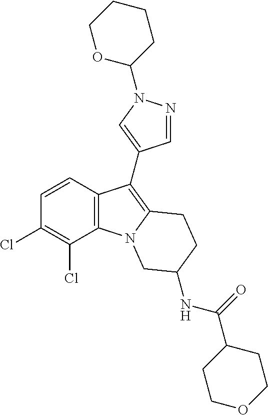 [SMILES: O=C(NC1CCc2c(-c3cnn(C4CCCCO4)c3)c3ccc(Cl)c(Cl)c3n2C1)C1CCOCC1]

Dissolve 3,4-dichloro-10-(1-tetrahydropyran-2-ylpyrazol-4-yl)-6,7, 8,9-tetrahydropyrido[1,2-a]indol-7-amine (100 mg, 0.234 mmol), tetrahydro-2H-pyran-4-carboxylic acid (62 mg, 0.48 mmol), DIPEA (0.21 mL, 1.2 mmol) and HATU (182 mg, 0.469 mmol) in DMF (2 mL). Stir at ambient temperature for 16-18 hrs. Dilute with EtOAc, wash with water, and extract the aqueous layer with EtOAc (3×). Wash the combined organic layers with saturated aqueous NaCl, dry over anhydrous sodium sulfate, filter, and concentrate under vacuum. Purify by flash column chromatography eluting with MeOH in DCM to give N-[3,4-dichloro-10-(1-tetrahydropyran-2-ylpyrazol-4-yl)-6,7,8,9-tetrahydropyrido[1,2-a]indol-7-yl]tetrahydropyran-4-carboxamide (216 mg, 99+%) as a white solid. ES/MS (m/z): (³⁵Cl/³⁷Cl) 517.2/519.2 (M+H) and 433.1/435.1 (M+H-THP).

### EXAMPLE 96

### N-[3,4-Dichloro-10-(1H-pyrazol-4-yl)-6,7,8,9-tetrahydropyrido[1,2-a]indol-7-yl]tetrahydropyran-4-carboxamide

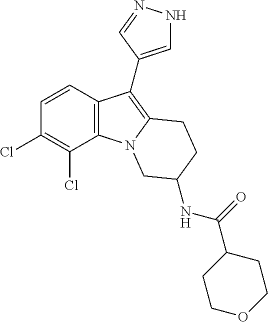 [SMILES: O=C(NC1CCc2c(-c3cn[nH]c3)c3ccc(Cl)c(Cl)c3n2C1)C1CCOCC1]

Suspend N-[3,4-dichloro-10-(1-tetrahydropyran-2-ylpyrazol-4-yl)-6,7,8,9-tetrahydropyrido[1,2-a]indol-7-yl]tetrahydropyran-4-carboxamide (216 mg, 0.334 mmol) in 4M HCl/dioxane (5 mL) and stir at ambient temperature for 2 hrs. Concentrate under vacuum and purify by prep-HPLC to give N-[3,4-dichloro-10-(1H-pyrazol-4-yl)-6,7,8,9-tetrahydropyrido[1,2-a]indol-7-yl]tetrahydropyran-4-carboxamide (37.43 mg, 26%) as a white solid. ES/MS (m/z): (³⁵Cl/³⁷Cl) 433.3/435.3 (M+H).

### Intermediate 252

### 4-Bromo-6,7-dichloro-3-(1-(tetrahydro-2H-pyran-2-yl)-1H-pyrazol-4-yl)-1H-indole

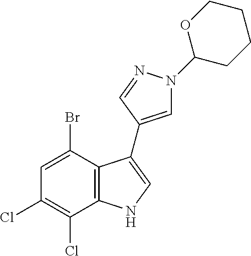 [SMILES: Clc1cc(Br)c2c(-c3cnn(C4CCCCO4)c3)c[nH]c2c1Cl]

Dissolve 4-bromo-6,7-dichloro-1-(p-tolylsulfonyl)-3-(1-tetrahydropyran-2-ylpyrazol-4-yl)indole (5.40 g, 8.80 mmol) in THF (15 mL). Add TBAF (26 mL, 26.0 mmol, 1.0M in THF) and stir at ambient temperature for 3 hrs. Dilute with EtOAc, wash with saturated aqueous NH₄Cl, dry over anhydrous Na₂SO₄, filter, and concentrate under vacuum. Purify by column chromatography eluting with EtOAc in petroleum ether to give 4-bromo-6,7-dichloro-3-(1-(tetrahydro-2H-pyran-2-yl)-1H-pyrazol-4-yl)-1H-indole (3.15 g, 76%) as a yellow solid. ES/MS (m/z): 414.0/416.0/417.9 (M+H) and 330.0/332.0/334.0 (M+H-THP).

### Intermediate 253

### 4-Bromo-6,7-dichloro-3-(1-(tetrahydro-2H-pyran-2-yl)-1H-pyrazol-4-yl)-1-((2-(trimethylsilyl)ethoxy)methyl)-1H-indole

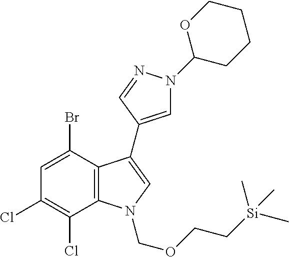 [SMILES: C[Si](C)(C)CCOCn1cc(-c2cnn(C3CCCCO3)c2)c2c(Br)cc(Cl)c(Cl)c21]

Dissolve 4-bromo-6,7-dichloro-3-(1-(tetrahydro-2H-pyran-2-yl)-1H-pyrazol-4-yl)-1H-indole (3.15 g, 6.68 mmol) in THF (5 mL). Add NaH (801 mg, 20.0 mmol, 60% dispersion in mineral oil) at 0° C. and stir for 30 min. Add SEM-Cl (1.5 mL, 8.10 mmol) at 0° C. and stir at ambient temperature for 16-18 hrs. Combine with another batch run at 0.90×scale for work up and purification. Dilute with EtOAc, wash with saturated aqueous NaCl, dry over anhydrous Na₂SO₄, filter, and concentrate under vacuum. Purify by column chromatography eluting with EtOAc in petroleum ether to give 4-bromo-6,7-dichloro-3-(1-(tetrahydro-2H-pyran-2-yl)-1H-pyrazol-4-yl)-1-((2-(trimethylsilyl)ethoxy)methyl)-1H-indole (5.97 g, 80%) as yellow oil. ES/MS (m/z): 544.1/546.0/548.0 (M+H).

### Intermediate 254

### 6,7-Dichloro-3-(1-tetrahydropyran-2-ylpyrazol -4-yl)-1-(2-trimethylsilylethoxymethyl)indol-4-ol

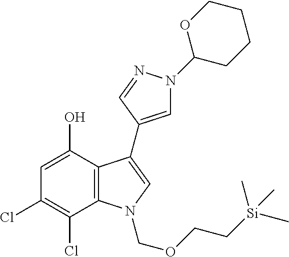 [SMILES: C[Si](C)(C)CCOCn1cc(-c2cnn(C3CCCCO3)c2)c2c(O)cc(Cl)c(Cl)c21]

Dissolve 4-bromo-6,7-dichloro-3-(1-(tetrahydro-2H-pyran-2-yl)-1H-pyrazol-4-yl)-1-((2-(trimethylsilyl)ethoxy)methyl)-1H-indole (1.00 g, 1.71 mmol) in dioxane (8 mL) and water (2 mL). Add t-BuBrettPhos-Pd-G₃ (152 mg, 0.171 mmol) and t-BuONa (410 mg, 4.27 mmol) under N₂. Purge with N₂ and stir at 70° C. for 2 hrs. Quench with saturated aqueous NH₄Cl and extract with EtOAc. Wash with saturated aqueous NaCl, dry over anhydrous Na₂SO₄, filter, and concentrate under vacuum. Purify by column chromatography eluting with EtOAc in petroleum ether to give 6,7-dichloro-3-(1-tetrahydropyran-2-ylpyrazol-4-yl)-1-(2-trimethylsilylethoxymethyl)indol-4-ol (264 mg, 18%) as a brown solid. ES/MS (m/z): (³⁵Cl/³⁷Cl) 482.2/484.1 (M+H).

### Intermediate 255

### 4-((trans-3-((tert-Butyldimethylsilyl)oxy)cyclopentyl)oxy)-6,7-dichloro-3-(1-(tetrahydro-2H-pyran-2-yl)-1H-pyrazol-4-yl)-1-((2-(trimethyl silyl)ethoxy)methyl)-1H-indole

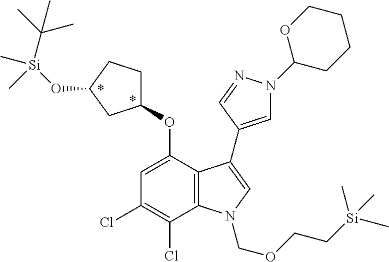

Dissolve 6,7-dichloro-3-(1-tetrahydropyran-2-ylpyrazol-4-yl)-1-(2-trimethylsilylethoxymethyl)indol-4-ol (160 mg, 0.312 mmol), cis-3-((tert-butyldimethylsilyl)oxy)cyclopentan-1-ol (CAS: 183612-97-7, 150 mg, 0.679 mmol) and Ph₃P (210 mg, 0.785 mmol) in toluene (5 mL). Add DIAD (0.1 mL, 0.47 mmol) and stir at 110° C. for 16-18 hrs under N₂ atmosphere. Quench with water and extract with EtOAc. Wash with saturated aqueous NaCl, dry over anhydrous Na₂SO₄, filter, and concentrate under vacuum. Purify using column chromatography eluting with EtOAc in petroleum ether to give 4-((trans-3-((tert-butyldimethylsilyl)oxy)cyclopentyl)oxy)-6,7-dichloro-3-(1-(tetrahydro-2H-pyran-2-yl)-1H-pyrazol-4-yl)-1-((2-(trimethylsilyl)ethoxy)methyl)-1H-indole (210 mg, 81%) as a yellow solid. The material is a racemic mixture. ES/MS (m/z): 681.8 (M+H).

### EXAMPLE 99

### trans-3-((6,7-Dichloro-3-(1H-pyrazol-4-yl)-1H-indol-4-yl)oxy)cyclopentan-1-ol (racemic mixture)

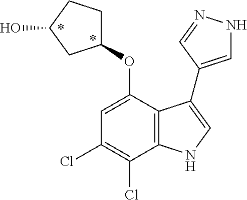 [SMILES: O[C@@H]1CC[C@@H](Oc2cc(Cl)c(Cl)c3[nH]cc(-c4cn[nH]c4)c23)C1]

Dissolve 4-((trans-3-((tert-butyldimethyl silyl)oxy)cyclopentyl)oxy)-6, 7-dichloro-3-(1-(tetrahydro-2H-pyran-2-yl)-1H-pyrazol-4-yl)-1-((2-(trimethylsilyl)ethoxy)methyl)-1H-indole (210 mg, 0.253 mmol) and ethylenediamine (0.17 mL, 2.53 mmol) in TBAF (3 mL, 3.00 mmol, 1.0M in THF). Stir at 80° C. for 16-18 hrs. Dilute with EtOAc, wash with saturated aqueous NH₄Cl, saturated aqueous NaCl, dry over anhydrous Na₂SO₄, filter, and concentrate under vacuum. Dissolve the residue in THF (2 mL). Add 6M aqueous HCl (2 mL). Then stir at ambient temperature for 2 hrs. Dilute with water, extract with EtOAc and combine the organic layers. Wash with saturated aqueous NaCl, dry over anhydrous Na₂SO₄, filter, and concentrate under vacuum. Purify by reverse phase prep-HPLC to give trans-3-((6,7-dichloro-3-(1H-pyrazol-4-yl)-1H-indol-4-yl)oxy)cyclopentan-1-ol (15.32 mg, 22%) as a white solid. ES/MS (m/z): (³⁵Cl/³⁷Cl) 352.2/354.2 (M+H).

### Intermediate 256

### 4-((cis-3-((tert-Butyldimethylsilyl)oxy)cyclopentyl)oxy)-6,7-dichloro-3-(1-(tetrahydro-2H-pyran-2-yl)-1H-pyrazol-4-yl)-14(2-(trimethylsilyl)ethoxy)methyl)-1H-indole

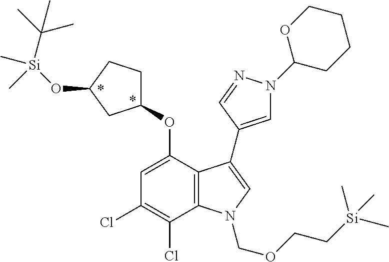

Dissolve 6,7-dichloro-3-(1-tetrahydropyran-2-ylpyrazol-4-yl)-1-(2-trimethylsilylethoxymethyl)indol-4-ol (200 mg, 0.236 mmol, 57% purity), trans-3-((tert-butyldimethylsilyl)oxy)cyclopentan-1-ol (CAS: 2093272-13-8, 110 mg, 0.498 mmol) and Ph₃P (160 mg, 0.598 mmol) in toluene (5 mL). Add DIAD (0.080 mL, 0.38 mmol) and stir at 110° C. for 16-18 hrs under N₂. Combine with another batch run at 0.25×scale for work up and purification. Quench with water and extract with EtOAc. Wash with saturated aqueous NaCl, dry over anhydrous Na₂SO₄, filter, and concentrate under vacuum. Purify using column chromatography eluting with EtOAc in petroleum ether to give 4-((cis-3-((tert-butyldimethylsilyl)oxy)cyclopentyl)oxy)-6,7-dichloro-3-(1-(tetrahydro-2H-pyran-2-yl)-1H-pyrazol-4-yl)-1-((2-(trimethylsilyl)ethoxy)methyl)-1H-indole (200 mg, 92%) as a yellow solid. The material is a racemic mixture.

### EXAMPLE 100

### Cis-3-((6,7-Dichloro-3-(1H-pyrazol-4-yl)-1H-indol-4-yl)oxy)cyclopentan-1-ol (racemic mixture)

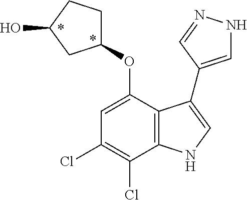 [SMILES: O[C@H]1CC[C@@H](Oc2cc(Cl)c(Cl)c3[nH]cc(-c4cn[nH]c4)c23)C1]

Dissolve 4-((cis-3-((tert-butyldimethylsilyl)oxy)cyclopentyl)oxy)-6,7-dichloro-3-(1-(tetrahydro-2H-pyran-2-yl)-1H-pyrazol-4-yl)-1-((2-(trimethylsilyl)ethoxy)methyl)-1H-indole (170 mg, 0.232 mmol) and ethylenediamine (0.19 mL, 2.83 mmol) in TBAF (3 mL, 3.00 mmol, 1.0M in THF). Stir at 80° C. for 16-18 hrs. Dilute with EtOAc, wash with saturated aqueous NH₄Cl, saturated aqueous NaCl, dry over anhydrous Na₂SO₄, filter, and concentrate under vacuum. Dissolve the residue in DCM (2 mL). Add TFA (2 mL). Then stir at ambient temperature for 2 hrs. Concentrate and dissolve in EtOH (3 mL). Add K₂CO₃ (100 mg, 0.716 mmol) and stir at 85° C. for 1.5 hrs. Dilute with water, extract with EtOAc. Wash with saturated aqueous NaCl, dry over anhydrous Na₂SO₄, filter, and concentrate under vacuum. Purify by prep-HPLC to give cis-3-((6,7-dichloro-3-(1H-pyrazol-4-yl)-1H-indol-4-yl)oxy)cyclopentan-1-ol (19.96 mg, 25%) as a white solid. The compound is a racemic mixture. ES/MS (m/z): (³⁵Cl/³⁷Cl) 352.2/354.2 (M+H).

### Intermediate 257

### 6,7-Dichloro-1-(p-tolylsulfonyl)indole

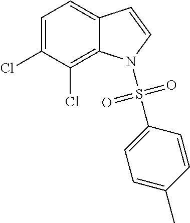 [SMILES: Cc1ccc(S(=O)(=O)n2ccc3ccc(Cl)c(Cl)c32)cc1]

Suspend 6,7-dichloro-1H-indole (3 g, 15 mmol) in THF (30 mL). Add sodium hydride (1.54 g, 38.5 mmol, 60% dispersion in oil) at 0° C. After stirring for an hr, add 4-methylbenzenesulfonyl chloride (5.85 g, 30.7 mmol) at 0° C., then stir at 25° C. for 16-18 hrs. Dilute with saturated aqueous NH₄Cl and extract with EtOAc. Wash with saturated aqueous NaCl, dry over anhydrous sodium sulfate, filter, and concentrate under vacuum. Purify by column chromatography eluting with EtOAc in petroleum ether to give 6,7-dichloro-1-(p-tolylsulfonyl)indole (4.97 g, 86%). ES/MS (m/z): (³⁵Cl/³⁷Cl) 339.8/341.8 (M+H).

### Intermediate 258

### tert-Butyl (1-((tert-butyl dimethyl silyl)oxy)-3-(6,7-dichloro-1-tosyl-1H-indol-2-yl)propan-2-yl)carbamate

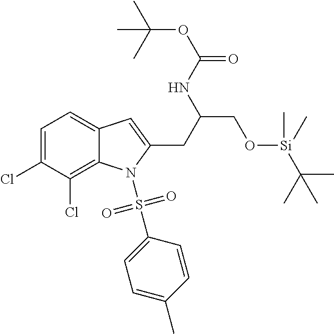 [SMILES: Cc1ccc(S(=O)(=O)n2c(CC(CO[Si](C)(C)C(C)(C)C)NC(=O)OC(C)(C)C)cc3ccc(Cl)c(Cl)c32)cc1]

Dissolve 6,7-dichloro-1-(p-tolylsulfonyl)indole (2.00 g, 5.60 mmol) in THF (20 mL) and cool to −78° C. Add n-BuLi (3.40 mL, 8.50 mmol, 2.5M in hexane) dropwise and stir 1 hr. Add a solution of tert-butyl 4-(((tert-butyldimethylsilyl)oxy)methyl)-1,2,3-oxathiazolidine-3-carboxylate 2,2-dioxide (CAS: 2386255-17-8, 3.10 g, 8.27 mmol) in THF (10 mL) dropwise at −78° C. and then warm to ambient temperature slowly and stir for 16-18 hrs. Quench with saturated aqueous NH₄Cl and extract with EtOAc. Wash with saturated aqueous NaCl, dry over anhydrous sodium sulfate, filter, and concentrate under vacuum. Purify by column chromatography eluting with EtOAc in petroleum ether to give tert-butyl (1-((tert-butyldimethyl silyl)oxy)-3-(6,7-dichloro-1-tosyl-1H-indol-2-yl)propan-2-yl)carbamate (1.50 g, 39%) as a yellow gum. ES/MS (m/z): (³⁵Cl/³⁷Cl) 627.1/629.2 (M+H) and 527.1/529.1 (M+H-Boc).

### Intermediate 259

### tert-Butyl (1-(6,7-dichloro-1H-indol-2-yl)-3-hydroxypropan-2-yl)carbamate

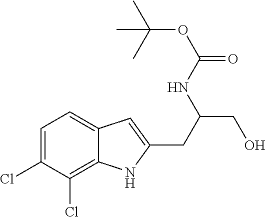 [SMILES: CC(C)(C)OC(=O)NC(CO)Cc1cc2ccc(Cl)c(Cl)c2[nH]1]

Dissolve tert-butyl (1-((tert-butyldimethylsilyl)oxy)-3-(6,7-dichloro-1-tosyl-1H-indol-2-yl)propan-2-yl)carbamate (1.50 g, 2.15 mmol) in THF (20 mL). Add TBAF (10 mL, 10.0 mmol, 1.0M in THF) and stir at 80° C. for 16-18 hrs. Cool to ambient temperature, dilute with EtOAc, wash with saturated aqueous NH₄Cl, saturated aqueous NaCl, dry over anhydrous sodium sulfate, filter, and concentrate under vacuum. Purify by column chromatography eluting with EtOAc in petroleum ether to give tert-butyl (1-(6,7-dichloro-1H-indol-2-yl)-3-hydroxypropan-2-yl)carbamate (300 mg, 37%) as a yellow solid. ES/MS (m/z): (³⁵Cl/³⁷Cl) 359.0/361.0 (M+H) and 302.9/304.9 (M+H-tBu).

### Intermediate 260

### tert-Butyl (1-(6,7-dichloro-3-iodo-1H-indol-2-yl)-3-hydroxypropan-2-yl)carbamate

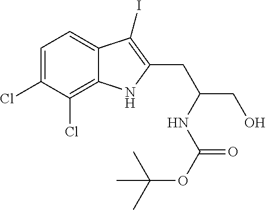 [SMILES: CC(C)(C)OC(=O)NC(CO)Cc1[nH]c2c(Cl)c(Cl)ccc2c1I]

Dissolve tert-butyl (1-(6,7-dichloro-1H-indol-2-yl)-3-hydroxypropan-2-yl)carbamate (250 mg, 0.654 mmol) in DMF (4 mL). Add NIS (160 mg, 0.697 mmol) at 0° C. and stir at ambient temperature for 1 hr. Dilute with EtOAc, wash with water, saturated aqueous NaCl, dry over anhydrous sodium sulfate, filter, and concentrate under vacuum. Purify by column chromatography eluting with EtOAc in petroleum ether to give tert-butyl (1-(6,7-dichloro-3-iodo-1H-indol-2-yl)-3-hydroxypropan-2-yl)carbamate (250 mg, 72%) as a yellow solid. ES/MS (m/z): 484.8 (M+H) and 428.8/430.9 (M+H-tBu).

### Intermediate 261

### tert-Butyl (1-(6, 7-dichloro-3-(1-(tetrahydro-2H-pyran-2-yl)-1H-pyrazol-4-yl)-1H-indol -2-yl)-3-hydroxypropan-2-yl)carbamate

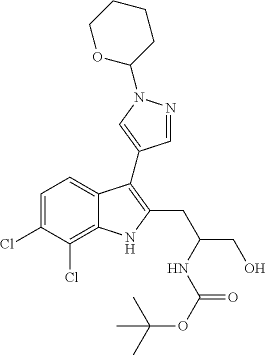 [SMILES: CC(C)(C)OC(=O)NC(CO)Cc1[nH]c2c(Cl)c(Cl)ccc2c1-c1cnn(C2CCCCO2)c1]

Dissolve tert-butyl (1-(6,7-dichloro-3-iodo-1H-indol-2-yl)-3-hydroxypropan-2-yl)carbamate (250 mg, 0.464 mmol), 1-tetrahydropyran-2-yl-4-(4,4,5,5-tetramethyl-1,3,2-dioxaborolan-2-yl) pyrazole (200 mg, 0.719 mmol), Pd(dtbpf)Cl₂ (60 mg, 0.090 mmol) in 1,4-dioxane (5 mL) and water (1.25 mL). Add sodium carbonate (173 mg, 1.62 mmol) and purge with N₂. Stir at 90° C. for 4 hrs. Combine with another batch run at 0.15×scale for work up and purification. Concentrate under vacuum and purify by column chromatography eluting with MeOH in DCM to give tert-butyl (1-(6,7-dichloro-3-(1-(tetrahydro-2H-pyran-2-yl)-1H-pyrazol-4-yl)-1H-indol-2-yl)-3-hydroxypropan-2-yl)carbamate (250 mg, 83%) as a light-yellow solid.

### Intermediate 262

### Ethyl 2-(2-(2-((tert-butoxycarbonyl)amino)-3-hydroxypropyl)-6, 7-dichloro-3-(1-(tetrahydro-2H-pyran-2-yl)-1H-pyrazol-4-yl)-1H-indol-1-yl)acetate

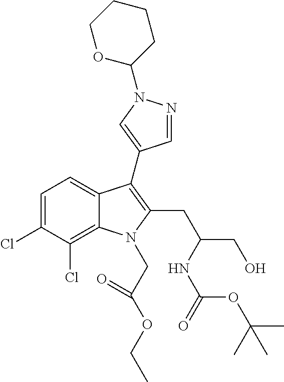 [SMILES: CCOC(=O)Cn1c(CC(CO)NC(=O)OC(C)(C)C)c(-c2cnn(C3CCCCO3)c2)c2ccc(Cl)c(Cl)c21]

Dissolve tert-butyl (1-(6, 7-dichloro-3-(1-(tetrahydro-2H-pyran-2-yl)-1H-pyrazol-4-yl)-1H-indol-2-yl)-3-hydroxypropan-2-yl)carbamate (170 mg, 0.300 mmol) in DMF (3 mL). Add Cs₂CO₃ (200 mg, 0.614 mmol), then ethyl bromoacetate (0.050 mL, 0.50 mmol) at 0° C. and stir at ambient temperature for 5 hrs. Combine with another batch run at 0.47×scale for work up and purification. Dilute with EtOAc, wash with water, saturated aqueous NaCl, dry over anhydrous sodium sulfate, filter, and concentrate under vacuum. Purify using column chromatography eluting with MeOH in DCM to give ethyl 2-(2-(2-((tert-butoxycarbonyl)amino)-3-hydroxypropyl)-6, 7-dichloro-3-(1-(tetrahydro-2H-pyran-2-yl)-1H-pyrazol-4-yl)-1H-indol-1-yl)acetate (90 mg, 28%) as a white solid. ES/MS (m/z): (³⁵Cl/³⁷Cl) 595.2/597.2 (M+H).

### EXAMPLE 101

### 7,8-Dichloro-2-(hydroxy methyl)-11-(1H-pyrazol-4-yl)-2,3-dihydro-1H-[1,4]diazepino[1,7-a]indol-4(5H)-one

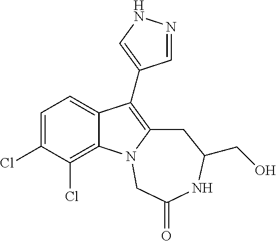 [SMILES: O=C1Cn2c(c(-c3cn[nH]c3)c3ccc(Cl)c(Cl)c32)CC(CO)N1]

Dissolve ethyl 2-(2-(2-((tert-butoxycarbonyl)amino)-3-hydroxypropyl)-6,7-dichloro-3-(1-(tetrahydro-2H-pyran-2-yl)-1H-pyrazol-4-yl)-1H-indol-1-yl)acetate (70 mg, 0.096 mmol) in DCM (3 mL). Add TFA (1.5 mL) and stir at ambient temperature for 1 hr. Combine with another batch run at 0.29×scale for work up and purification. Concentrate under vacuum and dilute the residue with water, basify with saturated aqueous sodium bicarbonate to pH=8, extract with EtOAc, wash with saturated aqueous NaCl, dry over anhydrous sodium sulfate, filter, and concentrate under vacuum. Dissolve the residue into ethanol (4 mL), add potassium carbonate (80 mg, 0.58 mmol) and stir at 90° C. for 1.5 hrs. Cool to ambient temperature, dilute with water, extract with EtOAc, dry over anhydrous sodium sulfate, filter, and concentrate under vacuum. Purify by reverse phase prep-HPLC to give 7,8-dichloro-2-(hydroxymethyl)-11-(1H-pyrazol-4-yl)-2,3-dihydro-1H-[1,4]diazepino[1,7-a]indol-4(5H)-one (25.43 mg, 55%) as a white solid. ES/MS (m/z): (³⁵Cl/³⁷Cl) 365.3/367.3 (M+H).

### Intermediate 263

### Methyl 3-(4-bromo-6,7-dichloro-1H-indol-2-yl)propanoate

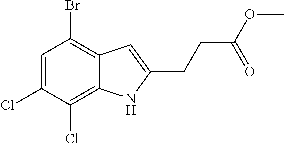 [SMILES: COC(=O)CCc1cc2c(Br)cc(Cl)c(Cl)c2[nH]1]

Suspend 4-bromo-6,7-dichloro-1H-indole (5.00 g, 18.9 mmol), methyl 3-bromopropanoate (3.86 g, 22.7 mmol), Pd(PhCN)₂Cl₂ (1.15 g, 2.85 mmol), norborn-2-ene (7.18 g, 75.5 mmol) and sodium bicarbonate (6.37 g, 75.4 mmol) in water (510 mg, 28.3 mmol) and DMF (50 mL). Purge with N₂ and heat to 70° C. for 16-18 hrs. Cool to ambient temperature, dilute with EtOAc, wash with water and saturated aqueous NaCl, dry over anhydrous sodium sulfate, filter, and concentrate under vacuum. Purify by column chromatography eluting with EtOAc in petroleum ether to give methyl 3-(4-bromo-6,7-dichloro-1H-indol-2-yl)propanoate (3.70 g, 53%) as a yellow solid.

### Intermediate 264

### Methyl 3-(4-bromo-1-(2-(tert-butoxy)-2-oxo ethyl)-6,7-dichloro-1H-indol-2-yl)propanoate

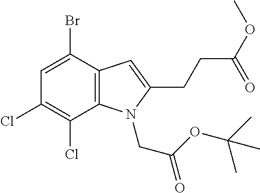 [SMILES: COC(=O)CCc1cc2c(Br)cc(Cl)c(Cl)c2n1CC(=O)OC(C)(C)C]

Dissolve methyl 3-(4-bromo-6,7-dichloro-1H-indol-2-yl)propanoate (3.70 g, 10.0 mmol) in DMF (50 mL). Add Cs₂CO₃ (11.0 g, 33.7 mmol) and tert-butyl 2-bromoacetate (3.90 g, 20.0 mmol) at 0° C. Stir at ambient temperature for 3 hrs. Dilute with EtOAc, wash with water and saturated aqueous NaCl, dry over anhydrous sodium sulfate, filter, and concentrate under vacuum. Purify by column chromatography eluting with EtOAc in petroleum ether to give methyl 3-(4-bromo-1-(2-(tert-butoxy)-2-oxoethyl)-6,7-dichloro-1H-indol-2-yl)propanoate (4.00 g, 82%) as a yellow solid.

### Intermediate 265

### tert-Butyl 1-bromo-3,4-dichloro-7-oxo-8, 9-dihydro-6H-pyrido[1,2-a]indole-6-carboxylate

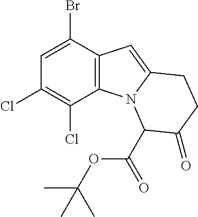 [SMILES: CC(C)(C)OC(=O)C1C(=O)CCc2cc3c(Br)cc(Cl)c(Cl)c3n21]

Dissolve methyl 3-(4-bromo-1-(2-(tert-butoxy)-2-oxoethyl)-6,7-dichloro-1H-indol-2-yl)propanoate (3.20 g, 6.50 mmol) in THF (30 mL) and add potassium tert-butoxide in THF (26 mL, 26.0 mmol, 1M in THF). Stir at 28° C. for 3 hrs., under N₂ atmosphere. Quench with saturated aqueous NH₄Cl, extract with EtOAc, dry over anhydrous sodium sulfate, filter, and concentrate under vacuum. Purify by column chromatography eluting with EtOAc in petroleum ether to give tert-butyl 1-bromo-3,4-dichloro-7-oxo-8,9-dihydro-6H-pyrido[1,2-a]indole-6-carboxylate (2.20 g, 74%) as a yellow solid.

### Intermediate 266

### 1-Bromo-3,4-dichloro-8,9-dihydro-6H-pyrido[1,2-a]indol-7-one

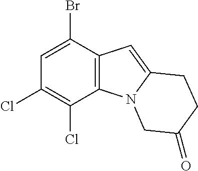 [SMILES: O=C1CCc2cc3c(Br)cc(Cl)c(Cl)c3n2C1]

Suspend tert-butyl 1-bromo-3,4-dichloro-7-oxo-8,9-dihydro-6H-pyrido[1,2-a]indole-6-carboxylate (2.20 g, 4.80 mmol) and silica gel (2.00 g, 33.3 mmol) in toluene (20 mL). Purge with N₂ and heat to reflux for 3 hrs. Cool to ambient temperature and concentrate under vacuum. Purify by column chromatography eluting with EtOAc in petroleum ether to give 1-bromo-3,4-dichloro-8,9-dihydro-6H-pyrido[1,2-a]indol-7-one (1.35 g, 84%) as a yellow solid.

### Intermediate 267

### 1-Bromo-3,4-dichloro-6,7,8,9-tetrahydropyrido[1,2-a]indol-7-amine

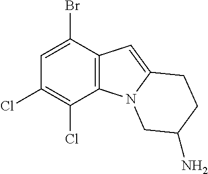 [SMILES: NC1CCc2cc3c(Br)cc(Cl)c(Cl)c3n2C1]

Dissolve 1-bromo-3,4-dichloro-8,9-dihydro-6H-pyrido[1,2-a]indol-7-one (1.35 g, 4.05 mmol) in MeOH (10 mL) and add ammonium acetate (4.69 g, 60.8 mmol), acetic acid (2.44 g, 40.6 mmol) and sodium cyanoborohydride (515 mg, 8.11 mmol). Stir at ambient temperature for 16-18 hrs. Neutralize with saturated aqueous sodium bicarbonate, extract with EtOAc, dry over anhydrous sodium sulfate, filter, and concentrate under vacuum. Purify by trituration with DCM to give 1-bromo-3,4-dichloro-6,7,8,9-tetrahydropyrido[1,2-a]indol-7-amine (1.00 g, 56%) as a gray solid. ES/MS (m/z): 332.9/335.0/337.0 (M+H).

### Intermediate 268

### tert-Butyl N-(1-bromo-3,4-dichloro-6,7,8,9-tetrahydropyrido[1,2-a]indol-7-yl)carbamate

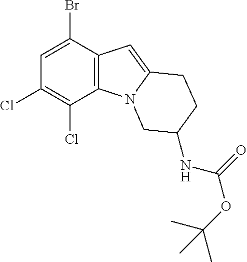 [SMILES: CC(C)(C)OC(=O)NC1CCc2cc3c(Br)cc(Cl)c(Cl)c3n2C1]

Dissolve 1-bromo-3,4-dichloro-6,7,8,9-tetrahydropyrido[1,2-a]indol-7-amine (1.00 g, 2.30 mmol) in DCM (10 mL). Add TEA (1.60 mL, 11.0 mmol) and Boc₂O (1.20 mL, 5.60 mmol) at 0° C. Stir at ambient temperature for 16-18 hrs. Dilute with water, extract with DCM, dry over anhydrous sodium sulfate, filter, and concentrate under vacuum. Purify using column chromatography eluting with EtOAc in petroleum ether to give tert-butyl N-(1-bromo-3,4-dichloro-6,7,8,9-tetrahydropyrido[1,2-a]indol-7-yl)carbamate (600 mg, 58%) as a yellow solid. ES/MS (m/z): 433.0/435.0/437.0 (M+H) and 376.9/378.9/380.9 (M+H-tBu).

### Intermediate 269

### tert-Butyl N-(3,4-dichloro-1-hydroxy-6,7,8,9-tetrahydropyrido[1,2-a]indol-7-yl)carbamate

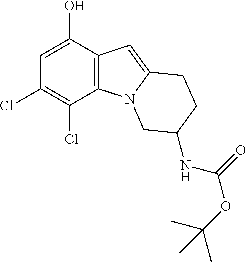 [SMILES: CC(C)(C)OC(=O)NC1CCc2cc3c(O)cc(Cl)c(Cl)c3n2C1]

Suspend tert-butyl N-(1-bromo-3,4-dichloro-6,7,8,9-tetrahydropyrido[1,2-a]indol-7-yl)carbamate (500 mg, 1.09 mmol), CsOH (410 mg, 2.73 mmol) and t-BuBrettPhos-Pd-G₃ (96 mg, 0.11 mmol) in 1,4-dioxane (9 mL) and water (3 mL). Purge with N₂ and heat to 65° C. for 0.5 hr. Cool to ambient temperature, dilute with water, acidify with 1M aqueous HCl to pH=5, extract with EtOAc, dry over anhydrous sodium sulfate, filter, and concentrate under vacuum. Purify using column chromatography eluting with EtOAc in petroleum ether to give tert-butyl N-(3,4-dichloro-1-hydroxy-6,7,8,9-tetrahydropyrido[1,2-a]indol-7-yl)carbamate (200 mg, 40%) as a yellow solid. ES/MS (m/z): (³⁵Cl/³⁷Cl) 371.1/373.0 (M+H) and 315.0/317.1 (M+H-tBu).

### Intermediate 270

### tert-Butyl N-[3,4-dichloro-1-(cyanomethoxy)-6, 7, 8,9-tetrahydropyrido[1,2-a]indol-7-yl]carbamate

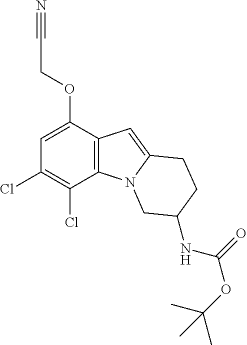 [SMILES: CC(C)(C)OC(=O)NC1CCc2cc3c(OCC#N)cc(Cl)c(Cl)c3n2C1]

Dissolve tert-butyl N-(3,4-dichloro-1-hydroxy-6,7,8,9-tetrahydropyrido[1,2-a]indol-7-yl)carbamate (200 mg, 0.442 mmol) in DMF (5 mL) and add bromoacetonitrile (110 mg, 0.917 mmol) and potassium carbonate (80 mg, 0.58 mmol). Stir at ambient temperature for 2 hrs. Dilute with water, extract with EtOAc, dry over anhydrous sodium sulfate, filter, and concentrate under vacuum. Purify by column chromatography eluting with EtOAc in petroleum ether to give tert-butyl N-[3,4-dichloro-1-(cyanomethoxy)-6,7,8,9-tetrahydropyrido[1,2-a]indol-7-yl]carbamate (170 mg, 79%) as a yellow solid. ES/MS (m/z): (³⁵Cl/³⁷Cl) 410.1/412.0 (M+H) and 354.0/356.0 (M+H-tBu).

### Intermediate 271

### tert-Butyl N-[3,4-dichloro-1-(cyanomethoxy)-10-iodo-6,7,8,9-tetrahydropyrido[1,2-a]indol-7-yl]carbamate

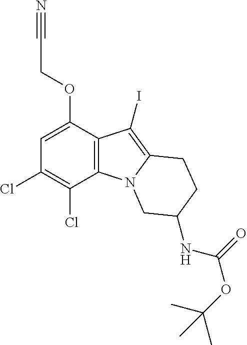 [SMILES: CC(C)(C)OC(=O)NC1CCc2c(I)c3c(OCC#N)cc(Cl)c(Cl)c3n2C1]

Dissolve tert-butyl N-[3,4-dichloro-1-(cyanomethoxy)-6,7,8,9-tetrahydropyrido[1,2-a]indol-7-yl]carbamate (170 mg, 0.348 mmol) in DMF (4 mL) and add NIS (88 mg, 0.383 mmol). Stir at ambient temperature for 1 hr. Dilute with EtOAc, wash with saturated aqueous sodium bicarbonate and saturated aqueous NaCl, dry over anhydrous sodium sulfate, filter, and concentrate under vacuum. Purify by column chromatography eluting with EtOAc in petroleum ether to give tert-butyl N-[3,4-dichloro-1-(cyanomethoxy)-10-iodo-6,7,8,9-tetrahydropyrido[1,2-a]indol-7-yl]carbamate (170 mg, 74%) as a yellow solid. ES/MS (m/z): (³⁵Cl/³⁷Cl) 536.0/537.9 (M+H) and 479.8/481.9 (M+H-tBu).

### Intermediate 272

### tert-Butyl N-[3,4-dichloro-1-(cyanomethoxy)-10-(1-tetrahydropyran-2-ylpyrazol-4-yl)-6,7, 8,9-tetrahydropyrido[1,2-a]indol-7-yl]carbamate

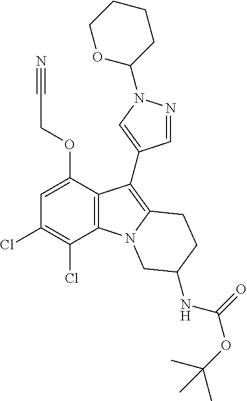 [SMILES: CC(C)(C)OC(=O)NC1CCc2c(-c3cnn(C4CCCCO4)c3)c3c(OCC#N)cc(Cl)c(Cl)c3n2C1]

Suspend tert-butyl N-[3,4-dichloro-1-(cyanomethoxy)-10-iodo-6,7,8,9-tetrahydropyrido[1,2-a]indol-7-yl]carbamate (140 mg, 0.211 mmol), 1-(tetrahydro-2H-pyran-2-yl)-4-(4,4,5,5-tetramethyl-1,3,2-dioxaborolan-2-yl)-1H-pyrazole (90 mg, 0.32 mmol), Pd(dppf)Cl₂ (32 mg, 0.043 mmol) and Na₂CO₃ (68 mg, 0.64 mmol) in 1,4-dioxane (4 mL) and water (0.5 mL). Purge with N₂ and heat to 90° C. for 1 hr. Cool to ambient temperature and concentrate under vacuum. Purify using column chromatography eluting with EtOAc in petroleum ether to give tert-butyl N-[3,4-dichloro-1-(cyanomethoxy)-10-(1-tetrahydropyran-2-ylpyrazol-4-yl)-6,7,8,9-tetrahydropyrido[1,2-a]indol-7-yl]carbamate (100 mg, 61%) as a yellow solid. ES/MS (m/z): (³⁵Cl/³⁷Cl) 560.2/562.2 (M+H) and 476.2/478.2 (M+H-THP).

### Intermediate 273

### 2-[[7-Amino-3,4-dichloro-10-(1H-pyrazol-4-yl)-6,7,8,9-tetrahydropyrido[1,2-a]indol-1-yl]oxy]acetonitrile bis(2,2,2-trifluoroacetate)

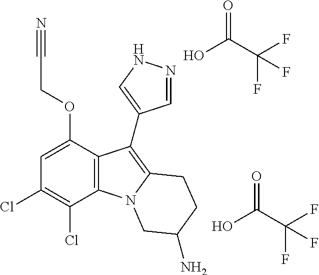

Dissolve tert-butyl N-[3,4-dichloro-1-(cyanomethoxy)-10-(1-tetrahydropyran-2-ylpyrazol-4-yl)-6,7,8,9-tetrahydropyrido[1,2-a]indol-7-yl]carbamate (70 mg, 0.091 mmol) in DCM (2 mL) and add TFA (1 mL). Stir at ambient temperature for 1 hr. Concentrate to give 2-[[7-amino-3,4-dichloro-10-(1H-pyrazol-4-yl)-6,7,8,9-tetrahydropyrido[1,2-a]indol-1-yl]oxy]acetonitrile bis(2,2,2-trifluoroacetate) (70 mg, 48%, TFA salt) as a yellow solid. ES/MS (m/z): (³⁵Cl/³⁷Cl) 376.0/378.0 (M+H).

### EXAMPLE 102

### N-[3,4-Dichloro-1-(cyanomethoxy)-10-(1H-pyrazol-4-yl)-6,7,8,9-tetrahydropyrido[1,2-a]indol-7-yl]acetamide

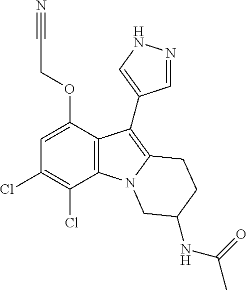 [SMILES: CC(=O)NC1CCc2c(-c3cn[nH]c3)c3c(OCC#N)cc(Cl)c(Cl)c3n2C1]

Dissolve 2-[[7-amino-3,4-dichloro-10-(1H-pyrazol-4-yl)-6,7,8,9-tetrahydropyrido[1,2-a]indol-1-yl]oxy]acetonitrile bis(2,2,2-trifluoroacetate) (70 mg, 0.044 mmol) and TEA (0.05 mL, 0.4 mmol) in DCM (2 mL). Add acetic anhydride (0.010 mL, 0.11 mmol) at 0° C. and stir at 0° C. for 2 hrs. Concentrate under vacuum and dissolve the residue in THF (3 mL) and water (1 mL). Add LiOH (5 mg, 0.21 mmol) at 0° C. Stir at 0° C. for 0.5 hr. Neutralize with 1M aqueous HCl, dilute with water, extract with EtOAc, dry over anhydrous sodium sulfate, filter, and concentrate under vacuum. Purify by prep-HPLC to give N-[3,4-dichloro-1-(cyanomethoxy)-10-(1H-pyrazol-4-yl)-6,7,8,9-tetrahydropyrido[1,2-a]indol-7-yl]acetamide (2.52 mg, 13%) as an off-white solid. ES/MS (m/z): (³⁵Cl/³⁷Cl) 418.3/420.3 (M+H).

### Intermediate 274

### N-((6, 7-Dichloro-3-(1-(tetrahydro-2H-pyran-2-yl)-1H-pyrazol-4-yl)-1H-indol-2-yl)methyl)-2-(4H-1,2,4-triazol -4-yl)acetamide

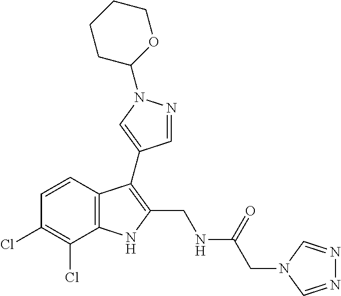 [SMILES: O=C(Cn1cnnc1)NCc1[nH]c2c(Cl)c(Cl)ccc2c1-c1cnn(C2CCCCO2)c1]

Dissolve [6,7-Dichloro-3-(1-tetrahydropyran-2-ylpyrazol-4-yl)-1H-indol-2-yl]methanamine (100 mg, 0.219 mmol) and 2-(1,2,4-triazol-4-yl) acetic acid (37 mg, 0.26 mmol) in DMF (2 mL). Add HATU (102 mg, 0.263 mmol) and DIPEA (283 mg, 2.19 mmol). Stir at ambient temperature for 16-18 hrs. Concentrate under vacuum and purify by column chromatography eluting with DCM/MeOH to give N-((6,7-dichloro-3-(1-(tetrahydro-2H-pyran-2-yl)-1H-pyrazol-4-yl)-1H-indol-2-yl)methyl)-2-(4H-1,2,4-triazol-4-yl)acetamide (37 mg, 32%) as a white solid. ES/MS (m/z): (³⁵Cl/³⁷Cl) 474.1/476.1 (M+H) and 390.0/392.0 (M+H-THP).

### EXAMPLE 103

### N-((6, 7-Dichloro-3-(1H-pyrazol-4-yl)-1H-indol-2-yl)methyl)-2-(4H-1,2,4-triazol -4-yl)acetamide

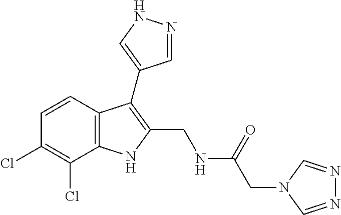 [SMILES: O=C(Cn1cnnc1)NCc1[nH]c2c(Cl)c(Cl)ccc2c1-c1cn[nH]c1]

Dissolve N-((6, 7-di chloro-3-(1-(tetrahydro-2H-pyran-2-yl)-1H-pyrazol -4-yl)-1H-indol-2-yl)methyl)-2-(4H-1,2,4-triazol -4-yl)acetamide (37 mg, 0.070 mmol) in DCM (1 mL). Add TFA (1 mL) and stir at 20° C. for 2 hrs. Concentrate under vacuum and purify by prep-HPLC to give N-((6,7-dichloro-3-(1H-pyrazol-4-yl)-1H-indol-2-yl)methyl)-2-(4H-1,2,4-triazol-4-yl)acetamide (9.55 mg, 34%) as a white solid. ES/MS (m/z): (³⁵Cl/³⁷Cl) 390.3/392.3 (M+H).

### Intermediate 275

### N-((6, 7-Dichloro-3-(1-(tetrahydro-2H-pyran-2-yl)-1H-pyrazol-4-yl)-1H-indol-2-yl)methyl)thiazole-4-carboxamide

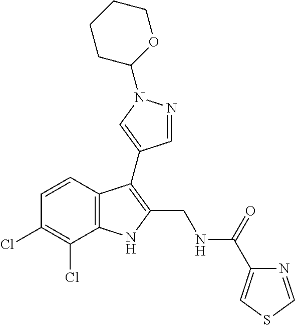 [SMILES: O=C(NCc1[nH]c2c(Cl)c(Cl)ccc2c1-c1cnn(C2CCCCO2)c1)c1cscn1]

Dissolve [6,7-Dichloro-3-(1-tetrahydropyran-2-ylpyrazol-4-yl)-1H-indol-2-yl]methanamine (104 mg, 0.242 mmol) and thiazole-4-carboxylic acid (38 mg, 0.29 mmol) in DMF (2 mL). Add HATU (113 mg, 0.291 mmol) and DIPEA (156 mg, 1.21 mmol). Stir at ambient temperature for 16-18 hrs. Concentrate under vacuum and purify by column chromatography eluting with DCM/MeOH to give N-((6,7-dichloro-3-(1-(tetrahydro-2H-pyran-2-yl)-1H-pyrazol-4-yl)-1H-indol-2-yl)methyl)thiazole-4-carboxamide (129 mg, 90%) as a yellow gum. ES/MS (m/z): (³⁵Cl/³⁷Cl) 476.1/478.1 (M+H) and 392.0/393.9 (M+H-THP).

### EXAMPLE 104

### N-((6,7-Dichloro-3-(1H-pyrazol-4-yl)-1H-indol-2-yl)methyl)thiazole-4-carboxamide

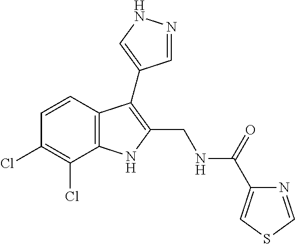 [SMILES: O=C(NCc1[nH]c2c(Cl)c(Cl)ccc2c1-c1cn[nH]c1)c1cscn1]

Dissolve N-((6,7-dichloro-3-(1-(tetrahydro-2H-pyran-2-yl)-1H-pyrazol-4-yl)-1H-indol-2-yl)methyl)thiazole-4-carboxamide (129 mg, 0.217 mmol) in DCM (1 mL). Add TFA (1 mL) and stir at 20° C. for 1 hr. Concentrate under vacuum and purify by reverse phase prep-HPLC to give N-((6,7-dichloro-3-(1H-pyrazol-4-yl)-1H-indol-2-yl)methyl)thiazole-4-carboxamide (25.41 mg 30%) as a white solid. ES/MS (m/z): (³⁵Cl/³⁷Cl) 392.2/394.2 (M+H).

### Intermediate 276

### N-((6,7-Dichloro-3-(1-(tetrahydro-2H-pyran-2-yl)-1H-pyrazol-4-yl)-1H-indol-2-yl)methyl)ethanesulfonamide

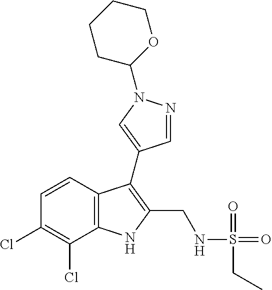 [SMILES: CCS(=O)(=O)NCc1[nH]c2c(Cl)c(Cl)ccc2c1-c1cnn(C2CCCCO2)c1]

Dissolve [6,7-dichloro-3-(1-tetrahydropyran-2-ylpyrazol-4-yl)-1H-indol-2-yl]methanamine (110 mg, 0.256 mmol) and DIPEA (166 mg, 1.28 mmol) in DMF (2 mL). Add ethanesulfonyl chloride (40 mg, 0.31 mmol). Stir at ambient temperature for 16-18 hrs. Concentrate under vacuum and purify by column chromatography eluting with DCM/MeOH to give N-((6,7-dichloro-3-(1-(tetrahydro-2H-pyran-2-yl)-1H-pyrazol-4-yl)-1H-indol-2-yl)methyl)ethanesulfonamide (129 mg, 88%) as a red gum. ES/MS (m/z): (³⁵Cl/³⁷Cl) 457.1/459.1 (M+H) and 373.0/375.0 (M+H-THP).

### EXAMPLE 105

### N-((6,7-Dichloro-3-(1H-pyrazol-4-yl)-1H-indol-2-yl)methyl)ethanesulfonamide

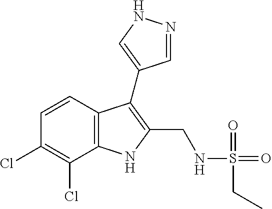 [SMILES: CCS(=O)(=O)NCc1[nH]c2c(Cl)c(Cl)ccc2c1-c1cn[nH]c1]

Dissolve N-((6,7-dichloro-3-(1-(tetrahydro-2H-pyran-2-yl)-1H-pyrazol-4-yl)-1H-indol-2-yl)methypethanesulfonamide (142 mg, 0.248 mmol) in DCM (1 mL). Add TFA (1 mL) and stir at 20° C. for 1 hr. Concentrate under vacuum and purify by prep-HPLC to give N-((6,7-dichloro-3-(1H-pyrazol-4-yl)-1H-indol-2-yl)methyl)ethanesulfonamide (10.40 mg, 11%) as a white solid. ES/MS (m/z): (³⁵Cl/³⁷Cl) 373.2/375.2 (M+H).

### Intermediate 277

### 2-Acetamido-N-((6,7-dichloro-3-(1-(tetrahydro-2H-pyran-2-yl)-1H-pyrazol-4-yl)-1H-indol-2-yl)methyl)acetamide

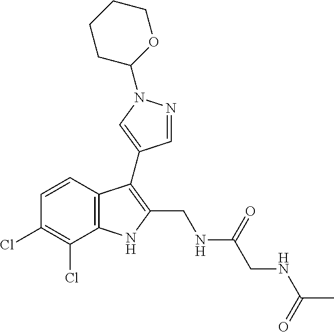 [SMILES: CC(=O)NCC(=O)NCc1[nH]c2c(Cl)c(Cl)ccc2c1-c1cnn(C2CCCCO2)c1]

Dissolve [6,7-dichloro-3-(1-tetrahydropyran-2-ylpyrazol-4-yl)-1H-indol-2-yl]methanamine (129 mg, 0.300 mmol) and N-acetylglycine (43 mg, 0.36 mmol) in DMF (2 mL). Add HATU (140 mg, 0.361 mmol) and DIPEA (194 mg, 1.50 mmol). Stir at ambient temperature for 16-18 hrs. Concentrate under vacuum and purify by column chromatography eluting with DCM/MeOH to give 2-acetamido-N-((6,7-dichloro-3-(1-(tetrahydro-2H-pyran-2-yl)-1H-pyrazol-4-yl)-1H-indol-2-yl)methyl)acetamide (49 mg, 30%) as a white solid. ES/MS (m/z): (³⁵Cl/³⁷Cl) 464.3/466.1 (M+H).

### EXAMPLE 106

### 2-Acetamido-N-((6,7-dichloro-3-(1H-pyrazol-4-yl)-1H-indol-2-yl)methyl)acetamide

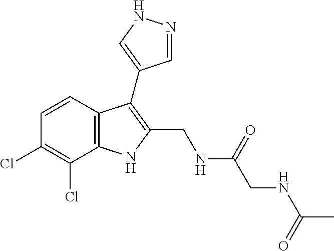 [SMILES: CC(=O)NCC(=O)NCc1[nH]c2c(Cl)c(Cl)ccc2c1-c1cn[nH]c1]

Dissolve 2-acetamido-N-((6,7-dichloro-3-(1-(tetrahydro-2H-pyran-2-yl)-1H-pyrazol-4-yl)-1H-indol-2-yl)methyl)acetamide (49 mg, 0.084 mmol) in DCM (1 mL). Add TFA (1 mL) and stir at 20° C. for 1 hr. Concentrate under vacuum and purify by prep-HPLC to give 2-acetamido-N-((6,7-dichloro-3-(1H-pyrazol-4-yl)-1H-indol-2-yl)methyl)acetamide (19.37 mg, 60%) as a white solid. ES/MS (m/z): (³⁵Cl/³⁷Cl) 380.2/382.2 (M+H).

### Intermediate 278

### 2-Acetamido-N-[3,4-dichloro-10-(1-tetrahydropyran-2-ylpyrazol-4-yl)-6,7,8,9-tetrahydropyrido[1,2-a]indol-7-yl]acetamide

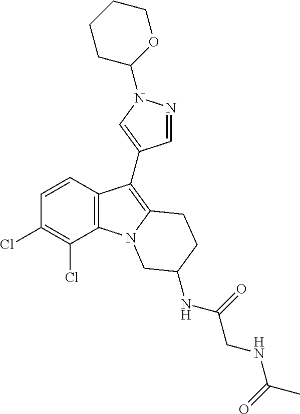 [SMILES: CC(=O)NCC(=O)NC1CCc2c(-c3cnn(C4CCCCO4)c3)c3ccc(Cl)c(Cl)c3n2C1]

Suspend 3,4-dichloro-10-(1-tetrahydropyran-2-ylpyrazol-4-yl)-6,7,8,9-tetrahydropyrido[1,2-a]indol-7-amine (180 mg, 0.422 mmol), N-acetylglycine (105 mg, 0.852 mmol) and DIPEA (0.368 mL, 2.11 mmol) in DMF (4 mL) and add HATU (330 mg, 0.851 mmol). Stir at ambient temperature for 2 hrs. Dilute with water, extract with EtOAc, dry over anhydrous sodium sulfate, filter, and concentrate under vacuum. Purify by column chromatography eluting with MeOH in DCM to give 2-acetamido-N-[3,4-dichloro-10-(1-tetrahydropyran-2-ylpyrazol-4-yl)-6,7,8,9-tetrahydropyrido[1,2-a]indol-7-yl]acetamide (180 mg, 76%) as a white solid. ES/MS (m/z): (³⁵Cl/³⁷Cl) 504.2/506.2 (M+H) and 420.1/422.1 (M+H-THP).

### EXAMPLE 107

### 2-Acetamido-N-[3,4-dichloro-10-(1H-pyrazol-4-yl)-6, 7,8, 9-tetrahydropyrido[1,2-a]indol-7-yl]acetamide

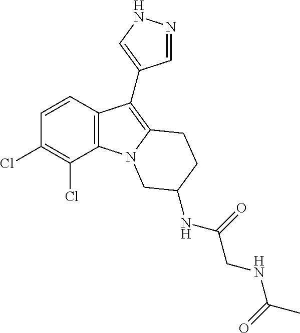 [SMILES: CC(=O)NCC(=O)NC1CCc2c(-c3cn[nH]c3)c3ccc(Cl)c(Cl)c3n2C1]

Dissolve 2-acetamido-N-[3,4-dichloro-10-(1-tetrahydropyran-2-ylpyrazol-4-yl)-6,7,8,9-tetrahydropyrido[1,2 a]indol-7-yl]acetamide (180 mg, 0.321 mmol) in DCM (3 mL) and add TFA (3 mL). Stir at ambient temperature for 3 hrs. Combine with another batch run at 0.59×scale for work up and purification. Concentrate under vacuum and purify by prep-HPLC to give 2-acetamido-N-[3,4-dichloro-10-(1H-pyrazol-4-yl)-6,7,8,9-tetrahydropyrido[1,2-a]indol-7-yl]acetamide (86.30 mg, 40%) as a white solid. ES/MS (m/z): (³⁵Cl/³⁷Cl) 420.3/422.3 (M+H).

### EXAMPLE 108

### 1-[6,7-Dichloro-10-(1H-pyrazol-4-yl)-3,4-dihydro-1H-pyrazino[1,2-a]indol-2-yl]-2,4-dimethoxy-butan-1-one

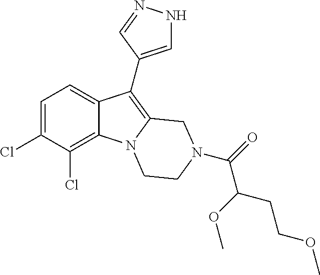 [SMILES: COCCC(OC)C(=O)N1CCn2c(c(-c3cn[nH]c3)c3ccc(Cl)c(Cl)c32)C1]

Suspend 6,7-dichloro-10-(1H-pyrazol-4-yl)-1,2,3,4-tetrahydropyrazino[1,2-a]indole (50 mg, 0.14 mmol, HCl salt) in DMF (1 mL). Add DIPEA (0.2 mL) and stir at ambient temperature for 15 min. Add 2,4-dimethoxybutanoic acid (CAS: 1832579-54-0, 35 mg, 0.21 mmol) and HATU (65 mg, 0.17 mmol). Stir at ambient temperature for 16-18 hrs. Filter and concentrate under vacuum. Purify by prep-HPLC to give 1-[6,7-dichloro-10-(1H-pyrazol-4-yl)-3,4-dihydro-1H-pyrazino[1,2-a]indol-2-yl]-2,4-dimethoxy-butan-1-one (18.48 mg, 31%) as a white solid. ES/MS (m/z): (³⁵Cl/³⁷Cl) 437.3/439.3 (M+H).

### Intermediate 279

### tert-Butyl 3-(6,7-dichloro-10-(1H-pyrazol-4-yl)-1,2,3,4-tetrahydropyrazino[1,2-a]indole-2-carbonyl)morpholine-4-carboxylate

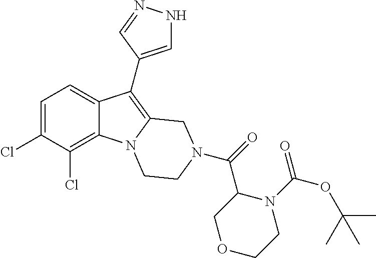 [SMILES: CC(C)(C)OC(=O)N1CCOCC1C(=O)N1CCn2c(c(-c3cn[nH]c3)c3ccc(Cl)c(Cl)c32)C1]

Dissolve 4-boc-3-morpholinecarboxylic acid (133 mg, 0.558 mmol), HATU (218 mg, 0.556 mmol) and DIPEA (184 mg, 1.40 mmol) in DMF (3 mL). Add a solution of 6,7-dichloro-10-(1H-pyrazol-4-yl)-1,2,3,4-tetrahydropyrazino[1,2-a]indole (150 mg, 0.393 mmol, HCl salt) in DMF (3 mL). Stir at ambient temperature for 16-18 hrs. Dilute with water and extract with EtOAc. Wash with 10% aqueous LiCl and saturated aqueous NaCl, dry over anhydrous sodium sulfate, filter, and concentrate under vacuum to give tert-butyl 3-(6,7-dichloro-10-(1H-pyrazol-4-yl)-1,2,3,4-tetrahydropyrazino[1,2-a]indole-2-carbonyl)morpholine-4-carboxylate (192 mg, 75%) as a yellow solid. ES/MS (m/z): (³⁵Cl/³⁷Cl) 520.1/522.3 (M+H) and 420.2/422.1 (M+H-Boc).

### Intermediate 280

### (6,7-Dichloro-10-(1H-pyrazol-4-yl)-3,4-dihydropyrazino[1,2-a]indol-2(1H)-yl)(morpholin-3-yl)methanone

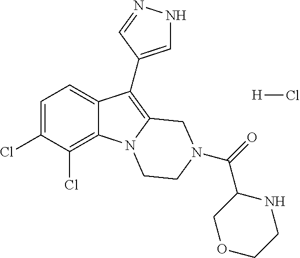

Dissolve tert-butyl 3-(6,7-dichloro-10-(1H-pyrazol-4-yl)-1,2,3,4-tetrahydropyrazino[1,2-a]indole-2-carbonyl)morpholine-4-carboxylate (192 mg, 0.295 mmol) in 4M HCl/dioxane (5 mL). Stir at ambient temperature for 1 hr. Concentrate and dry under vacuum to give (6,7-dichloro-10-(1H-pyrazol-4-yl)-3,4-dihydropyrazino[1,2-a]indol-2(1H)-yl)(morpholin-3-yl)methanone (120 mg, HCl salt, 91%) as a yellow solid. ES/MS (m/z): (³⁵Cl/³⁷Cl) 420.0/422.0 (M+H).

### EXAMPLE 109

### 1-(3-(6,7-Dichloro-10-(1H-pyrazol-4-yl)-1,2,3,4-tetrahydropyrazino[1,2-a]indole-2-carbonyl)morpholino)ethan-1-one

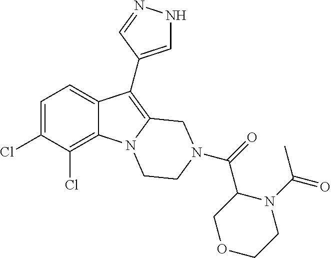 [SMILES: CC(=O)N1CCOCC1C(=O)N1CCn2c(c(-c3cn[nH]c3)c3ccc(Cl)c(Cl)c32)C1]

Dissolve (6,7-dichloro-10-(1H-pyrazol-4-yl)-3,4-dihydropyrazino[1,2-a]indol-2(1H)-yl)(morpholin-3-yl)methanone (120 mg, 0.271 mmol, HCl salt) and TEA (0.2 mL, 1 mmol) in DCM (2 mL). Add acetic anhydride (42 mg, 0.41 mmol) at 0° C. Warm to ambient temperature and stir 2 hrs. Concentrate under vacuum and dissolve in MeOH (5 mL). Add K₂CO₃ (113 mg, 0.818 mmol) and stir at ambient temperature for another 2 hrs. Concentrate under vacuum and purify by prep-HPLC to give 1-(3-(6,7-dichloro-10-(1H-pyrazol-4-yl)-1,2,3,4-tetrahydropyrazino[1,2-a]indole-2-carbonyl)morpholino)ethan-1-one (5.33 mg, 4%) as a white solid. ES/MS (m/z): (³⁵Cl/³⁷Cl) 462.3/464.3 (M+H) and 484.3/486.2 (M+Na).

### EXAMPLE 110

### 1-[6,7-Dichloro-10-(1H-pyrazol-4-yl)-3,4-dihydro-1H-pyrazino[1,2-a]indol-2-yl]-3-(2-methyl-1,2,4-triazol-3-yl)propan-1-one

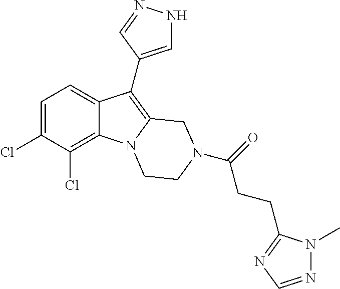 [SMILES: Cn1ncnc1CCC(=O)N1CCn2c(c(-c3cn[nH]c3)c3ccc(Cl)c(Cl)c32)C1]

Suspend 6,7-dichloro-10-(1H-pyrazol-4-yl)-1,2,3,4-tetrahydropyrazino[1,2-a]indole (50 mg, 0.14 mmol, HCl salt) in DMF (1 mL). Add DIPEA (0.2 mL) and stir at ambient temperature for 15 min. Then add 3-(2-methyl-1,2,4-triazol-3-yl)propanoic acid (CAS: 1247443-88-4, 113 mg, 0.655 mmol) and HATU (65 mg, 0.17 mmol). Stir at ambient temperature for 16-18 hrs. Purify by prep-HPLC to give 1-[6,7-dichloro-10-(1H-pyrazol-4-yl)-3,4-dihydro-1H-pyrazino[1,2-a]indol-2-yl]-3-(2-methyl-1,2,4-triazol-3-yl)propan-1-one (18.4 mg, 30%) as a white solid. ES/MS (m/z): (³⁵Cl/³⁷Cl) 444.3/446.3 (M+H).

### EXAMPLE 111

### 1-[6,7-Dichloro-10-(1H-pyrazol-4-yl)-3,4-dihydro-1H-pyrazino[1,2-a]indol-2-yl]-2-imidazol-1-yl-ethanone

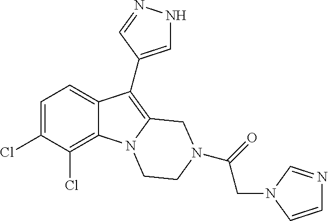 [SMILES: O=C(Cn1ccnc1)N1CCn2c(c(-c3cn[nH]c3)c3ccc(Cl)c(Cl)c32)C1]

Suspend 6,7-dichloro-10-(1H-pyrazol-4-yl)-1,2,3,4-tetrahydropyrazino[1,2-a]indole hydrochloride (50 mg, 0.14 mmol) in DMF (1 mL). Add DIPEA (0.2 mL) and stir at ambient temperature for 15 min. Add 2-(1H-imidazol-1-yl) acetic acid (27 mg, 0.21 mmol) and HATU (65 mg, 0.17 mmol). Stir at ambient temperature for 16-18 hrs. Filter and concentrate under vacuum. Purify by prep-HPLC to give 1-[6,7-dichloro-10-(1H-pyrazol-4-yl)-3,4-dihydro-1H-pyrazino[1,2-a]indol-2-yl]-2-imidazol-1-yl-ethanone (10.9 mg, 18%) as a yellow solid. ES/MS (m/z): (³⁵Cl/³⁷Cl) 415.3/417.3 (M+H).

### Intermediate 281

### N-((6,7-Dichloro-3-(1-(tetrahydro-2H-pyran-2-yl)-1H-pyrazol-4-yl)-1H-indol-2-yl)methyl)-2-(pyrazin-2-yl)acetamide

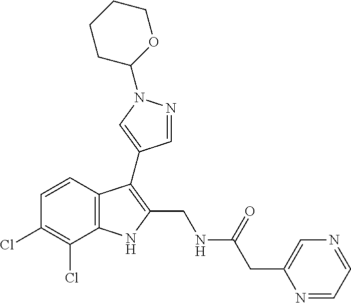 [SMILES: O=C(Cc1cnccn1)NCc1[nH]c2c(Cl)c(Cl)ccc2c1-c1cnn(C2CCCCO2)c1]

Dissolve (6, 7-dichloro-3-(1-(tetrahydro-2H-pyran-2-yl)-1H-pyrazol -4-yl)-1H-indol-2-yl) methanamine (139 mg, 0.285 mmol) and 2-pyrazin-2-ylacetic acid (47 mg, 0.34 mmol) in DMF (2 mL). Add HATU (133 mg, 0.342 mmol) and DIPEA (184 mg, 1.42 mmol) and stir at ambient temperature for 16-18 hrs. Concentrate under vacuum and purify by flash column chromatography eluting with DCM/MeOH to give N-((6,7-dichloro-3-(1-(tetrahydro-2H-pyran-2-yl)-1H-pyrazol-4-yl)-1H-indol-2-yl)methyl)-2-(pyrazin-2-yl)acetamide (146 mg, 95%) as a red gum. ES/MS (m/z): (³⁵Cl/³⁷Cl) 485.1/487.1 (M+H).

### EXAMPLE 112

### N-((6,7-Dichloro-3-(1H-pyrazol-4-yl)-1H-indol-2-yl)methyl)-2-(pyrazin-2-yl)acetamide

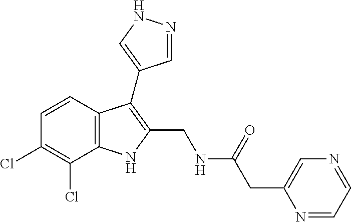 [SMILES: O=C(Cc1cnccn1)NCc1[nH]c2c(Cl)c(Cl)ccc2c1-c1cn[nH]c1]

Dissolve N-((6,7-dichloro-3-(1-(tetrahydro-2H-pyran-2-yl)-1H-pyrazol-4-yl)-1H-indol-2-yl)methyl)-2-(pyrazin-2-yl)acetamide (100 mg, 0.185 mmol) in THF (1 mL). Add 6M aqueous HCl (2 mL) and stir at ambient temperature for 1 hr. Concentrate under vacuum and purify by prep-HPLC to give N-((6,7-dichloro-3-(1H-pyrazol-4-yl)-1H-indol-2-yl)methyl)-2-(pyrazin-2-yl)acetamide (13.55 mg, 18%) as a yellow solid. ES/MS (m/z): (³⁵Cl/³⁷Cl) 401.2/403.2 (M+H).

### EXAMPLE 113

### 4-[6,7-Dichloro-9-(cyanomethoxy)-10-(1H-pyrazol-4-yl)-3,4-dihydro-1H-pyrazino[1,2-a]indol-2-yl]-N,N-dimethyl-4-oxo-butanamide

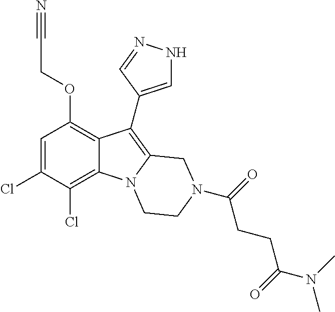 [SMILES: CN(C)C(=O)CCC(=O)N1CCn2c(c(-c3cn[nH]c3)c3c(OCC#N)cc(Cl)c(Cl)c32)C1]

Suspend 2-[[6,7-dichloro-10-(1H-pyrazol-4-yl)-1,2,3,4-tetrahydropyrazino[1,2-a]indol-9-yl]oy]acetonitrile (50 mg, 0.12 mmol, HCl salt) in DMF (1 mL). Add DIPEA (0.2 mL) and stir at ambient temperature for 15 min. Add N,N-dimethylsuccinamic acid (20 mg, 0.14 mmol) and HATU (58 mg, 0.15 mmol). Stir at ambient temperature for 16-18 hrs. Filter and concentrate under vacuum. Purify by prep-HPLC to give 4-[6,7-dichloro-9-(cyanomethoxy)-10-(1H-pyrazol-4-yl)-3,4-dihydro-1H-pyrazino[1,2-a]indol-2-yl]-N,N-dimethyl-4-oxo-butanami de (20.29 mg, 33%) as a yellow solid. ES/MS (m/z): (³⁵Cl/³⁷Cl) 489.3/491.3 (M+H).

### Intermediate 282

### tert-Butyl (S)-3-(2-(6,7-dichloro-9-(cyanomethoxy)-10-(1H-pyrazol-4-yl)-3,4-dihydropyrazino[1,2-a]indol-2(1H)-yl)-2-oxoethyl)morpholine-4-carboxylate

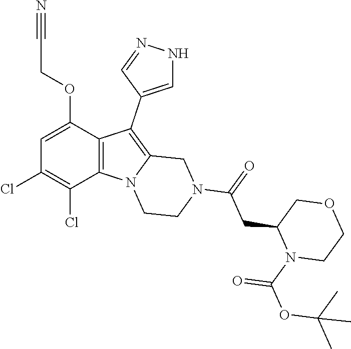 [SMILES: CC(C)(C)OC(=O)N1CCOC[C@@H]1CC(=O)N1CCn2c(c(-c3cn[nH]c3)c3c(OCC#N)cc(Cl)c(Cl)c32)C1]

Dissolve 2-[[6,7-dichloro-10-(1H-pyrazol-4-yl)-1,2,3,4-tetrahydropyrazino[1,2-a]indol-9-yl]oxy] acetonitrile (100 mg, 0.243 mmol, HCl salt), 2-[(3S)-4-tert-butoxycarbonylmorpholin-3-yl]acetic acid (63 mg, 0.26 mmol) and DIPEA (0.13 mL, 0.75 mmol) in DMF (3 mL). Add HATU (120 mg, 0.309 mmol). Stir at ambient temperature for 1 hr. Dilute with water, extract with EtOAc, wash with saturated aqueous NaCl, dry over anhydrous sodium sulfate, filter, and concentrate under vacuum. Dilute the residue with water (1 mL) and THF (2 mL), add LiOH (60 mg, 2.5 mmol) at 0° C., stir at 0° C. for 1 hr. Neutralize with 1M aqueous HCl and dilute with water, extract with EtOAc, dry over anhydrous sodium sulfate, filter, and concentrate under vacuum. Purify by flash column eluting with MeOH in DCM to give tert-butyl (S)-3-(2-(6,7-dichloro-9-(cyanomethoxy)-10-(1H-pyrazol-4-yl)-3,4-dihydropyrazino[1,2-a]indol-2(1H)-yl)-2-oxoethyl)morpholine-4-carboxylate (120 mg, 77%) as yellow oil. ES/MS (m/z): (³⁵Cl/³⁷Cl) 589.1/591.1 (M+H) and 489.1/491.1 (M+H-Boc).

### Intermediate 283

### (S)-2-((6,7-Dichloro-2-(2-(morpholin-3-yl)acetyl)-10-(1H-pyrazol-4-yl)-1,2,3,4-tetrahydropyrazino[1,2-a]indol-9-yl)oxy)acetonitrile

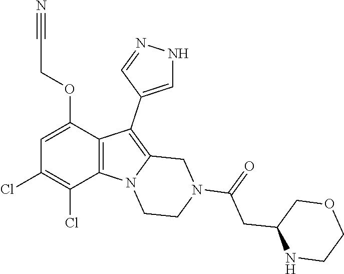 [SMILES: N#CCOc1cc(Cl)c(Cl)c2c1c(-c1cn[nH]c1)c1n2CCN(C(=O)C[C@H]2COCCN2)C1]

Dissolve tert-butyl (S)-3-(2-(6,7-dichloro-9-(cyanomethoxy)-10-(1H-pyrazol-4-yl)-3,4-dihydropyrazino[1,2-a]indol-2(1H)-yl)-2-oxoethyl)morpholine-4-carboxylate (120 mg, 0.187 mmol) in DCM (3 mL). Add TFA (1.5 mL) and stir at ambient temperature for 2 hrs. Concentrate under vacuum to give (S)-2-((6,7-dichloro-2-(2-(morpholin-3-yl)acetyl)-10-(1H-pyrazol-4-yl)-1,2,3,4-tetrahydropyrazino[1,2-a]indol-9-yl)oxy)acetonitrile (130 mg, TFA salt, 94%) as a yellow solid. ES/MS (m/z): (³⁵Cl/³⁷Cl) 489.1/491.1 (M+H).

### EXAMPLE 114

### (S)-2-((6,7-Dichloro-2-(2-(4-methylmorpholin-3-yl)acetyl)-10-(1H-pyrazol-4-yl)-1,2,3,4-tetrahydropyrazino[1,2-a]indol-9-yl)oxy)acetonitrile

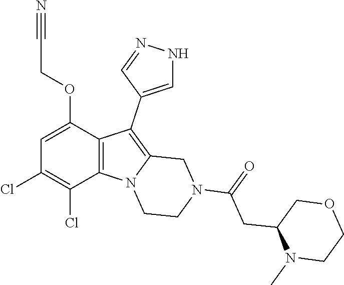 [SMILES: CN1CCOC[C@@H]1CC(=O)N1CCn2c(c(-c3cn[nH]c3)c3c(OCC#N)cc(Cl)c(Cl)c32)C1]

Dissolve (S)-2-((6,7-dichloro-2-(2-(morpholin-3-yl)acetyl)-10-(1H-pyrazol-4-yl)-1,2,3,4-tetrahydropyrazino[1,2-a]indol-9-yl)oxy)acetonitrile (120 mg, 0.163 mmol, TFA salt) in DCE (3 mL). Adjust to pH=8 with DIPEA (0.1 mL, 0.6 mmol), then add acetic acid (0.03 mL, 0.05 mmol) to adjust pH=5. Add formaldehyde (110 mg, 1.36 mmol, 37% in water) and stir at ambient temperature for 0.5 hr. Add sodium cyanoborohydride (35 mg, 0.56 mmol) and stir at ambient temperature for 1 hr. Dilute with water, neutralize with saturated aqueous sodium bicarbonate and extract with DCM, dry over anhydrous sodium sulfate, filter, and concentrate under vacuum. Purify by prep-HPLC to give (S)-2-((6,7-dichloro-2-(2-(4-methylmorpholin-3-yl)acetyl)-10-(1H-pyrazol-4-yl)-1,2,3,4-tetrahydropyrazino[1,2-a]indol-9-yl)oxy)acetonitrile (19.4 mg, 24%) as a white solid. ES/MS (m/z): (³⁵Cl/³⁷Cl) 503.3/505.3 (M+H).

### Intermediate 284

### tert-Butyl (R)-3-(2-(6,7-dichloro-9-(cyanomethoxy)-10-(1H-pyrazol-4-yl)-3,4-dihydropyrazino[1,2-a]indol-2(1H)-yl)-2-oxoethyl)morpholine-4-carboxylate

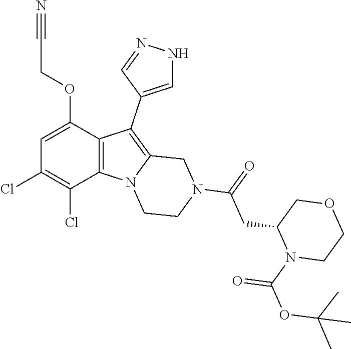 [SMILES: CC(C)(C)OC(=O)N1CCOC[C@H]1CC(=O)N1CCn2c(c(-c3cn[nH]c3)c3c(OCC#N)cc(Cl)c(Cl)c32)C1]

Dissolve 2-[[6,7-dichloro-10-(1H-pyrazol-4-yl)-1,2,3,4-tetrahydropyrazino[1,2-a]indol-9-yl]oxy] acetonitrile (100 mg, 0.243 mmol, HCl salt), 2-[(3R)-4-tert-butoxycarbonylmorpholin-3-yl]acetic acid (63 mg, 0.26 mmol), DIPEA (0.13 mL, 0.75 mmol) in DMF (3 mL). Add HATU (120 mg, 0.309 mmol) and stir at ambient temperature for 2 hrs. Dilute with water, extract with EtOAc, wash with saturated aqueous NaCl, dry over anhydrous sodium sulfate, filter, and concentrate under vacuum. Dilute the residue with water (1 mL) and THF (2 mL), add LiOH (80 mg, 3.3 mmol) at 0° C. and stir at 0° C. for 1 hr. Neutralize with 1M aqueous HCl and extract with EtOAc, dry over anhydrous sodium sulfate, filter, and concentrate under vacuum. Purify by flash column chromatography eluting with MeOH in DCM to give tert-butyl (R)-3-(2-(6,7-dichloro-9-(cyanomethoxy)-10-(1H-pyrazol-4-yl)-3,4-dihydropyrazino[1,2-a]indol-2(1H)-yl)-2-oxoethyl)morpholine-4-carboxylate (100 mg, 66%) as a yellow solid. ES/MS (m/z): (³⁵Cl/³⁷Cl) 489.1/491.1 (M+H-Boc).

### Intermediate 285

### (R)-2-((6,7-Dichloro-2-(2-(morpholin-3-yl)acetyl)-10-(1H-pyrazol-4-yl)-1,2,3,4-tetrahydropyrazino[1,2-a]indol-9-yl)oxy)acetonitrile

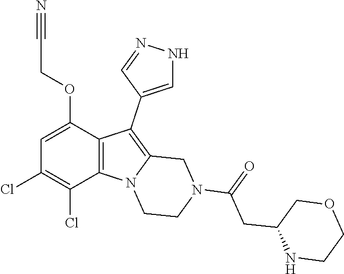 [SMILES: N#CCOc1cc(Cl)c(Cl)c2c1c(-c1cn[nH]c1)c1n2CCN(C(=O)C[C@@H]2COCCN2)C1]

Dissolve tert-butyl (R)-3-(2-(6,7-dichloro-9-(cyanomethoxy)-10-(1H-pyrazol-4-yl)-3,4-dihydropyrazino[1,2-a]indol-2(1H)-yl)-2-oxoethyl)morpholine-4-carboxylate (100 mg, 0.160 mmol) in DCM (3 mL). Add TFA (1.5 mL) and stir at ambient temperature for 2 hr then concentrate under vacuum to give (R)-2-((6,7-dichloro-2-(2-(morpholin-3-yl)acetyl)-10-(1H-pyrazol-4-yl)-1,2,3,4-tetrahydropyrazino[1,2-a]indol-9-yl)oxy)acetonitrile (110 mg, TFA salt, 96%) as a yellow solid. ES/MS (m/z): (³⁵Cl/³⁷Cl) 489.1/491.1 (M+H).

### EXAMPLE 115

### (R)-2-((6,7-Dichloro-2-(2-(4-methylmorpholin-3-yl)acetyl)-10-(1H-pyrazol-4-yl)-1,2,3,4-tetrahydropyrazino[1,2-a]indol-9-yl)oxy)acetonitrile

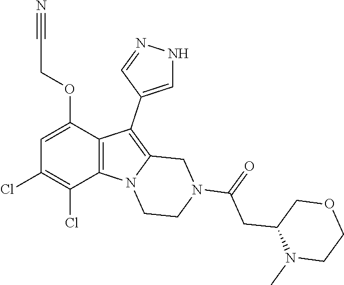 [SMILES: CN1CCOC[C@H]1CC(=O)N1CCn2c(c(-c3cn[nH]c3)c3c(OCC#N)cc(Cl)c(Cl)c32)C1]

Dissolve (R)-2-((6,7-dichloro-2-(2-(morpholin-3-yl)acetyl)-10-(1H-pyrazol-4-yl)-1,2,3,4-tetrahydropyrazino[1,2-a]indol-9-yl)oxy)acetonitrile (110 mg, 0.153 mmol, TFA salt) in DCE (3 mL), Adjust to pH=8 with DIPEA (0.1 mL, 0.6 mmol), then add acetic acid (0.03 mL, 0.05 mmol) to adjust to pH=5. Add formaldehyde (100 mg, 1.23 mmol, 37% in water) and stir at ambient temperature for 0.5 hr. Add sodium cyanoborohydride (30 mg, 0.48 mmol) and stir at ambient temperature for 1 hr. Dilute with water, neutralize with saturated aqueous sodium bicarbonate and extract with DCM, dry over anhydrous sodium sulfate, filter, and concentrate under vacuum. Purify by prep-HPLC and then SFC to give ((R)-2-((6,7-dichloro-2-(2-(4-methylmorpholin-3-yl)acetyl)-10-(1H-pyrazol-4-yl)-1,2,3,4-tetrahydropyrazino[1,2-a]indol-9-yl)oxy)acetonitrile (12.45 mg, 0.024 mmol, 16%) as a white solid. ES/MS (m/z): (³⁵Cl/³⁷Cl) 503.1/505.1 (M+H).

### Intermediate 286

### Methyl 4-(6,7-dichloroindol-1-yl)butanoate

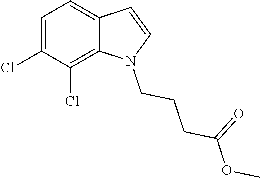 [SMILES: COC(=O)CCCn1ccc2ccc(Cl)c(Cl)c21]

Dissolve 6,7-dichloro-1H-indole (3.00 g, 14.5 mmol) in DMF (40 mL) and add NaH (700 mg, 17.5 mmol, 60% dispersion in mineral oil) slowly at 0° C. Stir at 0° C. for 0.5 hr. Add methyl 4-bromobutyrate (2.3 mL, 18 mmol) at 0° C., stir for 1h, and allow to warm to ambient temperature. Quench with water and extract with DCM (3×). Wash the combined organic layers with saturated aqueous NaCl, dry over anhydrous sodium sulfate, filter, and concentrate under vacuum. Purify by flash column chromatography eluting with EtOAc in petroleum ether to give methyl 4-(6,7-dichloroindol-1-yl)butanoate (2.4 g, 53%) as a light-yellow oil. ES/MS (m/z): (³⁵Cl/³⁷Cl) 286.1/288.1 (M+H).

### Intermediate 287

### 4-(6,7-Dichloroindol-1-yl)butanoic acid

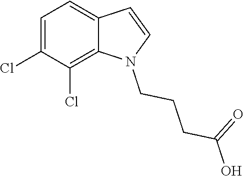 [SMILES: O=C(O)CCCn1ccc2ccc(Cl)c(Cl)c21]

Dissolve methyl 4-(6,7-dichloroindol-1-yl)butanoate (3.28 g, 10.5 mmol) in THF (40 mL) and water (10 mL) and add LiOH (2.65 g, 63.2 mmol). Stir at ambient temperature for 1 hr. Concentrate, dilute with water, and adjust to pH=3 with 1M aqueous HCl. Filter the precipitate, wash with water (3×), and dry under vacuum to give 4-(6,7-dichloroindol-1-yl)butanoic acid (2.76 g, 89%) as an off-white solid. ES/MS (m/z): (³⁵Cl/³⁷Cl) 272.1/274.0 (M+H).

### Intermediate 288

### 3,4-Dichloro-7,8-dihydropyrido[1,2-a]indol-9 (6H)-one

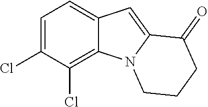 [SMILES: O=C1CCCn2c1cc1ccc(Cl)c(Cl)c12]

Dissolve 4-(6,7-dichloroindol-1-yl)butanoic acid (2.76 g, 9.35 mmol) in polyphosphoric acid (50 g). Stir at 90° C. for 1 hr. Cool to about 70° C. and quench with ice-water, stir for 0.5 h, and extract with EtOAc (3×). Wash the combined organic layers with saturated aqueous sodium bicarbonate (3×), dry over anhydrous sodium sulfate, filter, and concentrate under vacuum. Purify by flash column chromatography eluting with EtOAc in petroleum ether and triturate with MeOH at 50° C. to give 3,4-dichloro-7,8-dihydropyrido[1,2-a]indol-9(6H)-one (667 mg, 26%) as a yellow solid. ES/MS (m/z): (³⁵Cl/³⁷Cl) 254.1/256.1 (M+H).

### Intermediate 289

### 3,4-Dichloro-6,7,8,9-tetrahydropyrido[1,2-a]indol-9-amine

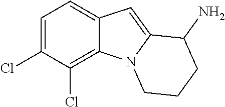 [SMILES: NC1CCCn2c1cc1ccc(Cl)c(Cl)c12]

Suspend 3,4-dichloro-7,8-dihydropyrido[1,2-a]indol-9(6H)-one (350 mg, 1.28 mmol), ammonium acetate (1.48 g, 19.2 mmol) and acetic acid (771 mg, 12.8 mmol) in MeOH (8 mL). Add sodium cyanoborohydride (163 mg, 2.57 mmol) and stir at 50° C. for 16-18 hrs. Neutralize with saturated aqueous sodium bicarbonate, extract with EtOAc, dry over anhydrous sodium sulfate, filter, and concentrate under vacuum. Purify by flash column chromatography eluting with MeOH in DCM to give 3,4-dichloro-6,7,8,9-tetrahydropyrido[1,2-a]indol-9-amine (220 mg, 64%) as a yellow gum.

### Intermediate 290

### N-(3,4-Dichloro-6,7,8,9-tetrahydropyrido[1,2-a]indol-9-yl)acetamide

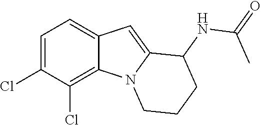 [SMILES: CC(=O)NC1CCCn2c1cc1ccc(Cl)c(Cl)c12]

Dissolve 3,4-dichloro-6,7,8,9-tetrahydropyrido[1,2-a]indol-9-amine (220 mg, 0.819 mmol) and TEA (0.57 mL, 4.09 mmol) in DCM (5 mL). Add Ac₂O (0.10 mL, 1.1 mmol) at 0° C. Stir for 2 hrs. Neutralize with 1M aqueous HCl, dilute with water, extract with EtOAc, dry over anhydrous sodium sulfate, filter, and concentrate under vacuum to give N-(3,4-dichloro-6,7,8,9-tetrahydropyrido[1,2-a]indol-9-yl)acetamide (200 mg, 78%) as a yellow solid.

### Intermediate 291

### N-(3,4-Dichloro-10-iodo-6,7,8,9-tetrahydropyrido[1,2-a]indol-9-yl)acetamide

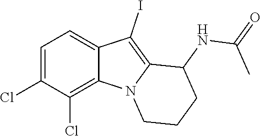 [SMILES: CC(=O)NC1CCCn2c1c(I)c1ccc(Cl)c(Cl)c12]

Dissolve N-(3,4-dichloro-6,7,8,9-tetrahydropyrido[1,2-a]indol-9-yl)acetamide (200 mg, 0.639 mmol) in DMF (5 mL) and add NIS (162 mg, 0.706 mmol). Stir at ambient temperature for 1 hr. Dilute with EtOAc, wash with saturated aqueous sodium bicarbonate and saturated aqueous NaCl, dry over anhydrous sodium sulfate, filter, and concentrate under vacuum to give N-(3,4-dichloro-10-iodo-6,7,8,9-tetrahydropyrido[1,2-a]indol-9-yl)acetamide (230 mg, 81%) as a yellow solid.

### Intermediate 292

### N-[3,4-Dichloro-10-(1-tetrahydropyran-2-ylpyrazol-4-yl)-6, 7,8, 9-tetrahydropyrido[1,2-a]indol-9-yl]acetamide

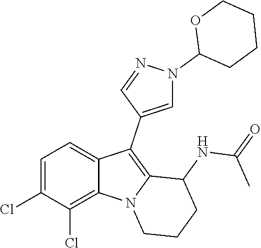 [SMILES: CC(=O)NC1CCCn2c1c(-c1cnn(C3CCCCO3)c1)c1ccc(Cl)c(Cl)c12]

Suspend N-(3,4-dichloro-10-iodo-6,7,8,9-tetrahydropyrido[1,2-a]indol-9-yl)acetamide (200 mg, 0.449 mmol), 1-(tetrahydro-2H-pyran-2-yl)-4-(4,4,5,5-tetramethyl-1,3,2-dioxaborolan-2-yl)-1H-pyrazole (191 mg, 0.673 mmol), Pd(dtbpf)Cl₂ (60 mg, 0.090 mmol) and Na₂CO₃ (144 mg, 1.35 mmol) in 1,4-dioxane (8 mL) and water (1 mL). Purge with N₂ and heat to 90° C. for 2 hrs. Concentrate under vacuum and purify by flash column chromatography eluting with EtOAc in petroleum ether to give N-[3,4-dichloro-10-(1-tetrahydropyran-2-ylpyrazol-4-yl)-6,7,8,9-tetrahydropyrido[1,2-a]indol-9-yl]acetamide (100 mg, 43%) as a yellow solid. ES/MS (m/z): (³⁵Cl/³⁷Cl) 447.1/449.0 (M+H).

### EXAMPLE 116

### N-(3,4-Dichloro-10-(1H-pyrazol-4-yl)-6,7,8,9-tetrahydropyrido[1,2-a]indol-9-yl)acetamide

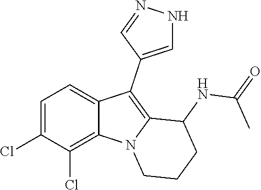 [SMILES: CC(=O)NC1CCCn2c1c(-c1cn[nH]c1)c1ccc(Cl)c(Cl)c12]

Dissolve N-[3,4-dichloro-10-(1-tetrahydropyran-2-ylpyrazol-4-yl)-6,7,8,9-tetrahydropyrido[1,2-a]indol-9-yl]acetamide (100 mg, 0.192 mmol) in DCM (2 mL) and add TFA (2 mL). Stir at ambient temperature for 16-18 hrs. Concentrate under vacuum and purify by prep-HPLC to give N-(3,4-dichloro-10-(1H-pyrazol-4-yl)-6,7,8,9-tetrahydropyrido[1,2-a]indol-9-yl)acetamide (28.77 mg, 40%) as an off-white solid. ES/MS (m/z): (³⁵Cl/³⁷Cl) 363.2/365.3 (M+H).

### Intermediate 293

### N-[3,4-Dichloro-10-(1-tetrahydropyran-2-ylpyrazol-4-yl)-6, 7,8, 9-tetrahydropyrido[1,2-a]indol-7-yl]-2-methoxy-ethanesulfonamide

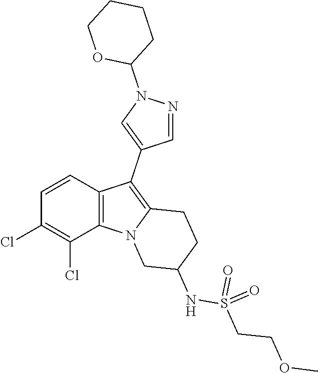 [SMILES: COCCS(=O)(=O)NC1CCc2c(-c3cnn(C4CCCCO4)c3)c3ccc(Cl)c(Cl)c3n2C1]

Dissolve 3,4-dichloro-10-(1-tetrahydropyran-2-ylpyrazol-4-yl)-6,7,8,9-tetrahydropyrido[1,2-a]indol-7-amine (100 mg, 0.234 mmol) and TEA (0.16 mL, 1.1 mmol) in DCM (2 mL) and add 2-methoxyethane-1-sulfonyl chloride (78 mg, 0.47 mmol). Stir at ambient temperature for 2 hrs. Dilute with water, extract with DCM, dry over anhydrous sodium sulfate, filter, and concentrate under vacuum. Purify by flash column chromatography eluting with MeOH in DCM to give N-[3,4-dichloro-10-(1-tetrahydropyran-2-ylpyrazol-4-yl)-6,7,8,9-tetrahydropyrido[1,2-a]indol-7-yl]-2-methoxy-ethanesulfonamide (85 mg, 62%) as a yellow solid. ES/MS (m/z): (³⁵Cl/³⁷Cl) 527.2/529.2 (M+H).

### EXAMPLE 117

### N-[3,4-Dichloro-10-(1H-pyrazol-4-yl)-6,7,8,9-tetrahydropyrido[1,2-a]indol-7-yl]-2-methoxy-ethanesulfonamide

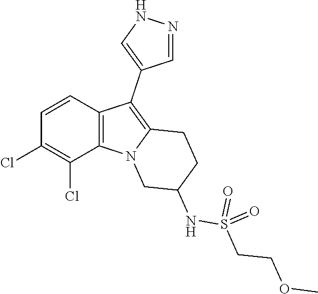 [SMILES: COCCS(=O)(=O)NC1CCc2c(-c3cn[nH]c3)c3ccc(Cl)c(Cl)c3n2C1]

Dissolve N-[3,4-dichloro-10-(1-tetrahydropyran-2-ylpyrazol-4-yl)-6,7,8,9-tetrahydropyrido[1,2-a]indol-7-yl]-2-methoxy-ethanesulfonamide (85 mg, 0.15 mmol) in DCM (2 mL) and add TFA (2 mL). Stir at ambient temperature for 2 hrs. Concentrate under vacuum and purify by prep-HPLC to give N-[3,4-dichloro-10-(1H-pyrazol-4-yl)-6,7,8,9-tetrahydropyrido[1,2-a]indol-7-yl]-2-methoxy-ethane sulfonamide (15.5 mg, 24%) as a white solid. ES/MS (m/z): (³⁵Cl/³⁷Cl) 443.3/445.2 (M+H).

### Intermediate 294

### Methyl 3-(N-(3,4-dichloro-10-(1-(tetrahydro-2H-pyran-2-yl)-1H-pyrazol-4-yl)-6,7,8,9-tetrahydropyrido[1,2-a]indol-7-yl)sulfamoyl)propanoate

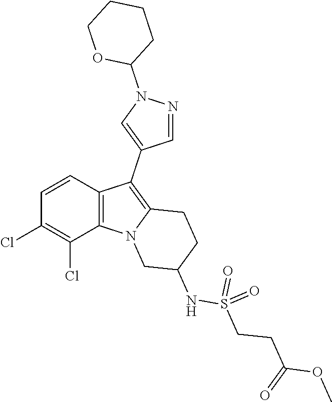 [SMILES: COC(=O)CCS(=O)(=O)NC1CCc2c(-c3cnn(C4CCCCO4)c3)c3ccc(Cl)c(Cl)c3n2C1]

Dissolve 3,4-dichloro-10-(1-(tetrahydro-2H-pyran-2-yl)-1H-pyrazol-4-yl)-6,7,8,9-tetrahydro pyrido[1,2-a]indol-7-amine (150 mg, 0.333 mmol) and TEA (0.25 mL, 1.8 mmol) in anhydrous DCM (10 mL). Add methyl 3-chlorosulfonylpropanoate (128 mg, 0.665 mmol) and stir at ambient temperature for 2 hrs. Combine with another batch run at 0.13×scale for work up and purification. Dilute with water, extract with DCM, wash with saturated aqueous NaCl, dry over anhydrous Na₂SO₄ and concentrate under vacuum. Purify by flash column chromatography eluting with EtOAc in petroleum ether to give methyl 3-(N-(3,4-dichloro-10-(1-(tetrahydro-2H-pyran-2-yl)-1H-pyrazol-4-yl)-6,7,8,9-tetrahydropyrido[1,2-a]indol-7-yl)sulfamoyl)propanoate (115 mg, 49%) as a yellow solid.

### Intermediate 295

### 3-(N-(3,4-Dichloro-10-(1-(tetrahydro-2H-pyran-2-yl)-1H-pyrazol-4-yl)-6,7,8,9-tetrahydropyrido[1,2-a]indol-7-yl)sulfamoyl)propanoic acid

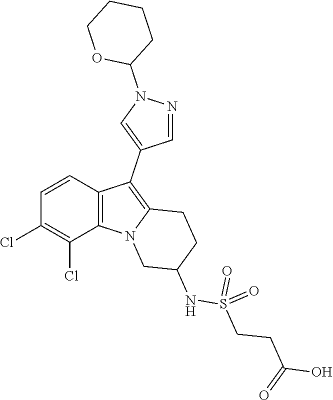 [SMILES: O=C(O)CCS(=O)(=O)NC1CCc2c(-c3cnn(C4CCCCO4)c3)c3ccc(Cl)c(Cl)c3n2C1]

Dissolve methyl 3-(N-(3,4-dichloro-10-(1-(tetrahydro-2H-pyran-2-yl)-1H-pyrazol-4-yl)-6,7,8,9-tetrahydropyrido[1,2-a]indol-7-yl)sulfamoyl)propanoate (115 mg, 0.186 mmol) in THF (2 mL). Add NaOH (38 mg, 0.92 mmol) in water (2 mL) and stir at ambient temperature for 2 hrs. Adjust to pH=4 with 1M aqueous HCl. Collect the solid by filtration and dry under vacuum to give 3-(N-(3,4-dichloro-10-(1-(tetrahydro-2H-pyran-2-yl)-1H-pyrazol-4-yl)-6,7,8,9-tetrahydropyrido[1,2-a]indol-7-yl)sulfamoyl)propanoic acid (100 mg, 89%) as a white solid.

### Intermediate 296

### 3-(N-(3,4-Dichloro-10-(1-(tetrahydro-2H-pyran-2-yl)-1H-pyrazol-4-yl)-6,7, 8,9-tetrahydropyrido[1,2-a]indol-7-yl)sulfamoyl)-N-methylpropanamide

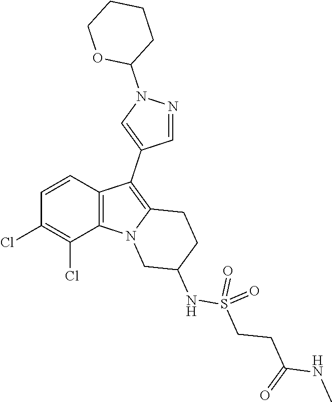 [SMILES: CNC(=O)CCS(=O)(=O)NC1CCc2c(-c3cnn(C4CCCCO4)c3)c3ccc(Cl)c(Cl)c3n2C1]

Dissolve 3-(N-(3,4-dichloro-10-(1-(tetrahydro-2H-pyran-2-yl)-1H-pyrazol-4-yl)-6,7,8,9-tetrahydropyrido[1,2-a]indol-7-yl)sulfamoyl)propanoic acid (100 mg, 0.166 mmol) and DIPEA (0.15 mL, 0.86 mmol) in DMF (3 mL). Add methylamine (0.2 mL, 0.4 mmol, 2M in THF) and HATU (130 mg, 0.335 mmol) and stir at ambient temperature for 16-18 hrs. Dilute with water and extract with EtOAc, wash with saturated aqueous NaCl, dry over anhydrous Na₂SO₄ and concentrate under vacuum. Purify by flash column chromatography eluting with MeOH in DCM to give 3-(N-(3,4-dichloro-10-(1-(tetrahydro-2H-pyran-2-yl)-1H-pyrazol-4-yl)-6,7,8,9-tetrahydropyrido[1,2-a]indol-7-yl)sulfamoyl)-N-methylpropanamide (70 mg, 70%) as a yellow gum. ES/MS (m/z): (³⁵Cl/³⁷Cl) 554.0/556.0 (M+H).

### EXAMPLE 118

### 3-(N-(3,4-Dichloro-10-(1H-pyrazol-4-yl)-6,7, 8,9-tetrahydropyrido[1,2-a]indol-7-yl)sulfamoyl)-N-methylpropanamide

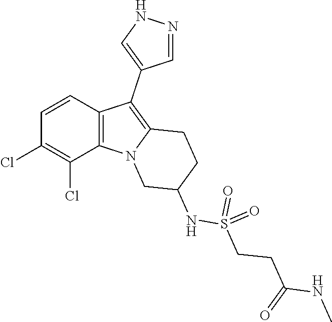 [SMILES: CNC(=O)CCS(=O)(=O)NC1CCc2c(-c3cn[nH]c3)c3ccc(Cl)c(Cl)c3n2C1]

Dissolve 3-(N-(3,4-dichloro-10-(1-(tetrahydro-2H-pyran-2-yl)-1H-pyrazol-4-yl)-6,7,8,9-tetrahydropyrido[1,2-a]indol-7-yl)sulfamoyl)-N-methylpropanamide (70 mg, 0.12 mmol) in DCM (2 mL). Add TFA (2 mL) and stir at ambient temperature for 2 hrs. Concentrate under vacuum and purify by prep-HPLC to give 3-(N-(3,4-dichloro-10-(1H-pyrazol-4-yl)-6,7,8,9-tetrahydropyrido[1,2-a]indol-7-yl)sulfamoyl)-N-methylpropanamide (23.2 mg, 43%) as a white solid. ES/MS (m/z): (³⁵Cl/³⁷Cl) 470.3/472.2 (M+H).

### Intermediate 297

### N-(3,4-Dichloro-10-(1-(tetrahydro-2H-pyran-2-yl)-1H-pyrazol-4-yl)-6,7,8,9-tetrahydropyrido[1,2-a]indol-7-yl)-2-(1,3-dioxoisindolin-2-yl)ethanesulfonamide

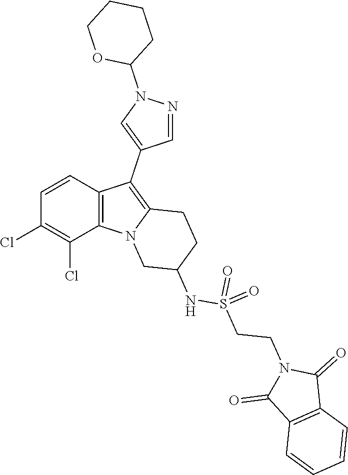 [SMILES: O=C1c2ccccc2C(=O)N1CCS(=O)(=O)NC1CCc2c(-c3cnn(C4CCCCO4)c3)c3ccc(Cl)c(Cl)c3n2C1]

Dissolve 3,4-dichloro-10-(1-(tetrahydro-2H-pyran-2-yl)-1H-pyrazol-4-yl)-6,7,8,9-tetrahydro pyrido[1,2-a]indol-7-amine (150 mg, 0.352 mmol) and TEA (0.25 mL, 1.8 mmol) in DCM (10 mL). Add 2-(1,3-dioxoisoindolin-2-yl)ethanesulfonyl chloride (193 mg, 0.703 mmol) and stir at ambient temperature for 2 hrs. Dilute with water, extract with DCM, wash with saturated aqueous NaCl, dry over anhydrous Na₂SO₄, and concentrate under vacuum. Purify by flash column chromatography eluting with EtOAc in petroleum ether to give methyl N-(3,4-dichloro-10-(1-(tetrahydro-2H-pyran-2-yl)-1H-pyrazol-4-yl)-6,7,8,9-tetrahydropyrido[1,2-a]indol-7-yl)-2-(1,3-dioxoisoindolin-2-yl)ethanesulfonamide (150 mg, 53%) as a yellow solid. ES/MS (m/z): (³⁵Cl/³⁷Cl) 641.9/643.9 (M+H).

### Intermediate 298

### 2-Amino-N-(3,4-dichloro-10-(1-(tetrahydro-2H-pyran-2-yl)-1H-pyrazol-4-yl)-6, 7,8,9-tetrahydropyrido[1,2-a]indol-7-yl)ethanesulfonamide

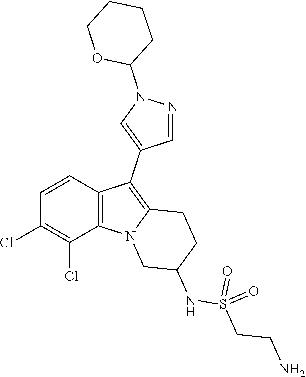 [SMILES: NCCS(=O)(=O)NC1CCc2c(-c3cnn(C4CCCCO4)c3)c3ccc(Cl)c(Cl)c3n2C1]

Dissolve N-(3,4-dichloro-10-(1-(tetrahydro-2H-pyran-2-yl)-1H-pyrazol-4-yl)-6,7,8,9-tetrahydropyrido[1,2-a]indol-7-yl)-2-(1,3-dioxoisoindolin-2-yl)ethanesulfonamide (150 mg, 0.187 mmol) in EtOH (10 mL). Add hydrazine hydrate (170 mg, 4.24 mmol, 80% purity) and stir at 85° C. for 16-18 hrs. Filter and wash the precipitate with EtOH. Concentrate the filtrate and dilute with water, extract with DCM, dry over anhydrous Na₂SO₄, filter, and concentrate under vacuum. Purify by flash column chromatography eluting with MeOH in DCM to give 2-amino-N-(3,4-dichloro-10-(1-(tetrahydro-2H-pyran-2-yl)-1H-pyrazol-4-yl)-6,7,8,9-tetrahydropyrido[1,2-a]indol-7-yl)ethanesulfonamide (84 mg, 81%) as colorless oil. ES/MS (m/z): (³⁵Cl/³⁷Cl) 512.0/514.1 (M+H).

### Intermediate 299

### N-(2-(N-(3,4-Dichloro-10-(1-(tetrahydro-2H-pyran-2-yl)-1H-pyrazol-4-yl)-6, 7,8,9-tetrahydropyrido[1,2-a]indol-7-yl)sulfamoyl)ethyl)acetamide

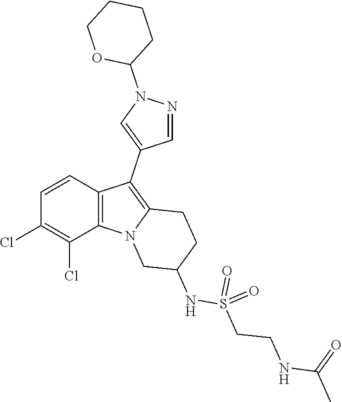 [SMILES: CC(=O)NCCS(=O)(=O)NC1CCc2c(-c3cnn(C4CCCCO4)c3)c3ccc(Cl)c(Cl)c3n2C1]

Dissolve give 2-amino-N-(3,4-dichloro-10-(1-(tetrahydro-2H-pyran-2-yl)-1H-pyrazol-4-yl)-6,7,8,9-tetrahydropyrido[1,2-a]indol-7-yl)ethanesulfonamide (50 mg, 0.078 mmol) and TEA (24 mg, 0.24 mmol) in DCM (5 mL). Add acetyl chloride (9 mg, 0.11 mmol) and stir at ambient temperature for 2 hrs. Dilute with water, extract with DCM, wash with saturated aqueous NaCl, dry over anhydrous Na₂SO₄, filter, and concentrate under vacuum to give N-(2-(N-(3,4-dichloro-10-(1-(tetrahydro-2H-pyran-2-yl)-1H-pyrazol-4-yl)-6,7,8,9-tetrahydropyrido[1,2-a]indol-7-yl)sulfamoyl)ethyl)acetamide (60 mg, 97%) as a yellow solid.

### EXAMPLE 119

### N-(2-(N-(3,4-Dichloro-10-(1H-pyrazol-4-yl)-6,7, 8,9-tetrahydropyrido[1,2-a]indol-7-yl)sulfamoyl)ethyl)acetamide

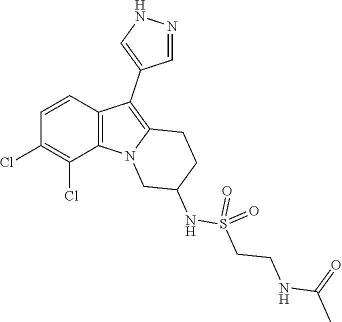 [SMILES: CC(=O)NCCS(=O)(=O)NC1CCc2c(-c3cn[nH]c3)c3ccc(Cl)c(Cl)c3n2C1]

Dissolve N-(2-(N-(3,4-dichloro-10-(1-(tetrahydro-2H-pyran-2-yl)-1H-pyrazol-4-yl)-6,7,8,9-tetrahydropyrido[1,2-a]indol-7-yl)sulfamoyl)ethyl)acetamide (60 mg, 0.076 mmol) in DCM (2 mL). Add TFA (2 mL) and stir at ambient temperature for 2 hrs. Concentrate under vacuum and purify by prep-HPLC to give N-(2-(N-(3,4-dichloro-10-(1H-pyrazol-4-yl)-6,7,8,9-tetrahydropyrido[1,2-a]indol-7-yl)sulfamoyl)ethyl)acetamide (25.48 mg, 71%) as a white solid. ES/MS (m/z): (³⁵Cl/³⁷Cl) 470.3/472.2 (M+H).

### EXAMPLE 120

### N-(3,4-Dichloro-10-(1H-pyrazol-4-yl)-6,7, 8,9-tetrahydropyrido[1,2-a]indol-7-yl)-2-(1H-tetrazol-1-yl)ethane-1-sulfonamide

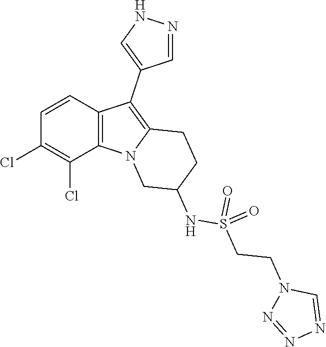 [SMILES: O=S(=O)(CCn1cnnn1)NC1CCc2c(-c3cn[nH]c3)c3ccc(Cl)c(Cl)c3n2C1]

Dissolve 2-amino-N-(3,4-dichloro-10-(1-(tetrahydro-2H-pyran-2-yl)-1H-pyrazol-4-yl)-6,7,8,9-tetrahydropyrido[1,2-a]indol-7-yl)ethane-1-sulfonamide (200 mg, 0.375 mmol) and trimethyl orthoformate (15 mL, 140 mmol) in acetic acid (10 mL) at 90° C. Add NaN₃ (170 mg, 2.61 mmol) and stir at 90° C. for 16-18 hrs. Dilute with water, extract with EtOAc, wash with saturated aqueous NaCl, dry over anhydrous Na₂SO₄, filter, and concentrate under vacuum. Purify by prep-HPLC to give N-(3,4-dichloro-10-(1H-pyrazol-4-yl)-6,7,8,9-tetrahydropyrido[1,2-a]indol-7-yl)-2-(1H-tetrazol-1-yl)ethane-1-sulfonamide (34.08 mg, 19%) as a pink solid. ES/MS (m/z): (³⁵Cl/³⁷Cl) 481.3/483.3 (M+H).

### Intermediate 300

### Ethyl (E)-4-((6,7-dichloro-3-(1H-pyrazol-4-yl)-1H-indol-4-yl)amino)but-2-enoate

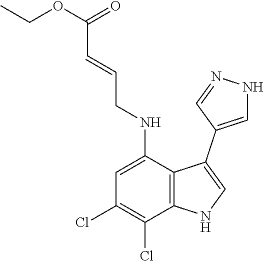 [SMILES: CCOC(=O)/C=C/CNc1cc(Cl)c(Cl)c2[nH]cc(-c3cn[nH]c3)c12]

Dissolve 6,7-dichloro-3-(1H-pyrazol-4-yl)-1H-indol-4-amine (200 mg, 0.621 mmol) in MeOH (10 mL). Add acetic acid (4 mg, 0.07 mmol) to adjust to pH=5 and stir at ambient temperature for 0.5 hr. Add ethyl trans-4-oxo-2-butenoate (241 mg, 1.86 mmol) and stir at ambient temperature for 0.5 hr. Add sodium cyanoborohydride (123 mg, 1.86 mmol) and stir at ambient temperature for 16-18 hrs. Concentrate and adjust to pH˜7 with saturated aqueous NaHCO₃. Extract with EtOAc, wash with saturated aqueous NaCl, dry over anhydrous Na₂SO₄, filter, and concentrate under vacuum. Purify by flash column chromatography eluting with EtOAc in petroleum ether to give ethyl (E)-4-((6,7-dichloro-3-(1H-pyrazol-4-yl)-1H-indol-4-yl)amino)but-2-enoate (170 mg, 61%) as a brown solid. ES/MS (m/z): (³⁵Cl/³⁷Cl) 379.1/381.1 (M+H).

### EXAMPLE 121

### (E)-4-((6,7-Dichloro-3-(1H-pyrazol-4-yl)-1H-indol-4-yl)amino)but-2-en-1-ol

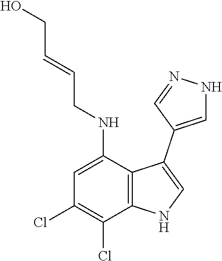 [SMILES: OC/C=C/CNc1cc(Cl)c(Cl)c2[nH]cc(-c3cn[nH]c3)c12]

Dissolve ethyl (E)-4-((6,7-dichloro-3-(1H-pyrazol-4-yl)-1H-indol-4-yl)amino)but-2-enoate (170 mg, 0.359 mmol) in DCM (8 mL). Add DIBAL-H (1.8 mL, 1.80 mmol, 1M in toluene) dropwise at −78° C. under N₂ atmosphere. Stir at −78° C. for 3 hrs. Quench with saturated aqueous potassium sodium tartrate. Combine with another batch run at 0.25×scale for work up and purification. Concentrate under vacuum and extract with DCM. Wash with saturated aqueous NaCl, dry over Na₂SO₄, filter, and concentrate under vacuum. Purify by prep-HPLC to give (E)-4-((6,7-dichloro-3-(1H-pyrazol-4-yl)-1H-indol-4-yl)amino)but-2-en-1-ol (30.5 mg, 20%) as a white solid. ES/MS (m/z): (³⁵Cl/³⁷Cl) 337.3/339.3 (M+H).

### Intermediate 301

### 6,7-Dichloro-N-(2,2-difluoroethyl)-3-(1-tetrahydropyran-2-ylpyrazol-4-yl)-1H-indol-4-amine

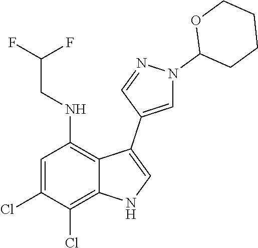 [SMILES: FC(F)CNc1cc(Cl)c(Cl)c2[nH]cc(-c3cnn(C4CCCCO4)c3)c12]

Suspend 4-bromo-6,7-dichloro-1-(p-tolylsulfonyl)-3-(1-tetrahydropyran-2-ylpyrazol-4-yl)indole (200 mg, 0.334 mmol), 2,2-difluoroethan-1-amine (45 mg, 0.50 mmol), t-BuONa (100 mg, 1.01 mmol) and (t-Bu₃P)₂Pd (26 mg, 0.050 mmol) in 1,4-dioxane (5 mL). Purge with N₂ then heat to 100° C. for 16-18 hrs. Concentrate under vacuum and purify by flash column chromatography eluting with EtOAc in petroleum ether to give 6,7-dichloro-N-(2,2-difluoroethyl)-3-(1-tetrahydropyran-2-ylpyrazol-4-yl)-1H-indol-4-amine (150 mg, 65%) as a yellow solid. ES/MS (m/z): (³⁵Cl/³⁷Cl) 415.2/417.1 (M+H).

### EXAMPLE 122

### 6, 7-Dichloro-N-(2,2-difluoroethyl)-3-(1H-pyrazol-4-yl)-1H-indol-4-amine

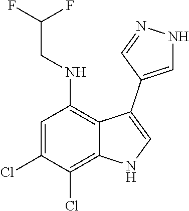 [SMILES: FC(F)CNc1cc(Cl)c(Cl)c2[nH]cc(-c3cn[nH]c3)c12]

Dissolve 6,7-dichloro-N-(2,2-difluoroethyl)-3-(1-tetrahydropyran-2-ylpyrazol-4-yl)-1H-indol-4-amine (150 mg, 0.217 mmol) in DCM (3 mL) and add TFA (3 mL) at 0° C. Stir at ambient temperature for 2 hrs. Combine with another batch run at 0.39×scale for work up and purification. Concentrate under vacuum and purify by prep-HPLC to give 6,7-dichloro-N-(2,2-difluoroethyl)-3-(1H-pyrazol-4-yl)-1H-indol-4-amine (28.70 mg, 29%) as an off-white solid. ES/MS (m/z): (³⁵Cl/³⁷Cl) 331.2/333.2 (M+H).

### Intermediate 302

### 6,7-Dichloro-N-(2-fluoroethyl)-1-(p-tolylsulfonyl)indol-4-amine

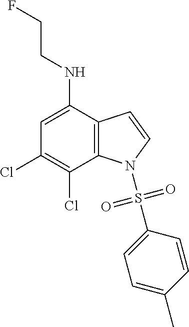 [SMILES: Cc1ccc(S(=O)(=O)n2ccc3c(NCCF)cc(Cl)c(Cl)c32)cc1]

Dissolve tert-butyl N-[6,7-dichloro-1-(p-tolylsulfonyl)indol-4-yl]carbamate (500 mg, 0.955 mmol) in DMF (10 mL) and add sodium hydride (85 mg, 2.1 mmol, 60% dispersion in mineral oil) at 0° C. Stir at 0° C. for 0.5 hr. Add 1-fluoro-2-iodoethane (228 mg, 1.25 mmol) at 0° C. and stir at ambient temperature for 16-18 hrs. Quench with water and extract with EtOAc (2×). Wash the combined organic layers with saturated aqueous NaCl, dry over anhydrous sodium sulfate, filter, and concentrate under vacuum. Purify by flash column chromatography eluting with EtOAc in petroleum ether to give 6,7-dichloro-N-(2-fluoroethyl)-1-(p-tolylsulfonyl)indol-4-amine (200 mg, 47%) as a yellow gum.

### Intermediate 303

### 6,7-Dichloro-N-(2-fluoroethyl)-1H-indol-4-amine

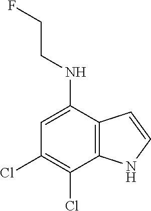 [SMILES: FCCNc1cc(Cl)c(Cl)c2[nH]ccc12]

Dissolve 6,7-dichloro-N-(2-fluoroethyl)-1-(p-tolylsulfonyl)indol-4-amine (200 mg, 0.448 mmol) in THF (5 mL) and add TBAF (1.50 mL, 1.50 mmol, 1M in THF). Stir at ambient temperature for 2 hrs. Dilute with EtOAc, wash with saturated aqueous NH₄Cl, dry over anhydrous sodium sulfate, filter, and concentrate under vacuum. Purify by flash column chromatography eluting with EtOAc in petroleum ether to give 6,7-dichloro-N-(2-fluoroethyl)-1H-indol-4-amine (135 mg, 99%) as a yellow solid.

### Intermediate 304

### 6,7-Dichloro-N-(2-fluoroethyl)-3-iodo-1H-indol-4-amine

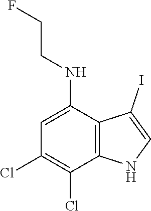 [SMILES: FCCNc1cc(Cl)c(Cl)c2[nH]cc(I)c12]

Dissolve 6,7-dichloro-N-(2-fluoroethyl)-1H-indol-4-amine (135 mg, 0.492 mmol) in DMF (3 mL) and add NIS (135 mg, 0.588 mmol) at 0° C. Stir at ambient temperature for 2 hrs. Quench with saturated aqueous Na₂SO₃ and extract with EtOAc (2×). Wash the combined organic layers with saturated aqueous NaCl, dry over anhydrous sodium sulfate, filter, and concentrate under vacuum. Purify by flash column chromatography eluting with EtOAc in petroleum ether to give 6,7-dichloro-N-(2-fluoroethyl)-3-iodo-1H-indol-4-amine (135 mg, 66%) as a yellow solid.

### Intermediate 305

### 6,7-Dichloro-N-(2-fluoroethyl)-3-(1-tetrahydropyran-2-ylpyrazol-4-yl)-1H-indol-4-amine

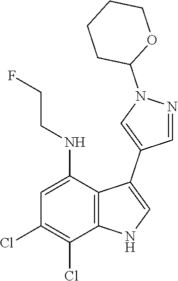 [SMILES: FCCNc1cc(Cl)c(Cl)c2[nH]cc(-c3cnn(C4CCCCO4)c3)c12]

Suspend 6,7-dichloro-N-(2-fluoroethyl)-3-iodo-1H-indol-4-amine (135 mg, 0.326 mmol) and 1-(tetrahydro-2H-pyran-2-yl)-4-(4,4,5,5-tetramethyl-1,3,2-dioxaborolan-2-yl)-1H-pyrazole (185 mg, 0.652 mmol), Pd(dtbpf)Cl₂ (43 mg, 0.065 mmol) and sodium carbonate (105 mg, 0.991 mmol) in 1,4-dioxane (4 mL) and water (1 mL). Purge with N₂ and heat to 90° C. for 2 hrs. Cool to ambient temperature and concentrate under vacuum. Purify by flash column chromatography eluting with EtOAc in petroleum ether to give 6,7-dichloro-N-(2-fluoroethyl)-3-(1-tetrahydropyran-2-ylpyrazol-4-yl)-1H-indol-4-amine (100 mg, 62%) as a yellow solid.

### EXAMPLE 123

### 6,7-Dichloro-N-(2-fluoroethyl)-3-(1H-pyrazol-4-yl)-1H-indol-4-amine

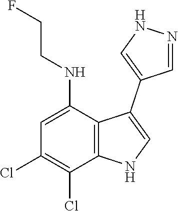 [SMILES: FCCNc1cc(Cl)c(Cl)c2[nH]cc(-c3cn[nH]c3)c12]

Dissolve 6,7-dichloro-N-(2-fluoroethyl)-3-(1-tetrahydropyran-2-ylpyrazol-4-yl)-1H-indol-4-amine (100 mg, 0.201 mmol) in DCM (2 mL) and add TFA (2 mL). Stir at ambient temperature for 2 hrs. Concentrate under vacuum and purify by prep-HPLC to give 6,7-dichloro-N-(2-fluoroethyl)-3-(1H-pyrazol-4-yl)-1H-indol-4-amine (24.18 mg, 38%) as a white solid. ES/MS (m/z): (³⁵Cl/³⁷Cl) 313.2/315.3 (M+H).

### Intermediate 306

### tert-Butyl N-[2-[2-[2-[3-[[3,4-dichloro-10-(1-tetrahydropyran-2-ylpyrazol-4-yl)-6,7,8,9-tetrahydropyrido[1,2-a]indol-7-yl]amino]-3-oxo-prop oxy]ethoxy]ethoxy]ethyl]carbamate

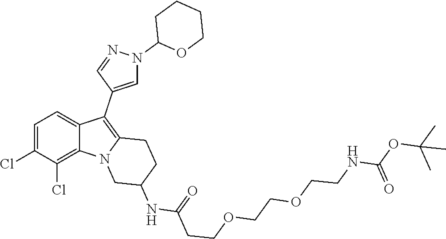 [SMILES: CC(C)(C)OC(=O)NCCOCCOCCC(=O)NC1CCc2c(-c3cnn(C4CCCCO4)c3)c3ccc(Cl)c(Cl)c3n2C1]

Suspend 3,4-dichloro-10-(1-tetrahydropyran-2-ylpyrazol-4-yl)-6,7,8,9-tetrahydropyrido[1,2-a]indol-7-amine (90 mg, 0.211 mmol), 2,2-dimethyl-4-oxo-3,8,11,14-tetraoxa-5-azaheptadecan-17-oic acid (143 mg, 0.423 mmol) and DIPEA (0.2 mL, 1 mmol) in DMF (2 mL). Add HATU (164 mg, 0.423 mmol). Stir at ambient temperature for 2 hrs. Dilute with water, extract with EtOAc, dry over anhydrous sodium sulfate, filter, and concentrate under vacuum. Purify by flash column chromatography eluting with MeOH in DCM to give tert-butyl N-[2-[2-[2-[3-[[3,4-dichloro-10-(1-tetrahydropyran-2-ylpyrazol-4-yl)-6,7,8,9-tetrahydropyrido[1,2-a]indol-7-yl]amino]-3-oxo-propoxy]ethoxy]ethoxy]ethyl]carbamate (110 mg, 64%) as a yellow solid. ES/MS (m/z): (³⁵Cl/³⁷Cl) 708.3/710.3 (M+H).

### EXAMPLE 124

### 3-[2-[2-(2-Aminoethoxy)ethoxy]ethoxy]-N-[3,4-dichloro-10-(1H-pyrazol-4-yl)-6, 7,8,9-tetrahydropyrido[1,2-a]indol-7-yl]propanamide

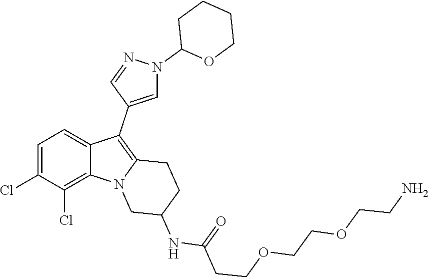 [SMILES: NCCOCCOCCC(=O)NC1CCc2c(-c3cnn(C4CCCCO4)c3)c3ccc(Cl)c(Cl)c3n2C1]

Dissolve tert-butyl N-[2-[2-[2-[3-[[3,4-dichloro-10-(1-tetrahydropyran-2-ylpyrazol-4-yl)-6,7,8,9-tetrahydropyrido[1,2-a]indol-7-yl]amino]-3-oxo-propoxy]ethoxy]ethoxy]ethyl]carbamate (110 mg, 0.135 mmol) in DCM (2 mL) and add TFA (2 mL). Stir at ambient temperature for 3 hrs. Concentrate under vacuum and dilute with water, adjust pH=12 with 2N aqueous NaOH, extract with EtOAc, dry over anhydrous sodium sulfate, filter, and concentrate under vacuum. Purify by reverse phase prep-HPLC to give 3-[2-[2-(2-aminoethoxy)ethoxy]ethoxy]-N-[3,4-dichloro-10-(1H-pyrazol-4-yl)-6,7,8,9-tetrahydropyrido[1,2-a]indol-7-yl]propanamide (20.63 mg, 27%) as a white solid. ES/MS (m/z): (³⁵Cl/³⁷Cl) 524.4/526.4 (M+H).

### Intermediate 307

### 4-(3-((tert-Butyldimethylsilyl)oxy)-2-methylpropoxy)-6,7-dichloro-1-tosyl-1H-indole

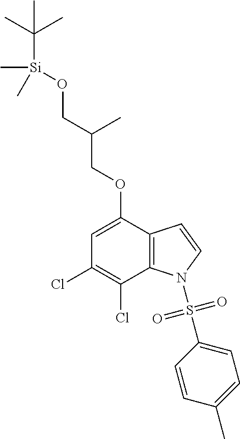 [SMILES: Cc1ccc(S(=O)(=O)n2ccc3c(OCC(C)CO[Si](C)(C)C(C)(C)C)cc(Cl)c(Cl)c32)cc1]

Dissolve 6,7-dichloro-1-(p-tolylsulfonyl)indol-4-ol (1.00 g, 2.67 mmol), 3-[tert-butyl(dimethyl) silyl]oxy-2-methyl-propan-1-ol (1.1 g, 5.1 mmol) and Ph₃P (1.78 g, 6.65 mmol) in anhydrous toluene (15 mL). Add DIAD (860 mg, 4.00 mmol) and stir at 110° C. for 16-18 hrs under N₂ atmosphere. Dilute with water and extract with EtOAc. Dry over anhydrous Na₂SO₄, filter, and concentrate under vacuum. Purify by flash column chromatography eluting with EtOAc in petroleum ether to give 4-(3-((tert-butyldimethylsilyl)oxy)-2-methylpropoxy)-6,7-dichloro-1-tosyl-1H-indole (1.30 g, 81%) as a colorless oil.

### Intermediate 308

### 3-(6,7-Dichloro-1H-indol-4-yl)oxy)-2-methylpropan-1-ol

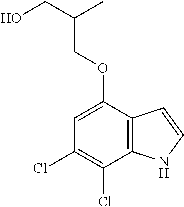 [SMILES: CC(CO)COc1cc(Cl)c(Cl)c2[nH]ccc12]

Dissolve 4-(3-((tert-butyldimethylsilyl)oxy)-2-methylpropoxy)-6,7-dichloro-1-tosyl-1H-indole (1.2 g, 2.0 mmol) in THF (2 mL). Add TBAF (10 mL, 10 mmol, 1M in THF) at 0° C. and stir at ambient temperature for 4 hrs under N₂ atmosphere. Dilute with EtOAc, wash with saturated aqueous NH₄Cl and saturated aqueous NaCl, dry over anhydrous Na₂SO₄, filter, and concentrate under vacuum. Purify by flash column chromatography eluting with EtOAc in petroleum ether to give 3-((6,7-dichloro-1H-indol-4-yl)oxy)-2-methylpropan-1-ol (359 mg, 59%) as a colorless oil.

### Intermediate 309

### 3-((6,7-Dichloro-3-iodo-1H-indol-4-yl)oxy)-2-methylpropan-1-ol

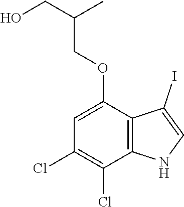 [SMILES: CC(CO)COc1cc(Cl)c(Cl)c2[nH]cc(I)c12]

Dissolve 3-((6,7-dichloro-1H-indol-4-yl)oxy)-2-methylpropan-1-ol (370 mg, 1.22 mmol) in DMF (3 mL). Add NIS (335 mg, 1.46 mmol) and stir at ambient temperature for 1.5 hrs. Concentrate under vacuum, dilute with EtOAc, wash with water, and extract the aqueous layer with EtOAc (3×). Wash the combined organic layers with saturated aqueous NaCl, dry over anhydrous Na₂SO₄, filter, and concentrate under vacuum. Purify by flash column chromatography eluting with EtOAc in petroleum ether to give 3-((6,7-dichloro-3-iodo-1H-indol-4-yl)oxy)-2-methylpropan-1-ol (415 mg, 77%) as a purple solid.

### Intermediate 310

### 3-((6,7-Dichloro-3-(1-(tetrahydro-2H-pyran-2-yl)-1H-pyrazol-4-yl)-1H-indol-4-yl)oxy)-2-methylpropan-1-ol

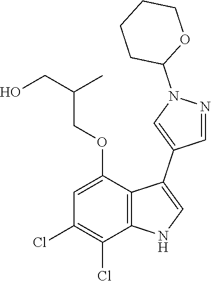 [SMILES: CC(CO)COc1cc(Cl)c(Cl)c2[nH]cc(-c3cnn(C4CCCCO4)c3)c12]

Suspend 3-((6,7-dichloro-3-iodo-1H-indol-4-yl)oxy)-2-methylpropan-1-ol (415 mg, 0.934 mmol), 1-tetrahydropyran-2-yl-4-(4,4,5,5-tetramethyl-1,3,2-dioxaborolan-2-yl)pyrazole (286 mg, 1.03 mmol), and Na₂CO₃ (297 mg, 2.80 mmol) in dioxane (4 mL) and water (1 mL). Add Pd(dtbpf)Cl₂ (124 mg, 0.186 mmol) at 25° C. under N₂ atmosphere. Purge with N₂ then stir at 90° C. for 1.5 hrs. Dilute with water and extract with EtOAc, wash with saturated aqueous NaCl, dry over anhydrous Na₂SO₄, filter, and concentrate under vacuum. Purify by flash column chromatography eluting with EtOAc in petroleum ether to give 3-((6,7-dichloro-3-(1-(tetrahydro-2H-pyran-2-yl)-1H-pyrazol-4-yl)-1H-indol-4-yl)oxy)-2-methylpropan-1-ol (120 mg, 24%) as a brown oil. ES/MS (m/z): (³⁵Cl/³⁷Cl) 424.0/426.0 (M+H).

### EXAMPLE 125

### 3-((6,7-Dichloro-3-(1H-pyrazol-4-yl)-1H-indol-4-yl)oxy)-2-methylpropan-1-ol

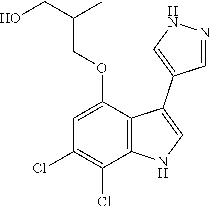 [SMILES: CC(CO)COc1cc(Cl)c(Cl)c2[nH]cc(-c3cn[nH]c3)c12]

Dissolve 3-((6, 7-dichloro-3-(1-(tetrahydro-2H-pyran-2-yl)-1H-pyrazol-4-yl)-1H-indol-4-yl)oxy)-2-methylpropan-1-ol (120 mg, 0.226 mmol) in THF (1 mL). Add 6M aqueous HCl (2 mL) and stir at ambient temperature for 1 hr. Concentrate under vacuum and purify by prep-HPLC to give 3-((6,7-dichloro-3-(1H-pyrazol-4-yl)-1H-indol-4-yl)oxy)-2-methylpropan-1-ol (34.35 mg, 44%) as a white solid. ES/MS (m/z): (³⁵Cl/³⁷Cl) 340.3/342.3 (M+H).

### Intermediate 311

### 6,7-Dichloro-4-((cis-3-fluorocyclobutoxy)-1-tosyl-1H-indole

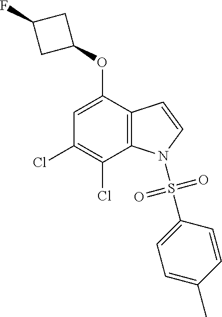 [SMILES: Cc1ccc(S(=O)(=O)n2ccc3c(O[C@H]4C[C@@H](F)C4)cc(Cl)c(Cl)c32)cc1]

Dissolve 6,7-dichloro-1-(p-tolylsulfonyl)indol-4-ol (1.00 g, 2.67 mmol), trans-3-fluorocyclobutanol (360 mg, 4.00 mmol) and Ph₃P (1.40 g, 5.30 mmol) in THF (20 mL). Add DIAD (1.1 g, 5.3 mmol) and stir at ambient temperature for 3 hrs. Dilute with water, extract with EtOAc, dry over anhydrous Na₂SO₄, filter, and concentrate under vacuum. Purify by flash column chromatography eluting with EtOAc in petroleum ether to give 6,7-dichloro-4-((cis-3-fluorocyclobutoxy)-1-tosyl-1H-indole (1.19 g, 91%) as a yellow oil. ES/MS (m/z): (³⁵Cl/³⁷Cl) 427.8/429.8 (M+H).

### Intermediate 312

### 6,7-Dichloro-4-(cis-3-fluorocyclobutoxy)-1H-indole

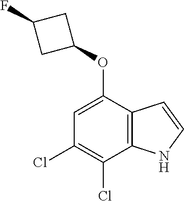 [SMILES: F[C@H]1C[C@@H](Oc2cc(Cl)c(Cl)c3[nH]ccc23)C1]

Dissolve 6,7-dichloro-4-((cis-3-fluorocyclobutoxy)-1-tosyl-1H-indole (1.09 g, 2.23 mmol) in THF (10 mL). Add TBAF (11 mL, 11 mmol, 1M in THF) and stir at ambient temperature for 3 hrs. Dilute with water, extract with EtOAc, wash with saturated aqueous NH₄Cl, dry over anhydrous Na₂SO₄, filter, and concentrate under vacuum. Purify by flash column chromatography eluting with EtOAc in petroleum ether to give 6,7-dichloro-4-(cis-3-fluorocyclobutoxy)-1H-indole (400 mg, 52%) as a yellow oil.

### Intermediate 313

### 6,7-Dichloro-4-(cis-3-fluorocyclobutoxy)-3-iodo-1H-indole

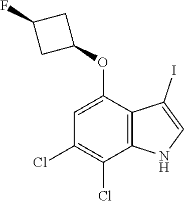 [SMILES: F[C@H]1C[C@@H](Oc2cc(Cl)c(Cl)c3[nH]cc(I)c23)C1]

Dissolve 6,7-dichloro-4-(cis-3-fluorocyclobutoxy)-1H-indole (400 mg, 1.17 mmol) in DMF (10 mL). Add NIS (295 mg, 1.29 mmol) and stir at ambient temperature for 1.5 hrs. Quench with saturated aqueous NaHCO₃, extract with EtOAc, wash with saturated aqueous NaCl, dry over anhydrous Na₂SO₄, filter, and concentrate under vacuum. Purify by flash column chromatography eluting with EtOAc in petroleum ether to give 6,7-dichloro-4-(cis-3-fluorocyclobutoxy)-3-iodo-1H-indole (150 mg, 26%) as a pink solid.

### Intermediate 314

### 6, 7-Dichloro-4-(cis-3-fluorocyclobutoxy)-3-(1-(tetrahydro-2H-pyran-2-yl)-1H-pyrazol-4-yl)-1H-indole

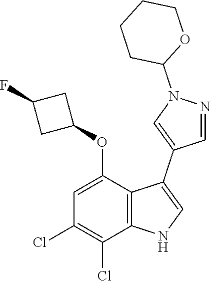 [SMILES: F[C@H]1C[C@@H](Oc2cc(Cl)c(Cl)c3[nH]cc(-c4cnn(C5CCCCO5)c4)c23)C1]

Suspend 6,7-dichloro-4-(cis-3-fluorocyclobutoxy)-3-iodo-1H-indole (150 mg, 0.300 mmol), 1-tetrahydropyran-2-yl-4-(4,4,5,5-tetramethyl-1,3,2-dioxaborolan-2-yl)pyrazole (176 mg, 0.601 mmol) and Na₂CO₃ (96 mg, 0.91 mmol) in dioxane (4 mL) and water (1 mL). Add Pd(dtbpf)Cl₂ (23 mg, 0.035 mmol) at 25° C. under N₂ atmosphere. Purge with N₂ and stir at 90° C. for 3 hrs. Dilute with water, extract with EtOAc, dry over anhydrous Na₂SO₄, filter, and concentrate under vacuum. Purify by flash column chromatography eluting with EtOAc in petroleum ether to give 6,7-dichloro-4-(cis-3-fluorocyclobutoxy)-3-(1-(tetrahydro-2H-pyran-2-yl)-1H-pyrazol-4-yl)-1H-indole (125 mg, 71%) as a brown solid. ES/MS (m/z): (³⁵Cl/³⁷Cl) 423.9/425.9 (M+H).

### EXAMPLE 126

### 6, 7-Dichloro-4-(cis-3-fluorocyclobutoxy)-3-(1H-pyrazol-4-yl)-1H-indole

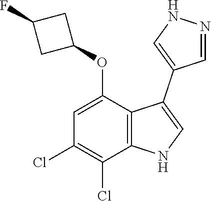 [SMILES: F[C@H]1C[C@@H](Oc2cc(Cl)c(Cl)c3[nH]cc(-c4cn[nH]c4)c23)C1]

Dissolve 6,7-dichloro-4-(cis-3-fluorocyclobutoxy)-3-(1-(tetrahydro-2H-pyran-2-yl)-1H-pyrazol-4-yl)-1H-indole (125 mg, 0.212 mmol) in DCM (3 mL). Add TFA (3 mL) and stir at ambient temperature for 2 hrs. Concentrate under vacuum and purify by prep-HPLC to give 6,7-dichloro-4-(cis-3-fluorocyclobutoxy)-3-(1H-pyrazol-4-yl)-1H-indole (17.12 mg, 24%) as a yellow solid. ES/MS (m/z): (³⁵Cl/³⁷Cl) 340.3/342.3 (M+H).

### Intermediate 315

### N′-Acetyl-6,7-dichloro-3-(1-(tetrahydro-2H-pyran-2-yl)-1H-pyrazol-4-yl)-1H-indole-2-carbohydrazide

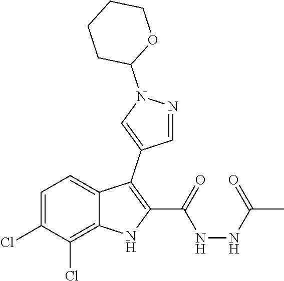 [SMILES: CC(=O)NNC(=O)c1[nH]c2c(Cl)c(Cl)ccc2c1-c1cnn(C2CCCCO2)c1]

Dissolve 6,7-dichloro-3-(1-tetrahydropyran-2-ylpyrazol-4-yl)-1H-indole-2-carboxylic acid (500 mg, 1.25 mmol), acetylhydrazide (293 mg, 3.76 mmol) and DIPEA (1.1 mL, 63 mmol) in DMF (10 mL). Add HATU (970 mg, 2.50 mmol) and stir at ambient temperature for 16-18 hrs. Dilute with water, extract with EtOAc, dry over anhydrous Na₂SO₄, filter, and concentrate under vacuum. Purify by flash column chromatography eluting with MeOH in DCM to give N′-acetyl-6,7-dichloro-3-(1-(tetrahydro-2H-pyran-2-yl)-1H-pyrazol-4-yl)-1H-indole-2-carbohydrazide (600 mg, 99%) as a yellow solid.

### EXAMPLE 127

### 2-(6,7-Dichloro-3-(1H-pyrazol-4-yl)-1H-indol-2-yl)-5-methyl-1,3,4-oxadiazole

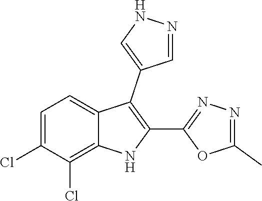 [SMILES: Cc1nnc(-c2[nH]c3c(Cl)c(Cl)ccc3c2-c2cn[nH]c2)o1]

Stir a solution of N′-acetyl-6,7-dichloro-3-(1-(tetrahydro-2H-pyran-2-yl)-1H-pyrazol-4-yl)-1H-indole-2-carbohydrazide (300 mg, 0.619 mmol) in POCl₃ (4 mL) at 65° C. for 2.5 hrs., under a N₂ atmosphere. Concentrate under vacuum and dissolve in DMSO. Purify by prep-HPLC to give 2-(6,7-dichloro-3-(1H-pyrazol-4-yl)-1H-indol-2-yl)-5-methyl-1,3,4-oxadiazole (41.74 mg, 20%) as a white solid. ES/MS (m/z): (³⁵Cl/³⁷Cl) 334.2/336.2 (M+H).

### Intermediate 316

### 6,7-Dichloro-3-(1-tetrahydropyran-2-ylpyrazol-4-yl)-N′-(2,2,2-trifluoroacetyl)-1H-indole-2-carbohydrazide

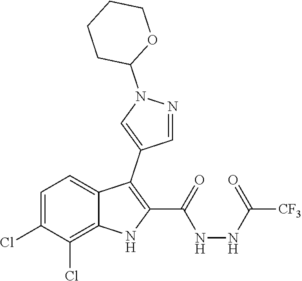 [SMILES: O=C(NNC(=O)C(F)(F)F)c1[nH]c2c(Cl)c(Cl)ccc2c1-c1cnn(C2CCCCO2)c1]

Dissolve 6,7-dichloro-3-(1-tetrahydropyran-2-ylpyrazol-4-yl)-1H-indole-2-carbohydrazide (800 mg, 1.95 mmol) and TEA (0.48 mL, 3.45 mmol) in DCM (15 mL). Add TFAA (0.38 mL, 2.71 mmol) at 0° C. under N₂ atmosphere. Stir at ambient temperature for 16-18 hrs. Quench with aqueous NaHCO₃ and extract with DCM. Wash with saturated aqueous NaCl, dry over anhydrous Na₂SO₄, filter, and concentrate under vacuum. Purify by flash column chromatography (EtOAc/PE) to give 6,7-dichloro-3-(1-tetrahydropyran-2-ylpyrazol-4-yl)-N′-(2,2,2-tri fluoroacetyl)-1H-indole-2-carbohydrazide (350 mg, 33%) as a brown solid. ES/MS (m/z): (³⁵Cl/³⁷Cl) 490.0/491.9 (M+H).

### Intermediate 317

### 2-(6,7-Dichloro-3-(1-(tetrahydro-2H-pyran-2-yl)-1H-pyrazol-4-yl)-1H-indol-2-yl)-5-(trifluoromethyl)-1,3,4-oxadiazole

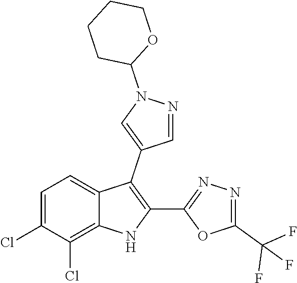 [SMILES: FC(F)(F)c1nnc(-c2[nH]c3c(Cl)c(Cl)ccc3c2-c2cnn(C3CCCCO3)c2)o1]

Dissolve 6,7-dichloro-3-(1-tetrahydropyran-2-ylpyrazol-4-yl)-N′-(2,2,2-trifluoroacetyl)-1H-indole-2-carbohydrazide (200 mg, 0.367 mmol) in acetonitrile (15 mL). Add p-toluenesulfonyl chloride (141 mg, 0.703 mmol) and DIPEA (136 mg, 1.05 mmol) at 0° C. under a N₂ atmosphere. Stir at ambient temperature for 16-18 hrs. Quench with water and extract with EtOAc. Wash with saturated aqueous NaCl, dry over anhydrous Na₂SO₄, filter, and concentrate under vacuum. Purify by flash column chromatography eluting with EtOAc in petroleum ether to give 2-(6,7-dichloro-3-(1-(tetrahydro-2H-pyran-2-yl)-1H-pyrazol-4-yl)-1H-indol-2-yl)-5-(trifluoromethyl)-1,3,4-oxadiazole (106 mg, 55%) as a yellow solid. ES/MS (m/z): (³⁵Cl/³⁷Cl) 472.0/473.8 (M+H).

### EXAMPLE 128

### 2-(6,7-Dichloro-3-(1H-pyrazol-4-yl)-1H-indol-2-yl)-5-(trifluoromethyl)-1,3,4-oxadiazole

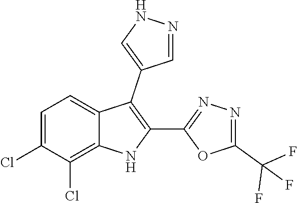 [SMILES: FC(F)(F)c1nnc(-c2[nH]c3c(Cl)c(Cl)ccc3c2-c2cn[nH]c2)o1]

Stir a solution of 2-[6,7-dichloro-3-(1-tetrahydropyran-2-ylpyrazol-4-yl)-1H-indol-2-yl]-5-(trifluoromethyl)-1,3,4-oxadiazole (131 mg, 0.247 mmol) in TFA (2 mL) and DCM (5 mL) at ambient temperature for 1 hr. Concentrate under vacuum and purify by prep-HPLC to give 2-(6,7-dichloro-3-(1H-pyrazol-4-yl)-1H-indol-2-yl)-5-(trifluoromethyl)-1,3,4-oxadiazole (27.68 mg, 28%) as a yellow solid. ES/MS (m/z): (³⁵Cl/³⁷Cl) 388.2/390.2 (M+H).

### Intermediate 318

### N-[[6, 7-Dichloro-3-(1-tetrahydropyran-2-ylpyrazol-4-yl)-1H-indol-2-yl]methyl] acetamide

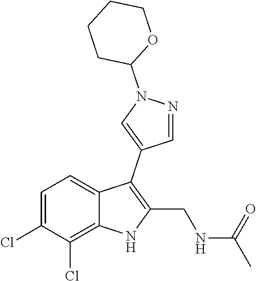 [SMILES: CC(=O)NCc1[nH]c2c(Cl)c(Cl)ccc2c1-c1cnn(C2CCCCO2)c1]

Dissolve [6,7-dichloro-3-(1-tetrahydropyran-2-ylpyrazol-4-yl)-1H-indol-2-yl]methanamine (150 mg, 0.377 mmol) and TEA (0.076 mL, 0.55 mmol) in DCM (5 mL) at 0° C. Add acetic anhydride (0.080 mL, 0.85 mmol). Stir at ambient temperature for 2 hrs. Dilute with water, extract with EtOAc (2×), wash the combined organic layers with saturated aqueous NaCl, dry over anhydrous Na₂SO₄, filter, and concentrate under vacuum. Purify by flash column chromatography eluting with EtOAc in petroleum ether to give N-[[6,7-dichloro-3-(1-tetrahydropyran-2-ylpyrazol-4-yl)-1H-indol-2-yl]methyl] acetamide (142 mg, 90%) as a white solid. ES/MS (m/z): (³⁵Cl/³⁷Cl) 407.1/409.0 (M+H).

### EXAMPLE 129

### N-[[6,7-Dichloro-3-(1H-pyrazol-4-yl)-1H-indol-2-yl]methyl]thioacetamide

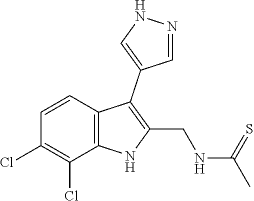 [SMILES: CC(=S)NCc1[nH]c2c(Cl)c(Cl)ccc2c1-c1cn[nH]c1]

Dissolve N-[[6,7-dichloro-3-(1-tetrahydropyran-2-ylpyrazol-4-yl)-1H-indol-2-yl] methyl] acetamide (142 mg, 0.338 mmol) in toluene (20 mL) and add Lawesson's reagent (218 mg, 0.523 mmol). Stir at 110° C. for 16 hrs. Dilute with EtOAc, wash with saturated aqueous NaCl, dry over with anhydrous Na₂SO₄, filter, and concentrate under vacuum. Purify by prep-HPLC to give N-[[6,7-dichloro-3-(1H-pyrazol-4-yl)-1H-indol-2-yl]methyl]thioacetamide (22.01 mg, 19%) as a white solid. ES/MS (m/z): 339.2 (M+H, small peak) and ES/MS (m/z): (³⁵Cl/³⁷Cl) 264.2/266.2 [M+H—CH₃C(S)NH₂].

### Intermediate 319

### (4-Bromo-6, 7-dichloro-1H-indol-2-yl)methanol

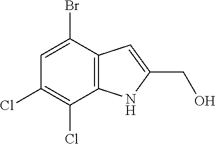 [SMILES: OCc1cc2c(Br)cc(Cl)c(Cl)c2[nH]1]

Dissolve ethyl 4-bromo-6,7-dichloro-1H-indole-2-carboxylate (2.5 g, 6.7 mmol) in THF (15 mL) and DCM (15 mL). Add DIBAL-H (30 mL, 30 mmol, 1.0M in toluene) at −78° C. under N₂ atmosphere. Stir at ambient temperature for 1 hr. Quench with saturated aqueous sodium potassium tartrate and stir at ambient temperature. Extract with EtOAc, wash with saturated aqueous NaCl, dry over anhydrous sodium sulfate, filter, and concentrate under vacuum. Purify by flash column chromatography eluting with EtOAc in petroleum ether to give (4-bromo-6,7-dichloro-1H-indol-2-yl)methanol (1.62 g, 78%) as a yellow solid. ES/MS (m/z): (292.0/294.0/296.0 (M−H).

### Intermediate 320

### 4-Bromo-6, 7-dichloro-2-(((tetrahydro-2H-pyran-2-yl)oxy)methyl)-1H-indole

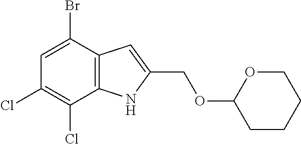 [SMILES: Clc1cc(Br)c2cc(COC3CCCCO3)[nH]c2c1Cl]

Dissolve (4-bromo-6,7-dichloro-1H-indol-2-yl)methanol (1.00 g, 3.05 mmol) and 3,4-dihydro-2H-pyran (0.56 mL, 6.1 mmol) in THF (8 mL). Add MeSO₃H (0.022 mL, 0.33 mmol) and stir at ambient temperature for 16-18 hrs. Quench with saturated aqueous sodium bicarbonate, extract with EtOAc, wash with saturated aqueous NaCl, dry over anhydrous sodium sulfate, filter, and concentrate under vacuum. Purify by flash column chromatography eluting with EtOAc in petroleum ether to give 4-bromo-6,7-dichloro-2-(((tetrahydro-2H-pyran-2-yl)oxy)methyl)-1H-indole (800 mg, 62%) as a yellow oil. ES/MS (m/z): 376.0/378.0/379.8 (M−H).

### Intermediate 321

### tert-Butyl (6,7-dichloro-2-(((tetrahydro-2H-pyran-2-yl)oxy)methyl)-1H-indol-4-yl)carbamate

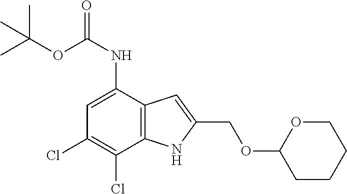 [SMILES: CC(C)(C)OC(=O)Nc1cc(Cl)c(Cl)c2[nH]c(COC3CCCCO3)cc12]

Dissolve 4-bromo-6,7-dichloro-2-(((tetrahydro-2H-pyran-2-yl)oxy)methyl)-1H-indole (1.54 g, 3.45 mmol), tert-butyl carbamate (660 mg, 5.52 mmol) and XantPhos-Pd-G₂ (340 mg, 0.342 mmol) in 1,4-dioxane (25 mL). Add Cs₂CO₃ (3.40 g, 10.4 mmol), purge with N₂ and stir at 100° C. for 16-18 hrs. Dilute with water, extract with EtOAc, wash with saturated aqueous NaCl, dry over anhydrous sodium sulfate, filter, and concentrate under vacuum. Purify by flash column chromatography eluting with EtOAc in petroleum ether to give tert-butyl (6,7-dichloro-2-(((tetrahydro-2H-pyran-2-yl)oxy)methyl)-1H-indol-4-yl)carbamate (1.13 g, 75%) as a yellow oil.

### Intermediate 322

### tert-Butyl (6,7-dichloro-3-iodo-2-(((tetrahydro-2H-pyran-2-yl)oxy)methyl)-1H-indol-4-yl)carbamate

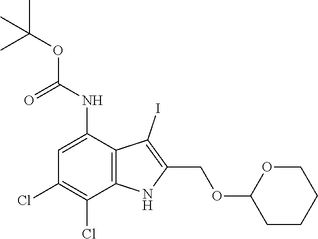 [SMILES: CC(C)(C)OC(=O)Nc1cc(Cl)c(Cl)c2[nH]c(COC3CCCCO3)c(I)c12]

Dissolve tert-butyl (6,7-dichloro-2-(((tetrahydro-2H-pyran-2-yl)oxy)methyl)-1H-indol-4-yl)carbamate (950 mg, 2.17 mmol) in DMF (15 mL). Add NIS (550 mg, 2.40 mmol) at 0° C. and stir at ambient temperature for 1 hr. Dilute with EtOAc, wash with water, saturated aqueous NaCl, dry over anhydrous sodium sulfate, filter, and concentrate under vacuum. Purify by flash column chromatography eluting with EtOAc in petroleum ether to give tert-butyl (6,7-dichloro-3-iodo-2-(((tetrahydro-2H-pyran-2-yl)oxy)methyl)-1H-indol-4-yl)carbamate (786 mg, 63%) as a yellow solid.

### Intermediate 323

### tert-Butyl (6,7-dichloro-3-(1-(tetrahydro-2H-pyran-2-yl)-1H-pyrazol-4-yl)-2-(((tetrahydro-2H-pyran-2-yl)oxy)methyl)-1H-indol-4-yl)carbamate

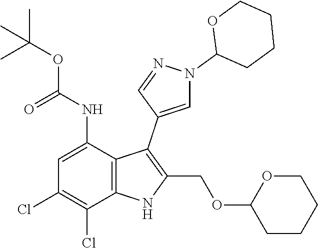 [SMILES: CC(C)(C)OC(=O)Nc1cc(Cl)c(Cl)c2[nH]c(COC3CCCCO3)c(-c3cnn(C4CCCCO4)c3)c12]

Dissolve tert-butyl (6,7-dichloro-3-iodo-2-(((tetrahydro-2H-pyran-2-yl)oxy)methyl)-1H-indol-4-yl)carbamate (786 mg, 1.38 mmol), 1-tetrahydropyran-2-yl-4-(4,4,5,5-tetramethyl-1,3,2-dioxaborolan-2-yl)pyrazole (576 mg, 2.07 mmol) and Pd(dtbpf)Cl₂ (184 mg, 0.277 mmol) in 1,4-dioxane (16 mL) and water (4 mL). Add Na₂CO₃ (514 mg, 4.83 mmol), purge with N₂ and stir at 90° C. for 5 hrs. Dilute with water, extract with EtOAc, dry over anhydrous sodium sulfate, filter, and concentrate under vacuum. Purify by flash column chromatography eluting with EtOAc in petroleum ether to give tert-butyl (6,7-dichloro-3-(1-(tetrahydro-2H-pyran-2-yl)-1H-pyrazol-4-yl)-2-(((tetrahydro-2H-pyran-2-yl)oxy)methyl)-1H-indol-4-yl)carbamate (590 mg, 72%) as a yellow solid.

### Intermediate 324

### (4-Amino-6,7-dichloro-3-(1H-pyrazol-4-yl)-1H-indol-2-yl)methanol

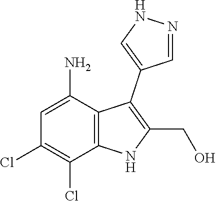 [SMILES: Nc1cc(Cl)c(Cl)c2[nH]c(CO)c(-c3cn[nH]c3)c12]

Dissolve tert-butyl (6,7-dichloro-3-(1-(tetrahydro-2H-pyran-2-yl)-1H-pyrazol-4-yl)-2-(((tetrahydro-2H-pyran-2-yl)oxy)methyl)-1H-indol-4-yl)carbamate (300 mg, 0.504 mmol) in THF (5 mL). Add 6M aqueous HCl (5 mL) and stir at ambient temperature for 4 hrs. Filter and dry the filter cake under vacuum to give (4-amino-6,7-dichloro-3-(1H-pyrazol-4-yl)-1H-indol-2-yl)methanol (110 mg, 70%) as a white solid.

### EXAMPLE 130

### N-(6,7-Dichloro-2-(hydroxymethyl)-3-(1H-pyrazol-4-yl)-1H-indol-4-yl)acetamide

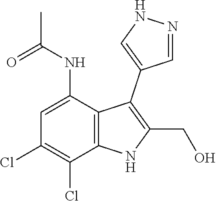 [SMILES: CC(=O)Nc1cc(Cl)c(Cl)c2[nH]c(CO)c(-c3cn[nH]c3)c12]

Dissolve (4-amino-6,7-dichloro-3-(1H-pyrazol-4-yl)-1H-indol-2-yl)methanol (51 mg, 0.16 mmol), DIPEA (0.71 mL, 4.10 mmol) and DMAP (20 mg, 0.16 mmol) in DCM (3 mL). Add acetic anhydride (0.28 mL, 3.0 mmol) at 0° C. and stir at ambient temperature for 16-18 hrs. Quench with saturated aqueous sodium bicarbonate, extract with DCM, wash with saturated aqueous NaCl, dry over anhydrous sodium sulfate, filter, and concentrate under vacuum. Dissolve the residue in MeOH (3 mL) and add potassium carbonate (225 mg, 1.63 mmol) at 0° C., stir at ambient temperature for 16-18 hrs. Dilute with water and extract with EtOAc, dry over anhydrous sodium sulfate, filter, and concentrate under vacuum. Purify by prep-HPLC to give N-(6,7-dichloro-2-(hydroxymethyl)-3-(1H-pyrazol-4-yl)-1H-indol-4-yl)acetamide (13.8 mg, 25%) as a white solid. ES/MS (m/z): (³⁵Cl/³⁷Cl) 339.3/341.2 (M+H).

### EXAMPLE 131

### N-(6,7-Dichloro-2-(hydroxymethyl)-3-(1H-pyrazol-4-yl)-1H-indol-4-yl)-2,2-difluoroacetamide

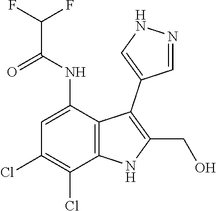 [SMILES: O=C(Nc1cc(Cl)c(Cl)c2[nH]c(CO)c(-c3cn[nH]c3)c12)C(F)F]

Dissolve [4-amino-6,7-dichloro-3-(1H-pyrazol-4-yl)-1H-indol-2-yl]methanol (150 mg, 0.429 mmol), DIPEA (0.84 mL, 4.80 mmol) and DMAP (12 mg, 0.098 mmol) in DCM (6 mL). Add difluoroacetic anhydride (602 mg, 3.36 mmol) at 0° C. dropwise and stir at ambient temperature 3 hrs. Quench with saturated aqueous sodium bicarbonate, extract with DCM and EtOAc, dry combined organic layers over anhydrous sodium sulfate, filter, and concentrate under vacuum. Purify by prep-HPLC to give N-(6,7-dichloro-2-(hydroxymethyl)-3-(1H-pyrazol-4-yl)-1H-indol-4-yl)-2,2-difluoroacetamide (60.04 mg, 37%) as a white solid. ES/MS (m/z): (³⁵Cl/³⁷Cl) 375.2/377.2 (M+H).

### EXAMPLE 132

### N-(6,7-Dichloro-2-(methoxymethyl)-3-(1H-pyrazol-4-yl)-1H-indol-4-yl)-2,2-difluoroacetamide

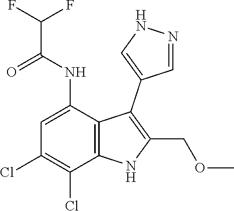 [SMILES: COCc1[nH]c2c(Cl)c(Cl)cc(NC(=O)C(F)F)c2c1-c1cn[nH]c1]

Dissolve [4-amino-6,7-dichloro-3-(1H-pyrazol-4-yl)-1H-indol-2-yl]methanol (70 mg, 0.22 mmol), DIPEA (0.31 mL, 1.8 mmol) and DMAP (5.4 mg, 0.044 mmol) in DCM (3.5 mL). Add difluoroacetic anhydride (321 mg, 1.79 mmol) at 0° C. and stir at ambient temperature for 2 hrs. Quench with saturated aqueous sodium bicarbonate, extract with DCM, dry over anhydrous sodium sulfate, filter, and concentrate under vacuum. Dissolve the residue in MeOH (3.5 mL), add potassium carbonate (309 mg, 2.24 mmol) and stir at ambient temperature for 16-18 hrs. Dilute with water, extract with EtOAc, dry over anhydrous sodium sulfate, filter, and concentrate under vacuum. Purify by prep-HPLC to give N-(6,7-dichloro-2-(methoxymethyl)-3-(1H-pyrazol-4-yl)-1H-indol-4-yl)-2,2-difluoroacetamide (5.06 mg, 6%) as a yellow solid. ES/MS (m/z): (³⁵Cl/³⁷Cl) 389.2/391.2 (M+H).

### Intermediate 325

### tert-Butyl N-[[6,7-dichloro-3-(2-tetrahydropyran-2-yl-2H-imidazol-4-yl)-1H-indol-2-yl]methyl]carbamate

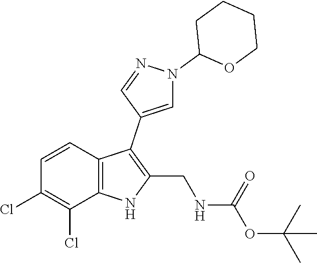 [SMILES: CC(C)(C)OC(=O)NCc1[nH]c2c(Cl)c(Cl)ccc2c1-c1cnn(C2CCCCO2)c1]

Dissolve [6,7-dichloro-3-(1-tetrahydropyran-2-ylpyrazol-4-yl)-1H-indol-2-yl]methanamine (1.20 g, 2.80 mmol) and TEA (1.2 mL, 8.60 mmol) in DCM (50 mL) at 0° C. Add Boc₂O (1.2 mL, 5.60 mmol). Stir at ambient temperature for 3 hrs. Combine with another batch run at 0.17×scale for work up and purification. Quench with water, extract with DCM, wash with saturated aqueous NaCl, dry over anhydrous Na₂SO₄, filter, and concentrate under vacuum. Purify by flash column chromatography eluting with EtOAc in petroleum ether to give tert-butyl N-[[6,7-dichloro-3-(2-tetrahydropyran-2-yl-2H-imidazol-4-yl)-1H-indol-2-yl]methyl]carbamate (1.02 g, 61%) as a yellow solid. ES/MS (m/z): (³⁵Cl/³⁷Cl) 465.1/466.8 (M+H).

### Intermediate 326

### tert-Butyl N-[[6, 7-dichloro-1-methyl-3-(2-tetrahydropyran-2-yl-2H-imidazol-4-yl) indol-2-yl]methyl]carbamate

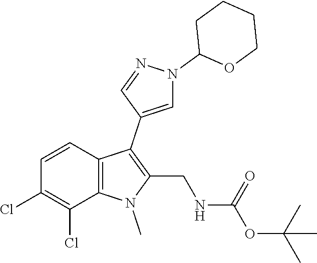 [SMILES: Cn1c(CNC(=O)OC(C)(C)C)c(-c2cnn(C3CCCCO3)c2)c2ccc(Cl)c(Cl)c21]

Suspend tert-butyl N-[[6,7-dichloro-3-(2-tetrahydropyran-2-yl-2H-imidazol-4-yl)-1H-indol-2-yl]methyl]carbamate (1.00 g, 1.95 mmol) and Cs₂CO₃ (2.23 g, 6.84 mmol) in DMF (24 mL) at 0° C. Add MeI (0.336 g, 2.34 mmol) and stir at ambient temperature for 3 hrs. Dilute with EtOAc, wash with water and saturated aqueous NaCl, dry over with anhydrous Na₂SO₄, filter, and concentrate under vacuum. Purify by flash column chromatography eluting with EtOAc in petroleum ether to give tert-butyl N-[[6,7-dichloro-1-methyl-3-(2-tetrahydropyran-2-yl-2H-imidazol-4-yl) indol-2-yl] methyl]carbamate (850 mg, 83%) as a yellow solid. ES/MS (m/z): (³⁵Cl/³⁷Cl) 479.0/480.9 (M+H).

### Intermediate 327

### [6,7-Dichloro-1-methyl-3-(1H-pyrazol-4-yl)indol-2-yl]methanamine hydrochloride

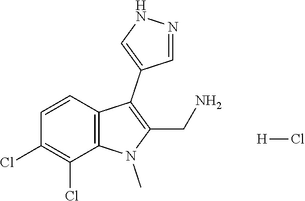 [SMILES: Cn1c(CN)c(-c2cn[nH]c2)c2ccc(Cl)c(Cl)c21 ; Cl]

Dissolve tert-butyl N-[[6,7-dichloro-1-methyl-3-(2-tetrahydropyran-2-yl-2H-imidazol-4-yl) indol-2-yl]methyl]carbamate (850 mg, 1.63 mmol) in 4M HCl in MeOH (3 mL). Stir at ambient temperature for 1 hr. Filter and dry under vacuum to give [6,7-dichloro-1-methyl-3-(1H-pyrazol-4-yl)indol-2-yl] methanamine (600 mg, 88%, HCl salt) as an orange solid. ES/MS (m/z): (³⁵Cl/³⁷Cl) 278.1/280.0 (M+H—NH₃).

### EXAMPLE 133

### N-[[6,7-Dichloro-1-methyl-3-(1H-pyrazol-4-yl)indol-2-yl]methyl]-2-hydroxy-acetamide

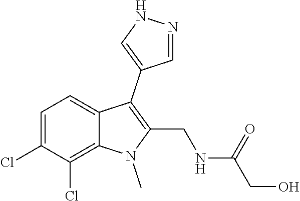 [SMILES: Cn1c(CNC(=O)CO)c(-c2cn[nH]c2)c2ccc(Cl)c(Cl)c21]

Suspend [6,7-dichloro-1-methyl-3-(1H-pyrazol-4-yl)indol-2-yl]methanamine (200 mg, 0.476 mmol, HCl salt) and TEA (0.332 mL, 2.38 mmol) in DCM (4 mL) at 0° C. Add acetoxyacetyl chloride (0.154 mL, 1.43 mmol). Stir at ambient temperature for 2 hrs. Filter and concentrate under vacuum. Dissolve the residue in MeOH (2 mL) and add K₂CO₃ (6 60 mg, 4.77 mmol). Stir at ambient temperature for 1 hr. Dilute with EtOAc, wash with saturated aqueous NaCl, dry over anhydrous Na₂SO₄, filter, and concentrate under vacuum. Triturate with DCM/MeOH (4 mL, 3/1) to give N-[[6,7-dichloro-1-methyl-3-(1H-pyrazol-4-yl)indol-2-yl]methyl]-2-hydroxy-acetamide (140 mg, 80%) as a white solid. ES/MS (m/z): (³⁵Cl/³⁷Cl) 352.9/354.9 (M+H).

### EXAMPLE 134

### N-[[6,7-Dichloro-1-methyl-3-(1H-pyrazol-4-yl) indol-2-yl]methyl]acetamide

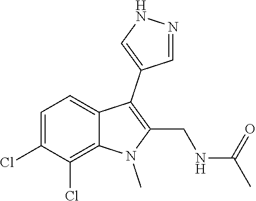 [SMILES: CC(=O)NCc1c(-c2cn[nH]c2)c2ccc(Cl)c(Cl)c2n1C]

Dissolve [6,7-dichloro-1-methyl-3-(1H-pyrazol-4-yl)indol-2-yl]methanamine (200 mg, 0.476 mmol, HCl salt) and TEA (0.241 mL, 1.73 mmol) in DCM (4 mL) at 0° C. Add acetic anhydride (0.135 mL, 1.43 mmol). Stir at ambient temperature for 2 hrs. Filter and concentrate under vacuum. Dissolve the residue in MeOH (2 mL). Add K₂CO₃ (₆60 mg, 4.78 mmol) and stir at ambient temperature for 1 hr. Dilute with EtOAc, wash with saturated aqueous NaCl, dry over saturated Na₂SO₄, filter, and concentrate under vacuum. Purify by flash column chromatography eluting with MeOH/DCM. Triturate with CH₃CN (3 mL) to give N-[[6,7-dichloro-1-methyl-3-(1H-pyrazol-4-yl) indol-2-yl]methyl]acetamide (68.2 mg, 41%) as a white solid. ES/MS (m/z): (³⁵Cl/³⁷Cl) 337.0/338.9 (M+H).

### Intermediate 328

### 9-Bromo-6,7-dichloro-2-(2-trimethylsilylethoxymethyl)-3,4-dihydropyrazino[1,2-a]indol-1-one

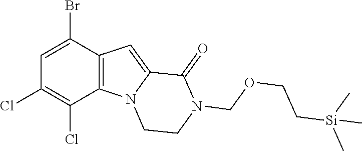 [SMILES: C[Si](C)(C)CCOCN1CCn2c(cc3c(Br)cc(Cl)c(Cl)c32)C1=O]

Dissolve 9-bromo-6,7-dichloro-3,4-dihydro-2H-pyrazino[1,2-a]indol-1-one (1.17 g, 3.33 mmol) in DMF (10 mL). Add sodium hydride (270 mg, 6.75 mmol, 60% dispersion in mineral oil) at 0° C. and stir at 0° C. for 0.5 hr. Add SEM-Cl (1.2 mL, 6.8 mmol) dropwise at 0° C. and stir at ambient temperature for 2 hrs. Quench with water, extract with EtOAc, wash with 4% aqueous LiCl, dry over anhydrous sodium sulfate, filter, and concentrate under vacuum. Purify by flash column chromatography eluting with EtOAc in petroleum ether to give 9-bromo-6,7-dichloro-2-(2-trimethylsilylethoxymethyl)-3,4-dihydropyrazino[1,2-a]indol-1-one (570 mg, 26%) as a colorless oil.

### Intermediate 329

### tert-Butyl N-((6,7-dichloro-1-oxo-2-(2-trimethylsilylethoxymethyl)-3,4-dihydropyrazino[1,2-a]indol-9-yl]carbamate

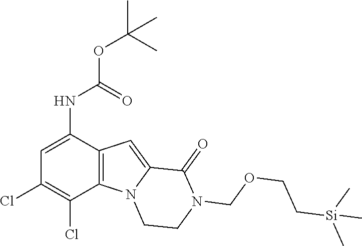 [SMILES: CC(C)(C)OC(=O)Nc1cc(Cl)c(Cl)c2c1cc1n2CCN(COCC[Si](C)(C)C)C1=O]

Dissolve 9-bromo-6,7-dichloro-2-(2-trimethylsilylethoxymethyl)-3,4-dihydropyrazino[1,2-a]indol-1-one (570 mg, 0.859 mmol) and tert-butyl carbamate (230 mg, 1.96 mmol) in 1,4-dioxane (10 mL). Add Pd₂(dba)₃ (62 mg, 0.067 mmol), XantPhos (77 mg, 0.13 mmol) and Cs₂CO₃ (550 mg, 1.69 mmol). Purge with N₂ and stir for 16-18 hrs at 100° C. under N₂ atmosphere. Quench with water, extract with EtOAc, dry over anhydrous sodium sulfate, filter, and concentrate under vacuum. Purify by flash column chromatography eluting with EtOAc in petroleum ether to give tert-butyl N-[6,7-dichloro-1-oxo-2-(2-trimethylsilylethoxymethyl)-3,4-dihydropyrazino[1,2-a]indol-9-yl]carbamate (440 mg, 92%) as a yellow gum.

### Intermediate 330

### tert-Butyl N-(cyanomethyl)-N-[6,7-dichloro-1-oxo-2-(2-trimethylsilylethoxymethyl)-3,4-dihydropyrazino[1,2-a]indol-9-yl]carbamate

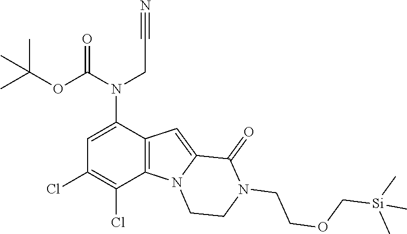 [SMILES: CC(C)(C)OC(=O)N(CC#N)c1cc(Cl)c(Cl)c2c1cc1n2CCN(CCOC[Si](C)(C)C)C1=O]

Dissolve tert-butyl N-[6,7-dichloro-1-oxo-2-(2-trimethylsilylethoxymethyl)-3,4-dihydropyrazino[1,2-a]indol-9-yl]carbamate (440 mg, 0.791 mmol) in DMF (5 mL). Add sodium hydride (55 mg, 1.4 mmol, 60% dispersion in mineral oil) and stir for 0.5 hr. Add bromoacetonitrile (215 mg, 1.79 mmol) dropwise. Stir at ambient temperature for 1 hr. Quench with water and extract the aqueous layer with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give tert-butyl N-(cyanomethyl)-N-[6,7-dichloro-1-oxo-2-(2-trimethylsilylethoxymethyl)-3,4-dihydropyrazino[1,2-a]indol-9-yl]carbamate (200 mg, 45%) as a yellow oil.

### Intermediate 331

### tert-Butyl N-[10-bromo-6, 7-dichloro-1-oxo-2-(2-trimethyl silyl ethoxymethyl)-3,4-dihydropyrazino[1,2-a]indol -9-yl]-N-(cyanomethyl)carbamate

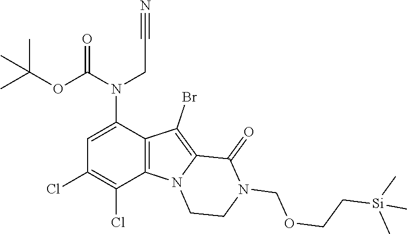 [SMILES: CC(C)(C)OC(=O)N(CC#N)c1cc(Cl)c(Cl)c2c1c(Br)c1n2CCN(COCC[Si](C)(C)C)C1=O]

Dissolve tert-butyl N-(cyanomethyl)-N-[6,7-dichloro-1-oxo-2-(2-trimethylsilylethoxymethyl)-3,4-dihydropyrazino[1,2-a]indol-9-yl]carbamate (200 mg, 0.352 mmol) in DMF (5 mL). Add NBS (100 mg, 0.562 mmol) and stir at ambient temperature for 2 hrs. Quench with saturated aqueous Na₂SO₃ and extract the aqueous layer with EtOAc (3×). Wash the combined organic layers with 4% aqueous LiCl, dry over anhydrous sodium sulfate, filter, and concentrate under vacuum to give tert-butyl N-[10-bromo-6,7-dichloro-1-oxo-2-(2-trimethylsilylethoxymethyl)-3,4-dihydropyrazino[1,2-a]indol-9-yl]-N-(cyanomethyl)carbamate (197 mg, 86%) as a brown gum. ES/MS (m/z): 617.0/619.0/621.0 (M+H).

### Intermediate 332

### tert-Butyl N-(cyanomethyl)-N-[6,7-dichloro-1-oxo-10-(1-tetrahydropyran-2-ylpyrazol-4-yl)-2-(2-trimethylsilylethoxymethyl)-3,4-dihydropyrazino[1,2-a]indol-9-yl]carbamate

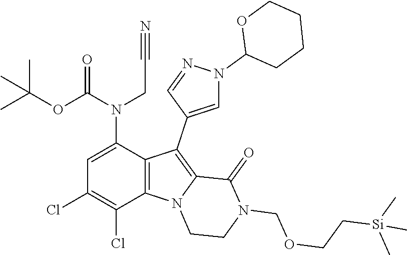 [SMILES: CC(C)(C)OC(=O)N(CC#N)c1cc(Cl)c(Cl)c2c1c(-c1cnn(C3CCCCO3)c1)c1n2CCN(COCC[Si](C)(C)C)C1=O]

Suspend tert-butyl N-[10-bromo-6,7-dichloro-1-oxo-2-(2-trimethylsilylethoxymethyl)-3,4-dihydropyrazino[1,2-a]indol-9-yl]-N-(cyanomethyl)carbamate (197 mg, 0.303 mmol), 1-(tetrahydro-2H-pyran-2-yl)-4-(4,4,5,5-tetramethyl-1,3,2-dioxaborolan-2-yl)-1H-pyrazole (180 mg, 0.615 mmol), Na₂CO₃ (70 mg, 0.660 mmol) and Pd(dppf)Cl₂ (25 mg, 0.032 mmol) in 1,4-dioxane (4 mL) and water (1 mL). Purge with N₂ and heat to 90° C. for 4 hrs. Cool to ambient temperature, dilute with water, and extract the aqueous layer with EtOAc (3×). Dry the combined organic layers over anhydrous sodium sulfate, filter, and concentrate under vacuum. Purify by flash column chromatography eluting with EtOAc in petroleum ether to give tert-butyl N-(cyanomethyl)-N-[6,7-dichloro-1-oxo-10-(1-tetrahydropyran-2-ylpyrazol-4-yl)-2-(2-trimethylsilylethoxymethyl)-3,4-dihydropyrazino[1,2-a]indol-9-yl]carbamate (80 mg, 34%) as a yellow gum. ES/MS (m/z): (³⁵Cl/³⁷Cl) 689.2/691.2 (M+H).

### EXAMPLE 135

### 2-[[6,7-Dichloro-1-oxo-10-(1H-pyrazol-4-yl)-3,4-dihydro-2H-pyrazino[1,2-a]indol-9-yl]amino]acetonitrile

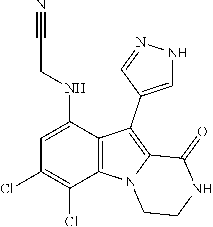 [SMILES: N#CCNc1cc(Cl)c(Cl)c2c1c(-c1cn[nH]c1)c1n2CCNC1=O]

Suspend tert-butyl N-(cyanomethyl)-N-[6,7-dichloro-1-oxo-10-(1-tetrahydropyran-2-ylpyrazol-4-yl)-2-(2-trimethylsilylethoxymethyl)-3,4-dihydropyrazino[1,2-a]indol-9-yl]carbamate (35 mg, 0.046 mmol) in DCM (1 mL). Add TFA (0.5 mL) and stir at ambient temperature for 2 hrs. Concentrate under vacuum and dissolve the residue in THF (1 mL) and water (0.5 mL), then add LiOH (300 mg, 12.5 mmol) and stir at ambient temperature for 1 hr. Adjust to pH=5 with 1M aqueous HCl, extract with EtOAc, dry over anhydrous sodium sulfate, filter, and concentrate under vacuum. Purify by prep-HPLC to give 2-[[6,7-dichloro-1-oxo-10-(1H-pyrazol-4-yl)-3,4-dihydro-2H-pyrazino[1,2-a]indol-9-yl]amino]acetonitrile (1.66 mg, 9%) as a light-yellow solid. ES/MS (m/z): (³⁵Cl/³⁷Cl) 375.1/377.1 (M+H).

### Intermediate 333

### tert-Butyl (2-(6,7-dichloro-4-(cyanomethoxy)-3-(1-(tetrahydro-2H-pyran-2-yl)-1H-pyrazol-4-yl)-1H-indol -1-yl)ethyl)carbamate

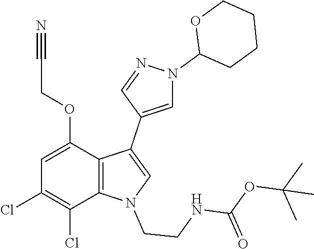 [SMILES: CC(C)(C)OC(=O)NCCn1cc(-c2cnn(C3CCCCO3)c2)c2c(OCC#N)cc(Cl)c(Cl)c21]

Dissolve 2-[[6,7-dichloro-3-(1-tetrahydropyran-2-ylpyrazol-4-yl)-1H-indol-4-yl]oxy]acetonitrile (180 mg, 0.437 mmol) in DMF (4 mL) and add Cs₂CO₃ (285 mg, 0.875 mmol). Stir at ambient temperature for 1 hr. Add tert-butyl 1,2,3-oxathiazolidine-3-carboxylate 2,2-dioxide (147 mg, 0.658 mmol) and stir at ambient temperature for 16 hrs. Quench with 1M aqueous HCl at 0° C. and extract with EtOAc (3×). Wash the combined organic layers with saturated aqueous NaCl (2×), dry over anhydrous sodium sulfate, filter, and concentrate under vacuum. Purify by flash column chromatography eluting with MeOH in DCM to give tert-butyl (2-(6,7-dichloro-4-(cyanomethoxy)-3-(1-(tetrahydro-2H-pyran-2-yl)-1H-pyrazol-4-yl)-1H-indol-1-yl)ethyl)carbamate (220 mg, 99%) as a brown oil. ES/MS (m/z): (³⁵Cl/³⁷Cl) 534.1/536.1 (M+H).

### Intermediate 334

### 2-[1-(2-Aminoethyl)-6,7-dichloro-3-(1H-pyrazol-4-yl)indol-4-yl]oxyacetonitrile

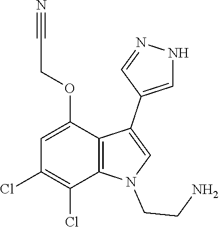 [SMILES: N#CCOc1cc(Cl)c(Cl)c2c1c(-c1cn[nH]c1)cn2CCN]

Dissolve tert-butyl (2-(6,7-dichloro-4-(cyanomethoxy)-3-(1-(tetrahydro-2H-pyran-2-yl)-1H-pyrazol-4-yl)-1H-indol-1-yl)ethyl)carbamate (220 mg, 0.432 mmol) in DCM (2 mL) and add TFA (2 mL). Stir at ambient temperature for 4 hrs. Dilute with water and adjust to pH=8 with saturated aqueous NaHCO₃. Extract with EtOAc (2×). Dry the combined organic layers over anhydrous sodium sulfate, filter, and concentrate under vacuum. Dissolve the residue in THF (4 mL) and water (2 mL). Add LiOH (32 mg, 1.3 mmol) at 0° C. and stir at 0° C. for 1 hr. Dilute with water and extract with EtOAc (3×). Dry the combined organic layers over anhydrous sodium sulfate, filter, and concentrate under vacuum to give 2-[1-(2-aminoethyl)-6,7-dichloro-3-(1H-pyrazol-4-yl)indol-4-yl]oxyacetonitrile (210 mg, 83%) as a yellow oil. ES/MS (m/z): (³⁵Cl/³⁷Cl) 350.0/352.0 (M+H).

### EXAMPLE 136

### N-[2-[6,7-Dichloro-4-(cyanomethoxy)-3-(1H-pyrazol-4-yl)indol-1-yl]ethyl]acetamide

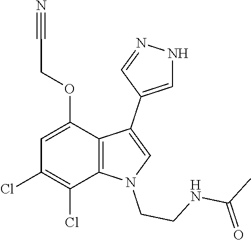 [SMILES: CC(=O)NCCn1cc(-c2cn[nH]c2)c2c(OCC#N)cc(Cl)c(Cl)c21]

Suspend 2-[1-(2-aminoethyl)-6,7-dichloro-3-(1H-pyrazol-4-yl)indol-4-yl]oxyacetonitrile (150 mg, 0.257 mmol) and TEA (80 mg, 0.79 mmol) in DCM (4 mL). Add acetic anhydride (0.035 mL, 0.37 mmol) at 0° C. dropwise and stir at ambient temperature for 1 hr. Combine with another batch run at 0.40×scale for work up and purification. Adjust to pH=8 with saturated aqueous NaHCO₃, extract with EtOAc, wash sequentially with water and saturated aqueous NaCl, dry over anhydrous sodium sulfate, filter, and concentrate under vacuum. Suspend the residue in THF (3 mL) and water (1.5 mL). Add LiOH (19 mg, 0.79 mmol) at 0° C. and stir at 0° C. for 1 hr. Concentrate under vacuum and purify by prep-HPLC to give N-[2-[6,7-dichloro-4-(cyanomethoxy)-3-(1H-pyrazol-4-yl)indol-1-yl]ethyl]acetamide (25.9 mg, 18%) as a white solid. ES/MS (m/z): (³⁵Cl/³⁷Cl) 392.3/394.3 (M+H).

### Intermediate 335

### 6,7-Dichloro-1-(p-tolylsulfonyl)-4-vinyloxy-indole

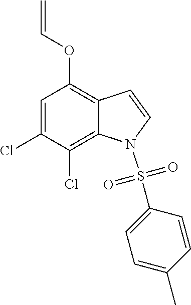 [SMILES: C=COc1cc(Cl)c(Cl)c2c1ccn2S(=O)(=O)c1ccc(C)cc1]

Dissolve 6,7-dichloro-1-(p-tolylsulfonyl)indol-4-ol (3.00 g, 8.00 mmol) in toluene (40 mL). Add vinyl acetate (6.89 g, 80.0 mmol), chloro(1,5-cyclooctadiene)iridium(I) dimer (538 mg, 0.801 mmol), and sodium carbonate (1.02 g, 9.60 mmol). Purge with N₂ and stir at 100° C. for 3 hrs. Dilute with water, extract with EtOAc, wash with saturated aqueous NaCl, dry over anhydrous sodium sulfate, filter, and concentrate under vacuum. Purify by flash column chromatography eluting with EtOAc in petroleum ether to give 6,7-dichloro-1-(p-tolylsulfonyl)-4-vinyloxy-indole (2.98 g, 88%) as a yellow solid.

### Intermediate 336

### Cis-Ethyl 2-[6, 7-dichloro-1-(p-tolylsulfonyl)indol-4-yl]oxycyclopropanecarboxylate

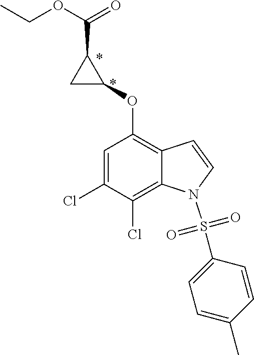 [SMILES: CCOC(=O)[C@@H]1C[C@@H]1Oc1cc(Cl)c(Cl)c2c1ccn2S(=O)(=O)c1ccc(C)cc1]

Dissolve 6,7-dichloro-1-(p-tolylsulfonyl)-4-vinyloxy-indole (2.98 g, 7.02 mmol) in DCM (20 mL) and add rhodium(II) acetate dimer (621 mg, 1.40 mmol). Purge with N₂. Add ethyl diazoacetate (2.81 g, 24.6 mmol) in DCM (20 mL) dropwise and stir at ambient temperature for 1.5 hrs. Concentrate under vacuum and purify by flash column chromatography eluting with EtOAc in petroleum ether to give cis-ethyl 2-[6,7-dichloro-1-(p-tolylsulfonyl)indol-4-yl]oxycyclopropanecarboxylate (550 mg, 16%) as a yellow solid. Material is a racemic mixture. ¹H NMR (DMSO-d6): 7.94 (d, J=3.8 Hz, 1H), 7.74 (d, J=8.3 Hz, 2H), 7.44 (d, J=8.3 Hz, 2H), 7.29 (s, 1H), 6.79 (d, J=3.8 Hz, 1H), 4.41 (ddd, J=7.0, 6.2, 4.6 Hz, 1H), 3.91-3.75 (m, 2H), 2.38 (s, 3H), 2.18 (ddd, J=8.5, 7.0, 6.2 Hz, 1H), 1.57 (ddd, J=7.0, 6.2, 4.6 Hz, 1H), 1.44 (dt, J=8.5, 6.2 Hz, 1H), 0.81 (t, J=7.1 Hz, 3H).

### Intermediate 337

### Cis-Ethyl 2-[(6,7-dichloro-1H-indol-4-yl)oxy]cyclopropane carboxylate

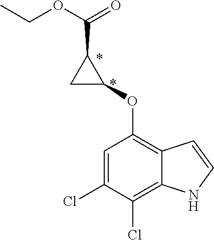 [SMILES: CCOC(=O)[C@@H]1C[C@@H]1Oc1cc(Cl)c(Cl)c2[nH]ccc12]

Dissolve cis-ethyl 2-[6, 7-dichloro-1-(p-tolyl sulfonyl)indol-4-yl]oxycyclopropanecarboxylate (550 mg, 1.12 mmol) in TBAF (6 mL, 1.0M in THF) and stir at ambient temperature for 2 hrs. Dilute with water, extract with EtOAc, wash with aqueous NaCl, dry over anhydrous sodium sulfate, filter, and concentrate under vacuum. Purify by flash column chromatography eluting with EtOAc in petroleum ether to give cis-ethyl 2-[(6,7-dichloro-1H-indol-4-yl)oxy]cyclopropane carboxylate (290 mg, 79%) as a yellow oil. Material is a racemic mixture. ES/MS (m/z): (³⁵Cl/³⁷Cl) 314.0/316.0 (M+H).

### Intermediate 338

### Cis-Ethyl 2-[(6,7-dichloro-3-iodo-1H-indol-4-yl)oxy]cyclopropanecarboxylate

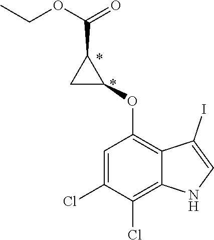 [SMILES: CCOC(=O)[C@@H]1C[C@@H]1Oc1cc(Cl)c(Cl)c2[nH]cc(I)c12]

Dissolve cis-ethyl 2-[(6,7-dichloro-1H-indol-4-yl)oxy]cyclopropane carboxylate (290 mg, 0.877 mmol) in DMF (6 mL) and add NIS (242 mg, 1.05 mmol). Stir at ambient temperature for 1 hr. Dilute with EtOAc, wash with saturated aqueous Na₂SO₃ and saturated aqueous NaCl, dry over anhydrous sodium sulfate, filter, and concentrate under vacuum. Purify by flash column chromatography eluting with EtOAc in petroleum ether to give cis-ethyl 2-[(6,7-dichloro-3-iodo-1H-indol-4-yl)oxy]cyclopropanecarboxylate (380 mg, 94%) as a yellow oil. Material is a racemic mixture.

### Intermediate 339

### Cis-Ethyl 2-[[6,7-dichloro-3-(1-tetrahydropyran-2-ylpyrazol-4-yl)-1H-indol-4-yl]oxy]cyclopropanecarboxylate

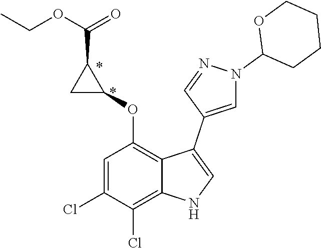 [SMILES: CCOC(=O)[C@@H]1C[C@@H]1Oc1cc(Cl)c(Cl)c2[nH]cc(-c3cnn(C4CCCCO4)c3)c12]

Dissolve cis-ethyl 2-[(6,7-dichloro-3-iodo-1H-indol-4-yl)oxy]cyclopropanecarboxylate (380 mg, 0.820 mmol), 1-(tetrahydro-2H-pyran-2-yl)-4-(4,4,5,5-tetramethyl-1,3,2-dioxaborolan-2-yl)-1H-pyrazole (350 mg, 1.23 mmol), Pd(dtbpf)Cl₂ (55 mg, 0.084 mmol) and sodium carbonate (261 mg, 2.46 mmol) in 1,4-dioxane (8 mL) and water (2 mL). Purge with N₂ and stir at 90° C. for 1 hr. Dilute with water and extract with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give cis-ethyl 2-[[6,7-dichloro-3-(1-tetrahydropyran-2-ylpyrazol-4-yl)-1H-indol-4-yl]oxy]cyclopropanecarboxylate (273 mg, 68%) as a yellow solid. Material is a racemic mixture.

### Intermediate 340

### Cis-[2-[[6,7-Dichloro-3-(1-tetrahydropyran-2-ylpyrazol-4-yl)-1H-indol-4-yl]oxy]cyclopropyl]methanol

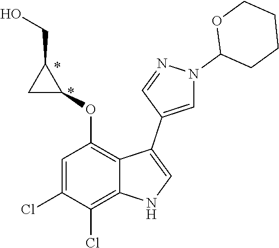 [SMILES: OC[C@@H]1C[C@@H]1Oc1cc(Cl)c(Cl)c2[nH]cc(-c3cnn(C4CCCCO4)c3)c12]

Dissolve cis-ethyl 2-[[6,7-dichloro-3-(1-tetrahydropyran-2-ylpyrazol-4-yl)-1H-indol-4-yl]oxy]cyclopropanecarboxylate (200 mg, 0.409 mmol) in DCM (5 mL). Add DIBAL-H under N₂ atmosphere (1.23 mL, 1.23 mmol, 1.0M in toluene) at 0° C. and stir at 0° C. for 0.5 hr. Quench with water and extract with EtOAc (3×). Wash the combined organic layers with saturated aqueous NaCl, dry over anhydrous sodium sulfate, filter, and concentrate under vacuum to give cis-[2-[[6,7-dichloro-3-(1-tetrahydropyran-2-ylpyrazol-4-yl)-1H-indol-4-yl]oxy]cyclopropyl]methanol (150 mg, 78%) as a yellow solid. Material is a racemic mixture. ES/MS (m/z): (³⁵Cl/³⁷Cl) 422.1/423.8 (M+H).

### EXAMPLE 137

### Cis-[2-[[6,7-Dichloro-3-(1H-pyrazol-4-yl)-1H-indol-4-yl]oxy]cyclopropyl]methanol hydrochloride

### Racemic Mixture

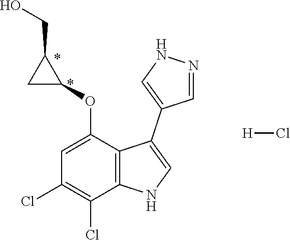 [SMILES: OC[C@@H]1C[C@@H]1Oc1cc(Cl)c(Cl)c2[nH]cc(-c3cn[nH]c3)c12 ; Cl]

Suspend cis-[2-[[6,7-dichloro-3-(1-tetrahydropyran-2-ylpyrazol-4-yl)-1H-indol-4-yl]oxy]cyclopropyl]methanol (200 mg, 0.426 mmol) in THF (3 mL) and add 6M aqueous HCl (4.5 mL). Stir at ambient temperature for 1 hr. and concentrate under vacuum. Purify by prep-HPLC to give racemic cis-[2-[[6,7-dichloro-3-(1H-pyrazol-4-yl)-1H-indol-4-yl]oxy]cyclopropyl]methanol (50.8 mg, 32%, HCl salt) as a white solid. ES/MS (m/z): (³⁵Cl/³⁷Cl) 338.3/340.2 (M+H).

### Intermediate 341

### 2-[6,7-Dichloro-3-(1-tetrahydropyran-2-ylpyrazol-4-yl)indol-1-yl]ethynyl-triisopropyl -silane

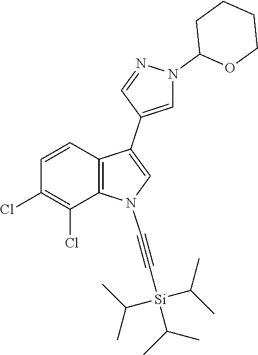 [SMILES: CC(C)[Si](C#Cn1cc(-c2cnn(C3CCCCO3)c2)c2ccc(Cl)c(Cl)c21)(C(C)C)C(C)C]

Dissolve 6,7-dichloro-3-(1-tetrahydropyran-2-ylpyrazol-4-yl)-1H-indole (500 mg, 1.41 mmol) in 1,4-dioxane (6 mL) and add (bromoethynyl)triisopropylsilane (1.90 mg, 7.06 mmol), PEG-400 (80 mg), copper(I) iodide (30 mg, 0.16 mmol) and Cs₂CO₃ (560 mg, 1.72 mmol). Purge with N₂ and stir at 160° C. for 3 hrs. under microwave irradiation. Dilute with water and extract with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give 2-[6,7-dichloro-3-(1-tetrahydropyran-2-ylpyrazol-4-yl)indol-1-yl]ethynyl-triisopropyl-silane (300 mg, 39%) as a brown oil.

### Intermediate 342

### 6,7-Dichloro-1-ethynyl-3-(1-tetrahydropyran-2-ylpyrazol-4-yl)indole

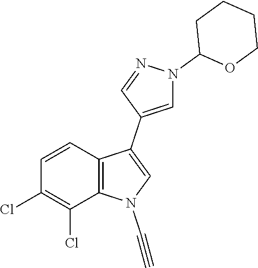 [SMILES: C#Cn1cc(-c2cnn(C3CCCCO3)c2)c2ccc(Cl)c(Cl)c21]

Dissolve 2-[6,7-dichloro-3-(1-tetrahydropyran-2-ylpyrazol-4-yl)indol-1-yl]ethynyl-triisopropyl-silane (300 mg, 0.552 mmol) in THF (8 mL) and add TBAF (4 mL, 1.0M in THF). Stir at 0° C. for 0.5 hr. Dilute with water and extract with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give 6,7-dichloro-1-ethynyl-3-(1-tetrahydropyran-2-ylpyrazol-4-yl)indole (180 mg, 86%) as a brown solid.

### Intermediate 343

### tert-Butyl-[2-[4-[6, 7-dichloro-3-(1-tetrahydropyran-2-ylpyrazol -4-yl)indol -1-yl]triazol -1-yl]ethoxy]-dimethyl-silane

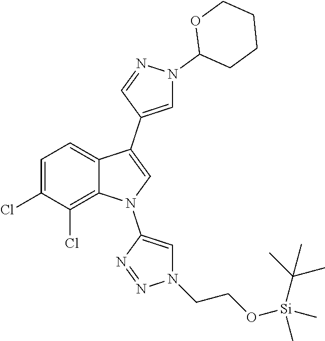 [SMILES: CC(C)(C)[Si](C)(C)OCCn1cc(-n2cc(-c3cnn(C4CCCCO4)c3)c3ccc(Cl)c(Cl)c32)nn1]

Dissolve 6,7-dichloro-1-ethynyl-3-(1-tetrahydropyran-2-ylpyrazol -4-yl)indole (180 mg, 0.475 mmol) in DMF (3 mL) and water (3 mL). Add copper (6 mg, 0.09 mmol), cupric sulfate (16 mg, 0.10 mmol) and (2-azidoethoxy)(tert-butyl)dimethylsilane (130 mg, 0.613 mmol). Purge with N₂ and stir at 110° C. for 0.5 hr. Dilute with water and extract with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give tert-butyl-[2-[4-[6,7-dichloro-3-(1-tetrahydropyran-2-ylpyrazol-4-yl)indol-1-yl]triazol-1-yl]ethoxy]-dimethyl-silane (170 mg, 61%) as a yellow oil.

### EXAMPLE 138

### 2-[4-[6, 7-Dichloro-3-(1H-pyrazol-4-yl)indol-1-yl]triazol-1-yl]ethanol

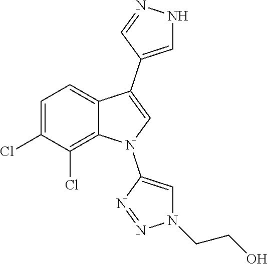 [SMILES: OCCn1cc(-n2cc(-c3cn[nH]c3)c3ccc(Cl)c(Cl)c32)nn1]

Suspend tert-butyl-[2-[4-[6,7-dichloro-3-(1-tetrahydropyran-2-ylpyrazol-4-yl)indol-1-yl]triazol-1-yl]ethoxy]-dimethyl-silane (140 mg, 0.237 mmol) in THF (1.5 mL) and add 6M aqueous HCl (1.5 mL). Stir at ambient temperature for 1 hr. and concentrate under vacuum. Purify by prep-HPLC to give 2-[4-[6,7-dichloro-3-(1H-pyrazol-4-yl)indol-1-yl]triazol-1-yl]ethanol (58.7 mg, 67%) as a white solid. ES/MS (m/z): (³⁵Cl/³⁷Cl) 363.3/365.2 (M+H).

### Intermediate 344

### tert-Butyl-[3-[4-[6, 7-dichloro-3-(1-tetrahydropyran-2-ylpyrazol-4-yl)indol -1-yl]triazol-1-yl]propoxy]-dimethyl-silane

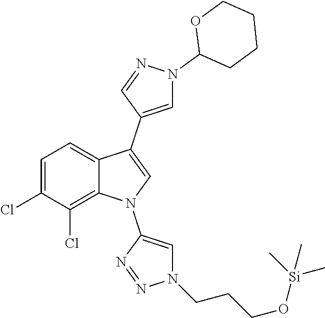 [SMILES: C[Si](C)(C)OCCCn1cc(-n2cc(-c3cnn(C4CCCCO4)c3)c3ccc(Cl)c(Cl)c32)nn1]

Dissolve 6,7-dichloro-1-ethynyl-3-(1-tetrahydropyran-2-ylpyrazol-4-yl)indole (70 mg, 0.19 mmol) in DMF (2 mL) and water (2 mL). Add copper (4 mg, 0.06 mmol), cupric sulfate (7 mg, 0.04 mmol) and (3-azidopropoxy)(tert-butyl)dimethylsilane (CAS:142288-01-5, 52 mg, 0.23 mmol). Purge with N₂ and stir at 110° C. for 0.5 hr. Combine with another batch run at 0.43×scale for work up and purification. Dilute with water and extract with EtOAc (3×). Wash the combined organic layers with saturated aqueous NaCl, dry over anhydrous sodium sulfate, filter, and concentrate under vacuum to give tert-butyl-[3-[4-[6,7-dichloro-3-(1-tetrahydropyran-2-ylpyrazol-4-yl)indol-1-yl]triazol-1-yl]propoxy]-dimethyl-silane (105 mg, 53%) as a black oil.

### EXAMPLE 139

### 3-[4-[6,7-Dichloro-3-(1H-pyrazol-4-yl)indol-1-yl]triazol-1-yl]propan-1-ol

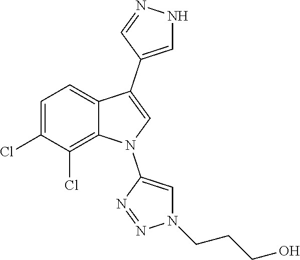 [SMILES: OCCCn1cc(-n2cc(-c3cn[nH]c3)c3ccc(Cl)c(Cl)c32)nn1]

Suspend tert-butyl-[3-[4-[6,7-dichloro-3-(1-tetrahydropyran-2-ylpyrazol-4-yl)indol-1-yl]triazol-1-yl]propoxy]-dimethyl-silane (105 mg, 0.146 mmol) in THF (2 mL) and add 6M aqueous HCl (2 mL). Stir at ambient temperature for 1 hr. and concentrate under vacuum. Purify by prep-HPLC to give 3-[4-[6,7-dichloro-3-(1H-pyrazol-4-yl)indol-1-yl]triazol-1-yl]propan-1-ol (45.1 mg, 82%) as a white solid. ES/MS (m/z): (³⁵Cl/³⁷Cl) 377.2/379.2 (M+H).

### Intermediate 345

### 6,7-Dichloro-1-[1-[(2,2-dimethyl-1,3-dioxolan-4-yl)methyl]triazol-4-yl]-3-(1-tetrahydropyran-2-ylpyrazol-4-yl)indole

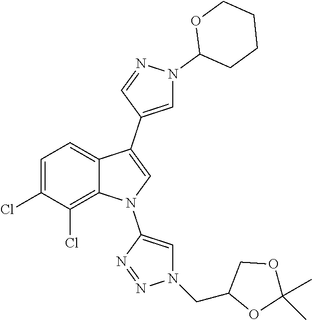 [SMILES: CC1(C)OCC(Cn2cc(-n3cc(-c4cnn(C5CCCCO5)c4)c4ccc(Cl)c(Cl)c43)nn2)O1]

Dissolve 6,7-dichloro-1-ethynyl-3-(1-tetrahydropyran-2-ylpyrazol-4-yl)indole (90 mg, 0.24 mmol) in DMF (2 mL) and water (3 mL). Add copper (3 mg, 0.05 mmol), cupric sulfate (8 mg, 0.05 mmol) and 4-(azidomethyl)-2,2-dimethyl-1,3-dioxolane (CAS: 25261-56-7, 150 mg, 0.286 mmol). Purge with N₂ and stir at 110° C. for 0.5 hr. Dilute with water and extract with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give 6,7-dichloro-1-[1-[(2,2-dimethyl-1,3-dioxolan-4-yl)methyl]triazol-4-yl]-3-(1-tetrahydropyran-2-ylpyrazol-4-yl)indole (120 mg, 66%) as a yellow oil. ES/MS (m/z): (³⁵Cl/³⁷Cl) 517.0/519.1 (M+H).

### EXAMPLE 140

### 3-[4-[6,7-Dichloro-3-(1H-pyrazol-4-yl)indol-1-yl]triazol-1-yl]propane-1,2-diol

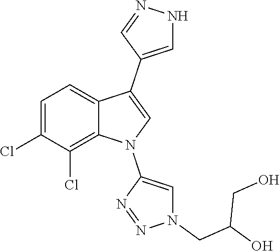 [SMILES: OCC(O)Cn1cc(-n2cc(-c3cn[nH]c3)c3ccc(Cl)c(Cl)c32)nn1]

Suspend 6,7-dichloro-1-[1-[(2,2-dimethyl-1,3-dioxolan-4-yl)methyl]triazol-4-yl]-3-(1-tetrahydropyran-2-ylpyrazol-4-yl)indole (120 mg, 0.158 mmol) in THF (2 mL) and add 6M aqueous HCl (2 mL). Stir at ambient temperature for 1 hr. and concentrate under vacuum. Purify by prep-HPLC to give 3-[4-[6,7-dichloro-3-(1H-pyrazol-4-yl)indol-1-yl]triazol-1-yl]propane-1,2-diol (42.2 mg, 68%) as a white solid. ES/MS (m/z): (³⁵Cl/³⁷Cl) 393.3/395.2 (M+H).

### Intermediate 346

### Methyl 3-[[3,4-dichloro-10-(1-tetrahydropyran-2-ylpyrazol-4-yl)-6,7,8,9-tetrahydropyrido[1,2-a]indol-7-yl]amino]-3-oxo-propanoate

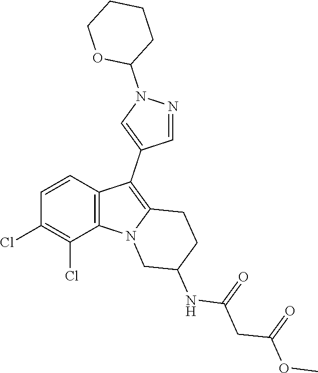 [SMILES: COC(=O)CC(=O)NC1CCc2c(-c3cnn(C4CCCCO4)c3)c3ccc(Cl)c(Cl)c3n2C1]

Dissolve 3,4-dichloro-10-(1-tetrahydropyran-2-ylpyrazol-4-yl)-6,7,8,9-tetrahydropyrido[1,2-a]indol-7-amine (100 mg, 0.222 mmol) and TEA (68 mg, 0.67 mmol) in DCM (3 mL). Add methyl 3-chloro-3-oxo-propanoate (37 mg, 0.27 mmol) at 0° C. and stir at 0° C. for 1.5 hrs. Dilute with water and extract with DCM (3×). Wash the combined organic layers with saturated aqueous NaCl, dry over anhydrous sodium sulfate, filter, and concentrate under vacuum. Purify by flash column chromatography eluting with MeOH in DCM to give methyl 3-[[3,4-dichloro-10-(1-tetrahydropyran-2-ylpyrazol-4-yl)-6,7,8,9-tetrahydropyrido[1,2-a]indol-7-yl]amino]-3-oxo-propanoate (120 mg, 96%) as a yellow oil. ES/MS (m/z): (³⁵Cl/³⁷Cl) 505.1/507.1 (M+H).

### Intermediate 347

### N′-[3,4-Dichloro-10-(1-tetrahydropyran-2-ylpyrazol-4-yl)-6,7, 8,9-tetrahydropyrido[1,2-a]indol-7-yl]-N-methyl-propanediamide

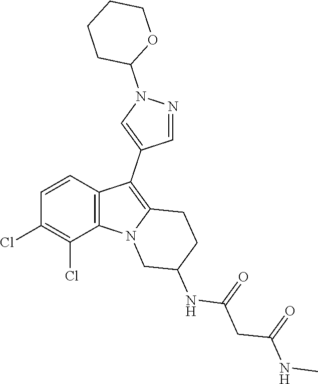 [SMILES: CNC(=O)CC(=O)NC1CCc2c(-c3cnn(C4CCCCO4)c3)c3ccc(Cl)c(Cl)c3n2C1]

Dissolve methyl 3-[[3,4-dichloro-10-(1-tetrahydropyran-2-ylpyrazol-4-yl)-6,7,8,9-tetrahydropyrido[1,2-a]indol-7-yl]amino]-3-oxo-propanoate (120 mg, 0.214 mmol) in methylamine (3.53 mL, 7.06 mmol, 2M in THF). Stir at ambient temperature for 16 hrs., and concentrate under vacuum to give N′-[3,4-dichloro-10-(1-tetrahydropyran-2-ylpyrazol-4-yl)-6,7,8,9-tetrahydropyrido[1,2-a]indol-7-yl]-N-methyl-propanediamide (160 mg, 89%) as a yellow oil. ES/MS (m/z): (³⁵Cl/³⁷Cl) 504.2/506.2 (M+H) and 420.1/422.1 (M+H-THP).

### EXAMPLE 141

### N′-[3,4-Dichloro-10-(1H-pyrazol-4-yl)-6,7,8,9-tetrahydropyrido[1,2-a]indol-7-yl]-N-methyl-propanediamide

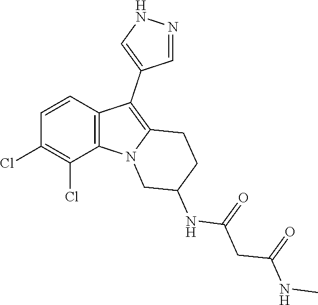 [SMILES: CNC(=O)CC(=O)NC1CCc2c(-c3cn[nH]c3)c3ccc(Cl)c(Cl)c3n2C1]

Suspend N′-[3,4-dichloro-10-(1-tetrahydropyran-2-ylpyrazol-4-yl)-6,7,8,9-tetrahydropyrido[1,2-a]indol-7-yl]-N-methyl-propanediamide (150 mg, 0.297 mmol) in DCM (2 mL) and add TFA (2 mL). Stir at ambient temperature for 2 hrs and concentrate under vacuum. Purify by prep-HPLC to give N′-[3,4-dichloro-10-(1H-pyrazol-4-yl)-6,7,8,9-tetrahydropyrido[1,2-a]indol-7-yl]-N-methyl-propanediamide (22.6 mg, 18%) as a white solid. ES/MS (m/z): (³⁵Cl/³⁷Cl) 420.3/422.3 (M+H).

### Intermediate 348

### 9-Bromo-6,7-dichloro-2-(2-tetrahydropyran-2-yloxy ethyl)-3,4-dihydropyrazino[1,2-a]indol-1-one

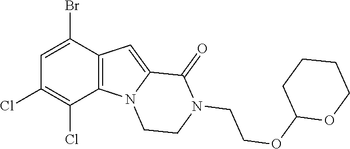 [SMILES: O=C1c2cc3c(Br)cc(Cl)c(Cl)c3n2CCN1CCOC1CCCCO1]

Dissolve 9-bromo-6, 7-dichloro-3,4-dihydropyrazino[1, 2-a]indol-1(2H)-one (500 mg, 1.42 mmol) in DMF (8 mL). Add sodium hydride (120 mg, 3.00 mmol, 60% dispersion in mineral oil) at 0° C. and stir for 0.5 hr. Add 2-(2-bromoethoxy)tetrahydro-2H-pyran (620 mg, 2.85 mmol) dropwise at 0° C. Stir at 0° C. for 2 hrs. Dilute with water and extract with EtOAc (3×). Wash the combined organic layers with 4% aqueous LiCl (3×), dry over anhydrous sodium sulfate, filter, and concentrate under vacuum. Purify by flash column chromatography eluting with EtOAc in petroleum ether to give 9-bromo-6,7-dichloro-2-(2-tetrahydropyran-2-yloxyethyl)-3,4-dihydropyrazino[1,2-a]indol-1-one (551 mg, 75%) as a colorless oil.

### Intermediate 349

### 6,7-Dichloro-9-hydroxy-2-(2-tetrahydropyran-2-yloxy ethyl)-3,4-dihydropyrazino[1,2-a]indol-1-one

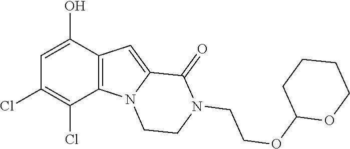 [SMILES: O=C1c2cc3c(O)cc(Cl)c(Cl)c3n2CCN1CCOC1CCCCO1]

Dissolve 9-bromo-6,7-dichloro-2-(2-tetrahydropyran-2-yloxyethyl)-3,4-dihydropyrazino[1,2-a]indol-1-one (550 mg, 1.07 mmol) in 1,4-dioxane (10 mL). Add t-BuONa (260 mg, 2.62 mmol), water (5 mL), and t-BuBrettPhos-Pd-G₃ (50 mg, 0.057 mmol) at ambient temperature. Purge with N₂ and stir at 65° C. for 3 hrs. Quench with saturated aqueous NH₄Cl. Add 1M aqueous HCl to adjust to pH=5 and extract with EtOAc (2×). Wash the combined organic layers with saturated aqueous NaCl (2×), dry over anhydrous sodium sulfate, filter, and concentrate under vacuum. Purify by flash column chromatography eluting with EtOAc in petroleum ether to give 6,7-dichloro-9-hydroxy-2-(2-tetrahydropyran-2-yloxyethyl)-3,4-dihydropyrazino[1,2-a]indol-1-one (190 mg, 42%) as a yellow solid.

### Intermediate 350

### 2-[[6,7-Dichloro-1-oxo-2-(2-tetrahydropyran-2-yloxyethyl)-3,4-dihydropyrazino[1,2-a]indol-9-yl]oxy]acetonitrile

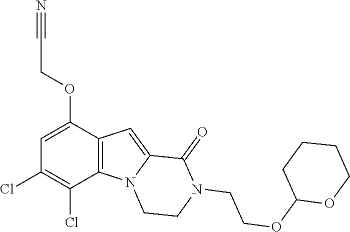 [SMILES: N#CCOc1cc(Cl)c(Cl)c2c1cc1n2CCN(CCOC2CCCCO2)C1=O]

Dissolve 6,7-dichloro-9-hydroxy-2-(2-tetrahydropyran-2-yloxy ethyl)-3,4-dihydropyrazino[1,2-a]indol-1-one (190 mg, 0.452 mmol) and potassium carbonate (70 mg, 0.51 mmol) in DMF (3 mL). Add bromoacetonitrile (0.070 mL, 1.0 mmol) dropwise at 0° C. Stir at 0° C. for 2 hrs. then at ambient temperature for 1 hr. Dilute with water and extract with EtOAc (3×). Wash the combined organic layers with 4% aqueous LiCl (3×), dry over anhydrous sodium sulfate, filter, and concentrate under vacuum. Purify by flash column chromatography eluting with EtOAc in petroleum ether to give 2-[[6,7-dichloro-1-oxo-2-(2-tetrahydropyran-2-yl oxy ethyl)-3,4-dihydropyrazino[1,2-a]indol-9-yl]oxy]acetonitrile (165 mg, 75%) as a yellow solid.

### Intermediate 351

### 2-[[10-Bromo-6,7-dichloro-1-oxo-2-(2-tetrahydropyran-2-yloxyethyl)-3,4-dihydropyrazino[1,2-a]indol-9-yl]oxy]acetonitrile

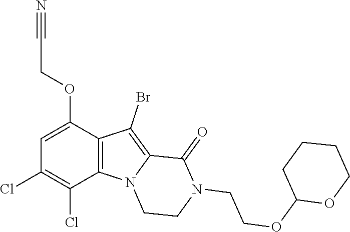 [SMILES: N#CCOc1cc(Cl)c(Cl)c2c1c(Br)c1n2CCN(CCOC2CCCCO2)C1=O]

Dissolve 2-[[6,7-dichloro-1-oxo-2-(2-tetrahydropyran-2-yloxyethyl)-3,4-dihydropyrazino[1,2-a]indol-9-yl]oxy]acetonitrile (165 mg, 0.339 mmol) in DMF (3 mL). Add NBS (60 mg, 0.34 mmol) at 0° C., then stir at ambient temperature for 2 hrs. Dilute with water and extract with EtOAc (3×). Wash the combined organic layers with 4% aqueous LiCl (3×), dry over anhydrous sodium sulfate, filter, and concentrate under vacuum to give 2-[[10-bromo-6,7-dichloro-1-oxo-2-(2-tetrahydropyran-2-yloxyethyl)-3,4-dihydropyrazino[1,2-a]indol-9-yl]oxy]acetonitrile (190 mg, 62%) as a yellow solid. ES/MS (m/z): 431.9/433.9/435.9 (M+H-THP).

### Intermediate 352

### 2-[[6,7-Dichloro-1-oxo-2-(2-tetrahydropyran-2-yloxy ethyl)-10-(1-tetrahydropyran-2-ylpyrazol-4-yl)-3,4-dihydropyrazino[1,2-a]indol-9-yl]oxy]acetonitrile

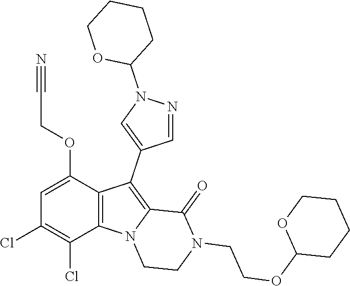 [SMILES: N#CCOc1cc(Cl)c(Cl)c2c1c(-c1cnn(C3CCCCO3)c1)c1n2CCN(CCOC2CCCCO2)C1=O]

Suspend 2-[[10-bromo-6,7-dichloro-1-oxo-2-(2-tetrahydropyran-2-yloxyethyl)-3,4-dihydropyrazino[1,2-a]indol-9-yl]oxy]acetonitrile (190 mg, 0.211 mmol), 1-(tetrahydro-2H-pyran-2-yl)-4-(4,4,5,5-tetramethyl-1,3,2-dioxaborolan-2-yl)-1H-pyrazole (120 mg, 0.410 mmol), Pd(dppf)Cl₂ (20 mg, 0.026 mmol) and K₂CO₃ (45 mg, 0.45 mmol) in 1,4-dioxane (2 mL) and water (0.5 mL). Purge with N₂ and stir at 90° C. for 2 hrs. Cool to ambient temperature, quench with water, extract with EtOAc, dry over anhydrous sodium sulfate, filter, and concentrate under vacuum. Purify by flash column chromatography eluting with EtOAc in petroleum ether to give 2-[[6,7-dichloro-1-oxo-2-(2-tetrahydropyran-2-yloxyethyl)-10-(1-tetrahydropyran-2-ylpyrazol-4-yl)-3,4-dihydropyrazino[1,2-a]indol-9-yl]oxy]acetonitrile (100 mg, 42%) as a yellow solid. ES/MS (m/z): (³⁵Cl/³⁷Cl) 588.1/590.1 (M+H).

### EXAMPLE 142

### 2-[[6,7-Dichloro-2-(2-hydroxyethyl)-1-oxo-10-(1H-pyrazol-4-yl)-3,4-dihydropyrazino[1,2-a]indol-9-yl]oxy]acetonitrile

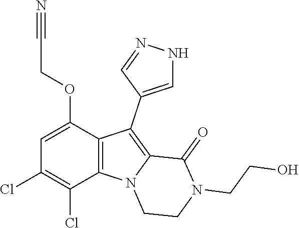 [SMILES: N#CCOc1cc(Cl)c(Cl)c2c1c(-c1cn[nH]c1)c1n2CCN(CCO)C1=O]

Suspend 2-[[6,7-dichloro-1-oxo-2-(2-tetrahydropyran-2-yloxyethyl)-10-(1-tetrahydropyran-2-ylpyrazol-4-yl)-3,4-dihydropyrazino[1,2-a]indol-9-yl]oxy]acetonitrile (100 mg, 0.088 mmol) in DCM (1.5 mL). Add TFA (0.5 mL) and stir at ambient temperature for 2 hrs. Concentrate under vacuum and dissolve the residue in THF (2 mL), then add 2N aqueous LiOH (2 mL) and stir at ambient temperature for 1 hr. Quench with water, extract with EtOAc, dry over anhydrous sodium sulfate, filter, and concentrate under vacuum. Purify by reverse phase prep-HPLC to give 2-[[6,7-dichloro-2-(2-hydroxyethyl)-1-oxo-10-(1H-pyrazol-4-yl)-3,4-dihydropyrazino[1,2-a]indol-9-yl]oxy]acetonitrile (5.80 mg, 16%) as a white solid. ES/MS (m/z): (³⁵Cl/³⁷Cl) 420.2/422.2 (M+H).

### Intermediate 353

### 9-Bromo-2-[2-[tert-butyl(dimethyl)silyl]oxyethyl]-6, 7-dichloro-3,4-dihydropyrazino[1,2-a]indol-1-one

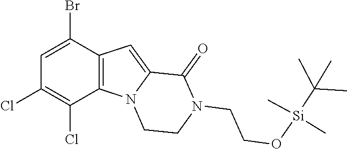 [SMILES: CC(C)(C)[Si](C)(C)OCCN1CCn2c(cc3c(Br)cc(Cl)c(Cl)c32)C1=O]

Dissolve 9-bromo-6,7-dichloro-3,4-dihydro-2H-pyrazino[1,2-a]indol-1-one (700 mg, 1.99 mmol) in DMF (10 mL). Add sodium hydride (160 mg, 4.00 mmol, 60% dispersion in mineral oil) at 0° C. and stir at 0° C. for 0.5 hr. Add tert-butyl(2-iodoethoxy)dimethylsilane (1.2 g, 4.0 mmol) at 0° C. Stir at 0° C. for 2 hrs., and then at ambient temperature for 16-18 hrs. Dilute with water and filter the precipitate, wash with water (3×), and dry under vacuum to give 9-bromo-2-[2-[tert-butyl(dimethyl)silyl]oxyethyl]-6,7-dichloro-3,4-dihydropyrazino[1,2-a]indol-1-one (880 mg, 85%) as a yellow solid.

### Intermediate 354

### tert-Butyl N-[2-[2-[tert-butyl(dimethyl)silyl]oxyethyl]-6,7-dichloro-1-oxo-3,4-dihydropyrazino[1,2-a]indol-9-yl]carbamate

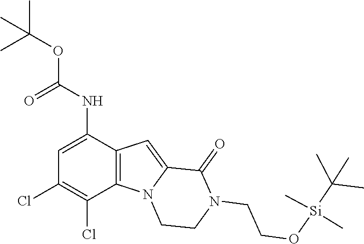 [SMILES: CC(C)(C)OC(=O)Nc1cc(Cl)c(Cl)c2c1cc1n2CCN(CCO[Si](C)(C)C(C)(C)C)C1=O]

Dissolve 9-bromo-2-[2-[tert-butyl(dimethyl)silyl]oxyethyl]-6, 7-di chloro-3,4-dihydropyrazino[1,2-a]indol-1-one (440 mg, 0.849 mmol) and tert-butyl carbamate (220 mg, 1.88 mmol) in 1,4-dioxane (10 mL). Add Pd₂(dba)₃ (80 mg, 0.087 mmol), XantPhos (77 mg, 0.13 mmol) and Cs₂CO₃ (550 mg, 1.69 mmol). Purge with N₂ and stir at 100° C. for 16-18 hrs. Quench with water, extract with EtOAc, dry over anhydrous sodium sulfate, filter, and concentrate under vacuum. Purify by flash column chromatography eluting with EtOAc in petroleum ether to give tert-butyl N-[2-[2-[tert-butyl(dimethyl)silyl]oxyethyl]-6,7-dichloro-1-oxo-3,4-dihydropyrazino[1,2-a]indol-9-yl]carbamate (302 mg, 64%) as a yellow solid.

### Intermediate 355

### tert-Butyl N-[2-[2-[tert-butyl(dimethyl)silyl]oxyethyl]-6,7-dichloro-1-oxo-3,4-dihydropyrazino[1,2-a]indol-9-yl]-N-(cyanomethyl)carbamate

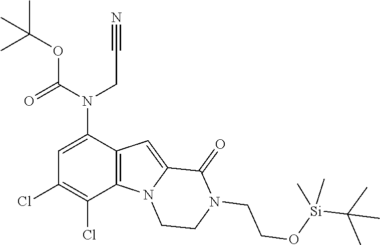 [SMILES: CC(C)(C)OC(=O)N(CC#N)c1cc(Cl)c(Cl)c2c1cc1n2CCN(CCO[Si](C)(C)C(C)(C)C)C1=O]

Dissolve tert-butyl N-[2-[2-[tert-butyl(dimethyl)silyl]oxyethyl]-6,7-dichloro-1-oxo-3,4-dihydropyrazino[1,2-a]indol-9-yl]carbamate (300 mg, 0.539 mmol) in DMF (5 mL). Add sodium hydride (40 mg, 1.0 mmol, 60% dispersion in mineral oil) at 0° C. and stir for 0.5 hr. Add bromoacetonitrile (0.1 mL, 1.4 mmol) dropwise at 0° C. and stir at ambient temperature for 1 hr. Dilute with water and extract with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give tert-butyl N-[2-[2-[tert-butyl(dimethyl)silyl]oxyethyl]-6,7-dichloro-1-oxo-3,4-dihydropyrazino[1,2-a]indol-9-yl]-N-(cyanomethyl)carbamate (220 mg, 68%) as a yellow oil.

### Intermediate 356

### tert-Butyl N-[10-bromo-2-[2-[tert-butyl(dimethyl)silyl]oxyethyl]-6,7-dichloro-1-oxo-3,4-dihydropyrazino[1,2-a]indol-9-yl]-N-(cyanomethyl)carbamate

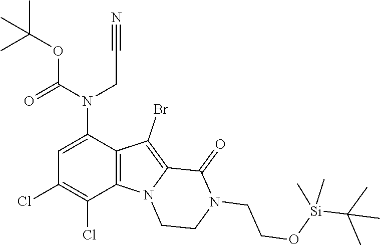 [SMILES: CC(C)(C)OC(=O)N(CC#N)c1cc(Cl)c(Cl)c2c1c(Br)c1n2CCN(CCO[Si](C)(C)C(C)(C)C)C1=O]

Dissolve tert-butyl N-[2-[2-[tert-butyl(dimethyl)silyl]oxyethyl]-6,7-dichloro-1-oxo-3,4-dihydropyrazino[1,2-a]indol-9-yl]-N-(cyanomethyl)carbamate (220 mg, 0.368 mmol) in DMF (3 mL). Add NBS (100 mg, 0.562 mmol) and stir at ambient temperature for 2 hrs. Dilute with water and extract with EtOAc (3×). Wash the combined organic layers with 4% aqueous LiCl (3×), dry over anhydrous sodium sulfate, filter, and concentrate under vacuum. Purify by flash column chromatography eluting with EtOAc in petroleum ether to give tert-butyl N-[10-bromo-2-[2-[tert-butyl(dimethyl)silyl]oxyethyl]-6,7-dichloro-1-oxo-3,4-dihydropyrazino[1,2-a]indol-9-yl]-N-(cyanomethyl)carbamate (80 mg, 32%) as a yellow gum.

### Intermediate 357

### tert-Butyl N-[2-[2-[tert-butyl(dimethyl)silyl]oxyethyl]-6,7-dichloro-1-oxo-10-(1-tetrahydropyran-2-ylpyrazol-4-yl)-3,4-dihydropyrazino[1,2-a]indol-9-yl]-N-(cyanomethyl)carbamate

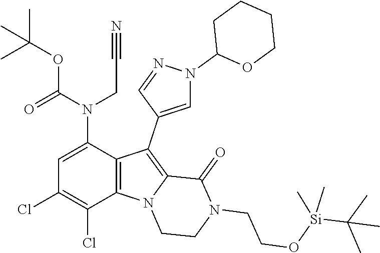 [SMILES: CC(C)(C)OC(=O)N(CC#N)c1cc(Cl)c(Cl)c2c1c(-c1cnn(C3CCCCO3)c1)c1n2CCN(CCO[Si](C)(C)C(C)(C)C)C1=O]

Suspend tert-butyl N-[10-bromo-2-[2-[tert-butyl(dimethyl)silyl]oxyethyl]-6,7-dichloro-1-oxo-3,4-dihydropyrazino[1,2-a]indol-9-yl]-N-(cyanomethyl)carbamate (80 mg, 0.12 mmol), 1-(tetrahydro-2H-pyran-2-yl)-4-(4,4,5,5-tetramethyl-1,3,2-dioxaborolan-2-yl)-1H-pyrazole (70 mg, 0.24 mmol), K₂CO₃ (25 mg, 0.25 mmol) and Pd(dppf)Cl₂ (10 mg, 0.013 mmol) in 1,4-dioxane (4 mL) and water (1 mL). Purge with N₂ and heat at 90° C. for 2 hrs. Cool to ambient temperature, dilute with water, and extract with EtOAc (3×). Dry the combined organic layers over anhydrous sodium sulfate, filter, and concentrate under vacuum. Purify by flash column chromatography eluting with EtOAc in petroleum ether to give tert-butyl N-[2-[2-[tert-butyl(dimethyl)silyl]oxyethyl]-6,7-dichloro-1-oxo-10-(1-tetrahydropyran-2-ylpyrazol-4-yl)-3,4-dihydropyrazino[1,2-a]indol-9-yl]-N-(cyanomethyl)carbamate (34 mg, 29%) as a yellow gum. ES/MS (m/z): (³⁵Cl/³⁷Cl) 717.2/718.9 (M+H).

### EXAMPLE 143

### 2-[[6,7-Dichloro-2-(2-hydroxyethyl)-1-oxo-10-(1H-pyrazol-4-yl)-3,4-dihydropyrazino[1,2-a]indol-9-yl]amino]acetonitrile

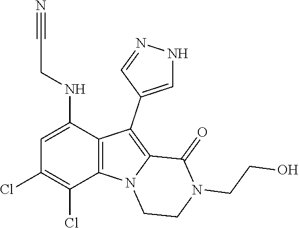 [SMILES: N#CCNc1cc(Cl)c(Cl)c2c1c(-c1cn[nH]c1)c1n2CCN(CCO)C1=O]

Suspend tert-butyl N-[2-[2-[tert-butyl(dimethyl)silyl]oxyethyl]-6,7-dichloro-1-oxo-10-(1-tetrahydropyran-2-ylpyrazol-4-yl)-3,4-dihydropyrazino[1,2-a]indol-9-yl]-N-(cyanomethyl)carbamate (30 mg, 0.030 mmol) in DCM (1 mL). Add TFA (0.5 mL) and stir at ambient temperature for 2 hrs. Concentrate under vacuum and dissolve the residue in THF (1 mL), then add a solution of LiOH (100 mg, 4.18 mmol) in water (0.5 mL) and stir at ambient temperature for 1 hr. Add 1M HCl to adjust the pH to 3, extract with EtOAc, dry over anhydrous sodium sulfate, filter, and concentrate under vacuum. Purify by prep-HPLC to give 2-[[6,7-dichloro-2-(2-hydroxyethyl)-1-oxo-10-(1H-pyrazol-4-yl)-3,4-dihydropyrazino[1,2-a]indol-9-yl]amino]acetonitrile (4.51 mg, 33%) as an off-white solid. ES/MS (m/z): (³⁵Cl/³⁷Cl) 419.3/421.2 (M+H).

### Intermediate 358

### N-[3-[tert-Butyl(dimethyl)silyl]oxypropyl]-6,7-dichloro-3-(1-tetrahydropyran-2-ylpyrazol-4-yl)-1H-indol-4-amine

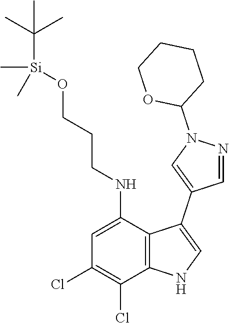 [SMILES: CC(C)(C)[Si](C)(C)OCCCNc1cc(Cl)c(Cl)c2[nH]cc(-c3cnn(C4CCCCO4)c3)c12]

Dissolve 4-bromo-6,7-dichloro-1-(p-tolylsulfonyl)-3-(1-tetrahydropyran-2-ylpyrazol-4-yl)indole (100 mg, 0.158 mmol), 3-(tert-butyldimethylsilyloxy)propan-1-amine (50 mg, 0.25 mmol), Pd(t-Bu₃P)₂ (12 mg, 0.023 mmol) and sodium tert-butoxide (47 mg, 0.47 mmol) in toluene (3 mL). Purge with N₂ and stir at 100° C. under a N₂ atmosphere for 16-18 hrs. Concentrate under vacuum, dilute with water, extract with EtOAc, dry over anhydrous sodium sulfate, filter, and concentrate under vacuum. Purify by flash column chromatography eluting with EtOAc in petroleum ether to give N-[3-[tert-butyl(dimethyl)silyl]oxypropyl]-6,7-dichloro-3-(1-tetrahydropyran-2-ylpyrazol-4-yl)-1H-indol-4-amine (60 mg, 50%) as a yellow solid. ES/MS (m/z): (³⁵Cl/³⁷Cl) 523.2/525.2 (M+H).

### EXAMPLE 144

### 3-[[6,7-Dichloro-3-(1H-pyrazol-4-yl)-1H-indol-4-yl]amino]propan-1-ol

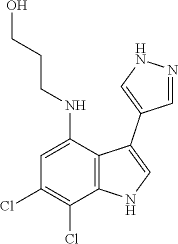 [SMILES: OCCCNc1cc(Cl)c(Cl)c2[nH]cc(-c3cn[nH]c3)c12]

Dissolve N-[3-[tert-butyl(dimethyl)silyl]oxypropyl]-6,7-dichloro-3-(1-tetrahydropyran-2-ylpyrazol-4-yl)-1H-indol-4-amine (60 mg, 0.079 mmol) in 6M aqueous HCl (4 mL) and stir at ambient temperature for 2 hrs. Purify by prep-HPLC to give 3-[[6,7-dichloro-3-(1H-pyrazol-4-yl)-1H-indol-4-yl]amino]propan-1-ol (8.36 mg, 32%) as a white solid. ES/MS (m/z): (³⁵Cl/³⁷Cl) 325.3/327.2 (M+H).

### Intermediate 359

### tert-Butyl 9-(tert-butoxycarbonylamino)-6,7-dichloro-10-iodo-3,4-dihydro-1H-pyrazino[1,2-a]indole-2-carboxylate

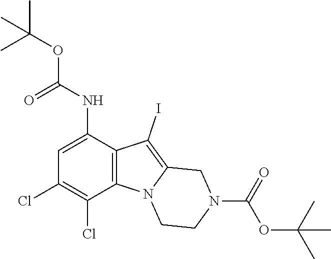 [SMILES: CC(C)(C)OC(=O)Nc1cc(Cl)c(Cl)c2c1c(I)c1n2CCN(C(=O)OC(C)(C)C)C1]

Dissolve tert-butyl 9-(tert-butoxycarbonylamino)-6, 7-dichloro-3,4-dihydro-1H-pyrazino[1,2-a]indole-2-carboxylate (1.50 g, 3.29 mmol) in DMF (10 mL). Add NIS (640 mg, 2.84 mmol) and stir at ambient temperature for 2 hrs. Dilute with water and collect the solids by filtration to give tert-butyl 9-(tert-butoxycarbonylamino)-6,7-dichloro-10-iodo-3,4-dihydro-1H-pyrazino[1,2-a]indole-2-carboxylate (1.30 g, 68%) as a yellow solid. ES/MS (m/z): (³⁵Cl/³⁷Cl) 580.4/582.4 (M−H) and 526.0/528.0 (M+H-tBu).

### Intermediate 360

### tert-Butyl 9-(tert-butoxycarbonylamino)-6, 7-dichloro-10-(1-tetrahydropyran-2-ylpyrazol-4-yl)-3,4-dihydro-1H-pyrazino[1,2-a]indole-2-carboxylate

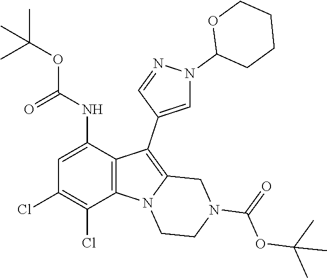 [SMILES: CC(C)(C)OC(=O)Nc1cc(Cl)c(Cl)c2c1c(-c1cnn(C3CCCCO3)c1)c1n2CCN(C(=O)OC(C)(C)C)C1]

Dissolve tert-butyl 9-(tert-butoxycarbonylamino)-6,7-dichloro-10-iodo-3,4-dihydro-1H-pyrazino[1,2-a]indole-2-carboxylate (1.30 g, 1.79 mmol), 1-(tetrahydro-2H-pyran-2-yl)-4-(4,4,5,5-tetramethyl-1,3,2-dioxaborolan-2-yl)-1H-pyrazole (780 mg, 2.66 mmol), sodium carbonate (380 mg, 3.59 mmol) and Pd(dppf)Cl₂ (130 mg, 0.174 mmol) in 1,4-dioxane (40 mL) and water (4 mL). Purge with N₂ and stir at 90° C. for 2 hrs. Concentrate under vacuum and purify by flash column chromatography eluting with EtOAc in petroleum ether to give tert-butyl 9-(tert-butoxycarbonylamino)-6,7-dichloro-10-(1-tetrahydropyran-2-ylpyrazol-4-yl)-3,4-dihydro-1H-pyrazino[1,2-a]indole-2-carboxylate (600 mg, 50%) as a yellow solid. ES/MS (m/z): (³⁵Cl/³⁷Cl) 606.2/608.2 (M+H).

### Intermediate 361

### 6,7-Dichloro-10-(1H-pyrazol-4-yl)-1,2,3,4-tetrahydropyrazino[1,2-a]indol-9-amine hydrochloride

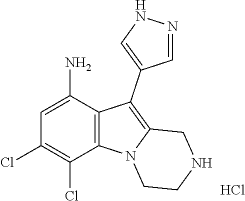 [SMILES: Nc1cc(Cl)c(Cl)c2c1c(-c1cn[nH]c1)c1n2CCNC1 ; Cl]

Dissolve tert-butyl 9-(tert-butoxycarbonylamino)-6,7-dichloro-10-(1-tetrahydropyran-2-ylpyrazol-4-yl)-3,4-dihydro-1H-pyrazino[1,2-a]indole-2-carboxylate (100 mg, 0.165 mmol) in 4M HCl/MeOH (4 mL) and stir at ambient temperature for 2 hrs. Concentrate under vacuum to give 6,7-dichloro-10-(1H-pyrazol-4-yl)-1,2,3,4-tetrahydropyrazino[1,2-a]indol-9-amine hydrochloride (60 mg, 88%) as a yellow solid. ES/MS (m/z): (³⁵Cl/³⁷Cl) 322.0/324.0 (M+H).

### Intermediate 362

### 1-(9-Amino-6,7-dichloro-10-(1H-pyrazol-4-yl)-3,4-dihydropyrazino[1,2-a]indol-2(1H)-yl)-2-hydroxyethan-1-one

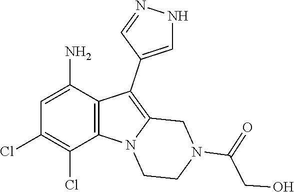 [SMILES: Nc1cc(Cl)c(Cl)c2c1c(-c1cn[nH]c1)c1n2CCN(C(=O)CO)C1]

Dissolve 6,7-dichloro-10-(1H-pyrazol-4-yl)-1,2,3,4-tetrahydropyrazino[1,2-a]indol-9-amine hydrochloride (60 mg, 0.15 mmol), glycolic acid (43 mg, 0.030 mmol) and DIPEA (0.25 mL, 1.43 mmol) in DMF (5 mL). Add HATU (181 mg, 0.467 mmol) and stir at ambient temperature for 1 hr. Dilute with saturated aqueous Na₂CO₃ and extract with DCM/MeOH (10/1), wash with saturated aqueous NaCl, dry over anhydrous Na₂SO₄, filter, and concentrate under vacuum to give 1-(9-amino-6,7-dichloro-10-(1H-pyrazol-4-yl)-3,4-dihydropyrazino[1,2-a]indol-2(1H)-yl)-2-hydroxyethan-1-one (70 mg, 97%) as a yellow solid. ES/MS (m/z): (³⁵Cl/³⁷Cl) 380.0/381.8 (M+H).

### EXAMPLE 145

### N-(6,7-Dichloro-2-(2-hydroxyacetyl)-10-(1H-pyrazol-4-yl)-1,2,3,4-tetrahydropyrazino[1,2-a]indol-9-yl)-2-hydroxyacetamide

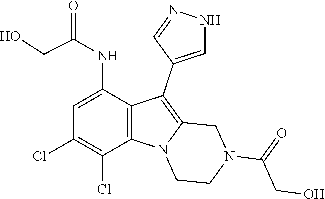 [SMILES: O=C(CO)Nc1cc(Cl)c(Cl)c2c1c(-c1cn[nH]c1)c1n2CCN(C(=O)CO)C1]

Dissolve 1-(9-amino-6,7-dichloro-10-(1H-pyrazol-4-yl)-3,4-dihydropyrazino[1,2-a]indol-2(1H)-yl)-2-hydroxyethan-1-one (100 mg, 0.184 mmol) and TEA (0.040 mL, 0.29 mmol) in DCM (5 mL). Add acetoxyacetyl chloride (0.030 mL, 0.28 mmol) and stir at 0° C. for 1 hr. Dilute with water, extract with DCM, wash with saturated aqueous NaCl, dry over sodium sulfate, filter, and concentrate under vacuum. Dissolve the residue in THF (10 mL) and water (2 mL), Add LiOH (50 mg, 2.09 mmol) and stir at ambient temperature for 0.5 hr. Concentrate under vacuum and purify by prep-HPLC to give N-(6,7-dichloro-2-(2-hydroxyacetyl)-10-(1H-pyrazol-4-yl)-1,2,3,4-tetrahydropyrazino[1,2-a]indol-9-yl)-2-hydroxyacetamide (7.3 mg, 9%) as a white solid. ES/MS (m/z): (³⁵Cl/³⁷Cl) 438.3/440.3 (M+H).

### Intermediate 363

### tert-Butyl 9-amino-6,7-dichloro-10-(1H-pyrazol-4-yl)-3,4-dihydro-1H-pyrazino[1,2-a]indole-2-carboxylate

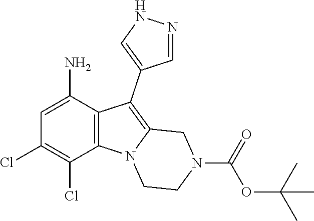 [SMILES: CC(C)(C)OC(=O)N1CCn2c(c(-c3cn[nH]c3)c3c(N)cc(Cl)c(Cl)c32)C1]

Dissolve 6,7-dichloro-10-(1H-pyrazol-4-yl)-1,2,3,4-tetrahydropyrazino[1,2-a]indol-9-amine (300 mg, 0.837 mmol) and TEA (0.26 mL, 1.87 mmol) in DCM (10 mL). Add Boc₂O (0.23 mL, 1.04 mmol) and stir at ambient temperature for 2 hrs. Concentrate to give tert-butyl 9-amino-6,7-dichloro-10-(1H-pyrazol-4-yl)-3,4-dihydro-1H-pyrazino[1,2-a]indole-2-carboxylate (350 mg, 99%) as a yellow solid. ES/MS (m/z): (³⁵Cl/³⁷Cl) 422.0/424.0 (M+H).

### Intermediate 364

### tert-Butyl 6, 7-dichloro-9-[(2-hydroxy acetyl)amino]-10-(1H-pyrazol-4-yl)-3,4-dihydro-1H-pyrazino[1,2-a]indole-2-carboxylate

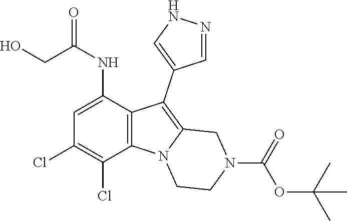 [SMILES: CC(C)(C)OC(=O)N1CCn2c(c(-c3cn[nH]c3)c3c(NC(=O)CO)cc(Cl)c(Cl)c32)C1]

Dissolve tert-butyl 9-amino-6,7-dichloro-10-(1H-pyrazol-4-yl)-3,4-dihydro-1H-pyrazino[1,2-a]indole-2-carboxylate (350 mg, 0.829 mmol) and TEA (0.25 mL, 1.79 mmol) in DCM (10 mL). Add acetoxyacetyl chloride (0.17 mL, 1.58 mmol) and stir at ambient temperature for 2 hrs. Dilute with water and extract with DCM (2×). Wash the combined organic layers with saturated aqueous NaCl, dry over anhydrous sodium sulfate, filter, and concentrate under vacuum. Dissolve the residue in THF (10 mL) and water (5 mL) and add LiOH (200 mg, 8.35 mmol). Stir at ambient temperature for 2 hrs. Dilute with water and extract with DCM (2×). Wash the combined organic layers with saturated aqueous NaCl, dry over anhydrous sodium sulfate, filter, and concentrate under vacuum to give tert-butyl 6,7-dichloro-9-[(2-hydroxyacetyl)amino]-10-(1H-pyrazol-4-yl)-3,4-dihydro-1H-pyrazino[1,2-a]indole-2-carboxylate (400 mg, 90%) as a yellow solid.

### Intermediate 365

### N-(6,7-Dichloro-10-(1H-pyrazol-4-yl)-1,2,3,4-tetrahydropyrazino[1,2-a]indol-9-yl)-2-hydroxyacetamide hydrochloride

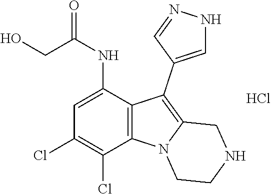 [SMILES: O=C(CO)Nc1cc(Cl)c(Cl)c2c1c(-c1cn[nH]c1)c1n2CCNC1 ; Cl]

Dissolve tert-butyl 6,7-dichloro-9-[(2-hydroxyacetyl)amino]-10-(1H-pyrazol-4-yl)-3,4-dihydro-1H-pyrazino[1,2-a]indole-2-carboxylate (400 mg, 0.750 mmol) in 4M HCl/dioxane (10 mL) and stir at ambient temperature for 2 hrs. Concentrate under vacuum to give N-(6,7-dichloro-10-(1H-pyrazol-4-yl)-1,2,3,4-tetrahydropyrazino[1,2-a]indol-9-yl)-2-hydroxyacetamide hydrochloride (300 mg, 96%) as a yellow solid. ES/MS (m/z): (³⁵Cl/³⁷Cl) 380.0/382.0 (M+H).

### EXAMPLE 146

### N-(2-Acetyl-6,7-dichloro-10-(1H-pyrazol-4-yl)-1,2,3,4-tetrahydropyrazino[1,2-a]indol-9-yl)-2-hydroxyacetamide

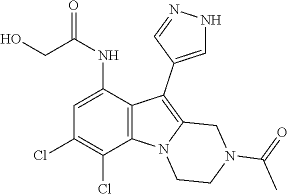 [SMILES: CC(=O)N1CCn2c(c(-c3cn[nH]c3)c3c(NC(=O)CO)cc(Cl)c(Cl)c32)C1]

Dissolve N-(6,7-dichloro-10-(1H-pyrazol-4-yl)-1,2,3,4-tetrahydropyrazino[1,2-a]indol-9-yl)-2-hydroxyacetamide hydrochloride (170 mg, 0.447 mmol) and TEA (0.187 mL, 1.34 mmol) in DCM (5 mL). Add acetyl chloride (0.040 mL, 0.56 mmol) at 0° C. and stir at ambient temperature for 2 hrs. Quench with water, extract with DCM (2×), wash the combined organic layers with saturated aqueous NaCl, dry over sodium sulfate, filter, and concentrate under vacuum. Dissolve the residue in THF (5 mL) and water (5 mL) and add LiOH (110 mg, 4.59 mmol), then stir at ambient temperature for 1 hr. Concentrate under vacuum and purify by reverse phase prep-HPLC to give N-(2-acetyl-6,7-dichloro-10-(1H-pyrazol-4-yl)-1,2,3,4-tetrahydropyrazino[1,2-a]indol-9-yl)-2-hydroxyacetamide (41.95 mg, 22%) as a white solid. ES/MS (m/z): (³⁵Cl/³⁷Cl) 422.3/424.3 (M+H).

### Intermediate 366

### tert-Butyl (4-(6,7-dichloro-10-(1H-pyrazol-4-yl)-1,2,3,4-tetrahydropyrazino[1,2-a]indole-2-carbonyl)tetrahydro-2H-pyran-4-yl)carbamate

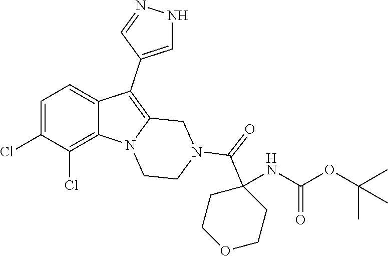 [SMILES: CC(C)(C)OC(=O)NC1(C(=O)N2CCn3c(c(-c4cn[nH]c4)c4ccc(Cl)c(Cl)c43)C2)CCOCC1]

Dissolve 4-N-boc-amino-4-carboxytetrahydropyran (250 mg, 0.968 mmol), HATU (400 mg, 1.02 mmol) and DIPEA (0.6 mL, 3 mmol) in DMF (10 mL), and stir at ambient temperature for 15 min. Add 6,7-dichloro-10-(1H-pyrazol-4-yl)-1,2,3,4-tetrahydropyrazino[1,2-a]indole (200 mg, 0.651 mmol) and stir at ambient temperature for 3 hrs., under N₂ atmosphere. Dilute with water and extract with EtOAc. Wash the organic layer with water (5×). Extract the combined aqueous layers with EtOAc (2×). Wash the combined organic layers with saturated aqueous NaCl, dry over anhydrous Na₂SO₄, filter, and concentrate under vacuum. Purify by flash silica gel chromatography eluting with MeOH in DCM to give tert-butyl (4-(6,7-dichloro-10-(1H-pyrazol-4-yl)-1,2,3,4-tetrahydropyrazino[1,2-a]indole-2-carbonyl)tetrahydro-2H-pyran-4-yl)carbamate (220 mg, 57%) as a yellow solid. ES/MS (m/z): (³⁵Cl/³⁷Cl) 532.3/534.2 (M+H) and 478.1/480.2 (M+H-tBu).

### EXAMPLE 147

### (4-Aminotetrahydro-2H-pyran-4-yl)(6,7-dichloro-10-(1H-pyrazol-4-yl)-3,4-dihydropyrazino[1,2-a]indol-2(1H)-yl)methanone

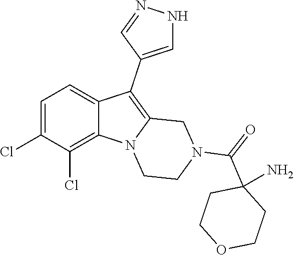 [SMILES: NC1(C(=O)N2CCn3c(c(-c4cn[nH]c4)c4ccc(Cl)c(Cl)c43)C2)CCOCC1]

Dissolve tert-butyl (4-(6,7-dichloro-10-(1H-pyrazol-4-yl)-1,2,3,4-tetrahydropyrazino[1,2-a]indole-2-carbonyl)tetrahydro-2H-pyran-4-yl)carbamate (100 mg, 0.168 mmol) in 4M HCl in MeOH (3 mL) and stir at ambient temperature for 2.5 hrs. Concentrate under vacuum and purify by prep-HPLC to give (4-aminotetrahydro-2H-pyran-4-yl)(6,7-dichloro-10-(1H-pyrazol-4-yl)-3,4-dihydropyrazino[1,2-a]indol-2(1H)-yl)methanone (44.4 mg, 60%) as a white solid. ES/MS (m/z): (³⁵Cl/³⁷Cl) 434.3/436.3 (M+H).

### Intermediate 367

### tert-butyl 9-[tert-butoxycarbonyl(ethyl)amino]-6,7-dichloro-10-(1-tetrahydropyran-2-ylpyrazol-4-yl)-3,4-dihydro-1H-pyrazino[1,2-a]indole-2-carboxylate

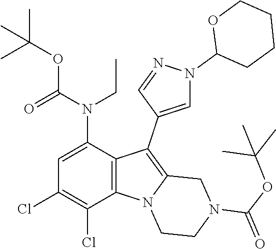 [SMILES: CCN(C(=O)OC(C)(C)C)c1cc(Cl)c(Cl)c2c1c(-c1cnn(C3CCCCO3)c1)c1n2CCN(C(=O)OC(C)(C)C)C1]

Dissolve tert-butyl 9-(tert-butoxycarbonylamino)-6,7-dichloro-10-(1-tetrahydropyran-2-ylpyrazol -4-yl)-3,4-dihydro-1H-pyrazino[1,2-a]indole-2-carboxylate (100 mg, 0.165 mmol) in DMF (5 mL) and add sodium hydride (15 mg, 0.38 mmol, 60% dispersion in mineral oil). Add iodoethane (0.030 mL, 0.37 mmol) and stir at ambient temperature for 2 hrs., under N₂ atmosphere. Concentrate under vacuum and purify by flash column chromatography eluting with EtOAc in petroleum ether to give tert-butyl 9-[tert-butoxycarbonyl(ethyl)amino]-6, 7-dichloro-10-(1-tetrahydropyran-2-ylpyrazol-4-yl)-3,4-dihydro-1H-pyrazino[1,2-a]indole-2-carboxylate (71 mg, 68%) as a yellow solid.

### Intermediate 368

### 6,7-Dichloro-N-ethyl-10-(1H-pyrazol-4-yl)-1,2,3,4-tetrahydropyrazino[1,2-a]indol-9-amine

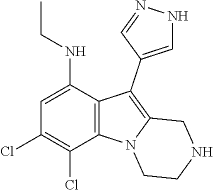 [SMILES: CCNc1cc(Cl)c(Cl)c2c1c(-c1cn[nH]c1)c1n2CCNC1]

Dissolve tert-butyl 9-[tert-butoxycarbonyl(ethyl)amino]-6,7-dichloro-10-(1-tetrahydropyran-2-ylpyrazol-4-yl)-3,4-dihydro-1H-pyrazino[1,2-a]indole-2-carboxylate (71 mg, 0.11 mmol) in 4M HCl in MeOH (4 mL) and stir at ambient temperature for 2 hrs. Concentrate under vacuum to give 6,7-dichloro-N-ethyl-10-(1H-pyrazol-4-yl)-1,2,3,4-tetrahydropyrazino[1,2-a]indol-9-amine (48 mg, crude, HCl salt) as a yellow solid. ES/MS (m/z): (³⁵Cl/³⁷Cl) 349.9/352.0 (M+H).

### EXAMPLE 148

### 1-(6,7-Dichloro-9-(ethylamino)-10-(1H-pyrazol-4-yl)-3,4-dihydropyrazino[1,2-a]indol-2(1H)-yl)-2-hydroxyethan-1-one

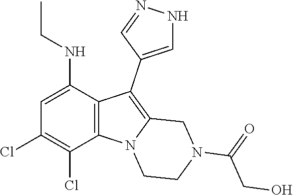 [SMILES: CCNc1cc(Cl)c(Cl)c2c1c(-c1cn[nH]c1)c1n2CCN(C(=O)CO)C1]

Dissolve 6,7-dichloro-N-ethyl-10-(1H-pyrazol-4-yl)-1,2,3,4-tetrahydropyrazino[1,2-a]indol-9-amine (48 mg, 0.12 mmol, HCl salt), glycolic acid (30 mg, 0.39 mmol), DIPEA (160 mg, 1.24 mmol) and HATU (120 mg, 0.309 mmol) in DMF (5 mL). Stir at ambient temperature for 16 hrs under N₂ atmosphere. Concentrate under vacuum and purify by prep-HPLC to give 1-(6,7-dichloro-9-(ethylamino)-10-(1H-pyrazol-4-yl)-3,4-dihydropyrazino[1,2-a]indol-2(1H)-yl)-2-hydroxyethan-1-one (18.30 mg, 35%) as a yellow solid. ES/MS (m/z): (³⁵Cl/³⁷Cl) 408.2/410.2 (M+H).

### Intermediate 369

### tert-Butyl N-(6, 7-dichloro-2-methyl-1-oxo-3,4-dihydropyrazino[1,2-a]indol-9-yl)-N-ethyl-carbamate

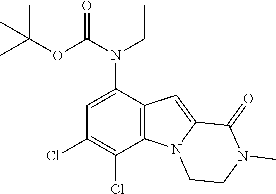 [SMILES: CCN(C(=O)OC(C)(C)C)c1cc(Cl)c(Cl)c2c1cc1n2CCN(C)C1=O]

Dissolve tert-butyl N-(6,7-dichloro-2-methyl-1-oxo-3,4-dihydropyrazino[1,2-a]indol-9-yl)carbamate (240 mg, 0.593 mmol) in DMF (5 mL). Add sodium hydride (65 mg, 1.6 mmol, 60% dispersion in mineral oil) at 0° C. and stir for 0.5 hrs. Add iodoethane (0.075 mL, 0.94 mmol) at 0° C. and stir at ambient temperature for 2 hrs. Dilute with water and extract with EtOAc (3×). Wash the combined organic layers with 4% aqueous LiCl (3×), dry over anhydrous sodium sulfate, filter, and concentrate under vacuum to give tert-butyl N-(6,7-dichloro-2-methyl-1-oxo-3,4-dihydropyrazino[1,2-a]indol-9-yl)-N-ethyl-carbamate (300 mg, 98%) as a yellow solid.

### Intermediate 370

### tert-Butyl N-(10-bromo-6,7-dichloro-2-methyl-1-oxo-3,4-dihydropyrazino[1,2-a]indol-9-yl)-N-ethyl-carbamate

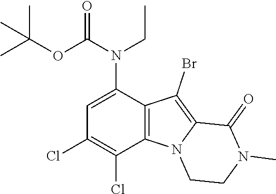 [SMILES: CCN(C(=O)OC(C)(C)C)c1cc(Cl)c(Cl)c2c1c(Br)c1n2CCN(C)C1=O]

Dissolve tert-butyl N-(6,7-dichloro-2-methyl-1-oxo-3,4-dihydropyrazino[1,2-a]indol-9-yl)-N-ethyl-carbamate (300 mg, 0.582 mmol) in DMF (5 mL). Add NBS (170 mg, 0.955 mmol) and stir at ambient temperature for 2 hrs. Dilute with water and extract with EtOAc (3×). Wash the combined organic layers with 4% aqueous LiCl (3×), dry over anhydrous sodium sulfate, filter, and concentrate under vacuum to give tert-butyl N-(10-bromo-6,7-dichloro-2-methyl-1-oxo-3,4-dihydropyrazino[1,2-a]indol-9-yl)-N-ethyl-carbamate (300 mg, 89%) as a yellow gum.

### Intermediate 371

### tert-Butyl N-[6,7-dichloro-2-methyl-1-oxo-10-(1-tetrahydropyran-2-ylpyrazol-4-yl)-3,4-dihydropyrazino[1,2-a]indol-9-yl]-N-ethyl-carbamate

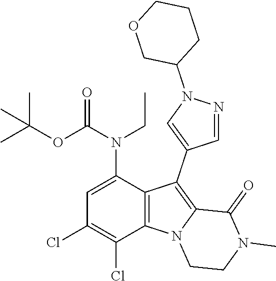 [SMILES: CCN(C(=O)OC(C)(C)C)c1cc(Cl)c(Cl)c2c1c(-c1cnn(C3CCCOC3)c1)c1n2CCN(C)C1=O]

Suspend give tert-butyl N-(10-bromo-6,7-dichloro-2-methyl-1-oxo-3,4-dihydropyrazino[1,2-a]indol-9-yl)-N-ethyl-carbamate (300 mg, 0.550 mmol), 1-(tetrahydro-2H-pyran-2-yl)-4-(4,4,5,5-tetramethyl-1,3,2-dioxaborolan-2-yl)-1H-pyrazole (322 mg, 1.10 mmol), K₂CO₃ (110 mg, 1.11 mmol) and Pd(dppf)Cl₂ (45 mg, 0.058 mmol) in 1,4-dioxane (8 mL) and water (2 mL). Purge with N₂ (3×) and heat at 90° C. for 2 hrs. Cool to ambient temperature, dilute with water, and extract with EtOAc (3×). Dry the combined organic layers over anhydrous sodium sulfate, filter, and concentrate under vacuum. Purify by flash column chromatography eluting with MeOH in DCM to give tert-butyl N-[6,7-dichloro-2-methyl-1-oxo-10-(1-tetrahydropyran-2-ylpyrazol-4-yl)-3,4-dihydropyrazino[1,2-a]indol-9-yl]-N-ethyl-carbamate (230 mg, 59%) as a yellow gum. ES/MS (m/z): (³⁵Cl/³⁷Cl) 562.2/564.2 (M+H).

### EXAMPLE 149

### 6,7-Dichloro-9-(ethyl amino)-2-methyl-10-(1H-pyrazol-4-yl)-3,4-dihydropyrazino[1,2-a]indol-1-one hydrochloride

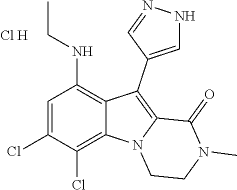

Suspend tert-butyl N-[6,7-dichloro-2-methyl-1-oxo-10-(1-tetrahydropyran-2-ylpyrazol-4-yl)-3,4-dihydropyrazino[1,2-a]indol-9-yl]-N-ethyl-carbamate (230 mg, 0.327 mmol) in MeOH (2 mL). Add 4M HCl in MeOH (5 mL) and stir at ambient temperature for 2 hrs. Concentrate under vacuum and purify by prep-HPLC to give 6,7-dichloro-9-(ethylamino)-2-methyl-10-(1H-pyrazol-4-yl)-3,4-dihydropyrazino[1,2-a]indol-1-one hydrochloride (114 mg, 84%, HCl salt) as an off-white solid. ES/MS (m/z): (³⁵Cl/³⁷Cl) 378.2/380.2 (M+H).

### Intermediate 372

### 6,7-Dichloro-N-ethyl-3-(1-tetrahydropyran-2-ylpyrazol-4-yl)-1H-indol-4-amine

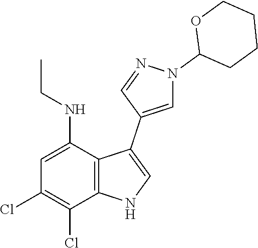 [SMILES: CCNc1cc(Cl)c(Cl)c2[nH]cc(-c3cnn(C4CCCCO4)c3)c12]

Dissolve 4-bromo-6,7-dichloro-1-(p-tolylsulfonyl)-3-(1-tetrahydropyran-2-ylpyrazol-4-yl)indole (200 mg, 0.316 mmol), ethylamine (0.24 mL, 0.48 mmol, 2.0M in THF), Pd(t-Bu₃P)₂ (25 mg, 0.048 mmol) and sodium tert-butoxide (94 mg, 0.95 mmol) in toluene (3 mL). Purge with N₂ and stir at 100° C. in a sealed tube for 16-18 hrs. Combine with another batch run at 0.50×scale for work up and purification. Concentrate under vacuum, dilute with water, extract with EtOAc, dry over anhydrous sodium sulfate, filter, and concentrate under vacuum. Purify by flash column chromatography eluting with EtOAc in petroleum ether to give 6,7-dichloro-N-ethyl-3-(1-tetrahydropyran-2-ylpyrazol-4-yl)-1H-indol-4-amine (110 mg, 24%) as a yellow solid. ES/MS (m/z): (³⁵Cl/³⁷Cl) 379.0/381.1 (M+H).

### EXAMPLE 150

### 6,7-Dichloro-N-ethyl-3-(1H-pyrazol-4-yl)-1H-indol-4-amine

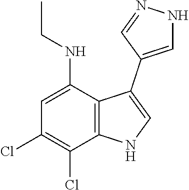 [SMILES: CCNc1cc(Cl)c(Cl)c2[nH]cc(-c3cn[nH]c3)c12]

Dissolve 6,7-dichloro-N-ethyl-3-(1-tetrahydropyran-2-ylpyrazol-4-yl)-1H-indol-4-amine (170 mg, 0.448 mmol) in 4M HCl in MeOH (4 mL) and stir at ambient temperature for 1 hr. Concentrate under vacuum and purify by prep-HPLC and SFC to give 6,7-dichloro-N-ethyl-3-(1H-pyrazol-4-yl)-1H-indol-4-amine (17.8 mg, 13%) as a white solid. ES/MS (m/z): (³⁵Cl/³⁷Cl) 295.2/297.2 (M+H).

### Intermediate 373

### tert-Butyl 2-[2-[6,7-dichloro-9-(cyanomethoxy)-10-(1H-pyrazol-4-yl)-3,4-dihydro-1H-pyrazino[1,2-a]indol-2-yl]ethyl]morpholine-4-carboxylate

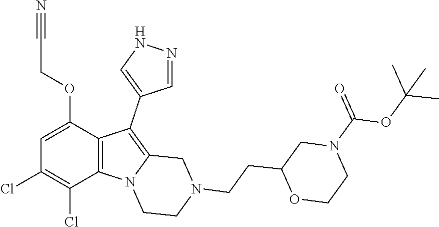 [SMILES: CC(C)(C)OC(=O)N1CCOC(CCN2CCn3c(c(-c4cn[nH]c4)c4c(OCC#N)cc(Cl)c(Cl)c43)C2)C1]

Dissolve 2-((6,7-dichloro-10-(1H-pyrazol-4-yl)-1,2,3,4-tetrahydropyrazino[1,2-a]indol-9-yl)oxy)acetonitrile (480 mg, 1.20 mmol, HCl salt) in MeOH (15 mL). Add DIPEA (240 mg, 1.86 mmol) and stir at ambient temperature for 0.5 hr. Add acetic acid (150 mg, 2.49 mmol) and stir for another 0.5 hr. Then add tert-butyl 2-(2-oxoethyl)morpholine-4-carboxylate (900 mg, 3.53 mmol) and stir at ambient temperature for 0.5 hr. Add sodium cyanoborohydride (240 mg, 3.63 mmol) and stir at ambient temperature for 16-18 hrs. Quench with saturated aqueous NaHCO₃ and extract with EtOAc (3×). Dry the combined organic layers over anhydrous sodium sulfate, filter, and concentrate under vacuum. Purify by flash column chromatography eluting with MeOH in DCM to give tert-butyl 2-[2-[6,7-dichloro-9-(cyanomethoxy)-10-(1H-pyrazol-4-yl)-3,4-dihydro-1H-pyrazino[1,2-a]indol-2-yl]ethyl]morpholine-4-carboxylate (600 mg, 88%) as a light-yellow solid. ES/MS (m/z): (³⁵Cl/³⁷Cl) 575.1/577.0 (M+H).

### Intermediate 374

### tert-Butyl 2-[2-[6,7-dichloro-9-(cyanomethoxy)-1-oxo-10-(1H-pyrazol-4-yl)-3,4-dihydropyrazino[1,2-a]indol-2-yl]ethyl]morpholine-4-carboxylate

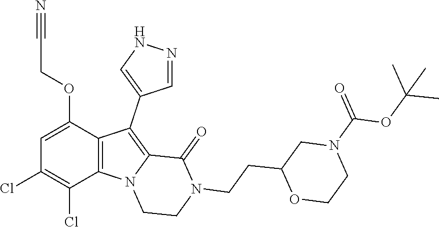 [SMILES: CC(C)(C)OC(=O)N1CCOC(CCN2CCn3c(c(-c4cn[nH]c4)c4c(OCC#N)cc(Cl)c(Cl)c43)C2=O)C1]

Dissolve tert-butyl 2-[2-[6,7-dichloro-9-(cyanomethoxy)-10-(1H-pyrazol-4-yl)-3,4-dihydro-1H-pyrazino[1,2-a]indol-2-yl]ethyl]morpholine-4-carboxylate (200 mg, 0.313 mmol) and sodium bicarbonate (300 mg, 3.55 mmol) in THF (7 mL) and water (3 mL). Add iodine (603 mg, 2.38 mmol) and stir at ambient temperature for 4 hrs. Combine with two other batches run at the same scale for work up and purification. Quench with saturated aqueous sodium thiosulfate solution and saturated aqueous NaHCO₃ solution, extract with EtOAc, wash with saturated aqueous NaHCO₃ solution, dry over anhydrous sodium sulfate, filter, and concentrate under vacuum. Purify by flash column chromatography eluting with MeOH in DCM to give tert-butyl 2-[2-[6,7-dichloro-9-(cyanomethoxy)-1-oxo-10-(1H-pyrazol-4-yl)-3,4-dihydropyrazino[1,2-a]indol-2-yl]ethyl]morpholine-4-carboxylate (180 mg, 27%) as a yellow gum. ES/MS (m/z): (³⁵Cl/³⁷Cl) 589.1/591.1 (M+H) and 489.0/490.9 (M+H-Boc).

### Intermediate 375

### 2-[[6,7-Dichloro-2-(2-morpholin-2-yl ethyl)-1-oxo-10-(1H-pyrazol-4-yl)-3,4-dihydropyrazino[1,2-a]indol-9-yl]oxy]acetonitrile

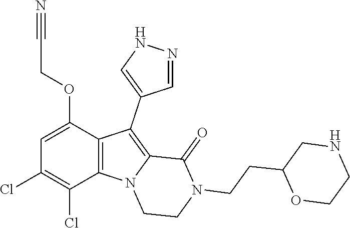 [SMILES: N#CCOc1cc(Cl)c(Cl)c2c1c(-c1cn[nH]c1)c1n2CCN(CCC2CNCCO2)C1=O]

Suspend tert-butyl 2-[2-[6,7-dichloro-9-(cyanomethoxy)-1-oxo-10-(1H-pyrazol-4-yl)-3,4-dihydropyrazino[1,2-a]indol-2-yl]ethyl]morpholine-4-carboxylate (180 mg, 0.253 mmol) and anisole (300 mg, 2.77 mmol) in DCM (2 mL). Add TFA (1 mL) and stir at ambient temperature for 0.5 hr. Concentrate under vacuum, quench with saturated aqueous sodium carbonate, extract with EtOAc, dry over anhydrous sodium sulfate, filter, and concentrate under vacuum to give 2-[[6,7-dichloro-2-(2-morpholin-2-ylethyl)-1-oxo-10-(1H-pyrazol-4-yl)-3,4-dihydropyrazino[1,2-a]indol-9-yl]oxy]acetonitrile (150 mg, 86%) as a yellow gum. ES/MS (m/z): (³⁵Cl/³⁷Cl) 489.0/490.9 (M+H).

### EXAMPLE 151

### 2-[[2-[2-(4-Acetylmorpholin-2-yl)ethyl]-6,7-dichloro-1-oxo-10-(1H-pyrazol-4-yl)-3,4-dihydropyrazino[1,2-a]indol-9-yl]oxy]acetonitrile

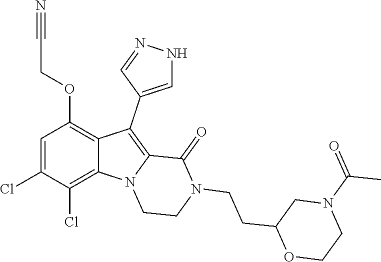 [SMILES: CC(=O)N1CCOC(CCN2CCn3c(c(-c4cn[nH]c4)c4c(OCC#N)cc(Cl)c(Cl)c43)C2=O)C1]

Suspend 2-[[6,7-dichloro-2-(2-morpholin-2-ylethyl)-1-oxo-10-(1H-pyrazol-4-yl)-3,4-dihydropyrazino[1,2-a]indol-9-yl]oxy]acetonitrile (150 mg, 0.187 mmol) and TEA (60 mg, 0.59 mmol) in DCM (6 mL). Add acetic anhydride (20 mg, 0.20 mmol) at 0° C. Stir at 0° C. for 2 hrs. Concentrate under vacuum, quench with saturated aqueous sodium bicarbonate, and purify by prep-HPLC to give 2-[[2-[2-(4-acetylmorpholin-2-yl)ethyl]-6,7-dichloro-1-oxo-10-(1H-pyrazol-4-yl)-3,4-dihydropyrazino[1,2-a]indol-9-yl]oxy]acetonitrile (25.7 mg, 26%) as a white solid. ES/MS (m/z): (³⁵Cl/³⁷Cl) 531.4/533.3 (M+H).

### Intermediate 376

### 2-(6-Chloro-5-methoxy-indol-1-yl)ethynyl-triisopropyl-silane

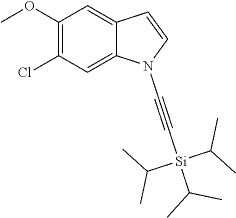 [SMILES: COc1cc2ccn(C#C[Si](C(C)C)(C(C)C)C(C)C)c2cc1Cl]

Dissolve 6-chloro-5-methoxy-1H-indole (1.00 g, 5.34 mmol) in 1,4-dioxane (20 mL) and add (bromoethynyl)triisopropylsilane (7.19 g, 26.7 mmol), PEG-400 (200 mg), CuI (102 mg, 0.536 mmol) and Cs₂CO₃ (2.09 g, 6.41 mmol). Purge with N₂. Stir at 135° C. under N₂ for 16 hrs. Dilute with water and extract with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give 2-(6-chloro-5-methoxy-indol-1-yl)ethynyl-triisopropyl-silane (1.50 g, 70%) as a yellow oil.

### Intermediate 377

### 6-Chloro-1-ethynyl-5-methoxy-indole

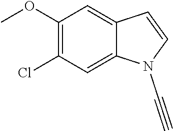

Dissolve 2-(6-chloro-5-methoxy-indol-1-yl)ethynyl-triisopropyl-silane (1.45 g, 3.60 mmol) in THF (10 mL) and add TBAF (11 mL, 1M in THF). Stir at ambient temperature for 2 hrs. Dilute with EtOAc, wash with saturated aqueous NH₄Cl, dry over anhydrous Na₂SO₄, filter, and concentrate under vacuum. Purify by flash column chromatography eluting with EtOAc in petroleum ether to give 6-chloro-1-ethynyl-5-methoxy-indole (955 mg, 94%) as a yellow solid. ES/MS (m/z): (³⁵Cl/³⁷Cl) 411.1/413.0 (2M+H).

### Intermediate 378

### tert-Butyl-[2-[4-(6-chloro-5-methoxy-indol-1-yl)triazol-1-yl]ethoxy]-dimethyl-silane

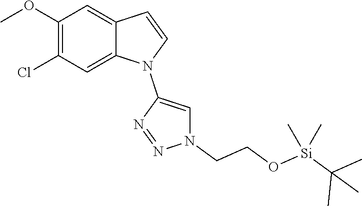 [SMILES: COc1cc2ccn(-c3cn(CCO[Si](C)(C)C(C)(C)C)nn3)c2cc1Cl]

Dissolve 6-chloro-1-ethynyl-5-methoxy-indole (855 mg, 3.12 mmol) in DMF (8 mL) and H₂O (8 mL). Add copper (40 mg, 0.63 mmol), CuSO₄ (100 mg, 0.627 mmol) and (2-azidoethoxy)(tert-butyl)dimethylsilane (794 mg, 3.75 mmol). Purge with N₂. Stir at 110° C. under N₂ for 0.5 hr. Combine with another batch run at 0.12×scale for work up and purification. Dilute with water and extract with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give tert-butyl-[2-[4-(6-chloro-5-methoxy-indol-1-yl)triazol-1-yl]ethoxy]-dimethyl-silane (605 mg, 38%) as a yellow oil.

### Intermediate 379

### tert-Butyl-[2-[4-(6-chloro-3-iodo-5-methoxy-indol-1-yl)triazol-1-yl]ethoxy]-dimethyl-silane

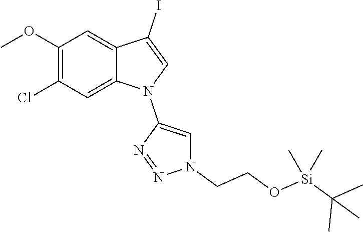 [SMILES: COc1cc2c(I)cn(-c3cn(CCO[Si](C)(C)C(C)(C)C)nn3)c2cc1Cl]

Dissolve tert-butyl-[2-[4-(6-chloro-5-methoxy-indol-1-yl)triazol-1-yl]ethoxy]-dimethyl-silane (585 mg, 1.29 mmol) in DMF (8 mL) and add NIS (357 mg, 1.56 mmol). Stir at ambient temperature for 1.5 hr. Quench with saturated aqueous NaHCO₃, extract with EtOAc, wash with saturated aqueous NaCl, dry over anhydrous Na₂SO₄, filter, and concentrate under vacuum. Purify by flash column chromatography eluting with EtOAc in petroleum ether to give tert-butyl-[2-[4-(6-chloro-3-iodo-5-methoxy-indol-1-yl)triazol -1-yl]ethoxy]-dimethyl-silane (615 mg, 85%) as a yellow oil.

### Intermediate 380

### 1-(1-(2-((tert-Butyl dimethyl silyl)oxy)ethyl)-1H-1,2,3-triazol-4-yl)-6-chloro-5-methoxy -3-(1-(tetrahydro-2H-pyran-2-yl)-1H-pyrazol-4-yl)-1H-indole

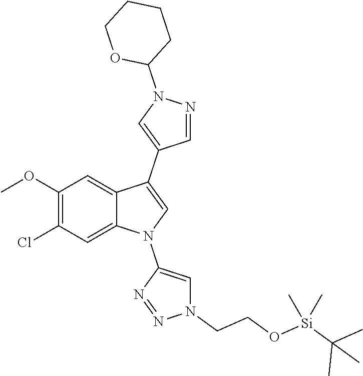 [SMILES: COc1cc2c(-c3cnn(C4CCCCO4)c3)cn(-c3cn(CCO[Si](C)(C)C(C)(C)C)nn3)c2cc1Cl]

Dissolve tert-butyl-[2-[4-(6-chloro-3-iodo-5-methoxy-indol-1-yl)triazol-1-yl]ethoxy]-dimethyl -silane (565 mg, 1.01 mmol) in 1,4-dioxane (4 mL) and H₂O (1 mL). Add Na₂CO₃ (324 mg, 3.03 mmol), Pd(dtbpf)Cl₂ (100 mg, 0.150 mmol) and 1-(tetrahydro-2H-pyran-2-yl)-4-(4,4,5,5-tetramethyl-1,3,2-dioxaborolan-2-yl)-1H-pyrazole (442 mg, 1.56 mmol). Purge with N₂. Stir at 90° C. under N₂ for 2 hrs. Dilute with EtOAc, wash with saturated aqueous NaCl, dry over anhydrous Na₂SO₄, filter, and concentrate under vacuum. Purify by flash column chromatography eluting with EtOAc in petroleum ether to give 1-(1-(2-((tert-butyldimethyl silyl)oxy)ethyl)-1H-1,2,3-triazol-4-yl)-6-chloro-5-methoxy-3-(1-(tetrahydro-2H-pyran-2-yl)-1H-pyrazol-4-yl)-1H-indole (515 mg, 87%) as a yellow solid. ES/MS (m/z): (³⁵Cl/³⁷Cl) 557.3/559.3 (M+H) and 473.2/475.2 (M+H-THP).

### EXAMPLE 152

### 2-[4-[6-Chloro-5-methoxy-3-(1H-pyrazol-4-yl)indol-1-yl]triazol-1-yl]ethanol

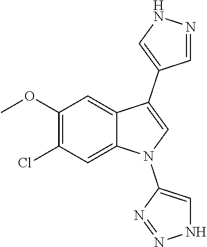 [SMILES: COc1cc2c(-c3cn[nH]c3)cn(-c3c[nH]nn3)c2cc1Cl]

Dissolve 1-(1-(2-((tert-butyldimethylsilyl)oxy)ethyl)-1H-1,2,3-triazol-4-yl)-6-chloro-5-methoxy-3-(1-(tetrahydro-2H-pyran-2-yl)-1H-pyrazol-4-yl)-1H-indole (200 mg, 0.341 mmol) in DCM (3 mL) and add TFA (5 mL). Stir at ambient temperature for 16 hrs. Concentrate and dissolve the residue in THF (4 mL) and H₂O (1 mL). Add LiOH (83 mg, 3.4 mmol). Stir at ambient temperature for 2 hrs. Acidify with 1M aqueous HCl to pH=5 and concentrate under vacuum. Purify by prep-HPLC to give 2-[4-[6-chloro-5-methoxy-3-(1H-pyrazol-4-yl)indol-1-yl]triazol-1-yl]ethanol (49.5 mg, 40%) as a white solid. ES/MS (m/z): (³⁵Cl/³⁷Cl) 359.3/361.3 (M+H).

### Intermediate 381

### 6, 7-Dichloro-2-(tetrahydropyran-2-yloxymethyl)-1H-indol-4-ol

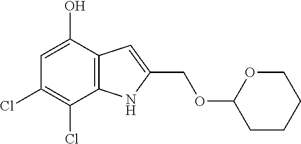 [SMILES: Oc1cc(Cl)c(Cl)c2[nH]c(COC3CCCCO3)cc12]

Dissolve 4-bromo-6,7-dichloro-2-(tetrahydropyran-2-yloxymethyl)-1H-indole (2.07 g, 4.91 mmol) in 1,4-dioxane (20 mL) and water (10 mL). Add t-BuONa (1.22 g, 12.3 mmol) and t-BuBrettphos-Pd-G₃ (220 mg, 0.252 mmol) under N₂ atmosphere. Purge with N₂ then stir at 65° C. for 3 hrs. Quench with saturated aqueous NH₄Cl. Add 1M aqueous HCl to adjust to pH=5-6. Extract with EtOAc (2×), wash the combined organic layers with saturated aqueous NaCl (2×), dry over anhydrous sodium sulfate, filter, and concentrate under vacuum. Purify by flash column chromatography (EtOAc/PE) to give 6,7-dichloro-2-(tetrahydropyran-2-yloxymethyl)-1H-indol-4-ol (1.01 g, 55%) as a yellow oil. ES/MS (m/z): (³⁵Cl/³⁷Cl) 314.0/316.0 (M−H).

### Intermediate 382

### 2-[[6,7-Dichloro-2-(tetrahydropyran-2-yloxymethyl)-1H-indol-4-yl]oxy]acetonitrile

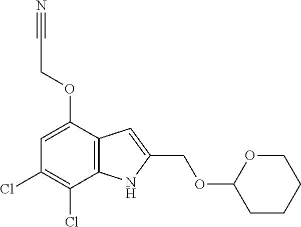 [SMILES: N#CCOc1cc(Cl)c(Cl)c2[nH]c(COC3CCCCO3)cc12]

Dissolve 6,7-dichloro-2-(tetrahydropyran-2-yloxymethyl)-1H-indol-4-ol (1.01 g, 2.72 mmol) and potassium carbonate (420 mg, 3.04 mmol) in DMF (10 mL). Add bromoacetonitrile (0.4 mL, 6.0 mmol) at 0° C. and stir for 3 hrs. Dilute with EtOAc, wash with water, dry over anhydrous sodium sulfate, filter, and concentrate under vacuum. Purify by flash column chromatography eluting with EtOAc in petroleum ether to give 2-[[6,7-dichloro-2-(tetrahydropyran-2-yloxymethyl)-1H-indol-4-yl]oxy]acetonitrile (710 mg, 66%) as a yellow gum. ES/MS (m/z): (³⁵Cl/³⁷Cl) 353.0/355.0 (M−H).

### Intermediate 383

### 2-[[6,7-Dichloro-2-(hydroxymethyl)-1H-indol-4-yl]oxy]acetonitrile BVC-E20532-084-B

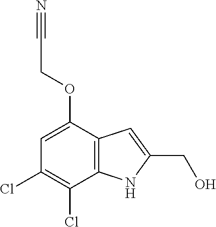 [SMILES: N#CCOc1cc(Cl)c(Cl)c2[nH]c(CO)cc12]

Dissolve 2-[[6, 7-dichloro-2-(tetrahydropyran-2-yloxymethyl)-1H-indol-4-yl]oxy]acetonitrile (550 mg, 1.39 mmol) in THF (5 mL) and add 6M aqueous HCl (5 mL) at ambient temperature. Stir at ambient temperature for 2 hrs. Combine with another batch run at 0.09×scale for work up and purification. Dilute with water, extract with EtOAc, wash with saturated aqueous NaCl, dry over anhydrous sodium sulfate, filter, and concentrate under vacuum. Purify by flash column chromatography eluting with EtOAc in petroleum ether to give 2-[[6,7-dichloro-2-(hydroxymethyl)-1H-indol-4-yl]oxy]acetonitrile (270 mg, 59%) as a yellow solid. ES/MS (m/z): (³⁵Cl/³⁷Cl) 268.6/270.6 (M−H).

### Intermediate 384

### 2-[[6,7-Dichloro-2-(hydroxymethyl)-3-iodo-1H-indol-4-yl]oxy]acetonitrile

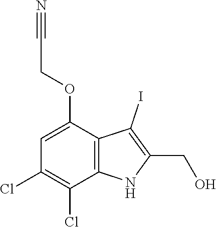 [SMILES: N#CCOc1cc(Cl)c(Cl)c2[nH]c(CO)c(I)c12]

Dissolve 2-[[6,7-dichloro-2-(hydroxymethyl)-1H-indol-4-yl]oxy]acetonitrile (270 mg, 0.896 mmol) in DMF (5 mL) and add NIS (250 mg, 1.08 mmol) at 0° C. Stir at ambient temperature for 1 hr. Quench with saturated aqueous Na₂SO₃ and extract with EtOAc (3×). Wash the combined organic layers with 4% aqueous LiCl (3×), dry over anhydrous sodium sulfate, filter, and concentrate under vacuum to give 2-[[6,7-dichloro-2-(hydroxymethyl)-3-iodo-1H-indol-4-yl]oxy]acetonitrile (327 mg, 87%) as a red solid. ES/MS (m/z): (³⁵Cl/³⁷Cl) 394.6/396.6 (M−H).

### Intermediate 385

### 2-[[6,7-Dichloro-2-(hydroxymethyl)-3-(1-tetrahydropyran-2-ylpyrazol-4-yl)-1H-indol-4-yl]oxy]acetonitrile

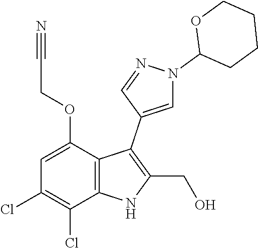 [SMILES: N#CCOc1cc(Cl)c(Cl)c2[nH]c(CO)c(-c3cnn(C4CCCCO4)c3)c12]

Suspend 2-[[6,7-dichloro-2-(hydroxymethyl)-3-iodo-1H-indol-4-yl]oxy]acetonitrile (325 mg, 0.778 mmol), 1-tetrahydropyran-2-yl-4-(4,4,5,5-tetramethyl-1,3,2-dioxaborolan-2-yl)pyrazole (240 mg, 0.863 mmol), sodium carbonate (250 mg, 2.36 mmol) and Pd(dtbpf)Cl₂ (105 mg, 0.158 mmol) in 1,4-dioxane (6 mL) and water (3 mL). Purge with N₂ and stir at 90° C. for 30 min. Dilute with water, extract with EtOAc, wash with saturated aqueous NaCl, dry over anhydrous sodium sulfate, filter, and concentrate under vacuum. Purify by flash column chromatography (MeOH/DCM), then triturate with PE/EtOAc (1/1) to give 2-[[6, 7-dichloro-2-(hydroxymethyl)-3-(1-tetrahydropyran-2-ylpyrazol-4-yl)-1H-indol-4-yl]oxy]acetonitrile (130 mg, 37%) as a yellow solid. ES/MS (m/z): (³⁵Cl/³⁷Cl) 421.0/422.9 (M+H).

### Intermediate 386

### 2-((2-(Azidomethyl)-6,7-dichloro-3-(1-(tetrahydro-2H-pyran-2-yl)-1H-pyrazol-4-yl)-1H-indol-4-yl)oxy)acetonitrile

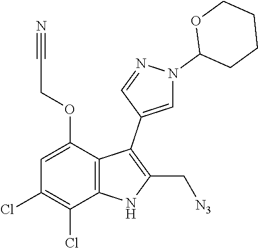 [SMILES: N#CCOc1cc(Cl)c(Cl)c2[nH]c(CN=[N+]=[N-])c(-c3cnn(C4CCCCO4)c3)c12]

Dissolve 2-[[6,7-dichloro-2-(hydroxymethyl)-3-(1-tetrahydropyran-2-ylpyrazol-4-yl)-1H-indol-4-yl]oxy]acetonitrile (111 mg, 0.245 mmol) in THF (2 mL) and add DPPA (110 mg, 0.400 mmol) and DBU (100 mg, 0.657 mmol) at 0° C., then stir at ambient temperature for 16 hrs. Dilute with water, extract with EtOAc, wash with saturated aqueous NaCl, dry over anhydrous sodium sulfate, filter, and concentrate under vacuum. Purify by flash column chromatography eluting with EtOAc in petroleum ether to give 2-((2-(azidomethyl)-6,7-dichloro-3-(1-(tetrahydro-2H-pyran-2-yl)-1H-pyrazol-4-yl)-1H-indol-4-yl)oxy)acetonitrile (70 mg, 61%) as a yellow solid.

### Intermediate 387

### 2-((2-(Aminomethyl)-6, 7-dichloro-3-(1-(tetrahydro-2H-pyran-2-yl)-1H-pyrazol-4-yl)-1H-indol-4-yl)oxy)acetonitrile

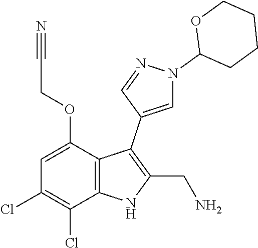 [SMILES: N#CCOc1cc(Cl)c(Cl)c2[nH]c(CN)c(-c3cnn(C4CCCCO4)c3)c12]

Dissolve 2-((2-(azidomethyl)-6,7-dichloro-3-(1-(tetrahydro-2H-pyran-2-yl)-1H-pyrazol-4-yl)-1H-indol-4-yl)oxy)acetonitrile (70 mg, 0.16 mmol) and Ph₃P (50 mg, 0.19 mmol) in THF (1.5 mL) and water (0.1 mL) at 0° C. Stir at ambient temperature for 16 hrs. Concentrate under vacuum to give 2-((2-(aminomethyl)-6,7-dichloro-3-(1-(tetrahydro-2H-pyran-2-yl)-1H-pyrazol-4-yl)-1H-indol-4-yl)oxy)acetonitrile (60 mg, crude) as a yellow solid. ES/MS (m/z): (³⁵Cl/³⁷Cl) 403.0/405.0 (M+H—NH₃).

### Intermediate 388

### N-[[6,7-Dichloro-4-(cyanomethoxy)-3-(1-tetrahydropyran-2-ylpyrazol-4-yl)-1H-indol-2-yl]methyl]acetamide

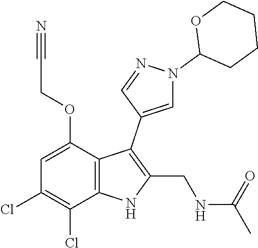 [SMILES: CC(=O)NCc1[nH]c2c(Cl)c(Cl)cc(OCC#N)c2c1-c1cnn(C2CCCCO2)c1]

Dissolve 2-((2-(aminomethyl)-6,7-dichloro-3-(1-(tetrahydro-2H-pyran-2-yl)-1H-pyrazol-4-yl)-1H-indol-4-yl)oxy)acetonitrile (66 mg, 0.16 mmol) and TEA (0.044 mL, 0.32 mmol) in DCM (1 mL). Add acetic anhydride (0.030 mL, 0.32 mmol) at 0° C. and stir at ambient temperature for 16 hrs. Dilute with water, extract with EtOAc, wash with saturated aqueous NaCl, dry over anhydrous sodium sulfate, filter, and concentrate under vacuum to give N-[[6,7-dichloro-4-(cyanomethoxy)-3-(1-tetrahydropyran-2-ylpyrazol-4-yl)-1H-indol-2-yl]methyl]acetamide (60 mg, 83%) as a yellow solid.

### EXAMPLE 153

### N-((6,7-Dichloro-4-(cyanomethoxy)-3-(1H-pyrazol-4-yl)-1H-indol-2-yl)methyl)acetamide

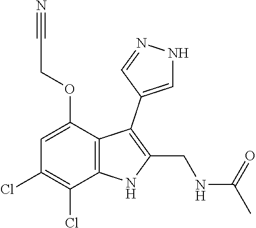 [SMILES: CC(=O)NCc1[nH]c2c(Cl)c(Cl)cc(OCC#N)c2c1-c1cn[nH]c1]

Dissolve N-[[6,7-dichloro-4-(cyanomethoxy)-3-(1-tetrahydropyran-2-ylpyrazol-4-yl)-1H-indol-2-yl]methyl]acetamide (66 mg, 0.14 mmol) and TFA (1 mL) in DCM (1 mL). Stir at ambient temperature for 2 hrs. Concentrate under vacuum and purify by prep-HPLC to give N-((6,7-dichloro-4-(cyanomethoxy)-3-(1H-pyrazol-4-yl)-1H-indol-2-yl)methyl)acetamide (7.83 mg, 15%) as a yellow solid. ES/MS (m/z): (³⁵Cl/³⁷Cl) 378.3/380.2 (M+H).

### Intermediate 389

### tert-Butyl N-(7,8-dichloro-2,3-dihydro-1H-pyrrolo[1,2-a]indol-2-yl)carbamate

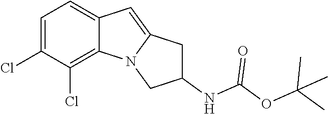 [SMILES: CC(C)(C)OC(=O)NC1Cc2cc3ccc(Cl)c(Cl)c3n2C1]

Dissolve tert-butyl (1-((tert-butyldimethylsilyl)oxy)-3-(6,7-dichloro-1-tosyl-1H-indol-2-yl)propan-2-yl)carbamate (5.0 g, 7.2 mmol) in THF (70 mL). Add TBAF (25 mL, 1.0M in THF). Stir at 80° C. for 16-18 hrs. Cool to ambient temperature and dilute with EtOAc, wash with saturated aqueous NH₄Cl and then with saturated aqueous NaCl, dry over sodium sulfate, filter, and concentrate under vacuum. Purify by flash column chromatography eluting with EtOAc in petroleum ether and then with MeOH in DCM to give tert-butyl N-(7,8-dichloro-2,3-dihydro-1H-pyrrolo[1,2-a]indol-2-yl)carbamate (410 mg, 13%) as a yellow solid. ES/MS (m/z): (³⁵Cl/³⁷Cl) 340.9/342.9 (M+H).

### Intermediate 390

### tert-Butyl (5,6-dichloro-9-iodo-2,3-dihydro-1H-pyrrolo[1,2-a]indol-2-yl)carbamate

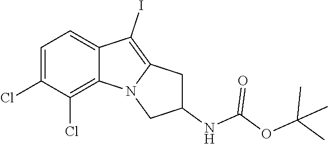 [SMILES: CC(C)(C)OC(=O)NC1Cc2c(I)c3ccc(Cl)c(Cl)c3n2C1]

Dissolve tert-butyl N-(7,8-dichloro-2,3-dihydro-1H-pyrrolo[1,2-a]indol-2-yl)carbamate (310 mg, 0.700 mmol) and NIS (420 mg, 1.83 mmol) in DMF (5 mL). Stir at 25° C. for 1 hr. Combine with another batch run at 0.31×scale for work up and purification. Dilute with water and extract with EtOAc, wash with saturated aqueous NaCl, dry over sodium sulfate, filter, and concentrate under vacuum. Purify by flash column chromatography eluting with EtOAc in petroleum ether to give tert-butyl (5,6-dichloro-9-iodo-2,3-dihydro-1H-pyrrolo[1,2-a]indol-2-yl)carbamate (400 mg, 84%) as a white solid.

### Intermediate 391

### tert-Butyl (5,6-dichloro-9-(1-(tetrahydro-2H-pyran-2-yl)-1H-pyrazol-4-yl)-2,3-dihydro-1H-pyrrolo[1,2-a]indol-2-yl)carbamate

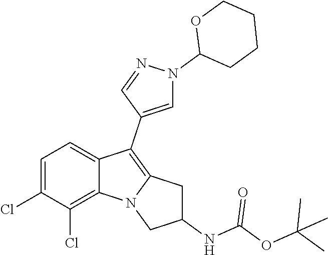 [SMILES: CC(C)(C)OC(=O)NC1Cc2c(-c3cnn(C4CCCCO4)c3)c3ccc(Cl)c(Cl)c3n2C1]

Dissolve tert-butyl (5,6-dichloro-9-iodo-2,3-dihydro-1H-pyrrolo[1,2-a]indol-2-yl)carbamate (150 mg, 0.289 mmol), 1-(tetrahydro-2H-pyran-2-yl)-4-(4,4,5,5-tetramethyl-1,3,2-dioxaborolan-2-yl)-1H-pyrazole (142 mg, 0.485 mmol), Pd(dppf)Cl₂ (24 mg, 0.032 mmol) and sodium carbonate (70 mg, 0.66 mmol) in 1,4-dioxane (8 mL) and water (2 mL). Purge with N₂ and stir at 90° C. for 6 hrs., under N₂ atmosphere. Combine with another batch run at 0.37×scale for work up and purification. Concentrate under vacuum and purify by flash column chromatography eluting with EtOAc in petroleum ether to give tert-butyl (5,6-dichloro-9-(1-(tetrahydro-2H-pyran-2-yl)-1H-pyrazol-4-yl)-2,3-dihydro-1H-pyrrolo[1,2-a]indol-2-yl)carbamate (170 mg, 79%) as a yellow oil. ES/MS (m/z): (³⁵Cl/³⁷Cl) 491.0/493.0 (M+H).

### Intermediate 392

### 5,6-Dichloro-9-(1H-pyrazol-4-yl)-2,3-dihydro-1H-pyrrolo[1,2-a]indol-2-amine

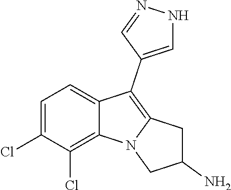 [SMILES: NC1Cc2c(-c3cn[nH]c3)c3ccc(Cl)c(Cl)c3n2C1]

Dissolve tert-butyl (5,6-dichloro-9-(1-(tetrahydro-2H-pyran-2-yl)-1H-pyrazol-4-yl)-2,3-dihydro-1H-pyrrolo[1,2-a]indol-2-yl)carbamate (170 mg, 0.350 mmol) in 4M HCl in MeOH (15 mL). Stir at ambient temperature for 1 hr. Concentrate to give 5,6-dichloro-9-(1H-pyrazol-4-yl)-2,3-dihydro-1H-pyrrolo[1,2-a]indol-2-amine (100 mg, 85%, HCl salt) as a green solid. ES/MS (m/z): (³⁵Cl/³⁷Cl) 307.1/309.1 (M+H).

### EXAMPLE 154

### N-(5,6-Dichloro-9-(1H-pyrazol-4-yl)-2,3-dihydro-1H-pyrrolo[1,2-a]indol-2-yl)-2-hydroxyacetamide

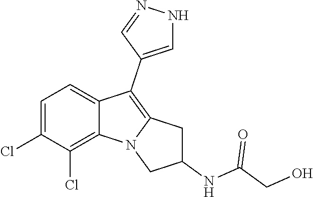 [SMILES: O=C(CO)NC1Cc2c(-c3cn[nH]c3)c3ccc(Cl)c(Cl)c3n2C1]

Dissolve 5,6-dichloro-9-(1H-pyrazol-4-yl)-2,3-dihydro-1H-pyrrolo[1,2-a]indol-2-amine (100 mg, 0.291 mmol, HCl salt), glycolic acid (75 mg, 0.99 mmol), DIPEA (0.57 mL, 3.27 mmol) and HATU (315 mg, 0.812 mmol) in DMF (3 mL, 38.8 mmol). Stir at ambient temperature for 16 hrs under N₂ atmosphere. Concentrate under vacuum and purify by prep-HPLC to give N-(5,6-dichloro-9-(1H-pyrazol-4-yl)-2,3-dihydro-1H-pyrrolo[1,2-a]indol-2-yl)-2-hydroxyacetamide (50.71 mg, 47%) as a white solid. ES/MS (m/z): (³⁵Cl/³⁷Cl) 365.3/367.2 (M+H).

### Intermediate 393

### N-(9-(1-Acetyl-1H-pyrazol-4-yl)-5,6-dichloro-2,3-dihydro-1H-pyrrolo[1,2-a]indol-2-yl)acetamide

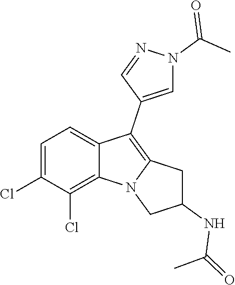 [SMILES: CC(=O)NC1Cc2c(-c3cnn(C(C)=O)c3)c3ccc(Cl)c(Cl)c3n2C1]

Dissolve 5,6-dichloro-9-(1H-pyrazol-4-yl)-2,3-dihydro-1H-pyrrolo[1,2-a]indol-2-amine (40 mg, 0.13 mmol) in pyridine (2 mL). Add acetic anhydride (0.026 mL, 0.28 mmol) and stir at ambient temperature for 16-18 hrs. Concentrate under vacuum to give N-(9-(1-acetyl-1H-pyrazol-4-yl)-5,6-dichloro-2,3-dihydro-1H-pyrrolo[1,2-a]indol-2-yl)acetamide (50 mg, crude) as a green solid. ES/MS (m/z): (³⁵Cl/³⁷Cl) 391.0/393.0 (M+H).

### EXAMPLE 155

### N-(5, 6-Dichloro-9-(1H-pyrazol-4-yl)-2,3-dihydro-1H-pyrrolo[1,2-a]indol-2-yl)acetamide

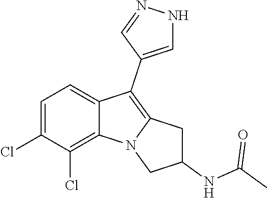 [SMILES: CC(=O)NC1Cc2c(-c3cn[nH]c3)c3ccc(Cl)c(Cl)c3n2C1]

Suspend N-(9-(1-acetyl-1H-pyrazol-4-yl)-5,6-dichloro-2,3-dihydro-1H-pyrrolo[1,2-a]indol-2-yl)acetamide (40 mg, 0.10 mmol) and K₂CO₃ (180 mg, 1.30 mmol) in MeOH (5 mL). Stir at ambient temperature for 16-18 hrs. Filter and concentrate the filtrate. Purify by prep-HPLC to give N-(5,6-dichloro-9-(1H-pyrazol-4-yl)-2,3-dihydro-1H-pyrrolo[1,2-a]indol-2-yl)acetamide (8.41 mg, 0.0239 mmol, 23%) as a yellow solid. ES/MS (m/z): (³⁵Cl/³⁷Cl) 349.2/351.2 (M+H).

### Intermediate 394

### N-[[6,7-Dichloro-3-(1-tetrahydropyran-2-ylpyrazol-4-yl)-1H-indole-2-carbonyl]amino]formamide

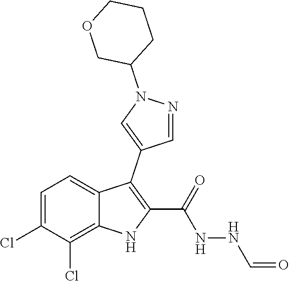 [SMILES: O=CNNC(=O)c1[nH]c2c(Cl)c(Cl)ccc2c1-c1cnn(C2CCCOC2)c1]

Dissolve 6,7-dichloro-3-(1-tetrahydropyran-2-ylpyrazol-4-yl)-1H-indole-2-carboxylic acid (500 mg, 1.18 mmol), formic hydrazide (218 mg, 3.56 mmol) and DIPEA (1.03 mL, 5.91 mmol) in DMF (8 mL). Add HATU (920 mg, 2.40 mmol). Stir at ambient temperature under N₂ for 16 hrs. Dilute with water and extract with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give N-[[6,7-dichloro-3-(1-tetrahydropyran-2-ylpyrazol-4-yl)-1H-indole-2-carbonyl]amino]formamide (488 mg, 87%) as a yellow solid. ES/MS (m/z): (³⁵Cl/³⁷Cl) 422.1/424.0 (M+H).

### Intermediate 395

### 2-[6,7-Dichloro-3-(1-tetrahydropyran-2-ylpyrazol-4-yl)-1H-indol-2-yl]-1,3,4-oxadiazole

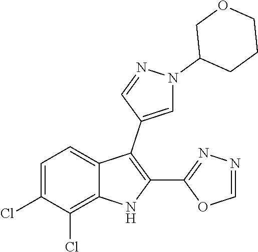 [SMILES: Clc1ccc2c(-c3cnn(C4CCCOC4)c3)c(-c3nnco3)[nH]c2c1Cl]

Dissolve N-[[6,7-dichloro-3-(1-tetrahydropyran-2-ylpyrazol-4-yl)-1H-indole-2-carbonyl]amino]formamide (488 mg, 1.02 mmol) in CH₃CN (10 mL) and add DIPEA (0.54 mL, 3.1 mmol). Purge with N₂ and cool to 0° C. Add p-toluenesulfonyl chloride (411 mg, 2.05 mmol) and stir at ambient temperature for 16 hrs. Dilute with EtOAc, wash with saturated aqueous NaCl, dry over with anhydrous Na₂SO₄, filter, and concentrate under vacuum. Purify by flash column chromatography eluting with EtOAc in petroleum ether to give 2-[6,7-dichloro-3-(1-tetrahydropyran-2-ylpyrazol-4-yl)-1H-indol-2-yl]-1,3,4-oxadiazole (240 mg, 48%) as a yellow solid. ES/MS (m/z): (³⁵Cl/³⁷Cl) 404.1/406.0 (M+H).

### EXAMPLE 156

### 2-[6,7-Dichloro-3-(1H-pyrazol-4-yl)-1H-indol-2-yl]-1,3,4-oxadiazole

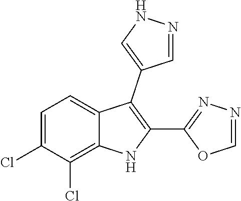 [SMILES: Clc1ccc2c(-c3cn[nH]c3)c(-c3nnco3)[nH]c2c1Cl]

Dissolve 2-[6,7-dichloro-3-(1-tetrahydropyran-2-ylpyrazol-4-yl)-1H-indol-2-yl]-1,3,4-oxadiazole (210 mg, 0.426 mmol) in formic acid (4 mL) and stir at ambient temperature for 2 hrs. Concentrate under vacuum and purify by prep-HPLC to give 2-[6,7-dichloro-3-(1H-pyrazol-4-yl)-1H-indol-2-yl]-1,3,4-oxadiazole (18.19 mg, 13%) as a white solid. ES/MS (m/z): (³⁵Cl/³⁷Cl) 320.2/322.2 (M+H).

### Intermediate 396

### Methyl 2-[2-[6,7-dichloro-3-(1H-pyrazol-4-yl)-1H-indole-2-carbonyl]hydrazino]-2-oxo-acetate

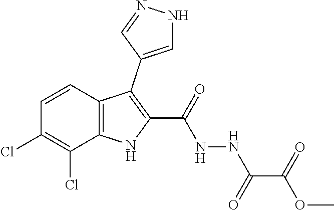 [SMILES: COC(=O)C(=O)NNC(=O)c1[nH]c2c(Cl)c(Cl)ccc2c1-c1cn[nH]c1]

Dissolve 6,7-dichloro-3-(1-tetrahydropyran-2-ylpyrazol-4-yl)-1H-indole-2-carbohydrazide (250 mg, 0.609 mmol) and TEA (0.11 mL, 0.79 mmol) in DCM (10 mL). Cool to 0° C. and add methyl oxalyl chloride (100 mg, 0.816 mmol). Stir at ambient temperature for 16 hrs. Dilute with water, extract with EtOAc (2×), wash with saturated aqueous NaCl, dry over anhydrous sodium sulfate, filter, and concentrate under vacuum. Purify by flash column chromatography eluting with EtOAc in petroleum ether to give methyl 2-[2-[6,7-dichloro-3-(1H-pyrazol-4-yl)-1H-indole-2-carbonyl]hydrazino]-2-oxo-acetate (220 mg, 90%) as a yellow solid. ES/MS (m/z): (³⁵Cl/³⁷Cl) 395.9/397.9 (M+H).

### Intermediate 397

### Methyl 5-[6,7-dichloro-3-(1H-pyrazol-4-yl)-1H-indol-2-yl]-1,3,4-oxadiazole-2-carboxylate

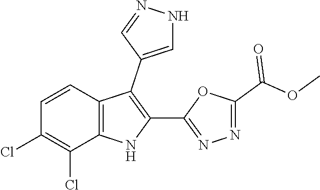 [SMILES: COC(=O)c1nnc(-c2[nH]c3c(Cl)c(Cl)ccc3c2-c2cn[nH]c2)o1]

Dissolve methyl 2-[2-[6,7-dichloro-3-(1H-pyrazol-4-yl)-1H-indole-2-carbonyl]hydrazino]-2-oxo-acetate (220 mg, 0.550 mmol) in CH₃CN (10 mL). Add DIPEA (220 mg, 1.70 mmol) and p-toluenesulfonyl chloride (224 mg, 1.12 mmol) at 0° C. under N₂ atmosphere, then stir at ambient temperature for 16 hrs. Collect the solids by filtration to give methyl 5-[6,7-dichloro-3-(1H-pyrazol-4-yl)-1H-indol-2-yl]-1,3,4-oxadiazole-2-carboxylate (220 mg, 95%) as a yellow solid.

### EXAMPLE 157

### (5-(6, 7-Dichloro-3-(1H-pyrazol-4-yl)-1H-indol-2-yl)-1,3,4-oxadiazol-2-yl)methanol

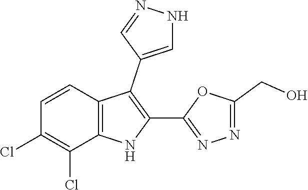 [SMILES: OCc1nnc(-c2[nH]c3c(Cl)c(Cl)ccc3c2-c2cn[nH]c2)o1]

Dissolve methyl 5-[6,7-dichloro-3-(1H-pyrazol-4-yl)-1H-indol-2-yl]-1,3,4-oxadiazole-2-carboxylate (220 mg, 0.523 mmol) in DCM (10 mL). Add DIBAL-H (2.9 mL, 2.90 mmol, 1.0M in toluene) dropwise at 0° C. under a N₂ atmosphere. Stir at ambient temperature for 16-18 hrs. Quench with saturated aqueous potassium sodium tartrate, dilute with water, extract with EtOAc, wash with saturated aqueous NaCl, dry over anhydrous sodium sulfate, filter, and concentrate under vacuum. Purify by flash column chromatography eluting with EtOAc in petroleum ether, then triturate with CH₃CN to give (5-(6,7-dichloro-3-(1H-pyrazol-4-yl)-1H-indol-2-yl)-1,3,4-oxadiazol-2-yl)methanol (13.95 mg, 7%) as a yellow solid. ES/MS (m/z): (³⁵Cl/³⁷Cl) 350.2/352.2 (M+H).

### EXAMPLE 158

### 2-(6,7-Dichloro-3-(1H-pyrazol-4-yl)-1H-indol-2-yl)-5-methyl-1,3,4-thiadiazole

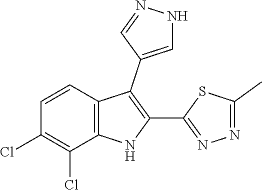 [SMILES: Cc1nnc(-c2[nH]c3c(Cl)c(Cl)ccc3c2-c2cn[nH]c2)s1]

Dissolve N′-acetyl-6,7-dichloro-3-(1-tetrahydropyran-2-ylpyrazol-4-yl)-1H-indole-2-carbohydrazide (300 mg, 0.619 mmol) and Lawesson's reagent (300 mg, 0.720 mmol) in toluene (10 mL). Stir at 110° C. for 2 hrs., under N₂ atmosphere. Concentrate under vacuum and purify by prep-HPLC to give 2-(6,7-dichloro-3-(1H-pyrazol-4-yl)-1H-indol-2-yl)-5-methyl-1,3,4-thiadiazole (22.26 mg, 10%) as a yellow solid. ES/MS (m/z): (³⁵Cl/³⁷Cl) 350.2/352.2 (M+H).

### Intermediate 398

### [[6,7-Dichloro-3-(1-tetrahydropyran-2-ylpyrazol-4-yl)-1H-indole-2-carbonyl]amino]thiourea

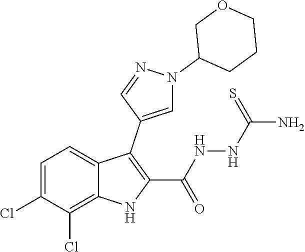 [SMILES: NC(=S)NNC(=O)c1[nH]c2c(Cl)c(Cl)ccc2c1-c1cnn(C2CCCOC2)c1]

Dissolve 6,7-dichloro-3-(1-tetrahydropyran-2-ylpyrazol-4-yl)-1H-indole-2-carboxylic acid (150 mg, 0.355 mmol), hydrazinecarbothioamide (97 mg, 1.1 mmol) and DIPEA (0.31 mL, 1.8 mmol), HATU (276 mg, 0.711 mmol) in DMSO (3 mL). Purge with N₂. Stir at ambient temperature under N₂ for 16 hrs. Dilute with water and extract the aqueous layer with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give [[6,7-dichloro-3-(1-tetrahydropyran-2-ylpyrazol-4-yl)-1H-indole-2-carbonyl]amino]thiourea (120 mg, 57%) as a yellow oil. ES/MS (m/z): (³⁵Cl/³⁷Cl) 453.1/454.8 (M+H) and 369.1/371.0 (M+H-THP).

### Intermediate 399

### 5-[6,7-Dichloro-3-(1-tetrahydropyran-2-ylpyrazol-4-yl)-1H-indol-2-yl]-4H-1,2,4-triazole-3-thiol

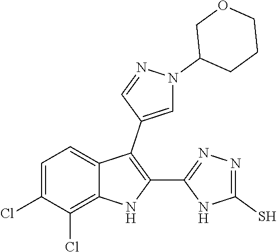 [SMILES: Sc1nnc(-c2[nH]c3c(Cl)c(Cl)ccc3c2-c2cnn(C3CCCOC3)c2)[nH]1]

Dissolve [[6,7-dichloro-3-(1-tetrahydropyran-2-ylpyrazol-4-yl)-1H-indole-2-carbonyl]amino]thiourea (120 mg, 0.203 mmol), in 4M aqueous KOH (3 mL). Purge with N₂ and stir at 100° C. for 3 hrs. Acidify with 2N aqueous HCl to pH=3 and extract with EtOAc, wash with saturated aqueous NaCl, dry over anhydrous Na₂SO₄, filter, and concentrate under vacuum to give 5-[6,7-dichloro-3-(1-tetrahydropyran-2-ylpyrazol-4-yl)-1H-indol-2-yl]-4H-1,2,4-triazole-3-thiol (100 mg, 83%) as a yellow solid. ES/MS (m/z): (³⁵Cl/³⁷Cl) 435.1/437.0 (M+H).

### EXAMPLE 159

### 5-[6,7-Dichloro-3-(1H-pyrazol-4-yl)-1H-indol-2-yl]-4H-1,2,4-triazole-3-thiol

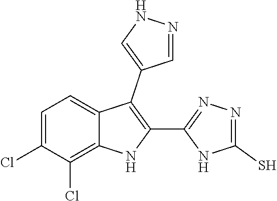 [SMILES: Sc1nnc(-c2[nH]c3c(Cl)c(Cl)ccc3c2-c2cn[nH]c2)[nH]1]

Dissolve 5-[6,7-dichloro-3-(1-tetrahydropyran-2-ylpyrazol-4-yl)-1H-indol-2-yl]-4H-1,2,4-triazole-3-thiol (85 mg, 0.18 mmol) in THF (2 mL). Add 6M aqueous HCl (5 mL) and stir at ambient temperature for 6 hrs. Concentrate under vacuum and purify by trituration with CH₃CN to give 5-[6,7-dichloro-3-(1H-pyrazol-4-yl)-1H-indol-2-yl]-4H-1,2,4-triazole-3-thiol (42.26 mg, 65%) as a white solid. ES/MS (m/z): (³⁵Cl/³⁷Cl) 351.2/353.2 (M+H).

### Intermediate 400

### 6,7-Dichloro-N′-(cyclopropanecarbonyl)-3-(1-(tetrahydro-2H-pyran-2-yl)-1H-pyrazol-4-yl)-1H-indole-2-carbohydrazide

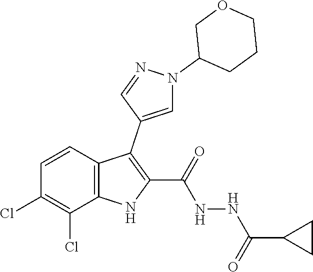 [SMILES: O=C(NNC(=O)C1CC1)c1[nH]c2c(Cl)c(Cl)ccc2c1-c1cnn(C2CCCOC2)c1]

Dissolve 6,7-dichloro-3-(1-tetrahydropyran-2-ylpyrazol-4-yl)-1H-indole-2-carboxylic acid (500 mg, 1.18 mmol), cyclopropanecarbohydrazide (241 mg, 2.41 mmol) and DIPEA (1.15 mL, 6.59 mmol) in DMF (10 mL). Add HATU (1.02 g, 2.63 mmol) and stir at ambient temperature for 16 hrs. Dilute with water, extract with EtOAc, wash with saturated aqueous NaCl, dry over anhydrous sodium sulfate, filter, and concentrate under vacuum. Purify by flash column chromatography eluting with EtOAc in petroleum ether to give 6,7-dichloro-N′-(cyclopropanecarbonyl)-3-(1-(tetrahydro-2H-pyran-2-yl)-1H-pyrazol-4-yl)-1H-indole-2-carbohydrazide (600 mg, 99%) as a yellow solid.

### Intermediate 401

### 2-Cyclopropyl-5-(6,7-dichloro-3-(1-(tetrahydro-2H-pyran-2-yl)-1H-pyrazol-4-yl)-1H-indol-2-yl)-1,3,4-oxadiazole

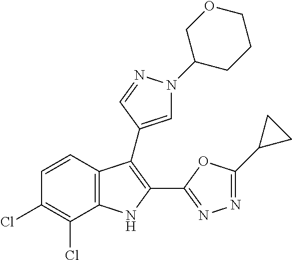 [SMILES: Clc1ccc2c(-c3cnn(C4CCCOC4)c3)c(-c3nnc(C4CC4)o3)[nH]c2c1Cl]

Dissolve 6,7-dichloro-N′-(cyclopropanecarbonyl)-3-(1-(tetrahydro-2H-pyran-2-yl)-1H-pyrazol-4-yl)-1H-indole-2-carbohydrazide (200 mg, 0.389 mmol) in CH₃CN (10 mL). Add DIPEA (167 mg, 1.29 mmol) and p-toluenesulfonyl chloride (175 mg, 0.872 mmol) at 0° C. under N₂ atmosphere. Stir at ambient temperature for 16 hrs. Dilute with water, extract with EtOAc, wash with saturated aqueous NaCl, dry over anhydrous sodium sulfate, filter, and concentrate under vacuum to give 2-cyclopropyl-5-(6,7-dichloro-3-(1-(tetrahydro-2H-pyran-2-yl)-1H-pyrazol-4-yl)-1H-indol-2-yl)-1,3,4-oxadiazole (150 mg, 86%) as a yellow solid. ES/MS (m/z): (³⁵Cl/³⁷Cl) 444.3/446.2 (M+H, small peak) and 360.3/362.2 (M+H-THP).

### EXAMPLE 160

### 2-Cyclopropyl-5-(6,7-dichloro-3-(1H-pyrazol-4-yl)-1H-indol-2-yl)-1,3,4-oxadiazole

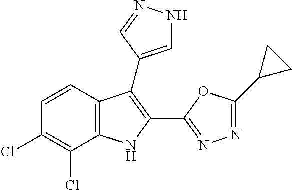 [SMILES: Clc1ccc2c(-c3cn[nH]c3)c(-c3nnc(C4CC4)o3)[nH]c2c1Cl]

Dissolve 2-cyclopropyl-5-(6,7-dichloro-3-(1-(tetrahydro-2H-pyran-2-yl)-1H-pyrazol-4-yl)-1H-indol-2-yl)-1,3,4-oxadiazole (150 mg, 0.303 mmol) in DCM (2 mL) and add TFA (2 mL). Stir at ambient temperature for 2 hrs. Concentrate under vacuum and purify by prep-HPLC to give 2-cyclopropyl-5-(6,7-dichloro-3-(1H-pyrazol-4-yl)-1H-indol-2-yl)-1,3,4-oxadiazole (33.98 mg, 31%) as a white solid. ES/MS (m/z): (³⁵Cl/³⁷Cl) 360.2/362.2 (M+H).

### Intermediate 402

### 2-(6-Chloro-4-methoxy-indol-1-yl)ethynyl-triisopropylsilane

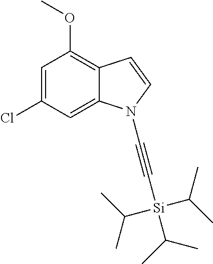 [SMILES: COc1cc(Cl)cc2c1ccn2C#C[Si](C(C)C)(C(C)C)C(C)C]

Dissolve 6-chloro-4-methoxy-1H-indole (1.00 g, 5.34 mmol) in 1,4-dioxane (20 mL). Add (bromoethynyl)triisopropylsilane (7.19 g, 26.7 mmol), PEG-400 (200 mg), CuI (102 mg, 0.536 mmol) and Cs₂CO₃ (2.08 g, 6.38 mmol). Purge with N₂ stir at 135° C. under N₂ for 16 hrs. Dilute with water and extract the aqueous layer with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give 2-(6-chloro-4-methoxy-indol-1-yl)ethynyl-triisopropylsilane (1.26 g, 59%) as a yellow oil. ES/MS (m/z): (³⁵Cl/³⁷Cl) 362.2/364.2 (M+H).

### Intermediate 403

### 6-Chloro-1-ethynyl-4-methoxy-indole

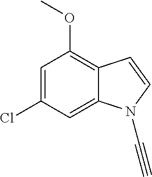

Dissolve 2-(6-chloro-4-methoxy-indol-1-yl)ethynyl-triisopropylsilane (560 mg, 1.39 mmol) in THF (5 mL) and add TBAF (5 mL, 1M in THF). Stir at ambient temperature for 2 hrs. Dilute with EtOAc, wash with saturated aqueous NH₄Cl, dry over anhydrous Na₂SO₄, filter, and concentrate under vacuum. Purify by flash column chromatography eluting with EtOAc in petroleum ether to give 6-chloro-1-ethynyl-4-methoxy-indole (295 mg, 93%) as a brown solid.

### Intermediate 404

### tert-Butyl-[2-[4-(6-chloro-4-methoxy-indol-1-yl)triazol-1-yl]ethoxy]-dimethyl-silane

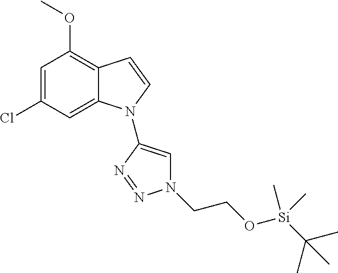 [SMILES: COc1cc(Cl)cc2c1ccn2-c1cn(CCO[Si](C)(C)C(C)(C)C)nn1]

Dissolve 6-chloro-1-ethynyl-4-methoxy-indole (295 mg, 1.29 mmol) in DMF (4 mL) and H₂O (4 mL). Add copper (17 mg, 0.28 mmol), CuSO₄ (44 mg, 0.28 mmol) and (2-azidoethoxy)(tert-butyl)dimethylsilane (347 mg, 1.64 mmol). Purge with N₂. Stir at 110° C. under N₂ for 0.5 hr. Dilute with water and extract the aqueous layer with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give tert-butyl-[2-[4-(6-chloro-4-methoxy-indol-1-yl)triazol-1-yl]ethoxy]-dimethyl-silane (350 mg, 60%) as a yellow oil. ES/MS (m/z): (³⁵Cl/³⁷Cl) 407.2/409.2 (M+H).

### Intermediate 405

### tert-Butyl-[2-[4-(6-chloro-3-iodo-4-methoxy-indol-1-yl)triazol-1-yl]ethoxy]-dimethyl-silane

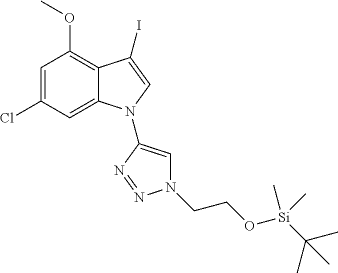 [SMILES: COc1cc(Cl)cc2c1c(I)cn2-c1cn(CCO[Si](C)(C)C(C)(C)C)nn1]

Dissolve tert-butyl-[2-[4-(6-chloro-4-methoxy-indol-1-yl)triazol-1-yl]ethoxy]-dimethyl-silane (250 mg, 0.553 mmol) in DMF (3 mL) and add NIS (153 mg, 0.666 mmol). Stir at ambient temperature for 2 hrs. Quench with saturated aqueous Na₂SO₃ and extract the aqueous layer with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give tert-butyl-[2-[4-(6-chloro-3-iodo-4-methoxy-indol-1-yl)triazol-1-yl]ethoxy]-dimethyl-silane (180 mg, 58%) as a yellow oil.

### Intermediate 406

### 1-(1-(2-((tert-Butyldimethylsilyl)oxy)ethyl)-1H-1,2,3-triazol-4-yl)-6-chloro-4-methoxy-3-(1-(tetrahydro-2H-pyran-2-yl)-1H-pyrazol-4-yl)-1H-indole

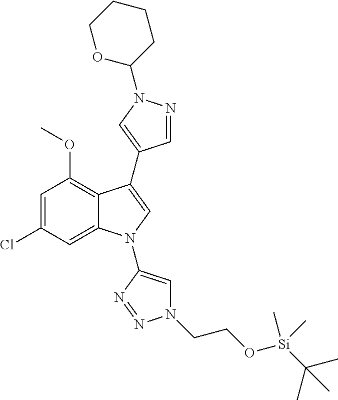 [SMILES: COc1cc(Cl)cc2c1c(-c1cnn(C3CCCCO3)c1)cn2-c1cn(CCO[Si](C)(C)C(C)(C)C)nn1]

Dissolve tert-butyl-[2-[4-(6-chloro-3-iodo-4-methoxy-indol-1-yl)triazol-1-yl]ethoxy]-dimethyl-silane (150 mg, 0.267 mmol) in 1,4-dioxane (4 mL) and H₂O (1 mL). Add Na₂CO₃ (86 mg, 0.80 mmol), Pd(dtbpf)Cl₂ (27 mg, 0.041 mmol) and 1-(tetrahydro-2H-pyran-2-yl)-4-(4,4,5,5-tetramethyl-1,3,2-dioxaborolan-2-yl)-1H-pyrazole (118 mg, 0.403 mmol). Purge with N₂. Stir at 90° C. under N₂ for 2 hrs. Combine with another batch run at 0.20×scale for work up and purification. Dilute with water and extract the aqueous layer with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give 1-(1-(2-((tert-butyldimethylsilyl)oxy)ethyl)-1H-1,2,3-triazol-4-yl)-6-chloro-4-methoxy-3-(1-(tetrahydro-2H-pyran-2-yl)-1H-pyrazol-4-yl)-1H-indole (160 mg, 81%) as a yellow solid. ES/MS (m/z): (³⁵Cl/³⁷Cl) 557.3 (M+H) and 473.2/475.2 (M+H-THP).

### EXAMPLE 161

### 2-[4-[6-Chloro-4-methoxy-3-(1H-pyrazol-4-yl)indol-1-yl]triazol-1-yl]ethanol

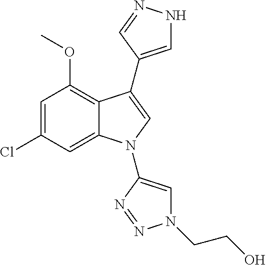 [SMILES: COc1cc(Cl)cc2c1c(-c1cn[nH]c1)cn2-c1cn(CCO)nn1]

Dissolve 1-(1-(2-((tert-butyldimethylsilyl)oxy)ethyl)-1H-1,2,3-triazol-4-yl)-6-chloro-4-methoxy-3-(1-(tetrahydro-2H-pyran-2-yl)-1H-pyrazol-4-yl)-1H-indole (160 mg, 0.261 mmol) in THF (1 mL) and add 6M aqueous HCl (3 mL). Stir at ambient temperature for 0.5 hr. Concentrate under vacuum and purify by prep-HPLC to give 2-[4-[6-chloro-4-methoxy-3-(1H-pyrazol-4-yl)indol-1-yl]triazol-1-yl]ethanol (63.14 mg, 67%) as a white solid. ES/MS (m/z): (³⁵Cl/³⁷Cl) 359.3/361.3 (M+H).

### Intermediate 407

### N-[[6,7-Dichloro-3-(1-tetrahydropyran-2-ylpyrazol-4-yl)-1H-indol-2-yl]methyl]-2-hydroxy-acetamide

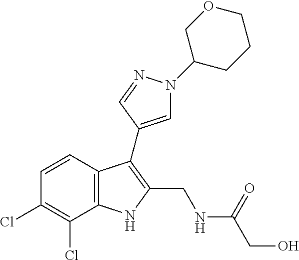 [SMILES: O=C(CO)NCc1[nH]c2c(Cl)c(Cl)ccc2c1-c1cnn(C2CCCOC2)c1]

Dissolve [6,7-dichloro-3-(1-tetrahydropyran-2-ylpyrazol-4-yl)-1H-indol-2-yl]methanamine (150 mg, 0.376 mmol), glycolic acid (60 mg, 0.79 mmol) and DIPEA (252 mg, 1.95 mmol) in DMF (4 mL) and add HATU (302 mg, 0.778 mmol). Stir at ambient temperature under N₂ for 16 hrs. Filter and concentrate the filtrate under vacuum. Dilute with water and extract the aqueous layer with DCM (3×). Wash the combined organic layers with saturated aqueous NaCl, dry over anhydrous sodium sulfate, filter, and concentrate under vacuum. Purify by flash column chromatography eluting with MeOH in DCM to give N-[[6,7-dichloro-3-(1-tetrahydropyran-2-ylpyrazol-4-yl)-1H-indol-2-yl]methyl]-2-hydroxy-acetamide (170 mg, 86%) as a white solid. ES/MS (m/z): (³⁵Cl/³⁷Cl) 423.1/425.0 (M+H).

### EXAMPLE 162

### N-[[6,7-Dichloro-3-(1H-pyrazol-4-yl)-1H-indol-2-yl]methyl]-2-hydroxy-acetamide

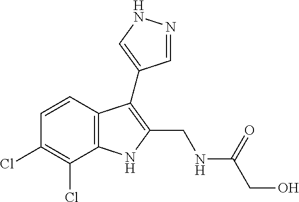 [SMILES: O=C(CO)NCc1[nH]c2c(Cl)c(Cl)ccc2c1-c1cn[nH]c1]

Dissolve N-[[6,7-dichloro-3-(1-tetrahydropyran-2-ylpyrazol-4-yl)-1H-indol-2-yl]methyl]-2-hydroxy-acetamide (170 mg, 0.321 mmol) in THF (3 mL) and add 6M aqueous HCl (4 mL). Stir at ambient temperature for 2 hrs. Concentrate under vacuum and purify by prep-HPLC to give N-[[6,7-dichloro-3-(1H-pyrazol-4-yl)-1H-indol-2-yl]methyl]-2-hydroxy-acetamide (98.75 mg, 91%) as a white solid. ES/MS (m/z): (³⁵Cl/³⁷Cl) 339.3/341.3 (M+H).

### EXAMPLE 163

### N-[[6,7-Dichloro-3-(1H-pyrazol-4-yl)-1H-indol-2-yl]methyl]propanamide

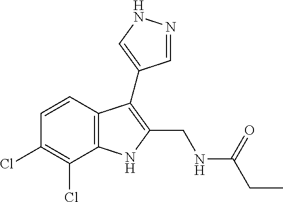 [SMILES: CCC(=O)NCc1[nH]c2c(Cl)c(Cl)ccc2c1-c1cn[nH]c1]

Dissolve [6,7-dichloro-3-(1-tetrahydropyran-2-ylpyrazol-4-yl)-1H-indol-2-yl]methanamine (150 mg, 0.376 mmol) and TEA (0.11 mL, 0.79 mmol) in DCM (4 mL). Add propionyl chloride (0.07 mL, 0.80 mmol) at 0° C. under N₂ and stir at ambient temperature for 2 hrs. Concentrate under vacuum and dissolve the residue in MeOH (3 mL), add K₂CO₃ (142 mg, 1.03 mmol), then stir at ambient temperature for 2 hrs. Dilute with water, extract with EtOAc, wash with saturated aqueous NaCl, dry over with anhydrous Na₂SO₄, filter, and concentrate under vacuum. Purify by prep-HPLC to give N-[[6,7-dichloro-3-(1H-pyrazol-4-yl)-1H-indol-2-yl]methyl]propanamide (74.44 mg, 65%) as a white solid. ES/MS (m/z): (³⁵Cl/³⁷Cl) 337.3/339.3 (M+H).

### Intermediate 408

### [6,7-Dichloro-3-(1-tetrahydropyran-2-ylpyrazol-4-yl)-1H-indol-2-yl]methylurea

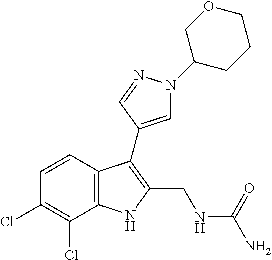 [SMILES: NC(=O)NCc1[nH]c2c(Cl)c(Cl)ccc2c1-c1cnn(C2CCCOC2)c1]

Dissolve [6,7-dichloro-3-(1-tetrahydropyran-2-ylpyrazol-4-yl)-1H-indol-2-yl]methanamine (200 mg, 0.509 mmol) in MeOH (4 mL). Add KOCN (126 mg, 1.52 mmol) and acetic acid (305 mg, 5.08 mmol). Stir at 65° C. for 2 hrs. Dilute with EtOAc and water and separate the layers. Extract the aqueous layer with EtOAc (3×). Wash the combined organic layers with saturated aqueous NaCl, dry over anhydrous sodium sulfate, filter, and concentrate under vacuum to give [6,7-dichloro-3-(1-tetrahydropyran-2-ylpyrazol-4-yl)-1H-indol-2-yl]methylurea (250 mg, 89%) as a yellow solid. ES/MS (m/z): (³⁵Cl/³⁷Cl) 408.1/410.0 (M+H).

### EXAMPLE 164

### [6,7-Dichloro-3-(1H-pyrazol-4-yl)-1H-indol-2-yl]methylurea

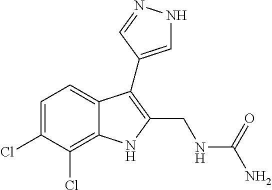 [SMILES: NC(=O)NCc1[nH]c2c(Cl)c(Cl)ccc2c1-c1cn[nH]c1]

Dissolve [6,7-dichloro-3-(1-tetrahydropyran-2-ylpyrazol-4-yl)-1H-indol-2-yl]methylurea (250 mg, 0.453 mmol) in 4M HCl in MeOH (5 mL) and stir at ambient temperature for 2 hrs. Concentrate under vacuum and purify by trituration with MeOH and acetonitrile to give [6,7-dichloro-3-(1H-pyrazol-4-yl)-1H-indol-2-yl]methylurea (57.88 mg, 39%) as a white solid. ES/MS (m/z): (³⁵Cl/³⁷Cl) 324.2/326.2 (M+H).

### Intermediate 409

### N-[[6,7-Dichloro-3-(1-tetrahydropyran-2-ylpyrazol-4-yl)-1H-indol-2-yl]methyl]-1,3,4-oxadiazole-2-carboxamide

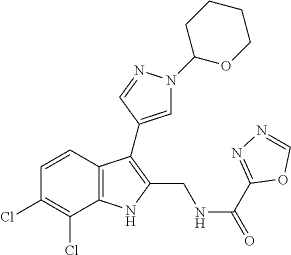 [SMILES: O=C(NCc1[nH]c2c(Cl)c(Cl)ccc2c1-c1cnn(C2CCCCO2)c1)c1nnco1]

Dissolve [6,7-dichloro-3-(1-tetrahydropyran-2-ylpyrazol-4-yl)-1H-indol-2-yl]methanamine (150 mg, 0.329 mmol) and methyl 1,3,4-oxadiazole-2-carboxylate (240 mg, 1.69 mmol) in toluene (10 mL). Add Me₃Al (1.1 mL, 2.2 mmol, 2M in toluene) dropwise. Stir at 80° C. for 16 hrs., under N₂ atmosphere. Quench with water, extract with EtOAc (2×), wash the combined organic layers with saturated aqueous NaCl, dry over anhydrous sodium sulfate, filter, and concentrate under vacuum. Purify by flash column chromatography eluting with EtOAc in petroleum ether to give N-[[6,7-dichloro-3-(1-tetrahydropyran-2-ylpyrazol-4-yl)-1H-indol-2-yl]methyl]-1,3,4-oxadiazole-2-carboxamide (40 mg, 25%) as a yellow oil. ES/MS (m/z): (³⁵Cl/³⁷Cl) 461.0/463.0 (M+H).

### EXAMPLE 165

### N-[[6,7-Dichloro-3-(1H-pyrazol-4-yl)-1H-indol-2-yl]methyl]-1,3,4-oxadiazole-2-carboxamide

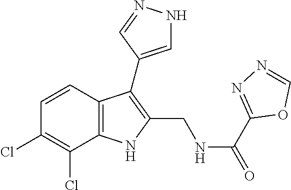 [SMILES: O=C(NCc1[nH]c2c(Cl)c(Cl)ccc2c1-c1cn[nH]c1)c1nnco1]

Dissolve N-[[6,7-dichloro-3-(1-tetrahydropyran-2-ylpyrazol-4-yl)-1H-indol-2-yl]methyl]-1,3,4-oxadiazole-2-carboxamide (40 mg, 0.075 mmol) in formic acid (2 mL). Stir at ambient temperature for 2 hrs. Quench with saturated aqueous Na₂CO₃, extract with EtOAc, wash with saturated aqueous NaCl, dry over anhydrous sodium sulfate, filter, and concentrate under vacuum. Purify by prep-HPLC to give N-[[6,7-dichloro-3-(1H-pyrazol-4-yl)-1H-indol-2-yl]methyl]-1,3,4-oxadiazole-2-carboxamide (3.20 mg, 11%) as a white solid. ES/MS (m/z): (³⁵Cl/³⁷Cl) 377.2/379.2 (M+H).

### Intermediate 410

### Ethyl 6,7-dichloro-3-(1-(tetrahydro-2H-pyran-2-yl)-1H-pyrazol-4-yl)-1-((triisopropylsilyl)ethynyl)-1H-indole-2-carboxylate

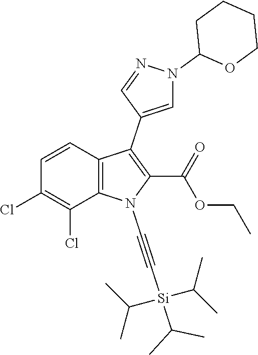 [SMILES: CCOC(=O)c1c(-c2cnn(C3CCCCO3)c2)c2ccc(Cl)c(Cl)c2n1C#C[Si](C(C)C)(C(C)C)C(C)C]

Dissolve ethyl 6,7-dichloro-3-(1-tetrahydropyran-2-ylpyrazol-4-yl)-1H-indole-2-carboxylate (2.00 g, 3.92 mmol) in 1,4-dioxane (30 mL). Add (bromoethynyl)triisopropylsilane (5.3 g, 20 mmol), PEG-400 (600 mg), CuI (75 mg, 0.39 mmol) and Cs₂CO₃ (1.5 g, 4.6 mmol). Purge with N₂ and stir at 130° C. under N₂ for 16 hrs. Dilute with water and extract the aqueous layer with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give ethyl 6,7-dichloro-3-(1-(tetrahydro-2H-pyran-2-yl)-1H-pyrazol-4-yl)-1-((triisopropylsilyl)ethynyl)-1H-indole-2-carboxylate (315 mg, 10%) as a yellow oil.

### Intermediate 411

### Ethyl 6, 7-dichloro-1-ethynyl-3-(1-(tetrahydro-2H-pyran-2-yl)-1H-pyrazol-4-yl)-1H-indole-2-carboxylate

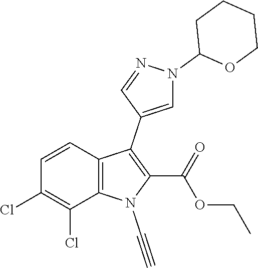 [SMILES: C#Cn1c(C(=O)OCC)c(-c2cnn(C3CCCCO3)c2)c2ccc(Cl)c(Cl)c21]

Dissolve ethyl 6,7-dichloro-3-(1-(tetrahydro-2H-pyran-2-yl)-1H-pyrazol-4-yl)-1-((triisopropylsilyl)ethynyl)-1H-indole-2-carboxylate (515 mg, 0.613 mmol, 70 mass %) in THF (5 mL) and add TBAF (5 mL, 1M in THF). Stir at ambient temperature for 2 hrs. Dilute with EtOAc, wash with saturated aqueous NH₄Cl (3×), dry over with anhydrous Na₂SO₄, filter, and concentrate under vacuum. Purify by flash column chromatography eluting with EtOAc in petroleum ether to give ethyl 6,7-dichloro-1-ethynyl-3-(1-(tetrahydro-2H-pyran-2-yl)-1H-pyrazol-4-yl)-1H-indole-2-carboxylate (288 mg, 96%) as a yellow solid. ES/MS (m/z): (³⁵Cl/³⁷Cl) 432.1/434.0 (M+H).

### Intermediate 412

### Ethyl 6,7-dichloro-3-(1-(tetrahydro-2H-pyran-2-yl)-1H-pyrazol-4-yl)-1-(1-((trimethyl silyl)methyl)-1H-1,2,3-triazol -4-yl)-1H-indole-2-carboxylate

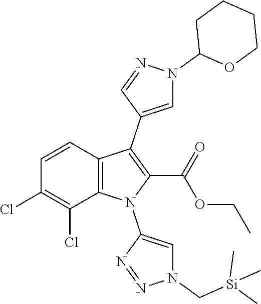 [SMILES: CCOC(=O)c1c(-c2cnn(C3CCCCO3)c2)c2ccc(Cl)c(Cl)c2n1-c1cn(C[Si](C)(C)C)nn1]

Dissolve ethyl 6,7-dichloro-1-ethynyl-3-(1-(tetrahydro-2H-pyran-2-yl)-1H-pyrazol-4-yl)-1H-indole-2-carboxylate (288 mg, 0.590 mmol) in DMF (5 mL) and H₂O (5 mL). Add copper (7 mg, 0.1 mmol), CuSO₄ (18 mg, 0.11 mmol) and trimethylsilylmethyl azide (94 mg, 0.71 mmol). Purge with N₂ and stir at 110° C. under N₂ for 0.5 hr. Dilute with water, extract with EtOAc (3×), wash the combined organic layers with saturated aqueous NaCl, dry over anhydrous sodium sulfate, filter, and concentrate under vacuum. Purify by flash column chromatography eluting with EtOAc in petroleum ether to give ethyl 6,7-dichloro-3-(1-(tetrahydro-2H-pyran-2-yl)-1H-pyrazol-4-yl)-1-(1-((trimethylsilyl)methyl)-1H-1,2,3-triazol-4-yl)-1H-indole-2-carboxylate (260 mg, 71%) as a yellow oil. ES/MS (m/z): (³⁵Cl/³⁷Cl) 561.2/563.1 (M+H).

### Intermediate 413

### (6, 7-Dichloro-3-(1-(tetrahydro-2H-pyran-2-yl)-1H-pyrazol-4-yl)-1-(1-((trimethyl silyl)methyl)-1H-1,2,3-triazol-4-yl)-1H-indol-2-yl)methanol

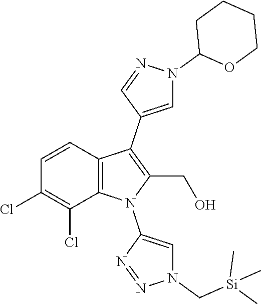 [SMILES: C[Si](C)(C)Cn1cc(-n2c(CO)c(-c3cnn(C4CCCCO4)c3)c3ccc(Cl)c(Cl)c32)nn1]

Dissolve ethyl 6, 7-dichloro-3-(1-(tetrahydro-2H-pyran-2-yl)-1H-pyrazol-4-yl)-1-(1-((trimethyl silyl)methyl)-1H-1,2,3-triazol -4-yl)-1H-indole-2-carboxylate (260 mg, 0.417 mmol) in DCM (6 mL). Cool to −78° C. and add DIBAL-H (3.3 mL, 3.3 mmol, 1.0M in toluene) dropwise. Stir at 0° C. for 3 hrs., under N₂ atmosphere. Quench with saturated aqueous sodium potassium tartrate, stir at ambient temperature for 2 hrs., dilute with water, extract with DCM (3×), wash the combined organic layers with saturated aqueous NaCl, dry over anhydrous Na₂SO₄, filter, and concentrate under vacuum. Purify by flash column chromatography eluting with EtOAc in petroleum ether to give (6,7-dichloro-3-(1-(tetrahydro-2H-pyran-2-yl)-1H-pyrazol-4-yl)-1-(1-((trimethylsilyl)methyl)-1H-1,2,3-triazol-4-yl)-1H-indol-2-yl)methanol (220 mg, 98%) as a yellow solid. ES/MS (m/z): (³⁵Cl/³⁷Cl) 519.1/521.0 (M+H) and 501.1/503.1 (M+H—H₂O).

### Intermediate 414

### (6,7-Dichloro-1-(1-methyl-1H-1,2,3-triazol-4-yl)-3-(1-(tetrahydro-2H-pyran-2-yl)-1H-pyrazol-4-yl)-1H-indol-2-yl)methanol

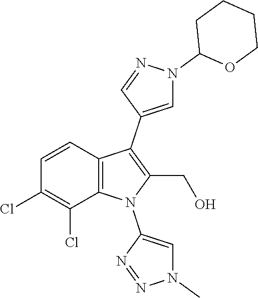 [SMILES: Cn1cc(-n2c(CO)c(-c3cnn(C4CCCCO4)c3)c3ccc(Cl)c(Cl)c32)nn1]

Dissolve (6,7-dichloro-3-(1-(tetrahydro-2H-pyran-2-yl)-1H-pyrazol-4-yl)-1-(1-((trimethylsilyl)methyl)-1H-1,2,3-triazol-4-yl)-1H-indol-2-yl)methanol (220 mg, 0.407 mmol) in THF (3 mL) and add TBAF (4 mL, 1M in THF). Stir at ambient temperature for 2 hrs. Dilute with EtOAc, wash with saturated aqueous NH₄Cl (3×), dry over anhydrous Na₂SO₄, filter, and concentrate under vacuum. Purify by flash column chromatography eluting with EtOAc in petroleum ether to give (6,7-dichloro-1-(1-methyl-1H-1,2,3-triazol-4-yl)-3-(1-(tetrahydro-2H-pyran-2-yl)-1H-pyrazol-4-yl)-1H-indol-2-yl)methanol (200 mg, 99%) as a yellow solid. ES/MS (m/z): (³⁵Cl/³⁷Cl) 447.1/449.0 (M+H), 429.1/430.9 (M+H—H₂O), and 345.1/347.0 (M+H-THP-H₂O).

### EXAMPLE 166

### [6,7-Dichloro-1-(1-methyltriazol -4-yl)-3-(1H-pyrazol-4-yl)indol -2-yl]methanol

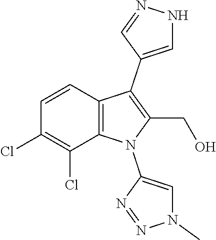 [SMILES: Cn1cc(-n2c(CO)c(-c3cn[nH]c3)c3ccc(Cl)c(Cl)c32)nn1]

Dissolve (6,7-dichloro-1-(1-methyl-1H-1,2,3-triazol-4-yl)-3-(1-(tetrahydro-2H-pyran-2-yl)-1H-pyrazol-4-yl)-1H-indol-2-yl)methanol (200 mg, 0.402 mmol) in THF (2 mL) and add 6M aqueous HCl (5 mL). Stir at ambient temperature for 2 hrs. Basify with saturated aqueous NaHCO₃ to pH=7, dilute with water, extract with EtOAc, wash with saturated aqueous NaCl, dry over with anhydrous Na₂SO₄, filter, and concentrate under vacuum. Purify by prep-HPLC to give [6,7-dichloro-1-(1-methyltriazol-4-yl)-3-(1H-pyrazol-4-yl)indol-2-yl]methanol (77.55 mg, 51%) as a white solid. ES/MS (m/z): (³⁵Cl/³⁷Cl) 363.0/364.9 (M+H).

### EXAMPLE 167

### (R)—N-((6,7-Dichloro-3-(1H-pyrazol-4-yl)-1H-indol-2-yl)methyl)-2-hydroxypropanamide

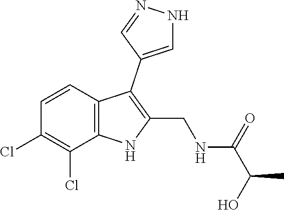 [SMILES: C[C@@H](O)C(=O)NCc1[nH]c2c(Cl)c(Cl)ccc2c1-c1cn[nH]c1]

Dissolve (R)-(+)-2-acetoxypropionic acid (115 mg, 0.853 mmol) in DMF (3 mL) and add HATU (412 mg, 1.06 mmol) and stir at ambient temperature 2 hrs. Add [6,7-dichloro-3-(1H-pyrazol-4-yl)-1H-indol-2-yl]methanamine (150 mg, 0.425 mmol, HCl salt) and DIPEA (0.22 mL, 1.30 mmol), then stir at ambient temperature for 16 hrs. Quench with water, extract with EtOAc, wash with saturated aqueous NaCl, dry over anhydrous sodium sulfate, filter, and concentrate under vacuum. Dissolve the residue in MeOH (2 mL) and add potassium carbonate (0.590 g, 4.30 mmol), then stir at ambient temperature for 2 hrs. Dilute with water, extract with EtOAc, wash with saturated aqueous NaCl, and dry over anhydrous sodium sulfate. Concentrate under vacuum and purify by flash column chromatography eluting with EtOAc in petroleum ether to give (R)—N-((6,7-dichloro-3-(1H-pyrazol-4-yl)-1H-indol-2-yl)methyl)-2-hydroxypropanamide (67.90 mg, 45%) as a white solid. ES/MS (m/z): (³⁵Cl/³⁷Cl) 353.0, 354.9 (M+H).

### EXAMPLE 168

### (S)—N-((6, 7-Dichloro-3-(1H-pyrazol-4-yl)-1H-indol-2-yl)methyl)-2-hydroxypropanamide

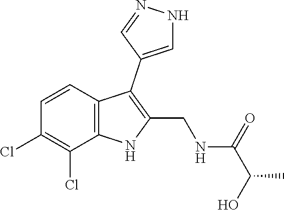 [SMILES: C[C@H](O)C(=O)NCc1[nH]c2c(Cl)c(Cl)ccc2c1-c1cn[nH]c1]

Dissolve [6,7-dichloro-3-(1H-pyrazol-4-yl)-1H-indol-2-yl]methanamine (150 mg, 0.425 mmol, HCl salt) and TEA (0.30 mL, 2.15 mmol) in DMF (3 mL). Add (S)-(−)-2-acetoxypropionyl chloride (0.082 mL, 0.635 mmol) at 0° C. and stir at ambient temperature for 2 hrs. Quench with water, extract with DCM, wash with saturated aqueous NaCl, dry over anhydrous sodium sulfate, filter, and concentrate under vacuum. Dissolve the residue in MeOH (2 mL) and add potassium carbonate (0.600 g, 4.34 mmol), then stir at ambient temperature for 2 hrs. Dilute with water, extract with EtOAc, wash with saturated aqueous NaCl, dry over anhydrous sodium sulfate, and concentrate under vacuum. Purify by flash column chromatography (EtOAc/PE) to give (S)—N-((6,7-dichloro-3-(1H-pyrazol-4-yl)-1H-indol-2-yl)methyl)-2-hydroxypropanamide (43.36 mg, 28%, ee=100%) as a white solid. ES/MS (m/z): (³⁵Cl/³⁷Cl) 353.0/354.9 (M+H).

### EXAMPLE 169

### N-(6,7-Dichloro-3-(1H-pyrazol-4-yl)-1H-indol-4-yl)-2,2-difluoro-3-hydroxypropanamide

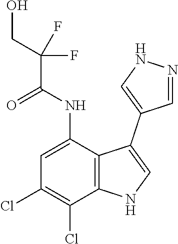 [SMILES: O=C(Nc1cc(Cl)c(Cl)c2[nH]cc(-c3cn[nH]c3)c12)C(F)(F)CO]

Dissolve 6,7-dichloro-3-(1H-pyrazol-4-yl)-1H-indol-4-amine (150 mg, 0.505 mmol) and ethyl 2,2-difluoro-3-hydroxypropanoate (330 mg, 2.06 mmol) in DCM (10 mL). Add Me₃Al (1.3 mL, 2.6 mmol, 2M in toluene) at 0° C. and stir at ambient temperature for 16-18 hrs. Quench with saturated aqueous sodium potassium tartrate and saturated aqueous sodium bicarbonate, extract with EtOAc, wash with saturated aqueous NaCl, dry over anhydrous Na₂SO₄, filter, and concentrate under vacuum. Purify by prep-HPLC to give N-(6,7-dichloro-3-(1H-pyrazol-4-yl)-1H-indol-4-yl)-2,2-difluoro-3-hydroxypropanamide (35.1 mg, 18%) as a white solid. ES/MS (m/z): (³⁵Cl/³⁷Cl) 375.2/377.2 (M+H).

### EXAMPLE 170

### N-(6,7-Dichloro-3-(1H-pyrazol-4-yl)-1H-indol-4-yl)-2-fluoroacetamide

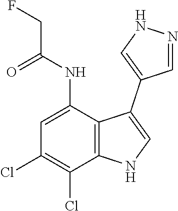 [SMILES: O=C(CF)Nc1cc(Cl)c(Cl)c2[nH]cc(-c3cn[nH]c3)c12]

Dissolve 6,7-dichloro-3-(1H-pyrazol-4-yl)-1H-indol-4-amine (60 mg, 0.22 mmol) and ethyl 2-fluoroacetate (100 mg, 0.943 mmol) in DCM (5 mL). Add Me₃Al (0.5 mL, 1.0 mmol, 2M in toluene) at 0° C. and stir at ambient temperature for 16-18 hrs. Quench with saturated aqueous sodium potassium tartrate and saturated aqueous sodium bicarbonate, extract with EtOAc, wash with saturated aqueous NaCl, dry over anhydrous Na₂SO₄, filter, and concentrate under vacuum. Purify by prep-HPLC to give N-(6,7-dichloro-3-(1H-pyrazol-4-yl)-1H-indol-4-yl)-2-fluoroacetamide (16.9 mg, 23%) as a white solid. ES/MS (m/z): (³⁵Cl/³⁷Cl) 327.2/329.2 (M+H).

### EXAMPLE 171

### N-[6, 7-Dichloro-3-(1H-pyrazol-4-yl)-1H-indol-4-yl]-2,2-difluoro-acetamide

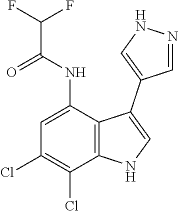 [SMILES: O=C(Nc1cc(Cl)c(Cl)c2[nH]cc(-c3cn[nH]c3)c12)C(F)F]

Dissolve 6,7-dichloro-3-(1H-pyrazol-4-yl)-1H-indol-4-amine (900 mg, 2.86 mmol) and DIPEA (1.5 mL, 8.60 mmol) in DCM (100 mL). Add difluoroacetic anhydride (1.50 g, 8.40 mmol) at 0° C. and stir at ambient temperature for 16-18 hrs. Concentrate under vacuum and purify by prep-HPLC to give N-[6,7-dichloro-3-(1H-pyrazol-4-yl)-1H-indol-4-yl]-2,2-difluoro-acetamide (473 mg, 48%) as a white solid. ES/MS (m/z): (³⁵Cl/³⁷Cl) 345.2/347.2 (M+H).

### Intermediate 415

### tert-Butyl N-[6-chloro-7-fluoro-1-(p-tolylsulfonyl)indol-4-yl]carbamate

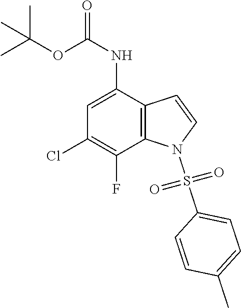 [SMILES: Cc1ccc(S(=O)(=O)n2ccc3c(NC(=O)OC(C)(C)C)cc(Cl)c(F)c32)cc1]

Dissolve 4-bromo-6-chloro-7-fluoro-1-(p-tolylsulfonyl)indole (10.0 g, 22.3 mmol), tert-butyl carbamate (5.8 g, 50 mmol), Pd₂(dba)₃ (2.00 g, 2.18 mmol), Xantphos (2.6 g, 4.5 mmol) and Cs₂CO₃ (22.0 g, 67.5 mmol) in 1,4-dioxane (150 mL). Purge with and stir at 100° C. for 3 hrs under N₂ atmosphere. Filter and dilute the filtrate with water, extract with EtOAc, dry over anhydrous Na₂SO₄, filter, and concentrate under vacuum. Purify by flash column chromatography eluting with EtOAc in petroleum ether to give tert-butyl N-[6-chloro-7-fluoro-1-(p-tolylsulfonyl)indol-4-yl]carbamate (12 g, 73%) as a yellow solid.

### Intermediate 416

### tert-Butyl N-(6-chloro-7-fluoro-1H-indol-4-yl)carbamate

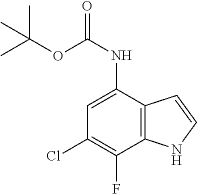 [SMILES: CC(C)(C)OC(=O)Nc1cc(Cl)c(F)c2[nH]ccc12]

Dissolve tert-butyl N-[6-chloro-7-fluoro-1-(p-tolylsulfonyl)indol-4-yl]carbamate (6.00 g, 8.20 mmol) in THF (10 mL) and add TBAF (80 mL, 1.0M in THF). Stir at ambient temperature for 16-18 hrs. Dilute with EtOAc, wash with saturated aqueous NH₄Cl (5×), dry over anhydrous Na₂SO₄, filter, and concentrate under vacuum to give tert-butyl N-(6-chloro-7-fluoro-1H-indol-4-yl)carbamate (3.5 g, 93%) as a yellow solid. ES/MS (m/z): (³⁵Cl/³⁷Cl) 229.1/231.1 (M+H-tBu).

### Intermediate 417

### tert-Butyl (6-chloro-7-fluoro-3-iodo-1H-indol-4-yl)carbamate

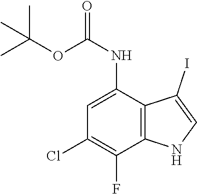 [SMILES: CC(C)(C)OC(=O)Nc1cc(Cl)c(F)c2[nH]cc(I)c12]

Dissolve tert-butyl N-(6-chloro-7-fluoro-1H-indol-4-yl)carbamate (3.5 g, 7.6 mmol) in DMF (40 mL) and add NIS (1.90 g, 8.30 mmol). Stir at 0° C. for 2 hrs. Dilute with EtOAc, wash with saturated aqueous sodium sulfite and saturated aqueous NaCl, dry over anhydrous Na₂SO₄, filter, and concentrate under vacuum. Purify by flash column chromatography eluting with EtOAc in petroleum ether to give tert-butyl (6-chloro-7-fluoro-3-iodo-1H-indol-4-yl)carbamate (2.20 g, 63%) as a white solid.

### Intermediate 418

### tert-Butyl N-[6-chloro-7-fluoro-3-(1-tetrahydropyran-2-ylpyrazol-4-yl)-1H-indol-4-yl]carbamate

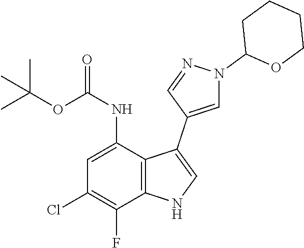 [SMILES: CC(C)(C)OC(=O)Nc1cc(Cl)c(F)c2[nH]cc(-c3cnn(C4CCCCO4)c3)c12]

Suspend tert-butyl (6-chloro-7-fluoro-3-iodo-1H-indol-4-yl)carbamate (2.2 g, 4.8 mmol), 1-(tetrahydro-2H-pyran-2-yl)-4-(4,4,5,5-tetramethyl-1,3,2-dioxaborolan-2-yl)-1H-pyrazole (2.7 g, 9.5 mmol), Pd(dtbpf)Cl₂ (650 mg, 0.977 mmol) and Na₂CO₃ (1.5 g, 14 mmol) in 1,4-dioxane (40 mL) and water (10 mL). Purge with N₂ and stir at 90° C. for 30 min under N₂ atmosphere. Filter and concentrate the filtrate under vacuum. Purify by column chromatography eluting with EtOAc in petroleum ether to give tert-butyl N-[6-chloro-7-fluoro-3-(1-tetrahydropyran-2-ylpyrazol-4-yl)-1H-indol-4-yl]carbamate (1.00 g, 43%) as a yellow solid. ES/MS (m/z): 435.1/437.1 (M+H) and 295.0/297.0 (M+H-tBu-THP).

### Intermediate 419

### tert-Butyl N-[6-chloro-7-fluoro-3-(1-tetrahydropyran-2-ylpyrazol-4-yl)-1-(2-triisopropylsilylethynyl)indol-4-yl]carbamate

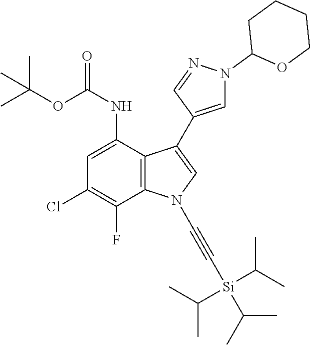 [SMILES: CC(C)[Si](C#Cn1cc(-c2cnn(C3CCCCO3)c2)c2c(NC(=O)OC(C)(C)C)cc(Cl)c(F)c21)(C(C)C)C(C)C]

Dissolve tert-butyl N-[6-chloro-7-fluoro-3-(1-tetrahydropyran-2-ylpyrazol-4-yl)-1H-indol-4-yl]carbamate (1.00 g, 2.07 mmol) in 1,4-dioxane (50 mL) and add (bromoethynyl)triisopropylsilane (2.8 g, 10 mmol), PEG-400 (400 mg), CuI (40 mg, 0.21 mmol) and Cs₂CO₃ (800 mg, 2.45 mmol). Purge with N₂ and stir at 125° C. for 16-18 hrs. Dilute with water, extract with EtOAc and wash with saturated aqueous NaCl, dry over Na₂SO₄, filter, and concentrate under vacuum. Purify by flash column chromatography eluting with EtOAc in petroleum ether to give tert-butyl N-[6-chloro-7-fluoro-3-(1-tetrahydropyran-2-ylpyrazol-4-yl)-1-(2-triisopropylsilylethynyl)indol-4-yl]carbamate (400 mg, 29%) as a yellow oil.

### Intermediate 420

### tert-Butyl N-[6-chloro-1-ethynyl-7-fluoro-3-(1-tetrahydropyran-2-ylpyrazol-4-yl)indol -4-yl]carbamate

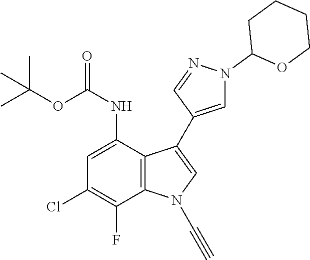 [SMILES: C#Cn1cc(-c2cnn(C3CCCCO3)c2)c2c(NC(=O)OC(C)(C)C)cc(Cl)c(F)c21]

Dissolve tert-butyl N-[6-chloro-7-fluoro-3-(1-tetrahydropyran-2-ylpyrazol-4-yl)-1-(2-triisopropylsilylethynyl)indol-4-yl]carbamate (700 mg, 1.14 mmol) in THF (6 mL) and add TBAF (3 mL, 1.0M in THF). Stir at ambient temperature for 2 hrs. Dilute with EtOAc, wash with saturated aqueous NH₄Cl (3×) and saturated aqueous NaCl, dry over Na₂SO₄, filter, and concentrate under vacuum. Purify by flash column chromatography eluting with EtOAc in petroleum ether to give tert-butyl N-[6-chloro-1-ethynyl-7-fluoro-3-(1-tetrahydropyran-2-ylpyrazol-4-yl)indol-4-yl]carbamate (400 mg, 74%) as a white solid. ES/MS (m/z): 459.2/461.1 (M+H).

### Intermediate 421

### tert-Butyl N-[1-[1-[2-[tert-butyl(dimethyl)silyl]oxyethyl]triazol-4-yl]-6-chloro-7-fluoro-3-(1-tetrahydropyran-2-ylpyrazol-4-yl)indol-4-yl]carbamate

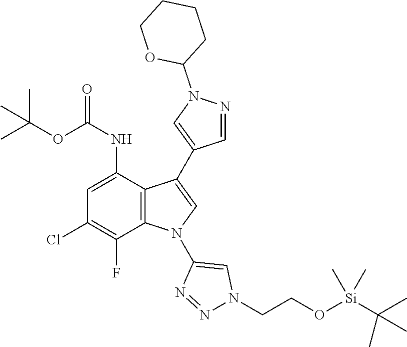 [SMILES: CC(C)(C)OC(=O)Nc1cc(Cl)c(F)c2c1c(-c1cnn(C3CCCCO3)c1)cn2-c1cn(CCO[Si](C)(C)C(C)(C)C)nn1]

Dissolve tert-butyl N-[6-chloro-1-ethynyl-7-fluoro-3-(1-tetrahydropyran-2-ylpyrazol-4-yl)indol-4-yl]carbamate (400 mg, 0.854 mmol) in DMF (8 mL) and water (8 mL) and add copper (12 mg, 0.19 mmol), cupric sulfate (28 mg, 0.18 mmol) and 2-azidoethoxy-tert-butyl-dimethyl-silane (230 mg, 1.09 mmol). Purge with N₂ and stir at 110° C. for 30 min under N₂ atmosphere. Dilute with water and extract with EtOAc (2×), dry the combined organic layers over anhydrous Na₂SO₄, filter, and concentrate under vacuum. Purify by flash column chromatography eluting with EtOAc in petroleum ether to give tert-butyl N-[1-[1[-2-[tert-butyl(dimethyl)silyl]oxyethyl]triazol-4-yl]-6-chloro-7-fluoro-3-(1-tetrahydropyran-2-ylpyrazol-4-yl)indol-4-yl]carbamate (300 mg, 50%) as a yellow oil. ES/MS (m/z): 660.3/662.3 (M+H), 576.2/578.3 (M+H-THP), and 520.2/522.1 (M+H-tBu-THP).

### Intermediate 422

### 2-[4-[4-Amino-6-chloro-7-fluoro-3-(1H-pyrazol-4-yl)indol-1-yl]triazol-1-yl]ethanol hydrochloride

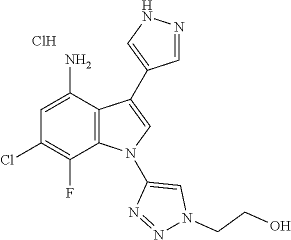 [SMILES: Nc1cc(Cl)c(F)c2c1c(-c1cn[nH]c1)cn2-c1cn(CCO)nn1 ; Cl]

Dissolve tert-butyl N-[1-[1-[2-[tert-butyl(dimethyl)silyl]oxyethyl]triazol-4-yl]-6-chloro-7-fluoro-3-(1-tetrahydropyran-2-ylpyrazol-4-yl)indol-4-yl]carbamate (300 mg, 0.427 mmol) in 4M HCl in MeOH (5 mL). Stir at ambient temperature for 2 hrs. Concentrate under vacuum to give 2-[4-[4-amino-6-chloro-7-fluoro-3-(1H-pyrazol-4-yl)indol-1-yl]triazol-1-yl]ethanol hydrochloride (220 mg, 0.541 mmol, 99+%) as a yellow solid. ES/MS (m/z): 362.1/364.1 (M+H).

### EXAMPLE 172

### N-[6-Chloro-7-fluoro-1-[1-(2-hydroxy ethyl)triazol -4-yl]-3-(1H-pyrazol-4-yl)indol -4-yl]-2,2-difluoro-acetamide

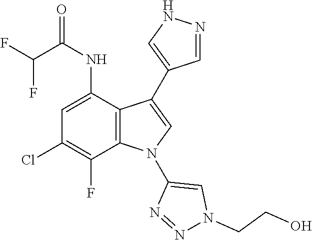 [SMILES: O=C(Nc1cc(Cl)c(F)c2c1c(-c1cn[nH]c1)cn2-c1cn(CCO)nn1)C(F)F]

Dissolve 2-[4-[4-amino-6-chloro-7-fluoro-3-(1H-pyrazol-4-yl)indol-1-yl]triazol-1-yl]ethanol (90 mg, 0.22 mmol, HCl salt), DIPEA (0.4 mL, 2.0 mmol) and DMAP (14 mg, 0.12 mmol) in DCM (3 mL). Add difluoroacetic anhydride (0.3 mL, 2.0 mmol) at 0° C. and stir at ambient temperature for 3 hrs. Combine with another batch run at 0.22×scale for work up and purification. Quench with saturated aqueous NaHCO₃, extract with DCM, wash with saturated aqueous NaCl, dry over anhydrous Na₂SO₄, filter, and concentrate under vacuum. Dissolve the residue in MeOH (3 mL) and add potassium carbonate (310 mg, 2.24 mmol). Stir at ambient temperature for 2 hrs. Dilute with water and extract with EtOAc, dry over anhydrous Na₂SO₄, filter, and concentrate under vacuum. Purify by prep-HPLC to give N-[6-chloro-7-fluoro-1-[1-(2-hydroxyethyl)triazol-4-yl]-3-(1H-pyrazol-4-yl)indol-4-yl]-2,2-difluoro-acetamide (54.7 mg, 46%) as a white solid. ES/MS (m/z): 440.0/442.0 (M+H).

### Intermediate 423

### 2-[4-[4-[3-[tert-Butyl(dimethyl)silyl]oxypropylamino]-6-chloro-7-fluoro-3-(1H-pyrazol-4-yl)indol-1-yl]triazol-1-yl]ethanol

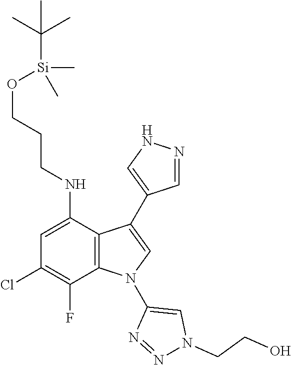 [SMILES: CC(C)(C)[Si](C)(C)OCCCNc1cc(Cl)c(F)c2c1c(-c1cn[nH]c1)cn2-c1cn(CCO)nn1]

Dissolve 2-[4-[4-amino-6-chloro-7-fluoro-3-(1H-pyrazol-4-yl)indol-1-yl]triazol-1-yl]ethanol hydrochloride (90 mg, 0.22 mmol) in MeOH (3 mL). Adjust to pH=7 with DIPEA (90 mg, 0.70 mmol) and stir at ambient temperature for 0.5 hr. Add acetic acid (15 mg, 0.25 mmol) to adjust to pH=5 and stir at ambient temperature for 0.5 hr. Add 3-[(tert-butyldimethylsilyl)oxy]-1-propanal (140 mg, 0.706 mmol) and stir at ambient temperature for 0.5 hr. Follow by adding sodium cyanoborohydride (45 mg, 0.68 mmol) and stir at ambient temperature for 3 hrs. Combine with another batch run at 0.22×scale for work up and purification. Dilute with water and adjust pH=8 with saturated aqueous NaHCO₃, extract with EtOAc, wash with saturated aqueous NaCl, dry over Na₂SO₄, filter, and concentrate under vacuum to give 2-[4-[4-[3-[tert-butyl(dimethyl)silyl]oxypropylamino]-6-chloro-7-fluoro-3-(1H-pyrazol-4-yl)indol-1-yl]triazol-1-yl]ethanol (120 mg, 83%) as a yellow oil.

### EXAMPLE 173

### 3-[[6-Chloro-7-fluoro-1-[1-(2-hydroxy ethyl)triazol-4-yl]-3-(1H-pyrazol-4-yl)indol-4-yl]amino]propan-1-ol

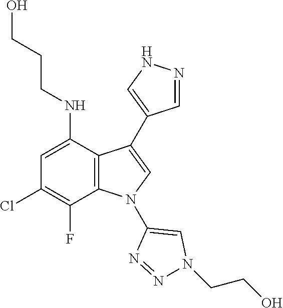 [SMILES: OCCCNc1cc(Cl)c(F)c2c1c(-c1cn[nH]c1)cn2-c1cn(CCO)nn1]

Dissolve 2-[4-[4-[3-[tert-butyl(dimethyl)silyl]oxypropylamino]-6-chloro-7-fluoro-3-(1H-pyrazol-4-yl)indol-1-yl]triazol-1-yl]ethanol (120 mg, 0.213 mmol) in THF (6 mL) and add 6M aqueous HCl (3 mL). Stir at ambient temperature for 2 hrs. Concentrate under vacuum and purify by prep-HPLC to give 3-[[6-chloro-7-fluoro-1-[1-(2-hydroxyethyl)triazol-4-yl]-3-(1H-pyrazol-4-yl)indol-4-yl]amino]propan-1-ol (15.8 mg, 17%) as a white solid. ES/MS (m/z): 420.1/422.0 (M+H).

### EXAMPLE 175

### 3-[[6,7-Dichloro-3-(1H-pyrazol-4-yl)-1H-indol-4-yl]amino]propane-1,2-diol

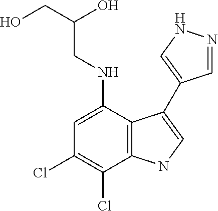 [SMILES: OCC(O)CNc1cc(Cl)c(Cl)c2[nH]cc(-c3cn[nH]c3)c12]

Suspend 6,7-dichloro-3-(1H-pyrazol-4-yl)-1H-indol-4-amine (80 mg, 0.27 mmol) in MeOH (5 mL). Add acetic acid (16 mg, 0.27 mmol) to adjust to pH=5 and stir at ambient temperature for 0.5 hr. Add glyceraldehyde (74 mg, 0.81 mmol) followed by sodium cyanoborohydride (53 mg, 0.80 mmol) and stir at ambient temperature for 16-18 hrs. Concentrate under vacuum and dilute with EtOAc, wash with saturated aqueous NaHCO₃ and saturated aqueous NaCl, dry over anhydrous sodium sulfate, filter, and concentrate under vacuum. Purify by prep-HPLC to give 3-[[6,7-dichloro-3-(1H-pyrazol-4-yl)-1H-indol-4-yl]amino]propane-1,2-diol (50.54 mg, 55%) as a gray solid. ES/MS (m/z): (³⁵Cl/³⁷Cl) 341.3/343.2 (M+H).

### EXAMPLE 176

### 2-Cyano-N-[6,7-dichloro-3-(1H-pyrazol-4-yl)-1H-indol-4-yl]acetamide

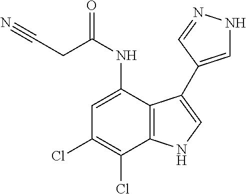 [SMILES: N#CCC(=O)Nc1cc(Cl)c(Cl)c2[nH]cc(-c3cn[nH]c3)c12]

Dissolve 6,7-dichloro-3-(1H-pyrazol-4-yl)-1H-indol-4-amine (40 mg, 0.11 mmol), cyanoacetic acid (18 mg, 0.21 mmol) and DIPEA (0.10 mL, 0.60 mmol) in DMF (3 mL) and add HATU (81 mg, 0.21 mmol). Stir at ambient temperature for 2 hrs. Purify by prep-HPLC to give 2-cyano-N-[6,7-dichloro-3-(1H-pyrazol-4-yl)-1H-indol-4-yl]acetamide (5.08 mg, 14%) as a white solid. ES/MS (m/z): (³⁵Cl/³⁷Cl) 334.2/336.2 (M+H).

### EXAMPLE 177

### 6,7-Dichloro-N-(oxetan-3-yl)-3-(1H-pyrazol-4-yl)-1H-indol-4-amine

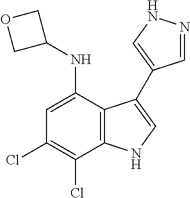 [SMILES: Clc1cc(NC2COC2)c2c(-c3cn[nH]c3)c[nH]c2c1Cl]

Suspend 6,7-dichloro-3-(1H-pyrazol-4-yl)-1H-indol-4-amine (50 mg, 0.18 mmol) in MeOH (3 mL). Add acetic acid (11 mg, 0.18 mmol) to adjust to pH=5 and stir at ambient temperature for 0.5 hr. Add 3-oxetanone (47 mg, 0.62 mmol) and stir at ambient temperature for 3 hrs. Add sodium cyanoborohydride (35 mg, 0.53 mmol) and stir at 60° C. for 16-18 hrs. Purify by prep-HPLC to give 6,7-dichloro-N-(oxetan-3-yl)-3-(1H-pyrazol-4-yl)-1H-indol-4-amine (32.0 mg, 56%) as a yellow solid. ES/MS (m/z): (³⁵Cl/³⁷Cl) 323.2/325.3 (M+H).

### Intermediate 430

### 2-[6,7-Dichloro-3-(1-tetrahydropyran-2-ylpyrazol -4-yl)-1H-indol-2-yl]-N-(1H-pyrazol -3-ylmethyl) acetamide

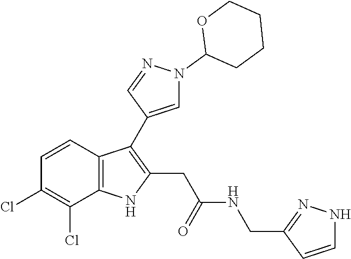 [SMILES: O=C(Cc1[nH]c2c(Cl)c(Cl)ccc2c1-c1cnn(C2CCCCO2)c1)NCc1cc[nH]n1]

Dissolve 2-[6,7-dichloro-3-(1-tetrahydropyran-2-ylpyrazol-4-yl)-1H-indol-2-yl] acetic acid (150 mg, 0.361 mmol), (1H-pyrazol-3-yl)methanamine (92 mg, 0.90 mmol), DIPEA (0.315 mL, 1.81 mmol) and HATU (280 mg, 0.722 mmol) in DMF (6 mL) and stir at ambient temperature for 2 hrs. Quench with water, extract with EtOAc, wash with saturated aqueous NaCl, dry over anhydrous Na₂SO₄, filter, and concentrate under vacuum to give 2-[6,7-dichloro-3-(1-tetrahydropyran-2-ylpyrazol-4-yl)-1H-indol-2-yl]-N-(1H-pyrazol-3-ylmethyl) acetamide (300 mg, 96%) as a yellow oil. ES/MS (m/z): (³⁵Cl/³⁷Cl) 473.0/475.0 (M+H) and 389.0/391.0 (M+H-THP).

### EXAMPLE 178

### 2-[6,7-Dichloro-3-(1H-pyrazol-4-yl)-1H-indol-2-yl]-N-(1H-pyrazol-3-ylmethyl) acetamide

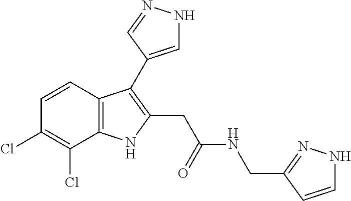 [SMILES: O=C(Cc1[nH]c2c(Cl)c(Cl)ccc2c1-c1cn[nH]c1)NCc1cc[nH]n1]

Dissolve 2-[6,7-dichloro-3-(1-tetrahydropyran-2-ylpyrazol-4-yl)-1H-indol-2-yl]-N-(1H-pyrazol-3-ylmethyl) acetamide (300 mg, 0.348 mmol) in DCM (3 mL) and add TFA (1.50 mL), then stir at ambient temperature for 2 hrs. Concentrate under vacuum and purify by reverse phase prep-HPLC to give 2-[6,7-dichloro-3-(1H-pyrazol-4-yl)-1H-indol-2-yl]-N-(1H-pyrazol-3-ylmethyl) acetamide (37.2 mg, 26%) as a white solid. ES/MS (m/z): (³⁵Cl/³⁷Cl) 389.1/391.1 (M+H).

### EXAMPLE 179

### 2-[2-[6,7-Dichloro-10-(1H-pyrazol-4-yl)-3,4-dihydro-1H-pyrazino[1,2-a]indol-2-yl]-2-oxo-ethoxy]-N-ethyl-acetamide

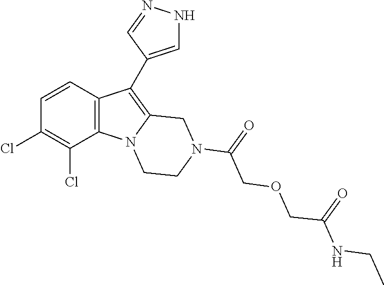 [SMILES: CCNC(=O)COCC(=O)N1CCn2c(c(-c3cn[nH]c3)c3ccc(Cl)c(Cl)c32)C1]

Dissolve 2-[(ethylcarbamoyl) methoxy] acetic acid (50 mg, 0.25 mmol), 6,7-dichloro-10-(1H-pyrazol-4-yl)-1,2,3,4-tetrahydropyrazino[1,2-a] indole (50 mg, 0.14 mmol, HCl salt), DIPEA (0.13 mL, 0.73 mmol) and HATU (112 mg, 0.289 mmol) in DMF (6 mL) and stir at ambient temperature for 2 hrs. Quench with water, extract with EtOAc, wash with saturated aqueous NaCl, dry over anhydrous Na₂SO₄, filter, and concentrate under vacuum. Purify by prep-HPLC to give 2-[2-[6,7-dichloro-10-(1H-pyrazol-4-yl)-3,4-dihydro-1H-pyrazino[1,2-a]indol-2-yl]-2-oxo-ethoxy]-N-ethyl-acetamide (12.3 mg, 20%) as a light-yellow oil. ES/MS (m/z): (³⁵Cl/³⁷Cl) 450.3/452.3 (M+H).

### EXAMPLE 180

### 5-[6,7-Dichloro-10-(1H-pyrazol-4-yl)-3,4-dihydro-1H-pyrazino[1,2-a]indole-2-carbonyl]oxazolidin-2-one

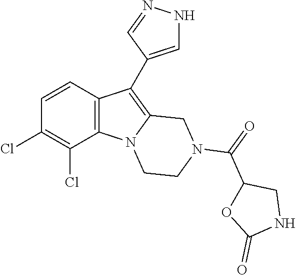 [SMILES: O=C1NCC(C(=O)N2CCn3c(c(-c4cn[nH]c4)c4ccc(Cl)c(Cl)c43)C2)O1]

Dissolve 2-oxooxazolidine-5-carboxylic acid (100 mg, 0.725 mmol) and 6,7-dichloro-10-(1H-pyrazol-4-yl)-1,2,3,4-tetrahydropyrazino[1,2-a]indole (100 mg, 0.277 mmol, HCl salt), DIPEA (0.48 mL, 2.8 mmol) and HATU (375 mg, 0.967 mmol) in DMF (3 mL) and stir at ambient temperature for 2 hrs. Quench with water, extract with EtOAc, wash with saturated aqueous NaCl, dry over anhydrous Na₂SO₄, filter, and concentrate under vacuum. Purify by prep-HPLC to give 5-[6,7-dichloro-10-(1H-pyrazol-4-yl)-3,4-dihydro-1H-pyrazino[1,2-a]indole-2-carbonyl]oxazolidin-2-one (43.3 mg, 37%) as a yellow solid. ES/MS (m/z): (³⁵Cl/³⁷Cl) 420.3/422.3 (M+H).

### EXAMPLE 181

### 2-[[6,7-Dichloro-2-[2-[(2-methyl-1,2,4-triazol-3-yl)methoxy]acetyl]-10-(1H-pyrazol-4-yl)-3,4,5a,9a-tetrahydro-1H-pyrazino[1,2-a]indol-9-yl]oxy]acetonitrile

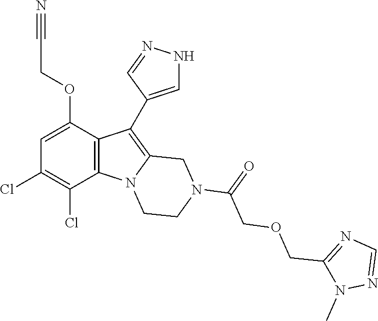 [SMILES: Cn1ncnc1COCC(=O)N1CCn2c(c(-c3cn[nH]c3)c3c(OCC#N)cc(Cl)c(Cl)c32)C1]

Dissolve 2-[[6,7-dichloro-10-(1H-pyrazol-4-yl)-1,2,3,4-tetrahydropyrazino[1,2-a]indol-9-yl]oxy] acetonitrile (40 mg, 0.099 mmol, HCl salt), 2-[(1-methyl-1H-1,2,4-triazol-5-yl)methoxy]acetic acid (34 mg, 0.14 mmol), DIPEA (0.18 mL, 1.0 mmol) and HATU (136 mg, 0.351 mmol) in DMF (3 mL) and stir at ambient temperature for 2 hrs. Quench with water, extract with EtOAc, wash with saturated aqueous NaCl, dry over anhydrous Na₂SO₄, filter, and concentrate under vacuum. Purify prep-HPLC to give 2-[[6,7-dichloro-2-[2-[(2-methyl-1,2,4-triazol-3-yl)methoxy]acetyl]-10-(1H-pyrazol-4-yl)-3,4,5a,9a-tetrahydro-1H-pyrazino[1,2-a]indol-9-yl]oxy]acetonitrile (4.7 mg, 9%) as a white solid. ES/MS (m/z): (³⁵Cl/³⁷Cl) 515.4/517.3 (M+H).

### Intermediate 431

### tert-Butyl N-[2-[6,7-dichloro-10-(1H-pyrazol-4-yl)-3,4-dihydro-1H-pyrazino[1,2-a]indol-2-yl]-2-oxo-ethyl]carbamate

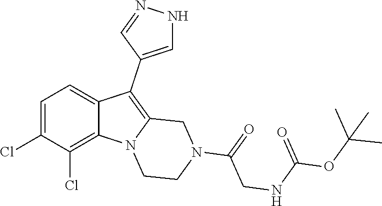 [SMILES: CC(C)(C)OC(=O)NCC(=O)N1CCn2c(c(-c3cn[nH]c3)c3ccc(Cl)c(Cl)c32)C1]

Dissolve 6,7-dichloro-10-(1H-pyrazol-4-yl)-1,2,3,4-tetrahydropyrazino[1,2-a]indole (100 mg, 0.291 mmol, HCl salt) and DIEA (150 mg, 1.16 mmol) in DMF (2 mL). Stir at ambient temperature for 10 minutes. Add (tert-butoxycarbonyl)glycine (80 mg, 0.46 mmol) and HATU (135 mg, 0.348 mmol), and stir for 1 hr. Dilute with water and extract the aqueous layer with EtOAc (2×). Wash the combined organic layers with saturated aqueous NaCl, dry over anhydrous sodium sulfate, filter, and concentrate under vacuum to give tert-butyl N-[2-[6,7-dichloro-10-(1H-pyrazol-4-yl)-3,4-dihydro-1H-pyrazino[1,2-a]indol-2-yl]-2-oxo-ethyl]carbamate (120 mg, 61%) as a yellow oil. ES/MS (m/z): (³⁵Cl/³⁷Cl) 408.0/409.8 (M+H-tBu) and 364.0/365.9 (M+H-Boc).

### Intermediate 432

### 2-Amino-1-[6,7-dichloro-10-(1H-pyrazol-4-yl)-3,4-dihydro-1H-pyrazino[1,2-a]indol-2-yl]ethanone hydrochloride

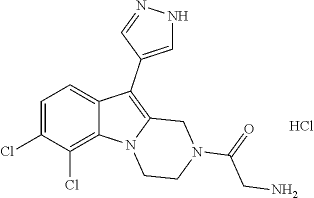 [SMILES: NCC(=O)N1CCn2c(c(-c3cn[nH]c3)c3ccc(Cl)c(Cl)c32)C1 ; Cl]

Dissolve tert-butyl N-[2-[6,7-dichloro-10-(1H-pyrazol-4-yl)-3,4-dihydro-1H-pyrazino[1,2-a]indol-2-yl]-2-oxo-ethyl]carbamate (120 mg, 0.173 mmol) in 4M HCl in MeOH (3 mL) and stir at ambient temperature for 1 hr. Concentrate under vacuum to give 2-amino-1-[6,7-dichloro-10-(1H-pyrazol-4-yl)-3,4-dihydro-1H-pyrazino[1,2-a]indol-2-yl]ethanone hydrochloride (83 mg, 90%) as a light-yellow solid. ES/MS (m/z): (³⁵Cl/³⁷Cl) 364.1/366.1 (M+H).

### EXAMPLE 182

### 3-[2-[6,7-Dichloro-10-(1H-pyrazol-4-yl)-3,4-dihydro-1H-pyrazino[1,2-a]indol-2-yl]-2-oxo-ethyl]-1,1-dimethyl-urea

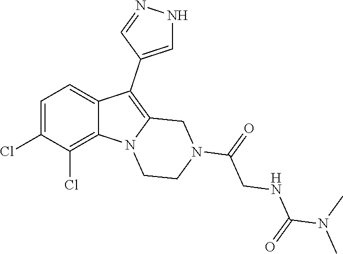 [SMILES: CN(C)C(=O)NCC(=O)N1CCn2c(c(-c3cn[nH]c3)c3ccc(Cl)c(Cl)c32)C1]

Dissolve 2-amino-1-[6,7-dichloro-10-(1H-pyrazol-4-yl)-3,4-dihydro-1H-pyrazino[1,2-a]indol-2-yl]ethanone (83 mg, 0.17 mmol, HCl salt) and TEA (0.10 mL, 0.72 mmol) in DCM (3 mL), then stir at 0° C. for 10 min. Add dimethylcarbamoyl chloride (0.045 mL, 0.49 mmol) at 0° C., stir at ambient temperature for 1 hr. Dilute with DCM and wash with saturated aqueous sodium bicarbonate. Extract the aqueous layer with DCM, wash the combined organic layers with saturated aqueous NaCl, dry over anhydrous sodium sulfate, filter, and concentrate under vacuum. Purify by prep-HPLC to give 3-[2-[6,7-dichloro-10-(1H-pyrazol-4-yl)-3,4-dihydro-1H-pyrazino[1,2-a]indol-2-yl]-2-oxo-ethyl]-1,1-dimethyl-urea (24.43 mg, 35%) as a white solid. ES/MS (m/z): (³⁵Cl/³⁷Cl) 435.3/437.3 (M+H).

### Intermediate 433

### 6, 7-Dichloro-4-(2-fluoroethoxy)-1-(p-tolylsulfonyl)indole

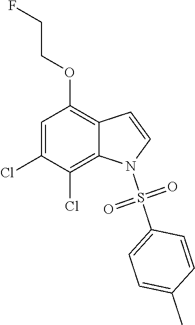 [SMILES: Cc1ccc(S(=O)(=O)n2ccc3c(OCCF)cc(Cl)c(Cl)c32)cc1]

Dissolve 6,7-dichloro-1-(p-tolylsulfonyl)indol-4-ol (500 mg, 1.26 mmol) in DMF (5 mL). Add 1-fluoro-2-iodoethane (0.23 mL, 2.6 mmol) and potassium carbonate (175 mg, 1.27 mmol), then stir at 60° C. for 2 hrs. Combine with another batch run at 0.10×scale for work up and purification. Cool to ambient temperature, dilute with EtOAc, and wash with water. Extract the aqueous layer with EtOAc (3×). Wash the combined organic layers with saturated aqueous NaCl, dry over sodium sulfate, filter, and concentrate under vacuum. Purify by flash column chromatography eluting with EtOAc in petroleum ether to give 6,7-dichloro-4-(2-fluoroethoxy)-1-(p-tolylsulfonyl)indole (637 mg, 95%) as a yellow solid. ES/MS (m/z): (³⁵Cl/³⁷Cl) 402.0/404.1 (M+H).

### Intermediate 434

### 6, 7-Dichloro-4-(2-fluoroethoxy)-1H-indole

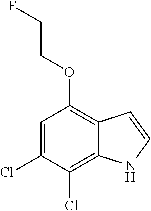 [SMILES: FCCOc1cc(Cl)c(Cl)c2[nH]ccc12]

Dissolve 6,7-dichloro-4-(2-fluoroethoxy)-1-(p-tolylsulfonyl)indole (637 mg, 1.31 mmol) in THF (5 mL), add TBAF (5 mL, 1.0M in THF), and stir at ambient temperature for 4 hrs. Dilute with EtOAc, wash with saturated NH₄Cl solution (3×), dry over anhydrous sodium sulfate, filter, and concentrate under vacuum to give 6,7-dichloro-4-(2-fluoroethoxy)-1H-indole (446 mg, 97%) as a brown oil. ES/MS (m/z): 248.1/250.2 (M+H).

### Intermediate 435

### 6,7-Dichloro-4-(2-fluoroethoxy)-3-iodo-1H-indole

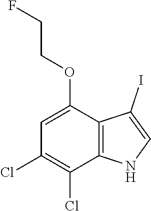 [SMILES: FCCOc1cc(Cl)c(Cl)c2[nH]cc(I)c12]

Dissolve 6,7-dichloro-4-(2-fluoroethoxy)-1H-indole (446 mg, 1.28 mmol) in DMF (5 mL). Add NIS (352 mg, 1.53 mmol) and stir at ambient temperature for 1.5 hr. Concentrate under vacuum, dilute with EtOAc, and wash with water. Extract the aqueous layer with EtOAc (3×). Wash the combined organic layers with saturated aqueous NaCl, dry over sodium sulfate, filter, and concentrate under vacuum. Purify by flash column chromatography eluting with EtOAc in petroleum ether to give 6,7-dichloro-4-(2-fluoroethoxy)-3-iodo-1H-indole (308 mg, 58%) as a purple solid.

### Intermediate 436

### 6,7-Dichloro-4-(2-fluoroethoxy)-3-(1-tetrahydropyran-2-ylpyrazol-4-yl)-1H-indole

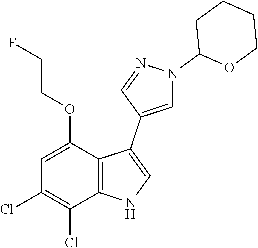 [SMILES: FCCOc1cc(Cl)c(Cl)c2[nH]cc(-c3cnn(C4CCCCO4)c3)c12]

Suspend 6,7-dichloro-4-(2-fluoroethoxy)-3-iodo-1H-indole (308 mg, 0.741 mmol), 1-(tetrahydro-2H-pyran-2-yl)-4-(4,4,5,5-tetramethyl-1,3,2-dioxaborolan-2-yl)-1H-pyrazole (326 mg, 1.11 mmol), Pd(dtbpf)Cl₂ (50 mg, 0.076 mmol) and sodium carbonate (236 mg, 2.23 mmol) in 1,4-dioxane (4 mL) and water (1 mL). Purge with N₂ (3×) and heat at 90° C. for 2 hrs. Cool to ambient temperature, dilute with water, extract with EtOAc, wash with saturated aqueous NaCl, dry over sodium sulfate, filter, and concentrate under vacuum. Purify by flash column chromatography eluting with EtOAc in petroleum ether to give 6,7-dichloro-4-(2-fluoroethoxy)-3-(1-tetrahydropyran-2-ylpyrazol-4-yl)-1H-indole (238 mg, 66%) as a yellow solid. ES/MS (m/z): 398.1/400.0 (M+H).

### EXAMPLE 183

### 6,7-Dichloro-4-(2-fluoroethoxy)-3-(1H-pyrazol-4-yl)-1H-indole

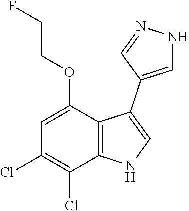 [SMILES: FCCOc1cc(Cl)c(Cl)c2[nH]cc(-c3cn[nH]c3)c12]

Suspend 6,7-dichloro-4-(2-fluoroethoxy)-3-(1-tetrahydropyran-2-ylpyrazol-4-yl)-1H-indole (233 mg, 0.480 mmol) in DCM (5 mL). Add TFA (5 mL) and stir at ambient temperature for 16-18 hrs. Concentrate under vacuum and purify by prep-HPLC to give 6,7-dichloro-4-(2-fluoroethoxy)-3-(1H-pyrazol-4-yl)-1H-indole (29.20 mg, 18%) as a yellow solid. ES/MS (m/z): (³⁵Cl/³⁷Cl) 314.2/316.2 (M+H).

### Intermediate 437

### 6,7-Dichloro-4-(2,2-di fluoroethoxy)-1-(p-tolylsulfonyl)indole

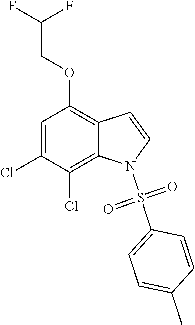 [SMILES: Cc1ccc(S(=O)(=O)n2ccc3c(OCC(F)F)cc(Cl)c(Cl)c32)cc1]

Dissolve 6,7-dichloro-1-(p-tolylsulfonyl) indol-4-ol (500 mg, 1.26 mmol) in DMF (3 mL). Add 1,1-difluoro-2-iodoethane (0.24 mL, 2.55 mmol) and potassium carbonate (175 mg, 1.27 mmol) and stir at 60° C. for for 16-18 hrs. Dilute with EtOAc, and wash with water. Extract the aqueous layer with EtOAc (3×). Wash the combined organic layers with saturated aqueous NaCl, dry over sodium sulfate, filter, and concentrate under vacuum. Purify by flash column chromatography eluting with EtOAc in petroleum ether to give 6,7-dichloro-4-(2,2-difluoroethoxy)-1-(p-tolylsulfonyl)indole (195 mg, 22%) as a white solid. ES/MS (m/z): 419.9/421.9 (M+H).

### Intermediate 438

### 6,7-Dichloro-4-(2,2-difluoroethoxy)-1H-indole

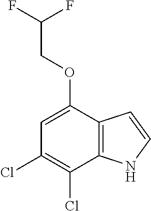 [SMILES: FC(F)COc1cc(Cl)c(Cl)c2[nH]ccc12]

Dissolve 6,7-dichloro-4-(2,2-difluoroethoxy)-1-(p-tolylsulfonyl)indole (195 mg, 0.274 mmol) in THF (2 mL), add TBAF (2 mL, 1.0M in THF), and stir at ambient temperature for 16-18 hrs. Dilute with EtOAc, wash with saturated aqueous NH₄Cl, dry over anhydrous sodium sulfate, filter, and concentrate under vacuum to give 6,7-dichloro-4-(2,2-difluoroethoxy)-1H-indole (180 mg, 99+%) as a yellow oil. ES/MS (m/z): 265.9/267.8 (M+H).

### Intermediate 439

### 6,7-Dichloro-4-(2,2-difluoroethoxy)-3-iodo-1H-indole

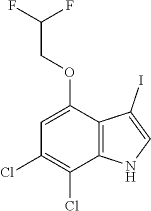 [SMILES: FC(F)COc1cc(Cl)c(Cl)c2[nH]cc(I)c12]

Dissolve 6,7-dichloro-4-(2,2-difluoroethoxy)-1H-indole (180 mg, 0.561 mmol) in DMF (3 mL). Add NIS (157 mg, 0.684 mmol) and stir at ambient temperature for 16-18 hrs. Concentrate under vacuum, dilute with EtOAc, and wash with water. Extract the aqueous layer with EtOAc (3×). Wash the combined organic layers with saturated aqueous NaCl, dry over sodium sulfate, filter, and concentrate under vacuum. Purify by flash column chromatography eluting with EtOAc in petroleum ether to give 6,7-dichloro-4-(2,2-difluoroethoxy)-3-iodo-1H-indole (213 mg, 91%) as a dark red solid.

### Intermediate 440

### 6,7-Dichloro-4-(2,2-difluoroethoxy)-3-(1-tetrahydropyran-2-ylpyrazol-4-yl)-1H-indole

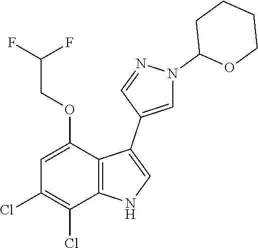 [SMILES: FC(F)COc1cc(Cl)c(Cl)c2[nH]cc(-c3cnn(C4CCCCO4)c3)c12]

Suspend 6,7-dichloro-4-(2,2-difluoroethoxy)-3-iodo-1H-indole (213 mg, 0.516 mmol), 1-(tetrahydro-2H-pyran-2-yl)-4-(4,4,5,5-tetramethyl-1,3,2-dioxaborolan-2-yl)-1H-pyrazole (227 mg, 0.775 mmol), Pd(dtbpf)Cl₂ (35 mg, 0.053 mmol) and sodium carbonate (165 mg, 1.56 mmol) in 1,4-dioxane (4 mL) and water (1 mL). Purge with N₂ and heat at 90° C. for 1 hr. Cool to ambient temperature, dilute with water, extract with EtOAc, wash with saturated aqueous NaCl, dry over sodium sulfate, filter, and concentrate under vacuum. Purify by flash column chromatography eluting with EtOAc in petroleum ether to give 6,7-dichloro-4-(2,2-difluoroethoxy)-3-(1-tetrahydropyran-2-ylpyrazol-4-yl)-1H-indole (51 mg, 21%) as a yellow solid. ES/MS (m/z): 416.1/418.1 (M+H).

### EXAMPLE 184

### 6,7-Dichloro-4-(2,2-difluoroethoxy)-3-(1H-pyrazol-4-yl)-1H-indole

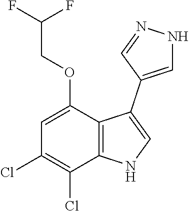 [SMILES: FC(F)COc1cc(Cl)c(Cl)c2[nH]cc(-c3cn[nH]c3)c12]

Dissolve 6,7-dichloro-4-(2,2-difluoroethoxy)-3-(1-tetrahydropyran-2-ylpyrazol-4-yl)-1H-indole (50 mg, 0.11 mmol) in DCM (3 mL). Add TFA (3 mL) and stir at ambient temperature for 2 hrs. Concentrate under vacuum and purify by prep-HPLC to give 6,7-dichloro-4-(2,2-difluoroethoxy)-3-(1H-pyrazol-4-yl)-1H-indole (18.67 mg, 52%) as a white solid. ES/MS (m/z): (³⁵Cl/³⁷Cl) 332.2/334.2 (M+H).

### Intermediate 441

### tert-Butyl 4-(4-amino-6,7-dichloro-1H-indol-3-yl)pyrazole-1-carboxylate)

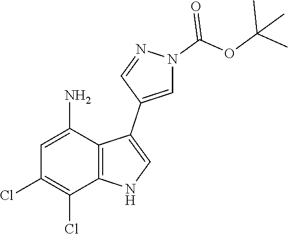 [SMILES: CC(C)(C)OC(=O)n1cc(-c2c[nH]c3c(Cl)c(Cl)cc(N)c23)cn1]

Suspend 6,7-dichloro-3-(1H-pyrazol-4-yl)-1H-indol-4-amine (200 mg, 0.674 mmol), TEA (0.7 mL, 5 mmol) in DCM (5 mL). Add Boc₂O (0.3 mL, 1.0 mmol) dropwise at 0° C. and stir at ambient temperature for 16-18 hrs. Dilute with water, extract with DCM, wash with saturated aqueous NaCl, dry over anhydrous sodium sulfate, filter, and concentrate under vacuum. Purify by flash column chromatography eluting with EtOAc in petroleum ether to give tert-butyl 4-(4-amino-6,7-dichloro-1H-indol-3-yl)pyrazole-1-carboxylate (220 mg, 62%) as a white solid. ES/MS (m/z): (³⁵Cl/³⁷Cl) 366.9/368.9 (M+H) and 307.8/309.8 (M+H-tBu).

### Intermediate 442

### tert-Butyl 4-[6, 7-dichloro-4-(prop -2-enoyl amino)-1H-indol-3-yl]pyrazole-1-carboxylate

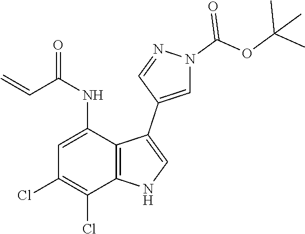 [SMILES: C=CC(=O)Nc1cc(Cl)c(Cl)c2[nH]cc(-c3cnn(C(=O)OC(C)(C)C)c3)c12]

Dissolve tert-butyl 4-(4-amino-6,7-dichloro-1H-indol-3-yl)pyrazole-1-carboxylate (220 mg, 0.419 mmol) and TEA (0.3 mL, 2.0 mmol) in DCM (5 mL) and add acryloyl chloride (46 mg, 0.51 mmol) at 0° C. Stir at 0° C. for 2 hrs. Dilute with water, extract with DCM, dry over anhydrous sodium sulfate, filter, and concentrate under vacuum. Purify by flash column chromatography eluting with MeOH in DCM to give tert-butyl 4-[6,7-dichloro-4-(prop-2-enoylamino)-1H-indol-3-yl]pyrazole-1-carboxylate (130 mg, 63%) as a yellow solid. ES/MS (m/z): (³⁵Cl/³⁷Cl) 320.9/322.9 (M+H-Boc).

### Intermediate 443

### tert-Butyl 4-[6,7-dichloro-4-(2,3-dihydroxypropanoyl amino)-1H-indol-3-yl]pyrazole-1-carboxylate

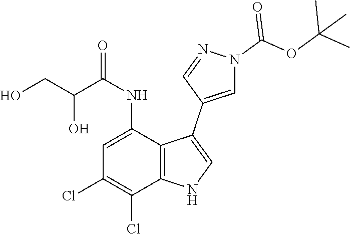 [SMILES: CC(C)(C)OC(=O)n1cc(-c2c[nH]c3c(Cl)c(Cl)cc(NC(=O)C(O)CO)c23)cn1]

Dissolve tert-butyl 4-[6,7-dichloro-4-(prop-2-enoylamino)-1H-indol-3-yl]pyrazole-1-carboxylate (170 mg, 0.383 mmol), NMO (55 mg, 0.46 mmol) in THF (4 mL), t-BuOH (1 mL) and water (0.5 mL). Add osmium tetroxide (50 mg, 0.20 mmol) and stir at ambient temperature for 3 hrs. Quench by the addition 5% aqueous Na₂SO₃, stir for 15 min, and pour into water. Extract with EtOAc, dry over sodium sulfate, filter, and concentrate under vacuum. Purify by flash column chromatography eluting with MeOH in DCM to give tert-butyl 4-[6,7-dichloro-4-(2,3-dihydroxypropanoylamino)-1H-indol-3-yl]pyrazole-1-carboxylate (140 mg, 67%) as a colorless solid. ES/MS (m/z): (³⁵Cl/³⁷Cl) 455.0/456.9 (M+H) and 354.9/356.9 (M+H-Boc).

### EXAMPLE 185

### N-[6,7-Dichloro-3-(1H-pyrazol-4-yl)-1H-indol-4-yl]-2,3-dihydroxy-propanamide

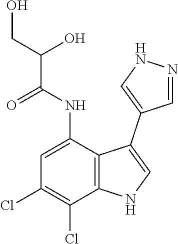 [SMILES: O=C(Nc1cc(Cl)c(Cl)c2[nH]cc(-c3cn[nH]c3)c12)C(O)CO]

Suspend tert-butyl 4-[6,7-dichloro-4-(2,3-dihydroxypropanoylamino)-1H-indol-3-yl]pyrazole-1-carboxylate (140 mg, 0.258 mmol) in 4M HCl in dioxane (5 mL) and stir at ambient temperature for 2 hrs. Concentrate under vacuum and purify by prep-HPLC to give N-[6,7-dichloro-3-(1H-pyrazol-4-yl)-1H-indol-4-yl]-2,3-dihydroxy-propanamide (10.16 mg, 11%) as an off-white solid. ES/MS (m/z): (³⁵Cl/³⁷Cl) 355.2/357.2 (M+H).

### Intermediate 444

### N-[3-[tert-Butyl(dimethyl)silyl]oxy-2,2-difluoro-propyl]-6,7-dichloro-3-(1-tetrahydropyran-2-ylpyrazol-4-yl)-1H-indol-4-amine

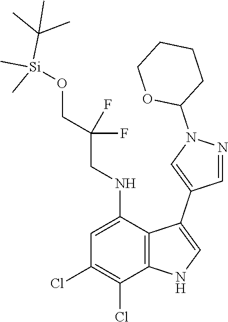 [SMILES: CC(C)(C)[Si](C)(C)OCC(F)(F)CNc1cc(Cl)c(Cl)c2[nH]cc(-c3cnn(C4CCCCO4)c3)c12]

Suspend 4-bromo-6,7-dichloro-1-(p-tolylsulfonyl)-3-(1-tetrahydropyran-2-ylpyrazol-4-yl)indole (200 mg, 0.334 mmol), 3-[tert-butyl(dimethyl)silyl]oxy-2,2-difluoro-propan-1-amine (CAS: 2416479-54-2, 130 mg, 0.519 mmol), t-BuONa (102 mg, 1.03 mmol) and (t-Bu₃P)₂Pd (30 mg, 0.058 mmol) in 1,4-dioxane (5 mL). Purge with N₂, then stir at 100° C. for 16-18 hrs. Cool to ambient temperature, dilute with water, extract with EtOAc (2×), wash the combined organic layers with saturated aqueous NaCl (2×), dry over anhydrous Na₂SO₄, filter, and concentrate under vacuum. Purify by flash column chromatography eluting with EtOAc in petroleum ether to give N-[3-[tert-butyl(dimethyl)silyl]oxy-2,2-difluoro-propyl]-6,7-dichloro-3-(1-tetrahydropyran-2-ylpyrazol-4-yl)-1H-indol-4-amine (97 mg, 48%) as a brown oil. ES/MS (m/z): (³⁵Cl/³⁷Cl) 559.0/561.1 (M+H) and 475.0/477.0 (M+H-THP).

### EXAMPLE 186

### 3-[[6,7-Dichloro-3-(1H-pyrazol-4-yl)-1H-indol-4-yl]amino]-2,2-difluoro-propan-1-ol

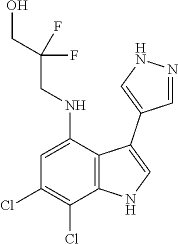 [SMILES: OCC(F)(F)CNc1cc(Cl)c(Cl)c2[nH]cc(-c3cn[nH]c3)c12]

Dissolve N-[3-[tert-butyl(dimethyl)silyl]oxy-2,2-difluoro-propyl]-6,7-dichloro-3-(1-tetrahydropyran-2-ylpyrazol-4-yl)-1H-indol-4-amine (97 mg, 0.16 mmol) in THF (2 mL) and add 6M aqueous HCl (2 mL). Stir at ambient temperature for 1 hr. Dilute with water, quench with saturated aqueous sodium bicarbonate, extract with EtOAc, wash with saturated aqueous NaCl, dry over anhydrous sodium sulfate, filter, and concentrate under vacuum. Purify by prep-HPLC to give 3-[[6,7-dichloro-3-(1H-pyrazol-4-yl)-1H-indol-4-yl]amino]-2,2-difluoro-propan-1-ol (33.53 mg, 58%) as a white solid. ES/MS (m/z): (³⁵Cl/³⁷Cl) 361.2/363.2 (M+H).

### Intermediate 445

### N-[3-[tert-Butyl(diphenyl)silyl]oxy-2-fluoro-propyl]-6, 7-dichloro-3-(1H-pyrazol-4-yl)-1H-indol-4-amine

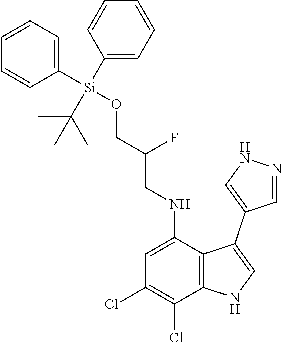 [SMILES: CC(C)(C)[Si](OCC(F)CNc1cc(Cl)c(Cl)c2[nH]cc(-c3cn[nH]c3)c12)(c1ccccc1)c1ccccc1]

Dissolve 6,7-dichloro-3-(1H-pyrazol-4-yl)-1H-indol-4-amine (140 mg, 0.438 mmol, HCl salt) in MeOH (3 mL) and add DIPEA (0.23 mL, 1.3 mmol) to adjust to pH=9, then stir at ambient temperature for 15 min. Add acetic acid (55 mg, 0.91 mmol) to adjust pH=5 and stir at ambient temperature for 30 min. Add 3-[tert-butyl(diphenyl)silyl]oxy-2-fluoro-propanal (CAS: 151021-56-6, 240 mg, 0.654 mmol) and stir at ambient temperature for 30 min. Add NaBH₃CN (88 mg, 1.3 mmol) and stir at ambient temperature for 16-18 hrs. Concentrate under vacuum and dilute with water. Adjust to pH=7 with saturated aqueous sodium bicarbonate, extract with DCM, wash with saturated aqueous NaCl, dry over anhydrous sodium sulfate, filter, and concentrate under vacuum. Purify by flash column chromatography eluting with EtOAc in petroleum ether to give N-[3-[tert-butyl(diphenyl)silyl]oxy-2-fluoro-propyl]-6,7-dichloro-3-(1H-pyrazol-4-yl)-1H-indol-4-amine (70 mg, 27%) as a light-yellow oil. ES/MS (m/z): (³⁵Cl/³⁷Cl) 581.1/583.1 (M+H).

### EXAMPLE 187

### 3-[[6,7-Dichloro-3-(1H-pyrazol-4-yl)-1H-indol-4-yl]amino]-2-fluoro-propan-1-ol

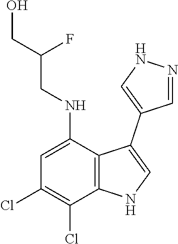 [SMILES: OCC(F)CNc1cc(Cl)c(Cl)c2[nH]cc(-c3cn[nH]c3)c12]

Dissolve N-[3-[tert-butyl(diphenyl)silyl]oxy-2-fluoro-propyl]-6,7-dichloro-3-(1H-pyrazol-4-yl)-1H-indol-4-amine (70 mg, 0.12 mmol) in THF (2 mL) and add TBAF (0.16 mL, 0.16 mmol, 1M in THF) at 0° C., then stir at ambient temperature for 1 hr. Dilute with EtOAc, wash sequentially with saturated aqueous NH₄Cl and saturated aqueous NaCl, dry over anhydrous sodium sulfate, filter, and concentrate under vacuum. Purify by reverse phase prep-HPLC to give 3-[[6,7-dichloro-3-(1H-pyrazol-4-yl)-1H-indol-4-yl]amino]-2-fluoro-propan-1-ol (12.92 mg, 31%) as a white solid. ES/MS (m/z): (³⁵Cl/³⁷Cl) 343.3/345.2 (M+H).

### Intermediate 446

### 6,7-Dichloro-N-(3,3-difluoropropyl)-3-(1-tetrahydropyran-2-ylpyrazol-4-yl)-1H-indol-4-amine

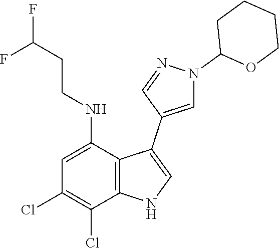 [SMILES: FC(F)CCNc1cc(Cl)c(Cl)c2[nH]cc(-c3cnn(C4CCCCO4)c3)c12]

Suspend 4-bromo-6,7-dichloro-1-(p-tolylsulfonyl)-3-(1-tetrahydropyran-2-ylpyrazol-4-yl)indole (300 mg, 0.501 mmol), 3,3-difluoropropan-1-amine (105 mg, 0.758 mmol, HCl salt), t-BuONa (150 mg, 1.51 mmol), (t-Bu₃P)₂Pd (40 mg, 0.077 mmol) in 1,4-dioxane (6 mL). Purge with N₂ and heat to 100° C. for 16-18 hrs. Cool to ambient temperature and concentrate under vacuum. Purify by flash column chromatography eluting with EtOAc in petroleum ether to give 6,7-dichloro-N-(3,3-difluoropropyl)-3-(1-tetrahydropyran-2-ylpyrazol-4-yl)-1H-indol-4-amine (150 mg, 52%) as a yellow gum. ES/MS (m/z): (³⁵Cl/³⁷Cl) 429.1/431.1 (M+H).

### EXAMPLE 188

### 6,7-Dichloro-N-(3,3-difluoropropyl)-3-(1H-pyrazol-4-yl)-1H-indol-4-amine

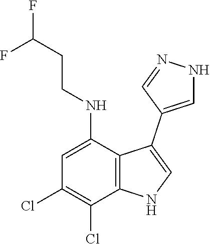 [SMILES: FC(F)CCNc1cc(Cl)c(Cl)c2[nH]cc(-c3cn[nH]c3)c12]

Dissolve 6,7-dichloro-N-(3,3-difluoropropyl)-3-(1-tetrahydropyran-2-ylpyrazol-4-yl)-1H-indol-4-amine (150 mg, 0.262 mmol) in DCM (2 mL) and add TFA (2 mL). Stir at ambient temperature for 2 hrs. Concentrate under vacuum and purify by prep-HPLC to give 6,7-dichloro-N-(3,3-difluoropropyl)-3-(1H-pyrazol-4-yl)-1H-indol-4-amine (71.26 mg, 78%) as a gray solid. ES/MS (m/z): (³⁵Cl/³⁷Cl) 345.2/347.2 (M+H).

### Intermediate 447

### 3,4-Dichloro-10-(1H-pyrazol-4-yl)-6,7,8,9-tetrahydropyrido[1,2-a]indol-7-amine

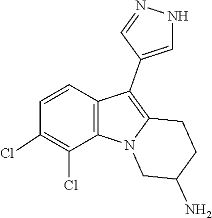 [SMILES: NC1CCc2c(-c3cn[nH]c3)c3ccc(Cl)c(Cl)c3n2C1]

Dissolve 3,4-dichloro-10-(1-tetrahydropyran-2-ylpyrazol-4-yl)-6,7,8,9-tetrahydro pyrido[1,2-a]indol-7-amine (81 mg, 0.19 mmol) in 4M HCl in dioxane (3 mL) and stir at ambient temperature for 1 hr. Concentrate under vacuum to give 3,4-dichloro-10-(1H-pyrazol-4-yl)-6,7,8,9-tetrahydropyrido[1,2-a]indol-7-amine (148 mg, 99+%, HCl salt) as a yellow solid. ES/MS (m/z): (³⁵Cl/³⁷Cl) 321.1/323.1 (M+H).

### EXAMPLE 189

### N-[3,4-Dichloro-10-(1H-pyrazol-4-yl)-6,7,8,9-tetrahydropyrido[1,2-a]indol-7-yl]-2-hydroxy-2-methyl-propanamide

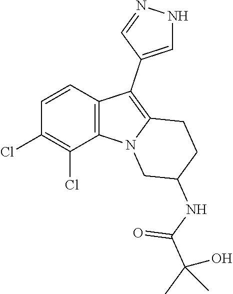 [SMILES: CC(C)(O)C(=O)NC1CCc2c(-c3cn[nH]c3)c3ccc(Cl)c(Cl)c3n2C1]

Dissolve 3,4-dichloro-10-(1H-pyrazol-4-yl)-6,7,8,9-tetrahydropyrido[1,2-a]indol-7-amine (148 mg, 0.346 mmol, HCl salt), 2-hydroxyisobutyric acid (75 mg, 0.70 mmol), DIPEA (0.301 mL, 1.73 mmol) and HATU (270 mg, 0.696 mmol) in DMF (2 mL) and stir at ambient temperature for 16-18 hrs. Concentrate under vacuum and purify by prep-HPLC to give N-[3,4-dichloro-10-(1H-pyrazol-4-yl)-6,7,8,9-tetrahydropyrido[1,2-a]indol-7-yl]-2-hydroxy-2-methyl-propanamide (20.48 mg, 16%) as a white solid. ES/MS (m/z): (³⁵Cl/³⁷Cl) 407.3/409.3 (M+H).

### EXAMPLE 190

### N-[3,4-Dichloro-10-(1H-pyrazol-4-yl)-6,7,8,9-tetrahydropyrido[1,2-a]indol-7-yl]-4-hydroxy-tetrahydropyran-4-carboxamide

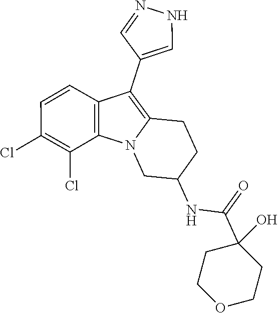 [SMILES: O=C(NC1CCc2c(-c3cn[nH]c3)c3ccc(Cl)c(Cl)c3n2C1)C1(O)CCOCC1]

Dissolve 3,4-dichloro-10-(1H-pyrazol-4-yl)-6,7,8,9-tetrahydropyrido[1,2-a]indol-7-amine (81 mg, 0.22 mmol, HCl salt), 4-hydroxyoxane-4-carboxylic acid (70 mg, 0.46 mmol), DIPEA (0.20 mL, 1.15 mmol) and HATU (175 mg, 0.450 mmol) in DMF (2 mL) and stir at ambient temperature for 16-18 hrs. Concentrate under vacuum and purify by prep-HPLC to give N-[3,4-dichloro-10-(1H-pyrazol-4-yl)-6,7,8,9-tetrahydropyrido[1,2-a]indol-7-yl]-4-hydroxy-tetrahydropyran-4-carboxamide (41.66 mg, 42%) as a white solid. ES/MS (m/z): (³⁵Cl/³⁷Cl) 449.3/451.3 (M+H).

### Intermediate 448

### 3,4-Dichloro-N-pyrimidin-2-yl-10-(1-tetrahydropyran-2-ylpyrazol-4-yl)-6, 7, 8,9-tetrahydropyrido[1,2-a]indol-7-amine

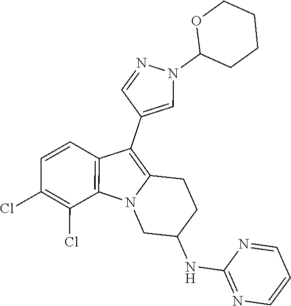 [SMILES: Clc1ccc2c(-c3cnn(C4CCCCO4)c3)c3n(c2c1Cl)CC(Nc1ncccn1)CC3]

Dissolve 3,4-dichloro-10-(1-tetrahydropyran-2-ylpyrazol-4-yl)-6,7,8,9-tetrahydropyrido[1,2-a]indol-7-amine (100 mg, 0.234 mmol), 2-chloropyrimidine (85 mg, 0.72 mmol) and DIPEA (0.085 mL, 0.49 mmol) in IPA (2 mL) and stir at 120° C. for 4 hrs in a sealed tube under microwave irradiation. Concentrate under vacuum, dilute with EtOAc, and wash with water. Extract the aqueous layer with EtOAc (3×). Wash the combined organic layers with saturated aqueous NaCl, dry over sodium sulfate, filter, and concentrate under vacuum. Purify by flash column chromatography eluting with EtOAc in petroleum ether to give 3,4-dichloro-N-pyrimidin-2-yl-10-(1-tetrahydropyran-2-ylpyrazol-4-yl)-6,7,8,9-tetrahydropyrido[1,2-a]indol-7-amine (50 mg, 42%) as a yellow solid. ES/MS (m/z): (³⁵Cl/³⁷Cl) 483.1/485.1 (M+H).

### EXAMPLE 191

### 3,4-Dichloro-10-(1H-pyrazol-4-yl)-N-pyrimidin-2-yl-6,7,8,9-tetrahydropyrido[1,2-a]indol-7-amine

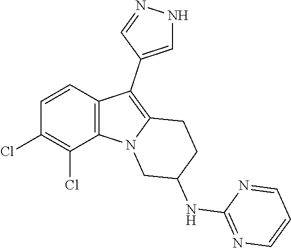 [SMILES: Clc1ccc2c(-c3cn[nH]c3)c3n(c2c1Cl)CC(Nc1ncccn1)CC3]

Dissolve 3,4-dichloro-N-pyrimidin-2-yl-10-(1-tetrahydropyran-2-ylpyrazol-4-yl)-6,7,8,9-tetrahydropyrido[1,2-a]indol-7-amine (59 mg, 0.11 mmol) in DCM (2 mL) and add TFA (2 mL), then stir at ambient temperature for 2 hrs. Concentrate under vacuum and purify by prep-HPLC to give 3,4-dichloro-10-(1H-pyrazol-4-yl)-N-pyrimidin-2-yl-6,7,8,9-tetrahydropyrido[1,2-a]indol-7-amine (14.04 mg, 32%) as a pale-yellow solid. ES/MS (m/z): (³⁵Cl/³⁷Cl) 399.3/401.2 (M+H).

### Intermediate 449

### 6,7-Dichloro-N-[(2,2-dimethyl-1,3-dioxan-5-yl)methyl]-3-(1H-pyrazol-4-yl)-1H-indol-4-amine

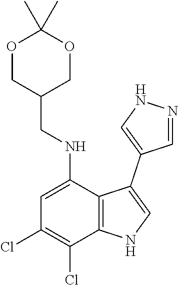 [SMILES: CC1(C)OCC(CNc2cc(Cl)c(Cl)c3[nH]cc(-c4cn[nH]c4)c23)CO1]

Dissolve 6,7-dichloro-3-(1H-pyrazol-4-yl)-1H-indol-4-amine (50 mg, 0.18 mmol) in MeOH (4 mL) and add acetic acid (11 mg, 0.18 mmol) to adjust to pH=5. Stir at ambient temperature for 0.5 hr. Add 2,2-dimethyl-1,3-dioxane-5-carbaldehyde (81 mg, 0.53 mmol) and stir at ambient temperature for 0.5 hr, follow by adding sodium cyanoborohydride (35 mg, 0.53 mmol) and stir at ambient temperature for 16 hrs. Dilute with water, extract with EtOAc, wash with saturated aqueous NaCl, dry over anhydrous sodium sulfate, filter, and concentrate under vacuum to give 6,7-dichloro-N-[(2,2-dimethyl-1,3-dioxan-5-yl)methyl]-3-(1H-pyrazol-4-yl)-1H-indol-4-amine (100 mg, 86%) as a brown oil. ES/MS (m/z): (³⁵Cl/³⁷Cl) 394.9/397.0 (M+H).

### EXAMPLE 192

### 2-[[[6,7-Dichloro-3-(1H-pyrazol-4-yl)-1H-indol-4-yl]amino]methyl]propane-1,3-diol

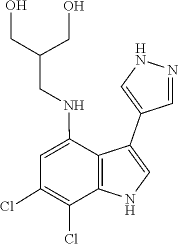 [SMILES: OCC(CO)CNc1cc(Cl)c(Cl)c2[nH]cc(-c3cn[nH]c3)c12]

Suspend 6,7-dichloro-N-[(2,2-dimethyl-1,3-dioxan-5-yl)methyl]-3-(1H-pyrazol-4-yl)-1H-indol-4-amine (70 mg, 0.17 mmol) in THF (2 mL) and add 6M aqueous HCl (2 mL). Stir for 1 hr at ambient temperature and concentrate under vacuum. Purify by prep-HPLC to give 2-[[[6,7-dichloro-3-(1H-pyrazol-4-yl)-1H-indol-4-yl]amino]methyl]propane-1,3-diol (10.1 mg, 16%) as a white solid. ES/MS (m/z): (³⁵Cl/³⁷Cl) 355.2/357.2 (M+H).

### Intermediate 450

### tert-Butyl-[3-[6,7-dichloro-1-(p-tolylsulfonyl)indol-4-yl]oxypropoxy]-dimethyl-silane

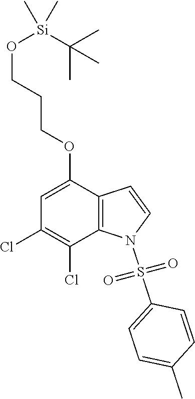 [SMILES: Cc1ccc(S(=O)(=O)n2ccc3c(OCCCO[Si](C)(C)C(C)(C)C)cc(Cl)c(Cl)c32)cc1]

Dissolve 6,7-dichloro-1-(p-tolylsulfonyl)indol-4-ol (500 mg, 1.26 mmol) in DMF (5 mL). Cool to 0° C. Add (3-bromopropoxy)-tert-butyldimethylsilane (675 mg, 2.53 mmol) and potassium carbonate (350 mg, 2.53 mmol) and stir at ambient temperature for 16-18 hrs. Dilute with EtOAc, and wash with water. Extract the aqueous layer with EtOAc (3×). Wash the combined organic layers with saturated aqueous NaCl, dry over sodium sulfate, filter, and concentrate under vacuum. Purify by flash column chromatography eluting with EtOAc in petroleum ether to give tert-butyl-[3-[6,7-dichloro-1-(p-tolylsulfonyl)indol-4-yl]oxypropoxy]-dimethyl-silane (708 mg, 90%) as a colorless oil.

### Intermediate 451

### 3-[(6,7-Dichloro-1H-indol-4-yl)oxy]propan-1-ol

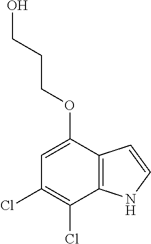 [SMILES: OCCCOc1cc(Cl)c(Cl)c2[nH]ccc12]

Dissolve tert-butyl-[3-[6,7-dichloro-1-(p-tolylsulfonyl)indol-4-yl]oxypropoxy]-dimethyl-silane (708 mg, 1.14 mmol) in THF (3 mL) and add TBAF (5 mL, 1.0M in THF). Stir at ambient temperature for 2 hrs. Dilute with EtOAc, wash with saturated aqueous NH₄Cl, dry over anhydrous sodium sulfate, filter, and concentrate under vacuum. Purify by flash column chromatography eluting with EtOAc in petroleum ether to give 3-[(6,7-dichloro-1H-indol-4-yl)oxy]propan-1-ol (237 mg, 77%) as a white solid. ES/MS (m/z): (³⁵Cl/³⁷Cl) 260.1/262.1 (M+H).

### Intermediate 452

### 3-[(6,7-Dichloro-3-iodo-1H-indol-4-yl)oxy]propan-1-ol

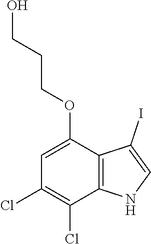 [SMILES: OCCCOc1cc(Cl)c(Cl)c2[nH]cc(I)c12]

Dissolve 3-[(6,7-dichloro-1H-indol-4-yl)oxy]propan-1-ol (237 mg, 0.875 mmol) in DMF (3 mL). Add NIS (241 mg, 1.05 mmol) and stir at ambient temperature for 1.5 hr. Dilute with EtOAc and wash with water. Extract the aqueous layer with EtOAc (3×). Wash the combined organic layers with saturated aqueous NaCl, dry over sodium sulfate, filter, and concentrate under vacuum. Purify by flash column chromatography eluting with EtOAc in petroleum ether to give 3-[(6,7-dichloro-3-iodo-1H-indol-4-yl)oxy]propan-1-ol (94 mg, 25%) as a purple solid.

### Intermediate 453

### 3-[[6,7-Dichloro-3-(1-tetrahydropyran-2-ylpyrazol-4-yl)-1H-indol-4-yl]oxy]propan-1-ol

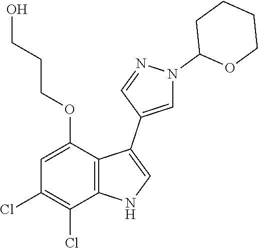 [SMILES: OCCCOc1cc(Cl)c(Cl)c2[nH]cc(-c3cnn(C4CCCCO4)c3)c12]

Suspend 3-[(6,7-dichloro-3-iodo-1H-indol-4-yl)oxy]propan-1-ol (94 mg, 0.22 mmol), 1-(tetrahydro-2H-pyran-2-yl)-4-(4,4,5,5-tetramethyl-1,3,2-dioxaborolan-2-yl)-1H-pyrazole (97 mg, 0.33 mmol), Pd(dtbpf)Cl₂ (15 mg, 0.023 mmol) and sodium carbonate (70 mg, 0.66 mmol) in 1,4-dioxane (2 mL) and water (0.5 mL). Purge with N₂ and heat at 90° C. for 1 hr. Cool to ambient temperature, dilute with water and extract with EtOAc, wash with saturated aqueous NaCl, dry over sodium sulfate, filter, and concentrate under vacuum. Purify by flash column chromatography eluting with EtOAc in petroleum ether to give 3-[[6,7-dichloro-3-(1-tetrahydropyran-2-ylpyrazol-4-yl)-1H-indol-4-yl]oxy]propan-1-ol (96 mg, 85%) as a yellow oil. ES/MS (m/z): (³⁵Cl/³⁷Cl) 410.0/411.9 (M+H).

### EXAMPLE 193

### 3-[[6,7-Dichloro-3-(1H-pyrazol-4-yl)-1H-indol-4-yl]oxy]propan-1-ol

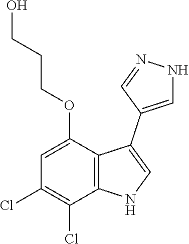 [SMILES: OCCCOc1cc(Cl)c(Cl)c2[nH]cc(-c3cn[nH]c3)c12]

Dissolve 3-[[6,7-dichloro-3-(1-tetrahydropyran-2-ylpyrazol-4-yl)-1H-indol-4-yl]oxy]propan-1-ol (96 mg, 0.19 mmol) in THF (2 mL) and add 6M aqueous HCl (2 mL), then stir at ambient temperature for 2 hrs. Concentrate under vacuum and purify by prep-HPLC to give 3-[[6,7-dichloro-3-(1H-pyrazol-4-yl)-1H-indol-4-yl]oxy]propan-1-ol (21 mg, 34%) as a white solid. ES/MS (m/z): (³⁵Cl/³⁷Cl) 326.3/328.2 (M+H).

### Intermediate 454

### 6,7-Dichloro-4-[(2,2-dimethyl-1,3-dioxan-5-yl)methoxy]-1-(p-tolylsulfonyl)indole

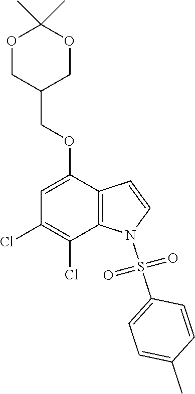 [SMILES: Cc1ccc(S(=O)(=O)n2ccc3c(OCC4COC(C)(C)OC4)cc(Cl)c(Cl)c32)cc1]

Dissolve 6,7-dichloro-1-(p-tolylsulfonyl)indol-4-ol (500 mg, 1.33 mmol), (2,2-dimethyl-1,3-dioxan-5-yl)methanol (411 mg, 2.67 mmol), Ph₃P (893 mg, 3.34 mmol) in toluene (5 mL). Purge with N₂ and add DIAD (0.42 mL, 2.0 mmol). Stir at 110° C. for 16-18 hrs. Quench with water, extract with EtOAc, dry over anhydrous sodium sulfate, filter, and concentrate under vacuum. Purify by flash column chromatography eluting with EtOAc in petroleum ether to give 6,7-dichloro-4-[(2,2-dimethyl-1,3-dioxan-5-yl)methoxy]-1-(p-tolylsulfonyl)indole (1.24 g, 100%) as a colorless oil.

### Intermediate 455

### 6,7-Dichloro-4-[(2,2-dimethyl-1,3-dioxan-5-yl)methoxy]-1H-indole

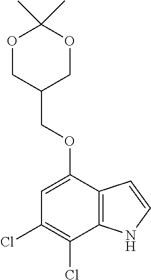 [SMILES: CC1(C)OCC(COc2cc(Cl)c(Cl)c3[nH]ccc23)CO1]

Dissolve 6,7-dichloro-4-[(2,2-dimethyl-1,3-dioxan-5-yl)methoxy]-1-(p-tolylsulfonyl)indole (1.24 g, 2.30 mmol) in THF (5 mL). Add TBAF (7 mL, 1.0M in THF) and stir at ambient temperature for 2 hrs. Dilute with EtOAc, wash with saturated aqueous NH₄Cl, dry over anhydrous sodium sulfate, filter, and concentrate under vacuum. Purify by flash column chromatography eluting with EtOAc in petroleum ether to give 6,7-dichloro-4-[(2,2-dimethyl-1,3-dioxan-5-yl)methoxy]-1H-indole (444 mg, 46%) as a colorless oil. ES/MS (m/z): (³⁵Cl/³⁷Cl) 330.0/332.0 (M+H).

### Intermediate 456

### 6,7-Dichloro-4-[(2,2-dimethyl-1,3-dioxan-5-yl)methoxy]-3-iodo-1H-indole

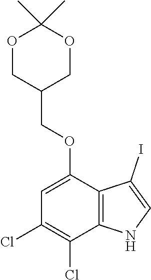 [SMILES: CC1(C)OCC(COc2cc(Cl)c(Cl)c3[nH]cc(I)c23)CO1]

Dissolve 6,7-dichloro-4-[(2,2-dimethyl-1,3-dioxan-5-yl)methoxy]-1H-indole (444 mg, 1.05 mmol) in DMF (3 mL). Add NIS (289 mg, 1.26 mmol) and stir at ambient temperature for 1.5 hr. Dilute with EtOAc, and wash with water. Extract the aqueous layer with EtOAc (3×). Wash the combined organic layers with saturated aqueous NaCl, dry over sodium sulfate, filter, and concentrate under vacuum. Purify by flash column chromatography eluting with EtOAc in petroleum ether to give 6,7-dichloro-4-[(2,2-dimethyl-1,3-dioxan-5-yl)methoxy]-3-iodo-1H-indole (344 mg, 68%) as a white solid.

### Intermediate 457

### 6,7-Dichloro-4-[(2,2-dimethyl-1,3-dioxan-5-yl)methoxy]-3-(1-tetrahydropyran-2-ylpyrazol-4-yl)-1H-indole

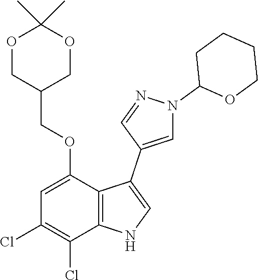 [SMILES: CC1(C)OCC(COc2cc(Cl)c(Cl)c3[nH]cc(-c4cnn(C5CCCCO5)c4)c23)CO1]

Suspend 6,7-dichloro-4-[(2,2-dimethyl-1,3-dioxan-5-yl)methoxy]-3-iodo-1H-indole (344 mg, 0.716 mmol), 1-(tetrahydro-2H-pyran-2-yl)-4-(4,4,5,5-tetramethyl-1,3,2-dioxaborolan-2-yl)-1H-pyrazole (315 mg, 1.08 mmol), Pd(dtbpf)Cl₂ (50 mg, 0.076 mmol) and sodium carbonate (230 mg, 2.17 mmol) in 1,4-dioxane (4 mL) and water (1 mL). Purge with N₂ and heat at 90° C. for 1 hr. Cool to ambient temperature, dilute with water and extract with EtOAc, wash with saturated aqueous NaCl, dry over sodium sulfate, filter, and concentrate under vacuum. Purify by flash column chromatography eluting with EtOAc in petroleum ether to give 6,7-dichloro-4-[(2,2-dimethyl-1,3-dioxan-5-yl)methoxy]-3-(1-tetrahydropyran-2-ylpyrazol-4-yl)-1H-indole (257 mg, 59%) as a yellow oil. ES/MS (m/z): (³⁵Cl/³⁷Cl) 480.2/482.2 (M+H).

### EXAMPLE 194

### 2-[[6,7-Dichloro-3-(1H-pyrazol-4-yl)-1H-indol-4-yl]oxymethyl]propane-1,3-diol

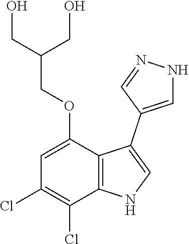 [SMILES: OCC(CO)COc1cc(Cl)c(Cl)c2[nH]cc(-c3cn[nH]c3)c12]

Dissolve 6,7-dichloro-4-[(2,2-dimethyl-1,3-dioxan-5-yl)methoxy]-3-(1-tetrahydropyran-2-ylpyrazol-4-yl)-1H-indole (257 mg, 0.423 mmol) in THF (2 mL) and add 6M aqueous HCl (4 mL), then stir at ambient temperature for 2 hrs. Concentrate under vacuum and purify by prep-HPLC to give 2-[[6,7-dichloro-3-(1H-pyrazol-4-yl)-1H-indol-4-yl]oxymethyl]propane-1,3-diol (44.65 mg, 29%) as an off-white solid. ES/MS (m/z): (³⁵Cl/³⁷Cl) 356.2/358.2 (M+H).

### Intermediate 458

### tert-Butyl (6, 7-dichloro-3-(1-(tetrahydro-2H-pyran-2-yl)-1H-pyrazol-4-yl)-1H-indol-4-yl)carbamate

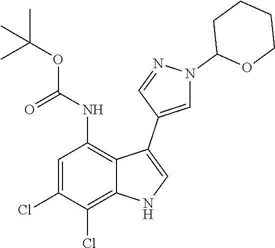 [SMILES: CC(C)(C)OC(=O)Nc1cc(Cl)c(Cl)c2[nH]cc(-c3cnn(C4CCCCO4)c3)c12]

Combine tert-butyl (6,7-dichloro-3-iodo-1H-indol-4-yl)carbamate (7 g, 13 mmol), 1-tetrahydropyran-2-yl-4-(4,4,5,5-tetramethyl-1,3,2-dioxaborolan-2-yl)pyrazole (7.5 g, 26 mmol), sodium carbonate (4.2 g, 39 mmol), and Pd(dtbpf)Cl₂ (1.8 g, 2.7 mmol) in 1,4-dioxane (100 mL) and water (25 mL). Purge with N₂ and stir at 90° C. for 2 h. Filter and concentrate under vacuum. Purify by flash column chromatography eluting with EtOAc in petroleum ether to give tert-butyl (6,7-dichloro-3-(1-(tetrahydro-2H-pyran-2-yl)-1H-pyrazol-4-yl)-1H-indol-4-yl)carbamate (6 g, 73%) as a brown solid. ES/MS (m/z): (³⁵Cl/³⁷Cl) 451.2/453.1 (M+H).

### Intermediate 458A

### tert-Butyl N-[2-[4-(tert-butoxycarbonylamino)-6,7-dichloro-3-(1-tetrahydropyran-2-ylpyrazol -4-yl)indol -1-yl]ethyl]carbamate

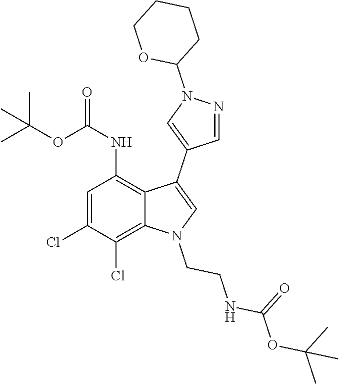 [SMILES: CC(C)(C)OC(=O)NCCn1cc(-c2cnn(C3CCCCO3)c2)c2c(NC(=O)OC(C)(C)C)cc(Cl)c(Cl)c21]

Suspend tert-butyl N-[6,7-dichloro-3-(1-tetrahydropyran-2-ylpyrazol-4-yl)-1H-indol-4-yl]carbamate (1.00 g, 1.57 mmol) and Cs₂CO₃ (1.38 g, 4.24 mmol) in DMF (10 mL), then stir at ambient temperature for 1 hr. Add tert-butyl-1,2,3-oxathiazolidine-3-carboxylate 2,2-dioxide (745 mg, 3.17 mmol) and stir at ambient temperature for 16-18 hrs. Quench with 1M aqueous HCl at 0° C., extract with EtOAc, wash with saturated aqueous NaCl, dry over anhydrous sodium sulfate, filter, and concentrate under vacuum. Purify by flash column chromatography eluting with EtOAc in petroleum ether to give tert-butyl N-[2-[4-(tert-butoxycarbonylamino)-6,7-dichloro-3-(1-tetrahydropyran-2-ylpyrazol-4-yl)indol-1-yl]ethyl]carbamate (690 mg, 66%) as a light-yellow solid.

### Intermediate 459

### 1-(2-Aminoethyl)-6,7-dichloro-3-(1H-pyrazol-4-yl)indol-4-amine hydrochloride

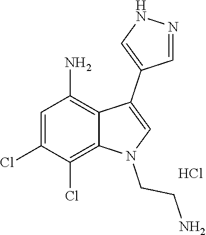 [SMILES: NCCn1cc(-c2cn[nH]c2)c2c(N)cc(Cl)c(Cl)c21 ; Cl]

Dissolve tert-butyl N-[2-[4-(tert-butoxycarbonylamino)-6,7-dichloro-3-(1-tetrahydropyran-2-ylpyrazol-4-yl)indol-1-yl]ethyl]carbamate (690 mg, 1.05 mmol) in 4M HCl in EtOAc (6 mL) and stir at ambient temperature for 1 hr. Concentrate and triturate with MeOH to give 1-(2-aminoethyl)-6,7-dichloro-3-(1H-pyrazol-4-yl)indol-4-amine hydrochloride (360 mg, 89%) as a light-yellow solid. ES/MS (m/z): (³⁵Cl/³⁷Cl) 309.9/311.7 (M+H).

### Intermediate 460

### N-[2-[4-Amino-6,7-dichloro-3-(1H-pyrazol-4-yl)indol-1-yl]ethyl]acetamide

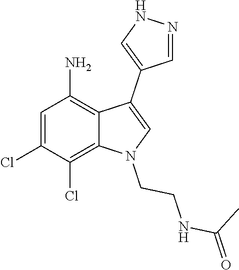 [SMILES: CC(=O)NCCn1cc(-c2cn[nH]c2)c2c(N)cc(Cl)c(Cl)c21]

Dissolve 1-(2-aminoethyl)-6,7-dichloro-3-(1H-pyrazol-4-yl)indol-4-amine hydrochloride (360 mg, 0.935 mmol) and TEA (0.95 mL, 6.8 mmol) in DCM (6 mL). Add Ac₂O (0.135 mL, 1.39 mmol) at 0° C. and stir at 0° C. for 2 hrs. Quench with saturated aqueous sodium carbonate, extract with EtOAc, wash with saturated aqueous NaCl, dry over anhydrous sodium sulfate, filter, and concentrate under vacuum to give N-[2-[4-amino-6,7-dichloro-3-(1H-pyrazol-4-yl)indol-1-yl]ethyl]acetamide (350 mg, 99%) as an off-white solid. ES/MS (m/z): (³⁵Cl/³⁷Cl) 351.9/353.9 (M+H).

### Intermediate 461

### N-[2-[4-[3-[tert-Butyl(dimethyl)silyl]oxypropyl amino]-6, 7-dichloro-3-(1H-pyrazol-4-yl)indol-1-yl]ethyl]acetamide

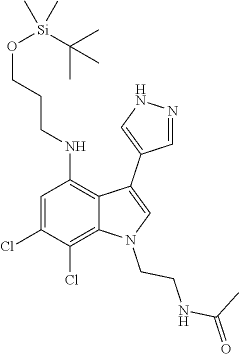 [SMILES: CC(=O)NCCn1cc(-c2cn[nH]c2)c2c(NCCCO[Si](C)(C)C(C)(C)C)cc(Cl)c(Cl)c21]

Dissolve N-[2-[4-amino-6,7-dichloro-3-(1H-pyrazol-4-yl)indol-1-yl]ethyl]acetamide (150 mg, 0.426 mmol) in MeOH (3 mL) and add acetic acid (50 mg, 0.83 mmol) to adjust to pH=5, then stir at ambient temperature for 30 min. Add 3-[tert-butyl(dimethyl)silyl]oxypropanal (140 mg, 0.741 mmol) and stir at ambient temperature for another 30 min. Add NaBH₃CN (75 mg, 1.13 mmol) and stir at ambient temperature for 16-18 hrs. Combine with another batch run at 0.19×scale for work up and purification. Concentrate under vacuum and dilute with DCM. Adjust to pH=7 with saturated aqueous sodium bicarbonate, extract with DCM, wash with saturated aqueous NaCl, dry over anhydrous sodium sulfate, filter, and concentrate under vacuum. Purify by flash column chromatography eluting with MeOH in DCM to give N-[2-[4-[3-[tert-butyl(dimethyl)siyl]oxypropylamino]-6,7-dichloro-3-(1H-pyrazol-4-yl)indol-1-yl]ethyl]acetamide (130 mg, 39%) as a light-yellow oil. ES/MS (m/z): (³⁵Cl/³⁷Cl) 523.9/526.1 (M+H).

### EXAMPLE 195

### N-[2-[6,7-Dichloro-4-(3-hydroxypropylamino)-3-(1H-pyrazol-4-yl)indol-1-yl]ethyl]acetamide

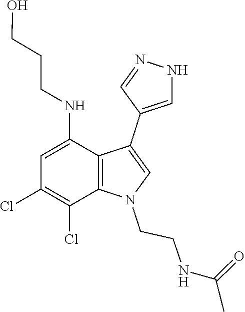 [SMILES: CC(=O)NCCn1cc(-c2cn[nH]c2)c2c(NCCCO)cc(Cl)c(Cl)c21]

Dissolve N-[2-[4-[3-[tert-butyl(dimethyl)silyl]oxypropylamino]-6,7-dichloro-3-(1H-pyrazol-4-yl)indol-1-yl]ethyl]acetamide (130 mg, 0.198 mmol) in 4M HCl in MeOH (3 mL) and stir at ambient temperature for 1 hr. Quench with saturated aqueous sodium bicarbonate, extract with EtOAc, wash with saturated aqueous NaCl, dry over anhydrous sodium sulfate, filter, and concentrate under vacuum. Purify by prep-HPLC to give N-[2-[6,7-dichloro-4-(3-hydroxypropylamino)-3-(1H-pyrazol-4-yl)indol-1-yl]ethyl]acetamide (57.20 mg, 68%) as a red solid. ES/MS (m/z): (³⁵Cl/³⁷Cl) 410.3/412.3 (M+H).

### Intermediate 462

### tert-Butyl N-[2-[[3,4-dichloro-10-(1-tetrahydropyran-2-ylpyrazol-4-yl)-6,7,8,9-tetrahydropyrido[1,2-a]indol-7-yl]amino]-2-oxo-ethyl]carbamate

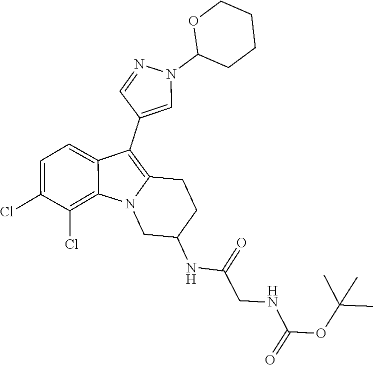 [SMILES: CC(C)(C)OC(=O)NCC(=O)NC1CCc2c(-c3cnn(C4CCCCO4)c3)c3ccc(Cl)c(Cl)c3n2C1]

Dissolve 3,4-dichloro-10-(1-tetrahydropyran-2-ylpyrazol-4-yl)-6,7,8,9-tetrahydropyrido[1,2-a]indol-7-amine (150 mg, 0.352 mmol), Boc-Gly-OH (125 mg, 0.699 mmol) and DIPEA (0.3 mL, 2 mmol) in DMF (3 mL). Add HATU (273 mg, 0.704 mmol). Stir at ambient temperature for 2 hrs. Dilute with water, extract with EtOAc, dry over anhydrous sodium sulfate, filter, and concentrate under vacuum. Purify by flash column chromatography eluting with MeOH in DCM to give tert-butyl N-[2-[[3,4-dichloro-10-(1-tetrahydropyran-2-ylpyrazol-4-yl)-6,7,8,9-tetrahydropyrido[1,2-a]indol-7-yl]amino]-2-oxo-ethyl]carbamate (200 mg, 74%) as a yellow gum. ES/MS (m/z): (³⁵Cl/³⁷Cl) 562.2/564.1 (M+H).

### Intermediate 463

### 2-Amino-N-[3,4-dichloro-10-(1H-pyrazol-4-yl)-6,7,8,9-tetrahydropyrido[1,2-a]indol-7-yl]acetamide

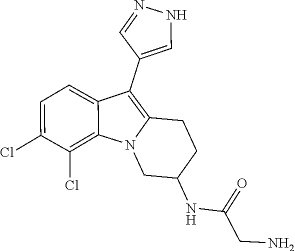 [SMILES: NCC(=O)NC1CCc2c(-c3cn[nH]c3)c3ccc(Cl)c(Cl)c3n2C1]

Dissolve tert-butyl N-[2-[[3,4-dichloro-10-(1-tetrahydropyran-2-ylpyrazol-4-yl)-6,7,8,9-tetrahydropyrido[1,2-a]indol-7-yl]amino]-2-oxo-ethyl]carbamate (200 mg, 0.260 mmol) in DCM (3 mL) and add TFA (3 mL). Stir at ambient temperature for 2 hrs. Concentrate under vacuum, dilute with water, adjust to pH=12 with 2N aqueous NaOH, extract with EtOAc, dry over anhydrous sodium sulfate, filter, and concentrate under vacuum to give 2-amino-N-[3,4-dichloro-10-(1H-pyrazol-4-yl)-6,7,8,9-tetrahydropyrido[1,2-a]indol-7-yl]acetamide (100 mg, 87%) as a yellow solid.

### Intermediate 464

### tert-Butyl N-[6-[4-[7-[[2-[6-(tert-butoxycarbonylamino)hexanoylamino]acetyl]amino]-3,4-dichloro-6,7,8,9-tetrahydropyrido[1,2-a]indol-10-yl]pyrazol-1-yl]-6-oxo-hexyl]carbamate

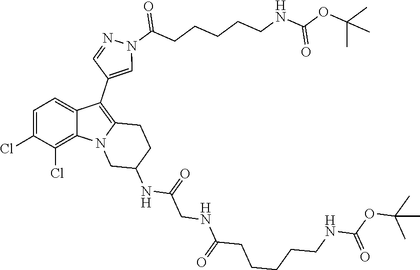 [SMILES: CC(C)(C)OC(=O)NCCCCCC(=O)NCC(=O)NC1CCc2c(-c3cnn(C(=O)CCCCCNC(=O)OC(C)(C)C)c3)c3ccc(Cl)c(Cl)c3n2C1]

Dissolve 2-amino-N-[3,4-dichloro-10-(1H-pyrazol-4-yl)-6,7,8,9-tetrahydropyrido[1,2-a]indol-7-yl]acetamide (80 mg, 0.18 mmol), Boc-6-aminohexanoic acid (85 mg, 0.36 mmol) and DIPEA (0.16 mL, 0.92 mmol) in DMF (2 mL) and add HATU (140 mg, 0.361 mmol). Stir at ambient temperature for 2 hrs. Dilute with water, extract with EtOAc, dry over anhydrous sodium sulfate, filter, and concentrate under vacuum. Purify by flash column chromatography eluting with EtOAc in petroleum ether to give tert-butyl N-[6-[4-[7-[[2-[6-(tert-butoxycarbonylamino)hexanoylamino]acetyl]amino]-3,4-dichloro-6,7,8,9-tetrahydropyrido[1,2-a]indol-10-yl]pyrazol-1-yl]-6-oxo-hexyl]carbamate (125 mg, 70%) as a yellow gum. ES/MS (m/z): (³⁵Cl/³⁷Cl) 804.4/806.4 (M+H) and 704.4/706.3 (M+H-Boc).

### EXAMPLE 196

### 6-Amino-N-[2-[[3,4-dichloro-10-(1H-pyrazol-4-yl)-6,7,8,9-tetrahydropyrido[1,2-a]indol-7-yl]amino]-2-oxo-ethyl]hexanamide formic acid salt

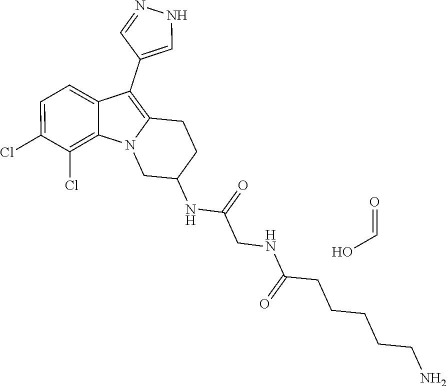

Dissolve tert-butyl N-[6-[4-[7-[[2-[6-(tert-butoxycarbonylamino)hexanoylamino]acetyl]amino]-3,4-dichloro-6,7,8,9-tetrahydropyrido[1,2-a]indol-10-yl]pyrazol-1-yl]-6-oxo-hexyl]carbamate (125 mg, 0.126 mmol) in DCM (2 mL) and add TFA (2 mL). Stir at ambient temperature for 1 hr. Concentrate under vacuum and dilute the residue with MeOH (2 mL). Add potassium carbonate (35 mg, 0.25 mmol). Stir at ambient temperature for 16-18 hrs. Concentrate under vacuum and dilute with water, extract with EtOAc, dry over anhydrous sodium sulfate, filter, and concentrate under vacuum. Purify by prep-HPLC to give 6-amino-N-[2-[[3,4-dichloro-10-(1H-pyrazol-4-yl)-6,7,8,9-tetrahydropyrido[1,2-a]indol-7-yl]amino]-2-oxo-ethyl]hexanamide formic acid salt (15.58 mg, 23%) as a white solid. ES/MS (m/z): (³⁵Cl/³⁷Cl) 491.4/493.4 (M+H).

### Intermediate 465

### 4-Benzyloxy-6,7-dichloro-1-(p-tolylsulfonyl)indole

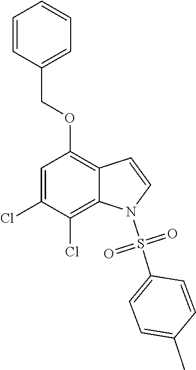 [SMILES: Cc1ccc(S(=O)(=O)n2ccc3c(OCc4ccccc4)cc(Cl)c(Cl)c32)cc1]

Dissolve 6,7-dichloro-1-(p-tolylsulfonyl)indol-4-ol (1.50 g, 3.79 mmol) in THF (15 mL). Add potassium carbonate (1.60 g, 11.6 mmol) and benzyl bromide (1.00 g, 5.85 mmol). Stir at 60° C. for 3 hrs. Dilute with water and extract with EtOAc, wash with saturated aqueous NaCl, dry over anhydrous sodium sulfate, filter, and concentrate under vacuum. Purify by flash column chromatography eluting with EtOAc in petroleum ether to give 4-benzyloxy-6,7-dichloro-1-(p-tolylsulfonyl)indole (1.85 g, 98%) as a black solid. ES/MS (m/z): (³⁵Cl/³⁷Cl) 445.9/447.8 (M+H).

### Intermediate 466

### 4-Benzyloxy-6,7-dichloro-1H-indole

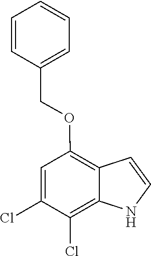 [SMILES: Clc1cc(OCc2ccccc2)c2cc[nH]c2c1Cl]

Dissolve 4-benzyloxy-6,7-dichloro-1-(p-tolylsulfonyl)indole (1.99 g, 4.01 mmol) in THF (20 mL) and add TBAF (10 mL, 1.0M in THF). Stir at ambient temperature for 16 hrs. Dilute with EtOAc, wash with saturated aqueous NH₄Cl and saturated aqueous NaCl, dry over anhydrous sodium sulfate, filter, and concentrate under vacuum. Purify by flash column chromatography eluting with EtOAc in petroleum ether to give 4-benzyloxy-6,7-dichloro-1H-indole (1.09 g, 91%) as a white solid.

### Intermediate 467

### 4-(Benzyloxy)-6,7-dichloro-1-((triisopropylsilyl)ethynyl)-1H-indole

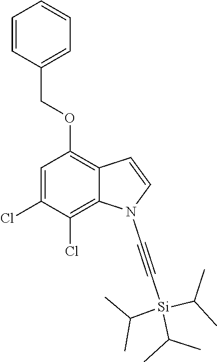 [SMILES: CC(C)[Si](C#Cn1ccc2c(OCc3ccccc3)cc(Cl)c(Cl)c21)(C(C)C)C(C)C]

Dissolve 4-benzyloxy-6,7-dichloro-1H-indole (900 mg, 3.02 mmol) in 1,4-dioxane (15 mL). Add (bromoethynyl)triisopropylsilane (4.10 g, 15.2 mmol), PEG-400 (300 mg), CuI (60 mg, 0.32 mmol) and Cs₂CO₃ (1.20 g, 3.68 mmol). Purge N₂ and stir at 130° C. for 16 hrs. Dilute with water and extract with EtOAc, wash with saturated aqueous NaCl, dry over anhydrous sodium sulfate, filter, and concentrate under vacuum. Purify by flash column chromatography eluting with EtOAc in petroleum ether to give 4-(benzyloxy)-6,7-dichloro-1-((triisopropylsilyl)ethynyl)-1H-indole (930 mg, 62%) as a yellow oil.

### Intermediate 468

### 4-Benzyloxy-6,7-dichloro-1-ethynyl-indole

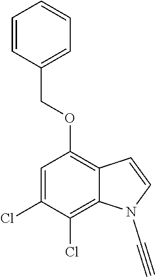 [SMILES: C#Cn1ccc2c(OCc3ccccc3)cc(Cl)c(Cl)c21]

Dissolve 4-(benzyloxy)-6,7-dichloro-1-((triisopropylsilyl)ethynyl)-1H-indole (930 mg, 1.87 mmol) in THF (20 mL) and add TBAF (10 mL, 1.0M in THF). Stir at ambient temperature for 2 hrs. Combine with another batch run at 0.05×scale for work up and purification. Dilute with water, extract with EtOAc (3×), wash the combined organic layers with saturated aqueous NaCl, dry over anhydrous Na₂SO₄, filter, and concentrate under vacuum. Purify by flash column chromatography eluting with EtOAc in petroleum ether to give 4-benzyloxy-6,7-dichloro-1-ethynyl-indole (700 mg, 99+%) as a yellow solid.

### Intermediate 469

### 2-[4-(4-Benzyloxy-6,7-dichloro-indol-1-yl)triazol-1-yl]ethoxy-tert-butyl-dimethyl-silane

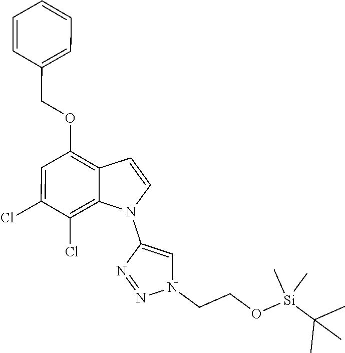 [SMILES: CC(C)(C)[Si](C)(C)OCCn1cc(-n2ccc3c(OCc4ccccc4)cc(Cl)c(Cl)c32)nn1]

Dissolve 4-benzyloxy-6,7-dichloro-1-ethynyl-indole (630 mg, 1.89 mmol) in DMF (5 mL) and water (5 mL). Add copper (25 mg, 0.39 mmol), cupric sulfate (61 mg, 0.38 mmol) and 2-azidoethoxy-tert-butyl-dimethyl-silane (490 mg, 2.31 mmol). Purge with N₂ and stir at 110° C. for 0.5 hr. Dilute with water and extract with EtOAc (3×). Dry combined organic layers over anhydrous sodium sulfate, filter, and concentrate under vacuum. Purify by flash column chromatography eluting with EtOAc in petroleum ether to give 2-[4-(4-benzyloxy-6,7-dichloro-indol-1-yl)triazol-1-yl]ethoxy-tert-butyl-dimethyl-silane (670 mg, 65%) as a black oil. ES/MS (m/z): (³⁵Cl/³⁷Cl) 517.0/519.0 (M+H).

### Intermediate 470

### 1-[1-[2-[tert-Butyl(dimethyl)silyl]oxyethyl]triazol-4-yl]-6,7-dichloro-indol-4-ol

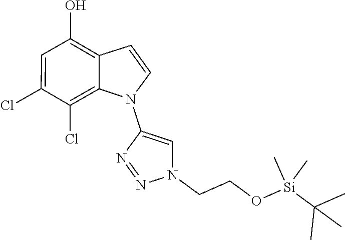 [SMILES: CC(C)(C)[Si](C)(C)OCCn1cc(-n2ccc3c(O)cc(Cl)c(Cl)c32)nn1]

Dissolve 2-[4-(4-benzyloxy-6,7-dichloro-indol-1-yl)triazol-1-yl]ethoxy-tert-butyl-dimethyl-silane (560 mg, 1.06 mmol) in methanol (15 mL) and add 10% Pd/C (60 mg). Purge with H₂ and stir at ambient temperature for 16 hrs under H₂ (15 psi). Filter and concentrate under vacuum. Purify by flash column chromatography eluting with EtOAc in petroleum ether to give 1-[1-[2-[tert-butyl(dimethyl)silyl]oxyethyl]triazol-4-yl]-6,7-dichloro-indol-4-ol (468 mg, 93%) as a white solid. ES/MS (m/z): (³⁵Cl/³⁷Cl) 427.2/429.1 (M+H).

### Intermediate 471

### 2-[1-[1-[2-[tert-Butyl(dimethyl)silyl]oxyethyl]triazol -4-yl]-6,7-dichloro-indol-4-yl]oxyacetonitrile

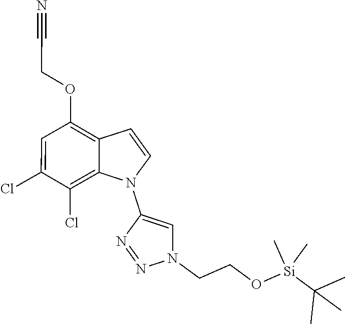 [SMILES: CC(C)(C)[Si](C)(C)OCCn1cc(-n2ccc3c(OCC#N)cc(Cl)c(Cl)c32)nn1]

Dissolve 1-[1-[2-[tert-butyl(dimethyl)silyl]oxyethyl]triazol-4-yl]-6,7-dichloro-indol-4-ol (410 mg, 0.940 mmol) in DMF (6 mL). Add bromoacetonitrile (240 mg, 1.94 mmol) and potassium carbonate (130 mg, 0.941 mmol). Stir at ambient temperature for 16 hrs. Combine with another batch run at 0.12×scale for work up and purification. Dilute with EtOAc, and wash with water. Extract the aqueous layer with EtOAc (3×). Wash the combined organic layers with saturated aqueous NaCl, dry over sodium sulfate, filter, and concentrate under vacuum. Purify by flash column chromatography eluting with EtOAc in petroleum ether to give 2-[1-[1-[2-[ter t-butyl(dimethyl)silyl]oxyethyl]triazol-4-yl]-6,7-dichloro-indol-4-yl]oxyacetonitrile (500 mg, 97%) as a colorless oil. ES/MS (m/z): (³⁵Cl/³⁷Cl) 466.2/468.1 (M+H).

### Intermediate 472

### 2-((3-Bromo-6,7-dichloro-1-(1-(2-hydroxyethyl)-1H-1,2,3-triazol-4-yl)-1H-indol-4-yl)oxy)acetonitrile

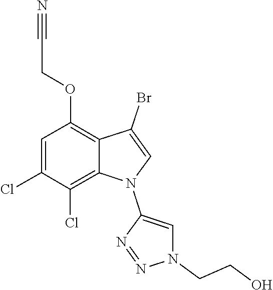 [SMILES: N#CCOc1cc(Cl)c(Cl)c2c1c(Br)cn2-c1cn(CCO)nn1]

Dissolve 2-[1-[1-[2-[tert-butyl(dimethyl)silyl]oxyethyl]triazol-4-yl]-6,7-dichloro-indol-4-yl]oxyacetonitrile (390 mg, 0.794 mmol) in DMF (5 mL) and add NBS (145 mg, 0.815 mmol) at 0° C. Stir at ambient temperature for 2 hrs. Dilute with water, extract with EtOAc (3×), wash the combined organic layers with aqueous Na₂SO₃ (3×), dry over anhydrous sodium sulfate, filter, and concentrate under vacuum. Purify by flash column chromatography eluting with EtOAc in petroleum ether to give 2-((3-bromo-6,7-dichloro-1-(1-(2-hydroxyethyl)-1H-1,2,3-triazol-4-yl)-1H-indol-4-yl)oxy)acetonitrile (230 mg, 64%) as a yellow oil. ES/MS (m/z): 430.0/431.9/433.9 (M+H).

### Intermediate 473

### 2-((6,7-Dichloro-1-(1-(2-hydroxy ethyl)-1H-1,2,3-triazol-4-yl)-3-(1-(tetrahydro-2H-pyran-2-yl)-1H-pyrazol-4-yl)-1H-indol-4-yl)oxy)acetonitrile

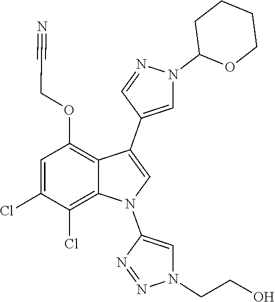 [SMILES: N#CCOc1cc(Cl)c(Cl)c2c1c(-c1cnn(C3CCCCO3)c1)cn2-c1cn(CCO)nn1]

Dissolve 2-((3-bromo-6,7-dichloro-1-(1-(2-hydroxyethyl)-1H-1,2,3-triazol-4-yl)-1H-indol-4-yl)oxy)acetonitrile (230 mg, 0.507 mmol), 1-(tetrahydro-2H-pyran-2-yl)-4-(4,4,5,5-tetramethyl-1,3,2-dioxaborolan-2-yl)-1H-pyrazole (230 mg, 0.810 mmol), Pd(dppf)Cl₂ (45 mg, 0.058 mmol), and K₂CO₃ (150 mg, 1.09 mmol) in 1,4-dioxane (8 mL) and water (2 mL). Purge with N₂ and stir at 90° C. for 2 hrs. Dilute with water and extract with EtOAc, wash with saturated aqueous NaCl, dry over anhydrous sodium sulfate, filter, and concentrate under vacuum. Purify by flash column chromatography eluting with EtOAc in petroleum ether to give 2-((6,7-dichloro-1-(1-(2-hydroxyethyl)-1H-1,2,3-triazol-4-yl)-3-(1-(tetrahydro-2H-pyran-2-yl)-1H-pyrazol-4-yl)-1H-indol-4-yl)oxy)acetonitrile (170 mg, 63%) as a yellow oil. ES/MS (m/z): (³⁵Cl/³⁷Cl) 502.1/504.0 (M+H).

### EXAMPLE 197

### 22-[6, 7-Dichloro-1-[1-(2-hydroxy ethyl)triazol-4-yl]-3-(1H-pyrazol-4-yl)indol-4-yl]oxyacetonitrile

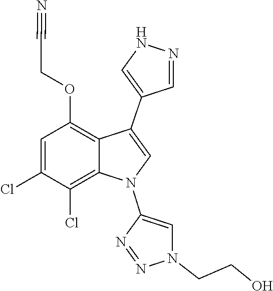 [SMILES: N#CCOc1cc(Cl)c(Cl)c2c1c(-c1cn[nH]c1)cn2-c1cn(CCO)nn1]

Suspend 2-((6,7-dichloro-1-(1-(2-hydroxyethyl)-1H-1,2,3-triazol -4-yl)-3-(1-(tetrahydro-2H-pyran-2-yl)-1H-pyrazol-4-yl)-1H-indol-4-yl)oxy)acetonitrile (170 mg, 0.322 mmol) in DCM (3 mL). Add anisole (350 mg, 3.24 mmol) and TFA (3 mL). Stir at ambient temperature for 2 hrs. Concentrate under vacuum and dissolve the residue in THF (1 mL) and add LiOH (5 mg, 0.21 mmol) in water (0.5 mL), then stir at ambient temperature for 2 hrs. Concentrate under vacuum and purify by prep-HPLC to give 2-[6,7-dichloro-1-[1-(2-hydroxyethyl)triazol-4-yl]-3-(1H-pyrazol-4-yl)indol-4-yl]oxyacetonitrile (71.5 mg, 52%) as a white solid. ES/MS (m/z): (³⁵Cl/³⁷Cl) 418.3/420.3 (M+H).

### Intermediate 474

### tert-Butyl N-[6,7-dichloro-3-(1-tetrahydropyran-2-ylpyrazol-4-yl)-1-(2-triisopropylsilylethynyl)indol-4-yl]carbamate

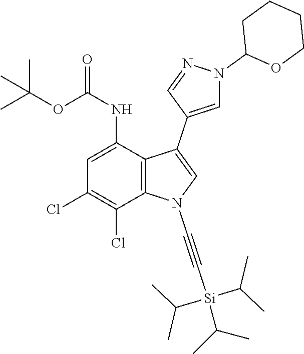 [SMILES: CC(C)[Si](C#Cn1cc(-c2cnn(C3CCCCO3)c2)c2c(NC(=O)OC(C)(C)C)cc(Cl)c(Cl)c21)(C(C)C)C(C)C]

Dissolve tert-butyl N-[6,7-dichloro-3-(1-tetrahydropyran-2-ylpyrazol-4-yl)-1H-indol-4-yl]carba mate (2.00 g, 4.17 mmol) in 1,4-dioxane (40 mL). Add (bromoethynyl)triisopropylsilane (5.61 g, 20.8 mmol), PEG-400 (600 mg), CuI (80 mg, 0.42 mmol) and Cs₂CO₃ (1.63 g, 5.00 mmol). Purge with N₂ and stir at 130° C. for 16 hrs. Dilute with water, extract with EtOAc, wash with saturated aqueous NaCl, dry over anhydrous sodium sulfate, filter, and concentrate under vacuum. Purify by flash column chromatography eluting with EtOAc in petroleum ether to give tert-butyl N-[6,7-dichloro-3-(1-tetrahydropyran-2-ylpyrazol-4-yl)-1-(2-triisopropylsilylethynyl)indol-4-yl]carbamate (1.50 g, 54%) as a yellow oil.

### Intermediate 475

### tert-Butyl N-[6,7-dichloro-1-ethynyl-3-(1-tetrahydropyran-2-ylpyrazol-4-yl)indol-4-yl]carbamate

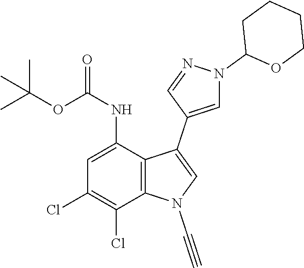 [SMILES: C#Cn1cc(-c2cnn(C3CCCCO3)c2)c2c(NC(=O)OC(C)(C)C)cc(Cl)c(Cl)c21]

Dissolve tert-butyl N-[6,7-dichloro-3-(1-tetrahydropyran-2-ylpyrazol-4-yl)-1-(2-triisopropylsilylethynyl)indol-4-yl]carbamate (1.5 g, 2.3 mmol) in THF (20 mL) and add TBAF (10 mL, 1.0M in THF). Stir at ambient temperature for 2 hrs. Combine with another batch run at 0.19×scale for work up and purification. Dilute with EtOAc, wash with saturated aqueous NH₄Cl and saturated aqueous NaCl, dry over anhydrous sodium sulfate, filter, and concentrate under vacuum. Purify by flash column chromatography eluting with EtOAc in petroleum ether to give tert-butyl N-[6,7-dichloro-1-ethynyl-3-(1-tetrahydropyran-2-ylpyrazol-4-yl)indol-4-yl]carbamate (1.29 g, 97%) as a brown solid. ES/MS (m/z): (³⁵Cl/³⁷Cl) 474.9/476.9 (M+H).

### Intermediate 476

### tert-Butyl N-[1-[1-[2-[tert-butyl(dimethyl)silyl]oxyethyl]triazol-4-yl]-6,7-dichloro-3-(1-tetrahydropyran-2-ylpyrazol-4-yl)indol-4-yl]carbamate

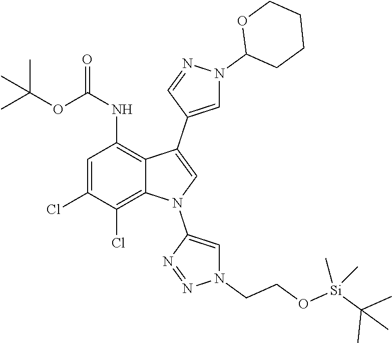 [SMILES: CC(C)(C)OC(=O)Nc1cc(Cl)c(Cl)c2c1c(-c1cnn(C3CCCCO3)c1)cn2-c1cn(CCO[Si](C)(C)C(C)(C)C)nn1]

Dissolve tert-butyl N-[6, 7-dichloro-1-ethynyl-3-(1-tetrahydropyran-2-ylpyrazol -4-yl)indol-4-yl]carbamate (500 mg, 1.03 mmol) in DMF (5 mL) and water (5 mL). Add copper (15 mg, 0.24 mmol), cupric sulfate (35 mg, 0.22 mmol) and 2-azidoethoxy-tert-butyl-dimethyl-silane (270 mg, 1.27 mmol). Purge with N₂ and stir at 110° C. for 0.5 hr. Dilute with water, extract with EtOAc, dry over anhydrous sodium sulfate, filter, and concentrate under vacuum. Purify by flash column chromatography eluting with MeOH in DCM to give tert-butyl N-[1-[1-[2-[tert-butyl(dimethyl)silyl]oxyethyl]triazol-4-yl]-6,7-dichloro-3-(1-tetrahydropyran-2-ylpyrazol-4-yl)indol-4-yl]carbamate (740 mg, 81%) as a black oil. ES/MS (m/z): (³⁵Cl/³⁷Cl) 676.4/678.3 (M+H).

### Intermediate 477

### 2-[4-[4-Amino-6,7-dichloro-3-(1H-pyrazol-4-yl)indol-1-yl]triazol-1-yl]ethanol hydrochloride

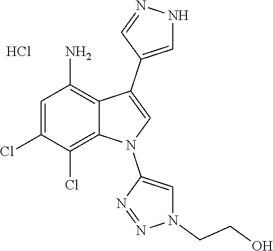 [SMILES: Cl ; Nc1cc(Cl)c(Cl)c2c1c(-c1cn[nH]c1)cn2-c1cn(CCO)nn1]

Dissolve tert-butyl N-[1-[1-[2-[tert-butyl(dimethyl)silyl]oxyethyl]triazol-4-yl]-6,7-dichloro-3-(1-tetrahydropyran-2-ylpyrazol-4-yl)indol-4-yl]carbamate (740 mg, 1.09 mmol) in 4M HCl in MeOH (10 mL). Stir at ambient temperature for 2 hrs., and concentrate under vacuum to give 2-[4-[4-amino-6,7-dichloro-3-(1H-pyrazol-4-yl)indol-1-yl]triazol-1-yl]ethanol hydrochloride (438 mg, 89%) as a yellow solid. ES/MS (m/z): (³⁵Cl/³⁷Cl) 378.0/379.9 (M+H).

### Intermediate 478

### 2-[4-[4-[3-[tert-Butyl(dimethyl)silyl]oxypropylamino]-6,7-dichloro-3-(1H-pyrazol-4-yl)indol-1-yl]triazol-1-yl]ethanol

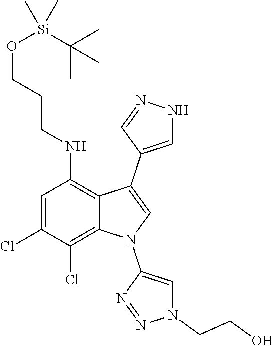 [SMILES: CC(C)(C)[Si](C)(C)OCCCNc1cc(Cl)c(Cl)c2c1c(-c1cn[nH]c1)cn2-c1cn(CCO)nn1]

Dissolve 2-[4-[4-amino-6,7-dichloro-3-(1H-pyrazol-4-yl)indol-1-yl]triazol-1-yl]ethanol (100 mg, 0.222 mmol, HCl salt) in MeOH (5 mL) and add DIPEA (90 mg, 0.70 mmol) to adjust to pH=7. Stir at ambient temperature for 0.5 hr. Add acetic acid (15 mg, 0.25 mmol) to adjust to pH=5 and stir at ambient temperature for 0.5 hr. Add 3-[(tert-butyldimethylsilyl)oxy]-1-propanal (140 mg, 0.706 mmol) and stir at ambient temperature for 0.5 hr. Add sodium cyanoborohydride (45 mg, 0.68 mmol) and stir at ambient temperature for 16 hrs. Combine with another batch run at 0.50×scale for work up and purification. Dilute with water and adjust to pH=8 with saturated aqueous NaHCO₃. Extract with EtOAc (2×). Wash the combined organic layers with saturated aqueous NaCl, dry over anhydrous sodium sulfate, filter, and concentrate under vacuum to give 2-[4-[4-[3-[tert-butyl(dimethyl)silyl]oxypropylamino]-6,7-dichloro-3-(1H-pyrazol-4-yl)indol-1-yl]triazol-1-yl]ethanol (200 mg, 65%) as a brown oil.

### EXAMPLE 198

### 3-[[6,7-Dichloro-1-[1-(2-hydroxyethyl)triazol-4-yl]-3-(1H-pyrazol-4-yl)indol-4-yl]amino]propan-1-ol

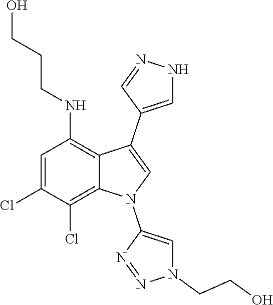 [SMILES: OCCCNc1cc(Cl)c(Cl)c2c1c(-c1cn[nH]c1)cn2-c1cn(CCO)nn1]

Suspend 2-[4-[4-[3-[tert-butyl(dimethyl)silyl]oxypropylamino]-6,7-dichloro-3-(1H-pyrazol-4-yl)indol-1-yl]triazol-1-yl]ethanol (200 mg, 0.218 mmol) in THF (6 mL) and add 6M aqueous HCl (3 mL). Stir at ambient temperature for 1 hr. and concentrate under vacuum. Purify by prep-HPLC to give 3-[[6,7-dichloro-1-[1-(2-hydroxyethyl)triazol-4-yl]-3-(1H-pyrazol-4-yl)indol-4-yl]amino]propan-1-ol (35.0 mg, 36%) as a white solid. ES/MS (m/z): (³⁵Cl/³⁷Cl) 436.3/438.3 (M+H).

### EXAMPLE 199

### N-[6,7-Dichloro-1-[1-(2-hydroxy ethyl)triazol-4-yl]-3-(1H-pyrazol-4-yl)indol-4-yl]acetamide

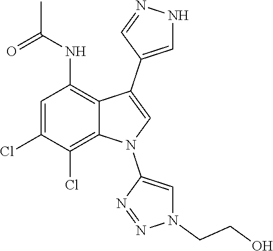 [SMILES: CC(=O)Nc1cc(Cl)c(Cl)c2c1c(-c1cn[nH]c1)cn2-c1cn(CCO)nn1]

Suspend 2-[4-[4-amino-6,7-dichloro-3-(1H-pyrazol-4-yl)indol-1-yl]triazol yl]ethanol (100 mg, 0.243 mmol) in DCM (8 mL) and add TEA (80 mg, 0.79 mmol). Add acetyl chloride (30 mg, 0.38 mmol) and stir at ambient temperature for 2 hrs. Quench with saturated aqueous NaHCO₃ and extract with EtOAc, wash with water, saturated aqueous NaCl, dry over anhydrous Na₂SO₄, filter, and concentrate under vacuum. Dissolve the residue in MeOH (5 mL) and add potassium carbonate (101 mg, 0.731 mmol). Stir at ambient temperature for 3 hrs. Dilute with water and extract with EtOAc, wash with water, saturated aqueous NaCl, dry over anhydrous Na₂SO₄, filter, and concentrate under vacuum. Purify by prep-HPLC to give N-[6,7-dichloro-1-[1-(2-hydroxyethyl)triazol-4-yl]-3-(1H-pyrazol-4-yl)indol-4-yl]acetamide (39.6 mg, 39%) as a white solid. ES/MS (m/z): (³⁵Cl/³⁷Cl) 420.3/422.3 (M+H).

### Intermediate 479

### 4-Bromo-6-chloro-1-tosyl-1H-indole

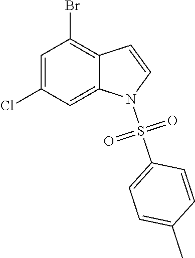 [SMILES: Cc1ccc(S(=O)(=O)n2ccc3c(Br)cc(Cl)cc32)cc1]

Dissolve 4-bromo-6-chloro-1H-indole (2.0 g, 8.4 mmol) in THF (20 mL) and add sodium hydride (1.4 g, 35 mmol, 60% dispersion in mineral oil) slowly. Stir at 0° C. for 0.5 hr. Add 4-methylbenzenesulfonyl chloride (3.2 g, 17 mmol) at 0° C. and stir at ambient temperature for 16 hrs. Quench at 0° C. with saturated aqueous NH₄Cl and water. Filter and extract the filtrate with EtOAc, dry over anhydrous sodium sulfate, filter, concentrate under vacuum, and combine with the filter cake. Purify by trituration with PE/EtOAc (10/1) to give 4-bromo-6-chloro-1-tosyl-1H-indole (3.80 g, 99%) as a white solid. ES/MS (m/z): 384.0/385.8/387.9 (M+H).

### Intermediate 480

### tert-Butyl (6-chloro-1-tosyl-1H-indol-4-yl)carbamate

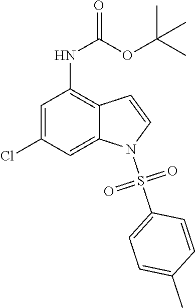 [SMILES: Cc1ccc(S(=O)(=O)n2ccc3c(NC(=O)OC(C)(C)C)cc(Cl)cc32)cc1]

Suspend 4-bromo-6-chloro-1-tosyl-1H-indole (3.6 g, 8.4 mmol), tert-butyl carbamate (2.2 g, 19 mmol), Pd₂(dba)₃ (₈60 mg, 0.939 mmol), Xantphos (1.1 g, 1.9 mmol) and Cs₂CO₃ (7.6 g, 23 mmol) in 1,4-dioxane (50 mL). Purge with N₂ then stir at 100° C. for 3 hrs. Cool to ambient temperature and filter. Dilute the filtrate with water and extract with EtOAc (2×). Dry the combined organic layers over anhydrous sodium sulfate, filter, and concentrate under vacuum. Purify by trituration with PE/EtOAc (5/1) to give tert-butyl (6-chloro-1-tosyl-1H-indol-4-yl)carbamate (4.28 g, 97%) as a yellow solid. ES/MS (m/z): (³⁵Cl/³⁷Cl) 365.1/367.0 (M+H-tBu).

### Intermediate 481

### tert-Butyl N-(6-chloro-1H-indol-4-yl)carbamate

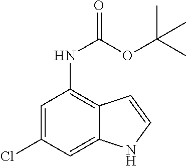 [SMILES: CC(C)(C)OC(=O)Nc1cc(Cl)cc2[nH]ccc12]

Dissolve tert-butyl (6-chloro-1-tosyl-1H-indol-4-yl)carbamate (1.0 g, 1.9 mmol) in water (5 mL) and MeOH (20 mL) and add sodium hydroxide (1.00 g, 25.0 mmol). Stir at 60° C. for 16 hrs. Cool to ambient temperature, concentrate under vacuum, dilute with water, and extract with EtOAc (2×). Wash the combined organic layers with saturated aqueous NaCl, dry over sodium sulfate, filter, and concentrate under vacuum. Purify by flash column chromatography eluting with EtOAc in petroleum ether to give tert-butyl N-(6-chloro-1H-indol-4-yl)carbamate (330 mg, 57%) as a yellow oil. ES/MS (m/z): (³⁵Cl/³⁷Cl) 211.2/213.1 (M+H-tBu).

### Intermediate 482

### tert-Butyl N-(6-chloro-3-iodo-1H-indol-4-yl)carbamate

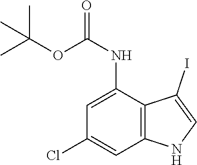 [SMILES: CC(C)(C)OC(=O)Nc1cc(Cl)cc2[nH]cc(I)c12]

Dissolve tert-butyl N-(6-chloro-1H-indol-4-yl)carbamate (330 mg, 1.08 mmol) in DMF (40 mL) and add NIS (270 mg, 1.18 mmol) at 0° C. Stir at 0° C. for 2 hrs. Dilute with EtOAc, wash with saturated aqueous Na₂SO₃ and saturated aqueous NaCl, dry over anhydrous sodium sulfate, filter, and concentrate under vacuum. Purify by flash column chromatography eluting with EtOAc in petroleum ether to give tert-butyl N-(6-chloro-3-iodo-1H-indol-4-yl)carbamate (400 mg, 80%) as a brown solid. ES/MS (m/z): (³⁵Cl/³⁷Cl) 210.1/212.1 (M+H-tBu-I).

### Intermediate 483

### tert-Butyl N-[6-chloro-3-(1-tetrahydropyran-2-ylpyrazol-4-yl)-1H-indol-4-yl]carbamate

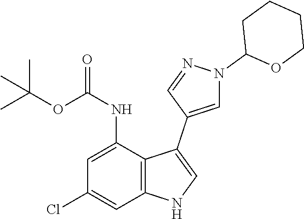 [SMILES: CC(C)(C)OC(=O)Nc1cc(Cl)cc2[nH]cc(-c3cnn(C4CCCCO4)c3)c12]

Suspend tert-butyl N-(6-chloro-3-iodo-1H-indol-4-yl)carbamate (1.42 g, 3.26 mmol), 1-(tetrahydro-2H-pyran-2-yl)-4-(4,4,5,5-tetramethyl-1,3,2-dioxaborolan-2-yl)-1H-pyrazole (2.8 g, 9.9 mmol), Na₂CO₃ (1.05 g, 9.86 mmol) and Pd(dtbpf)Cl₂ (435 mg, 0.654 mmol) in 1,4-dioxane (24 mL) and water (6 mL). Purge with N₂ and stir at 90° C. for 2 hrs. Filter and concentrate the filtrate. Purify by flash column chromatography eluting with EtOAc in petroleum ether to give tert-butyl N-[6-chloro-3-(1-tetrahydropyran-2-ylpyrazol-4-yl)-1H-indol-4-yl]carbamate (1.24 g, 87%) as a brown solid. ES/MS (m/z): (³⁵Cl/³⁷Cl) 417.2/419.2 (M+H).

### Intermediate 484

### tert-Butyl N-[6-chloro-3-(1-tetrahydropyran-2-ylpyrazol-4-yl)-1-(2-triisopropylsilylethynyl)indol-4-yl]carbamate

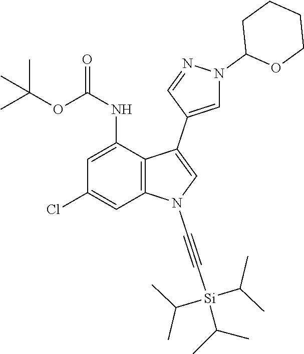 [SMILES: CC(C)[Si](C#Cn1cc(-c2cnn(C3CCCCO3)c2)c2c(NC(=O)OC(C)(C)C)cc(Cl)cc21)(C(C)C)C(C)C]

Dissolve tert-butyl N-[6-chloro-3-(1-tetrahydropyran-2-ylpyrazol-4-yl)-1H-indol-4-yl]carbamate (760 mg, 1.55 mmol) in 1,4-dioxane (15 mL). Add (bromoethynyl)triisopropylsilane (2.10 g, 7.80 mmol), PEG-400 (200 mg), CuI (30 mg, 0.16 mmol) and Cs₂CO₃ (610 mg, 1.87 mmol). Purge with N₂. Stir at 130° C. for 16 hrs. Dilute with water and extract with EtOAc, wash with saturated aqueous NaCl, dry over anhydrous sodium sulfate, filter, and concentrate under vacuum. Purify by flash column chromatography eluting with EtOAc in petroleum ether to give tert-butyl N-[6-chloro-3-(1-tetrahydropyran-2-ylpyrazol-4-yl)-1-(2-triisopropylsilylethynyl)indol-4-yl]carbamate (320 mg, 33%) as a yellow oil.

### Intermediate 485

### tert-Butyl (6-chloro-1-ethynyl-3-(1-(tetrahydro-2H-pyran-2-yl)-1H-pyrazol-4-yl)-1H-indol-4-yl)carbamate

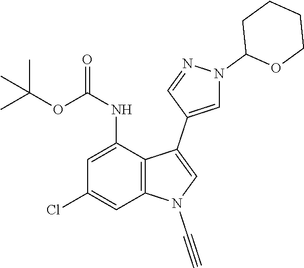 [SMILES: C#Cn1cc(-c2cnn(C3CCCCO3)c2)c2c(NC(=O)OC(C)(C)C)cc(Cl)cc21]

Dissolve tert-butyl N-[6-chloro-3-(1-tetrahydropyran-2-ylpyrazol-4-yl)-1-(2-triisopropylsilylethynyl)indol-4-yl]carbamate (400 mg, 0.636 mmol) in THF (10 mL) and add TBAF (5 mL, 1.0M in THF). Stir at ambient temperature for 3 hrs. Dilute with EtOAc, wash with saturated aqueous NH₄Cl, dry over anhydrous sodium sulfate, filter, and concentrate under vacuum. Purify by flash column chromatography eluting with EtOAc in petroleum ether to give tert-butyl (6-chloro-1-ethynyl-3-(1-(tetrahydro-2H-pyran-2-yl)-1H-pyrazol-4-yl)-1H-indol-4-yl)carbamate (250 mg, 85%) as a yellow solid. ES/MS (m/z): (³⁵Cl/³⁷Cl) 441.2/443.1 (M+H) and 301.1/303.1 (M+H-tBu-THP).

### Intermediate 486

### tert-Butyl N-[1-[1-[2-[tert-butyl(dimethyl)silyl]oxyethyl]triazol-4-yl]-6-chloro-3-(1-tetrahydropyran-2-ylpyrazol-4-yl)indol-4-yl]carbamate

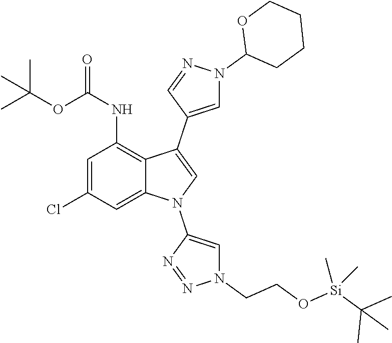 [SMILES: CC(C)(C)OC(=O)Nc1cc(Cl)cc2c1c(-c1cnn(C3CCCCO3)c1)cn2-c1cn(CCO[Si](C)(C)C(C)(C)C)nn1]

Dissolve tert-butyl (6-chloro-1-ethynyl-3-(1-(tetrahydro-2H-pyran-2-yl)-1H-pyrazol-4-yl)-1H-indol-4-yl)carbamate (250 mg, 0.539 mmol) in DMF (5 mL) and water (5 mL). Add copper (10 mg, 0.16 mmol), cupric sulfate (20 mg, 0.13 mmol) and 2-azidoethoxy-tert-butyl-dimethyl-silane (140 mg, 0.661 mmol). Purge with N₂ and stir at 110° C. for 0.5 hr. Dilute with water, extract with EtOAc, wash with saturated aqueous NaCl, dry over anhydrous sodium sulfate, filter, and concentrate under vacuum. Purify by flash column chromatography eluting with EtOAc in petroleum ether to give tert-butyl N-[1-[1-[2-[tert-butyl(dimethyl)silyl]oxyethyl]triazol-4-yl]-6-chloro-3-(1-tetrahydropyran-2-ylpyrazol-4-yl)indol-4-yl]carbamate (340 mg, 88%) as a white solid. ES/MS (m/z): (³⁵Cl/³⁷Cl) 642.4/644.2 (M+H) and 502.2/504.2 (M+H-tBu-THP).

### Intermediate 487

### 2-[4-[4-Amino-6-chloro-3-(1H-pyrazol-4-yl)indol-1-yl]triazol-1-yl]ethanol hydrochloride

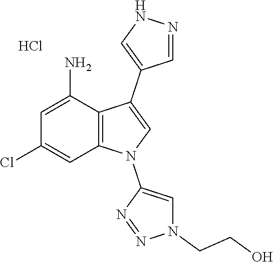 [SMILES: Nc1cc(Cl)cc2c1c(-c1cn[nH]c1)cn2-c1cn(CCO)nn1 ; Cl]

Dissolve tert-butyl N-[1-[1-[2-[tert-butyl(dimethyl)silyl]oxyethyl]triazol-4-yl]-6-chloro-3-(1-tetrahydropyran-2-ylpyrazol-4-yl)indol-4-yl]carbamate (340 mg, 0.476 mmol) in 4M HCl in MeOH (10 mL). Stir at ambient temperature for 2 hrs., and concentrate under vacuum to give 2-[4-[4-amino-6-chloro-3-(1H-pyrazol-4-yl)indol-1-yl]triazol-1-yl]ethanol hydrochloride (180 mg, 89%) as a yellow solid. ES/MS (m/z): (³⁵Cl/³⁷Cl) 344.1/346.1 (M+H).

### Intermediate 488

### 2-[4-[4-[3-[tert-Butyl(dimethyl)silyl]oxypropylamino]-6-chloro-3-(1H-pyrazol-4-yl)indol-1-yl]triazol-1-yl]ethanol

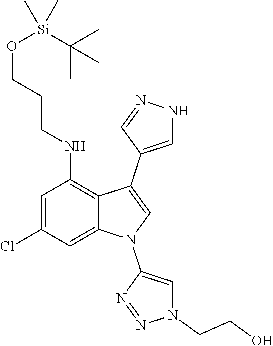 [SMILES: CC(C)(C)[Si](C)(C)OCCCNc1cc(Cl)cc2c1c(-c1cn[nH]c1)cn2-c1cn(CCO)nn1]

Dissolve 2-[4-[4-amino-6-chloro-3-(1H-pyrazol-4-yl)indol-1-yl]triazol-1-yl]ethanol hydrochloride (80 mg, 0.21 mmol) in MeOH (5 mL) and add DIPEA to adjust pH=7. Stir at ambient temperature for 0.5 hr. Add acetic acid to adjust to pH=5 and stir at ambient temperature for 0.5 hr. Add 3-[(tert-butyldimethylsilyl)oxy]-1-propanal (85 mg, 0.43 mmol) and stir at ambient temperature for 0.5 hr. Add sodium cyanoborohydride (42 mg, 0.64 mmol) and stir at ambient temperature for 16 hrs. Combine with another batch run at 0.25×scale for work up and purification. Dilute with water and adjust to pH=8 with saturated aqueous NaHCO₃, extract with EtOAc, wash with saturated aqueous NaCl, dry over anhydrous sodium sulfate, filter, and concentrate under vacuum. Purify by flash column chromatography eluting with MeOH in DCM to give 2-[4-[4-[3-[tert-butyl(dimethyl)silyl]oxypropylamino]-6-chloro-3-(1H-pyrazol-4-yl)indol-1-yl]triazol-1-yl]ethanol (140 mg, 78%) as a brown oil. ES/MS (m/z): (³⁵Cl/³⁷Cl) 516.3/518.2 (M+H).

### EXAMPLE 200

### 3-[[6-Chloro-1-[1-(2-hydroxyethyl)triazol-4-yl]-3-(1H-pyrazol-4-yl)indol-4-yl]amino]propan-1-ol

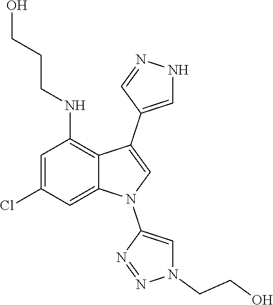 [SMILES: OCCCNc1cc(Cl)cc2c1c(-c1cn[nH]c1)cn2-c1cn(CCO)nn1]

Suspend 2-[4-[4-[3-[tert-butyl(dimethyl)silyl]oxypropylamino]-6-chloro-3-(1H-pyrazol-4-yl)indol-1-yl]triazol-1-yl]ethanol (140 mg, 0.206 mmol) in THF (6 mL) and add 6M aqueous (3 mL). Stir at ambient temperature for 2 hrs. and concentrate under vacuum. Purify by prep-HPLC to give 3-[[6-chloro-1-[1-(2-hydroxyethyl)triazol-4-yl]-3-(1H-pyrazol-4-yl)indol-4-yl]amino]propan-1-ol (56.7 mg, 68%) as a white solid. ES/MS (m/z): (³⁵Cl/³⁷Cl) 402.3/404.3 (M+H).

### EXAMPLE 201

### N-[6-Chloro-1-[1-(2-hydroxyethyl)triazol-4-yl]-3-(1H-pyrazol-4-yl)indol-4-yl]acetamide

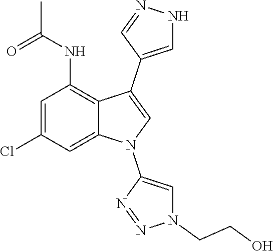 [SMILES: CC(=O)Nc1cc(Cl)cc2c1c(-c1cn[nH]c1)cn2-c1cn(CCO)nn1]

Suspend 2-[4-[4-amino-6-chloro-3-(1H-pyrazol-4-yl)indol-1-yl]triazol-1-yl]ethanol (120 mg, 0.349 mmol), TEA (220 mg, 2.17 mmol) and DMAP (5 mg, 0.04 mmol) in DCM (6 mL). Add acetyl chloride (0.25 mL, 3.51 mmol) and stir at ambient temperature for 2 hrs. Combine with another batch run at 0.17×scale for work up and purification. Concentrate under vacuum and dissolve the residue in MeOH (10 mL). Add potassium carbonate (150 mg, 1.09 mmol) and stir at ambient temperature for 16 hrs. Filter and concentrate the filtrate under vacuum. Purify by prep-HPLC to give N-[6-chloro-1-[1-(2-hydroxyethyl)triazol-4-yl]-3-(1H-pyrazol-4-yl)indol-4-yl]acetamide (46.5 mg, 30%) as a white solid. ES/MS (m/z): (³⁵Cl/³⁷Cl) 386.3/388.3 (M+H).

### Intermediate 489

### Ethyl 4-bromo-6, 7-dichloro-1-(4-ethoxy-4-oxo-butyl)indole-2-carboxylate

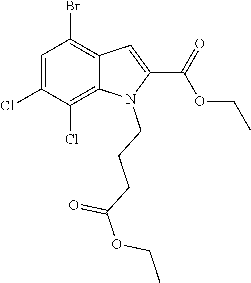 [SMILES: CCOC(=O)CCCn1c(C(=O)OCC)cc2c(Br)cc(Cl)c(Cl)c21]

Dissolve ethyl 4-bromo-6,7-dichloro-1H-indole-2-carboxylate (4.50 g, 12.0 mmol) in DMF (50 mL) and add Cs₂CO₃ (7.85 g, 24.1 mmol), then stir at ambient temperature for 1 hr. Add ethyl 4-bromobutyrate (2.75 mL, 18.3 mmol). Stir at 50° C. for 16-18 hrs. Combine with another batch run at 0.11×scale for work up and purification. Quench with 1M aqueous HCl at 0° C., extract with EtOAc, wash with saturated aqueous NaCl, dry over anhydrous sodium sulfate, filter, and concentrate under vacuum. Purify by flash column chromatography eluting with EtOAc in petroleum ether to give ethyl 4-bromo-6,7-dichloro-1-(4-ethoxy-4-oxo-butyl)indole-2-carboxylate (4.60 g, 73%) as a light-yellow solid.

### Intermediate 490

### Ethyl 1-bromo-3,4-dichloro-9-oxo-7,8-dihydro-6H-pyrido[1,2-a]indole-8-carboxylate

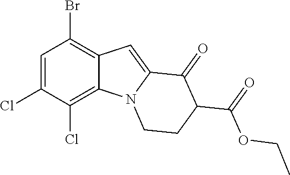 [SMILES: CCOC(=O)C1CCn2c(cc3c(Br)cc(Cl)c(Cl)c32)C1=O]

Suspend ethyl 4-bromo-6,7-dichloro-1-(4-ethoxy-4-oxo-butyl)indole-2-carboxylate (4.60 g, 9.69 mmol) in THF (50 mL). Add t-BuOK (2.30 g, 19.9 mmol). Stir at ambient temperature for 16-18 hrs. Quench with 1M aqueous HCl, extract with EtOAc, wash with saturated aqueous NaCl, dry over anhydrous sodium sulfate, filter, and concentrate under vacuum to give ethyl 1-bromo-3,4-dichloro-9-oxo-7,8-dihydro-6H-pyrido[1,2-a]indole-8-carboxylate (4.10 g, 94%) as a yellow solid.

### Intermediate 491

### 1-Bromo-3,4-dichloro-7, 8-dihydro-6H-pyrido[1,2-a]indol-9-one

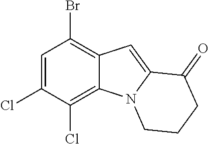 [SMILES: O=C1CCCn2c1cc1c(Br)cc(Cl)c(Cl)c12]

Suspend ethyl 1-bromo-3,4-dichloro-9-oxo-7,8-dihydro-6H-pyrido[1,2-a]indole-8-carboxylate (4.10 g, 9.11 mmol) in acetic acid (30 mL) and add concentrated HCl (10 mL). Stir at 100° C. for 16-18 hrs. Quench with saturated aqueous sodium carbonate to pH=8, extract with DCM, wash with saturated aqueous NaCl, dry over anhydrous sodium sulfate, filter, and concentrate under vacuum to give 1-bromo-3,4-dichloro-7,8-dihydro-6H-pyrido[1,2-a]indol-9-one (2.20 g, 64%) as a yellow solid.

### Intermediate 492

### 1-Bromo-3,4-dichloro-6,7, 8,9-tetrahydropyrido[1,2-a]indol-9-amine

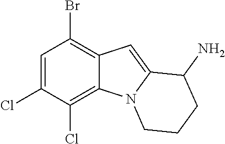 [SMILES: NC1CCCn2c1cc1c(Br)cc(Cl)c(Cl)c12]

Dissolve 1-bromo-3,4-dichloro-7,8-dihydro-6H-pyrido[1,2-a]indol-9-one (1.70 g, 4.59 mmol) and NH₄OAc (5.31 g, 68.9 mmol) in MeOH (10 mL) and THF (10 mL). Add NaBH₃CN (1.52 g, 23.0 mmol) and stir at 60° C. for 16-18 hrs. Quench with saturated aqueous sodium bicarbonate, extract with DCM, wash with saturated aqueous NaCl, dry over anhydrous sodium sulfate, filter, and concentrate under vacuum. Purify by flash column chromatography eluting with MeOH in DCM to give 1-bromo-3,4-dichloro-6,7,8,9-tetrahydropyrido[1,2-a]indol-9-amine (1.20 g, 70%) as a light-yellow oil.

### Intermediate 493

### N-(1-Bromo-3,4-dichloro-6,7,8,9-tetrahydropyrido[1,2-a]indol-9-yl)acetamide

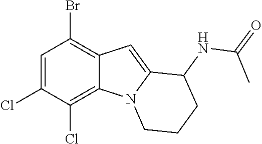 [SMILES: CC(=O)NC1CCCn2c1cc1c(Br)cc(Cl)c(Cl)c12]

Suspend 1-bromo-3,4-dichloro-6,7,8,9-tetrahydropyrido[1,2-a]indol-9-amine (1.20 g, 3.23 mmol) in pyridine (15 mL) and add Ac₂O (0.65 mL, 6.70 mmol). Stir at ambient temperature for 16-18 hrs. Quench with water, extract with EtOAc, wash with saturated aqueous NaCl, dry over anhydrous sodium sulfate, filter, and concentrate under vacuum. Purify by flash column chromatography eluting with MeOH in DCM to give N-(1-bromo-3,4-dichloro-6,7,8,9-tetrahydropyrido[1,2-a]indol-9-yl)acetamide (950 mg, 78%) as a white solid.

### Intermediate 494

### N-(3,4-Dichloro-1-hydroxy-6,7, 8,9-tetrahydropyrido[1,2-a]indol-9-yl)acetamide

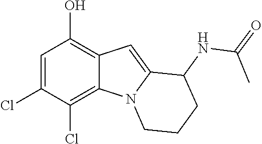 [SMILES: CC(=O)NC1CCCn2c1cc1c(O)cc(Cl)c(Cl)c12]

Suspend N-(1-bromo-3,4-dichloro-6,7,8,9-tetrahydropyrido[1,2-a]indol-9-yl)acetamide (200 mg, 0.532 mmol), t-BuONa (160 mg, 1.63 mmol) and t-BuBrettPhos-Pd-G₃ (48 mg, 0.0551 mmol) in 1,4-dioxane (4 mL) and water (1 mL). Purge with N₂ and heat at 65° C. for 5 hrs. Cool to ambient temperature, quench with 1M aqueous HCl to adjust to pH=4, extract with EtOAc, wash with saturated aqueous NaCl, dry over anhydrous sodium sulfate, filter, and concentrate under vacuum. Purify by flash column chromatography eluting with MeOH in DCM to give N-(3,4-dichloro-1-hydroxy-6,7,8,9-tetrahydropyrido[1,2-a]indol-9-yl)acetamide (90 mg, 49%) as a brown oil. ES/MS (m/z): (³⁵Cl/³⁷Cl) 313.0/315.0 (M+H).

### Intermediate 495

### N-[3,4-Dichloro-1-(cyanomethoxy)-6,7,8,9-tetrahydropyrido[1,2-a]indol-9-yl]acetamide

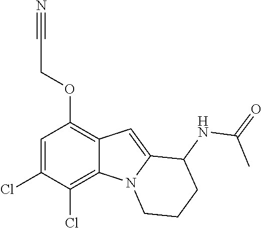 [SMILES: CC(=O)NC1CCCn2c1cc1c(OCC#N)cc(Cl)c(Cl)c12]

Suspend N-(3,4-dichloro-1-hydroxy-6,7,8,9-tetrahydropyrido[1,2-a]indol-9-yl)acetamide (90 mg, 0.26 mmol) and K₂CO₃ (45 mg, 0.33 mmol) in DMF (3 mL). Add bromoacetonitrile (0.040 mL, 0.57 mmol) and stir at 0° C. for 2 hrs. Dilute with EtOAc, wash with water (2×) and saturated aqueous NaCl (2×), dry over anhydrous sodium sulfate, filter, and concentrate under vacuum. Purify by flash column chromatography eluting with MeOH in DCM to give N-[3,4-dichloro-1-(cyanomethoxy)-6,7,8,9-tetrahydropyrido[1,2-a]indol-9-yl]acetamide (80 mg, 87%) as a brown solid. ES/MS (m/z): (³⁵Cl/³⁷Cl) 351.9/353.9 (M+H).

### Intermediate 496

### N-[3,4-Dichloro-1-(cyanomethoxy)-10-iodo-6,7,8,9-tetrahydropyrido[1,2-a]indol-9-yl]acetamide

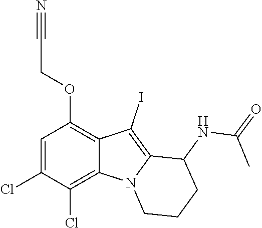 [SMILES: CC(=O)NC1CCCn2c1c(I)c1c(OCC#N)cc(Cl)c(Cl)c12]

Dissolve N-[3,4-dichloro-1-(cyanomethoxy)-6,7,8,9-tetrahydropyrido[1,2-a]indol-9-yl]acetamide (80 mg, 0.23 mmol) in DMF (3 mL) add NIS (83 mg, 0.36 mmol) at 0° C. Stir at ambient temperature for 1 hr. Quench with saturated aqueous sodium thiosulfate, extract with EtOAc, wash with saturated aqueous NaCl, dry over anhydrous sodium sulfate, filter, and concentrate under vacuum. Purify by flash column chromatography eluting with MeOH in DCM to give N-[3,4-dichloro-1-(cyanomethoxy)-10-iodo-6,7,8,9-tetrahydropyrido[1,2-a]indol-9-yl]acetamide (50 mg, 44%) as a light-yellow solid. ES/MS (m/z): (³⁵Cl/³⁷Cl) 478.0/480.0 (M+H).

### Intermediate 497

### N-[3,4-Dichloro-1-(cyanomethoxy)-10-(1-tetrahydropyran-2-ylpyrazol-4-yl)-6, 7,8,9-tetrahydropyrido[1,2-a]indol-9-yl]acetamide

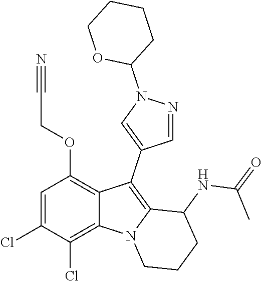 [SMILES: CC(=O)NC1CCCn2c1c(-c1cnn(C3CCCCO3)c1)c1c(OCC#N)cc(Cl)c(Cl)c12]

Suspend N-[3,4-dichloro-1-(cyanomethoxy)-10-iodo-6,7,8,9-tetrahydropyrido[1,2-a]indol-9-yl]acetamide (50 mg, 0.099 mmol), 1-(tetrahydro-2H-pyran-2-yl)-4-(4,4,5,5-tetramethyl-1,3,2-dioxaborolan-2-yl)-1H-pyrazole (45 mg, 0.15 mmol), Na₂CO₃ (32 mg, 0.30 mmol) and Pd(dtbpf)Cl₂ (8 mg, 0.012 mmol) in 1,4-dioxane (4 mL) and water (1 mL). Purge with N₂ and stir at 90° C. for 1 hr. Cool to ambient temperature, dilute with water, extract with EtOAc (2×), wash the combined organic layers with saturated aqueous NaCl, dry over anhydrous sodium sulfate, filter, and concentrate under vacuum. Purify by flash column chromatography eluting with MeOH in DCM to give N-[3,4-dichloro-1-(cyanomethoxy)-10-(1-tetrahydropyran-2-ylpyrazol-4-yl)-6,7,8,9-tetrahydropyrido[1,2-a]indol-9-yl]acetamide (50 mg, 59%) as a brown oil. ES/MS (m/z): (³⁵Cl/³⁷Cl) 502.0/504.0 (M+H).

### EXAMPLE 202

### N-[3,4-Dichloro-1-(cyanomethoxy)-10-(1H-pyrazol-4-yl)-6,7,8,9-tetrahydropyrido[1,2-a]indol-9-yl]acetamide

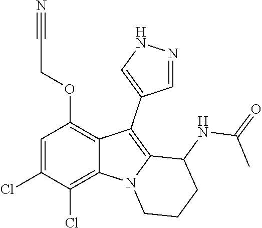 [SMILES: CC(=O)NC1CCCn2c1c(-c1cn[nH]c1)c1c(OCC#N)cc(Cl)c(Cl)c12]

Dissolve N-[3,4-dichloro-1-(cyanomethoxy)-10-(1-tetrahydropyran-2-ylpyrazol-4-yl)-6,7,8,9-tetrahydropyrido[1,2-a]indol-9-yl]acetamide (50 mg, 0.059 mmol) in DCM (2 mL) and TFA (2 mL). Stir at ambient temperature for 1 hr. Dilute with water and adjust to pH=8 with 1M aqueous NaOH. Extract with EtOAc (2×), wash with saturated aqueous NaCl, dry over anhydrous sodium sulfate, filter, and concentrate under vacuum. Purify by prep-HPLC to give N-[3,4-dichloro-1-(cyanomethoxy)-10-(1H-pyrazol-4-yl)-6,7,8,9-tetrahydropyrido[1,2-a]indol-9-yl]acetamide (13.8 mg, 54%) as a white solid. ES/MS (m/z): (³⁵Cl/³⁷Cl) 418.3/420.3 (M+H).

### Intermediate 498

### 9-Bromo-6,7-dichloro-1,2,3,4-tetrahydropyrazino[1,2-a]indole hydrochloride

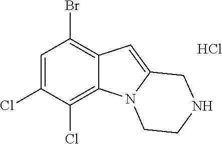 [SMILES: Clc1cc(Br)c2cc3n(c2c1Cl)CCNC3 ; Cl]

Suspend tert-butyl 9-bromo-6,7-dichloro-3,4-dihydropyrazino[1,2-a]indole-2(1H)-carboxylate (500 mg, 1.13 mmol) in 4M HCl in MeOH (10 mL) and stir at ambient temperature for 16-18 hrs. Concentrate under vacuum to give 9-bromo-6,7-dichloro-1,2,3,4-tetrahydropyrazino[1,2-a]indole hydrochloride (365 mg, 90%) as a yellow solid. ES/MS (m/z): 318.8/320.8/322.8 (M+H).

### Intermediate 499

### 9-Bromo-6,7-dichloro-3,4-dihydro-1H-pyrazino e-2-carbothiohydrazide

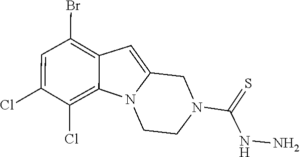 [SMILES: NNC(=S)N1CCn2c(cc3c(Br)cc(Cl)c(Cl)c32)C1]

Dissolve 9-bromo-6,7-dichloro-1,2,3,4-tetrahydropyrazino[1,2-a]indole hydrochloride (658 mg, 1.83 mmol) in THF (5 mL). Add TCDI (573 mg, 3.05 mmol) and stir at ambient temperature for 3 hrs. Then add hydrazine hydrate (123 mg, 3.07 mmol, 80% purity) and stir at 60° C. for 16-18 hrs. Filter the precipitate and wash with water. Dry under vacuum to give 9-bromo-6,7-dichloro-3,4-dihydro-1H-pyrazino[1,2-a]indole-2-carbothiohydrazide (585 mg, 65%) as a brown solid. ES/MS (m/z): 392.9/394.8/396.8 (M+H).

### Intermediate 500

### 2-(9-Bromo-6,7-dichloro-3,4-dihydro-1H-pyrazino[1,2-a]indol-2-yl)-5-methyl-1,3,4-thiadiazole

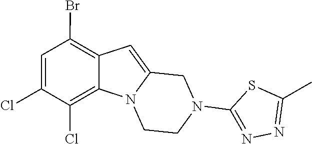 [SMILES: Cc1nnc(N2CCn3c(cc4c(Br)cc(Cl)c(Cl)c43)C2)s1]

Dissolve 9-bromo-6,7-di chloro-3,4-dihydro-1H-pyrazino[1,2-a]indole-2-carbothiohydrazide (585 mg, 1.19 mmol) in acetyl chloride (5 mL) and stir at 0° C. for 16-18 hrs. Quench with 6N aqueous NaOH, filter the precipitate and dry under vacuum to give 2-(9-bromo-6,7-dichloro-3,4-dihydro-1H-pyrazino[1,2-a]indol-2-yl)-5-methyl-1,3,4-thiadiazole (632 mg, 99%) as a yellow solid. ES/MS (m/z): 416.8/418.8/420.8 (M+H).

### Intermediate 501

### 6,7-Dichloro-2-(5-methyl-1,3,4-thiadiazol-2-yl)-3,4-dihydro-1H-pyrazino indol-9-ol

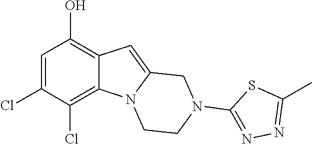 [SMILES: Cc1nnc(N2CCn3c(cc4c(O)cc(Cl)c(Cl)c43)C2)s1]

Suspend 2-(9-bromo-6,7-dichloro-3,4-dihydro-1H-pyrazino[1,2-a]indol-2-yl)-5-methyl-1,3,4-thiadiazole (380 mg, 0.664 mmol), t-BuBrettPhos-Pd-G₃ (29 mg, 0.033 mmol) and sodium tert-butoxide (165 mg, 1.67 mmol) in 1,4-dioxane (4 mL) and water (1 mL). Purge with N₂ and heat at 65° C. for 3 hrs. Cool to ambient temperature, acidify with 1M aqueous HCl to pH=4, extract with EtOAc, wash with saturated aqueous NaCl, dry over sodium sulfate, filter, and concentrate under vacuum. Purify by flash column chromatography eluting with EtOAc in petroleum ether to give 6,7-dichloro-2-(5-methyl-1,3,4-thiadiazol-2-yl)-3,4-dihydro-1H-pyrazino[1,2-a]indol-9-ol (226 mg, 86%) as a yellow solid. ES/MS (m/z): (³⁵Cl/³⁷Cl) 354.9/356.9 (M+H).

### Intermediate 502

### 2-[[6,7-Dichloro-2-(5-methyl-1,3,4-thiadiazol-2-yl)-3,4-dihydro-1H-pyrazino[1,2-a]indol-9-yl]oxy]acetonitrile

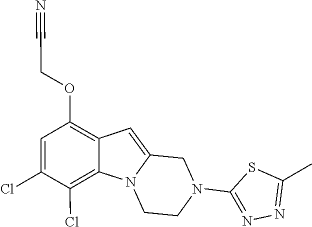 [SMILES: Cc1nnc(N2CCn3c(cc4c(OCC#N)cc(Cl)c(Cl)c43)C2)s1]

Dissolve 6,7-dichloro-2-(5-methyl-1,3,4-thiadiazol-2-yl)-3,4-dihydro-1H-pyrazino[1,2-a]indol-9-ol (226 mg, 0.573 mmol) in DMF (2 mL) and cool to 0° C. Add bromoacetonitrile (142 mg, 1.15 mmol) and potassium carbonate (80 mg, 0.58 mmol) and stir at 0° C. for 2 hrs. Concentrate under vacuum, dilute with EtOAc, and wash with water. Extract the aqueous layer with EtOAc (3×). Wash the combined organic layers with saturated aqueous NaCl, dry over sodium sulfate, filter, and concentrate under vacuum to give 2-[[6,7-di chloro-2-(5-methyl-1,3,4-thiadiazol -2-yl)-3,4-dihydro-1H-pyrazino[1,2-a]indol-9-yl]oxy]acetonitrile (171 mg, 44%) as a brown solid. ES/MS (m/z): (³⁵Cl/³⁷Cl) 393.9/395.9 (M+H).

### Intermediate 503

### 2-[[6,7-Dichloro-10-iodo-2-(5-methyl-1,3,4-thiadiazol-2-yl)-3,4-dihydro-1H-pyrazino[1,2-a]indol-9-yl]oxy]acetonitrile

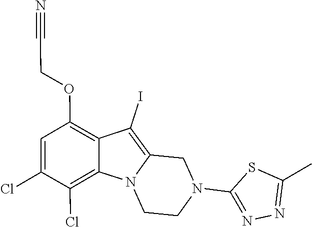 [SMILES: Cc1nnc(N2CCn3c(c(I)c4c(OCC#N)cc(Cl)c(Cl)c43)C2)s1]

Dissolve 2-[[6,7-dichloro-2-(5-methyl-1,3,4-thiadiazol-2-yl)-3,4-dihydro-1H-pyrazino[1,2-a]indol-9-yl]oxy]acetonitrile (171 mg, 0.252 mmol) in DMF (2 mL) and add NIS (90 mg, 0.39 mmol), then stir at ambient temperature for 1.5 hr. Dilute with EtOAc, and wash with water. Extract the aqueous layer with EtOAc (3×). Wash the combined organic layers with saturated aqueous NaCl, dry over sodium sulfate, filter, and concentrate under vacuum to give 2-[[6,7-dichloro-10-iodo-2-(5-methyl-1,3,4-thiadiazol-2-yl)-3,4-dihydro-1H-pyrazino[1,2-a]indol-9-yl]oxy]acetonitrile (206 mg, 99+%) as a brown solid.

### Intermediate 504

### 2-[[6,7-Dichloro-2-(5-methyl-1,3,4-thiadiazol-2-yl)-10-(1-tetrahydropyran-2-ylpyrazol-4-yl)-3,4-dihydro-1H-pyrazino[1,2-a]indol-9-yl]oxy]acetonitrile

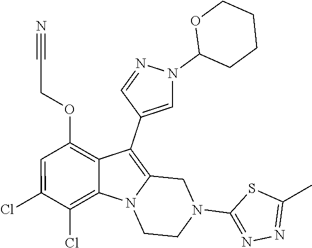 [SMILES: Cc1nnc(N2CCn3c(c(-c4cnn(C5CCCCO5)c4)c4c(OCC#N)cc(Cl)c(Cl)c43)C2)s1]

Suspend 2-[[6,7-dichloro-10-iodo-2-(5-methyl-1,3,4-thiadiazol-2-yl)-3,4-dihydro-1H-pyrazino[1,2-a]indol-9-yl]oxy]acetonitrile (206 mg, 0.317 mmol), 1-(tetrahydro-2H-pyran-2-yl)-4-(4,4,5,5-tetramethyl-1,3,2-dioxaborolan-2-yl)-1H-pyrazole (210 mg, 0.717 mmol), Pd(dtbpf)Cl₂ (50 mg, 0.075 mmol) and sodium carbonate (115 mg, 1.09 mmol) in 1,4-dioxane (4 mL) and water (1 mL). Purge with N₂ and heat at 90° C. for 1 hr. Cool to ambient temperature, dilute with EtOAc, and wash with water. Extract the aqueous layer with EtOAc (3×). Wash the combined organic layers with saturated aqueous NaCl, dry over sodium sulfate, filter, and concentrate under vacuum. Purify by flash column chromatography eluting with EtOAc in petroleum ether to give 2-[[6,7-dichloro-2-(5-methyl-1,3,4-thiadiazol-2-yl)-10-(1-tetrahydropyran-2-ylpyrazol-4-yl)-3,4-dihydro-1H-pyrazino[1,2-a]indol-9-yl]oxy]acetonitrile (183 mg, 67%) as a yellow solid. ES/MS (m/z): 544.1/546.1 (M+H) and 460.0/462.0 (M+H-THP).

### EXAMPLE 203

### 2-((6,7-Dichloro-2-(5-methyl-1,3,4-thiadiazol-2-yl)-10-(1H-pyrazol-4-yl)-1,2,3,4-tetrahydropyrazino[1,2-a]indol-9-yl)oxy)acetonitrile

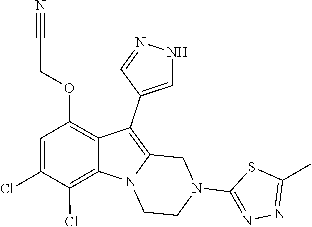 [SMILES: Cc1nnc(N2CCn3c(c(-c4cn[nH]c4)c4c(OCC#N)cc(Cl)c(Cl)c43)C2)s1]

Suspend 2-[[6,7-dichloro-2-(5-methyl-1,3,4-thiadiazol-2-yl)-10-(1-tetrahydropyran-2-ylpyrazol-4-yl)-3,4-dihydro-1H-pyrazino[1,2-a]indol-9-yl]oxy]acetonitrile (183 mg, 0.212 mmol) in DCM (2 mL). Add TFA (3 mL) and stir at ambient temperature for 2 hrs. Concentrate under vacuum and purify by prep-HPLC to give 2-((6,7-di chloro-2-(5-methyl-1,3,4-thiadiazol -2-yl)-10-(1H-pyrazol-4-yl)-1,2,3,4-tetrahydropyrazino[1,2-a]indol-9-yl)oxy)acetonitrile (13.58 mg, 14%) as an off-white solid. ES/MS (m/z): (³⁵Cl/³⁷Cl) 460.2/462.3 (M+H).

### EXAMPLE 204

### 2-[[6,7-Dichloro-10-(1H-pyrazol-4-yl)-2-pyrimidin-2-yl-3,4-dihydro-1H-pyrazino[1,2-a]indol-9-yl]oxy]acetonitrile

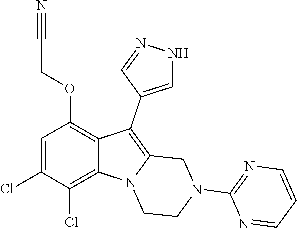 [SMILES: N#CCOc1cc(Cl)c(Cl)c2c1c(-c1cn[nH]c1)c1n2CCN(c2ncccn2)C1]

Dissolve 2-((6,7-dichloro-10-(1H-pyrazol-4-yl)-1,2,3,4-tetrahydropyrazino[1,2-a]indol-9-yl)oxy)acetonitrile (150 mg, 0.365 mmol, HCl salt), 2-chloropyrimidine (130 mg, 1.10 mmol) and DIPEA (0.20 mL, 1.1 mmol) in DMF (2 mL). Stir at 100° C. in a sealed tube for 4 hrs under microwave irradiation. Concentrate under vacuum and purify by prep-HPLC to give 2-[[6,7-dichloro-10-(1H-pyrazol-4-yl)-2-pyrimidin-2-yl-3,4-dihydro-1H-pyrazino[1,2-a]indol-9-yl]oxy]acetonitrile (77.38 mg, 46%) as a pale pink solid. ES/MS (m/z): (³⁵Cl/³⁷Cl) 440.3/442.2 (M+H).

### Intermediate 505

### N-[3-[tert-Butyl(dimethyl)silyl]oxy-2-methyl-propyl]-6,7-dichloro-3-(1H-pyrazol-4-yl)-1H-indol-4-amine

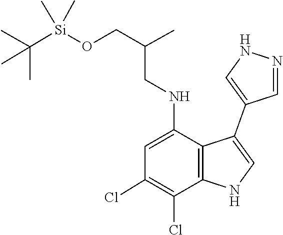 [SMILES: CC(CNc1cc(Cl)c(Cl)c2[nH]cc(-c3cn[nH]c3)c12)CO[Si](C)(C)C(C)(C)C]

Dissolve 6,7-dichloro-3-(1H-pyrazol-4-yl)-1H-indol-4-amine (100 mg, 0.296 mmol, HCl salt) in MeOH (3 mL) and add DIPEA (120 mg, 0.929 mmol) to adjust to pH=9, stir at ambient temperature for 15 min. Add acetic acid (55 mg, 0.91 mmol) to adjust to pH=5 and stir at ambient temperature for 30 min. Add 3-[tert-butyl(dimethyl)silyl]oxy-2-methyl-propanal (CAS: 91751-25-6, 190 mg, 0.892 mmol) and stir at ambient temperature for another 30 min. Add NaBH₃CN (60 mg, 0.91 mmol) and stir at ambient temperature for 16-18 hrs. Combine with another batch run at 0.53×scale for work up and purification. Dilute with water, adjust to pH=7 with saturated aqueous sodium bicarbonate, extract with DCM, wash with saturated aqueous NaCl, dry over anhydrous sodium sulfate, filter, and concentrate under vacuum. Purify by flash column chromatography eluting with EtOAc in petroleum ether to give N-[3-[tert-butyl(dimethyl)silyl]oxy-2-methyl-propyl]-6,7-dichloro-3-(1H-pyrazol-4-yl)-1H-indol-4-amine (150 mg, 59%) as a light-yellow oil. ES/MS (m/z): (³⁵Cl/³⁷Cl) 453.0/455.0 (M+H).

### EXAMPLE 205

### 3-[[6,7-Dichloro-3-(1H-pyrazol-4-yl)-1H-indol-4-yl]amino]-2-methyl-propan-1-ol

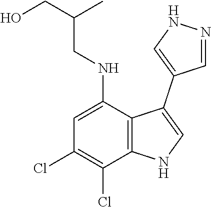 [SMILES: CC(CO)CNc1cc(Cl)c(Cl)c2[nH]cc(-c3cn[nH]c3)c12]

Dissolve N-[3-[tert-butyl(dimethyl)silyl]oxy-2-methyl-propyl]-6,7-dichloro-3-(1H-pyrazol-4-yl)-1H-indol-4-amine (150 mg, 0.268 mmol) in THF (2 mL) and add 6M aqueous HCl (2 mL). Stir at ambient temperature for 1 hr. Concentrate under vacuum and purify by prep-HPLC to give 3-[[6,7-dichloro-3-(1H-pyrazol-4-yl)-1H-indol-4-yl]amino]-2-methyl-propan-1-ol (61.0 mg, 66%) as a white solid. ES/MS (m/z): (³⁵Cl/³⁷Cl) 339.3/341.2 (M+H).

### Intermediate 506

### 6, 7-Dichloro-4-(oxetan-3-yl oxy)-1-(p-tolylsulfonyl)indole

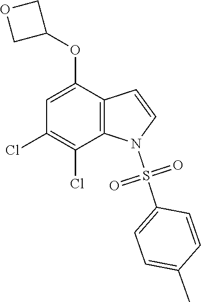

Dissolve 6,7-dichloro-1-(p-tolylsulfonyl)indol-4-ol (500 mg, 1.33 mmol), oxetan-3-ol (210 mg, 2.69 mmol), Ph₃P (893 mg, 3.34 mmol) in toluene (5 mL). Purge with N₂ and add DIAD (0.42 mL, 2.0 mmol). Stir at 110° C. for 16-18 hrs. Dilute with EtOAc, and wash with water. Extract the aqueous layer with EtOAc (3×). Wash the combined organic layers with saturated aqueous NaCl, dry over sodium sulfate, filter, and concentrate under vacuum. Purify by flash column chromatography eluting with EtOAc in petroleum ether to give 6,7-dichloro-4-(oxetan-3-yloxy)-1-(p-tolylsulfonyl)indole (940 mg, 99+%) as a white solid.

### Intermediate 507

### 6,7-Dichloro-4-(oxetan-3-yloxy)-1H-indole

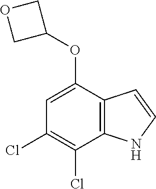 [SMILES: Clc1cc(OC2COC2)c2cc[nH]c2c1Cl]

Dissolve 6,7-dichloro-4-(oxetan-3-yloxy)-1-(p-tolylsulfonyl)indole (940 mg, 1.82 mmol) in THF (5 mL). Add TBAF (6 mL, 1.0M in THF) and stir at ambient temperature for 2 hrs. Dilute with EtOAc, wash with saturated aqueous NH₄Cl, dry over anhydrous sodium sulfate, filter, and concentrate under vacuum. Purify by flash column chromatography eluting with EtOAc in petroleum ether to give 6,7-dichloro-4-(oxetan-3-yloxy)-1H-indole (677 mg, 99+%) as a pale-yellow solid. ES/MS (m/z): 258.0/260.0 (M+H).

### Intermediate 508

### 6,7-Dichloro-3-iodo-4-(oxetan-3-yl oxy)-1H-indole

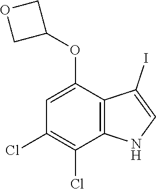 [SMILES: Clc1cc(OC2COC2)c2c(I)c[nH]c2c1Cl]

Dissolve 6,7-dichloro-4-(oxetan-3-yloxy)-1H-indole (677 mg, 2.31 mmol) in DMF (5 mL). Add NIS (636 mg, 2.77 mmol) and stir at ambient temperature for 1.5 hr. Dilute with saturated aqueous Na₂SO₃, filter the precipitate and dry under vacuum to give 6,7-dichloro-3-iodo-4-(oxetan-3-yloxy)-1H-indole (667 mg, 56%) as a pink solid.

### EXAMPLE 206

### 6,7-Dichloro-4-(oxetan-3-yloxy)-3-(1H-pyrazol-4-yl)-1H-indole

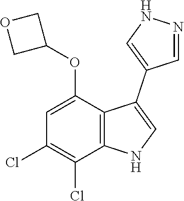 [SMILES: Clc1cc(OC2COC2)c2c(-c3cn[nH]c3)c[nH]c2c1Cl]

Suspend 6,7-dichloro-3-iodo-4-(oxetan-3-yloxy)-1H-indole (600 mg, 1.17 mmol), 1H-pyrazole-4-boronic acid (268 mg, 2.35 mmol), Pd(dtbpf)Cl₂ (157 mg, 0.236 mmol) and sodium carbonate (373 mg, 3.52 mmol) in 1,4-dioxane (4 mL) and water (1 mL). Purge with N₂ and heat at 90° C. for 1 hr. Concentrate under vacuum and purify by prep-HPLC to give 6,7-dichloro-4-(oxetan-3-yloxy)-3-(1H-pyrazol-4-yl)-1H-indole (22.35 mg, 6%) as a white solid. ES/MS (m/z): (³⁵Cl/³⁷Cl) 324.2/326.2 (M+H).

### Intermediate 509

### Cis-tert-Butyl-[3-[6,7-dichloro-1-(p-tolylsulfonyl)indol-4-yl]oxycyclobutoxy]-dimethyl-silane

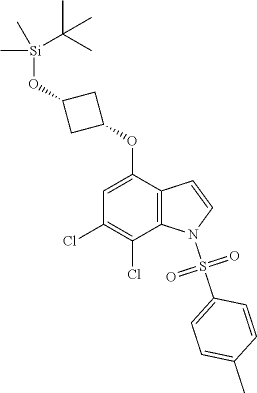 [SMILES: Cc1ccc(S(=O)(=O)n2ccc3c(O[C@H]4C[C@@H](O[Si](C)(C)C(C)(C)C)C4)cc(Cl)c(Cl)c32)cc1]

Dissolve 6,7-dichloro-1-(p-tolylsulfonyl)indol-4-ol (350 mg, 0.934 mmol), trans-3-[[(1,1-dimethylethyl)dimethylsilyl]oxy]cyclobutanol (390 mg, 1.87 mmol), Ph₃P (625 mg, 2.34 mmol) in toluene (3 mL). Purge with N₂. Add DIAD (0.30 mL, 1.4 mmol) and stir at 110° C. for 16-18 hrs. Dilute with EtOAc, and wash with water. Extract the aqueous layer with EtOAc (3×). Wash the combined organic layers with saturated aqueous NaCl, dry over sodium sulfate, filter, and concentrate under vacuum. Purify by flash column chromatography eluting with EtOAc in petroleum ether to give cis-tert-butyl-[3-[6,7-dichloro-1-(p-tolylsulfonyl)indol-4-yl]oxycyclobutoxy]-dimethyl-silane (509 mg, 96%) as a white solid.

### Intermediate 510

### Cis-3-[(6, 7-Dichloro-1H-indol-4-yl)oxy]cyclobutanol

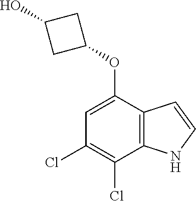 [SMILES: O[C@H]1C[C@@H](Oc2cc(Cl)c(Cl)c3[nH]ccc23)C1]

Dissolve cis-tert-butyl-[3-[6,7-dichloro-1-(p-tolylsulfonyl)indol-4-yl]oxycyclobutoxy]-dimethyl-silane (500 mg, 0.694 mmol) in MeOH (3 mL) and water (3 mL). Add NaOH (282 mg, 6.98 mmol), then stir at 60° C. for 3 days. Dilute with EtOAc, and wash with water. Extract the aqueous layer with EtOAc (3×). Wash the combined organic layers with saturated aqueous NaCl, dry over sodium sulfate, filter, and concentrate under vacuum to give cis-3-[(6,7-dichloro-1H-indol-4-yl)oxy]cyclobutanol (199 mg, 95%) as a yellow solid.

### Intermediate 511

### Cis-3-[(6, 7-Dichloro-3-iodo-1H-indol-4-yl)oxy]cyclobutanol

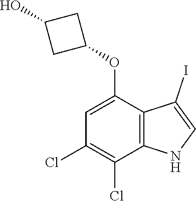 [SMILES: O[C@H]1C[C@@H](Oc2cc(Cl)c(Cl)c3[nH]cc(I)c23)C1]

Dissolve cis-3-[(6,7-dichloro-1H-indol-4-yl)oxy]cyclobutanol (199 mg, 0.658 mmol) in DMF (3 mL). Add NIS (183 mg, 0.797 mmol) and stir at ambient temperature for 1.5 hr. Concentrate under vacuum, dilute with EtOAc, and wash with water. Extract the aqueous layer with EtOAc (3×). Wash the combined organic layers with saturated aqueous NaCl, dry over sodium sulfate, filter, and concentrate under vacuum to give cis-3-[(6,7-dichloro-3-iodo-1H-indol-4-yl)oxy]cyclobutanol (284 mg, 86%) as a brown oil.

### Intermediate 512

### Cis-3-[[6, 7-Dichloro-3-(1-tetrahydropyran-2-ylpyrazol -4-yl)-1H-indol-4-yl]oxy]cyclobutanol

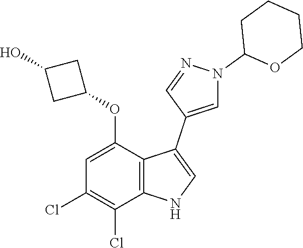 [SMILES: O[C@H]1C[C@@H](Oc2cc(Cl)c(Cl)c3[nH]cc(-c4cnn(C5CCCCO5)c4)c23)C1]

Suspend cis-3-[(6,7-dichloro-3-iodo-1H-indol-4-yl)oxy]cyclobutanol (284 mg, 0.571 mmol), 1-(tetrahydro-2H-pyran-2-yl)-4-(4,4,5,5-tetramethyl-1,3,2-dioxaborolan-2-yl)-1H-pyrazole (335 mg, 1.14 mmol), Pd(dtbpf)Cl₂ (76 mg, 0.11 mmol) and sodium carbonate (182 mg, 1.72 mmol) in 1,4-dioxane (4 mL) and water (1 mL). Purge with N₂ and heat at 90° C. for 2 hrs. Cool to ambient temperature, dilute with EtOAc, and wash with water. Extract the aqueous layer with EtOAc (3×). Wash the combined organic layers with saturated aqueous NaCl, dry over sodium sulfate, filter, and concentrate under vacuum. Purify by flash column chromatography eluting with EtOAc in petroleum ether to give cis-3-[[6,7-dichloro-3-(1-tetrahydropyran-2-ylpyrazol-4-yl)-1H-indol-4-yl]oxy]cyclobutanol (189 mg, 70%) as a yellow solid. ES/MS (m/z): (³⁵Cl/³⁷Cl) 422.1/424.0 (M+H).

### EXAMPLE 207

### Cis-3-[[6,7-Dichloro-3-(1H-pyrazol-4-yl)-1H-indol-4-yl]oxy]cyclobutanol

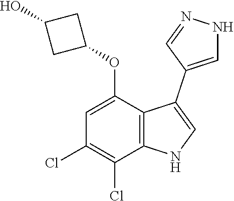 [SMILES: O[C@H]1C[C@@H](Oc2cc(Cl)c(Cl)c3[nH]cc(-c4cn[nH]c4)c23)C1]

Suspend cis-3-[[6,7-dichloro-3-(1-tetrahydropyran-2-ylpyrazol-4-yl)-1H-indol-4-yl]oxy]cyclobutanol (189 mg, 0.398 mmol) in THF (2 mL). Add 6M aqueous HCl (4 mL) and stir at ambient temperature for 2 hrs. Concentrate under vacuum and purify by prep-HPLC to give cis-3-[[6,7-dichloro-3-(1H-pyrazol-4-yl)-1H-indol-4-yl]oxy]cyclobutanol (40.21 mg, 30%) as a white solid. ES/MS (m/z): (³⁵Cl/³⁷Cl) 338.3/340.2 (M+H).

### Intermediate 513

### Cis-6,7-Dichloro-N-(3-fluorocyclobutyl)-3-(1-tetrahydropyran-2-ylpyrazol-4-yl)-1H-indol-4-amine

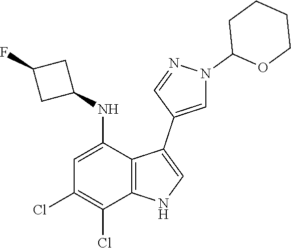 [SMILES: F[C@H]1C[C@@H](Nc2cc(Cl)c(Cl)c3[nH]cc(-c4cnn(C5CCCCO5)c4)c23)C1]

Suspend 4-bromo-6,7-dichloro-1-(p-tolylsulfonyl)-3-(1-tetrahydropyran-2-ylpyrazol-4-yl)indole (200 mg, 0.334 mmol), cis-3-fluorocyclobutanamine hydrochloride (70 mg, 0.54 mmol), t-BuONa (100 mg, 1.01 mmol), (t-Bu₃P)₂Pd (30 mg, 0.058 mmol) in 1,4-dioxane (5 mL). Purge with N₂ and heat to 100° C. for 16-18 hrs. Combine with another batch run at 0.50×scale for work up and purification. Cool to ambient temperature and concentrate under vacuum. Purify by flash column chromatography eluting with EtOAc in petroleum ether to give cis-6,7-dichloro-N-(3-fluorocyclobutyl)-3-(1-tetrahydropyran-2-ylpyrazol-4-yl)-1H-indol-4-amine (120 mg, 44%) as a yellow gum. ES/MS (m/z): (³⁵Cl/³⁷Cl) 423.0/425.0 (M+H).

### EXAMPLE 208

### Cis-6,7-Dichloro-N-(3-fluorocyclobutyl)-3-(1H-pyrazol-4-yl)-1H-indol-4-amine

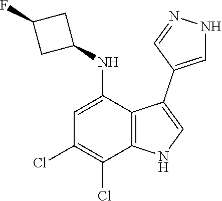 [SMILES: F[C@H]1C[C@@H](Nc2cc(Cl)c(Cl)c3[nH]cc(-c4cn[nH]c4)c23)C1]

Dissolve cis-6,7-dichloro-N-(3-fluorocyclobutyl)-3-(1-tetrahydropyran-2-ylpyrazol-4-yl)-1H-indol-4-amine (120 mg, 0.218 mmol) in DCM (2 mL) and add TFA (2 mL). Stir at ambient temperature for 2 hrs. Concentrate under vacuum and purify by prep-HPLC to give cis-6,7-Dichloro-N-(3-fluorocyclobutyl)-3-(1H-pyrazol-4-yl)-1H-indol-4-amine (36.66 mg, 50%) as a white solid. ES/MS (m/z): (³⁵Cl/³⁷Cl) 339.3/341.3 (M+H).

### Intermediate 514

### N-[3-[tert-Butyl(dimethyl)silyl]oxycyclopentyl]-6, 7-dichloro-3-(1-tetrahydropyran-2-ylpyrazol -4-yl)-1H-indol-4-amine

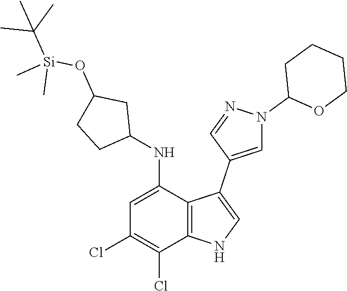 [SMILES: CC(C)(C)[Si](C)(C)OC1CCC(Nc2cc(Cl)c(Cl)c3[nH]cc(-c4cnn(C5CCCCO5)c4)c23)C1]

Suspend 4-bromo-6,7-dichloro-1-(p-tolylsulfonyl)-3-(1-tetrahydropyran-2-ylpyrazol-4-yl)indole (3.5 g, 5.8 mmol), cis-3-[tert-butyl(dimethyl)silyl]oxycyclopentanamine (2.3 g, 9.6 mmol), t-BuONa (1.75 g, 17.7 mmol) and (t-Bu₃P)₂Pd (500 mg, 0.959 mmol) in 1,4-dioxane (50 mL). Purge with N₂ and heat to 100° C. for 16-18 hrs. Dilute with water, extract with EtOAc, wash with saturated aqueous NaCl, dry over anhydrous sodium sulfate, filter, and concentrate under vacuum. Purify by flash column chromatography eluting with EtOAc in petroleum ether to give N-[3-[tert-butyl(dimethyl)silyl]oxycyclopentyl]-6,7-dichloro-3-(1-tetrahydropyran-2-ylpyrazol-4-yl)-1H-indol-4-amine (1.0 g, 24%) as a yellow gum. Note: partial epimerization occurs under reaction conditions to give a mixture of cis and trans isomers. ES/MS (m/z): (³⁵Cl/³⁷Cl) 549.3/551.1 (M+H).

### EXAMPLE 209

### trans-3-[[6,7-Dichloro-3-(1H-pyrazol-4-yl)-1H-indol-4-yl]amino]cyclopentanol (racemic mixture)

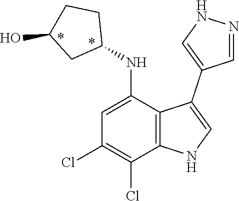 [SMILES: O[C@H]1CC[C@H](Nc2cc(Cl)c(Cl)c3[nH]cc(-c4cn[nH]c4)c23)C1]

Dissolve N-[3-[tert-Butyl(dimethyl)silyl]oxycyclopentyl]-6,7-dichloro-3-(1-tetrahydropyran-2-ylpyrazol-4-yl)-1H-indol-4-amine (1.00 g, 1.40 mmol) in THF (10 mL) and add 6M aqueous HCl (20 mL). Stir at ambient temperature for 2 hrs. Neutralize with saturated aqueous sodium bicarbonate, extract with EtOAc, dry over anhydrous sodium sulfate, filter, and concentrate under vacuum.

Purify by prep-HPLC to give trans-3-[[6,7-dichloro-3-(1H-pyrazol-4-yl)-1H-indol-4-yl]amino]cyclopentanol (15.05 mg, 3%) as a yellow solid (racemic mixture, retention time=1.83 min). ES/MS (m/z): 351.3/353.2 (M+H). Elute further to give cis-3-[[6,7-dichloro-3-(1H-pyrazol-4-yl)-1H-indol-4-yl]amino]cyclopentanol (racemic mixture, retention time=1.98 min). Column: Welch Xtimate C18 150×30 mm; Particle Size: 5 μm; Mobile Phase: A: formic acid in water, B: ACN; Gradient: 0 to 60% B in 14 minutes; Flow Rate: 30 ml/min.

### EXAMPLE 210

### Cis-3-[[6,7-Dichloro-3-(1H-pyrazol-4-yl)-1H-indol-4-yl]amino]cyclopentanol, isomer 2

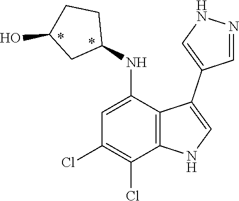 [SMILES: O[C@H]1CC[C@@H](Nc2cc(Cl)c(Cl)c3[nH]cc(-c4cn[nH]c4)c23)C1]

### Isomer 2

Separate the enantiomers of cis-3-[[6,7-dichloro-3-(1H-pyrazol-4-yl)-1H-indol-4-yl]amino]cyclopentanol (from Example 209) by SFC to give cis-3-[[6,7-dichloro-3-(1H-pyrazol-4-yl)-1H-indol-4-yl]amino]cyclopentanol, isomer 2 (167.97 mg, 34%, second eluting compound, retention time=2.77 min) as a white solid. ES/MS (m/z): (³⁵Cl/³⁷Cl) 351.3/353.2 (M+H). Column: Chiralpak AD, 50×150 mm; Particle Size: 3 μm; Mobile Phase: A: CO₂, B: ethanol (0.05% DEA); Gradient: 5% to 40% B in 2.5 min, hold at 40% B for 0.5 min, then 5% of B for 1 min; Flow Rate: 4 mL/min; Column temperature: 35° C.

### Intermediate 515

### tert-Butyl-[2-[5-[6,7-dichloro-3-(1-tetrahydropyran-2-ylpyrazol-4-yl)indol-1-yl]isoxazol -3-yl]ethoxy]-dimethyl-silane

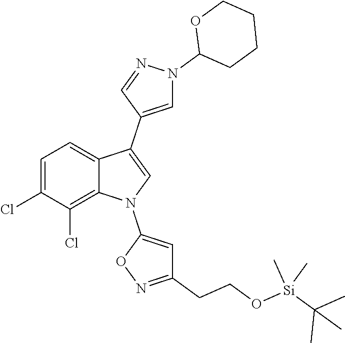 [SMILES: CC(C)(C)[Si](C)(C)OCCc1cc(-n2cc(-c3cnn(C4CCCCO4)c3)c3ccc(Cl)c(Cl)c32)on1]

Dissolve 6,7-dichloro-1-ethynyl-3-(1-tetrahydropyran-2-ylpyrazol-4-yl)indole (300 mg, 0.750 mmol) in THF (6 mL). Add CuI (30 mg, 0.16 mmol), potassium carbonate (210 mg, 1.52 mmol). Purge with N₂. Add (1Z)-3-[tert-butyl(dimethyl)silyl]oxy-N-hydroxy-propanimidoyl chloride (300 mg, 1.26 mmol) under N₂ atmosphere at 0° C. Stir at 75° C. for 6 hrs. Dilute with water, extract with DCM (3×), wash the combined organic layers with saturated aqueous NaCl, dry over anhydrous Na₂SO₄, filter, and concentrate under vacuum. Purify by flash column chromatography eluting with EtOAc in petroleum ether to give tert-butyl-[2-[5-[6,7-dichloro-3-(1-tetrahydropyran-2-ylpyrazol-4-yl)indol-1-yl]isoxazol-3-yl]ethoxy]-dimethyl-silane (290 mg, 68%) as a colorless oil. ES/MS (m/z): (³⁵Cl/³⁷Cl) 561.0/563.0 (M+H) and 476.9/478.9 (M+H-THP).

### EXAMPLE 211

### 2-[5-[6,7-Dichloro-3-(1H-pyrazol-4-yl)indol-1-yl]isoxazol-3-yl]ethanol

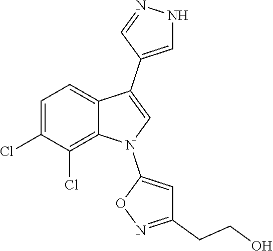 [SMILES: OCCc1cc(-n2cc(-c3cn[nH]c3)c3ccc(Cl)c(Cl)c32)on1]

Suspend tert-butyl-[2-[5-[6,7-dichloro-3-(1-tetrahydropyran-2-ylpyrazol-4-yl)indol-1-yl]isoxazol-3-yl]ethoxy]-dimethyl-silane (289 mg, 0.494 mmol) in THF (2 mL) and add 6M aqueous HCl (1 mL). Stir at ambient temperature for 2 hrs. Concentrate under vacuum and purify by prep-HPLC to give 2-[5-[6,7-dichloro-3-(1H-pyrazol-4-yl)indol-1-yl]isoxazol-3-yl]ethanol (56.0 mg, 31%) as a white solid. ES/MS (m/z): (³⁵Cl/³⁷Cl) 363.2/365.2 (M+H).

### Intermediate 516

### Ethyl 2-[4-[6,7-dichloro-3-(1-tetrahydropyran-2-ylpyrazol-4-yl)indol-1-yl]triazol-1-yl]acetate

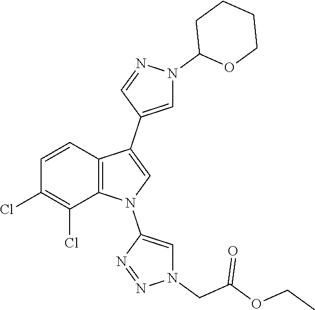 [SMILES: CCOC(=O)Cn1cc(-n2cc(-c3cnn(C4CCCCO4)c3)c3ccc(Cl)c(Cl)c32)nn1]

Dissolve 6,7-dichloro-1-ethynyl-3-(1-tetrahydropyran-2-ylpyrazol-4-yl)indole (380 mg, 1.03 mmol) in DMF (5 mL) and water (5 mL). Add copper (15 mg, 0.24 mmol), cupric sulfate (35 mg, 0.22 mmol) and ethyl azidoacetate (210 mg, 1.59 mmol). Purge with N₂ and stir at 110° C. for 0.5 hr. Dilute with water, extract with EtOAc (3×), dry the combined organic layers over anhydrous Na₂SO₄, filter, and concentrate under vacuum. Purify by flash column chromatography eluting with EtOAc in petroleum ether to give ethyl 2-[4-[6,7-dichloro-3-(1-tetrahydropyran-2-ylpyrazol-4-yl)indol-1-yl]triazol-1-yl]acetate (256 mg, 49%) as a yellow solid. ES/MS (m/z): (³⁵Cl/³⁷Cl) 489.0/491.0 (M+H).

### Intermediate 517

### Ethyl 2-[4-[6,7-dichloro-3-(1H-pyrazol-4-yl)indol-1-yl]triazol-1-yl]acetate

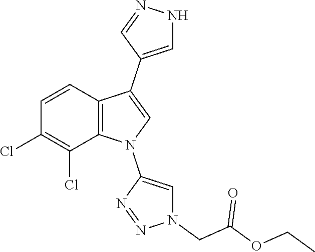 [SMILES: CCOC(=O)Cn1cc(-n2cc(-c3cn[nH]c3)c3ccc(Cl)c(Cl)c32)nn1]

Dissolve ethyl 2-[4-[6,7-dichloro-3-(1-tetrahydropyran-2-ylpyrazol-4-yl)indol-1-yl]triazol-1-yl]acetate (220 mg, 0.418 mmol) in DCM (12 mL) and add TFA (6 mL). Stir at ambient temperature for 3 hrs., and concentrate under vacuum to give ethyl 2-[4-[6,7-dichloro-3-(1H-pyrazol-4-yl)indol-1-yl]triazol-1-yl]acetate (200 mg, 94%) as a brown solid. ES/MS (m/z): (³⁵Cl/³⁷Cl) 404.9/406.8 (M+H).

### EXAMPLE 212

### 2-[4-[6,7-Dichloro-3-(1H-pyrazol-4-yl)indol-1-yl]triazol-1-yl]acetic acid

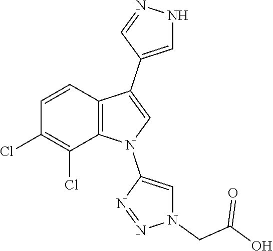 [SMILES: O=C(O)Cn1cc(-n2cc(-c3cn[nH]c3)c3ccc(Cl)c(Cl)c32)nn1]

Suspend ethyl 2-[4-[6,7-dichloro-3-(1H-pyrazol-4-yl)indol-1-yl]triazol-1-yl]acetate (350 mg, 0.864 mmol) in water (5 mL) and THF (5 mL) and add LiOH (400 mg, 9.06 mmol). Stir at ambient temperature for 5 hrs. Dilute with water and acidify with 1M aqueous HCl to pH=4, extract with EtOAc (3×), dry over anhydrous sodium sulfate, filter, and concentrate under vacuum. Purify by prep-HPLC to give 2-[4-[6,7-dichloro-3-(1H-pyrazol-4-yl)indol-1-yl]triazol-1-yl]acetic acid (34.0 mg, 10%) as a white solid. ES/MS (m/z): 377.3/379.2 (M+H).

### EXAMPLE 213

### 2-[4-[6,7-Dichloro-3-(1H-pyrazol-4-yl)indol-1-yl]triazol-1-yl]acetamide

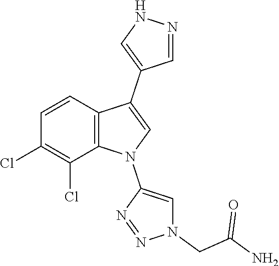 [SMILES: NC(=O)Cn1cc(-n2cc(-c3cn[nH]c3)c3ccc(Cl)c(Cl)c32)nn1]

Suspend 2-[4-[6,7-dichloro-3-(1H-pyrazol-4-yl)indol-1-yl]triazol-1-yl]acetic acid (210 mg, 0.529 mmol), ammonium chloride (35 mg, 0.65 mmol) and DIPEA (210 mg, 1.62 mmol) in DMF (5 mL). Add HATU (250 mg, 0.644 mmol) and stir at ambient temperature for 2 hrs. Concentrate under vacuum and purify by prep-HPLC to give 2-[4-[6,7-dichloro-3-(1H-pyrazol-4-yl)indol-1-yl]triazol-1-yl]acetamide (25.2 mg, 12%) as a white solid. ES/MS (m/z): (³⁵Cl/³⁷Cl) 376.3/378.3 (M+H).

### Intermediate 518

### 2-[2-[4-[6,7-Dichloro-3-(1-tetrahydropyran-2-ylpyrazol-4-yl)indol-1-yl]triazol-1-yl]ethyl]isoindoline-1,3-dione

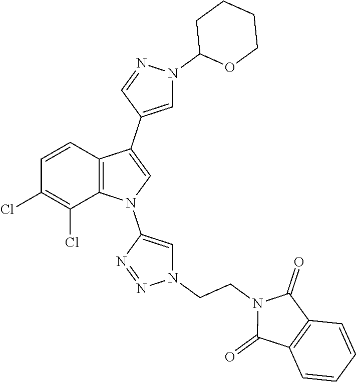 [SMILES: O=C1c2ccccc2C(=O)N1CCn1cc(-n2cc(-c3cnn(C4CCCCO4)c3)c3ccc(Cl)c(Cl)c32)nn1]

Dissolve 6,7-dichloro-1-ethynyl-3-(1-tetrahydropyran-2-ylpyrazol-4-yl)indole (200 mg, 0.528 mmol) in DMF (3 mL) and water (3 mL). Add copper (8 mg, 0.13 mmol), cupric sulfate (18 mg, 0.11 mmol) and 2-(2-azidoethyl)isoindoline-1,3-dione (830 mg, 0.960 mmol, 25% purity). Purge with N₂ and stir at 110° C. for 0.5 hr. Dilute with water, extract with EtOAc, wash with saturated aqueous NaCl, dry over anhydrous sodium sulfate, filter, and concentrate under vacuum. Purify by flash column chromatography eluting with EtOAc in petroleum ether to give 2-[2-[4-[6,7-dichloro-3-(1-tetrahydropyran-2-ylpyrazol-4-yl)indol-1-yl]triazol-1-yl]ethyl]isoindoline-1,3-dione (310 mg, 98%) as a yellow oil. ES/MS (m/z): (³⁵Cl/³⁷Cl) 576.3/578.2 (M+H).

### Intermediate 519

### 2-[4-[6,7-Dichloro-3-(1-tetrahydropyran-2-ylpyrazol-4-yl)indol-1-yl]triazol-1-yl]ethanamine

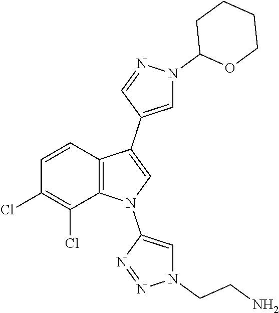 [SMILES: NCCn1cc(-n2cc(-c3cnn(C4CCCCO4)c3)c3ccc(Cl)c(Cl)c32)nn1]

Dissolve 2-[2-[4-[6,7-dichloro-3-(1-tetrahydropyran-2-ylpyrazol-4-yl)indol-1-yl]triazol-1-yl]ethyl]isoindoline-1,3-dione (300 mg, 0.500 mmol) in EtOH (6 mL) and add hydrazine hydrate (250 mg, 6.24 mmol, 80% purity). Stir at 65° C. for 16 hrs. Filter and concentrate the filtrate under vacuum to give 2-[4-[6,7-dichloro-3-(1-tetrahydropyran-2-ylpyrazol-4-yl)indol-1-yl]triazol-1-yl]ethanamine (280 mg, 88%) as a yellow solid. ES/MS (m/z): (³⁵Cl/³⁷Cl) 446.0/447.9 (M+H).

### EXAMPLE 214

### 2-[4-[6,7-Dichloro-3-(1H-pyrazol-4-yl)indol-1-yl]triazol-1-yl]ethanamine hydrochloride

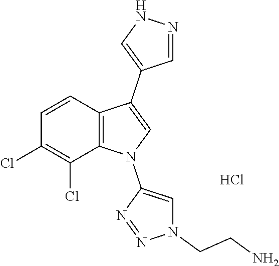 [SMILES: NCCn1cc(-n2cc(-c3cn[nH]c3)c3ccc(Cl)c(Cl)c32)nn1 ; Cl]

Suspend 2-[4-[6,7-dichloro-3-(1-tetrahydropyran-2-ylpyrazol-4-yl)indol-1-yl]triazol-1-yl]ethanamine (280 mg, 0.439 mmol) in 4M HCl in MeOH (5 mL). Stir at ambient temperature for 2 hrs. and concentrate under vacuum. Purify by prep-HPLC to give 2-[4-[6,7-dichloro-3-(1H-pyrazol-4-yl)indol-1-yl]triazol-1-yl]ethanamine hydrochloride (142 mg, 81%) as a white solid. ES/MS (m/z): (³⁵Cl/³⁷Cl) 362.3/364.3 (M+H).

### Intermediate 520

### tert-Butyl N-[[6,7-dichloro-3-(1-tetrahydropyran-2-ylpyrazol-4-yl)-1H-indol-4-yl]amino]-N-methyl-carbamate

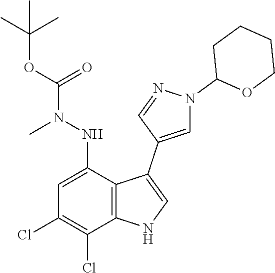 [SMILES: CN(Nc1cc(Cl)c(Cl)c2[nH]cc(-c3cnn(C4CCCCO4)c3)c12)C(=O)OC(C)(C)C]

Suspend 4-bromo-6,7-dichloro-1-(p-tolylsulfonyl)-3-(1-tetrahydropyran-2-ylpyrazol-4-yl)indole (500 mg, 0.834 mmol), 1-Boc-1-methylhydrazine (200 mg, 1.33 mmol), t-BuONa (250 mg, 2.52 mmol), (t-Bu₃P)₂Pd (70 mg, 0.13 mmol) in 1,4-dioxane (10 mL). Purge with N₂ and heat to 100° C. for 16-18 hrs. Cool to ambient temperature and concentrate under vacuum. Purify by flash silica gel chromatography eluting with EtOAc in petroleum ether to give tert-butyl N-[[6,7-dichloro-3-(1-tetrahydropyran-2-ylpyrazol-4-yl)-1H-indol-4-yl]amino]-N-methyl-carbamate (100 mg, 24%) as a yellow solid. ES/MS (m/z): (³⁵Cl/³⁷Cl) 480.4 (M+H, small peak), 424.3/426.3 (M+H-tBu), and 340.2/342.2 (M+H-tBu-THP).

### EXAMPLE 215

### 1-[6,7-Dichloro-3-(1H-pyrazol-4-yl)-1H-indol-4-yl]-2-methyl-hydrazine hydrochloride

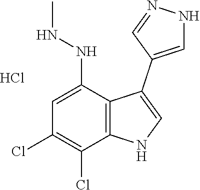 [SMILES: Cl ; CNNc1cc(Cl)c(Cl)c2[nH]cc(-c3cn[nH]c3)c12]

Dissolve tert-butyl N-[[6,7-dichloro-3-(1-tetrahydropyran-2-ylpyrazol-4-yl)-1H-indol-4-yl]amino]-N-methyl-carbamate (100 mg, 0.198 mmol) in 4M HCl in MeOH (3 mL). Stir at ambient temperature for 2 hrs. Filter and wash the precipitate with MeOH to give 1-[6,7-dichloro-3-(1H-pyrazol-4-yl)-1H-indol-4-yl]-2-methyl-hydrazine hydrochloride (60.02 mg, 82%) as a yellow solid. ES/MS (m/z): (³⁵Cl/³⁷Cl) 296.3/298.1 (M+H).

### Intermediate 521

### tert-Butyl (2-(((6, 7-dichloro-3-(1-(tetrahydro-2H-pyran-2-yl)-1H-pyrazol-4-yl)-1H-indol-2-yl)methyl)amino)-2-oxoethyl)carbamate

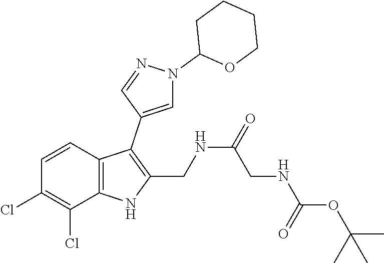 [SMILES: CC(C)(C)OC(=O)NCC(=O)NCc1[nH]c2c(Cl)c(Cl)ccc2c1-c1cnn(C2CCCCO2)c1]

Dissolve [6,7-dichloro-3-(1-tetrahydropyran-2-ylpyrazol-4-yl)-1H-indol-2-yl]methanamine (150 mg, 0.367 mmol), Boc-Gly-OH (50 mg, 0.29 mmol), DIPEA (0.22 mL, 1.2 mmol) and HATU (320 mg, 0.824 mmol) in DMF (6 mL), then stir at ambient temperature for 2 hrs. Concentrate under vacuum, dilute with water, extract with EtOAc (3×), filter the combined organic layers, and concentrate under vacuum. Purify by flash column chromatography eluting with EtOAc in petroleum ether to give tert-butyl (2-(((6,7-dichloro-3-(1-(tetrahydro-2H-pyran-2-yl)-1H-pyrazol-4-yl)-1H-indol-2-yl)methyl)amino)-2-oxoethyl)carbamate (220 mg, 95%) as a light-yellow oil.

### EXAMPLE 216

### 2-Amino-N-[[6,7-dichloro-3-(1H-pyrazol-4-yl)-1H-indol-2-yl]methyl]acetamide formic acid salt

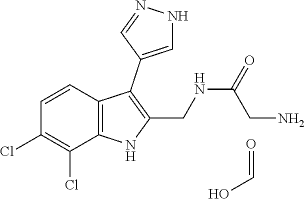

Dissolve tert-butyl (2-(((6,7-dichloro-3-(1-(tetrahydro-2H-pyran-2-yl)-1H-pyrazol-4-yl)-1H-indol-2-yl)methyl)amino)-2-oxoethyl)carbamate (220 mg, 0.295 mmol) in 4M HCl in MeOH (3 mL) and stir at 25° C. for 2 hrs. Concentrate under vacuum and purify by prep-HPLC to give 2-amino-N-[[6,7-dichloro-3-(1H-pyrazol-4-yl)-1H-indol-2-yl]methyl]acetamide formic acid salt (61.5 mg, 62%) as a white solid. ES/MS (m/z): (³⁵Cl/³⁷Cl) 338.3/340.3 (M+H).

### Intermediate 522

### tert-Butyl N-[2-[[6,7-dichloro-3-(1H-pyrazol-4-yl)-1H-indol-2-yl]methylamino]-2-oxo-ethyl]-N-methyl-carbamate

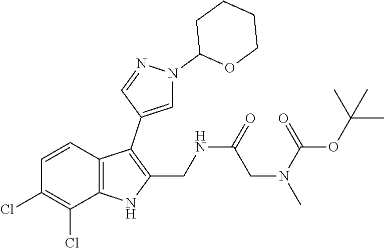 [SMILES: CN(CC(=O)NCc1[nH]c2c(Cl)c(Cl)ccc2c1-c1cnn(C2CCCCO2)c1)C(=O)OC(C)(C)C]

Dissolve [6,7-dichloro-3-(1-tetrahydropyran-2-ylpyrazol-4-yl)-1H-indol-2-yl]methanamine (150 mg, 0.369 mmol), Boc-Sar-OH (150 mg, 0.785 mmol), DIPEA (0.22 mL, 1.2 mmol) and HATU (320 mg, 0.825 mmol) in DMF (6 mL), then stir at ambient temperature for 2 hrs. Concentrate under vacuum, dilute with water, extract with EtOAc (3×), filter the combined organic layers, and concentrate under vacuum. Purify by flash column chromatography eluting with EtOAc in petroleum ether to give tert-butyl N-[2-[[6,7-dichloro-3-(1H-pyrazol-4-yl)-1H-indol-2-yl]methylamino]-2-oxo-ethyl]-N-methyl-carbamate (210 mg, 88%) as a light-yellow oil.

### EXAMPLE 217

### N-[[6, 7-Dichloro-3-(1H-pyrazol-4-yl)-1H-indol-2-yl]methyl]-2-(methyl amino)acetamide formic acid salt

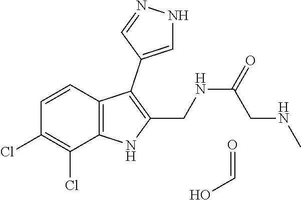

Suspend tert-butyl N-[2-[[6,7-dichloro-3-(1H-pyrazol-4-yl)-1H-indol-2-yl]methylamino]-2-oxo-ethyl]-N-methyl-carbamate (260 mg, 0.339 mmol) in 4M HCl in EtOAc (3 mL) and stir at ambient temperature for 2 hrs. Concentrate under vacuum and purify by prep-HPLC to give N-[[6,7-dichloro-3-(1H-pyrazol-4-yl)-1H-indol-2-yl]methyl]-2-(methylamino)acetamide formic acid salt (58.62 mg, 49%) as a white solid. ES/MS (m/z): (³⁵Cl/³⁷Cl) 352.3/354.3 (M+H).

### Intermediate 523

### 6-Chloro-7-fluoro-3-iodo-1H-indole

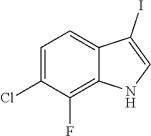 [SMILES: Fc1c(Cl)ccc2c(I)c[nH]c12]

Dissolve 6-chloro-7-fluoro-1H-indole (CAS No. 259860-04-3) (1.00 g, 5.90 mmol) in DMF (10 mL) and add NIS (1.37 g, 5.97 mmol). Stir at ambient temperature for 2 hrs. Dilute with EtOAc, wash with saturated aqueous Na₂SO₃ (2×) and saturated aqueous NaCl, dry over anhydrous sodium sulfate, filter, and concentrate under vacuum. Purify by flash column chromatography eluting with EtOAc in petroleum ether to give 6-chloro-7-fluoro-3-iodo-1H-indole (1.85 g, 96%) as a black solid. ES/MS (m/z): 296.1 (M+H).

### Intermediate 524

### 6-Chloro-7-fluoro-3-(1-tetrahydropyran-2-ylpyrazol-4-yl)-1H-indole

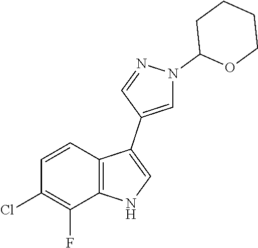 [SMILES: Fc1c(Cl)ccc2c(-c3cnn(C4CCCCO4)c3)c[nH]c12]

Suspend 6-chloro-7-fluoro-3-iodo-1H-indole (1.85 g, 5.63 mmol), 1-(tetrahydro-2H-pyran-2-yl)-4-(4,4,5,5-tetramethyl-1,3,2-dioxaborolan-2-yl)-1H-pyrazole (2.5 g, 8.8 mmol), Pd(dtbpf)Cl₂ (380 mg, 0.577 mmol) and sodium carbonate (1.8 g, 17 mmol) in 1,4-dioxane (24 mL) and water (6 mL). Purge with N₂ and stir at 90° C. for 2 hrs. Dilute with water, extract with EtOAc (3×), wash the combined organic layers with saturated aqueous NaCl, dry over anhydrous sodium sulfate, filter, and concentrate under vacuum. Purify by flash column chromatography eluting with EtOAc in petroleum ether to give 6-chloro-7-fluoro-3-(1-tetrahydropyran-2-ylpyrazol-4-yl)-1H-indole (1.75 g, 95%) as a brown solid. ES/MS (m/z): (³⁵Cl/³⁷Cl) 319.9/321.9 (M+H) and 235.9/237.8 (M+H-THP).

### Intermediate 525

### 2-[6-Chloro-7-fluoro-3-(1-tetrahydropyran-2-ylpyrazol-4-yl)indol-1-yl]ethynyl-triisopropyl-silane

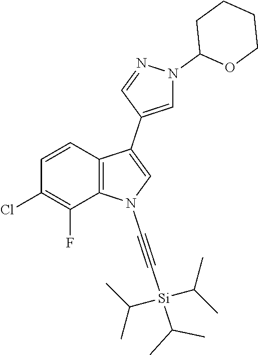 [SMILES: CC(C)[Si](C#Cn1cc(-c2cnn(C3CCCCO3)c2)c2ccc(Cl)c(F)c21)(C(C)C)C(C)C]

Add 6-chloro-7-fluoro-3-(1-tetrahydropyran-2-ylpyrazol-4-yl)-1H-indole (1.20 g, 3.38 mmol) in 1,4-dioxane (20 mL). Add (bromoethynyl)triisopropylsilane (4.60 g, 17 mmol), PEG-400 (500 mg), CuI (70 mg, 0.37 mmol) and Cs₂CO₃ (1.35 g, 4.14 mmol). Purge with N₂ and stir at 130° C. for 16 hrs. Dilute with water, extract with EtOAc (3×), dry the combined organic layers over anhydrous sodium sulfate, filter, and concentrate under vacuum. Purify by flash column chromatography eluting with EtOAc in petroleum ether to give 2-[6-chloro-7-fluoro-3-(1-tetrahydropyran-2-ylpyrazol-4-yl)indol-1-yl]ethynyl-triisopropyl-silane (770 mg, 45%) as a yellow oil.

### Intermediate 526

### 6-Chloro-1-ethynyl-7-fluoro-3-(1-tetrahydropyran-2-ylpyrazol-4-yl)indole

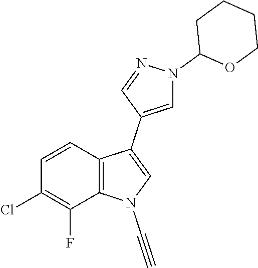 [SMILES: C#Cn1cc(-c2cnn(C3CCCCO3)c2)c2ccc(Cl)c(F)c21]

Dissolve 2-[6-chloro-7-fluoro-3-(1-tetrahydropyran-2-ylpyrazol-4-yl)indol-1-yl]ethynyl-triisopropyl-silane (770 mg, 1.51 mmol) in THF (10 mL) and add TBAF (10 mL, 1.0M in THF). Stir at ambient temperature for 2 hrs. Dilute with EtOAc, wash with saturated aqueous NH₄Cl, dry over anhydrous sodium sulfate, filter, and concentrate under vacuum. Purify by flash column chromatography eluting with EtOAc in petroleum ether to give 6-chloro-1-ethynyl-7-fluoro-3-(1-tetrahydropyran-2-ylpyrazol-4-yl)indole (490 mg, 93%) as a yellow solid. ES/MS (m/z): (³⁵Cl/³⁷Cl) 344.0/345.8 (M+H).

### Intermediate 527

### tert-Butyl-[2-[4-[6-chloro-7-fluoro-3-(1-tetrahydropyran-2-ylpyrazol-4-yl)indol-1-yl]triazol-1-yl]ethoxy]-dimethyl-silane

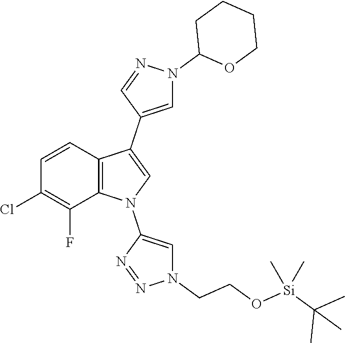 [SMILES: CC(C)(C)[Si](C)(C)OCCn1cc(-n2cc(-c3cnn(C4CCCCO4)c3)c3ccc(Cl)c(F)c32)nn1]

Dissolve 6-chloro-1-ethynyl-7-fluoro-3-(1-tetrahydropyran-2-ylpyrazol-4-yl)indole (190 mg, 0.525 mmol) in DMF (3 mL) and water (3 mL). Add copper (10 mg, 0.16 mmol), cupric sulfate (20 mg, 0.13 mmol) and 2-azidoethoxy-tert-butyl-dimethyl-silane (140 mg, 0.661 mmol). Purge with N₂. Stir at 110° C. for 0.5 hr. Combine with another batch run at 0.42×scale for work up and purification. Dilute with water, extract with EtOAc (3×), wash the combined organic layers with saturated aqueous NaCl, dry over anhydrous sodium sulfate, filter, and concentrate under vacuum. Purify by flash column chromatography eluting with EtOAc in petroleum ether to give tert-butyl-[2-[4-[6-chloro-7-fluoro-3-(1-tetrahydropyran-2-ylpyrazol-4-yl)indol-1-yl]triazol-1-yl]ethoxy]-dimethyl-silane (300 mg, 34%) as a brown oil. ES/MS (m/z): (³⁵Cl/³⁷Cl) 545.3/547.2 (M+H) and 461.2/463.1 (M+H-THP).

### EXAMPLE 218

### 2-[4-[6-Chloro-7-fluoro-3-(1H-pyrazol-4-yl)indol-1-yl]triazol-1-yl]ethanol

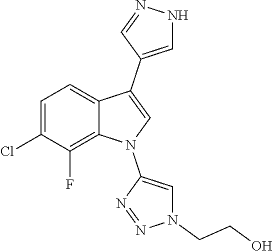 [SMILES: OCCn1cc(-n2cc(-c3cn[nH]c3)c3ccc(Cl)c(F)c32)nn1]

Suspend tert-butyl-[2-[4-[6-chloro-7-fluoro-3-(1-tetrahydropyran-2-ylpyrazol-4-yl)indol-1-yl]triazol-1-yl]ethoxy]-dimethyl-silane (300 mg, 0.253 mmol) in 4M HCl in MeOH (10 mL). Stir at ambient temperature for 2 hrs. Concentrate under vacuum and purify by prep-HPLC to give 2-[4-[6-chloro-7-fluoro-3-(1H-pyrazol-4-yl)indol-1-yl]triazol-1-yl]ethanol (85.6 mg, 97%) as a white solid. ES/MS (m/z): (³⁵Cl/³⁷Cl) 347.3/349.3 (M+H).

### Intermediate 528

### 6-Chloro-3-(1H-pyrazol-4-yl)-1H-indol-4-amine

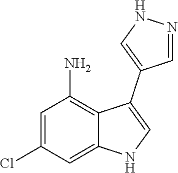 [SMILES: Nc1cc(Cl)cc2[nH]cc(-c3cn[nH]c3)c12]

Suspend tert-butyl N-[6-chloro-3-(1-tetrahydropyran-2-ylpyrazol-4-yl)-1H-indol-4-yl]carbamate (550 mg, 1.14 mmol) in 4M HCl in MeOH (5 mL) and stir at ambient temperature for 2.5 hrs. Filter and dry under vacuum to give 6-chloro-3-(1H-pyrazol-4-yl)-1H-indol-4-amine (290 mg, 91%, HCl salt) as a brown solid. ES/MS (m/z): (³⁵Cl/³⁷Cl) 233.0/235.0 (M+H).

### EXAMPLE 219

### N-(6-Chloro-3-(1H-pyrazol-4-yl)-1H-indol-4-yl)-2,2-difluoroacetamide

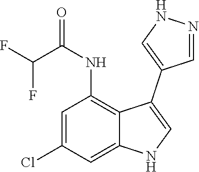 [SMILES: O=C(Nc1cc(Cl)cc2[nH]cc(-c3cn[nH]c3)c12)C(F)F]

Dissolve 6-chloro-3-(1H-pyrazol-4-yl)-1H-indol-4-amine (140 mg, 0.499 mmol, HCl salt), DIPEA (0.26 mL, 1.5 mmol) in DCM (3 mL). Add difluoroacetic anhydride (270 mg, 1.50 mmol) at 0° C. Stir at ambient temperature for 2 hrs. Dilute with DCM, wash with water and saturated aqueous NaCl, dry over anhydrous Na₂SO₄ and concentrate under vacuum. Purify by reverse phase prep-HPLC to give N-(6-chloro-3-(1H-pyrazol-4-yl)-1H-indol-4-yl)-2,2-difluoroacetamide (65.53 mg, 42%) as a white solid. ES/MS (m/z): (³⁵Cl/³⁷Cl) 311.2/313.2 (M+H).

### Intermediate 529

### 4-[tert-Butyl(dimethyl)silyl]oxy-2-[6,7-dichloro-1-(p-tolylsulfonyl)indol-4-yl]oxy-cyclopentanol

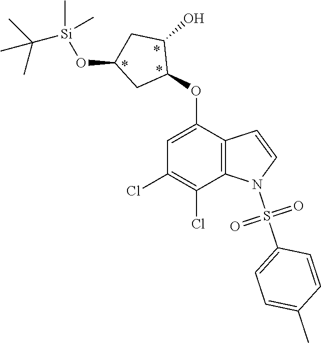 [SMILES: Cc1ccc(S(=O)(=O)n2ccc3c(O[C@H]4C[C@@H](O[Si](C)(C)C(C)(C)C)C[C@@H]4O)cc(Cl)c(Cl)c32)cc1]

Dissolve 6,7-dichloro-1-(p-tolylsulfonyl) indol-4-ol (500 mg, 1.26 mmol) in ethanol (5 mL). Add tert-butyl-dimethyl-[[trans-6-oxabicyclo[3.1.0]hexan-3-yl]oxy]silane (1.02 g, 3.81 mmol, 80% purity) and potassium carbonate (350 mg, 2.53 mmol), then stir at 100° C. for 16-18 hrs. Combine with another batch run at 0.10×scale for work up and purification. Filter and concentrate under vacuum. Purify by flash column chromatography eluting with EtOAc in petroleum ether to give 4-[tert-butyl(dimethyl)silyl]oxy -2-[6,7-dichloro-1-(p-tolylsulfonyl)indol-4-yl]oxy-cyclopentanol (418 mg, 42%) as a yellow oil. The material is a racemic mixture. ES/MS (m/z): (³⁵Cl/³⁷Cl) 570.1/572.0 (M+H).

### Intermediate 530

### 4-[(6,7-Dichloro-1H-indol-4-yl)oxy]cyclopentane-1,3-diol

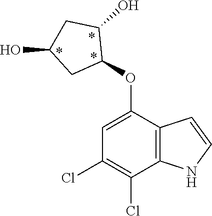 [SMILES: O[C@@H]1C[C@H](Oc2cc(Cl)c(Cl)c3[nH]ccc23)[C@@H](O)C1]

Dissolve 4-[tert-butyl(dimethyl)silyl]oxy-2-[6,7-dichloro-1-(p-tolylsulfonyl)indol-4-yl]oxy-cyclopentanol (418 mg, 0.586 mmol) in THF (2 mL) and add TBAF (4 mL, 1.0M in THF), then stir at ambient temperature for 2 days. Quench with saturated aqueous NH₄Cl, stir at ambient temperature for 15 minutes, dilute with water, separate layers, extract aqueous layer with EtOAc (3×), dry combined organic layers over anhydrous sodium sulfate, filter, and concentrate under vacuum. Purify by flash column chromatography eluting with MeOH in DCM to give 4-[(6,7-dichloro-1H-indol-4-yl)oxy]cyclopentane-1,3-diol (384 mg, 99+%) as a yellow oil. The material is a racemic mixture. ES/MS (m/z): (³⁵Cl/³⁷Cl) 301.9/303.9 (M+H).

### Intermediate 531

### 4-[(6,7-Dichloro-3-iodo-1H-indol-4-yl)oxy]cyclopentane-1,3-diol

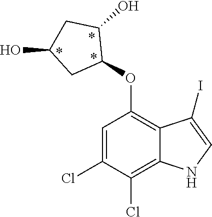 [SMILES: O[C@@H]1C[C@H](Oc2cc(Cl)c(Cl)c3[nH]cc(I)c23)[C@@H](O)C1]

Dissolve 4-[(6,7-dichloro-1H-indol-4-yl)oxy]cyclopentane-1,3-diol (354 mg, 1.13 mmol) in DMF (3 mL). Add NIS (310 mg, 1.35 mmol) and stir at ambient temperature for 1.5 hr. Quench with saturated aqueous Na₂SO₃ and dilute with EtOAc. Wash with water and extract the aqueous layer with EtOAc (3×). Wash the combined organic layers with saturated aqueous NaCl, dry over anhydrous sodium sulfate, filter, and concentrate under vacuum to give 4-[(6,7-dichloro-3-iodo-1H-indol-4-yl)oxy]cyclopentane-1,3-diol (235 mg, 37%) as a dark red oil. The material is a racemic mixture.

### Intermediate 532

### 4-[[6, 7-Dichloro-3-(1-tetrahydropyran-2-ylpyrazol-4-yl)-1H-indol-4-yl]oxy]cyclopentane-1,3-diol

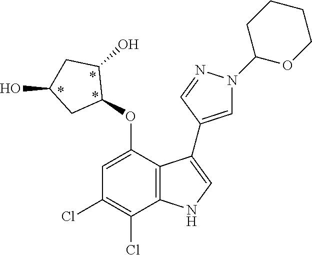 [SMILES: O[C@@H]1C[C@H](Oc2cc(Cl)c(Cl)c3[nH]cc(-c4cnn(C5CCCCO5)c4)c23)[C@@H](O)C1]

Suspend 4-[(6,7-dichloro-3-iodo-1H-indol-4-yl)oxy]cyclopentane-1,3-diol (235 mg, 0.412 mmol), 1-(tetrahydro-2H-pyran-2-yl)-4-(4,4,5,5-tetramethyl-1,3,2-dioxaborolan-2-yl)-1H-pyrazole (250 mg, 0.854 mmol), Pd(dtbpf)Cl₂ (57 mg, 0.086 mmol) and sodium carbonate (136 mg, 1.28 mmol) in 1,4-dioxane (4 mL) and water (1 mL). Purge with N₂ and heat at 90° C. for 2 hrs. Cool to ambient temperature and concentrate under vacuum. Purify by flash column chromatography eluting with MeOH in DCM to give 4-[[6,7-dichloro-3-(1-tetrahydropyran-2-ylpyrazol-4-yl)-1H-indol-4-yl]oxy]cyclopentane-1,3-diol (263 mg, 99+%) as a black solid. The material is a racemic mixture. ES/MS (m/z): (³⁵Cl/³⁷Cl) 452.0/454.0 (M+H).

### EXAMPLE 220

### 4-[[6,7-Dichloro-3-(1H-pyrazol-4-yl)-1H-indol-4-yl]oxy]cyclopentane-1,3-diol (racemic mixture)

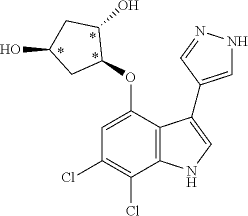 [SMILES: O[C@@H]1C[C@H](Oc2cc(Cl)c(Cl)c3[nH]cc(-c4cn[nH]c4)c23)[C@@H](O)C1]

Suspend 4-[[6,7-dichloro-3-(1-tetrahydropyran-2-ylpyrazol-4-yl)-1H-indol-4-yl]oxy]cyclopentane-1,3-diol (263 mg, 0.483 mmol) in THF (3 mL). Add 6M aqueous HCl (6 mL) and stir at ambient temperature for 2 hrs. Concentrate under vacuum and purify by prep-HPLC to give 4-[[6,7-dichloro-3-(1H-pyrazol-4-yl)-1H-indol-4-yl]oxy]cyclopentane-1,3-diol (50.91 mg, 29%) as a white solid. The material is a racemic mixture. ES/MS (m/z): (³⁵Cl/³⁷Cl) 368.3/370.3 (M+H).

### Intermediate 533

### 1-(Benzenesulfonyl)-4-bromo-6,7-dichloro-indole

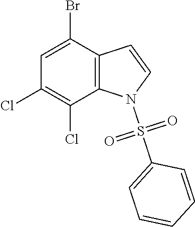 [SMILES: O=S(=O)(c1ccccc1)n1ccc2c(Br)cc(Cl)c(Cl)c21]

Dissolve 4-bromo-6,7-dichloro-1H-indole (20.0 g, 75.5 mmol) in THF (380 mL). Add NaHMDS (76 mL, 76 mmol, 1.0M in THF) at 0° C. Stir at 0° C. for 15 min. Add benzenesulfonyl chloride (10 mL, 76.6 mmol) dropwise at 0° C. Then stir at ambient temperature for 2 hrs. Quench with saturated aqueous sodium bicarbonate and water, extract with EtOAc, wash with 1:1 water/saturated aqueous sodium bicarbonate and then with saturated aqueous NaCl, dry over anhydrous sodium sulfate, filter, and concentrate under vacuum. Purify by trituration with petroleum ether/EtOAc (5/1) followed by filtration, washing with methanol and petroleum ether, and drying to give 1-(benzenesulfonyl)-4-bromo-6,7-dichloro-indole (22.3 g, 69%) as an off-white solid. ES/MS (m/z): 402.2/404.2/406.2 (M−H).

### Intermediate 534

### 1-(Benzenesulfonyl)-4-bromo-6, 7-dichloro-indole-2-carbaldehyde

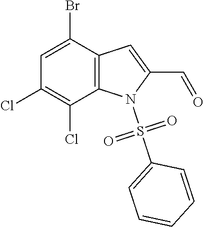 [SMILES: O=Cc1cc2c(Br)cc(Cl)c(Cl)c2n1S(=O)(=O)c1ccccc1]

Dissolve 1-(benzenesulfonyl)-4-bromo-6,7-dichloro-indole (20.0 g, 46.9 mmol) in THF (420 mL). Add freshly prepared LDA (116 mL, 59.2 mmol, 0.51M in THF) dropwise at −70° C. Stir at −70° C. for 30 min. Add DMF (6 mL, 77.6 mmol) dropwise at -70° C. and stir for 45 min. Quench with saturated aqueous NH₄Cl and water, extract with EtOAc, wash with saturated aqueous NaCl, dry over anhydrous sodium sulfate, filter, and concentrate under vacuum. Purify by trituration with MeOH to give 1-(benzenesulfonyl)-4-bromo-6,7-dichloro-indole-2-carbaldehyde (13 g, 61%) as a white solid. ES/MS (m/z): 431.4/433.6/435.4 (M+H) and 448.6/450.6/452.6 (M+NH₄).

### Intermediate 535

### (2E)-1-(Benzenesulfonyl)-4-bromo-6,7-dichloro-indole-2-carbaldehyde oxime

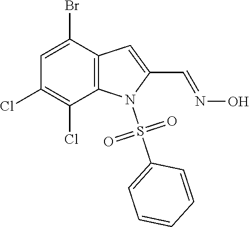 [SMILES: O=S(=O)(c1ccccc1)n1c(/C=N/O)cc2c(Br)cc(Cl)c(Cl)c21]

Dissolve 1-(benzenesulfonyl)-4-bromo-6,7-dichloro-indole-2-carbaldehyde (13.0 g, 28.5 mmol) in ethanol (300 mL) and water (100 mL). Add hydroxylamine hydrochloride (3.10 g, 44.6 mmol) and sodium carbonate (3.55 g, 33.5 mmol). Stir at 60° C. for 16-18 hrs. Dilute with water, extract with EtOAc (2×), wash the combined organic layers with saturated aqueous NaCl, dry over anhydrous magnesium sulfate, filter, and concentrate under vacuum to give (2E)-1-(benzenesulfonyl)-4-bromo-6,7-dichloro-indole-2-carbaldehyde oxime (15.6 g, 82%) as a light-yellow solid. ES/MS (m/z): 446.8/448.8/450.8 (M+H).

### Intermediate 536

### tert-Butyl N-[(4-bromo-6,7-dichloro-1H-indol-2-yl)methyl]carbamate

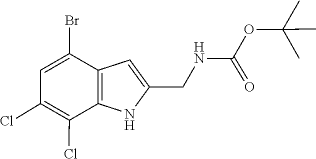 [SMILES: CC(C)(C)OC(=O)NCc1cc2c(Br)cc(Cl)c(Cl)c2[nH]1]

Dissolve (2E)-1-(benzenesulfonyl)-4-bromo-6,7-dichloro-indole-2-carbaldehyde oxime (15.6 g, 25.4 mmol), NiCl₂ (3.30 g, 25.5 mmol) and Boc₂O (20.0 mL, 90.7 mmol) in MeOH (250 mL). Add sodium borohydride (5.77 g, 153 mmol) in small portions at 0° C. Stir at ambient temperature for 2 hrs. Dilute with water, extract with EtOAc (2×), dry the combined organic layers over anhydrous magnesium sulfate, filter, and concentrate under vacuum. Purify by flash column chromatography eluting with EtOAc in petroleum ether to give tert-butyl N-[(4-bromo-6,7-dichloro-1H-indol-2-yl)methyl]carbamate (7.00 g, 63%) as a yellow solid.

### Intermediate 537

### tert-Butyl N-[[4-(tert-butoxycarbonylamino)-6,7-dichloro-1H-indol-2-yl]methyl]carbamate

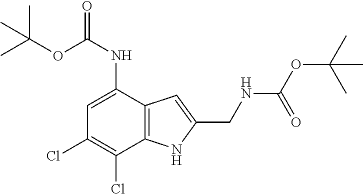 [SMILES: CC(C)(C)OC(=O)NCc1cc2c(NC(=O)OC(C)(C)C)cc(Cl)c(Cl)c2[nH]1]

Suspend tert-butyl N-[(4-bromo-6,7-dichloro-1H-indol-2-yl)methyl]carbamate (550 mg, 1.26 mmol), tert-butyl carbamate (250 mg, 2.13 mmol), Xantphos-Pd-G₂ (140 mg, 0.141 mmol) and Cs₂CO₃ (910 mg, 2.79 mmol) in 1,4-dioxane (50 mL). Purge with N₂ then stir at 100° C. for 16-18 hrs. Cool to ambient temperature and concentrate under vacuum. Purify by flash column chromatography eluting with EtOAc in petroleum ether to give tert-butyl N-[[4-(tert-butoxycarbonylamino)-6,7-dichloro-1H-indol-2-yl]methyl]carbamate (250 mg, 39%) as a yellow oil.

### Intermediate 538

### tert-Butyl N-[[3-bromo-4-(tert-butoxycarbonylamino)-6,7-dichloro-1H-indol-2-yl]methyl]carbamate

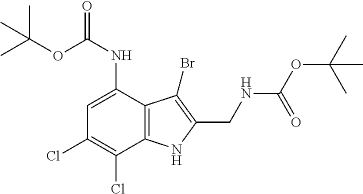 [SMILES: CC(C)(C)OC(=O)NCc1[nH]c2c(Cl)c(Cl)cc(NC(=O)OC(C)(C)C)c2c1Br]

Dissolve tert-butyl N-[[4-(tert-butoxycarbonylamino)-6,7-dichloro-1H-indol-2-yl]methyl]carbamate (2.91 g, 5.75 mmol) in DMF (30 mL) and add NBS (1.25 g, 7.02 mmol) at 0° C. Stir at 0° C. for 1.5 hr. Quench with saturated aqueous Na₂SO₃, extract with EtOAc (2×), wash the combined organic layers with saturated aqueous NaCl, dry over anhydrous sodium sulfate, filter, and concentrate under vacuum. Purify by flash column chromatography eluting with EtOAc in petroleum ether to give tert-butyl N-[[3-bromo-4-(tert-butoxycarbonylamino)-6,7-dichloro-1H-indol-2-yl]methyl]carbamate (1.30 g, 42%) as a light-yellow solid.

### Intermediate 539

### tert-Butyl N-[[4-(tert-butoxycarbonylamino)-6,7-dichloro-3-(1-tetrahydropyran-2-ylpyrazol-4-yl)-1H-indol-2-yl]methyl]carbamate

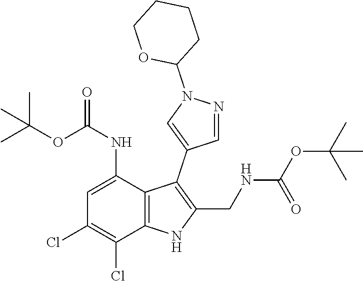 [SMILES: CC(C)(C)OC(=O)NCc1[nH]c2c(Cl)c(Cl)cc(NC(=O)OC(C)(C)C)c2c1-c1cnn(C2CCCCO2)c1]

Suspend tert-butyl N-[[3-bromo-4-(tert-butoxycarbonylamino)-6,7-dichloro-1H-indol-2-yl]methyl]carbamate (1.30 g, 2.30 mmol), 1-(tetrahydro-2H-pyran-2-yl)-4-(4,4,5,5-tetramethyl-1,3,2-dioxaborolan-2-yl)-1H-pyrazole (1.01 g, 3.45 mmol), Na₂CO₃ (490 mg, 4.62 mmol) and Pd(dppf)Cl₂ (180 mg, 0.234 mmol) in 1,4-dioxane (16 mL) and water (4 mL). Purge with N₂ and stir at 90° C. for 16-18 hrs. Cool to ambient temperature and concentrate under vacuum. Purify by flash column chromatography eluting with EtOAc in petroleum ether to give tert-butyl N-[[4-(tert-butoxycarbonylamino)-6,7-dichloro-3-(1-tetrahydropyran-2-ylpyrazol-4-yl)-1H-indol-2-yl]methyl]carbamate (630 mg, 46%) as a light-yellow solid. ES/MS (m/z): (³⁵Cl/³⁷Cl) 580.2/582.1 (M+H) and 602.2/604.2 (M+Na).

### Intermediate 540

### 2-(Aminomethyl)-6,7-dichloro-3-(1H-pyrazol-4-yl)-1H-indol-4-amine dihydrochloride

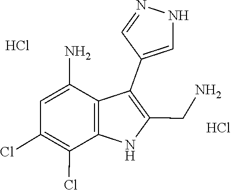 [SMILES: Cl ; NCc1[nH]c2c(Cl)c(Cl)cc(N)c2c1-c1cn[nH]c1 ; Cl]

Dissolve tert-butyl N-[[4-(tert-butoxycarbonylamino)-6,7-dichloro-3-(1-tetrahydropyran-2-ylpyrazol-4-yl)-1H-indol-2-yl]methyl]carbamate (630 mg, 1.05 mmol) in 4M HCl in MeOH (8 mL). Stir at ambient temperature for 1 hr. Concentrate under vacuum to give 2-(aminomethyl)-6,7-dichloro-3-(1H-pyrazol-4-yl)-1H-indol-4-amine dihydrochloride (400 mg, 82%) as a pink solid. ES/MS (m/z): (³⁵Cl/³⁷Cl) 296.0/297.6 (M+H) and (³⁵Cl/³⁷Cl) 279.0/280.8 (M+H—NH₃).

### EXAMPLE 221

### N-[[6,7-Dichloro-4-[(2-hydroxyacetyl)amino]-3-(1H-pyrazol-4-yl)-1H-indol-2-yl]methyl]-2-hydroxy-acetamide

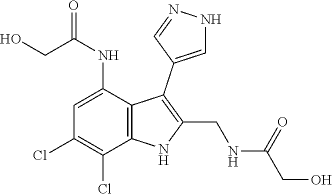 [SMILES: O=C(CO)NCc1[nH]c2c(Cl)c(Cl)cc(NC(=O)CO)c2c1-c1cn[nH]c1]

Dissolve 2-(aminomethyl)-6,7-dichloro-3-(1H-pyrazol-4-yl)-1H-indol-4-amine dihydrochloride (100 mg, 0.217 mmol), TEA (0.45 mL, 3.2 mmol) and DMAP (30 mg, 0.25 mmol) in DCM (5 mL). Add acetoxyacetyl chloride (0.25 mL, 2.3 mmol) at 0° C. and stir at ambient temperature for 16-18 hrs. Combine with another batch run at 0.10×scale for work up and purification. Quench with saturated aqueous sodium bicarbonate, extract with DCM, wash sequentially with water and saturated aqueous NaCl, dry over anhydrous sodium sulfate, filter, and concentrate under vacuum. Dissolve the residue in MeOH (3 mL) and add K₂CO₃ (300 mg, 2.17 mmol), then stir at ambient temperature for 1 hr. Dilute with water, extract with EtOAc, wash with saturated aqueous NaCl, dry over anhydrous sodium sulfate, filter, and concentrate under vacuum. Purify by prep-HPLC to give N-[[6, 7-dichloro-4-[(2-hydroxyacetyl)amino]-3-(1H-pyrazol-4-yl)-1H-indol-2-yl]methyl]-2-hydroxy-acetamide (44.65 mg, 44%) as a yellow solid. ES/MS (m/z): (³⁵Cl/³⁷Cl) 412.0/414.0 (M+H).

### Intermediate 541

### [2-[[4-Amino-6,7-dichloro-3-(1H-pyrazol-4-yl)-1H-indol-2-yl]methylamino]-2-oxo-ethyl] acetate

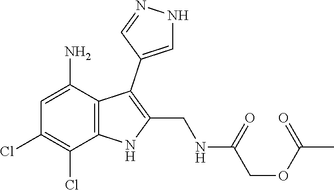 [SMILES: CC(=O)OCC(=O)NCc1[nH]c2c(Cl)c(Cl)cc(N)c2c1-c1cn[nH]c1]

Dissolve 2-(aminomethyl)-6,7-dichloro-3-(1H-pyrazol-4-yl)-1H-indol-4-amine hydrochloride (160 mg, 0.347 mmol) and TEA (0.73 mL, 5.20 mmol) in DCM (5 mL). Add acetoxyacetyl chloride (0.040 mL, 0.37 mmol) at 0° C., then stir at ambient temperature for 16-18 hrs. Quench with saturated aqueous sodium bicarbonate, extract with EtOAc (2×), wash the combined organic layers with saturated aqueous NaCl, dry over anhydrous sodium sulfate, filter, and concentrate under vacuum to give [2-[[4-amino-6,7-dichloro-3-(1H-pyrazol-4-yl)-1H-indol-2-yl]methylamino]-2-oxo-ethyl] acetate (120 mg, 58%) as a light-yellow oil. ES/MS (m/z): (³⁵Cl/³⁷Cl) 395.9/398.0 (M+H).

### EXAMPLE 222

### N-[[4-Acetamido-6,7-dichloro-3-(1H-pyrazol-4-yl)-1H-indol-2-yl]methyl]-2-hydroxy-acetamide

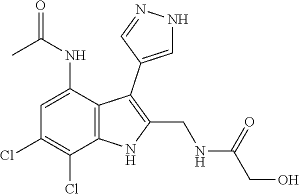 [SMILES: CC(=O)Nc1cc(Cl)c(Cl)c2[nH]c(CNC(=O)CO)c(-c3cn[nH]c3)c12]

Dissolve [2-[[4-amino-6,7-dichloro-3-(1H-pyrazol-4-yl)-1H-indol-2-yl]methylamino]-2-oxo-ethyl] acetate (110 mg, 0.183 mmol) and TEA (0.39 mL, 2.8 mmol) in DCM (3 mL). Add Ac₂O (0.18 mL, 1.9 mmol) at 0° C. and stir at ambient temperature for 16-18 hrs. Quench with saturated aqueous sodium bicarbonate, extract with EtOAc (2×), wash the combined organic layers with saturated aqueous NaCl, dry over anhydrous sodium sulfate, filter, and concentrate under vacuum. Dissolve the residue in MeOH (3 mL) and add K₂CO₃ (255 mg, 1.84 mmol), then stir at ambient temperature for 1 hr. Dilute with water, extract with EtOAc (2×), wash the combined organic layers with saturated aqueous NaCl, dry over anhydrous sodium sulfate, filter, and concentrate under vacuum. Purify by prep-HPLC to give N-[[4-acetamido-6,7-dichloro-3-(1H-pyrazol-4-yl)-1H-indol-2-yl]methyl]-2-hydroxy-acetamide (23.14 mg, 31%) as a white solid. ES/MS (m/z): (³⁵Cl/³⁷Cl) 396.0/397.9 (M+H).

### Intermediate 542

### [6,7-Dichloro-3-(1H-pyrazol-4-yl)-1H-indol-2-yl]methanamine hydrochloride

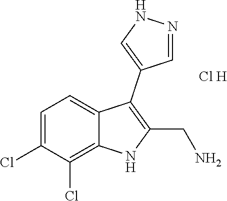

Dissolve [6,7-dichloro-3-(1-tetrahydropyran-2-ylpyrazol-4-yl)-1H-indol-2-yl]methanamine (200 mg, 0.493 mmol) in MeOH (0.25 mL) and add 4M HCl in MeOH (4 mL). Stir at ambient temperature for 16-18 hrs. Concentrate under vacuum and purify by trituration with CH₃CN to give [6,7-dichloro-3-(1H-pyrazol-4-yl)-1H-indol-2-yl]methanamine hydrochloride (160 mg, 97%) as a white solid. ES/MS (m/z): (³⁵Cl/³⁷Cl) 281.1/283.1 (M+H) and (³⁵Cl/³⁷Cl) 264.1/266.0 (M+H—NH₃).

### EXAMPLE 223

### N-[[6,7-Dichloro-3-(1H-pyrazol-4-yl)-1H-indol-2-yl]methyl]-3-hydroxy-oxetane-3-carboxamide

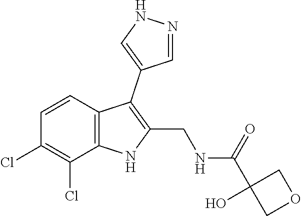 [SMILES: O=C(NCc1[nH]c2c(Cl)c(Cl)ccc2c1-c1cn[nH]c1)C1(O)COC1]

Dissolve 3-hydroxyoxetane-3-carboxylic acid (CAS No. 1450997-88-2) (60 mg, 0.49 mmol) and HATU (145 mg, 0.370 mmol) in DMF (3 mL). Add DIPEA (0.13 mL, 0.74 mmol) and stir at ambient temperature for 30 min. Add [6,7-dichloro-3-(1H-pyrazol-4-yl)-1H-indol-2-yl]methanamine hydrochloride (80 mg, 0.24 mmol) and stir at 60° C. for 2 days. Dilute with water, extract with EtOAc, wash the organic layer with water (5×) and saturated aqueous NaCl (2×), dry over sodium sulfate, filter, and concentrate under vacuum. Purify by prep-HPLC to give N-[[6,7-dichloro-3-(1H-pyrazol-4-yl)-1H-indol-2-yl]methyl]-3-hydroxy-oxetane-3-carboxamide (17.37 mg, 18%) as a yellow solid. ES/MS (m/z): 381.3/383.2 (M+H).

### EXAMPLE 224

### N-[[6,7-Dichloro-3-(1H-pyrazol-4-yl)-1H-indol-2-yl]methyl]oxetane-3-carboxamide

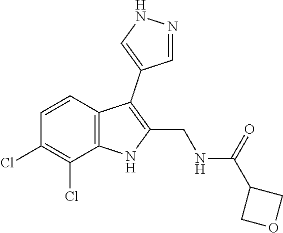 [SMILES: O=C(NCc1[nH]c2c(Cl)c(Cl)ccc2c1-c1cn[nH]c1)C1COC1]

Dissolve oxetane-3-carboxylic acid (CAS No. 114012-41-8) (50 mg, 0.49 mmol) and HATU (145 mg, 0.370 mmol) in DMF (2 mL). Add DIPEA (0.3 mL, 2 mmol) and stir at ambient temperature for 15 min. Add [6,7-dichloro-3-(1H-pyrazol-4-yl)-1H-indol-2-yl]methanamine hydrochloride (80 mg, 0.24 mmol) and stir at ambient temperature for 16-18 hrs. Dilute with water, extract with EtOAc, wash the organic layer with water (5×) and saturated aqueous NaCl (2×), dry over sodium sulfate, filter, and concentrate under vacuum. Purify by prep-HPLC to give N-[[6,7-dichloro-3-(1H-pyrazol-4-yl)-1H-indol-2-yl]methyl]oxetane-3-carboxamide (47.96 mg, 55%) as a white solid. ES/MS (m/z): (³⁵Cl/³⁷Cl) 365.3/367.3 (M+H).

### Intermediate 543

### Methyl 2-(4-bromo-6,7-dichloro-1H-indol-2-yl)acetate

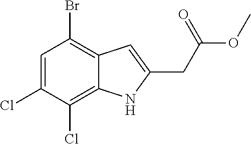 [SMILES: COC(=O)Cc1cc2c(Br)cc(Cl)c(Cl)c2[nH]1]

Dissolve 4-bromo-6,7-dichloro-1H-indole (3.00 g, 10.2 mmol), methyl bromoacetate (1.9 g, 12 mmol), Pd(PhCN)₂Cl₂ (820 mg, 2.03 mmol), norborn-2-ene (3.9 g, 41 mmol) and NaHCO₃ (3.4 g, 40 mmol) in water (0.28 mL) and DMF (30 mL). Purge with N₂ and heat to 70° C. for 16-18 hrs. Cool to ambient temperature, dilute with EtOAc, wash with water and saturated aqueous NaCl, dry over sodium sulfate, filter, and concentrate under vacuum. Purify by flash column chromatography eluting with EtOAc in petroleum ether to give methyl 2-(4-bromo-6,7-dichloro-1H-indol-2-yl)acetate (2.4 g, 63%) as a yellow solid. ES/MS (m/z): 336.0/337.9/339.9 (M+H).

### Intermediate 544

### 2-(4-Bromo-6,7-dichloro-indolin-2-yl)ethanol

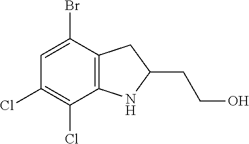 [SMILES: OCCC1Cc2c(Br)cc(Cl)c(Cl)c2N1]

Dissolve methyl 2-(4-bromo-6,7-dichloro-1H-indol-2-yl)acetate (2.40 g, 6.40 mmol) in DCM (40 mL). Add DIBAL-H (26 mL, 26.0 mmol, 1M in toluene) dropwise at −65° C. Stir at 0° C. for 1.5 hr. Dilute with DCM, quench with water (2.6 mL) and 5N aqueous NaOH (2.6 mL) at 0° C. Add water (7.6 mL) and stir for 15 min, filter, and concentrate under vacuum. Purify by flash column chromatography eluting with EtOAc in petroleum ether to give 2-(4-bromo-6,7-dichloro-indolin-2-yl)ethanol (1.50 g, 56%) as a brown solid. ES/MS (m/z): 310.0/312.0/314.0 (M+H).

### Intermediate 545

### 4-Bromo-6, 7-dichloro-2-(2-tetrahydropyran-2-yloxy ethyl)-1H-indole

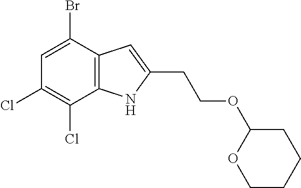 [SMILES: Clc1cc(Br)c2cc(CCOC3CCCCO3)[nH]c2c1Cl]

Dissolve 2-(4-bromo-6,7-dichloro-indolin-2-yl)ethanol (1.5 g, 3.6 mmol) and dihydropyran (0.45 mL, 4.9 mmol) in THF (15 mL). Add MeSO₃H (0.020 mL, 0.30 mmol) and stir at ambient temperature for 16-18 hrs. Dilute with water and saturated aqueous sodium bicarbonate and extract with EtOAc (2×). Wash the combined organic layers with saturated aqueous NaCl (2×), dry over sodium sulfate, filter, and concentrate under vacuum. Purify by flash column chromatography eluting with EtOAc in petroleum ether to give 4-bromo-6, 7-dichloro-2-(2-tetrahydropyran-2-yloxy ethyl)-1H-indole (1.5 g, 79%) as a yellow oil. ES/MS (m/z): 392.0/394.0/395.9 (M+H).

### Intermediate 546

### tert-Butyl N-[6,7-dichloro-2-(2-tetrahydropyran-2-yloxyethyl)-1H-indol-4-yl]carbamate

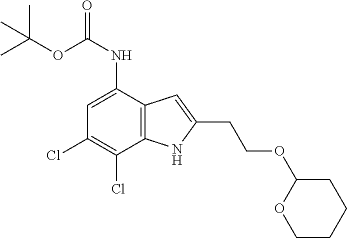 [SMILES: CC(C)(C)OC(=O)Nc1cc(Cl)c(Cl)c2[nH]c(CCOC3CCCCO3)cc12]

Dissolve 4-bromo-6,7-dichloro-2-(2-tetrahydropyran-2-yloxyethyl)-1H-indole (2.35 g, 4.48 mmol), tert-butyl carbamate (860 mg, 7.19 mmol), XantPhos-Pd-G₂ (445 mg, 0.448 mmol) and Cs₂CO₃ (4.38 g, 13.4 mmol) in 1,4-dioxane (50 mL). Purge with N₂ and stir at 100° C. for 16-18 hrs. Dilute with water, extract with EtOAc, wash with saturated aqueous NaCl, dry over sodium sulfate, filter, and concentrate under vacuum. Purify by flash column chromatography eluting with EtOAc in petroleum ether to give tert-butyl N-[6,7-dichloro-2-(2-tetrahydropyran-2-yl oxy ethyl)-1H-indol-4-yl] carbamate (1.9 g, 87%) as a yellow oil. ES/MS (m/z): (³⁵Cl/³⁷Cl) 429.2/431.1 (M+H) and 373.1/375.0 (M+H-tBu).

### Intermediate 547

### tert-Butyl N-[6,7-dichloro-3-iodo-2-(2-tetrahydropyran-2-yloxyethyl)-1H-indol-4-yl]carbamate

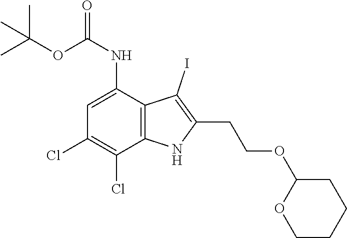 [SMILES: CC(C)(C)OC(=O)Nc1cc(Cl)c(Cl)c2[nH]c(CCOC3CCCCO3)c(I)c12]

Dissolve tert-butyl N-[6,7-dichloro-2-(2-tetrahydropyran-2-yloxyethyl)-1H-indol-4-yl]carbamate (1.90 g, 3.90 mmol) in DMF (15 mL). Add NIS (980 mg, 4.27 mmol) at 0° C. Stir at ambient temperature for 1 hr. Dilute with EtOAc, wash with water (2×) and saturated aqueous NaCl, dry over sodium sulfate, filter, and concentrate under vacuum. Purify by flash column chromatography eluting with EtOAc in petroleum ether to give tert-butyl N-[6,7-dichloro-3-iodo-2-(2-tetrahydropyran-2-yloxyethyl)-1H-indol-4-yl]carbamate (1.9 g, 83%) as a yellow oil. ES/MS (m/z): (³⁵Cl/³⁷Cl) 555.0/557.1 (M+H).

### Intermediate 548

### tert-Butyl N-[6,7-dichloro-2-(2-tetrahydropyran-2-yloxyethyl)-3-(1-tetrahydropyran-2-ylpyrazol-4-yl)-1H-indol-4-yl]carbamate

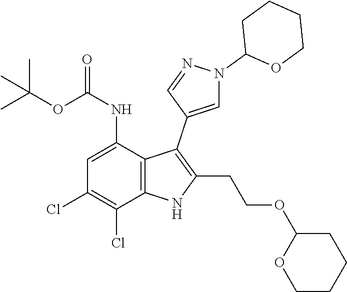 [SMILES: CC(C)(C)OC(=O)Nc1cc(Cl)c(Cl)c2[nH]c(CCOC3CCCCO3)c(-c3cnn(C4CCCCO4)c3)c12]

Dissolve tert-butyl N-[6,7-dichloro-3-iodo-2-(2-tetrahydropyran-2-yloxyethyl)-1H-indol-4-yl]carbamate (1.9 g, 3.3 mmol), 1-tetrahydropyran-2-yl-4-(4,4,5,5-tetramethyl-1,3,2-dioxaborolan-2-yl)pyrazole (1.4 g, 5.0 mmol), Pd(dtbpf)Cl₂ (440 mg, 0.662 mmol) and Na₂CO₃ (1.2 g, 11. mmol) in 1,4-dioxane (20 mL) and water (5 mL). Purge with N₂ and heat to 90° C. for 16-18 hrs. Cool to ambient temperature and concentrate under vacuum. Purify by flash column chromatography eluting with EtOAc in petroleum ether to give tert-butyl N-[6,7-dichloro-2-(2-tetrahydropyran-2-yloxyethyl)-3-(1-tetrahydropyran-2-ylpyrazol-4-yl)-1H-indol-4-yl]carbamate (1.5 g, 76%) as a yellow oil. ES/MS (m/z): (³⁵Cl/³⁷Cl) 579.2/581.2 (M+H).

### Intermediate 549

### 2-[4-Amino-6,7-dichloro-3-(1H-pyrazol-4-yl)-1H-indol-2-yl]ethanol

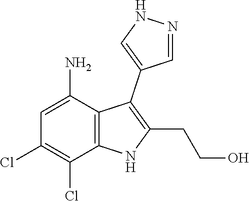 [SMILES: Nc1cc(Cl)c(Cl)c2[nH]c(CCO)c(-c3cn[nH]c3)c12]

Dissolve tert-butyl N-[6, 7-dichloro-2-(2-tetrahydropyran-2-yloxy ethyl)-3-(1-tetrahydropyran-2-ylpyrazol-4-yl)-1H-indol-4-yl]carbamate (750 mg, 1.23 mmol) in THF (4 mL). Add 6N aqueous HCl (8 mL). Stir at ambient temperature for 3 hrs. Filter and dry the precipitate under vacuum to give 2-[4-amino-6,7-dichloro-3-(1H-pyrazol-4-yl)-1H-indol-2-yl]ethanol (300 mg, 61%) as a white solid. ES/MS (m/z): (³⁵Cl/³⁷Cl) 310.8/312.8 (M+H).

### EXAMPLE 225

### N-[6,7-Dichloro-2-(2-hydroxyethyl)-3-(1H-pyrazol-4-yl)-1H-indol-4-yl]-2,2-difluoro-acetamide

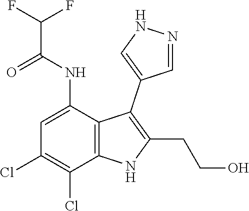 [SMILES: O=C(Nc1cc(Cl)c(Cl)c2[nH]c(CCO)c(-c3cn[nH]c3)c12)C(F)F]

Dissolve 2-[4-amino-6,7-dichloro-3-(1H-pyrazol-4-yl)-1H-indol-2-yl]ethanol (150 mg, 0.376 mmol), DIPEA (0.55 mL, 3.2 mmol) and DMAP (10 mg, 0.081 mmol) in DCM (3 mL). Add difluoroacetic anhydride (540 mg, 3.01 mmol) at 0° C. and stir at ambient temperature for 4 hrs. Dilute with water and saturated aqueous sodium bicarbonate and extract with DCM (2×). Wash the combined organic layers with saturated aqueous NaCl, dry over sodium sulfate, filter, and concentrate under vacuum. Dissolve the residue in MeOH (3 mL) and add K₂CO₃ (520 mg, 3.76 mmol) at 0° C. Stir at ambient temperature for 16-18 hrs. Dilute with water, extract with EtOAc (2×), wash with saturated aqueous NaCl, dry over sodium sulfate, filter, and concentrate under vacuum. Purify by prep-HPLC to give N-[6,7-dichloro-2-(2-hydroxyethyl)-3-(1H-pyrazol-4-yl)-1H-indol-4-yl]-2,2-difluoro-acetamide (42.14 mg, 29%) as a white solid. ES/MS (m/z): (³⁵Cl/³⁷Cl) 389.2/391.2 (M+H).

### Intermediate 550

### 2-[6-Chloro-3-(1-tetrahydropyran-2-ylpyrazol-4-yl)indol-1-yl]ethynyl-triisopropyl-silane

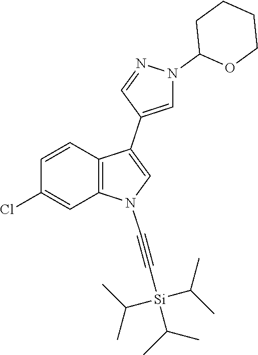 [SMILES: CC(C)[Si](C#Cn1cc(-c2cnn(C3CCCCO3)c2)c2ccc(Cl)cc21)(C(C)C)C(C)C]

Dissolve 6-chloro-3-(1-tetrahydropyran-2-ylpyrazol-4-yl)-1H-indole (500 mg, 1.59 mmol, prepared via the same route as 6-chloro-7-fluoro-3-(1-tetrahydropyran-2-ylpyrazol-4-yl)-1H-indole; Intermediate 524) in 1,4-dioxane (15 mL). Add (bromoethynyl)triisopropylsilane (2.14 g, 1.59 mmol), PEG-400 (150 mg), CuI (30 mg, 0.16 mmol) and Cs₂CO₃ (620 mg, 1.90 mmol). Purge with N₂ and stir at 130° C. for 16 hrs. Dilute with water, extract with EtOAc (3×), dry the combined organic layers over anhydrous sodium sulfate, filter, and concentrate under vacuum. Purify by flash column chromatography eluting with EtOAc in petroleum ether to give 2-[6-chloro-3-(1-tetrahydropyran-2-ylpyrazol-4-yl)indol-1-yl]ethynyl-triisopropyl-silane (250 mg, 32%) as a yellow oil.

### Intermediate 551

### 6-Chloro-1-ethynyl-3-(1-tetrahydropyran-2-ylpyrazol-4-yl)indole

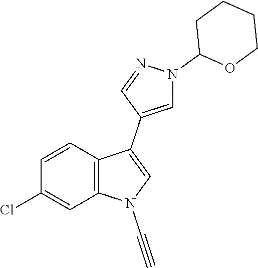 [SMILES: C#Cn1cc(-c2cnn(C3CCCCO3)c2)c2ccc(Cl)cc21]

Dissolve 2-[6-chloro-3-(1-tetrahydropyran-2-ylpyrazol-4-yl)indol-1-yl]ethynyl-triisopropyl-silane (250 mg, 0.508 mmol) in THF (10 mL) and add TBAF (5 mL, 1.0M in THF). Stir at ambient temperature for 2 hrs. Combine with another batch run at 0.96×scale for work up and purification. Dilute with EtOAc, wash with saturated aqueous NH₄Cl, dry over anhydrous sodium sulfate, filter, and concentrate under vacuum. Purify by flash column chromatography eluting with EtOAc in petroleum ether to give 6-chloro-1-ethynyl-3-(1-tetrahydropyran-2-ylpyrazol-4-yl)indole (290 mg, 85%) as a yellow oil. ES/MS (m/z): (³⁵Cl/³⁷Cl) 325.9/327.9 (M+H).

### Intermediate 552

### 6-Chloro-3-(1-tetrahydropyran-2-ylpyrazol-4-yl)-1-(1H-triazol-4-yl)indole

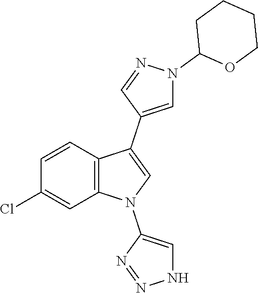 [SMILES: Clc1ccc2c(-c3cnn(C4CCCCO4)c3)cn(-c3c[nH]nn3)c2c1]

Dissolve 6-chloro-1-ethynyl-3-(1-tetrahydropyran-2-ylpyrazol-4-yl)indole (250 mg, 0.729 mmol) in DMF (5 mL) and MeOH (5 mL). Add TMSN₃ (110 mg, 0.955 mmol) and CuI (15 mg, 0.079 mmol). Purge with N₂ and stir at 100° C. for 6 hrs. Dilute with water, extract with EtOAc (3×), dry the combined organic layers over anhydrous sodium sulfate, filter, and concentrate under vacuum. Purify by flash column chromatography eluting with EtOAc in petroleum ether to give 6-chloro-3-(1-tetrahydropyran-2-ylpyrazol-4-yl)-1-(1H-triazol-4-yl)indole (168 mg, 56%) as a white solid. ES/MS (m/z): (³⁵Cl/³⁷Cl) 368.9/370.8 (M+H).

### EXAMPLE 226

### 6-Chloro-3-(1H-pyrazol-4-yl)-1-(1H-triazol-4-yl)indole

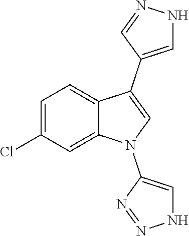 [SMILES: Clc1ccc2c(-c3cn[nH]c3)cn(-c3c[nH]nn3)c2c1]

Suspend 6-chloro-3-(1-tetrahydropyran-2-ylpyrazol-4-yl)-1-(1H-triazol-4-yl)indole (168 mg, 0.410 mmol) in 4M HCl in MeOH (5 mL) and stir at ambient temperature for 2 hrs. Concentrate under vacuum and purify by reverse phase prep-HPLC and then SFC to give 6-chloro-3-(1H-pyrazol-4-yl)-1-(1H-triazol-4-yl)indole (41.0 mg, 34%) as a white solid. ES/MS (m/z): (³⁵Cl/³⁷Cl) 285.3/287.2 (M+H).

### Intermediate 553

### Ethyl 2-[4-[6-chloro-3-(1-tetrahydropyran-2-ylpyrazol-4-yl)indol-1-yl]triazol-1-yl]acetate

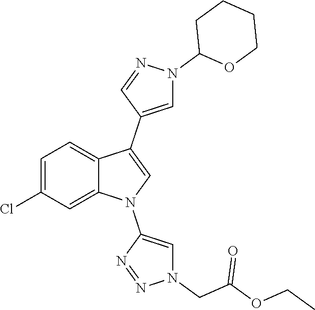 [SMILES: CCOC(=O)Cn1cc(-n2cc(-c3cnn(C4CCCCO4)c3)c3ccc(Cl)cc32)nn1]

Dissolve 6-chloro-1-ethynyl-3-(1-tetrahydropyran-2-ylpyrazol-4-yl)indole (640 mg, 1.96 mmol) in DMF (10 mL) and H₂O (10 mL). Add copper (25 mg, 0.39 mmol), cupric sulfate (63 mg, 0.39 mmol) and ethyl azidoacetate (310 mg, 2.35 mmol). Purge with N₂. Stir at 110° C. for 0.5 hr. Dilute with water, extract with EtOAc (3×), dry the combined organic layers over anhydrous sodium sulfate, filter, and concentrate under vacuum. Purify by flash column chromatography eluting with EtOAc in petroleum ether to give ethyl 2-[4-[6-chloro-3-(1-tetrahydropyran-2-ylpyrazol-4-yl)indol-1-yl]triazol-1-yl]acetate (290 mg, 16%) as a white solid. ES/MS (m/z): (³⁵Cl/³⁷Cl) 455.2/457.1 (M+H).

### Intermediate 554

### Methyl 2-[4-[6-chloro-3-(1H-pyrazol-4-yl)indol-1-yl]triazol-1-yl]acetate

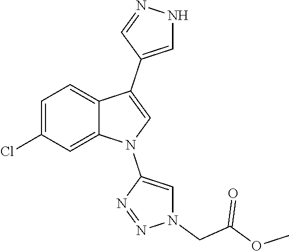 [SMILES: COC(=O)Cn1cc(-n2cc(-c3cn[nH]c3)c3ccc(Cl)cc32)nn1]

Dissolve ethyl 2-[4-[6-chloro-3-(1-tetrahydropyran-2-ylpyrazol-4-yl)indol-1-yl]triazol-1-yl]acetate (290 mg, 0.574 mmol) in 4M HCl in MeOH (5 mL) at ambient temperature. Stir at ambient temperature for 2 hrs. Concentrate under vacuum to give methyl 2-[4-[6-chloro-3-(1H-pyrazol-4-yl)indol-1-yl]triazol-1-yl]acetate (250 mg, 93%) as a white solid. ES/MS (m/z): (³⁵Cl/³⁷Cl) 357.1/359.0 (M+H).

### Intermediate 555

### 2-[4-[6-Chloro-3-(1H-pyrazol-4-yl)indol-1-yl]triazol-1-yl]acetic acid

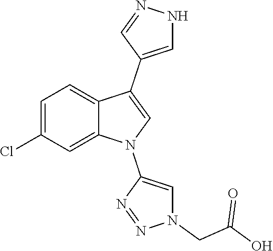 [SMILES: O=C(O)Cn1cc(-n2cc(-c3cn[nH]c3)c3ccc(Cl)cc32)nn1]

Dissolve methyl 2-[4-[6-chloro-3-(1H-pyrazol-4-yl)indol-1-yl]triazol-1-yl]acetate (250 mg, 0.533 mmol) in THF (5 mL) and H₂O (3 mL). Add sodium hydroxide (100 mg, 2.50 mmol). Stir at ambient temperature for 2 hrs. Dilute with water, adjust to pH=1 with 2N aqueous HCl, extract with EtOAc, wash with saturated aqueous NaCl, dry over anhydrous Na₂SO₄ and concentrate to give 2-[4-[6-chloro-3-(1H-pyrazol-4-yl)indol-1-yl]triazol-1-yl]acetic acid (171 mg, 84%) as a white solid. ES/MS (m/z): (³⁵Cl/³⁷Cl) 343.1/345.1 (M+H).

### EXAMPLE 227

### 2-[4-[6-Chloro-3-(1H-pyrazol-4-yl)indol-1-yl]triazol-1-yl]acetamide

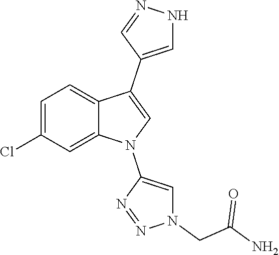 [SMILES: NC(=O)Cn1cc(-n2cc(-c3cn[nH]c3)c3ccc(Cl)cc32)nn1]

Dissolve 2-[4-[6-chloro-3-(1H-pyrazol-4-yl)indol-1-yl]triazol-1-yl]acetic acid (171 mg, 0.499 mmol), ammonium chloride (30 mg, 0.57 mmol), DIPEA (190 mg, 1.47 mmol) and HATU (220 mg, 0.567 mmol) in DMF (5 mL) at ambient temperature. Stir at ambient temperature for 2 hrs. Dilute with water, extract with EtOAc, wash with saturated aqueous NaCl, dry over with anhydrous Na₂SO₄ and concentrate under vacuum. Purify by flash column chromatography eluting with MeOH in DCM, then further purify by prep-HPLC to give 2-[4-[6-chloro-3-(1H-pyrazol-4-yl)indol-1-yl]triazol-1-yl]acetamide (27.45 mg, 16%) as a white solid. ES/MS (m/z): (³⁵Cl/³⁷Cl) 342.0/344.0 (M+H).

### Intermediate 556

### 2-[2-[4-[6-Chloro-3-(1-tetrahydropyran-2-ylpyrazol-4-yl) indol-1-yl] triazol-1-yl]ethyl] isoindoline-1,3-dione

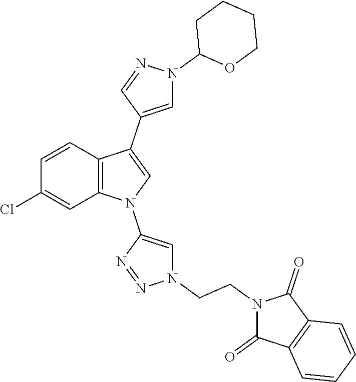 [SMILES: O=C1c2ccccc2C(=O)N1CCn1cc(-n2cc(-c3cnn(C4CCCCO4)c3)c3ccc(Cl)cc32)nn1]

Dissolve 6-chloro-1-ethynyl-3-(1-tetrahydropyran-2-ylpyrazol-4-yl)indole (538 mg, 1.65 mmol) in DMF (10 mL) and H₂O (10 mL). Add copper (21 mg, 0.33 mmol), cupric sulfate (52 mg, 0.33 mmol) and 2-(2-azidoethyl)isoindoline-1,3-dione (1.74 g, 7.25 mmol, 90% purity). Purge with N₂. Stir at 110° C. for 0.5 hr. Dilute with water, extract with EtOAc (3×), dry the combined organic layers over anhydrous sodium sulfate, filter, and concentrate under vacuum. Purify by flash column chromatography eluting with EtOAc in petroleum ether to give 2-[2-[4-[6-chloro-3-(1-tetrahydropyran-2-ylpyrazol-4-yl) indol-1-yl] triazol-1-yl]ethyl] isoindoline-1,3-dione (634 mg, 50%) as a white solid.

### Intermediate 557

### 2-[4-[6-Chloro-3-(1-tetrahydropyran-2-ylpyrazol-4-yl) indol-1-yl] triazol-1-yl] ethanamine

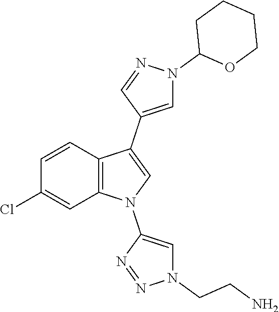 [SMILES: NCCn1cc(-n2cc(-c3cnn(C4CCCCO4)c3)c3ccc(Cl)cc32)nn1]

Dissolve 2-[2-[4-[6-chloro-3-(1-tetrahydropyran-2-ylpyrazol-4-yl) indol-1-yl] triazol-1-yl]ethyl] isoindoline-1,3-dione (620 mg, 1.03 mmol) in ethanol (5 mL) and add hydrazine hydrate (250 mg, 3.09 mmol, 43% purity). Stir at 95° C. for 5 hrs. Filter and concentrate the filtrate under vacuum. Dilute with water and collect the solids by vacuum filtration. Wash with water and dry to give 2-[4-[6-chloro-3-(1-tetrahydropyran-2-ylpyrazol-4-yl) indol-1-yl] triazol-1-yl] ethanamine (600 mg, 55%) as a yellow solid. ES/MS (m/z): (³⁵Cl/³⁷Cl) 412.1/414.1 (M+H).

### EXAMPLE 228

### 2-[4-[6-Chloro-3-(1H-pyrazol-4-yl) indol-1-yl] triazol-1-yl] ethanamine formic acid salt

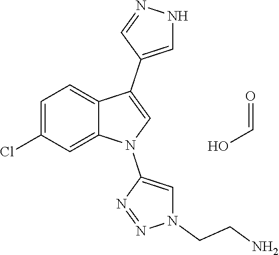 [SMILES: NCCn1cc(-n2cc(-c3cn[nH]c3)c3ccc(Cl)cc32)nn1 ; O=CO]

Dissolve 2-[4-[6-chloro-3-(1-tetrahydropyran-2-ylpyrazol-4-yl) indol-1-yl] triazol-1-yl] ethanamine (600 mg, 1.02 mmol, 70% purity) in 4M HCl in MeOH (8 mL). Stir at ambient temperature for 2 hrs. Concentrate under vacuum and purify by prep-HPLC to give 2-[4-[6-chloro-3-(1H-pyrazol-4-yl) indol-1-yl] triazol-1-yl] ethanamine formic acid salt (78.24 mg, 20%) as a white solid. ES/MS (m/z): (³⁵Cl/³⁷Cl) 328.1/330.1 (M+H).

### Intermediate 558

### tert-Butyl-[2-[4-[6-chloro-3-(1-tetrahydropyran-2-ylpyrazol-4-yl)indol-1-yl]triazol-1-yl]ethoxy]-dimethyl-silane

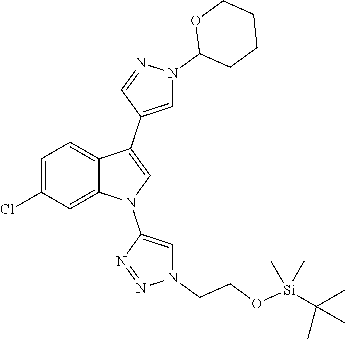 [SMILES: CC(C)(C)[Si](C)(C)OCCn1cc(-n2cc(-c3cnn(C4CCCCO4)c3)c3ccc(Cl)cc32)nn1]

Dissolve 6,7-dichloro-3-(1-tetrahydropyran-2-ylpyrazol-4-yl)-1H-indole (320 mg, 0.904 mmol), 2-(4-bromotriazol-1-yl)ethoxy-tert-butyl-dimethyl-silane (590 mg, 1.83 mmol), K₃PO₄ (₃95 mg, 1.82 mmol), DMEDA (81 mg, 0.92 mmol) and CuI (123 mg, 0.646 mmol) in toluene (5 mL). Purge with N₂ and stir at 110° C. for 16-18 hrs. Cool to ambient temperature, dilute with water, extract with EtOAc, wash with saturated aqueous NaCl, dry over anhydrous sodium sulfate, filter, and concentrate under vacuum. Purify by flash column chromatography eluting with EtOAc in petroleum ether to give tert-butyl-[2-[4-[6-chloro-3-(1-tetrahydropyran-2-ylpyrazol-4-yl)indol-1-yl]triazol-1-yl]ethoxy]-dimethyl-silane (240 mg, 20%) as a light-yellow oil. ES/MS (m/z): (³⁵Cl/³⁷Cl) 527.2/529.2 (M+H).

### EXAMPLE 229

### 2-[4-[6-Chloro-3-(1H-pyrazol-4-yl)indol-1-yl]triazol-2-yl] ethanol

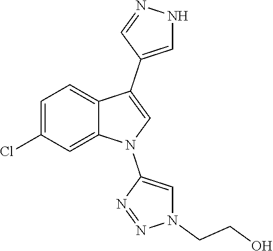 [SMILES: OCCn1cc(-n2cc(-c3cn[nH]c3)c3ccc(Cl)cc32)nn1]

Dissolve tert-butyl-[2-[4-[6-chloro-3-(1-tetrahydropyran-2-ylpyrazol-4-yl)indol -1-yl]triazol-1-yl]ethoxy]-dimethyl-silane (240 mg, 0.112 mmol, 51% purity) in THF (2 mL) and add 6M aqueous HCl (2 mL). Stir at ambient temperature for 1 hr. Concentrate under vacuum and purify by prep-HPLC, then SFC separation and further purify by prep-TLC (DCM/MeOH=10/1) to give 2-[4-[6-chloro-3-(1H-pyrazol-4-yl)indol-1-yl]triazol-2-yl]ethanol (15.67 mg, 26%) as a white solid. ES/MS (m/z): (³⁵Cl/³⁷Cl) 329.3/331.3 (M+H).

### Intermediate 559

### 6-Chloro-1-(2-methyltriazol-4-yl)-3-(1-tetrahydropyran-2-ylpyrazol-4-yl)indole

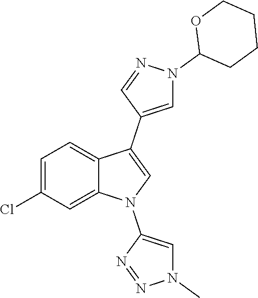 [SMILES: Cn1cc(-n2cc(-c3cnn(C4CCCCO4)c3)c3ccc(Cl)cc32)nn1]

Dissolve 6-chloro-3-(1-tetrahydropyran-2-ylpyrazol-4-yl)-1-(1H-triazol-4-yl)indole (220 mg, 0.537 mmol) in DMF (4 mL). Add potassium carbonate (150 mg, 1.09 mmol) and MeI (170 μL, 2.73 mmol). Stir at ambient temperature for 2 hrs. Dilute with water, extract with EtOAc (3×), wash with saturated aqueous NaCl, dry over anhydrous sodium sulfate, filter, and concentrate under vacuum. Purify by flash column chromatography eluting with EtOAc in petroleum ether to give 6-chloro-1-(2-methyltriazol-4-yl)-3-(1-tetrahydropyran-2-ylpyrazol-4-yl)indole (60 mg, 26%) as a yellow solid. ES/MS (m/z): (³⁵Cl/³⁷Cl) 299.0/300.9 (M+H) and 383.0/384.9 (M+H-THP).

### EXAMPLE 230

### 6-Chloro-1-(1-methyltriazol-4-yl)-3-(1H-pyrazol-4-yl)indole

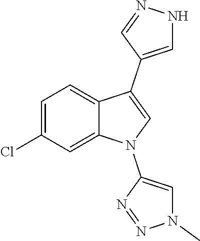 [SMILES: Cn1cc(-n2cc(-c3cn[nH]c3)c3ccc(Cl)cc32)nn1]

Suspend 6-chloro-1-(2-methyltriazol-4-yl)-3-(1-tetrahydropyran-2-ylpyrazol-4-yl)indole (60 mg, 0.14 mmol) and add 4M HCl in MeOH (5 mL). Stir at ambient temperature for 2 hrs. Combine with another batch run at 0.62×scale for work up and purification. Concentrate under vacuum and purify by prep-HPLC to give 6-chloro-1-(1-methyltriazol-4-yl)-3-(1H-pyrazol-4-yl)indole (27.6 mg, 40%) as a white solid. ES/MS (m/z): (³⁵Cl/³⁷Cl) 299.3/301.3 (M+H).

### EXAMPLE 231

### N-[6,7-Dichloro-3-(1H-pyrazol-4-yl)-1H-indol-4-yl]thioacetamide

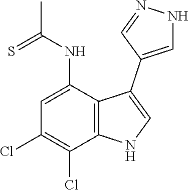 [SMILES: CC(=S)Nc1cc(Cl)c(Cl)c2[nH]cc(-c3cn[nH]c3)c12]

Add P₄S₁₀-pyridine complex (50 mg, 0.40 mmol) to a solution of N-[6,7-dichloro-3-(1H-pyrazol-4-yl)-1H-indol-4-yl]acetamide (100 mg, 0.232 mmol) in ACN (3 mL) in a microwave vial. Heat the reaction to 110° C. under microwave irradiation for 1 hr. Add additional P₄S₁₀-pyridine complex (50 mg, 0.40 mmol) and heat to 110° C. under microwave irradiation for 1 hr. Cool to ambient temperature and partially concentrate under vacuum to remove most of the ACN. Filter and concentrate the filtrate under vacuum to give a residue. Purify by reverse phase chromatography to give N-[6,7-dichloro-3-(1H-pyrazol-4-yl)-1H-indol-4-yl]thioacetamide (41 mg, 39%). ES/MS (m/z): (³⁵Cl/³⁷Cl) 325.0/327.0 (M+H). ¹H NMR (DMSO-d6): 12.76 (br s, 1H), 11.88 (br d, J=1.8 Hz, 1H), 11.32 (br s, 1H), 7.70 (br s, 1H), 7.56 (br s, 1H), 7.46 (d, J=2.5 Hz, 1H), 7.11 (s, 1H), 2.36 (s, 3H).

### Intermediate 560

### (R,E)-N-[3-[tert-butyl(dimethyl) silyl] oxypropylidene]-2-methyl-propane-2-sulfinamide

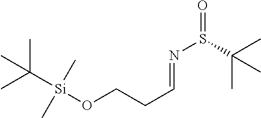 [SMILES: CC(C)(C)[S@@](=O)/N=C/CCO[Si](C)(C)C(C)(C)C]

Add titanium (IV) ethoxide (100 g, 0.416 mol) to 3-[(tert-butyldimethylsilyl)oxy]-1-propanal (62 g, 0.319 mol) in THF (750 mL) with stirring, under N₂ and at ambient temperature. Add (R)-(+)-2-methyl-2-propanesulfinamide (22 g, 0.176 mol) and allow the reaction to continue stirring at ambient temperature. After 1.5 hrs., carefully pour the reaction into a flask of vigorously stirred saturated aqueous NaCl. Dilute with EtOAc and decant the organics from the biphasic solution. Filter the remaining biphasic solution over diatomaceous earth and transfer the filtrate to a separatory funnel. Separate the layers and extract the aqueous with EtOAc. Wash the combined organics with saturated aqueous NaCl, dry over MgSO₄, filter and concentrate under vacuum. Purify the material via flash chromatography eluting with EtOAc in hexanes to give (R,E)-N-[3-[tert-butyl(dimethyl) silyl] oxypropylidene]-2-methyl-propane-2-sulfinamide (66.2 g, 68%).

### Intermediate 561

### (R)—N-[(1S)-1-[1-(Benzenesulfonyl)-4-bromo-6,7-dichloro-indol-2-yl]-3-[tert-butyl(dimethyl)silyl]oxy-propyl]-2-methyl-propane-2-sulfinamide

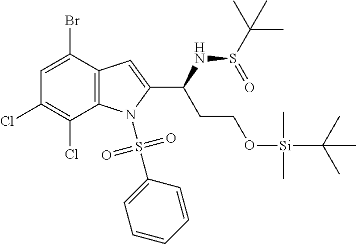 [SMILES: CC(C)(C)[S@@](=O)N[C@@H](CCO[Si](C)(C)C(C)(C)C)c1cc2c(Br)cc(Cl)c(Cl)c2n1S(=O)(=O)c1ccccc1]

Add LDA (2.0M in THF/heptane/ethylbenzene, 100 mL, 0.2 mol) slowly to 1-(benzenesulfonyl)-4-bromo-6,7-dichloro-indole (60 g, 0.148 mol) in THF (500 mL) with stirring, under nitrogen and at −78° C. After 40 min, add (R,E)-N-[3-[tert-butyl(dimethyl) silyl] oxypropylidene]-2-methyl-propane-2-sulfinamide (65 g, 0.22 mol) in THF (100 mL) slowly and allow the reaction to continue stirring with gradual warming to −20° C. After 3-4 hrs., carefully pour the reaction into saturated aqueous NH₄Cl and extract with EtOAc. Wash the combined organics with water and saturated aqueous NaCl. Dry the material over MgSO₄, filter and concentrate under vacuum. Remove unreacted starting material by trituration with diethyl ether and filtration. Concentrate the filtrate under vacuum. Purify the material by flash chromatography eluting with EtOAc in hexanes to give (R)—N-[(1S)-1-[1-(benzenesulfonyl)-4-bromo-6,7-dichloro-indol-2-yl]-3-[tert-butyl(dimethyl)silyl]oxy-propyl]-2-methyl-propane-2-sulfinamide (77.3 g, 75%). ES/MS m/z: 695.8/697.6 (M+H).

### Intermediate 562

### (R)—N—((S)-8-Bromo-5,6-dichloro-2,3-dihydro-1H-pyrrolo[1,2-a]indol-1-yl)-2-methylpropane-2-sulfinamide

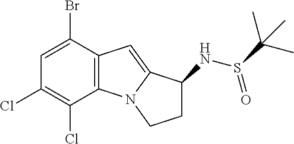 [SMILES: CC(C)(C)[S@@](=O)N[C@H]1CCn2c1cc1c(Br)cc(Cl)c(Cl)c12]

Add TBAF (1.0M in THF, 1.85 mL, 1.85 mmol) to (R)—N-[(1S)-1-[1-(benzenesulfonyl)-4-bromo-6,7-dichloro-indol-2-yl]-3-[tert-butyl(dimethyl)silyl]oxy-propyl]-2-methyl-propane-2-sulfinamide (2.15 g, 3.1 mmol) in 1,4-dioxane (15 mL) with stirring, under nitrogen and at 0° C. Allow the slurry to stir at 0° C. for 30 min. Add Cs₂CO₃ (1.1 g, 3.4 mmol) and tetrabutylammonium bromide (0.05 g, 0.15 mmol) to the cold reaction. Heat the reaction to 80° C. for 16-18 hrs. After 18 hrs., cool the reaction to ambient temperature, pour into saturated aqueous NH₄Cl and extract with EtOAc. Wash the combined organics with saturated aqueous NaCl, dry over MgSO₄, filter and concentrate under vacuum. Purify the residue via flash chromatography eluting with EtOAc in hexanes to give (R)—N—((S)-8-bromo-5,6-dichloro-2,3-dihydro-1H-pyrrolo[1,2-a]indol-1-yl)-2-methylpropane-2-sulfinamide (647 mg, 49%). ES/MS (m/z): 422.6/424.6 (M−H). ¹H NMR (DMSO-d6): 7.50 (s, 1H), 6.29 (d, 1H), 5.93 (d, 1H), 4.98-4.92 (m, 1H), 4.66-4.60 (m, 1H), 4.41 (dt, 1H), 2.95-2.88 (m, 1H), 2.52-2.58 (m, 1H), 1.16 (s, 9H).

### Intermediate 563

### (S)-8-Bromo-5,6-dichloro-2,3-dihydro-1H-pyrrolo[1,2-a]indol-1-amine hydrochloride

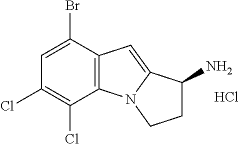 [SMILES: N[C@H]1CCn2c1cc1c(Br)cc(Cl)c(Cl)c12 ; Cl]

Add HCl (4M) in dioxane (1.3 mL, 5.2 mmol) to (R)—N—((S)-8-bromo-5,6-dichloro-2,3-dihydro-1H-pyrrolo[1,2-a]indol-1-yl)-2-methylpropane-2-sulfinamide (222 mg, 0.52 mmol) in 1,4-dioxane (3 mL) and MeOH (1 mL) with stirring, under nitrogen and at ambient temperature. After 1 hr., concentrate the reaction under vacuum to give (S)-8-bromo-5,6-dichloro-2,3-dihydro-1H-pyrrolo[1,2-a]indol-1-amine hydrochloride (191 mg, 99+%). ES/MS (m/z): 320.6/322.6 (M+H).

### Intermediate 564

### (S)—N-(8-Bromo-5,6-dichloro-2,3-dihydro-1H-pyrrolo[1,2-a]indol-1-yl)acetamide

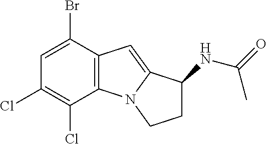 [SMILES: CC(=O)N[C@H]1CCn2c1cc1c(Br)cc(Cl)c(Cl)c12]

Add acetyl chloride (100 μL, 1.36 mmol) to (S)-8-bromo-5,6-dichloro-2,3-dihydro-1H-pyrrolo[1,2-a]indol-1-amine hydrochloride (405 mg, 1.13 mmol) and TEA (475 μL, 3.4 mmol) in THF (6 mL) with stirring, under nitrogen and at ambient temperature. After 45 min, pour the reaction into water and extract with EtOAc. Wash the combined organics with saturated aqueous NaCl, dry over MgSO₄, filter and concentrate under vacuum. Triturate the material using hexanes in Et₂O and collect the resulting solid via filtration. Vacuum dry to give (S)—N-(8-bromo-5,6-dichloro-2,3-dihydro-1H-pyrrolo[1,2-a]indol-1-yl)acetamide (361 mg, 88%). ES/MS (m/z): 362.6/364.6 (M+H).

### Intermediate 565

### (S)—N-(5,6-Dichloro-8-hydroxy-2,3-dihydro-1H-pyrrolo[1,2-a]indol-1-yl)acetamide

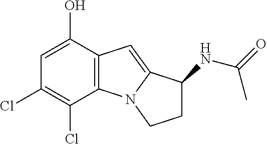 [SMILES: CC(=O)N[C@H]1CCn2c1cc1c(O)cc(Cl)c(Cl)c12]

Add NaOtBu (250 mg, 2.6 mmol) and water (2 mL) to (S)—N-(8-bromo-5,6-dichloro-2,3-dihydro-1H-pyrrolo[1,2-a]indol-1-yl)acetamide (360 mg, 1.0 mmol) and tBuBrettPhosG3 (90 mg, 0.1 mmol) in 1,4-dioxane (5 mL) with stirring and at ambient temperature. Heat the reaction to 65° C. for 3 hrs. Cool the reaction to ambient temperature, pour into saturated aqueous NH₄Cl and extract with EtOAc. Wash the combined organics with saturated aqueous NaCl, dry over MgSO₄, filter and concentrate under vacuum to give (S)—N-(5,6-dichloro-8-hydroxy-2,3-dihydro-1H-pyrrolo[1,2-a]indol-1-yl)acetamide (330 mg, used without further purification). ES/MS (m/z): (³⁵Cl/³⁷Cl) 298.8/300.8 (M+H).

### Intermediate 566

### (S)—N-(5,6-Dichloro-8-(cyanomethoxy)-2,3-dihydro-1H-pyrrolo[1,2-a]indol-1-yl)acetamide

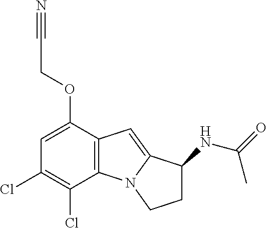 [SMILES: CC(=O)N[C@H]1CCn2c1cc1c(OCC#N)cc(Cl)c(Cl)c12]

Add bromoacetonitrile (105 μL, 1.5 mmol) to (S)—N-(5,6-dichloro-8-hydroxy-2,3-dihydro-1H-pyrrolo[1,2-a]indol-1-yl)acetamide (300 mg, 1.0 mmol) and K₂CO₃ (350 mg, 2.5 mmol) in DMF (6 mL) with stirring, under nitrogen and at ambient temperature. After 2 hr, pour the reaction into water and extract with EtOAc. Wash the combined organics with water and saturated aqueous NaCl. Dry the material over MgSO₄, filter and concentrate under vacuum. Triturate the residue using hexanes in Et₂O and collect the resulting solid via filtration. Vacuum dry to give (S)—N-(5,6-dichloro-8-(cyanomethoxy)-2,3-dihydro-1H-pyrrolo[1,2-a]indol-1-yl)acetamide (292 mg, 86%). ES/MS (m/z): (³⁵Cl/³⁷Cl) 337.8/339.8 (M+H).

### Intermediate 567

### (S)—N-(5,6-Dichloro-8-(cyanomethoxy)-9-iodo-2,3-dihydro-1H-pyrrolo[1,2-a]indol-1-yl)acetamide

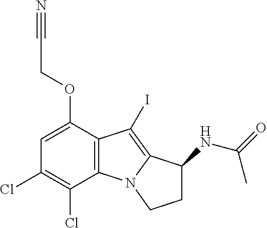 [SMILES: CC(=O)N[C@H]1CCn2c1c(I)c1c(OCC#N)cc(Cl)c(Cl)c12]

Add N-iodosuccinimide (220 mg, 0.96 mmol) to (S)—N-(5,6-dichloro-8-(cyanomethoxy)-2,3-dihydro-1H-pyrrolo[1,2-a]indol-1-yl)acetamide (292 mg, 0.86 mmol) in DMF (5 mL) with stirring, under nitrogen and at ambient temperature. After 2 hrs., pour the reaction into saturated aqueous Na₂S₂O₃ and extract with EtOAc. Wash the combined organics with water and saturated aqueous NaCl. Dry the material over MgSO₄, filter and concentrate under vacuum. Triturate the product from Et₂O/Hex and collect the resulting solid via filtration. Vacuum dry to give (S)—N-(5,6-dichloro-8-(cyanomethoxy)-9-iodo-2,3-dihydro-1H-pyrrolo[1,2-a]indol-1-yl)acetamide (262 mg, 65%). ES/MS (m/z): (³⁵Cl/³⁷Cl) 463.6/465.6 (M+H).

### Intermediate 568

### N-((1 S)-5, 6-Dichloro-8-(cyanomethoxy)-9-(1-(tetrahydro-2H-pyran-2-yl)-1H-pyrazol-4-yl)-2,3-dihydro-1H-pyrrolo[1,2-a]indol-1-yl)acetamide

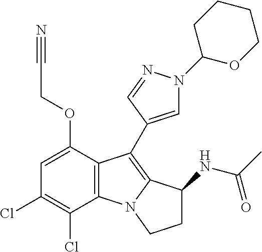 [SMILES: CC(=O)N[C@H]1CCn2c1c(-c1cnn(C3CCCCO3)c1)c1c(OCC#N)cc(Cl)c(Cl)c12]

Add K₂CO₃ (150 mg, 1.1 mmol), CH₃CN (5 mL) and water (1.3 mL) to tri-tert-butylphosphonium tetrafluoroborate (20 mg, 0.067 mmol) and crotylpalladium chloride dimer (10 mg, 0.025 mmol) with stirring, under N₂ and at ambient temperature. After 30 min, add the mixture to a microwave vial containing (S)—N-(5,6-dichloro-8-(cyanomethoxy)-9-iodo-2,3-dihydro-1H-pyrrolo[1,2-a]indol-1-yl)acetamide (250 mg, 0.540 mmol) and 1-(tetrahydro-2H-pyran-2-yl)-4-(4,4,5,5-tetramethyl-1,3,2-dioxaborolan-2-yl)-1H-pyrazole (310 mg, 0.22 mmol). Degas the reaction with a stream of N₂ while stirring. Cap the reaction and heat to 100° C. under microwave irradiation for 1 hr. Cool the reaction to ambient temperature, pour into water and extract with EtOAc. Wash the combined organics with saturated aqueous NaCl, dry over MgSO₄, filter and concentrate under vacuum. Purify via flash chromatography eluting with DCM in EtOAc and MeOH to give N-((1S)-5,6-dichloro-8-(cyanomethoxy)-9-(1-(tetrahydro-2H-pyran-2-yl)-1H-pyrazol-4-yl)-2,3-dihydro-1H-pyrrolo[1,2-a]indol-1-yl)acetamide (100 mg, 38%). ES/MS (m/z): (³⁵Cl/³⁷Cl) 487.8/489.8 (M+H).

### EXAMPLE 232

### (S)—N-(5,6-Dichloro-8-(cyanomethoxy)-9-(1H-pyrazol-4-yl)-2,3-dihydro-1H-pyrrolo[1,2-a]indol-1-yl)acetamide

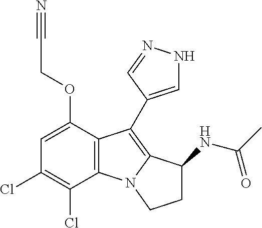 [SMILES: CC(=O)N[C@H]1CCn2c1c(-c1cn[nH]c1)c1c(OCC#N)cc(Cl)c(Cl)c12]

Add TFA (150 μL) to N-((1S)-5,6-dichloro-8-(cyanomethoxy)-9-(1-(tetrahydro-2H-pyran-2-yl)-1H-pyrazol-4-yl)-2,3-dihydro-1H-pyrrolo[1,2-a]indol-1-yl)acetamide (100 mg, 0.2 mmol) in DCM (2 mL) with stirring, under N₂ and at ambient temperature. Add additional TFA (150 μL) over 1 hr. intervals until the reaction is complete. Concentrate the reaction via a stream of N₂. Take up the residue in EtOAc and pour into saturated aqueous NaHCO₃. Separate the layers and extract the aqueous with EtOAc. Combine the organics, dry over MgSO₄, filter and concentrate under vacuum. Purify by reverse phase chromatography to give (S)—N-(5,6-dichloro-8-(cyanomethoxy)-9-(1H-pyrazol-4-yl)-2,3-dihydro-1H-pyrrolo[1,2-a]indol-1-yl)acetamide (57 mg, 69%). ES/MS (m/z): (³⁵Cl/³⁷Cl) 403.8/405.8 (M+H). ¹H NMR (DMSO-d6): 12.79 (br s, 1H), 8.46 (d, J=8.4 Hz, 1H), 7.74-7.72 (m, 2H), 6.98 (s, 1H), 5.42 (td, J=8.0, 3.2 Hz, 1H), 5.28 (s, 2H), 4.59-4.51 (m, 2H), 2.89-2.79 (m, 1H), 2.34-2.28 (m, 1H), 1.78 (s, 3H).

### Intermediate 569

### (S)—N-(5,6-Dichloro-8-ethoxy-2,3-dihydro-1H-pyrrolo[1,2-a]indol-1-yl)acetamide

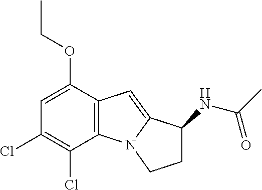 [SMILES: CCOc1cc(Cl)c(Cl)c2c1cc1n2CC[C@@H]1NC(C)=O]

Add iodoethane (70 μL, 0.87 mmol) to (S)—N-(5,6-dichloro-8-(cyanomethoxy)-9-(1H-pyrazol-4-yl)-2,3-dihydro-1H-pyrrolo[1,2-a]indol-1-yl)acetamide (168 mg, 0.56 mmol) and K₂CO₃ (195 mg, 1.41 mmol) in DMF (3 mL) with stirring, under N₂ and at ambient temperature. Add additional EtI and K₂CO₃ in 1 hr. intervals until the reaction is complete. Pour the reaction into water and extract with EtOAc. Wash the combined organics with water and saturated aqueous NaCl. Dry the material over MgSO₄, filter and concentrate under vacuum. Triturate the material from hexanes in Et₂O and the collect the resulting solid via filtration. Vacuum dry to give (S)—N-(5,6-dichloro-8-ethoxy-2,3-dihydro-1H-pyrrolo[1,2-a]indol-1-yl)acetamide (140 mg, 76%). ES/MS (m/z): (³⁵Cl/³⁷Cl) 326.8/328.8 (M+H).

### Intermediate 570

### (S)—N-(5,6-dichloro-8-ethoxy-9-iodo-2,3-dihydro-1H-pyrrolo[1,2-a]indol-1-yl)acetamide

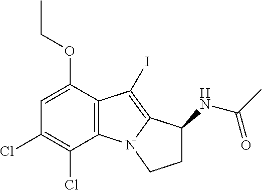 [SMILES: CCOc1cc(Cl)c(Cl)c2c1c(I)c1n2CC[C@@H]1NC(C)=O]

Prepare according to the procedure for (S)—N-(5,6-Dichloro-8-(cyanomethoxy)-9-iodo-2,3-dihydro-1H-pyrrolo[1,2-a]indol-1-yl)acetamide using (S)—N-(5,6-dichloro-8-ethoxy-2,3-dihydro-1H-pyrrolo[1,2-a]indol-1-yl)acetamide (142 mg, 0.427 mmol) to give (S)—N-(5,6-dichloro-8-ethoxy-9-iodo-2,3-dihydro-1H-pyrrolo[1,2-a]indol-1-yl)acetamide (132 mg, 68%). ES/MS (m/z): (³⁵Cl/³⁷Cl) 450.6/452.6 (M−H).

### Intermediate 571

### N-((1 S)-5, 6-Dichloro-8-ethoxy-9-(1-(tetrahydro-2H-pyran-2-yl)-1H-pyrazol-4-yl)-2,3-dihydro-1H-pyrrolo[1,2-a]indol-1-yl)acetamide

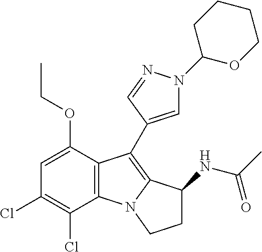 [SMILES: CCOc1cc(Cl)c(Cl)c2c1c(-c1cnn(C3CCCCO3)c1)c1n2CC[C@@H]1NC(C)=O]

Prepare according to the procedure for N-((1S)-5,6-dichloro-8-(cyanomethoxy)-9-(1-(tetrahydro-2H-pyran-2-yl)-1H-pyrazol-4-yl)-2,3-dihydro-1H-pyrrolo[1,2-a]indol-1-yl)acetamide using (S)—N-(5,6-dichloro-8-ethoxy-9-iodo-2,3-dihydro-1H-pyrrolo[1,2-a]indol-1-yl)acetamide (130 mg, 0.286 mmol) to give N-((1S)-5,6-dichloro-8-ethoxy-9-(1-(tetrahydro-2H-pyran-2-yl)-1H-pyrazol-4-yl)-2,3-dihydro-1H-pyrrolo[1,2-a]indol-1-yl)acetamide (76 mg, 55%). ES/MS (m/z): (³⁵Cl/³⁷Cl) 476.8/478.8 (M+H).

### EXAMPLE 233

### (S)—N-(5,6-Dichloro-8-ethoxy-9-(1H-pyrazol-4-yl)-2,3-dihydro-1H-pyrrolo[1,2-a]indol-1-yl)acetamide

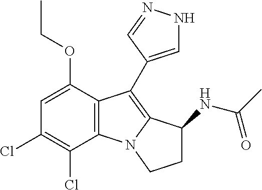 [SMILES: CCOc1cc(Cl)c(Cl)c2c1c(-c1cn[nH]c1)c1n2CC[C@@H]1NC(C)=O]

Add HCl (4.0M) in dioxane (400 μL, 1.6 mmol) to N-((1S)-5,6-dichloro-8-ethoxy-9-(1-(tetrahydro-2H-pyran-2-yl)-1H-pyrazol-4-yl)-2,3-dihydro-1H-pyrrolo[1,2-a]indol-1-yl)acetamide (75 mg, 0.157 mmol) in 1,4-dioxane (2 mL) and MeOH (600 μL) with stirring, under N₂ and at ambient temperature. Add additional HCl (400 μL) over 1 hr intervals until the reaction is complete. Carefully pour the reaction into saturated aqueous NaHCO₃ and extract with EtOAc (3×). Dry the combined organics over MgSO₄, filter and concentrate under vacuum. Triturate the material using Et₂O and collect the resulting solid via filtration. Dry under vacuum to give (S)—N-(5,6-dichloro-8-ethoxy-9-(1H-pyrazol-4-yl)-2,3-dihydro-1H-pyrrolo[1,2-a]indol-1-yl)acetamide (45 mg, 73%). ES/MS (m/z): (³⁵Cl/³⁷Cl) 392.8/394.8 (M+H). ¹H NMR (DMSO-d6): 12.67 (s, 1H), 8.45 (d, 1H), 7.74-7.60 (m, 2H), 6.72 (s, 1H), 5.41 (td, J=8.0, 3.1 Hz, 1H), 4.58-4.49 (m, 2H), 4.15-4.09 (m, 2H), 2.87-2.78 (m, 1H), 2.30-2.22 (m, 1H), 1.78 (s, 3H), 1.38-1.33 (t, J=7.0 Hz, 3H).

### Intermediate 572

### (S,E)-N-[3-[tert-Butyl(dimethyl) silyl] oxypropylidene]-2-methyl-propane-2-sulfinamide

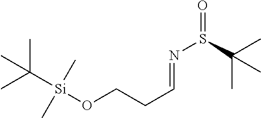 [SMILES: CC(C)(C)[S@](=O)/N=C/CCO[Si](C)(C)C(C)(C)C]

Prepare according to the procedure for (R,E)-N-[3-[tert-butyl(dimethyl) silyl] oxypropylidene]-2-methyl-propane-2-sulfinamide using (S)-(−)-2-methyl-2-propanesulfinamide (5 g, 25.75 mol) to give (S,E)-N-[3-[tert-butyl(dimethyl) silyl] oxypropylidene]-2-methyl-propane-2-sulfinamide (6.27 g, 79%). ¹H NMR (DMSO-d6): 7.96 (t, 1H), 3.94-3.90 (m, 2H), 2.73-2.66 (m, 2H), 1.11 (s, 9H), 0.85 (s, 9H), 0.04 (s, 6H).

### Intermediate 573

### (S)—N—((R)-1-(4-Bromo-6, 7-dichloro-1-(phenylsulfonyl)-1H-indol-2-yl)-3-((tert-butyl dimethyl silyl)oxy)propyl)-2-methylpropane-2-sulfinamide

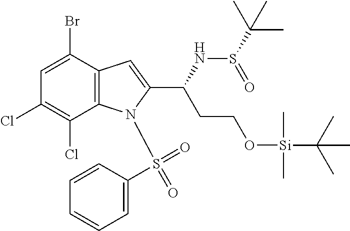 [SMILES: CC(C)(C)[S@](=O)N[C@H](CCO[Si](C)(C)C(C)(C)C)c1cc2c(Br)cc(Cl)c(Cl)c2n1S(=O)(=O)c1ccccc1]

Prepare according to the procedure for (R)—N-[(1S)-1-[1-(benzenesulfonyl)-4-bromo-6,7-dichloro-indol-2-yl]-3-[tert-butyl(dimethyl)silyl]oxy-propyl]-2-methyl-propane-2-sulfinamide using (S,E)-N-[3-[tert-butyl(dimethyl) silyl] oxypropylidene]-2-methyl-propane-2-sulfinamide (5 g, 12.3 mmol) to give (S)—N—((R)-1-(4-bromo-6,7-dichloro-1-(phenylsulfonyl)-1H-indol-2-yl)-3-((tert-butyldimethylsilyl)oxy)propyl)-2-methylpropane-2-sulfinamide (4.8 g, 56%). ES/MS (m/z): (³⁵Cl/³⁷Cl) 695.2/697.2 (M+H).

### Intermediate 574

### (S)—N—((R)-8-Bromo-5,6-dichloro-2,3-dihydro-1H-pyrrolo[1,2-a]indol-1-yl)-2-methylpropane-2-sulfinamide

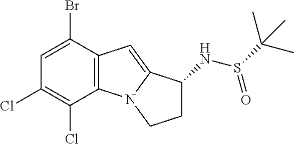 [SMILES: CC(C)(C)[S@](=O)N[C@@H]1CCn2c1cc1c(Br)cc(Cl)c(Cl)c12]

Prepare according to the procedure for (R)—N-[(3S)-5-bromo-7,8-dichloro-2,3-dihydro-1H-pyrrolo[1,2-a]indol-3-yl]-2-methyl-propane-2-sulfinamide using (S)—N—((R)-1-(4-bromo-6,7-dichloro-1-(phenylsulfonyl)-1H-indol-2-yl)-3-((tert-butyldimethylsilyl)oxy)propyl)-2-methylpropane-2-sulfinamide (4.8 g, 6.9 mmol) to give (S)—N—((R)-8-bromo-5,6-dichloro-2,3-dihydro-1H-pyrrolo[1,2-a]indol-1-yl)-2-methylpropane-2-sulfinamide (556 mg, 19%). ES/MS (m/z): (³⁵Cl/³⁷Cl) 422.6/424.6 (M−H).

### Intermediate 575

### (R)-8-Bromo-5, 6-dichloro-2,3-dihydro-1H-pyrrolo[1,2-a]indol-1-amine hydrochloride

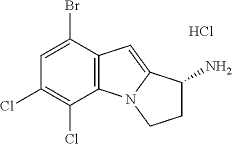 [SMILES: N[C@@H]1CCn2c1cc1c(Br)cc(Cl)c(Cl)c12 ; Cl]

Prepare according to the procedure for (S)-8-bromo-5,6-dichloro-2,3-dihydro-1H-pyrrolo[1,2-a]indol-1-amine hydrochloride using (S)—N4R)-8-bromo-5,6-dichloro-2,3-dihydro-1H-pyrrolo[1,2-a]indol-1-yl)-2-methylpropane-2-sulfinamide (555 mg, 1.31 mmol) to give (R)-8-bromo-5,6-dichloro-2,3-dihydro-1H-pyrrolo[1,2-a]indol-1-amine (446 mg, 96%). ES/MS (m/z): (³⁵Cl/³⁷Cl) 321.0/323.0 (M+H free base).

### Intermediate 576

### (R)—N-(8-Bromo-5,6-dichloro-2,3-dihydro-1H-pyrrolo[1,2-a]indol-1-yl)acetamide

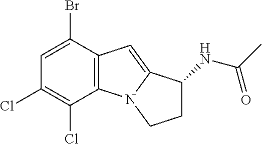 [SMILES: CC(=O)N[C@@H]1CCn2c1cc1c(Br)cc(Cl)c(Cl)c12]

Prepare according to the procedure for (S)—N-(8-bromo-5,6-dichloro-2,3-dihydro-1H-pyrrolo[1,2-a]indol-1-yl)acetamide using (R)-8-bromo-5,6-dichloro-2,3-dihydro-1H-pyrrolo[1,2-a]indol-1-amine (445 mg, 1.25 mmol) to give (R)—N-(8-bromo-5,6-dichloro-2,3-dihydro-1H-pyrrolo[1,2-a]indol-1-yl)acetamide (401 mg, 91%). ES/MS (m/z): (³⁵Cl/³⁷Cl) 362.7/364.6 (M+H).

### Intermediate 577

### (R)—N-(5,6-Dichloro-8-hydroxy-2,3-dihydro-1H-pyrrolo[1,2-a]indol-1-yl)acetamide

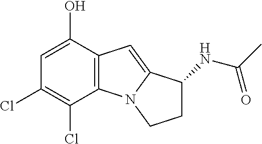 [SMILES: CC(=O)N[C@@H]1CCn2c1cc1c(O)cc(Cl)c(Cl)c12]

Prepare according to the procedure for (S)—N-(5,6-dichloro-8-hydroxy-2,3-dihydro-1H-pyrrolo[1,2-a]indol-1-yl)acetamide using (R)—N-(8-bromo-5,6-dichloro-2,3-dihydro-1H-pyrrolo[1,2-a]indol-1-yl)acetamide (410 mg, 1.13 mmol) to give (R)—N-(5,6-dichloro-8-hydroxy-2,3-dihydro-1H-pyrrolo[1,2-a]indol-1-yl)acetamide (351 mg, used without further purification). ES/MS (m/z): (³⁵Cl/³⁷Cl) 298.8/300.8 (M+H).

### Intermediate 578

### (R)—N-(5,6-Dichloro-8-(cyanomethoxy)-2,3-dihydro-1H-pyrrolo[1,2-a]indol-1-yl)acetamide

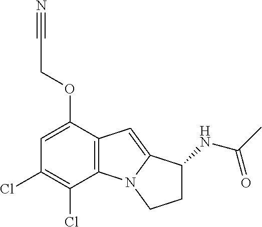 [SMILES: CC(=O)N[C@@H]1CCn2c1cc1c(OCC#N)cc(Cl)c(Cl)c12]

Prepare according to the procedure for (S)—N-(5,6-dichloro-8-(cyanomethoxy)-2,3-dihydro-1H-pyrrolo[1,2-a]indol-1-yl)acetamide using (R)—N-(5,6-dichloro-8-hydroxy-2,3-dihydro-1H-pyrrolo[1,2-a]indol-1-yl)acetamide (335 mg, 1.12 mmol) to give (R)—N-(5,6-dichloro-8-(cyanomethoxy)-2,3-dihydro-1H-pyrrolo[1,2-a]indol-1-yl)acetamide (274 mg, 72%). ES/MS (m/z): (³⁵Cl/³⁷Cl) 337.8/339.8 (M+H).

### Intermediate 579

### (R)—N-(5,6-Dichloro-8-(cyanomethoxy)-9-iodo-2,3-dihydro-1H-pyrrolo[1,2-a]indol-1-yl)acetamide

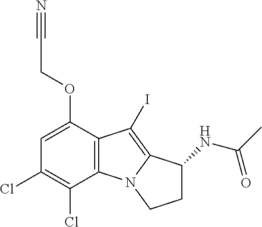 [SMILES: CC(=O)N[C@@H]1CCn2c1c(I)c1c(OCC#N)cc(Cl)c(Cl)c12]

Prepare according to the procedure for N-[(3S)-7,8-dichloro-5-(cyanomethoxy)-4-iodo-2,3-dihydro-1H-pyrrolo[1,2-a]indol-3-yl]acetamide using (R)—N-(5,6-dichloro-8-(cyanomethoxy)-2,3-dihydro-1H-pyrrolo[1,2-a]indol-1-yl)acetamide (270 mg, 0.8 mmol) to give (R)—N-(5,6-dichloro-8-(cyanomethoxy)-9-iodo-2,3-dihydro-1H-pyrrolo[1,2-a]indol-1-yl)acetamide (251 mg, 68%). ES/MS (m/z): (³⁵Cl/³⁷Cl) 463.6/465.6 (M+H).

### Intermediate 580

### N-((1R)-5, 6-Dichloro-8-(cyanomethoxy)-9-(1-(tetrahydro-2H-pyran-2-yl)-1H-pyrazol-4-yl)-2,3-dihydro-1H-pyrrolo[1,2-a]indol-1-yl)acetamide

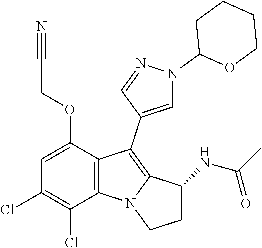 [SMILES: CC(=O)N[C@@H]1CCn2c1c(-c1cnn(C3CCCCO3)c1)c1c(OCC#N)cc(Cl)c(Cl)c12]

Prepare according to the procedure for N-((1S)-5,6-dichloro-8-(cyanomethoxy)-9-(1-(tetrahydro-2H-pyran-2-yl)-1H-pyrazol-4-yl)-2,3-dihydro-1H-pyrrolo[1,2-a]indol-1-yl)acetamide using (R)—N-(5,6-dichloro-8-(cyanomethoxy)-9-iodo-2,3-dihydro-1H-pyrrolo[1,2-a]indol-1-yl)acetamide (260 mg, 0.56 mmol) to give N-((1R)-5,6-dichloro-8-(cyanomethoxy)-9-(1-(tetrahydro-2H-pyran-2-yl)-1H-pyrazol-4-yl)-2,3-dihydro-1H-pyrrolo[1,2-a]indol-1-yl)acetamide (151 mg, 55%). ES/MS (m/z): (³⁵Cl/³⁷Cl) 487.8/489.8 (M+H).

### EXAMPLE 234

### (R)—N-(5,6-Dichloro-8-(cyanomethoxy)-9-(1H-pyrazol-4-yl)-2,3-dihydro-1H-pyrrolo[1,2-a]indol-1-yl)acetamide

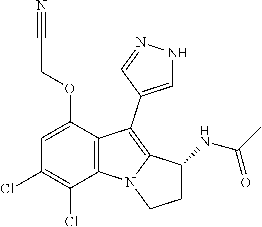 [SMILES: CC(=O)N[C@@H]1CCn2c1c(-c1cn[nH]c1)c1c(OCC#N)cc(Cl)c(Cl)c12]

Add TFA (250 μL) to N-((1R)-5,6-dichloro-8-(cyanomethoxy)-9-(1-(tetrahydro-2H-pyran-2-yl)-1H-pyrazol-4-yl)-2,3-dihydro-1H-pyrrolo[1,2-a]indol-1-yl)acetamide (151 mg, 0.31 mmol) in DCM (3 mL) with stirring, under N₂ and at ambient temperature. Add additional TFA (250 μL) over 1 hr., intervals until the reaction is complete. Concentrate the reaction via a stream of N₂ gas. Take up the residue in EtOAc and pour into saturated aqueous NaHCO₃. Separate the layers and extract the aqueous with EtOAc. Combine the organics, dry over MgSO₄, filter and concentrate under vacuum. Purify by reverse phase chromatography to give (R)—N-(5,6-dichloro-8-(cyanomethoxy)-9-(1H-pyrazol-4-yl)-2,3-dihydro-1H-pyrrolo[1,2-a]indol-1-yl)acetamide (71 mg, 57%). ES/MS (m/z): (³⁵Cl/³⁷Cl) 403.8/405.8 (M+H). ¹H NMR (DMSO-d6): 12.79 (br s, 1H), 8.46 (d, J=8.4 Hz, 1H), 7.74-7.72 (m, 2H), 6.98 (s, 1H), 5.42 (td, J=8.0, 3.2 Hz, 1H), 5.28 (s, 2H), 4.59-4.51 (m, 2H), 2.89-2.79 (m, 1H), 2.34-2.28 (m, 1H), 1.78 (s, 3H).

### Intermediate 581

### (R)—N-(5,6-Dichloro-8-ethoxy-2,3-dihydro-1H-pyrrolo[1,2-a]indol-1-yl)acetamide

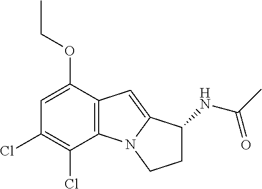 [SMILES: CCOc1cc(Cl)c(Cl)c2c1cc1n2CC[C@H]1NC(C)=O]

Prepare according to procedure for (S)—N-(5,6-dichloro-8-ethoxy-2,3-dihydro-1H-pyrrolo[1,2-a]indol-1-yl)acetamide using (R)—N-(5,6-dichloro-8-hydroxy-2,3-dihydro-1H-pyrrolo[1,2-a]indol-1-yl)acetamide (180 mg, 0.6 mmol) to give (R)—N-(5,6-dichloro-8-ethoxy-2,3-dihydro-1H-pyrrolo[1,2-a]indol-1-yl)acetamide (148 mg, 75%). ES/MS (m/z): (³⁵Cl/³⁷Cl) 326.8/328.8 (M+H).

### Intermediate 582

### (R)—N-(5,6-Dichloro-8-ethoxy-9-iodo-2,3-dihydro-1H-pyrrolo[1,2-a]indol-1-yl)acetamide

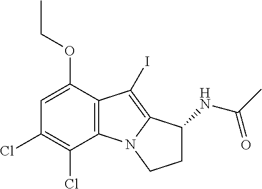 [SMILES: CCOc1cc(Cl)c(Cl)c2c1c(I)c1n2CC[C@H]1NC(C)=O]

Prepare according to the procedure for (S)—N-(5,6-dichloro-8-ethoxy-9-iodo-2,3-dihydro-1H-pyrrolo[1,2-a]indol-1-yl)acetamide using (R)—N-(5,6-dichloro-8-ethoxy-2,3-dihydro-1H-pyrrolo[1,2-a]indol-1-yl)acetamide (145 mg, 0.443 mmol) to give (R)—N-(5,6-dichloro-8-ethoxy-9-iodo-2,3-dihydro-1H-pyrrolo[1,2-a]indol-1-yl)acetamide (136 mg, 68%). ES/MS (m/z): (³⁵Cl/³⁷Cl) 453.0/455.0 (M+H).

### Intermediate 583

### N-((1R)-5,6-Dichloro-8-ethoxy-9-(1-(tetrahydro-2H-pyran-2-yl)-1H-pyrazol-4-yl)-2,3-dihydro-1H-pyrrolo[1,2-a]indol-1-yl)acetamide

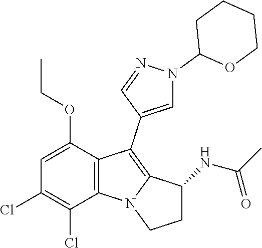 [SMILES: CCOc1cc(Cl)c(Cl)c2c1c(-c1cnn(C3CCCCO3)c1)c1n2CC[C@H]1NC(C)=O]

Prepare according to the procedure for (S)—N-(5,6-dichloro-8-ethoxy-9-(1H-pyrazol-4-yl)-2,3-dihydro-1H-pyrrolo[1,2-a]indol-1-yl)acetamide using (R)—N-(5,6-dichloro-8-ethoxy-9-iodo-2,3-dihydro-1H-pyrrolo[1,2-a]indol-1-yl)acetamide (100 mg, 0.22 mmol) to give N-((1R)-5,6-dichloro-8-ethoxy-9-(1-(tetrahydro-2H-pyran-2-yl)-1H-pyrazol-4-yl)-2,3-dihydro-1H-pyrrolo[1,2-a]indol-1-yl)acetamide (73 mg, 69%). ES/MS (m/z): (³⁵Cl/³⁷Cl) 476.8/479.0 (M+H).

### EXAMPLE 235

### (R)—N-(5,6-Dichloro-8-ethoxy-9-(1H-pyrazol-4-yl)-2,3-dihydro-1H-pyrrolo[1,2-a]indol-1-yl)acetamide

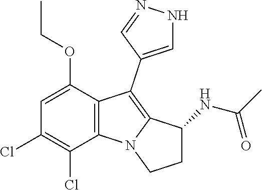 [SMILES: CCOc1cc(Cl)c(Cl)c2c1c(-c1cn[nH]c1)c1n2CC[C@H]1NC(C)=O]

Prepare according to the procedure for (S)—N-(5,6-dichloro-8-ethoxy-9-(1H-pyrazol-4-yl)-2,3-dihydro-1H-pyrrolo[1,2-a]indol-1-yl)acetamide using N-((1R)-5,6-dichloro-8-ethoxy-9-(1-(tetrahydro-2H-pyran-2-yl)-1H-pyrazol-4-yl)-2,3-dihydro-1H-pyrrolo[1,2-a]indol-1-yl)acetamide (70 mg, 0.146 mmol) to give (R)—N-(5,6-dichloro-8-ethoxy-9-(1H-pyrazol-4-yl)-2,3-dihydro-1H-pyrrolo[1,2-a]indol-1-yl)acetamide (39 mg, 68%). ES/MS (m/z): (³⁵Cl/³⁷Cl) 392.8/394.8 (M+H). ¹H NMR (DMSO-d6): 12.67 (s, 1H), 8.45 (d, 1H), 7.74-7.60 (m, 2H), 6.72 (s, 1H), 5.41 (td, J=8.0, 3.1 Hz, 1H), 4.58-4.49 (m, 2H), 4.15-4.09 (m, 2H), 2.87-2.78 (m, 1H), 2.30-2.22 (m, 1H), 1.78 (s, 3H), 1.38-1.33 (t, J=7.0 Hz, 3H).

### Intermediate 584

### 2-Acetamido-N-(5,6-dichloro-9-(1-(tetrahydro-2H-pyran-2-yl)-1H-pyrazol-4-yl)-2,3-dihydro-1H-pyrrolo[1,2-a]indol-1-yl)acetamide

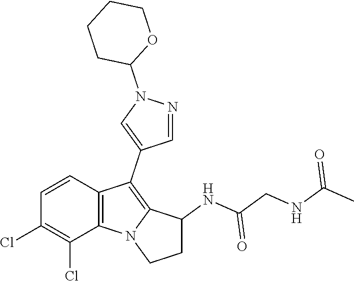 [SMILES: CC(=O)NCC(=O)NC1CCn2c1c(-c1cnn(C3CCCCO3)c1)c1ccc(Cl)c(Cl)c12]

Add HATU (150 mg, 0.386 mmol) and TEA (200 μL, 1.43 mmol) to 5,6-dichloro-9-(1-(tetrahydro-2H-pyran-2-yl)-1H-pyrazol-4-yl)-2,3-dihydro-1H-pyrrolo[1,2-a]indol-1-amine (120 mg, 0.31 mmol) and N-acetylglycine (45 mg, 0.38 mmol) in DMF (4 mL) with stirring, under N₂ and at ambient temperature. After 3 hrs., pour the reaction into water and extract with EtOAc. Wash the combined organics with water and saturated aqueous NaCl. Dry the material over MgSO₄, filter and concentrate under vacuum to give 2-acetamido-N-(5,6-dichloro-9-(1-(tetrahydro-2H-pyran-2-yl)-1H-pyrazol-4-yl)-2,3-dihydro-1H-pyrrolo[1,2-a]indol-1-yl)acetamide (245 mg, crude). ES/MS (m/z): (³⁵Cl/³⁷Cl) 489.8/491.8 (M+H).

### EXAMPLE 236

### 2-Acetamido-N-(5,6-dichloro-9-(1H-pyrazol-4-yl)-2,3-dihydro-1H-pyrrolo[1,2-a]indol-1-yl)acetamide

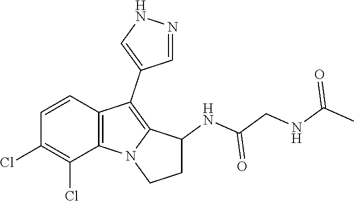 [SMILES: CC(=O)NCC(=O)NC1CCn2c1c(-c1cn[nH]c1)c1ccc(Cl)c(Cl)c12]

Add HCl (4.0M) in dioxane (1 mL) to 2-acetamido-N-(5,6-dichloro-9-(1-(tetrahydro-2H-pyran-2-yl)-1H-pyrazol-4-yl)-2,3-dihydro-1H-pyrrolo[1,2-a]indol-1-yl)acetamide (220 mg, 0.435 mmol) in 1,4-dioxane (3 mL) and MeOH (1 mL) with stirring, under N₂ and at ambient temperature. After 1 hr., concentrate the reaction under vacuum. Purify by reverse phase chromatography to give 2-acetamido-N-(5,6-dichloro-9-(1H-pyrazol-4-yl)-2,3-dihydro-1H-pyrrolo[1,2-a]indol-1-yl)acetamide (43 mg, 24%). ES/MS (m/z): (³⁵Cl/³⁷Cl) 406.0/408.0 (M+H). ¹H NMR (DMSO-d6): 12.97-12.88 (m, 1H), 8.58 (d, J=8.5 Hz, 1H), 8.11 (t, J=5.8 Hz, 1H), 7.85 (b s, 2H), 7.77 (d, J=8.6 Hz, 1H), 7.23 (d, J=8.6 Hz, 1H), 5.54 (td, J=8.2, 2.8 Hz, 1H), 4.56 (dd, J=5.7, 8.0 Hz, 2H), 3.64 (d, J=5.8 Hz, 2H), 2.97-2.88 (m, 1H), 2.34-2.29 (m, 1H), 1.84 (s, 3H).

### Intermediate 585

### N-(5, 6-Dichloro-9-(1-(tetrahydro-2H-pyran-2-yl)-1H-pyrazol-4-yl)-2,3-dihydro-1H-pyrrolo[1,2-a]indol-1-yl)-2-hydroxyacetamide

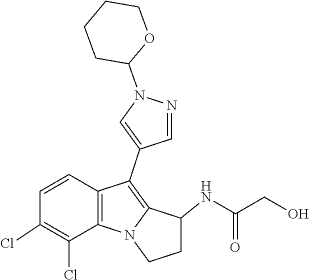 [SMILES: O=C(CO)NC1CCn2c1c(-c1cnn(C3CCCCO3)c1)c1ccc(Cl)c(Cl)c12]

Prepare according to the procedure for 2-acetamido-N-(5,6-dichloro-9-(1-(tetrahydro-2H-pyran-2-yl)-1H-pyrazol-4-yl)-2,3-dihydro-1H-pyrrolo[1,2-a]indol-1-yl)acetamide using 5,6-dichloro-9-(1-(tetrahydro-2H-pyran-2-yl)-1H-pyrazol-4-yl)-2,3-dihydro-1H-pyrrolo[1,2-a]indol-1-amine (130 mg, 0.33 mmol) and glycolic acid (30 mg, 0.39 mmol) to give N-(5,6-dichloro-9-(1-(tetrahydro-2H-pyran-2-yl)-1H-pyrazol-4-yl)-2,3-dihydro-1H-pyrrolo[1,2-a]indol-1-yl)-2-hydroxyacetamide (260 mg, used without further purification). ES/MS (m/z): (³⁵Cl/³⁷Cl) 448.8/450.8 (M+H).

### EXAMPLE 237

### N-(5,6-Dichloro-9-(1H-pyrazol-4-yl)-2,3-dihydro-1H-pyrrolo[1,2-a]indol-1-yl)-2-hydroxyacetamide

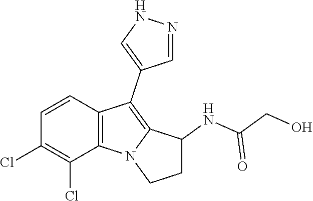 [SMILES: O=C(CO)NC1CCn2c1c(-c1cn[nH]c1)c1ccc(Cl)c(Cl)c12]

Add HCl (4.0M) in dioxane (1 mL) to N-(5,6-dichloro-9-(1-(tetrahydro-2H-pyran-2-yl)-1H-pyrazol-4-yl)-2,3-dihydro-1H-pyrrolo[1,2-a]indol-1-yl)-2-hydroxyacetamide (200 mg, 0.43 mmol) in 1,4-dioxane (3 mL) and MeOH (1 mL) with stirring, under nitrogen and at ambient temperature. After 1 hr., concentrate the reaction under reduced pressure. Take up the residue in EtOAc and pour into saturated aqueous NaHCO₃. Separate the layers and extract the organics with EtOAc. Dry the organic phase over MgSO₄, filter and concentrate under vacuum. Purify via reverse phase chromatography to give N-(5,6-dichloro-9-(1H-pyrazol-4-yl)-2,3-dihydro-1H-pyrrolo[1,2-a]indol-1-yl)-2-hydroxyacetamide (24 mg, 15%). ES/MS (m/z): (³⁵Cl/³⁷Cl) 364.8/366.8 (M+H). ¹H NMR (DMSO-d6): 12.92 (s, 1H), 8.45 (d, J=9.0 Hz, 1H), 7.97 (bs, 1H), 7.78 (d, 1H), 7.74 (bs, 1H), 7.22 (d, 8.6 Hz, 1H), 5.66 (td, J=8.6, 3.2 Hz, 1H), 5.45 (t, J=5.8 Hz, 1H), 4.66-4.53 (m, 2H), 3.82 (t, J=5.7 Hz, 2H), 3.00-2.90 (m, 1H), 2.40-2.30 (m, 1H).

### Intermediate 586

### N-(5, 6-Dichloro-9-(1-(tetrahydro-2H-pyran-2-yl)-1H-pyrazol-4-yl)-2,3-dihydro-1H-pyrrolo[1,2-a]indol-1-yl)methanesulfonamide

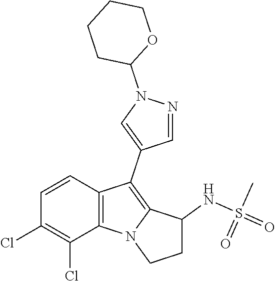 [SMILES: CS(=O)(=O)NC1CCn2c1c(-c1cnn(C3CCCCO3)c1)c1ccc(Cl)c(Cl)c12]

Add methanesulfonyl chloride (30 μL, 0.39 mmol) to 5,6-dichloro-9-(1-(tetrahydro-2H-pyran-2-yl)-1H-pyrazol-4-yl)-2,3-dihydro-1H-pyrrolo[1,2-a]indol-1-amine (115 mg, 0.29 mmol) and TEA (80 μL, 0.57 mmol) in DCM (3 mL) with stirring, under N₂ and at 0° C. Gradually warm the reaction to ambient temperature over 1 hr. Pour the reaction into water and extract with DCM. Wash the combined organics with saturated aqueous NaCl, dry over MgSO₄, filter and concentrate under vacuum to give N-(5,6-dichloro-9-(1-(tetrahydro-2H-pyran-2-yl)-1H-pyrazol-4-yl)-2,3-dihydro-1H-pyrrolo[1,2-a]indol-1-yl)methanesulfonamide (141 mg, 99%). ES/MS (m/z): (³⁵Cl/³⁷Cl) 468.8/470.8 (M+H).

### EXAMPLE 238

### N-(5,6-Dichloro-9-(1H-pyrazol-4-yl)-2,3-dihydro-1H-pyrrolo[1,2-a]indol-1-yl)methanesulfonamide

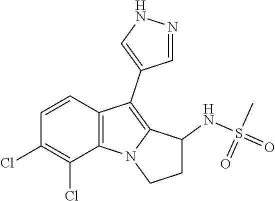 [SMILES: CS(=O)(=O)NC1CCn2c1c(-c1cn[nH]c1)c1ccc(Cl)c(Cl)c12]

Add HCl (4.0 M) in dioxane (1 mL) to N-(5,6-dichloro-9-(1-(tetrahydro-2H-pyran-2-yl)-1H-pyrazol-4-yl)-2,3-dihydro-1H-pyrrolo[1,2-a]indol-1-yl)methanesulfonamide (140 mg, 0.29 mmol) in 1,4-dioxane (3 mL) and MeOH (1 mL) with stirring, under N₂ and at ambient temperature. After 1 hr., concentrate the reaction under vacuum. Take up the residue in EtOAc and pour into saturated aqueous NaHCO₃. Separate the layers and extract the aqueous with EtOAc. Dry the organics over MgSO₄, filter and concentrate under vacuum. Purify the material via reverse phase chromatography to give N-(5,6-dichloro-9-(1H-pyrazol-4-yl)-2,3-dihydro-1H-pyrrolo[1,2-a]indol-1-yl)methanesulfonamide (24 mg, 21%). ES/MS (m/z): (³⁵Cl/³⁷Cl) 384.8/386.8 (M+H). ¹H NMR (DMSO-d6): 13.00-12.97 (m, 1H), 8.15 (s, 1H), 7.98-7.96 (m, 1H), 7.80-7.77 (m, 2H), 7.24 (d, J=8.6 Hz, 1H), 5.22-5.17 (m, 1H), 4.62-4.54 (m, 2H), 3.03 (m, 1H), 2.98 (s, 3H), 2.55 (m, 1H).

### Intermediate 587

### N-(5,6-Dichloro-9-(1-(tetrahydro-2H-pyran-2-yl)-1H-pyrazol-4-yl)-2,3-dihydro-1H-pyrrolo[1,2-a]indol-1-yl)propionamide

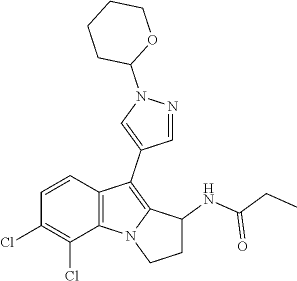 [SMILES: CCC(=O)NC1CCn2c1c(-c1cnn(C3CCCCO3)c1)c1ccc(Cl)c(Cl)c12]

Prepare according to the procedure for 2-acetamido-N-(5,6-dichloro-9-(1-(tetrahydro-2H-pyran-2-yl)-1H-pyrazol-4-yl)-2,3-dihydro-1H-pyrrolo[1,2-a]indol-1-yl)acetamide using 5,6-dichloro-9-(1-(tetrahydro-2H-pyran-2-yl)-1H-pyrazol-4-yl)-2,3-dihydro-1H-pyrrolo[1,2-a]indol-1-amine (55 mg, 0.14 mmol) and propionyl chloride (15 μL, 0.17 mmol) to give N-(5,6-dichloro-9-(1-(tetrahydro-2H-pyran-2-yl)-1H-pyrazol-4-yl)-2,3-dihydro-1H-pyrrolo[1,2-a]indol-1-yl)propionamide (27 mg, 42%). ES+MS (m/z): (³⁵Cl/³⁷Cl) 447.4/449.3 (M+H).

### EXAMPLE 239

### N-(5,6-Dichloro-9-(1H-pyrazol-4-yl)-2,3-dihydro-1H-pyrrolo[1,2-a]indol-1-yl)propionamide

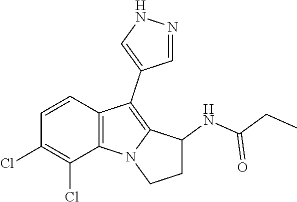 [SMILES: CCC(=O)NC1CCn2c1c(-c1cn[nH]c1)c1ccc(Cl)c(Cl)c12]

Add HCl (4.0 M) in dioxane (150 μL) to N-(5,6-dichloro-9-(1-(tetrahydro-2H-pyran-2-yl)-1H-pyrazol-4-yl)-2,3-dihydro-1H-pyrrolo[1,2-a]indol-1-yl)propionamide (27 mg 0.058 mmol) in 1,4-dioxane (2 mL) and MeOH (0.75 mL) with stirring, under N₂ and at ambient temperature. After 1 hr., concentrate the reaction under vacuum. Take up the residue in EtOAc and wash with saturated aqueous NaHCO₃. Dry the organics over MgSO₄, filter and concentrate under vacuum to give N-(5,6-dichloro-9-(1H-pyrazol-4-yl)-2,3-dihydro-1H-pyrrolo[1,2-a]indol-1-yl)propionamide (21 mg, 96%). ES/MS (m/z): (³⁵Cl/³⁷Cl) 362.8/364.8 (M+H). ¹H NMR (DMSO-d6): 12.96-12.89 (m, 1H), 8.44 (d, J=8.7 Hz, 1H), 7.95 (s, 1H), 7.79-7.72 (m, 2H), 7.23 (d, J=8.6 Hz, 1H), 5.60 (td, J=8.3, 3.5 Hz, 1H), 4.58-4.53 (m, 2H), 2.97-2.88 (m, 1H), 2.34-2.30 (m, 1H), 2.08-2.02 (m, 2H), 0.98 (t, J=7.6 Hz, 3H).

### Intermediate 588

### N-(5,6-Dichloro-9-(1-(tetrahydro-2H-pyran-2-yl)-1H-pyrazol-4-yl)-2,3-dihydro-1H-pyrrolo[1,2-a]indol-1-yl)-3-methoxypropanamide

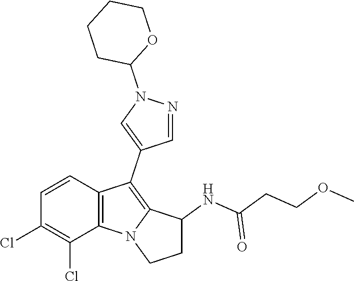 [SMILES: COCCC(=O)NC1CCn2c1c(-c1cnn(C3CCCCO3)c1)c1ccc(Cl)c(Cl)c12]

Prepare according to the procedure for 2-acetamido-N-(5,6-dichloro-9-(1-(tetrahydro-2H-pyran-2-yl)-1H-pyrazol-4-yl)-2,3-dihydro-1H-pyrrolo[1,2-a]indol-1-yl)acetamide using 5,6-dichloro-9-(1-(tetrahydro-2H-pyran-2-yl)-1H-pyrazol-4-yl)-2,3-dihydro-1H-pyrrolo[1,2-a]indol-1-amine (105 mg, 0.268 mmol) and 3-methoxypropanoyl chloride (35 μL, 0.295 mmol) to give N-(5,6-dichloro-9-(1-(tetrahydro-2H-pyran-2-yl)-1H-pyrazol-4-yl)-2,3-dihydro-1H-pyrrolo[1,2-a]indol-1-yl)-3-methoxypropanamide (121 mg, 92%). ES/MS (m/z): (³⁵Cl/³⁷Cl) 477.2/479.2 (M+H).

### EXAMPLE 240

### N-(5,6-Dichloro-9-(1H-pyrazol-4-yl)-2,3-dihydro-1H-pyrrolo[1,2-a]indol-1-yl)-3-methoxypropanamide

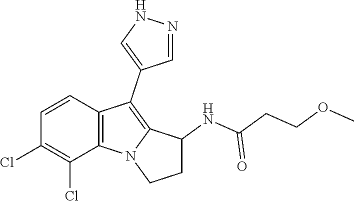 [SMILES: COCCC(=O)NC1CCn2c1c(-c1cn[nH]c1)c1ccc(Cl)c(Cl)c12]

Add HCl (4.0M) in 1,4-dioxane (600 μL) to N-(5,6-dichloro-9-(1-(tetrahydro-2H-pyran-2-yl)-1H-pyrazol-4-yl)-2,3-dihydro-1H-pyrrolo[1,2-a]indol-1-yl)-3-methoxypropanamide (121 mg, 0.245 mmol) in 1,4-dioxane (3 mL) and MeOH (1 mL) with stirring, under N₂ and at ambient temperature. After 1 hr., concentrate the reaction under vacuum. Take up the material in EtOAc and pour into saturated aqueous NaHCO₃. Separate the layers and extract the aqueous with EtOAc. Dry the combined organics over MgSO₄, filter and concentrate under vacuum. Purify the material via reverse phase chromatography to give N-(5,6-dichloro-9-(1H-pyrazol-4-yl)-2,3-dihydro-1H-pyrrolo[1,2-a]indol-1-yl)-3-methoxypropanamide (37 mg, 37%). ES/MS (m/z): (³⁵Cl/³⁷Cl) 390.8/392.8 (M−H). ¹H NMR (DMSO-d6): 12.96-12.93 (m, 1H), 8.58 (d, J=8.6 Hz, 1H), 7.95 (s, 1H), 7.79-7.75 (m, 2H), 7.23 (d, J=8.6 Hz, 1H), 5.54 (td, J=8.2, 2.9 Hz, 1H), 4.59-4.50 (m, 2H), 3.55-3.50 (m, 2H), 3.20 (s, 3H), 3.02-2.88 (m, 1H), 2.33-2.27 (m, 3H).

### EXAMPLE 241

### 5,6-Dichloro-9-(1H-pyrazol-4-yl)-2,3-dihydro-1H-pyrrolo[1,2-a]indol-1-amine

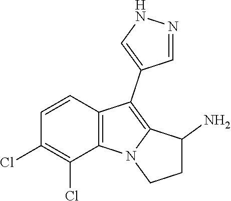 [SMILES: NC1CCn2c1c(-c1cn[nH]c1)c1ccc(Cl)c(Cl)c12]

Add HCl (4.0 M) in 1,4-dioxane (100 μL) to 5,6-dichloro-9-(1-(tetrahydro-2H-pyran-2-yl)-1H-pyrazol-4-yl)-2,3-dihydro-1H-pyrrolo[1,2-a]indol-1-amine (115 mg, 0.285 mmol) in 1,4-dioxane (3 mL) and MeOH (1 mL) with stirring, under nitrogen and at ambient temperature. After 1 hr, concentrate the reaction under reduced pressure. Take up the residue in EtOAc and wash with saturated aqueous NaHCO₃. Dry the organics over MgSO₄, filter and concentrate under vacuum. Purify the material via reverse phase chromatography to give 5,6-dichloro-9-(1H-pyrazol-4-yl)-2,3-dihydro-1H-pyrrolo[1,2-a]indol-1-amine (28 mg, 31%). ES/MS (m/z): (³⁵Cl/³⁷Cl) 289.8/291.8 (M+H). ¹H NMR (DMSO-d6): 13.03-13.01 (m, 1H), 8.28-8.22 (m, 2H), 7.71 (d, J=8.6 Hz, 1H), 7.19 (d, J=8.6 Hz, 1H), 4.60-4.52 (m, 3H), 2.85-2.79 (m, 1H), 2.34-2.28 (m, 3H).

### Intermediate 589

### Ethyl (E)-2-(5,6-dichloro-9-(1-(tetrahydro-2H-pyran-2-yl)-1H-pyrazol-4-yl)-2,3-dihydro-1H-pyrrolo[1,2-a]indol-1-ylidene)acetate

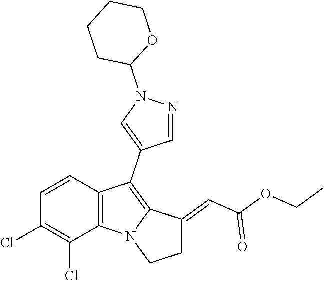 [SMILES: CCOC(=O)/C=C1\CCn2c1c(-c1cnn(C3CCCCO3)c1)c1ccc(Cl)c(Cl)c12]

Add (carbethoxymethylene)triphenylphosphorane (750 mg, 2.15 mmol) to 5,6-dichloro-9-(1-(tetrahydro-2H-pyran-2-yl)-1H-pyrazol-4-yl)-2,3-dihydro-1H-pyrrolo[1,2-a]indol-1-one (425 mg, 1.06 mmol) in 1,4-dioxane (5 mL) in a microwave vial with stirring, under N₂ and at ambient temperature. Heat the reaction to 170° C. under microwave irradiation for 90 min. Cool the reaction to ambient temperature, pour into water and extract with EtOAc. Dry the combined organics over MgSO₄, filter and concentrate under vacuum. Purify the material via flash chromatography eluting with EtOAc in DCM to give ethyl (E)-2-(5,6-dichloro-9-(1-(tetrahydro-2H-pyran-2-yl)-1H-pyrazol-4-yl)-2,3-dihydro-1H-pyrrolo[1,2-a]indol-1-ylidene)acetate (135 mg, 27%). ES-MS: (m/z): (³⁵Cl/³⁷Cl) 459.8/461.8 (M+H).

### Intermediate 590

### Ethyl 2-(5,6-dichloro-9-(1-(tetrahydro-2H-pyran-2-yl)-1H-pyrazol-4-yl)-2,3-dihydro-1H-pyrrolo[1,2-a]indol-1-yl)acetate

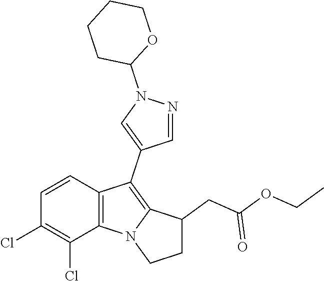 [SMILES: CCOC(=O)CC1CCn2c1c(-c1cnn(C3CCCCO3)c1)c1ccc(Cl)c(Cl)c12]

Add EtOAc (3.5 mL) and 5% Pt/C to under N₂. Add ethyl (E)-2-(5,6-dichloro-9-(1-(tetrahydro-2H-pyran-2-yl)-1H-pyrazol-4-yl)-2,3-dihydro-1H-pyrrolo[1,2-a]indol-1-ylidene)acetate (135 mg, 0.28 mmol) in EtOAc (3.5 mL). Purge with N₂ gas and then pressurize to 60 psi with H₂ gas. Shake the reaction under pressure for 16 hrs. Carefully filter the reaction over diatomaceous earth and rinse with EtOAc. Concentrate the filtrate under vacuum to give ethyl 2-(5,6-dichloro-9-(1-(tetrahydro-2H-pyran-2-yl)-1H-pyrazol-4-yl)-2,3-dihydro-1H-pyrrolo[1,2-a]indol-1-yl)acetate (136 mg, 99+%). ES/MS (m/z): (³⁵Cl/³⁷Cl) 461.8/463.8 (M+H).

### Intermediate 591

### 2-(5,6-Dichloro-9-(1-(tetrahydro-2H-pyran-2-yl)-1H-pyrazol-4-yl)-2,3-dihydro-1H-pyrrolo[1,2-a]indol-1-yl)ethan-1-01

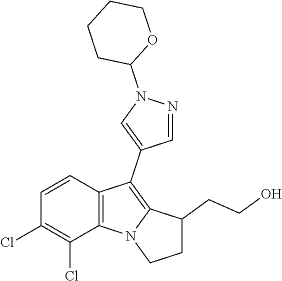 [SMILES: OCCC1CCn2c1c(-c1cnn(C3CCCCO3)c1)c1ccc(Cl)c(Cl)c12]

Add LAH (2.0M in THF, 170 μL, 0.34 mmol) dropwise to ethyl 2-(5,6-dichloro-9-(1-(tetrahydro-2H-pyran-2-yl)-1H-pyrazol-4-yl)-2,3-dihydro-1H-pyrrolo[1,2-a]indol-1-yl)acetate (136 mg 0.28 mmol) in THF (3 mL) with stirring under N₂ and at −78° C. Allow the reaction to warm to 0° C. After 30 min at 0° C., carefully quench the reaction with 15 of water, 15 μL of 15% NaOH and 45 μL of water successively. Dilute the solution with Et₂O and stir at ambient temperature for 30 min. Add MgSO₄ to the stirred solution, filter over diatomaceous earth and rinse with EtOAc. Concentrate the filtrate under vacuum to give 2-(5,6-dichloro-9-(1-(tetrahydro-2H-pyran-2-yl)-1H-pyrazol-4-yl)-2,3-dihydro-1H-pyrrolo[1,2-a]indol-1-yl)ethan-1-ol (130 mg, used without further purification). ES/MS (m/z): (³⁵Cl/³⁷Cl) 420.2/422.2 (M+H).

### Intermediate 592

### 2-(5, 6-Dichloro-9-(1H-pyrazol-4-yl)-2,3-dihydro-1H-pyrrolo[1,2-a]indol -1-yl)ethan-1-ol

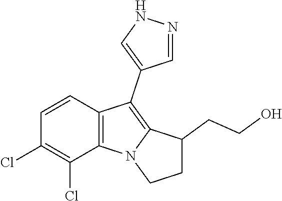 [SMILES: OCCC1CCn2c1c(-c1cn[nH]c1)c1ccc(Cl)c(Cl)c12]

Add HCl (4.0M) in 1,4-dioxane (750 μL) to 2-(5,6-dichloro-9-(1-(tetrahydro-2H-pyran-2-yl)-1H-pyrazol-4-yl)-2,3-dihydro-1H-pyrrolo[1,2-a]indol-1-yl)ethan-1-ol (130 mg, 0.3 mmol) in 1,4-dioxane (3 mL) and MeOH (1 mL) with stirring, under N₂ at ambient temperature. Add additional HCl (4.0M) in dioxane (150 μL) over 1 hr intervals until the reaction is complete. Concentrate the reaction under vacuum. Take up the residue in EtOAc and wash with saturated aqueous NaHCO₃. Dry the organics over MgSO₄, filter and concentrate under vacuum. Purify the material via reverse phase chromatography to give 2-(5,6-dichloro-9-(1H-pyrazol-4-yl)-2,3-dihydro-1H-pyrrolo[1,2-a]indol-1-yl)ethan-1-ol (64 mg, 62%). ES/MS (m/z): (³⁵Cl/³⁷Cl) 336.0/338.0 (M+H). 1H NMR (DMSO-d6): 12.99-12.97 (m, 1H), 8.05-8.02 (m, 1H), 7.82-7.78 (m, 1H), 7.66-7.64 (m, 1H), 7.19-7.17 (m, 1H), 4.64-4.61 (m, 1H), 4.55-4.51 (m, 2H), 3.68-3.65 (m, 1H), 3.53-3.49 (m, 2H), 2.78-2.76 (m, 1H), 2.39-2.36 (m, 1H), 1.94-1.91 (m, 1H), 1.58-1.54 (m, 1H).

### EXAMPLES 242 & 243

### 2-(5, 6-Dichloro-9-(1H-pyrazol-4-yl)-2,3-dihydro-1H-pyrrolo[1,2-a]indol-1-yl)ethan-1-ol isomer 1 and isomer 2

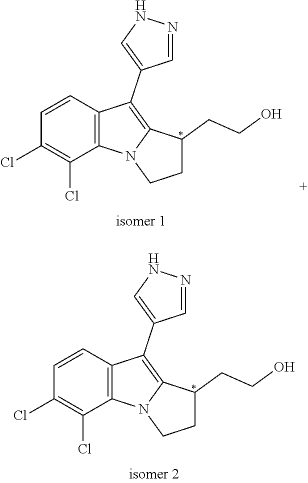 [SMILES: OCCC1CCn2c1c(-c1cn[nH]c1)c1ccc(Cl)c(Cl)c12 ; OCCC1CCn2c1c(-c1cn[nH]c1)c1ccc(Cl)c(Cl)c12]

Purify 2-(5,6-dichloro-9-(1H-pyrazol-4-yl)-2,3-dihydro-1H-pyrrolo[1,2-a]indol-1-yl)ethan-1-ol (64 mg, 0.185 mmol) by SFC to give the titled compounds (Isomer 1-18 mg, 28%; Isomer 2-21 mg, 33%). Column: Chiralcel AD-H, 21×150 mm; Mobile Phase: 35% EtOH: 65% CO₂; Flow Rate: 80 mL/min; Column temperature: 40° C.; Detection: 225 nM. Retention time=0.95 min (99% ee, isomer 1) and retention time=1.36 min (98% ee, isomer 2). ES/MS (m/z): 336.0/338.0 (M+H). ¹H NMR (400 MHz, DMSO): 12.99-12.97 (m, 1H), 8.05-8.02 (m, 1H), 7.82-7.78 (m, 1H), 7.66-7.64 (m, 1H), 7.19-7.17 (m, 1H), 4.64-4.61 (m, 1H), 4.55-4.51 (m, 2H), 3.68-3.65 (m, 1H), 3.53-3.49 (m, 2H), 2.78-2.76 (m, 1H), 2.39-2.36 (m, 1H), 1.94-1.91 (m, 1H), 1.58-1.54 (m, 1H).

### Intermediate 593

### tert-Butyl (S)-(1-(4-bromo-6, 7-dichloro-1-(phenyl sulfonyl)-1H-indol-2-yl)-3-hydroxypropan-2-yl)carbamate

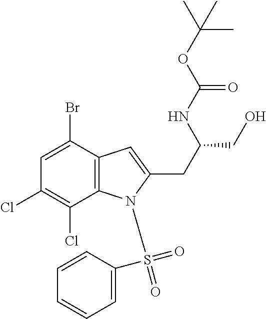 [SMILES: CC(C)(C)OC(=O)N[C@H](CO)Cc1cc2c(Br)cc(Cl)c(Cl)c2n1S(=O)(=O)c1ccccc1]

Add THF (100 mL) to 4-bromo-6,7-dichloro-1-(phenylsulfonyl)-1H-indole (5 g, 12.3 mmol) and stir under N₂ at ambient temperature until dissolved. Cool to −78° C. and add lithium diisopropylamide (2.0M in THF/heptane/ethylbenzene, 7.5 mL, 15 mmol) dropwise over 10 min. Stir at −78° C. for 45 min. Add tert-butyl 4-(((tert-butyldimethylsilyl)oxy)methyl)-1,2,3-oxathiazolidine-3-carboxylate 2,2-dioxide (CAS No. 2386255-17-8; 6.8 g, 19 mmol) in THF (30 mL) dropwise and allow to warm to ambient temperature. Stir for 5.5 hrs., and quench by the dropwise addition of hydrochloric acid (5M) in deionized water (25 mL). Stir 16-18 hrs. Pour into saturated aqueous NaHCO₃ and extract with EtOAc. Wash the combined organics with saturated aqueous NaCl, dry over MgSO₄, filter and concentrate under vacuum. Purify via flash chromatography eluting with EtOAc in hexanes to give tert-butyl (S)-(1-(4-bromo-6,7-dichloro-1-(phenylsulfonyl)-1H-indol-2-yl)-3-hydroxypropan-2-yl)carbamate (3.61 g, 51%). ES/MS (m/z): (³⁵Cl/³⁷Cl) 577.2/579.2 (M+H).

### Intermediate 594

### tert-Butyl (S)-(8-bromo-5, 6-dichloro-2,3-dihydro-1H-pyrrolo[1,2-a]indol-2-yl)carbamate

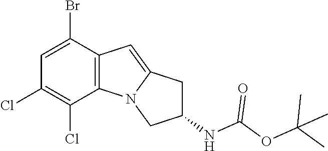 [SMILES: CC(C)(C)OC(=O)N[C@H]1Cc2cc3c(Br)cc(Cl)c(Cl)c3n2C1]

Add tetrabutylammonium fluoride (1M in THF, 22 mL, 22 mmol) tert-butyl (S)-(1-(4-bromo-6,7-dichloro-1-(phenyl sulfonyl)-1H-indol-2-yl)-3-hydroxypropan-2-yl)carbamate (3.6 g, 6.2 mmol) in THF (65 mL) with stirring, under N₂ and at ambient temperature. Heat to 80° C. for 16-18 hrs. Cool to ambient temperature, pour into saturated aqueous NH₄Cl and extract with EtOAc. Wash the combined organics with saturated aqueous NaCl, dry over MgSO₄, filter and concentrate under vacuum. Purify via flash chromatography eluting with EtOAc in hexanes to give tert-butyl (S)-(8-bromo-5,6-dichloro-2,3-dihydro-1H-pyrrolo[1,2-a]indol-2-yl)carbamate (1.5 g, 57%). ES/MS (m/z): (³⁵Cl/³⁷Cl) 419.1/421.1 (M+H).

### Intermediate 595

### Di-tert-Butyl (5,6-dichloro-2,3-dihydro-1H-pyrrolo[1,2-a]indole-2,8-diyl)(S)-dicarbamate

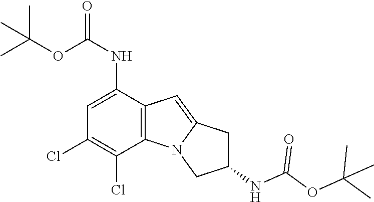 [SMILES: CC(C)(C)OC(=O)Nc1cc(Cl)c(Cl)c2c1cc1n2C[C@@H](NC(=O)OC(C)(C)C)C1]

Add 4,5-bis(diphenylphosphino)-9,9-dimethylxanthene (380 mg, 0.643 mmol) and tris(dibenzylideneacetone)dipalladium(0) (300 mg, 0.317 mmol) to tert-butyl (S)-(8-bromo-5,6-dichloro-2,3-dihydro-1H-pyrrolo[1,2-a]indol-2-yl)carbamate (1.5 g, 3.6 mmol), tert-butyl carbamate (850 mg, 7.11 mmol) and cesium carbonate (2.6 g, 8.0 mmol) in 1,4-dioxane (20 mL) with stirring, under N₂ and at ambient temperature. Degas via a stream of nitrogen for 10 min. Heat to 100° C. for 16-18 hrs. Cool to ambient temperature, pour into water and extract with EtOAc. Wash the combined organics with saturated aqueous NaCl, dry over MgSO₄, filter and concentrate under vacuum. Purify via flash chromatography eluting with EtOAc in hexanes to give di-tert-butyl (5,6-dichloro-2,3-dihydro-1H-pyrrolo[1,2-a]indole-2,8-diyl)(S)-dicarbamate (1.61 g, 99%). ES/MS (m/z): (³⁵Cl/³⁷Cl) 456.2/458.2 (M+H).

### Intermediate 596

### Di-tert-Butyl (5,6-dichloro-9-iodo-2,3-dihydro-1H-pyrrolo[1,2-a]indole-2,8-diyl)(S)-dicarbamate

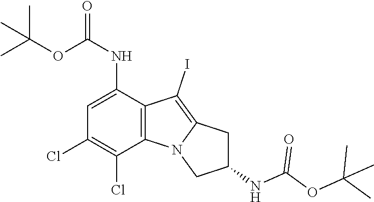 [SMILES: CC(C)(C)OC(=O)Nc1cc(Cl)c(Cl)c2c1c(I)c1n2C[C@@H](NC(=O)OC(C)(C)C)C1]

Add N-iodosuccinimide (650 mg, 2.83 mmol) to di-tert-butyl (5,6-dichloro-2,3-dihydro-1H-pyrrolo[1,2-a]indole-2,8-diyl)(S)-dicarbamate (1.3 g, 2.8 mmol) in DMF (30 mL) portion-wise with stirring, under N₂ and at ambient temperature. Stir for 1 hr. Pour into saturated aqueous Na₂S₂O₃ and extract with EtOAc. Wash the combined organic layers with water and saturated aqueous NaCl. Filter to collect solids persisting in the organic layer yielding 1.0 g of product. Dry the filtrate over MgSO₄, filter and concentrate under vacuum. Purify via flash chromatography eluting with DCM to give 430 mg (64%) of product. Combine with previously collected precipitate to give di-tert-butyl (5,6-dichloro-9-iodo-2,3-dihydro-1H-pyrrolo[1,2-a]indole-2,8-diyl)(S)-dicarbamate (1.53 g, 92%). ¹H NMR (DMSO-d6): 8.64 (s, 1H), 7.59-7.55 (m, 1H), 7.22-7.20 (m, 1H), 4.82 (dd, J=7.2, 10.7 Hz, 1H), 4.73-4.65 (m, 1H), 4.33 (dd, J=4.8, 10.7 Hz, 1H), 3.20-3.14 (m, 1H), 2.90-2.68 (m, 1H), 1.48 (s, 9H), 1.41 (s, 9H).

### Intermediate 597

### Di-tert-butyl ((2 S)-5, 6-di chloro-9-(1-(tetrahydro-2H-pyran-2-yl)-1H-pyrazol-4-yl)-2,3-dihydro-1H-pyrrolo[1,2-a]indole-2,8-diyl)dicarbamate

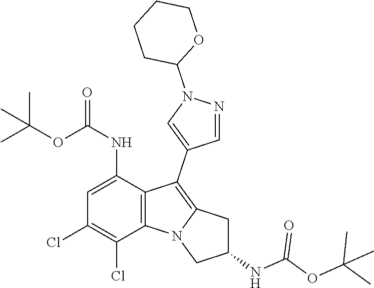 [SMILES: CC(C)(C)OC(=O)Nc1cc(Cl)c(Cl)c2c1c(-c1cnn(C3CCCCO3)c1)c1n2C[C@@H](NC(=O)OC(C)(C)C)C1]

Add sodium carbonate (820 mg, 7.74 mmol) and water (3 mL) to di-tert-butyl (5,6-dichloro-9-iodo-2,3-dihydro-1H-pyrrolo[1,2-a]indole-2,8-diyl)(S)-dicarbamate (1.5 g, 2.6 mmol), 1-(tetrahydro-2h-pyran-2-yl)-4-(4,4,5,5-tetramethyl-1,3,2-dioxaborolan-2-yl)-1h-pyrazole (1.5 g, 5.1 mmol) and Pd(dppf)Cl2 (190 mg, 0.254 mmol) in 1,4-dioxane (12 mL) with stirring and at ambient temperature. Degas via a stream of N₂ gas for 10 min. Heat to 90° C. for 5 hrs. Cool to ambient temperature, pour into water and extract with EtOAc. Wash the combined organics with saturated aqueous NaCl, dry over MgSO₄, filter and concentrate under vacuum. Purify via flash chromatography eluting with EtOAc in hexanes. Further purify recovered product by flash chromatography eluting with MeoH in DCM and EtOAc to give di-tert-butyl ((2S)-5,6-dichloro-9-(1-(tetrahydro-2H-pyran-2-yl)-1H-pyrazol-4-yl)-2,3-dihydro-1H-pyrrolo[1,2-a]indole-2,8-diyl)dicarbamate (360 mg, 23%). ES/MS (m/z): (³⁵Cl/³⁷Cl) 605.0/607.0 (M+H).

### Intermediate 598

### (S)-5,6-Dichloro-9-(1H-pyrazol-4-yl)-2,3-dihydro-1H-pyrrolo[1,2-a]indole-2, 8-diamine; dihydrochloride

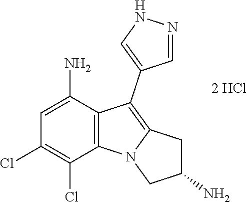 [SMILES: Nc1cc(Cl)c(Cl)c2c1c(-c1cn[nH]c1)c1n2C[C@@H](N)C1]

Add hydrochloric acid (4.0M in dioxane, 1.5 mL, 6.0 mmol) to di-tert-butyl ((2S)-5,6-dichloro-9-(1-(tetrahydro-2H-pyran-2-yl)-1H-pyrazol-4-yl)-2,3-dihydro-1H-pyrrolo[1,2-a]indole-2,8-diyl)dicarbamate (350 mg, 0.577 mmol) in 1,4-dioxane (3 mL) and methanol (1 mL) with stirring, under N₂ and at ambient temperature. After 1 hr, add an additional 5 equivalents of HCl/dioxane. When the reaction is complete, concentrate under vacuum to give (S)-5,6-dichloro-9-(1H-pyrazol-4-yl)-2,3-dihydro-1H-pyrrolo[1,2-a]indole-2,8-diamine; dihydrochloride (303 mg, used without further purification). ES/MS (m/z): (³⁵Cl/³⁷Cl) 321.8/323.8 (M+H).

### Intermediate 599

### (S)—N-(8-Amino-5,6-dichloro-9-(1H-pyrazol-4-yl)-2,3-dihydro-1H-pyrrolo[1,2-a]indol-2-yl)acetamide

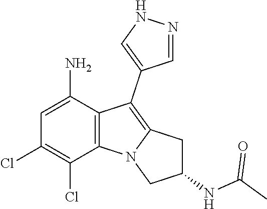 [SMILES: CC(=O)N[C@H]1Cc2c(-c3cn[nH]c3)c3c(N)cc(Cl)c(Cl)c3n2C1]

Add acetic anhydride (65 μL, 0.688 mmol) to (S)-5,6-dichloro-9-(1H-pyrazol-4-yl)-2,3-dihydro-1H-pyrrolo[1,2-a]indole-2,8-diamine; dihydrochloride (230 mg, 0.582 mmol) and TEA (800 μL, 5.74 mmol) in DCM (5 mL) with stirring, under N₂ and at ambient temperature. Stir for 2 hrs. Pour into saturated aqueous NaHCO₃ and extract with DCM. Concentrate the combined organic layers under vacuum. Triturate from Et₂O/hexanes and collect the solid by filtration. Vacuum dry to give (S)—N-(8-amino-5,6-dichloro-9-(1H-pyrazol-4-yl)-2,3-dihydro-1H-pyrrolo[1,2-a]indol-2-yl)acetamide (325 mg, 99+%). ES/MS (m/z): (³⁵Cl/³⁷Cl) 363.8/365.8 (M+H).

### EXAMPLE 244

### (S)—N-(2-Acetamido-5,6-dichloro-9-(1H-pyrazol-4-yl)-2,3-dihydro-1H-pyrrolo[1,2-a]indol-8-yl)-2,2-difluoroacetamide

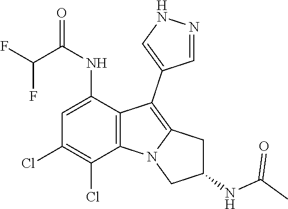 [SMILES: CC(=O)N[C@H]1Cc2c(-c3cn[nH]c3)c3c(NC(=O)C(F)F)cc(Cl)c(Cl)c3n2C1]

Dissolve (S)—N-(8-amino-5,6-dichloro-9-(1H-pyrazol-4-yl)-2,3-dihydro-1H-pyrrolo[1,2-a]indol-2-yl)acetamide (0.11 g, 0.302 mmol) in pyridine (1 mL) with stirring, under N₂ and at ambient temperature. Add difluoroacetic anhydride (15 μL, 0.453 mmol) and stir for 30 min. Add additional difluoroacetic anhydride (7.5 μL, 0.226 mmol) and stir for 30 min. Add additional anhydride (15 μL, 0.453 mmol) and stir for 60 min. Concentrate the reaction under vacuum. Dilute in EtOAc and pour into saturated aqueous NaHCO₃. Extract with EtOAc, dry the combined organics over MgSO₄, filter, and concentrate under vacuum. Purify by prep-HPLC to give (S)—N-(2-acetamido-5,6-dichloro-9-(1H-pyrazol-4-yl)-2,3-dihydro-1H-pyrrolo[1,2-a]indol-8-yl)-2,2-difluoroacetamide (23 mg, 17%), ES/MS (m/z): (³⁵Cl/³⁷Cl) 442.0/444.0 (M+H).

### Intermediate 600

### (S)-8-Bromo-5,6-dichloro-9-(1H-pyrazol-4-yl)-2,3-dihydro-1H-pyrrolo[1,2-a]indol-2-amine 2,2,2-trifluoroacetate

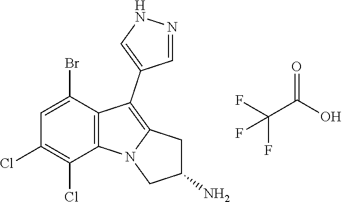 [SMILES: N[C@H]1Cc2c(-c3cn[nH]c3)c3c(Br)cc(Cl)c(Cl)c3n2C1 ; O=C(O)C(F)(F)F]

Add TFA (1.5 mL, 20 mmol) to tert-butyl (S)-(8-bromo-5,6-dichloro-2,3-dihydro-1H-pyrrolo[1,2-a]indol-2-yl)carbamate (735 mg, 1.75 mmol) in DCM (6 mL) with stirring, under N₂ and at ambient temperature. Stir for 1 hr. Concentrate under vacuum to give (S)-8-bromo-5,6-dichloro-9-(1H-pyrazol-4-yl)-2,3-dihydro-1H-pyrrolo[1,2-a]indol-2-amine 2,2,2-trifluoroacetate (600 mg, used without purification). ES/MS (m/z): (³⁵Cl/³⁷Cl) 320.8/318.8 (M+H).

### Intermediate 601

### (S)—N-(8-Bromo-5,6-dichloro-9-(1H-pyrazol-4-yl)-2,3-dihydro-1H-pyrrolo[1,2-a]indol-2-yl)acetamide

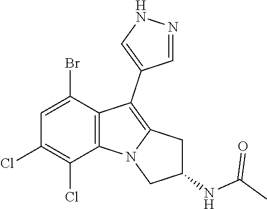 [SMILES: CC(=O)N[C@H]1Cc2c(-c3cn[nH]c3)c3c(Br)cc(Cl)c(Cl)c3n2C1]

Add acetyl chloride (160 μL, 2.25 mmol) to (S)-8-bromo-5,6-dichloro-9-(1H-pyrazol-4-yl)-2,3-dihydro-1H-pyrrolo[1,2-a]indol-2-amine 2,2,2-trifluoroacetate (600 mg, 1.87 mmol) and TEA (1.3 mL, 9.3 mmol) in THF (10 mL) with stirring, under N₂ and at 0° C. Continue stirring with gradual warming to ambient temperature for 1 hr. Pour into water and extract with EtOAc. Wash combined organics with saturated aqueous NaCl, dry over MgSO₄, filter and concentrate under vacuum. Purify via flash chromatography eluting with acetone in DCM to give (S)—N-(8-bromo-5,6-dichloro-9-(1H-pyrazol-4-yl)-2,3-dihydro-1H-pyrrolo[1,2-a]indol-2-yl)acetamide (552 mg, 81%). ES/MS (m/z): (³⁵Cl/³⁷Cl) 362.6/364.6 (M+H).

### Intermediate 602

### (S)—N-(5,6-Dichloro-8-hydroxy-9-(1H-pyrazol-4-yl)-2,3-dihydro-1H-pyrrolo[1,2-a]indol-2-yl)acetamide

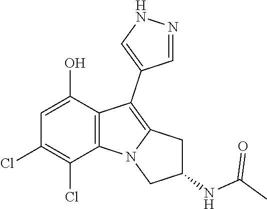 [SMILES: CC(=O)N[C@H]1Cc2c(-c3cn[nH]c3)c3c(O)cc(Cl)c(Cl)c3n2C1]

Add sodium tert-butoxide (365 mg, 3.8 mmol) and water (3 mL) to (S)—N-(8-bromo-5,6-dichloro-9-(1H-pyrazol-4-yl)-2,3-dihydro-1H-pyrrolo[1,2-a]indol-2-yl)acetamide (550 mg, 1.52 mmol) and tBuBrettPhos Pd G3 (135 mg, 0.151 mmol) in 1,4-dioxane (10 mL) with stirring, under N₂ and at ambient temperature. Heat to 65° C. for 3 hrs. Cool to ambient temperature, pour into saturated aqueous NH₄Cl and extract with EtOAc. Wash the combined organics with saturated aqueous NaCl, dry over MgSO₄, filter and concentrate under vacuum. Purify via flash chromatography eluting with EtOAc in hexanes to give (S)—N-(5,6-dichloro-8-hydroxy-9-(1H-pyrazol-4-yl)-2,3-dihydro-1H-pyrrolo[1,2-a]indol-2-yl)acetamide (335 mg, 74%). ES/MS (m/z): (³⁵Cl/³⁷Cl) 299.0/301.0 (M+H).

### Intermediate 603

### (S)—N-(5,6-Dichloro-8-(cyanomethoxy)-2,3-dihydro-1H-pyrrolo[1,2-a]indol-2-yl)acetamide

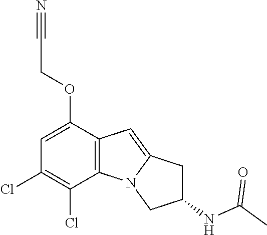 [SMILES: CC(=O)N[C@H]1Cc2cc3c(OCC#N)cc(Cl)c(Cl)c3n2C1]

Add bromoacetonitrile (120 μL, 1.72 mmol) to (S)—N-(5,6-dichloro-8-hydroxy-9-(1H-pyrazol-4-yl)-2,3-dihydro-1H-pyrrolo[1,2-a]indol-2-yl)acetamide (335 mg, 1.12 mmol) and potassium carbonate (165 mg, 1.19 mmol) in DMF (6 mL) with stirring, under N₂ and at ambient temperature. After 2 hrs., pour into water and extract with EtOAc. Wash the combined organics with water and saturated aqueous NaCl, dry over MgSO₄, filter and concentrate under vacuum. Triturate with Et₂O/hexanes, collect via filtration, and vacuum dry to give (S)—N-(5,6-dichloro-8-(cyanomethoxy)-2,3-dihydro-1H-pyrrolo[1,2-a]indol-2-yl)acetamide (324 mg, 86%). ES/MS (m/z): (³⁵Cl/³⁷Cl) 337.8/339.8 (M+H).

### Intermediate 604

### (S)—N-(5,6-Dichloro-8-(cyanomethoxy)-9-iodo-2,3-dihydro-1H-pyrrolo[1,2-a]indol-2-yl)acetamide

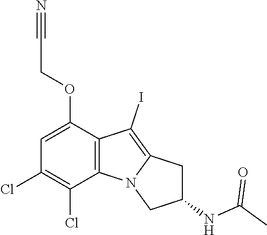 [SMILES: CC(=O)N[C@H]1Cc2c(I)c3c(OCC#N)cc(Cl)c(Cl)c3n2C1]

Add N-iodosuccinimide (240 mg, 1.04 mmol) to (S)—N-(5,6-dichloro-8-(cyanomethoxy)-2,3-dihydro-1H-pyrrolo[1,2-a]indol-2-yl)acetamide (320 mg, 0.946 mmol) in DMF (10 mL) with stirring, under N₂ and at ambient temperature. After 1 hr., pour into saturated aqueous Na₂S₂O₃ and extract with EtOAc. Wash the combined organics with water and saturated aqueous NaCl, dry over MgSO₄, filter, and concentrate under vacuum. Triturate from Et₂O/hexanes, collect the solid by filtration and dry under vacuum to give (S)—N-(5,6-dichloro-8-(cyanomethoxy)-9-iodo-2,3-dihydro-1H-pyrrolo[1,2-a]indol-2-yl)acetamide (405 mg, 92%) ES/MS (m/z): (³⁵Cl/³⁷Cl) 463.6/465.6 (M+H).

### Intermediate 605

### N-((2 S)-5, 6-Dichloro-8-(cyanomethoxy)-9-(1-(tetrahydro-2H-pyran-2-yl)-1H-pyrazol-4-yl)-2,3-dihydro-1H-pyrrolo[1,2-a]indol-2-yl)acetamide

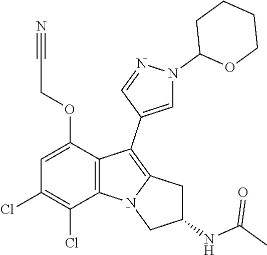 [SMILES: CC(=O)N[C@H]1Cc2c(-c3cnn(C4CCCCO4)c3)c3c(OCC#N)cc(Cl)c(Cl)c3n2C1]

Add potassium carbonate (60 mg, 0.434 mmol), ACN (1 mL) and water (350 μl) to tri-tert-butylphosphonium tetrafluoroborate (7 mg, 0.023 mmol) and crotylpalladium chloride dimer (3 mg, 0.007 mmol) with stirring, under N₂ and at ambient temperature. After 30 min, transfer mixture to a second flask containing (S)—N-(5,6-dichloro-8-(cyanomethoxy)-9-iodo-2,3-dihydro-1H-pyrrolo[1,2-a]indol-2-yl)acetamide (100 mg, 0.215 mmol) and 1-(tetrahydro-2h-pyran-2-yl)-4-(4,4,5,5-tetramethyl-1,3,2-dioxaborolan-2-yl)-1h-pyrazole (76 mg, 0.267 mmol). Heat to 50° C. for 2 hrs. Pour into water and extract with EtOAc. Wash the combined organics with saturated aqueous NaCl, dry over MgSO₄, filter and concentrate under vacuum. Purify by reverse phase chromatography to give N-((2S)-5,6-dichloro-8-(cyanomethoxy)-9-(1-(tetrahydro-2H-pyran-2-yl)-1H-pyrazol-4-yl)-2,3-dihydro-1H-pyrrolo[1,2-a]indol-2-yl)acetamide (55 mg, 52%) ES/MS (m/z): (³⁵Cl/³⁷Cl) 487.8/489.8 (M+H).

### EXAMPLE 245

### (S)—N-(5,6-Dichloro-8-(cyanomethoxy)-9-(1H-pyrazol-4-yl)-2,3-dihydro-1H-pyrrolo[1,2-a]indol-2-yl)acetamide

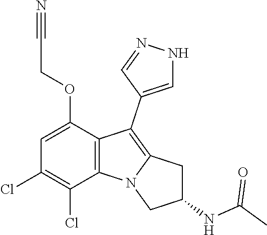 [SMILES: CC(=O)N[C@H]1Cc2c(-c3cn[nH]c3)c3c(OCC#N)cc(Cl)c(Cl)c3n2C1]

Suspend N-((2S)-5,6-dichloro-8-(cyanomethoxy)-9-(1-(tetrahydro-2H-pyran-2-yl)-1H-pyrazol-4-yl)-2,3-dihydro-1H-pyrrolo[1,2-a]indol-2-yl)acetamide (50 mg, 0.102 mmol) in DCM (2 mL) with stirring, under N₂ and at ambient temperature. Add TFA (100 μL, 1.02 mmol). After 1 hr., add TFA (100 μL, 1.02 mmol). Concentrate via a stream of N₂ gas. Dilute in EtOAc and pour into saturated aqueous NaHCO₃. Extract with EtOAc, dry the combined organics over MgSO₄, filter, and concentrate under vacuum. Purify by prep-HPLC to give (S)—N-(5,6-dichloro-8-(cyanomethoxy)-9-(1H-pyrazol-4-yl)-2,3-dihydro-1H-pyrrolo[1,2-a]indol-2-yl)acetamide (26 mg, 63%). ES/MS (m/z): (³⁵Cl/³⁷Cl) 403.8/405.8 (M+H). ¹H NMR (DMSO-d6): 12.90-12.87 (s, 1H), 8.47 (d, J=6.5 Hz, 1H), 7.92-7.89 (m, 2H), 6.96 (s, 1H), 5.28 (s, 2H), 4.90-4.85 (m, 1H), 4.74-4.70 (m, 1H), 4.35 (dd, J=3.8, 11.1 Hz, 1H), 3.44-3.37 (m, 1H), 2.90 (dd, J=3.9, 16.8 Hz, 1H), 1.83 (s, 3H).

### Intermediate 606

### Ethyl 2-(6,7-dichloroindol-1-yl)acetate

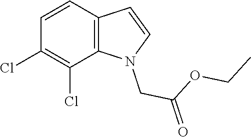 [SMILES: CCOC(=O)Cn1ccc2ccc(Cl)c(Cl)c21]

Add together 6,7-dichloro-1H-indole (1.96 g, 10.4 mmol), DMF (50.0 mL), cesium carbonate (3.72 g, 11.4 mmol) and ethyl bromoacetate (1.30 mL, 11.7 mmol). Heat the mixture to 50° C. Stir for 12 hrs. Add another 500 μL of ethyl bromoacetate and continue heating for 10 hrs. Quench with water and extract with EtOAc. Wash the combined organics with water and saturated aqueous NaCl, dry over MgSO₄, filter, and concentrate under vacuum. Purify by flash column chromatography eluting with EtOAc in hexanes to give ethyl 2-(6,7-dichloroindol-1-yl)acetate (1.87 g, 66%). ¹H NMR (400 MHz, DMSO-d6): 7.56 (d, J=8.4 Hz, 1H), 7.44 (d, J=3.2 Hz, 1H), 7.24 (d, J=8.4 Hz, 1H), 6.57 (d, J=3.1 Hz, 1H), 5.38 (s, 2H), 4.17 (q, J=7.1 Hz, 2H), 1.21 (t, J=7.1 Hz, 3H).

### Intermediate 607

### Ethyl 2-(3-bromo-6,7-dichloro-indol-1-yl)acetate

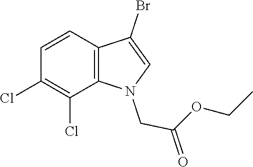 [SMILES: CCOC(=O)Cn1cc(Br)c2ccc(Cl)c(Cl)c21]

Mix together ethyl 2-(6,7-dichloroindol-1-yl)acetate (1.87 g, 6.87 mmol), DMF (35.0 mL), and N-bromosuccinimide (1.35 g, 7.58 mmol) and stir at ambient temperature for 19 h. Quench with water when reaction is complete. Extract with EtOAc, wash the combined organics with water and saturated aqueous NaCl, dry over MgSO₄, filter, and concentrate under vacuum. Purify by flash column chromatography eluting with EtOAc in hexanes then with MTBE in hexanes to give ethyl 2-(3-bromo-6,7-dichloro-indol-1-yl)acetate (2.10 g, 81%). ES/MS m/z: 366.6/368.6/370.6 (M+NH₄).

### Intermediate 608

### Ethyl 2-[6,7-dichloro-3-(1-tetrahydropyran-2-ylpyrazol-4-yl)indol-1-yl]acetate

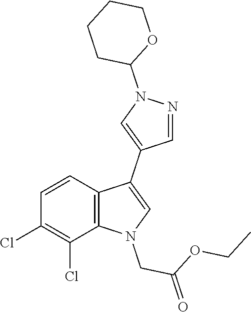 [SMILES: CCOC(=O)Cn1cc(-c2cnn(C3CCCCO3)c2)c2ccc(Cl)c(Cl)c21]

Prepare a mixture of ethyl 2-(3-bromo-6,7-dichloro-indol-1-yl)acetate (5.27 g, 15.0 mmol), Pd(dppf)Cl₂ (2.3 g, 3.0 mmol), 1,2-dimethoxyethane (100 mL), 1-tetrahydropyran-2-yl-4-(4,4,5,5-tetramethyl-1,3,2-dioxaborolan-2-yl)pyrazole (8.35 g, 30.0 mmol), and potassium phosphate tribasic (45 mL, 45 mmol) and degas under N₂. Heat to 80° C. for 2 hrs. Concentrate, dilute with EtOAc, wash with water and saturated aqueous NaCl, dry over MgSO₄, filter, and concentrate under vacuum. Purify by flash column chromatography eluting with EtOAc in hexanes to give ethyl 2-[6,7-dichloro-3-(1-tetrahydropyran-2-ylpyrazol-4-yl)indol-1-yl]acetate (4.00 g, 63%). ES/MS (m/z): (³⁵Cl/³⁷Cl) 422.0/424.0 (M+H).

### Intermediate 609

### 2-[6,7-Dichloro-3-(1-tetrahydropyran-2-ylpyrazol-4-yl)indol-1-yl]acetic acid

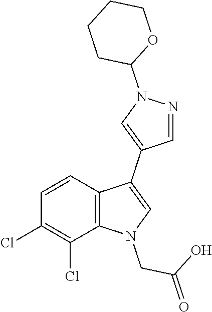 [SMILES: O=C(O)Cn1cc(-c2cnn(C3CCCCO3)c2)c2ccc(Cl)c(Cl)c21]

Mix together ethyl 2-[6,7-dichloro-3-(1-tetrahydropyran-2-ylpyrazol-4-yl)indol-1-yl]acetate (4.00 g, 9.47 mmol), THF (40.0 mL), MeOH (40.0 mL), water (20.0 mL) and lithium hydroxide (0.700 g, 29.2 mmol). Stir the mixture for 2 hrs. and concentrate under vacuum. Acidify with 1M HCl and quickly extract with copious amounts of EtOAc. Wash the combined organics with saturated aqueous NaCl and dry over MgSO₄, filter, and concentrate under vacuum to give 2-[6,7-dichloro-3-(1-tetrahydropyran-2-ylpyrazol-4-yl)indol-1-yl]acetic acid (3.10 g, 83%). ES/MS (m/z): (³⁵Cl/³⁷Cl) 394.0/396.0 (M+H).

### Intermediate 610

### (4-Nitrophenyl) 2-[6, 7-dichloro-3-(1-tetrahydropyran-2-ylpyrazol-4-yl)indol-1-yl]acetate

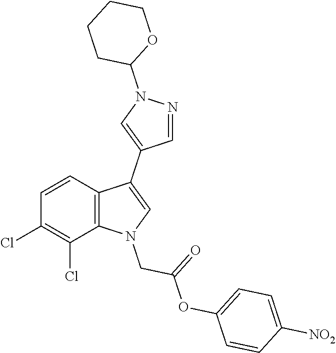 [SMILES: O=C(Cn1cc(-c2cnn(C3CCCCO3)c2)c2ccc(Cl)c(Cl)c21)Oc1ccc([N+](=O)[O-])cc1]

Mix 2-[6, 7-dichloro-3-(1-tetrahydropyran-2-ylpyrazol-4-yl)indol-1-yl]acetic acid (3.10 g, 7.86 mmol), 4-nitrophenol (1.35 g, 9.41 mmol), N,N′-dicyclohexylcarbodiimide (1.95 g, 9.45 mmol), and THF (80.0 mL, 983 mmol). Stir the mixture for 72 hrs. Concentrate under vacuum and purify by column chromatography eluting with EtOAc in DCM to give (4-nitrophenyl) 2-[6,7-dichloro-3-(1-tetrahydropyran-2-ylpyrazol-4-yl)indol-1-yl]acetate (2.86 g, 71%). ES/MS (m/z): (³⁵Cl/³⁷Cl) 515.0/517.0 (M+H).

### Intermediate 611

### 1-[6,7-Dichloro-3-(1-tetrahydropyran-2-ylpyrazol-4-yl)indol-1-yl]-3-[dimethyl(oxo)-λ⁶-sulfanylidene]propan-2-one

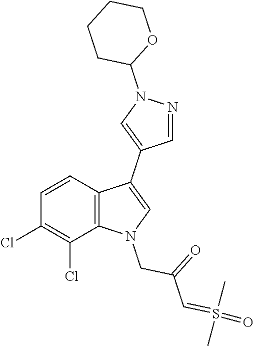 [SMILES: CS(C)(=O)=CC(=O)Cn1cc(-c2cnn(C3CCCCO3)c2)c2ccc(Cl)c(Cl)c21]

Mix trimethylsulfoxonium iodide (1.80 g, 8.18 mmol), DMF (25 mL) and potassium tert-butoxide (0.93 g, 8.2 mmol). Stir for a few minutes and add a solution of (4-nitrophenyl) 2-[6,7-dichloro-3-(1-tetrahydropyran-2-ylpyrazol-4-yl)indol-1-yl]acetate (2.64 g, 5.12 mmol) in DMF (25 mL) at ambient temperature. Stir for 16-18 hrs. Cool mixture in an ice bath and quench with water. Extract with EtOAc, wash the organic layer with water and saturated aqueous NaCl, dry over anhydrous MgSO₄, filter, and concentrate under vacuum. Purify by column chromatography eluting with MeOH in DCM to give 1-[6,7-dichloro-3-(1-tetrahydropyran-2-ylpyrazol-4-yl)indol-1-yl]-3-[dimethyl(oxo)-λ⁶-sulfanylidene]propan-2-one (1.32 g, 55%). 1H NMR (400.13 MHz, DMSO): 8.26 (s, 1H), 7.84 (s, 1H), 7.80 (d, J=8.5 Hz, 1H), 7.65 (s, 1H), 7.27 (d, J=8.5 Hz, 1H), 5.44 (dd, J=2.2, 10.1 Hz, 1H), 5.01 (s, 2H), 4.50 (s, 1H), 3.99-3.92 (m, 1H), 3.71-3.59 (m, 1H), 3.43 (s, 6H), 2.26-2.14 (m, 1H), 2.02-1.90 (m, 2H), 1.76-1.63 (m, 1H), 1.60-1.52 (m, 2H).

### Intermediate 612

### 7,8-Dichloro-4-(1-tetrahydropyran-2-ylpyrazol-4-yl)-1,3-dihydropyrrolo[1,2-a]indol-2-one

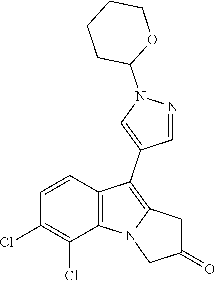 [SMILES: O=C1Cc2c(-c3cnn(C4CCCCO4)c3)c3ccc(Cl)c(Cl)c3n2C1]

Add 1-[6,7-dichloro-3-(1-tetrahydropyran-2-ylpyrazol-4-yl)indol-1-yl]-3-[dimethyl(oxo)-λ⁶-sulfanylidene]propan-2-one (1.32 g, 2.82 mmol), 1,2-dichloroethane (15.0 mL) and chloro(1,5-cyclooctadiene)iridium(I) dimer (100 mg, 0.144 mmol, 97) to a microwave vial. Heat in a microwave reactor at 80° C. for 3 hrs. Concentrate under vacuum. Purify by reverse phase chromatography to give 7,8-dichloro-4-(1-tetrahydropyran-2-ylpyrazol-4-yl)-1,3-dihydropyrrolo[1,2-a]indol-2-one (358 mg, 32%). ES/MS (m/z): (³⁵Cl/³⁷Cl) 388.0/390.0 (M−H).

### Intermediate 613

### 5, 6-Dichloro-9-(1-(tetrahydro-2H-pyran-2-yl)-1H-pyrazol-4-yl)-2,3-dihydro-1H-pyrrolo[1,2-a]indol-2-amine

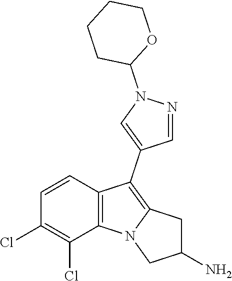 [SMILES: NC1Cc2c(-c3cnn(C4CCCCO4)c3)c3ccc(Cl)c(Cl)c3n2C1]

Add sodium cyanoborohydride (100 mg, 1.59 mmol) to 7,8-dichloro-4-(1-tetrahydropyran-2-ylpyrazol-4-yl)-1,3-dihydropyrrolo[1,2-a]indol-2-one (0.358 g, 0.917 mmol), ammonium acetate (0.095 g, 1.23 mmol) and acetic acid (50 μl, 0.917 mmol) in MeOH (5 mL) with stirring, under N₂ and at ambient temperature. Stir for 16-18 hrs. Pour into saturated NaHCO₃ and extract with EtOAc. Dry the combined organics over MgSO₄, filter, and concentrate under vacuum. Purify by reverse phase chromatography to give 5,6-dichloro-9-(1-(tetrahydro-2H-pyran-2-yl)-1H-pyrazol-4-yl)-2,3-dihydro-1H-pyrrolo[1,2-a]indol-2-amine (25 mg, 7%) ES/MS (m/z): (³⁵Cl/³⁷Cl) 391.2/393.2 (M+H).

### Intermediate 614

### N-(5,6-Dichloro-9-(1-(tetrahydro-2H-pyran-2-yl)-1H-pyrazol-4-yl)-2,3-dihydro-1H-pyrrolo[1,2-a]indol-2-yl)methanesulfonamide

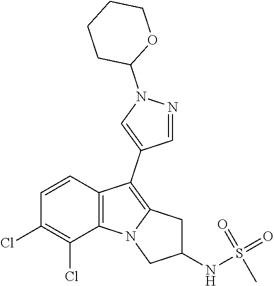 [SMILES: CS(=O)(=O)NC1Cc2c(-c3cnn(C4CCCCO4)c3)c3ccc(Cl)c(Cl)c3n2C1]

Add methanesulfonyl chloride (0.05 μL, 0.064 mmol) to 5,6-dichloro-9-(1-(tetrahydro-2H-pyran-2-yl)-1H-pyrazol-4-yl)-2,3-dihydro-1H-pyrrolo[1,2-a]indol-2-amine (0.025 g, 0.061 mmol) and TEA (13 μL, 0.093 mmol) in DCM (1 mL) with stirring, under N₂ and at 0° C. Stir for 5 min, remove the ice bath and continue stirring at ambient temperature for 1 hr. Pour into water and extract with DCM. Wash the combined organics with saturated aqueous NaCl, dry over MgSO₄, filter and concentrate to give crude N-(5,6-dichloro-9-(1-(tetrahydro-2H-pyran-2-yl)-1H-pyrazol-4-yl)-2,3-dihydro-1H-pyrrolo[1,2-a]indol-2-yl)methanesulfonamide (25 mg, 83%). ES/MS (m/z): (³⁵Cl/³⁷Cl) 469.0/471.0 (M+H).

### EXAMPLE 246

### N-(5,6-Dichloro-9-(1H-pyrazol-4-yl)-2,3-dihydro-1H-pyrrolo[1,2-a]indol-2-yl)methanesulfonamide

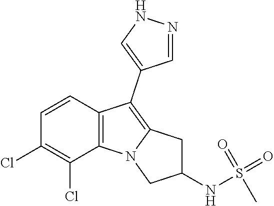 [SMILES: CS(=O)(=O)NC1Cc2c(-c3cn[nH]c3)c3ccc(Cl)c(Cl)c3n2C1]

Add hydrochloric acid (4M) in dioxane (130 μL, 0.517 mmol) to N-(5,6-dichloro-9-(1-(tetrahydro-2H-pyran-2-yl)-1H-pyrazol-4-yl)-2,3-dihydro-1H-pyrrolo[1,2-a]indol-2-yl)methanesulfonamide (25 mg, 0.0517 mmol) in 1,4-dioxane (1.5 mL) and MeOH (0.5 mL) with stirring, under N₂ and at ambient temperature. After 1 hr., add additional HCl/MeOH and continue stirring at ambient temperature. After an additional 1 hr., concentrate the reaction under vacuum. Take up the residue in EtOAc and wash with saturated aqueous NaHCO₃. Dry the organics over MgSO₄, filter, and concentrate under vacuum. Purify by prep-HPLC to give N-(5,6-dichloro-9-(1H-pyrazol-4-yl)-2,3-dihydro-1H-pyrrolo[1,2-a]indol-2-yl)methanesulfonamide (6.8 mg, 33%). ES/MS (m/z): (³⁵Cl/³⁷Cl) 385.0/387.0 (M+H). ¹H NMR (DMSO-d6): 13.04-13.02 (s, 1H), 8.17-8.13 (s, 1H), 7.88-7.79 (m, 2H), 7.73 (d, J=8.5 Hz, 1H), 7.22 (d, J=8.5 Hz, 1H), 4.86 (dd, J=7.3, 10.6 Hz, 1H), 4.76-4.71 (m, 1H), 4.30 (dd, J=5.6, 10.7 Hz, 1H), 3.61-3.55 (m, 1H), 3.08 (m, 4H).

### EXAMPLES 247 & 248

### N-(5,6-Dichloro-9-(1H-pyrazol-4-yl)-2,3-dihydro-1H-pyrrolo[1,2-a]indol-2-yl)-2-hydroxyacetamide

### Isomer 1 and Isomer 2

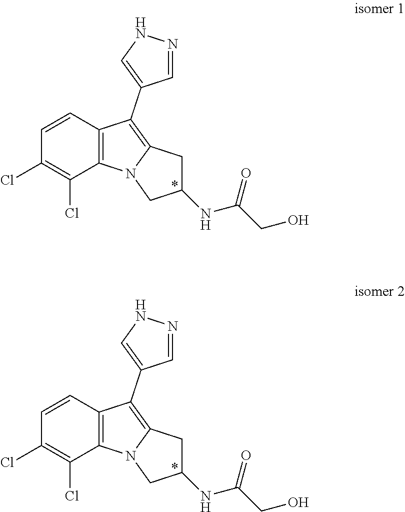 [SMILES: O=C(CO)NC1Cc2c(-c3cn[nH]c3)c3ccc(Cl)c(Cl)c3n2C1 ; O=C(CO)NC1Cc2c(-c3cn[nH]c3)c3ccc(Cl)c(Cl)c3n2C1]

Purify N-[5,6-dichloro-9-(1H-pyrazol-4-yl)-2,3-dihydro-1H-pyrrolo[1,2-a]indol-2-yl]-2-hydroxyacetamide (0.47 g, 0.128 mmol) by chiral supercritical fluid chromatography to give the N-(5,6-dichloro-9-(1H-pyrazol-4-yl)-2,3-dihydro-1H-pyrrolo[1,2-a]indol-2-yl)-2-hydroxyacetamide (Isomer 1-19 mg, 39%); N-(5,6-dichloro-9-(1H-pyrazol-4-yl)-2,3-dihydro-1H-pyrrolo[1,2-a]indol-2-yl)-2-hydroxyacetamide (Isomer 2-18 mg, 37%). Retention time=1.80 min (98.4% ee, isomer 1) and retention time=2.58 min (95.6% ee, isomer 2). Column: Chiralpak AS-H, 21×250 mm; Mobile Phase: 40% EtOH: 60% CO₂; Flow Rate: 80 mL/min; Column temperature: 40° C.; Detection: 225 nM. ES/MS (m/z): (³⁵Cl/³⁷Cl) 365.0/367.0 (M+H). 1H NMR (DMSO-d6): 13.00-12.92 (s, 1H), 8.43 (d, J=7.4 Hz, 1H), 8.12-8.04 (s, 1H), 7.88-7.85 (s, 1H), 7.73 (d, J=8.6 Hz, 1H), 7.21 (d, J=8.5 Hz, 1H), 5.46 (t, J=5.9 Hz, 1H), 5.11-5.02 (m, 1H), 4.76 (m, 1H), 4.36 (dd, J=5.0, 10.6 Hz, 1H), 3.86 (d, J=5.8 Hz, 2H), 3.49-3.43 (m, 1H), 3.13 (dd, J=5.3, 16.6 Hz, 1H).

### Intermediate 615

### (6-Bromo-3,4-dichloro-2-fluoro-phenyl)-(1-tetrahydropyran-2-ylpyrazol-4-yl)methanol

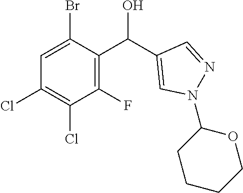 [SMILES: OC(c1cnn(C2CCCCO2)c1)c1c(Br)cc(Cl)c(Cl)c1F]

Dissolve 5-bromo-1,2-dichloro-3-fluorobenzene (11.0 g, 42.8 mmol) in THF (143 mL) and cool to −78° C. Add LDA (27 mL, 54 mmol, 2.0M in THF/heptane/ethylbenzene) dropwise over 30 minutes. Stir the solution for 1 hr. at −78° C. Add a solution of 1-tetrahydropyran-2-ylpyrazole-4-carbaldehyde (8.77 g, 42.8 mmol) in THF (34 mL) dropwise over 30 minutes. Stir the solution for 1 hr. at −78° C. Pour the reaction mixture into saturated aqueous NH₄Cl and then dilute with EtOAc. Separate the layers and wash the organic layer sequentially with saturated aqueous NH₄Cl and saturated aqueous NaCl, dry over anhydrous sodium sulfate, filter, and concentrate under vacuum. Dissolve the residue in DCM and concentrate under vacuum to give (6-bromo-3,4-dichloro-2-fluoro-phenyl)-(1-tetrahydropyran-2-ylpyrazol-4-yl)methanol (19.44 g, 96%) as a yellowish-orange solid. ES/MS (m/z): 422.8/424.8/426.8 (M+H).

### Intermediate 616

### (6-Bromo-3,4-dichloro-2-fluoro-phenyl)-(1-tetrahydropyran-2-ylpyrazol-4-yl)m ethanone

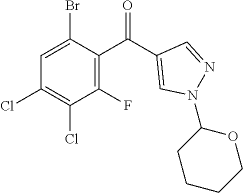 [SMILES: O=C(c1cnn(C2CCCCO2)c1)c1c(Br)cc(Cl)c(Cl)c1F]

Suspend (6-bromo-3,4-dichloro-2-fluoro-phenyl)-(1-tetrahydropyran-2-ylpyrazol-4-yl)methanol (19.44 g, 41.25 mmol) and manganese dioxide (42.2 g, 413 mmol) in DCM (412 mL). Stir at ambient temperature for 17 hrs. Filter through diatomaceous earth and wash the filter cake twice with DCM and twice with EtOAc. Concentrate the filtrate under vacuum. Purify by flash column chromatography eluting with EtOAc in hexanes to give (6-bromo-3,4-dichloro-2-fluoro-phenyl)-(1-tetrahydropyran-2-ylpyrazol-4-yl)methanone (18.67 g, 96%) as an orange solid. ES/MS (m/z): 421.0/423.0/425.0 (M+H).

### Intermediate 617

### 4-Bromo-6,7-dichloro-3-(1-tetrahydropyran-2-ylpyrazol-4-yl)-1H-indazole

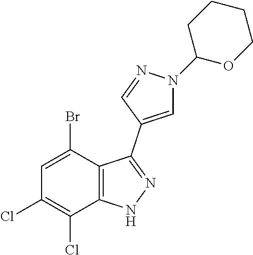 [SMILES: Clc1cc(Br)c2c(-c3cnn(C4CCCCO4)c3)n[nH]c2c1Cl]

Dissolve (6-bromo-3,4-dichloro-2-fluoro-phenyl)-(1-tetrahydropyran-2-ylpyrazol-4-yl)methanone (10 g, 23.7 mmol) in 1,4-dioxane (50 mL). Add hydrazine hydrate (11.86 mL, 237 mmol) dropwise. Stir the solution for 3 hrs. at 100° C. Cool to ambient temperature and pour into 250 mL of water. Collect the solid by filtration and wash with water and then with MeOH. Dry under vacuum to give 4-bromo-6,7-dichloro-3-(1-tetrahydropyran-2-ylpyrazol-4-yl)-1H-indazole (9.6 g, 97%) as a white solid. ES/MS (m/z): 414.8/416.8/418.8 (M+H).

### Intermediate 618A

### 4-Bromo-6,7-dichloro-1-tetrahydropyran-2-yl-3-(1-tetrahydropyran-2-ylpyrazol-4-yl)indazole

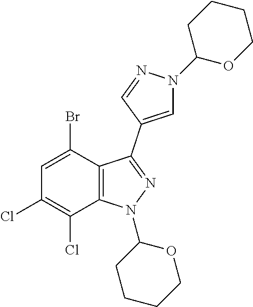 [SMILES: Clc1cc(Br)c2c(-c3cnn(C4CCCCO4)c3)nn(C3CCCCO3)c2c1Cl]

Combine 4-bromo-6,7-dichloro-3-(1-tetrahydropyran-2-ylpyrazol-4-yl)-1H-indazole (1.51 g, 3.63 mmol), THF (18.1 mL), 3,4-dihydro-2H-pyran (0.67 mL, 7.2 mmol), and methanesulfonic acid (48 μL, 0.73 mmol) and heat to 60° C. for 2 hrs. Cool to ambient temperature and add the reaction mixture slowly to a stirring mixture of saturated aqueous NaHCO₃ and EtOAc. Separate the layers, wash the organic layer with saturated aqueous NaCl, dry over anhydrous sodium sulfate, filter, and concentrate under vacuum. Purify by flash column chromatography eluting with EtOAc in hexanes to give 4-bromo-6,7-dichloro-1-tetrahydropyran-2-yl-3-(1-tetrahydropyran-2-ylpyrazol-4-yl)indazole (761 mg, 42%) as a white solid. ES/MS (m/z): 499.0/501.0/503.0 (M+H). ¹H NMR (DMSO-d6): 8.21 (s, 1H), 7.77 (s, 1H), 7.76 (s, 1H), 6.34 (dd, J=1.9, 9.7 Hz, 1H), 5.49 (dd, J=2.0, 9.9 Hz, 1H), 4.00-3.86 (m, 2H), 3.78-3.70 (m, 1H), 3.70-3.61 (m, 1H), 2.51-2.38 (m, 1H), 2.22-1.91 (m, 5H), 1.81-1.63 (m, 2H), 1.62-1.48 (m, 4H).

### Intermediate 618B

### 4-Bromo-6,7-dichloro-2-tetrahydropyran-2-yl-3-(1-tetrahydropyran-2-ylpyrazol-4-yl)indazole

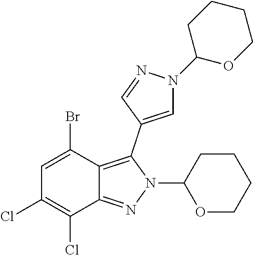 [SMILES: Clc1cc(Br)c2c(-c3cnn(C4CCCCO4)c3)n(C3CCCCO3)nc2c1Cl]

Elute the column from the purification of intermediate 618A further to give 4-bromo-6,7-dichloro-2-tetrahydropyran-2-yl-3-(1-tetrahydropyran-2-ylpyrazol-4-yl)indazole as a white solid (859 mg, 1.68 mmol, 46%, 1:1 mixture of relative stereoisomers). ES/MS (m/z): 499.0/501.0/503.0 (M+H) and 415.0/416.8/419.0 (M+H-THP). ¹H NMR (DMSO-d6): 8.24 (s, 0.5H), 8.23 (s, 0.5H), 7.77 (s, 0.5H), 7.77 (s, 0.5H), 7.55 (s, 0.5H), 7.54 (s, 0.5H), 5.59-5.52 (m, 1H), 5.40 (dd, J=9.9, 2.4 Hz, 0.5H), 5.37 (dd, J=9.9, 2.4 Hz, 0.5H), 4.02-3.93 (m, 2H), 3.73-3.64 (m, 1H), 3.55-3.47 (m, 1H), 2.50-2.39 (m, 1H), 2.20-2.09 (m, 1H), 2.07-1.91 (m, 4H), 1.78-1.49 (m, 6H).

### Intermediate 619

### 6,7-Dichloro-1-tetrahydropyran-2-yl-3-(1-tetrahydropyran-2-ylpyrazol-4-yl)indazol-4-ol

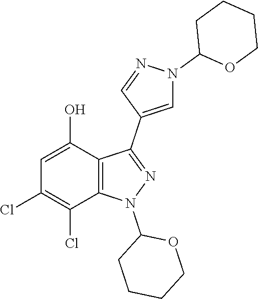 [SMILES: Oc1cc(Cl)c(Cl)c2c1c(-c1cnn(C3CCCCO3)c1)nn2C1CCCCO1]

Combine 4-bromo-6,7-dichloro-1-tetrahydropyran-2-yl-3-(1-tetrahydropyran-2-ylpyrazol-4-yl)indazole (427 mg, 0.768 mmol), cesium hydroxide hydrate (323 mg, 1.92 mmol), tBuBrettPhos Pd G3 (68 mg, 0.76 mmol), 1,4-dioxane (6.4 mL), and water (3.1 mL) under N₂ and heat to 65° C. for 2 hrs. Cool to ambient temperature, add acetic acid until pH 7, filter through silica gel, and rinse the silica gel with EtOAc. Wash the organic layer sequentially with water and saturated aqueous NaCl, dry over anhydrous sodium sulfate, filter, and concentrate under vacuum. Purify by flash column chromatography eluting with EtOAc in hexanes. Dissolve the product in DCM and concentrate under vacuum. Place the residue under vacuum for 2 hrs., to give 6,7-dichloro-1-tetrahydropyran-2-yl-3-(1-tetrahydropyran-2-ylpyrazol-4-yl)indazol-4-ol (150 mg, 43%) as a cream-colored solid. ES/MS (m/z): (³⁵Cl/³⁷Cl) 436.8/438.8 (M+H).

### Intermediate 620

### 2-[6,7-Dichloro-1-tetrahydropyran-2-yl-3-(1-tetrahydropyran-2-ylpyrazol-4-yl)indazol-4-yl]oxyacetonitrile

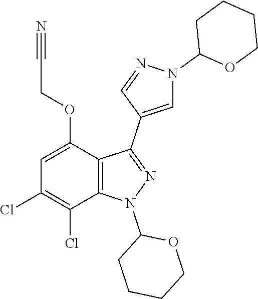 [SMILES: N#CCOc1cc(Cl)c(Cl)c2c1c(-c1cnn(C3CCCCO3)c1)nn2C1CCCCO1]

Combine 6,7-dichloro-1-tetrahydropyran-2-yl-3-(1-tetrahydropyran-2-ylpyrazol-4-yl)indazol-4-ol (150 mg, 0.333 mmol), cesium carbonate (39 mg, 0.12 mmol), and bromoacetonitrile (1.66 mL, 0.664 mmol, 0.400M in DMF) and stir at ambient temperature for 18 hrs. Add cesium carbonate (20 mg, 0.18 mmol) and bromoacetonitrile (0.83 mL, 0.33 mmol, 0.400M in DMF) and stir at ambient temperature for 6 hrs. Dilute with EtOAc, wash sequentially with saturated aqueous NH₄Cl, water, and saturated aqueous NaCl, dry over anhydrous sodium sulfate, filter and concentrate under vacuum. Purify by flash column chromatography eluting with EtOAc in hexanes. Dissolve the product in DCM and concentrate under vacuum. Place the residue under vacuum for 1 hr., to give 2-[6,7-dichloro-1-tetrahydropyran-2-yl-3-(1-tetrahydropyran-2-ylpyrazol-4-yl)indazol-4-yl]oxyacetonitrile (144 mg, 88%) as a yellow solid. ES/MS (m/z): (³⁵Cl/³⁷Cl) 476.2/478.2 (M+H).

### EXAMPLE 249

### 2-[[6,7-Dichloro-3-(1H-pyrazol-4-yl)-1H-indazol-4-yl]oxy]acetonitrile

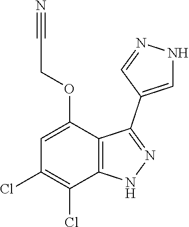 [SMILES: N#CCOc1cc(Cl)c(Cl)c2[nH]nc(-c3cn[nH]c3)c12]

Combine 2-[6,7-dichloro-1-tetrahydropyran-2-yl-3-(1-tetrahydropyran-2-ylpyrazol-4-yl)indazol-4-yl]oxyacetonitrile (144 mg, 0.293 mmol), DCM (1.5 mL), and TFA (0.22 mL, 2.9 mmol) and stir at ambient temperature for 4 hrs. Concentrate under a stream of N₂ and dilute the residue with EtOAc and saturated aqueous NaHCO₃. Separate the layers and extract the aqueous layer twice with EtOAc. Dry combined organic layers over anhydrous sodium sulfate, filter, and concentrate under vacuum. Purify by reverse phase chromatography, eluting with a 5-35% B in A gradient (A: 10 mM aqueous NH₄HCO₃ with 5% MeOH; B: ACN) to give 2-[[6,7-dichloro-3-(1H-pyrazol-4-yl)-1H-indazol-4-yl]oxy]acetonitrile (51 mg, 55%) as an off-white solid. ES/MS (m/z): (³⁵Cl/³⁷Cl) 307.8/309.8 (M+H). ¹H NMR (DMSO-d6): 13.73 (br s, 1H), 13.08 (br s, 1H), 8.10 (br s, 2H), 6.98 (s, 1H), 5.43 (s, 2H).

### Intermediate 621

### 6, 7-Dichloro-1-tetrahydropyran-2-yl-3-(1-tetrahydropyran-2-ylpyrazol-4-yl)indazol-4-amine

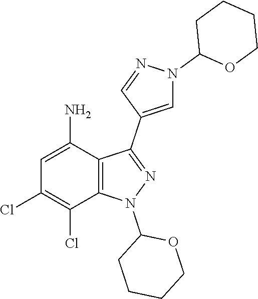 [SMILES: Nc1cc(Cl)c(Cl)c2c1c(-c1cnn(C3CCCCO3)c1)nn2C1CCCCO1]

Combine 4-bromo-6,7-dichloro-1-tetrahydropyran-2-yl-3-(1-tetrahydropyran-2-ylpyrazol-4-yl)indazole (761 mg, 1.52 mmol), tert-butyl carbamate (360 mg, 3.01 mmol), Cs₂CO₃ (1.10 g, 3.38 mmol), Pd₂(dba)₃ (₁28 mg, 0.136 mmol), XantPhos (162 mg, 0.272 mmol), and 1,4-dioxane (9.7 mL). Purge with N₂ and heat the mixture to 100° C. for 16 hrs. Cool to ambient temperature, filter through silica gel, rinse the silica gel with EtOAc, and concentrate the filtrate under vacuum. Purify by flash column chromatography eluting with EtOAc in hexanes. Dissolve the product in DCM and concentrate under vacuum. Dry under vacuum to give 6,7-dichloro-1-tetrahydropyran-2-yl-3-(1-tetrahydropyran-2-ylpyrazol-4-yl)indazol-4-amine (154 mg, 20%) as an orange solid. ES/MS (m/z): (³⁵Cl/³⁷Cl) 436.0/438.0 (M+H).

### EXAMPLE 250

### 3-[[6,7-Dichloro-3-(1H-pyrazol-4-yl)-1H-indazol-4-yl]amino]propan-1-ol

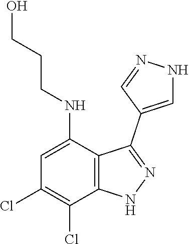 [SMILES: OCCCNc1cc(Cl)c(Cl)c2[nH]nc(-c3cn[nH]c3)c12]

Add sodium cyanoborohydride (58 mg, 0.89 mmol) to a solution of 6,7-dichloro-1-tetrahydropyran-2-yl-3-(1-tetrahydropyran-2-ylpyrazol-4-yl)indazol-4-amine (151 mg, 0.298 mmol), 3-[tert-butyl(dimethyl)silyl]oxypropanal (173 mg, 0.891 mmol), and acetic acid (17 μL, 13.4 mmol) in ethanol (7.4 mL) and stir at ambient temperature for 19 hrs. Add additional sodium cyanoborohydride (58 mg, 0.89 mmol) and stir at 60° C. for 3 hrs. Cool to ambient temperature and stir for 23 hrs. Add 3-[tert-butyl(dimethyl)silyl]oxypropanal (173 mg, 0.891 mmol) and stir at ambient temperature for 16 hrs. Add additional sodium cyanoborohydride (58 mg, 0.89 mmol) and stir at ambient temperature for 3 hrs. Add additional 3-[tert-butyl(dimethyl)silyl]oxypropanal (173 mg, 0.891 mmol) and stir at ambient temperature for 3 days. Add additional 3-[tert-butyl(dimethyl)silyl]oxypropanal (173 mg, 0.891 mmol) and stir at ambient temperature for 7 days. Concentrate under vacuum and purify by flash column chromatography eluting with EtOAc in hexanes to give impure N-[3-[tert-butyl(dimethyl)silyl]oxypropyl]-6,7-dichloro-1-tetrahydropyran-2-yl-3-(1-tetrahydropyran-2-ylpyrazol-4-yl)indazol-4-amine. Add MeOH (1.5 mL) and HCl (4.0 M) in dioxane (0.52 mL, 2.1 mmol) and stir at ambient temperature for 5 hrs. Concentrate under a stream of N₂ and purify by prep-HPLC to give 3-[[6,7-dichloro-3-(1H-pyrazol-4-yl)-1H-indazol-4-yl]amino]propan-1-ol (8 mg, 8%) as a white solid. ES/MS (m/z): (³⁵Cl/³⁷Cl) 325.8/327.8 (M+H). ¹H NMR (DMSO-d6): 13.37 (s, 1H), 13.18 (br s, 1H), 8.07 (br s, 1H), 7.78 (br s, 1H), 6.23 (s, 1H), 5.03 (t, J=5.1 Hz, 1H), 4.53 (t, J=5.0 Hz, 1H), 3.44 (q, J=5.7 Hz, 2H), 3.15 (q, J=6.2 Hz, 2H), 1.67 (quintet, J=6.4 Hz, 2H).

### Intermediate 622

### 9-Bromo-6,7-dichloro-2-ethyl-3,4-dihydropyrazino[1,2-a]indol-1-one

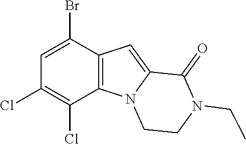 [SMILES: CCN1CCn2c(cc3c(Br)cc(Cl)c(Cl)c32)C1=O]

Cool a suspension of 9-bromo-6,7-dichloro-3,4-dihydro-2H-pyrazino[1,2-a]indol-1-one (2.00 g, 5.69 mmol) and DMF (28 mL) under N₂ to 0° C. and add NaH (455 mg, 11.4 mmol, 60% dispersion in mineral oil). Stir at 0° C. for 30 min and add iodoethane (0.69 mL, 8.5 mmol). Warm to ambient temperature and stir for 2 hrs. Quench with water and collect the precipitate by suction filtration. Wash with water (3×), diethyl ether, and hexanes to give 9-bromo-6,7-dichloro-2-ethyl-3,4-dihydropyrazino[1,2-a]indol-1-one (1.93 g, 90%) as an off-white solid. ES/MS (m/z): 360.8/362.8/364.8 (M+H).

### Intermediate 623

### tert-Butyl N-(6,7-dichloro-2-ethyl-1-oxo-3,4-dihydropyrazino[1,2-a]indol-9-yl)carbamate

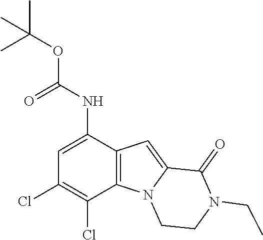 [SMILES: CCN1CCn2c(cc3c(NC(=O)OC(C)(C)C)cc(Cl)c(Cl)c32)C1=O]

Combine 9-bromo-6,7-dichloro-2-ethyl-3,4-dihydropyrazino[1,2-a]indol-1-one (500 mg, 1.37 mmol), tert-butyl carbamate (327 mg, 2.74 mmol), Cs₂CO₃ (1.01 g, 3.10 mmol), Pd₂(dba)₃ (₁17 mg, 0.124 mmol), XantPhos (148 mg, 0.248 mmol), and 1,4-dioxane (8.9 mL). Purge with N₂ and heat to 100° C. for 16 hrs., then cool to ambient temperature. Repeat the reaction using an identical procedure except for using 9-bromo-6,7-dichloro-2-ethyl-3,4-dihydropyrazino[1,2-a]indol-1-one (580 mg, 1.59 mmol), tert-butyl carbamate (379 mg, 3.17 mmol), Cs₂CO₃ (1.16 g, 3.56 mmol), Pd₂(dba)₃ (135 mg, 0.143 mmol), XantPhos (170 mg, 0.285 mmol), and 1,4-dioxane (10.2 mL) and then combine the crude products. Filter through silica gel, rinse the silica gel with EtOAc, and concentrate the filtrate under vacuum. Purify by flash column chromatography eluting with EtOAc in hexanes to give tert-butyl N-(6,7-dichloro-2-ethyl-1-oxo-3,4-dihydropyrazino[1,2-a]indol-9-yl)carbamate (1.03 g, 83%) as a light-yellow solid. ES/MS (m/z): (³⁵Cl/³⁷Cl) 398.0/400.0 (M+H).

### Intermediate 624

### tert-Butyl N-(10-bromo-6,7-dichloro-2-ethyl-1-oxo-3,4-dihydropyrazino[1,2-a]indol-9-yl)carbamate

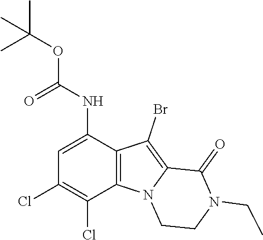 [SMILES: CCN1CCn2c(c(Br)c3c(NC(=O)OC(C)(C)C)cc(Cl)c(Cl)c32)C1=O]

Add NBS (552 mg, 3.07 mmol) to a solution of tert-butyl N-(6,7-dichloro-2-ethyl-1-oxo-3,4-dihydropyrazino[1,2-a]indol-9-yl)carbamate (1.03 g, 2.46 mmol) in DMF (25 mL) and stir at ambient temperature for 2 hrs. Dilute with water and EtOAc and add about 500 mg of Na₂SO₃. Mix thoroughly and separate the layers. Wash the organic layer sequentially with water and saturated aqueous NaCl, dry over anhydrous sodium sulfate, filter, and concentrate under vacuum. Purify by flash column chromatography eluting with DCM/EtOAc to give tert-butyl N-(10-bromo-6,7-dichloro-2-ethyl-1-oxo-3,4-dihydropyrazino[1,2-a]indol-9-yl)carbamate (1.00 g, 82%) as a light-orange solid. ES/MS (m/z): 476.0/478.0/480.0 (M+H).

### Intermediate 625

### tert-Butyl N-[6, 7-dichloro-2-ethyl-1-oxo-10-(1-tetrahydropyran-2-ylpyrazol-4-yl)-3,4-dihydropyrazino[1,2-a]indol-9-yl]carbamate

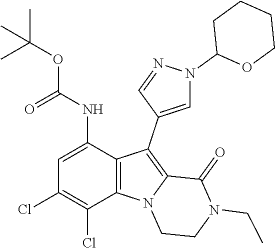 [SMILES: CCN1CCn2c(c(-c3cnn(C4CCCCO4)c3)c3c(NC(=O)OC(C)(C)C)cc(Cl)c(Cl)c32)C1=O]

Combine tert-butyl N-(10-bromo-6,7-dichloro-2-ethyl-1-oxo-3,4-dihydropyrazino[1,2-a]indol-9-yl)carbamate (972 mg, 1.96 mmol), 1-(tetrahydro-2H-pyran-2-yl)-4-(4,4,5,5-tetramethyl-1,3,2-dioxaborolan-2-yl)-1H-pyrazole (1.11 g, 3.91 mmol), sodium carbonate (622 mg, 5.86 mmol), Pd(dppf)Cl₂ (146 mg, 0.196 mmol), 1,4-dioxane (13.0 mL) and water (3.9 mL) under N₂. Heat the mixture to 90° C. for 3 hrs. Cool to ambient temperature and filter the solution through a plug of diatomaceous earth and silica gel and rinse the filter cake thoroughly with EtOAc. Concentrate the filtrate under vacuum. Purify by flash column chromatography eluting with EtOAc in DCM. Dissolve the product in DCM, concentrate under vacuum, and dry under vacuum at 60° C. Purify further by flash column chromatography eluting with MeOH in EtOAc. Dissolve the product in DCM, concentrate under vacuum, and dry under vacuum at 60° C. to give tert-butyl N-[6,7-dichloro-2-ethyl-1-oxo-10-(1-tetrahydropyran-2-ylpyrazol-4-yl)-3,4-dihydropyrazino[1,2-a]indol-9-yl]carbamate (824 mg, 74%) as a pale-yellow solid. ES/MS (m/z): (³⁵Cl/³⁷Cl) 548.2/550.2 (M+H).

### Intermediate 626

### 9-Amino-6,7-dichloro-2-ethyl-10-(1H-pyrazol-4-yl)-3,4-dihydropyrazino[1,2-a]indol-1-one dihydrochloride

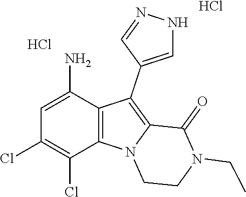 [SMILES: Cl ; CCN1CCn2c(c(-c3cn[nH]c3)c3c(N)cc(Cl)c(Cl)c32)C1=O ; Cl]

Combine tert-butyl N-[6,7-dichloro-2-ethyl-1-oxo-10-(1-tetrahydropyran-2-ylpyrazol-4-yl)-3,4-dihydropyrazino[1,2-a]indol-9-yl]carbamate (400 mg, 0.700 mmol), MeOH (3.5 mL) and HCl (4.0M) in dioxane (1.8 mL, 7.2 mmol) and stir at ambient temperature for 22 hrs. Concentrate under vacuum and dry under vacuum at 60° C. to give 9-amino-6,7-dichloro-2-ethyl-10-(1H-pyrazol-4-yl)-3,4-dihydropyrazino[1,2-a]indol-1-one dihydrochloride (335 mg, 99+%) as a light brown solid. ES/MS (m/z): (³⁵Cl/³⁷Cl) 364.0/366.0 (M+H).

### EXAMPLE 251

### N-[6,7-Dichloro-2-ethyl-1-oxo-10-(1H-pyrazol-4-yl)-3,4-dihydropyrazino[1,2-a]indol-9-yl]-2,2-difluoro-acetamide

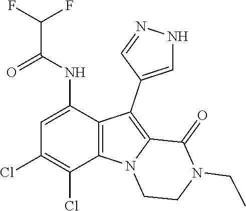 [SMILES: CCN1CCn2c(c(-c3cn[nH]c3)c3c(NC(=O)C(F)F)cc(Cl)c(Cl)c32)C1=O]

Cool a suspension of 9-amino-6,7-dichloro-2-ethyl-10-(1H-pyrazol-4-yl)-3,4-dihydropyrazino[1,2-a]indol-1-one dihydrochloride (111 mg, 0.232 mmol), DMAP (9 mg, 0.07 mmol), and DCM (2.3 mL) under N₂ to 0° C. and add pyridine (0.1 mL, 1 mmol) and difluoroacetic anhydride (61 μL, 0.47 mmol). Warm to ambient temperature and stir for 3 hrs. Add difluoroacetic anhydride (30 μL, 0.23 mmol) and stir at ambient temperature for 1 hr. Quench with MeOH (2 mL) and dilute with water and EtOAc. Adjust the pH to about 7 by the addition of saturated aqueous NaHCO₃ and separate the layers. Extract the aqueous layer again with EtOAc and combine the organic layers. Wash with saturated aqueous NaCl, dry over anhydrous sodium sulfate, filter, and concentrate under vacuum. Purify by flash column chromatography eluting with MeOH in EtOAc and then purify further by prep-HPLC. Dissolve the product in DCM and concentrate under vacuum. Dry under vacuum at 70° C. for 2 hrs., to give N-[6,7-dichloro-2-ethyl-1-oxo-10-(1H-pyrazol-4-yl)-3,4-dihydropyrazino[1,2-a]indol-9-yl]-2,2-difluoro-acetamide (68 mg, 66%) as a white solid. ES/MS (m/z): (³⁵Cl/³⁷Cl) 442.0/444.0 (M+H). ¹H NMR (DMSO-d6): 12.90 (s, 1H), 9.50 (s, 1H), 7.75 (s, 1H), 7.69 (s, 1H), 7.46 (s, 1H), 5.94 (t, J=53.7 Hz, 1H), 4.89 (dd, J=6.4, 5.0 Hz, 2H), 3.78 (dd, J=6.4, 5.0 Hz, 2H), 3.45 (q, J=7.1 Hz, 2H), 1.09 (t, J=7.1 Hz, 3H).

### Intermediate 627

### 9-Bromo-6,7-dichloro-2-(2-methoxyethyl)-3,4-dihydropyrazino[1,2-a]indol-1-one

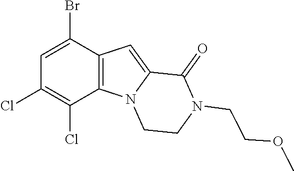 [SMILES: COCCN1CCn2c(cc3c(Br)cc(Cl)c(Cl)c32)C1=O]

Cool a suspension of 9-bromo-6,7-dichloro-3,4-dihydro-2H-pyrazino[1,2-a]indol-1-one (2.00 g, 5.69 mmol) and DMF (28 mL) under N₂ to 0° C. and add NaH (455 mg, 11.4 mmol, 60% dispersion in mineral oil). Stir at 0° C. for 30 min and add 2-bromomethyl ethyl ether (0.85 mL, 8.5 mmol). Warm to ambient temperature and stir for 22 hrs. Quench with water and collect the precipitate by suction filtration. Wash with water (3×), diethyl ether, and hexanes to give 9-bromo-6,7-dichloro-2-(2-methoxyethyl)-3,4-dihydropyrazino[1,2-a]indol-1-one (2.12 g, 91%) as a cream-colored solid. ES/MS (m/z): 391.0/393.0/395.0 (M+H).

### Intermediate 628

### tert-Butyl N-(6,7-dichloro-2-(2-methoxy ethyl)-1-oxo-3,4-dihydropyrazino[1,2-a]indol-9-yl)carbamate

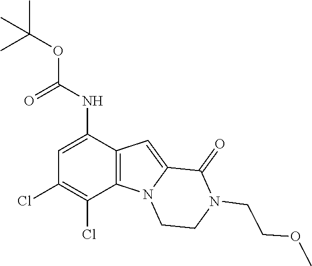 [SMILES: COCCN1CCn2c(cc3c(NC(=O)OC(C)(C)C)cc(Cl)c(Cl)c32)C1=O]

Combine 9-bromo-6,7-dichloro-2-(2-methoxyethyl)-3,4-dihydropyrazino[1,2-a]indol-1-one (508 mg, 1.37 mmol), tert-butyl carbamate (327 mg, 2.74 mmol), Cs₂CO₃ (1.01 g, 3.10 mmol), Pd₂(dba)₃ (₁17 mg, 0.124 mmol), XantPhos (148 mg, 0.248 mmol), and 1,4-dioxane (8.9 mL). Purge with N₂ and heat to 100° C. for 16 hrs., then cool to ambient temperature. Repeat the reaction using an identical procedure except for using 9-bromo-6,7-dichloro-2-(2-methoxyethyl)-3,4-dihydropyrazino[1,2-a]indol-1-one (720 mg, 1.76 mmol), tert-butyl carbamate (422 mg, 3.53 mmol), Cs₂CO₃ (1.29 g, 3.96 mmol), Pd₂(dba)₃ (₁50 mg, 0.159 mmol), XantPhos (189 mg, 0.317 mmol), and 1,4-dioxane (11.4 mL) and then combine the crude products. Filter through silica gel, rinse the silica gel with EtOAc, and concentrate the filtrate under vacuum. Purify by flash column chromatography eluting with EtOAc in hexanes. Purify further by flash column chromatography eluting with MeOH in DCM and again by flash column chromatography eluting with EtOAc in hexanes to give tert-butyl N-(6,7-dichloro-2-(2-methoxyethyl)-1-oxo-3,4-dihydropyrazino[1,2-a]indol-9-yl)carbamate (1.07 g, 82%) as an orange solid. ES/MS (m/z): (³⁵Cl/³⁷Cl) 428.0/430.0 (M+H).

### Intermediate 629

### tert-Butyl N-(10-bromo-6,7-dichloro-2-(2-methoxy ethyl)-1-oxo-3,4-dihydropyrazino[1,2-a]indol-9-yl)carbamate

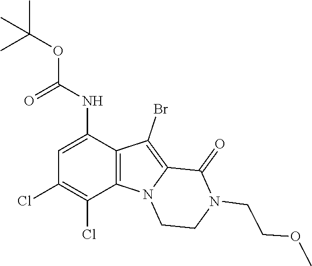 [SMILES: COCCN1CCn2c(c(Br)c3c(NC(=O)OC(C)(C)C)cc(Cl)c(Cl)c32)C1=O]

Add NBS (516 mg, 2.87 mmol) to a solution of tert-butyl N-(6,7-dichloro-2-(2-methoxyethyl)-1-oxo-3,4-dihydropyrazino[1,2-a]indol-9-yl)carbamate (1.07 g, 2.30 mmol) in DMF (23 mL) and stir at ambient temperature for 2 hrs. Dilute with water and EtOAc and add about 500 mg of Na₂SO₃. Mix thoroughly and separate the layers. Wash the organic layer sequentially with water and saturated aqueous NaCl, dry over anhydrous sodium sulfate, filter, and concentrate under vacuum. Purify by flash column chromatography eluting with EtOAc in DCM to give tert-butyl N-(10-bromo-6,7-dichloro-2-(2-methoxyethyl)-1-oxo-3,4-dihydropyrazino[1,2-a]indol-9-yl)carbamate (1.14 g, 87%) as a light-orange solid. ES/MS (m/z): 506.2/508.2/510.2 (M+H).

### Intermediate 630

tert-Butyl N-[6,7-dichloro-2-(2-methoxyethyl)-1-oxo-10-(1-tetrahydropyran-2-ylpyrazol-4-yl)-3,4-dihydropyrazino[1,2-a]indol-9-yl]carbamate

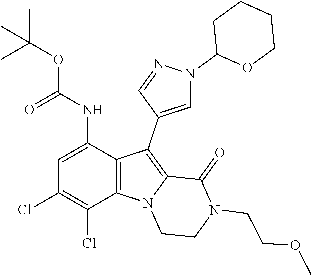 [SMILES: COCCN1CCn2c(c(-c3cnn(C4CCCCO4)c3)c3c(NC(=O)OC(C)(C)C)cc(Cl)c(Cl)c32)C1=O]

Combine tert-butyl N-(10-bromo-6,7-dichloro-2-(2-methoxyethyl)-1-oxo-3,4-dihydropyrazino[1,2-a]indol-9-yl)carbamate (1.14 g, 2.00 mmol), 1-(tetrahydro-2H-pyran-2-yl)-4-(4,4,5,5-tetramethyl-1,3,2-dioxaborolan-2-yl)-1H-pyrazole (1.14 g, 4.02 mmol), sodium carbonate (637 mg, 6.00 mmol), Pd(dppf)Cl₂ (149 mg, 0.200 mmol), 1,4-dioxane (13.3 mL) and water (4.0 mL) under N₂. Heat the mixture to 90° C. for 3 hrs. Cool to ambient temperature and filter the solution through a plug of diatomaceous earth and silica gel and rinse the filter cake thoroughly with EtOAc. Concentrate the filtrate under vacuum. Purify by flash column chromatography eluting with MeOH in EtOAc. Dissolve the product in DCM, concentrate under vacuum, and dry under vacuum at 60° C. to give tert-butyl N-[6,7-dichloro-2-(2-methoxyethyl)-1-oxo-10-(1-tetrahydropyran-2-ylpyrazol-4-yl)-3,4-dihydropyrazino[1,2-a]indol-9-yl]carbamate (907 mg, 74%) as a light orange solid. ES/MS (m/z): (³⁵Cl/³⁷Cl) 578.2/580.4 (M+H).

### Intermediate 631

### 9-Amino-6,7-dichloro-2-(2-methoxyethyl)-10-(1H-pyrazol-4-yl)-3,4-dihydropyrazino[1,2-a]indol-1-one dihydrochloride

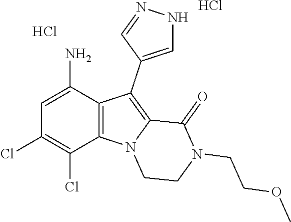 [SMILES: Cl ; COCCN1CCn2c(c(-c3cn[nH]c3)c3c(N)cc(Cl)c(Cl)c32)C1=O ; Cl]

Combine tert-butyl N-[6,7-dichloro-2-(2-methoxyethyl)-1-oxo-10-(1-tetrahydropyran-2-ylpyrazol-4-yl)-3,4-dihydropyrazino[1,2-a]indol-9-yl]carbamate (300 mg, 0.493 mmol), MeOH (2.5 mL) and HCl (4.0M) in dioxane (1.2 mL, 4.8 mmol) and stir at ambient temperature for 21 hrs. Concentrate under vacuum and dry under vacuum at 60° C. to give 9-amino-6,7-dichloro-2-(2-methoxyethyl)-10-(1H-pyrazol-4-yl)-3,4-dihydropyrazino[1,2-a]indol-1-one dihydrochloride (245 mg, 99+%) as a cream-colored solid. ES/MS (m/z): (³⁵Cl/³⁷Cl) 394.0/396.0 (M+H).

### EXAMPLE 252

### N-[6,7-Dichloro-2-(2-methoxyethyl)-1-oxo-10-(1H-pyrazol-4-yl)-3,4-dihydropyrazino[1,2-a]indol-9-yl]-2,2-difluoro-acetamide

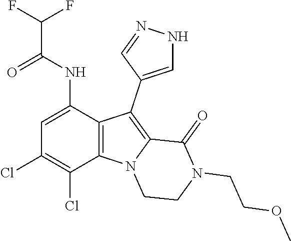 [SMILES: COCCN1CCn2c(c(-c3cn[nH]c3)c3c(NC(=O)C(F)F)cc(Cl)c(Cl)c32)C1=O]

Cool a suspension of 9-amino-6,7-dichloro-2-(2-methoxyethyl)-10-(1H-pyrazol-4-yl)-3,4-dihydropyrazino[1,2-a]indol-1-one dihydrochloride (115 mg, 0.231 mmol), DMAP (9 mg, 0.07 mmol), and DCM (2.3 mL) under N₂ to 0° C. and add pyridine (0.1 mL, 1 mmol) and difluoroacetic anhydride (60 μL, 0.46 mmol). Warm to ambient temperature and stir for 1 hr. Quench with MeOH (2 mL) and concentrate under vacuum. Dilute with water and EtOAc. Adjust the pH to about 7 by the addition of saturated aqueous NaHCO₃ and separate the layers. Extract the aqueous layer with EtOAc (5×) and combine the organic layers. Wash with saturated aqueous NaCl, dry over anhydrous sodium sulfate, filter, and concentrate under vacuum. Purify by flash column chromatography eluting with MeOH in DCM and dry under vacuum for 3 days to give N-[6,7-dichloro-2-(2-methoxyethyl)-1-oxo-10-(1H-pyrazol-4-yl)-3,4-dihydropyrazino[1,2-a]indol-9-yl]-2,2-difluoro-acetamide (94 mg, 83%) as an off-white solid. ES/MS (m/z): (³⁵Cl/³⁷Cl) 472.0/474.0 (M+H). ¹H NMR (DMSO-d6): 12.90 (s, 1H), 9.52 (s, 1H), 7.75 (s, 1H), 7.68 (s, 1H), 7.46 (s, 1H), 5.94 (t, J=53.7 Hz, 1H), 4.88 (dd, J=6.4, 5.0 Hz, 2H), 3.82 (dd, J=6.4, 5.0 Hz, 2H), 3.59 (t, J=5.5 Hz, 2H), 3.48 (t, J=5.5 Hz, 2H), 3.26 (s, 3H).

### Intermediate 632

### tert-Butyl 9-(tert-butoxycarbonylamino)-6,7-dichloro-10-iodo-3,4-dihydro-1H-pyrazino[1,2-a]indole-2-carboxylate

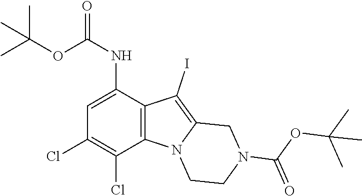 [SMILES: CC(C)(C)OC(=O)Nc1cc(Cl)c(Cl)c2c1c(I)c1n2CCN(C(=O)OC(C)(C)C)C1]

Suspend tert-butyl 9-(tert-butoxycarbonylamino)-6, 7-dichloro-3,4-dihydro-1H-pyrazino[1,2-a]indole-2-carboxylate (3.60 g, 6.31 mmol) and N-iodosuccinimide (2.20 g, 9.47 mmol) in DMF (50 mL). Stir at ambient temperature for 2 hrs. Pour onto iced water and filter. Dry the precipitate under vacuum for 16-18 hrs to give tert-butyl 9-(tert-butoxy carbonylamino)-6,7-dichloro-10-iodo-3,4-dihydro-1H-pyrazino[1,2-a]indole-2-carboxylate (3.60 g, 98%). ES/MS (m/z): (³⁵Cl/³⁷Cl) 580.2/582.2 (M−H).

### Intermediate 633

### tert-Butyl N-(2-acetyl-6,7-dichloro-3,4-dihydro-1H-pyrazino[1,2-a]indol-9-yl)carbamate

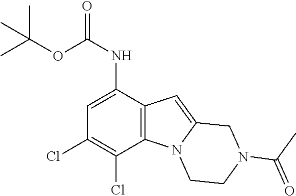 [SMILES: CC(=O)N1CCn2c(cc3c(NC(=O)OC(C)(C)C)cc(Cl)c(Cl)c32)C1]

Dilute together 1-(9-bromo-6,7-dichloro-3,4-dihydro-1H-pyrazino[1,2-a]indol-2-yl)ethanone (5.16 g, 14.3 mmol), tert-butyl carbamate (3.70 g., 31.4 mmol), tris(dibenzylideneacetone)dipalladium(0) (1.31 g., 1.43 mmol), 4,5-bis(diphenylphosphino)-9,9-dimethylxanthene (1.65 g, 2.85 mmol) and cesium carbonate (11.6 g, 35.6 mmol) in 1,4-dioxane (140 mL). Flush with N₂ for 10 min. Heat to 100° C. and stir under N₂ for 16-18 hrs. Filter through diatomaceous earth and concentrate under vacuum. Purify by flash column chromatography eluting with hexane in MeOH and DCM to give tert-butyl N-(2-acetyl-6,7-dichloro-3,4-dihydro-1H-pyrazino[1,2-a]indol-9-yl)carbamate (5.3 g, 93%). ES/MS (m/z): (³⁵Cl/³⁷Cl) 396.2/398.2 (M−H).

### Intermediate 634

### tert-Butyl N-(2-acetyl-6,7-dichloro-10-iodo-3,4-dihydro-1H-pyrazino[1,2-a]indol-9-yl)carbamate

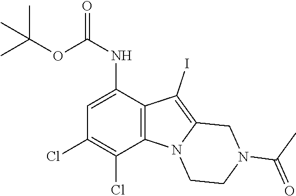 [SMILES: CC(=O)N1CCn2c(c(I)c3c(NC(=O)OC(C)(C)C)cc(Cl)c(Cl)c32)C1]

Dilute tert-butyl N-(2-acetyl-6, 7-dichloro-3,4-dihydro-1H-pyrazino[1,2-a]indol -9-yl)carbamate (5.30 g, 13.3 mmol) and N-iodosuccinimide (4.50 g, 20.0 mmol) with DMF (50 mL). Stir at ambient temperature for 2 hrs. Pour onto ice water and filter. Vacuum dry precipitate for 16-18 hrs to give tert-butyl N-(2-acetyl-6,7-dichloro-10-iodo-3,4-dihydro-1H-pyrazino[1,2-a]indol-9-yl)carbamate (6.28 g, 90%). ES/MS (m/z): (³⁵Cl/³⁷Cl) 522.0/524.0 (M+H).

### Intermediate 635

### tert-Butyl N-[2-acetyl-6,7-dichloro-10-(1-tetrahydropyran-2-ylpyrazol-4-yl)-3,4-dihydro-1H-pyrazino[1,2-a]indol-9-yl]carbamate

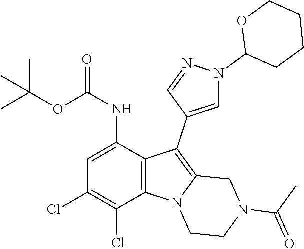 [SMILES: CC(=O)N1CCn2c(c(-c3cnn(C4CCCCO4)c3)c3c(NC(=O)OC(C)(C)C)cc(Cl)c(Cl)c32)C1]

Dilute tert-butyl N-(2-acetyl-6,7-dichloro-10-iodo-3,4-dihydro-1H-pyrazino[1,2-a]indol-9-yl)carbamate (4.50 g, 8.59 mmol) and 1-(tetrahydro-2h-pyran-2-yl)-4-(4,4,5,5-tetramethyl-1,3,2-dioxaborolan-2-yl)-1h-pyrazole (3.02 g, 10.30 mmol), Pd(dtbpf)Cl₂ (715 mg, 0.859 mmol), and sodium carbonate (1.82 g, 17.2 mmol) with 1,4-dioxane (100 mL) and water (20 mL). Flush with N₂ for 10 min. Heat at 90° C. for 1.5 hrs. Cool to ambient temperature, filter through diatomaceous earth, and concentrate under vacuum.

Dissolve in EtOAc, dry over sodium sulfate, filter, concentrate under vacuum and purify by flash column chromatography eluting with EtOAc in hexanes to give tert-butyl N-[2-acetyl-6,7-dichloro-10-(1-tetrahydropyran-2-ylpyrazol-4-yl)-3,4-dihydro-1H-pyrazino[1,2-a]indol-9-yl]carbamate (4.2 g, 89%). ES/MS (m/z): (³⁵Cl/³⁷Cl) 546.0/548.0 (M−H).

### Intermediate 636

### tert-Butyl N-[2-acetyl -6, 7-dichloro-10-(1-tetrahydropyran-2-ylpyrazol-4-yl)-3,4-dihydro-1H-pyrazino[1,2-a]indol-9-yl]-N-[3-[tert-butyl(dimethyl)silyl]oxypropyl]carbamate

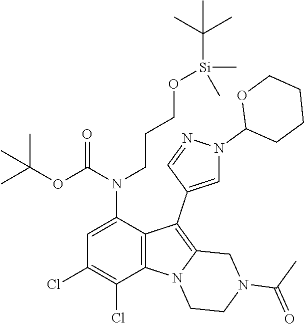 [SMILES: CC(=O)N1CCn2c(c(-c3cnn(C4CCCCO4)c3)c3c(N(CCCO[Si](C)(C)C(C)(C)C)C(=O)OC(C)(C)C)cc(Cl)c(Cl)c32)C1]

Dilute tert-butyl N-[2-acetyl-6,7-dichloro-10-(1-tetrahydropyran-2-ylpyrazol-4-yl)-3,4-dihydro-1H-pyrazino[1,2-a]indol-9-yl]carbamate (300 mg, 0.547 mmol) with DMF (10 mL). Add sodium hydride (90 mg, 2.19 mmol, 60% in mineral oil) and stir under N₂ for 10 min. Add tert-butyl(3-iodopropoxy)dimethylsilane (0.670 g, 2.19 mmol) and stir mixture under N₂ for 2 hrs. Quench with ice water. Extract with EtOAc, dry over sodium sulfate and concentrate under vacuum to give tert-butyl N-[2-acetyl-6,7-dichloro-10-(1-tetrahydropyran-2-ylpyrazol-4-yl)-3,4-dihydro-1H-pyrazino[1,2-a]indol-9-yl]-N-[3-[tert-butyl(dimethyl)silyl]oxypropyl]carbamate (0.37 g, 90%). ES/MS (m/z): (³⁵Cl/³⁷Cl) 718.4/720.4 (M+H).

### EXAMPLE 253

### 1-[6,7-Dichloro-9-(3-hydroxypropylamino)-10-(1H-pyrazol-4-yl)-3,4-dihydro-1H-pyrazino[1,2-a]indol-2-yl]ethanone

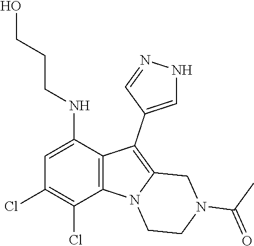 [SMILES: CC(=O)N1CCn2c(c(-c3cn[nH]c3)c3c(NCCCO)cc(Cl)c(Cl)c32)C1]

Dilute tert-butyl N-[2-acetyl-6,7-dichloro-10-(1-tetrahydropyran-2-ylpyrazol-4-yl)-3,4-dihydro-1H-pyrazino[1,2-a]indol-9-yl]-N-[3-[tert-butyl(dimethyl)silyl]oxypropyl]carbamate (0.37 g, 0.51 mmol) with DCM (5.1 mL) and stir at 0° C. for 10 min. Add TFA (5.1 mL, 68 mmol) dropwise and stir at ambient temperature for 2 hrs. Concentrate under vacuum and purify by removing the solvent through rotary evaporator. Purify residual oil by HPLC to give 1-[6,7-dichloro-9-(3-hydroxypropylamino)-10-(1H-pyrazol-4-yl)-3,4-dihydro-1H-pyrazino[1,2-a]indol-2-yl]ethanone (128 mg, 59%). ES/MS (m/z): (³⁵Cl/³⁷Cl) 422.2/424.2 (M+H).

### Intermediate 637

### tert-Butyl N-[2-acetyl-6,7-dichloro-10-(1-tetrahydropyran-2-ylpyrazol-4-yl)-3,4-dihydro-1H-pyrazino[1,2-a]indol-9-yl]-N-(cyanomethyl)carbamate

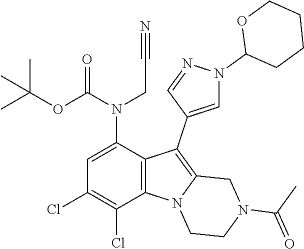 [SMILES: CC(=O)N1CCn2c(c(-c3cnn(C4CCCCO4)c3)c3c(N(CC#N)C(=O)OC(C)(C)C)cc(Cl)c(Cl)c32)C1]

Dilute tert-butyl N-[2-acetyl-6,7-dichloro-10-(1-tetrahydropyran-2-ylpyrazol-4-yl)-3,4-dihydro-1H-pyrazino[1,2-a]indol-9-yl]carbamate (200 mg, 0.365 mmol) with DMF (10 mL) and add sodium hydride (58 mg, 1.46 mmol, 60% in mineral oil). Add chloroacetonitrile (95 μL, 1.46 mmol) and stir at ambient temperature for 2 hrs. Quench with ice water and extract with EtOAc. Dry over sodium sulfate and concentrate under vacuum to give tert-butyl N-[2-acetyl-6,7-dichloro-10-(1-tetrahydropyran-2-ylpyrazol-4-yl)-3,4-dihydro-1H-pyrazino[1,2-a]indol-9-yl]-N-(cyanomethyl)carbamate (0.2 g, 90%). ES/MS (m/z): (³⁵Cl/³⁷Cl) 587.3/589.3 (M+H).

### EXAMPLE 254

### 2-[[2-Acetyl-6,7-dichloro-10-(1H-pyrazol-4-yl)-3,4-dihydro-1H-pyrazino[1,2-a]indol-9-yl]amino]acetonitrile

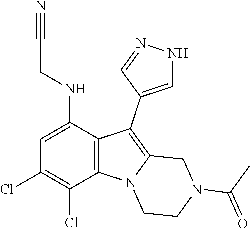 [SMILES: CC(=O)N1CCn2c(c(-c3cn[nH]c3)c3c(NCC#N)cc(Cl)c(Cl)c32)C1]

Dilute tert-butyl N-[2-acetyl-6,7-dichloro-10-(1-tetrahydropyran-2-ylpyrazol-4-yl)-3,4-dihydro-1H-pyrazino[1,2-a]indol-9-yl]-N-(cyanomethyl)carbamate (0.20 g, 0.30 mmol) in DCM (3.0 mL) and cool to 0° C. Add TFA (3.0 mL, 50 mmol) at 0° C. and warm to ambient temperature for over 4 hrs. Concentrate under vacuum and purify by HPLC to give 2-[[2-acetyl-6,7-dichloro-10-(1H-pyrazol-4-yl)-3,4-dihydro-1H-pyrazino[1,2-a]indol-9-yl]amino]acetonitrile (31 mg, 20%). ES/MS (m/z): (³⁵Cl/³⁷Cl) 403.0/405.0 (M+H). ¹H NMR (DMSO-d6): 13.11 (s, 1H), 7.83-7.81 (m, 1H), 7.53-7.51 (m, 2H), 6.57 (s, 1H), 5.02-5.99 (m, 1H), 4.73-4.59 (m, 3H), 4.31 (d, 2H), 3.94-3.89 (m, 2H), 2.11 (S, 3H).

### Intermediate 638

### tert-Butyl N-[2-acetyl -6, 7-dichloro-10-(1-tetrahydropyran-2-ylpyrazol-4-yl)-3,4-dihydro-1H-pyrazino[1,2-a]indol-9-yl]-N-ethyl-carbamate

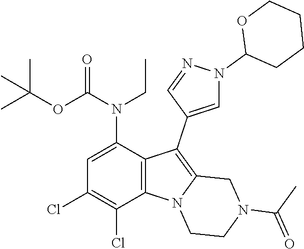 [SMILES: CCN(C(=O)OC(C)(C)C)c1cc(Cl)c(Cl)c2c1c(-c1cnn(C3CCCCO3)c1)c1n2CCN(C(C)=O)C1]

Dilute tert-butyl N-[2-acetyl-6,7-dichloro-10-(1-tetrahydropyran-2-ylpyrazol-4-yl)-3,4-dihydro-1H-pyrazino[1,2-a]indol-9-yl]carbamate (200 mg, 0.365 mmol) in DMF (10 mL) and add sodium hydride (58.0 mg, 1.46 mmol, 60% in mineral oil). Stir at 0° C. under N₂ for 10 min. Add iodoethane (117 μL, 1.46 mmol) and stir at ambient temperature for 2 hrs. Quench with ice water, extract with EtOAc, dry over sodium sulfate and concentrate under vacuum to give tert-butyl N-[2-acetyl-6,7-dichloro-10-(1-tetrahydropyran-2-ylpyrazol-4-yl)-3,4-dihydro-1H-pyrazino[1,2-a]indol-9-yl]-N-ethyl-carbamate (0.2 g, 99+%). ES/MS (m/z): (³⁵Cl/³⁷Cl) 576.0/578.0 (M+H).

### EXAMPLE 255

### 1-(6,7-dichloro-9-(ethylamino)-10-(1H-pyrazol-4-yl)-3,4-dihydropyrazino[1,2-a]indol -2(1H)-yl)ethan-1-one hydrochloride

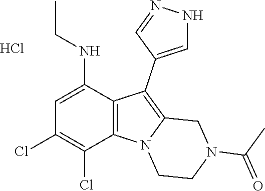 [SMILES: Cl ; CCNc1cc(Cl)c(Cl)c2c1c(-c1cn[nH]c1)c1n2CCN(C(C)=O)C1]

Suspend tert-butyl N-[2-acetyl-6,7-dichloro-10-(1-tetrahydropyran-2-ylpyrazol-4-yl)-3,4-dihydro-1H-pyrazino[1,2-a]indol-9-yl]-N-ethyl-carbamate (0.2 g, 0.3 mmol) in HCl (3.0 mL, 10 mmol, 4M in 1,4-dioxane) at 0° C. Warm to ambient temperature and stir for 2 hrs. Concentrate under vacuum and purify by HPLC to give 1-(6,7-dichloro-9-(ethylamino)-10-(1H-pyrazol-4-yl)-3,4-dihydropyrazino[1,2-a]indol-2(1H)-yl)ethan-1-one hydrochloride (20 mg, 10%). ES/MS (m/z): (³⁵Cl/³⁷Cl) 392.0/394.0 (M+H).

### Intermediate 639

### 1-(9-amino-6,7-dichloro-10-(1H-pyrazol-4-yl)-3,4-dihydropyrazino[1,2-a]indol-2(1H)-yl)ethan-1-one 2,2,2-trifluoroacetate

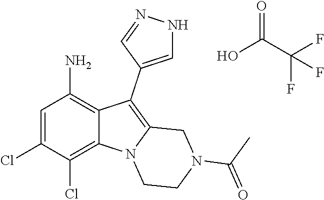 [SMILES: CC(=O)N1CCn2c(c(-c3cn[nH]c3)c3c(N)cc(Cl)c(Cl)c32)C1 ; O=C(O)C(F)(F)F]

Dissolve tert-butyl N-[2-acetyl-6,7-dichloro-10-(1-tetrahydropyran-2-ylpyrazol-4-yl)-3,4-dihydro-1H-pyrazino[1,2-a]indol-9-yl]carbamate (200 mg, 0.365 mmol) in DCM (4 mL) and stir at 0° C. for 10 min. Add TFA (4.0 mL, 48.22 mmol) dropwise. Warm to ambient temperature and stir for 2 hrs. Concentrate under vacuum to give 1-(9-amino-6,7-dichloro-10-(1H-pyrazol-4-yl)-3,4-dihydropyrazino[1,2-a]indol-2(1H)-yl)ethan-1-one 2,2,2-trifluoroacetate (0.172 g, 100%). ES/MS (m/z): (³⁵Cl/³⁷Cl) 364.0/366.0 (M+H).

### EXAMPLE 256

### N-[2-Acetyl-6,7-dichloro-10-(1H-pyrazol-4-yl)-3,4-dihydro-1H-pyrazino[1,2-a]indol-9-yl]-2,2,2-trifluoro-acetamide

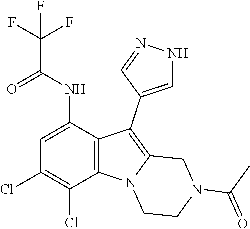 [SMILES: CC(=O)N1CCn2c(c(-c3cn[nH]c3)c3c(NC(=O)C(F)(F)F)cc(Cl)c(Cl)c32)C1]

Suspend 1-(9-amino-6,7-dichloro-10-(1H-pyrazol-4-yl)-3,4-dihydropyrazino[1,2-a]indol-2(1H)-yl)ethan-1-one 2,2,2-trifluoroacetate (0.172 g, 0.360 mmol) in DCM (4 mL) and stir at 0° C. for 10 minutes. Add pyridine (300 μL, 3.65 mmol). Dropwise, add TFAA (160 μL, 1.09 mmol) stir at 0° C. for 10 min and ambient temperature for 10 min. Pour reaction into DCM (5 mL) and saturated aqueous NaHCO₃ stirring vigorously. Separate the organic layer, dry over sodium sulfate, and concentrate under vacuum. Purify by HPLC to give N-[2-acetyl-6,7-dichloro-10-(1H-pyrazol-4-yl)-3,4-dihydro-1H-pyrazino[1,2-a]indol-9-yl]-2,2,2-trifluoro-acetamide (103 mg, 62.2%). ES/MS (m/z): (³⁵Cl/³⁷Cl) 460.0/462.0 (M+H). ¹H NMR (DMSO-d6): 12.94 (s, 1H), 10.74 (s, 1H), 7.69 (s, 1H), 7.49 (s, 1H), 7.29 (s, 1H), 4.98-4.64 (m, 4H), 3.98-3.92 (m, 2H), 2.12 (s, 3H).

### Intermediate 640

### [2-[[5-[6,7-Dichloro-3-(1H-pyrazol-4-yl)-1H-indol-2-yl]-1,3,4-oxadiazol-2-yl]amino]-2-oxo-ethyl] acetate

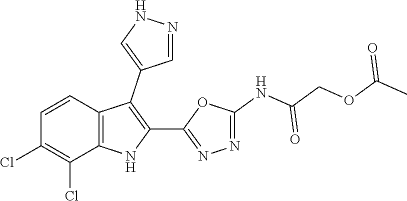 [SMILES: CC(=O)OCC(=O)Nc1nnc(-c2[nH]c3c(Cl)c(Cl)ccc3c2-c2cn[nH]c2)o1]

Dissolve 5-[6,7-dichloro-3-(1H-pyrazol-4-yl)-1H-indol-2-yl]-1,3,4-oxadiazol-2-amine (100 mg, 0.2403 mmol) in pyridine (10 mL, 124 mmol) and add (2-chloro-2-oxo-ethyl) acetate (95 mg, 0.721 mmol). Stir at ambient temperature for 48 hrs. Stir for 1 hr. at reflux temperature. Cool to ambient temperature, pour onto ice water. Extract with EtOAc, wash with dilute HCl, saturated aqueous NaCl and dry over sodium sulfate. Concentrate under vacuum to give [2-[[5-[6,7-dichloro-3-(1H-pyrazol-4-yl)-1H-indol-2-yl]-1,3,4-oxadiazol-2-yl]amino]-2-oxo-ethyl] acetate (100 mg, 99+%). ES/MS (m/z): (³⁵Cl/³⁷Cl) 435.0/437.0 (M+H).

### EXAMPLE 257

### N-[5-[6,7-Dichloro-3-(1H-pyrazol-4-yl)-1H-indol-2-yl]-1,3,4-oxadiazol-2-yl]-2-hydroxy-acetamide

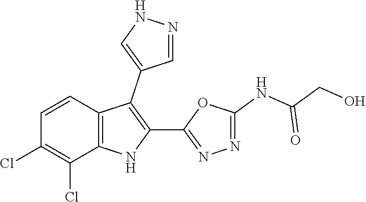 [SMILES: O=C(CO)Nc1nnc(-c2[nH]c3c(Cl)c(Cl)ccc3c2-c2cn[nH]c2)o1]

Dilute [2-[[5-[6,7-dichloro-3-(1H-pyrazol-4-yl)-1H-indol-2-yl]-1,3,4-oxadiazol -2-yl]amino]-2-oxo-ethyl] acetate (100 mg, 0.230 mmol) and potassium carbonate (65 mg, 0.46 mmol) with MeOH (5 mL). Heat at 80° C. for 1 hr. Concentrate under vacuum and dilute with water. Filter and purify precipitate by HPLC to give N-[5-[6,7-dichloro-3-(1H-pyrazol-4-yl)-1H-indol-2-yl]-1,3,4-oxadiazol-2-yl]-2-hydroxy-acetamide (67 mg, 74%). ES/MS (m/z): (³⁵Cl/³⁷Cl) 393.0/395.0 (M+H).

### Intermediate 641

### 6,7-Dichloro-3-(1-(tetrahydro-2H-pyran-2-yl)-1H-pyrazol-4-yl)-1H-indole-2-carboxylic acid

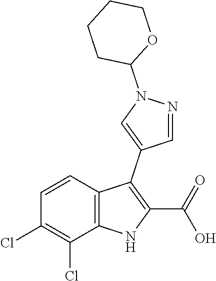 [SMILES: O=C(O)c1[nH]c2c(Cl)c(Cl)ccc2c1-c1cnn(C2CCCCO2)c1]

Dissolve lithium hydroxide (0.500 g, 19.60 mmol) in water (20 mL). Add a solution of ethyl 6,7-dichloro-3-[1-[(2R)-tetrahydropyran-2-yl]pyrazol-4-yl]-1H-indole-2-carboxylate (800 mg, 1.96 mmol) in EtOH (20 mL). Stir at ambient temperature for 16-18 hrs. Remove EtOH under vacuum and extract the residual aqueous solution with EtOAc (3×). Dry combined EtOAc, wash with saturated aqueous NaCl and dry over sodium sulfate. Concentrate under vacuum to give 6,7-dichloro-3-(1-(tetrahydro-2H-pyran-2-yl)-1H-pyrazol-4-yl)-1H-indole-2-carboxylic acid (0.60 g, 81%). ES/MS (m/z): (³⁵Cl/³⁷Cl) 380.0/382.0 (M+H).

### Intermediate 642

### tert-Butyl (6, 7-dichloro-3-(1-(tetrahydro-2H-pyran-2-yl)-1H-pyrazol-4-yl)-1H-indole-2-carbonyl)-L-prolinate

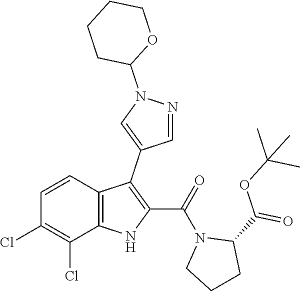 [SMILES: CC(C)(C)OC(=O)[C@@H]1CCCN1C(=O)c1[nH]c2c(Cl)c(Cl)ccc2c1-c1cnn(C2CCCCO2)c1]

Suspend 6,7-dichloro-3-(1-(tetrahydro-2H-pyran-2-yl)-1H-pyrazol-4-yl)-1H-indole-2-carboxylic acid (100 mg, 0.263 mmol), tert-butyl (2S)-pyrrolidine-2-carboxylate (70 mg, 0.395 mmol), HATU (0.200 g, 0.526 mmol) and DIPEA (0.200 mL, 1.05 mmol) in DMF (1.5 mL). Stir for 1 hr. at ambient temperature. Quench with saturated aqueous NaHCO₃, extract with EtOAc (3×) and wash combined EtOAc with saturated aqueous NH₄Cl. Dry over sodium sulfate and concentrate to give tert-butyl (6,7-dichloro-3-(1-(tetrahydro-2H-pyran-2-yl)-1H-pyrazol-4-yl)-1H-indole-2-carbonyl)-L-prolinate (135 mg, 99+%). ES/MS (m/z): (³⁵Cl/³⁷Cl) 531.5/533.6 (M−H).

### EXAMPLE 258

### (2 S)-1-[6,7-Dichloro-3-(1H-pyrazol-4-yl)-1H-indole-2-carbonyl]pyrrolidine-2-carb oxylic acid

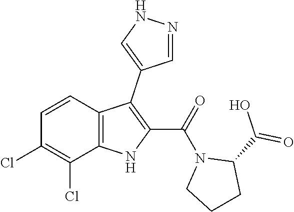 [SMILES: O=C(O)[C@@H]1CCCN1C(=O)c1[nH]c2c(Cl)c(Cl)ccc2c1-c1cn[nH]c1]

Cool HCl (2.0 mL, 8.0 mmol, 4M in 1,4-dioxane) to 0° C. Add tert-butyl (6,7-dichloro-3-(1-(tetrahydro-2H-pyran-2-yl)-1H-pyrazol-4-yl)-1H-indole-2-carbonyl)-L-prolinate (135 mg, 0.253 mmol). Stir at ambient temperature for 3 hrs. Concentrate under vacuum and purify by HPLC to give (2S)-1-[6,7-dichloro-3-(1H-pyrazol-4-yl)-1H-indole-2-carbonyl]pyrrolidine-2-carboxylic acid (40 mg, 37%). ES/MS (m/z): (³⁵Cl/³⁷Cl) 393.0/395.0 (M+H).

### EXAMPLES 259 & 260

### 5-[6,7-Dichloro-10-(1H-pyrazol-4-yl)-3,4-dihydro-1H-pyrazino[1,2-a]indole-2-carbonyl]oxazolidin-2-one

### Isomer 1 and Isomer 2

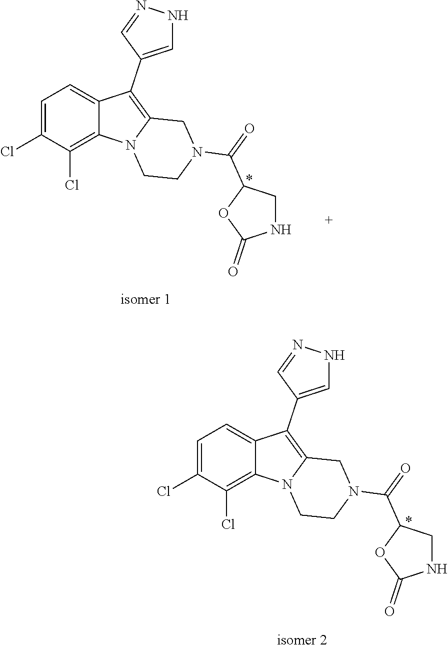 [SMILES: O=C1NCC(C(=O)N2CCn3c(c(-c4cn[nH]c4)c4ccc(Cl)c(Cl)c43)C2)O1 ; O=C1NCC(C(=O)N2CCn3c(c(-c4cn[nH]c4)c4ccc(Cl)c(Cl)c43)C2)O1]

Purify 5-[6,7-dichloro-10-(1H-pyrazol-4-yl)-3,4-dihydro-1H-pyrazino[1,2-a]indole-2-carbonyl]oxazolidin-2-one (32 mg, 0.076 mmol) by chiral supercritical fluid chromatography to give the titled compounds (Isomer 1-13.4 mg, 42%; Isomer 2-11.6 mg, 36%). Retention time=1.50 min (>99% ee) and retention time=3.00 min (96% ee). Column: Chiralpak AD-H, 21×250 mm; Mobile Phase: 40% IPA: 60% CO₂; Flow Rate: 80 mL/min; Column temperature: 40° C.; Detection: 225 nM. ES/MS (m/z): (³⁵Cl/³⁷Cl) 419.8/421.8 (M+H). ¹H NMR (DMSO-d6): 13.15-13.12 (m, 1H), 8.08-8.05 (m, 1H), 7.82-7.72 (m, 2H), 7.67-7.63 (m, 1H), 7.29 (d, J=8.5 Hz, 1H), 5.65-5.62 (m, 1H), 5.06-4.95 (m, 2H), 4.80-4.73 (m, 2H), 4.06-4.01 (m, 2H), 3.69-3.61 (m, 2H).

### EXAMPLES 261 & 262

### 3-[[6,7-Dichloro-3-(1H-pyrazol-4-yl)-1H-indol-4-yl]amino]propane-1,2-diol (Isomer 1 and Isomer 2)

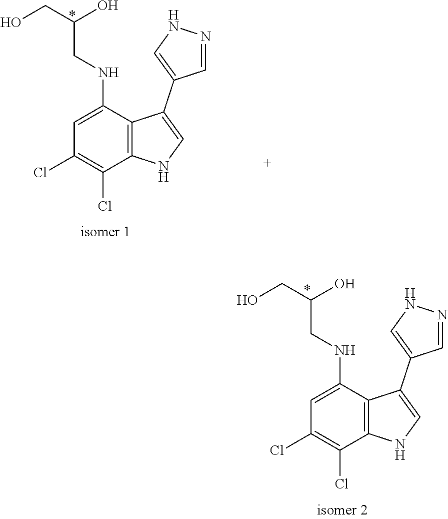 [SMILES: OCC(O)CNc1cc(Cl)c(Cl)c2[nH]cc(-c3cn[nH]c3)c12 ; OCC(O)CNc1cc(Cl)c(Cl)c2[nH]cc(-c3cn[nH]c3)c12]

Purify 3-[[6,7-dichloro-3-(1H-pyrazol-4-yl)-1H-indol-4-yl]amino]propane-1,2-diol (45.2 mg, 0.132 mmol) by supercritical fluid chromatography to give the titled compounds (Isomer 1-14.2 mg, 31%; Isomer 2-17.8 mg, 39%). Retention time (isomer 1)=1.39 min (>99% ee) and Retention time (isomer 2)=1.97 min (97% ee). Column: Chiralcel OJ-H, 21×250 mm; Mobile Phase: 30% EtOH: 70% CO₂; Flow Rate: 80 mL/min; Column temperature: 40° C.; Detection: 225 nM. ES/MS (m/z): (³⁵Cl/³⁷Cl) 340.8/342.8 (M+H). ¹H NMR (DMSO-d6): 12.98-12.96 (brs, 1H), 11.41 (s, 1H), 7.83 (s, 1H), 7.60 (s, 1H), 7.13 (d, J=2.5 Hz, 1H), 6.23 (s, 1H), 4.97-4.93 (m, 1H), 4.78 (d, J=5.1 Hz, 1H), 4.62 (t, J=5.6 Hz, 1H), 3.64-3.62 (m, 1H), 3.30-3.23 (m, 3H), 2.95-2.89 (m, 1H).

### EXAMPLE 263

### N-[2-Acetyl-6,7-dichloro-10-(1H-pyrazol-4-yl)-3,4-dihydro-1H-pyrazino[1,2-a]indol-9-yl]-2,2-difluoro-acetamide

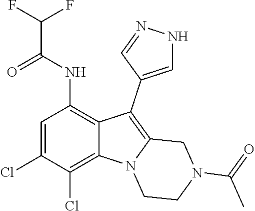 [SMILES: CC(=O)N1CCn2c(c(-c3cn[nH]c3)c3c(NC(=O)C(F)F)cc(Cl)c(Cl)c32)C1]

Dilute tert-butyl N-[2-acetyl-6,7-dichloro-10-(1-tetrahydropyran-2-ylpyrazol-4-yl)-3,4-dihydro-1H-pyrazino[1,2-a]indol-9-yl]carbamate (115 mg, 0.210 mmol) with HCl (2.0 mL, 8.0 mmol, 4M in dioxane) and stir for 1 hr., at ambient temperature. Concentrate under vacuum. Dilute in DCM (2.0 mL), add DMAP (8.9 mg, 0.071 mmol) and cool to 0° C. Add pyridine (0.10 mL, 1.0 mmol) and difluoroacetic anhydride (0.05 mL, 0.4 mmol). Stir for 1 hr., concentrate under vacuum and purify by reverse phase chromatography to give N-[2-acetyl-6,7-dichloro-10-(1H-pyrazol-4-yl)-3,4-dihydro-1H-pyrazino[1,2-a]indol-9-yl]-2,2-difluoro-acetamide (47.5 mg, 59%). ES/MS (m/z): (³⁵Cl/³⁷Cl) 442.0/444.0 (M+H). ¹H NMR (DMSO-d6): 13.10-13.08 (brs, 1H), 9.76-9.74 (m, 1H), 7.86-7.84 (m, 1H), 7.50 (s, 2H), 6.10-5.83 (m, 1H), 4.79-4.72 (m, 2H), 4.69-4.66 (m, 2H), 3.98-3.91 (m, 2H), 2.12 (s, 3H).

### EXAMPLES 264 & 265

### 2-(4-acetylmorpholin-2-yl)-1-(6,7-dichloro-10-(1H-pyrazol-4-yl)-3,4-dihydropyrazino[1,2-a]indol-2(1H)-yl)ethan-1-one

### Isomer 1 and Isomer 2

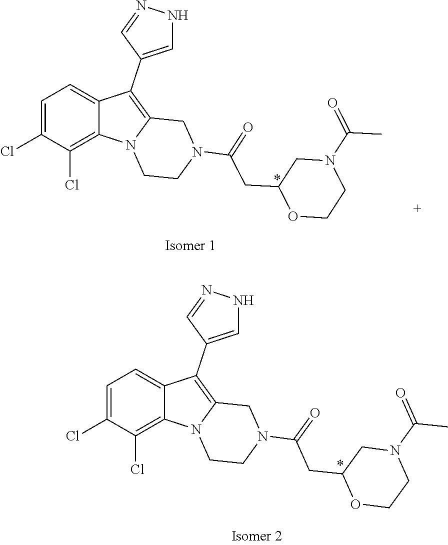 [SMILES: CC(=O)N1CCOC(CC(=O)N2CCn3c(c(-c4cn[nH]c4)c4ccc(Cl)c(Cl)c43)C2)C1 ; CC(=O)N1CCOC(CC(=O)N2CCn3c(c(-c4cn[nH]c4)c4ccc(Cl)c(Cl)c43)C2)C1]

Purify 2-(4-acetylmorpholin-2-yl)-1-(6,7-dichloro-10-(1H-pyrazol-4-yl)-3,4-dihydropyrazino[1,2-a]indol-2(1H)-yl)ethan-1-one (70 mg, 0.15 mmol) by supercritical fluid chromatography to give the crude titled compounds (Isomer 1-24.4 mg; Isomer 2-25.7 mg). Retention time (isomer 1)=3.56 min (>99% ee) and retention time (isomer 2)=4.61 min (97% ee). Column: Chiralpak IG (Amy-3), 30×250 mm; Mobile Phase: 50% EtOH: 50% CO₂; Flow Rate: 100 mL/min; Column temperature: 40° C.; Detection: 240 nM. Purify the separated isomers by reverse phase column chromatography to give the titled compounds (Isomer 1-8.7 mg, 0.018 mmol, 12%; Isomer 2-11.3 mg, 0.023 mmol, 16%). ES/MS (m/z): (³⁵Cl/³⁷Cl) 476.0/478.0 (M+H). ¹H NMR (DMSO-d6): 13.33-13.29 (m, 1H), 7.95-7.89 (m, 2H), 7.67-7.63 (m, 1H), 7.28 (d, J=8.5 Hz, 1H), 4.99-4.92 (m, 2H), 4.76-4.67 (m, 2H), 4.35-4.12 (m, 1H), 4.04-3.99 (m, 2H), 3.92-3.85 (m, 4H), 3.19-3.14 (m, 1H), 2.96-2.90 (m, 1H), 2.83-2.76 (m, 1H), 2.70-2.65 (m, 1H), 1.99-1.97 (m, 3H).

### EXAMPLE 266

### 1-[6,7-Dichloro-10-(1H-pyrazol-4-yl)-3,4-dihydro-1H-pyrazino[1,2-a]indol-2-yl]-2-(1H-pyrazol-5-yl)ethanone

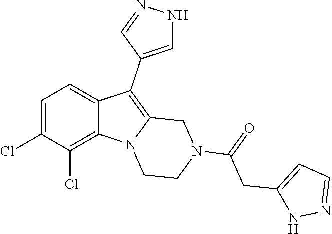 [SMILES: O=C(Cc1ccn[nH]1)N1CCn2c(c(-c3cn[nH]c3)c3ccc(Cl)c(Cl)c32)C1]

Dilute 6,7-dichloro-10-(1H-pyrazol-4-yl)-1,2,3,4-tetrahydropyrazino[1,2-a]indole hydrochloride (100 mg, 0.293 mmol) with DMF (5.5 mL). Add 2-(1H-pyrazol-5-yl)acetic acid (61.1 mg, 0.460 mmol), HATU (213 mg, 0.544 mmol) and DIPEA (0.3 mL, 2 mmol). Stir at ambient temperature for 72 hrs. Quench the reaction with water, and extract with EtOAc. Wash the organic layer with water (3×). Dry the organic layer over magnesium sulfate, filter, and concentrate under vacuum. Purify by reverse phase column chromatography to give 1-[6,7-dichloro-10-(1H-pyrazol-4-yl)-3,4-dihydro-1H-pyrazino[1,2-a]indol-2-yl]-2-(1H-pyrazol-5-yl)ethanone (40.9 mg, 34%). ES/MS (m/z): (³⁵Cl/³⁷Cl) 415.0/417.0 (M+H). ¹H NMR (DMSO-d6): 13.20-13.17 (m, 1H), 12.79-12.74 (m, 1H), 8.10-8.08 (m, 1H), 7.87-7.85 (m, 1H), 7.66-7.60 (m, 2H), 7.28 (d, J=8.5 Hz, 1H), 6.12-6.05 (m, 1H), 5.07-4.91 (m, 2H), 4.69-4.64 (m, 2H), 4.10-3.98 (m, 2H), 3.89-3.87 (m, 2H).

### EXAMPLE 267

### 1-[6,7-Dichloro-10-(1H-pyrazol-4-yl)-3,4-dihydro-1H-pyrazino[1,2-a]indol-2-yl]-2-pyrazin-2-yl-ethanone

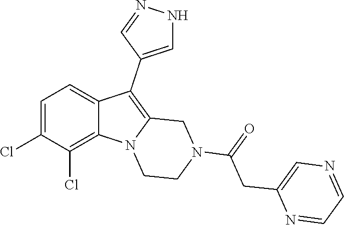 [SMILES: O=C(Cc1cnccn1)N1CCn2c(c(-c3cn[nH]c3)c3ccc(Cl)c(Cl)c32)C1]

Dilute 6,7-dichloro-10-(1H-pyrazol-4-yl)-1,2,3,4-tetrahydropyrazino[1,2-a]indole hydrochloride (95.1 mg, 0.277 mmol) with DMF (5 mL). Add 2-(pyrazin-2-yl)acetic acid (66 mg, 0.46 mmol), HATU (250 mg, 0.638 mmol) and DIPEA (0.3 mL, 2 mmol). Stir at ambient temperature for 72 hrs. Quench the reaction with water, and extract with EtOAc. Wash the organic layer with water (3×). Dry the organic layer over magnesium sulfate, filter, and concentrate under vacuum. Purify by reverse phase column chromatography to give 1-[6,7-dichloro-10-(1H-pyrazol-4-yl)-3,4-dihydro-1H-pyrazino[1,2-a]indol-2-yl]-2-pyrazin-2-yl-ethanone (35.7 mg, 30%). ES/MS (m/z): (³⁵Cl/³⁷Cl) 427.0/429.0 (M+H). ¹H NMR (DMSO-d6): 13.15-13.14 (m, 1H), 8.61-8.58 (m, 3H), 8.08-8.06 (m, 1H), 7.83-7.81 (m, 1H), 7.68-7.67 (m, 1H), 7.29 (d, J=8.5 Hz, 1H), 5.12-4.94 (m, 2H), 4.81-4.78 (m, 2H), 4.15-4.14 (m, 3H), 4.03-4.01 (m, 1H).

### EXAMPLE 268

### 2-[[2-Acetyl-6,7-dichloro-10-(1H-pyrazol-4-yl)-3,4-dihydro-1H-pyrazino[1,2-a]indol-9-yl]oxy]acetamide

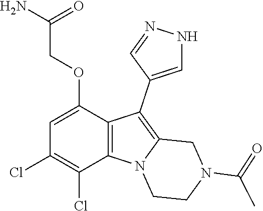 [SMILES: CC(=O)N1CCn2c(c(-c3cn[nH]c3)c3c(OCC(N)=O)cc(Cl)c(Cl)c32)C1]

Dilute 2-[[2-acetyl-6,7-dichloro-10-(1H-pyrazol-4-yl)-3,4-dihydro-1H-pyrazino[1,2-a]indol-9-yl]oxy]acetonitrile (20.7 mg, 0.0512 mmol) and potassium carbonate (30 mg, 0.22 mmol) with DMSO (0.5 mL). Cool to 0° C. Add hydrogen peroxide (0.05 mL, 0.5 mmol, 35% in water) and stir for 2 hrs. at ambient temperature. Concentrate under vacuum and purify by reverse phase column chromatography to give 2-[[2-acetyl-6,7-dichloro-10-(1H-pyrazol-4-yl)-3,4-dihydro-1H-pyrazino[1,2-a]indol-9-yl]oxy]acetamide (4.4 mg, 20%). ES/MS (m/z): (³⁵Cl/³⁷Cl) 422.2/424.2 (M+H). ¹H NMR (DMSO-d6): 13.00-12.96 (m, 1H), 8.00-7.98 (m, 2H), 7.44-7.39 (m, 1H), 6.77-6.72 (m, 2H), 4.79-4.73 (m, 3H), 4.67-4.64 (m, 1H), 4.48 (s, 2H), 3.96-3.89 (m, 2H), 2.12-2.06 (m, 3H).

### EXAMPLE 269

### 2-[[6,7-Dichloro-2-(2-hydroxy acetyl)-10-(1H-pyrazol-4-yl)-3,4-dihydro-1H-pyrazino[1,2-a]indol-9-yl]oxy]acetamide

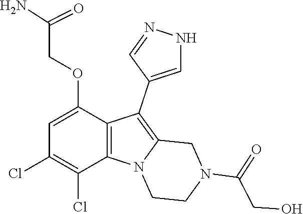 [SMILES: NC(=O)COc1cc(Cl)c(Cl)c2c1c(-c1cn[nH]c1)c1n2CCN(C(=O)CO)C1]

Dilute 2-[[6,7-dichloro-2-(2-hydroxyacetyl)-10-(1H-pyrazol-4-yl)-3,4-dihydro-1H-pyrazino[1,2-a]indol-9-yl]oxy]acetonitrile (21.1 mg, 0.050 mmol) and potassium carbonate (88 mg, 0.64 mmol) with DMSO (3 mL). Cool to 0° C. Add hydrogen peroxide (0.1 mL, 1 mmol, 35% in water and stir for 2 hrs., at ambient temperature. Concentrate under vacuum and purify by reverse phase column chromatography to give 2-[[6,7-dichloro-2-(2-hydroxyacetyl)-10-(1H-pyrazol-4-yl)-3,4-dihydro-1H-pyrazino[1,2-a]indol-9-yl]oxy]acetamide (2.5 mg, 11%). ES/MS (m/z): (³⁵Cl/³⁷Cl) 438.0/440.0 (M+H). 1-E1 NMR (DMSO-d6): 13.08-13.06 (m, 1H), 7.89-7.87 (m, 2H), 7.44-7.41 (m, 1H), 6.76-6.71 (m, 2H), 4.87-4.69 (m, 5H), 4.48 (s, 2H), 4.21-4.16 (m, 2H), 3.90 (s, 2H).

### EXAMPLES 270 & 271

### 4-[[6,7-Dichloro-3-(1H-pyrazol-4-yl)-1H-indol-4-yl]oxy]cyclopentane-1,3-diol

### Isomer 1 and Isomer 2

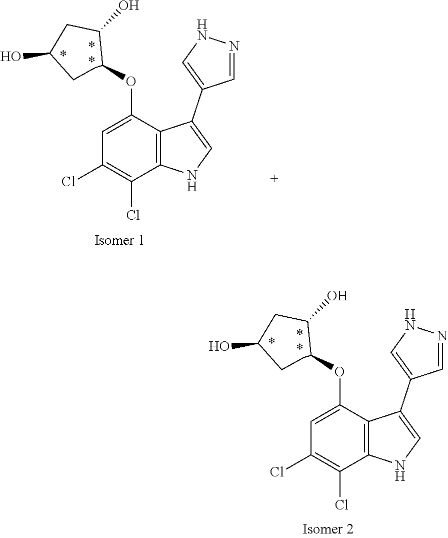

Purify 4-[[6,7-dichloro-3-(1H-pyrazol-4-yl)-1H-indol-4-yl]oxy]cyclopentane-1,3-diol (42 mg, 0.11 mmol) by supercritical fluid chromatography to give the titled compounds (Isomer 1-17.2 mg, 41%; Isomer 2-15.8 mg, 38%). Retention time (isomer 1)=2.89 min (99% ee) and retention time (isomer 2)=4.06 min (98% ee). Column: Chiralpak AD-H, 21×250 mm; Mobile Phase: 25% EtOH: 75% CO₂; Flow Rate: 100 mL/min; Column temperature: 40° C.; Detection: 225 nM. ES/MS (m/z): (³⁵Cl/³⁷Cl) 368.2/370.2 (M+H). ¹H NMR (DMSO-d6): 12.65 (s, 1H), 11.60 (d, J=2.1 Hz, 1H), 8.11-8.08 (m, 2H), 7.45 (d, J=2.6 Hz, 1H), 6.74 (s, 1H), 5.12 (d, J=4.8 Hz, 1H), 4.73 (d, J=3.9 Hz, 1H), 4.53-4.49 (m, 1H), 4.35-4.30 (m, 1H), 4.28-4.23 (m, 1H), 2.57 (dd, J=7.1, 14.0 Hz, 1H), 1.89-1.75 (m, 2H), 1.55 (dt, J=14.0, 4.7 Hz, 1H).

### Intermediate 643

### 1-[9-Amino-6,7-dichloro-10-(1H-pyrazol-4-yl)-3,4-dihydro-1H-pyrazino[1,2-a]indol-2-yl]-2-[tert-butyl(dimethyl)silyl]oxy-ethanone

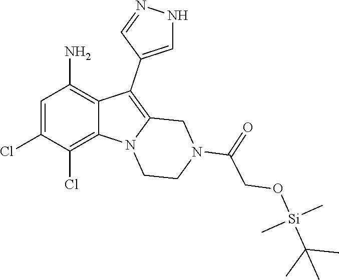 [SMILES: CC(C)(C)[Si](C)(C)OCC(=O)N1CCn2c(c(-c3cn[nH]c3)c3c(N)cc(Cl)c(Cl)c32)C1]

Dilute tert-butyl 9-(tert-butoxycarbonylamino)-6,7-dichloro-10-(1-tetrahydropyran-2-ylpyrazol-4-yl)-3,4-dihydro-1H-pyrazino[1,2-a]indole-2-carboxylate (1.02 g, 1.68 mmol) in HCl (16 mL, 64 mmol, 4M in dioxane) and 1,4-dioxane (16 mL). Stir at ambient temperature for 2 hrs. Concentrate the reaction mixture and dilute with DMF (16 mL). Add HATU (0.917 g, 2.36 mmol), 2-[tert-butyl(dimethyl)silyl]oxyacetic acid (0.42 g, 2.2 mmol), DIPEA (0.9 mL, 5 mmol) and stir at ambient temperature for 45 min. Dilute with EtOAc and wash with water (3×). Dry over magnesium sulfate, filter, and concentrate under vacuum. Purify by flash silica column chromatography eluting with EtOAc in hexanes Further purify by reverse phase column chromatography to give 1-[9-amino-6,7-dichloro-10-(1H-pyrazol-4-yl)-3,4-dihydro-1H-pyrazino[1,2-a]indol-2-yl]-2-[tert-butyl(dimethyl)silyl]oxy-ethanone (35 mg, 4%). ES/MS (m/z): (³⁵Cl/³⁷Cl) 493.8/495.8 (M+H).

### EXAMPLE 272

### N-[6,7-Dichloro-2-(2-hydroxyacetyl)-10-(1H-pyrazol-4-yl)-3,4-dihydro-1H-pyrazino[1,2-a]indol-9-yl]-2,2-difluoro-acetamide

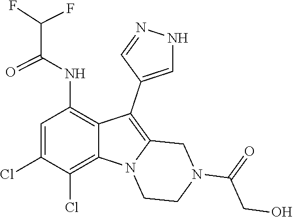 [SMILES: O=C(Nc1cc(Cl)c(Cl)c2c1c(-c1cn[nH]c1)c1n2CCN(C(=O)CO)C1)C(F)F]

Dissolve 1-[9-amino-6,7-dichloro-10-(1H-pyrazol-4-yl)-3,4-dihydro-1H-pyrazino[1,2-a]indol-2-yl]-2-[tert-butyl(dimethyl)silyl]oxy-ethanone (35 mg, 0.071 mmol) in DCM (1.0 mL). Add 4-dimethylamino pyridine (5.6 mg, 0.044 mmol) and cool to 0° C. Add pyridine (0.05 mL, 0.6 mmol) and difluoroacetic anhydride (0.02 mL, 0.2 mmol). Stir for 10 min then remove the ice bath and stir for 16-18 hrs. Concentrate, dilute in DCM (1.0 mL) and add TFA (0.5 mL). Stir for 1.5 hrs., at ambient temperature. Concentrate under vacuum and purify by reverse phase column chromatography to give N-[6, 7-dichloro-2-(2-hydroxy acetyl)-10-(1H-pyrazol-4-yl)-3,4-dihydro-1H-pyrazino[1,2-a]indol-9-yl]-2,2-difluoro-acetamide (11.9 mg, 37%). ES/MS (m/z): (³⁵Cl/³⁷Cl) 458.2/460.2 (M+H). ¹H NMR (DMSO-d6): 13.13-13.12 (m, 1H), 7.82-7.81 (m, 2H), 7.51 (s, 1H), 6.09-5.82 (m, 1H), 4.90-4.87 (m, 1H), 4.77 (s, 1H), 4.73-4.69 (m, 3H), 4.22-4.12 (m, 2H), 3.92 (s, 2H).

### Intermediate 643

### [9-Amino-6,7-dichloro-10-(1H-pyrazol-4-yl)-3,4-dihydro-1H-pyrazino[1,2-a]indol-2-yl]-[(2S)-1,4-dioxan-2-yl]methanone

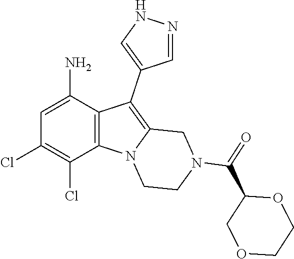 [SMILES: Nc1cc(Cl)c(Cl)c2c1c(-c1cn[nH]c1)c1n2CCN(C(=O)[C@@H]2COCCO2)C1]

Dilute 6,7-dichloro-10-(1H-pyrazol-4-yl)-1,2,3,4-tetrahydropyrazino[1,2-a]indol-9-amine hydrochloride (180 mg, 0.504 mmol) with DMF (6 mL). Add (25)-1,4-dioxane-2-carboxylic acid (77 mg, 0.58 mmol), HATU (295 mg, 0.76 mmol) and DIPEA (0.55 mL, 3.15 mmol). Stir at ambient temperature for 3 hrs. Dilute with saturated aqueous NaHCO₃ and extract with EtOAc. Dry over magnesium sulfate, filter, and concentrate under vacuum. Purify by HPLC to give [9-amino-6,7-dichloro-10-(1H-pyrazol-4-yl)-3,4-dihydro-1H-pyrazino[1,2-a]indol-2-yl]-[(2S)-1,4-dioxan-2-yl]methanone (103 mg, 47%). ES/MS (m/z): (³⁵Cl/³⁷Cl) 434.2/436.2 (M+H).

### EXAMPLE 273

### N-[6,7-Dichloro-2-[(2 S)-1,4-dioxane-2-carbonyl]-10-(1H-pyrazol-4-yl)-3,4-dihydro-1H-pyrazino[1,2-a]indol-9-yl]-2,2-difluoro-acetamide

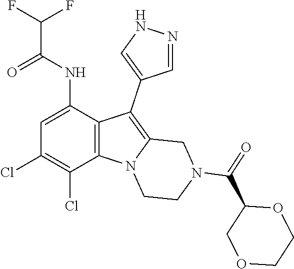 [SMILES: O=C(Nc1cc(Cl)c(Cl)c2c1c(-c1cn[nH]c1)c1n2CCN(C(=O)[C@@H]2COCCO2)C1)C(F)F]

Dissolve [9-amino-6,7-dichloro-10-(1H-pyrazol-4-yl)-3,4-dihydro-1H-pyrazino[1,2-a]indol-2-yl]-[(2S)-1,4-dioxan-2-yl]methanone (50 mg, 0.115 mmol) in 1,4-dioxane (2 mL). Add DMAP (25 mg, 0.20 mmol), pyridine (0.10 mL, 1.0 mmol) and difluoroacetic anhydride (0.060 mL, 0.46 mmol). Stir at ambient temperature for 16 hrs. Dilute with saturated aqueous NaHCO₃ and extract with EtOAc. Dry over magnesium sulfate, filter, concentrate under vacuum and purify by HPLC to give N-[6,7-dichloro-2-[(2S)-1,4-dioxane-2-carbonyl]-10-(1H-pyrazol-4-yl)-3,4-dihydro-1H-pyrazino[1,2-a]indol-9-yl]-2,2-difluoro-acetamide (25 mg, 42%). ES/MS (m/z): (³⁵Cl/³⁷Cl) 514.0/516.0 (M+H).

### Intermediate 644

### 3-[2-[9-Amino-6,7-dichloro-10-(1H-pyrazol-4-yl)-3,4-dihydro-1H-pyrazino[1,2-a]indol-2-yl]-2-oxo-ethyl]oxazolidin-2-one

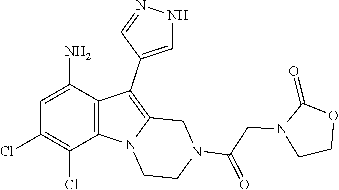 [SMILES: Nc1cc(Cl)c(Cl)c2c1c(-c1cn[nH]c1)c1n2CCN(C(=O)CN2CCOC2=O)C1]

Dilute 6,7-dichloro-10-(1H-pyrazol-4-yl)-1,2,3,4-tetrahydropyrazino[1,2-a]indol-9-amine hydrochloride (180 mg, 0.504 mmol) with DMF (6 mL). Add 2-(2-oxooxazolidin-3-yl)acetic acid (90 mg, 0.62 mmol). Add HATU (307 mg, 0.79 mmol) and DIPEA (0.55 mL, 3.15 mmol). Stir at ambient temperature for 3 hrs. Quench with saturated aqueous NaHCO₃, extract with EtOAc, dry over magnesium sulfate, filter, and concentrate under vacuum. Purify by HPLC to give 3-[2-[9-amino-6,7-dichloro-10-(1H-pyrazol-4-yl)-3,4-dihydro-1H-pyrazino[1,2-a]indol-2-yl]-2-oxo-ethyl]oxazolidin-2-one (82 mg, 36%). ES/MS (m/z): (³⁵Cl/³⁷Cl) 449.2/451.2 (M+H).

### EXAMPLE 274

### N-[6,7-Dichloro-2-[2-(2-oxooxazolidin-3-yl)acetyl]-10-(1H-pyrazol-4-yl)-3,4-dihydro-1H-pyrazino[1,2-a]indol-9-yl]-2,2-difluoro-acetamide

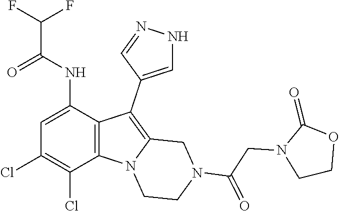 [SMILES: O=C(Nc1cc(Cl)c(Cl)c2c1c(-c1cn[nH]c1)c1n2CCN(C(=O)CN2CCOC2=O)C1)C(F)F]

Dissolve 3-[2-[9-amino-6,7-dichloro-10-(1H-pyrazol-4-yl)-3,4-dihydro-1H-pyrazino[1,2-a]indol-2-yl]-2-oxo-ethyl]oxazolidin-2-one (40 mg, 0.089 mmol) in 1,4-dioxane (2 mL). Add DMAP (15 mg, 0.12 mmol), pyridine (0.10 mL, 1.0 mmol) and difluoroacetic anhydride (0.030 mL, 0.23 mmol). Stir at ambient temperature for 48 hrs. Quench with saturated aqueous NaHCO₃ and extract with EtOAc. Dry over magnesium sulfate, filter, concentrate under vacuum, and purify by HPLC to give N-[6,7-dichloro-2-[2-(2-oxooxazolidin-3-yl)acetyl]-10-(1H-pyrazol-4-yl)-3,4-dihydro-1H-pyrazino[1,2-a]indol-9-yl]-2,2-difluoro-acetamide (24 mg, 53%). ES/MS (m/z): (³⁵Cl/³⁷Cl) 527.0/529.0 (M+H). ¹H NMR (DMSO-d6): 13.10-13.09 (m, 1H), 9.77 (s, 1H), 7.88-7.85 (m, 1H), 7.51 (s, 2H), 6.10-5.83 (m, 1H), 4.82-4.72 (m, 4H), 4.31-4.25 (m, 4H), 3.97 (t, J=4.8 Hz, 2H), 3.55 (t, J=8.0 Hz, 2H).

### Intermediate 645

### 4-[9-Amino-6,7-dichloro-10-(1H-pyrazol-4-yl)-3,4-dihydro-1H-pyrazino[1,2-a]indol-2-yl]-N,N-dimethyl-4-oxo-butanamide

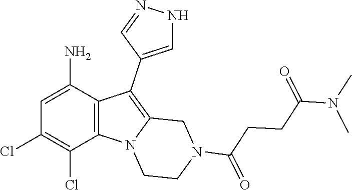 [SMILES: CN(C)C(=O)CCC(=O)N1CCn2c(c(-c3cn[nH]c3)c3c(N)cc(Cl)c(Cl)c32)C1]

Dilute together 6,7-dichloro-10-(1H-pyrazol-4-yl)-1,2,3,4-tetrahydropyrazino[1,2-a]indol-9-amine hydrochloride (180 mg, 0.504 mmol) with DMF (6 mL). Add 4-(dimethylamino)-4-oxo-butanoic acid (91 mg, 0.63 mmol), HATU (309 mg, 0.80 mmol) and DIPEA (0.55 mL, 3.15 mmol). Stir at ambient temperature for 2.5 hrs. Quench with saturated aqueous NaHCO₃ and extract with EtOAc. Dry over magnesium sulfate, filter, concentrate under vacuum and purify via reverse phase HPLC chromatography to give 4-[9-amino-6,7-dichloro-10-(1H-pyrazol-4-yl)-3,4-dihydro-1H-pyrazino[1,2-a]indol-2-yl]-N,N-dimethyl-4-oxo-butanamide (60 mg, 27%). ES/MS (m/z): (³⁵Cl/³⁷Cl) 449.2/451.2 (M+H).

### EXAMPLE 275

### 4-[6,7-Dichloro-9-[(2,2-difluoroacetyl)amino]-10-(1H-pyrazol-4-yl)-3,4-dihydro-1H-pyrazino[1,2-a]indol-2-yl]-N,N-dimethyl-4-oxo-butanamide

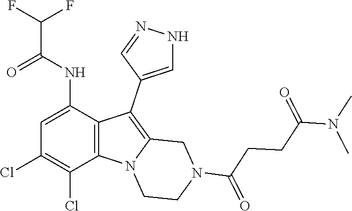 [SMILES: CN(C)C(=O)CCC(=O)N1CCn2c(c(-c3cn[nH]c3)c3c(NC(=O)C(F)F)cc(Cl)c(Cl)c32)C1]

Dissolve 4-[9-amino-6,7-dichloro-10-(1H-pyrazol-4-yl)-3,4-dihydro-1H-pyrazino[1,2-a]indol-2-yl]-N,N-dimethyl-4-oxo-butanamide (57 mg, 0.13 mmol) in 1,4-dioxane (2 mL). Add DMAP (15 mg, 0.12 mmol), pyridine (0.10 mL, 1.0 mmol) and difluoroacetic anhydride (0.030 mL, 0.23 mmol). Stir at ambient temperature for 20 hrs. Quench with saturated aqueous NaHCO₃ and extract with EtOAc. Dry over magnesium sulfate, filter, and concentrate under vacuum. Purify by HPLC chromatography to give 4-[6,7-dichloro-9-[(2,2-difluoroacetyl)amino]-10-(1H-pyrazol-4-yl)-3,4-dihydro-1H-pyrazino[1,2-a]indol-2-yl]-N,N-dimethyl-4-oxo-butanamide (33 mg, 49%). ES/MS (m/z): (³⁵Cl/³⁷Cl) 527.0/529.0 (M+H). ¹H NMR (DMSO): 13.07-13.04 (m, 1H), 9.80-9.73 (m, 1H), 7.93-7.91 (m, 1H), 7.52 (s, 2H), 6.08-5.81 (m, 1H), 4.79 (s, 2H), 4.67 (s, 2H), 4.05-3.93 (m, 2H), 2.97 (s, 3H), 2.78 (s, 3H), 2.67-2.61 (m, 2H), 2.54-2.49 (m, 2H).

### Intermediate 646

### 4-[2-[9-Amino-6,7-dichloro-10-(1H-pyrazol-4-yl)-3,4-dihydro-1H-pyrazino[1,2-a]indol-2-yl]-2-oxo-ethyl]piperidin-2-one

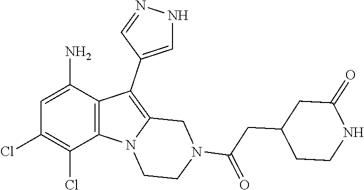 [SMILES: Nc1cc(Cl)c(Cl)c2c1c(-c1cn[nH]c1)c1n2CCN(C(=O)CC2CCNC(=O)C2)C1]

Dilute 6,7-dichloro-10-(1H-pyrazol-4-yl)-1,2,3,4-tetrahydropyrazino[1,2-a]indol-9-amine hydrochloride (156 mg, 0.435 mmol) with DMF (6 mL). Add 2-(2-oxopiperidin-4-yl)acetic acid (84 mg, 0.51 mmol), HATU (255 mg, 0.66 mmol) and DIPEA (0.25 mL, 1.43 mmol). Stir at ambient temperature for 5 hrs., then add additional DIPEA (0.25 mL, 1.43 mmol). Stir at ambient temperature for 18 hrs. Load the reaction mixture onto a 10 g cationic ion exchange resin. Elute first with a 1:1 mixture of MeOH and DCM. Next, collect separately the fraction from eluting with 7N ammonia in MeOH. Concentrate this fraction to give crude 4-[2-[9-amino-6,7-dichloro-10-(1H-pyrazol-4-yl)-3,4-dihydro-1H-pyrazino[1,2-a]indol-2-yl]-2-oxo-ethyl]piperidin-2-one. (241 mg, 99+%). ES/MS (m/z): (³⁵Cl/³⁷Cl) 459.2/461.2 (M+H).

### EXAMPLE 276

### N-[6,7-Dichloro-2-[2-(2-oxo-4-piperidyl)acetyl]-10-(1H-pyrazol-4-yl)-3,4-dihydro-1H-pyrazino[1,2-a]indol-9-yl]-2,2-difluoro-acetamide

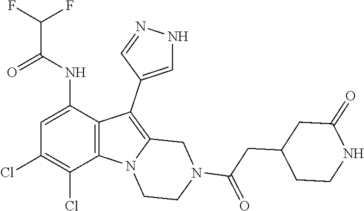 [SMILES: O=C1CC(CC(=O)N2CCn3c(c(-c4cn[nH]c4)c4c(NC(=O)C(F)F)cc(Cl)c(Cl)c43)C2)CCN1]

Dilute 4-[2-[9-amino-6,7-dichloro-10-(1H-pyrazol-4-yl)-3,4-dihydro-1H-pyrazino[1,2-a]indol-2-yl]-2-oxo-ethyl]piperidin-2-one (201 mg, 0.435 mmol) with DCM (5 mL) and 1,4-dioxane (2 mL). Add DMAP (30 mg, 0.24 mmol) and cool to 0° C. Add pyridine (0.10 mL, 1.0 mmol) and difluoroacetic anhydride (0.060 mL, 0.46 mmol). Stir for 10 min at 0° C. then stir for 16 hrs., at ambient temperature. Add 1,4-dioxane (5 mL), 1,2-dimethoxyethane (5 mL), DIPEA (0.15 mL, 0.86 mmol) and difluoroacetic anhydride (0.06 mL, 0.46 mmol). Stir for 3 hrs. Add difluoroacetic anhydride (0.10 mL, 0.77 mmol) and stir for 2 hrs. Quench with MeOH and aqueous 1M NaOH. Dilute with saturated aqueous NaHCO₃ and extract with EtOAc. Dry over magnesium sulfate, filter, concentrate under vacuum, and purify by HPLC to give N-[6,7-dichloro-2-[2-(2-oxo-4-piperidyl)acetyl]-10-(1H-pyrazol-4-yl)-3,4-dihydro-1H-pyrazino[1,2-a]indol-9-yl]-2,2-difluoro-acetamide (38 mg, 16%). ES/MS (m/z): (³⁵Cl/³⁷Cl) 539.0/541.0 (M+H).

### Intermediate 647

### 1-[9-Amino-6,7-dichloro-10-(1H-pyrazol-4-yl)-3,4-dihydro-1H-pyrazino[1,2-a]indol-2-yl]-2-pyrazin-2-yl-ethanone

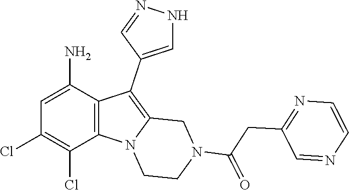 [SMILES: Nc1cc(Cl)c(Cl)c2c1c(-c1cn[nH]c1)c1n2CCN(C(=O)Cc2cnccn2)C1]

Dilute 6,7-dichloro-10-(1H-pyrazol-4-yl)-1,2,3,4-tetrahydropyrazino[1,2-a]indol-9-amine hydrochloride (156 mg, 0.435 mmol) and DMF (6 mL). Add 2-(pyrazin-2-yl)acetic acid (74 mg, 0.52 mmol). Add HATU (273 mg, 0.70 mmol) then add DIEA (0.25 mL, 1.43 mmol). Stir at ambient temperature for 7 hrs. Add additional DIEA (0.25 mL, 1.43 mmol) and stir for 16 hrs. Purify via silica gel normal phase chromatography eluting with 10% MeOH in DCM to give semi-pure 1-[9-amino-6,7-dichloro-10-(1H-pyrazol-4-yl)-3,4-dihydro-1H-pyrazino[1,2-a]indol-2-yl]-2-pyrazin-2-yl-ethanone. (136 mg, 71%). ES/MS (m/z): (³⁵Cl/³⁷Cl) 442.2/444.2 (M+H).

### EXAMPLE 277

### N-[6,7-Dichloro-2-(2-pyrazin-2-ylacetyl)-10-(1H-pyrazol-4-yl)-3,4-dihydro-1H-pyrazino[1,2-a]indol-9-yl]-2,2-difluoro-acetamide

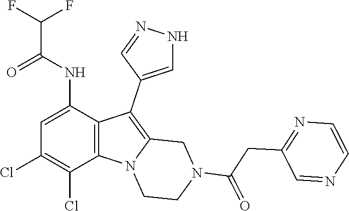 [SMILES: O=C(Nc1cc(Cl)c(Cl)c2c1c(-c1cn[nH]c1)c1n2CCN(C(=O)Cc2cnccn2)C1)C(F)F]

Dilute 1-[9-amino-6,7-dichloro-10-(1H-pyrazol-4-yl)-3,4-dihydro-1H-pyrazino[1,2-a]indol-2-yl]-2-pyrazin-2-yl-ethanone (136 mg, 0.31 mmol) with DCM (4 mL) and 1,4-dioxane (1 mL). Add DMAP (29 mg, 0.23 mmol) and cool to 0° C. Add pyridine (0.10 mL, 1.0 mmol) and difluoroacetic anhydride (0.050 mL, 0.38 mmol) and stir for 10 minutes. Stir at ambient temperature for 16 hrs. Add difluoroacetic anhydride (0.02 mL, 0.15 mmol). Stir for 3 hrs. Purify by flash column chromatography eluting with MeOH in DCM to give N-[6,7-dichloro-2-(2-pyrazin-2-ylacetyl)-10-(1H-pyrazol-4-yl)-3,4-dihydro-1H-pyrazino[1,2-a]indol-9-yl]-2,2-difluoro-acetamide (5 mg, 3%). ES/MS (m/z): (³⁵Cl/³⁷Cl) 520.2/522.2 (M+H). ¹H NMR (DMSO): 13.26-13.24 (m, 1H), 9.80-9.79 (m, 1H), 8.59-8.50 (m, 3H), 7.74-7.58 (m, 2H), 7.53 (s, 1H), 6.07-5.80 (m, 1H), 4.89-4.80 (m, 2H), 4.72 (s, 2H), 4.13 (s, 2H), 4.11-3.96 (m, 2H).

### EXAMPLE 278

### 5,6-Dichloro-9-(1H-pyrazol-4-yl)-2,3-dihydro-1H-pyrrolo[1,2-a]indol-2-ol

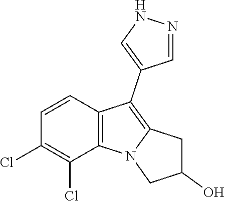 [SMILES: OC1Cc2c(-c3cn[nH]c3)c3ccc(Cl)c(Cl)c3n2C1]

Dissolve 7,8-dichloro-4-(1H-pyrazol-4-yl)-1,3-dihydropyrrolo[1,2-a]indol-2-one (0.034, 0.11 mmol) in THF (3 mL). Add sodium borohydride (70 mg, 1.85 mmol). Stir at ambient temperature for 24 hrs. Add additional sodium borohydride (30 mg, 0.79 mmol) and stir for 6 hrs. Quench with saturated aqueous NaHCO₃ and extract with EtOAc. Dry over magnesium sulfate, filter, and concentrate under vacuum. Purify by flash column chromatography eluting with EtOAc in hexanes to give 5,6-dichloro-9-(1H-pyrazol-4-yl)-2,3-dihydro-1H-pyrrolo[1,2-a]indol-2-ol (28 mg, 82%). ES/MS (m/z): (³⁵Cl/³⁷Cl) 308.0/310.0 (M+H). ¹H NMR (DMSO-d6): 13.01-13.00 (m, 1H), 8.13-8.11 (m, 2H), 7.71 (d, J=8.5 Hz, 1H), 7.19 (d, J=8.5 Hz, 1H), 5.57 (d, J=4.4 Hz, 1H), 4.97-4.95 (m, 1H), 4.58 (dd, J=5.2, 11.2 Hz, 1H), 4.34 (dd, J=2.2, 11.2 Hz, 1H), 3.40-3.33 (m, 1H), 2.94 (dd, J=2.4, 16.9 Hz, 1H).

### EXAMPLE 279

### 2-((6,7-Dichloro-2-(2-methoxyacetyl)-10-(1H-pyrazol-4-yl)-1,2,3,4-tetrahydropyrazino[1,2-a]indol-9-yl)oxy)acetonitrile

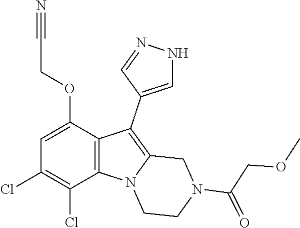 [SMILES: COCC(=O)N1CCn2c(c(-c3cn[nH]c3)c3c(OCC#N)cc(Cl)c(Cl)c32)C1]

Dissolve 2-((6,7-dichloro-10-(1H-pyrazol-4-yl)-1,2,3,4-tetrahydropyrazino[1,2-a]indol-9-yl)oxy)acetonitrile (75 mg, 0.23 mmol, HCl salt) in DMF (3 mL). Add TEA (0.10 mL, 0.72 mmol) and acetyl chloride (0.030 mL, 0.41 mmol) at 0° C. Stir 2 hrs., at ambient temperature. Quench with saturated aqueous NaHCO₃ and extract with EtOAc, wash with saturated aqueous NaCl, dry over anhydrous Na₂SO₄, filter, and concentrate under vacuum. Purify by prep-HPLC to give 2-((6,7-dichloro-2-(2-methoxyacetyl)-10-(1H-pyrazol-4-yl)-1,2, 3,4-tetrahydropyrazino[1,2-a]indol-9-yl)oxy)acetonitrile (30 mg, 37%) as an off-white solid. ES/MS (m/z): (³⁵Cl/³⁷Cl) 434.0/436.0 (M+H).

Examples 280-296 (Table 4) were prepared by similar means from 2-((6,7-dichloro-10-(1H-pyrazol-4-yl)-1,2,3,4-tetrahydropyrazino[1,2-a]indol-9-yl)oxy)acetonitrile using an appropriate electrophile in DMF or DCM in the presence of a base (e.g. TEA, DIPEA). Carboxylic acids were activated using HATU.

TABLE 4

| Example | Chemical Name | Structure | Analytical Data |
|---|---|---|---|
| 280 | 2-((2-Acetyl-6,7-dichloro-10- (1H-pyrazol-4-yl)-1,2,3,4- tetrahydropyrazino[1,2-a]indol- 9-yl)oxy)acetonitrile | 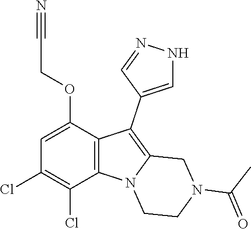 [SMILES: CC(=O)N1CCn2c(c(-c3cn[nH]c3)c3c(OCC#N)cc(Cl)c(Cl)c32)C1] | ES/MS (m/z): (³⁵Cl/³⁷Cl) 404.0/406.2 (M + H) |
| 281 | (R)-2-((6,7-Dichloro-2-(4- methylmorpholine-2-carbonyl)- 10-(1H-pyrazol-4-yl)-1,2,3,4- tetrahydropyrazino[1,2-a]indol- 9-yl)oxy)acetonitrile | 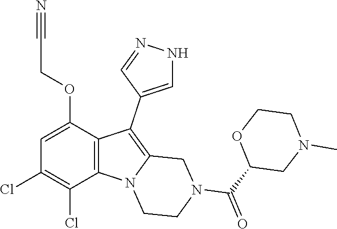 [SMILES: CN1CCO[C@@H](C(=O)N2CCn3c(c(-c4cn[nH]c4)c4c(OCC#N)cc(Cl)c(Cl)c43)C2)C1] | ES/MS (m/z): (³⁵Cl/³⁷Cl) 489.2/491.2 (M + H) |
| 282 | 2-((6,7-Dichloro-2-(5-methyl- 1,2,4-oxadiazole-3-carbonyl)- 10-(1H-pyrazol-4-yl)-1,2,3,4- tetrahydropyrazino[1,2-a]indol- 9-yl)oxy)acetonitrile | 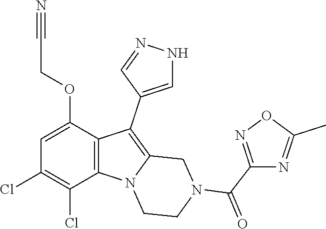 [SMILES: Cc1nc(C(=O)N2CCn3c(c(-c4cn[nH]c4)c4c(OCC#N)cc(Cl)c(Cl)c43)C2)no1] | ES/MS (m/z): (³⁵Cl/³⁷Cl) 472.0/474.2 (M + H) |
| 283 | 2-((6,7-Dichloro-2-(1-methyl- 1H-1,2,4-triazole-3-carbonyl)- 10-(1H-pyrazol-4-yl)-1,2,3,4- tetrahydropyrazino[1,2-a]indol- 9-yl)oxy)acetonitrile | 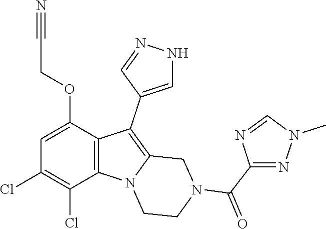 [SMILES: Cn1cnc(C(=O)N2CCn3c(c(-c4cn[nH]c4)c4c(OCC#N)cc(Cl)c(Cl)c43)C2)n1] | ES/MS (m/z): (³⁵Cl/³⁷Cl) 471.2/473.2 (M + H) |
| 284 | 2-((6, 7-Dichloro-2-(2-(2- oxooxazolidin-3-yl)acetyl)-10- (1H-pyrazol-4-yl)-1,2,3,4- tetrahydropyrazino[1,2-a]indol- 9-yl)oxy)acetonitrile | 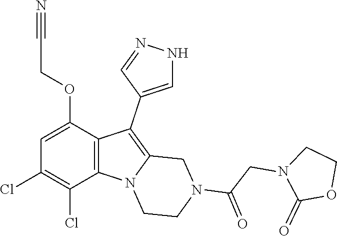 [SMILES: N#CCOc1cc(Cl)c(Cl)c2c1c(-c1cn[nH]c1)c1n2CCN(C(=O)CN2CCOC2=O)C1] | ES/MS (m/z): (³⁵Cl/³⁷Cl) 489.2/491.2 (M + H) |
| 285 | (S)-2-((6,7-Dichloro-2-(1,4- dioxane-2-carbonyl)-10-(1H- pyrazol-4-yl)-1,2,3,4- tetrahydropyrazino[1,2-a]indol- 9-yl)oxy)acetonitrile | 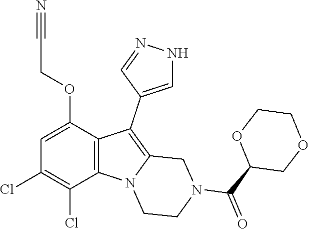 [SMILES: N#CCOc1cc(Cl)c(Cl)c2c1c(-c1cn[nH]c1)c1n2CCN(C(=O)[C@@H]2COCCO2)C1] | ES/MS (m/z): (³⁵Cl/³⁷Cl) 476.2/478.2 (M + H) |
| 286 | 2-((6,7-Dichloro-2-(pyrazine-2- carbonyl)-10-(1H-pyrazol-4-yl)- 1,2,3,4-tetrahydropyrazino[1,2- a]indol-9-yl)oxy)acetonitrile | 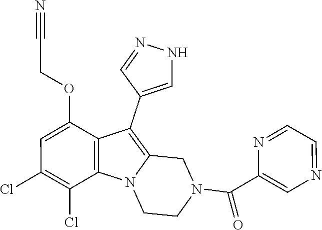 [SMILES: N#CCOc1cc(Cl)c(Cl)c2c1c(-c1cn[nH]c1)c1n2CCN(C(=O)c2cnccn2)C1] | ES/MS (m/z): (³⁵Cl/³⁷Cl) 468.0/470.2 (M + H) |
| 287 | 2-((2-(2-(1H-1,2,4-Triazol-1- yl)acetyl)-6,7-dichloro-10-(1H- pyrazol-4-yl)-1,2,3,4- tetrahydropyrazino[1,2-a]indol- 9-yl)oxy)acetonitrile | 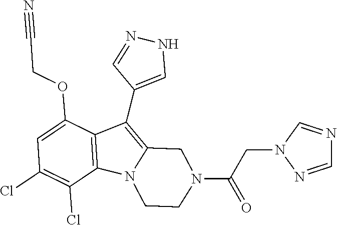 [SMILES: N#CCOc1cc(Cl)c(Cl)c2c1c(-c1cn[nH]c1)c1n2CCN(C(=O)Cn2cncn2)C1] | ES/MS (m/z): (³⁵Cl/³⁷Cl) 471.2/473.2 (M + H) |
| 288 | 2-((6,7-Dichloro-2-(1,4- dioxane-2-carbonyl)-10-(1H- pyrazol-4-yl)-1,2,3,4- tetrahydropyrazino[1,2-a]indol- 9-yl)oxy)acetonitrile | 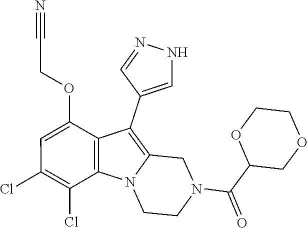 [SMILES: N#CCOc1cc(Cl)c(Cl)c2c1c(-c1cn[nH]c1)c1n2CCN(C(=O)C2COCCO2)C1] | ES/MS (m/z): (³⁵Cl/³⁷Cl) 476.2/478.2 (M + H) |
| 289 | 2-((6,7-Dichloro-2-(2- morpholinoacetyl)-10-(1H- pyrazol-4-yl)-1,2,3,4- tetrahydropyrazino[1,2-a]indol- 9-yl)oxy)acetonitrile | 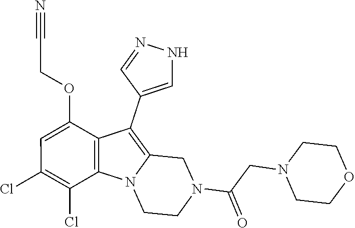 [SMILES: N#CCOc1cc(Cl)c(Cl)c2c1c(-c1cn[nH]c1)c1n2CCN(C(=O)CN2CCOCC2)C1] | ES/MS (m/z): (³⁵Cl/³⁷Cl) 489.2/491.2 (M + H) |
| 290 | 2-((6,7-Dichloro-2-(2-(3- oxomorpholino)acetyl)-10-(1H- pyrazol-4-yl)-1,2,3,4- tetrahydropyrazino[1,2-a]indol- 9-yl)oxy)acetonitrile | 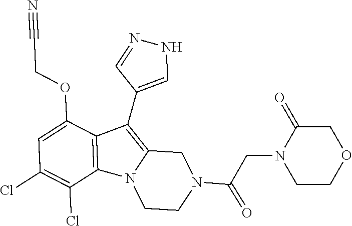 [SMILES: N#CCOc1cc(Cl)c(Cl)c2c1c(-c1cn[nH]c1)c1n2CCN(C(=O)CN2CCOCC2=O)C1] | ES/MS (m/z): (³⁵Cl/³⁷Cl) 503.2/505.2 (M + H) |
| 291 | 2-((6,7-Dichloro-2-(1-methyl-5- oxopyrrolidine-3-carbonyl)-10- (1H-pyrazol-4-yl)-1,2,3,4- tetrahydropyrazino[1,2-a]indol- 9-yl)oxy)acetonitrile | 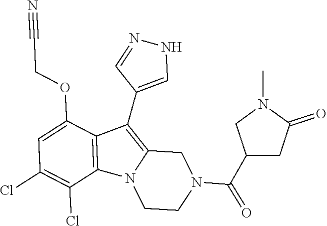 [SMILES: CN1CC(C(=O)N2CCn3c(c(-c4cn[nH]c4)c4c(OCC#N)cc(Cl)c(Cl)c43)C2)CC1=O] | ES/MS (m/z): (³⁵Cl/³⁷Cl) 487.2/489.2 (M + H) |
| 292 | 2-((2-(3-(1H-1,2,4-Triazol-1- yl)propanoyl)-6,7-dichloro-10- (1H-pyrazol-4-yl)-1,2,3,4- tetrahydropyrazino[1,2-a]indol- 9-yl)oxy)acetonitrile | 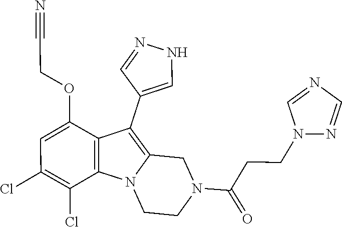 [SMILES: N#CCOc1cc(Cl)c(Cl)c2c1c(-c1cn[nH]c1)c1n2CCN(C(=O)CCn2cncn2)C1] | ES/MS (m/z): (³⁵Cl/³⁷Cl) 485.2/487.2 (M + H) |
| 293 | 2-((6,7-Dichloro-2-(2-(3,5- dimethyl-1H-1,2,4-triazol-1- yl)acetyl)-10-(1H-pyrazol-4-yl)- 1,2,3,4-tetrahydropyrazino[1,2- a]indol-9-yl)oxy)acetonitrile | 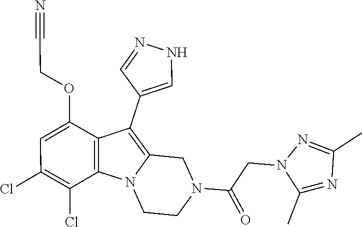 [SMILES: Cc1nc(C)n(CC(=O)N2CCn3c(c(-c4cn[nH]c4)c4c(OCC#N)cc(Cl)c(Cl)c43)C2)n1] | ES/MS (m/z): (³⁵Cl/³⁷Cl) 499.2/501.2 (M + H) |
| 294 | 2-((6,7-Dichloro-2-(4- methylmorpholine-2-carbonyl)- 10-(1H-pyrazol-4-yl)-1,2,3,4- tetrahydropyrazino[1,2-a]indol- 9-yl)oxy)acetonitrile | 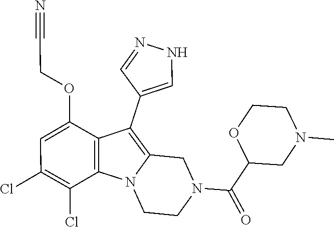 [SMILES: CN1CCOC(C(=O)N2CCn3c(c(-c4cn[nH]c4)c4c(OCC#N)cc(Cl)c(Cl)c43)C2)C1] | ES/MS (m/z): (³⁵Cl/³⁷Cl) 489.2/491.2 (M + H) |
| 295 | 2-((6,7-Dichloro-2-(3- (piperidine-1- carbonyl)pyrazine-2-carbonyl)- 10-(1H-pyrazol-4-yl)-1,2,3,4- tetrahydropyrazino[1,2-a]indol- 9-yl)oxy)acetonitrile | 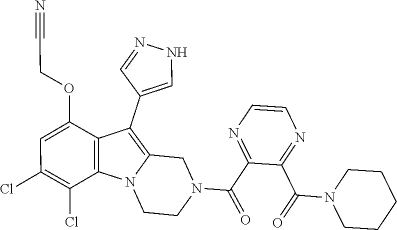 [SMILES: N#CCOc1cc(Cl)c(Cl)c2c1c(-c1cn[nH]c1)c1n2CCN(C(=O)c2nccnc2C(=O)N2CCCCC2)C1] | ES/MS (m/z): (³⁵Cl/³⁷Cl) 579.2/581.2 (M + H) |
| 296 | 2-((6,7-Dichloro-2-(3- (morpholine-4- carbonyl)pyrazine-2-carbonyl)- 10-(1H-pyrazol-4-yl)-1,2,3,4- tetrahydropyrazino[1,2-a]indol- 9-yl)oxy)acetonitrile | 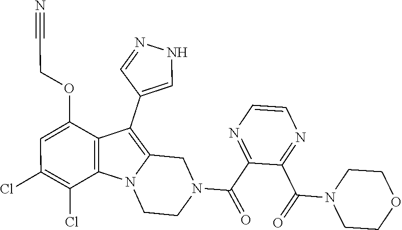 [SMILES: N#CCOc1cc(Cl)c(Cl)c2c1c(-c1cn[nH]c1)c1n2CCN(C(=O)c2nccnc2C(=O)N2CCOCC2)C1] | ES/MS (m/z): (³⁵Cl/³⁷Cl) 581.2/583.2 (M + H) |

### EXAMPLE 297

### 4-(6,7-Dichloro-10-(1H-pyrazol-4-yl)-3,4-dihydropyrazino[1,2-a]indol-2(1H)-yl)-4-oxobutanamide

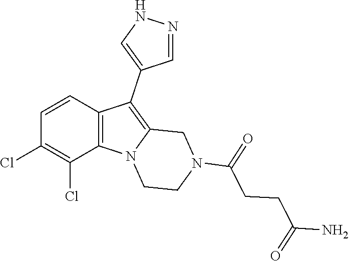 [SMILES: NC(=O)CCC(=O)N1CCn2c(c(-c3cn[nH]c3)c3ccc(Cl)c(Cl)c32)C1]

Dissolve 6,7-dichloro-10-(1H-pyrazol-4-yl)-1,2,3,4-tetrahydropyrazino[1,2-a]indole (100 mg, 0.291 mmol, HCl salt) in DMF (3 mL). Add TEA (0.10 mL, 1.43 mmol), HATU (135 mg, 0.347 mmol), and succinamic acid (42 mg, 0.347 mmol). Stir for 18 hrs. at ambient temperature under N₂. Quench with saturated aqueous NaHCO₃ and extract with EtOAc, wash with saturated aqueous NaCl (3×), dry over anhydrous Na₂SO₄, filter, and concentrate under vacuum. Purify by prep-HPLC to give 4-(6,7-dichloro-10-(1H-pyrazol-4-yl)-3,4-dihydropyrazino[1,2-a]indol-2(1H)-yl)-4-oxobutanamide (55 mg, 45%) as a white solid. ES/MS (m/z): (³⁵Cl/³⁷Cl) 406.0/408.0 (M+H).

Examples 298-335 (Table 5) were prepared by similar means from 6,7-dichloro-10-(1H-pyrazol-4-yl)-1,2,3,4-tetrahydropyrazino[1,2-a]indole using an appropriate electrophile in DMF or DCM in the presence of a base (e.g. TEA, DIPEA). Carboxylic acids were activated using HATU.

TABLE 5

| Example | Chemical Name | Structure | Analytical Data |
|---|---|---|---|
| 298 | 1-(6,7-Dichloro-10-(1H- pyrazol-4-yl)-3,4- dihydropyrazino[1,2-a]indol- 2(1H)-yl)-2-(1,4-dioxepan-6- yl)ethan-1-one | 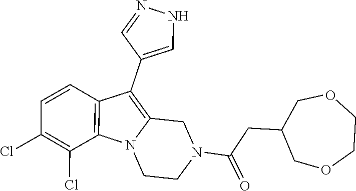 [SMILES: O=C(CC1COCCOC1)N1CCn2c(c(-c3cn[nH]c3)c3ccc(Cl)c(Cl)c32)C1] | ES/MS (m/z): (³⁵Cl/³⁷Cl) 449.2/451.2 (M + H) |
| 299 | 1-(6,7-Dichloro-10-(1H- pyrazol-4-yl)-3,4- dihydropyrazino[1,2-a]indol- 2(1H)-yl)-2-(1,4-dioxan-2- yl)ethan-1-one | 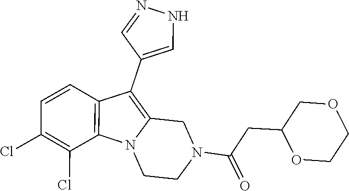 [SMILES: O=C(CC1COCCO1)N1CCn2c(c(-c3cn[nH]c3)c3ccc(Cl)c(Cl)c32)C1] | ES/MS (m/z): (³⁵Cl/³⁷Cl) 435.2/437.2 (M + H) |
| 300 | 3-(2-(6,7-Dichloro-10-(1H- pyrazol-4-yl)-3,4- dihydropyrazino[1,2-a]indol- 2(1H)-yl)-2-oxoethyl)-1- methylpyrrolidin-2-one | 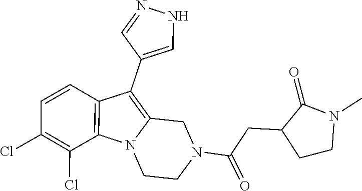 [SMILES: CN1CCC(CC(=O)N2CCn3c(c(-c4cn[nH]c4)c4ccc(Cl)c(Cl)c43)C2)C1=O] | ES/MS (m/z): (³⁵Cl/³⁷Cl) 446.2/448.2 (M + H) |
| 301 | 2-(2-(6,7-Dichloro-10-(1H- pyrazol-4-yl)-3,4- dihydropyrazino[1,2-a]indol- 2(1H)-yl)-2-oxoethoxy)-N,N- dimethylacetamide | 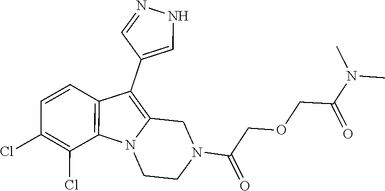 [SMILES: CN(C)C(=O)COCC(=O)N1CCn2c(c(-c3cn[nH]c3)c3ccc(Cl)c(Cl)c32)C1] | ES/MS (m/z): (³⁵Cl/³⁷Cl) 450.2/452.2 (M + H) |
| 302 | 3-(6,7-Dichloro-10-(1H- pyrazol-4-yl)-3,4- dihydropyrazino[1,2-a]indol- 2(1H)-yl)-N,N-dimethyl-3- oxopropanamide | 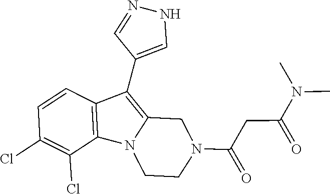 [SMILES: CN(C)C(=O)CC(=O)N1CCn2c(c(-c3cn[nH]c3)c3ccc(Cl)c(Cl)c32)C1] | ES/MS (m/z): (³⁵Cl/³⁷Cl) 420.2/422.2 (M + H) |
| 303 | 4-(6,7-Dichloro-10-(1H- pyrazol-4-yl)-1,2,3,4- tetrahydropyrazino[1,2- a]indole-2-carbonyl)-1- methylpiperidin-2-one | 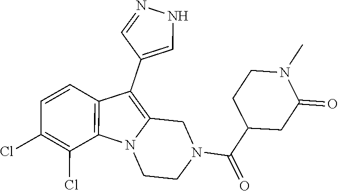 [SMILES: CN1CCC(C(=O)N2CCn3c(c(-c4cn[nH]c4)c4ccc(Cl)c(Cl)c43)C2)CC1=O] | ES/MS (m/z): (³⁵Cl/³⁷Cl) 446.2/448.2 (M + H) |
| 304 | 4-(3-(6,7-Dichloro-10-(1H- pyrazol-4-yl)-3,4- dihydropyrazino[1,2-a]indol- 2(1H)-yl)-3- oxopropyl)morpholin-3-one | 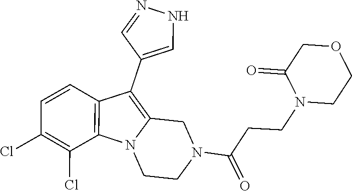 [SMILES: O=C1COCCN1CCC(=O)N1CCn2c(c(-c3cn[nH]c3)c3ccc(Cl)c(Cl)c32)C1] | ES/MS (m/z): (³⁵Cl/³⁷Cl) 462.2/464.2 (M + H) |
| 305 | 5-(6,7-Dichloro-10-(1H- pyrazol-4-yl)-1,2,3,4- tetrahydropyrazino[1,2- a]indole-2-carbonyl)-1- methylpiperidin-2-one | 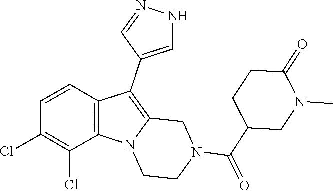 [SMILES: CN1CC(C(=O)N2CCn3c(c(-c4cn[nH]c4)c4ccc(Cl)c(Cl)c43)C2)CCC1=O] | ES/MS (m/z): (³⁵Cl/³⁷Cl) 446.2/448.2 (M + H) |
| 306 | 1-(6,7-Dichloro-10-(1H- pyrazol-4-yl)-3,4- dihydropyrazino[1,2-a]indol- 2(1H)-yl)-2-(2- methoxyethoxy)ethan-1-one | 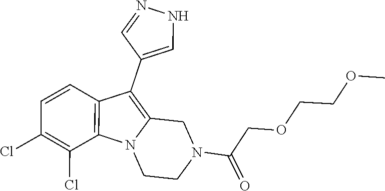 [SMILES: COCCOCC(=O)N1CCn2c(c(-c3cn[nH]c3)c3ccc(Cl)c(Cl)c32)C1] | ES/MS (m/z): (³⁵Cl/³⁷Cl) 423.2/425.2 (M + H) |
| 307 | 3-(2-(6,7-Dichloro-10-(1H- pyrazol-4-yl)-3,4- dihydropyrazino[1,2-a]indol- 2(1H)-yl)-2- oxoethyl)thiazolidine-2,4- dione | 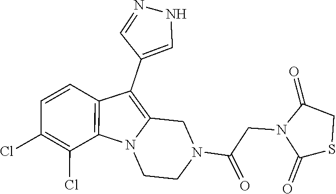 [SMILES: O=C(CN1C(=O)CSC1=O)N1CCn2c(c(-c3cn[nH]c3)c3ccc(Cl)c(Cl)c32)C1] | ES/MS (m/z): (³⁵Cl/³⁷Cl) 464.2/466.2 (M + H) |
| 308 | N-(3-(6,7-Dichloro-10-(1H- pyrazol-4-yl)-3,4- dihydropyrazino[1,2-a]indol- 2(1H)-yl)-3-oxopropyl)-N- methylacetamide | 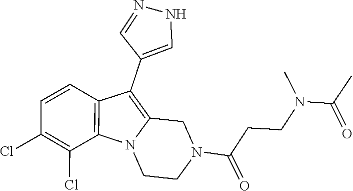 [SMILES: CC(=O)N(C)CCC(=O)N1CCn2c(c(-c3cn[nH]c3)c3ccc(Cl)c(Cl)c32)C1] | ES/MS (m/z): (³⁵Cl/³⁷Cl) 434.2/436.2 (M + H) |
| 309 | (6,7-Dichloro-10-(1H- pyrazol-4-yl)-3,4- dihydropyrazino[1,2-a]indol- 2(1H)-yl)(4- methylmorpholin-3- yl)methanone | 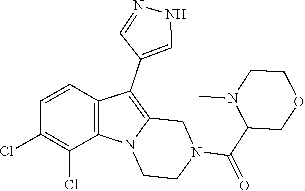 [SMILES: CN1CCOCC1C(=O)N1CCn2c(c(-c3cn[nH]c3)c3ccc(Cl)c(Cl)c32)C1] | ES/MS (m/z): (³⁵Cl/³⁷Cl) 434.2/436.2 (M + H) |
| 310 | (6,7-Dichloro-10-(1H- pyrazol-4-yl)-3,4- dihydropyrazino[1,2-a]indol- 2(1H)-yl)(1,4-dioxepan-6- yl)methanone | 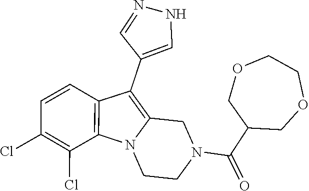 [SMILES: O=C(C1COCCOC1)N1CCn2c(c(-c3cn[nH]c3)c3ccc(Cl)c(Cl)c32)C1] | ES/MS (m/z): (³⁵Cl/³⁷Cl) 435.2/437.2 (M + H) |
| \|311 | 3-(2-(6,7-Dichloro-10-(1H- pyrazol-4-yl)-3,4- dihydropyrazino[1,2-a]indol- 2(1H)-yl)-2-oxoethyl)-1- methylimidazolidine-2,4- dione | 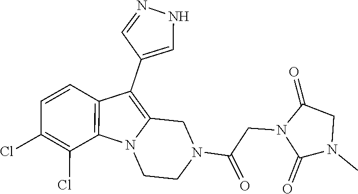 [SMILES: CN1CC(=O)N(CC(=O)N2CCn3c(c(-c4cn[nH]c4)c4ccc(Cl)c(Cl)c43)C2)C1=O] | ES/MS (m/z): (³⁵Cl/³⁷Cl) 461.2/463.2 (M + H) |
| 312 | 4-(6,7-dichloro-10-(1H- pyrazol-4-yl)-1,2,3,4- tetrahydropyrazino[1,2- a]indole-2-carbonyl)-1-(2- methoxyethyl)pyrrolidin-2- one | 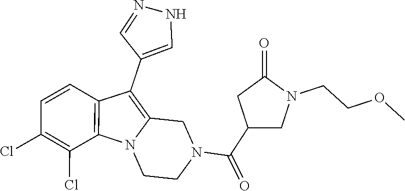 [SMILES: COCCN1CC(C(=O)N2CCn3c(c(-c4cn[nH]c4)c4ccc(Cl)c(Cl)c43)C2)CC1=O] | ES/MS (m/z): (³⁵Cl/³⁷Cl) 476.2/478.2 (M + H) |
| 313 | 1-(2-(6,7-Dichloro-10-(1H- pyrazol-4-yl)-3,4- dihydropyrazino[1,2-a]indol- 2(1H)-yl)-2-oxoethyl)-3- methylimidazolidin-2-one | 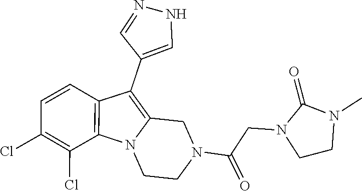 [SMILES: CN1CCN(CC(=O)N2CCn3c(c(-c4cn[nH]c4)c4ccc(Cl)c(Cl)c43)C2)C1=O] | ES/MS (m/z): (³⁵Cl/³⁷Cl) 447.2/449.2 (M + H) |
| 314 | 4-(6,7-Dichloro-10-(1H- pyrazol-4-yl)-1,2,3,4- tetrahydropyrazino[1,2- a]indole-2-carbonyl)-1,3- oxazinan-2-one | 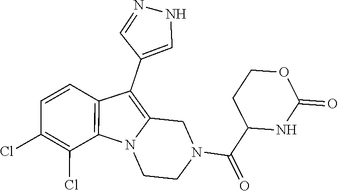 [SMILES: O=C1NC(C(=O)N2CCn3c(c(-c4cn[nH]c4)c4ccc(Cl)c(Cl)c43)C2)CCO1] | ES/MS (m/z): (³⁵Cl/³⁷Cl) 434.2/436.2 (M + H) |
| 315 | 4-(2-(6,7-Dichloro-10-(1H- pyrazol-4-yl)-3,4- dihydropyrazino[1,2-a]indol- 2(1H)-yl)-2- oxoethyl)morpholin-3-one | 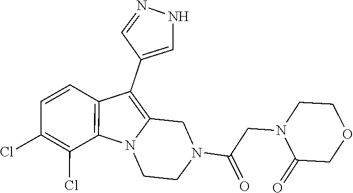 [SMILES: O=C1COCCN1CC(=O)N1CCn2c(c(-c3cn[nH]c3)c3ccc(Cl)c(Cl)c32)C1] | ES/MS (m/z): (³⁵Cl/³⁷Cl) 448.2/450.2 (M + H) |
| 316 | 1-(2-(6,7-Dichloro-10-(1H- pyrazol-4-yl)-3,4- dihydropyrazino[1,2-a]indol- 2(1H)-yl)-2- oxoethyl)pyrrolidin-2-one | 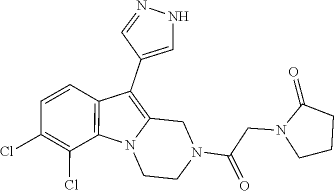 [SMILES: O=C1CCCN1CC(=O)N1CCn2c(c(-c3cn[nH]c3)c3ccc(Cl)c(Cl)c32)C1] | ES/MS (m/z): (³⁵Cl/³⁷Cl) 432.2/434.2 (M + H) |
| 317 | 1-(6,7-Dichloro-10-(1H- pyrazol-4-yl)-3,4- dihydropyrazino[1,2-a]indol- 2(1H)-yl)-3-(2- methoxyethoxy)propan-1- one | 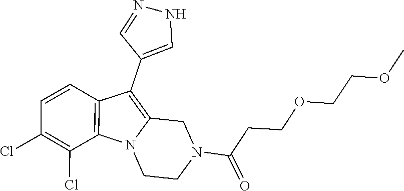 [SMILES: COCCOCCC(=O)N1CCn2c(c(-c3cn[nH]c3)c3ccc(Cl)c(Cl)c32)C1] | ES/MS (m/z): (³⁵Cl/³⁷Cl) 437.2/439.2 (M + H) |
| 318 | N-(2-(6,7-Dichloro-10-(1H- pyrazol-4-yl)-3,4- dihydropyrazino[1,2-a]indol- 2(1H)-yl)-2-oxoethyl)-2- methoxy-N-methylacetamide | 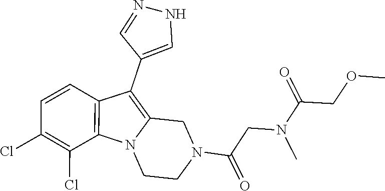 [SMILES: COCC(=O)N(C)CC(=O)N1CCn2c(c(-c3cn[nH]c3)c3ccc(Cl)c(Cl)c32)C1] | ES/MS (m/z): (³⁵Cl/³⁷Cl) 450.2/452.2 (M + H) |
| 319 | 4-(6,7-Dichloro-10-(1H- pyrazol-4-yl)-1,2,3,4- tetrahydropyrazino[1,2- a]indole-2-carbonyl)-1- methylpyrrolidin-2-one | 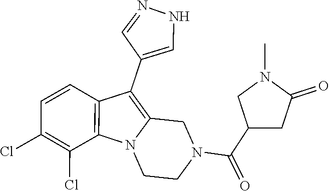 [SMILES: CN1CC(C(=O)N2CCn3c(c(-c4cn[nH]c4)c4ccc(Cl)c(Cl)c43)C2)CC1=O] | ES/MS (m/z): (³⁵Cl/³⁷Cl) 432.2/434.2 (M + H) |
| 320 | 5-(6,7-Dichloro-10-(1H- pyrazol-4-yl)-1,2,3,4- tetrahydropyrazino[1,2- a]indole-2-carbonyl)-1- ethylpyrrolidin-2-one | 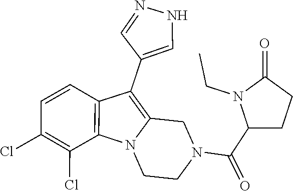 [SMILES: CCN1C(=O)CCC1C(=O)N1CCn2c(c(-c3cn[nH]c3)c3ccc(Cl)c(Cl)c32)C1] | ES/MS (m/z): (³⁵Cl/³⁷Cl) 446.2/448.2 (M + H) |
| 321 | 1-(3-(6,7-Dichloro-10-(1H- pyrazol-4-yl)-1,2,3,4- tetrahydropyrazino[1,2- a]indole-2-carbonyl)azetidin- 1-yl)ethan-1-one | 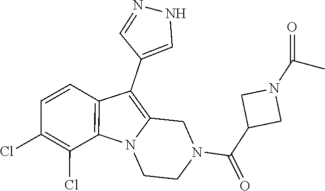 [SMILES: CC(=O)N1CC(C(=O)N2CCn3c(c(-c4cn[nH]c4)c4ccc(Cl)c(Cl)c43)C2)C1] | ES/MS (m/z): (³⁵Cl/³⁷Cl) 432.2/434.4 (M + H) |
| 322 | 1-(6,7-Dichloro-10-(1H- pyrazol-4-yl)-3,4- dihydropyrazino[1,2-a]indol- 2(1H)-yl)-2-((3-methyl- 1,2,4-oxadiazol-5- yl)methoxy)ethan-1-one | 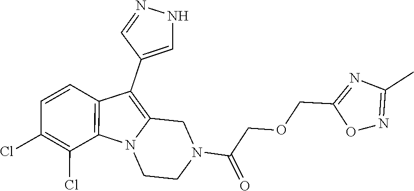 [SMILES: Cc1noc(COCC(=O)N2CCn3c(c(-c4cn[nH]c4)c4ccc(Cl)c(Cl)c43)C2)n1] | ES/MS (m/z): (³⁵Cl/³⁷Cl) 461.2/463.2 (M + H) |
| 323 | 5-(2-(6,7-Dichloro-10-(1H- pyrazol-4-yl)-3,4- dihydropyrazino[1,2-a]indol- 2(1H)-yl)-2-oxoethyl)-3- methylthiazolidine-2,4-dione | 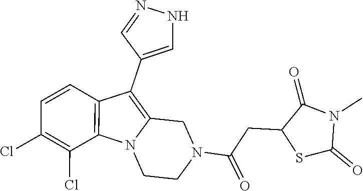 [SMILES: CN1C(=O)SC(CC(=O)N2CCn3c(c(-c4cn[nH]c4)c4ccc(Cl)c(Cl)c43)C2)C1=O] | ES/MS (m/z): (³⁵Cl/³⁷Cl) 478.2/480.2 (M + H) |
| 324 | 5-(2-(6,7-Dichloro-10-(1H- pyrazol-4-yl)-3,4- dihydropyrazino[1,2-a]indol- 2(1H)-yl)-2-oxoethyl)-1- methylpyrrolidin-2-one | 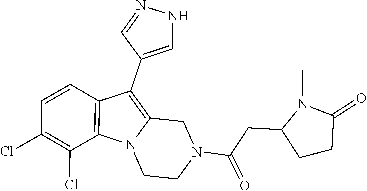 [SMILES: CN1C(=O)CCC1CC(=O)N1CCn2c(c(-c3cn[nH]c3)c3ccc(Cl)c(Cl)c32)C1] | ES/MS (m/z): (³⁵Cl/³⁷Cl) 446.2/448.2 (M + H) |
| 325 | 1-(6,7-Dichloro-10-(1H- pyrazol-4-yl)-3,4- dihydropyrazino[1,2-a]indol- 2(1H)-yl)-3-(5- fluoropyrimidin-2-yl)propan- 1-one | 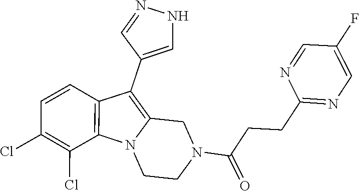 [SMILES: O=C(CCc1ncc(F)cn1)N1CCn2c(c(-c3cn[nH]c3)c3ccc(Cl)c(Cl)c32)C1] | ES/MS (m/z): (³⁵Cl/³⁷Cl) 459.2/461.2 (M + H) |
| 326 | 5-(6,7-Dichloro-10-(1H- pyrazol-4-yl)-1,2,3,4- tetrahydropyrazino[1,2- a]indole-2-carbonyl)-1- methylpyrrolidin-2-one | 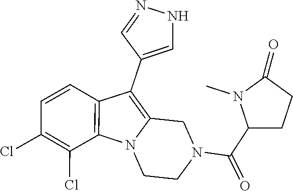 [SMILES: CN1C(=O)CCC1C(=O)N1CCn2c(c(-c3cn[nH]c3)c3ccc(Cl)c(Cl)c32)C1] | ES/MS (m/z): (³⁵Cl/³⁷Cl) 432.2/434.2 (M + H) |
| 327 | 1-(2-(6,7-Dichloro-10-(1H- pyrazol-4-yl)-3,4- dihydropyrazino[1,2-a]indol- 2(1H)-yl)-2-oxoethyl)-3- methylimidazolidine-2,4- dione | 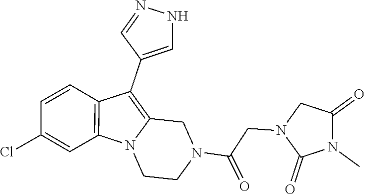 [SMILES: CN1C(=O)CN(CC(=O)N2CCn3c(c(-c4cn[nH]c4)c4ccc(Cl)cc43)C2)C1=O] | ES/MS (m/z): (³⁵Cl/³⁷Cl) 461.2/463.2 (M + H) |
| 328 | 1-(6,7-Dichloro-10-(1H- pyrazol-4-yl)-3,4- dihydropyrazino[1,2-a]indol- 2(1H)-yl)-3-(1H-imidazol-1- yl)propan-1-one | 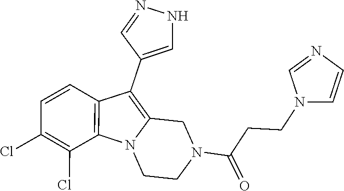 [SMILES: O=C(CCn1ccnc1)N1CCn2c(c(-c3cn[nH]c3)c3ccc(Cl)c(Cl)c32)C1] | ES/MS (m/z): (³⁵Cl/³⁷Cl) 429.2/431.2 (M + H) |
| 329 | N-(1-(6,7-Dichloro-10-(1H- pyrazol-4-yl)-3,4- dihydropyrazino[1,2-a]indol- 2(1H)-yl)-1-oxopropan-2- yl)propionamide | 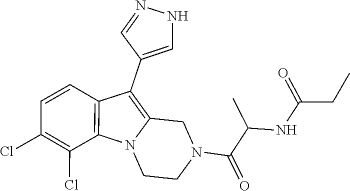 [SMILES: CCC(=O)NC(C)C(=O)N1CCn2c(c(-c3cn[nH]c3)c3ccc(Cl)c(Cl)c32)C1] | ES/MS (m/z): (³⁵Cl/³⁷Cl) 434.2/436.2 (M + H) |
| 330 | 1-(6,7-Dichloro-10-(1H- pyrazol-4-yl)-3,4- dihydropyrazino[1,2-a]indol- 2(1H)-yl)-2-(3- (dimethylamino)oxetan-3- yl)ethan-1-one | 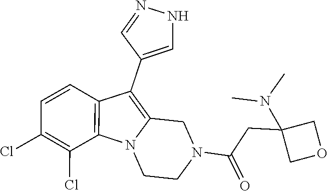 [SMILES: CN(C)C1(CC(=O)N2CCn3c(c(-c4cn[nH]c4)c4ccc(Cl)c(Cl)c43)C2)COC1] | ES/MS (m/z): (³⁵Cl/³⁷Cl) 448.2/450.2 (M + H) |
| 331 | 6-(6,7-Dichloro-10-(1H- pyrazol-4-yl)-1,2,3,4- tetrahydropyrazino[1,2- a]indole-2-carbonyl)-1- methylpiperidin-2-one | 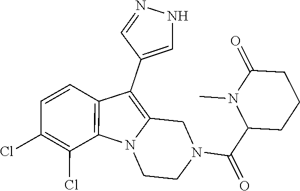 [SMILES: CN1C(=O)CCCC1C(=O)N1CCn2c(c(-c3cn[nH]c3)c3ccc(Cl)c(Cl)c32)C1] | ES/MS (m/z): (³⁵Cl/³⁷Cl) 446.2/448.2 (M + H) |
| 332 | (6,7-Dichloro-10-(1H- pyrazol-4-yl)-3,4- dihydropyrazino[1,2-a]indol- 2(1H)-yl)(3- (dimethylamino)oxetan-3- yl)methanone | 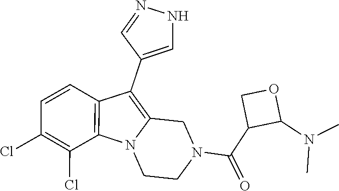 [SMILES: CN(C)C1OCC1C(=O)N1CCn2c(c(-c3cn[nH]c3)c3ccc(Cl)c(Cl)c32)C1] | ES/MS (m/z): (³⁵Cl/³⁷Cl) 434.2/436.2 (M + H) |
| 333 | N-(1-(6,7-Dichloro-10-(1H- pyrazol-4-yl)-3,4- dihydropyrazino[1,2-a]indol- 2(1H)-yl)-1-oxopropan-2-yl)- 2-methoxyacetamide | 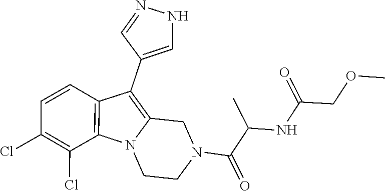 [SMILES: COCC(=O)NC(C)C(=O)N1CCn2c(c(-c3cn[nH]c3)c3ccc(Cl)c(Cl)c32)C1] | ES/MS (m/z): 450.2/452.2 (M + H) |
| 334 | (6,7-Dichloro-10-(1H- pyrazol-4-yl)-3,4- dihydropyrazino[1,2-a]indol- 2(1H)-yl)(3- methoxytetrahydrofuran-3- yl)methanone | 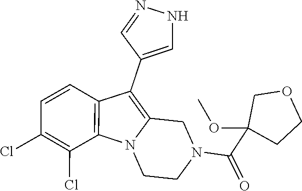 [SMILES: COC1(C(=O)N2CCn3c(c(-c4cn[nH]c4)c4ccc(Cl)c(Cl)c43)C2)CCOC1] | ES/MS (m/z): (³⁵Cl/³⁷Cl) 435.2/437.2 (M + H) |
| 335 | rac-(R)-4-(2-(6,7-Dichloro- 10-(1H-pyrazol-4-yl)-3,4- dihydropyrazino[1,2-a]indol- 2(1H)-yl)-2- oxoethyl)piperidin-2-one | 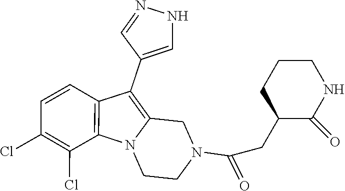 [SMILES: O=C1NCCC[C@@H]1CC(=O)N1CCn2c(c(-c3cn[nH]c3)c3ccc(Cl)c(Cl)c32)C1] | ES/MS (m/z): (³⁵Cl/³⁷Cl) 446.0/448.0 (M + H) |

### Intermediate 648

### tert-Butyl N-[6,7-dichloro-2-tetrahydropyran-2-yl-3-(1-tetrahydropyran-2-ylpyrazol-4-yl)indazol-4-yl]carbamate

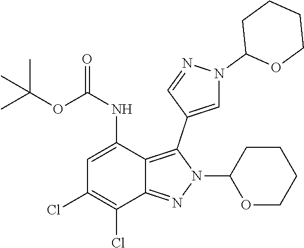 [SMILES: CC(C)(C)OC(=O)Nc1cc(Cl)c(Cl)c2nn(C3CCCCO3)c(-c3cnn(C4CCCCO4)c3)c12]

Suspend 4-bromo-6,7-dichloro-2-tetrahydropyran-2-yl-3-(1-tetrahydropyran-2-ylpyrazol-4-yl) indazole (900 mg, 1.61 mmol), tert-butyl carbamate (400 mg, 3.41 mmol), Pd₂(dba)₃ (150 mg, 0.163 mmol), Xantphos (190 mg, 0.328 mmol), and Cs₂CO₃ (1.3 g, 4.0 mmol) in 1,4-dioxane (40 mL). Degas and purge with N₂ (3×), then stir at 100° C. for 3 hrs., under N₂ atmosphere. Filter and dilute the filtrate with water, extract with EtOAc, dry over anhydrous Na₂SO₄, filter and concentrate under vacuum. Purify by flash silica gel chromatography eluting with EtOAc in petroleum ether to give tert-butyl N-[6,7-dichloro-2-tetrahydropyran-2-yl-3-(1-tetrahydropyran-2-ylpyrazol-4-yl)indazol-4-yl]carbamate (500 mg, 52%) as a white solid. ES/MS (m/z): (³⁵Cl/³⁷Cl) 536.2/538.2 (M+H).

### Intermediate 649

### 6,7-Dichloro-3-(1H-pyrazol-4-yl)-1H-indazol-4-amine

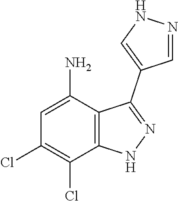 [SMILES: Nc1cc(Cl)c(Cl)c2[nH]nc(-c3cn[nH]c3)c12]

Dissolve tert-butyl N-[6,7-dichloro-2-tetrahydropyran-2-yl-3-(1-tetrahydropyran-2-ylpyrazol-4-yl)indazol-4-yl]carbamate (500 mg, 0.834 mmol) in 4M HCl in MeOH (20 mL). Stir at ambient temperature for 4 hrs. Filter and dry under vacuum to give 6,7-dichloro-3-(1H-pyrazol-4-yl)-1H-indazol-4-amine (200 mg, 90%, HCl salt) as a brown solid. ES/MS (m/z): (³⁵Cl/³⁷Cl) 269.1/271.1 (M+H).

### Intermediate 650

### 5,6-Dichloro-9-(1H-pyrazol-4-yl)-1H-pyrrolo[1,2-a]indol-2(3H)-one

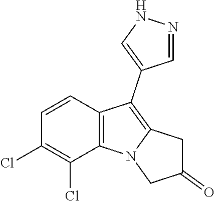 [SMILES: O=C1Cc2c(-c3cn[nH]c3)c3ccc(Cl)c(Cl)c3n2C1]

Dissolve 5,6-dichloro-9-(1-(tetrahydro-2H-pyran-2-yl)-1H-pyrazol-4-yl)-1H-pyrrolo[1,2-a]indol-2(3H)-one (80 mg, 0.20 mmol) in DCM (2 mL) and add TFA (250 μL, 3.31 mmol). Stir at ambient temperature for 6 hrs and add TFA (100 μL, 1.32 mmol). Stir at ambient temperature for 1.5 hrs, add a few drops of 2-propanol, and stir at ambient temperature for 4 hrs. Concentrate under vacuum and purify by reverse phase chromatography to give 5,6-dichloro-9-(1H-pyrazol-4-yl)-1H-pyrrolo[1,2-a]indol-2(3H)-one (36.7 mg, 58%). ES/MS (m/z): (³⁵Cl/³⁷Cl) 306.0/308.0 (M+H).

### EXAMPLE 336

### N-(6, 7-Dichloro-3-(1H-pyrazol-4-yl)-1H-indazol-4-yl)-2-hydroxyacetamide

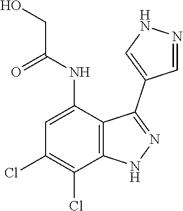 [SMILES: O=C(CO)Nc1cc(Cl)c(Cl)c2[nH]nc(-c3cn[nH]c3)c12]

Dissolve 6,7-dichloro-3-(1H-pyrazol-4-yl)-1H-indazol-4-amine (200 mg, 0.671 mmol) and TEA (0.6 mL, 4 mmol) in DCM (10 mL) and stir at ambient temperature for 0.5 hr. Add (2-chloro-2-oxo-ethyl) acetate (400 mg, 2.93 mmol) and stir at ambient temperature for 16-18 hrs. Quench with water, extract with EtOAc, filter, and concentrate under vacuum. Dissolve the residue in MeOH (10 mL) and add potassium carbonate (100 mg, 0.724 mmol), then stir at ambient temperature for 4 hrs. Concentrate under vacuum and purify by reverse phase prep-HPLC to give N-(6,7-dichloro-3-(1H-pyrazol-4-yl)-1H-indazol-4-yl)-2-hydroxyacetamide (59.82 mg, 27%) as a white solid. ES/MS (m/z): (³⁵Cl/³⁷Cl) 326.2/328.2 (M+H).

### EXAMPLE 337

### N-(6,7-Dichloro-3-(1H-pyrazol-4-yl)-1H-indazol-4-yl)-2,2-difluoroacetamide

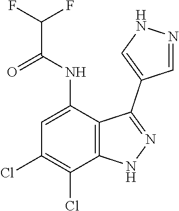 [SMILES: O=C(Nc1cc(Cl)c(Cl)c2[nH]nc(-c3cn[nH]c3)c12)C(F)F]

Dissolve 6,7-dichloro-3-(1H-pyrazol-4-yl)-1H-indazol-4-amine (200 mg, 0.671 mmol) and ethyl difluoroacetate (0.3 mL, 3 mmol) in DCM (5 mL) and add Me₃Al (2 mL, 4.0 mmol, 2 M in toluene) under N₂ atmosphere. Stir at ambient temperature for 16-18 hrs. Quench with saturated aqueous sodium potassium tartrate, extract with EtOAc, wash with saturated aqueous NaCl, dry over anhydrous Na₂SO₄, filter, and concentrate under vacuum. Purify by reverse phase prep-HPLC to give N-(6,7-dichloro-3-(1H-pyrazol-4-yl)-1H-indazol-4-yl)-2,2-difluoroacetamide (114 mg, 48%) as a white solid. ES/MS (m/z): (³⁵Cl/³⁷Cl) 346.2/348.2 (M+H).

### Intermediate 651

### 4-(3-((tert-Butyldimethylsilyl)oxy)propoxy)-6,7-dichloro-2-(((tetrahydro-2H-pyran-2-yl)oxy)methyl)-1H-indole

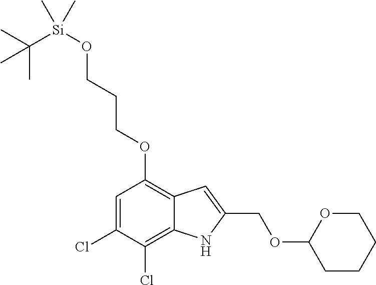 [SMILES: CC(C)(C)[Si](C)(C)OCCCOc1cc(Cl)c(Cl)c2[nH]c(COC3CCCCO3)cc12]

Combine 6,7-dichloro-2-(tetrahydropyran-2-yloxymethyl)-1H-indol-4-ol (500 mg, 1.34 mmol) and potassium carbonate (223 mg, 1.61 mmol) in DMF (10 mL). Cool to 0° C. and add tert-butyl(3-iodopropoxy)dimethylsilane (807 mg, 2.69 mmol). Stir at RT for 6 hrs. Dilute with water, extract with EtOAc (3×), wash the combined organic layers with saturated aqueous NaCl, dry over anhydrous sodium sulfate, filter, and concentrate under vacuum. Purify by flash column chromatography eluting with EtOAc in petroleum ether to give 4-(3-((tert-butyldimethylsilyl)oxy)propoxy)-6,7-dichloro-2-(((tetrahydro-2H-pyran-2-yl)oxy)methyl)-1H-indole (550 mg, 75%) as a yellow gum. ES/MS (m/z): (³⁵Cl/³⁷Cl) 386.0/387.7 (M+H-THP-H₂O) and 487.2/488.9 (M−H).

### Intermediate 652

### 4-(3-((tert-Butyldimethylsilyl)oxy)propoxy)-6,7-dichloro-3-iodo-2-(((tetrahydro-2H-pyran-2-yl)oxy)methyl)-1H-indole

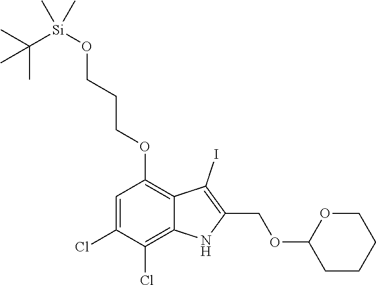 [SMILES: CC(C)(C)[Si](C)(C)OCCCOc1cc(Cl)c(Cl)c2[nH]c(COC3CCCCO3)c(I)c12]

Dissolve 4-(3-((tert-butyldimethylsilyl)oxy)propoxy)-6,7-dichloro-2-(((tetrahydro-2H-pyran-2-yl)oxy)methyl)-1H-indole (250 mg, 0.461 mmol) in DMF (5 mL) and add NIS (124 mg, 0.553 mmol) at 0° C. Stir at RT for 1 hr. Quench with saturated aqueous Na₂SO₃ and extract with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give 4-(3-((tert-butyldimethylsilyl)oxy)propoxy)-6,7-dichloro-3-iodo-2-(((tetrahydro-2H-pyran-2-yl)oxy)methyl)-1H-indole (270 mg, 89%) as a yellow gum. ES/MS (m/z): (³⁵Cl/³⁷Cl) 487.2/489.1 (M−H−I) and 636.8 (M+Na, small peak).

### Intermediate 653

### 4-(3-((tert-Butyldimethylsilyl)oxy)propoxy)-6,7-dichloro-3-(1-(tetrahydro-2H-pyran-2-yl)-1H-pyrazol-4-yl)-2-(((tetrahydro-2H-pyran-2-yl)oxy)methyl)-1H-indole

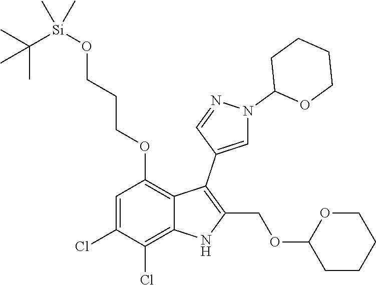 [SMILES: CC(C)(C)[Si](C)(C)OCCCOc1cc(Cl)c(Cl)c2[nH]c(COC3CCCCO3)c(-c3cnn(C4CCCCO4)c3)c12]

Combine 4-(3-((tert-butyldimethylsilyl)oxy)propoxy)-6,7-dichloro-3-iodo-2-(((tetrahydro-2H-pyran-2-yl)oxy)methyl)-1H-indole (270 mg, 0.409 mmol), 1-tetrahydropyran-2-yl-4-(4,4,5,5-tetramethyl-1,3,2-dioxaborolan-2-yl)pyrazole (125 mg, 0.450 mmol), sodium carbonate (130 mg, 1.23 mmol), and Pd(dtbpf)Cl₂ (53 mg, 0.082 mmol) in 1,4-dioxane (12 mL) and water (3.0 mL). Purge with N₂ and stir at 90° C. for 2 hrs. Concentrate and purify by flash column chromatography eluting with EtOAc in petroleum ether to give 4-(3-((tert-butyldimethylsilyl)oxy)propoxy)-6,7-dichloro-3-(1-(tetrahydro-2H-pyran-2-yl)-1H-pyrazol-4-yl)-2-(((tetrahydro-2H-pyran-2-yl)oxy)methyl)-1H-indole (200 mg, 67%) as a yellow oil. ES/MS (m/z): (³⁵Cl/³⁷Cl) 638.3/640.4 (M+H) and 660.4/662.2 (M+Na).

### EXAMPLE 338

### 3-((6,7-Dichloro-2-(hydroxymethyl)-3-(1H-pyrazol-4-yl)-1H-indol-4-yl)oxy)propan-1-ol

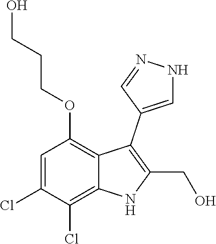 [SMILES: OCCCOc1cc(Cl)c(Cl)c2[nH]c(CO)c(-c3cn[nH]c3)c12]

Dissolve 4-(3-((tert-butyldimethylsilyl)oxy)propoxy)-6,7-dichloro-3-(1-(tetrahydro-2H-pyran-2-yl)-1H-pyrazol-4-yl)-2-(((tetrahydro-2H-pyran-2-yl)oxy)methyl)-1H-indole (200 mg, 0.276 mmol) in THF (4 mL). Add 6M aqueous HCl (4 mL) and stir at RT for 4 hrs. Pour into 2M aqueous LiOH (20 mL) at 0° C. and extract with EtOAc (3×). Wash the combined organic layers with saturated aqueous NaCl, dry over anhydrous sodium sulfate, filter, and concentrate under vacuum. Purify by prep-HPLC to give 3-((6, 7-dichloro-2-(hydroxymethyl)-3-(1H-pyrazol-4-yl)-1H-indol-4-yl)oxy)propan-1-ol (25.73 mg, 26%) as a white solid. ES/MS (m/z): (³⁵Cl/³⁷Cl) 356.0/358.0 (M+H).

### Intermediate 654

### 9-(3-((tert-Butyldimethylsilyl)oxy)propoxy)-6,7-dichloro-2-methyl-3,4-dihydropyrazino[1,2-a]indol-1(2H)-one

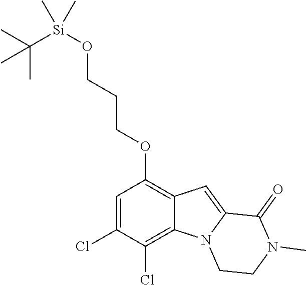 [SMILES: CN1CCn2c(cc3c(OCCCO[Si](C)(C)C(C)(C)C)cc(Cl)c(Cl)c32)C1=O]

Combine 6,7-dichloro-9-hydroxy-2-methyl-3,4-dihydropyrazino[1,2-a]indol-1(2H)-one (400 mg, 0.856 mmol) and potassium carbonate (149 mg, 1.03 mmol) in DMF (8 mL). Cool to 0° C. and add tert-butyl(3-iodopropoxy)dimethylsilane (406 mg, 1.28 mmol). Stir at RT for 6 hrs. Dilute with water, extract with EtOAc (2×), wash the combined organic layers with saturated aqueous NaCl, dry over anhydrous sodium sulfate, filter, and concentrate under vacuum. Purify by flash column chromatography eluting with EtOAc in petroleum ether to give 9-(3-((tert-butyldimethylsilyl)oxy)propoxy)-6,7-dichloro-2-methyl-3,4-dihydropyrazino[1,2-a]indol-1(2H)-one (460 mg, 99+%) as a light-yellow solid. ES/MS (m/z): (³⁵Cl/³⁷Cl) 457.2/459.2 (M+H).

### Intermediate 655

### 10-Bromo-9-(3-((tert-butyldimethylsilyl)oxy)propoxy)-6,7-dichloro-2-methyl-3,4-dihydropyrazino[1,2-a]indol-1(2H)-one

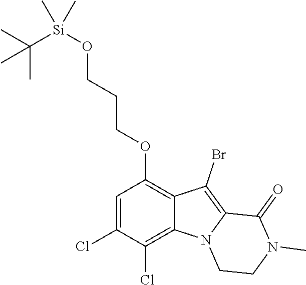 [SMILES: CN1CCn2c(c(Br)c3c(OCCCO[Si](C)(C)C(C)(C)C)cc(Cl)c(Cl)c32)C1=O]

Dissolve 9-(3-((tert-butyldimethylsilyl)oxy)propoxy)-6,7-dichloro-2-methyl-3,4-dihydropyrazino[1,2-a]indol-1(2H)-one (460 mg, 0.855 mmol) in DMF (10 mL) and add NBS (200 mg, 1.07 mmol) at 0° C. Stir at RT for 2 hrs. Quench with saturated aqueous Na₂SO₃ and extract with EtOAc (2×). Wash the combined organic layers with saturated aqueous NaCl (3×), dry over anhydrous sodium sulfate, filter, and concentrate under vacuum. Purify by flash column chromatography eluting with EtOAc in petroleum ether to give 10-bromo-9-(3-((tert-butyldimethylsilyl)oxy)propoxy)-6,7-dichloro-2-methyl-3,4-dihydropyrazino[1,2-a]indol-1(2H)-one (450 mg, 84%) as a light-yellow solid. ES/MS (m/z): 535.2/537.2/539.2 (M+H).

### Intermediate 656

### 9-(3-((tert-Butyldimethylsilyl)oxy)propoxy)-6,7-dichloro-2-methyl-10-(1-(tetrahydro-2H-pyran-2-yl)-1H-pyrazol-4-yl)-3,4-dihydropyrazino[1,2-a]indol-1(2H)-one

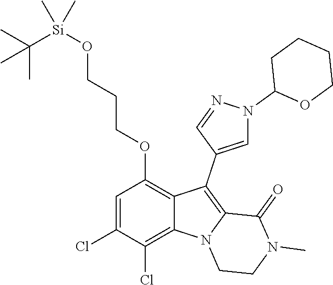 [SMILES: CN1CCn2c(c(-c3cnn(C4CCCCO4)c3)c3c(OCCCO[Si](C)(C)C(C)(C)C)cc(Cl)c(Cl)c32)C1=O]

Combine 10-bromo-9-(3-((tert-butyl dimethyl silyl)oxy)propoxy)-6, 7-dichloro-2-methyl-3,4-dihydropyrazino[1,2-a]indol-1(2H)-one (250 mg, 0.401 mmol), 1-tetrahydropyran-2-yl-4-(4,4,5,5-tetramethyl-1,3,2-dioxaborolan-2-yl)pyrazole (171 mg, 0.601 mmol), sodium carbonate (87 mg, 0.80 mmol), and Pd(dtbpf)Cl₂ (30 mg, 0.040 mmol) in 1,4-dioxane (16 mL) and water (4.0 mL). Purge with N₂ and stir at 90° C. for 2 hrs. Combine with another batch run at 0.80×scale for work up and purification. Dilute with water, extract with EtOAc (2×), wash the combined organic layers with saturated aqueous NaCl (2×), dry over anhydrous sodium sulfate, filter, and concentrate under vacuum. Purify by flash column chromatography eluting with EtOAc in petroleum ether to give 9-(3-((tert-butyldimethylsilyl)oxy)propoxy)-6,7-dichloro-2-methyl-10-(1-(tetrahydro-2H-pyran-2-yl)-1H-pyrazol-4-yl)-3,4-dihydropyrazino[1,2-a]indol-1(2H)-one (350 mg, 69%) as a brown oil. ES/MS (m/z): (³⁵Cl/³⁷Cl) 607.3/609.3 (M+H).

### EXAMPLE 339

### 6,7-Dichloro-9-(3-hydroxypropoxy)-2-methyl-10-(1H-pyrazol-4-yl)-3,4-dihydropyrazino[1,2-a]indol-1(2H)-one

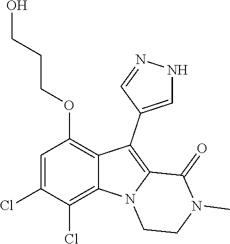 [SMILES: CN1CCn2c(c(-c3cn[nH]c3)c3c(OCCCO)cc(Cl)c(Cl)c32)C1=O]

Dissolve 9-(3-((tert-butyldimethylsilyl)oxy)propoxy)-6,7-dichloro-2-methyl-10-(1-(tetrahydro-2H-pyran-2-yl)-1H-pyrazol-4-yl)-3,4-dihydropyrazino[1,2-a]indol-1(2H)-one (300 mg, 0.430 mmol) in THF (3.0 mL). Add 6M aqueous HCl (3.0 mL) and stir at RT for 1 hr. Combine with another batch run at 0.17×scale for work up and purification. Quench with saturated aqueous sodium bicarbonate and extract with EtOAc (2×). Wash the combined organic layers with saturated aqueous NaCl (2×), dry over anhydrous sodium sulfate, filter, and concentrate under vacuum. Purify by prep-HPLC to give 6,7-dichloro-9-(3-hydroxypropoxy)-2-methyl-10-(1H-pyrazol-4-yl)-3,4-dihydropyrazino[1,2-a]indol-1(2H)-one (112.30 mg, 54%) as a white solid. ES/MS (m/z): (³⁵Cl/³⁷Cl) 409.0/411.0 (M+H).

### Intermediate 657

### 2-((6,7-Dichloro-2-(((tetrahydro-2H-pyran-2-yl)oxy)methyl)-1H-indol-4-yl)oxy)acetonitrile

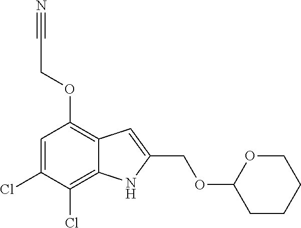 [SMILES: N#CCOc1cc(Cl)c(Cl)c2[nH]c(COC3CCCCO3)cc12]

Combine 6,7-dichloro-2-(tetrahydropyran-2-yloxymethyl)-1H-indol-4-ol (500 mg, 1.34 mmol) and potassium carbonate (223 mg, 1.61 mmol) in DMF (10 mL). Add 2-chloroacetonitrile (807 mg, 2.69 mmol), and stir at RT for 6 hrs. Dilute with water, extract with EtOAc (3×), wash the combined organic layers with saturated aqueous NaCl, dry over anhydrous sodium sulfate, filter, and concentrate under vacuum. Purify by flash column chromatography eluting with EtOAc in petroleum ether to give 2-((6,7-dichloro-2-(((tetrahydro-2H-pyran-2-yl)oxy)methyl)-1H-indol-4-yl)oxy)acetonitrile (350 mg, 70%) as a yellow gum. ES/MS (m/z): (³⁵Cl/³⁷Cl) 252.8/254.8 (M+H-THP-H₂O) and 376.9/378.8 (M+Na).

### Intermediate 658

### 2-((6,7-Dichloro-3-iodo-2-(((tetrahydro-2H-pyran-2-yl)oxy)methyl)-1H-indol-4-yl)oxy)acetonitrile

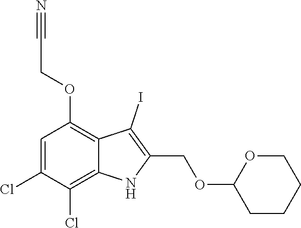 [SMILES: N#CCOc1cc(Cl)c(Cl)c2[nH]c(COC3CCCCO3)c(I)c12]

Dissolve 2-((6,7-dichloro-2-(((tetrahydro-2H-pyran-2-yl)oxy)methyl)-1H-indol-4-yl)oxy)acetonitrile (250 mg, 0.669 mmol) in DMF (5 mL) and add NIS (181 mg, 0.802 mmol) at 0° C. Stir at RT for 1 hr. Quench with saturated aqueous Na₂SO₃ and extract with EtOAc (3×). Wash the combined organic layers with saturated aqueous NaCl, dry over anhydrous sodium sulfate, filter, and concentrate under vacuum. Purify by flash column chromatography eluting with EtOAc in petroleum ether to give 2-((6,7-dichloro-3-iodo-2-(((tetrahydro-2H-pyran-2-yl)oxy)methyl)-1H-indol-4-yl)oxy)acetonitrile (300 mg, 84%) as a yellow gum. ES/MS (m/z): (³⁵Cl/³⁷Cl) 378.8.0/380.8 (M+H-THP-H₂O) and 502.8/504.8 (M+Na).

### Intermediate 659

### 2-((6, 7-Dichloro-3-(1-(tetrahydro-2H-pyran-2-yl)-1H-pyrazol-4-yl)-2-(((tetrahydro-2H-pyran-2-yl)oxy)methyl)-1H-indol-4-yl)oxy)acetonitrile

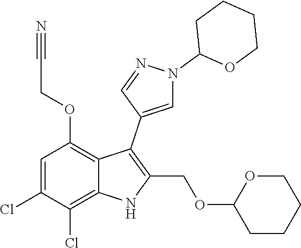 [SMILES: N#CCOc1cc(Cl)c(Cl)c2[nH]c(COC3CCCCO3)c(-c3cnn(C4CCCCO4)c3)c12]

Combine 2-((6,7-dichloro-3-iodo-2-(((tetrahydro-2H-pyran-2-yl)oxy)methyl)-1H-indol-4-yl)oxy)acetonitrile (300 mg, 0.561 mmol), 1-tetrahydropyran-2-yl-4-(4,4,5,5-tetramethyl-1,3,2-dioxaborolan-2-yl)pyrazole (172 mg, 0.617 mmol), sodium carbonate (178 mg, 1.68 mmol), and Pd(dtbpf)Cl₂ (73 mg, 0.11 mmol) in 1,4-dioxane (12 mL) and water (3.0 mL). Purge with N₂ and stir at 90° C. for 2 hrs. Concentrate and purify by flash column chromatography eluting with EtOAc in petroleum ether to give 2-((6,7-dichloro-3-(1-(tetrahydro-2H-pyran-2-yl)-1H-pyrazol-4-yl)-2-(((tetrahydro-2H-pyran-2-yl)oxy)methyl)-1H-indol-4-yl)oxy)acetonitrile (175 mg, 56%) as a yellow oil. ES/MS (m/z): (³⁵Cl/³⁷Cl) 505.0/507.0 (M+H).

### EXAMPLE 340

### 2-((6,7-Dichloro-2-(hydroxymethyl)-3-(1H-pyrazol-4-yl)-1H-indol-4-yl)oxy)acetonitrile

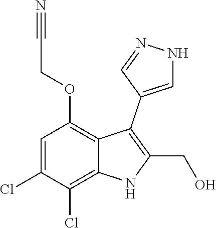 [SMILES: N#CCOc1cc(Cl)c(Cl)c2[nH]c(CO)c(-c3cn[nH]c3)c12]

Dissolve 2-((6,7-dichloro-3-(1-(tetrahydro-2H-pyran-2-yl)-1H-pyrazol-4-yl)-2-(((tetrahydro-2H-pyran-2-yl)oxy)methyl)-1H-indol-4-yl)oxy)acetonitrile (175 mg, 0.312 mmol) in THF (3 mL). Add 6M aqueous HCl (3 mL) and stir at RT for 4 hrs. Combine with another batch run at 0.42×scale for work up and purification. Pour into 2M aqueous LiOH (20 mL) at 0° C. and extract with EtOAc (3×). Wash the combined organic layers with saturated aqueous NaCl, dry over anhydrous sodium sulfate, filter, and concentrate under vacuum. Purify by prep-HPLC to give 2-((6,7-dichloro-2-(hydroxymethyl)-3-(1H-pyrazol-4-yl)-1H-indol-4-yl)oxy)acetonitrile (20.31 mg, 14%) as a white solid. ES/MS (m/z): (³⁵Cl/³⁷Cl) 337.0/338.9 (M+H).

### Intermediate 660

### 6-Chloro-3-iodo-5-methoxy-1H-indole

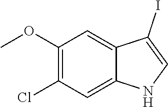

Dissolve 6-chloro-5-methoxy-1H-indole (CAS 63762-72-1, 1.80 g, 9.91 mmol) in DMF (66 mL). Add NIS (2.34 g, 10.4 mmol) and stir at RT for 30 minutes. Dilute with EtOAc and extract with saturated aqueous sodium bicarbonate. Wash with water and saturated aqueous NaCl, dry over anhydrous sodium sulfate, filter and concentrate. Suspend in DCM, collect the solid by filtration and purify the filtrate by flash column chromatography eluting with acetone in hexanes. Combine the material collected by filtration and the material purified by column chromatography. Slurry in hexanes, collect the solid by filtration and rinse with hexanes to give 6-chloro-3-iodo-5-methoxy-1H-indole (2.61 g, 85%) as a brown solid. ES/MS (m/z): (³⁵Cl/³⁷Cl) 305.6/307.6 (M−H, negative ionization mode).

### Intermediate 661

### 6-Chloro-5-methoxy-3-(1-(tetrahydro-2H-pyran-2-yl)-1H-pyrazol-4-yl)-1H-indole

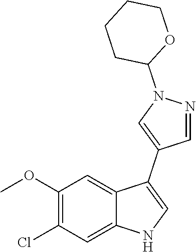 [SMILES: COc1cc2c(-c3cnn(C4CCCCO4)c3)c[nH]c2cc1Cl]

Dissolve 6-chloro-3-iodo-5-methoxy-1H-indole (2.61 g, 8.49 mmol) in 1,4-dioxane (50 mL) and water (10 mL). Add 1,1′-bis(di-tert-butylphosphino)ferrocene palladium dichloride (0.83 g, 0.27 mmol), 1-(tetrahydro-2H-pyran-2-yl)-4-(4,4,5,5-tetramethyl-1,3,2-dioxaboran-2-yl)-1H-pyrazole (5.90 g, 21.2 mmol), and sodium carbonate (2.70 g, 25.5 mmol). Sparge with N₂ for 5 min, then heat to 90° C. under N₂ for 2 hours. Cool to ambient temperature. Dilute with EtOAc and extract with 1:1 water/aqueous saturated sodium bicarbonate. Wash sequentially with 1:1 water/saturated aqueous NaCl and saturated aqueous NaCl, dry over anhydrous sodium sulfate, filter, and concentrate. Purify by column chromatography eluting with acetone in hexanes to give 6-chloro-5-methoxy-3-(1-(tetrahydro-2H-pyran-2-yl)-1H-pyrazol-4-yl)-1H-indole (2.10 g, 93%). ES/MS (m/z): (³⁵Cl/³⁷Cl) 332.2/334.2 (M+H).

### Intermediate 662

### 6-Chloro-5-methoxy-1-(pyridazine-3-yl)-3-(1-(tetrahydro-2H-pyran-2-yl)-1H-pyrazol-4-yl)-1H-indole

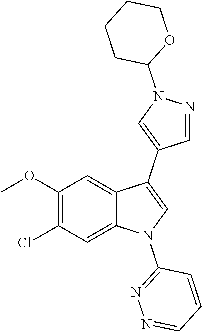 [SMILES: COc1cc2c(-c3cnn(C4CCCCO4)c3)cn(-c3cccnn3)c2cc1Cl]

Dissolve 6-chloro-5-methoxy-3-(1-(tetrahydro-2H-pyran-2-yl)-1H-pyrazol-4-yl)-1H-indole (100 mg, 0.30 mmol) and 3-bromopyridazine (58 mg, 0.36 mmol) in anhydrous NMP (3.0 mL) in a microwave vial. Add cesium carbonate (150 mg, 0.45 mmol) and heat to 130° C. for 4 hrs. Cool to ambient temperature. Dilute with EtOAc, wash sequentially with water and saturated aqueous NaCl, dry over anhydrous sodium sulfate, filter, and concentrate under vacuum. Purify by column chromatography eluting with acetone in hexanes to give 6-chloro-5-methoxy-1-(pyridazine-3-yl)-3-(1-(tetrahydro-2H-pyran-2-yl)-1H-pyrazol-4-yl)-1H-indole (120 mg, 97%) as a brown oil. ES/MS (m/z): (³⁵Cl/³⁷Cl) 409.8/411.8 (M+H).

### EXAMPLE 341

### 6-Chloro-5-methoxy-3-(1H-pyrazol-4-yl)-1-(pyridazin-3-yl)-1H-indole

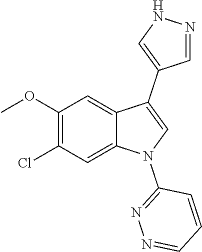 [SMILES: COc1cc2c(-c3cn[nH]c3)cn(-c3cccnn3)c2cc1Cl]

Dissolve 6-chloro-5-methoxy-1-(pyridazine-3-yl)-3-(1-(tetrahydro-2H-pyran-2-yl)-1H-pyrazol-4-yl)-1H-indole (0.12 g, 0.29 mmol) in 1,4-dioxane (4.0 mL) and MeOH (1.0 mL). Add HCl (4.0 N in 1,4-dioxane, 1.1 mL, 4.4 mmol) and stir at ambient temperature in a sealed vial for 16-18 hours. Dilute with EtOAc, wash sequentially with 1:1 water/aqueous saturated sodium bicarbonate, saturated sodium bicarbonate and saturated aqueous NaCl. Dry over anhydrous sodium sulfate, filter and concentrate under vacuum. Suspend material in 75% DCM in hexanes, collect the solid by filtration rinsing with 75% DCM/hexanes. Dry the resulting solid in a vacuum at 35° C. to give 6-chloro-5-methoxy-3-(1H-pyrazol-4-yl)-1-(pyridazin-3-yl)-1H-indole (45 mg, 47%) as a tan solid. ES/MS (m/z): (³⁵Cl/³⁷Cl) 326.0/328.0 (M+H).

### Intermediate 663

### 6-(6-Chloro-5-methoxy -3-(1-(tetrahydro-2H-pyran-2-yl)-1H-pyrazol-4-yl)-1H-indol-1-yl)pyridazine-3-carboxylic acid

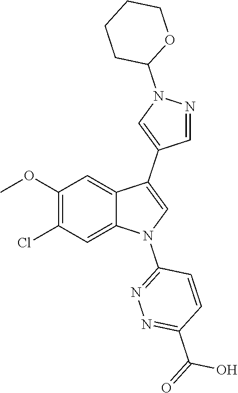 [SMILES: COc1cc2c(-c3cnn(C4CCCCO4)c3)cn(-c3ccc(C(=O)O)nn3)c2cc1Cl]

Dissolve 6-chloro-5-methoxy-3-(1-(tetrahydro-2H-pyran-2-yl)-1H-pyrazol-4-yl)-1H-indole (0.10 mg, 0.30 mmol) and methyl 6-bromopyridazine-3-carboxylate (78 mg, 0.36 mmol) in anhydrous NMP (3.0 mL) in a microwave vial. Add cesium carbonate (150 mg, 0.45 mmol) and heat to 130° C. for 18 hours. Cool to ambient temperature. Dilute with MTBE and add water. Separate the layers and wash the aqueous layer with addition MTBE (2×). Acidify the aqueous layer with aqueous HCl to ˜pH 2, then extract with EtOAc (3×). Wash the combined EtOAc extracts with saturated aqueous NaCl, dry over anhydrous sodium sulfate, filter, and concentrate to give 6-(6-chloro-5-methoxy-3-(1-(tetrahydro-2H-pyran-2-yl)-1H-pyrazol-4-yl)-1H-indol-1-yl)pyridazine-3-carboxylic acid (140 mg, 100%) as a brown oil. Material was used crude in the next step. ES/MS (m/z): (³⁵Cl/³⁷Cl) 454.2/456.2 (M+H).

### Intermediate 664

### (6-(6-Chloro-5-methoxy-3-(1-(tetrahydro-2H-pyran-2-yl)-1H-pyrazol-4-yl)-1H-indol -1-yl)pyridazin-3-yl)methanol

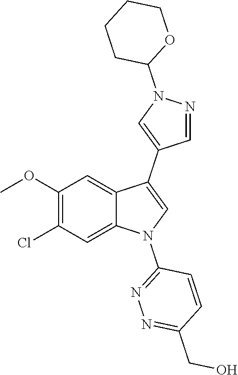 [SMILES: COc1cc2c(-c3cnn(C4CCCCO4)c3)cn(-c3ccc(CO)nn3)c2cc1Cl]

Dissolve 6-(6-chloro-5-methoxy-3-(1-(tetrahydro-2H-pyran-2-yl)-1H-pyrazol-4-yl)-1H-indol-1-yl)pyridazine-3-carboxylic acid (140 mg, 0.31 mmol) in anhydrous THF (5 mL). Add Et₃N (52 μL, 0.37 mmol) followed by isobutyl chloroformate (49 μL, 0.37 mmol) and stir at ambient temperature for 3 hrs. Add NaBH₄ (26 mg, 0.69 mmol) and stir at ambient temperature for 1 hr. Add additional NaBH₄ (26 mg, 0.69 mmol) and stir at ambient temperature for 1.5 hrs. Quench the reaction with aqueous saturated sodium bicarbonate. Dilute with EtOAc, wash sequentially with saturated sodium bicarbonate and saturated aqueous NaCl, dry over anhydrous sodium sulfate, filter, and concentrate under vacuum. Purify by column chromatography eluting with acetone in hexanes to give (6-(6-chloro-5-methoxy-3-(1-(tetrahydro-2H-pyran-2-yl)-1H-pyrazol-4-yl)-1H-indol-1-yl)pyridazin-3-yl)methanol (32 mg, 24%) as a brown oil. ES/MS (m/z): (³⁵Cl/³⁷Cl) 440.0/442.0 (M+H).

### EXAMPLE 342

### (6-(6-chloro-5-methoxy-3-(1H-pyrazol-4-yl)-1H-indol-1-yl)pyridazin-3-yl)methanol

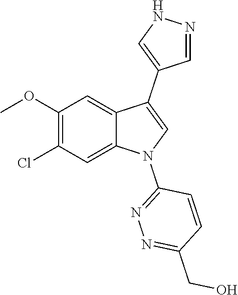 [SMILES: COc1cc2c(-c3cn[nH]c3)cn(-c3ccc(CO)nn3)c2cc1Cl]

Dissolve (6-(6-chloro-5-methoxy-3-(1-(tetrahydro-2H-pyran-2-yl)-1H-pyrazol-4-yl)-1H-indol-1-yl)pyridazin-3-yl)methanol (32 mg, 0.073 mmol) in DCM (3 mL). Add TFA (1 mL, 10 mmol) and stir at ambient temperature for 2.5 hrs. Dilute with EtOAc, wash sequentially with 1:1 water/aqueous saturated sodium bicarbonate, saturated sodium bicarbonate and saturated aqueous NaCl, dry over anhydrous sodium sulfate, filter, and concentrate under vacuum. Purify by column chromatography eluting with acetone in hexanes to give (6-(6-chloro-5-methoxy-3-(1H-pyrazol-4-yl)-1H-indol-1-yl)pyridazin-3-yl)methanol (9 mg, 30%) as a tan solid. ES/MS (m/z): (³⁵Cl/³⁷Cl) 356.2/358.2 (M+H).

### Intermediate 665

### (R)—N—((S)-5,6-Dichloro-8-hydroxy-2,3-dihydro-1H-pyrrolo[1,2-a]indol-1-yl)-2-methylpropane-2-sulfinamide

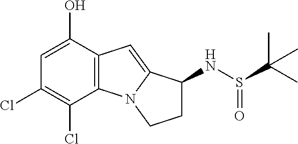 [SMILES: CC(C)(C)[S@@](=O)N[C@H]1CCn2c1cc1c(O)cc(Cl)c(Cl)c12]

Add sodium tert-butoxide (320 mg, 3.3 mmol) and H₂O (2 mL) to (R)—N—((S)-8-bromo-5,6-dichloro-2,3-dihydro-1H-pyrrolo[1,2-a]indol-1-yl)-2-methylpropane-2-sulfinamide (570 mg, 1.3 mmol) and tBuBrettPhos Pd G3 (120 mg, 0.14 mmol) in 1,4-dioxane (6 mL) with stirring under N₂ at RT. Heat the reaction to 65° C. After 3 hrs, cool the reaction to RT, pour into saturated aqueous ammonium chloride and extract with EtOAc (3×). Wash the combined organics with saturated aqueous NaCl, dry over anhydrous MgSO₄, filter and concentrate under vacuum. Triturate the material from Et₂O/hexanes and collect the resulting solid via filtration to give (R)—N—((S)-5,6-dichloro-8-hydroxy-2,3-dihydro-1H-pyrrolo[1,2-a]indol-1-yl)-2-methylpropane-2-sulfinamide (451 mg, 93%). ES-MS (m/z): (³⁵Cl/³⁷Cl) 360.8/362.8 (M+H).

### Intermediate 666

### (R)—N—((S)-5,6-Dichloro-8-ethoxy-2,3-dihydro-1H-pyrrolo[1,2-a]indol-1-yl)-2-methylpropane-2-sulfinamide

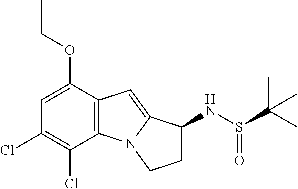 [SMILES: CCOc1cc(Cl)c(Cl)c2c1cc1n2CC[C@@H]1N[S@](=O)C(C)(C)C]

Add iodoethane (180 μL, 2.24 mmol) to (R)—N—((S)-5,6-dichloro-8-hydroxy-2,3-dihydro-1H-pyrrolo[1,2-a]indol-1-yl)-2-methylpropane-2-sulfinamide (450 mg, 1.24 mmol) and K₂CO₃ (225 mg, 1.63 mmol) in DMF (7.5 mL) with stirring, under N₂ and at RT. After 1 hr, pour the reaction into water and extract with EtOAc (3×). Wash the combined organics with water and saturated aqueous NaCl (2×), dry over anhydrous MgSO₄, filter and concentrate under vacuum. Purify the material by silica gel flash chromatography eluting with EtOAc and MeOH in CH₂Cl₂ to give (R)—N—((S)-5,6-dichloro-8-ethoxy-2,3-dihydro-1H-pyrrolo[1,2-a]indol-1-yl)-2-methylpropane-2-sulfinamide (373 mg, 77%). ES/MS (m/z): (³⁵Cl/³⁷Cl) 389.2/391.2 (M+H).

### Intermediate 667

### (R)—N—((S)-5,6-Dichloro-8-ethoxy-9-iodo-2,3-dihydro-1H-pyrrolo[1,2-a]indol-1-yl)-2-methylpropane-2-sulfinamide

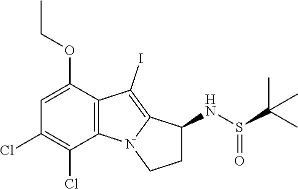 [SMILES: CCOc1cc(Cl)c(Cl)c2c1c(I)c1n2CC[C@@H]1N[S@](=O)C(C)(C)C]

Add N-iodosuccinimide (200 mg, 0.87 mmol) to (R)—N—((S)-5,6-dichloro-8-ethoxy-2,3-dihydro-1H-pyrrolo[1,2-a]indol-1-yl)-2-methylpropane-2-sulfinamide (310 mg, 0.80 mmol) in DMF (5 mL) with stirring, under N₂ and at RT. After 2 hrs., pour the reaction into saturated aqueous sodium thiosulfate and extract with EtOAc (3×). Wash the combined organics with saturated aqueous NaCl, dry over anhydrous MgSO₄, filter and concentrate under vacuum. Triturate the material from Et₂O/CH₂Cl₂/MeOH and collect the resulting solid via filtration to give (R)—N—((S)-5,6-dichloro-8-ethoxy-9-iodo-2,3-dihydro-1H-pyrrolo[1,2-a]indol-1-yl)-2-methylpropane-2-sulfinamide (441 mg, 99+% (nominal yield, used without further purification). ES/MS (m/z): (³⁵Cl/³⁷Cl) 514.8/516.8 (M+H).

### Intermediate 668

### (R)—N-((1 S)-5,6-Dichloro-8-ethoxy-9-(1-(tetrahydro-2H-pyran-2-yl)-1H-pyrazol-4-yl)-2,3-dihydro-1H-pyrrolo[1,2-a]indol-1-yl)-2-methylpropane-2-sulfinamide

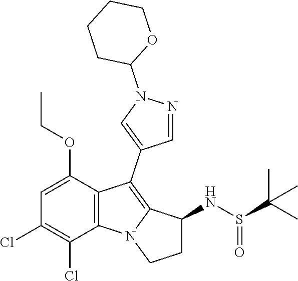 [SMILES: CCOc1cc(Cl)c(Cl)c2c1c(-c1cnn(C3CCCCO3)c1)c1n2CC[C@@H]1N[S@](=O)C(C)(C)C]

Add K₂CO₃ (215 mg, 1.55 mmol), CH₃CN (6 mL) and water (2 mL) to tri-tert-butylphosphonium tetrafluoroborate (25 mg, 0.08 mmol) and crotylpalladium chloride dimer (13 mg, 0.03 mmol) with stirring and at RT. After 30 min, add the mixture to a microwave vial containing (R)—N—((S)-5,6-dichloro-8-ethoxy-9-iodo-2,3-dihydro-1H-pyrrolo[1,2-a]indol-1-yl)-2-methylpropane-2-sulfinamide (400 mg, 0.78 mmol) and 1-(tetrahydro-2H-pyran-2-yl)-4-(4,4,5,5-tetramethyl-1,3-2-dioxaborolan-2-yl)-1H-pyrazole (440 mg, 1.55 mmol). Cap the vial and heat the reaction to 100° C. in a microwave reactor. After 1 hr, cool the reaction to RT, pour into H₂O and extract with EtOAc (3×). Wash the combined organics with saturated aqueous NaCl, dry over MgSO₄, filter and concentrate under vacuum. Purify the material via silica gel flash chromatography eluting with EtOAc and MeOH in CH₂Cl₂ to give (R)—N-((1S)-5,6-dichloro-8-ethoxy-9-(1-(tetrahydro-2H-pyran-2-yl)-1H-pyrazol-4-yl)-2,3-dihydro-1H-pyrrolo[1,2-a]indol-1-yl)-2-methylpropane-2-sulfinamide (225 mg, 0.54%). ES/MS (m/z): (³⁵Cl/³⁷Cl) 539.0/541.0 (M+H).

### Intermediate 669

### (S)-5,6-Dichloro-8-ethoxy-9-(1H-pyrazol-4-yl)-2,3-dihydro-1H-pyrrolo[1,2-a]indol-1-amine

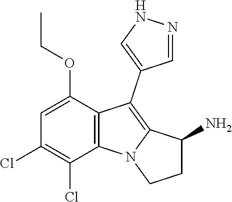 [SMILES: CCOc1cc(Cl)c(Cl)c2c1c(-c1cn[nH]c1)c1n2CC[C@@H]1N]

Add HCl (4.0 M) in 1,4-dioxane (525 μL, 2.1 mmol) to (R)—N-((1S)-5,6-dichloro-8-ethoxy-9-(1-(tetrahydro-2H-pyran-2-yl)-1H-pyrazol-4-yl)-2,3-dihydro-1H-pyrrolo[1,2-a]indol-1-yl)-2-methylpropane-2-sulfinamide (225 mg, 0.42 mmol) in 1,4-dioxane (3 mL) and MeOH (750 μL) with stirring, under N₂ and at RT. After 1 hr, concentrate the mixture under vacuum. Triturate the material from Et₂O and hexanes. Collect the resulting solid via filtration to give (S)-5,6-dichloro-8-ethoxy-9-(1H-pyrazol-4-yl)-2,3-dihydro-1H-pyrrolo[1,2-a]indol-1-amine as the hydrochloride salt (166 mg, 99+% (nominal yield, used without purification). ES/MS (m/z): (³⁵Cl/³⁷Cl) 350.8/352.8 (M+H).

### Intermediate 670

### (S)-2-((5,6-Dichloro-8-ethoxy-9-(1H-pyrazol-4-yl)-2,3-dihydro-1H-pyrrolo[1,2-a]indol-1-yl)amino)-2-oxoethylacetate

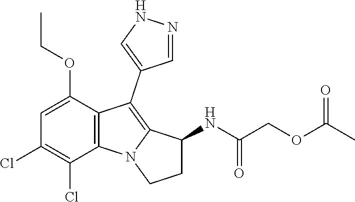 [SMILES: CCOc1cc(Cl)c(Cl)c2c1c(-c1cn[nH]c1)c1n2CC[C@@H]1NC(=O)COC(C)=O]

Add acetoxyacetyl chloride (50 μL, 0.45 mmol) to (S)-5,6-dichloro-8-ethoxy-9-(1H-pyrazol-4-yl)-2,3-dihydro-1H-pyrrolo[1,2-a]indol-1-amine (165 mg, 0.42 mmol, HCl salt) and Et₃N (180 μL, 1.3 mmol) in THF (3 mL) with stirring, under N₂ and at RT. After 1 hr, pour the reaction into saturated aqueous NaHCO₃ and extract with EtOAc (3×). Dry the combined organics over MgSO₄, filter and concentrate under vacuum. Triturate the material from Et₂O and collect the resulting solid via filtration to give (S)-2-((5,6-dichloro-8-ethoxy-9-(1H-pyrazol-4-yl)-2,3-dihydro-1H-pyrrolo[1,2-a]indol-1-yl)amino)-2-oxoethylacetate (172 mg, 87%). ES/MS (m/z): (³⁵Cl/³⁷Cl) 450.8/452.8 (M+H).

### EXAMPLE 343

### (S)—N-(5,6-Dichloro-8-ethoxy-9-(1H-pyrazol-4-yl)-2,3-dihydro-1H-pyrrolo[1,2-a]indol-1-yl)-2-hydroxyacetamide

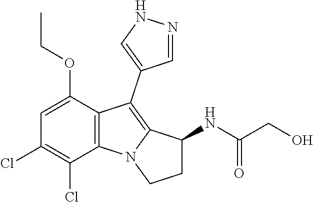 [SMILES: CCOc1cc(Cl)c(Cl)c2c1c(-c1cn[nH]c1)c1n2CC[C@@H]1NC(=O)CO]

Add lithium hydroxide (90 mg, 3.75 mmol) to (S)-2-((5,6-dichloro-8-ethoxy-9-(1H-pyrazol-4-yl)-2,3-dihydro-1H-pyrrolo[1,2-a]indol-1-yl)amino)-2-oxoethylacetate (170 mg, 0.375 mmol) in THF (3 mL), MeOH (2 mL) and water (1 mL) with stirring and at RT. Heat the reaction to 60° C. After 1 hour, cool the reaction to RT and pour it into saturated aqueous NaHCO₃. Extract with EtOAc (3×). Dry the combined organics over MgSO₄, filter and concentrate under vacuum. Purify the material via reverse phase chromatography to give (S)—N-(5,6-dichloro-8-ethoxy-9-(1H-pyrazol-4-yl)-2,3-dihydro-1H-pyrrolo[1,2-a]indol-1-yl)-2-hydroxyacetamide (68 mg, 0.167 mmol, 43%). ES-MS (m/z): (³⁵Cl/³⁷Cl) 409.2/411.2 (M+H).

### Intermediate 671

### tert-Butyl (S)-(8-bromo-5,6-dichloro-2,3-dihydro-1H-pyrrolo[1,2-a]indol-1-yl)carbamate

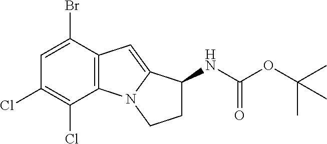 [SMILES: CC(C)(C)OC(=O)N[C@H]1CCn2c1cc1c(Br)cc(Cl)c(Cl)c12]

Add di-tert-butyl dicarbonate (650 mg, 2.98 mmol) to (S)-8-bromo-5,6-dichloro-2,3-dihydro-1H-pyrrolo[1,2-a]indol-1-amine hydrochloride (840 mg, 2.35 mmol) and Et₃N (1 mL, 7.17 mmol) in DMF (10 mL) with stirring, under N₂ and at RT. Heat the reaction to 50° C. After 2 hrs, cool the reaction to RT, pour into H₂O and extract with EtOAc (3×). Dry the combined organics over MgSO₄, filter and concentrate under vacuum to give tert-butyl (S)-(8-bromo-5,6-dichloro-2,3-dihydro-1H-pyrrolo[1,2-a]indol-1-yl)carbamate (757 mg, 76%). ES/MS (m/z): (³⁵Cl/³⁷Cl) 364.6/366.6 (M+1 with loss of tert-butyl).

### Intermediate 672

### tert-Butyl (S)-(5,6-dichloro-8-hydroxy-2,3-dihydro-1H-pyrrolo[1,2-a]indol-1-yl)carbamate

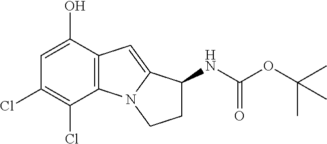 [SMILES: CC(C)(C)OC(=O)N[C@H]1CCn2c1cc1c(O)cc(Cl)c(Cl)c12]

Add sodium tert-butoxide (430 mg, 4.5 mmol) and H₂O (3 mL) to tert-butyl (S)-(8-bromo-5,6-dichloro-2,3-dihydro-1H-pyrrolo[1,2-a]indol-1-yl)carbamate (750 mg, 1.8 mmol) and tBuBrettPhos Pd G3 (160 mg, 0.18 mmol) in 1,4-dioxane (9 mL) with stirring, under N₂ and at RT. Heat the reaction to 65° C. After 3 hrs., cool the reaction to RT, pour into saturated aqueous ammonium chloride and extract with EtOAc (3×). Wash the combined organics with saturated aqueous NaCl, dry over MgSO₄, filter and concentrate under vacuum. Triturate the material from Et₂O/hexanes and collect the resulting solid via filtration to give tert-butyl (S)-(5,6-dichloro-8-hydroxy-2,3-dihydro-1H-pyrrolo[1,2-a]indol-1-yl)carbamate (543 mg, 85%). ES/MS (m/z): (³⁵Cl/³⁷Cl) 356.8/358.9 (M+H).

### Intermediate 673

### tert-Butyl (S)-(5,6-dichloro-8-(cyanomethoxy)-2,3-dihydro-1H-pyrrolo[1,2-a]indol-1-yl)carbamate

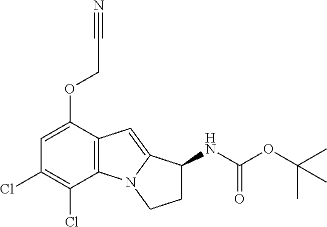 [SMILES: CC(C)(C)OC(=O)N[C@H]1CCn2c1cc1c(OCC#N)cc(Cl)c(Cl)c12]

Add bromoacetonitrile (275 mg, 2.3 mmol) to tert-butyl (S)-(5,6-dichloro-8-hydroxy-2,3-dihydro-1H-pyrrolo[1,2-a]indol-1-yl)carbamate (540 mg, 1.5 mmol) and K₂CO₃ (₂25 mg, 1.6 mmol) in DMF (8 mL) with stirring, under N₂ and at RT. After 2 hrs., pour the reaction into H₂O and extract with EtOAc (3×). Wash the combined organics with H₂O and saturated aqueous NaCl, dry over MgSO₄, filter and concentrate under vacuum. Triturate the material from Et₂O/hexanes and collect the resulting solid via filtration to give tert-butyl (S)-(5,6-dichloro-8-(cyanomethoxy)-2,3-dihydro-1H-pyrrolo[1,2-a]indol-1-yl)carbamate (524 mg, 87%). ES/MS (m/z): (³⁵Cl/³⁷Cl) 339.8/341.8 (M+H with loss of tert-butyl).

### Intermediate 674

### tert-Butyl (S)-(5,6-dichloro-8-(cyanomethoxy)-9-iodo-2,3-dihydro-1H-pyrrolo[1,2-a]indol-1-yl)carbamate

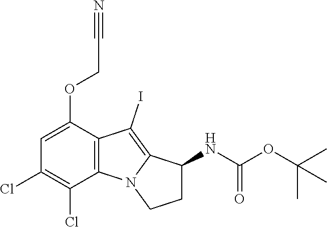 [SMILES: CC(C)(C)OC(=O)N[C@H]1CCn2c1c(I)c1c(OCC#N)cc(Cl)c(Cl)c12]

Add N-iodosuccinimide (333 mg, 1.48 mmol) to tert-butyl (S)-(5,6-dichloro-8-(cyanomethoxy)-2,3-dihydro-1H-pyrrolo[1,2-a]indol-1-yl)carbamate (520 mg, 1.41 mmol) in DMF (7 mL) with stirring, under N₂ and at RT. After 2 hrs., pour the reaction into saturated aqueous sodium thiosulfate and extract with EtOAc (3×). Wash the combined organics with saturated aqueous NaCl, dry over MgSO₄, filter and concentrate under vacuum. Triturate the material from Et₂O/CH₂Cl₂ and collect the resulting solid via filtration to give tert-butyl (S)-(5,6-dichloro-8-(cyanomethoxy)-9-iodo-2,3-dihydro-1H-pyrrolo[1,2-a]indol-1-yl)carbamate (627 mg, 92%). ES/MS (m/z): (³⁵Cl/³⁷Cl) 465.6/467.6 (M+H with loss of tert-butyl).

### Intermediate 675

### tert-Butyl ((1S)-5,6-dichloro-8-(cyanomethoxy)-9-(1-(tetrahydro-2H-pyran-2-yl)-1H-pyrazol-4-yl)-2,3-dihydro-1H-pyrrolo[1,2-a]indol-1-yl)carbamate

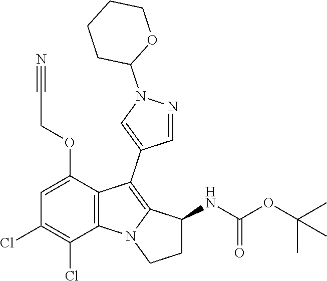 [SMILES: CC(C)(C)OC(=O)N[C@H]1CCn2c1c(-c1cnn(C3CCCCO3)c1)c1c(OCC#N)cc(Cl)c(Cl)c12]

Add K₂CO₃ (₂15 mg, 1.55 mmol), CH₃CN (12 mL) and H₂O (3 mL) to tri-tert-butylphosphonium tetrafluoroborate (36 mg, 0.12 mmol) and crotylpalladium chloride dimer (19 mg, 0.048 mmol) with stirring and at RT. After 30 minutes, add the mixture to a microwave vial containing tert-butyl (S)-(5,6-dichloro-8-(cyanomethoxy)-9-iodo-2,3-dihydro-1H-pyrrolo[1,2-a]indol-1-yl)carbamate (625 mg, 1.2 mmol) and 1-(tetrahydro-2H-pyran-2-yl)-4-(4,4,5,5-tetramethyl-1,3-2-dioxaborolan-2-yl)-1H-pyrazole (680 mg, 2.45 mmol). Cap the vial and heat the reaction to 100° C. in a microwave oven. After 1 hr., cool the reaction to RT and pour it into water. Extract the material with EtOAc (3×). Wash the combined organics with saturated aqueous NaCl, dry over MgSO₄, filter and concentrate under vacuum. Purify the material via silica gel flash chromatography eluting with EtOAc and MeOH in DCM to give tert-butyl ((1S)-5,6-dichloro-8-(cyanomethoxy)-9-(1-(tetrahydro-2H-pyran-2-yl)-1H-pyrazol-4-yl)-2,3-dihydro-1H-pyrrolo[1,2-a]indol-1-yl)carbamate (371 mg, 57%). ES/MS (m/z): (³⁵Cl/³⁷Cl) 545.8/548.0 (M+H).

### Intermediate 676

### (S)-2-((-Amino-5,6-dichloro-9-(1H-pyrazol-4-yl)-2,3-dihydro-1H-pyrrolo[1,2-a]indol-8-yl)oxy)acetonitrile

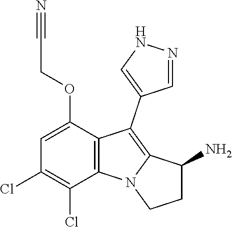 [SMILES: N#CCOc1cc(Cl)c(Cl)c2c1c(-c1cn[nH]c1)c1n2CC[C@@H]1N]

Add TFA (1 mL) to tert-butyl ((1S)-5,6-dichloro-8-(cyanomethoxy)-9-(1-(tetrahydro-2H-pyran-2-yl)-1H-pyrazol-4-yl)-2,3-dihydro-1H-pyrrolo[1,2-a]indol-1-yl)carbamate (370 mg, 0.67 mmol) in DCM (3 mL) with stirring, under N₂ and at RT. After 2 hrs., concentrate the reaction under vacuum. Take up the residue in EtOAc and pour into saturated aqueous NaHCO₃. Separate the layers and extract the aqueous with EtOAc. Dry the combined organics over MgSO₄, filter and concentrate under vacuum to give (S)-2-((1-amino-5,6-dichloro-9-(1H-pyrazol-4-yl)-2,3-dihydro-1H-pyrrolo[1,2-a]indol-8-yl)oxy)acetonitrile (252 mg, 99+% (nominal yield), used without further purification). ES/MS (m/z): (³⁵Cl/³⁷Cl) 362.0/364.0 (M+H).

### EXAMPLE 344

### (S)—N-(5,6-Dichloro-8-(cyanomethoxy)-9-(1H-pyrazol-4-yl)-2,3-dihydro-1H-pyrrolo[1,2-a]indol-1-yl)-2-hydroxyacetamide

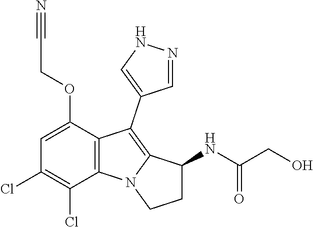 [SMILES: N#CCOc1cc(Cl)c(Cl)c2c1c(-c1cn[nH]c1)c1n2CC[C@@H]1NC(=O)CO]

Add HATU (320 mg, 0.84 mmol) to (S)-2-((1-amino-5,6-dichloro-9-(1H-pyrazol-4-yl)-2,3-dihydro-1H-pyrrolo[1,2-a]indol-8-yl)oxy)acetonitrile (250 mg, 0.69 mmol), glycolic acid (65 mg, 0.85 mmol) and Et₃N (500 μL, 3.58 mmol) in DMF (7 mL) with stirring and at RT. After 18 hrs., pour the reaction into H₂O and extract with EtOAc (3×). Wash the combined organics with saturated aqueous NaCl, dry over MgSO₄, filter and concentrate under vacuum. Purify the material via reverse phase chromatography and trituration from MeOH/Et₂₀ to give (S)—N-(5,6-dichloro-8-(cyanomethoxy)-9-(1H-pyrazol-4-yl)-2,3-dihydro-1H-pyrrolo[1,2-a]indol-1-yl)-2-hydroxyacetamide (97 mg, 32%). ES/MS (m/z): (³⁵Cl/³⁷Cl) 419.8/421.8 (M+H).

### EXAMPLES 345 & 346

### N-(5,6-Dichloro-9-(1H-pyrazol-4-yl)-2,3-dihydro-1H-pyrrolo[1,2-a]indol-1-yl)-2-hydroxyacetamide

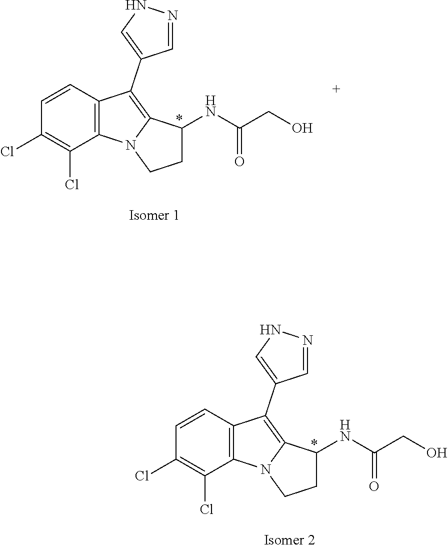 [SMILES: O=C(CO)NC1CCn2c1c(-c1cn[nH]c1)c1ccc(Cl)c(Cl)c12 ; O=C(CO)NC1CCn2c1c(-c1cn[nH]c1)c1ccc(Cl)c(Cl)c12]

Purify N-(5,6-dichloro-9-(1H-pyrazol-4-yl)-2,3-dihydro-1H-pyrrolo[1,2-a]indol-1-yl)-2-hydroxyacetamide (31 mg, 0.085 mmol) by SFC to give N-(5,6-dichloro-9-(1H-pyrazol-4-yl)-2,3-dihydro-1H-pyrrolo[1,2-a]indol-1-yl)-2-hydroxyacetamide as single stereoisomers (Isomer 1-12 mg, 0.033 mmol, 39%; Isomer 2-15 mg, 0.041 mmol, 48%). Rt=1.15 (99% ee) and 2.55 (99% ee) minutes. Column: Chiralpak AD-H, 4.6×100 mm; Mobile Phase: 30% MeOH: 70% CO₂; Flow Rate: 5 mL/min. ES/MS (m/z): (³⁵Cl/³⁷Cl) 365.0/367.2 (M+H).

### Intermediate 677

### 4-Bromo-6,7-dichloroindoline

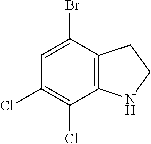 [SMILES: Clc1cc(Br)c2c(c1Cl)NCC2]

Add NaBH₄ 2.1 g, 55.5 mmol) carefully to 4-bromo-6,7-dichloro-1H-indole (5.0 g, 18.9 mmol) in TFA (47 mL) with stirring, under N₂ and at RT. After 3 hrs, dilute the reaction with CH₂Cl₂ (100 mL) and carefully add to a stirred solution of 2.0 M aqueous NaOH dropwise. Separate the layers and extract with CH₂Cl₂ (2×). Dry the combined organics over MgSO₄, filter and concentrate under vacuum. Purify via silica gel flash chromatography eluting with EtOAc in hexanes to give 4-bromo-6,7-dichloroindoline (4.45 g, 89%). ¹H NMR (DMSO-d6): 6.91 (s, 1H), 6.43-6.40 (m, 1H), 3.61 (td, J=8.9, 1.5 Hz, 2H), 3.02 (t, J=8.9 Hz, 2H).

### Intermediate 678

### Diethyl 2-(2-(4-bromo-6,7-dichloroindolin-1-yl)ethyl)malonate

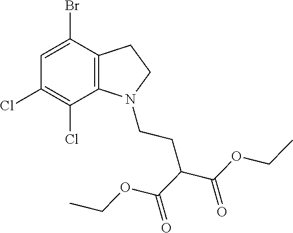 [SMILES: CCOC(=O)C(CCN1CCc2c(Br)cc(Cl)c(Cl)c21)C(=O)OCC]

Add ytterbium triflate (517 mg, 0.83 mmol) to 4-bromo-6,7-dichloroindoline (4.47 g, 16.7 mmol) and diethyl-1,1-cyclopropanedicarboxylate (3.1 g, 16.7 mmol) in toluene (55 mL) with stirring, under N₂ and at RT. Heat the reaction to reflux. After 3 hrs, cool the reaction to RT and concentrate under vacuum. Purify the material via silica gel flash chromatography eluting with EtOAc in hexanes to give diethyl 2-(2-(4-bromo-6,7-dichloroindolin-1-yl)ethyl)malonate (7.2 g, 95%). ES/MS (m/z): (³⁵Cl/³⁷Cl) 454.0/456.0 (M+H).

### Intermediate 679

### Diethyl 8-bromo-5,6-dichloro-2,3-dihydro-1H-pyrrolo[1,2-a]indole-1,1-dicarboxylate

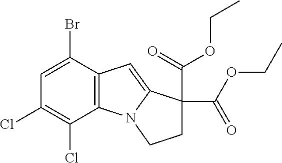 [SMILES: CCOC(=O)C1(C(=O)OCC)CCn2c1cc1c(Br)cc(Cl)c(Cl)c12]

Add triacetoxymanganese dihydrate (21 g, 79 mmol) to diethyl 2-(2-(4-bromo-6,7-dichloroindolin-1-yl)ethyl)malonate (7.2 g, 16.0 mmol) in MeOH (80 mL) with stirring, under N₂ and at RT. Heat the reaction to reflux. After 3 hrs., cool the reaction to RT and concentrate under vacuum. Dilute with EtOAc, pour into H₂O and extract with EtOAc (3×). Wash the combined organics with saturated aqueous NaCl, dry over MgSO₄, filter, and concentrate under vacuum. Purify the material via silica gel flash chromatography eluting with EtOAc in hexanes to give diethyl 8-bromo-5,6-dichloro-2,3-dihydro-1H-pyrrolo[1,2-a]indole-1,1-dicarboxylate (3.2 g, 45%). ES/MS (m/z): (³⁵Cl/³⁷Cl) 450.2/452.2 (M+H).

### Intermediate 680

### Ethyl 8-bromo-5,6-dichloro-2,3-dihydro-1H-pyrrolo[1,2-a]indole-1-carboxylate

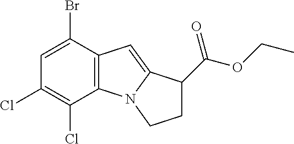 [SMILES: CCOC(=O)C1CCn2c1cc1c(Br)cc(Cl)c(Cl)c12]

Add lithium chloride (1.4 g, 33 mmol) to diethyl 8-bromo-5,6-dichloro-2,3-dihydro-1H-pyrrolo[1,2-a]indole-1,1-dicarboxylate (3.0 g, 6.7 mmol) in DMF (22 mL) and H₂O (220 μL) with stirring and at RT. Heat the reaction to 150° C. After 2 hrs., cool the reaction to RT, pour into water and extract with EtOAc (3×). Wash the combined organics with saturated aqueous NaCl (2×), dry over MgSO₄, filter, and concentrate under vacuum. Purify the material via silica gel flash chromatography eluting with EtOAc in hexanes to give ethyl 8-bromo-5,6-dichloro-2,3-dihydro-1H-pyrrolo[1,2-a]indole-1-carboxylate (1.47 g, 58%). ES/MS (m/z): (³⁵Cl/³⁷Cl) 377.6/379.8 (M+H).

### Intermediate 681

### (8-Bromo-5,6-dichloro-2,3-dihydro-1H-pyrrolo[1,2-a]indol-1-yl)methanol

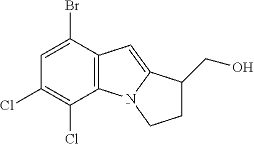 [SMILES: OCC1CCn2c1cc1c(Br)cc(Cl)c(Cl)c12]

Add DIBAL-H [1.0 M in toluene, 7.7 mL, 7.7 mmol) to ethyl 8-bromo-5,6-dichloro-2,3-dihydro-1H-pyrrolo[1,2-a]indole-1-carboxylate (725 mg, 1.9 mmol) in THF (3.85 mL) and CH₂Cl₂ (3.85 mL) with stirring, under N₂ and at −78° C. Allow to warm to RT with stirring for 16-18 hrs. Quench by careful addition of a saturated aqueous solution of potassium sodium tartrate tetrahydrate. After 10 min, pour into water and extract with EtOAc (3×). Wash the combined organics with saturated aqueous NaCl, dry over MgSO₄, filter, and concentrate under vacuum. Purify by silica gel flash chromatography eluting with EtOAc in hexanes to give (8-bromo-5,6-dichloro-2,3-dihydro-1H-pyrrolo[1,2-a]indol-1-yl)methanol (650 mg, 99+% (nominal yield, used without purification). ES/MS (m/z): (³⁵Cl/³⁷Cl) 335.8/337.8 (M+H).

### Intermediate 682

### 5,6-Dichloro-1-(hydroxy methyl)-2,3-dihydro-1H-pyrrolo[1,2-a]indol-8-ol

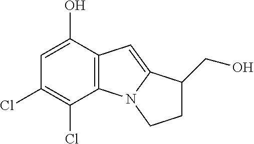 [SMILES: OCC1CCn2c1cc1c(O)cc(Cl)c(Cl)c12]

Add sodium tert-butoxide (463 mg, 4.8 mmol) and water (3.85 mL) to (8-bromo-5,6-dichloro-2,3-dihydro-1H-pyrrolo[1,2-a]indol-1-yl)methanol (645 mg, 1.9 mmol) and tBuBrettPhos Pd G3 (165 mg, 0.19 mmol) in 1,4-dioxane (9.5 mL) with stirring under N₂ and at RT. Heat the reaction to 65° C. After 3 hrs., cool the reaction to RT, pour into saturated aqueous ammonium chloride and extract with EtOAc (3×). Wash the combined organics with saturated aqueous NaCl, dry over MgSO₄, filter and concentrate under vacuum. Purify by silica gel flash chromatography eluting with EtOAc and MeOH in CH₂Cl₂ to give 5,6-dichloro-1-(hydroxymethyl)-2,3-dihydro-1H-pyrrolo (171 mg, 33%). ES/MS (m/z): (³⁵Cl/³⁷Cl) 271.8/273.8 (M+H).

### Intermediate 683

### 2-((5,6-Dichloro-1-(hydroxymethyl)-2,3-dihydro-1H-pyrrolo[1,2-a]indol-8-yl)oxy)acetonitrile

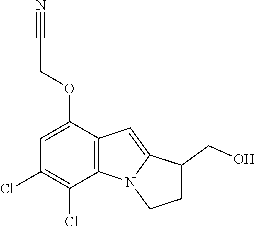 [SMILES: N#CCOc1cc(Cl)c(Cl)c2c1cc1n2CCC1CO]

Add bromoacetonitrile (65 μL, 0.95 mmol) to 5,6-dichloro-1-(hydroxymethyl)-2,3-dihydro-1H-pyrrolo[1,2-a]indol-8-ol (170 mg, 0.63 mmol) and K₂CO₃ (₂17 mg, 1.57 mmol) in DMF (4 mL) with stirring, under N₂ and at RT. After 2 hrs., pour the reaction into water and extract with EtOAc (3×). Wash the combined organics with water and saturated aqueous NaCl, dry over MgSO₄, filter, and concentrate under vacuum. Purify by silica gel flash chromatography eluting with EtOAc in hexanes to give 2-((5,6-dichloro-1-(hydroxymethyl)-2,3-dihydro-1H-pyrrolo[1,2-a]indol-8-yl)oxy)acetonitrile (135 mg, 70%). ES/MS (m/z): (³⁵Cl/³⁷Cl) 311.0/313.0 (M+H).

### Intermediate 684

### 2-((5,6-Dichloro-1-(hydroxymethyl)-9-iodo-2,3-dihydro-1H-pyrrolo[1,2-a]indol-8-yl)oxy)acetonitrile

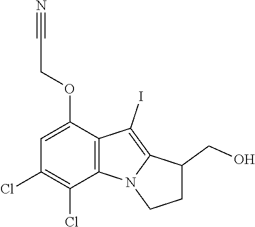 [SMILES: N#CCOc1cc(Cl)c(Cl)c2c1c(I)c1n2CCC1CO]

Add N-iodosuccinimide (106 mg, 0.47 mmol) to 2-((5,6-dichloro-1-(hydroxymethyl)-2,3-dihydro-1H-pyrrolo[1,2-a]indol-8-yl)oxy)acetonitrile (135 mg, 0.43 mmol) in DMF (3 mL) with stirring under N₂ and at RT. After 2 hrs., pour the reaction into saturated aqueous sodium thiosulfate and extract with EtOAc (3×). Wash the combined organics with saturated aqueous NaCl, dry over MgSO₄, filter, and concentrate under vacuum. Triturate the material from Et₂O/hexanes and collect the resulting solid via filtration to give 2-((5,6-dichloro-1-(hydroxymethyl)-9-iodo-2,3-dihydro-1H-pyrrolo[1,2-a]indol-8-yl)oxy)acetonitrile (160 mg, 85%). ES/MS (m/z): (³⁵Cl/³⁷Cl) 436.6/438.6 (M+H).

### Intermediate 685

### 2-((5, 6-Dichloro-1-(hydroxymethyl)-9-(1-(tetrahydro-2H-pyran-2-yl)-1H-pyrazol-4-yl)-2,3-dihydro-1H-pyrrolo[1,2-a]indol-8-yl)oxy)acetonitrile

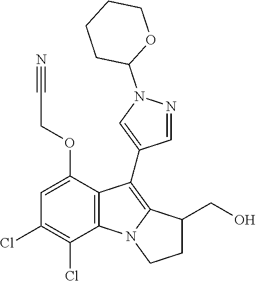 [SMILES: N#CCOc1cc(Cl)c(Cl)c2c1c(-c1cnn(C3CCCCO3)c1)c1n2CCC1CO]

Add K₂CO₃ (104 mg, 0.75 mmol), CH₃CN (5 mL) and H₂O (2 mL) to tri-tert-butylphosphonium tetrafluoroborate (12 mg, 0.04 mmol) and crotylpalladium chloride dimer (6 mg, 0.02 mmol) with stirring and at RT. After 30 min, add the mixture to a microwave vial containing 2-((5,6-dichloro-1-(hydroxymethyl)-9-iodo-2,3-dihydro-1H-pyrrolo[1,2-a]indol-8-yl)oxy)acetonitrile (160 mg, 0.37 mmol) and 1-(tetrahydro-2H-pyran-2-yl)-4-(4,4,5,5-tetramethyl-1,3-2-dioxaborolan-2-yl)-1H-pyrazole (210 mg, 0.75 mmol). Cap the vial and heat the reaction to 100° C. in a microwave oven. After 1 hr., cool the reaction to RT and pour it into water. Extract the material with EtOAc (3×). Wash the combined organics with saturated aqueous NaCl, dry over MgSO₄, filter, and concentrate under vacuum. Purify by silica gel flash chromatography eluting with EtOAc and MeOH in CH₂Cl₂ to give 2-((5,6-dichloro-1-(hydroxymethyl)-9-(1-(tetrahydro-2H-pyran-2-yl)-1H-pyrazol-4-yl)-2,3-dihydro-1H-pyrrolo[1,2-a]indol-8-yl)oxy)acetonitrile (90 mg, 53%). ES/MS (m/z): (³⁵Cl/³⁷Cl) 460.8/463.0 (M+H).

### EXAMPLE 347

### 2-((5,6-Dichloro-1-(hydroxymethyl)-9-(1H-pyrazol-4-yl)-2,3-dihydro-1H-pyrrolo[1,2-a]indol-8-yl)oxy)acetonitrile

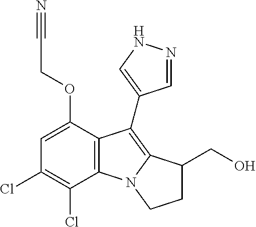 [SMILES: N#CCOc1cc(Cl)c(Cl)c2c1c(-c1cn[nH]c1)c1n2CCC1CO]

Add TFA (160 μL, 2.0 mmol) to 2-((5,6-dichloro-1-(hydroxymethyl)-9-(1-(tetrahydro-2H-pyran-2-yl)-1H-pyrazol-4-yl)-2,3-dihydro-1H-pyrrolo[1,2-a]indol-8-yl)oxy)acetonitrile (90 mg, 0.19 mmol) in CH₂Cl₂ (1 mL) with stirring, under N₂ and at RT. After 1 hr., concentrate the reaction under vacuum. Take up the material in EtOAc and pour into saturated aqueous NaHCO₃. Separate the layers and extract with EtOAc (2×). Dry the combined organics over MgSO₄, filter, and concentrate under vacuum. Purify the material via silica gel reverse phase chromatography to give 2-((5,6-dichloro-1-(hydroxymethyl)-9-(1H-pyrazol-4-yl)-2,3-dihydro-1H-pyrrolo[1,2-a]indol-8-yl)oxy)acetonitrile (34 mg, 47%). ES/MS (m/z): (³⁵Cl/³⁷Cl) 376.8/378.0 (M+H).

### Intermediate 686

### 1-Bromo-3,4-dichloro-6,7,8,9-tetrahydropyrido[1,2-a]indol-7-ol

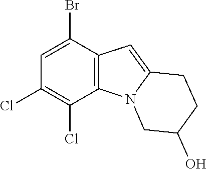 [SMILES: OC1CCc2cc3c(Br)cc(Cl)c(Cl)c3n2C1]

Add NaBH₄ (120 mg, 3.01 mmol) to 1-bromo-3,4-dichloro-8,9-dihydropyrido[1,2-a]indol-7(6H)-one (300 mg, 0.811 mmol) in MeOH (6 mL). Stir at RT under N₂ for 2 hours. After complete consumption of starting material, quench by addition of saturated ammonium chloride solution (10 mL) at 0° C. and dilute with H₂O. Collect the precipitate by filtration and dry under vacuum to give 1-bromo-3,4-dichloro-6,7,8,9-tetrahydropyrido[1,2-a]indol-7-ol (290 mg, 99+%). ES/MS (m/z): (³⁵Cl/³⁷Cl) 335.8/337.8 (M+H).

### Intermediate 687

### 3,4-Dichloro-6,7,8,9-tetrahydropyrido[1,2-a]indole-1,7-diol

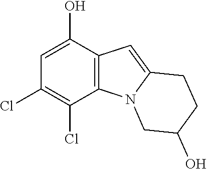 [SMILES: Oc1cc(Cl)c(Cl)c2c1cc1n2CC(O)CC1]

Suspend 1-bromo-3,4-dichloro-6,7,8,9-tetrahydropyrido[1,2-a]indol-7-ol (290 mg, 0.779 mmol), sodium 2-methylpropan-2-olate (189 mg, 212 μL, 1.95 mmol) and tBuBrettPhos Pd G3 (88.3 mg, 0.101 mmol) in 1,4-dioxane (4 mL) and H₂O (0.8 mL). Stir at 65° C. under N₂. After complete consumption of starting material, concentrate under vacuum, dilute with H₂O, and extract with EtOAc (3×). Dry over anhydrous Na₂SO₄, filter and concentrate under vacuum. Purify by silica gel flash chromatography eluting with EtOAc in petroleum ether to give 3,4-dichloro-6,7,8,9-tetrahydropyrido[1,2-a]indole-1,7-diol (150 mg, 50% yield). ES/MS (m/z): (³⁵Cl/³⁷Cl) 271.8/273.8 (M+H).

### Intermediate 688

### 1-(3-((tert-butyldimethylsilyl)oxy)propoxy)-3,4-dichloro-6,7,8,9-tetrahydropyrido[1,2-a]indol-7-ol

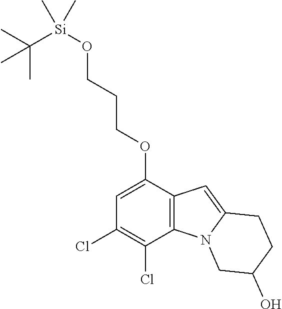 [SMILES: CC(C)(C)[Si](C)(C)OCCCOc1cc(Cl)c(Cl)c2c1cc1n2CC(O)CC1]

Suspend 3,4-dichloro-6,7,8,9-tetrahydropyrido[1,2-a]indole-1,7-diol (150 mg, 0.386 mmol) and potassium carbonate (107 mg, 0.772 mmol) in DMF (5 mL) and add tert-butyl(3-iodopropoxy)dimethylsilane (151 mg, 0.502 mmol). Stir at room temperature under N₂. After complete consumption of starting material, dilute with H₂O and extract with EtOAc (3×). Wash the combined organics with saturated aqueous NaCl (2×), dry over anhydrous Na₂SO₄, filter, and concentrate under vacuum. Purify by flash silica gel chromatography eluting with EtOAc in petroleum ether to give 1-(3-((tert-butyldimethylsilyl)oxy)propoxy)-3,4-dichloro-6,7,8,9-tetrahydropyrido[1,2-a]indol-7-ol (110 mg, 58% yield). ¹H NMR (DMSO-d6): 6.72 (s, 1H), 6.22 (s, 1H), 5.23 (t, 1H), 4.62 (dd, 1H), 4.40 (dd, 1H), 4.18 (m, 1H), 4.13 (t, 2H), 3.78 (t, 2H), 3.02 (m, 1H), 2.84 (m, 1H), 1.91 (m, 3H), 1.80 (m, 1H), 0.85 (s, 9H), 0.17 (s, 6H).

### Intermediate 689

### 3,4-Dichloro-1-(3-hydroxypropoxy)-10-iodo-6,7,8,9-tetrahydropyrido[1,2-a]indol-7-ol

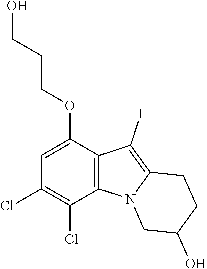 [SMILES: OCCCOc1cc(Cl)c(Cl)c2c1c(I)c1n2CC(O)CC1]

Dissolve 1-(3-((tert-butyldimethylsilyl)oxy)propoxy)-3,4-dichloro-6,7,8,9-tetrahydropyrido[1,2-a]indol-7-ol (110 mg, 0.210 mmol) in DMF (3 mL) and add NIS (54.8 mg, 0.231 mmol). Stir at RT under N₂. Upon complete consumption of the starting material, dilute with water and extract with EtOAc (2×). Wash the combined organic layers sequentially with saturated aqueous sodium sulfite and saturated aqueous NaCl (2×), dry over anhydrous sodium sulfate, filter, and concentrate under vacuum. Purify by flash silica gel eluting with EtOAc in petroleum ether to give 3,4-dichloro-1-(3-hydroxypropoxy)-10-iodo-6,7,8,9-tetrahydropyrido[1,2-a]indol-7-ol (60 mg, 54%). ES/MS (m/z): (³⁵Cl/³⁷Cl) 455.8/457.7 (M+H).

### Intermediate 690

### 3,4-Dichloro-1-(3-hydroxypropoxy)-10-(1-(tetrahydro-2H-pyran-2-yl)-1H-pyrazol-4-yl)-6,7,8,9-tetrahydropyrido[1,2-a]indol-7-ol

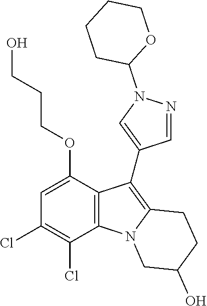 [SMILES: OCCCOc1cc(Cl)c(Cl)c2c1c(-c1cnn(C3CCCCO3)c1)c1n2CC(O)CC1]

Suspend 3,4-dichloro-1-(3-hydroxypropoxy)-10-iodo-6,7,8,9-tetrahydropyrido[1,2-a]indol-7-ol (146 mg, 0.278 mmol), 1-(tetrahydro-2H-pyran-2-yl)-4-(4,4,5,5-tetramethyl-1,3,2-dioxaborolan-2-yl)-1H-pyrazole (116 mg, 0.418 mmol), 1,1′-Bis(di-t-butylphosphino)ferrocene palladium dichloride (18.2 mg, 0.0278 mmol) and sodium carbonate (73.8 mg, 0.696 mmol) in 1,4-dioxane (4 mL) and H₂O (1 mL) and stir under N₂ at 90° C. Upon complete consumption of the starting material, concentrate under vacuum, dilute with H₂O, and extract with EtOAc (3×). Dry the combined organics over anhydrous sodium sulfate, filter, and concentrate under vacuum. Purify by flash silica gel chromatography eluting with methanol in DCM to give 3,4-dichloro-1-(3-hydroxypropoxy)-10-(1-(tetrahydro-2H-pyran-2-yl)-1H-pyrazol-4-yl)-6,7,8,9-tetrahydropyrido[1,2-a]indol-7-ol (96 mg, 72%). ES/MS (m/z): (³⁵Cl/³⁷Cl) 480.2/482.2 (M+H).

### EXAMPLE 348

### 3,4-Dichloro-1-(3-hydroxypropoxy)-10-(1H-pyrazol-4-yl)-6,7,8,9-tetrahydropyrido[1,2-a]indol-7-ol

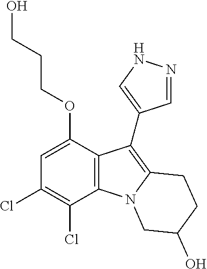 [SMILES: OCCCOc1cc(Cl)c(Cl)c2c1c(-c1cn[nH]c1)c1n2CC(O)CC1]

Dissolve 3,4-dichloro-1-(3-hydroxypropoxy)-10-(1-(tetrahydro-2H-pyran-2-yl)-1H-pyrazol-4-yl)-6,7,8,9-tetrahydropyrido[1,2-a]indol-7-ol (96 mg, 0.20 mmol) in THF (1.5 mL) and add HCl (6 M in water, 1.5 mL, 9.0 mmol). Stir at RT under N₂. After complete consumption of the starting material, dilute with DMF (3 mL) and purify by prep-HPLC to give 3,4-dichloro-1-(3-hydroxypropoxy)-10-(1H-pyrazol-4-yl)-6,7,8,9-tetrahydropyrido[1,2-a]indol-7-ol (47.9 mg, 59%). ES/MS (m/z): (³⁵Cl/³⁷Cl) 396.0/398.0 (M+H).

Compounds 95-348 are listed in Table 6.

TABLE 6

| Ex. No. | Compound |
|---|---|
| 95 | 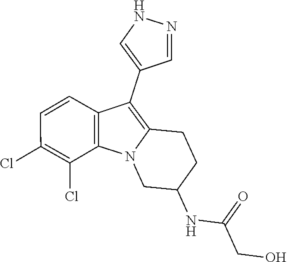 [SMILES: O=C(CO)NC1CCc2c(-c3cn[nH]c3)c3ccc(Cl)c(Cl)c3n2C1] |
| 96 | 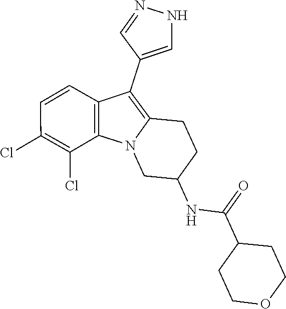 [SMILES: O=C(NC1CCc2c(-c3cn[nH]c3)c3ccc(Cl)c(Cl)c3n2C1)C1CCOCC1] |
| 99 | 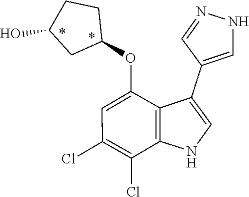 [SMILES: O[C@@H]1CC[C@@H](Oc2cc(Cl)c(Cl)c3[nH]cc(-c4cn[nH]c4)c23)C1] |
| 100 | 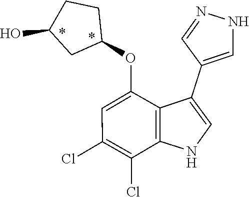 [SMILES: O[C@H]1CC[C@@H](Oc2cc(Cl)c(Cl)c3[nH]cc(-c4cn[nH]c4)c23)C1] |
| 101 | 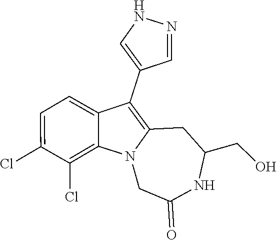 [SMILES: O=C1Cn2c(c(-c3cn[nH]c3)c3ccc(Cl)c(Cl)c32)CC(CO)N1] |
| 102 | 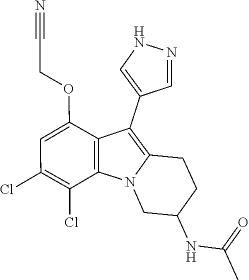 [SMILES: CC(=O)NC1CCc2c(-c3cn[nH]c3)c3c(OCC#N)cc(Cl)c(Cl)c3n2C1] |
| 103 | 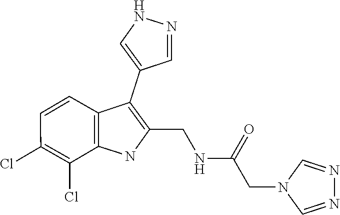 [SMILES: O=C(Cn1cnnc1)NCc1[nH]c2c(Cl)c(Cl)ccc2c1-c1cn[nH]c1] |
| 104 | 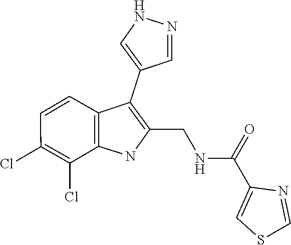 [SMILES: O=C(NCc1[nH]c2c(Cl)c(Cl)ccc2c1-c1cn[nH]c1)c1cscn1] |
| 105 | 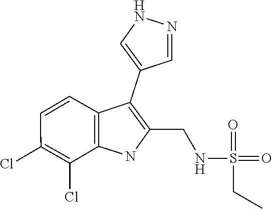 [SMILES: CCS(=O)(=O)NCc1[nH]c2c(Cl)c(Cl)ccc2c1-c1cn[nH]c1] |
| 106 | 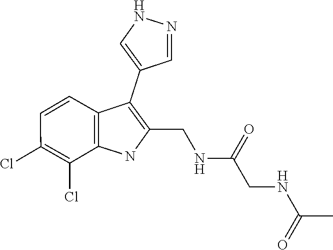 [SMILES: CC(=O)NCC(=O)NCc1[nH]c2c(Cl)c(Cl)ccc2c1-c1cn[nH]c1] |
| 107 | 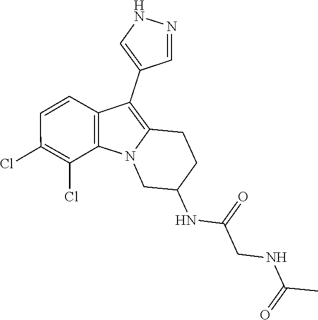 [SMILES: CC(=O)NCC(=O)NC1CCc2c(-c3cn[nH]c3)c3ccc(Cl)c(Cl)c3n2C1] |
| 108 | 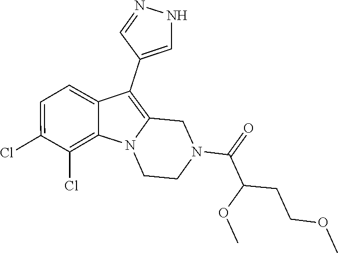 [SMILES: COCCC(OC)C(=O)N1CCn2c(c(-c3cn[nH]c3)c3ccc(Cl)c(Cl)c32)C1] |
| 109 | 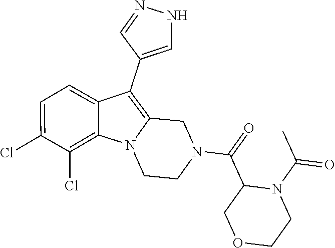 [SMILES: CC(=O)N1CCOCC1C(=O)N1CCn2c(c(-c3cn[nH]c3)c3ccc(Cl)c(Cl)c32)C1] |
| 110 | 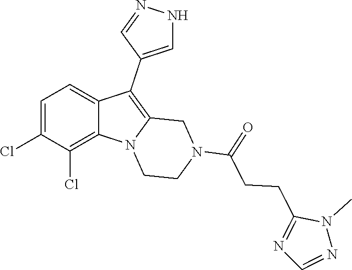 [SMILES: Cn1ncnc1CCC(=O)N1CCn2c(c(-c3cn[nH]c3)c3ccc(Cl)c(Cl)c32)C1] |
| 111 | 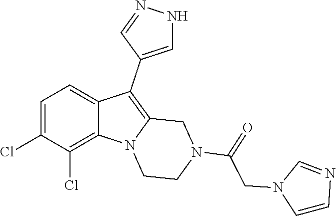 [SMILES: O=C(Cn1ccnc1)N1CCn2c(c(-c3cn[nH]c3)c3ccc(Cl)c(Cl)c32)C1] |
| 112 | 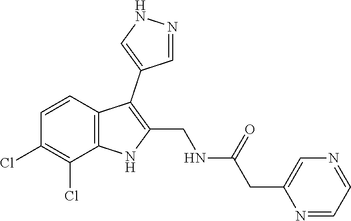 [SMILES: O=C(Cc1cnccn1)NCc1[nH]c2c(Cl)c(Cl)ccc2c1-c1cn[nH]c1] |
| 113 | 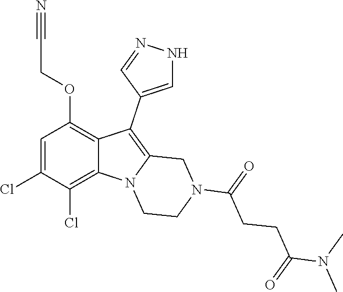 [SMILES: CN(C)C(=O)CCC(=O)N1CCn2c(c(-c3cn[nH]c3)c3c(OCC#N)cc(Cl)c(Cl)c32)C1] |
| 114 | 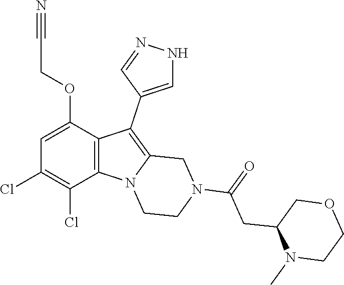 [SMILES: CN1CCOC[C@@H]1CC(=O)N1CCn2c(c(-c3cn[nH]c3)c3c(OCC#N)cc(Cl)c(Cl)c32)C1] |
| 115 | 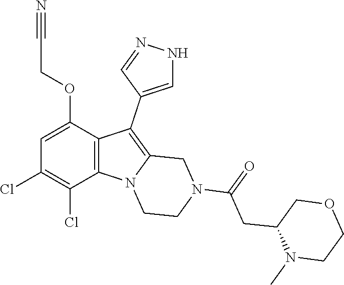 [SMILES: CN1CCOC[C@H]1CC(=O)N1CCn2c(c(-c3cn[nH]c3)c3c(OCC#N)cc(Cl)c(Cl)c32)C1] |
| 116 | 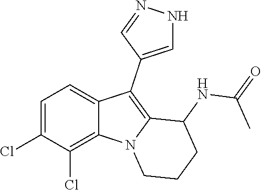 [SMILES: CC(=O)NC1CCCn2c1c(-c1cn[nH]c1)c1ccc(Cl)c(Cl)c12] |
| 117 | 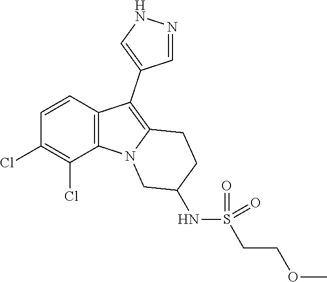 [SMILES: COCCS(=O)(=O)NC1CCc2c(-c3cn[nH]c3)c3ccc(Cl)c(Cl)c3n2C1] |
| 118 | 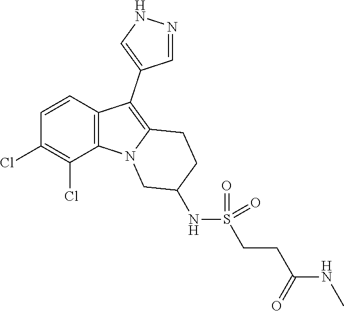 [SMILES: CNC(=O)CCS(=O)(=O)NC1CCc2c(-c3cn[nH]c3)c3ccc(Cl)c(Cl)c3n2C1] |
| 119 | 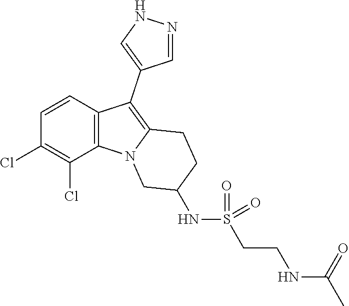 [SMILES: CC(=O)NCCS(=O)(=O)NC1CCc2c(-c3cn[nH]c3)c3ccc(Cl)c(Cl)c3n2C1] |
| 120 | 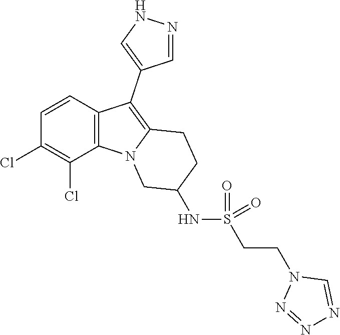 [SMILES: O=S(=O)(CCn1cnnn1)NC1CCc2c(-c3cn[nH]c3)c3ccc(Cl)c(Cl)c3n2C1] |
| 121 | 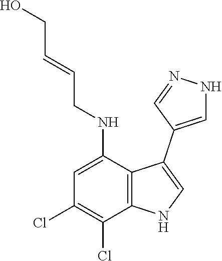 [SMILES: OC/C=C/CNc1cc(Cl)c(Cl)c2[nH]cc(-c3cn[nH]c3)c12] |
| 122 | 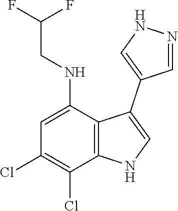 [SMILES: FC(F)CNc1cc(Cl)c(Cl)c2[nH]cc(-c3cn[nH]c3)c12] |
| 123 | 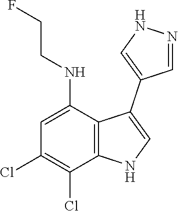 [SMILES: FCCNc1cc(Cl)c(Cl)c2[nH]cc(-c3cn[nH]c3)c12] |
| 124 | 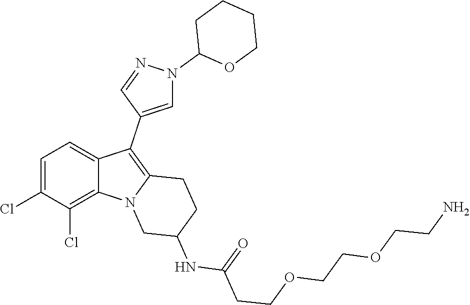 [SMILES: NCCOCCOCCC(=O)NC1CCc2c(-c3cnn(C4CCCCO4)c3)c3ccc(Cl)c(Cl)c3n2C1] |
| 125 | 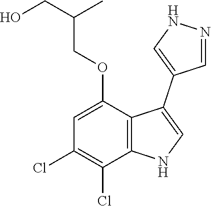 [SMILES: CC(CO)COc1cc(Cl)c(Cl)c2[nH]cc(-c3cn[nH]c3)c12] |
| 126 | 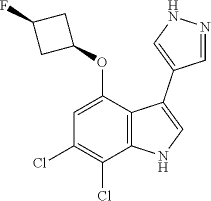 [SMILES: F[C@H]1C[C@@H](Oc2cc(Cl)c(Cl)c3[nH]cc(-c4cn[nH]c4)c23)C1] |
| 127 | 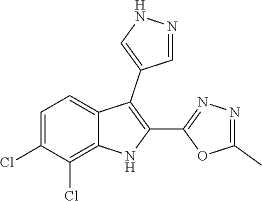 [SMILES: Cc1nnc(-c2[nH]c3c(Cl)c(Cl)ccc3c2-c2cn[nH]c2)o1] |
| 128 | 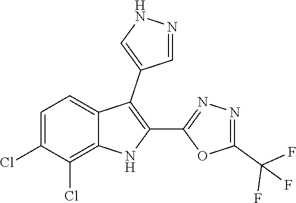 [SMILES: FC(F)(F)c1nnc(-c2[nH]c3c(Cl)c(Cl)ccc3c2-c2cn[nH]c2)o1] |
| 129 | 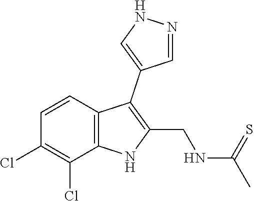 [SMILES: CC(=S)NCc1[nH]c2c(Cl)c(Cl)ccc2c1-c1cn[nH]c1] |
| 130 | 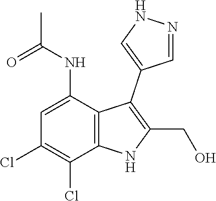 [SMILES: CC(=O)Nc1cc(Cl)c(Cl)c2[nH]c(CO)c(-c3cn[nH]c3)c12] |
| 131 | 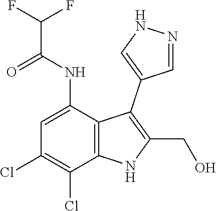 [SMILES: O=C(Nc1cc(Cl)c(Cl)c2[nH]c(CO)c(-c3cn[nH]c3)c12)C(F)F] |
| 132 | 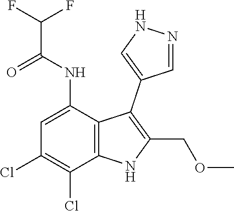 [SMILES: COCc1[nH]c2c(Cl)c(Cl)cc(NC(=O)C(F)F)c2c1-c1cn[nH]c1] |
| 133 | 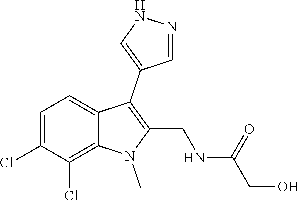 [SMILES: Cn1c(CNC(=O)CO)c(-c2cn[nH]c2)c2ccc(Cl)c(Cl)c21] |
| 134 | 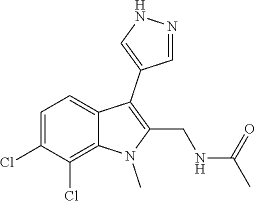 [SMILES: CC(=O)NCc1c(-c2cn[nH]c2)c2ccc(Cl)c(Cl)c2n1C] |
| 135 | 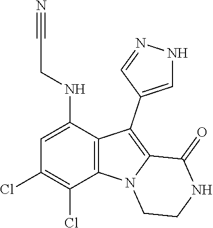 [SMILES: N#CCNc1cc(Cl)c(Cl)c2c1c(-c1cn[nH]c1)c1n2CCNC1=O] |
| 136 | 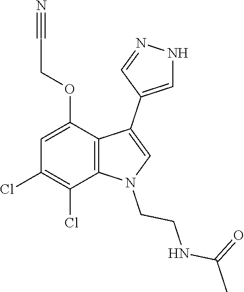 [SMILES: CC(=O)NCCn1cc(-c2cn[nH]c2)c2c(OCC#N)cc(Cl)c(Cl)c21] |
| 137 | 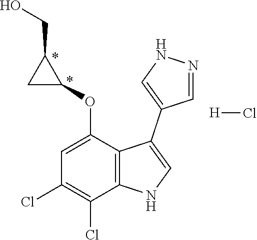 |
| 138 | 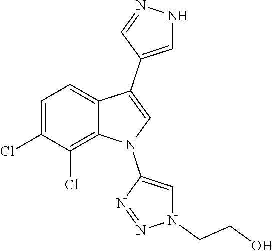 [SMILES: OCCn1cc(-n2cc(-c3cn[nH]c3)c3ccc(Cl)c(Cl)c32)nn1] |
| 139 | 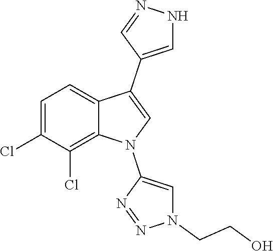 [SMILES: OCCn1cc(-n2cc(-c3cn[nH]c3)c3ccc(Cl)c(Cl)c32)nn1] |
| 140 | 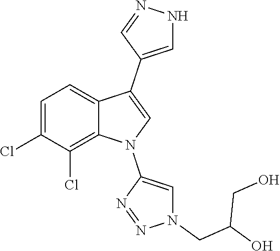 [SMILES: OCC(O)Cn1cc(-n2cc(-c3cn[nH]c3)c3ccc(Cl)c(Cl)c32)nn1] |
| 141 | 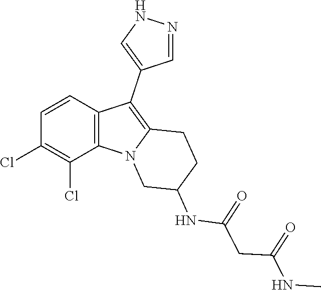 [SMILES: CNC(=O)CC(=O)NC1CCc2c(-c3cn[nH]c3)c3ccc(Cl)c(Cl)c3n2C1] |
| 142 | 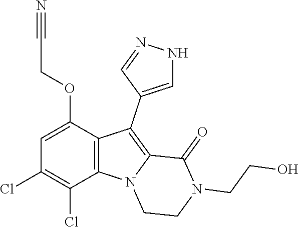 [SMILES: N#CCOc1cc(Cl)c(Cl)c2c1c(-c1cn[nH]c1)c1n2CCN(CCO)C1=O] |
| 143 | 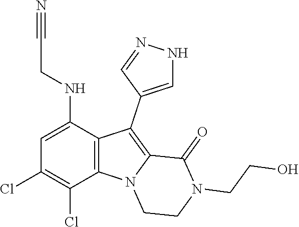 [SMILES: N#CCNc1cc(Cl)c(Cl)c2c1c(-c1cn[nH]c1)c1n2CCN(CCO)C1=O] |
| 144 | 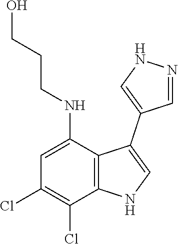 [SMILES: OCCCNc1cc(Cl)c(Cl)c2[nH]cc(-c3cn[nH]c3)c12] |
| 145 | 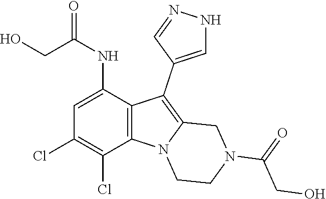 [SMILES: O=C(CO)Nc1cc(Cl)c(Cl)c2c1c(-c1cn[nH]c1)c1n2CCN(C(=O)CO)C1] |
| 146 | 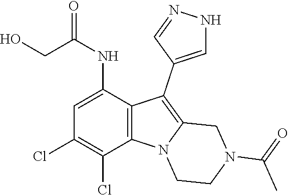 [SMILES: CC(=O)N1CCn2c(c(-c3cn[nH]c3)c3c(NC(=O)CO)cc(Cl)c(Cl)c32)C1] |
| 147 | 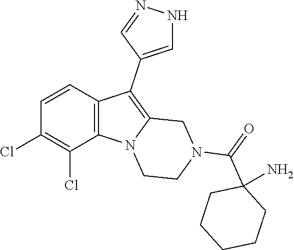 [SMILES: NC1(C(=O)N2CCn3c(c(-c4cn[nH]c4)c4ccc(Cl)c(Cl)c43)C2)CCCCC1] |
| 148 | 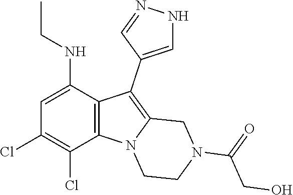 [SMILES: CCNc1cc(Cl)c(Cl)c2c1c(-c1cn[nH]c1)c1n2CCN(C(=O)CO)C1] |
| 149 | 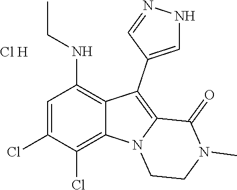 |
| 150 | 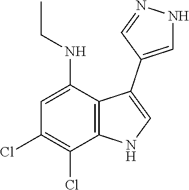 [SMILES: CCNc1cc(Cl)c(Cl)c2[nH]cc(-c3cn[nH]c3)c12] |
| 151 | 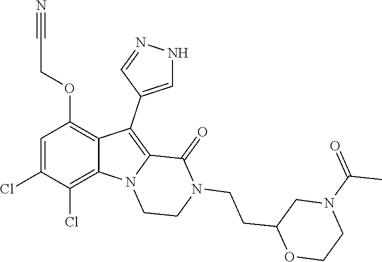 [SMILES: CC(=O)N1CCOC(CCN2CCn3c(c(-c4cn[nH]c4)c4c(OCC#N)cc(Cl)c(Cl)c43)C2=O)C1] |
| 152 | 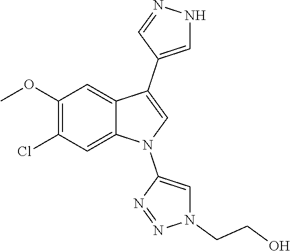 [SMILES: COc1cc2c(-c3cn[nH]c3)cn(-c3cn(CCO)nn3)c2cc1Cl] |
| 153 | 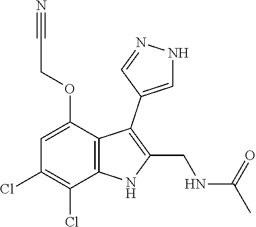 [SMILES: CC(=O)NCc1[nH]c2c(Cl)c(Cl)cc(OCC#N)c2c1-c1cn[nH]c1] |
| 154 | 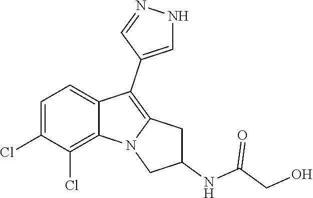 [SMILES: O=C(CO)NC1Cc2c(-c3cn[nH]c3)c3ccc(Cl)c(Cl)c3n2C1] |
| 155 | 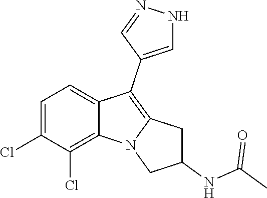 [SMILES: CC(=O)NC1Cc2c(-c3cn[nH]c3)c3ccc(Cl)c(Cl)c3n2C1] |
| 156 | 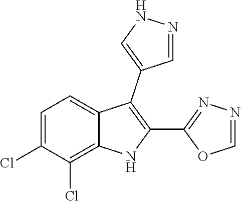 [SMILES: Clc1ccc2c(-c3cn[nH]c3)c(-c3nnco3)[nH]c2c1Cl] |
| 157 | 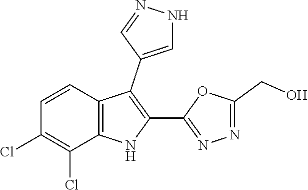 [SMILES: OCc1nnc(-c2[nH]c3c(Cl)c(Cl)ccc3c2-c2cn[nH]c2)o1] |
| 158 | 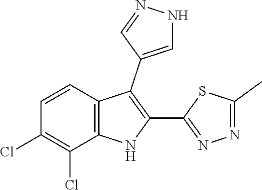 [SMILES: Cc1nnc(-c2[nH]c3c(Cl)c(Cl)ccc3c2-c2cn[nH]c2)s1] |
| 159 | 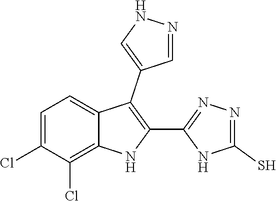 [SMILES: Sc1nnc(-c2[nH]c3c(Cl)c(Cl)ccc3c2-c2cn[nH]c2)[nH]1] |
| 160 | 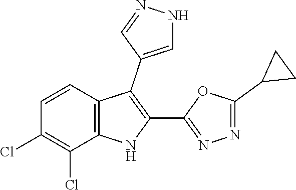 [SMILES: Clc1ccc2c(-c3cn[nH]c3)c(-c3nnc(C4CC4)o3)[nH]c2c1Cl] |
| 161 | 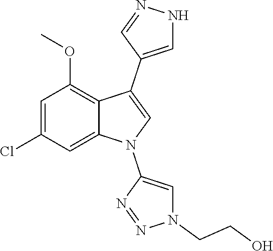 [SMILES: COc1cc(Cl)cc2c1c(-c1cn[nH]c1)cn2-c1cn(CCO)nn1] |
| 162 | 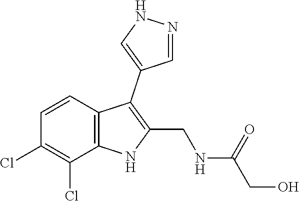 [SMILES: O=C(CO)NCc1[nH]c2c(Cl)c(Cl)ccc2c1-c1cn[nH]c1] |
| 163 | 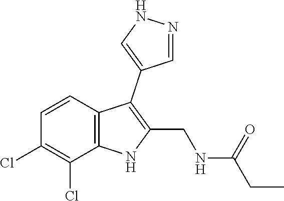 [SMILES: CCC(=O)NCc1[nH]c2c(Cl)c(Cl)ccc2c1-c1cn[nH]c1] |
| 164 | 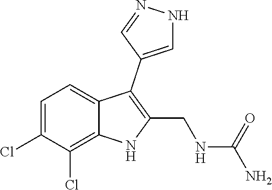 [SMILES: NC(=O)NCc1[nH]c2c(Cl)c(Cl)ccc2c1-c1cn[nH]c1] |
| 165 | 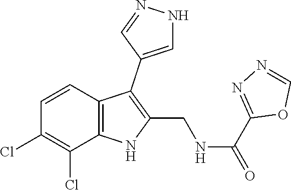 [SMILES: O=C(NCc1[nH]c2c(Cl)c(Cl)ccc2c1-c1cn[nH]c1)c1nnco1] |
| 166 | 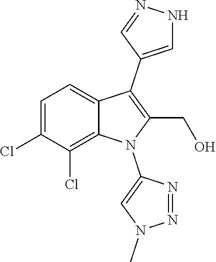 [SMILES: Cn1cc(-n2c(CO)c(-c3cn[nH]c3)c3ccc(Cl)c(Cl)c32)nn1] |
| 167 | 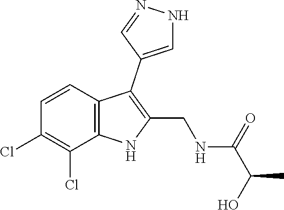 [SMILES: C[C@@H](O)C(=O)NCc1[nH]c2c(Cl)c(Cl)ccc2c1-c1cn[nH]c1] |
| 168 | 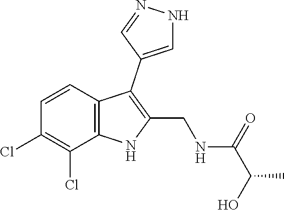 [SMILES: C[C@H](O)C(=O)NCc1[nH]c2c(Cl)c(Cl)ccc2c1-c1cn[nH]c1] |
| 169 | 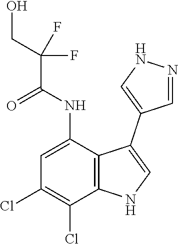 [SMILES: O=C(Nc1cc(Cl)c(Cl)c2[nH]cc(-c3cn[nH]c3)c12)C(F)(F)CO] |
| 170 | 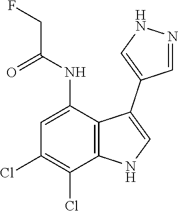 [SMILES: O=C(CF)Nc1cc(Cl)c(Cl)c2[nH]cc(-c3cn[nH]c3)c12] |
| 171 | 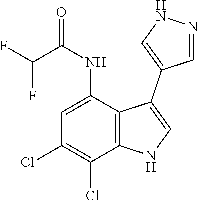 [SMILES: O=C(Nc1cc(Cl)c(Cl)c2[nH]cc(-c3cn[nH]c3)c12)C(F)F] |
| 172 | 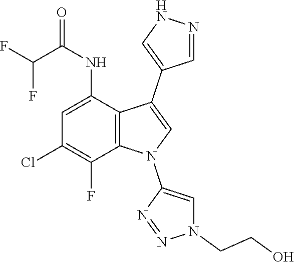 [SMILES: O=C(Nc1cc(Cl)c(F)c2c1c(-c1cn[nH]c1)cn2-c1cn(CCO)nn1)C(F)F] |
| 173 | 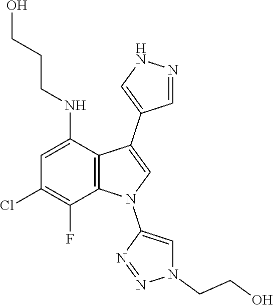 [SMILES: OCCCNc1cc(Cl)c(F)c2c1c(-c1cn[nH]c1)cn2-c1cn(CCO)nn1] |
| 175 | 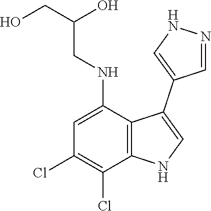 [SMILES: OCC(O)CNc1cc(Cl)c(Cl)c2[nH]cc(-c3cn[nH]c3)c12] |
| 176 | 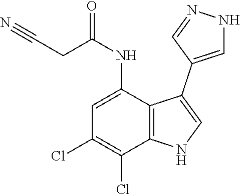 [SMILES: N#CCC(=O)Nc1cc(Cl)c(Cl)c2[nH]cc(-c3cn[nH]c3)c12] |
| 177 | 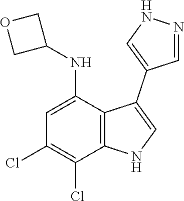 [SMILES: Clc1cc(NC2COC2)c2c(-c3cn[nH]c3)c[nH]c2c1Cl] |
| 178 | 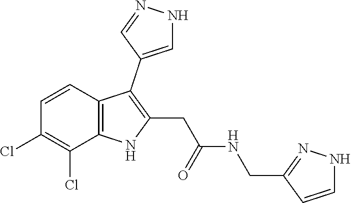 [SMILES: O=C(Cc1[nH]c2c(Cl)c(Cl)ccc2c1-c1cn[nH]c1)NCc1cc[nH]n1] |
| 179 | 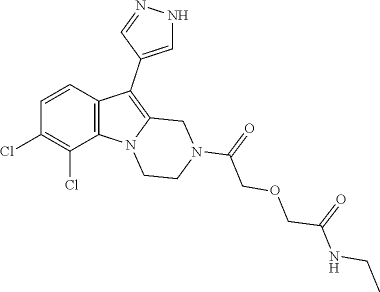 [SMILES: CCNC(=O)COCC(=O)N1CCn2c(c(-c3cn[nH]c3)c3ccc(Cl)c(Cl)c32)C1] |
| 180 | 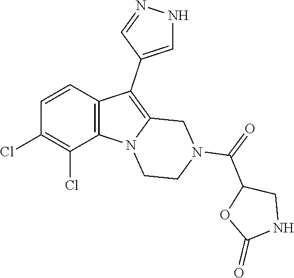 [SMILES: O=C1NCC(C(=O)N2CCn3c(c(-c4cn[nH]c4)c4ccc(Cl)c(Cl)c43)C2)O1] |
| 181 | 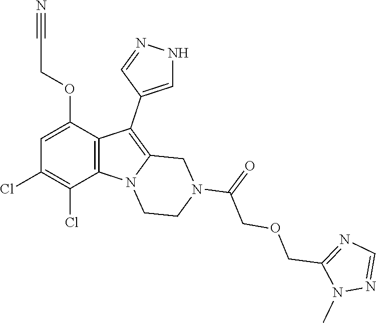 [SMILES: Cn1ncnc1COCC(=O)N1CCn2c(c(-c3cn[nH]c3)c3c(OCC#N)cc(Cl)c(Cl)c32)C1] |
| 182 | 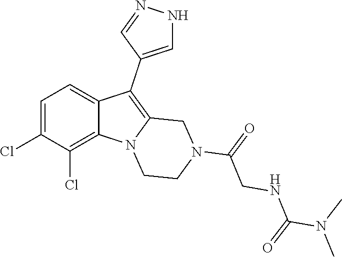 [SMILES: CN(C)C(=O)NCC(=O)N1CCn2c(c(-c3cn[nH]c3)c3ccc(Cl)c(Cl)c32)C1] |
| 183 | 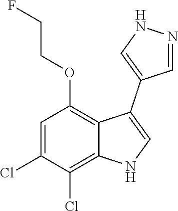 [SMILES: FCCOc1cc(Cl)c(Cl)c2[nH]cc(-c3cn[nH]c3)c12] |
| 184 | 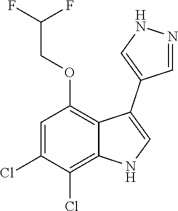 [SMILES: FC(F)COc1cc(Cl)c(Cl)c2[nH]cc(-c3cn[nH]c3)c12] |
| 185 | 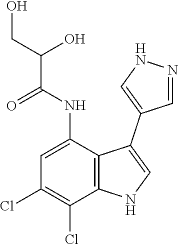 [SMILES: O=C(Nc1cc(Cl)c(Cl)c2[nH]cc(-c3cn[nH]c3)c12)C(O)CO] |
| 186 | 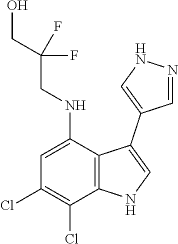 [SMILES: OCC(F)(F)CNc1cc(Cl)c(Cl)c2[nH]cc(-c3cn[nH]c3)c12] |
| 187 | 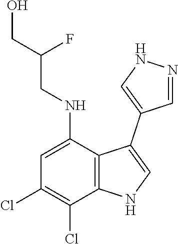 [SMILES: OCC(F)CNc1cc(Cl)c(Cl)c2[nH]cc(-c3cn[nH]c3)c12] |
| 188 | 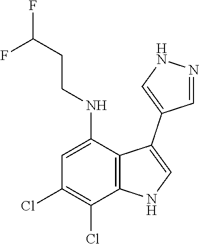 [SMILES: FC(F)CCNc1cc(Cl)c(Cl)c2[nH]cc(-c3cn[nH]c3)c12] |
| 189 | 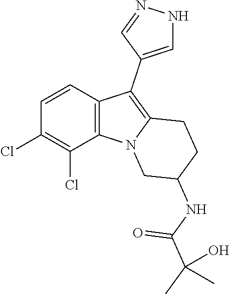 [SMILES: CC(C)(O)C(=O)NC1CCc2c(-c3cn[nH]c3)c3ccc(Cl)c(Cl)c3n2C1] |
| 190 | 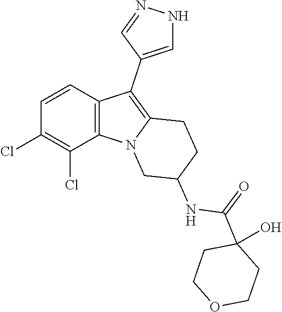 [SMILES: O=C(NC1CCc2c(-c3cn[nH]c3)c3ccc(Cl)c(Cl)c3n2C1)C1(O)CCOCC1] |
| 191 | 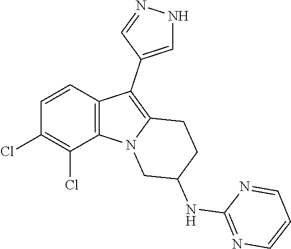 [SMILES: Clc1ccc2c(-c3cn[nH]c3)c3n(c2c1Cl)CC(Nc1ncccn1)CC3] |
| 192 | 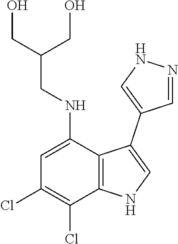 [SMILES: OCC(CO)CNc1cc(Cl)c(Cl)c2[nH]cc(-c3cn[nH]c3)c12] |
| 193 | 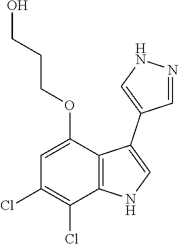 [SMILES: OCCCOc1cc(Cl)c(Cl)c2[nH]cc(-c3cn[nH]c3)c12] |
| 194 | 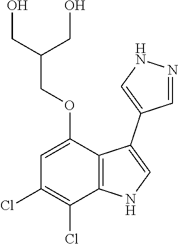 [SMILES: OCC(CO)COc1cc(Cl)c(Cl)c2[nH]cc(-c3cn[nH]c3)c12] |
| 195 | 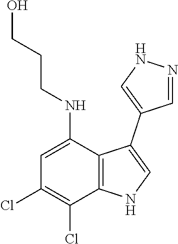 [SMILES: OCCCNc1cc(Cl)c(Cl)c2[nH]cc(-c3cn[nH]c3)c12] |
| 196 | 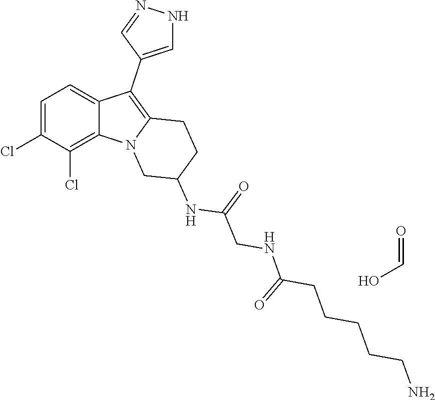 |
| 197 | 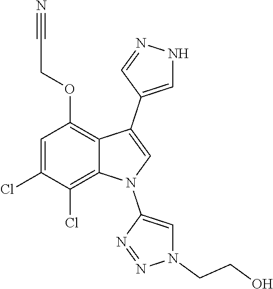 [SMILES: N#CCOc1cc(Cl)c(Cl)c2c1c(-c1cn[nH]c1)cn2-c1cn(CCO)nn1] |
| 198 | 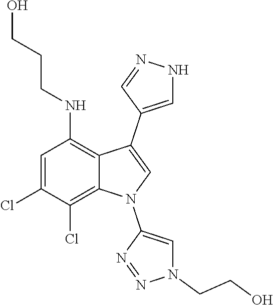 [SMILES: OCCCNc1cc(Cl)c(Cl)c2c1c(-c1cn[nH]c1)cn2-c1cn(CCO)nn1] |
| 199 | 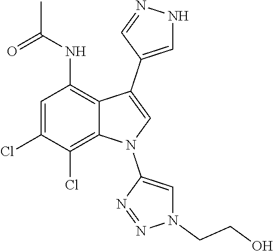 [SMILES: CC(=O)Nc1cc(Cl)c(Cl)c2c1c(-c1cn[nH]c1)cn2-c1cn(CCO)nn1] |
| 200 | 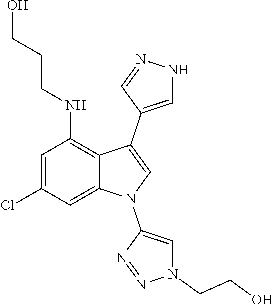 [SMILES: OCCCNc1cc(Cl)cc2c1c(-c1cn[nH]c1)cn2-c1cn(CCO)nn1] |
| 201 | 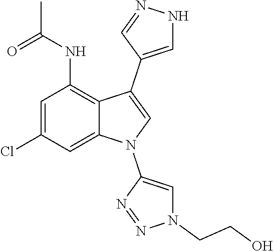 [SMILES: CC(=O)Nc1cc(Cl)cc2c1c(-c1cn[nH]c1)cn2-c1cn(CCO)nn1] |
| 202 | 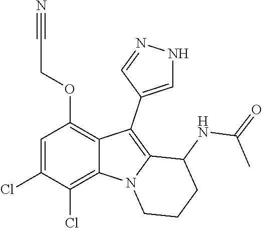 [SMILES: CC(=O)NC1CCCn2c1c(-c1cn[nH]c1)c1c(OCC#N)cc(Cl)c(Cl)c12] |
| 203 | 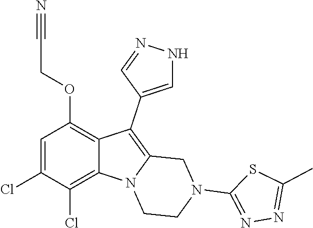 [SMILES: Cc1nnc(N2CCn3c(c(-c4cn[nH]c4)c4c(OCC#N)cc(Cl)c(Cl)c43)C2)s1] |
| 204 | 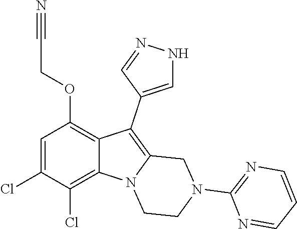 [SMILES: N#CCOc1cc(Cl)c(Cl)c2c1c(-c1cn[nH]c1)c1n2CCN(c2ncccn2)C1] |
| 205 | 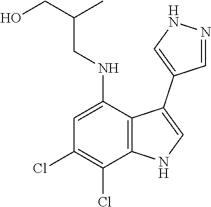 [SMILES: CC(CO)CNc1cc(Cl)c(Cl)c2[nH]cc(-c3cn[nH]c3)c12] |
| 206 | 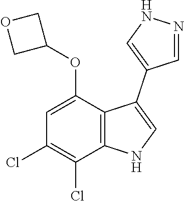 [SMILES: Clc1cc(OC2COC2)c2c(-c3cn[nH]c3)c[nH]c2c1Cl] |
| 207 | 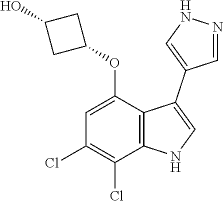 [SMILES: O[C@H]1C[C@@H](Oc2cc(Cl)c(Cl)c3[nH]cc(-c4cn[nH]c4)c23)C1] |
| 208 | 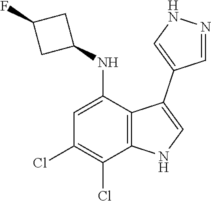 [SMILES: F[C@H]1C[C@@H](Nc2cc(Cl)c(Cl)c3[nH]cc(-c4cn[nH]c4)c23)C1] |
| 209 | 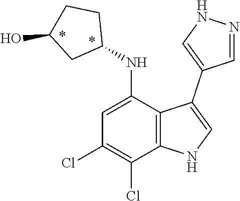 [SMILES: O[C@H]1CC[C@H](Nc2cc(Cl)c(Cl)c3[nH]cc(-c4cn[nH]c4)c23)C1] |
| 210 | 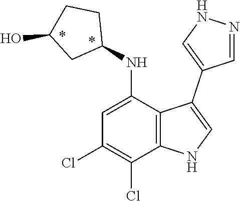 [SMILES: O[C@H]1CC[C@@H](Nc2cc(Cl)c(Cl)c3[nH]cc(-c4cn[nH]c4)c23)C1] Isomer 2 |
| 211 | 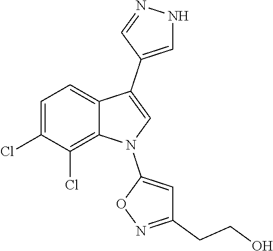 [SMILES: OCCc1cc(-n2cc(-c3cn[nH]c3)c3ccc(Cl)c(Cl)c32)on1] |
| 212 | 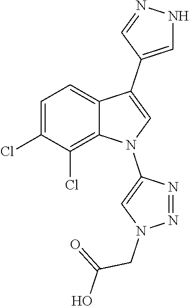 [SMILES: O=C(O)Cn1cc(-n2cc(-c3cn[nH]c3)c3ccc(Cl)c(Cl)c32)nn1] |
| 213 | 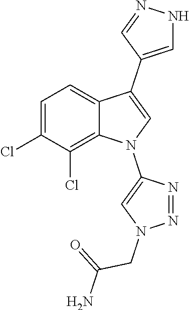 [SMILES: NC(=O)Cn1cc(-n2cc(-c3cn[nH]c3)c3ccc(Cl)c(Cl)c32)nn1] |
| 214 | 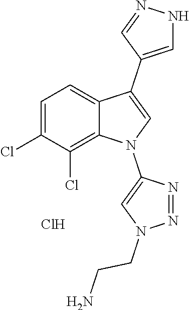 [SMILES: NCCn1cc(-n2cc(-c3cn[nH]c3)c3ccc(Cl)c(Cl)c32)nn1 ; Cl] |
| 215 | 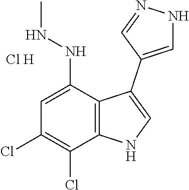 |
| 216 | 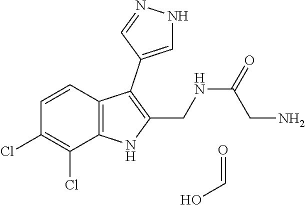 |
| 217 | 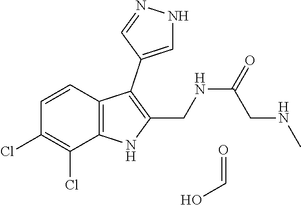 |
| 218 | 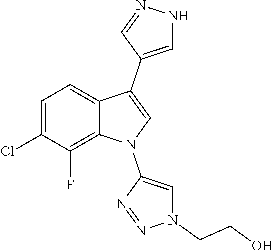 [SMILES: OCCn1cc(-n2cc(-c3cn[nH]c3)c3ccc(Cl)c(F)c32)nn1] |
| 219 | 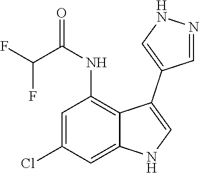 [SMILES: O=C(Nc1cc(Cl)cc2[nH]cc(-c3cn[nH]c3)c12)C(F)F] |
| 220 | 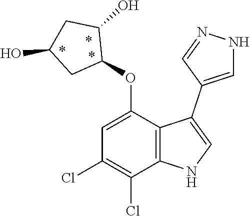 [SMILES: O[C@@H]1C[C@H](Oc2cc(Cl)c(Cl)c3[nH]cc(-c4cn[nH]c4)c23)[C@@H](O)C1] |
| 221 | 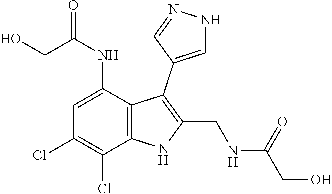 [SMILES: O=C(CO)NCc1[nH]c2c(Cl)c(Cl)cc(NC(=O)CO)c2c1-c1cn[nH]c1] |
| 222 | 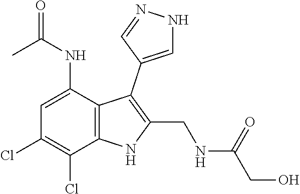 [SMILES: CC(=O)Nc1cc(Cl)c(Cl)c2[nH]c(CNC(=O)CO)c(-c3cn[nH]c3)c12] |
| 223 | 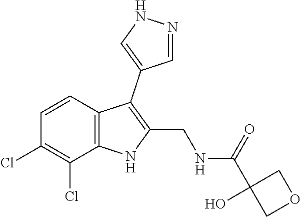 [SMILES: O=C(NCc1[nH]c2c(Cl)c(Cl)ccc2c1-c1cn[nH]c1)C1(O)COC1] |
| 224 | 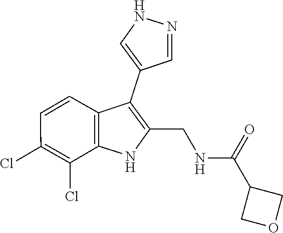 [SMILES: O=C(NCc1[nH]c2c(Cl)c(Cl)ccc2c1-c1cn[nH]c1)C1COC1] |
| 225 | 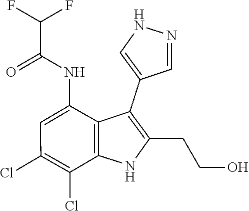 [SMILES: O=C(Nc1cc(Cl)c(Cl)c2[nH]c(CCO)c(-c3cn[nH]c3)c12)C(F)F] |
| 226 | 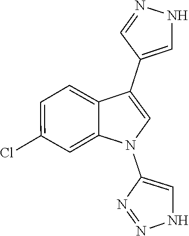 [SMILES: Clc1ccc2c(-c3cn[nH]c3)cn(-c3c[nH]nn3)c2c1] |
| 227 | 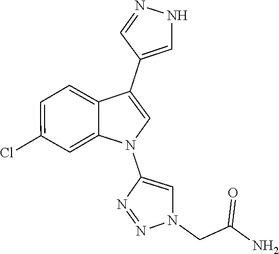 [SMILES: NC(=O)Cn1cc(-n2cc(-c3cn[nH]c3)c3ccc(Cl)cc32)nn1] |
| 228 | 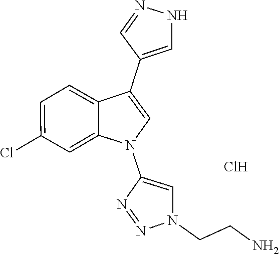 [SMILES: NCCn1cc(-n2cc(-c3cn[nH]c3)c3ccc(Cl)cc32)nn1 ; Cl] |
| 229 | 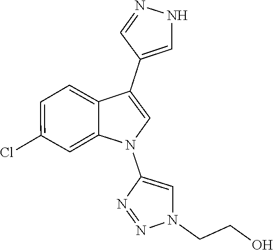 [SMILES: OCCn1cc(-n2cc(-c3cn[nH]c3)c3ccc(Cl)cc32)nn1] |
| 230 | 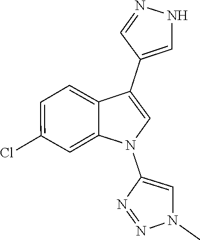 [SMILES: Cn1cc(-n2cc(-c3cn[nH]c3)c3ccc(Cl)cc32)nn1] |
| 231 | 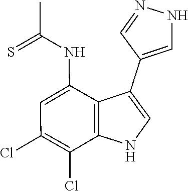 [SMILES: CC(=S)Nc1cc(Cl)c(Cl)c2[nH]cc(-c3cn[nH]c3)c12] |
| 232 | 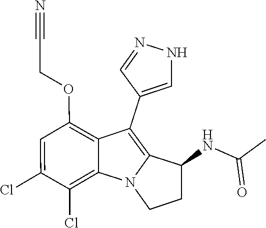 [SMILES: CC(=O)N[C@H]1CCn2c1c(-c1cn[nH]c1)c1c(OCC#N)cc(Cl)c(Cl)c12] |
| 233 | 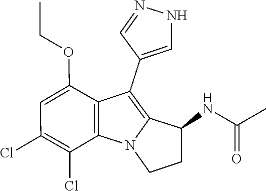 [SMILES: CCOc1cc(Cl)c(Cl)c2c1c(-c1cn[nH]c1)c1n2CC[C@@H]1NC(C)=O] |
| 234 | 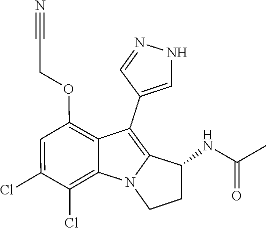 [SMILES: CC(=O)N[C@@H]1CCn2c1c(-c1cn[nH]c1)c1c(OCC#N)cc(Cl)c(Cl)c12] |
| 235 | 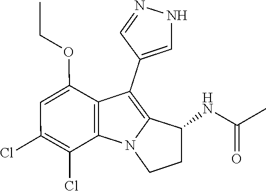 [SMILES: CCOc1cc(Cl)c(Cl)c2c1c(-c1cn[nH]c1)c1n2CC[C@H]1NC(C)=O] |
| 236 | 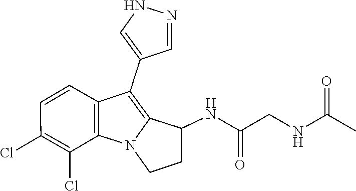 [SMILES: CC(=O)NCC(=O)NC1CCn2c1c(-c1cn[nH]c1)c1ccc(Cl)c(Cl)c12] |
| 237 | 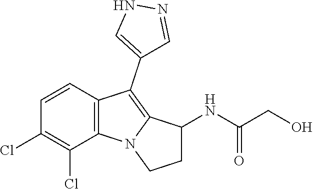 [SMILES: O=C(CO)NC1CCn2c1c(-c1cn[nH]c1)c1ccc(Cl)c(Cl)c12] |
| 238 | 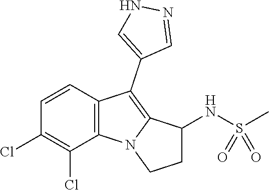 [SMILES: CS(=O)(=O)NC1CCn2c1c(-c1cn[nH]c1)c1ccc(Cl)c(Cl)c12] |
| 239 | 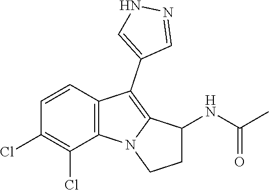 [SMILES: CC(=O)NC1CCn2c1c(-c1cn[nH]c1)c1ccc(Cl)c(Cl)c12] |
| 240 | 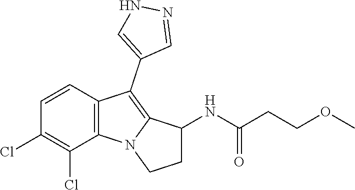 [SMILES: COCCC(=O)NC1CCn2c1c(-c1cn[nH]c1)c1ccc(Cl)c(Cl)c12] |
| 241 | 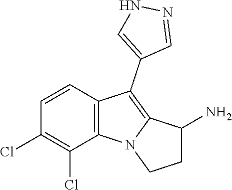 [SMILES: NC1CCn2c1c(-c1cn[nH]c1)c1ccc(Cl)c(Cl)c12] |
| 242 | 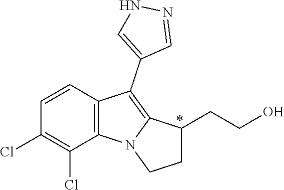 [SMILES: OCCC1CCn2c1c(-c1cn[nH]c1)c1ccc(Cl)c(Cl)c12] Isomer 1 |
| 243 | 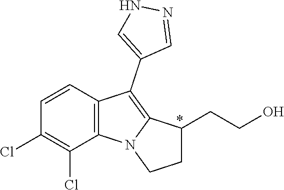 [SMILES: OCCC1CCn2c1c(-c1cn[nH]c1)c1ccc(Cl)c(Cl)c12] Isomer 2 |
| 244 | 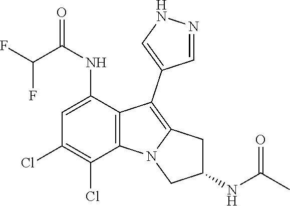 [SMILES: CC(=O)N[C@H]1Cc2c(-c3cn[nH]c3)c3c(NC(=O)C(F)F)cc(Cl)c(Cl)c3n2C1] |
| 245 | 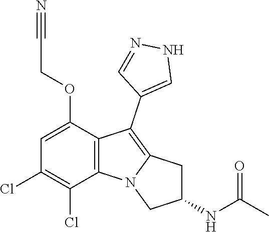 [SMILES: CC(=O)N[C@H]1Cc2c(-c3cn[nH]c3)c3c(OCC#N)cc(Cl)c(Cl)c3n2C1] |
| 246 | 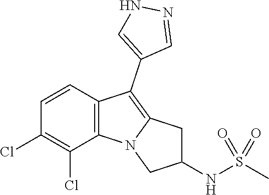 [SMILES: CS(=O)(=O)NC1Cc2c(-c3cn[nH]c3)c3ccc(Cl)c(Cl)c3n2C1] |
| 247 | 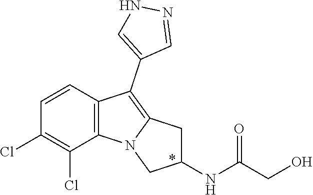 [SMILES: O=C(CO)NC1Cc2c(-c3cn[nH]c3)c3ccc(Cl)c(Cl)c3n2C1] Isomer 1 |
| 248 | 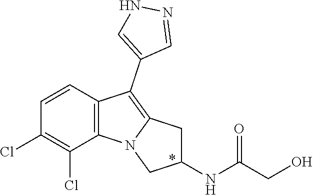 [SMILES: O=C(CO)NC1Cc2c(-c3cn[nH]c3)c3ccc(Cl)c(Cl)c3n2C1] Isomer 2 |
| 249 | 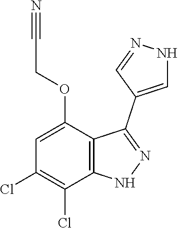 [SMILES: N#CCOc1cc(Cl)c(Cl)c2[nH]nc(-c3cn[nH]c3)c12] |
| 250 | 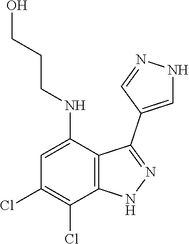 [SMILES: OCCCNc1cc(Cl)c(Cl)c2[nH]nc(-c3cn[nH]c3)c12] |
| 251 | 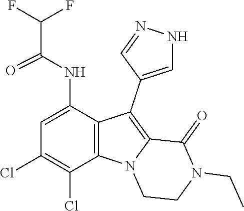 [SMILES: CCN1CCn2c(c(-c3cn[nH]c3)c3c(NC(=O)C(F)F)cc(Cl)c(Cl)c32)C1=O] |
| 252 | 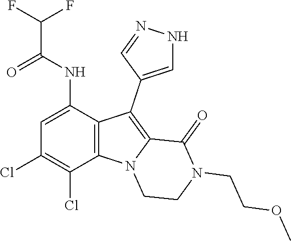 [SMILES: COCCN1CCn2c(c(-c3cn[nH]c3)c3c(NC(=O)C(F)F)cc(Cl)c(Cl)c32)C1=O] |
| 253 | 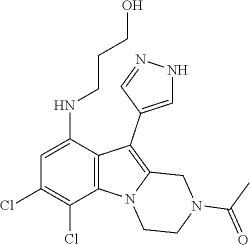 [SMILES: CC(=O)N1CCn2c(c(-c3cn[nH]c3)c3c(NCCCO)cc(Cl)c(Cl)c32)C1] |
| 254 | 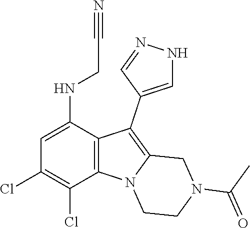 [SMILES: CC(=O)N1CCn2c(c(-c3cn[nH]c3)c3c(NCC#N)cc(Cl)c(Cl)c32)C1] |
| 255 | 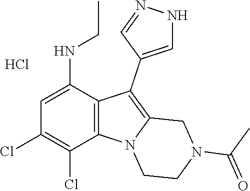 [SMILES: Cl ; CCNc1cc(Cl)c(Cl)c2c1c(-c1cn[nH]c1)c1n2CCN(C(C)=O)C1] |
| 256 | 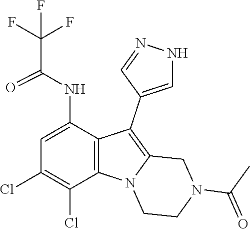 [SMILES: CC(=O)N1CCn2c(c(-c3cn[nH]c3)c3c(NC(=O)C(F)(F)F)cc(Cl)c(Cl)c32)C1] |
| 257 | 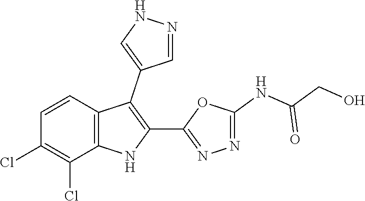 [SMILES: O=C(CO)Nc1nnc(-c2[nH]c3c(Cl)c(Cl)ccc3c2-c2cn[nH]c2)o1] |
| 258 | 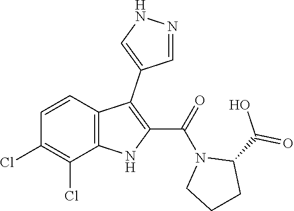 [SMILES: O=C(O)[C@@H]1CCCN1C(=O)c1[nH]c2c(Cl)c(Cl)ccc2c1-c1cn[nH]c1] |
| 259 | 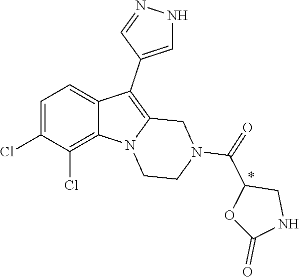 [SMILES: O=C1NCC(C(=O)N2CCn3c(c(-c4cn[nH]c4)c4ccc(Cl)c(Cl)c43)C2)O1] Isomer 1 |
| 260 | 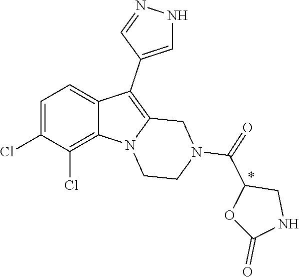 [SMILES: O=C1NCC(C(=O)N2CCn3c(c(-c4cn[nH]c4)c4ccc(Cl)c(Cl)c43)C2)O1] Isomer 2 |
| 261 | 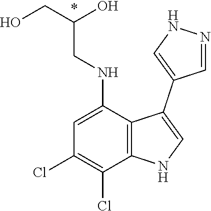 [SMILES: OCC(O)CNc1cc(Cl)c(Cl)c2[nH]cc(-c3cn[nH]c3)c12] Isomer 1 |
| 262 | 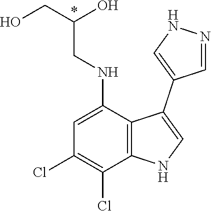 [SMILES: OCC(O)CNc1cc(Cl)c(Cl)c2[nH]cc(-c3cn[nH]c3)c12] Isomer 2 |
| 263 | 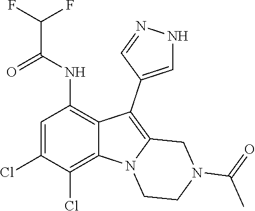 [SMILES: CC(=O)N1CCn2c(c(-c3cn[nH]c3)c3c(NC(=O)C(F)F)cc(Cl)c(Cl)c32)C1] |
| 264 | 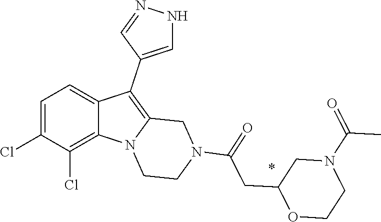 [SMILES: CC(=O)N1CCOC(CC(=O)N2CCn3c(c(-c4cn[nH]c4)c4ccc(Cl)c(Cl)c43)C2)C1] Isomer 1 |
| 265 | 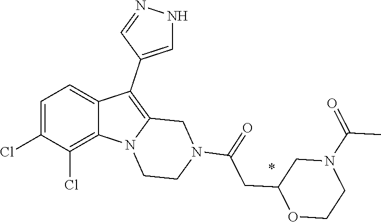 [SMILES: CC(=O)N1CCOC(CC(=O)N2CCn3c(c(-c4cn[nH]c4)c4ccc(Cl)c(Cl)c43)C2)C1] Isomer 2 |
| 266 | 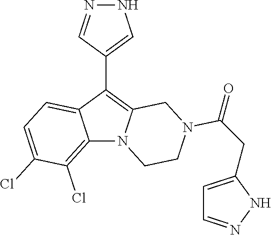 [SMILES: O=C(Cc1ccn[nH]1)N1CCn2c(c(-c3cn[nH]c3)c3ccc(Cl)c(Cl)c32)C1] |
| 267 | 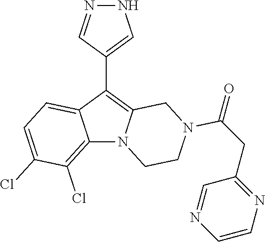 [SMILES: O=C(Cc1cnccn1)N1CCn2c(c(-c3cn[nH]c3)c3ccc(Cl)c(Cl)c32)C1] |
| 268 | 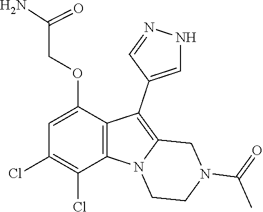 [SMILES: CC(=O)N1CCn2c(c(-c3cn[nH]c3)c3c(OCC(N)=O)cc(Cl)c(Cl)c32)C1] |
| 269 | 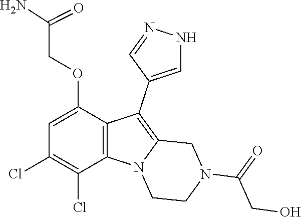 [SMILES: NC(=O)COc1cc(Cl)c(Cl)c2c1c(-c1cn[nH]c1)c1n2CCN(C(=O)CO)C1] |
| 270 | 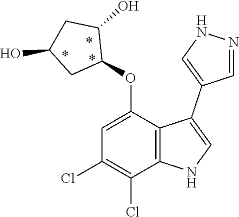 [SMILES: O[C@@H]1C[C@H](Oc2cc(Cl)c(Cl)c3[nH]cc(-c4cn[nH]c4)c23)[C@@H](O)C1] Isomer 1 |
| 271 | 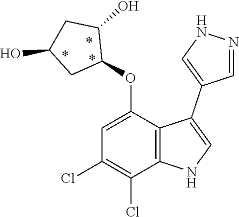 [SMILES: O[C@@H]1C[C@H](Oc2cc(Cl)c(Cl)c3[nH]cc(-c4cn[nH]c4)c23)[C@@H](O)C1] Isomer 2 |
| 272 | 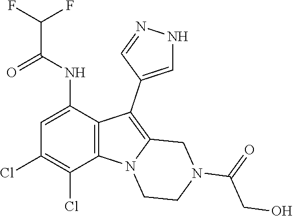 [SMILES: O=C(Nc1cc(Cl)c(Cl)c2c1c(-c1cn[nH]c1)c1n2CCN(C(=O)CO)C1)C(F)F] |
| 273 | 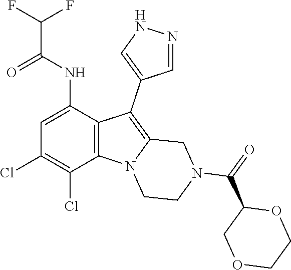 [SMILES: O=C(Nc1cc(Cl)c(Cl)c2c1c(-c1cn[nH]c1)c1n2CCN(C(=O)[C@@H]2COCCO2)C1)C(F)F] |
| 274 | 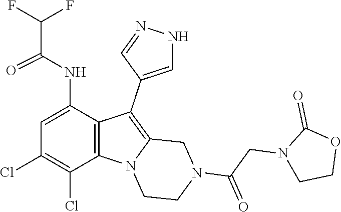 [SMILES: O=C(Nc1cc(Cl)c(Cl)c2c1c(-c1cn[nH]c1)c1n2CCN(C(=O)CN2CCOC2=O)C1)C(F)F] |
| 275 | 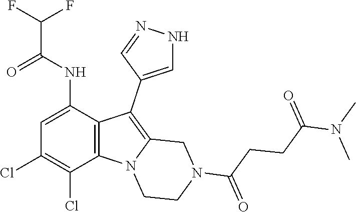 [SMILES: CN(C)C(=O)CCC(=O)N1CCn2c(c(-c3cn[nH]c3)c3c(NC(=O)C(F)F)cc(Cl)c(Cl)c32)C1] |
| 276 | 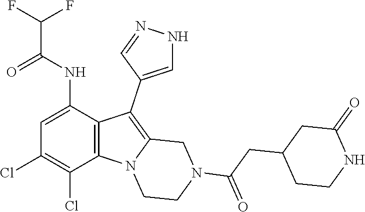 [SMILES: O=C1CC(CC(=O)N2CCn3c(c(-c4cn[nH]c4)c4c(NC(=O)C(F)F)cc(Cl)c(Cl)c43)C2)CCN1] |
| 277 | 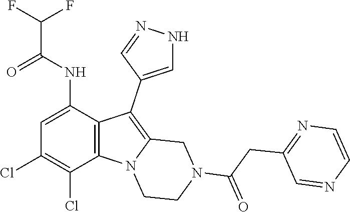 [SMILES: O=C(Nc1cc(Cl)c(Cl)c2c1c(-c1cn[nH]c1)c1n2CCN(C(=O)Cc2cnccn2)C1)C(F)F] |
| 278 | 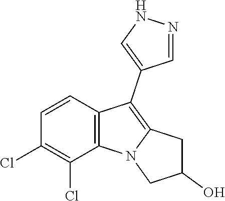 [SMILES: OC1Cc2c(-c3cn[nH]c3)c3ccc(Cl)c(Cl)c3n2C1] |
| 279 | 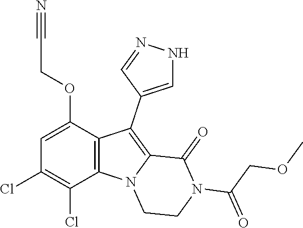 [SMILES: COCC(=O)N1CCn2c(c(-c3cn[nH]c3)c3c(OCC#N)cc(Cl)c(Cl)c32)C1=O] |
| 280 | 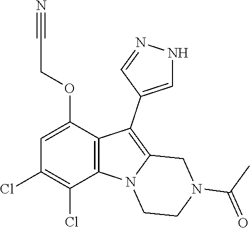 [SMILES: CC(=O)N1CCn2c(c(-c3cn[nH]c3)c3c(OCC#N)cc(Cl)c(Cl)c32)C1] |
| 281 | 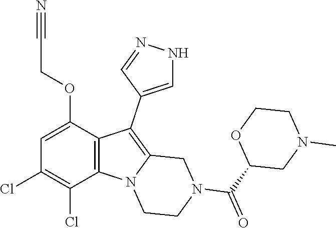 [SMILES: CN1CCO[C@@H](C(=O)N2CCn3c(c(-c4cn[nH]c4)c4c(OCC#N)cc(Cl)c(Cl)c43)C2)C1] |
| 282 | 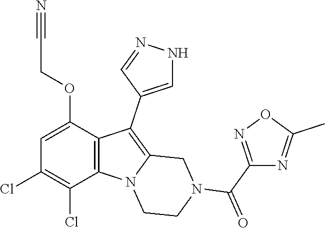 [SMILES: Cc1nc(C(=O)N2CCn3c(c(-c4cn[nH]c4)c4c(OCC#N)cc(Cl)c(Cl)c43)C2)no1] |
| 283 | 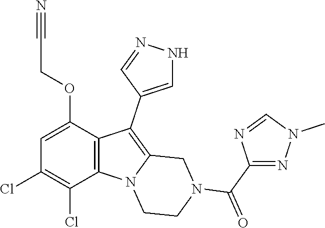 [SMILES: Cn1cnc(C(=O)N2CCn3c(c(-c4cn[nH]c4)c4c(OCC#N)cc(Cl)c(Cl)c43)C2)n1] |
| 284 | 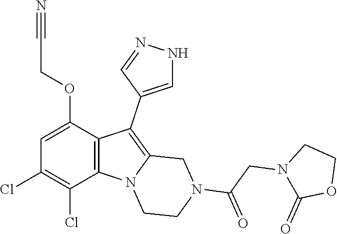 [SMILES: N#CCOc1cc(Cl)c(Cl)c2c1c(-c1cn[nH]c1)c1n2CCN(C(=O)CN2CCOC2=O)C1] |
| 285 | 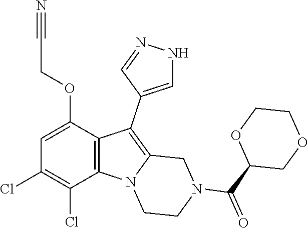 [SMILES: N#CCOc1cc(Cl)c(Cl)c2c1c(-c1cn[nH]c1)c1n2CCN(C(=O)[C@@H]2COCCO2)C1] |
| 286 | 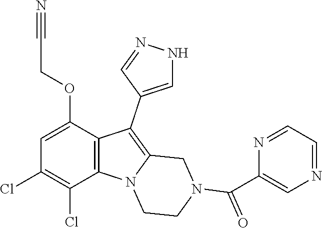 [SMILES: N#CCOc1cc(Cl)c(Cl)c2c1c(-c1cn[nH]c1)c1n2CCN(C(=O)c2cnccn2)C1] |
| 287 | 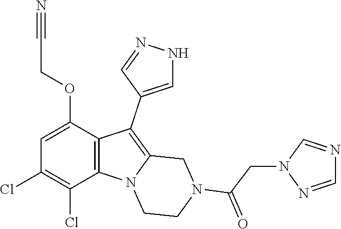 [SMILES: N#CCOc1cc(Cl)c(Cl)c2c1c(-c1cn[nH]c1)c1n2CCN(C(=O)Cn2cncn2)C1] |
| 288 | 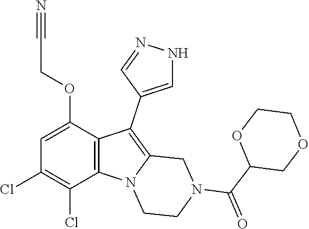 [SMILES: N#CCOc1cc(Cl)c(Cl)c2c1c(-c1cn[nH]c1)c1n2CCN(C(=O)C2COCCO2)C1] |
| 289 | 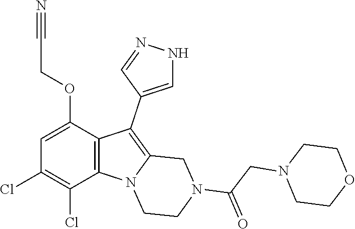 [SMILES: N#CCOc1cc(Cl)c(Cl)c2c1c(-c1cn[nH]c1)c1n2CCN(C(=O)CN2CCOCC2)C1] |
| 290 | 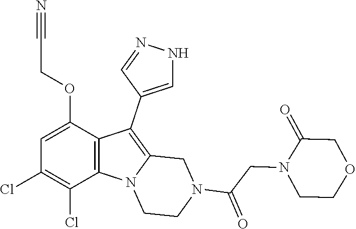 [SMILES: N#CCOc1cc(Cl)c(Cl)c2c1c(-c1cn[nH]c1)c1n2CCN(C(=O)CN2CCOCC2=O)C1] |
| 291 | 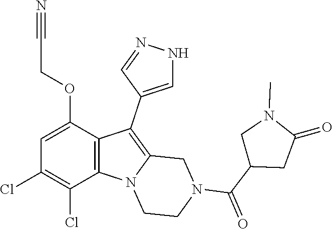 [SMILES: CN1CC(C(=O)N2CCn3c(c(-c4cn[nH]c4)c4c(OCC#N)cc(Cl)c(Cl)c43)C2)CC1=O] |
| 292 | 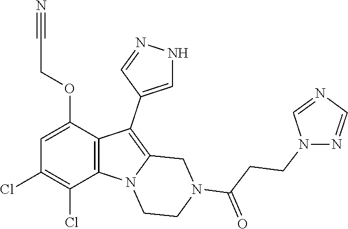 [SMILES: N#CCOc1cc(Cl)c(Cl)c2c1c(-c1cn[nH]c1)c1n2CCN(C(=O)CCn2cncn2)C1] |
| 293 | 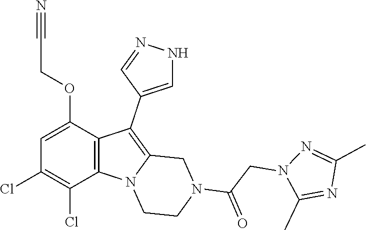 [SMILES: Cc1nc(C)n(CC(=O)N2CCn3c(c(-c4cn[nH]c4)c4c(OCC#N)cc(Cl)c(Cl)c43)C2)n1] |
| 294 | 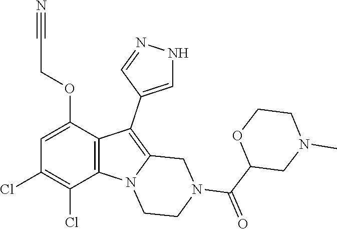 [SMILES: CN1CCOC(C(=O)N2CCn3c(c(-c4cn[nH]c4)c4c(OCC#N)cc(Cl)c(Cl)c43)C2)C1] |
| 295 | 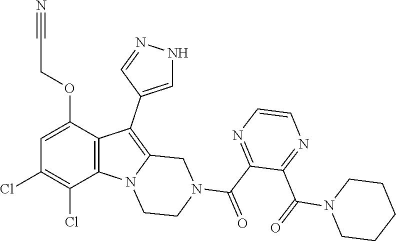 [SMILES: N#CCOc1cc(Cl)c(Cl)c2c1c(-c1cn[nH]c1)c1n2CCN(C(=O)c2nccnc2C(=O)N2CCCCC2)C1] |
| 296 | 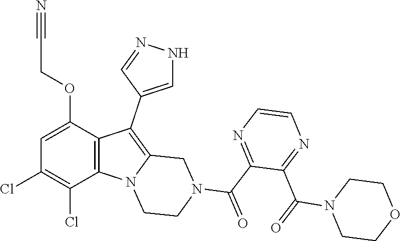 [SMILES: N#CCOc1cc(Cl)c(Cl)c2c1c(-c1cn[nH]c1)c1n2CCN(C(=O)c2nccnc2C(=O)N2CCOCC2)C1] |
| 297 | 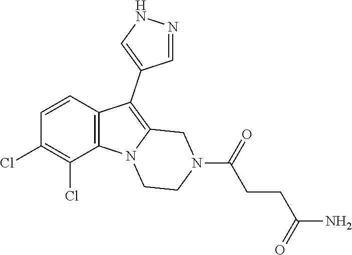 [SMILES: NC(=O)CCC(=O)N1CCn2c(c(-c3cn[nH]c3)c3ccc(Cl)c(Cl)c32)C1] |
| 298 | 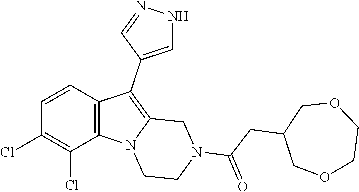 [SMILES: O=C(CC1COCCOC1)N1CCn2c(c(-c3cn[nH]c3)c3ccc(Cl)c(Cl)c32)C1] |
| 299 | 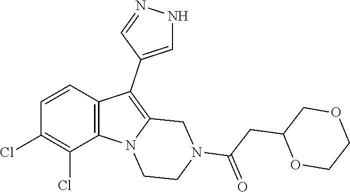 [SMILES: O=C(CC1COCCO1)N1CCn2c(c(-c3cn[nH]c3)c3ccc(Cl)c(Cl)c32)C1] |
| 300 | 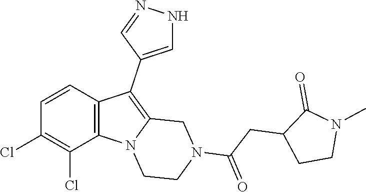 [SMILES: CN1CCC(CC(=O)N2CCn3c(c(-c4cn[nH]c4)c4ccc(Cl)c(Cl)c43)C2)C1=O] |
| 301 | 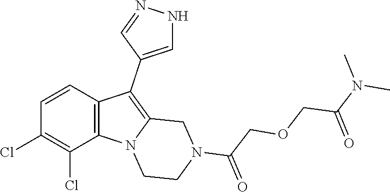 [SMILES: CN(C)C(=O)COCC(=O)N1CCn2c(c(-c3cn[nH]c3)c3ccc(Cl)c(Cl)c32)C1] |
| 302 | 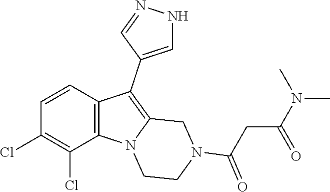 [SMILES: CN(C)C(=O)CC(=O)N1CCn2c(c(-c3cn[nH]c3)c3ccc(Cl)c(Cl)c32)C1] |
| 303 | 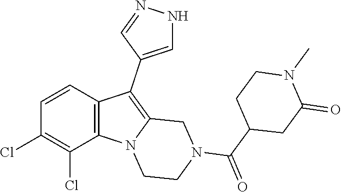 [SMILES: CN1CCC(C(=O)N2CCn3c(c(-c4cn[nH]c4)c4ccc(Cl)c(Cl)c43)C2)CC1=O] |
| 304 | 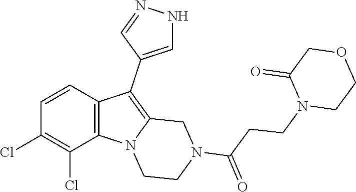 [SMILES: O=C1COCCN1CCC(=O)N1CCn2c(c(-c3cn[nH]c3)c3ccc(Cl)c(Cl)c32)C1] |
| 305 | 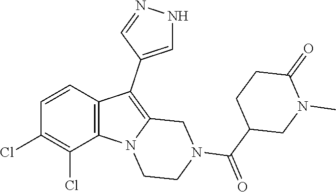 [SMILES: CN1CC(C(=O)N2CCn3c(c(-c4cn[nH]c4)c4ccc(Cl)c(Cl)c43)C2)CCC1=O] |
| 306 | 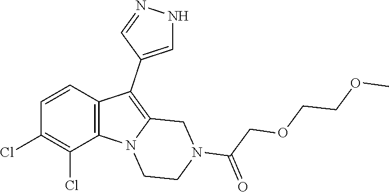 [SMILES: COCCOCC(=O)N1CCn2c(c(-c3cn[nH]c3)c3ccc(Cl)c(Cl)c32)C1] |
| 307 | 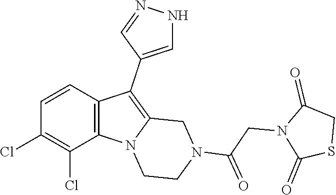 [SMILES: O=C(CN1C(=O)CSC1=O)N1CCn2c(c(-c3cn[nH]c3)c3ccc(Cl)c(Cl)c32)C1] |
| 308 | 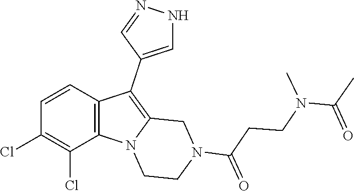 [SMILES: CC(=O)N(C)CCC(=O)N1CCn2c(c(-c3cn[nH]c3)c3ccc(Cl)c(Cl)c32)C1] |
| 309 | 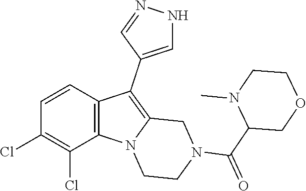 [SMILES: CN1CCOCC1C(=O)N1CCn2c(c(-c3cn[nH]c3)c3ccc(Cl)c(Cl)c32)C1] |
| 310 | 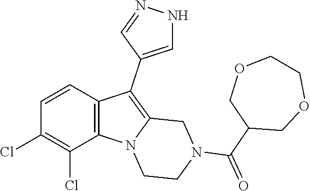 [SMILES: O=C(C1COCCOC1)N1CCn2c(c(-c3cn[nH]c3)c3ccc(Cl)c(Cl)c32)C1] |
| 311 | 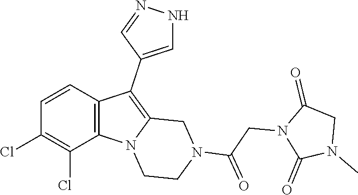 [SMILES: CN1CC(=O)N(CC(=O)N2CCn3c(c(-c4cn[nH]c4)c4ccc(Cl)c(Cl)c43)C2)C1=O] |
| 312 | 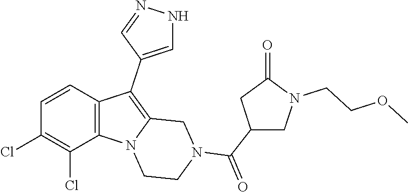 [SMILES: COCCN1CC(C(=O)N2CCn3c(c(-c4cn[nH]c4)c4ccc(Cl)c(Cl)c43)C2)CC1=O] |
| 313 | 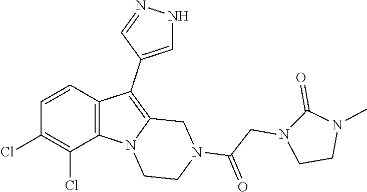 [SMILES: CN1CCN(CC(=O)N2CCn3c(c(-c4cn[nH]c4)c4ccc(Cl)c(Cl)c43)C2)C1=O] |
| 314 | 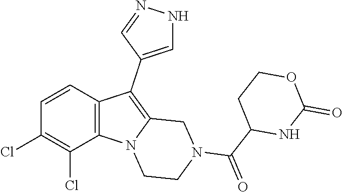 [SMILES: O=C1NC(C(=O)N2CCn3c(c(-c4cn[nH]c4)c4ccc(Cl)c(Cl)c43)C2)CCO1] |
| 315 | 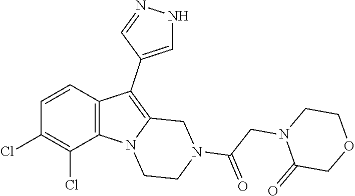 [SMILES: O=C1COCCN1CC(=O)N1CCn2c(c(-c3cn[nH]c3)c3ccc(Cl)c(Cl)c32)C1] |
| 316 | 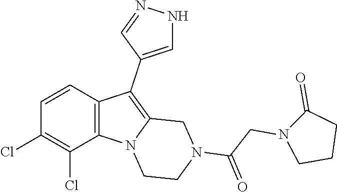 [SMILES: O=C1CCCN1CC(=O)N1CCn2c(c(-c3cn[nH]c3)c3ccc(Cl)c(Cl)c32)C1] |
| 317 | 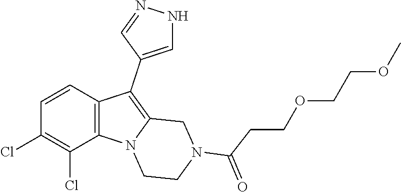 [SMILES: COCCOCCC(=O)N1CCn2c(c(-c3cn[nH]c3)c3ccc(Cl)c(Cl)c32)C1] |
| 318 | 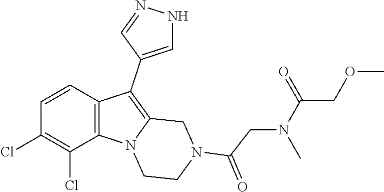 [SMILES: COCC(=O)N(C)CC(=O)N1CCn2c(c(-c3cn[nH]c3)c3ccc(Cl)c(Cl)c32)C1] |
| 319 | 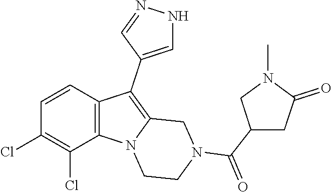 [SMILES: CN1CC(C(=O)N2CCn3c(c(-c4cn[nH]c4)c4ccc(Cl)c(Cl)c43)C2)CC1=O] |
| 320 | 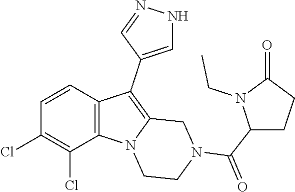 [SMILES: CCN1C(=O)CCC1C(=O)N1CCn2c(c(-c3cn[nH]c3)c3ccc(Cl)c(Cl)c32)C1] |
| 321 | 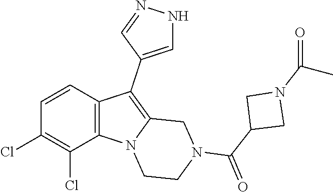 [SMILES: CC(=O)N1CC(C(=O)N2CCn3c(c(-c4cn[nH]c4)c4ccc(Cl)c(Cl)c43)C2)C1] |
| 322 | 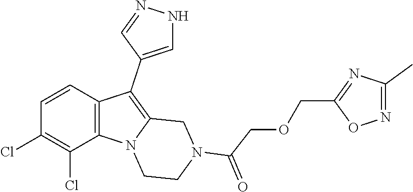 [SMILES: Cc1noc(COCC(=O)N2CCn3c(c(-c4cn[nH]c4)c4ccc(Cl)c(Cl)c43)C2)n1] |
| 323 | 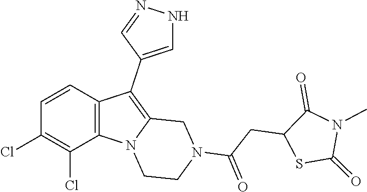 [SMILES: CN1C(=O)SC(CC(=O)N2CCn3c(c(-c4cn[nH]c4)c4ccc(Cl)c(Cl)c43)C2)C1=O] |
| 324 | 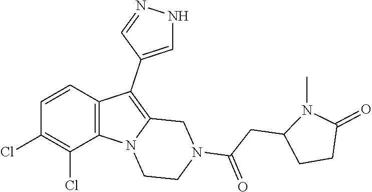 [SMILES: CN1C(=O)CCC1CC(=O)N1CCn2c(c(-c3cn[nH]c3)c3ccc(Cl)c(Cl)c32)C1] |
| 325 | 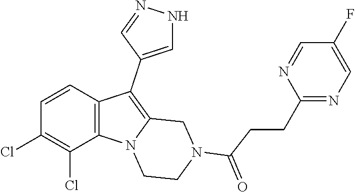 [SMILES: O=C(CCc1ncc(F)cn1)N1CCn2c(c(-c3cn[nH]c3)c3ccc(Cl)c(Cl)c32)C1] |
| 326 | 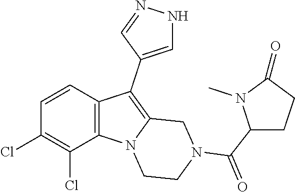 [SMILES: CN1C(=O)CCC1C(=O)N1CCn2c(c(-c3cn[nH]c3)c3ccc(Cl)c(Cl)c32)C1] |
| 327 | 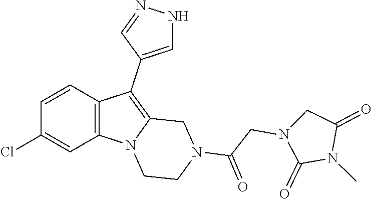 [SMILES: CN1C(=O)CN(CC(=O)N2CCn3c(c(-c4cn[nH]c4)c4ccc(Cl)cc43)C2)C1=O] |
| 328 | 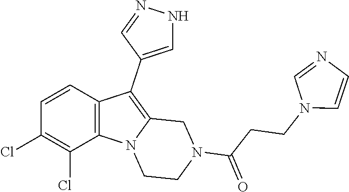 [SMILES: O=C(CCn1ccnc1)N1CCn2c(c(-c3cn[nH]c3)c3ccc(Cl)c(Cl)c32)C1] |
| 329 | 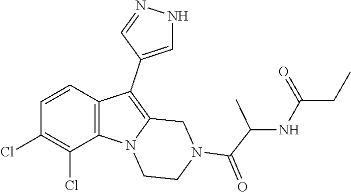 [SMILES: CCC(=O)NC(C)C(=O)N1CCn2c(c(-c3cn[nH]c3)c3ccc(Cl)c(Cl)c32)C1] |
| 330 | 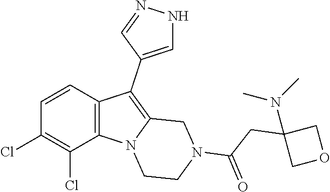 [SMILES: CN(C)C1(CC(=O)N2CCn3c(c(-c4cn[nH]c4)c4ccc(Cl)c(Cl)c43)C2)COC1] |
| 331 | 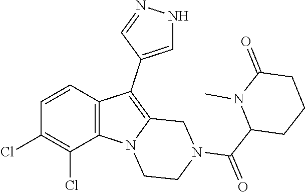 [SMILES: CN1C(=O)CCCC1C(=O)N1CCn2c(c(-c3cn[nH]c3)c3ccc(Cl)c(Cl)c32)C1] |
| 332 | 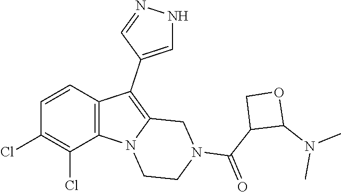 [SMILES: CN(C)C1OCC1C(=O)N1CCn2c(c(-c3cn[nH]c3)c3ccc(Cl)c(Cl)c32)C1] |
| 333 | 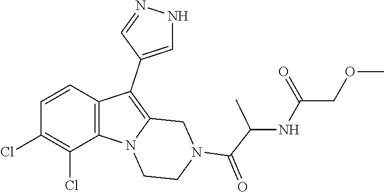 [SMILES: COCC(=O)NC(C)C(=O)N1CCn2c(c(-c3cn[nH]c3)c3ccc(Cl)c(Cl)c32)C1] |
| 334 | 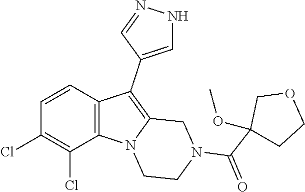 [SMILES: COC1(C(=O)N2CCn3c(c(-c4cn[nH]c4)c4ccc(Cl)c(Cl)c43)C2)CCOC1] |
| 335 | 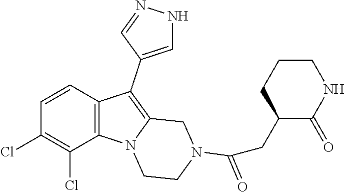 [SMILES: O=C1NCCC[C@@H]1CC(=O)N1CCn2c(c(-c3cn[nH]c3)c3ccc(Cl)c(Cl)c32)C1] |
| 336 | 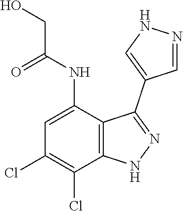 [SMILES: O=C(CO)Nc1cc(Cl)c(Cl)c2[nH]nc(-c3cn[nH]c3)c12] |
| 337 | 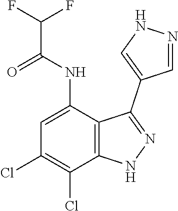 [SMILES: O=C(Nc1cc(Cl)c(Cl)c2[nH]nc(-c3cn[nH]c3)c12)C(F)F] |
| 338 | 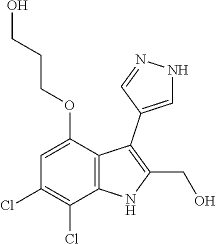 [SMILES: OCCCOc1cc(Cl)c(Cl)c2[nH]c(CO)c(-c3cn[nH]c3)c12] |
| 339 | 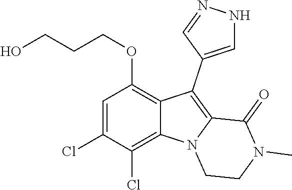 [SMILES: CN1CCn2c(c(-c3cn[nH]c3)c3c(OCCCO)cc(Cl)c(Cl)c32)C1=O] |
| 340 | 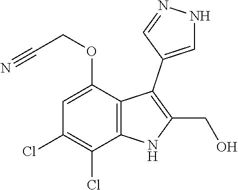 [SMILES: N#CCOc1cc(Cl)c(Cl)c2[nH]c(CO)c(-c3cn[nH]c3)c12] |
| 341 | 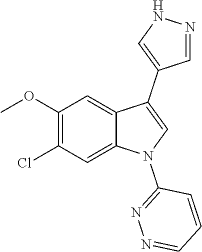 [SMILES: COc1cc2c(-c3cn[nH]c3)cn(-c3cccnn3)c2cc1Cl] |
| 342 | 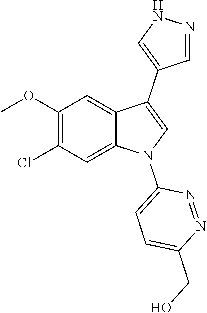 [SMILES: COc1cc2c(-c3cn[nH]c3)cn(-c3ccc(CO)nn3)c2cc1Cl] |
| 343 | 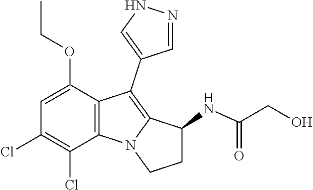 [SMILES: CCOc1cc(Cl)c(Cl)c2c1c(-c1cn[nH]c1)c1n2CC[C@@H]1NC(=O)CO] |
| 344 | 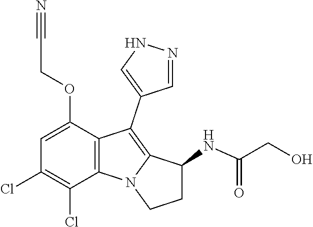 [SMILES: N#CCOc1cc(Cl)c(Cl)c2c1c(-c1cn[nH]c1)c1n2CC[C@@H]1NC(=O)CO] |
| 345 | 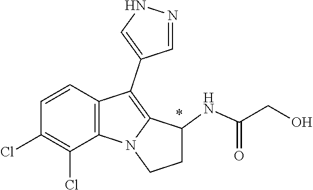 [SMILES: O=C(CO)NC1CCn2c1c(-c1cn[nH]c1)c1ccc(Cl)c(Cl)c12] Isomer 1 |
| 346 | 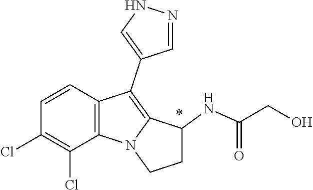 [SMILES: O=C(CO)NC1CCn2c1c(-c1cn[nH]c1)c1ccc(Cl)c(Cl)c12] Isomer 2 |
| 347 | 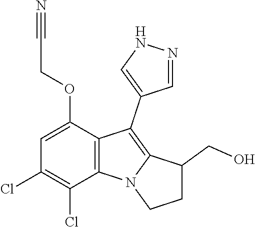 [SMILES: N#CCOc1cc(Cl)c(Cl)c2c1c(-c1cn[nH]c1)c1n2CCC1CO] |
| 348 | 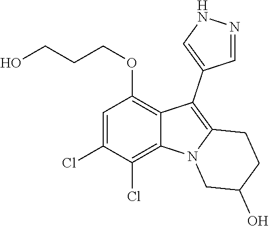 [SMILES: OCCCOc1cc(Cl)c(Cl)c2c1c(-c1cn[nH]c1)c1n2CC(O)CC1] |

### Assay

Expression and Purification of Recombinant cGAS Protein: Insert cDNA encoding full-length of human cGAS into a modified bacterial expression vector containing an in-frame His6-tag. Induce the E. coli strain BL21(DE3) harboring the plasmid with 1 mM IPTG (Isopropyl β-D-1-thiogalactopyranoside) at 18° C. overnight. Purify the His6-tagged cGAS protein over HISpur Ni-NTA resin in the presence of benzonase and RNase followed by Superdex 200 size exclusion chromatography with storage buffer (50 mM Tris-HCl pH 7.5, 300 mM NaCl, 1 mM DTT, 10% glycerol).

In vitro inhibition assay of cGAS activity: Add a 5 μL mixture containing 10 mM Tris-Cl pH 7.5, 50 mM NaCl, 5 mM MgCl₂, 1 mM DTT, 0.01% Triton X-100, 60 nM 75-bp dsDNA, 501.1M ATP, 50 μM GTP, 10 nM of recombinant human cGAS, and serial dilutions of a test compound in DMSO to a 384-well plate and incubate at room temperature for 3 hours. Quench the reaction by the addition of 100 μL of stop solution containing 0.1% formic acid and 0.1 μM internal standard (3′3′-difluoro cGAMP). Measure the amount of cGAMP produced by RapidFire 365 mass spectrometry and evaluate inhibitory effect of a compound by plotting percent specific inhibition of cGAMP production against log concentration of the test compound. Calculate IC₅₀ values using a 4-parameter non-linear logistic equation (y=(S0+((SInf−S0)/(1+((qAC50/x)\hat{ }nHill))))).

Cellular assay to measure cGAS activity: Use THP1 cell line from ATCC to determine inhibition of cGAS activity by test compounds in human cells. Plate cells on 96-well plates at 0.2×10⁶/well and differentiate with phorbol myristate acetate (PMA) at 37° C./5% CO₂. After 24 hours, replace PMA containing media with media lacking PMA and further incubate overnight. Stimulate cells were then by RDG-Adenovirus-GFP (Vector Biolabs #1768) at 1000 MOI for 4 hours prior to the addition of test compounds. After overnight incubation, transfer 10 μL of the media from each well to a new plate for IFNb analysis as a marker for cGAS activity. Measure quantification of IFNb with an IFNb AlphaLISA Detection kit (Perkin Elmer AL3133F) by mixing the acceptor beads with the cell supernatant followed by the addition of the biotinylated antibody and donor beads. Measure the relative fluorescence signal produced from the interaction of the donor and acceptor beads by a PHERAStar AlphaLISA protocol and evaluate the inhibitory effect of a compound by plotting specific inhibition of IFNb production against log concentration of the test compound. Calculate IC₅₀ values using a 4-parameter non-linear logistic equation (y=(S0+((SInf−S0)/(1+((qAC50/x)\hat{ }nHill))))).

cGAS inhibitory activity data for the Examples is summarized in Tables 7, 8, and 9 below.

TABLE 7
In vitro inhibition assay for Compounds 1-94

| Example | IC₅₀ (μM) |
|---|---|
| 1 | 0.0863 |
| 2 | 0.0557 |
| 3 | 0.0614 |
| 4 | 0.0641 |
| 5 | 0.0246 |
| 6 | 0.0578 |
| 7 | 0.0436 |
| 8 | 0.0531 |
| 10 | 0.0629 |
| 11 | 0.0423 |
| 12 | 0.0649 |
| 13 | 0.0117 |
| 14 | 0.0309 |
| 15 | 0.289 |
| 16 | 0.0674 |
| 17 | 0.0427 |
| 18 | 0.152 |
| 19 | 0.0663 |
| 20 | 0.155 |
| 21 | 0.0243 |
| 22 | 0.0225 |
| 23 | 0.0887 |
| 24 | 0.0834 |
| 25 | 0.0412 |
| 26 | 0.0229 |
| 27 | 0.158 |
| 28 | 0.064 |
| 29 | 0.0447 |
| 30 | 0.034 |
| 31 | 0.0462 |
| 32 | 0.0411 |
| 33 | 0.0825 |
| 34 | 0.0691 |
| 35 | 0.0737 |
| 36 | 0.0535 |
| 37 | 0.09 |
| 38 | 0.0404 |
| 39 | 0.0392 |
| 40 | 0.0525 |
| 41 | 0.0328 |
| 42 | 0.0273 |
| 43 | 0.0912 |
| 44 | 0.0534 |
| 45 | 0.0834 |
| 46 | 0.0270 |
| 47 | 0.0333 |
| 48 | 0.0443 |
| 49 | 0.0839 |
| 50 | 0.04 |
| 51 | 0.0427 |
| 52 | 0.0975 |
| 53 | 0.0268 |
| 54 | 0.0381 |
| 55 | 0.0946 |
| 56 | 0.0826 |
| 57 | 0.0352 |
| 58 | 0.0158 |
| 59 | 0.0242 |
| 60 | 0.0855 |
| 61 | 0.0326 |
| 62 | 0.0151 |
| 63 | 0.0273 |
| 64 | 0.0486 |
| 65 | 0.0158 |
| 66 | 0.0349 |
| 67 | 0.0371 |
| 68 | 0.0965 |
| 69 | 0.0449 |
| 70 | 0.0173 |
| 71 | 0.0463 |
| 72 | 0.0772 |
| 73 | 0.062 |
| 74 | 0.0847 |
| 75 | 0.045 |
| 76 | 0.0452 |
| 77 | 0.0157 |
| 78 | 0.0427 |
| 79 | 0.0753 |
| 80 | 0.0818 |
| 81 | 0.0223 |
| 82 | 0.046 |
| 83 | 0.0754 |
| 84 | 0.0815 |
| 85 | 0.0257 |
| 86 | 0.0485 |
| 87 | 0.029 |
| 88 | 0.0219 |
| 89 | 0.0112 |
| 90 | 0.0587 |
| 91 | 0.0405 |
| 92 | 0.0287 |
| 93 | 0.18 |
| 94 | 0.0224 |

TABLE 8
In vitro inhibition assay for Compounds 95-348

| Example | IC50 (μM) |
|---|---|
| 95 | 0.0159 |
| 96 | 0.0449 |
| 99 | 0.0593 |
| 100 | 0.0356 |
| 101 | 0.0705 |
| 102 | 0.0166 |
| 103 | 0.051 |
| 104 | 0.0872 |
| 105 | 0.0712 |
| 106 | 0.0561 |
| 107 | 0.0196 |
| 108 | 0.0621 |
| 109 | 0.0698 |
| 110 | 0.0561 |
| 111 | 0.0468 |
| 112 | 0.0361 |
| 113 | 0.018 |
| 114 | 0.0244 |
| 115 | 0.0288 |
| 116 | 0.0217 |
| 117 | 0.0153 |
| 118 | 0.0165 |
| 119 | 0.0186 |
| 120 | 0.0154 |
| 121 | 0.0652 |
| 122 | 0.0137 |
| 123 | 0.00491 |
| 124 | 0.0882 |
| 125 | 0.0103 |
| 126 | 0.0358 |
| 127 | 0.029 |
| 128 | 0.0691 |
| 129 | 0.0552 |
| 130 | 0.0711 |
| 131 | 0.0131 |
| 132 | 0.0202 |
| 133 | 0.0112 |
| 134 | 0.0282 |
| 135 | 0.0486 |
| 136 | 0.0764 |
| 137 | 0.0165 |
| 138 | 0.0462 |
| 139 | 0.077 |
| 140 | 0.0481 |
| 141 | 0.0275 |
| 142 | 0.0127 |
| 143 | 0.0419 |
| 144 | 0.00433 |
| 145 | 0.0804 |
| 146 | 0.0918 |
| 147 | 0.0474 |
| 148 | 0.0520 |
| 149 | 0.0442 |
| 150 | 0.0303 |
| 151 | 0.0349 |
| 152 | 0.0142 |
| 153 | 0.00511 |
| 154 | 0.026 |
| 155 | 0.0273 |
| 156 | 0.0405 |
| 157 | 0.0281 |
| 158 | 0.0507 |
| 159 | 0.019 |
| 160 | 0.0787 |
| 161 | 0.0616 |
| 162 | 0.0152 |
| 163 | 0.076 |
| 164 | 0.0543 |
| 165 | 0.0894 |
| 166 | 0.0931 |
| 167 | 0.0672 |
| 168 | 0.0463 |
| 169 | 0.0138 |
| 170 | 0.00567 |
| 171 | 0.00356 |
| 172 | 0.0341 |
| 173 | 0.0145 |
| 175 | 0.0585 |
| 176 | 0.0864 |
| 177 | 0.0245 |
| 178 | 0.0413 |
| 179 | 0.0585 |
| 180 | 0.018 |
| 181 | 0.0165 |
| 182 | 0.0416 |
| 183 | 0.0118 |
| 184 | 0.0385 |
| 185 | 0.077 |
| 186 | 0.0127 |
| 187 | 0.0104 |
| 188 | 0.0541 |
| 189 | 0.0272 |
| 190 | 0.0361 |
| 191 | 0.0763 |
| 192 | 0.0379 |
| 193 | 0.00322 |
| 194 | 0.0184 |
| 195 | 0.0298 |
| 196 | 0.064 |
| 197 | 0.00893 |
| 198 | 0.011 |
| 199 | 0.0519 |
| 200 | 0.0282 |
| 201 | 0.0777 |
| 202 | 0.0191 |
| 203 | 0.00677 |
| 204 | 0.0201 |
| 205 | 0.0287 |
| 206 | 0.0421 |
| 207 | 0.0420 |
| 208 | 0.0416 |
| 209 | 0.0158 |
| 210 | 0.0365 |
| 211 | 0.0342 |
| 212 | 0.0125 |
| 213 | 0.0538 |
| 214 | 0.0686 |
| 215 | 0.0864 |
| 216 | 0.0598 |
| 217 | 0.0794 |
| 218 | 0.0679 |
| 219 | 0.0348 |
| 220 | 0.0301 |
| 221 | 0.0238 |
| 222 | 0.0927 |
| 223 | 0.0812 |
| 224 | 0.0704 |
| 225 | 0.0121 |
| 226 | 0.0563 |
| 227 | 0.0408 |
| 228 | 0.0493 |
| 229 | 0.0965 |
| 230 | 0.0693 |
| 231 | 0.0724 |
| 232 | 0.00376 |
| 233 | 0.0806 |
| 234 | 0.0249 |
| 235 | 0.019 |
| 236 | 0.0331 |
| 237 | 0.0150 |
| 238 | 0.046 |
| 239 | 0.0613 |
| 240 | 0.0642 |
| 241 | 0.0625 |
| 242 | 0.464 |
| 243 | 0.0472 |
| 244 | 0.0099 |
| 245 | 0.0122 |
| 246 | 0.0313 |
| 247 | 0.0146 |
| 248 | 0.0396 |
| 249 | 0.0306 |
| 250 | 0.0052 |
| 251 | 0.0265 |
| 252 | 0.0330 |
| 253 | 0.014 |
| 254 | 0.030 |
| 255 | 0.0326 |
| 256 | 0.0386 |
| 257 | 0.0235 |
| 258 | 0.0757 |
| 259 | 0.0373 |
| 260 | 0.0115 |
| 261 | 0.0452 |
| 262 | 0.0772 |
| 263 | 0.0173 |
| 264 | 0.0462 |
| 265 | 0.0511 |
| 266 | 0.0401 |
| 267 | 0.0399 |
| 268 | 0.0385 |
| 269 | 0.0306 |
| 270 | 0.0207 |
| 271 | 0.184 |
| 272 | 0.0202 |
| 273 | 0.0206 |
| 274 | 0.023 |
| 275 | 0.0284 |
| 276 | 0.0405 |
| 277 | 0.0445 |
| 278 | 0.0391 |
| 279 | 0.0072 |
| 280 | 0.0087 |
| 281 | 0.0089 |
| 282 | 0.00896 |
| 283 | 0.0106 |
| 284 | 0.0124 |
| 285 | 0.0126 |
| 286 | 0.0131 |
| 287 | 0.0203 |
| 288 | 0.0242 |
| 289 | 0.0303 |
| 290 | 0.0321 |
| 291 | 0.0325 |
| 292 | 0.0365 |
| 293 | 0.0447 |
| 294 | 0.0479 |
| 295 | 0.0561 |
| 296 | 0.061 |
| 297 | 0.0251 |
| 298 | 0.0234 |
| 299 | 0.0257 |
| 300 | 0.0288 |
| 301 | 0.0291 |
| 302 | 0.0323 |
| 303 | 0.0349 |
| 304 | 0.0351 |
| 305 | 0.0378 |
| 306 | 0.0379 |
| 307 | 0.0398 |
| 308 | 0.0423 |
| 309 | 0.0427 |
| 310 | 0.0428 |
| 311 | 0.0430 |
| 312 | 0.0468 |
| 313 | 0.0489 |
| 314 | 0.0491 |
| 315 | 0.0503 |
| 316 | 0.0509 |
| 317 | 0.0516 |
| 318 | 0.0524 |
| 319 | 0.0534 |
| 320 | 0.0550 |
| 321 | 0.0562 |
| 322 | 0.060 |
| 323 | 0.0613 |
| 324 | 0.0626 |
| 325 | 0.0626 |
| 326 | 0.0627 |
| 327 | 0.0635 |
| 328 | 0.0636 |
| 329 | 0.0682 |
| 330 | 0.0801 |
| 331 | 0.0830 |
| 332 | 0.0852 |
| 333 | 0.0918 |
| 334 | 0.0985 |
| 335 | 0.0162 |
| 336 | 0.0385 |
| 337 | 0.0124 |
| 338 | 0.00474 |
| 339 | 0.00789 |
| 340 | 0.0102 |
| 341 | 0.0276 |
| 342 | 0.0261 |
| 343 | 0.00614 |
| 344 | 0.00338 |
| 345 | 0.0628 |
| 346 | 0.00769 |
| 347 | 0.0122 |
| 348 | 0.00695 |

TABLE 9
Cellular assay for Compounds 95-348

| Example | IC50 (μM) |
|---|---|
| 95 | 2.06 |
| 96 | 2.43 |
| 100 | >10.0 |
| 102 | 1.28 |
| 103 | >10.0 |
| 107 | 5.56 |
| 111 | >10.0 |
| 112 | 1.56 |
| 113 | 3.46 |
| 114 | 1.53 |
| 115 | 1.12 |
| 116 | 5.23 |
| 117 | 2.49 |
| 118 | 3.61 |
| 119 | 3.66 |
| 120 | 2.27 |
| 121 | 2.99 |
| 122 | 0.880 |
| 123 | 0.547 |
| 124 | >10.0 |
| 125 | 0.426 |
| 126 | 1.45 |
| 127 | 0.216 |
| 128 | >10.0 |
| 129 | 0.895 |
| 130 | 0.815 |
| 131 | 0.163 |
| 132 | 0.317 |
| 133 | 0.498 |
| 134 | 1.59 |
| 135 | >10.0 |
| 136 | 3.42 |
| 137 | 1.38 |
| 138 | 4.99 |
| 139 | 5.13 |
| 140 | 7.11 |
| 141 | 3.3 |
| 142 | 3.14 |
| 143 | >10.0 |
| 144 | 1.47 |
| 145 | >10.0 |
| 146 | >10.0 |
| 147 | 4.63 |
| 148 | 1.54 |
| 149 | 6.05 |
| 150 | 1.92 |
| 151 | >10.0 |
| 152 | 1.40 |
| 153 | 0.807 |
| 154 | 2.41 |
| 155 | 7.68 |
| 156 | 0.464 |
| 157 | 0.331 |
| 158 | 0.363 |
| 159 | >10.0 |
| 160 | 0.536 |
| 161 | 1.01 |
| 162 | 0.761 |
| 163 | 2.09 |
| 164 | 1.34 |
| 165 | 1.32 |
| 166 | 2.83 |
| 167 | 1.86 |
| 168 | 1.00 |
| 169 | 0.656 |
| 170 | 0.129 |
| 171 | 0.0689 |
| 172 | 0.651 |
| 173 | 0.466 |
| 177 | 1.13 |
| 178 | 3.62 |
| 180 | 1.85 |
| 181 | >10.0 |
| 182 | >10.0 |
| 183 | 0.875 |
| 184 | 1.13 |
| 185 | 3.44 |
| 186 | 0.909 |
| 187 | 0.326 |
| 188 | 2.45 |
| 189 | 1.89 |
| 190 | 2.19 |
| 191 | 8.53 |
| 192 | 1.29 |
| 193 | 0.143 |
| 194 | 1.29 |
| 195 | 1.23 |
| 196 | >10.0 |
| 197 | 1.06 |
| 198 | 0.395 |
| 199 | 2.66 |
| 200 | 0.292 |
| 201 | 4.23 |
| 202 | 2.68 |
| 203 | 0.374 |
| 204 | 0.854 |
| 205 | 1.32 |
| 206 | 1.28 |
| 207 | 1.63 |
| 208 | 2.54 |
| 209 | 0.351 |
| 210 | 2.17 |
| 211 | 2.28 |
| 212 | >10.0 |
| 213 | 4.1 |
| 214 | 0.885 |
| 215 | 2.59 |
| 216 | 4.17 |
| 217 | 2.12 |
| 218 | 3.27 |
| 219 | 0.350 |
| 220 | 0.556 |
| 221 | >10.0 |
| 222 | >10.0 |
| 223 | 3.2 |
| 224 | 2.73 |
| 225 | 0.230 |
| 226 | 2.27 |
| 227 | 1.60 |
| 228 | 1.46 |
| 229 | 2.10 |
| 230 | 2.02 |
| 231 | 1.01 |
| 232 | 0.668 |
| 233 | 1.13 |
| 234 | 1.72 |
| 235 | 2.05 |
| 236 | >10.0 |
| 237 | 1.74 |
| 238 | >10.0 |
| 239 | >10.0 |
| 240 | >10.0 |
| 241 | >10.0 |
| 242 | >10.0 |
| 243 | 9.35 |
| 244 | 0.531 |
| 245 | 0.872 |
| 246 | 9.23 |
| 247 | 2.73 |
| 248 | >10.0 |
| 249 | 2.32 |
| 250 | 0.376 |
| 251 | 1.36 |
| 252 | 0.726 |
| 253 | 0.509 |
| 254 | 0.938 |
| 255 | 2.09 |
| 256 | 4.8 |
| 257 | >10.0 |
| 258 | >10.0 |
| 259 | 0.765 |
| 260 | 4.05 |
| 261 | 2.34 |
| 262 | 3.62 |
| 263 | 0.509 |
| 264 | >10.0 |
| 265 | >10.0 |
| 266 | 9.93 |
| 267 | 6.27 |
| 268 | 5.87 |
| 269 | >10.0 |
| 270 | 0.635 |
| 271 | 6.75 |
| 272 | 0.412 |
| 273 | 0.56 |
| 274 | 3.55 |
| 275 | 4.01 |
| 276 | >10.0 |
| 277 | 1.58 |
| 278 | 2.19 |
| 279 | 0.740 |
| 280 | 0.872 |
| 281 | 1.00 |
| 282 | 2.99 |
| 283 | 4.27 |
| 284 | 9.75 |
| 285 | 1.19 |
| 286 | 0.455 |
| 287 | 5.11 |
| 288 | 0.560 |
| 289 | 3.73 |
| 290 | 6.97 |
| 291 | 4.91 |
| 292 | 4.08 |
| 293 | >10.0 |
| 294 | 0.366 |
| 295 | 2.14 |
| 296 | 4.78 |
| 297 | >10.0 |
| 298 | >10.0 |
| 299 | >10.0 |
| 300 | >10.0 |
| 301 | >10.0 |
| 302 | 6.95 |
| 303 | >10.0 |
| 304 | 8.47 |
| 305 | >10.0 |
| 306 | >10.0 |
| 307 | >10.0 |
| 308 | 7.9 |
| 309 | >10.0 |
| 310 | >10.0 |
| 335 | 8.94 |
| 336 | 0.492 |
| 337 | 0.281 |
| 338 | 0.198 |
| 339 | 0.426 |
| 340 | 0.516 |
| 341 | 0.80 |
| 342 | 0.964 |
| 343 | 0.565 |
| 344 | 0.902 |
| 345 | 3.97 |
| 346 | 0.914 |
| 347 | 0.865 |
| 348 | 0.365 |

The data above shows that the compounds of the invention are effective cGAS inhibitors. Other compounds of the disclosure not specifically exemplified can also be similarly tested and verified to be effective cGAS inhibitors.

1. A compound of formula: 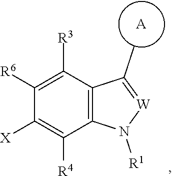
wherein:
ring A is 5-membered heteroaryl containing 1, 2, or 3 nitrogen atoms, wherein the heteroaryl is optionally substituted with 1, 2, or 3 C₁₋₃ alkyl;
X is halo;
W is CR² or N;
R² is H, —C₁₋₃ alkyl, —CR⁵R⁷R⁸, —C(O)—R^{d}, —CH═N—R^{v}, or R^{w};
R¹ is H, —C₁₋₃ alkyl, —R^{a}—C(O)OH, —R^{a}—NH—C(O)CH₃, —R^{a}—NHS(O)₂CH₃, or 5- or 6-membered heteroaryl containing 1, 2, or 3 nitrogen atoms optionally substituted with one or more R^{m};
R⁵ is —R^{a}—OH, —OR^{b}, —N(R^{b})—C(O)—R^{a}—H, —N(R^{b})—C(O)—(R^{a})ₜ—N(R^{b})₂, —N(R^{b})—C(O)—(R^{a})ₜ—N(R^{b})—Rⁱ, —N(R^{b})—C(O)—(R^{a})ₜ—N(R^{b})—(R^{a})ₜ—C(O)—R^{b}, —N(R^{b})—C(O)—R^{d}, —N(R^{b})—R^{a}—R^{d}, —N(R^{b})—(C(O))ₜ—(R^{a})ₜ—Rⁱ, —N(R^{b})—S(O)₂—R^{a}—H, —C(O)—N(R^{b})—R^{a}—R¹, —N(R^{b})—C(S)—R^{a}—H, or —R^{a}—C(O)OH;
or R¹ and R⁵, together with the atoms to which they are immediately attached, form a 5-, 6-, or 7-membered heterocyclic ring containing 1 or 2 nitrogen atoms, wherein the heterocyclic ring is optionally substituted with one or more R^{m};
R³ is H, —YR^{b}, —YR^{c}, —YR^{d}, —(NR^{b})ₙ—R^{a}—H, —Y—R^{a}—CN, —Y—C(O)R^{a}—H, —Y—R^{a}—C(O)N(R^{b})₂, —Y—C(S)R^{a}—H, —Y—C(O)R^{a}—F, —Y—C(O)—R^{a}—CN, or —Y—R^{a}—CH═CH—R^{a}—OH, wherein when R³ is H, then R² is other than H, —C₁₋₃ alkyl, —OH, or —C₁₋₃ alkoxy;
R⁴ is H, halo, or —CN, wherein when R⁴ is H or F, then either (1) R¹ is 5-member heteroaryl containing three nitrogen that is optionally substituted with one of —R^{a}—H, —R^{a}—NH₂, and —R^{a}—C(O)NH₂, or (2) R₃ is —NH—C(O)—CF₂H or —NH—C(O)—CH₂F;
R⁶ is H, halo, or —O—R^{a}—H;
R⁷ and R⁸ are each R^{b}, or R⁷ and R⁸ collectively forms an oxo;
each occurrence of R^{a} is independently —C₁₋₃ alkylene- optionally substituted with one or more substituents selected from the group consisting of halo, —OH, —C₁₋₃ alkyl, —C₁₋₃ alkylene-OH, and —C₁₋₃ alkoxy;
each occurrence of R^{b} is independently —R^{a}—H or —H;
R^{c} is —C₃₋₆ cycloalkyl optionally substituted with one or more —OH, —R^{a}—H, or halo;
R^{d} is 4-8 membered heterocyclyl optionally substituted with oxo, —OH, —C₁₋₃ alkylcarbonyl, or —COOH;
each occurrence of L is selected from the group consisting of —C(O)—, —C(O)—N(R^{b})—, —C(O)—O—, —N(R^{b})—C(O)—, —O—C(O)—, —S(O)₂—, —N(R^{b})—S(O)₂—, and —N(R^{b})C(S)—;
each occurrence of Y is —O— or —N(R^{b})—;
each occurrence of R^{f} is selected from the group consisting of —R^{b}, —Rⁱ, —OR^{b}, —N(R^{b})₂, —(Y)ₜ—(R^{a})ₜ—R^{v}, and -L-R^{b};
each occurrence of Rⁱ is acyl or a 5- to 6-membered heteroaryl containing 1, 2, or 3 heteroatoms independently selected from nitrogen, oxygen, and sulfur and optionally substituted with —C₁₋₃ alkyl;
each occurrence of R^{w} is a 5- or 6-membered heteroaryl, wherein the heteroaryl includes three nitrogen or a combination of two nitrogen with a chalcogen, and wherein the heteroaryl is optionally substituted with —R^{b}, —R^{a}—OH, —CF₃, —N(R^{b})₂, —NHC(O)R^{a}—OH, —SR^{b}, or —R^{c}, wherein when the heteroaryl is a 5-membered heteroaryl with three nitrogen, it is substituted with —SR^{b};
each occurrence of R^{m} is each independently selected from the group consisting of -(L)ₜ-(R^{a})ₜ-(L)ₜ-(R^{a})ᵥ—R^{f}, -(L)ₜ-(R^{a}—O)_{q}—R^{a}—R^{f}, and —Y—Rⁱ; or two of R^{m} collectively forms an oxo;
each occurrence of R^{v} is independently a 4-8 membered heterocycle or a 5-6 membered heteroaryl, each optionally substituted with one or more groups selected from C₁₋₃ alkyl, halo, oxo, acyl, —R^{a}—OR^{b}, —NR^{b}₂, —OR^{b}, —C(O)—R^{b}, —C(O)—OR^{b}, —C(O)—R^{d}, C₁₋₃ cycloalkyl, and —SR^{b};
each occurrence of n is independently 1, 2 or 3;
each occurrence oft is independently 0 or 1;
each occurrence of q is independently 1, 2, or 3; and
each occurrence of v is independently 0, 1, or 2,
or a pharmaceutically acceptable salt thereof.

2. (canceled)

3. The compound of claim 1, wherein the compound is of formula: 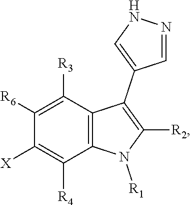
or a pharmaceutically acceptable salt thereof.

4. The compound of claim 3, wherein R² is an optionally substituted 5-membered heteroaryl including two nitrogen and a chalcogen selected from oxygen and sulfur, or a pharmaceutically acceptable salt thereof.

5. The compound of claim 3, wherein R² is H, —CH₂OH, —CH₂—NH—C(O)—CH₃, or —CH₂—NH—C(O)—CH₂—OH, or a pharmaceutically acceptable salt thereof.

6. (canceled)

7. The compound of claim 1, having formula: 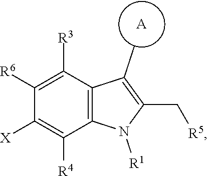
or a pharmaceutically acceptable salt thereof.

8. The compound of claim 7, wherein R⁵ is —OH, —CH₂OH, —C₁₋₃ alkoxy, —NHRⁱ, —NHC(O)Rⁱ, —NHC(O)NH₂, —NH—C(O)—R^{a}—H, —NH—C(S)—R^{a}—H, —C(O)NHCH₂Rⁱ, —NHC(O)CH₂Rⁱ, —NHC(O)CH₂N(R^{b})₂, —NHC(O)CH₂NHC(O)—R^{b}, or —CH₂C(O)OH, or a pharmaceutically acceptable salt thereof.

9. (canceled)

10. The compound of claim 1, wherein the compound is of formula: 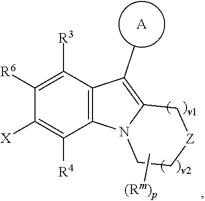
wherein:
Z is CR^{m} or N,
v1 and v2 are each 0, 1, or 2 and v1+v2 is 1, 2, or 3, and
p is 0, 1, 2, 3, or 4, and p is not more than v1+v2+1,
or a pharmaceutically acceptable salt thereof.

11. The compound of claim 1, wherein the compound is of a formula: 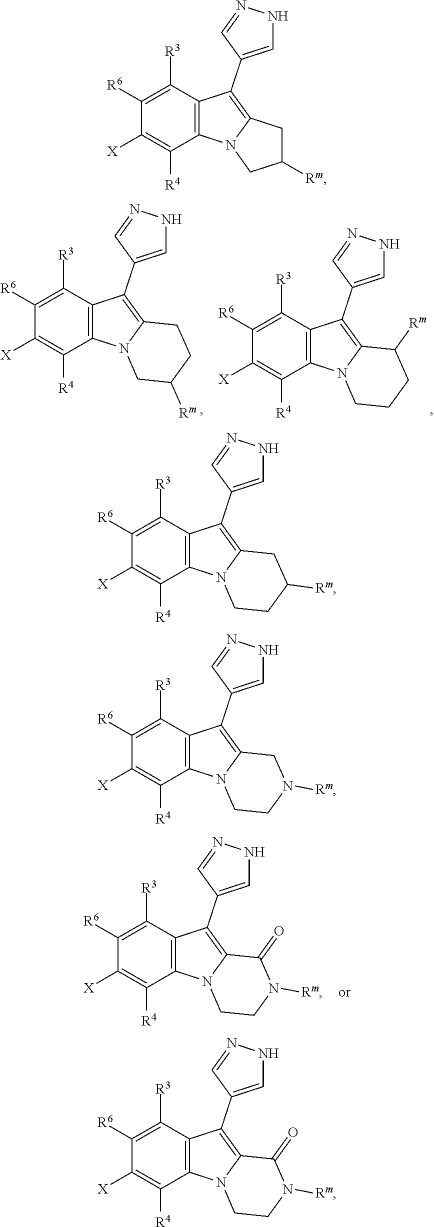
or a pharmaceutically acceptable salt thereof.

12. (canceled)

13. The compound of claim 1, wherein the compound is of formula: 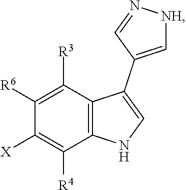
or a pharmaceutically acceptable salt thereof.

14. The compound of claim 1, wherein the compound is of formula: 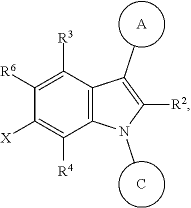
wherein ring C is 5-membered heteroaryl containing 1, 2, or 3 nitrogen and optionally substituted with one or more R^{m}, or a pharmaceutically acceptable salt thereof.

15. The compound of claim 1, wherein R³ is —YR^{b}, —YR^{c}, —YR^{d}, —(NR^{b})ₙ—R^{a}—H, —Y—R^{a}—CN, —Y—C(O)R^{a}—H, —Y—C(S)R^{a}—H, —Y—C(O)R^{a}—F, —Y—C(O)—R^{a}—CN, or —Y—R^{a}—CH═CH—R^{a}—OH, or a pharmaceutically acceptable salt thereof.

16. The compound of claim 1, wherein R³ is H, —NH—C(O)—CH₃, —NH—C(O)—CHF₂, —O—(CH₂)₃—OH, —O—CH₂—CN, or —NH—(CH₂)₃—OH, or a pharmaceutically acceptable salt thereof.

17. The compound of claim 1, wherein R³ is
—(NH)ₙCH₃,
—NH—C(O)—R^{a}—CN,
—NH—R^{a}—CN,
—O—R^{a}—CN,
—NH—C(O)—CF₃,
—NHC(S)CH₃,
—NH—R^{a}—C(O)NH₂,
—O—R^{a}—C(O)NH₂,
—NH—R^{a}—CH═CH—R^{a}—OH,
—NH—C(O)—R^{a}—H wherein R^{a} is —C₁₋₃ alkylene- optionally substituted with one or two —OH, one or two F, —C₁₋₃ alkylene-OH, or combinations thereof,
—NH—R^{a}—H wherein R^{a} is —C₁₋₃ alkylene- optionally substituted with one or two —OH, one or two F, —C₁₋₃ alkylene-OH, or combinations thereof,
—O—R^{a}—H wherein R^{a} is —C₁₋₃ alkylene- optionally substituted with one or two —OH, one or two F, —C₁₋₃ alkylene-OH, or combinations thereof,
—OR^{c} wherein R^{c} is —C₃₋₆ cycloalkyl substituted with one or more —OH, —C₁₋₃ alkylene-OH, or halo,
—NHR^{c} wherein R^{c} is —C₃₋₆ cycloalkyl substituted with one or more —OH, —C₁₋₃ alkylene-OH, or halo,
—NHR^{d}, or
—OR^{d},
or a pharmaceutically acceptable salt thereof.

18. (canceled)

19. The compound of claim 1, wherein R⁴ is Cl, or a pharmaceutically acceptable salt thereof.

20-24. (canceled)

25. The compound of claim 1, wherein R^{m} is each independently selected from H, —OH, —NH₂, —R^{a}—H, —R^{v}, —R^{a}—R^{v}, —C(O)R^{a}—H, —C(O)R^{v}, —C(O)—R^{a}—R^{v}, —R^{a}—NH₂, —C(O)NHCH₃, —NHC(O)—R^{a}—H, —NHC(O)—R^{a}—NHC(O)R^{b}, —NHC(O)R^{d}, —NHC(O)R^{a}—OR^{b}, —NHC(O)—(R^{a}—O)_{q}—R^{a}—NH₂, —NHC(O)—R^{a}—NH—C(O)—R^{a}—H, —NHC(O)—R^{a}—C(O)—NR^{b}₂, —NHC(O)—R^{a}—NH—C(O)—(R^{a}), —NH₂, —(R^{a}—O)_{q}—R^{a}—H, —R^{a}—OH, —R^{a}—O—R^{a}—H, —C(O)OH, —CH₂C(O)OH, —CH₂CONH₂, —C(O)R^{a}—H, —C(O)—R^{a}—OR^{b}, —C(O)—(R^{a}—O)—(R^{a}—O)—R^{b}, —C(O)—R^{a}—N(R^{b})—C(O)R^{a}—OR^{b}, —C(O)—(R^{a}—O)—R^{a}—R^{v}, —C(O)—R^{a}—N(R^{b})C(O)R^{a}—H, —C(O)—R^{a}—N(R^{b})C(O)R^{a}—OR^{b}, —C(O)—R^{a}—N(R^{b})C(O)R^{a}—N(R^{b})₂, —C(O)—R^{a}—O—R^{a}—C(O)N(R^{b})₂, —C(O)—CH(—C₁₋₃ alkoxy)-R^{a}—OR^{b}, —C(O)R^{a}—C(O)N(R^{b})₂, —C(O)—R^{a}—N(R^{b})C(O)—R^{a}—H, —S(O)₂NH₂, —S(O)₂CH₃, —NHS(O)₂CH₃, —NHS(O)₂R^{a}—R^{v}, —NHS(O)₂R^{a}—NH—C(O)—R^{a}—H, —NHS(O)₂R^{a}—C(O)—NH—R^{a}—H, and —NHS(O)₂R^{a}—O—R^{a}—H; or
two of R^{m} collectively forms an oxo;
or a pharmaceutically acceptable salt thereof.

26. The compound of claim 1, wherein R^{m} is each independently selected from the group consisting of: H, C₁₋₃ alkyl, —OH, —C₁₋₃ alkylene-OH, —C₁₋₃ alkylene-NH₂, C₁₋₃ alkoxy, —C(O)—OH, 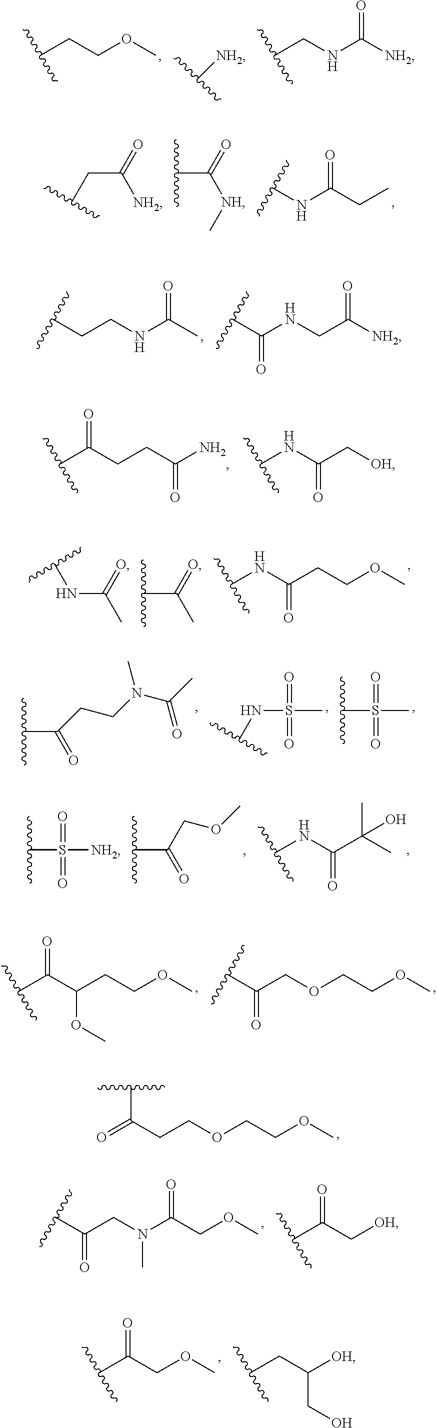 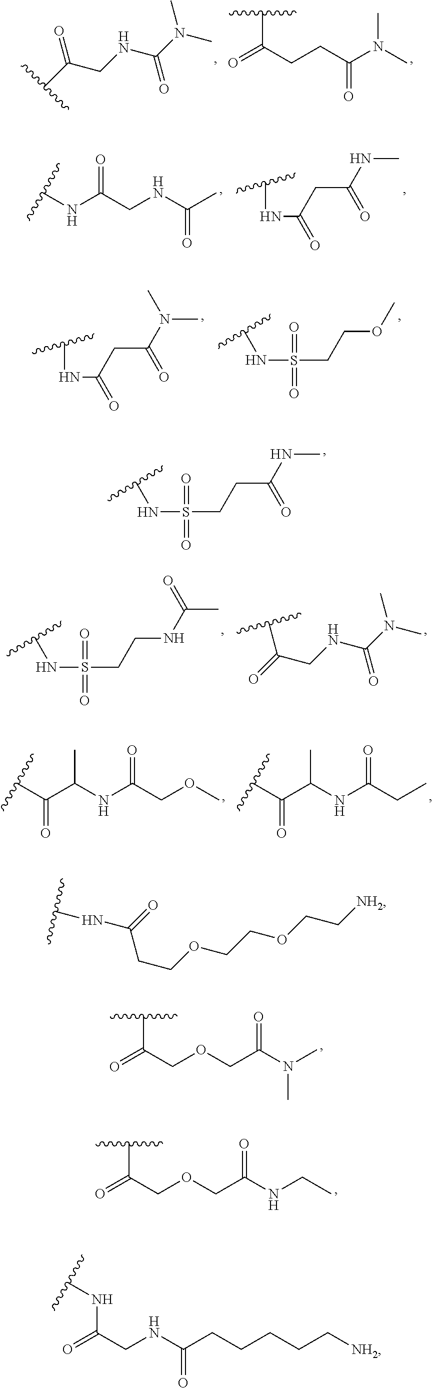 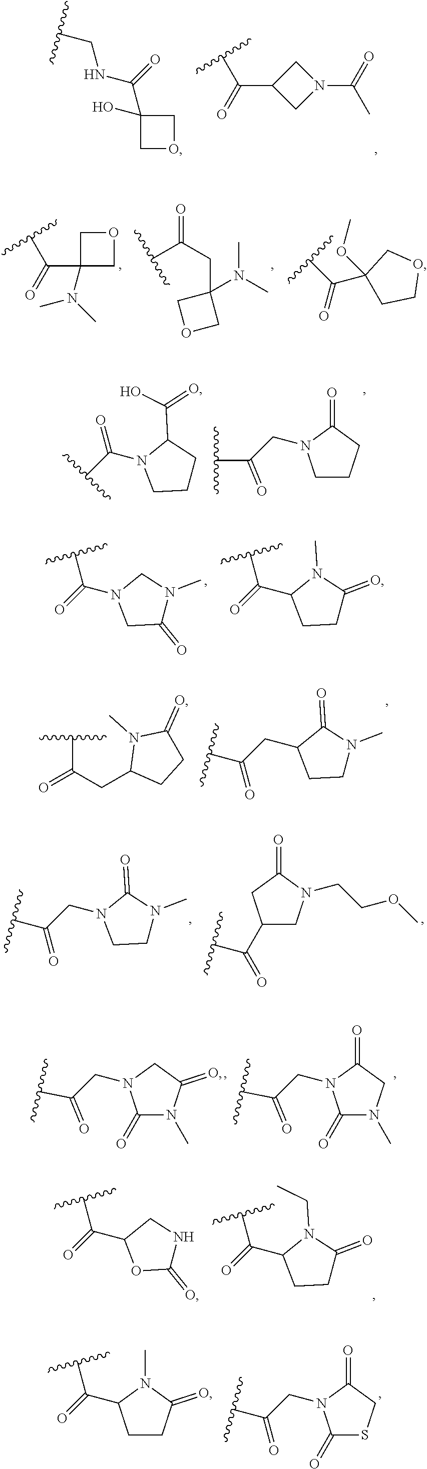 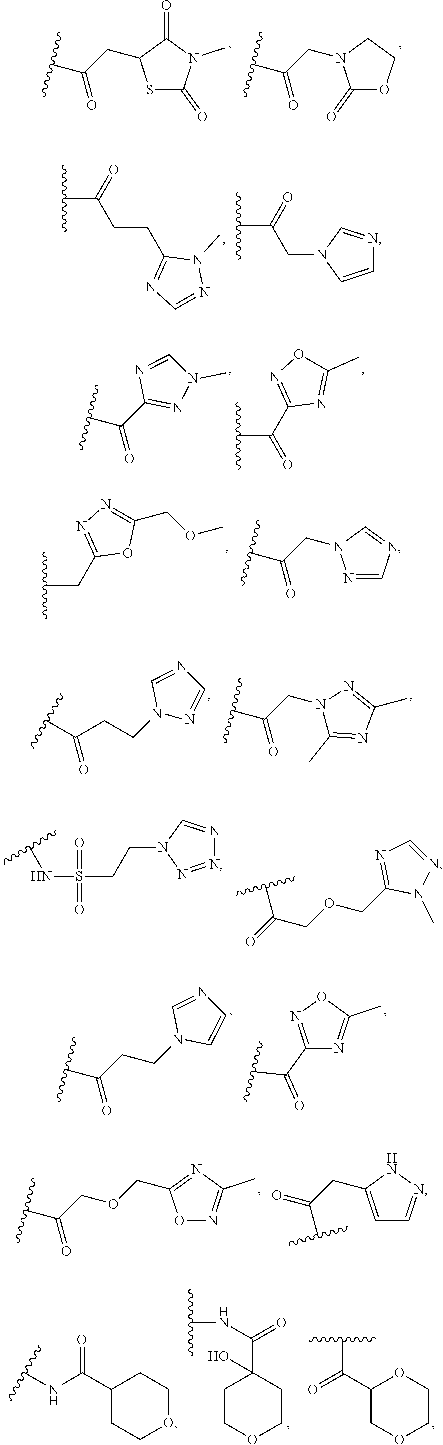 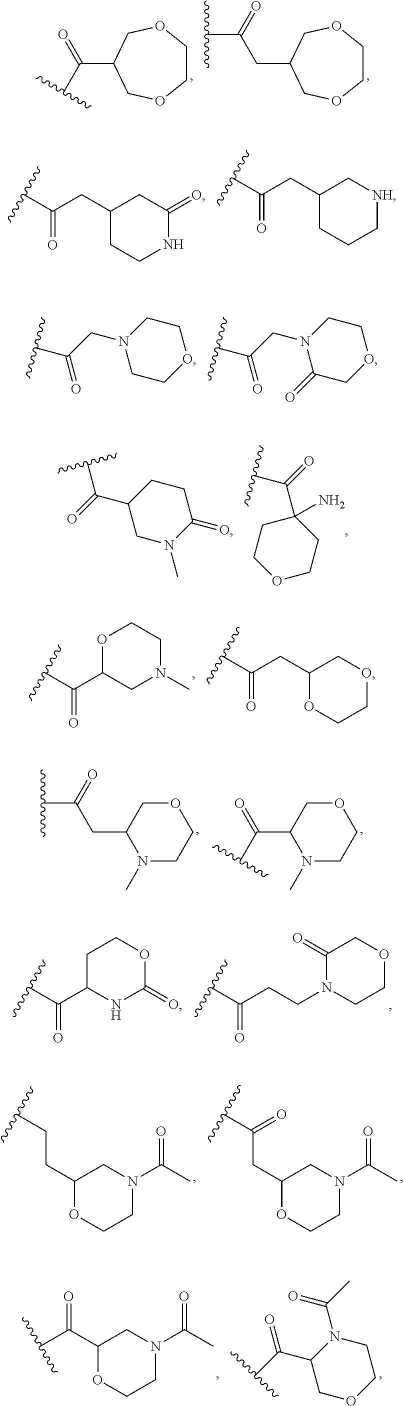 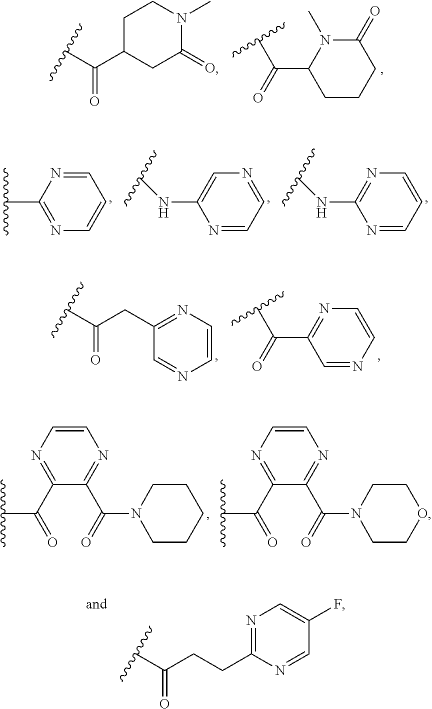
or two R^{m} collectively forms an oxo,
or a pharmaceutically acceptable salt thereof.

27. The compound of claim 1, wherein R¹ is H or CH₃, or a pharmaceutically acceptable salt thereof.

28. The compound of claim 1, wherein X is Cl, or a pharmaceutically acceptable salt thereof.

29. The compound of claim 1, wherein:
R⁴ is Cl;
X is Cl;
R⁶ is H;
W is CR²;
R² is H, —C₁₋₃ alkyl, —CR⁵R⁷R⁸, —C(O)—R^{d}, or R^{w};
R¹ is H, C₁₋₃ alkyl, —R^{a}—C(O)OH, —R^{a}—NH—C(O)CH₃, —R^{a}—NHS(O)₂CH₃, or 5-membered heteroaryl containing 1, 2, or 3 nitrogen atoms optionally substituted with one or more R^{m};
R⁵ is —R^{a}—OH, —OR^{b}, —N(R^{b})—C(O)—R^{a}—H, —N(R^{b})—C(O)—(R^{a})ₜ—N(R^{b})₂, —N(R^{b})—C(O)—(R^{a})ₜ—N(R^{b})—Rⁱ, —N(R^{b})—C(O)—(R^{a})ₜ—N(R^{b})—(R^{a})ₜ—C(O)—R^{b}, —N(R^{b})—C(O)—R^{d}, —N(R^{b})—R^{a}—R^{d}, —N(R^{b})—(C(O))ₜ—(R^{a})ₜ—Rⁱ, —C(O)—N(R^{b})—R^{a}—R¹, or —R^{a}—C(O)OH;
or R¹ and R⁵, together with the atoms to which they are immediately attached, form a 5-, 6-, or 7-membered heterocyclic ring containing 1 or 2 nitrogen atoms, wherein the heterocyclic ring is optionally substituted with one or more R^{m};
R³ is H, —YR^{b}, —YR^{c}, —YR^{d}, —(NR^{b})ₙ—R^{a}—H, —Y—R^{a}—CN, —Y—C(O)R^{a}—H, —Y—R^{a}—C(O)N(R^{b})₂, —Y—C(S)R^{a}—H, —Y—C(O)R^{a}—F, or —Y—C(O)—R^{a}—CN, wherein when R³ is H, then R² is other than H, —C₁₋₃ alkyl, —OH, or —C₁₋₃ alkoxy; and
ring A is 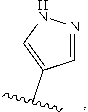 [SMILES: *c1cn[nH]c1]
or a pharmaceutically acceptable salt thereof.

30. The compound of claim 1, wherein:
R⁴ is Cl;
X is Cl;
R⁶ is H;
W is CR²;
R¹ is selected from the group consisting of: H, —C₁₋₃ alkyl, —R^{a}—C(O)OH, —R^{a}—NH—C(O)CH₃, —R^{a}—NHS(O)₂CH₃, 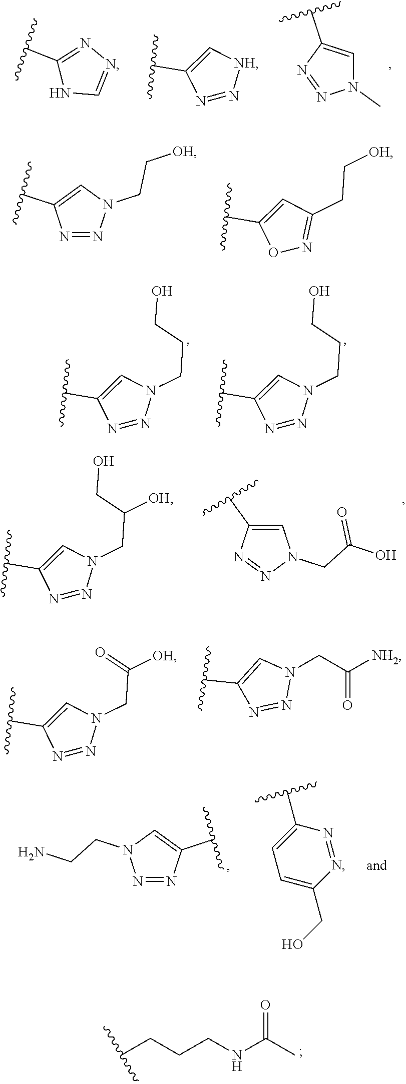
R² is selected from the group consisting of H, C₁₋₃ alkyl, —C₁₋₃ alkylene-OH, C₁₋₃ alkoxy, 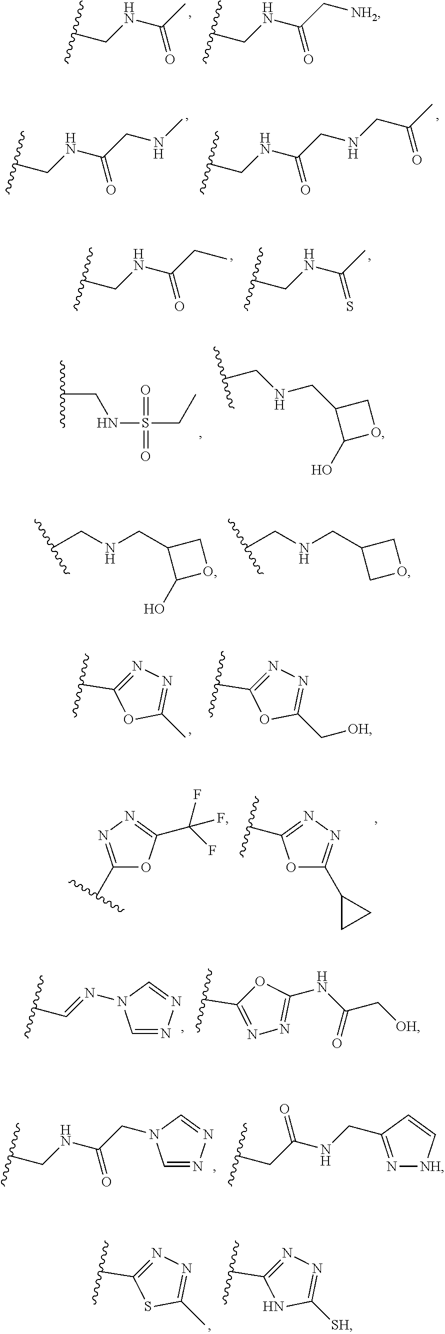 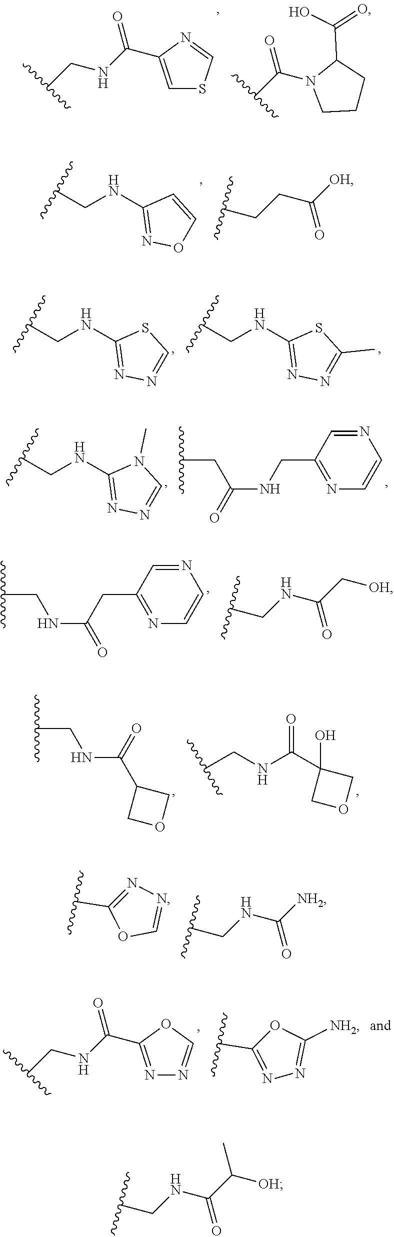
and
R³ is selected from the group consisting of H, C₁₋₃ alkoxy, 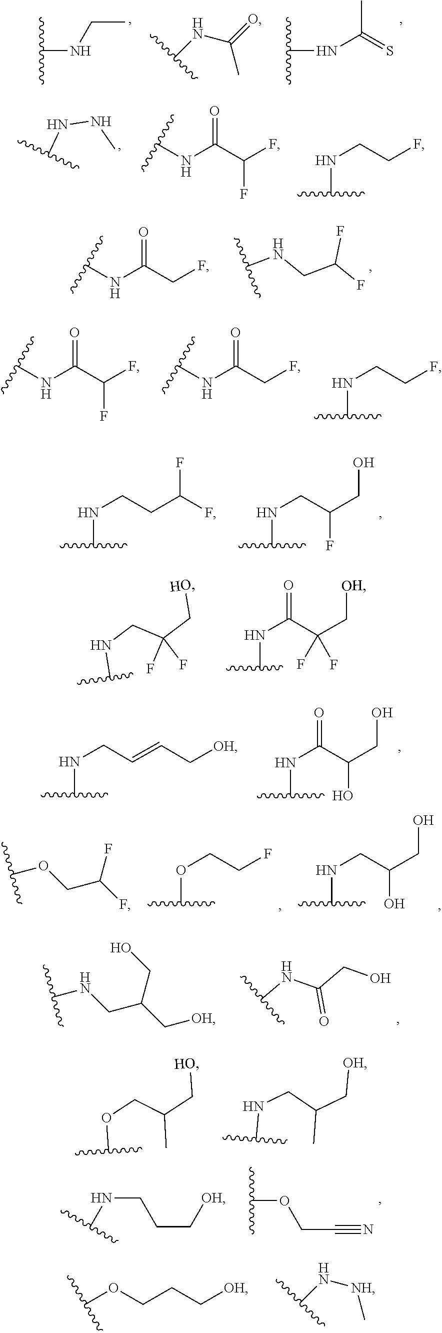 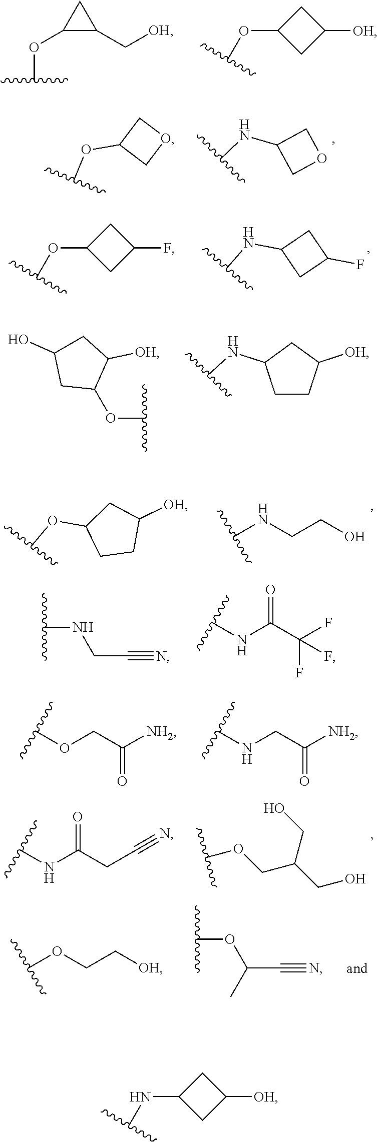
or a pharmaceutically acceptable salt thereof.

31. The compound of claim 1, having formula: 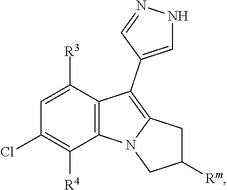
wherein
R³ is 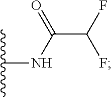 [SMILES: *NC(=O)C(F)F]
R⁴ is H or halo; and
R^{m} is selected from the group consisting of: —C₁₋₃ alkylene-OH, 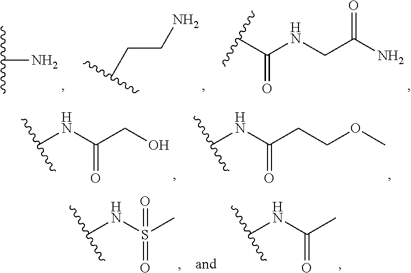
or a pharmaceutically acceptable salt thereof.

32. The compound of claim 1, having formula: 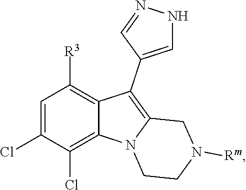
wherein:
R³ is selected from the group consisting of: 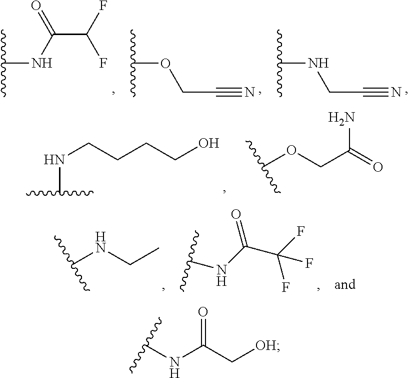
and
R^{m} is selected from the group consisting of: H, 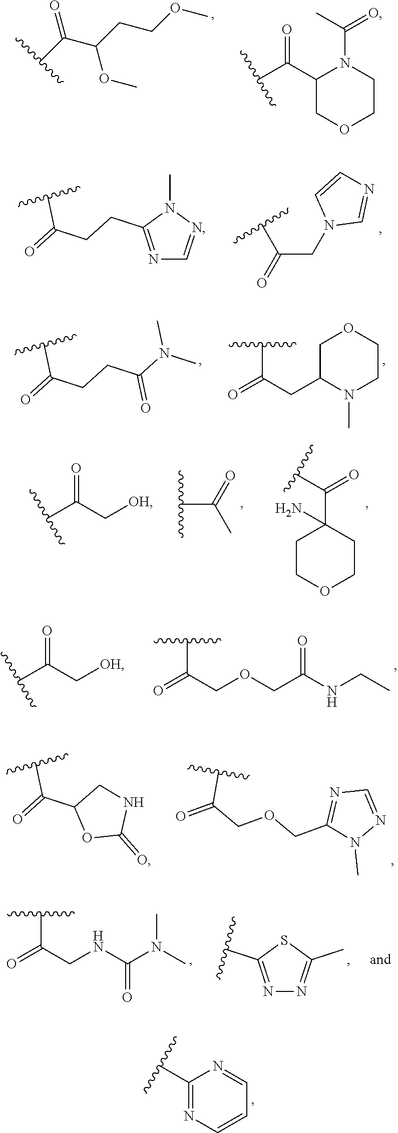
or a pharmaceutically acceptable salt thereof.

33. The compound of claim 1, wherein R^{m} is —C(O)—CH₂—O—CH₃, or a pharmaceutically acceptable salt thereof.

34. The compound of claim 1, having a formula of 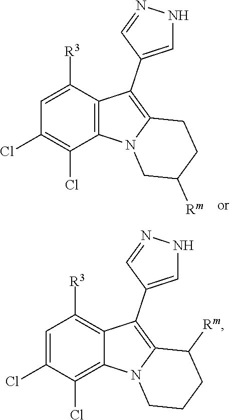
wherein:
R³ is selected from the group consisting of: H, C₁₋₄ alkoxy, 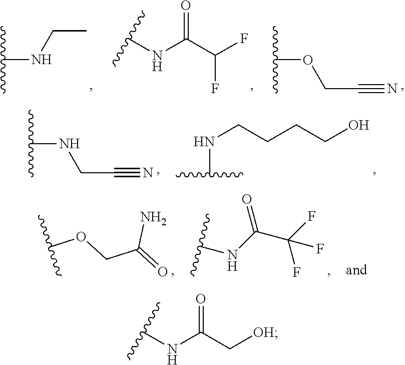
and
R^{m} is selected from the group consisting of H, —C₁₋₄ alkylene-OH, 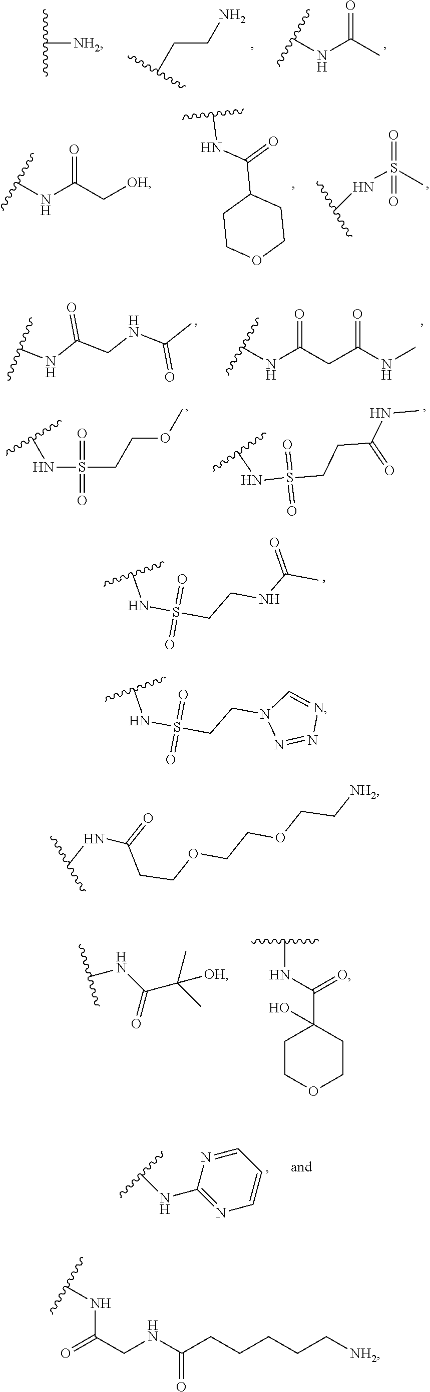
or a pharmaceutically acceptable salt thereof.

35. The compound of claim 1, having a formula of: 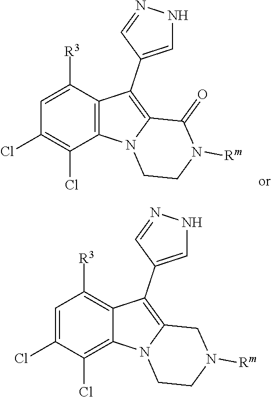
wherein:
R³ is selected from the group consisting of: 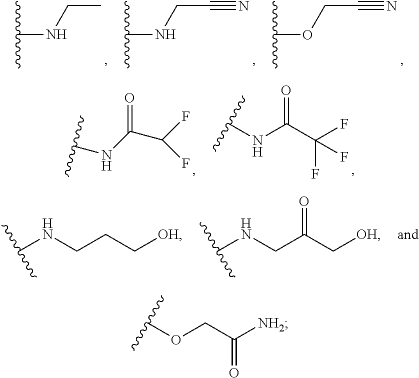
and
R^{m} is selected from the group consisting of H, —C₁₋₄ alkylene-OH, —C₁₋₄ alkoxy, 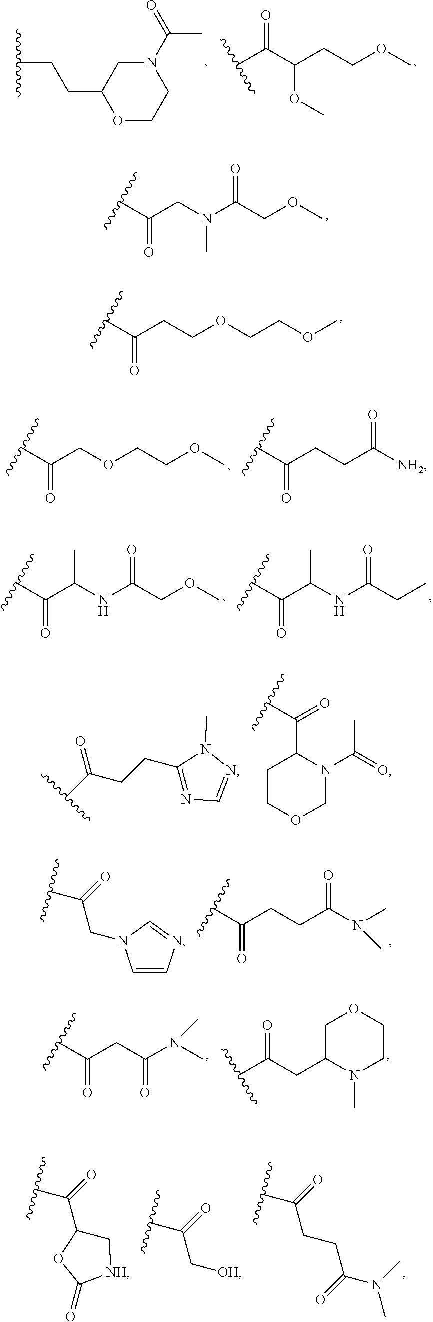 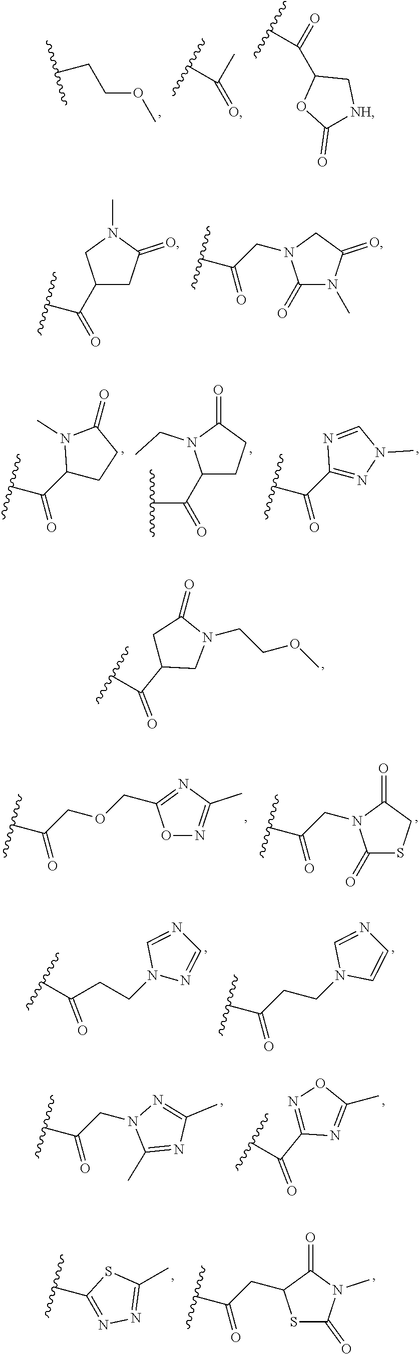 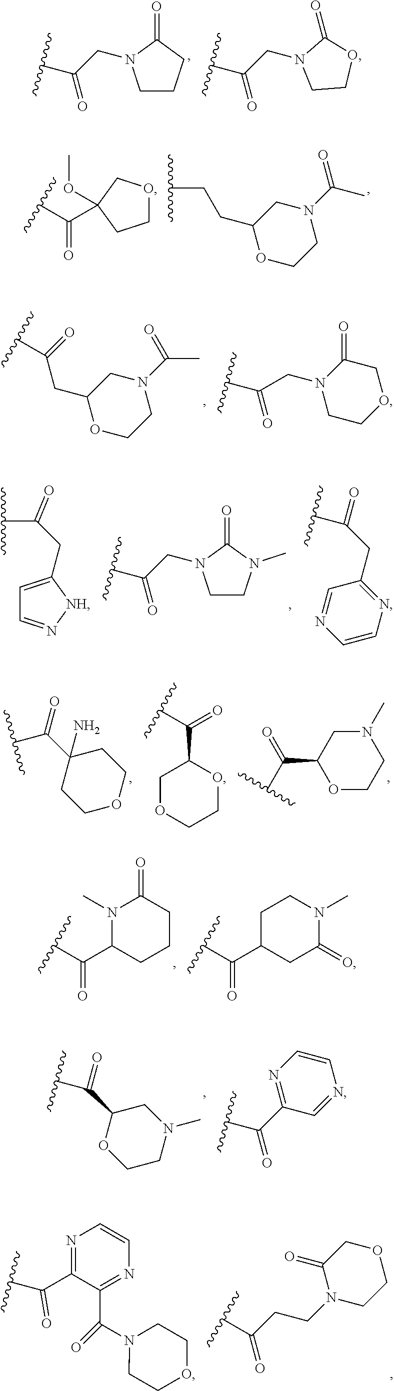 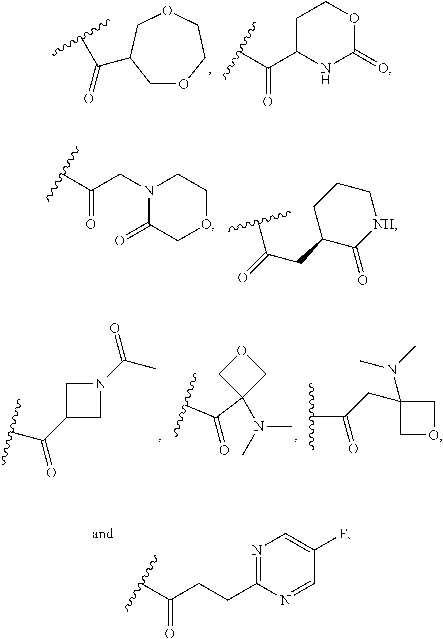
or a pharmaceutically acceptable salt thereof.

36-37. (canceled)

38. The compound of claim 1, wherein X is chloro, and R₄ is chloro, or a pharmaceutically acceptable salt thereof.

39. The compound according to claim 1, wherein the compound is selected from Tables 3 and 6, or a pharmaceutically acceptable salt thereof.

40. A pharmaceutical composition, comprising a compound according to claim 1, or a pharmaceutically acceptable salt thereof, with one or more pharmaceutically acceptable carriers, diluents, or excipients.

41. A method of treating an immune-mediated disease in a patient, comprising administering to a patient in need of such treatment an effective amount of a compound or salt according to claim 1.

42. The method of claim 41, wherein the immune-mediated disease is systemic lupus erythematosus, lupus nephritis, dermatomyositis, or Aicardi-Goutières syndrome.

43-45. (canceled)

46. A method of treating a disease associated with cGAS activation in a patient, comprising administering to a patient in need of such treatment an effective amount of a compound or salt according to claim 1.

47-79. (canceled)
